(19) United States
(12) Patent Application Publication
(10) Pub. No.: US 2025/0154169 A1
(43) Pub. Date: May 15, 2025

(54) COMPOUNDS AND METHODS FOR MODULATING SPLICING

(71) Applicant: REMIX THERAPEUTICS INC., Watertown, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Peter Smith, Arlington, MA (US); Sudeep Prajapati, Somerville, MA (US); Allen T. Hopper, Lexington, MA (US); Stepan Vyskocil, Benesov (CZ); Gulseher Sarah Sarah Sirin, Watertown, MA (US)

(21) Appl. No.: 18/688,087

(22) PCT Filed: Aug. 30, 2022
(86) PCT No.: PCT/US2022/075684

§ 371 Date: Feb. 29, 2024

Related U.S. Application Data
(60) Provisional application No. 63/393,205, filed on Jul. 28, 2022.
(60) Provisional application No. 63/393,206, filed on Jul. 28, 2022.
(60) Provisional application No. 63/283,132, filed on Nov. 24, 2021.
(60) Provisional application No. 63/282,906, filed on Nov. 24, 2021.
(60) Provisional application No. 63/238,691, filed on Aug. 30, 2021.
(60) Provisional application No. 63/238,694, filed on Aug. 30, 2021.

(51) Int. Cl. C07D 519/00; A61K 31/437; A61K 31/438; A61K 31/454; A61K 31/4545; A61K 31/496; A61K 31/498; A61K 31/4985; A61K 31/5025; A61K 31/506; A61K 31/519; A61K 31/55; C07D 403/12; C07D 403/14; C07D 413/14; C07D 417/14; C07D 471/04; C07D 487/04; C07D 498/04

(52) U.S. Cl. CPC C07D 519/00; A61K 31/437; A61K 31/438; A61K 31/454; A61K 31/4545; A61K 31/496; A61K 31/498; A61K 31/4985; A61K 31/5025; A61K 31/506; A61K 31/519; A61K 31/55; C07D 403/12; C07D 403/14; C07D 413/14; C07D 417/14; C07D 471/04; C07D 487/04; C07D 498/04

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application claims priority to U.S. Application No. 63/238,691, filed on Aug. 30, 2021; U.S. Application No. 63/238,694, filed on Aug. 30, 2021; U.S. Application No. 63/282,906, filed on Nov. 24, 2021; U.S. Application No. 63/283,132, filed on Nov. 24, 2021; U.S. Application No. 63/393,205, filed on Jul. 28, 2022; and U.S. Application No. 63/393,206, filed on Jul. 28, 2022. The disclosure of each of the foregoing applications is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)) and pharmaceutically acceptable salts, solvates, hydrates, tautomers, or stereoisomers thereof. The present disclosure additionally provides methods of using the compounds of the invention (e.g., compounds of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m), and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m), and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m), and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m), and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In another aspect, the present disclosure features a compound of Formula (I):

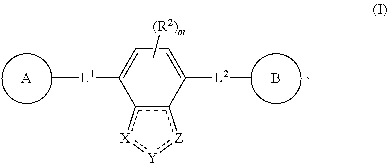

or a pharmaceutically acceptable salt, solvate, hydrate, tautomer, or stereoisomer thereof, wherein A, B, X, Y, Z, L¹, L², R², m, and subvariables thereof are as described herein.

In another aspect, the present disclosure features a compound of Formula (II):

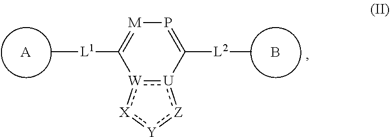

or a pharmaceutically acceptable salt, solvate, hydrate, tautomer, or stereoisomer thereof, wherein A, B, M, P, W, U, X, Y, Z, L¹, L², and subvariables thereof are as described herein

In another aspect, the present invention provides pharmaceutical compositions comprising a compound of Formula (I) or (II) (e.g., a compound of Formulas (I-a), (I-b), (I-c), (I-d), (I-e), (I-f), (I-g), (I-h), or (I-i)), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)) or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)) or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formula (I) or (II) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formula (I) or (II) alters a target nucleic acid (e.g., DNA or RNA, e.g., a precursor RNA, e.g., a pre-mRNA), target protein, or combination thereof. In some embodiments, the compound of Formula (I) or (II) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I) or (II), e.g., in a healthy or diseased cell or tissue). In some embodiments, the presence of a compound of Formula (I) or (II) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I) or (II), e.g., in a healthy or diseased cell or tissue).

In another aspect, the present disclosure provides methods for preventing and/or treating a disease, disorder, or condition in a subject by administering a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)) or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)) or a pharmaceutically acceptable salt, solvate, hydrate, tautomer, or stereoisomer thereof in a biological sample or subject. In another aspect, the present disclosure provides methods of altering the isoform of a target protein with a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m))) or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formula (I) or (II) to a biological sample, a cell, or a subject comprises inhibition of cell growth or induction of cell death.

In another aspect, the present disclosure provides compositions for use in preventing and/or treating a disease, disorder, or condition in a subject by administering a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)) or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)) or a pharmaceutically acceptable salt, solvate, hydrate, tautomer, or stereoisomer thereof in a biological sample or subject. In another aspect, the present disclosure provides compositions for use in altering the isoform of a target protein with a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m))) or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formula (I) or (II) to a biological sample, a cell, or a subject comprises inhibition of cell growth or induction of cell death.

In another aspect, the present disclosure features kits comprising a container with a compound of Formula (I) or (II) (e.g., a compound of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m)), or a pharmaceutically acceptable salt, solvate, hydrate, tautomer, stereoisomer thereof, or a pharmaceutical composition thereof. In certain embodiments, the kits described herein further include instructions for administering the compound of Formula (I) or (II) or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2021/174165. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and and WO 2021/174165, each of which is incorporated herein by reference in its entirety. The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention. As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—CI₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 16-membered non-aromatic ring system having ring carbon atoms and 1 to 8 ring heteroatoms, wherein each heteroatom is independently selected from nitrogen, oxygen, sulfur, boron, phosphorus, and silicon (“3-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein “oxo” refers to a carbonyl, i.e., —C(O)—.

The symbol “ [symbol] ” as used herein in relation to a compound of Formula (I) or (II) refers to an attachment point to another moiety or functional group within the compound. Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) or (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomer” refers to compounds that are interchangeable forms of a particular compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure. The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formula (I) or (II) refers to an amount sufficient to elicit the desired biological response, i.e., treating the condition. As will be appreciated by those of ordinary skill in this art, the effective amount of a compound of Formula (I) or (II) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I) or (II) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formula (I) or (II)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formula (I) or (II)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

The present disclosure features a compound of Formula (I):

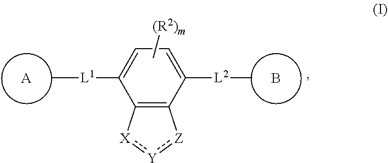

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; X, Y, and Z are each independently C(R^{3a}), C(R^{3a})(R^{3b}), N, N(R^{3c}), or O, wherein at least one of X, Y, and Z is N, N(R^{3c}), or O, and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In another aspect, the present disclosure features a compound of Formula (II):

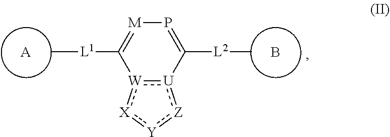

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; M and P are each independently C(R²) or N; U and W are each independently C or N; X, Y, and Z are each independently C(R^{3a}), N, N(R^{3c}), O, or S, wherein at least one of X, Y, and Z is N or N(R^{3c}), and the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁—C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R¹¹; each R⁴ is independently hydrogen, C₁-C₆-alkyl, C₁-C₆-heteroalkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2 As generally described herein for Formulas (I) and (II), A and B, are each independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, B is a nitrogen-containing bicyclic heteroaryl (e.g., a 9-membered nitrogen-containing bicyclic heteroaryl), that is optionally substituted with one or more R¹. In some embodiments, B is a 9-membered bicyclic heteroaryl comprising 1 nitrogen atom. In some embodiments, B is a 9-membered bicyclic heteroaryl comprising 2 nitrogen atoms. In some embodiments, B is a 9-membered bicyclic heteroaryl comprising 3 nitrogen atoms. In some embodiments, B is a 9-membered bicyclic heteroaryl comprising 4 nitrogen atoms. The one or more nitrogen atom of the 9-membered bicyclic heteroaryl may be at any position of the ring. In some embodiments, B is a 9-membered bicyclic heteroaryl substituted with one or more R¹.

In some embodiments, each of A and B are independently selected from:

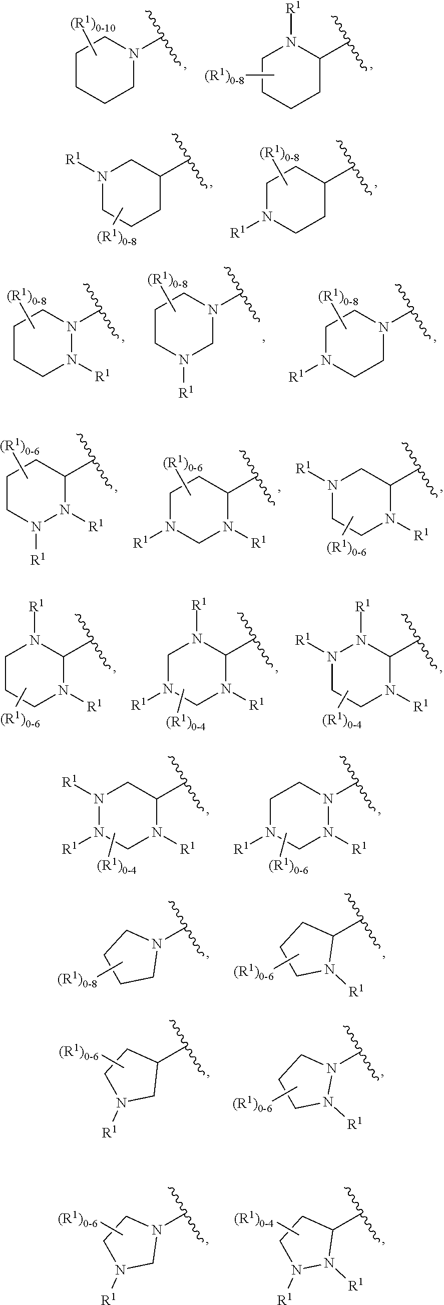

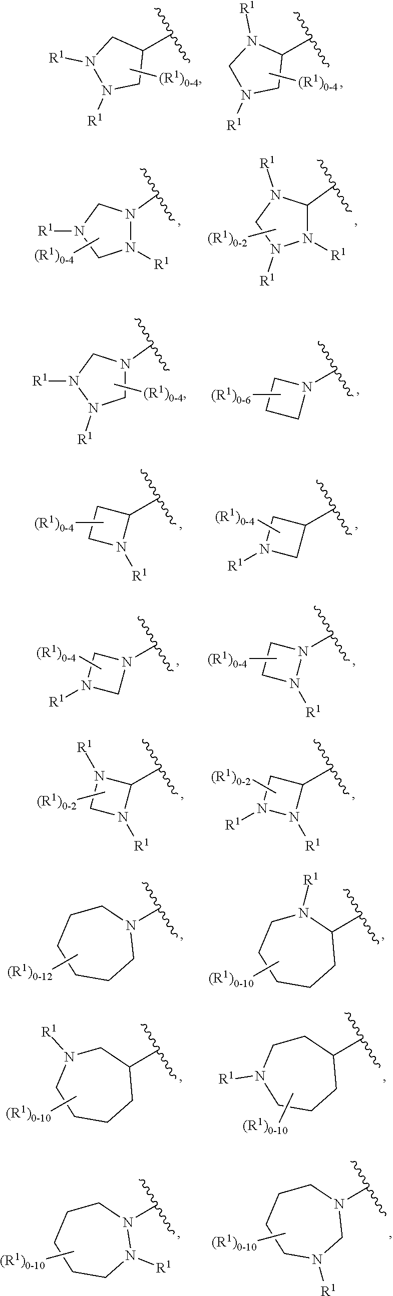

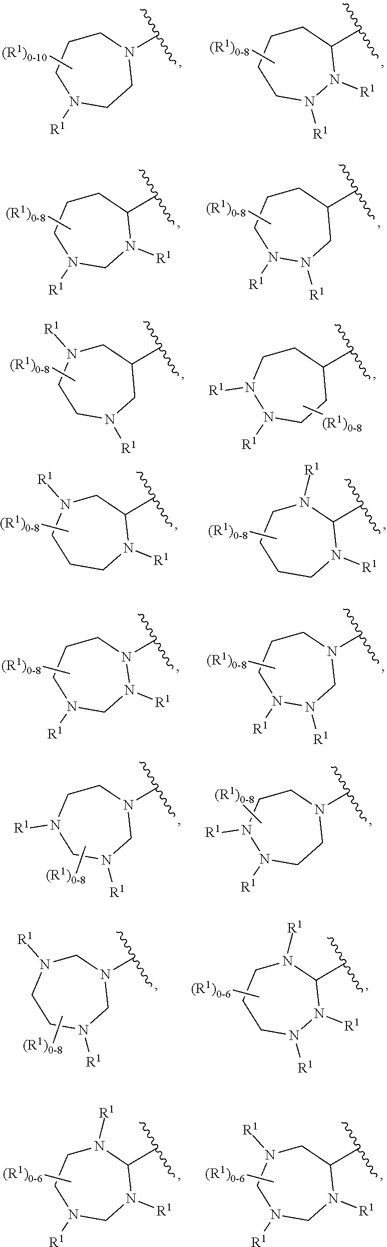

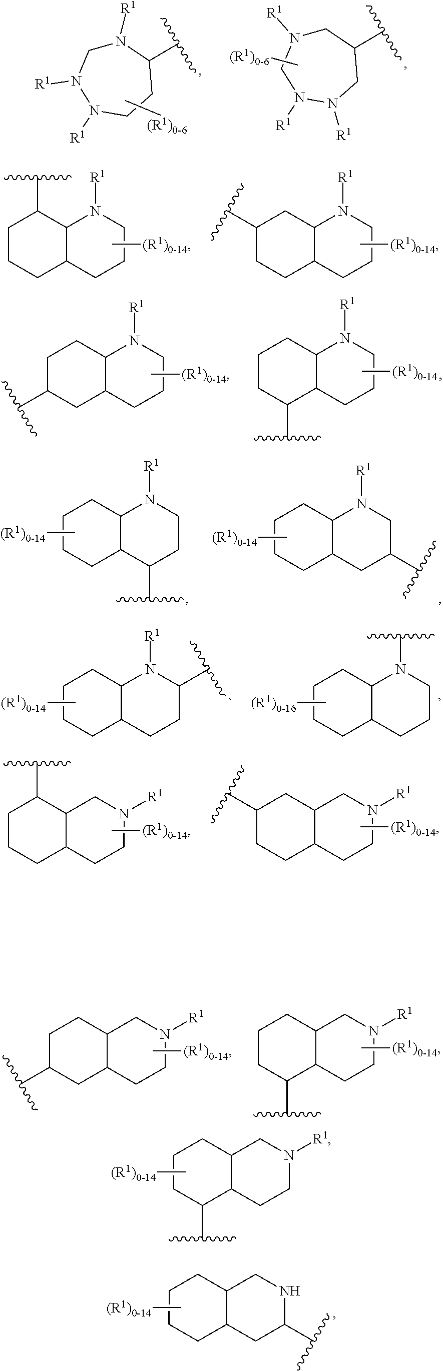

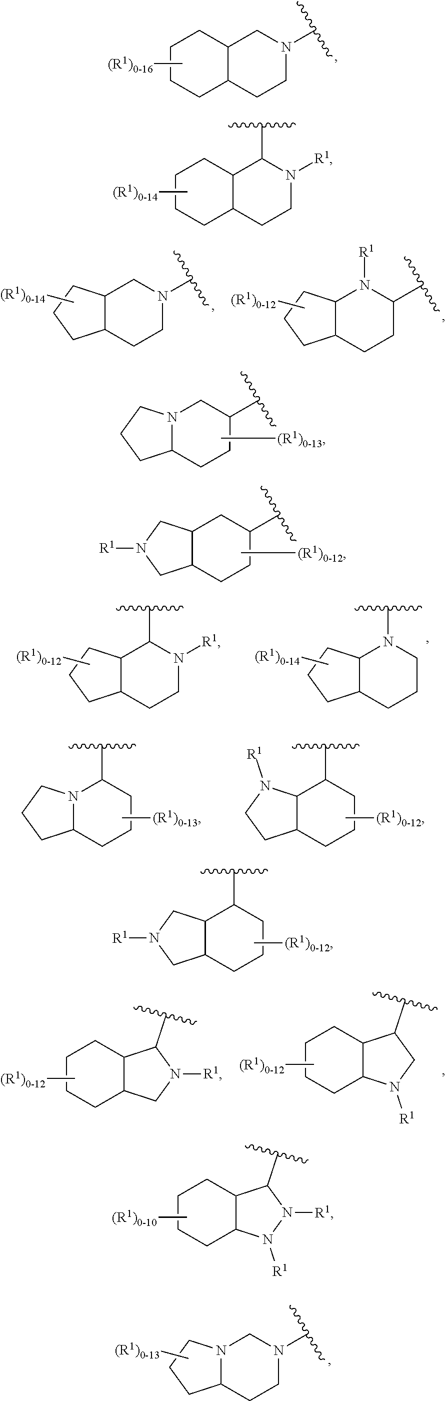

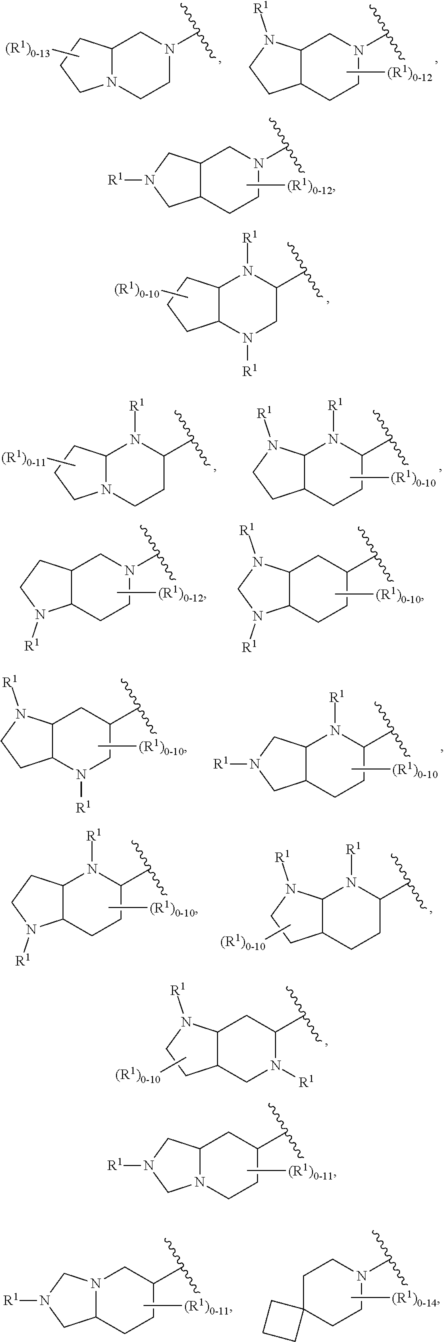

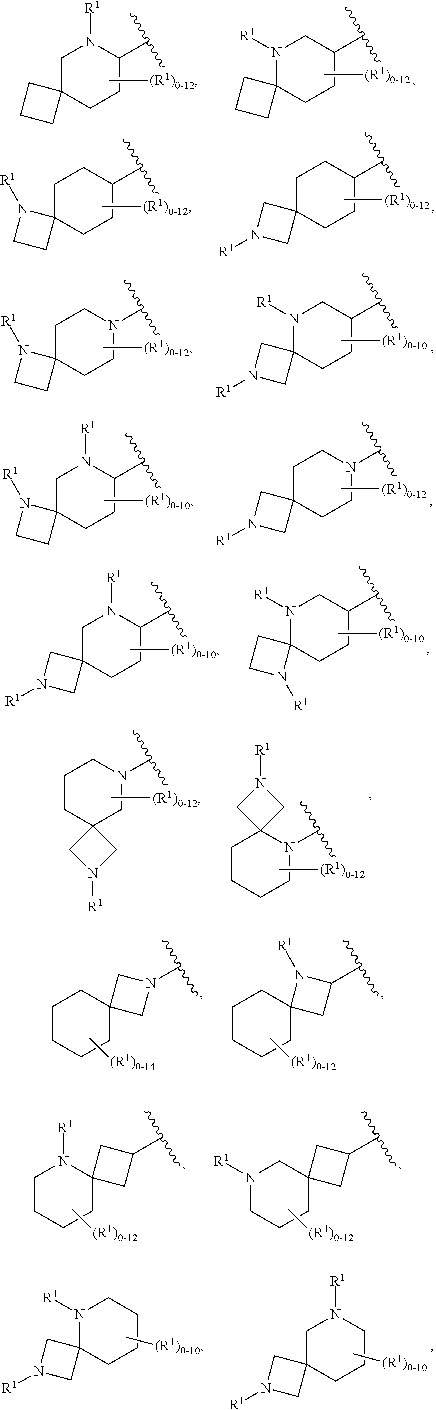

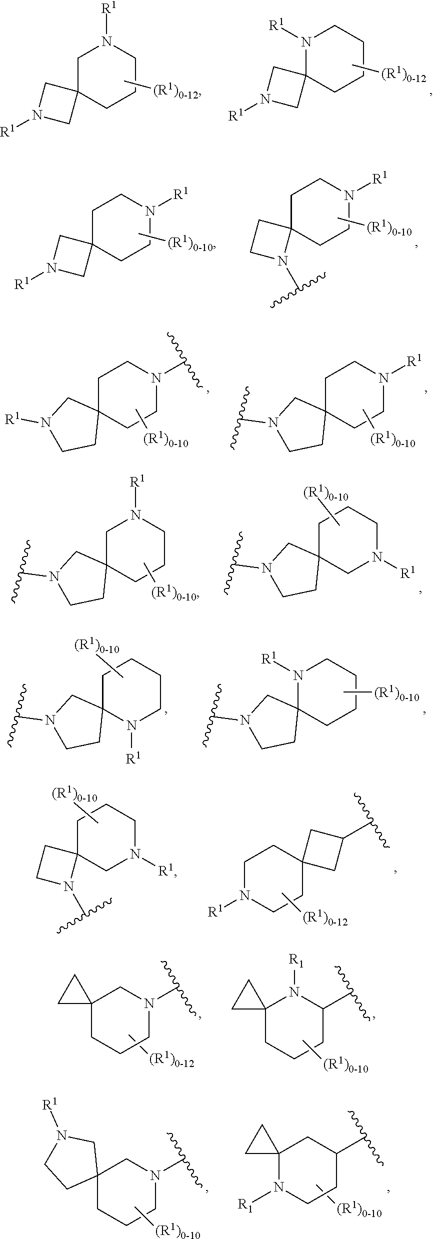

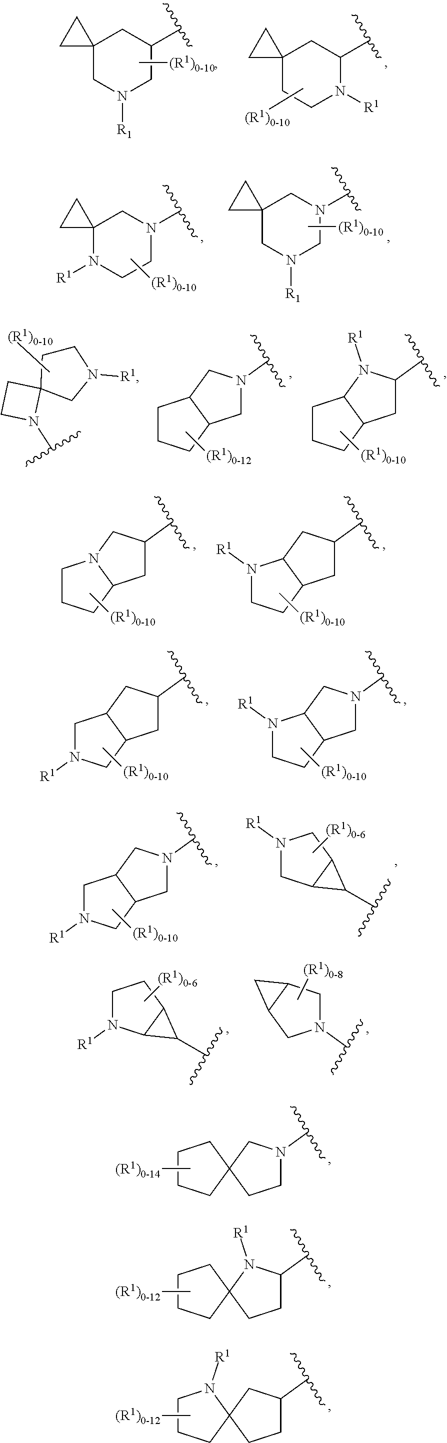

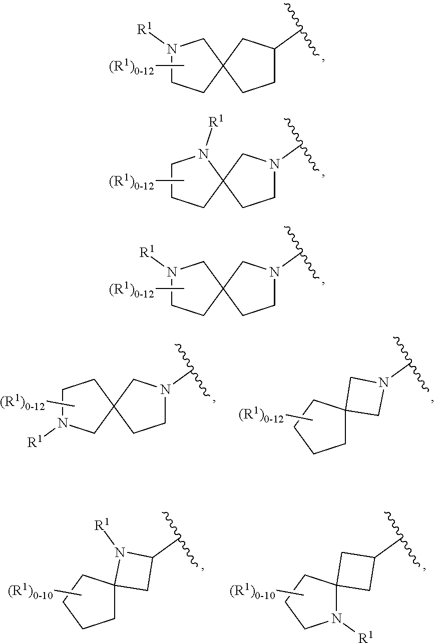

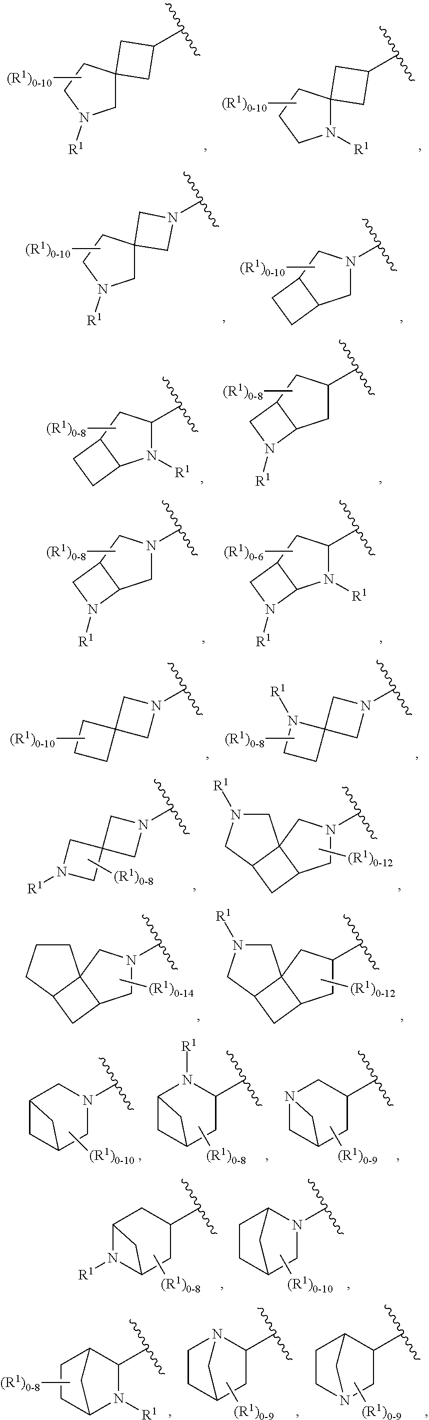

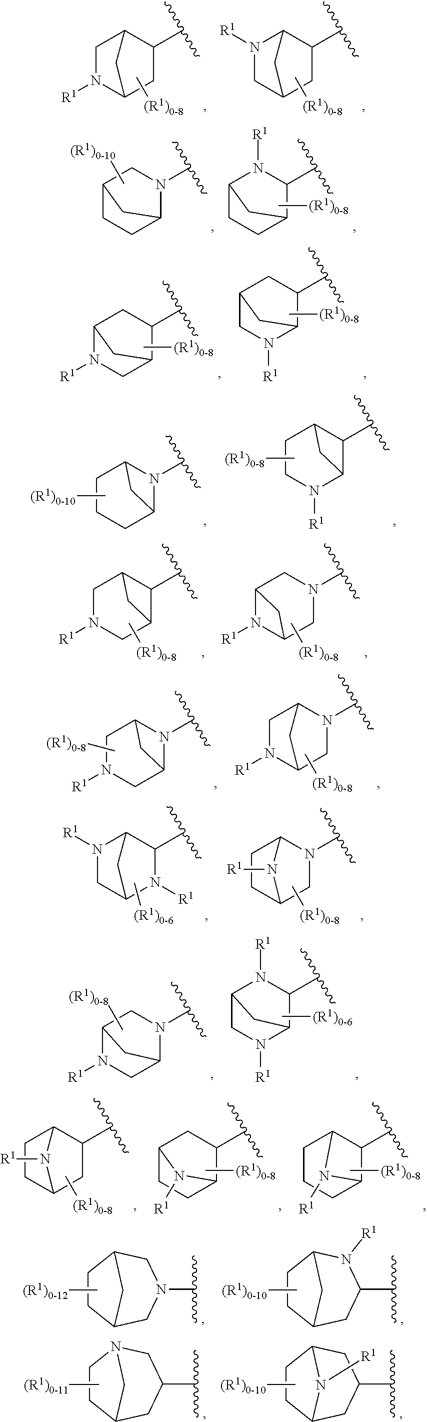

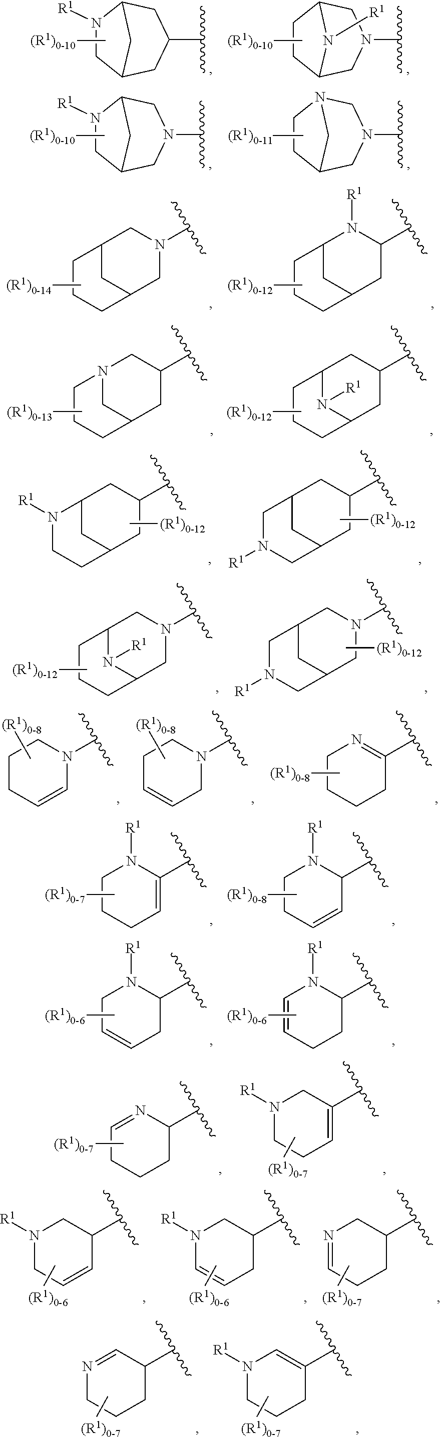

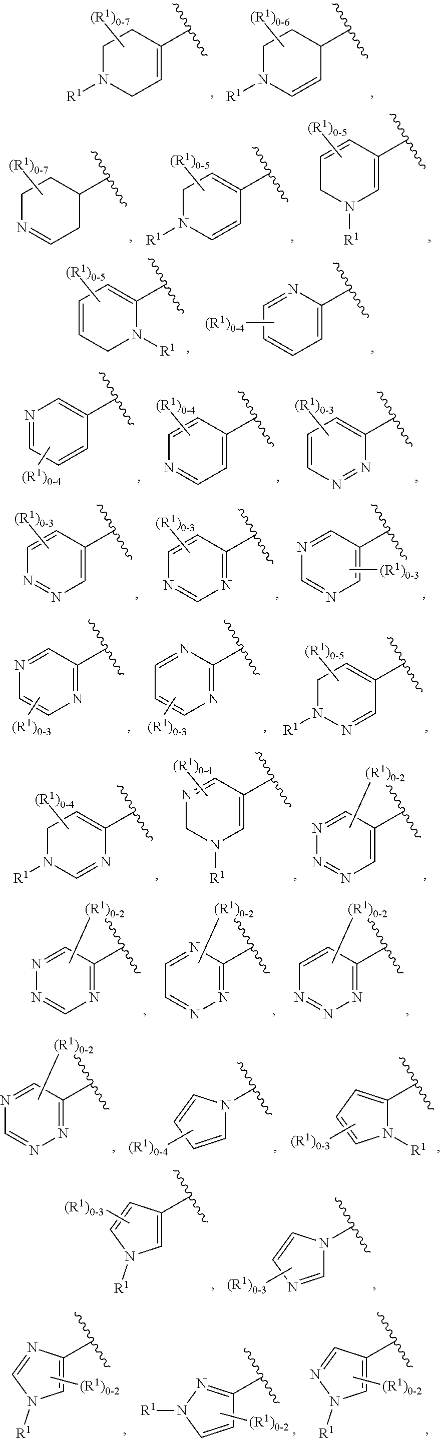

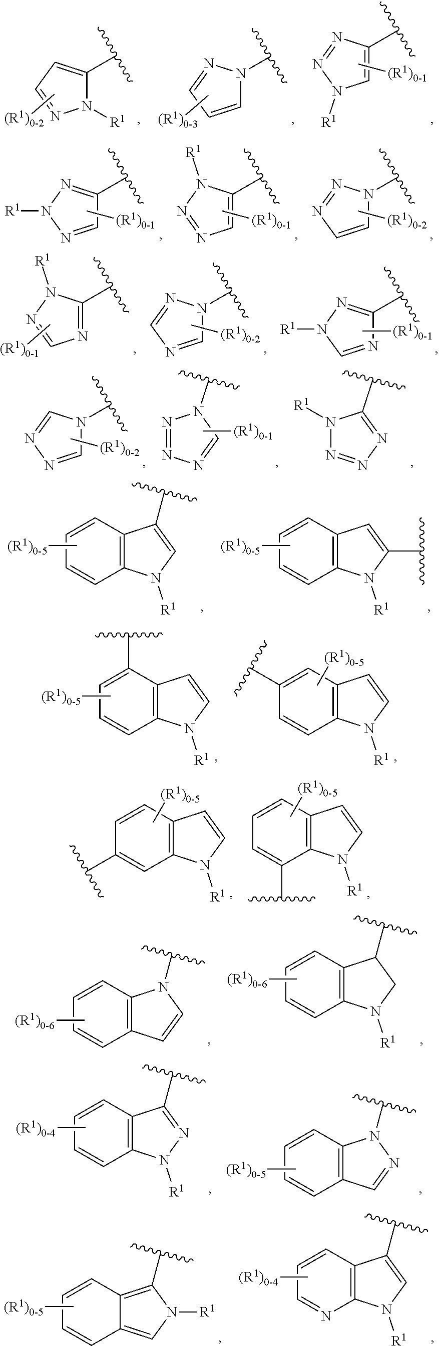

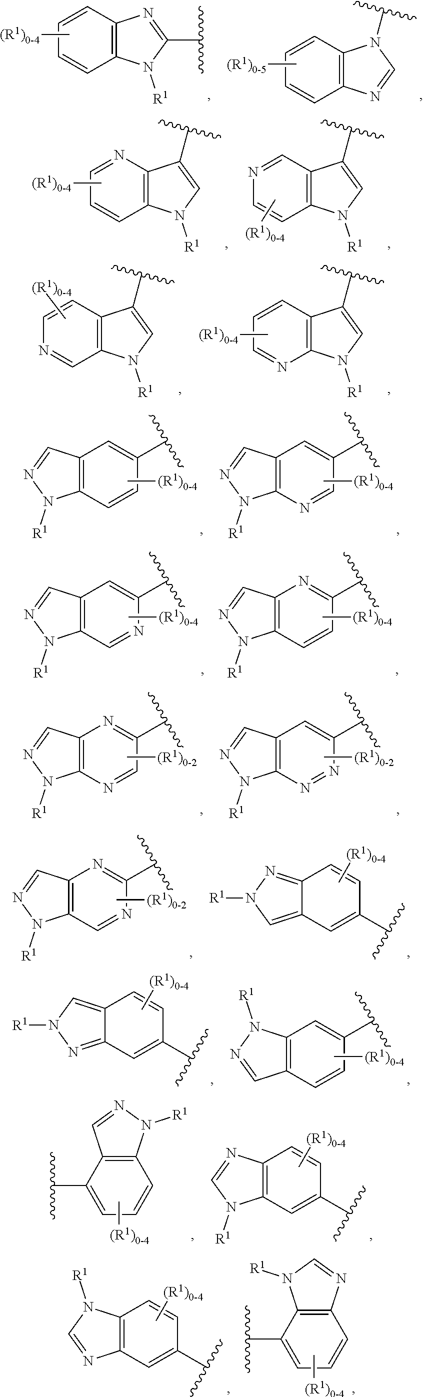

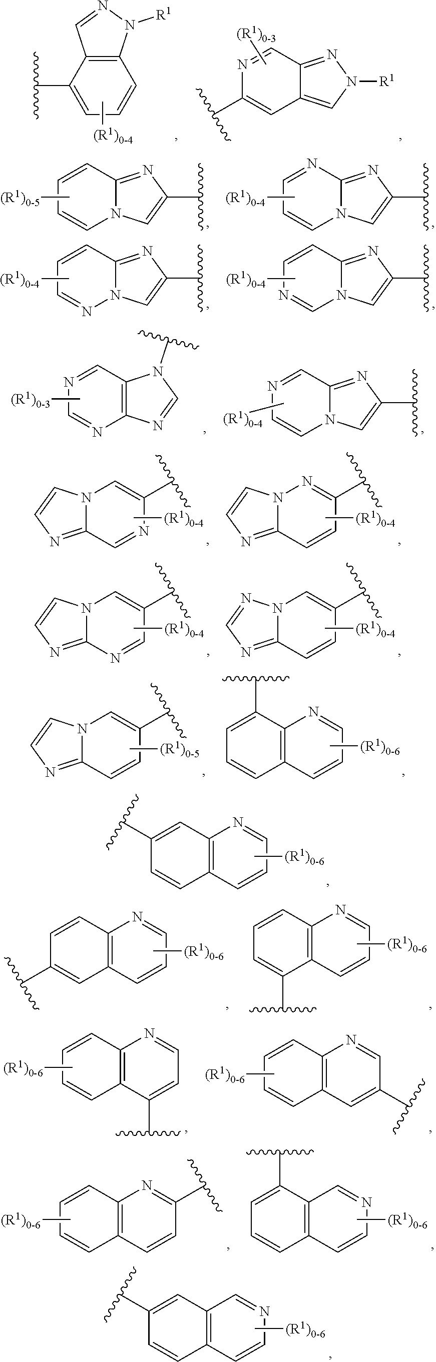

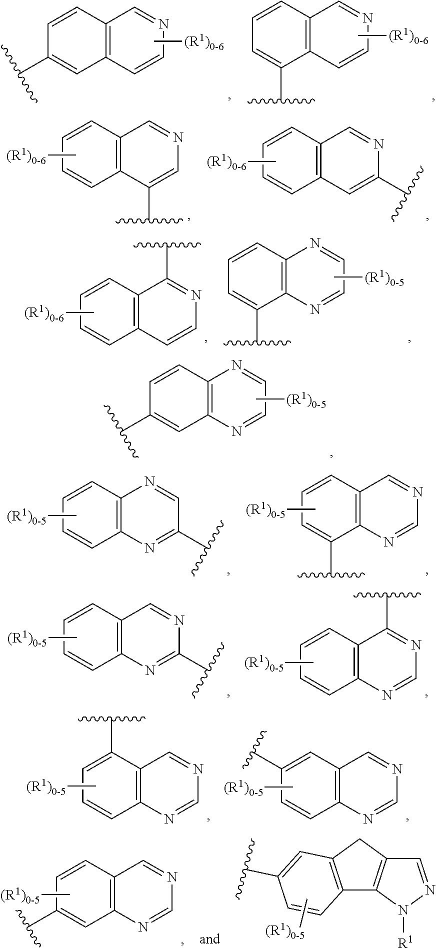

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

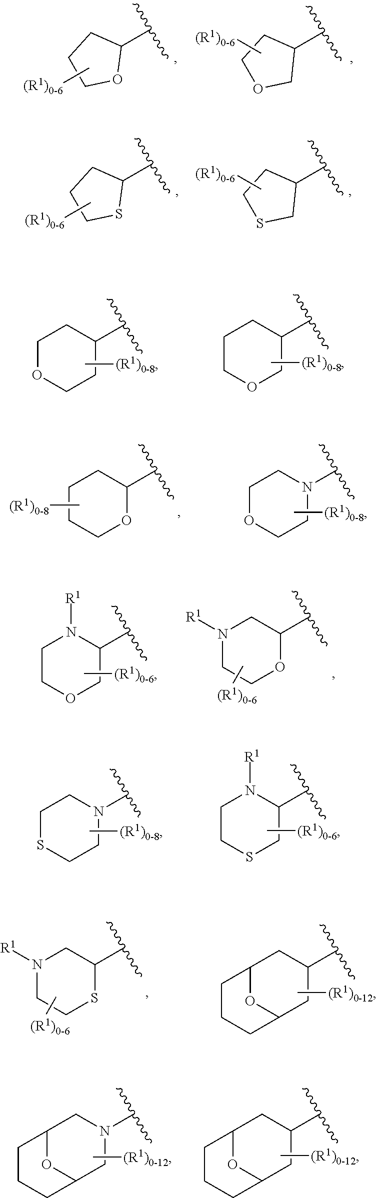

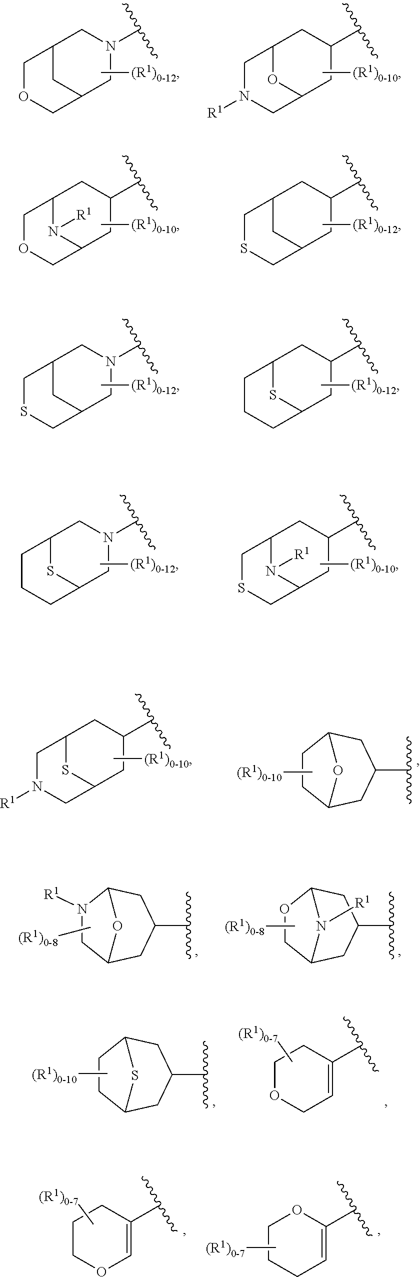

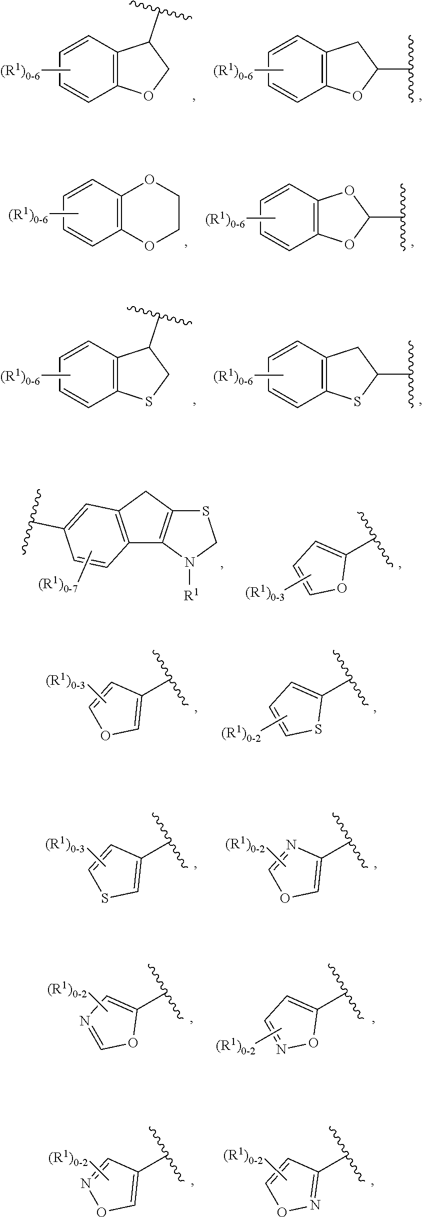

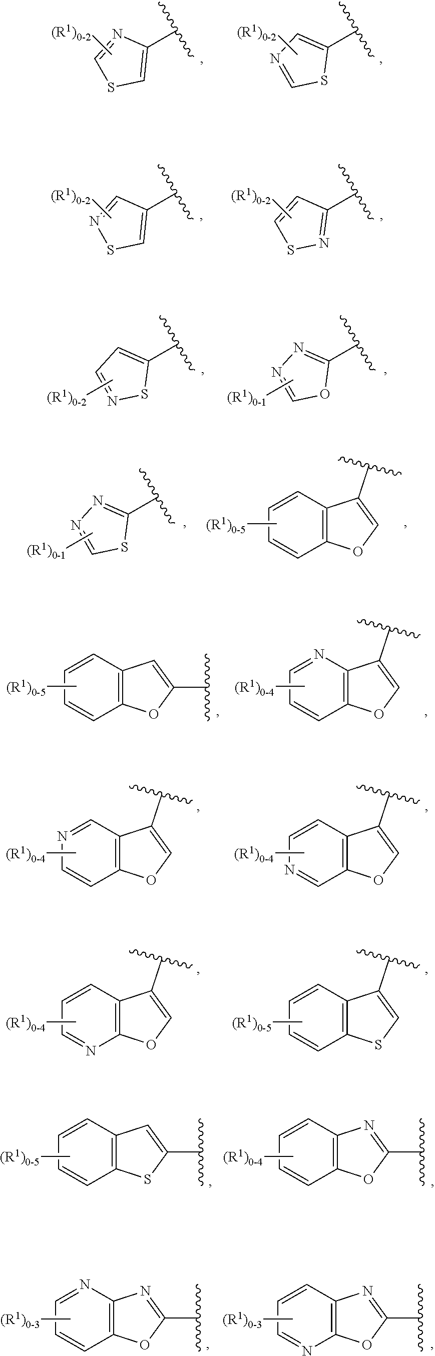

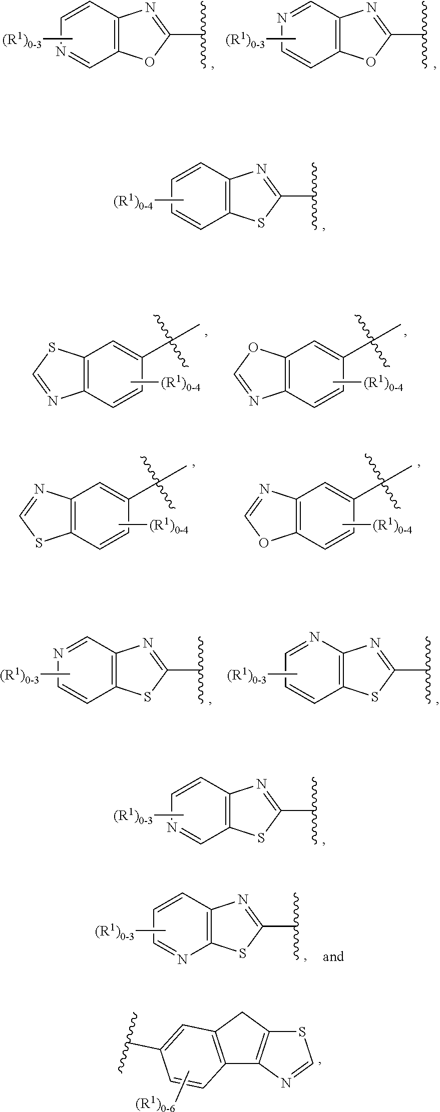

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, one of A and B is independently selected from

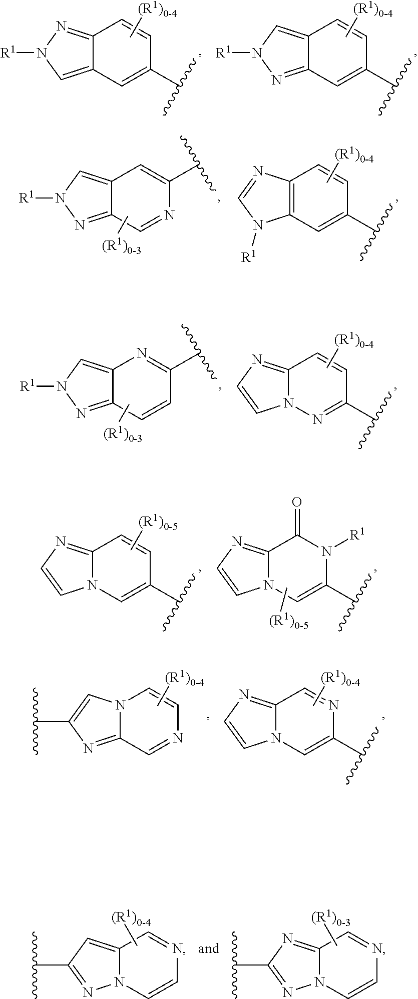

wherein R¹ is as described herein. In some embodiments, one of A and B is independently selected from

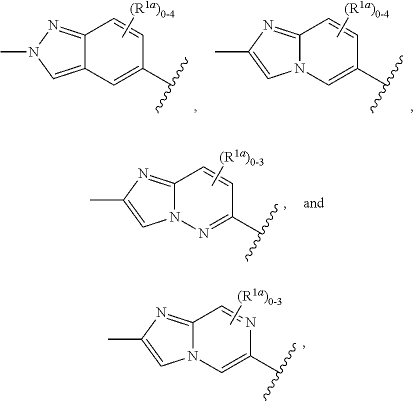

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷. In some embodiments, one of A and B is independently

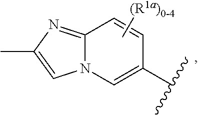

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

In some embodiments, one of A and B is independently selected from N

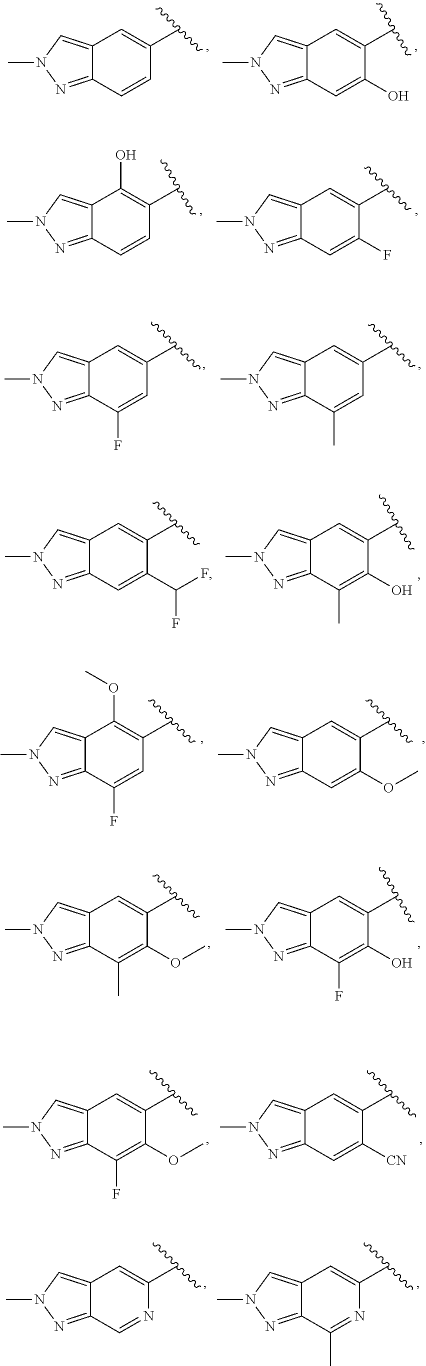

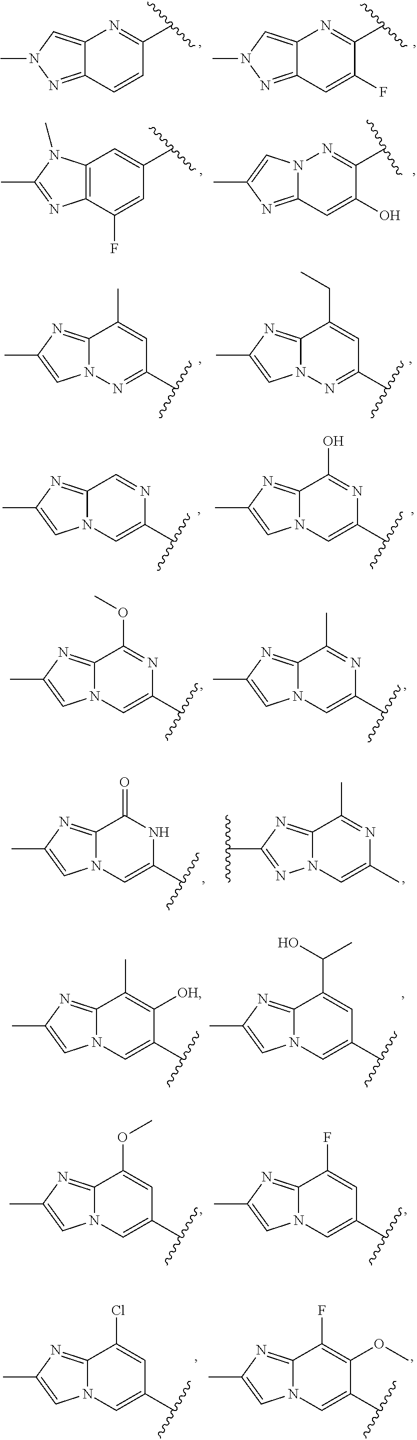

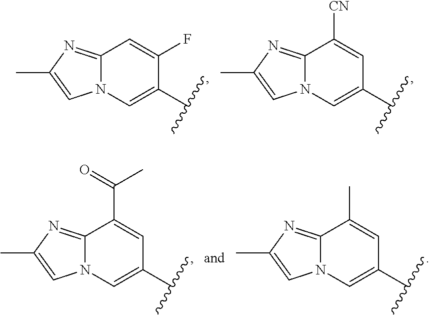 [SMILES: *c1cn2cc(C)nc2cc1F ; *c1cc(C#N)c2nc(C)cn2c1 ; *c1cc(C(C)=O)c2nc(C)cn2c1 ; *c1cc(C)c2nc(C)cn2c1]

In some embodiments, one of A and B is independently selected from

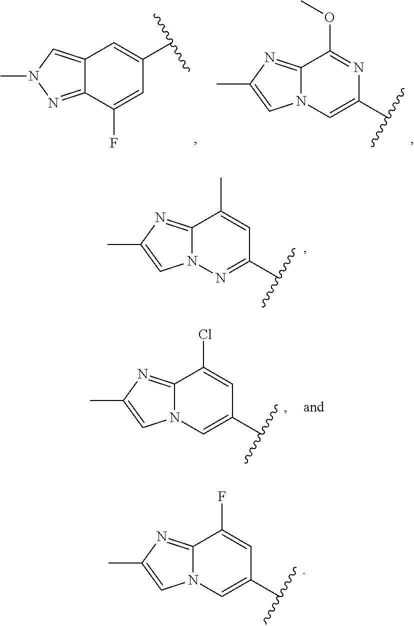 [SMILES: *c1cc(F)c2nn(C)cc2c1 ; *c1cn2cc(C)nc2c(OC)n1 ; *c1cc(C)c2nc(C)cn2n1 ; *c1cc(Cl)c2nc(C)cn2c1 ; *c1cc(F)c2nc(C)cn2c1]

In some embodiments, one of A and B is independently

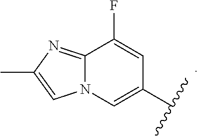 [SMILES: *c1cc(F)c2nc(C)cn2c1]

In some embodiments, one of A and B is independently a monocyclic heterocyclyl or bicyclic heterocyclyl, each of which is optionally substituted with one or more R¹. In some embodiments, one of A and B is independently a nitrogen-containing heterocyclyl optionally substituted with one or more R¹. In some embodiments, one of A and B is independently a 4-8 membered heterocyclyl optionally substituted with one or more R¹. In some embodiments, one of A and B is independently selected from

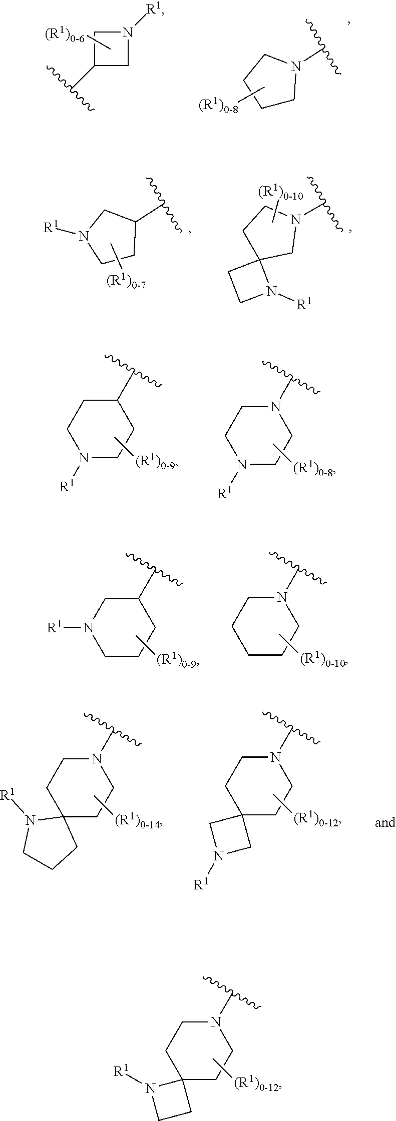

wherein R¹ is as described herein. In some embodiments, one of A and B is independently selected from

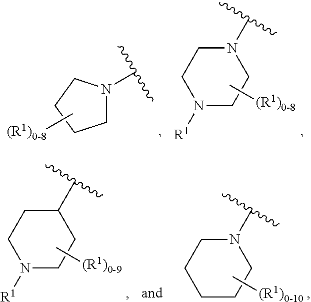

wherein R¹ is as described herein. In some embodiments, one of A and B is

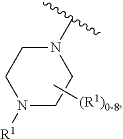

wherein R¹ is as described herein. In some embodiments, A is selected from

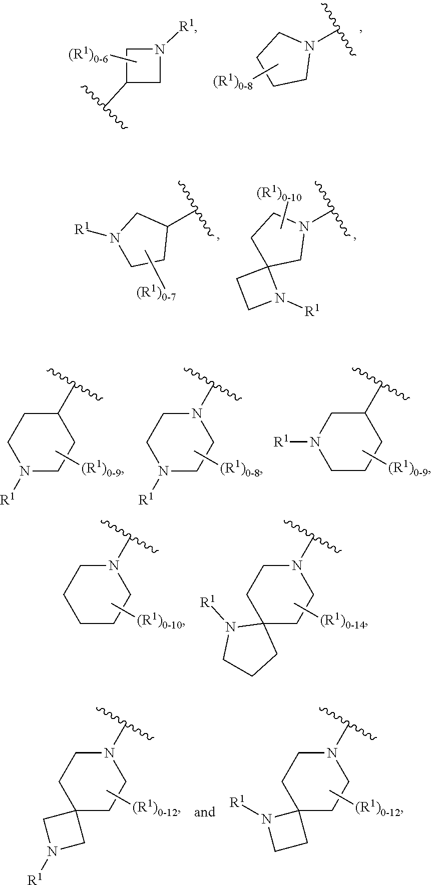

wherein R¹ is as described herein. In some embodiments, B is selected from

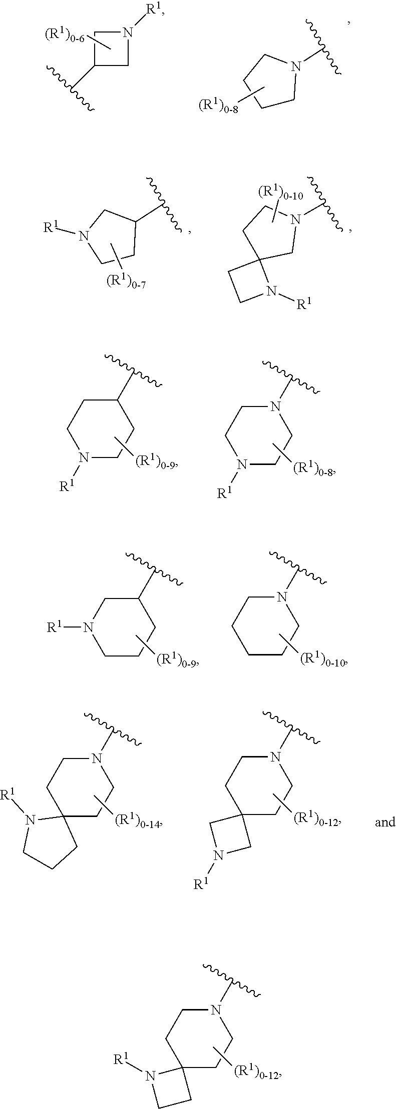

wherein R^{t} is as described herein. In some embodiments, B is selected from

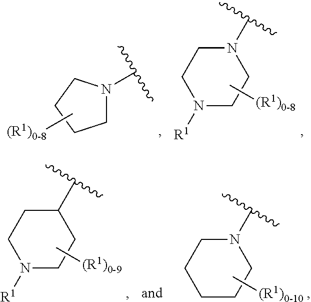

wherein R^{t} is as described herein. In some embodiments, A is selected

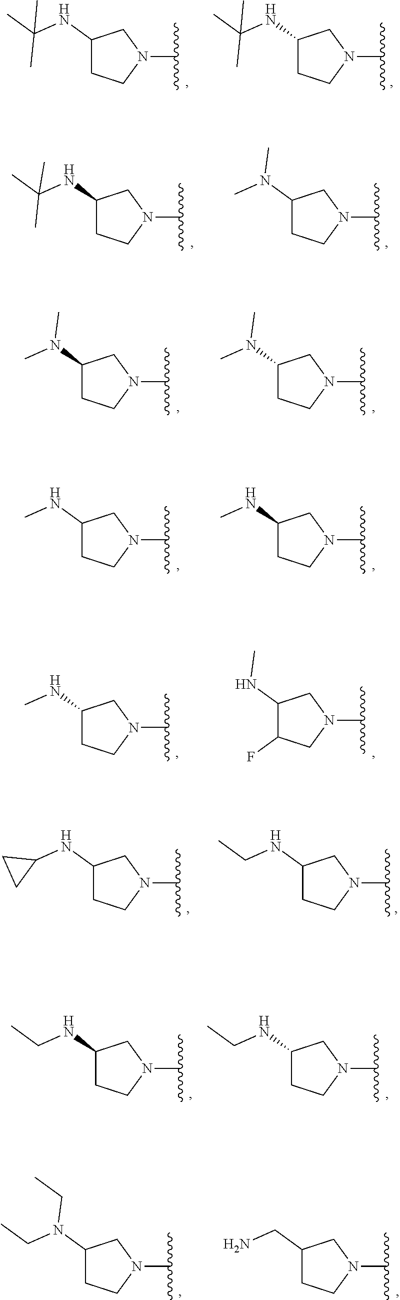

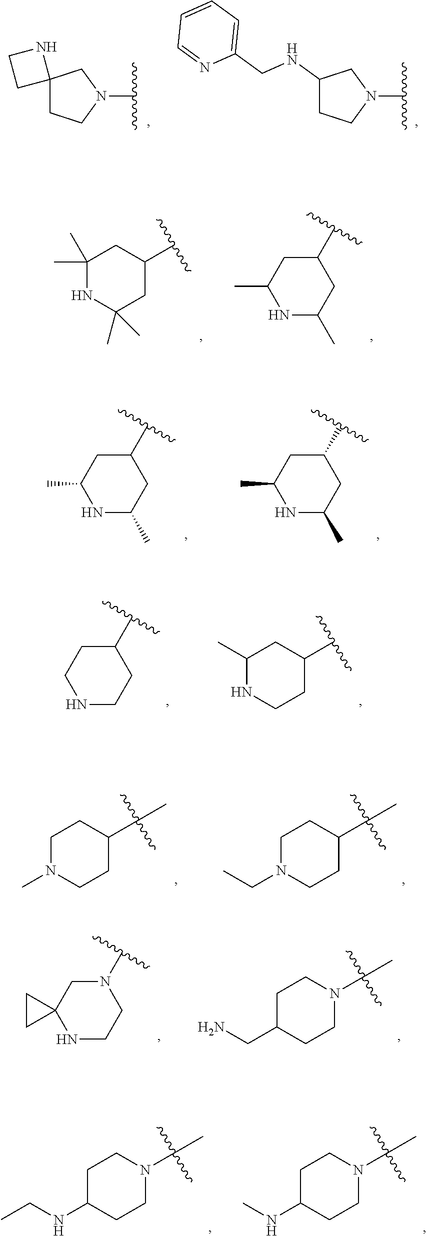

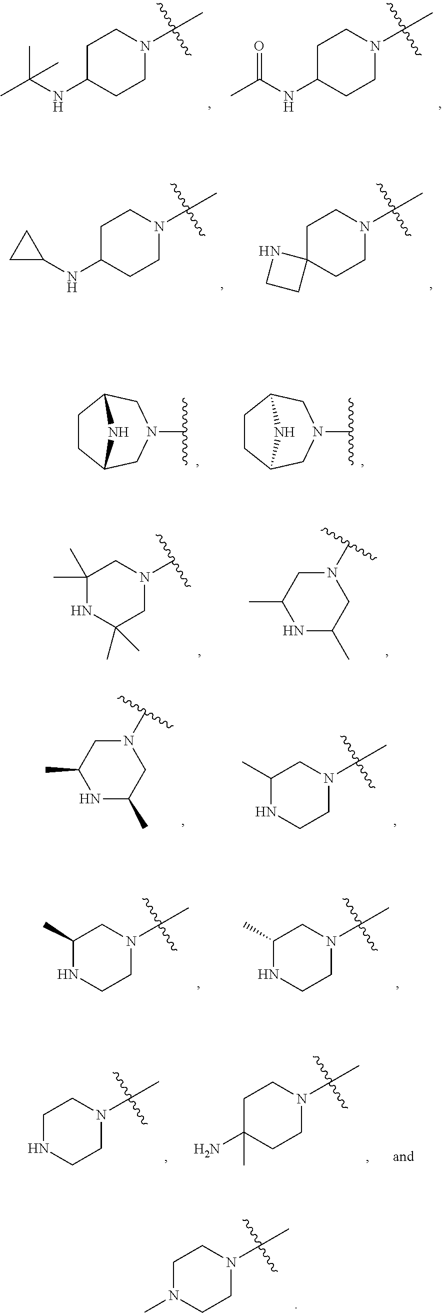

In some embodiments, A is selected one of A and B is independently selected from,

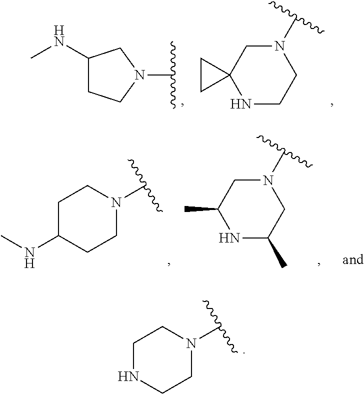

In some embodiments, one of A and Bis

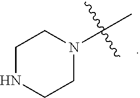 [SMILES: *N1CCNCC1]

As generally described herein, for Formulas (I) and (II), L¹ and L² each independently may be absent or refer to a C₁-C₆-alkylene, C₁-C₆-heteroalkylene, —O—, —C(O)—, —N(R⁴)—, —N(R⁴)C(O)—, or —C(O)N(R⁴)— group, wherein each alkylene and heteroalkylene is optionally substituted with one or more R⁵.

In some embodiments, L¹ is absent. In some embodiments, L¹ is C₁-C₆-alkylene (e.g., C₁-alkylene, C₂-alkylene, C₃-alkylene, C₄-alkylene, C₅-alkylene, or C₆-alkylene). In some embodiments, L¹ is unsubstituted C₁-C₆ alkylene. In some embodiments, L¹ is substituted C₁-C₆-alkylene, e.g., C₁-C₆ alkylene substituted with one or more R⁵. In some embodiments, L¹ is C₁-alkylene substituted with one R⁵. In some embodiments, L¹ is —CH₂— (or methylene). In some embodiments, L¹ is —C(O)— (or carbonyl).

In some embodiments, L¹ is absent, C₁-C₆-alkylene, C₁-C₆-heteroalkylene, —N(R⁴)C(O)—, or —C(O)N(R⁴)—, wherein each alkylene and heteroalkylene is optionally substituted with one or more R⁵.

In some embodiments, L¹ is C₁-C₆ heteroalkylene (e.g., C₁-heteroalkylene, C₂-heteroalkylene, C₃-heteroalkylene, C₄-heteroalkylene, C₅-heteroalkylene, or C₆-heteroalkylene). In some embodiments, L¹ is unsubstituted C₁-C₆ heteroalkylene. In some embodiments, L¹ is substituted heteroalkylene, e.g., C₁-C₆ heteroalkylene substituted with one or more R⁵. In some embodiments, the heteroalkylene comprises 1 or more heteroatoms. In some embodiments, the heteroalkylene comprises one or more of oxygen, sulfur, nitrogen, boron, silicon, or phosphorus. In some embodiments, L¹ is —N(R⁴)C(O)—. In some embodiments, L¹ is —C(O)N(R⁴)—. In some embodiments, L¹ is —C(O)N(H)—.

In some embodiments, L¹ is oxygen. In some embodiments, L¹ is nitrogen which is optionally substituted with R⁴. In some embodiments, L¹ is nitrogen substituted with R⁴. In some embodiments, L¹ is —N(R⁴)—, e.g., —N(CH₃)—. In some embodiments, L¹ is —NH—. In some embodiments, L¹ is —O—.

In some embodiments, L² is absent, C₁-C₆-alkylene, C₁-C₆-heteroalkylene, —N(R⁴)C(O)—, or —C(O)N(R⁴)—, wherein each alkylene and heteroalkylene is optionally substituted with one or more R⁵. In some embodiments, L² is unsubstituted C₁-C₆ heteroalkylene. In some embodiments, L² is substituted heteroalkylene, e.g., C₁-C₆ heteroalkylene substituted with one or more R⁵. In some embodiments, the heteroalkylene comprises 1 or more heteroatoms. In some embodiments, the heteroalkylene comprises one or more of oxygen, sulfur, nitrogen, boron, silicon, or phosphorus. In some embodiments, L² is —N(R⁴)C(O)—. In some embodiments, L² is —C(O)N(R⁴)—. In some embodiments, L² is —C(O)N(H)—.

In some embodiments, L² is nitrogen which is optionally substituted with R⁴. In some embodiments, L² is nitrogen substituted with R⁴. In some embodiments, L² is —N(R⁴)—, e.g., —N(CH₃)—. In some embodiments, L² is —NH—.

As generally described herein, for Formula (I), X, Y, and Z each independently refer to C(R^{3a}), C(R^{3a})(R^{3b}), N, or N(R^{3c}), or O. In some embodiments, at least one of X, Y, and Z is either N or N(R^{3c}). In some embodiments, at least one of X, Y, and Z is O. In some embodiments, at least two of X, Y, and Z is N or N(R^{3c}). In some embodiments, X is N. In some embodiments, X is N(R^{3c}). In some embodiments, X is O. In some embodiments, X is C(R^{3a}) (e.g., CH). In some embodiments, X is C(R^{3a})(R^{3b}). In some embodiments, Y is N. In some embodiments, Y is N(R^{3c}). In some embodiments, Y is C(R^{3a}) (e.g., CH). In some embodiments, Y is C(R^{3a})C(R^{3b}). In some embodiments, Z is N. In some embodiments, Z is N(R^{3c}). In some embodiments, Z is C(R^{3a}) (e.g., CH). In some embodiments, Z is C(R^{3a})C(R^{3b}) In some embodiments, two of X, Y, and Z are N, and the other of X, Y, and Z is C(R^{3a}) (e.g., CH). In some embodiments, one of X, Y, and Z is C(R^{3a}) (e.g., CH), and the others of X, Y, and Z are each independently N. In some embodiments, X and Y are each independently N, and Z is C(R^{3a}) (e.g., CH). In some embodiments, X is C(R^{3a}) (e.g., CH), and Y and Z are each independently N.

In some embodiments, X, Y, and Z are each independently N or C(R^{3a}), wherein at least one of X, Y, and Z is N and the bonds in the ring comprising X, Y, and Z may be single or double bonds as valency permits.

In some embodiments, X is C(R^{3a}), Y is C(R^{3a}), and Z is O. In some embodiments, X is C(R^{3a}), Y is C(R^{3a}), Z is O, and y is 0. In some embodiments, X is C(R^{3a}), Y is C(R^{3a}), Z is O, and the bond between X and Y is a double bond. In some embodiments, X is C(R^{3a}), Y is C(R^{3a}), Z is O, and the bond between Y and Z is a single bond.

In some embodiments,

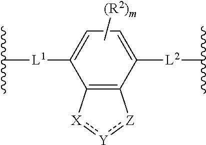

is selected from

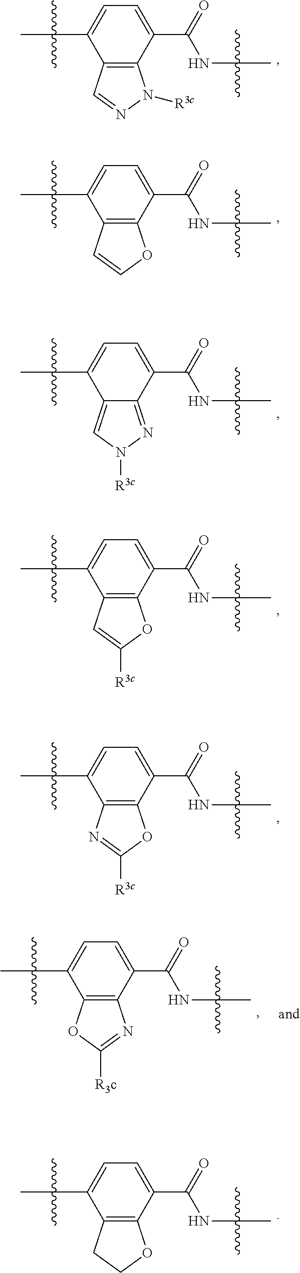

In some embodiments,

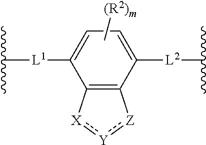

is

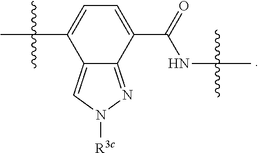

In some embodiments,

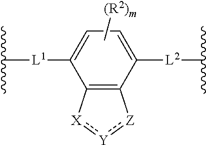

is

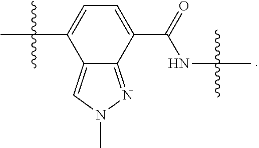 [SMILES: *NC(=O)c1ccc(*)c2cn(C)nc12]

In some embodiments,

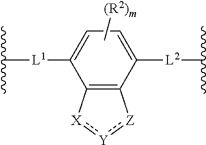

is

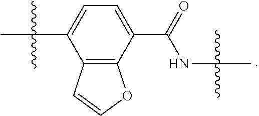 [SMILES: *NC(=O)c1ccc(*)c2ccoc12]

In some embodiments for Formulas (I) and (II), R¹ is hydrogen. In some embodiments, R¹ is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁶. In some embodiments, R¹ is C₂-C₆-alkenyl substituted with one or more R⁶. In some embodiments, R¹ is C₂-C₆-alkynyl substituted with one or more R⁶. In some embodiments, R¹ is C₁-C₆-heteroalkyl substituted with one or more R⁶. In some embodiments, R¹ is C₁-C₆-haloalkyl substituted with one or more R⁶. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆ alkylene-aryl (e.g., benzyl). In some embodiments, R¹ is C₁-C₆ alkenylene-aryl. In some embodiments, R¹ is C₁-C₆ alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆ alkylene-aryl, unsubstituted C₁-C₆ alkenylene-aryl, unsubstituted C₁-C₆ alkylene-heteroaryl, or unsubstituted heteroaryl. In some embodiments, R¹ is cycloalkyl substituted with one or more R⁶. In some embodiments, R¹ is heterocyclyl substituted with one or more R⁶. In some embodiments, R¹ is aryl substituted with one or more R⁶. In some embodiments, R¹ is C₁-C₆ alkylene-aryl substituted with one or more R⁶. In some embodiments, R¹ is C₁-C₆ alkenylene-aryl substituted with one or more R⁶. In some embodiments, R¹ is C₁-C₆ alkylene-heteroaryl substituted with one or more R⁶. In some embodiments, R¹ is heteroaryl substituted with one or more R⁶.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is-C(O)NR^{B}R^{C}. In some embodiments, R¹ is —C(O)R^{D}. In some embodiments, R¹ is —C(O)OR^{D}. In some embodiments, R¹ is —SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁶.

In some embodiments for Formulas (I) and (II), R² is hydrogen. In some embodiments, R² is halo (e.g., fluoro, chloro, bromo, or iodo). In some embodiments, R² is cyano. In some embodiments, R² is C₁-C₆-alkyl. In some embodiments, R² is C₂-C₆-alkenyl. In some embodiments, R² is C₂-C₆-alkynyl. In some embodiments, R² is —OR^{A} (e.g., —OH). In some embodiments, R^{3a}, R^{3b}, or both are independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}. In some embodiments, R^{3a} and R^{3b} are each independently hydrogen or C₁-C₆-alkyl. In some embodiments, R^{3a} is hydrogen. In some embodiments, R^{3b} is hydrogen. In some embodiments, R^{3a} is C₁-C₆-alkyl (e.g., methyl). In some embodiments, R^{3b} is C₁-C₆-alkyl (e.g., methyl). In some embodiments, R^{3a} is halo (e.g., fluoro, chloro, bromo, or iodo). In some embodiments, R^{3b} is halo (e.g., fluoro, chloro, bromo, or iodo). In some embodiments, R^{3a} is cyano. In some embodiments, R^{3b} is cyano. In some embodiments, R^{3a} is —OR^{A} (e.g., —OH). In some embodiments, R^{3b} is —OR^{A} (e.g., —OH). In some embodiments, R^{3a} is —NR^{B}R^{C}. In some embodiments, R^{3b} is —NR^{B}R^{C}. In some embodiments, R^{3a} is —C(O)R^{D}. In some embodiments, R^{3b} is —C(O)R^{D}. In some embodiments, R^{3a} is —C(O)OR^{D}. In some embodiments, R^{3b} is —C(O)OR^{D} In some embodiments, each of R^{3a} and R^{3b}, together with the carbon atom to which they are attached, form an oxo group.

In some embodiments, R^{3c} is hydrogen. In some embodiments, R^{3c} is C₁-C₆-alkyl. In some embodiments, R^{3c} is methyl. In some embodiments, R^{3c} is not hydrogen. In some embodiments, R^{3c} is not methyl. In some embodiments, R^{3c} is C₁-C₆ alkyl. In some embodiments, R^{3c} is C₁-C₆ substituted with one or more R′.

In some embodiments, R⁴ is hydrogen. In some embodiments, R⁴ is C₁-C₆ alkyl. In some embodiments, R⁴ is C₁-C₆ haloalkyl (e.g., —CF₃ or —CHF₂). In some embodiments, R⁴ is methyl.

In some embodiments, R⁵ is hydrogen. In some embodiments, R⁵ is C₁-C₆-alkyl. In some embodiments, R⁵ is C₁-C₆-heteroalkyl. In some embodiments, R⁵ is C₁-C₆-haloalkyl. In some embodiments, R⁵ is cycloalkyl. In some embodiments, R⁵ is halo (e.g., fluoro, chloro, bromo, or iodo). In some embodiments, R⁵ is cyano. In some embodiments, R⁵ is oxo. In some embodiments, R⁵ is —OR^{A}. In some embodiments, R⁵ is —NR^{B}R^{C}. In some embodiments, R⁵ is —C(O)R^{D} or —C(O)OR^{D}.

In some embodiments, R⁶ is C₁-C₆-alkyl. In some embodiments, R⁶ is C₂-C₆-alkenyl. In some embodiments, R⁶ is C₂-C₆-alkynyl. In some embodiments, R⁶ is C₁-C₆-heteroalkyl. In some embodiments, R⁶ is C₁-C₆-haloalkyl. In some embodiments, R⁶ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁶ is C₁-C₆-alkyl substituted with one or more R¹¹. In some embodiments, R⁶ is C₂-C₆-alkenyl substituted with one or more R¹¹. In some embodiments, R⁶ is C₂-C₆-alkynyl substituted with one or more R¹¹. In some embodiments, R⁶ is C₁-C₆-haloalkyl substituted with one or more R¹¹. In some embodiments, R⁶ is C₁-C₆-heteroalkyl substituted with one or more R¹¹.

In some embodiments, R⁶ is cycloalkyl. In some embodiments, R⁶ is heterocyclyl. In some embodiments, R⁶ is aryl. In some embodiments, R⁶ is heteroaryl. In some embodiments, R⁶ is unsubstituted cycloalkyl, unsubstituted heterocyclyl, unsubstituted aryl, or unsubstituted heteroaryl. In some embodiments, R⁶ is cycloalkyl substituted with one or more R¹¹. In some embodiments, R⁶ is heterocyclyl substituted with one or more R¹¹. In some embodiments, R⁶ is aryl substituted with one or more R¹¹. In some embodiments, R⁶ is heteroaryl substituted with one or more R¹¹.

In some embodiments, R⁶ is halo (e.g., fluoro, chloro, bromo, or iodo). In some embodiments, R⁶ is cyano. In some embodiments, R⁶ is oxo. In some embodiments, R⁶ is —OR^{A}. In some embodiments, R⁶ is —NR^{B}R^{C}. In some embodiments, R⁶ is —NR^{B}C(O)R^{D}. In some embodiments, R⁶ is —NO₂. In some embodiments, R⁶ is —C(O)NR^{B}R^{C}. In some embodiments, R⁶ is —C(O)R^{D}. In some embodiments, R⁶ is —C(O)OR^{D}. In some embodiments, R⁶ is —SR^{E}. In some embodiments, R⁶ is —S(O)ₓR^{D}.

In some embodiments, R⁷ is C₁-C₆-alkyl. In some embodiments, R⁷ is halo (e.g., fluoro, chloro, bromo, or iodo). In some embodiments, R⁷ is cyano. In some embodiments, R⁷ is oxo. In some embodiments, R⁷ is —OR^{A1} (e.g., —OH).

In some embodiments, R¹¹ is C₁-C₆-alkyl. In some embodiments, R¹¹ is C₁-C₆-heteroalkyl. In some embodiments, R¹¹ is C₁-C₆-haloalkyl (e.g., —CF₃). In some embodiments, R¹¹ is cycloalkyl. In some embodiments, R¹¹ is heterocyclyl. In some embodiments, R¹¹ is aryl. In some embodiments, R¹¹ is heteroaryl. In some embodiments, R¹¹ is halo. In some embodiments, R¹¹ is cyano. In some embodiments, R¹¹ is oxo. In some embodiments, R¹¹ is —OR^{A}.

In some embodiments for Formulas (I) and (II), R^{A} is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)ₓR^{D}.

In some embodiments, R^{B}, R^{C}, or both are independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R⁷.

In some embodiments, R^{D}, R^{E}, or both are independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments, each of R^{D} and R^{E} is independently hydrogen. In some embodiments, each of R^{D} and R^{E} is independently C₁-C₆ alkyl. In some embodiments, R^{D} is hydrogen. In some embodiments, R^{E} is hydrogen. In some embodiments, R^{D} is C₁-C₆ alkyl (e.g., methyl). In some embodiments, R^{E} is C₁-C₆ alkyl (e.g., methyl). In some embodiments, R^{D} is C₁-C₆ heteroalkyl. In some embodiments, R^{E} is C₁-C₆ heteroalkyl. In some embodiments, R^{D} is C₁-C₆ haloalkyl. In some embodiments, R^{E} is C₁-C₆ haloalkyl. In some embodiments, R^{D} is cycloalkyl. In some embodiments, R^{E} is cycloalkyl. In some embodiments, R^{D} is heterocyclyl. In some embodiments, R^{E} is heterocyclyl. In some embodiments, R^{D} is aryl. In some embodiments, R^{E} is aryl. In some embodiments, R^{D} is heteroaryl. In some embodiments, R^{E} is heteroaryl. In some embodiments, R^{D} is C₁-C₆ alkylene-aryl (e.g., benzyl). In some embodiments, R^{E} is C₁-C₆ alkylene-aryl (e.g., benzyl). In some embodiments, R^{D} is C₁-C₆ alkylene-heteroaryl. In some embodiments, R^{E} is C₁-C₆ alkylene-heteroaryl.

In some embodiments, R^{A1} is hydrogen. In some embodiments, R^{A1} is C₁-C₆-alkyl (e.g., methyl).

In some embodiments, m is 0, 1, or 2. In some embodiments, m is 0. In some embodiments, m is 1. In some embodiments, m is 2. In some embodiments, x is 0, 1, or 2. In some embodiments, x is 0. In some embodiments, x is 1. In some embodiments, x is 2. In some embodiments y is 0 or 1. In some embodiments, y is 0.

In some embodiments, the compound of Formula (I) is a compound of Formula (I-a):

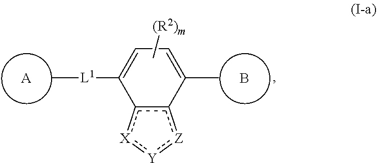

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; X, Y, and Z are each independently C(R^{3a}), C(R^{3a})(R^{3b}), N, N(R^{3c}), or 0, wherein at least one of X, Y, and Z is N, N(R^{3c}), or 0, and the bonds in the ring comprising X, Y, and Z may be single or double bonds as valency permits; L¹ is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-b):

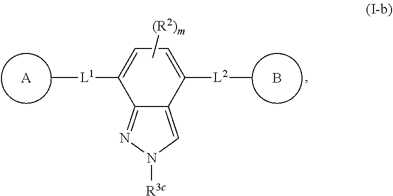

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E} or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-c):

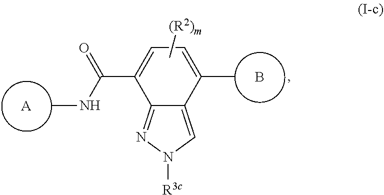

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-d):

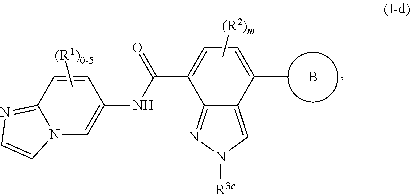

or a pharmaceutically acceptable salt, solvate, hydrate, tautomer, or stereoisomer thereof, wherein B is cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁—C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-e):

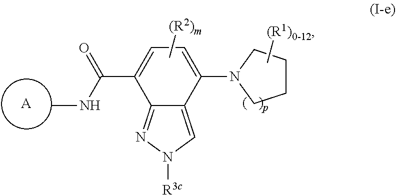

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; p is 0, 1, 2, or 3; and x is 0, 1, or 2.

In some embodiments, the compound of Formula (I) is a compound of Formula (I-f):

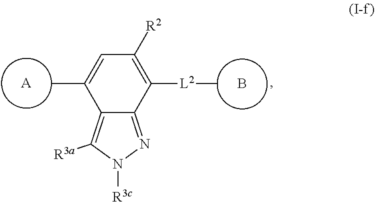

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L² is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —N(R^{B})(R^{C}), or —OR^{A}; R^{3a} is hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is independently C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; and x is 0, 1, or 2.

In some embodiments, R² is halo. In some embodiments, R² is fluoro. In some embodiments, R² is —OR^{A}. In some embodiments, R² is —N(R^{B})(R^{C}).

In some embodiments, the compound of Formula (I) is a compound of Formula (I-g):

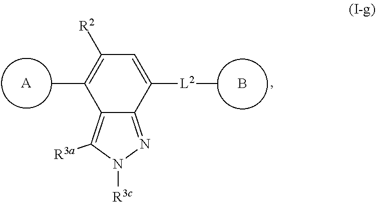

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L² is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —N(R^{B})(R^{C}), or —OR^{A}; R^{3a} is hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A}1; each R⁸ is independently C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; and x is 0, 1, or 2.

In some embodiments, the compound of Formula (I) is a compound of Formula (I-h):

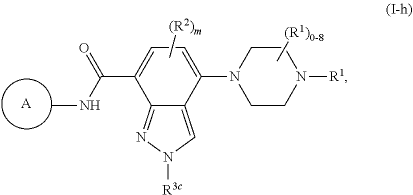

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L² is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —N(R^{B})(R^{C}), or —OR^{A}; R^{3a}, is hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is independently C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; and x is 0, 1, or 2.

In some embodiments of any one of Formula (I), (I-a), (I-b), (I-c), (I-d), (I-e), (I-f), and (I-g), R^{3c} is not hydrogen or methyl. In some embodiments, R^{3c} is not hydrogen. In some embodiments, R^{3c} is not methyl. In some embodiments, R^{3c} is not ethyl. In some embodiments, a compound of Formula (I) is not Compound 143, 207, 208, 209, 210, 211, 212, 228, 229, 230, 231, 234, 235, 237, 238, 239, 240, 241, 242, 243, 244, 245, 246, 247, 249, 250, 251, 252, 253, 258, 259, 260, 269, 270, 272, 273, 274, 275, 277, 278, 279, 280, 281, 284, 285, 286, or 287. In some embodiments, a compound of Formula (I) is not Compound 284.

In some embodiments, the compound of Formula (I) is selected from a compound in Table 1, or a pharmaceutically acceptable salt, solvate, hydrate, tautomer, or stereoisomer thereof.

TABLE 1
Exemplary compounds of Formula (I)

| Compound | |
|---|---|
| No. | Structure |
| 118 | 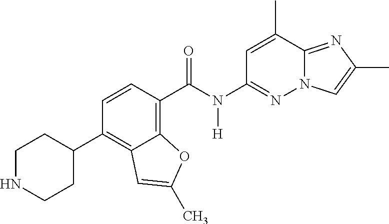 [SMILES: Cc1cn2nc(NC(=O)c3ccc(C4CCNCC4)c4cc(C)oc34)cc(C)c2n1] |
| 119 | 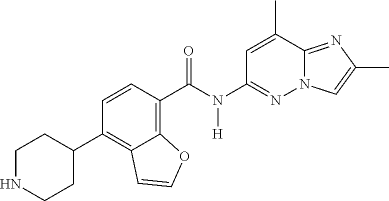 [SMILES: Cc1cn2nc(NC(=O)c3ccc(C4CCNCC4)c4ccoc34)cc(C)c2n1] |
| 140 | 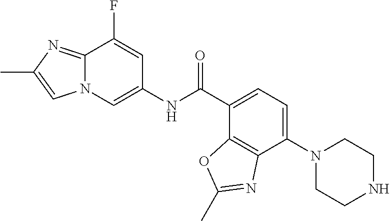 [SMILES: Cc1cn2cc(NC(=O)c3ccc(N4CCNCC4)c4nc(C)oc34)cc(F)c2n1] |
| 141 | 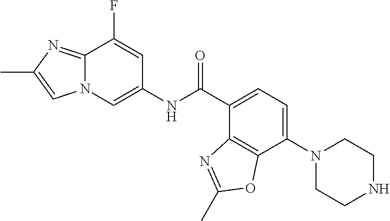 [SMILES: Cc1cn2cc(NC(=O)c3ccc(N4CCNCC4)c4oc(C)nc34)cc(F)c2n1] |
| 142 | 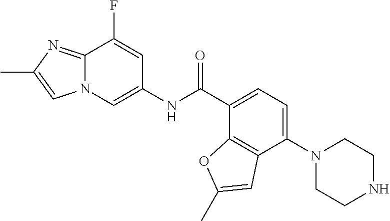 [SMILES: Cc1cn2cc(NC(=O)c3ccc(N4CCNCC4)c4cc(C)oc34)cc(F)c2n1] |
| 143 | 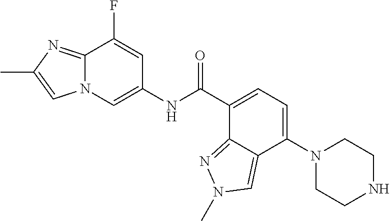 [SMILES: Cc1cn2cc(NC(=O)c3ccc(N4CCNCC4)c4cn(C)nc34)cc(F)c2n1] |
| 145 | 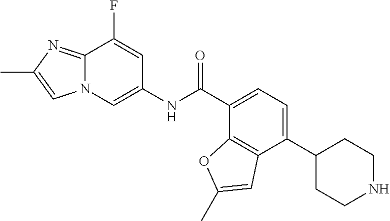 [SMILES: Cc1cn2cc(NC(=O)c3ccc(C4CCNCC4)c4cc(C)oc34)cc(F)c2n1] |
| 146 | 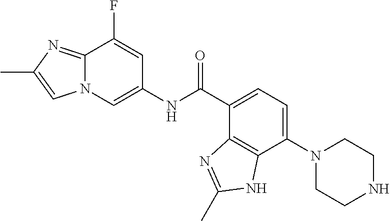 [SMILES: Cc1cn2cc(NC(=O)c3ccc(N4CCNCC4)c4[nH]c(C)nc34)cc(F)c2n1] |
| 147 | 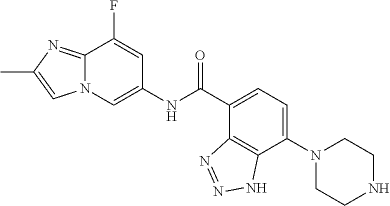 [SMILES: Cc1cn2cc(NC(=O)c3ccc(N4CCNCC4)c4[nH]nnc34)cc(F)c2n1] |
| 148 | 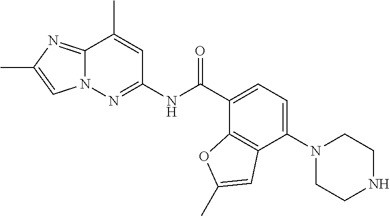 [SMILES: Cc1cn2nc(NC(=O)c3ccc(N4CCNCC4)c4cc(C)oc34)cc(C)c2n1] |
| 149 | 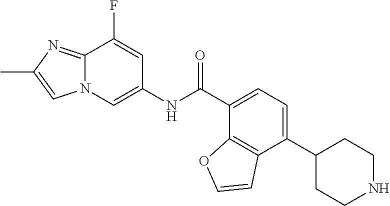 [SMILES: Cc1cn2cc(NC(=O)c3ccc(C4CCNCC4)c4ccoc34)cc(F)c2n1] |
| 150 | 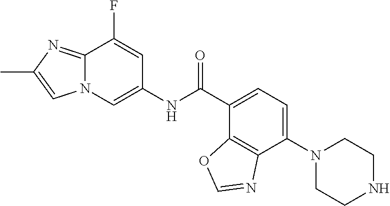 [SMILES: Cc1cn2cc(NC(=O)c3ccc(N4CCNCC4)c4ncoc34)cc(F)c2n1] |
| 187 | 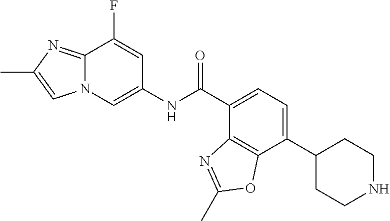 [SMILES: Cc1cn2cc(NC(=O)c3ccc(C4CCNCC4)c4oc(C)nc34)cc(F)c2n1] |
| 188 | 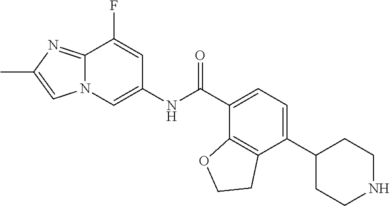 [SMILES: Cc1cn2cc(NC(=O)c3ccc(C4CCNCC4)c4c3OCC4)cc(F)c2n1] |
| 189 | 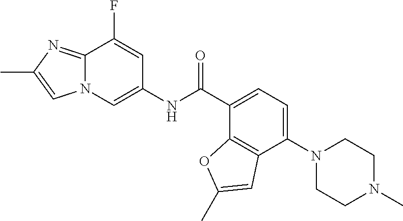 |
| 190 | 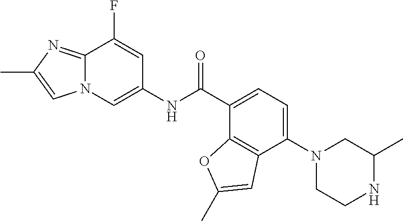 |
| 191 | 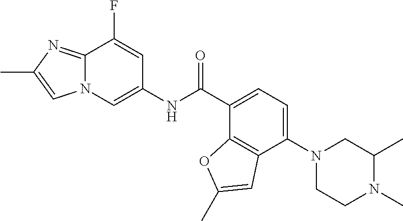 |
| 192 | 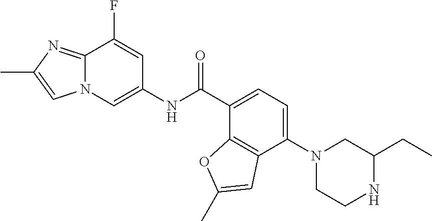 [SMILES: CCC1CN(c2ccc(C(=O)Nc3cc(F)c4nc(C)cn4c3)c3oc(C)cc23)CCN1] |
| 193 | 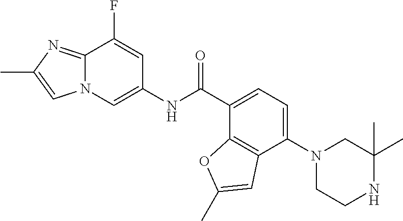 |
| 194 | 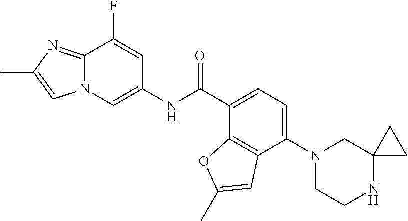 [SMILES: Cc1cn2cc(NC(=O)c3ccc(N4CCNC5(CC5)C4)c4cc(C)oc34)cc(F)c2n1] |
| 195 | 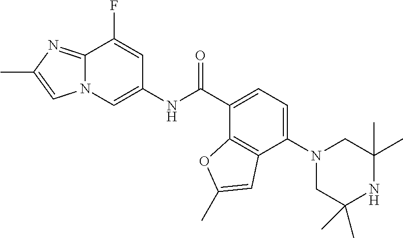 |
| 196 | 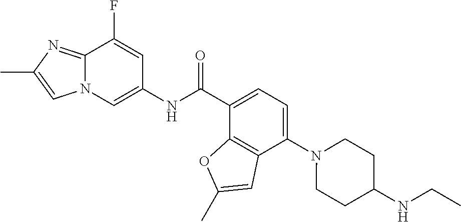 [SMILES: CCNC1CCN(c2ccc(C(=O)Nc3cc(F)c4nc(C)cn4c3)c3oc(C)cc23)CC1] |
| 197 | 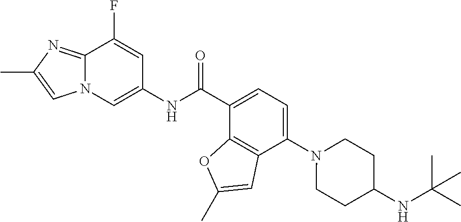 [SMILES: Cc1cn2cc(NC(=O)c3ccc(N4CCC(NC(C)(C)C)CC4)c4cc(C)oc34)cc(F)c2n1] |
| 198 | 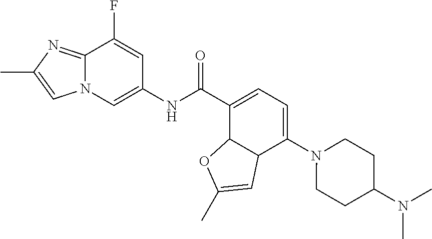 |
| 199 | 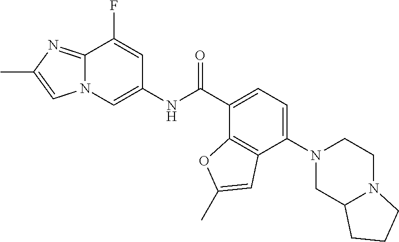 [SMILES: Cc1cn2cc(NC(=O)c3ccc(N4CCN5CCCC5C4)c4cc(C)oc34)cc(F)c2n1] |
| 200 | 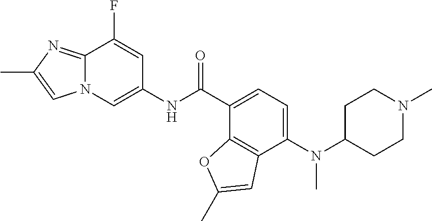 |
| 201 | 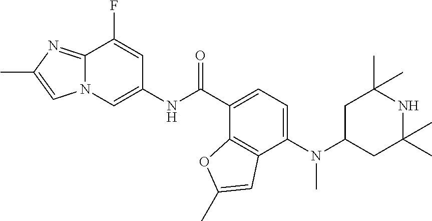 |
| 202 | 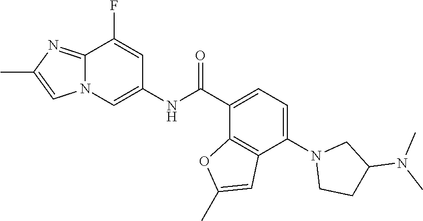 |
| 203 | 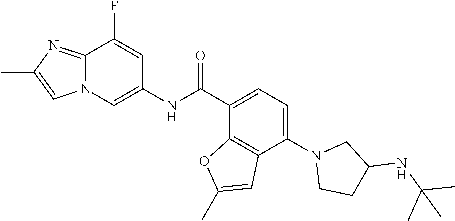 [SMILES: Cc1cn2cc(NC(=O)c3ccc(N4CCC(NC(C)(C)C)C4)c4cc(C)oc34)cc(F)c2n1] |
| 204 | 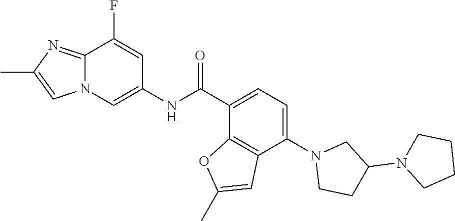 [SMILES: Cc1cn2cc(NC(=O)c3ccc(N4CCC(N5CCCC5)C4)c4cc(C)oc34)cc(F)c2n1] |
| 205 | 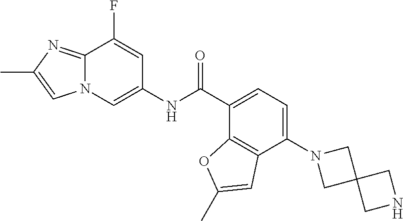 [SMILES: Cc1cn2cc(NC(=O)c3ccc(N4CC5(CNC5)C4)c4cc(C)oc34)cc(F)c2n1] |
| 206 | 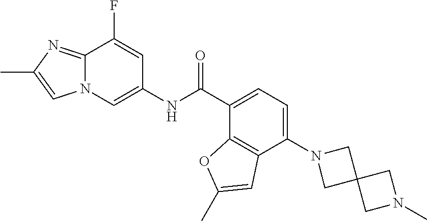 |
| 207 | 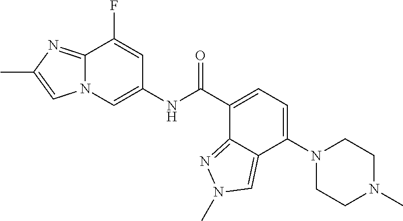 |
| 208 | 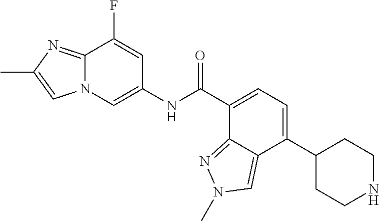 [SMILES: Cc1cn2cc(NC(=O)c3ccc(C4CCNCC4)c4cn(C)nc34)cc(F)c2n1] |
| 209 | 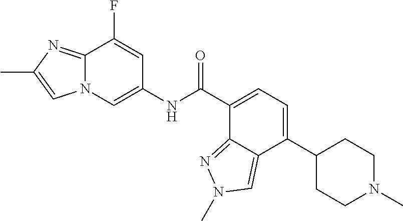 |
| 210 | 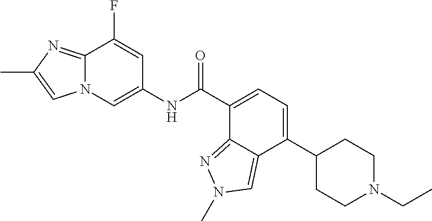 [SMILES: CCN1CCC(c2ccc(C(=O)Nc3cc(F)c4nc(C)cn4c3)c3nn(C)cc23)CC1] |
| 211 | 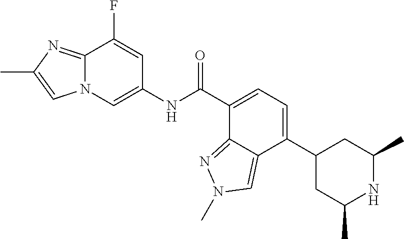 |
| 212 | 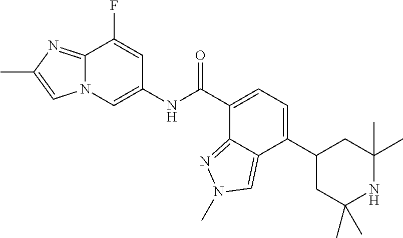 |
| 217 | 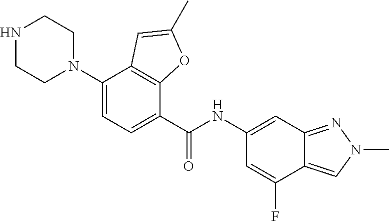 [SMILES: Cc1cc2c(N3CCNCC3)ccc(C(=O)Nc3cc(F)c4cn(C)nc4c3)c2o1] |
| 218 | 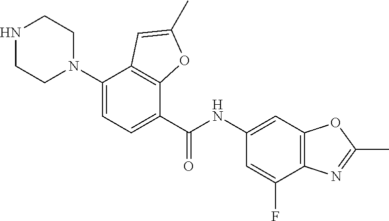 [SMILES: Cc1cc2c(N3CCNCC3)ccc(C(=O)Nc3cc(F)c4nc(C)oc4c3)c2o1] |
| 219 | 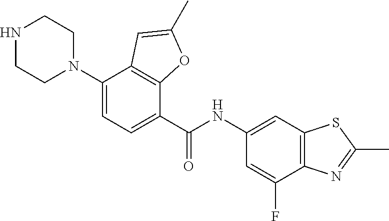 [SMILES: Cc1cc2c(N3CCNCC3)ccc(C(=O)Nc3cc(F)c4nc(C)sc4c3)c2o1] |
| 228 | 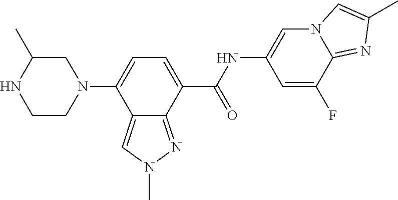 [SMILES: Cc1cn2cc(NC(=O)c3ccc(N4CCNC(C)C4)c4cn(C)nc34)cc(F)c2n1] |
| 229 | 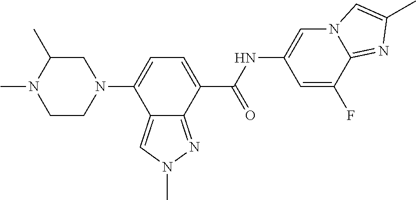 |
| 230 | 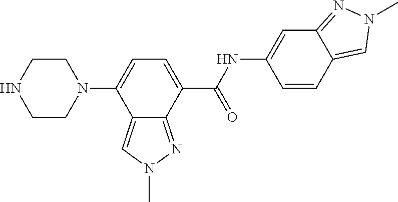 [SMILES: Cn1cc2ccc(NC(=O)c3ccc(N4CCNCC4)c4cn(C)nc34)cc2n1] |
| 231 | 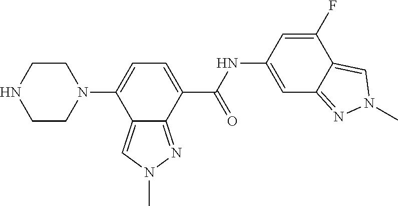 [SMILES: Cn1cc2c(F)cc(NC(=O)c3ccc(N4CCNCC4)c4cn(C)nc34)cc2n1] |
| 234 | 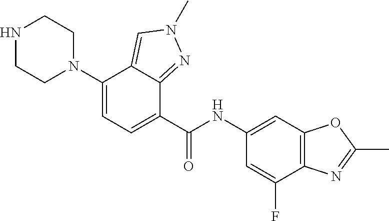 [SMILES: Cc1nc2c(F)cc(NC(=O)c3ccc(N4CCNCC4)c4cn(C)nc34)cc2o1] |
| 235 | 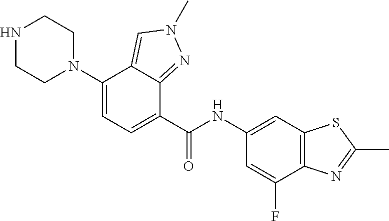 [SMILES: Cc1nc2c(F)cc(NC(=O)c3ccc(N4CCNCC4)c4cn(C)nc34)cc2s1] |
| 237 | 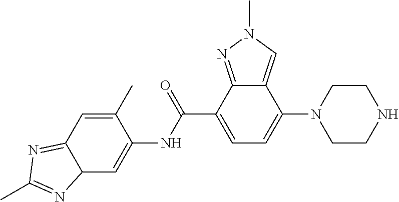 [SMILES: CC1=CC2=NC(C)=NC2C=C1NC(=O)c1ccc(N2CCNCC2)c2cn(C)nc12] |
| 238 | 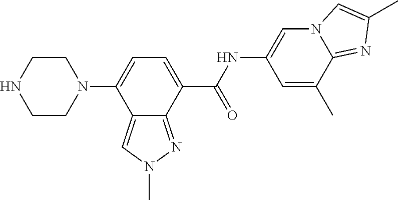 [SMILES: Cc1cn2cc(NC(=O)c3ccc(N4CCNCC4)c4cn(C)nc34)cc(C)c2n1] |
| 239 | 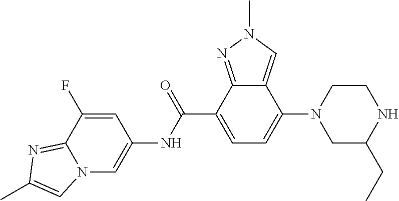 [SMILES: CCC1CN(c2ccc(C(=O)Nc3cc(F)c4nc(C)cn4c3)c3nn(C)cc23)CCN1] |
| 240 | 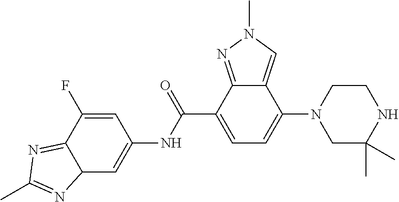 |
| 241 | 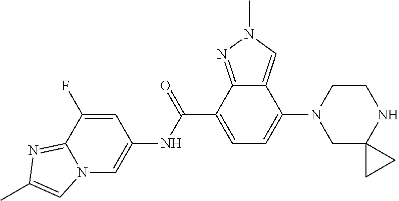 [SMILES: Cc1cn2cc(NC(=O)c3ccc(N4CCNC5(CC5)C4)c4cn(C)nc34)cc(F)c2n1] |
| 242 | 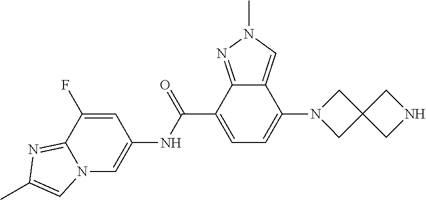 [SMILES: Cc1cn2cc(NC(=O)c3ccc(N4CC5(CNC5)C4)c4cn(C)nc34)cc(F)c2n1] |
| 243 | 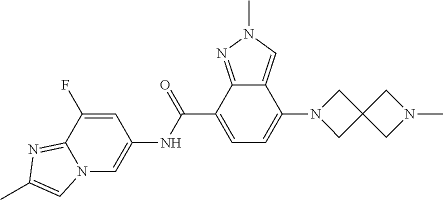 |
| 244 | 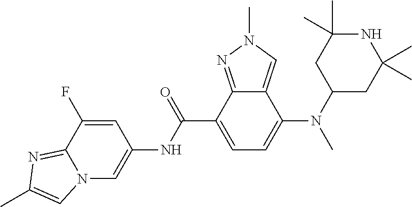 |
| 245 | 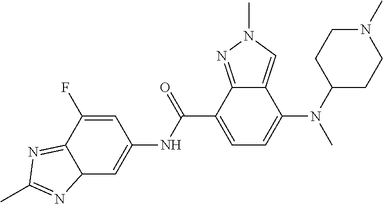 |
| 246 | 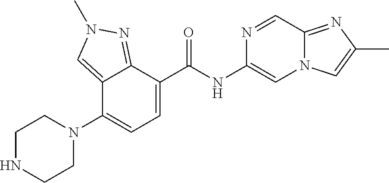 [SMILES: Cc1cn2cc(NC(=O)c3ccc(N4CCNCC4)c4cn(C)nc34)ncc2n1] |
| 247 | 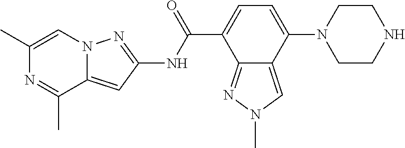 [SMILES: Cc1cn2nc(NC(=O)c3ccc(N4CCNCC4)c4cn(C)nc34)cc2c(C)n1] |
| 249 | 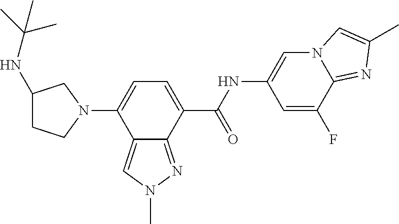 [SMILES: Cc1cn2cc(NC(=O)c3ccc(N4CCC(NC(C)(C)C)C4)c4cn(C)nc34)cc(F)c2n1] |
| 250 | 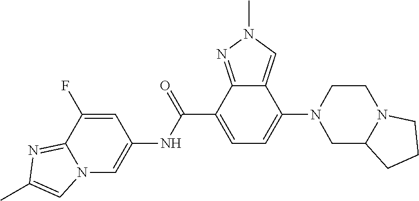 [SMILES: Cc1cn2cc(NC(=O)c3ccc(N4CCN5CCCC5C4)c4cn(C)nc34)cc(F)c2n1] |
| 251 | 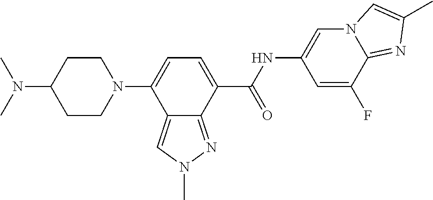 |
| 252 | 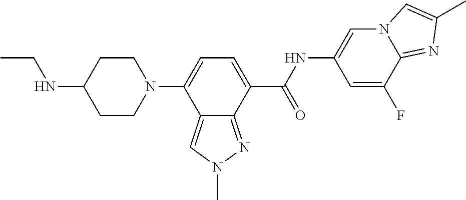 [SMILES: CCNC1CCN(c2ccc(C(=O)Nc3cc(F)c4nc(C)cn4c3)c3nn(C)cc23)CC1] |
| 253 | 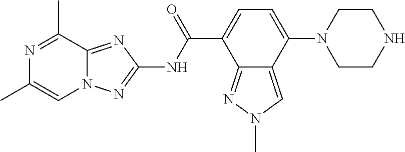 [SMILES: Cc1cn2nc(NC(=O)c3ccc(N4CCNCC4)c4cn(C)nc34)nc2c(C)n1] |
| 255 | 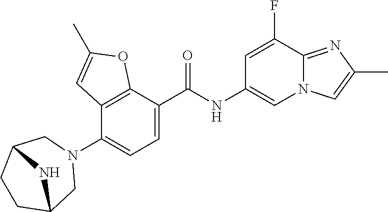 [SMILES: Cc1cn2cc(NC(=O)c3ccc(N4C[C@H]5CC[C@@H](C4)N5)c4cc(C)oc34)cc(F)c2n1] |
| 256 | 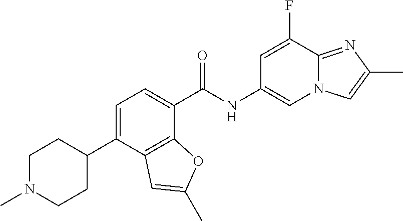 |
| 257 | 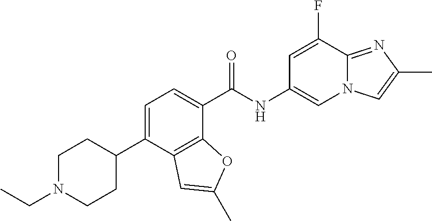 [SMILES: CCN1CCC(c2ccc(C(=O)Nc3cc(F)c4nc(C)cn4c3)c3oc(C)cc23)CC1] |
| 258 | 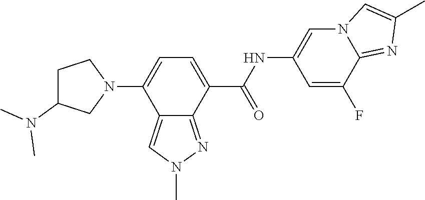 |
| 259 | 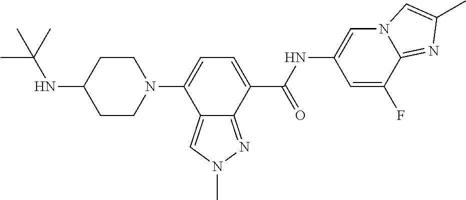 [SMILES: Cc1cn2cc(NC(=O)c3ccc(N4CCC(NC(C)(C)C)CC4)c4cn(C)nc34)cc(F)c2n1] |
| 260 | 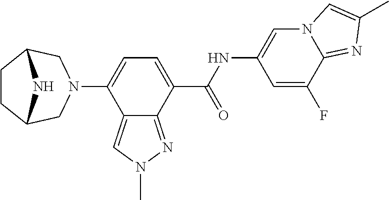 [SMILES: Cc1cn2cc(NC(=O)c3ccc(N4C[C@H]5CC[C@@H](C4)N5)c4cn(C)nc34)cc(F)c2n1] |
| 261 | 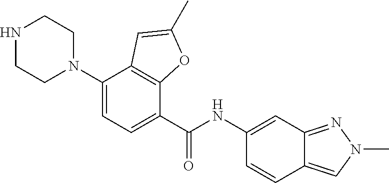 [SMILES: Cc1cc2c(N3CCNCC3)ccc(C(=O)Nc3ccc4cn(C)nc4c3)c2o1] |
| 262 | 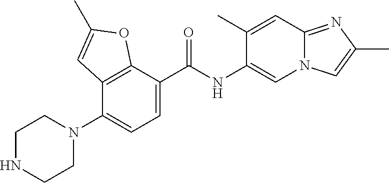 [SMILES: Cc1cn2cc(NC(=O)c3ccc(N4CCNCC4)c4cc(C)oc34)c(C)cc2n1] |
| 263 | 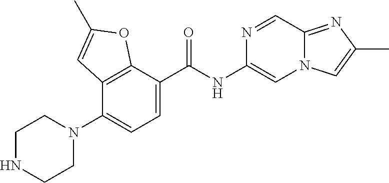 [SMILES: Cc1cn2cc(NC(=O)c3ccc(N4CCNCC4)c4cc(C)oc34)ncc2n1] |
| 264 | 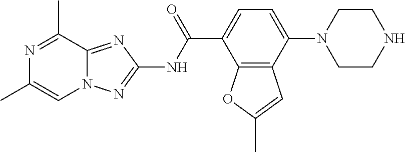 [SMILES: Cc1cn2nc(NC(=O)c3ccc(N4CCNCC4)c4cc(C)oc34)nc2c(C)n1] |
| 265 | 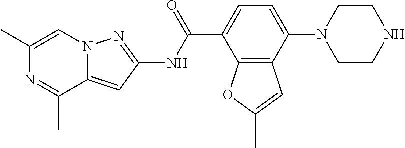 [SMILES: Cc1cn2nc(NC(=O)c3ccc(N4CCNCC4)c4cc(C)oc34)cc2c(C)n1] |
| 266 | 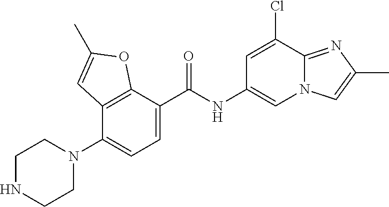 [SMILES: Cc1cn2cc(NC(=O)c3ccc(N4CCNCC4)c4cc(C)oc34)cc(Cl)c2n1] |
| 267 | 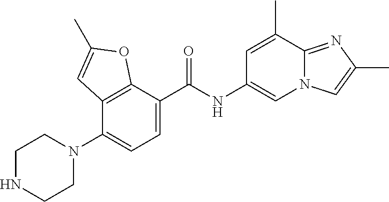 [SMILES: Cc1cn2cc(NC(=O)c3ccc(N4CCNCC4)c4cc(C)oc34)cc(C)c2n1] |
| 268 | 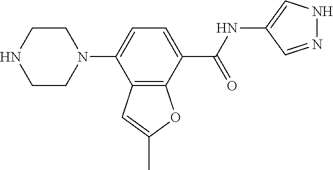 [SMILES: Cc1cc2c(N3CCNCC3)ccc(C(=O)Nc3cn[nH]c3)c2o1] |
| 269 | 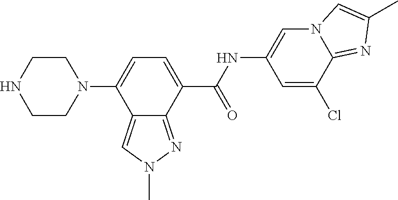 [SMILES: Cc1cn2cc(NC(=O)c3ccc(N4CCNCC4)c4cn(C)nc34)cc(Cl)c2n1] |
| 270 | 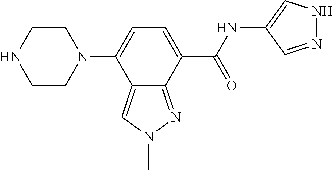 [SMILES: Cn1cc2c(N3CCNCC3)ccc(C(=O)Nc3cn[nH]c3)c2n1] |
| 271 | 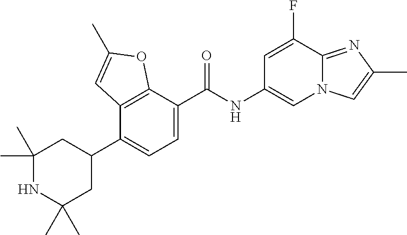 |
| 272 | 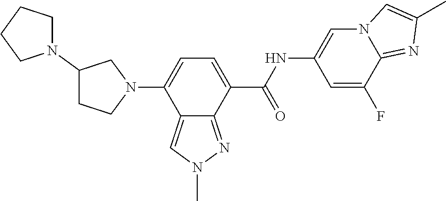 [SMILES: Cc1cn2cc(NC(=O)c3ccc(N4CCC(N5CCCC5)C4)c4cn(C)nc34)cc(F)c2n1] |
| 273 | 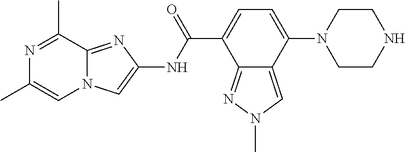 [SMILES: Cc1cn2cc(NC(=O)c3ccc(N4CCNCC4)c4cn(C)nc34)nc2c(C)n1] |
| 274 | 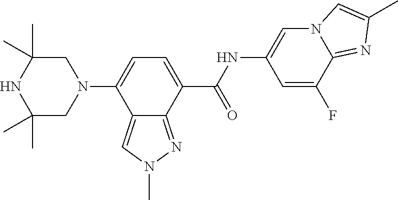 |
| 275 | 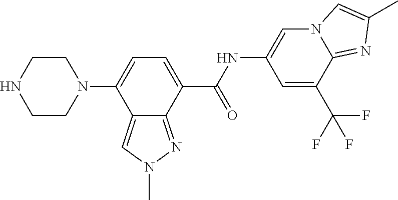 [SMILES: Cc1cn2cc(NC(=O)c3ccc(N4CCNCC4)c4cn(C)nc34)cc(C(F)(F)F)c2n1] |
| 277 | 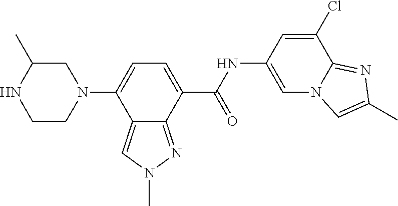 [SMILES: Cc1cn2cc(NC(=O)c3ccc(N4CCNC(C)C4)c4cn(C)nc34)cc(Cl)c2n1] |
| 278 | 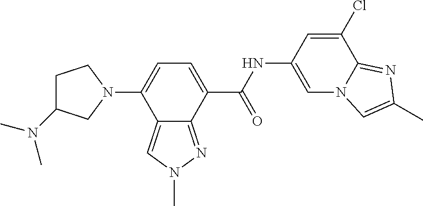 |
| 279 | 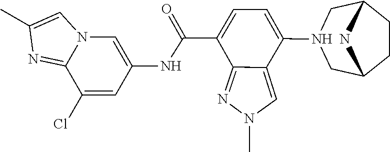 [SMILES: Cc1cn2cc(NC(=O)c3ccc(N4C[C@H]5CC[C@@H](C4)N5)c4cn(C)nc34)cc(Cl)c2n1] |
| 280 | 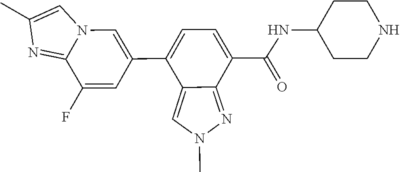 [SMILES: Cc1cn2cc(-c3ccc(C(=O)NC4CCNCC4)c4nn(C)cc34)cc(F)c2n1] |
| 281 | 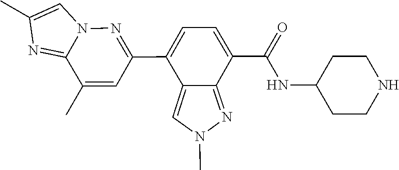 [SMILES: Cc1cn2nc(-c3ccc(C(=O)NC4CCNCC4)c4nn(C)cc34)cc(C)c2n1] |
| 282 | 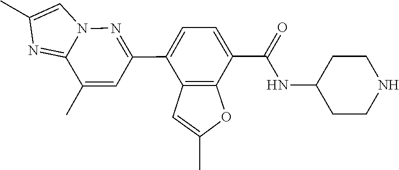 [SMILES: Cc1cn2nc(-c3ccc(C(=O)NC4CCNCC4)c4oc(C)cc34)cc(C)c2n1] |
| 283 | 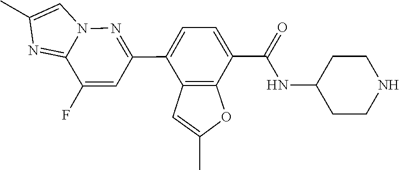 [SMILES: Cc1cn2nc(-c3ccc(C(=O)NC4CCNCC4)c4oc(C)cc34)cc(F)c2n1] |
| 284 | 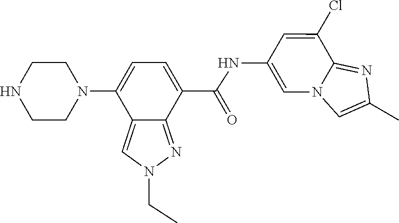 [SMILES: CCn1cc2c(N3CCNCC3)ccc(C(=O)Nc3cc(Cl)c4nc(C)cn4c3)c2n1] |
| 285 | 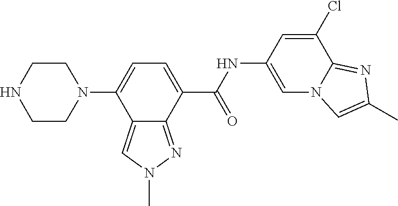 [SMILES: Cc1cn2cc(NC(=O)c3ccc(N4CCNCC4)c4cn(C)nc34)cc(Cl)c2n1] |
| 286 | 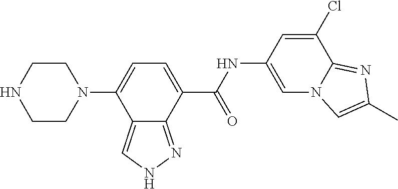 [SMILES: Cc1cn2cc(NC(=O)c3ccc(N4CCNCC4)c4c[nH]nc34)cc(Cl)c2n1] |
| 287 | 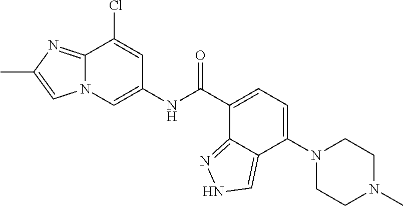 |
| 288 | 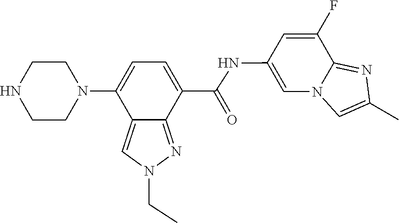 [SMILES: CCn1cc2c(N3CCNCC3)ccc(C(=O)Nc3cc(F)c4nc(C)cn4c3)c2n1] |
| 289 | 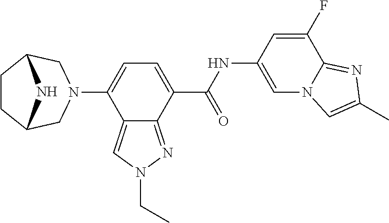 [SMILES: CCn1cc2c(N3C[C@H]4CC[C@@H](C3)N4)ccc(C(=O)Nc3cc(F)c4nc(C)cn4c3)c2n1] |
| 290 | 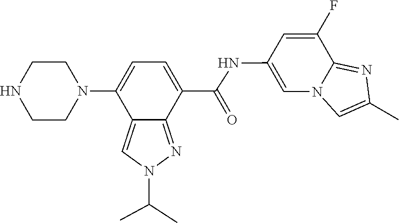 [SMILES: Cc1cn2cc(NC(=O)c3ccc(N4CCNCC4)c4cn(C(C)C)nc34)cc(F)c2n1] |
| 291 | 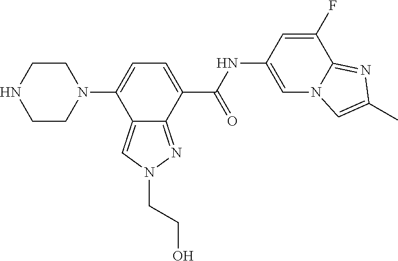 [SMILES: Cc1cn2cc(NC(=O)c3ccc(N4CCNCC4)c4cn(CCO)nc34)cc(F)c2n1] |
| 292 | 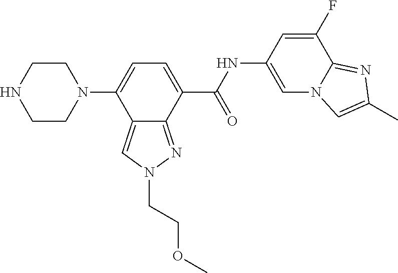 [SMILES: COCCn1cc2c(N3CCNCC3)ccc(C(=O)Nc3cc(F)c4nc(C)cn4c3)c2n1] |
| 293 | 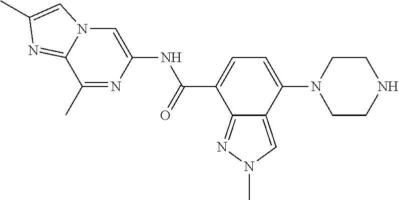 [SMILES: Cc1cn2cc(NC(=O)c3ccc(N4CCNCC4)c4cn(C)nc34)nc(C)c2n1] |
| 294 | 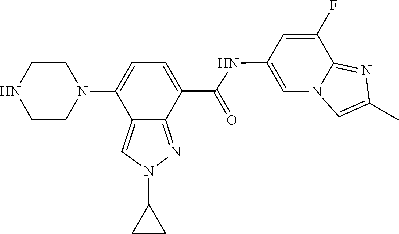 [SMILES: Cc1cn2cc(NC(=O)c3ccc(N4CCNCC4)c4cn(C5CC5)nc34)cc(F)c2n1] |
| 295 | 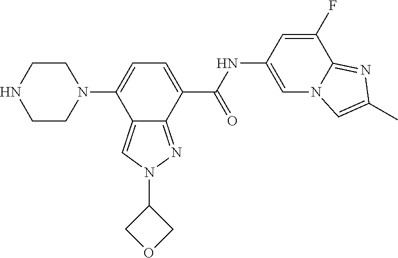 [SMILES: Cc1cn2cc(NC(=O)c3ccc(N4CCNCC4)c4cn(C5COC5)nc34)cc(F)c2n1] |
| 296 | 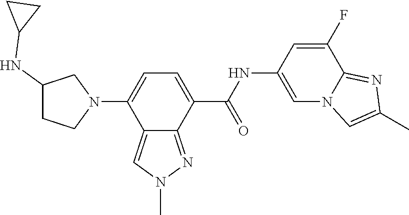 [SMILES: Cc1cn2cc(NC(=O)c3ccc(N4CCC(NC5CC5)C4)c4cn(C)nc34)cc(F)c2n1] |
| 297 | 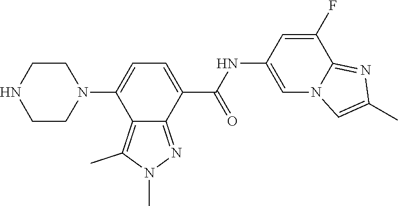 [SMILES: Cc1cn2cc(NC(=O)c3ccc(N4CCNCC4)c4c(C)n(C)nc34)cc(F)c2n1] |
| 298 | 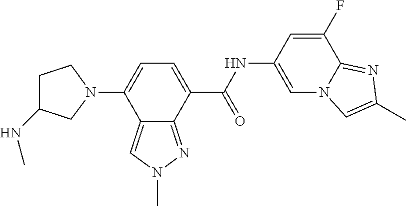 [SMILES: CNC1CCN(c2ccc(C(=O)Nc3cc(F)c4nc(C)cn4c3)c3nn(C)cc23)C1] |
| 299 | 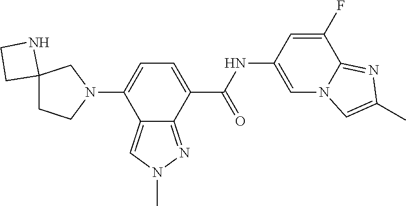 [SMILES: Cc1cn2cc(NC(=O)c3ccc(N4CCC5(CCN5)C4)c4cn(C)nc34)cc(F)c2n1] |
| 300 | 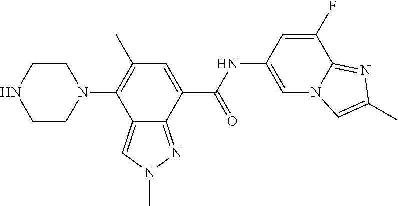 [SMILES: Cc1cn2cc(NC(=O)c3cc(C)c(N4CCNCC4)c4cn(C)nc34)cc(F)c2n1] |
| 301 | 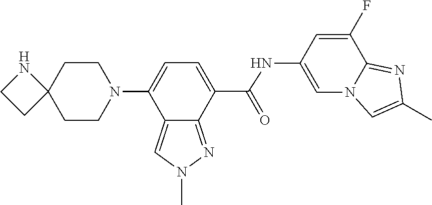 [SMILES: Cc1cn2cc(NC(=O)c3ccc(N4CCC5(CCN5)CC4)c4cn(C)nc34)cc(F)c2n1] |
| 302 | 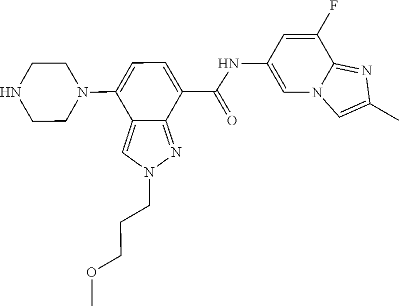 [SMILES: COCCCn1cc2c(N3CCNCC3)ccc(C(=O)Nc3cc(F)c4nc(C)cn4c3)c2n1] |
| 303 | 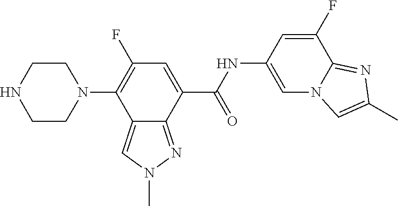 [SMILES: Cc1cn2cc(NC(=O)c3cc(F)c(N4CCNCC4)c4cn(C)nc34)cc(F)c2n1] |
| 304 | 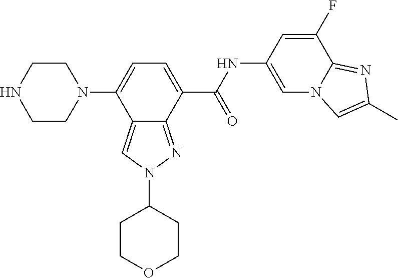 [SMILES: Cc1cn2cc(NC(=O)c3ccc(N4CCNCC4)c4cn(C5CCOCC5)nc34)cc(F)c2n1] |
| 305 | 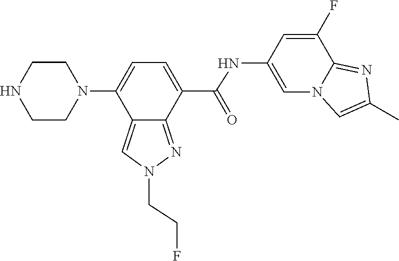 [SMILES: Cc1cn2cc(NC(=O)c3ccc(N4CCNCC4)c4cn(CCF)nc34)cc(F)c2n1] |
| 306 | 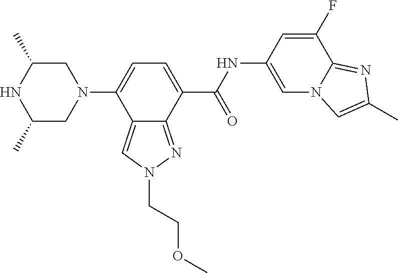 [SMILES: COCCn1cc2c(N3C[C@@H](C)N[C@@H](C)C3)ccc(C(=O)Nc3cc(F)c4nc(C)cn4c3)c2n1] |
| 307 | 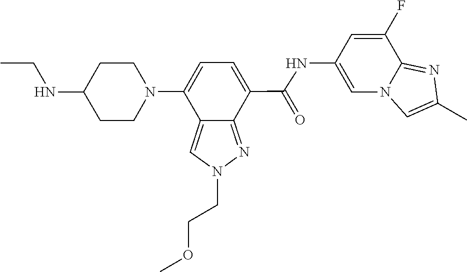 [SMILES: CCNC1CCN(c2ccc(C(=O)Nc3cc(F)c4nc(C)cn4c3)c3nn(CCOC)cc23)CC1] |
| 308 | 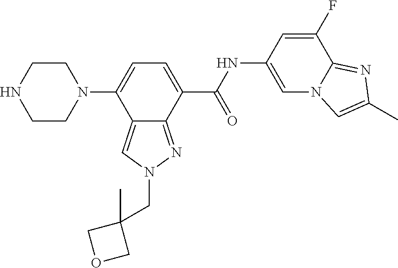 [SMILES: Cc1cn2cc(NC(=O)c3ccc(N4CCNCC4)c4cn(CC5(C)COC5)nc34)cc(F)c2n1] |
| 309 | 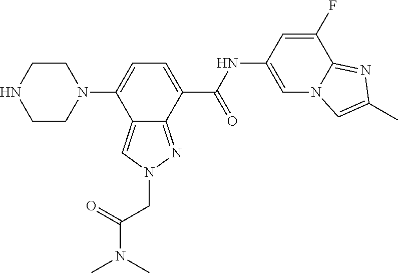 [SMILES: Cc1cn2cc(NC(=O)c3ccc(N4CCNCC4)c4cn(CC(=O)N(C)C)nc34)cc(F)c2n1] |
| 310 | 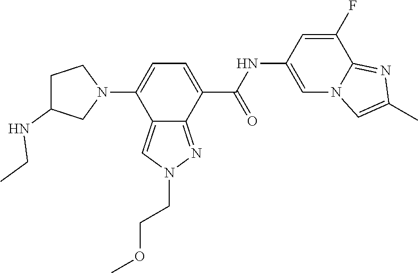 [SMILES: CCNC1CCN(c2ccc(C(=O)Nc3cc(F)c4nc(C)cn4c3)c3nn(CCOC)cc23)C1] |
| 311 | 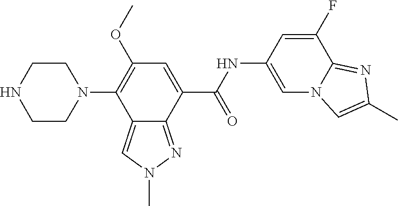 [SMILES: COc1cc(C(=O)Nc2cc(F)c3nc(C)cn3c2)c2nn(C)cc2c1N1CCNCC1] |
| 312 | 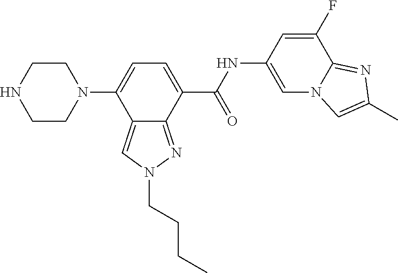 [SMILES: CCCCn1cc2c(N3CCNCC3)ccc(C(=O)Nc3cc(F)c4nc(C)cn4c3)c2n1] |
| 313 | 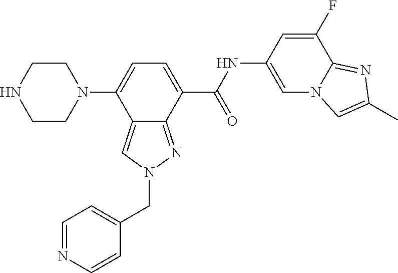 [SMILES: Cc1cn2cc(NC(=O)c3ccc(N4CCNCC4)c4cn(Cc5ccncc5)nc34)cc(F)c2n1] |
| 314 | 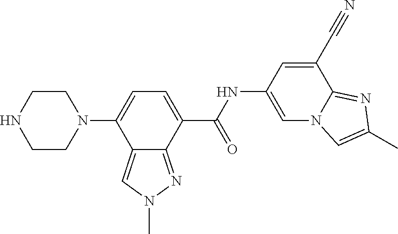 [SMILES: Cc1cn2cc(NC(=O)c3ccc(N4CCNCC4)c4cn(C)nc34)cc(C#N)c2n1] |
| 315 | 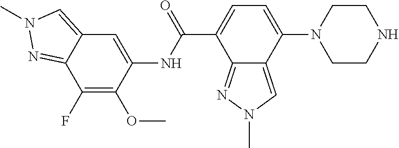 [SMILES: COc1c(NC(=O)c2ccc(N3CCNCC3)c3cn(C)nc23)cc2cn(C)nc2c1F] |
| 316 | 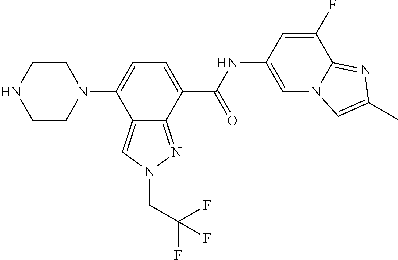 [SMILES: Cc1cn2cc(NC(=O)c3ccc(N4CCNCC4)c4cn(CC(F)(F)F)nc34)cc(F)c2n1] |
| 317 | 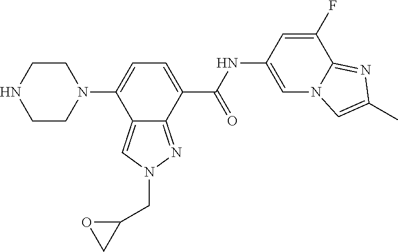 [SMILES: Cc1cn2cc(NC(=O)c3ccc(N4CCNCC4)c4cn(CC5CO5)nc34)cc(F)c2n1] |
| 318 | 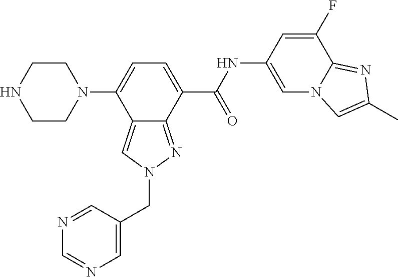 [SMILES: Cc1cn2cc(NC(=O)c3ccc(N4CCNCC4)c4cn(Cc5cncnc5)nc34)cc(F)c2n1] |
| 319 | 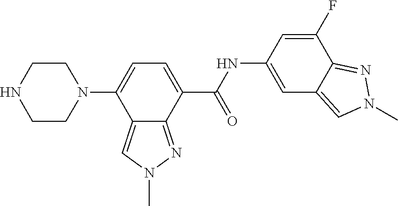 [SMILES: Cn1cc2cc(NC(=O)c3ccc(N4CCNCC4)c4cn(C)nc34)cc(F)c2n1] |
| 320 | 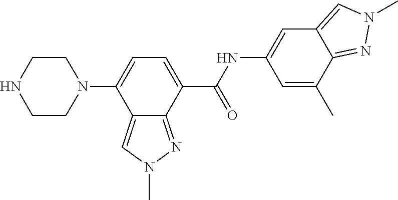 [SMILES: Cc1cc(NC(=O)c2ccc(N3CCNCC3)c3cn(C)nc23)cc2cn(C)nc12] |
| 321 | 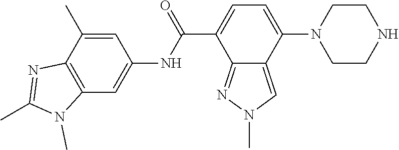 [SMILES: Cc1cc(NC(=O)c2ccc(N3CCNCC3)c3cn(C)nc23)cc2c1nc(C)n2C] |
| 322 | 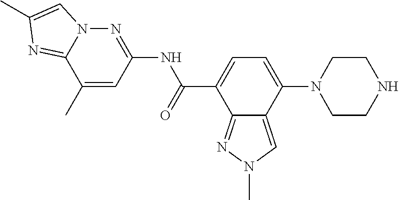 [SMILES: Cc1cn2nc(NC(=O)c3ccc(N4CCNCC4)c4cn(C)nc34)cc(C)c2n1] |
| 323 | 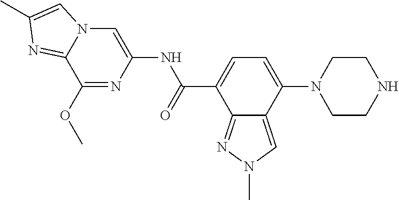 [SMILES: COc1nc(NC(=O)c2ccc(N3CCNCC3)c3cn(C)nc23)cn2cc(C)nc12] |
| 324 | 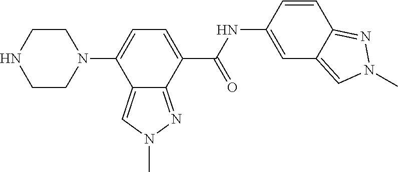 [SMILES: Cn1cc2cc(NC(=O)c3ccc(N4CCNCC4)c4cn(C)nc34)ccc2n1] |
| 325 | 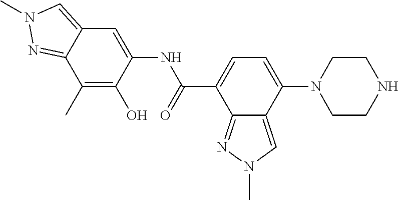 [SMILES: Cc1c(O)c(NC(=O)c2ccc(N3CCNCC3)c3cn(C)nc23)cc2cn(C)nc12] |
| 326 | 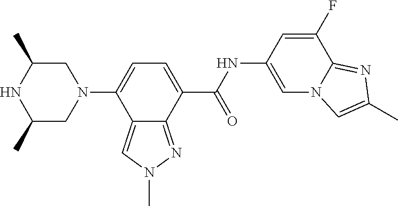 [SMILES: Cc1cn2cc(NC(=O)c3ccc(N4C[C@@H](C)N[C@@H](C)C4)c4cn(C)nc34)cc(F)c2n1] |
| 327 | 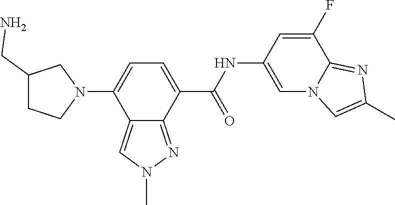 [SMILES: Cc1cn2cc(NC(=O)c3ccc(N4CCC(CN)C4)c4cn(C)nc34)cc(F)c2n1] |
| 328 | 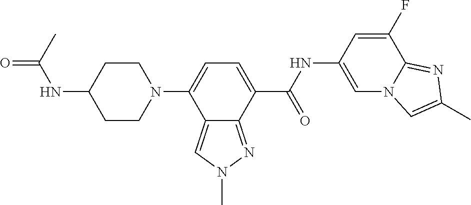 [SMILES: CC(=O)NC1CCN(c2ccc(C(=O)Nc3cc(F)c4nc(C)cn4c3)c3nn(C)cc23)CC1] |
| 329 | 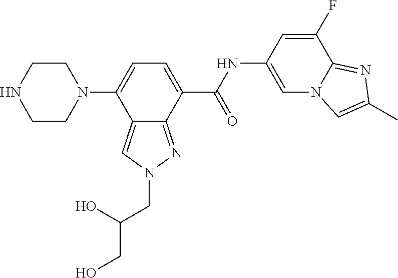 [SMILES: Cc1cn2cc(NC(=O)c3ccc(N4CCNCC4)c4cn(CC(O)CO)nc34)cc(F)c2n1] |
| 330 | 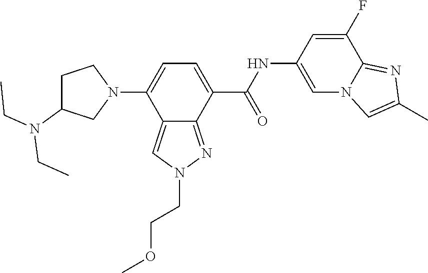 [SMILES: CCN(CC)C1CCN(c2ccc(C(=O)Nc3cc(F)c4nc(C)cn4c3)c3nn(CCOC)cc23)C1] |
| 331 | 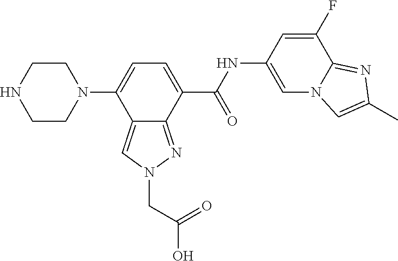 [SMILES: Cc1cn2cc(NC(=O)c3ccc(N4CCNCC4)c4cn(CC(=O)O)nc34)cc(F)c2n1] |
| 332 | 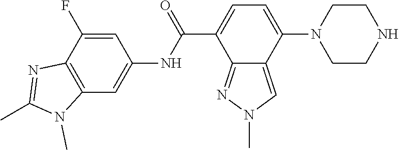 [SMILES: Cc1nc2c(F)cc(NC(=O)c3ccc(N4CCNCC4)c4cn(C)nc34)cc2n1C] |
| 333 | 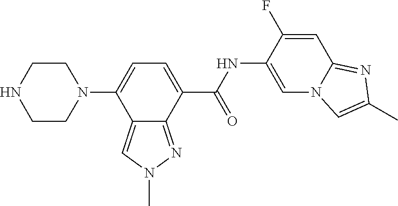 [SMILES: Cc1cn2cc(NC(=O)c3ccc(N4CCNCC4)c4cn(C)nc34)c(F)cc2n1] |
| 334 | 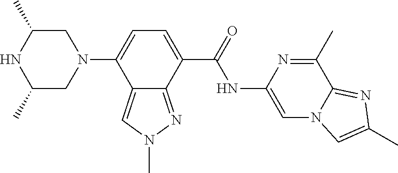 [SMILES: Cc1cn2cc(NC(=O)c3ccc(N4C[C@@H](C)N[C@@H](C)C4)c4cn(C)nc34)nc(C)c2n1] |
| 335 | 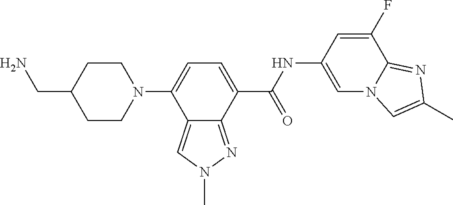 [SMILES: Cc1cn2cc(NC(=O)c3ccc(N4CCC(CN)CC4)c4cn(C)nc34)cc(F)c2n1] |
| 336 | 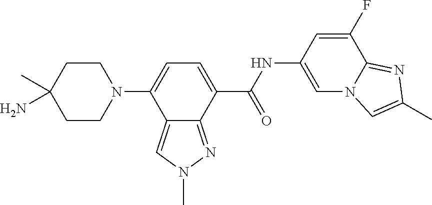 |
| 337 | 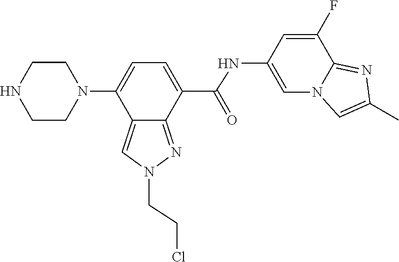 [SMILES: Cc1cn2cc(NC(=O)c3ccc(N4CCNCC4)c4cn(CCCl)nc34)cc(F)c2n1] |
| 338 | 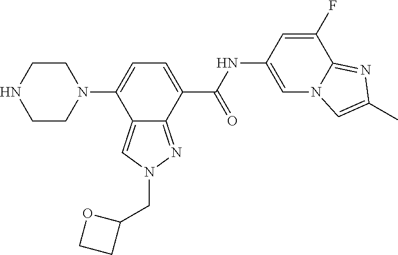 [SMILES: Cc1cn2cc(NC(=O)c3ccc(N4CCNCC4)c4cn(CC5CCO5)nc34)cc(F)c2n1] |
| 339 | 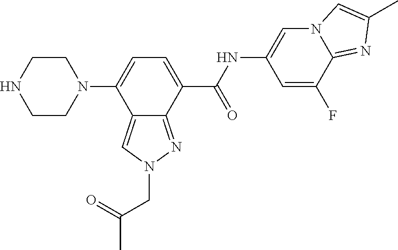 [SMILES: CC(=O)Cn1cc2c(N3CCNCC3)ccc(C(=O)Nc3cc(F)c4nc(C)cn4c3)c2n1] |
| 340 | 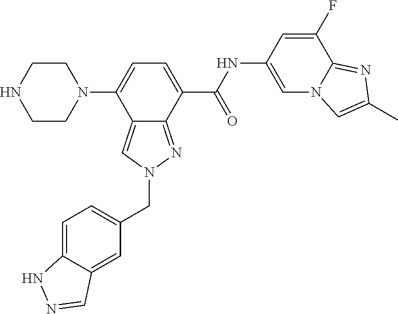 [SMILES: Cc1cn2cc(NC(=O)c3ccc(N4CCNCC4)c4cn(Cc5ccc6[nH]ncc6c5)nc34)cc(F)c2n1] |
| 341 | 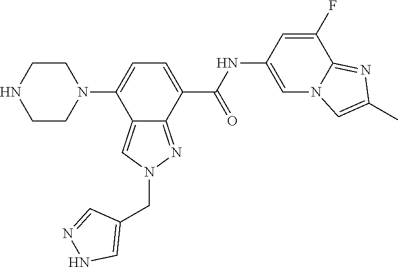 [SMILES: Cc1cn2cc(NC(=O)c3ccc(N4CCNCC4)c4cn(Cc5cn[nH]c5)nc34)cc(F)c2n1] |
| 342 | 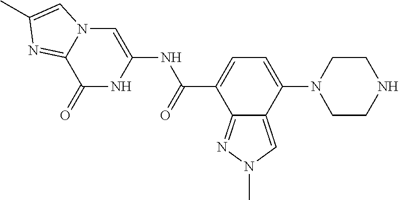 [SMILES: Cc1cn2cc(NC(=O)c3ccc(N4CCNCC4)c4cn(C)nc34)[nH]c(=O)c2n1] |
| 343 | 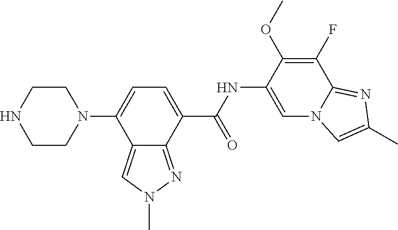 [SMILES: COc1c(NC(=O)c2ccc(N3CCNCC3)c3cn(C)nc23)cn2cc(C)nc2c1F] |
| 344 | 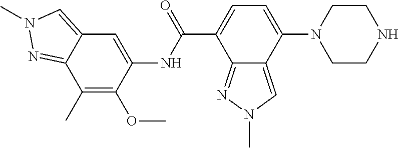 [SMILES: COc1c(NC(=O)c2ccc(N3CCNCC3)c3cn(C)nc23)cc2cn(C)nc2c1C] |
| 345 | 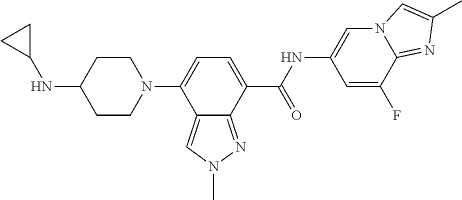 [SMILES: Cc1cn2cc(NC(=O)c3ccc(N4CCC(NC5CC5)CC4)c4cn(C)nc34)cc(F)c2n1] |
| 346 | 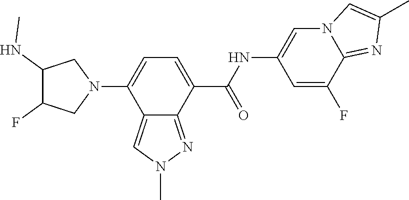 [SMILES: CNC1CN(c2ccc(C(=O)Nc3cc(F)c4nc(C)cn4c3)c3nn(C)cc23)CC1F] |
| 347 | 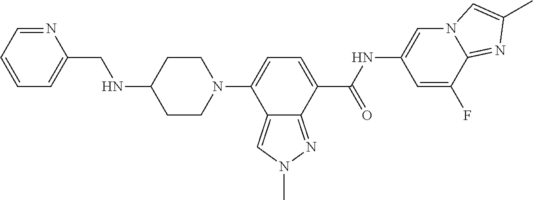 [SMILES: Cc1cn2cc(NC(=O)c3ccc(N4CCC(NCc5ccccn5)CC4)c4cn(C)nc34)cc(F)c2n1] |
| 348 | 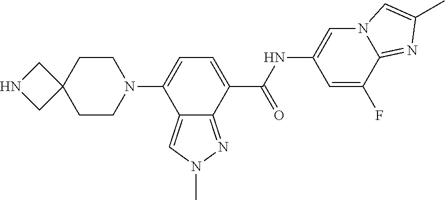 [SMILES: Cc1cn2cc(NC(=O)c3ccc(N4CCC5(CC4)CNC5)c4cn(C)nc34)cc(F)c2n1] |
| 349 | 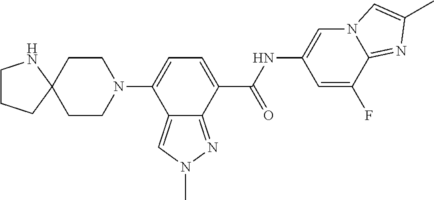 [SMILES: Cc1cn2cc(NC(=O)c3ccc(N4CCC5(CCCN5)CC4)c4cn(C)nc34)cc(F)c2n1] |
| 350 | 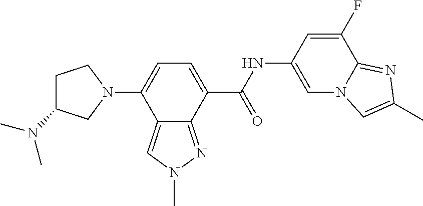 |
| 351 | 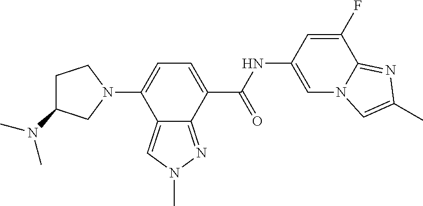 |
| 352 | 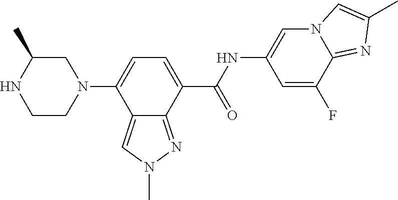 [SMILES: Cc1cn2cc(NC(=O)c3ccc(N4CCN[C@@H](C)C4)c4cn(C)nc34)cc(F)c2n1] |
| 353 | 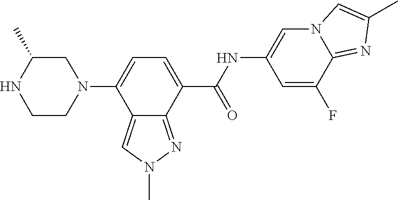 [SMILES: Cc1cn2cc(NC(=O)c3ccc(N4CCN[C@H](C)C4)c4cn(C)nc34)cc(F)c2n1] |
| 354 | 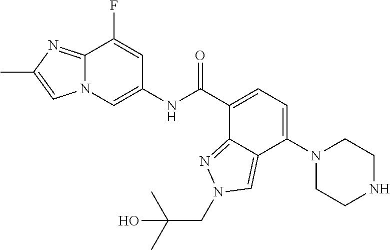 [SMILES: Cc1cn2cc(NC(=O)c3ccc(N4CCNCC4)c4cn(CC(C)(C)O)nc34)cc(F)c2n1] |
| 355 | 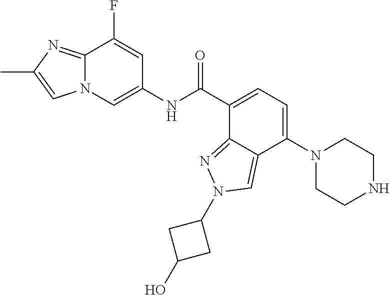 [SMILES: Cc1cn2cc(NC(=O)c3ccc(N4CCNCC4)c4cn(C5CC(O)C5)nc34)cc(F)c2n1] |
| 356 | 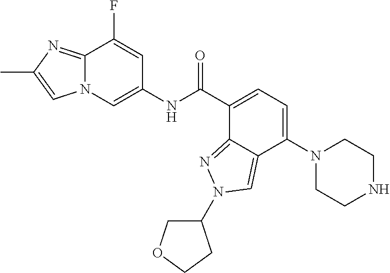 [SMILES: Cc1cn2cc(NC(=O)c3ccc(N4CCNCC4)c4cn(C5CCOC5)nc34)cc(F)c2n1] |
| 357 | 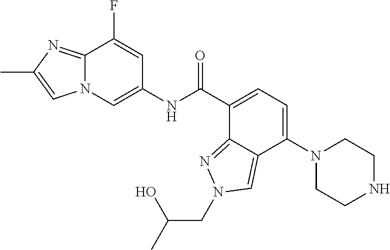 [SMILES: Cc1cn2cc(NC(=O)c3ccc(N4CCNCC4)c4cn(CC(C)O)nc34)cc(F)c2n1] |
| 358 | 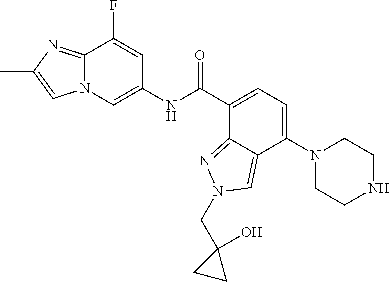 [SMILES: Cc1cn2cc(NC(=O)c3ccc(N4CCNCC4)c4cn(CC5(O)CC5)nc34)cc(F)c2n1] |
| 359 | 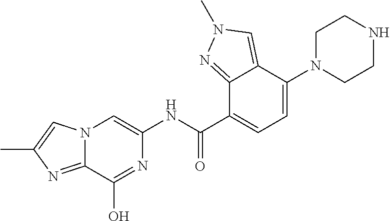 [SMILES: Cc1cn2cc(NC(=O)c3ccc(N4CCNCC4)c4cn(C)nc34)nc(O)c2n1] |
| 360 | 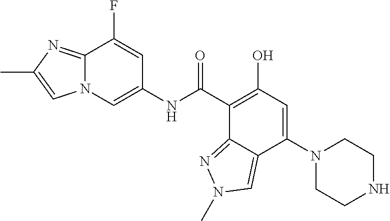 [SMILES: Cc1cn2cc(NC(=O)c3c(O)cc(N4CCNCC4)c4cn(C)nc34)cc(F)c2n1] |
| 361 | 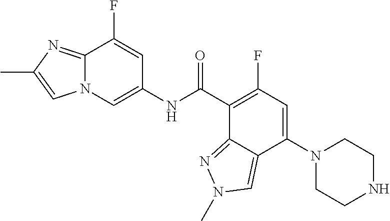 [SMILES: Cc1cn2cc(NC(=O)c3c(F)cc(N4CCNCC4)c4cn(C)nc34)cc(F)c2n1] |
| 362 | 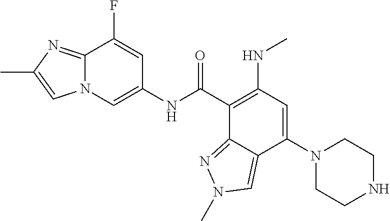 [SMILES: CNc1cc(N2CCNCC2)c2cn(C)nc2c1C(=O)Nc1cc(F)c2nc(C)cn2c1] |
| 363 | 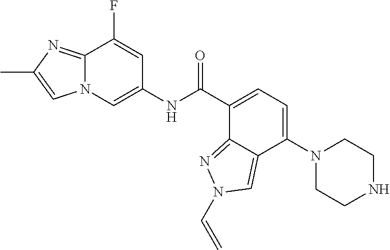 [SMILES: C=Cn1cc2c(N3CCNCC3)ccc(C(=O)Nc3cc(F)c4nc(C)cn4c3)c2n1] |
| 364 | 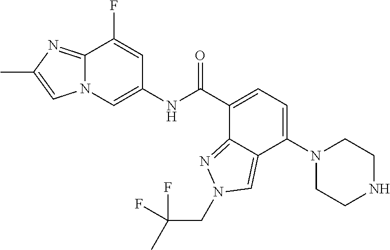 [SMILES: Cc1cn2cc(NC(=O)c3ccc(N4CCNCC4)c4cn(CC(C)(F)F)nc34)cc(F)c2n1] |
| 365 | 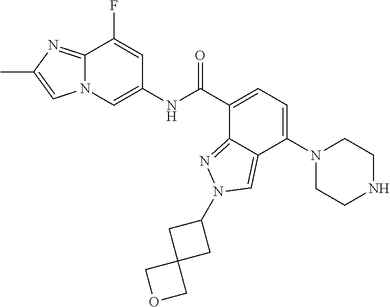 [SMILES: Cc1cn2cc(NC(=O)c3ccc(N4CCNCC4)c4cn(C5CC6(COC6)C5)nc34)cc(F)c2n1] |
| 366 | 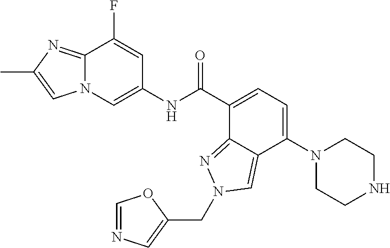 [SMILES: Cc1cn2cc(NC(=O)c3ccc(N4CCNCC4)c4cn(Cc5cnco5)nc34)cc(F)c2n1] |
| 367 | 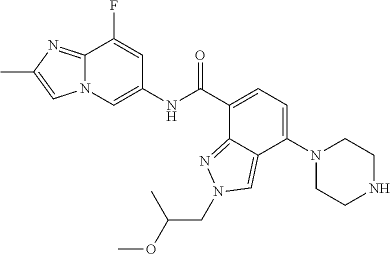 [SMILES: COC(C)Cn1cc2c(N3CCNCC3)ccc(C(=O)Nc3cc(F)c4nc(C)cn4c3)c2n1] |
| 368 | 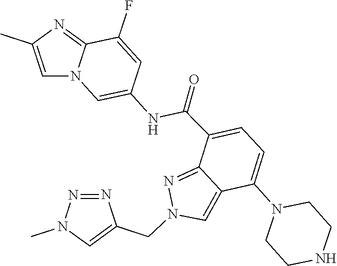 [SMILES: Cc1cn2cc(NC(=O)c3ccc(N4CCNCC4)c4cn(Cc5cn(C)nn5)nc34)cc(F)c2n1] |
| 369 | 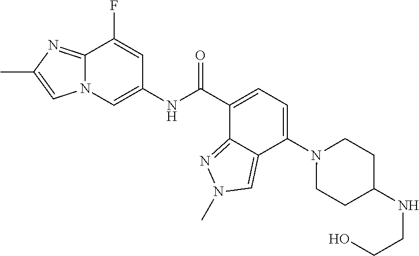 [SMILES: Cc1cn2cc(NC(=O)c3ccc(N4CCC(NCCO)CC4)c4cn(C)nc34)cc(F)c2n1] |
| 370 | 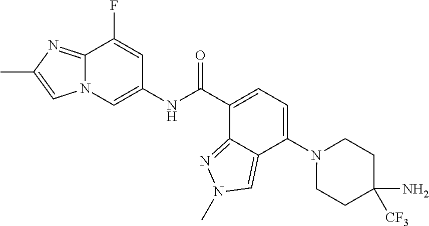 [SMILES: Cc1cn2cc(NC(=O)c3ccc(N4CCC(N)(C(F)(F)F)CC4)c4cn(C)nc34)cc(F)c2n1] |
| 371 | 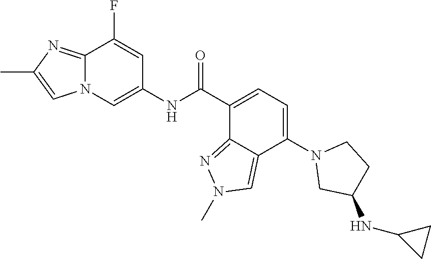 [SMILES: Cc1cn2cc(NC(=O)c3ccc(N4CC[C@@H](NC5CC5)C4)c4cn(C)nc34)cc(F)c2n1] |
| 372 | 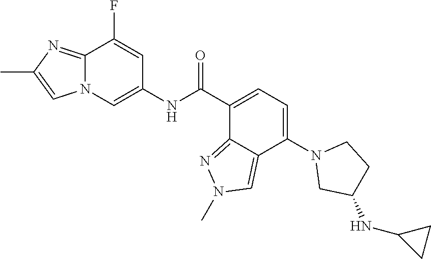 [SMILES: Cc1cn2cc(NC(=O)c3ccc(N4CC[C@H](NC5CC5)C4)c4cn(C)nc34)cc(F)c2n1] |
| 373 | 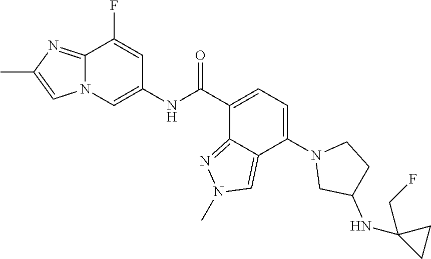 [SMILES: Cc1cn2cc(NC(=O)c3ccc(N4CCC(NC5(CF)CC5)C4)c4cn(C)nc34)cc(F)c2n1] |
| 374 | 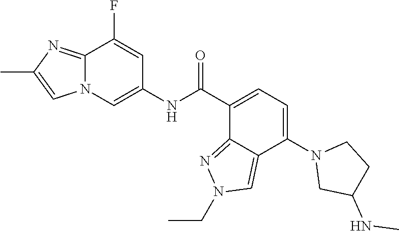 |
| 375 | 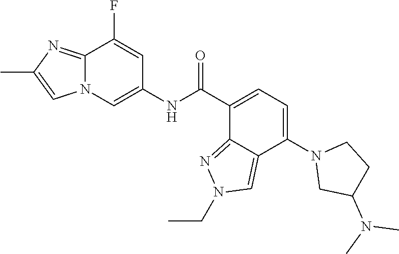 |
| 376 | 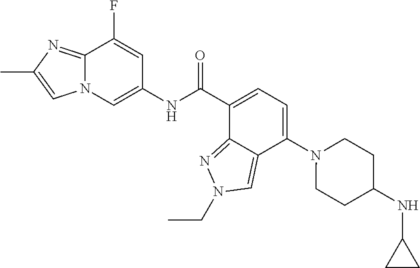 [SMILES: CCn1cc2c(N3CCC(NC4CC4)CC3)ccc(C(=O)Nc3cc(F)c4nc(C)cn4c3)c2n1] |
| 377 | 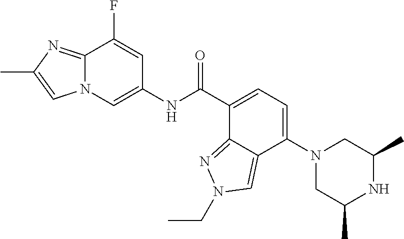 |
| 378 | 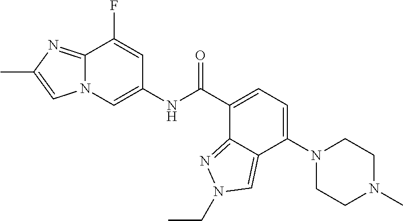 |
| 379 | 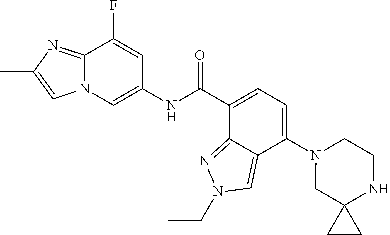 [SMILES: CCn1cc2c(N3CCNC4(CC4)C3)ccc(C(=O)Nc3cc(F)c4nc(C)cn4c3)c2n1] |
| 380 | 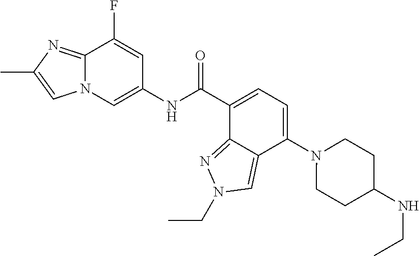 [SMILES: CCNC1CCN(c2ccc(C(=O)Nc3cc(F)c4nc(C)cn4c3)c3nn(CC)cc23)CC1] |
| 381 | 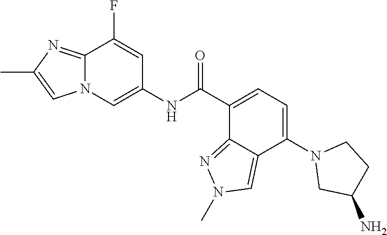 [SMILES: Cc1cn2cc(NC(=O)c3ccc(N4CC[C@@H](N)C4)c4cn(C)nc34)cc(F)c2n1] |
| 382 | 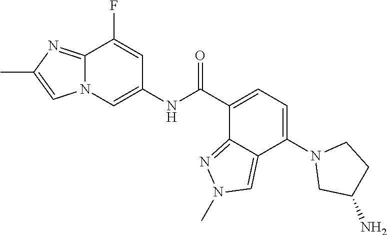 [SMILES: Cc1cn2cc(NC(=O)c3ccc(N4CC[C@H](N)C4)c4cn(C)nc34)cc(F)c2n1] |
| 383 | 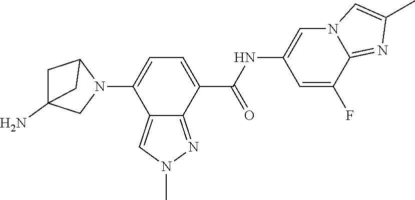 [SMILES: Cc1cn2cc(NC(=O)c3ccc(N4CC5(N)CC4C5)c4cn(C)nc34)cc(F)c2n1] |
| 384 | 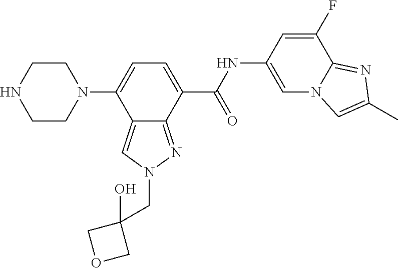 [SMILES: Cc1cn2cc(NC(=O)c3ccc(N4CCNCC4)c4cn(CC5(O)COC5)nc34)cc(F)c2n1] |
| 385 | 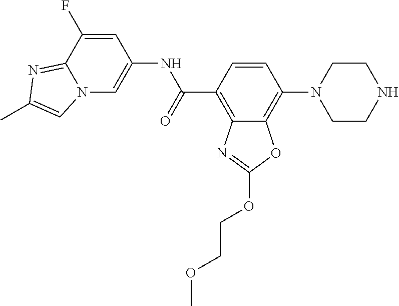 [SMILES: COCCOc1nc2c(C(=O)Nc3cc(F)c4nc(C)cn4c3)ccc(N3CCNCC3)c2o1] |
| 386 | 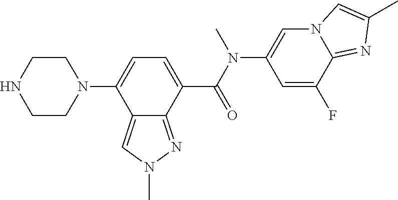 [SMILES: Cc1cn2cc(N(C)C(=O)c3ccc(N4CCNCC4)c4cn(C)nc34)cc(F)c2n1] |
| 387 | 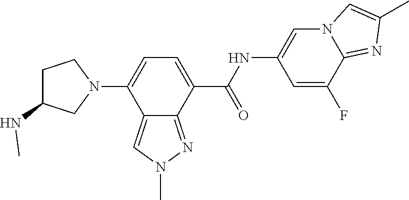 [SMILES: CN[C@H]1CCN(c2ccc(C(=O)Nc3cc(F)c4nc(C)cn4c3)c3nn(C)cc23)C1] |
| 388 | 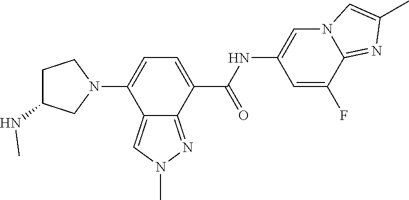 [SMILES: CN[C@@H]1CCN(c2ccc(C(=O)Nc3cc(F)c4nc(C)cn4c3)c3nn(C)cc23)C1] |
| 389 | 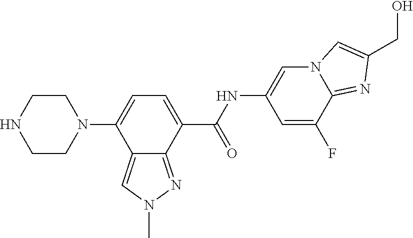 [SMILES: Cn1cc2c(N3CCNCC3)ccc(C(=O)Nc3cc(F)c4nc(CO)cn4c3)c2n1] |
| 390 | 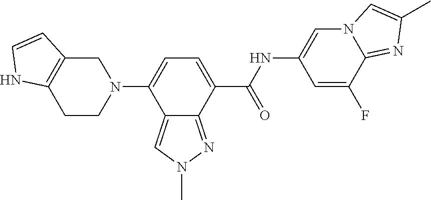 [SMILES: Cc1cn2cc(NC(=O)c3ccc(N4CCc5[nH]ccc5C4)c4cn(C)nc34)cc(F)c2n1] |
| 391 | 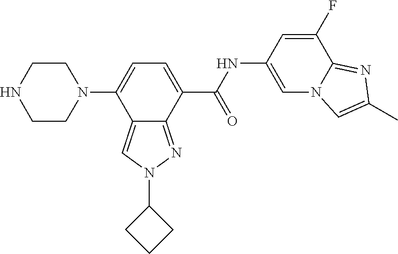 [SMILES: Cc1cn2cc(NC(=O)c3ccc(N4CCNCC4)c4cn(C5CCC5)nc34)cc(F)c2n1] |
| 392 | 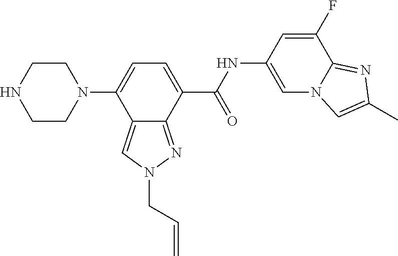 [SMILES: C=CCn1cc2c(N3CCNCC3)ccc(C(=O)Nc3cc(F)c4nc(C)cn4c3)c2n1] |
| 393 | 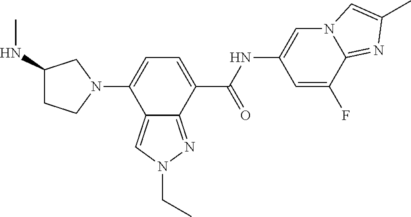 [SMILES: CCn1cc2c(N3CC[C@@H](NC)C3)ccc(C(=O)Nc3cc(F)c4nc(C)cn4c3)c2n1] |
| 394 | 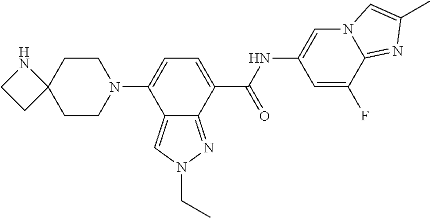 [SMILES: CCn1cc2c(N3CCC4(CCN4)CC3)ccc(C(=O)Nc3cc(F)c4nc(C)cn4c3)c2n1] |
| 395 | 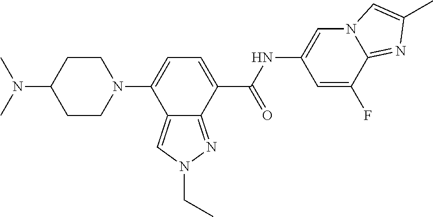 |
| 396 | 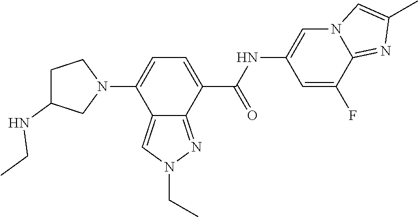 [SMILES: CCNC1CCN(c2ccc(C(=O)Nc3cc(F)c4nc(C)cn4c3)c3nn(CC)cc23)C1] |
| 397 | 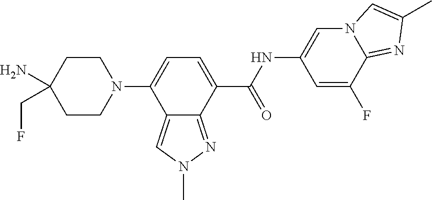 [SMILES: Cc1cn2cc(NC(=O)c3ccc(N4CCC(N)(CF)CC4)c4cn(C)nc34)cc(F)c2n1] |
| 398 | 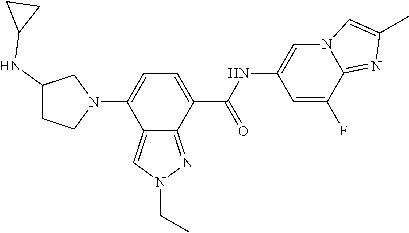 [SMILES: CCn1cc2c(N3CCC(NC4CC4)C3)ccc(C(=O)Nc3cc(F)c4nc(C)cn4c3)c2n1] |
| 399 | 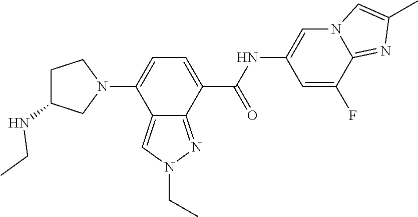 [SMILES: CCN[C@@H]1CCN(c2ccc(C(=O)Nc3cc(F)c4nc(C)cn4c3)c3nn(CC)cc23)C1] |
| 400 | 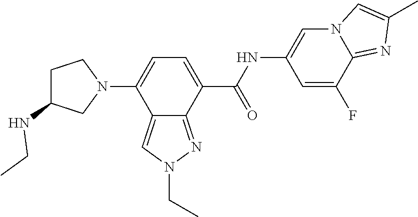 [SMILES: CCN[C@H]1CCN(c2ccc(C(=O)Nc3cc(F)c4nc(C)cn4c3)c3nn(CC)cc23)C1] |
| 401 | 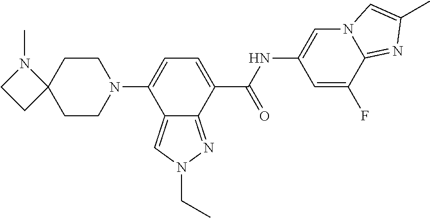 [SMILES: CCn1cc2c(N3CCC4(CC3)CCN4C)ccc(C(=O)Nc3cc(F)c4nc(C)cn4c3)c2n1] |
| 402 | 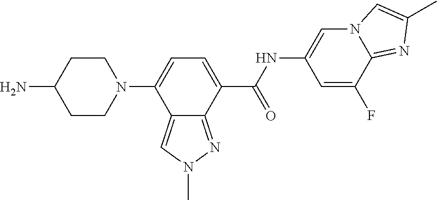 [SMILES: Cc1cn2cc(NC(=O)c3ccc(N4CCC(N)CC4)c4cn(C)nc34)cc(F)c2n1] |
| 403 | 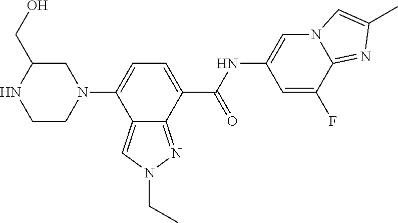 [SMILES: CCn1cc2c(N3CCNC(CO)C3)ccc(C(=O)Nc3cc(F)c4nc(C)cn4c3)c2n1] |
| 404 | 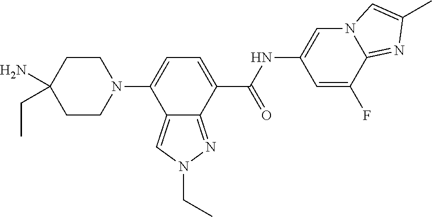 [SMILES: CCn1cc2c(N3CCC(N)(CC)CC3)ccc(C(=O)Nc3cc(F)c4nc(C)cn4c3)c2n1] |
| 405 | 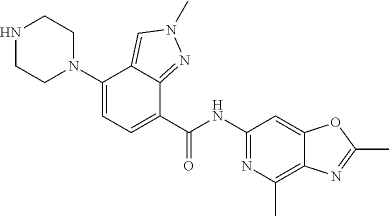 [SMILES: Cc1nc2c(C)nc(NC(=O)c3ccc(N4CCNCC4)c4cn(C)nc34)cc2o1] |
| 406 | 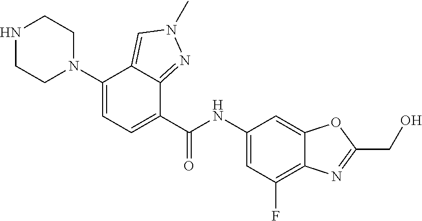 [SMILES: Cn1cc2c(N3CCNCC3)ccc(C(=O)Nc3cc(F)c4nc(CO)oc4c3)c2n1] |
| 407 | 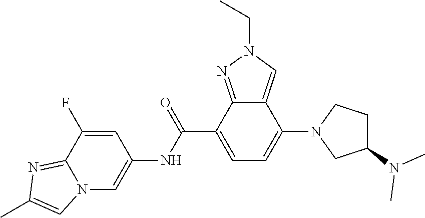 |
| 408 | 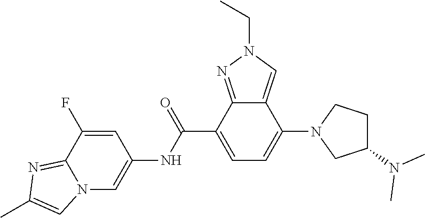 |
| 409 | 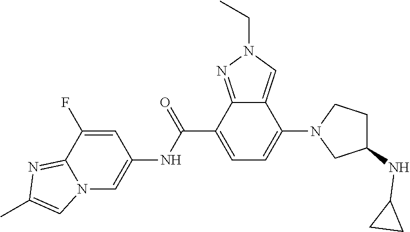 [SMILES: CCn1cc2c(N3CC[C@@H](NC4CC4)C3)ccc(C(=O)Nc3cc(F)c4nc(C)cn4c3)c2n1] |
| 410 | 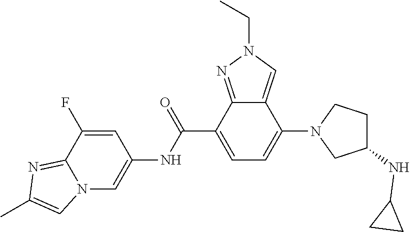 [SMILES: CCn1cc2c(N3CC[C@H](NC4CC4)C3)ccc(C(=O)Nc3cc(F)c4nc(C)cn4c3)c2n1] |
| 411 | 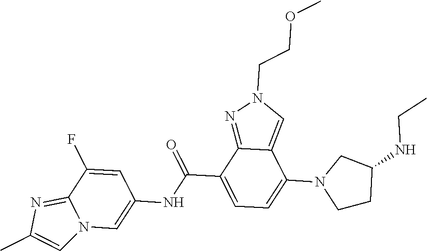 [SMILES: CCN[C@@H]1CCN(c2ccc(C(=O)Nc3cc(F)c4nc(C)cn4c3)c3nn(CCOC)cc23)C1] |
| 412 | 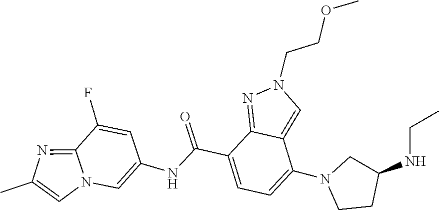 [SMILES: CCN[C@H]1CCN(c2ccc(C(=O)Nc3cc(F)c4nc(C)cn4c3)c3nn(CCOC)cc23)C1] |
| 413 | 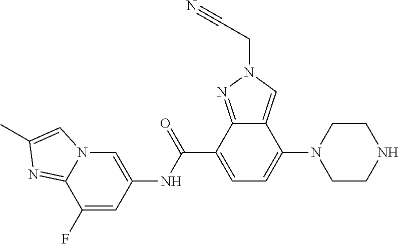 [SMILES: Cc1cn2cc(NC(=O)c3ccc(N4CCNCC4)c4cn(CC#N)nc34)cc(F)c2n1] |
| 414 | 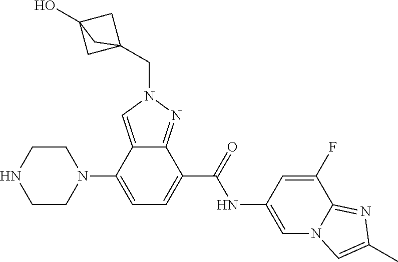 [SMILES: Cc1cn2cc(NC(=O)c3ccc(N4CCNCC4)c4cn(CC56CC(O)(C5)C6)nc34)cc(F)c2n1] |
| 415 | 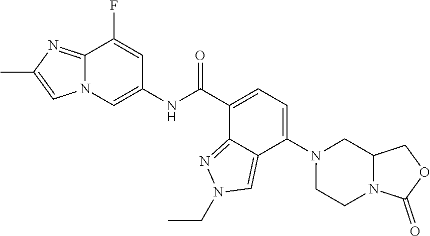 [SMILES: CCn1cc2c(N3CCN4C(=O)OCC4C3)ccc(C(=O)Nc3cc(F)c4nc(C)cn4c3)c2n1] |
| 416 | 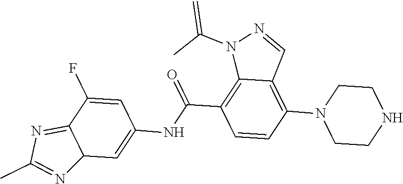 [SMILES: C=C(C)n1ncc2c(N3CCNCC3)ccc(C(=O)NC3=CC4N=C(C)N=C4C(F)=C3)c21] |
| 417 | 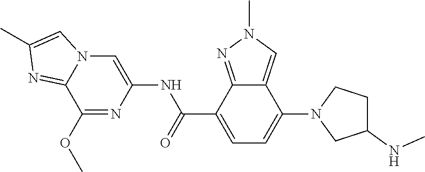 |
| 418 | 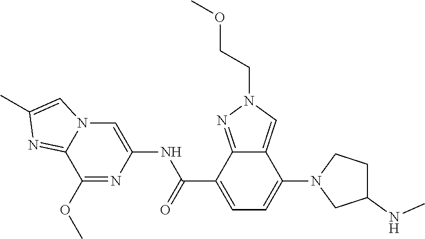 |
| 419 | 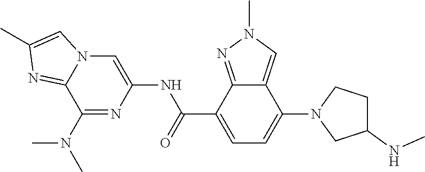 |
| 420 | 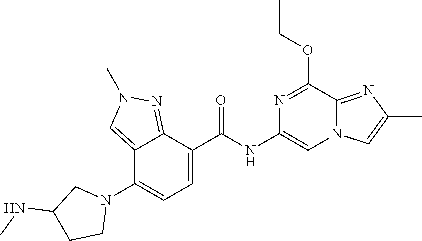 |
| 421 | 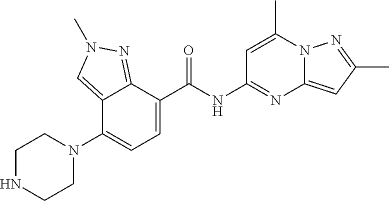 [SMILES: Cc1cc2nc(NC(=O)c3ccc(N4CCNCC4)c4cn(C)nc34)cc(C)n2n1] |
| 422 | 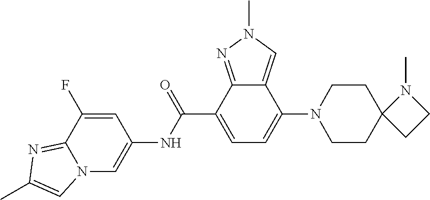 [SMILES: Cc1cn2cc(NC(=O)c3ccc(N4CCC5(CC4)CCN5C)c4cn(C)nc34)cc(F)c2n1] |
| 423 | 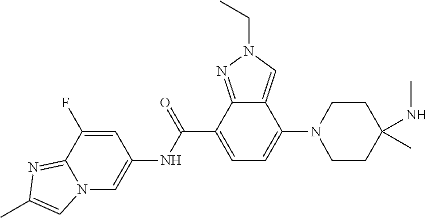 |
| 424 | 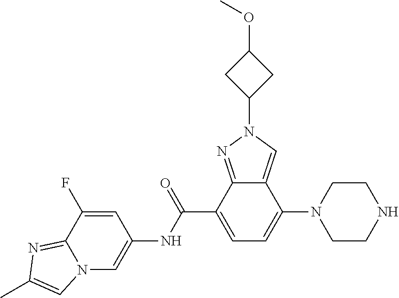 [SMILES: COC1CC(n2cc3c(N4CCNCC4)ccc(C(=O)Nc4cc(F)c5nc(C)cn5c4)c3n2)C1] |
| 425 | 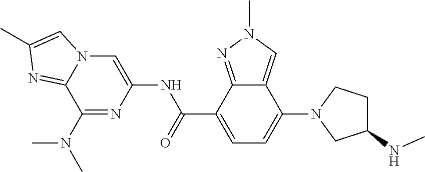 |
| 426 | 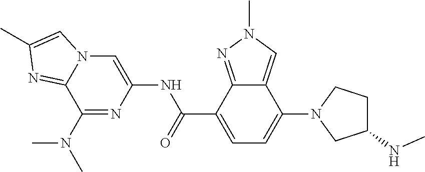 |
| 427 | 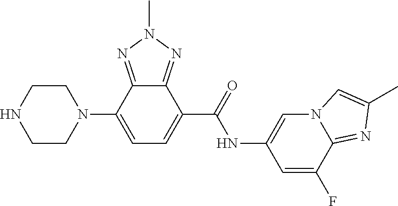 [SMILES: Cc1cn2cc(NC(=O)c3ccc(N4CCNCC4)c4nn(C)nc34)cc(F)c2n1] |
| 428 | 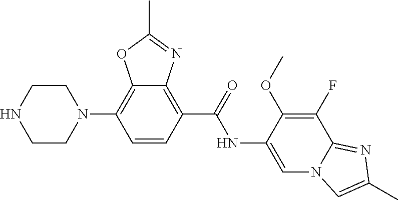 [SMILES: COc1c(NC(=O)c2ccc(N3CCNCC3)c3oc(C)nc23)cn2cc(C)nc2c1F] |
| 429 | 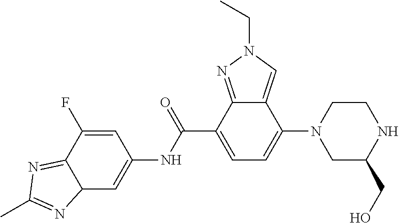 [SMILES: CCn1cc2c(N3CCN[C@@H](CO)C3)ccc(C(=O)NC3=CC4N=C(C)N=C4C(F)=C3)c2n1] |
| 430 | 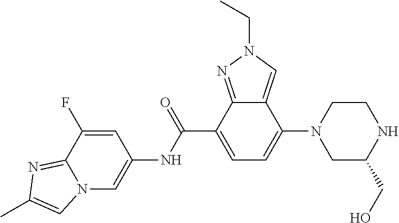 [SMILES: CCn1cc2c(N3CCN[C@H](CO)C3)ccc(C(=O)Nc3cc(F)c4nc(C)cn4c3)c2n1] |
| 431 | 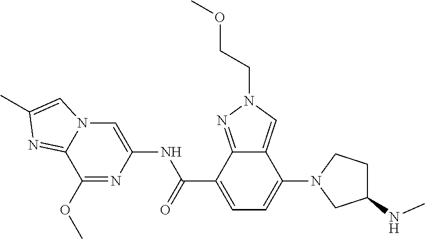 |
| 432 | 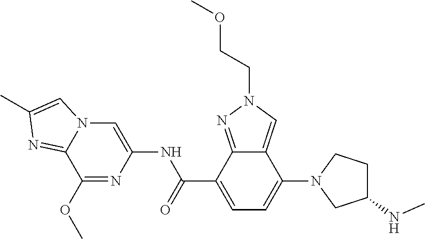 |
| 433 | 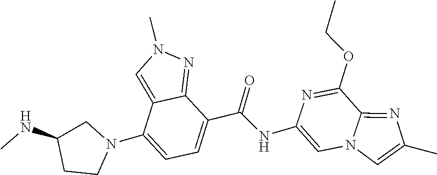 |
| 434 | 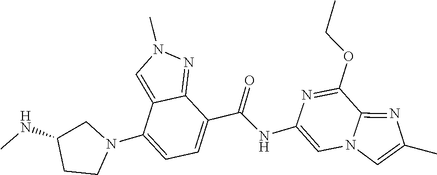 |
| 435 | 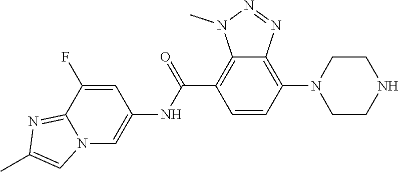 [SMILES: Cc1cn2cc(NC(=O)c3ccc(N4CCNCC4)c4nnn(C)c34)cc(F)c2n1] |
| 436 | 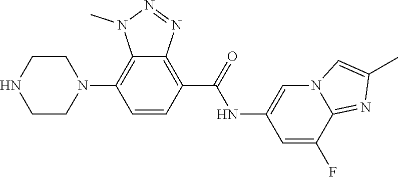 [SMILES: Cc1cn2cc(NC(=O)c3ccc(N4CCNCC4)c4c3nnn4C)cc(F)c2n1] |
| 437 | 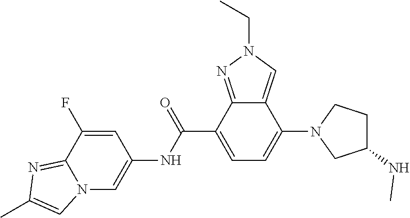 [SMILES: CCn1cc2c(N3CC[C@H](NC)C3)ccc(C(=O)Nc3cc(F)c4nc(C)cn4c3)c2n1] |
| 438 | 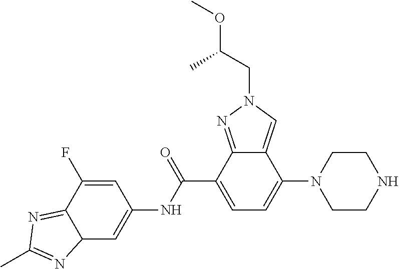 [SMILES: CO[C@@H](C)Cn1cc2c(N3CCNCC3)ccc(C(=O)NC3=CC4N=C(C)N=C4C(F)=C3)c2n1] |
| 439 | 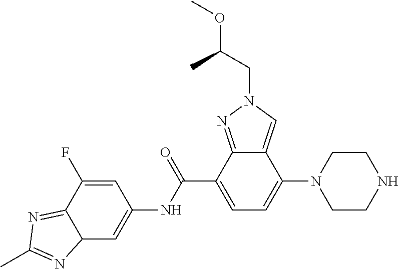 [SMILES: CO[C@H](C)Cn1cc2c(N3CCNCC3)ccc(C(=O)NC3=CC4N=C(C)N=C4C(F)=C3)c2n1] |
| 440 | 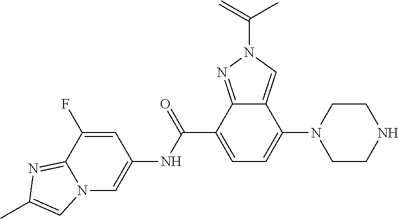 [SMILES: C=C(C)n1cc2c(N3CCNCC3)ccc(C(=O)Nc3cc(F)c4nc(C)cn4c3)c2n1] |
| 441 | 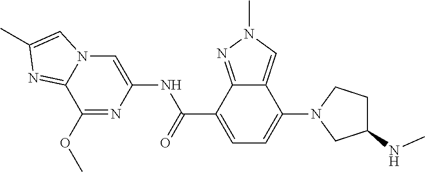 |
| 442 | 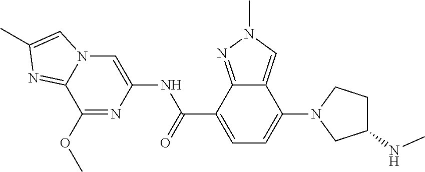 |
| 443 | 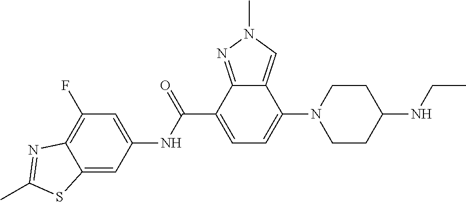 [SMILES: CCNC1CCN(c2ccc(C(=O)Nc3cc(F)c4nc(C)sc4c3)c3nn(C)cc23)CC1] |
| 444 | 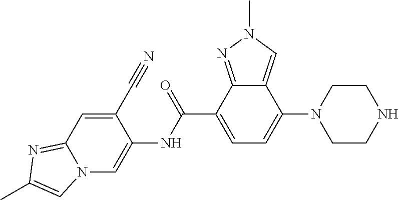 [SMILES: Cc1cn2cc(NC(=O)c3ccc(N4CCNCC4)c4cn(C)nc34)c(C#N)cc2n1] |
| 445 | 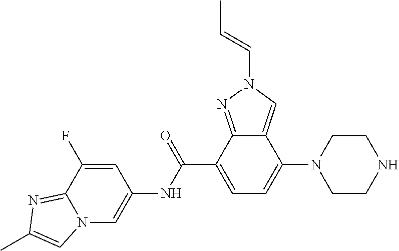 [SMILES: C/C=C/n1cc2c(N3CCNCC3)ccc(C(=O)Nc3cc(F)c4nc(C)cn4c3)c2n1] |
| 446 | 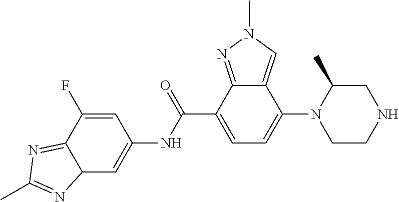 [SMILES: CC1=NC2C=C(NC(=O)c3ccc(N4CCNC[C@@H]4C)c4cn(C)nc34)C=C(F)C2=N1] |
| 447 | 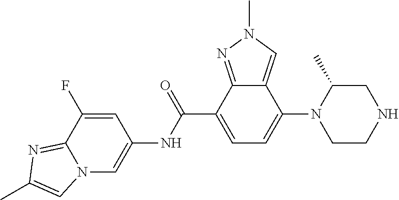 [SMILES: Cc1cn2cc(NC(=O)c3ccc(N4CCNC[C@H]4C)c4cn(C)nc34)cc(F)c2n1] |
| 448 | 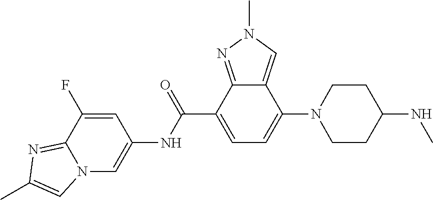 |
| 449 | 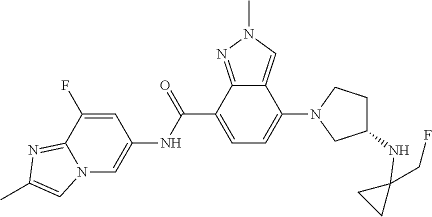 [SMILES: Cc1cn2cc(NC(=O)c3ccc(N4CC[C@H](NC5(CF)CC5)C4)c4cn(C)nc34)cc(F)c2n1] |
| 450 | 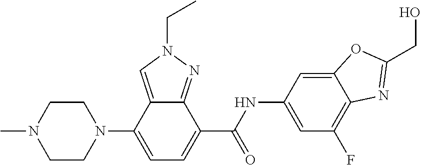 [SMILES: CCn1cc2c(N3CCN(C)CC3)ccc(C(=O)Nc3cc(F)c4nc(CO)oc4c3)c2n1] |
| 451 | 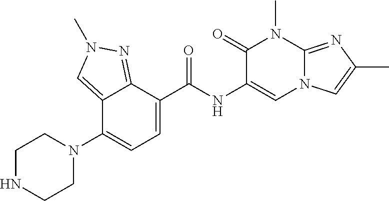 [SMILES: Cc1cn2cc(NC(=O)c3ccc(N4CCNCC4)c4cn(C)nc34)c(=O)n(C)c2n1] |
| 452 | 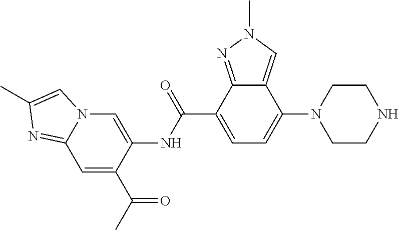 [SMILES: CC(=O)c1cc2nc(C)cn2cc1NC(=O)c1ccc(N2CCNCC2)c2cn(C)nc12] |
| 453 | 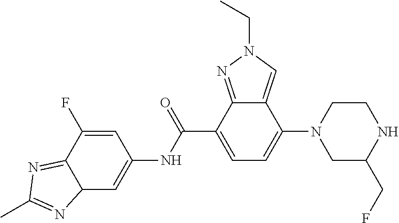 [SMILES: CCn1cc2c(N3CCNC(CF)C3)ccc(C(=O)NC3=CC4N=C(C)N=C4C(F)=C3)c2n1] |
| 454 | 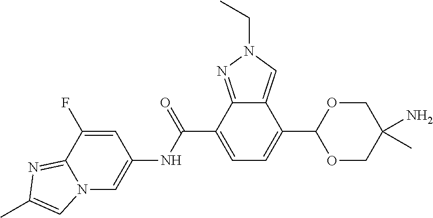 |
| 455 | 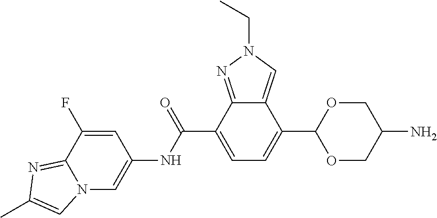 [SMILES: CCn1cc2c(C3OCC(N)CO3)ccc(C(=O)Nc3cc(F)c4nc(C)cn4c3)c2n1] |
| 456 | 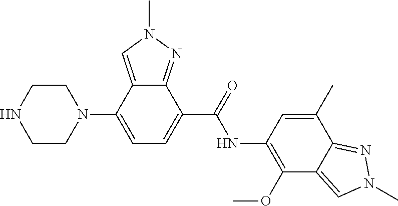 [SMILES: COc1c(NC(=O)c2ccc(N3CCNCC3)c3cn(C)nc23)cc(C)c2nn(C)cc12] |
| 457 | 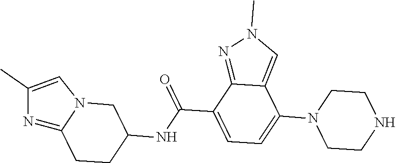 [SMILES: Cc1cn2c(n1)CCC(NC(=O)c1ccc(N3CCNCC3)c3cn(C)nc13)C2] |
| 458 | 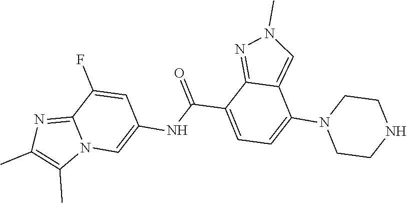 [SMILES: Cc1nc2c(F)cc(NC(=O)c3ccc(N4CCNCC4)c4cn(C)nc34)cn2c1C] |
| 459 | 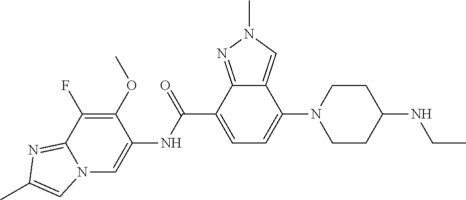 [SMILES: CCNC1CCN(c2ccc(C(=O)Nc3cn4cc(C)nc4c(F)c3OC)c3nn(C)cc23)CC1] |
| 460 | 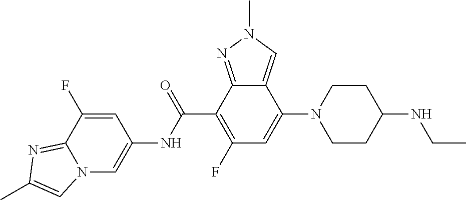 [SMILES: CCNC1CCN(c2cc(F)c(C(=O)Nc3cc(F)c4nc(C)cn4c3)c3nn(C)cc23)CC1] |
| 461 | 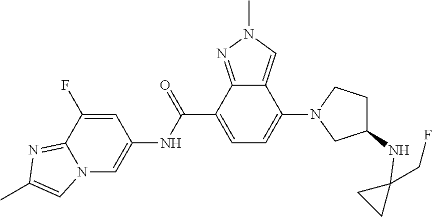 [SMILES: Cc1cn2cc(NC(=O)c3ccc(N4CC[C@@H](NC5(CF)CC5)C4)c4cn(C)nc34)cc(F)c2n1] |
| 462 | 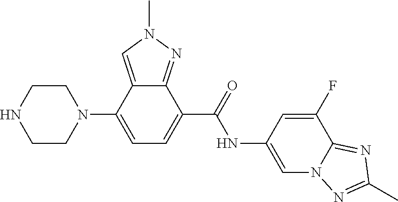 [SMILES: Cc1nc2c(F)cc(NC(=O)c3ccc(N4CCNCC4)c4cn(C)nc34)cn2n1] |
| 463 | 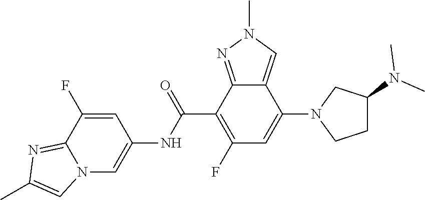 |
| 464 | 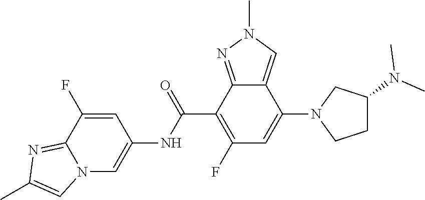 |
| 468 | 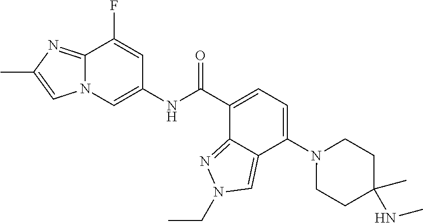 |
| 469 | 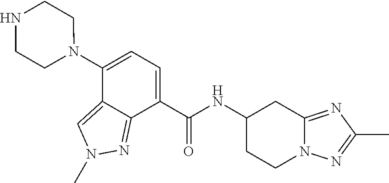 [SMILES: Cc1nc2n(n1)CCC(NC(=O)c1ccc(N3CCNCC3)c3cn(C)nc13)C2] |
| 470 | 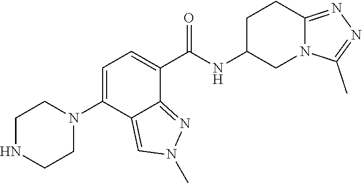 [SMILES: Cc1nnc2n1CC(NC(=O)c1ccc(N3CCNCC3)c3cn(C)nc13)CC2] |
| 471 | 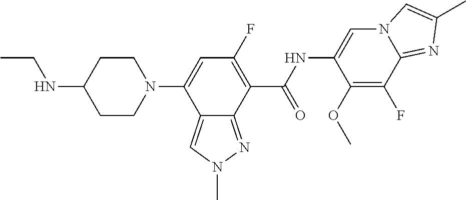 [SMILES: CCNC1CCN(c2cc(F)c(C(=O)Nc3cn4cc(C)nc4c(F)c3OC)c3nn(C)cc23)CC1] |
| 472 | 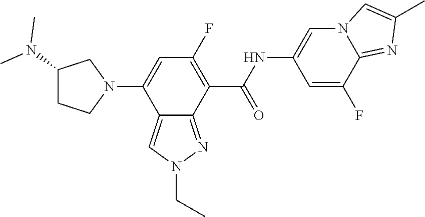 |
| 473 | 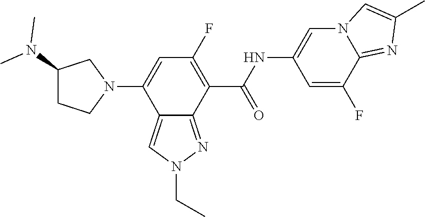 |
| 474 | 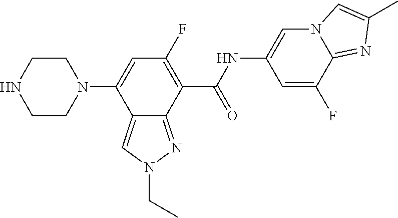 [SMILES: CCn1cc2c(N3CCNCC3)cc(F)c(C(=O)Nc3cc(F)c4nc(C)cn4c3)c2n1] |
| 475 | 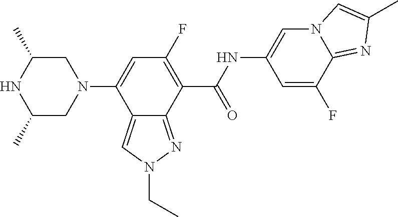 [SMILES: CCn1cc2c(N3C[C@@H](C)N[C@@H](C)C3)cc(F)c(C(=O)Nc3cc(F)c4nc(C)cn4c3)c2n1] |
| 476 | 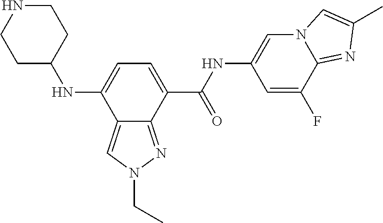 [SMILES: CCn1cc2c(NC3CCNCC3)ccc(C(=O)Nc3cc(F)c4nc(C)cn4c3)c2n1] |
| 477 | 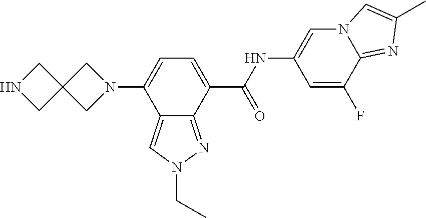 [SMILES: CCn1cc2c(N3CC4(CNC4)C3)ccc(C(=O)Nc3cc(F)c4nc(C)cn4c3)c2n1] |
| 478 | 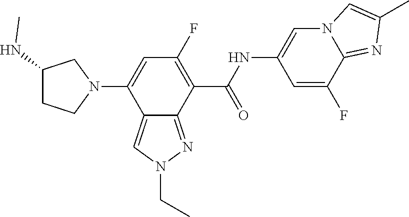 [SMILES: CCn1cc2c(N3CC[C@H](NC)C3)cc(F)c(C(=O)Nc3cc(F)c4nc(C)cn4c3)c2n1] |
| 479 | 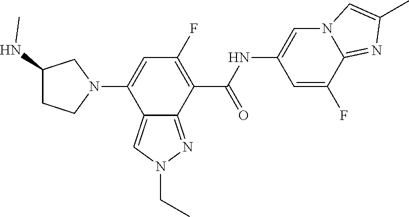 [SMILES: CCn1cc2c(N3CC[C@@H](NC)C3)cc(F)c(C(=O)Nc3cc(F)c4nc(C)cn4c3)c2n1] |
| 480 | 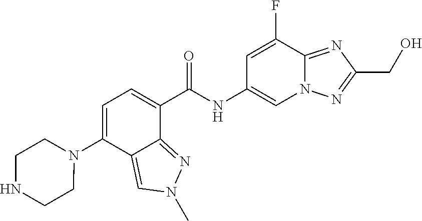 [SMILES: Cn1cc2c(N3CCNCC3)ccc(C(=O)Nc3cc(F)c4nc(CO)nn4c3)c2n1] |
| 481 | 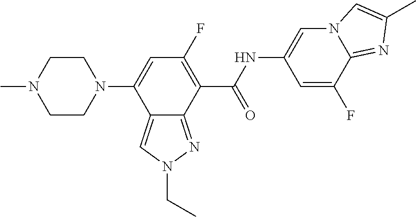 |
| 482 | 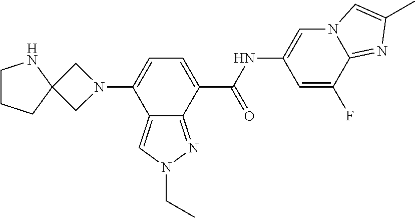 [SMILES: CCn1cc2c(N3CC4(CCCN4)C3)ccc(C(=O)Nc3cc(F)c4nc(C)cn4c3)c2n1] |
| 483 | 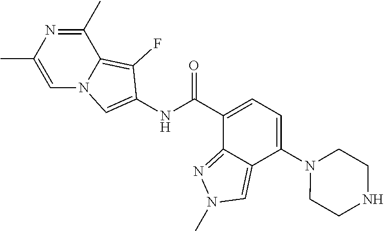 [SMILES: Cc1cn2cc(NC(=O)c3ccc(N4CCNCC4)c4cn(C)nc34)c(F)c2c(C)n1] |
| 484 | 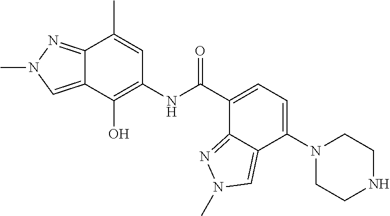 [SMILES: Cc1cc(NC(=O)c2ccc(N3CCNCC3)c3cn(C)nc23)c(O)c2cn(C)nc12] |
| 485 | 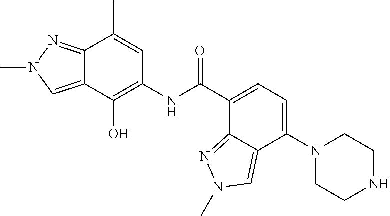 [SMILES: Cc1cc(NC(=O)c2ccc(N3CCNCC3)c3cn(C)nc23)c(O)c2cn(C)nc12] |
| 486 | 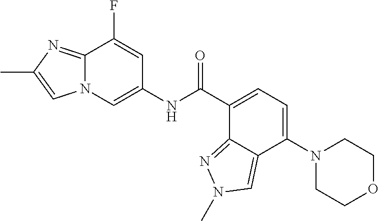 [SMILES: Cc1cn2cc(NC(=O)c3ccc(N4CCOCC4)c4cn(C)nc34)cc(F)c2n1] |
| 487 | 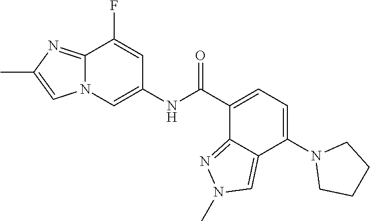 [SMILES: Cc1cn2cc(NC(=O)c3ccc(N4CCCC4)c4cn(C)nc34)cc(F)c2n1] |
| 488 | 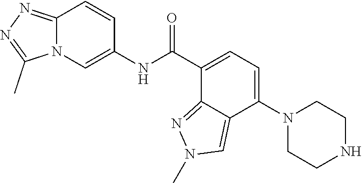 [SMILES: Cc1nnc2ccc(NC(=O)c3ccc(N4CCNCC4)c4cn(C)nc34)cn12] |
| 489 | 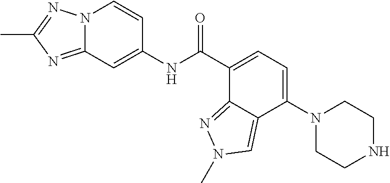 [SMILES: Cc1nc2cc(NC(=O)c3ccc(N4CCNCC4)c4cn(C)nc34)ccn2n1] |
| 490 | 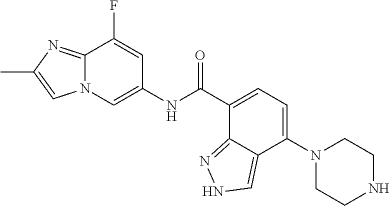 [SMILES: Cc1cn2cc(NC(=O)c3ccc(N4CCNCC4)c4c[nH]nc34)cc(F)c2n1] |
| 491 | 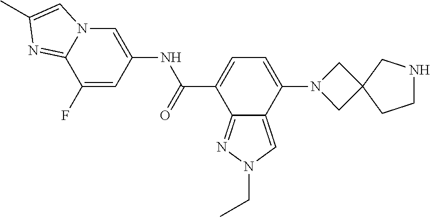 [SMILES: CCn1cc2c(N3CC4(CCNC4)C3)ccc(C(=O)Nc3cc(F)c4nc(C)cn4c3)c2n1] |
| 492 | 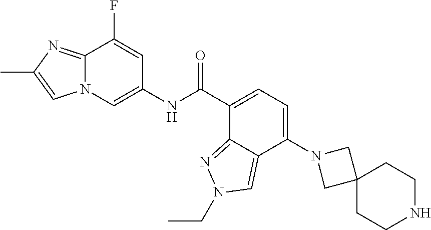 [SMILES: CCn1cc2c(N3CC4(CCNCC4)C3)ccc(C(=O)Nc3cc(F)c4nc(C)cn4c3)c2n1] |
| 493 | 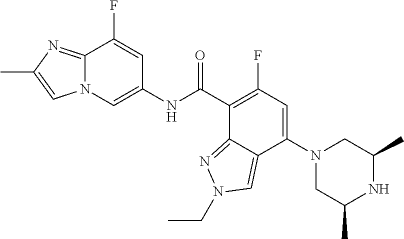 |
| 494 | 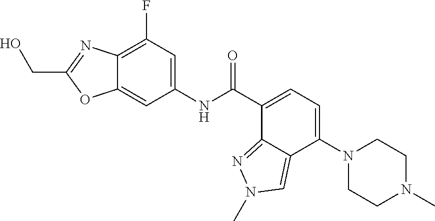 [SMILES: CN1CCN(c2ccc(C(=O)Nc3cc(F)c4nc(CO)oc4c3)c3nn(C)cc23)CC1] |
| 495 | 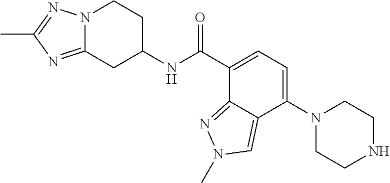 [SMILES: Cc1nc2n(n1)CCC(NC(=O)c1ccc(N3CCNCC3)c3cn(C)nc13)C2] |
| 496 | 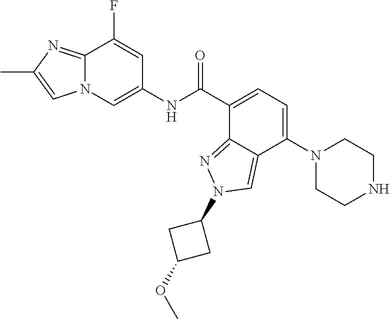 [SMILES: CO[C@H]1C[C@H](n2cc3c(N4CCNCC4)ccc(C(=O)Nc4cc(F)c5nc(C)cn5c4)c3n2)C1] |
| 497 | 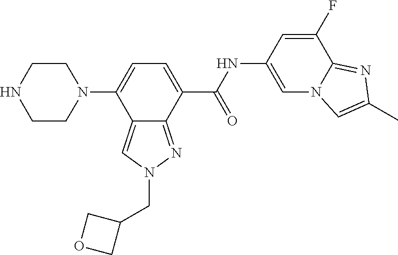 [SMILES: Cc1cn2cc(NC(=O)c3ccc(N4CCNCC4)c4cn(CC5COC5)nc34)cc(F)c2n1] |
| 498 | 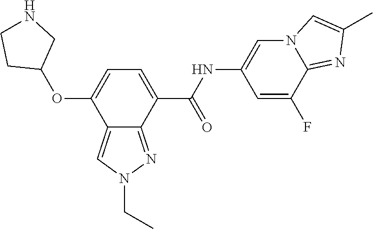 [SMILES: CCn1cc2c(OC3CCNC3)ccc(C(=O)Nc3cc(F)c4nc(C)cn4c3)c2n1] |
| 499 | 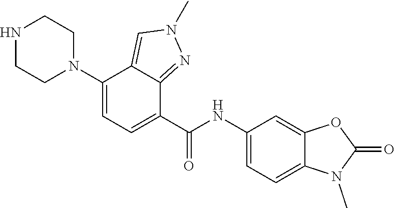 [SMILES: Cn1cc2c(N3CCNCC3)ccc(C(=O)Nc3ccc4c(c3)oc(=O)n4C)c2n1] |
| 500 | 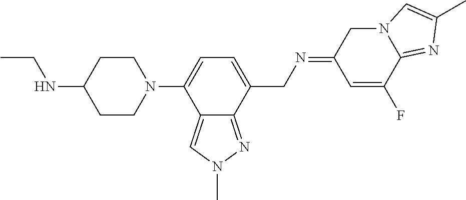 [SMILES: CCNC1CCN(c2ccc(C/N=C3\C=C(F)c4nc(C)cn4C3)c3nn(C)cc23)CC1] |
| 501 | 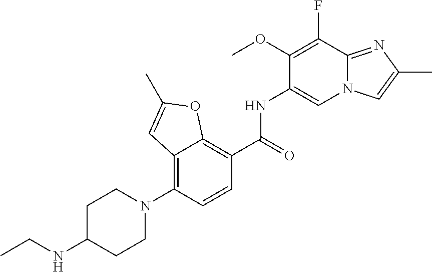 [SMILES: CCNC1CCN(c2ccc(C(=O)Nc3cn4cc(C)nc4c(F)c3OC)c3oc(C)cc23)CC1] |
| 502 | 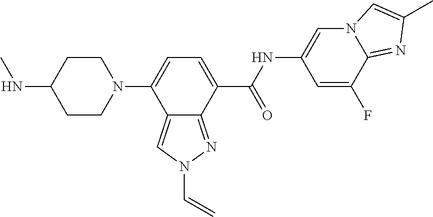 |
| 503 | 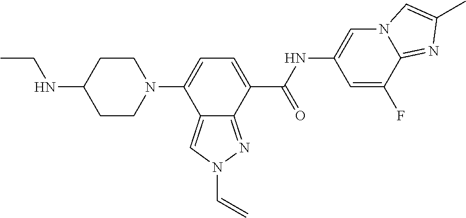 [SMILES: C=Cn1cc2c(N3CCC(NCC)CC3)ccc(C(=O)Nc3cc(F)c4nc(C)cn4c3)c2n1] |
| 504 | 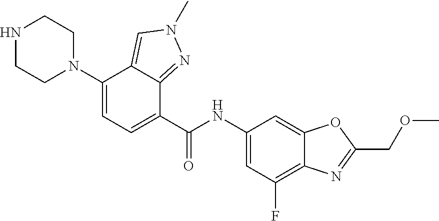 [SMILES: COCc1nc2c(F)cc(NC(=O)c3ccc(N4CCNCC4)c4cn(C)nc34)cc2o1] |
| 505 | 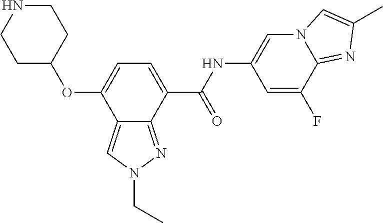 [SMILES: CCn1cc2c(OC3CCNCC3)ccc(C(=O)Nc3cc(F)c4nc(C)cn4c3)c2n1] |
| 506 | 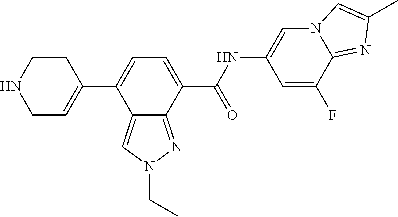 [SMILES: CCn1cc2c(C3=CCNCC3)ccc(C(=O)Nc3cc(F)c4nc(C)cn4c3)c2n1] |
| 507 | 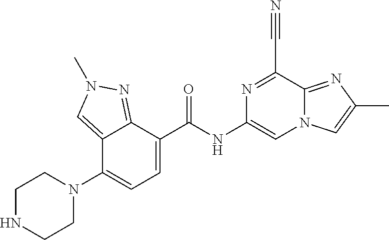 [SMILES: Cc1cn2cc(NC(=O)c3ccc(N4CCNCC4)c4cn(C)nc34)nc(C#N)c2n1] |
| 508 | 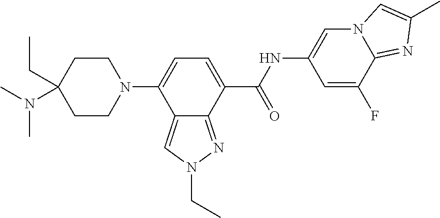 |
| 509 | 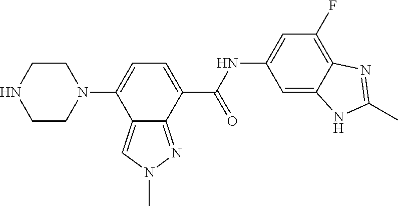 [SMILES: Cc1nc2c(F)cc(NC(=O)c3ccc(N4CCNCC4)c4cn(C)nc34)cc2[nH]1] |
| 510 | 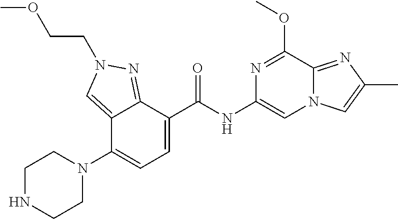 |
| 511 | 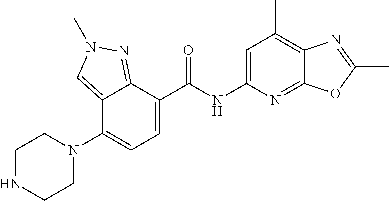 [SMILES: Cc1nc2c(C)cc(NC(=O)c3ccc(N4CCNCC4)c4cn(C)nc34)nc2o1] |
| 512 | 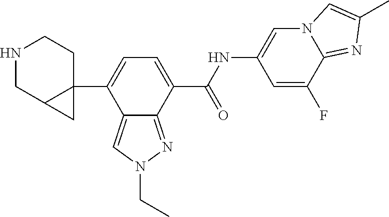 [SMILES: CCn1cc2c(C34CCNCC3C4)ccc(C(=O)Nc3cc(F)c4nc(C)cn4c3)c2n1] |
| 513 | 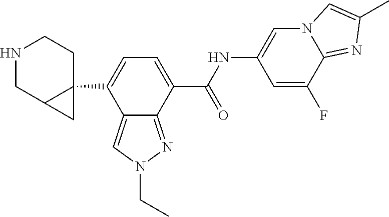 [SMILES: CCn1cc2c([C@@]34CCNCC3C4)ccc(C(=O)Nc3cc(F)c4nc(C)cn4c3)c2n1] |
| 514 | 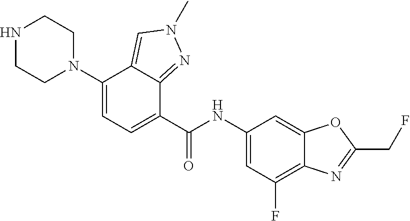 [SMILES: Cn1cc2c(N3CCNCC3)ccc(C(=O)Nc3cc(F)c4nc(CF)oc4c3)c2n1] |
| 515 | 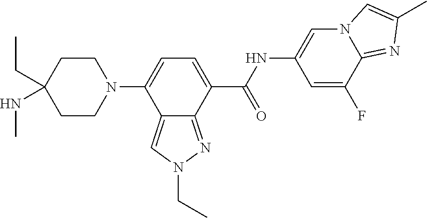 [SMILES: CCn1cc2c(N3CCC(CC)(NC)CC3)ccc(C(=O)Nc3cc(F)c4nc(C)cn4c3)c2n1] |
| 516 | 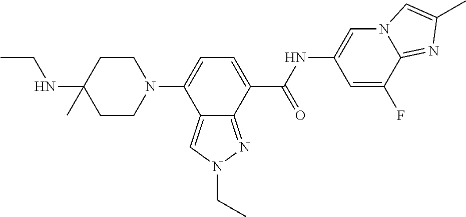 [SMILES: CCNC1(C)CCN(c2ccc(C(=O)Nc3cc(F)c4nc(C)cn4c3)c3nn(CC)cc23)CC1] |
| 517 | 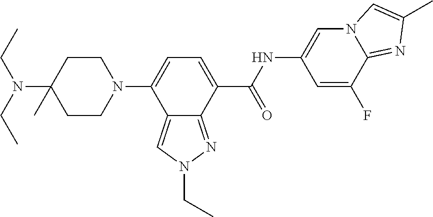 [SMILES: CCN(CC)C1(C)CCN(c2ccc(C(=O)Nc3cc(F)c4nc(C)cn4c3)c3nn(CC)cc23)CC1] |
| 518 | 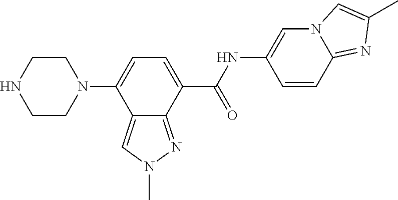 [SMILES: Cc1cn2cc(NC(=O)c3ccc(N4CCNCC4)c4cn(C)nc34)ccc2n1] |
| 519 | 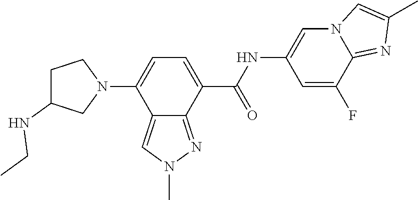 [SMILES: CCNC1CCN(c2ccc(C(=O)Nc3cc(F)c4nc(C)cn4c3)c3nn(C)cc23)C1] |
| 520 | 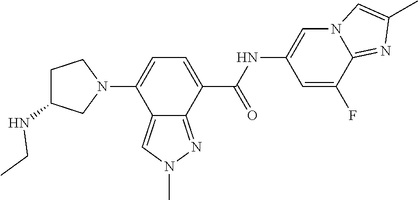 [SMILES: CCN[C@@H]1CCN(c2ccc(C(=O)Nc3cc(F)c4nc(C)cn4c3)c3nn(C)cc23)C1] |
| 521 | 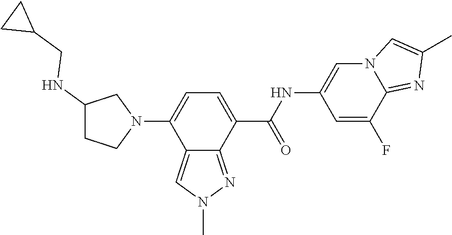 [SMILES: Cc1cn2cc(NC(=O)c3ccc(N4CCC(NCC5CC5)C4)c4cn(C)nc34)cc(F)c2n1] |
| 522 | 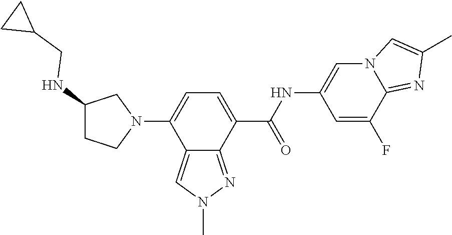 [SMILES: Cc1cn2cc(NC(=O)c3ccc(N4CC[C@@H](NCC5CC5)C4)c4cn(C)nc34)cc(F)c2n1] |
| 523 | 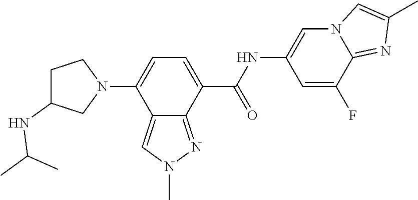 [SMILES: Cc1cn2cc(NC(=O)c3ccc(N4CCC(NC(C)C)C4)c4cn(C)nc34)cc(F)c2n1] |
| 524 | 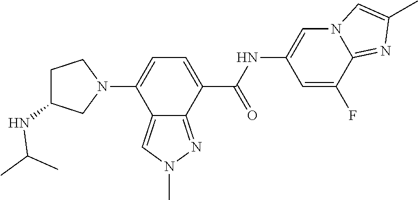 [SMILES: Cc1cn2cc(NC(=O)c3ccc(N4CC[C@@H](NC(C)C)C4)c4cn(C)nc34)cc(F)c2n1] |
| 525 | 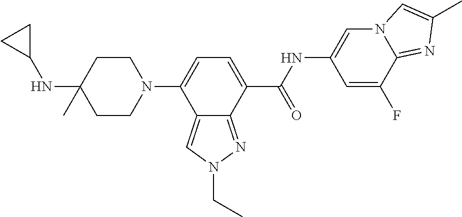 [SMILES: CCn1cc2c(N3CCC(C)(NC4CC4)CC3)ccc(C(=O)Nc3cc(F)c4nc(C)cn4c3)c2n1] |
| 526 | 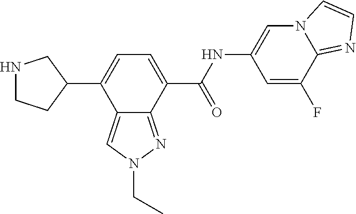 [SMILES: CCn1cc2c(C3CCNC3)ccc(C(=O)Nc3cc(F)c4nccn4c3)c2n1] |
| 527 | 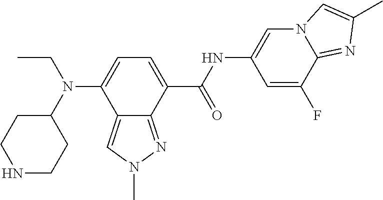 [SMILES: CCN(c1ccc(C(=O)Nc2cc(F)c3nc(C)cn3c2)c2nn(C)cc12)C1CCNCC1] |
| 528 | 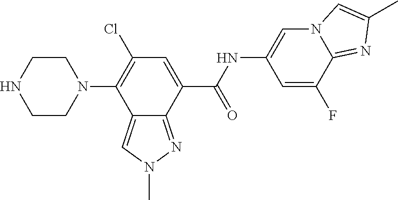 [SMILES: Cc1cn2cc(NC(=O)c3cc(Cl)c(N4CCNCC4)c4cn(C)nc34)cc(F)c2n1] |
| 529 | 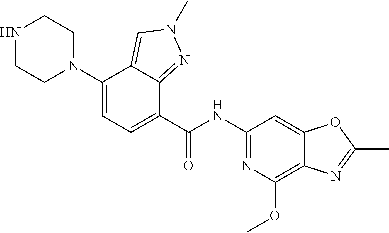 [SMILES: COc1nc(NC(=O)c2ccc(N3CCNCC3)c3cn(C)nc23)cc2oc(C)nc12] |
| 530 | 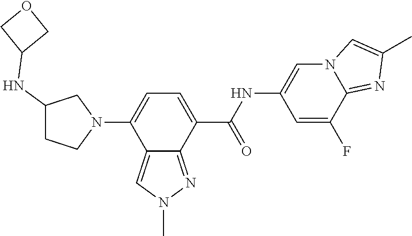 [SMILES: Cc1cn2cc(NC(=O)c3ccc(N4CCC(NC5COC5)C4)c4cn(C)nc34)cc(F)c2n1] |
| 531 | 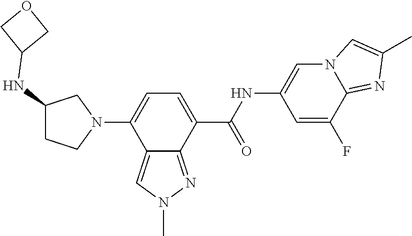 [SMILES: Cc1cn2cc(NC(=O)c3ccc(N4CC[C@@H](NC5COC5)C4)c4cn(C)nc34)cc(F)c2n1] |
| 532 | 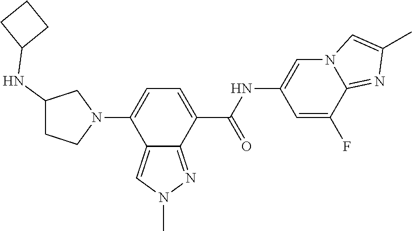 [SMILES: Cc1cn2cc(NC(=O)c3ccc(N4CCC(NC5CCC5)C4)c4cn(C)nc34)cc(F)c2n1] |
| 533 | 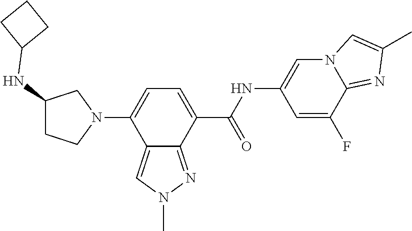 [SMILES: Cc1cn2cc(NC(=O)c3ccc(N4CC[C@@H](NC5CCC5)C4)c4cn(C)nc34)cc(F)c2n1] |
| 534 | 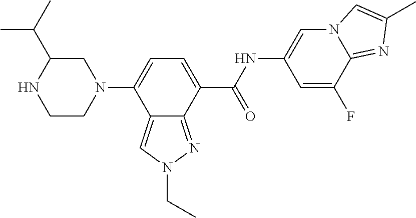 [SMILES: CCn1cc2c(N3CCNC(C(C)C)C3)ccc(C(=O)Nc3cc(F)c4nc(C)cn4c3)c2n1] |
| 535 | 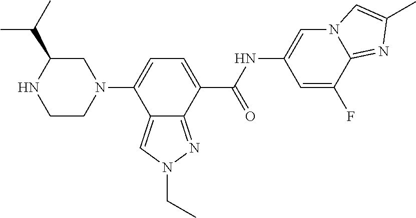 [SMILES: CCn1cc2c(N3CCN[C@@H](C(C)C)C3)ccc(C(=O)Nc3cc(F)c4nc(C)cn4c3)c2n1] |
| 536 | 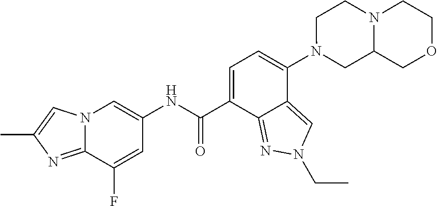 [SMILES: CCn1cc2c(N3CCN4CCOCC4C3)ccc(C(=O)Nc3cc(F)c4nc(C)cn4c3)c2n1] |
| 537 | 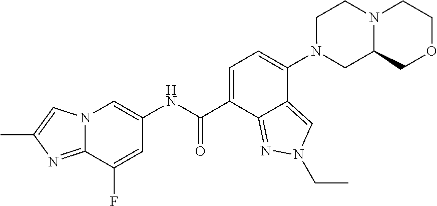 [SMILES: CCn1cc2c(N3CCN4CCOC[C@H]4C3)ccc(C(=O)Nc3cc(F)c4nc(C)cn4c3)c2n1] |
| 538 | 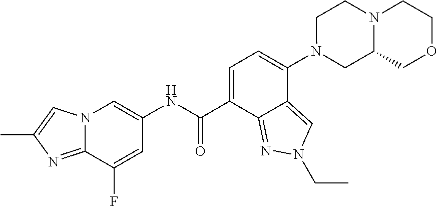 [SMILES: CCn1cc2c(N3CCN4CCOC[C@@H]4C3)ccc(C(=O)Nc3cc(F)c4nc(C)cn4c3)c2n1] |
| 539 | 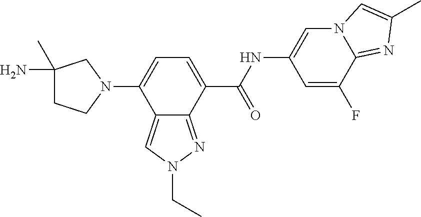 [SMILES: CCn1cc2c(N3CCC(C)(N)C3)ccc(C(=O)Nc3cc(F)c4nc(C)cn4c3)c2n1] |
| 540 | 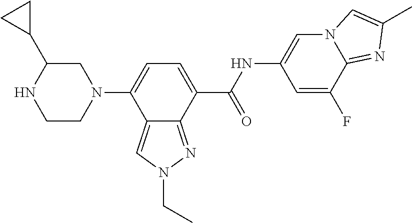 [SMILES: CCn1cc2c(N3CCNC(C4CC4)C3)ccc(C(=O)Nc3cc(F)c4nc(C)cn4c3)c2n1] |
| 541 | 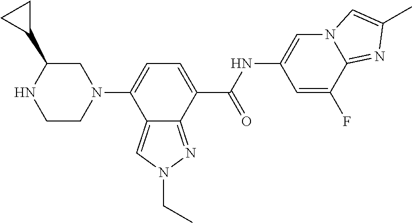 [SMILES: CCn1cc2c(N3CCN[C@@H](C4CC4)C3)ccc(C(=O)Nc3cc(F)c4nc(C)cn4c3)c2n1] |
| 542 | 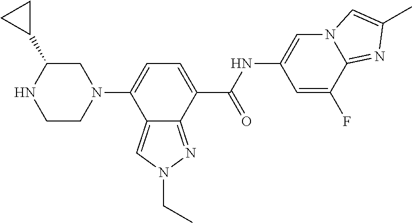 [SMILES: CCn1cc2c(N3CCN[C@H](C4CC4)C3)ccc(C(=O)Nc3cc(F)c4nc(C)cn4c3)c2n1] |
| 543 | 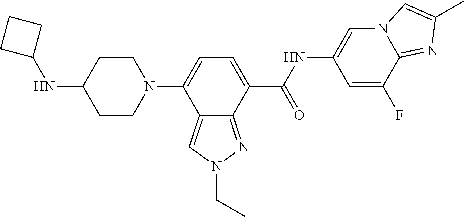 [SMILES: CCn1cc2c(N3CCC(NC4CCC4)CC3)ccc(C(=O)Nc3cc(F)c4nc(C)cn4c3)c2n1] |
| 544 | 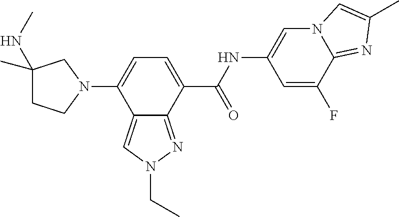 |
| 545 | 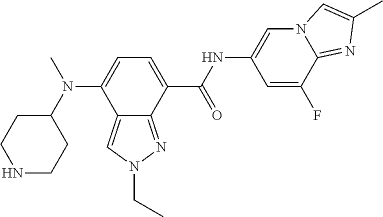 [SMILES: CCn1cc2c(N(C)C3CCNCC3)ccc(C(=O)Nc3cc(F)c4nc(C)cn4c3)c2n1] |
| 546 | 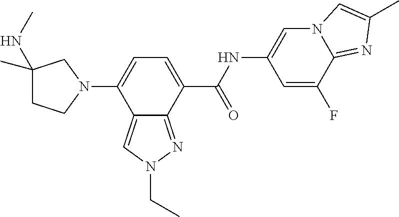 |
| 547 | 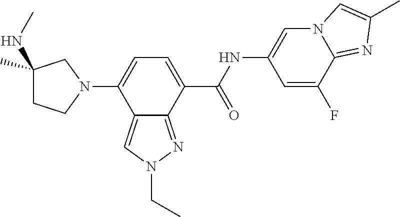 |
| 548 | 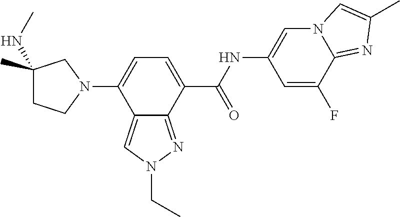 |
| 549 | 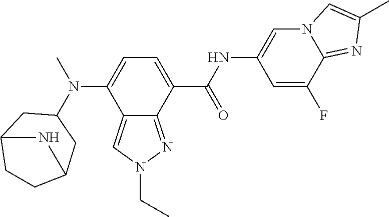 [SMILES: CCn1cc2c(N(C)C3CC4CCC(C3)N4)ccc(C(=O)Nc3cc(F)c4nc(C)cn4c3)c2n1] |
| 550 | 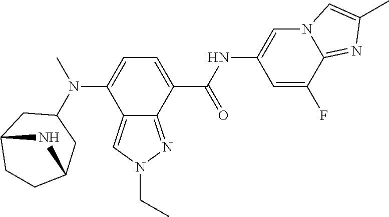 [SMILES: CCn1cc2c(N(C)C3C[C@H]4CC[C@@H](C3)N4)ccc(C(=O)Nc3cc(F)c4nc(C)cn4c3)c2n1] |
| 551 | 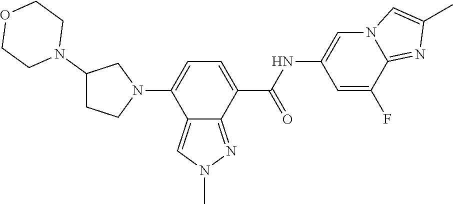 [SMILES: Cc1cn2cc(NC(=O)c3ccc(N4CCC(N5CCOCC5)C4)c4cn(C)nc34)cc(F)c2n1] |
| 552 | 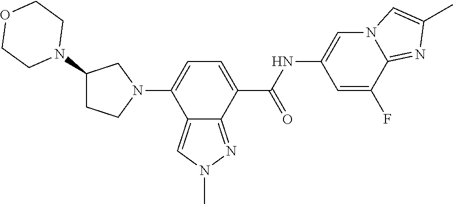 [SMILES: Cc1cn2cc(NC(=O)c3ccc(N4CC[C@@H](N5CCOCC5)C4)c4cn(C)nc34)cc(F)c2n1] |
| 553 | 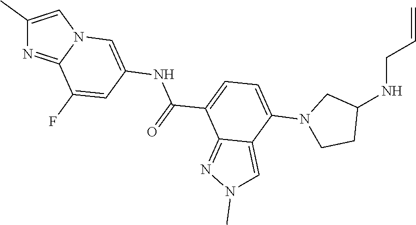 [SMILES: C=CCNC1CCN(c2ccc(C(=O)Nc3cc(F)c4nc(C)cn4c3)c3nn(C)cc23)C1] |
| 554 | 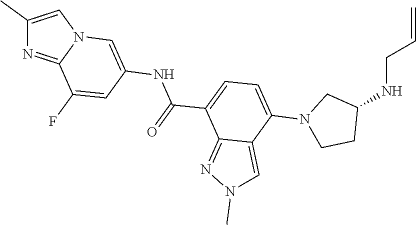 [SMILES: C=CCN[C@@H]1CCN(c2ccc(C(=O)Nc3cc(F)c4nc(C)cn4c3)c3nn(C)cc23)C1] |
| 555 | 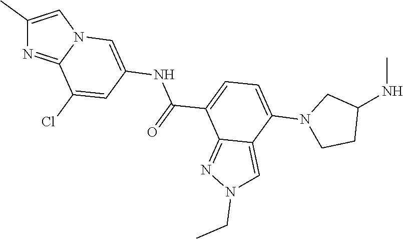 |
| 556 | 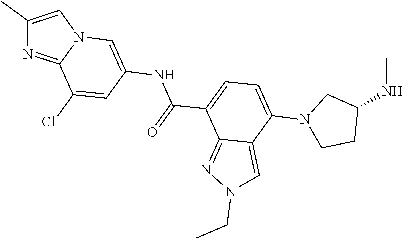 |
| 557 | 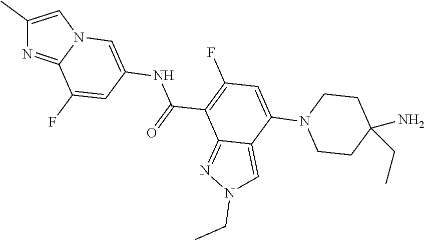 [SMILES: CCn1cc2c(N3CCC(N)(CC)CC3)cc(F)c(C(=O)Nc3cc(F)c4nc(C)cn4c3)c2n1] |
| 558 | 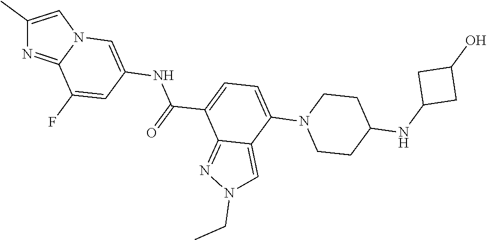 [SMILES: CCn1cc2c(N3CCC(NC4CC(O)C4)CC3)ccc(C(=O)Nc3cc(F)c4nc(C)cn4c3)c2n1] |
| 559 | 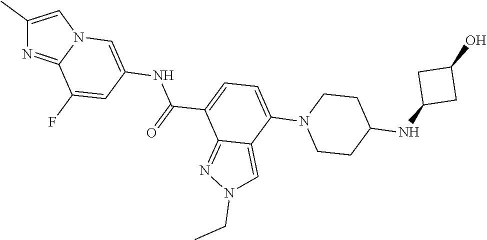 [SMILES: CCn1cc2c(N3CCC(N[C@H]4C[C@@H](O)C4)CC3)ccc(C(=O)Nc3cc(F)c4nc(C)cn4c3)c2n1] |
| 560 | 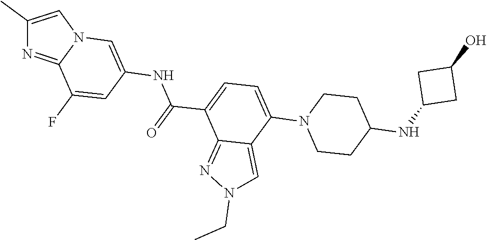 [SMILES: CCn1cc2c(N3CCC(N[C@H]4C[C@H](O)C4)CC3)ccc(C(=O)Nc3cc(F)c4nc(C)cn4c3)c2n1] |
| 561 | 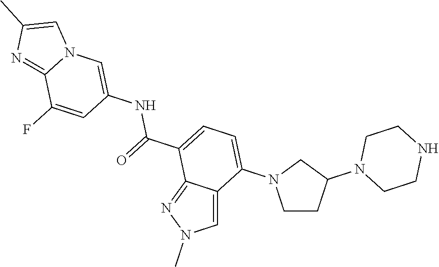 [SMILES: Cc1cn2cc(NC(=O)c3ccc(N4CCC(N5CCNCC5)C4)c4cn(C)nc34)cc(F)c2n1] |
| 562 | 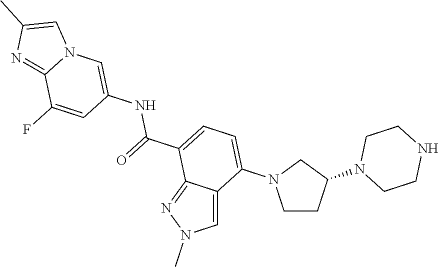 [SMILES: Cc1cn2cc(NC(=O)c3ccc(N4CC[C@@H](N5CCNCC5)C4)c4cn(C)nc34)cc(F)c2n1] |
| 563 | 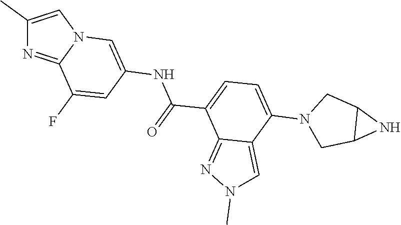 [SMILES: Cc1cn2cc(NC(=O)c3ccc(N4CC5NC5C4)c4cn(C)nc34)cc(F)c2n1] |
| 564 | 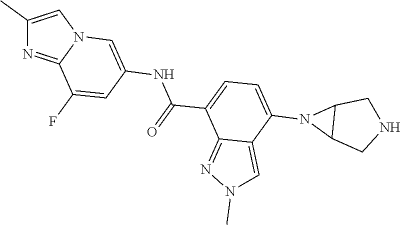 [SMILES: Cc1cn2cc(NC(=O)c3ccc(N4C5CNCC54)c4cn(C)nc34)cc(F)c2n1] |
| 565 | 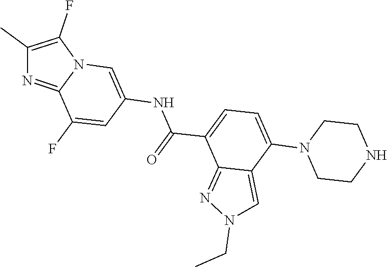 [SMILES: CCn1cc2c(N3CCNCC3)ccc(C(=O)Nc3cc(F)c4nc(C)c(F)n4c3)c2n1] |
| 566 | 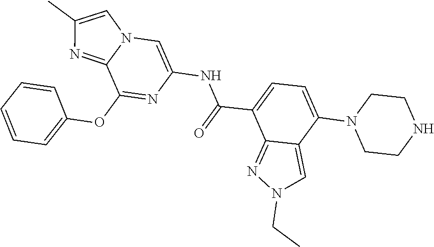 [SMILES: CCn1cc2c(N3CCNCC3)ccc(C(=O)Nc3cn4cc(C)nc4c(Oc4ccccc4)n3)c2n1] |
| 567 | 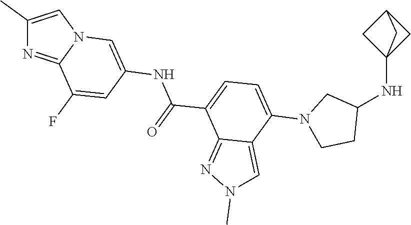 [SMILES: Cc1cn2cc(NC(=O)c3ccc(N4CCC(NC56CC(C5)C6)C4)c4cn(C)nc34)cc(F)c2n1] |
| 568 | 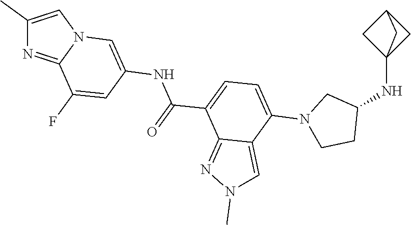 [SMILES: Cc1cn2cc(NC(=O)c3ccc(N4CC[C@@H](NC56CC(C5)C6)C4)c4cn(C)nc34)cc(F)c2n1] |
| 569 | 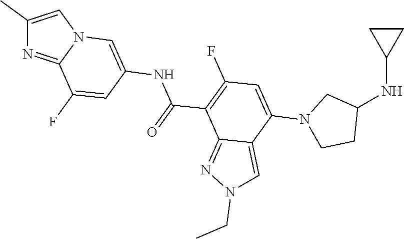 [SMILES: CCn1cc2c(N3CCC(NC4CC4)C3)cc(F)c(C(=O)Nc3cc(F)c4nc(C)cn4c3)c2n1] |
| 570 | 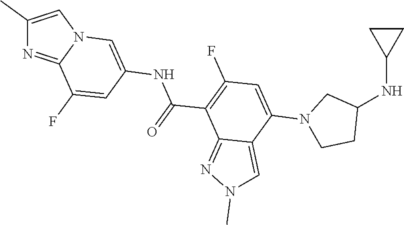 [SMILES: Cc1cn2cc(NC(=O)c3c(F)cc(N4CCC(NC5CC5)C4)c4cn(C)nc34)cc(F)c2n1] |
| 571 | 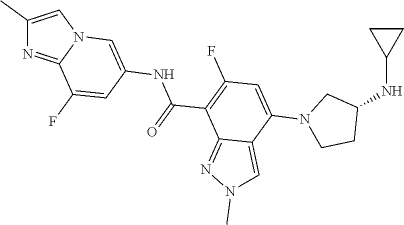 [SMILES: Cc1cn2cc(NC(=O)c3c(F)cc(N4CC[C@@H](NC5CC5)C4)c4cn(C)nc34)cc(F)c2n1] |
| 572 | 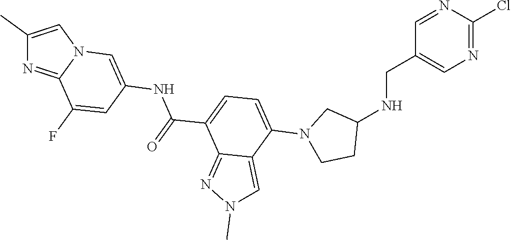 [SMILES: Cc1cn2cc(NC(=O)c3ccc(N4CCC(NCc5cnc(Cl)nc5)C4)c4cn(C)nc34)cc(F)c2n1] |
| 573 | 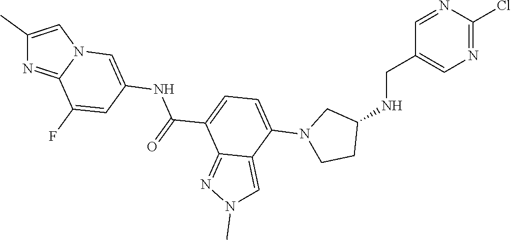 [SMILES: Cc1cn2cc(NC(=O)c3ccc(N4CC[C@@H](NCc5cnc(Cl)nc5)C4)c4cn(C)nc34)cc(F)c2n1] |
| 574 | 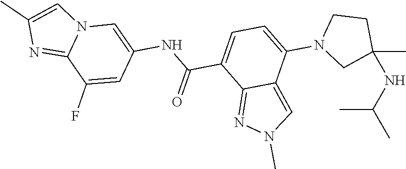 |
| 575 | 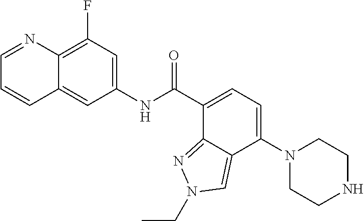 [SMILES: CCn1cc2c(N3CCNCC3)ccc(C(=O)Nc3cc(F)c4ncccc4c3)c2n1] |
| 576 | 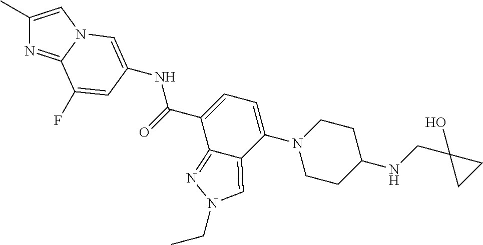 [SMILES: CCn1cc2c(N3CCC(NCC4(O)CC4)CC3)ccc(C(=O)Nc3cc(F)c4nc(C)cn4c3)c2n1] |
| 577 | 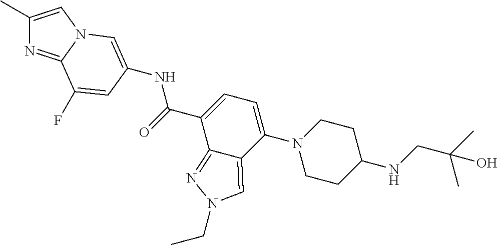 [SMILES: CCn1cc2c(N3CCC(NCC(C)(C)O)CC3)ccc(C(=O)Nc3cc(F)c4nc(C)cn4c3)c2n1] |
| 578 | 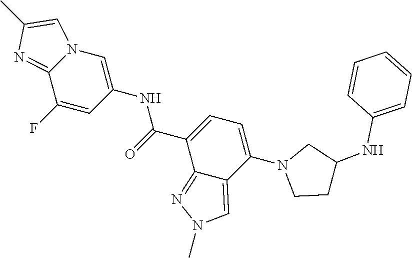 [SMILES: Cc1cn2cc(NC(=O)c3ccc(N4CCC(Nc5ccccc5)C4)c4cn(C)nc34)cc(F)c2n1] |
| 579 | 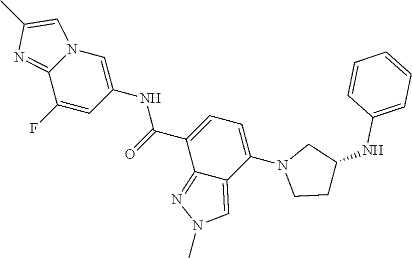 [SMILES: Cc1cn2cc(NC(=O)c3ccc(N4CC[C@@H](Nc5ccccc5)C4)c4cn(C)nc34)cc(F)c2n1] |
| 580 | 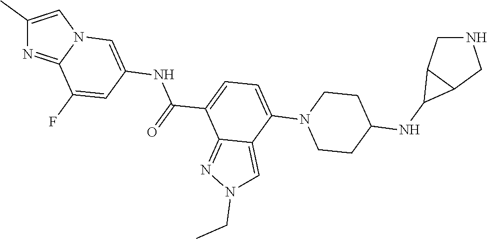 [SMILES: CCn1cc2c(N3CCC(NC4C5CNCC54)CC3)ccc(C(=O)Nc3cc(F)c4nc(C)cn4c3)c2n1] |
| 581 | 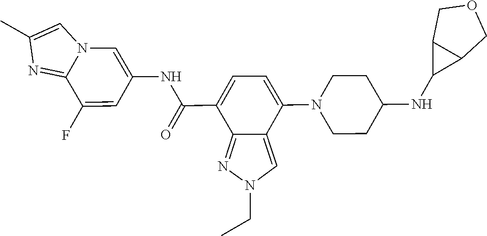 [SMILES: CCn1cc2c(N3CCC(NC4C5COCC54)CC3)ccc(C(=O)Nc3cc(F)c4nc(C)cn4c3)c2n1] |
| 582 | 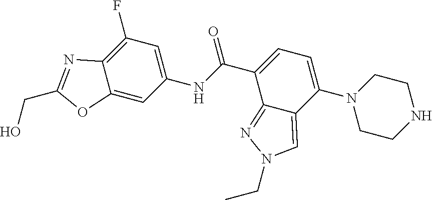 [SMILES: CCn1cc2c(N3CCNCC3)ccc(C(=O)Nc3cc(F)c4nc(CO)oc4c3)c2n1] |
| 583 | 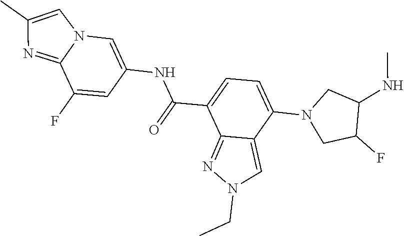 [SMILES: CCn1cc2c(N3CC(F)C(NC)C3)ccc(C(=O)Nc3cc(F)c4nc(C)cn4c3)c2n1] |
| 584 | 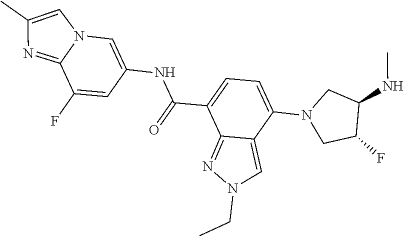 [SMILES: CCn1cc2c(N3C[C@@H](F)[C@H](NC)C3)ccc(C(=O)Nc3cc(F)c4nc(C)cn4c3)c2n1] |
| 585 | 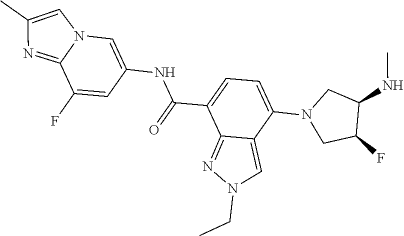 [SMILES: CCn1cc2c(N3C[C@H](F)[C@H](NC)C3)ccc(C(=O)Nc3cc(F)c4nc(C)cn4c3)c2n1] |
| 586 | 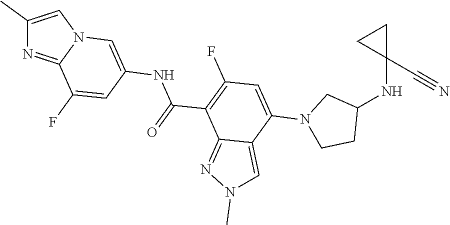 [SMILES: Cc1cn2cc(NC(=O)c3c(F)cc(N4CCC(NC5(C#N)CC5)C4)c4cn(C)nc34)cc(F)c2n1] |
| 587 | 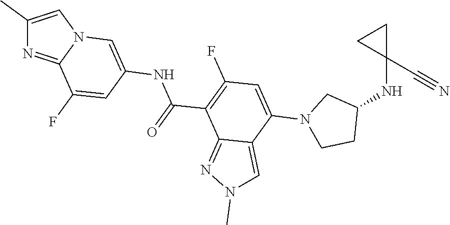 [SMILES: Cc1cn2cc(NC(=O)c3c(F)cc(N4CC[C@@H](NC5(C#N)CC5)C4)c4cn(C)nc34)cc(F)c2n1] |
| 588 | 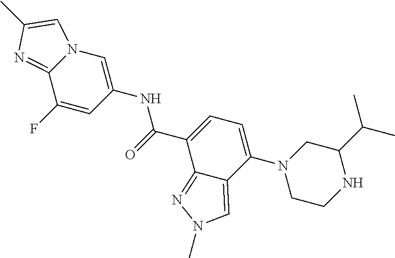 [SMILES: Cc1cn2cc(NC(=O)c3ccc(N4CCNC(C(C)C)C4)c4cn(C)nc34)cc(F)c2n1] |
| 589 | 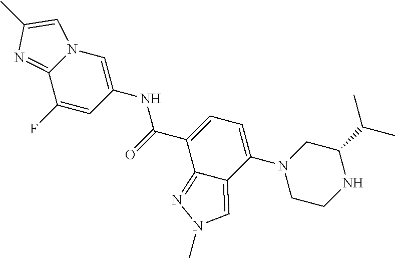 [SMILES: Cc1cn2cc(NC(=O)c3ccc(N4CCN[C@@H](C(C)C)C4)c4cn(C)nc34)cc(F)c2n1] |
| 590 | 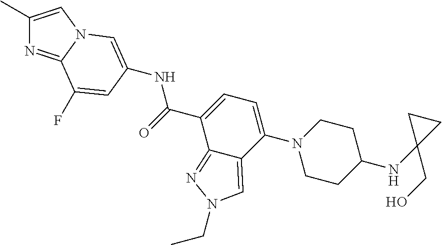 [SMILES: CCn1cc2c(N3CCC(NC4(CO)CC4)CC3)ccc(C(=O)Nc3cc(F)c4nc(C)cn4c3)c2n1] |
| 591 | 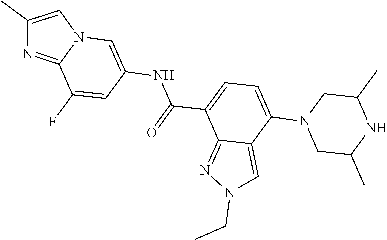 |
| 592 | 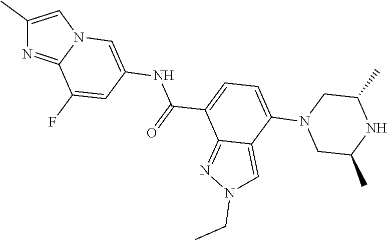 |
| 593 | 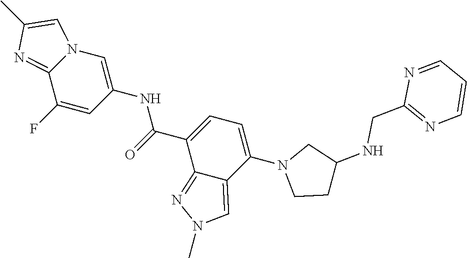 [SMILES: Cc1cn2cc(NC(=O)c3ccc(N4CCC(NCc5ncccn5)C4)c4cn(C)nc34)cc(F)c2n1] |
| 594 | 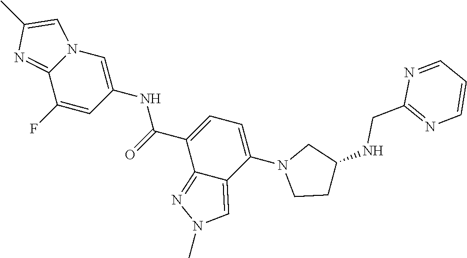 [SMILES: Cc1cn2cc(NC(=O)c3ccc(N4CC[C@@H](NCc5ncccn5)C4)c4cn(C)nc34)cc(F)c2n1] |
| 595 | 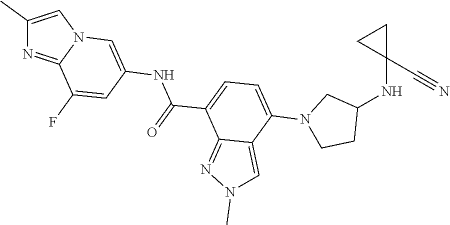 [SMILES: Cc1cn2cc(NC(=O)c3ccc(N4CCC(NC5(C#N)CC5)C4)c4cn(C)nc34)cc(F)c2n1] |
| 596 | 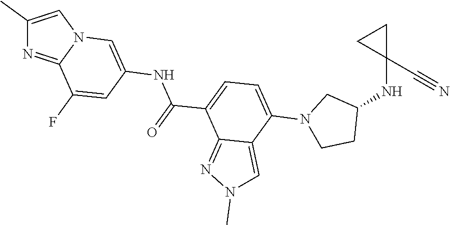 [SMILES: Cc1cn2cc(NC(=O)c3ccc(N4CC[C@@H](NC5(C#N)CC5)C4)c4cn(C)nc34)cc(F)c2n1] |
| 597 | 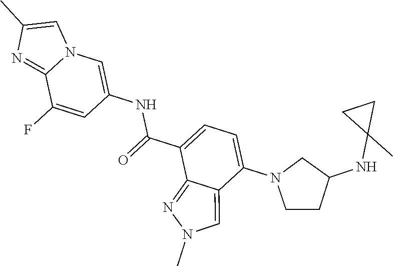 |
| 598 | 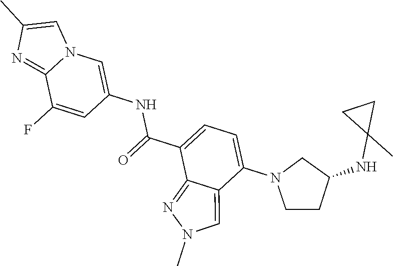 |
| 599 | 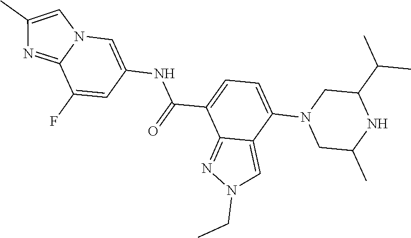 [SMILES: CCn1cc2c(N3CC(C)NC(C(C)C)C3)ccc(C(=O)Nc3cc(F)c4nc(C)cn4c3)c2n1] |
| 600 | 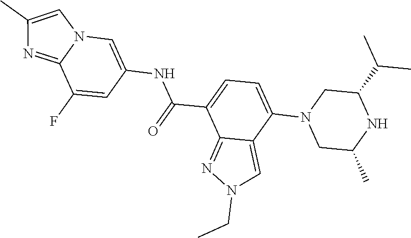 [SMILES: CCn1cc2c(N3C[C@@H](C)N[C@@H](C(C)C)C3)ccc(C(=O)Nc3cc(F)c4nc(C)cn4c3)c2n1] |
| 601 | 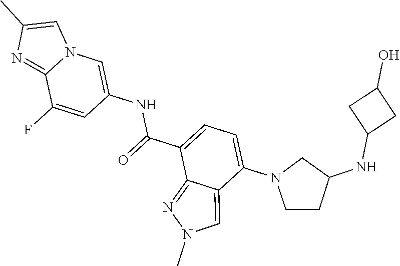 [SMILES: Cc1cn2cc(NC(=O)c3ccc(N4CCC(NC5CC(O)C5)C4)c4cn(C)nc34)cc(F)c2n1] |
| 602 | 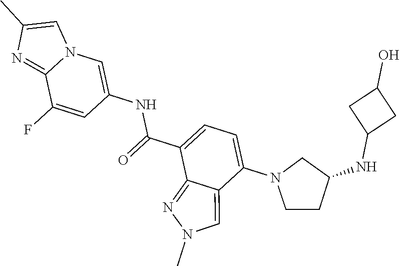 [SMILES: Cc1cn2cc(NC(=O)c3ccc(N4CC[C@@H](NC5CC(O)C5)C4)c4cn(C)nc34)cc(F)c2n1] |
| 603 | 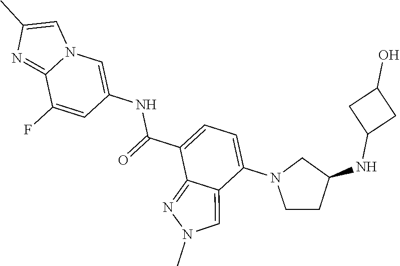 [SMILES: Cc1cn2cc(NC(=O)c3ccc(N4CC[C@H](NC5CC(O)C5)C4)c4cn(C)nc34)cc(F)c2n1] |
| 604 | 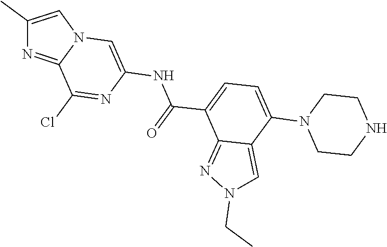 [SMILES: CCn1cc2c(N3CCNCC3)ccc(C(=O)Nc3cn4cc(C)nc4c(Cl)n3)c2n1] |
| 605 | 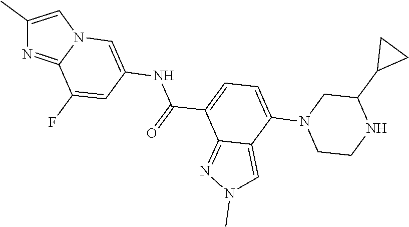 [SMILES: Cc1cn2cc(NC(=O)c3ccc(N4CCNC(C5CC5)C4)c4cn(C)nc34)cc(F)c2n1] |
| 606 | 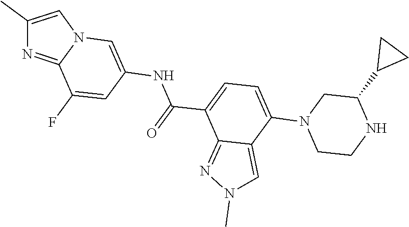 [SMILES: Cc1cn2cc(NC(=O)c3ccc(N4CCN[C@@H](C5CC5)C4)c4cn(C)nc34)cc(F)c2n1] |
| 607 | 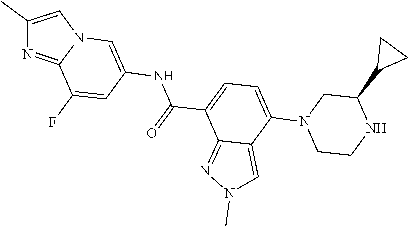 [SMILES: Cc1cn2cc(NC(=O)c3ccc(N4CCN[C@H](C5CC5)C4)c4cn(C)nc34)cc(F)c2n1] |
| 608 | 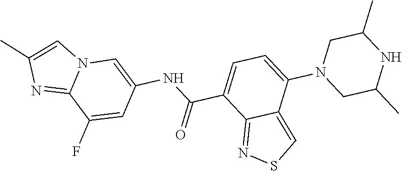 |
| 609 | 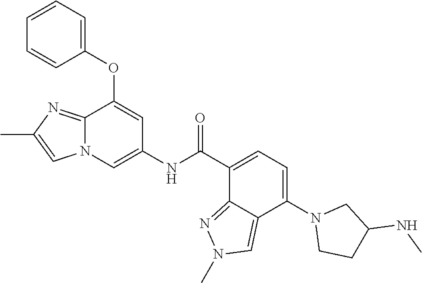 |
| 610 | 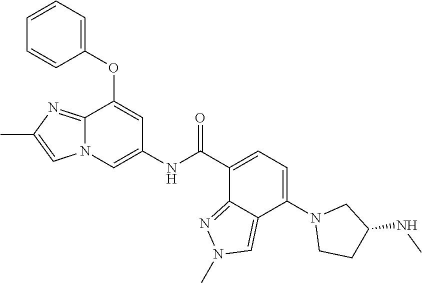 |
| 611 | 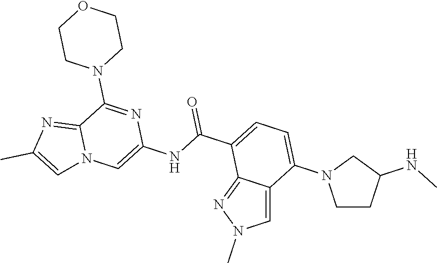 |
| 612 | 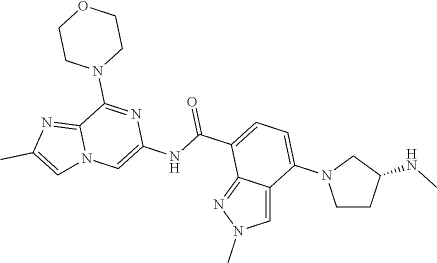 |
| 613 | 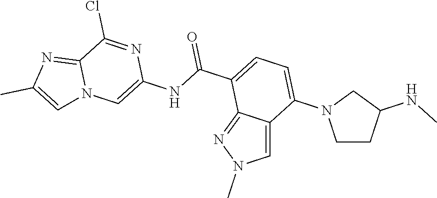 |
| 614 | 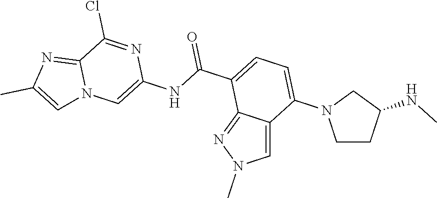 |
| 615 | 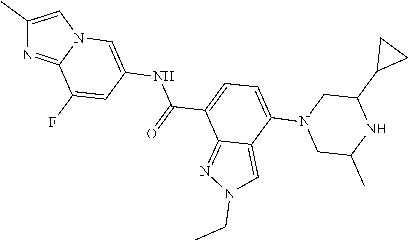 [SMILES: CCn1cc2c(N3CC(C)NC(C4CC4)C3)ccc(C(=O)Nc3cc(F)c4nc(C)cn4c3)c2n1] |
| 616 | 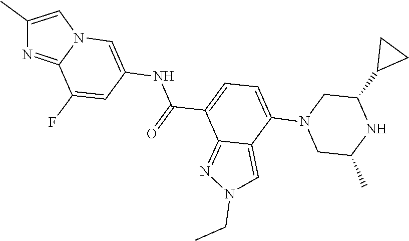 [SMILES: CCn1cc2c(N3C[C@@H](C)N[C@@H](C4CC4)C3)ccc(C(=O)Nc3cc(F)c4nc(C)cn4c3)c2n1] |
| 617 | 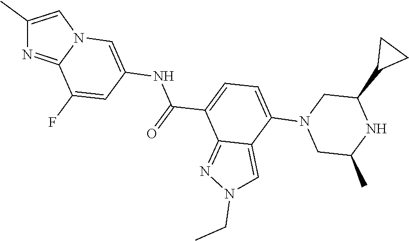 [SMILES: CCn1cc2c(N3C[C@@H](C4CC4)N[C@@H](C)C3)ccc(C(=O)Nc3cc(F)c4nc(C)cn4c3)c2n1] |
| 618 | 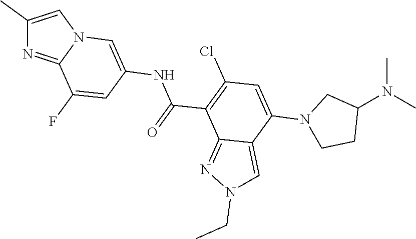 |
| 619 | 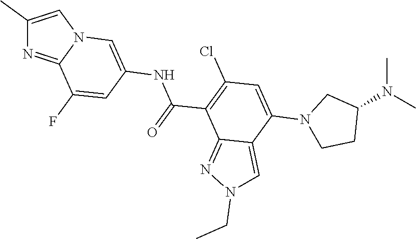 |
| 620 | 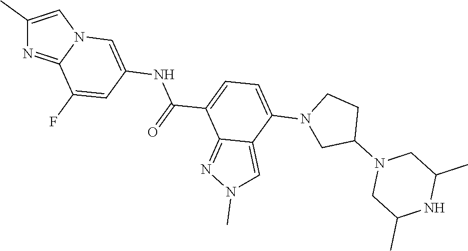 |
| 621 | 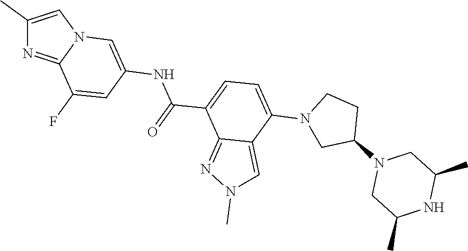 |
| 622 | 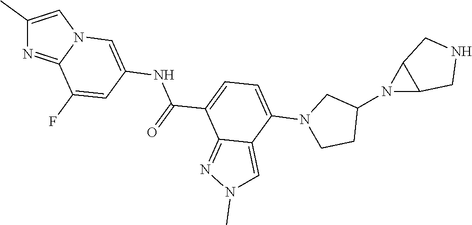 [SMILES: Cc1cn2cc(NC(=O)c3ccc(N4CCC(N5C6CNCC65)C4)c4cn(C)nc34)cc(F)c2n1] |
| 623 | 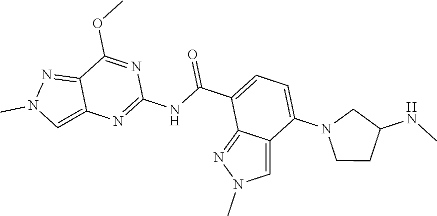 |
| 624 | 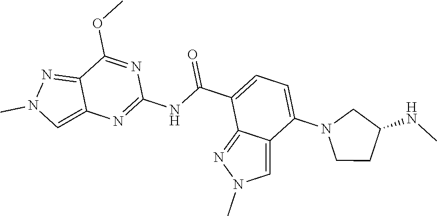 |
| 625 | 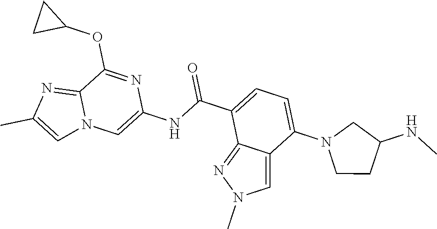 |
| 626 | 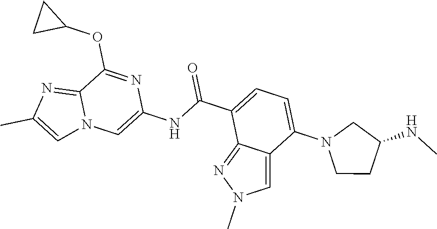 |
| 627 | 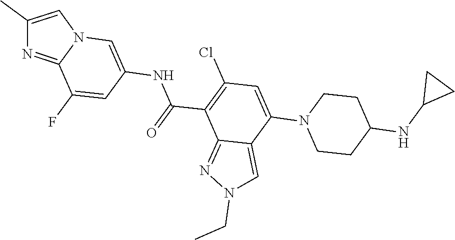 [SMILES: CCn1cc2c(N3CCC(NC4CC4)CC3)cc(Cl)c(C(=O)Nc3cc(F)c4nc(C)cn4c3)c2n1] |
| 628 | 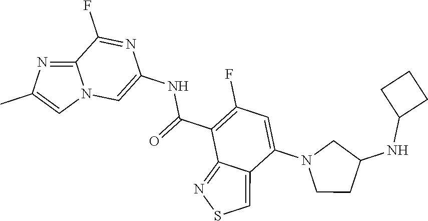 [SMILES: Cc1cn2cc(NC(=O)c3c(F)cc(N4CCC(NC5CCC5)C4)c4csnc34)nc(F)c2n1] |
| 629 | 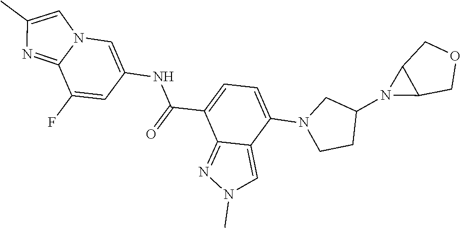 [SMILES: Cc1cn2cc(NC(=O)c3ccc(N4CCC(N5C6COCC65)C4)c4cn(C)nc34)cc(F)c2n1] |
| 630 | 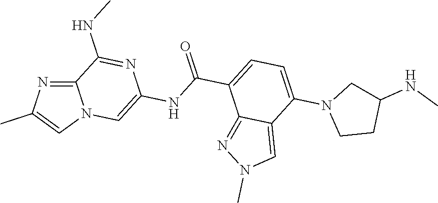 |
| 631 | 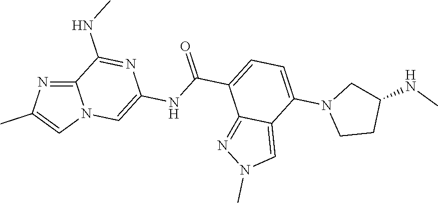 |
| 632 | 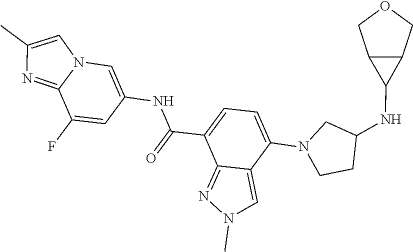 [SMILES: Cc1cn2cc(NC(=O)c3ccc(N4CCC(NC5C6COCC65)C4)c4cn(C)nc34)cc(F)c2n1] |
| 633 | 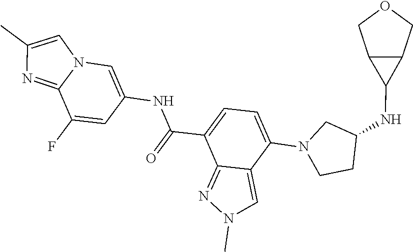 [SMILES: Cc1cn2cc(NC(=O)c3ccc(N4CC[C@@H](NC5C6COCC65)C4)c4cn(C)nc34)cc(F)c2n1] |
| 634 | 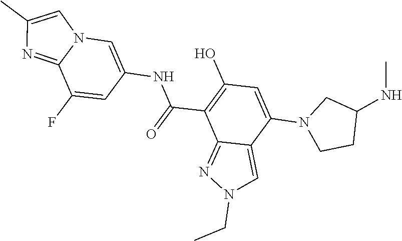 |
| 635 | 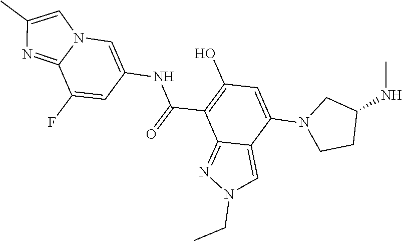 |
| 636 | 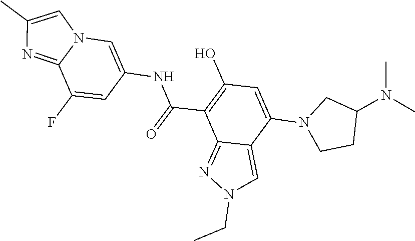 |
| 637 | 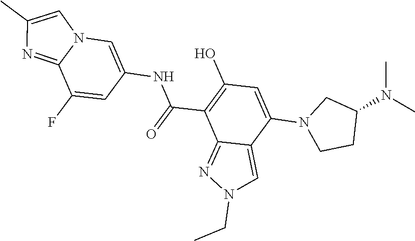 |
| 638 | 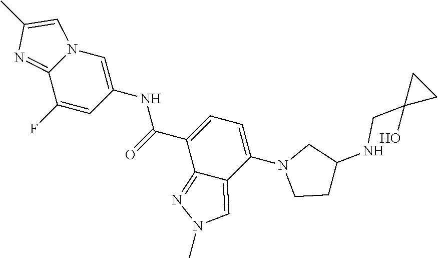 [SMILES: Cc1cn2cc(NC(=O)c3ccc(N4CCC(NCC5(O)CC5)C4)c4cn(C)nc34)cc(F)c2n1] |
| 639 | 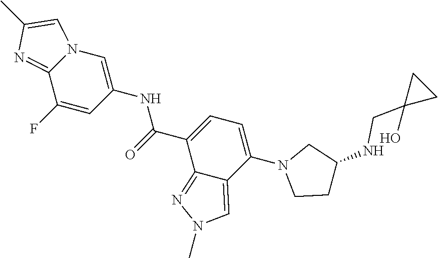 [SMILES: Cc1cn2cc(NC(=O)c3ccc(N4CC[C@@H](NCC5(O)CC5)C4)c4cn(C)nc34)cc(F)c2n1] |
| 640 | 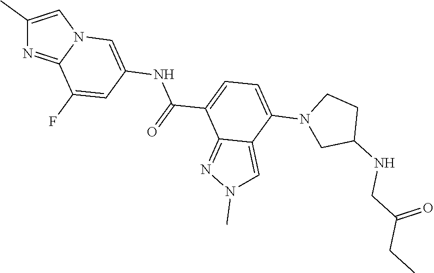 [SMILES: CCC(=O)CNC1CCN(c2ccc(C(=O)Nc3cc(F)c4nc(C)cn4c3)c3nn(C)cc23)C1] |
| 641 | 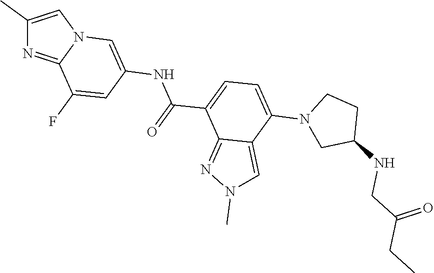 [SMILES: CCC(=O)CN[C@@H]1CCN(c2ccc(C(=O)Nc3cc(F)c4nc(C)cn4c3)c3nn(C)cc23)C1] |
| 642 | 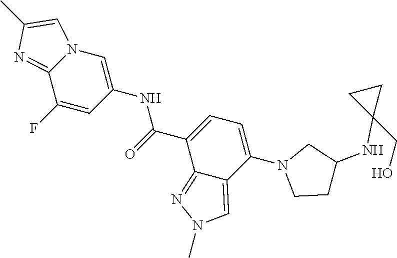 [SMILES: Cc1cn2cc(NC(=O)c3ccc(N4CCC(NC5(CO)CC5)C4)c4cn(C)nc34)cc(F)c2n1] |
| 643 | 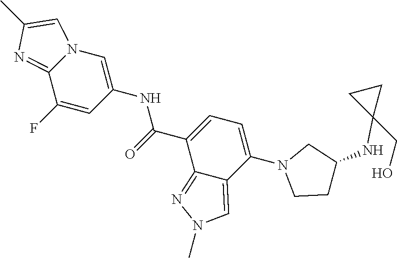 [SMILES: Cc1cn2cc(NC(=O)c3ccc(N4CC[C@@H](NC5(CO)CC5)C4)c4cn(C)nc34)cc(F)c2n1] |
| 644 | 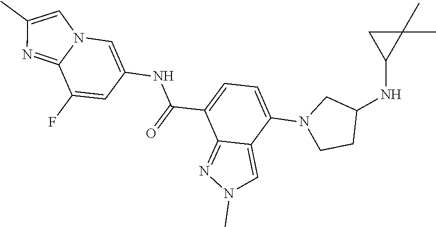 |
| 645 | 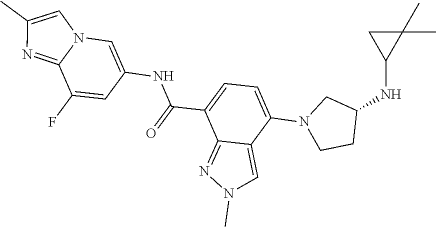 |
| 646 | 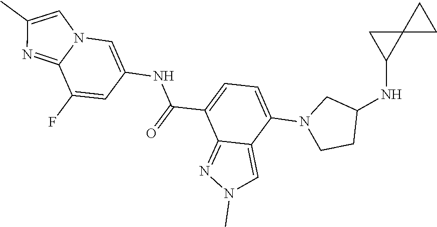 [SMILES: Cc1cn2cc(NC(=O)c3ccc(N4CCC(NC5CC56CC6)C4)c4cn(C)nc34)cc(F)c2n1] |
| 647 | 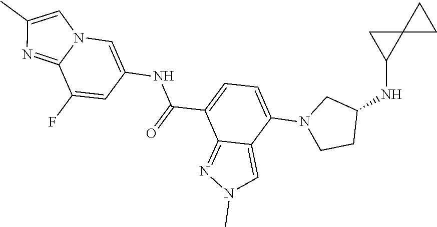 [SMILES: Cc1cn2cc(NC(=O)c3ccc(N4CC[C@@H](NC5CC56CC6)C4)c4cn(C)nc34)cc(F)c2n1] |
| 648 | 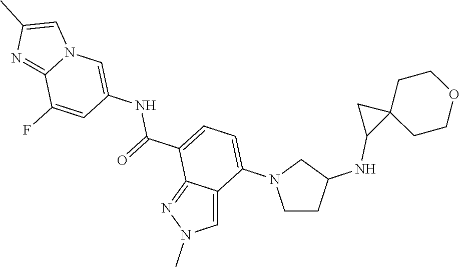 [SMILES: Cc1cn2cc(NC(=O)c3ccc(N4CCC(NC5CC56CCOCC6)C4)c4cn(C)nc34)cc(F)c2n1] |
| 649 | 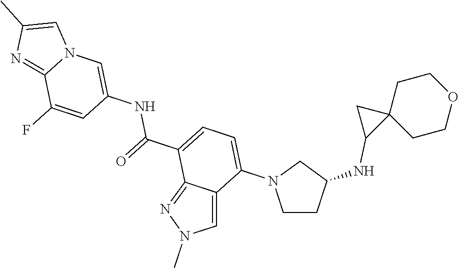 [SMILES: Cc1cn2cc(NC(=O)c3ccc(N4CC[C@@H](NC5CC56CCOCC6)C4)c4cn(C)nc34)cc(F)c2n1] |
| 650 | 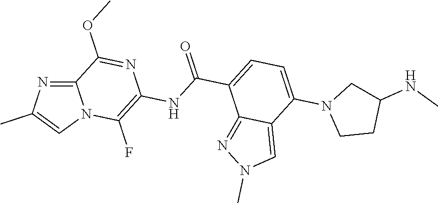 |
| 651 | 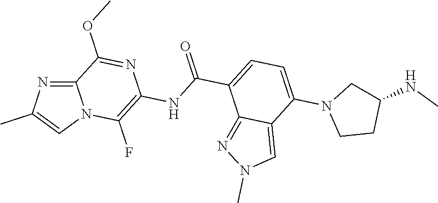 |
| 652 | 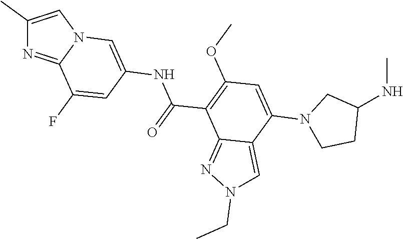 |
| 653 | 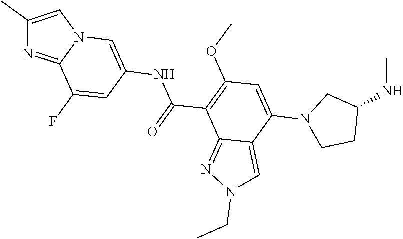 |
| 654 | 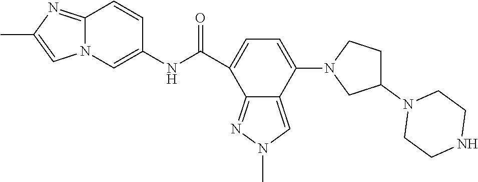 [SMILES: Cc1cn2cc(NC(=O)c3ccc(N4CCC(N5CCNCC5)C4)c4cn(C)nc34)ccc2n1] |
| 656 | 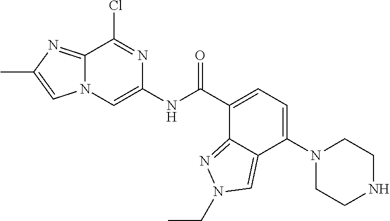 [SMILES: CCn1cc2c(N3CCNCC3)ccc(C(=O)Nc3cn4cc(C)nc4c(Cl)n3)c2n1] |
| 657 | 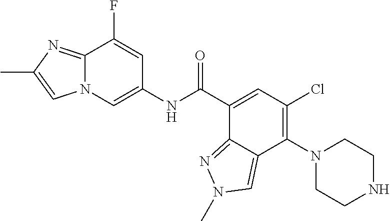 [SMILES: Cc1cn2cc(NC(=O)c3cc(Cl)c(N4CCNCC4)c4cn(C)nc34)cc(F)c2n1] |
| 658 | 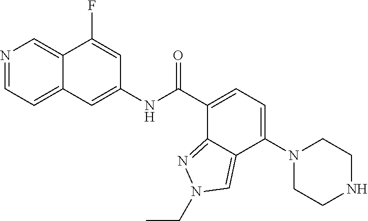 [SMILES: CCn1cc2c(N3CCNCC3)ccc(C(=O)Nc3cc(F)c4cnccc4c3)c2n1] |
| 659 | 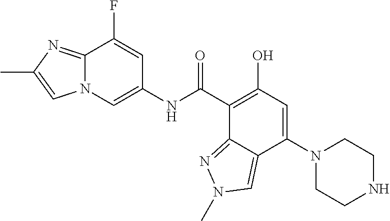 [SMILES: Cc1cn2cc(NC(=O)c3c(O)cc(N4CCNCC4)c4cn(C)nc34)cc(F)c2n1] |
| 660 | 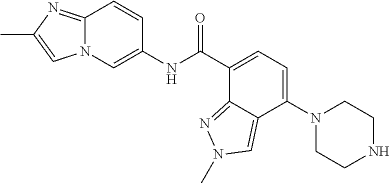 [SMILES: Cc1cn2cc(NC(=O)c3ccc(N4CCNCC4)c4cn(C)nc34)ccc2n1] |
| 661 | 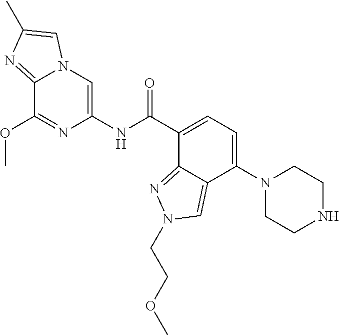 [SMILES: COCCn1cc2c(N3CCNCC3)ccc(C(=O)Nc3cn4cc(C)nc4c(OC)n3)c2n1] |
| 664 | 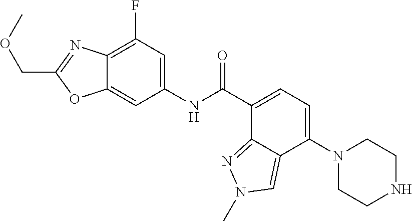 [SMILES: COCc1nc2c(F)cc(NC(=O)c3ccc(N4CCNCC4)c4cn(C)nc34)cc2o1] |
| 665 | 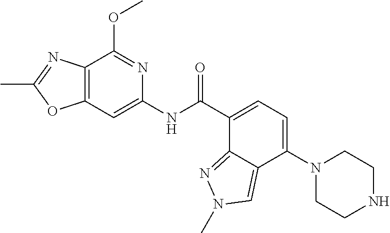 [SMILES: COc1nc(NC(=O)c2ccc(N3CCNCC3)c3cn(C)nc23)cc2oc(C)nc12] |
| 666 | 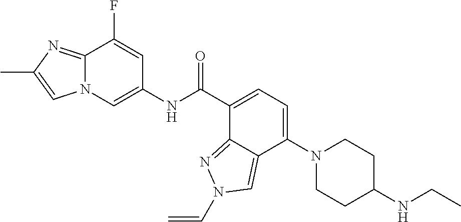 [SMILES: C=Cn1cc2c(N3CCC(NCC)CC3)ccc(C(=O)Nc3cc(F)c4nc(C)cn4c3)c2n1] |
| 667 | 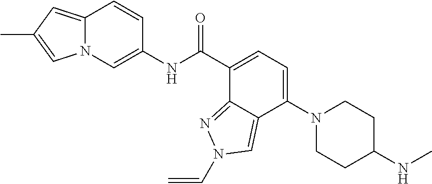 |
| 668 | 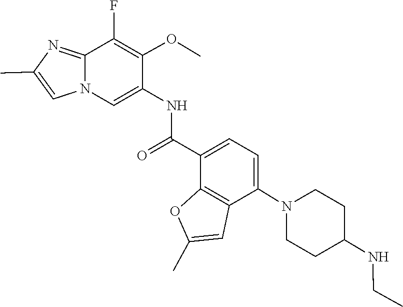 [SMILES: CCNC1CCN(c2ccc(C(=O)Nc3cn4cc(C)nc4c(F)c3OC)c3oc(C)cc23)CC1] |
| 669 | 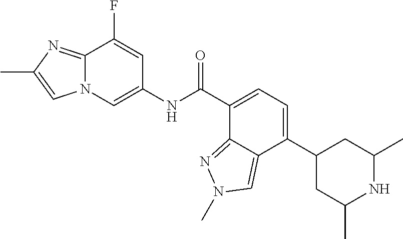 |
| 670 | 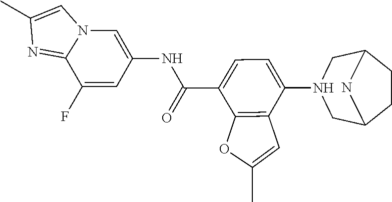 [SMILES: Cc1cn2cc(NC(=O)c3ccc(N4CC5CCC(C4)N5)c4cc(C)oc34)cc(F)c2n1] |
| 671 | 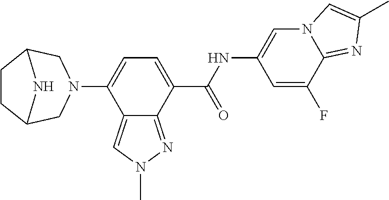 [SMILES: Cc1cn2cc(NC(=O)c3ccc(N4CC5CCC(C4)N5)c4cn(C)nc34)cc(F)c2n1] |
| 672 | 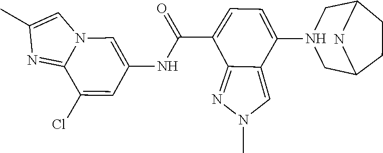 [SMILES: Cc1cn2cc(NC(=O)c3ccc(N4CC5CCC(C4)N5)c4cn(C)nc34)cc(Cl)c2n1] |
| 673 | 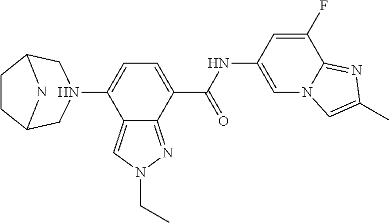 [SMILES: CCn1cc2c(N3CC4CCC(C3)N4)ccc(C(=O)Nc3cc(F)c4nc(C)cn4c3)c2n1] |
| 674 | 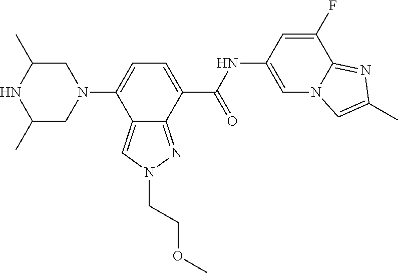 [SMILES: COCCn1cc2c(N3CC(C)NC(C)C3)ccc(C(=O)Nc3cc(F)c4nc(C)cn4c3)c2n1] |
| 675 | 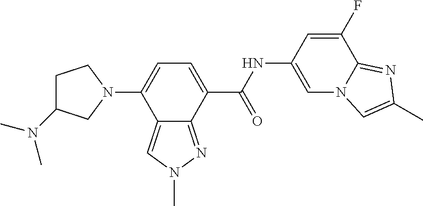 |
| 676 | 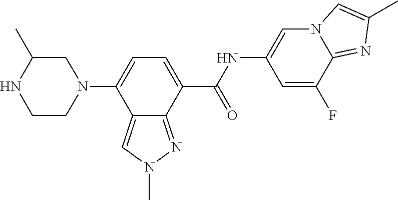 [SMILES: Cc1cn2cc(NC(=O)c3ccc(N4CCNC(C)C4)c4cn(C)nc34)cc(F)c2n1] |
| 677 | 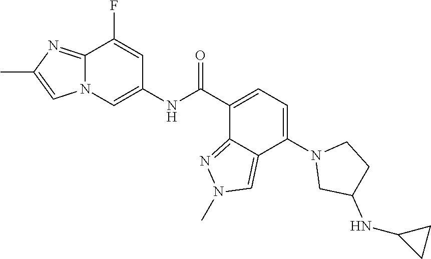 [SMILES: Cc1cn2cc(NC(=O)c3ccc(N4CCC(NC5CC5)C4)c4cn(C)nc34)cc(F)c2n1] |
| 678 | 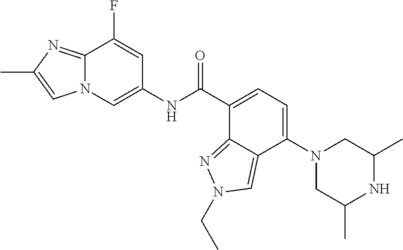 |
| 679 | 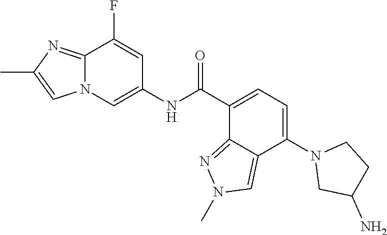 [SMILES: Cc1cn2cc(NC(=O)c3ccc(N4CCC(N)C4)c4cn(C)nc34)cc(F)c2n1] |
| 680 | 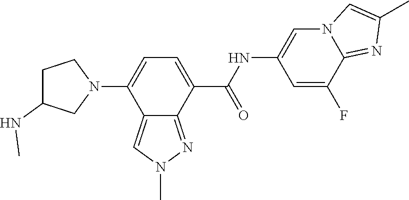 [SMILES: CNC1CCN(c2ccc(C(=O)Nc3cc(F)c4nc(C)cn4c3)c3nn(C)cc23)C1] |
| 681 | 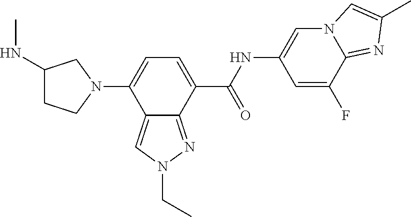 [SMILES: CCn1cc2c(N3CCC(NC)C3)ccc(C(=O)Nc3cc(F)c4nc(C)cn4c3)c2n1] |
| 682 | 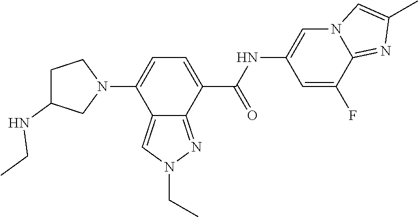 [SMILES: CCNC1CCN(c2ccc(C(=O)Nc3cc(F)c4nc(C)cn4c3)c3nn(CC)cc23)C1] |
| 683 | 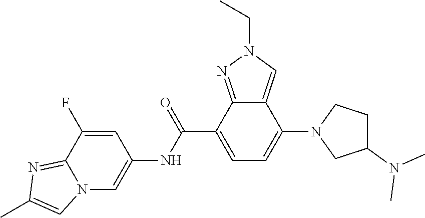 |
| 684 | 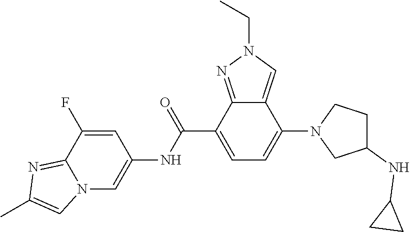 [SMILES: CCn1cc2c(N3CCC(NC4CC4)C3)ccc(C(=O)Nc3cc(F)c4nc(C)cn4c3)c2n1] |
| 685 | 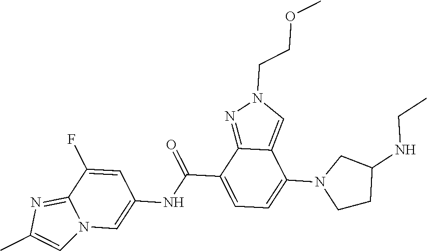 [SMILES: CCNC1CCN(c2ccc(C(=O)Nc3cc(F)c4nc(C)cn4c3)c3nn(CCOC)cc23)C1] |
| 686 | 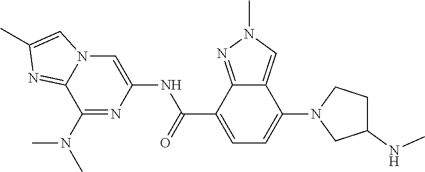 |
| 687 | 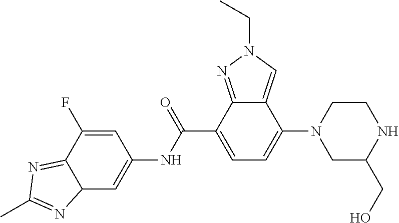 [SMILES: CCn1cc2c(N3CCNC(CO)C3)ccc(C(=O)NC3=CC4N=C(C)N=C4C(F)=C3)c2n1] |
| 688 | 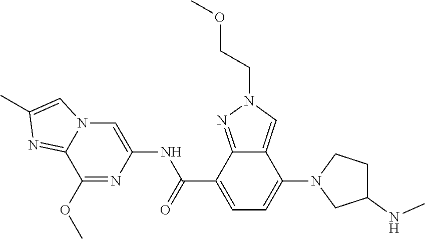 |
| 689 | 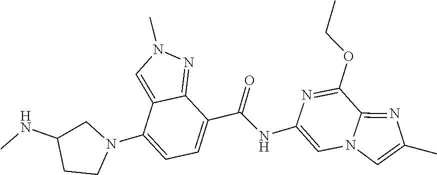 |
| 690 | 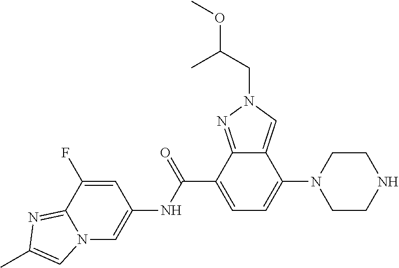 [SMILES: COC(C)Cn1cc2c(N3CCNCC3)ccc(C(=O)Nc3cc(F)c4nc(C)cn4c3)c2n1] |
| 691 | 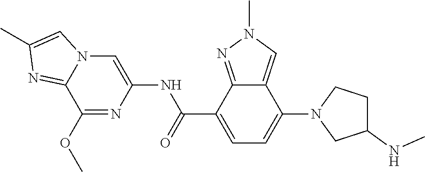 |
| 692 | 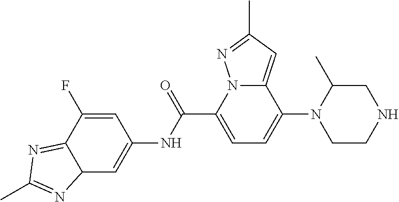 [SMILES: CC1=NC2C=C(NC(=O)c3ccc(N4CCNCC4C)c4cc(C)nn34)C=C(F)C2=N1] |
| 693 | 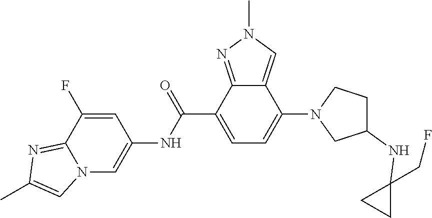 [SMILES: Cc1cn2cc(NC(=O)c3ccc(N4CCC(NC5(CF)CC5)C4)c4cn(C)nc34)cc(F)c2n1] |
| 694 | 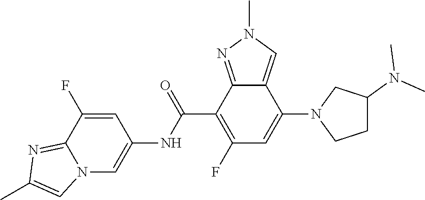 |
| 695 | 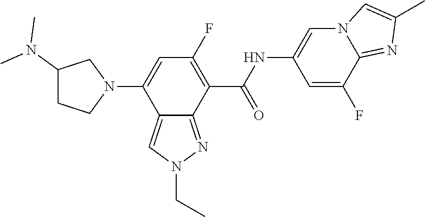 |
| 696 | 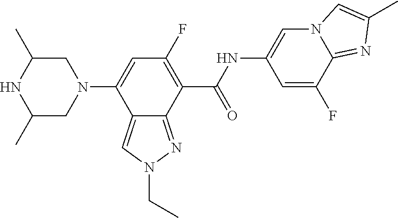 [SMILES: CCn1cc2c(N3CC(C)NC(C)C3)cc(F)c(C(=O)Nc3cc(F)c4nc(C)cn4c3)c2n1] |
| 697 | 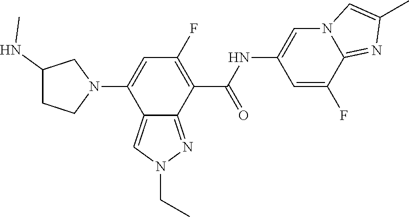 [SMILES: CCn1cc2c(N3CCC(NC)C3)cc(F)c(C(=O)Nc3cc(F)c4nc(C)cn4c3)c2n1] |
| 698 | 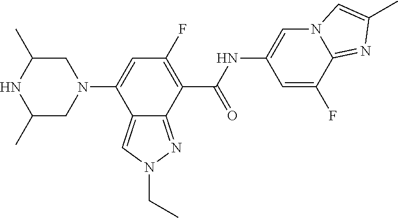 [SMILES: CCn1cc2c(N3CC(C)NC(C)C3)cc(F)c(C(=O)Nc3cc(F)c4nc(C)cn4c3)c2n1] |
| 699 | 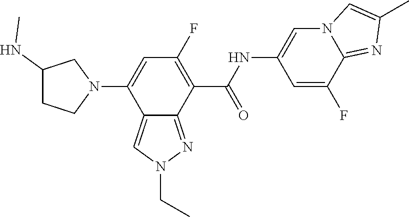 [SMILES: CCn1cc2c(N3CCC(NC)C3)cc(F)c(C(=O)Nc3cc(F)c4nc(C)cn4c3)c2n1] |
| 700 | 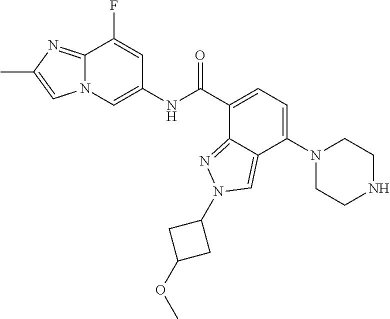 [SMILES: COC1CC(n2cc3c(N4CCNCC4)ccc(C(=O)Nc4cc(F)c5nc(C)cn5c4)c3n2)C1] |
| 701 | 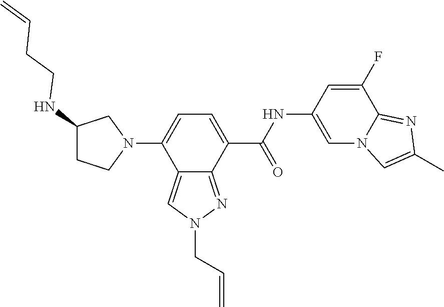 [SMILES: C=CCCN[C@@H]1CCN(c2ccc(C(=O)Nc3cc(F)c4nc(C)cn4c3)c3nn(CC=C)cc23)C1] |
| 702 | 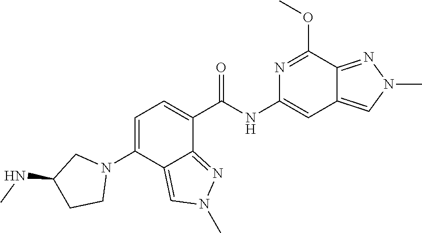 |
| 703 | 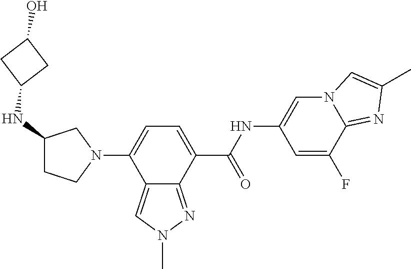 [SMILES: Cc1cn2cc(NC(=O)c3ccc(N4CC[C@@H](N[C@H]5C[C@@H](O)C5)C4)c4cn(C)nc34)cc(F)c2n1] |
| 704 | 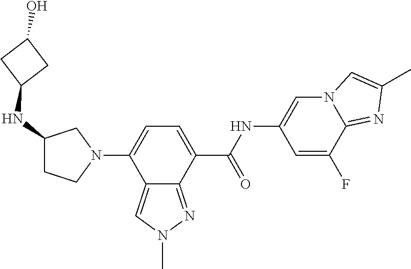 [SMILES: Cc1cn2cc(NC(=O)c3ccc(N4CC[C@@H](N[C@H]5C[C@H](O)C5)C4)c4cn(C)nc34)cc(F)c2n1] |
| 705 | 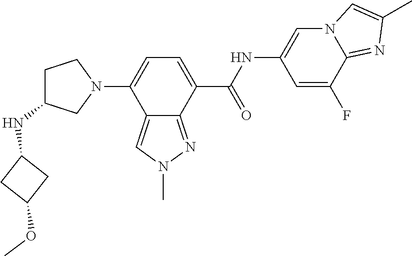 [SMILES: CO[C@H]1C[C@@H](N[C@@H]2CCN(c3ccc(C(=O)Nc4cc(F)c5nc(C)cn5c4)c4nn(C)cc34)C2)C1] |
| 706 | 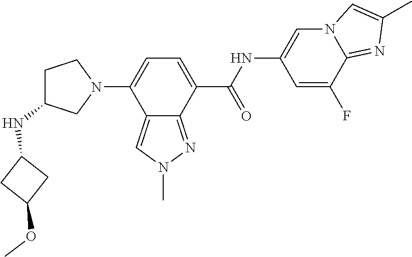 [SMILES: CO[C@H]1C[C@H](N[C@@H]2CCN(c3ccc(C(=O)Nc4cc(F)c5nc(C)cn5c4)c4nn(C)cc34)C2)C1] |
| 707 | 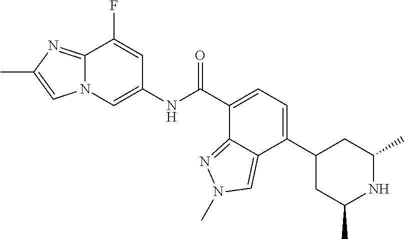 |
| 708 | 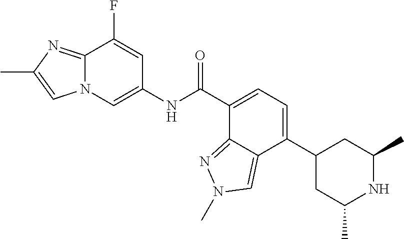 |
| 709 | 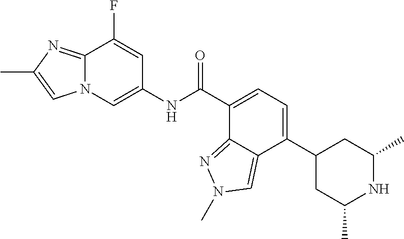 |
| 710 | 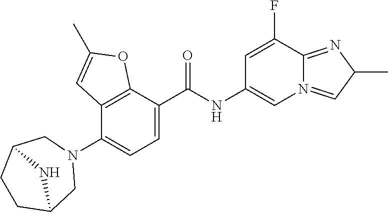 |
| 711 | 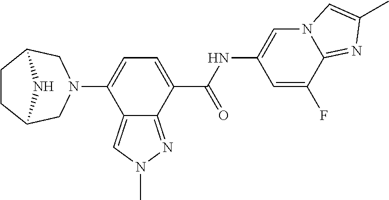 [SMILES: Cc1cn2cc(NC(=O)c3ccc(N4C[C@H]5CC[C@@H](C4)N5)c4cn(C)nc34)cc(F)c2n1] |
| 712 | 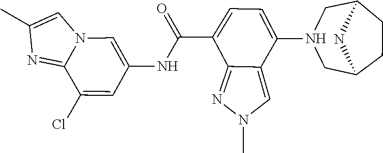 [SMILES: Cc1cn2cc(NC(=O)c3ccc(N4C[C@H]5CC[C@@H](C4)N5)c4cn(C)nc34)cc(Cl)c2n1] |
| 713 | 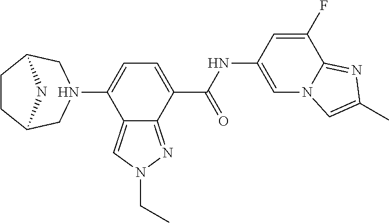 [SMILES: CCn1cc2c(N3C[C@H]4CC[C@@H](C3)N4)ccc(C(=O)Nc3cc(F)c4nc(C)cn4c3)c2n1] |
| 714 | 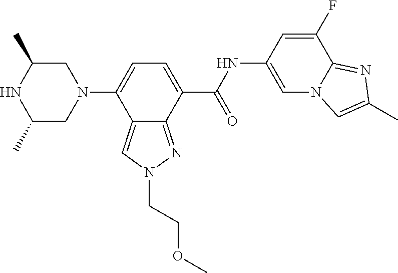 [SMILES: COCCn1cc2c(N3C[C@H](C)N[C@@H](C)C3)ccc(C(=O)Nc3cc(F)c4nc(C)cn4c3)c2n1] |
| 715 | 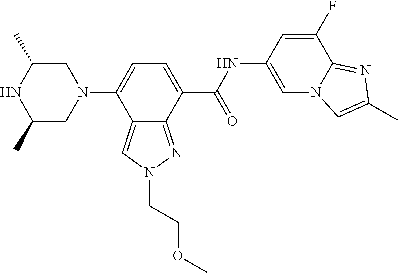 [SMILES: COCCn1cc2c(N3C[C@@H](C)N[C@H](C)C3)ccc(C(=O)Nc3cc(F)c4nc(C)cn4c3)c2n1] |
| 716 | 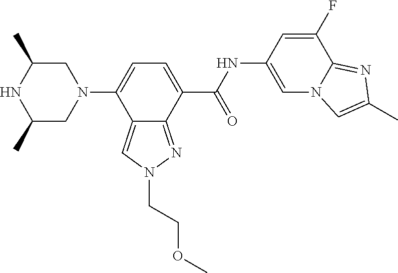 [SMILES: COCCn1cc2c(N3C[C@@H](C)N[C@@H](C)C3)ccc(C(=O)Nc3cc(F)c4nc(C)cn4c3)c2n1] |
| 717 | 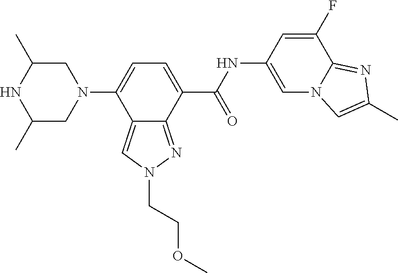 [SMILES: COCCn1cc2c(N3CC(C)NC(C)C3)ccc(C(=O)Nc3cc(F)c4nc(C)cn4c3)c2n1] |
| 718 | 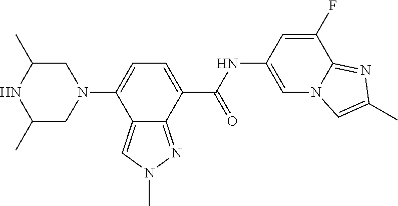 [SMILES: Cc1cn2cc(NC(=O)c3ccc(N4CC(C)NC(C)C4)c4cn(C)nc34)cc(F)c2n1] |
| 719 | 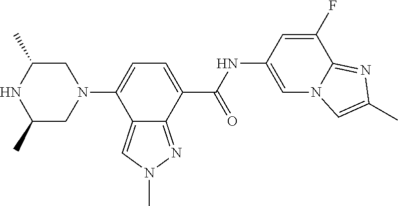 [SMILES: Cc1cn2cc(NC(=O)c3ccc(N4C[C@@H](C)N[C@H](C)C4)c4cn(C)nc34)cc(F)c2n1] |
| 720 | 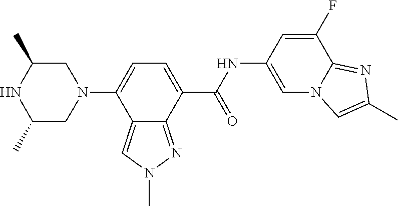 [SMILES: Cc1cn2cc(NC(=O)c3ccc(N4C[C@H](C)N[C@@H](C)C4)c4cn(C)nc34)cc(F)c2n1] |
| 721 | 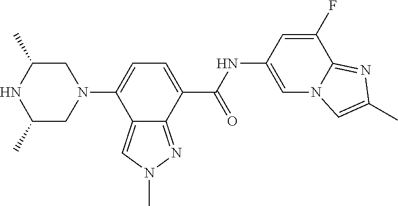 [SMILES: Cc1cn2cc(NC(=O)c3ccc(N4C[C@@H](C)N[C@@H](C)C4)c4cn(C)nc34)cc(F)c2n1] |
| 722 | 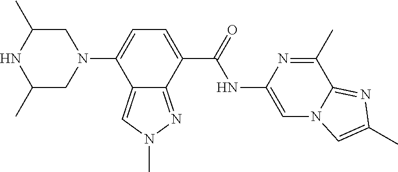 [SMILES: Cc1cn2cc(NC(=O)c3ccc(N4CC(C)NC(C)C4)c4cn(C)nc34)nc(C)c2n1] |
| 723 | 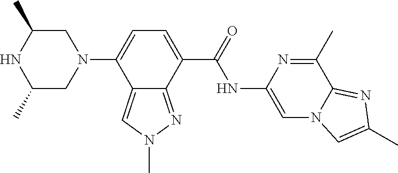 [SMILES: Cc1cn2cc(NC(=O)c3ccc(N4C[C@H](C)N[C@@H](C)C4)c4cn(C)nc34)nc(C)c2n1] |
| 724 | 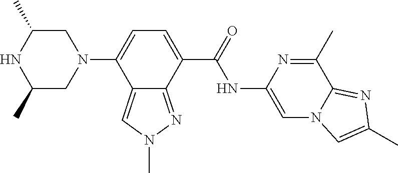 [SMILES: Cc1cn2cc(NC(=O)c3ccc(N4C[C@@H](C)N[C@H](C)C4)c4cn(C)nc34)nc(C)c2n1] |
| 725 | 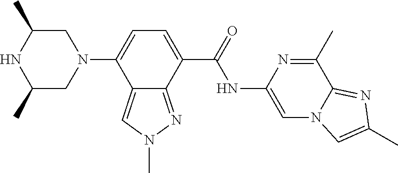 [SMILES: Cc1cn2cc(NC(=O)c3ccc(N4C[C@@H](C)N[C@@H](C)C4)c4cn(C)nc34)nc(C)c2n1] |
| 726 | 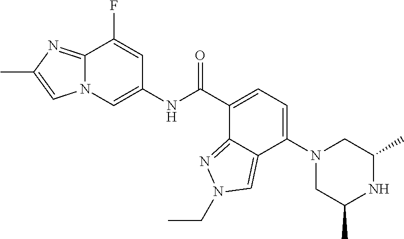 |
| 727 | 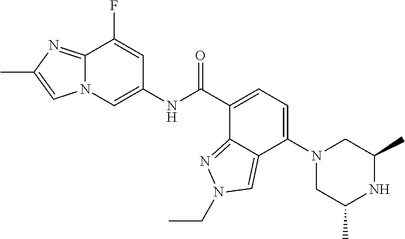 |
| 728 | 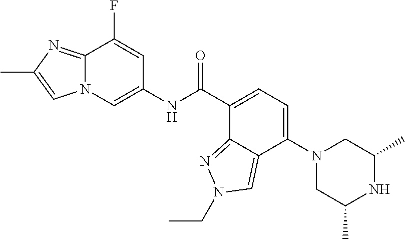 |
| 729 | 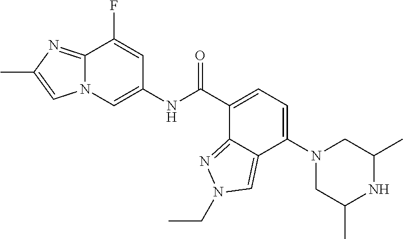 |
| 730 | 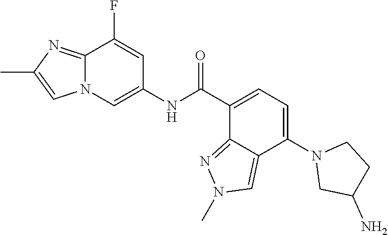 [SMILES: Cc1cn2cc(NC(=O)c3ccc(N4CCC(N)C4)c4cn(C)nc34)cc(F)c2n1] |
| 731 | 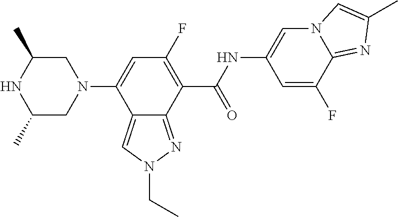 [SMILES: CCn1cc2c(N3C[C@H](C)N[C@@H](C)C3)cc(F)c(C(=O)Nc3cc(F)c4nc(C)cn4c3)c2n1] |
| 732 | 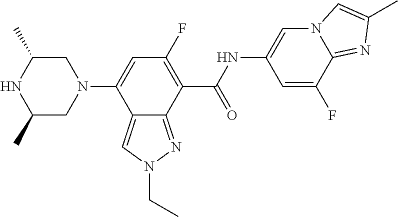 [SMILES: CCn1cc2c(N3C[C@@H](C)N[C@H](C)C3)cc(F)c(C(=O)Nc3cc(F)c4nc(C)cn4c3)c2n1] |
| 733 | 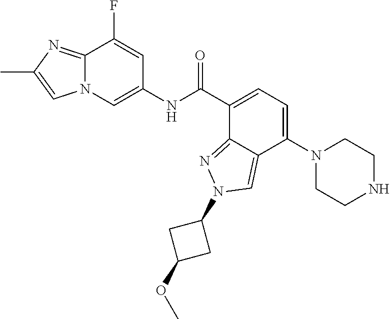 [SMILES: CO[C@H]1C[C@@H](n2cc3c(N4CCNCC4)ccc(C(=O)Nc4cc(F)c5nc(C)cn5c4)c3n2)C1] |
| 734 | 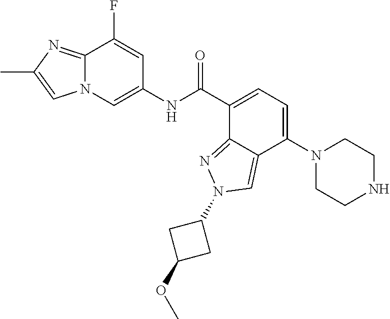 |
| 735 | 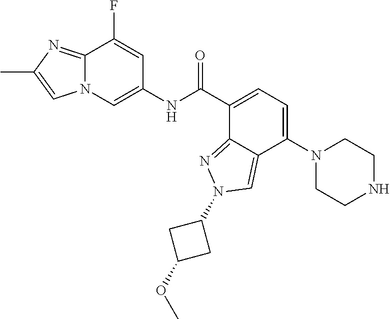 |
| 736 | 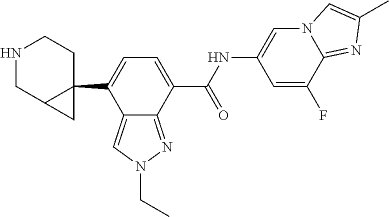 [SMILES: CCn1cc2c([C@]34CCNCC3C4)ccc(C(=O)Nc3cc(F)c4nc(C)cn4c3)c2n1] |
| 737 | 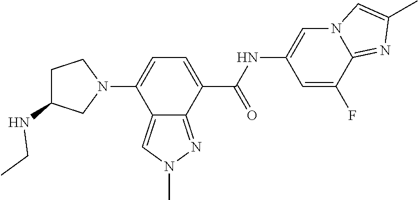 [SMILES: CCN[C@H]1CCN(c2ccc(C(=O)Nc3cc(F)c4nc(C)cn4c3)c3nn(C)cc23)C1] |
| 738 | 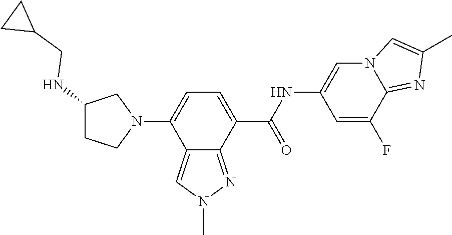 [SMILES: Cc1cn2cc(NC(=O)c3ccc(N4CC[C@H](NCC5CC5)C4)c4cn(C)nc34)cc(F)c2n1] |
| 739 | 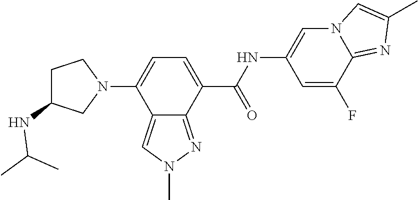 [SMILES: Cc1cn2cc(NC(=O)c3ccc(N4CC[C@H](NC(C)C)C4)c4cn(C)nc34)cc(F)c2n1] |
| 740 | 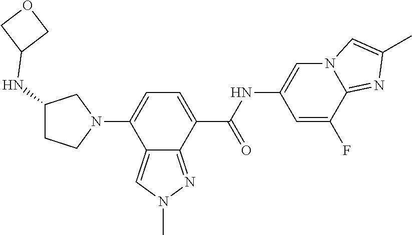 [SMILES: Cc1cn2cc(NC(=O)c3ccc(N4CC[C@H](NC5COC5)C4)c4cn(C)nc34)cc(F)c2n1] |
| 741 | 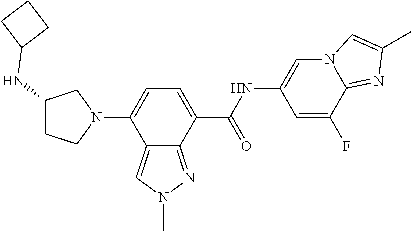 [SMILES: Cc1cn2cc(NC(=O)c3ccc(N4CC[C@H](NC5CCC5)C4)c4cn(C)nc34)cc(F)c2n1] |
| 742 | 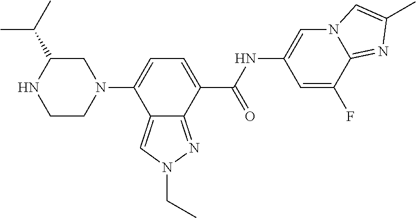 |
| 743 | 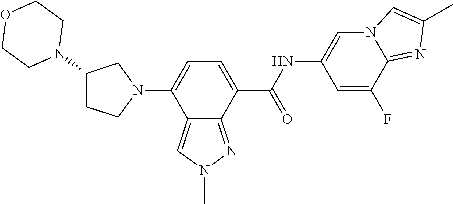 [SMILES: Cc1cn2cc(NC(=O)c3ccc(N4CC[C@H](N5CCOCC5)C4)c4cn(C)nc34)cc(F)c2n1] |
| 744 | 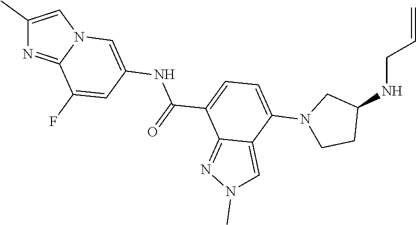 [SMILES: C=CCN[C@H]1CCN(c2ccc(C(=O)Nc3cc(F)c4nc(C)cn4c3)c3nn(C)cc23)C1] |
| 745 | 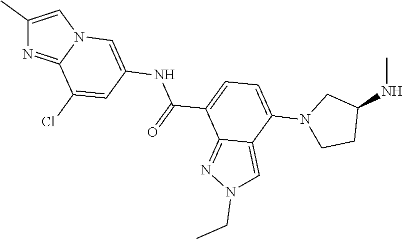 |
| 746 | 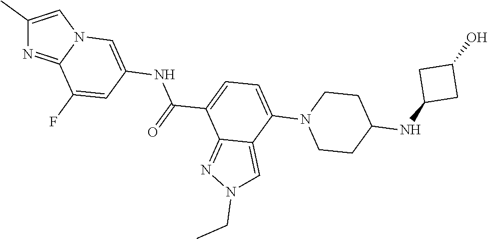 [SMILES: CCn1cc2c(N3CCC(N[C@H]4C[C@H](O)C4)CC3)ccc(C(=O)Nc3cc(F)c4nc(C)cn4c3)c2n1] |
| 747 | 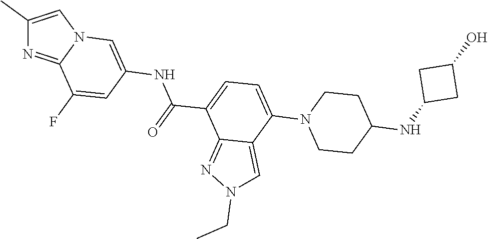 [SMILES: CCn1cc2c(N3CCC(N[C@H]4C[C@@H](O)C4)CC3)ccc(C(=O)Nc3cc(F)c4nc(C)cn4c3)c2n1] |
| 748 | 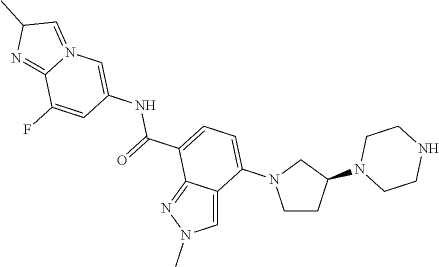 |
| 749 | 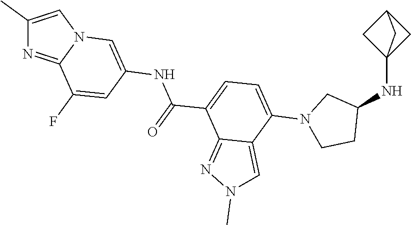 [SMILES: Cc1cn2cc(NC(=O)c3ccc(N4CC[C@H](NC56CC(C5)C6)C4)c4cn(C)nc34)cc(F)c2n1] |
| 750 | 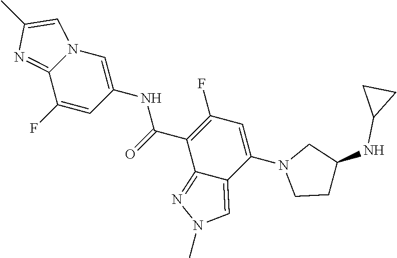 [SMILES: Cc1cn2cc(NC(=O)c3c(F)cc(N4CC[C@H](NC5CC5)C4)c4cn(C)nc34)cc(F)c2n1] |
| 751 | 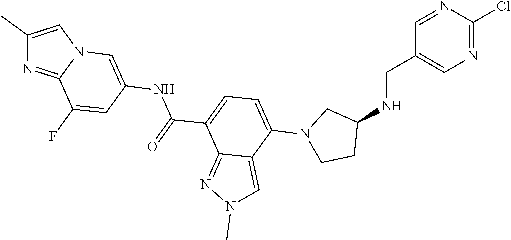 [SMILES: Cc1cn2cc(NC(=O)c3ccc(N4CC[C@H](NCc5cnc(Cl)nc5)C4)c4cn(C)nc34)cc(F)c2n1] |
| 752 | 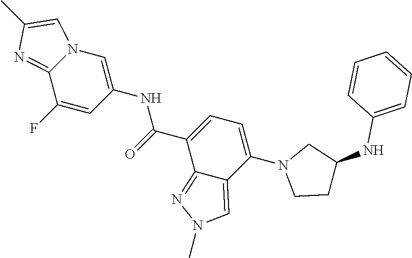 [SMILES: Cc1cn2cc(NC(=O)c3ccc(N4CC[C@H](Nc5ccccc5)C4)c4cn(C)nc34)cc(F)c2n1] |
| 753 | 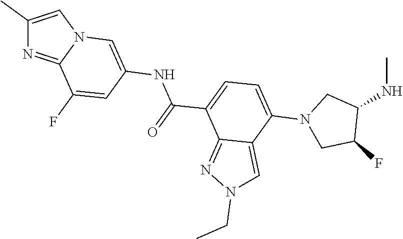 |
| 754 | 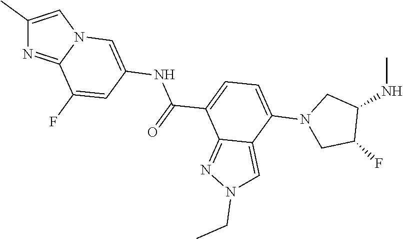 |
| 755 | 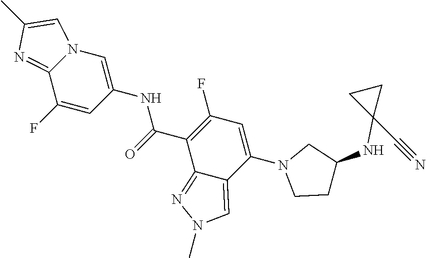 [SMILES: Cc1cn2cc(NC(=O)c3c(F)cc(N4CC[C@H](NC5(C#N)CC5)C4)c4cn(C)nc34)cc(F)c2n1] |
| 756 | 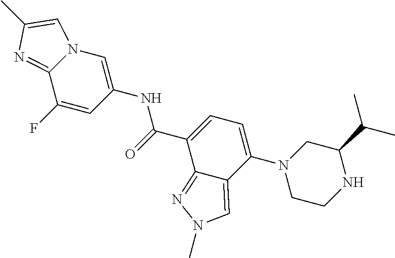 [SMILES: Cc1cn2cc(NC(=O)c3ccc(N4CCN[C@H](C(C)C)C4)c4cn(C)nc34)cc(F)c2n1] |
| 757 | 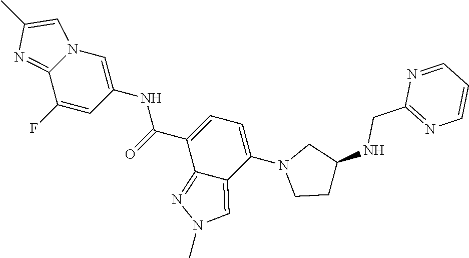 [SMILES: Cc1cn2cc(NC(=O)c3ccc(N4CC[C@H](NCc5ncccn5)C4)c4cn(C)nc34)cc(F)c2n1] |
| 758 | 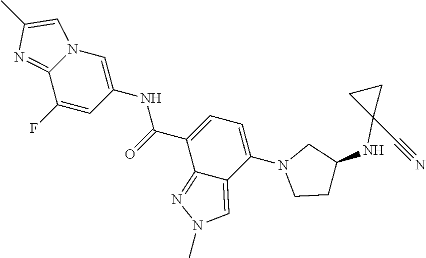 [SMILES: Cc1cn2cc(NC(=O)c3ccc(N4CC[C@H](NC5(C#N)CC5)C4)c4cn(C)nc34)cc(F)c2n1] |
| 759 | 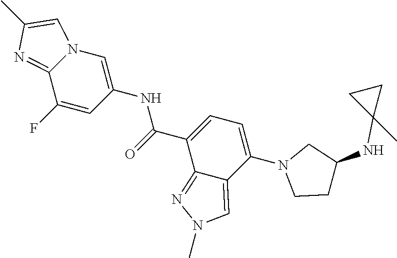 |
| 760 | 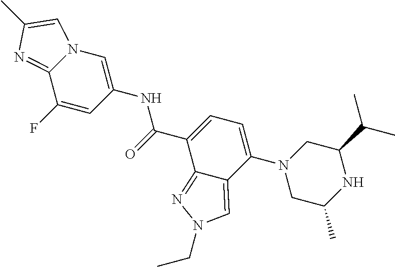 [SMILES: CCn1cc2c(N3C[C@@H](C)N[C@H](C(C)C)C3)ccc(C(=O)Nc3cc(F)c4nc(C)cn4c3)c2n1] |
| 761 | 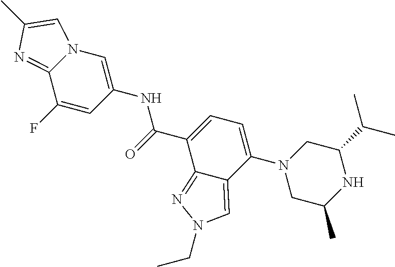 [SMILES: CCn1cc2c(N3C[C@H](C)N[C@@H](C(C)C)C3)ccc(C(=O)Nc3cc(F)c4nc(C)cn4c3)c2n1] |
| 762 | 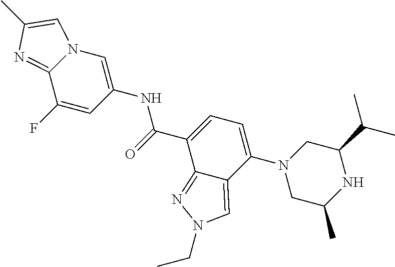 [SMILES: CCn1cc2c(N3C[C@@H](C(C)C)N[C@@H](C)C3)ccc(C(=O)Nc3cc(F)c4nc(C)cn4c3)c2n1] |
| 763 | 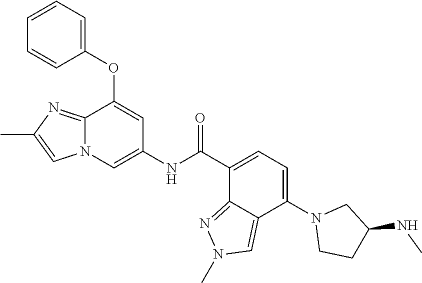 |
| 764 | 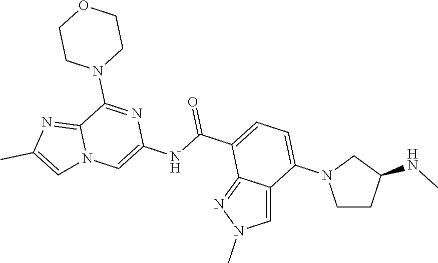 |
| 765 | 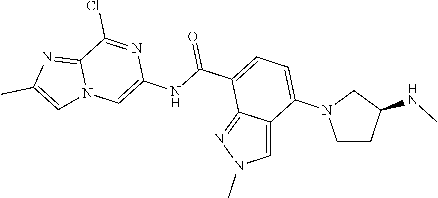 |
| 766 | 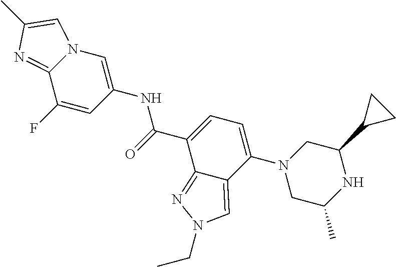 [SMILES: CCn1cc2c(N3C[C@@H](C)N[C@H](C4CC4)C3)ccc(C(=O)Nc3cc(F)c4nc(C)cn4c3)c2n1] |
| 767 | 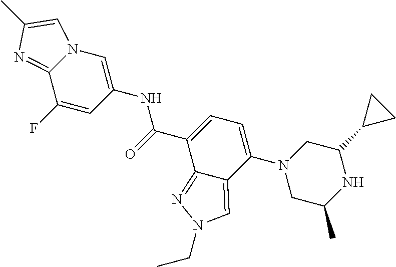 |
| 768 | 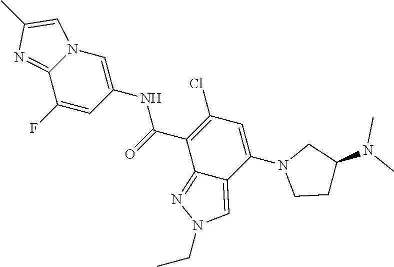 |
| 769 | 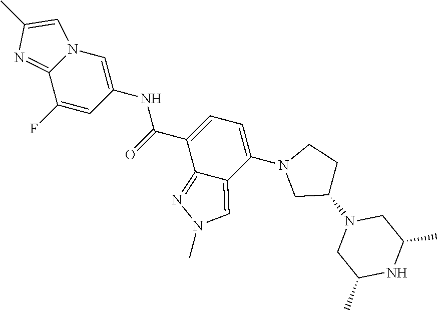 |
| 770 | 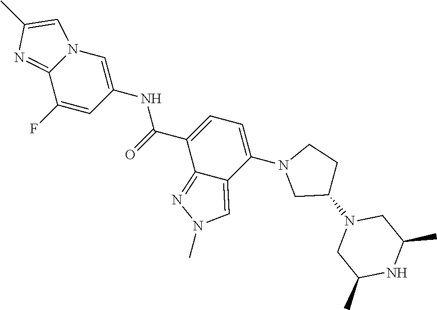 |
| 771 | 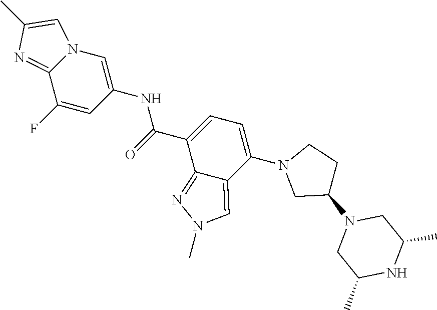 |
| 772 | 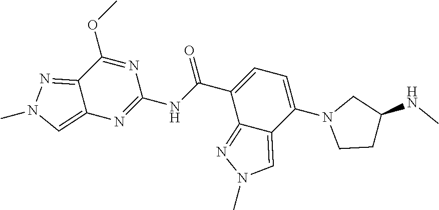 |
| 773 | 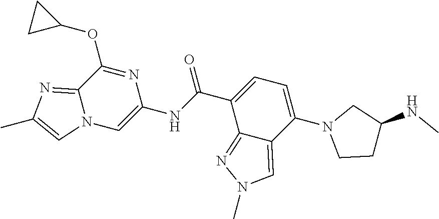 |
| 774 | 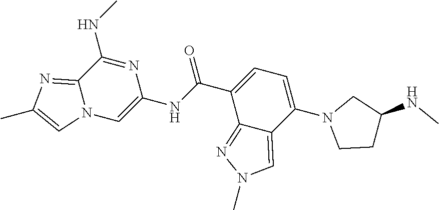 |
| 775 | 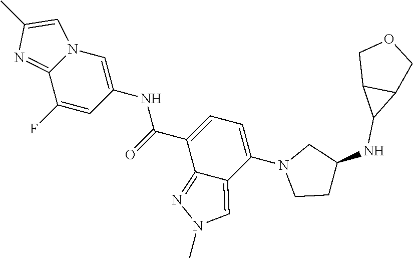 [SMILES: Cc1cn2cc(NC(=O)c3ccc(N4CC[C@H](NC5C6COCC65)C4)c4cn(C)nc34)cc(F)c2n1] |
| 776 | 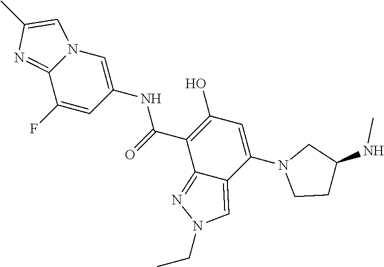 |
| 777 | 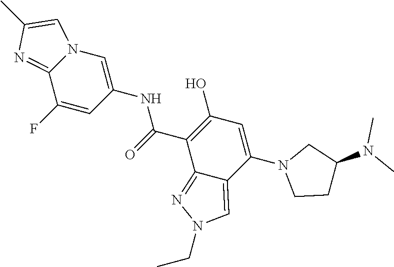 |
| 778 | 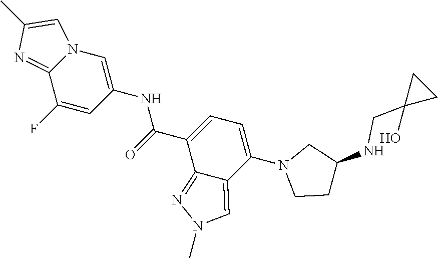 [SMILES: Cc1cn2cc(NC(=O)c3ccc(N4CC[C@H](NCC5(O)CC5)C4)c4cn(C)nc34)cc(F)c2n1] |
| 779 | 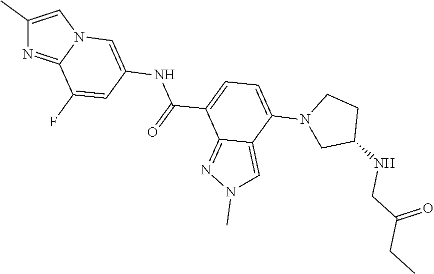 [SMILES: CCC(=O)CN[C@H]1CCN(c2ccc(C(=O)Nc3cc(F)c4nc(C)cn4c3)c3nn(C)cc23)C1] |
| 780 | 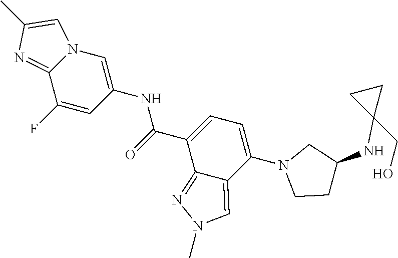 [SMILES: Cc1cn2cc(NC(=O)c3ccc(N4CC[C@H](NC5(CO)CC5)C4)c4cn(C)nc34)cc(F)c2n1] |
| 781 | 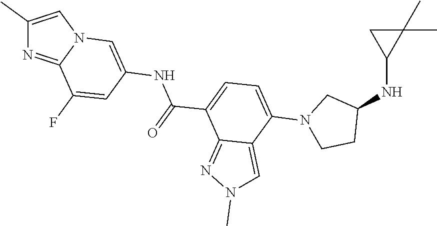 |
| 782 | 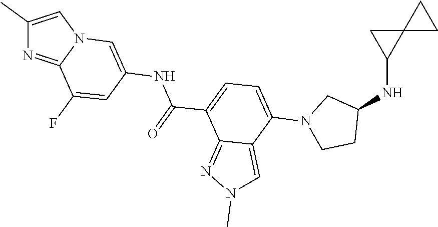 [SMILES: Cc1cn2cc(NC(=O)c3ccc(N4CC[C@H](NC5CC56CC6)C4)c4cn(C)nc34)cc(F)c2n1] |
| 783 | 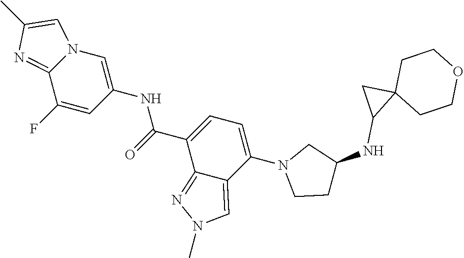 [SMILES: Cc1cn2cc(NC(=O)c3ccc(N4CC[C@H](NC5CC56CCOCC6)C4)c4cn(C)nc34)cc(F)c2n1] |
| 784 | 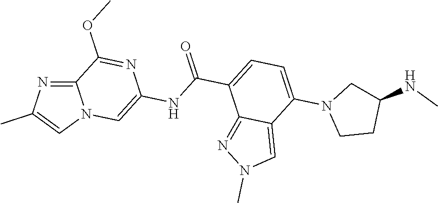 |
| 785 | 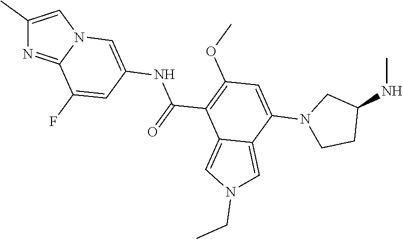 |
| 786 | 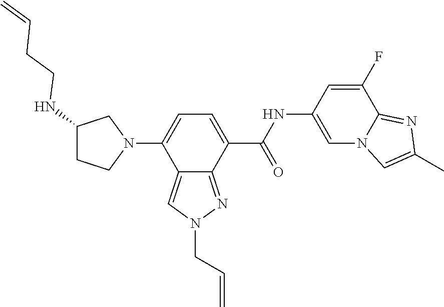 [SMILES: C=CCCN[C@H]1CCN(c2ccc(C(=O)Nc3cc(F)c4nc(C)cn4c3)c3nn(CC=C)cc23)C1] |
| 787 | 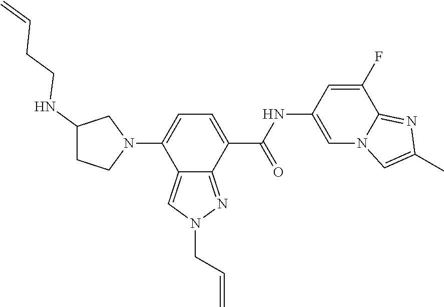 [SMILES: C=CCCNC1CCN(c2ccc(C(=O)Nc3cc(F)c4nc(C)cn4c3)c3nn(CC=C)cc23)C1] |
| 788 | 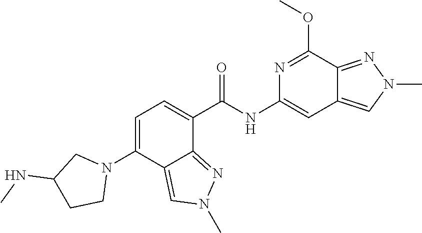 |
| 789 | 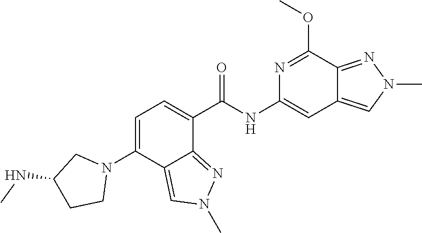 |
| 790 | 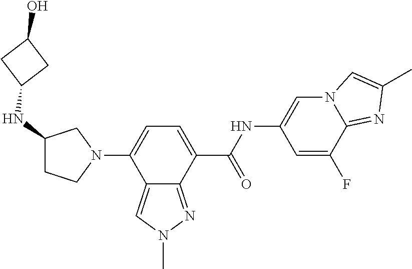 [SMILES: Cc1cn2cc(NC(=O)c3ccc(N4CC[C@@H](N[C@H]5C[C@H](O)C5)C4)c4cn(C)nc34)cc(F)c2n1] |
| 791 | 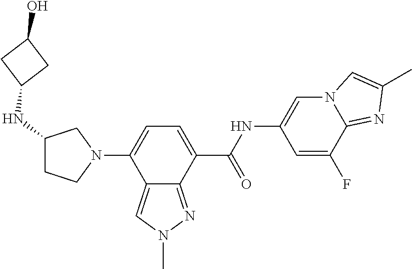 [SMILES: Cc1cn2cc(NC(=O)c3ccc(N4CC[C@H](N[C@H]5C[C@H](O)C5)C4)c4cn(C)nc34)cc(F)c2n1] |
| 792 | 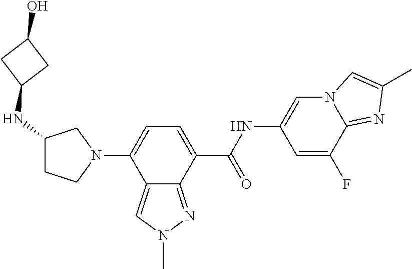 [SMILES: Cc1cn2cc(NC(=O)c3ccc(N4CC[C@H](N[C@H]5C[C@@H](O)C5)C4)c4cn(C)nc34)cc(F)c2n1] |
| 793 | 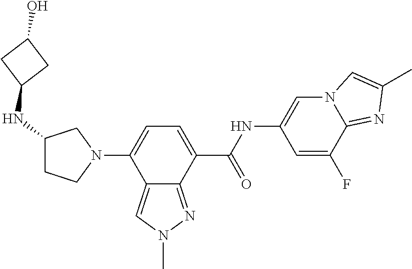 [SMILES: Cc1cn2cc(NC(=O)c3ccc(N4CC[C@H](N[C@H]5C[C@H](O)C5)C4)c4cn(C)nc34)cc(F)c2n1] |
| 794 | 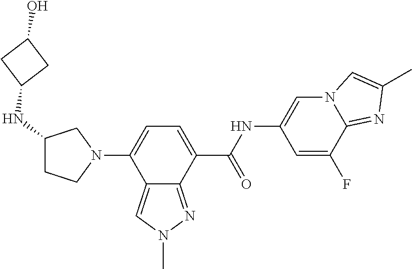 [SMILES: Cc1cn2cc(NC(=O)c3ccc(N4CC[C@H](N[C@H]5C[C@@H](O)C5)C4)c4cn(C)nc34)cc(F)c2n1] |
| 795 | 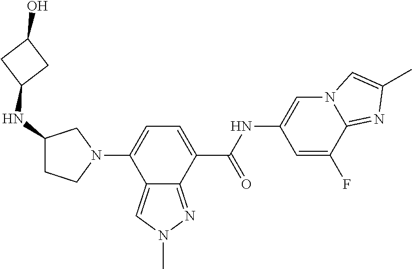 [SMILES: Cc1cn2cc(NC(=O)c3ccc(N4CC[C@@H](N[C@H]5C[C@@H](O)C5)C4)c4cn(C)nc34)cc(F)c2n1] |
| 796 | 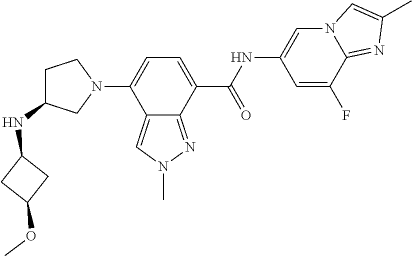 [SMILES: CO[C@H]1C[C@@H](N[C@H]2CCN(c3ccc(C(=O)Nc4cc(F)c5nc(C)cn5c4)c4nn(C)cc34)C2)C1] |
| 797 | 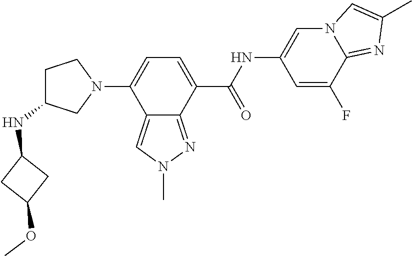 [SMILES: CO[C@H]1C[C@@H](N[C@@H]2CCN(c3ccc(C(=O)Nc4cc(F)c5nc(C)cn5c4)c4nn(C)cc34)C2)C1] |
| 798 | 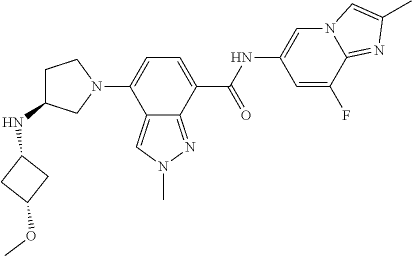 [SMILES: CO[C@H]1C[C@@H](N[C@H]2CCN(c3ccc(C(=O)Nc4cc(F)c5nc(C)cn5c4)c4nn(C)cc34)C2)C1] |
| 799 | 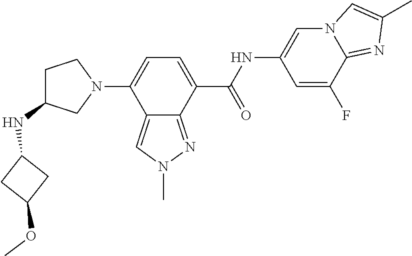 [SMILES: CO[C@H]1C[C@H](N[C@H]2CCN(c3ccc(C(=O)Nc4cc(F)c5nc(C)cn5c4)c4nn(C)cc34)C2)C1] |
| 800 | 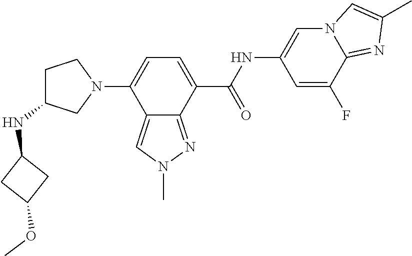 [SMILES: CO[C@H]1C[C@H](N[C@@H]2CCN(c3ccc(C(=O)Nc4cc(F)c5nc(C)cn5c4)c4nn(C)cc34)C2)C1] |
| 801 | 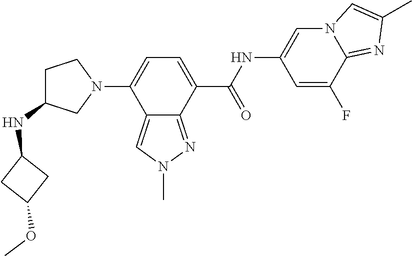 [SMILES: CO[C@H]1C[C@H](N[C@H]2CCN(c3ccc(C(=O)Nc4cc(F)c5nc(C)cn5c4)c4nn(C)cc34)C2)C1] |

In some embodiments, a compound of Formula (I) is selected from Compounds 288-363. In some embodiments, a compound of Formula (I) is a compound other than Compound 118, 119, 140, 141, 142, 143, 145, 146, 147, 148, 149, 150, 187, 188, 189, 190, 191, 192, 193, 194, 195, 196, 197, 198, 199, 200, 201, 202, 203, 204, 205, 206, 207, 208, 209, 210, 211, 212, 217, 218, 219, 228, 229, 230, 231, 234, 235, 237, 238, 239, 240, 241, 242, 243, 244, 245, 246, 247, 249, 250, 251, 252, 253, 255, 256, 257, 258, 259, 260, 261, 262, 263, 264, 265, 266, 267, 268, 269, 270, 271, 272, 273, 274, 275, 277, 278, 279, 280, 281, 282, 283, 284, 285, 286, or 287. In some embodiments, a compound of Formula (I) is selected from 141, 143, 190, 198, 202, 207, 208, 209, 210, 228, 229, 234, 237, 239, 240, 242, 243, 246, 250, 251, 252, 255, 258, 260, 266, 269, 272, 275, 277, 278, 279, 284, 285, 288, 293, 293, 294, 296, 298, 301, 302, 306, 310, 312, 314, 315, 350, and 351.

In some embodiments, a compound of Formula (I) is: a) Compound 288, b) Compound 289, c) Compound 290, d) Compound 291, e) Compound 292, f) Compound 293, g) Compound 294, h) Compound 295, i) Compound 296, j) Compound 297, k) Compound 298, l) Compound 299, m) Compound 300, n) Compound 301, o) Compound 302, p) Compound 303, q) Compound 304, r) Compound 305, s) Compound 306, t) Compound 307, u) Compound 308, v) Compound 309, w) Compound 310, x) Compound 311, y) Compound 312, z) Compound 313, aa) Compound 314, bb) Compound 315, cc) Compound 316, dd) Compound 317, ee) Compound 318, ff) Compound 319, gg) Compound 320, hh) Compound 321, ii) Compound 322, jj) Compound 323, kk) Compound 324, ll) Compound 325, mm) Compound 326, nn) Compound 327, oo) Compound 328, pp) Compound 329, qq) Compound 330, Compound rr) Compound 331, ss) Compound 332, tt) Compound 333, uu) Compound 334, vv) Compound 335, ww) Compound 336, xx) Compound 337, yy) Compound 338, zz) Compound 339, aaa) Compound 340, bbb) Compound 341, ccc) Compound 342, ddd) Compound 343, eee) Compound 344, fff) Compound 345, ggg) Compound 346, hhh) Compound 347, iii) Compound 348, jjj) Compound 349, kkk) Compound 350, 111) Compound 351, mmm) Compound 352, nnn) Compound 353, ooo) Compound 354, ppp) Compound 355, qqq) Compound 356, rrr) Compound 357, sss) Compound 358, ttt) Compound 359, uuu) Compound 360, vvv) Compound 361, www) Compound 362, or xxx) Compound 363.

In some embodiments, for Formula (I), A is monocyclic heterocyclyl (e.g., piperidinyl); B is bicyclic heteroaryl (e.g., imidazo[1,2-b]pyridazinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 118, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., imidazo[1,2-b]pyridazinyl); L² is —C(O)N(R⁴)— (e.g., —C(O)N(H)—); X and Y are each independently C(R^{3a}) (e.g., CH); Z is O; y is 0; and m is 0. In some embodiments, the compound of Formulas (I), (I-a), (I-b), (I-c), (I-e), (I-f), (I-h) and (I-i) is Compound 119, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., imidazo[1,2-b]pyridazinyl); L¹ is absent or —N(R⁴)—; and L² is absent or —C(O)N(R⁴)— (e.g., —C(O)N(H)—). In some embodiments, for Formula (I), the compound is selected from Compound 118, 141, 228, 229, 242, 243, 269, and 277.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is N; Y is C(R^{3a}) (e.g., C(CH₃)); Z is O; y is 0; and m is 0. In some embodiments, the compound of Formulas (I), (I-a), (I-b), (I-e), (I-f), and (I-i) is Compound 140, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is 0; Y is C(R^{3a}) (e.g., C(CH₃)); Z is N; y is 0; and m is 0. In some embodiments, the compound of Formulas (I), (I-a), (I-b), (I-e), (I-f), and (I-i) is Compound 141, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 142, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 143, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 145, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is N(R^{3c}) (e.g., NH); Y is C(R^{3a}) (e.g., C(CH₃)); Z is N; y is 0; and m is 0. In some embodiments, the compound of Formulas (I), (I-a), (I-b), (I-e), (I-f), and (I-i) is Compound 146, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is N(R^{3c}) (e.g., NH); Y is N; Z is N; y is 0; and m is 0. In some embodiments, the compound of Formulas (I), (I-a), (I-b), (I-e), (I-f), and (I-i) is Compound 147, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,8-dimethylimidazo[1,2-b]pyridazinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 148, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., CH); Z is O; y is 0; and m is 0. In some embodiments, the compound of Formulas (I), (I-a), (I-b), (I-c), (I-e), (I-f), (I-h), and (I-i) is Compound 149, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is N; Y is C(R^{3a}) (e.g., CH); Z is O; y is 0; and m is 0. In some embodiments, the compound of Formulas (I), (I-a), (I-b), (I-e), (I-f), and (I-i) is Compound 150, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L² is —C(O)N(R⁴)— (e.g., —C(O)N(H)—); X is 0; Y is C(R^{3a}) (e.g., C(CH₃)); Z is N; y is 0; and m is 0. In some embodiments, the compound of Formulas (I), (I-a), (I-b), (I-e), (I-f), and (I-i) is Compound 187, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a})(R^{3b}) (e.g., CH₂); Y is C(R^{3a})(R^{3b}) (e.g., CH₂); Z is O; y is 0; and m is 0. In some embodiments, the compound of Formulas (I), (I-a), (I-b), (I-e), (I-f), and (I-i) is Compound 188, or a pharmaceutically acceptable salt, solvate, hydrate, tautomer, or stereoisomer thereof.

In some embodiments, for Formula (I), A is monocyclic heterocyclyl (e.g., 1-methylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 189, or a pharmaceutically acceptable salt, solvate, hydrate, tautomer, or stereoisomer thereof.

In some embodiments, for Formula (I), A is monocyclic heterocyclyl (e.g., 2-methylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 190, or a pharmaceutically acceptable salt, solvate, hydrate, tautomer, or stereoisomer thereof.

In some embodiments, for Formula (I), A is monocyclic heterocyclyl (e.g., 1,2-dimethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 191, or a pharmaceutically acceptable salt, solvate, hydrate, tautomer, or stereoisomer thereof.

In some embodiments, for Formula (I), A is monocyclic heterocyclyl (e.g., 2-ethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 192, or a pharmaceutically acceptable salt, solvate, hydrate, tautomer, or stereoisomer thereof.

In some embodiments, for Formula (I), A is monocyclic heterocyclyl (e.g., 2,2-dimethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 193, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 194, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 195, or a pharmaceutically acceptable salt, solvate, hydrate, tautomer, or stereoisomer thereof.

In some embodiments, for Formula (I), A is monocyclic heterocyclyl (e.g., 4-(N-ethyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 196, or a pharmaceutically acceptable salt, solvate, hydrate, tautomer, or stereoisomer thereof.

In some embodiments, for Formula (I), A is monocyclic heterocyclyl (e.g., 4-(N-tert-butyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 197, or a pharmaceutically acceptable salt, solvate, hydrate, tautomer, or stereoisomer thereof.

In some embodiments, for Formula (I), A is monocyclic heterocyclyl (e.g., 4-(N,N-dimethyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 198, or a pharmaceutically acceptable salt, solvate, hydrate, tautomer, or stereoisomer thereof.

In some embodiments, for Formula (I), A is bicyclic heterocyclyl (e.g., octahydropyrrolo[1,2-a]pyrazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 199, or a pharmaceutically acceptable salt, solvate, hydrate, tautomer, or stereoisomer thereof.

In some embodiments, for Formula (I), A is monocyclic heterocyclyl (e.g., 1-methylpiperidinyl); B is bicyclic heteroaryl (e.g., 8-fluoro-2-methylimidazo[1,2-a]pyridinyl); L¹ is —N(R⁴)— (e.g., —N(CH₃)—); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I) and (I-a) is Compound 200, or a pharmaceutically acceptable salt, solvate, hydrate, tautomer, or stereoisomer thereof.

In some embodiments, for Formula (I), A is bicyclic heterocyclyl (e.g., 2,2,6,6-tetramethylpiperidinyl); B is bicyclic heteroaryl (e.g., 8-fluoro-2-methylimidazo[1,2-a]pyridinyl); L¹ is —N(R⁴)— (e.g., —N(CH₃)—); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I) and (I-a) is Compound 201 or a pharmaceutically acceptable salt, solvate, hydrate, tautomer, or stereoisomer thereof.

In some embodiments, for Formula (I), A is monocyclic heterocyclyl (e.g., 3-(N,N-dimethyl)aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 202, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 203, or a pharmaceutically acceptable salt, solvate, hydrate, tautomer, or stereoisomer thereof.

In some embodiments, for Formula (I), A is monocyclic heterocyclyl (e.g., 1,3′-bipyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 204, or a pharmaceutically acceptable salt, solvate, hydrate, tautomer, or stereoisomer thereof.

In some embodiments, for Formula (I), A is bicyclic heterocyclyl (e.g., 2,6-diazaspiro[3.3]heptanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 205, or a pharmaceutically acceptable salt, solvate, hydrate, tautomer, or stereoisomer thereof.

In some embodiments, for Formula (I), A is bicyclic heterocyclyl (e.g 2-methyl-2,6-diazaspiro[3.3]heptanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 206, or a pharmaceutically acceptable salt, solvate, hydrate, tautomer, or stereoisomer thereof.

In some embodiments, for Formula (I), A is monocyclic heterocyclyl (e.g., 1-methylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 207, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 208, or a pharmaceutically acceptable salt, solvate, hydrate, tautomer, or stereoisomer thereof.

In some embodiments, for Formula (I), A is monocyclic heterocyclyl (e.g., 1-methylpiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 209, or a pharmaceutically acceptable salt, solvate, hydrate, tautomer, or stereoisomer thereof.

In some embodiments, for Formula (I), A is monocyclic heterocyclyl (e.g., 1-ethylpiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 210, or a pharmaceutically acceptable salt, solvate, hydrate, tautomer, or stereoisomer thereof.

In some embodiments, for Formula (I), A is monocyclic heterocyclyl (e.g., 2,6-dimethylpiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 211, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 212, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4-fluoro-2-methyl-2H-indazolyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 217, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4-fluoro-2-methylbenzo[d]oxazolyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 218, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4-fluoro-2-methylbenzo[d]thiazolyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 219, or a pharmaceutically acceptable salt, solvate, hydrate, tautomer, or stereoisomer thereof.

In some embodiments, for Formula (I), A is monocyclic heterocyclyl (e.g., 2-methylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 228, 352, 353, or a pharmaceutically acceptable salt, solvate, hydrate, tautomer, or stereoisomer thereof.

In some embodiments, for Formula (I), A is monocyclic heterocyclyl (e.g., 1,2-dimethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 229, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methyl-2H-indazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 230, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4-fluoro-2-methyl-2H-indazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 231, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4-fluoro-2-methylbenzo[d]oxazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 234, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4-fluoro-2-methylbenzo[d]thiazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 235, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,7-dimethylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 237, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,8-dimethylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 238, or a pharmaceutically acceptable salt, solvate, hydrate, tautomer, or stereoisomer thereof.

In some embodiments, for Formula (I), A is monocyclic heterocyclyl (e.g., 2-ethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 239, or a pharmaceutically acceptable salt, solvate, hydrate, tautomer, or stereoisomer thereof.

In some embodiments, for Formula (I), A is monocyclic heterocyclyl (e.g., 2,2-dimethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 240, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 241, or a pharmaceutically acceptable salt, solvate, hydrate, tautomer, or stereoisomer thereof.

In some embodiments, for Formula (I), A is bicyclic heterocyclyl (e.g., 2,6-diazaspiro[3.3]heptanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 242, or a pharmaceutically acceptable salt, solvate, hydrate, tautomer, or stereoisomer thereof.

In some embodiments, for Formula (I), A is bicyclic heterocyclyl (e.g., 2-methyl-2,6-diazaspiro[3.3]heptanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 243, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L¹ is —N(R⁴)— (e.g., —N(CH₃)—); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 244, or a pharmaceutically acceptable salt, solvate, hydrate, tautomer, or stereoisomer thereof.

In some embodiments, for Formula (I), A is monocyclic heterocyclyl (e.g., 2-methylpiperidinyl); B is bicyclic heteroaryl (e.g., 8-fluoro-2-methylimidazo[1,2-a]pyridinyl); L¹ is —N(R⁴)— (e.g., —N(CH₃)—); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 245, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methylimidazo[1,2-a]pyrazinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 246, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4,6-dimethylpyrazolo[1,5-a]pyrazinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 247, or a pharmaceutically acceptable salt, solvate, hydrate, tautomer, or stereoisomer thereof.

In some embodiments, for Formula (I), A is monocyclic heterocyclyl (e.g., 3-(N-tert-butyl)-aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 249, or a pharmaceutically acceptable salt, solvate, hydrate, tautomer, or stereoisomer thereof.

In some embodiments, for Formula (I), A is bicyclic heterocyclyl (e.g., octahydropyrrolo[1,2-a]pyrazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 250, or a pharmaceutically acceptable salt, solvate, hydrate, tautomer, or stereoisomer thereof.

In some embodiments, for Formula (I), A is monocyclic heterocyclyl (e.g., 4-(N,N-dimethyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 251, or a pharmaceutically acceptable salt, solvate, hydrate, tautomer, or stereoisomer thereof.

In some embodiments, for Formula (I), A is monocyclic heterocyclyl (e.g., 4-(N-ethyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 252, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6,8-dimethyl-[1,2,4]triazolo[1,5-a]pyrazinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 253, or a pharmaceutically acceptable salt, solvate, hydrate, tautomer, or stereoisomer thereof.

In some embodiments, for Formula (I), A is bicyclic heterocyclyl (e.g., (1R,5S)-3,8-diazabicyclo[3.2.1]octanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 255, or a pharmaceutically acceptable salt, solvate, hydrate, tautomer, or stereoisomer thereof.

In some embodiments, for Formula (I), A is monocyclic heterocyclyl (e.g., 1-methylpiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 256, or a pharmaceutically acceptable salt, solvate, hydrate, tautomer, or stereoisomer thereof.

In some embodiments, for Formula (I), A is monocyclic heterocyclyl (e.g., 1-ethylpiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 257, or a pharmaceutically acceptable salt, solvate, hydrate, tautomer, or stereoisomer thereof.

In some embodiments, for Formula (I), A is monocyclic heterocyclyl (e.g., 3-(N,N-methyl)aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 258, 350, 351, or a pharmaceutically acceptable salt, solvate, hydrate, tautomer, or stereoisomer thereof.

In some embodiments, for Formula (I), A is monocyclic heterocyclyl (e.g., 4-(N-tert-butyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 259, or a pharmaceutically acceptable salt, solvate, hydrate, tautomer, or stereoisomer thereof.

In some embodiments, for Formula (I), A is bicyclic heterocyclyl (e.g., (1R,5S)-3,8-diazabicyclo[3.2.1]octanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 260, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methyl-2H-indazolyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 261, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,7-dimethylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 262, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methylimidazo[1,2-a]pyrazinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 263, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6,8-dimethyl-[1,2,4]triazolo[1,5-a]pyrazinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-g), and (I-h) is Compound 264, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4,6-dimethylpyrazolo[1,5-a]pyrazinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-g), and (I-h) is Compound 265, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-chloro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 266, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-methyl-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 267, or a pharmaceutically acceptable salt, solvate, hydrate, tautomer, or stereoisomer thereof.

In some embodiments, for Formula (I), A is monocyclic heterocyclyl (e.g., piperazinyl); B is monocyclic heteroaryl (e.g., pyrazolyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), and (I-h) is Compound 268, or a pharmaceutically acceptable salt, solvate, hydrate, tautomer, or stereoisomer thereof.

In some embodiments, for Formula (I), A is bicyclic heterocyclyl (e.g., piperazinyl); B is bicyclic heteroaryl (e.g., 8-chloro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 269, or a pharmaceutically acceptable salt, solvate, hydrate, tautomer, or stereoisomer thereof.

In some embodiments, for Formula (I), A is bicyclic heterocyclyl (e.g., piperazinyl); B is monocyclic heteroaryl (e.g., pyrazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), and (I-e) is Compound 270, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), (I-e), (I-f), (I-h), and (I-i) is Compound 271, or a pharmaceutically acceptable salt, solvate, hydrate, tautomer, or stereoisomer thereof.

In some embodiments, for Formula (I), A is monocyclic heterocyclyl (e.g., 1,3′-bipyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 272, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6,8-dimethylimidazo[1,2-a]pyrazinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 273, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 274, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methyl-8-(trifluoromethyl)imidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 275, or a pharmaceutically acceptable salt, solvate, hydrate, tautomer, or stereoisomer thereof.

In some embodiments, for Formula (I), A is monocyclic heterocyclyl (e.g., 2-methylpiperazinyl); B is bicyclic heteroaryl (e.g., 8-chloro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 277, or a pharmaceutically acceptable salt, solvate, hydrate, tautomer, or stereoisomer thereof.

In some embodiments, for Formula (I), A is monocyclic heterocyclyl (e.g., 3-(N,N-dimethyl)aminopyrrolidinyl); B is bicyclic heteroaryl (e.g., 8-chloro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 278, or a pharmaceutically acceptable salt, solvate, hydrate, tautomer, or stereoisomer thereof.

In some embodiments, for Formula (I), A is bicyclic heterocyclyl (e.g., (1R,5S)-3,8-diazabicyclo[3.2.1]octanyl); B is bicyclic heteroaryl (e.g., 8-chloro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 279, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), and (I-e) is Compound 280, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazinyl); B is monocyclic heterocyclyl (e.g., piperidinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), and (I-e is Compound 281, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazinyl); B is monocyclic heterocyclyl (e.g., piperidinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), and (I-e) is Compound 282, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L² is —C(O)N(R⁴)— (e.g., —C(O)N(H)—); X is C(R^{3a}) (e.g., CH); Y is C(R^{3a}) (e.g., C(CH₃)); Z is O; y is 0; and m is 0. In some embodiments, the compound of Formulas (I), (I-a), (I-b), (I-c), and (I-e) is Compound 283, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-chloro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CH₃)); Z is N; y is 0; and m is 0. In some embodiments, the compound of Formulas (I), (I-a), (I-b), (I-d), (I-e), (I-f), (I-i), and (I-j) is Compound 284, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-chloro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 285, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-chloro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NH); Z is N; y is 0; and m is 0. In some embodiments, the compound of Formulas (I), (I-a), (I-b), (I-d), (I-e), (I-f), (I-i), and (I-j) is Compound 286, or a pharmaceutically acceptable salt, solvate, hydrate, tautomer, or stereoisomer thereof.

In some embodiments, for Formula (I), A is monocyclic heterocyclyl (e.g., 1-methylpiperazinyl); B is bicyclic heteroaryl (e.g., 8-chloro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NH); Z is N; y is 0; and m is 0. In some embodiments, the compound of Formulas (I), (I-a), (I-b), (I-d), (I-e), (I-f), (I-i), and (I-j) is Compound 287, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CH₃)); Z is N; y is 0; and m is 0. In some embodiments, the compound of Formulas (I), (I-a), (I-b), (I-d), (I-e), (I-f), (I-i), and (I-j) is Compound 288, or a pharmaceutically acceptable salt, solvate, hydrate, tautomer, or stereoisomer thereof.

In some embodiments, for Formula (I), A is bicyclic heterocyclyl (e.g., (1R,5S)-3,8-diazabicyclo[3.2.1]octanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CH₃)); Z is N; y is 0; and m is 0. In some embodiments, the compound of Formulas (I), (I-a), (I-b), (I-d), (I-e), (I-f), (I-i), and (I-j) is Compound 289, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH(CH₃)₂)); Z is N; y is 0; and m is 0. In some embodiments, the compound of Formulas (I), (I-a), (I-b), (I-d), (I-e), (I-f), (I-i), and (I-j) is Compound 290, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CH₂OH)); Z is N; y is 0; and m is 0. In some embodiments, the compound of Formulas (I), (I-a), (I-b), (I-d), (I-e), (I-f), (I-i), and (I-j) is Compound 291, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CH₂OCH₃)); Z is N; y is 0; and m is 0. In some embodiments, the compound of Formulas (I), (I-a), (I-b), (I-d), (I-e), (I-f), (I-i), and (I-j) is Compound 292, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,8-dimethylimidazo[1,2-a]pyrazinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 293, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) wherein R³, is C₁-C₆-cycloalkyl (e.g., cyclopropyl); Z is N; y is 0; and m is 0. In some embodiments, the compound of Formulas (I), (I-a), (I-b), (I-d), (I-e), (I-f), (I-i), and (I-j) is Compound 294, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) wherein R^{3c} is heterocyclyl (e.g., oxetane); Z is N; y is 0; and m is 0. In some embodiments, the compound of Formulas (I), (I-a), (I-b), (I-d), (I-e), (I-f), (I-i), and (I-j) is Compound 295, or a pharmaceutically acceptable salt, solvate, hydrate, tautomer, or stereoisomer thereof.

In some embodiments, for Formula (I), A is monocyclic heterocyclyl (e.g., 3-(N-cyclopropyl)aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 296, 371, 372, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., C(CH₃)); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 297, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 298, or a pharmaceutically acceptable salt, solvate, hydrate, tautomer, or stereoisomer thereof.

In some embodiments, for Formula (I), A is bicyclic heterocyclyl (e.g., 1,6-diazaspiro[3.4]octanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 299, or a pharmaceutically acceptable salt, solvate, hydrate, tautomer, or stereoisomer thereof.

In some embodiments, for Formula (I), A is bicyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; R² is C₁-C₆ alkyl (e.g., CH₃); y is 0; and m is 1. In some embodiments, the compound of Formulas (I), (I-a), (I-b), (I-d), (I-e), (I-f), (I-i), and (I-j) is Compound 300, or a pharmaceutically acceptable salt, solvate, hydrate, tautomer, or stereoisomer thereof.

In some embodiments, for Formula (I), A is bicyclic heterocyclyl (e.g., 1,7-diazaspiro[3.5]nonanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 301, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CH₂CH₂OCH₃)); Z is N; y is 0; and m is 0. In some embodiments, the compound of Formulas (I), (I-a), (I-b), (I-d), (I-e), (I-f), (I-i), and (I-j) is Compound 302, or a pharmaceutically acceptable salt, solvate, hydrate, tautomer, or stereoisomer thereof.

In some embodiments, for Formula (I), A is bicyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; R² is halo (e.g., F); y is 0; and m is 1. In some embodiments, the compound of Formulas (I), (I-a), (I-b), (I-d), (I-e), (I-f), (I-i), and (I-j) is Compound 303, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R^{3c} is tetrahydro-2H-pyranyl; Z is N; y is 0; and m is 0. In some embodiments, the compound of Formulas (I), (I-a), (I-b), (I-d), (I-e), (I-f), (I-i), and (I-j) is Compound 304, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g, NCH₂CH₂F); Z is N; y is 0; and m is 0. In some embodiments, the compound of Formulas (I), (I-a), (I-b), (I-d), (I-e), (I-f), (I-i), and (I-j) is Compound 305, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g, NCH₂CH₂OCH₃); Z is N; y is 0; and m is 0. In some embodiments, the compound of Formulas (I), (I-a), (I-b), (I-d), (I-e), (I-f), (I-i), and (I-j) is Compound 306, or a pharmaceutically acceptable salt, solvate, hydrate, tautomer, or stereoisomer thereof.

In some embodiments, for Formula (I), A is monocyclic heterocyclyl (e.g., 4-(N-ethyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g, NCH₂CH₂OCH₃); Z is N; y is 0; and m is 0. In some embodiments, the compound of Formulas (I), (I-a), (I-b), (I-d), (I-e), (I-f), (I-i), and (I-j) is Compound 307, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆ alkyl substituted with R⁸; R⁸ is heterocyclyl (e.g., oxetanyl); Z is N; y is 0; and m is 0. In some embodiments, the compound of Formulas (I), (I-a), (I-b), (I-d), (I-e), (I-f), (I-i), and (I-j) is Compound 308, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g, NCH₂C(O)N(CH₃)₂); Z is N; y is 0; and m is 0. In some embodiments, the compound of Formulas (I), (I-a), (I-b), (I-d), (I-e), (I-f), (I-i), and (I-j) is Compound 309, or a pharmaceutically acceptable salt, solvate, hydrate, tautomer, or stereoisomer thereof.

In some embodiments, for Formula (I), A is monocyclic heterocyclyl (e.g., 3-(N-ethyl)aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g, NCH₂CH₂OCH₃); Z is N; y is 0; and m is 0. In some embodiments, the compound of Formulas (I), (I-a), (I-b), (I-d), (I-e), (I-f), (I-i), and (I-j) is Compound 310, or a pharmaceutically acceptable salt, solvate, hydrate, tautomer, or stereoisomer thereof.

In some embodiments, for Formula (I), A is bicyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; R² is —OR^{A} (e.g., OCH₃); y is 0; and m is 1. In some embodiments, the compound of Formulas (I), (I-a), (I-b), (I-d), (I-e), (I-f), (I-i), and (I-j) is Compound 311, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g, NCH₂CH₂CH₂CH₃); Z is N; y is 0; and m is 0. In some embodiments, the compound of Formulas (I), (I-a), (I-b), (I-d), (I-e), (I-f), (I-i), and (I-j) is Compound 312, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆-alkyl substituted with R⁸; R⁸ is heteroaryl (e.g, pyridyl); Z is N; y is 0; and m is 0. In some embodiments, the compound of Formulas (I), (I-a), (I-b), (I-d), (I-e), (I-f), (I-i), and (I-j) is Compound 313, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-cyano-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 314, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 7-fluoro-6-methoxy-2-methyl-2H-indazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 315, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CF₃)); Z is N; y is 0; and m is 0. In some embodiments, the compound of Formulas (I), (I-a), (I-b), (I-d), (I-e), (I-f), (I-i), and (I-j) is Compound 316, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆ alkyl substituted with R⁸; R⁸ is heterocyclyl (e.g., oxiranyl); Z is N; y is 0; and m is 0. In some embodiments, the compound of Formulas (I), (I-a), (I-b), (I-d), (I-e), (I-f), (I-i), and (I-j) is Compound 317, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆ alkyl substituted with R⁸; R⁸ is heteroaryl (e.g., pyrimidyl); Z is N; y is 0; and m is 0. In some embodiments, the compound of Formulas (I), (I-a), (I-b), (I-d), (I-e), (I-f), (I-i), and (I-j) is Compound 318, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 7-fluoro-2-methyl-2H-indazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 319, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,7-dimethyl-2H-indazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 320, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 1,2,4-trimethyl-1H-benzo[d]imidazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 321, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,8-dimethylimidazo[1,2-b]pyridazinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 322, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-methoxy-2-methylimidazo[1,2-a]pyrazinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 323, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methyl-2H-indazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 324, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 325, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 326, or a pharmaceutically acceptable salt, solvate, hydrate, tautomer, or stereoisomer thereof.

In some embodiments, for Formula (I), A is monocyclic heterocyclyl (e.g., 3-methanaminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 327, or a pharmaceutically acceptable salt, solvate, hydrate, tautomer, or stereoisomer thereof.

In some embodiments, for Formula (I), A is monocyclic heterocyclyl (e.g., 4-(N-acyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 328, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CH(OH)CH₂(OH))); Z is N; y is 0; and m is 0. In some embodiments, the compound of Formulas (I), (I-a), (I-b), (I-d), (I-e), (I-f), (I-i), and (I-j) is Compound 329, or a pharmaceutically acceptable salt, solvate, hydrate, tautomer, or stereoisomer thereof.

In some embodiments, for Formula (I), A is monocyclic heterocyclyl (e.g., 3-(N,N-diethyl)aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CH₂OCH₃)); Z is N; y is 0; and m is 0. In some embodiments, the compound of Formulas (I), (I-a), (I-b), (I-d), (I-e), (I-f), (I-i), and (I-j) is Compound 330, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂COOH)); Z is N; y is 0; and m is 0. In some embodiments, the compound of Formulas (I), (I-a), (I-b), (I-d), (I-e), (I-f), (I-i), and (I-j) is Compound 331, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4-fluoro-1,2-dimethyl-1H-benzo[d]imidazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 332, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 7-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 333, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 2,8-dimethylimidazo[1,2-a]pyrazinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 334, or a pharmaceutically acceptable salt, solvate, hydrate, tautomer, or stereoisomer thereof.

In some embodiments, for Formula (I), A is monocyclic heterocyclyl (e.g., 4-methanaminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 335, or a pharmaceutically acceptable salt, solvate, hydrate, tautomer, or stereoisomer thereof.

In some embodiments, for Formula (I), A is monocyclic heterocyclyl (e.g., 4-methyl-4 aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 336, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CH₂Cl)); Z is N; y is 0; and m is 0. In some embodiments, the compound of Formulas (I), (I-a), (I-b), (I-d), (I-e), (I-f), (I-i), and (I-j) is Compound 337, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆ alkyl substituted with R⁸; R⁸ is heterocyclyl (e.g., oxetanyl); Z is N; y is 0; and m is 0. In some embodiments, the compound of Formulas (I), (I-a), (I-b), (I-d), (I-e), (I-f), (I-i), and (I-j) is Compound 338, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂C(O)CH₃)); Z is N; y is 0; and m is 0. In some embodiments, the compound of Formulas (I), (I-a), (I-b), (I-d), (I-e), (I-f), (I-i), and (I-j) is Compound 339, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆ alkyl substituted with R⁸; R⁸ is heteroaryl (e.g., 1H-indazolyl); Z is N; y is 0; and m is 0. In some embodiments, the compound of Formulas (I), (I-a), (I-b), (I-d), (I-e), (I-f), (I-i), and (I-j) is Compound 340, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆ alkyl substituted with R⁸; R⁸ is heteroaryl (e.g., pyrazolyl); Z is N; y is 0; and m is 0. In some embodiments, the compound of Formulas (I), (I-a), (I-b), (I-d), (I-e), (I-f), (I-i), and (I-j) is Compound 341, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methylimidazo[1,2-a]pyrazin-8(7H)—I); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 342, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-7-methoxy-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 343, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-methoxy-2,7-dimethyl-2H-indazolyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 344, or a pharmaceutically acceptable salt, solvate, hydrate, tautomer, or stereoisomer thereof.

In some embodiments, for Formula (I), A is monocyclic heterocyclyl (e.g., 4-(N-cyclopropyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 345, or a pharmaceutically acceptable salt, solvate, hydrate, tautomer, or stereoisomer thereof.

In some embodiments, for Formula (I), A is monocyclic heterocyclyl (e.g., 4-fluoro-3-(N-methylamino)pyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 346, or a pharmaceutically acceptable salt, solvate, hydrate, tautomer, or stereoisomer thereof.

In some embodiments, for Formula (I), A is monocyclic heterocyclyl (e.g., piperidinyl) substituted with —N(R^{B})(R^{C}) (e.g., —NH(CH₂-py)); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 347, or a pharmaceutically acceptable salt, solvate, hydrate, tautomer, or stereoisomer thereof.

In some embodiments, for Formula (I), A is bicyclic heterocyclyl (e.g., 2,7-diazaspiro[3.5]nonanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 348, or a pharmaceutically acceptable salt, solvate, hydrate, tautomer, or stereoisomer thereof.

In some embodiments, for Formula (I), A is bicyclic heterocyclyl (e.g., 1,8-diazaspiro[4.5]decanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃)); Z is N; y is 0; and m is 0. In some embodiments, the compound of Formulas (I), (I-a), (I-b), (I-d), (I-e), (I-f), (I-i), and (I-j) is Compound 349, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂C(CH₃)₂(OH))); Z is N; y is 0; and m is 0. In some embodiments, the compound of Formulas (I), (I-a), (I-b), (I-d), (I-e), (I-f), (I-i), and (I-j) is Compound 354, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is cycloalkyl (e.g., cyclobutyl) substituted with R¹; R⁸ is OR^{A} (e.g., OH); Z is N; y is 0; and m is 0. In some embodiments, the compound of Formulas (I), (I-a), (I-b), (I-d), (I-e), (I-f), (I-i), and (I-j) is Compound 355, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R^{3c} is heterocyclyl (e.g., tetrahydrofuranyl); Z is N; y is 0; and m is 0. In some embodiments, the compound of Formulas (I), (I-a), (I-b), (I-d), (I-e), (I-f), (I-i), and (I-j) is Compound 356, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₂CH(OH)CH₃); Z is N; y is 0; and m is 0. In some embodiments, the compound of Formulas (I), (I-a), (I-b), (I-d), (I-e), (I-f), (I-i), and (I-j) is Compound 357, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆ alkyl substituted with R⁸; one R⁸ is cycloalkyl (e.g., cyclobutyl) and one R⁸ is —OR^{A} (e.g., —OH); Z is N; y is 0; and m is 0. In some embodiments, the compound of Formulas (I), (I-a), (I-b), (I-d), (I-e), (I-f), (I-i), and (I-j) is Compound 358, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-hydroxy-2-methylimidazo[1,2-a]pyrazinyl); L² is —C(O)N(R⁴)— (e.g., —C(O)N(H)—); X is C(R^{3a}) (e.g., CH); Y is N(R^{3c}) (e.g., NCH₃); Z is N; y is 0; and m is 0. In some embodiments, the compound of Formulas (I), (I-a), (I-b), (I-d), (I-e), (I-f), (I-i), and (I-j) is Compound 359, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₃); Z is N; R² is OR^{A} (e.g., OH); y is 0; and m is 1. In some embodiments, the compound of Formulas (I), (I-a), (I-b), (I-d), (I-e), (I-f), (I-i), and (I-j) is Compound 360, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₃); Z is N; R² is halo (e.g., F); y is 0; and m is 1. In some embodiments, the compound of Formulas (I), (I-a), (I-b), (I-d), (I-e), (I-f), (I-i), and (I-j) is Compound 361, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₃); Z is N; R² is —N(R^{B})(R^{C}) (e.g., —NH(CH₃)); y is 0; and m is 1. In some embodiments, the compound of Formulas (I), (I-a), (I-b), (I-d), (I-e), (I-f), (I-i), and (I-j) is Compound 362, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆ alkyl (e.g., vinyl); Z is N; y is 0; and m is 0. In some embodiments, the compound of Formulas (I), (I-a), (I-b), (I-d), (I-e), (I-f), (I-i), and (I-j) is Compound 363, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃CF₂CH₃); Z is N; y is 0; and m is 0. In some embodiments, the compound of Formulas (I), (I-a), (I-b), (I-d), (I-e), (I-f), (I-i), and (I-j) is Compound 364, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R^{3c} is cycloalkyl (e.g., cyclobutyl) substituted with R⁸; R⁸ is heterocyclyl (e.g., oxetanyl); Z is N; y is 0; and m is 0. In some embodiments, the compound of Formulas (I), (I-a), (I-b), (I-d), (I-e), (I-f), (I-i), and (I-j) is Compound 365, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆ alkyl substituted with R⁸; R⁸ is heteroaryl (e.g., oxazolyl); Z is N; y is 0; and m is 0. In some embodiments, the compound of Formulas (I), (I-a), (I-b), (I-d), (I-e), (I-f), (I-i), and (I-j) is Compound 366, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₂CH(CH₃)OCH₃); Z is N; y is 0; and m is 0. In some embodiments, the compound of Formulas (I), (I-a), (I-b), (I-d), (I-e), (I-f), (I-i), and (I-j) is Compound 367, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}); R³, is C₁-C₆ alkyl substituted with R⁸; R⁸ is heteroaryl (e.g., 1H-1,2,3-triazolyl) substituted with C₁-C₆ alkyl; Z is N; y is 0; and m is 0. In some embodiments, the compound of Formulas (I), (I-a), (I-b), (I-d), (I-e), (I-f), (I-i), and (I-j) is Compound 368, or a pharmaceutically acceptable salt, solvate, hydrate, tautomer, or stereoisomer thereof.

In some embodiments, for Formula (I), A is monocyclic heterocyclyl (e.g., piperidinyl) substituted with —NR^{B}R^{C} (e.g., —NHCH₂CH₂OH); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃); Z is N; y is 0; and m is 0. In some embodiments, the compound of Formulas (I), (I-a), (I-b), (I-d), (I-e), (I-f), (I-i), and (I-j) is Compound 369, or a pharmaceutically acceptable salt, solvate, hydrate, tautomer, or stereoisomer thereof.

In some embodiments, for Formula (I), A is monocyclic heterocyclyl (e.g., piperidinyl) substituted with —NR^{B}R^{C} (e.g., —NH₂) and haloalkyl (e.g., —CF₃); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃); Z is N; y is 0; and m is 0. In some embodiments, the compound of Formulas (I), (I-a), (I-b), (I-d), (I-e), (I-f), (I-i), and (I-j) is Compound 370, or a pharmaceutically acceptable salt, solvate, hydrate, tautomer, or stereoisomer thereof.

In some embodiments, for Formula (I), A is monocyclic heterocyclyl (e.g., pyrrolidinyl) substituted with —NR^{B}R^{C} (e.g., —NHC(CH₂CH₂)CH₂F); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., N(CH₃); Z is N; y is 0; and m is 0. In some embodiments, the compound of Formulas (I), (I-a), (I-b), (I-d), (I-e), (I-f), (I-i), and (I-j) is Compound 373, or a pharmaceutically acceptable salt, solvate, hydrate, tautomer, or stereoisomer thereof.

In some embodiments, for Formula (I), A is monocyclic heterocyclyl (e.g., 3-(N-methyl)aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₂CH₃); Z is N; y is 0; and m is 0. In some embodiments, the compound of Formulas (I), (I-a), (I-b), (I-d), (I-e), (I-f), (I-i), and (I-j) is Compound 374, or a pharmaceutically acceptable salt, solvate, hydrate, tautomer, or stereoisomer thereof.

In some embodiments, for Formula (I), A is monocyclic heterocyclyl (e.g., 3-(N,N-dimethyl)aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₂CH₃); Z is N; y is 0; and m is 0. In some embodiments, the compound of Formulas (I), (I-a), (I-b), (I-d), (I-e), (I-f), (I-i), and (I-j) is Compound 375, or a pharmaceutically acceptable salt, solvate, hydrate, tautomer, or stereoisomer thereof.

In some embodiments, for Formula (I), A is monocyclic heterocyclyl (e.g., 4-(N-cyclopropyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₂CH₃); Z is N; y is 0; and m is 0. In some embodiments, the compound of Formulas (I), (I-a), (I-b), (I-d), (I-e), (I-f), (I-i), and (I-j) is Compound 376, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₂CH₃); Z is N; y is 0; and m is 0. In some embodiments, the compound of Formulas (I), (I-a), (I-b), (I-d), (I-e), (I-f), (I-i), and (I-j) is Compound 377, or a pharmaceutically acceptable salt, solvate, hydrate, tautomer, or stereoisomer thereof.

In some embodiments, for Formula (I), A is monocyclic heterocyclyl (e.g., 1-methylpiperazinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₂CH₃); Z is N; y is 0; and m is 0. In some embodiments, the compound of Formulas (I), (I-a), (I-b), (I-d), (I-e), (I-f), (I-i), and (I-j) is Compound 378, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₂CH₃); Z is N; y is 0; and m is 0. In some embodiments, the compound of Formulas (I), (I-a), (I-b), (I-d), (I-e), (I-f), (I-i), and (I-j) is Compound 379, or a pharmaceutically acceptable salt, solvate, hydrate, tautomer, or stereoisomer thereof.

In some embodiments, for Formula (I), A is monocyclic heterocyclyl (e.g., 4-(N-ethyl)aminopiperidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₂CH₃); Z is N; y is 0; and m is 0. In some embodiments, the compound of Formulas (I), (I-a), (I-b), (I-d), (I-e), (I-f), (I-i), and (I-j) is Compound 378, or a pharmaceutically acceptable salt, solvate, hydrate, tautomer, or stereoisomer thereof.

In some embodiments, for Formula (I), A is monocyclic heterocyclyl (e.g., 3-aminopyrrolidinyl); B is bicyclic heteroaryl (e.g., 8-fluoro-2-methylimidazo[1,2-a]pyridinyl); L² is —C(O)N(R⁴)— (e.g., —C(O)N(H)—); X is C(R^{3a}) (e.g., CH); Y is N(R^{3c}) (e.g., NCH₃); Z is N; y is 0; and m is 0. In some embodiments, the compound of Formulas (I), (I-a), (I-b), (I-d), (I-e), (I-f), (I-i), and (I-j) is Compound 381, 382, or a pharmaceutically acceptable salt, solvate, hydrate, tautomer, or stereoisomer thereof.

The present disclosure further features compounds of Formula (II). In some embodiments, the compound of Formula (II) is a compound of Formula (II-a):

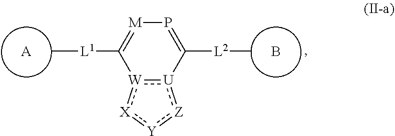

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R⁸; M and P are each independently C(R²) or N; U and W are each independently C or N; X, Y, and Z are each independently C(R^{3a}), N, N(R^{3c}) or S, wherein at least one of X, Y, and Z is N or N(R^{3c}), and the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments of Formula (II),

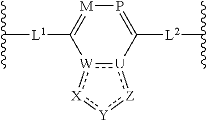

is selected from

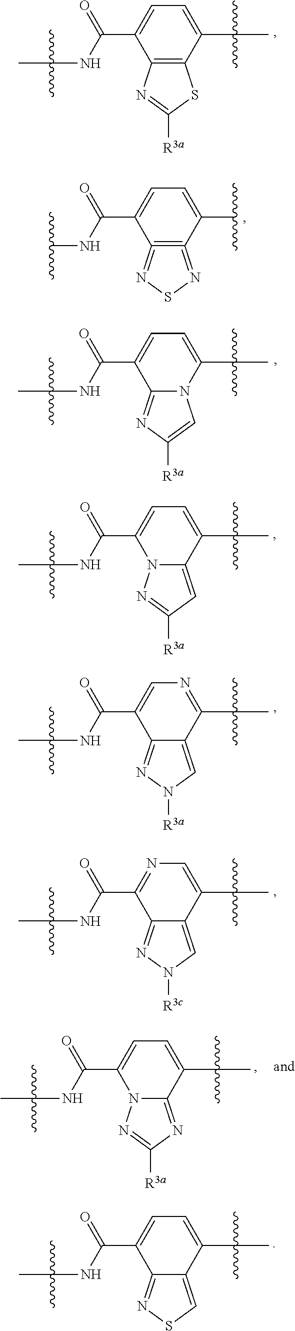

In some embodiments,

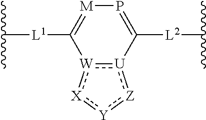

is

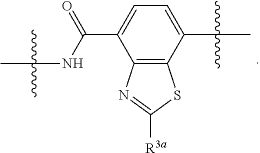

In some embodiments,

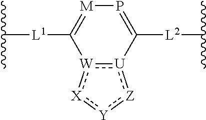

is

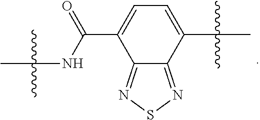 [SMILES: *NC(=O)c1ccc(*)c2nsnc12]

In some embodiments,

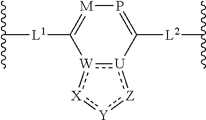

is

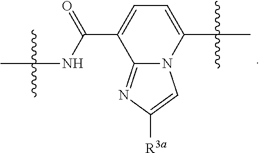

In some embodiments,

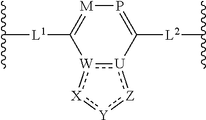

is

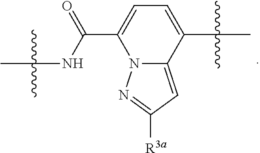

In some embodiments,

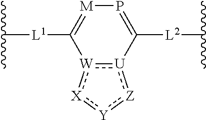

is

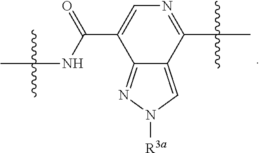

In some embodiments,

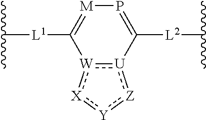

is

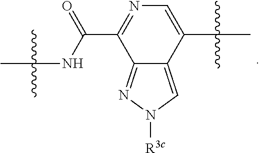

In some embodiments,

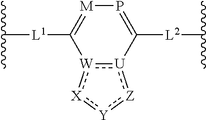

is

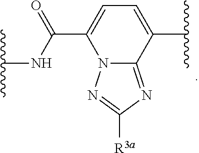

In some embodiments,

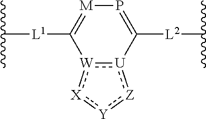

is

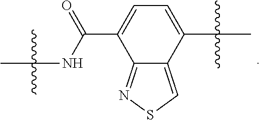 [SMILES: *NC(=O)c1ccc(*)c2csnc12]

In some embodiments, the compound of Formula (II) is a compound of Formula (II-b):

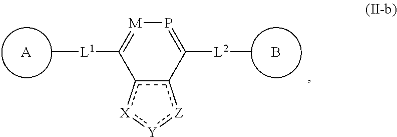

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; M and P are each independently C(R²) or N; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-c):

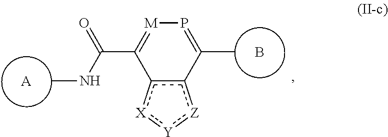

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; M and P are each independently C(R²) or N; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2. In some embodiments, the compound of Formula (II) is a compound of Formula (II-d):

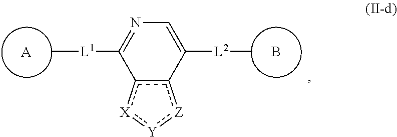

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2. In some embodiments, the compound of Formula (II) is a compound of Formula (II-e):

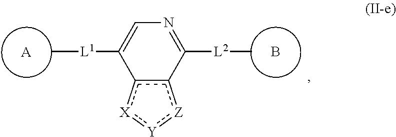

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2. In some embodiments, the compound of Formula (II) is a compound of Formula (II-f):

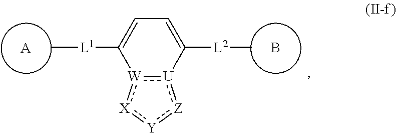

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; U and W are each independently C or N; X, Y, and Z are each independently C(R^{3a}), N, N(R^{3c}) or S, wherein at least one of X, Y, and Z is N or N(R^{3c}), and the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-g):

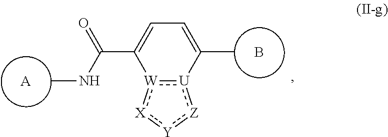

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; U and W are each independently C or N; X, Y, and Z are each independently C(R^{3a}), N, N(R^{3c}) or S, wherein at least one of X, Y, and Z is N or N(R^{3c}), and the bonds in the ring comprising U, W, X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2. In some embodiments, the compound of Formula (II) is a compound of Formula (II-h):

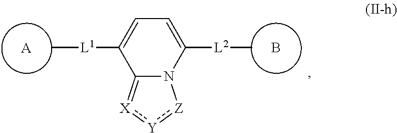

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2. In some embodiments, the compound of Formula (II) is a compound of Formula (II-i):

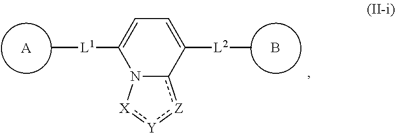

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2. In some embodiments, the compound of Formula (II) is a compound of Formula (II-j):

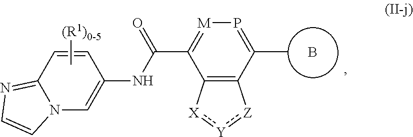

or a pharmaceutically acceptable salt, solvate, hydrate, tautomer, or stereoisomer thereof, B is cycloalkyl, heterocyclyl, aryl, or heteroaryl, each of which is optionally substituted with one or more R¹; X, Y, and Z are each independently C(R^{3a}) N, N(R^{3c}) or S, wherein at least one of X, Y, and Z is N or N(R^{3c}), and the bonds in the ring comprising X, Y, and Z may be single or double bonds as valency permits; M and P are each independently C(R²) or N; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-k):

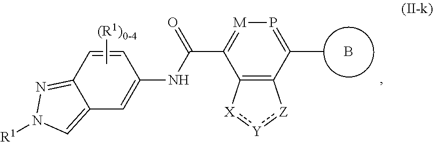

or a pharmaceutically acceptable salt, solvate, hydrate, tautomer, or stereoisomer thereof, B is cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; M and P are each independently C(R²) or N; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-l):

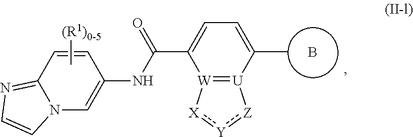

or a pharmaceutically acceptable salt, solvate, hydrate, tautomer, or stereoisomer thereof, B is cycloalkyl, heterocyclyl, aryl, or heteroaryl, each of which is optionally substituted with one or more R¹; U and W are each independently C or N; X, Y, and Z are each independently C(R^{3a}), N, N(R^{3c}) or S, wherein at least one of X, Y, and Z is N or N(R^{3c}), and the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-m):

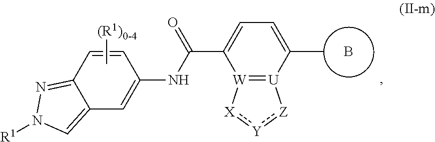

or a pharmaceutically acceptable salt, solvate, hydrate, tautomer, or stereoisomer thereof, B is cycloalkyl, heterocyclyl, aryl, or heteroaryl, each of which is optionally substituted with one or more R¹; U and W are each independently C or N; X, Y, and Z are each independently C(R^{3a}), N, N(R^{3c}) or S, wherein at least one of X, Y, and Z is N or N(R^{3c}), and the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is selected from a compound in Table 2, or a pharmaceutically acceptable salt thereof.

| CMPD | |
|---|---|
| No. | Structure |
| 900 | 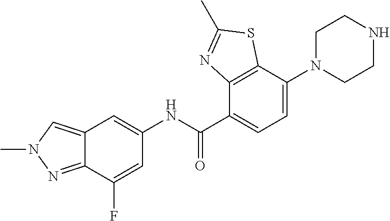 [SMILES: Cc1nc2c(C(=O)Nc3cc(F)c4nn(C)cc4c3)ccc(N3CCNCC3)c2s1] |
| 901 | 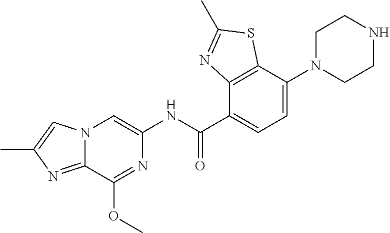 [SMILES: COc1nc(NC(=O)c2ccc(N3CCNCC3)c3sc(C)nc23)cn2cc(C)nc12] |
| 902 | 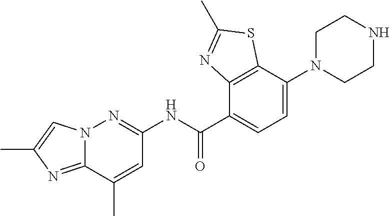 [SMILES: Cc1cn2nc(NC(=O)c3ccc(N4CCNCC4)c4sc(C)nc34)cc(C)c2n1] |
| 903 | 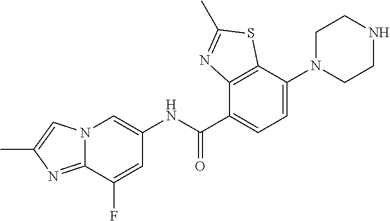 [SMILES: Cc1cn2cc(NC(=O)c3ccc(N4CCNCC4)c4sc(C)nc34)cc(F)c2n1] |
| 904 | 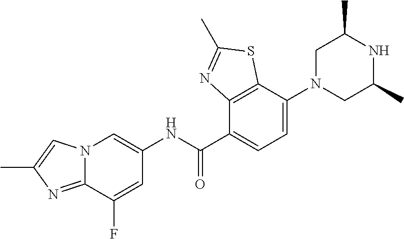 |
| 905 | 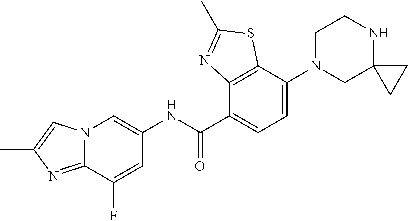 [SMILES: Cc1cn2cc(NC(=O)c3ccc(N4CCNC5(CC5)C4)c4sc(C)nc34)cc(F)c2n1] |
| 906 | 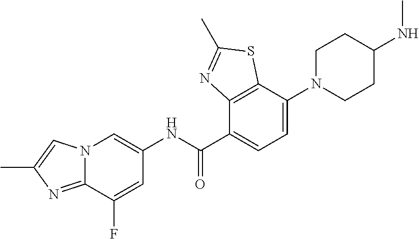 |
| 907 | 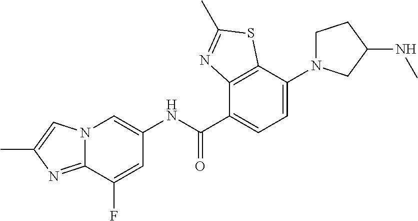 |
| 908 | 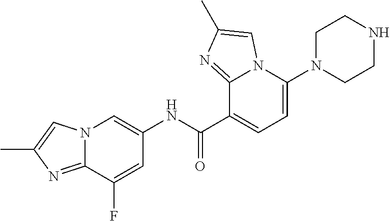 [SMILES: Cc1cn2cc(NC(=O)c3ccc(N4CCNCC4)n4cc(C)nc34)cc(F)c2n1] |
| 909 | 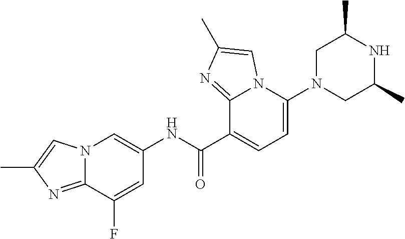 |
| 910 | 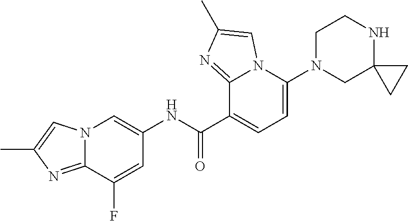 [SMILES: Cc1cn2cc(NC(=O)c3ccc(N4CCNC5(CC5)C4)n4cc(C)nc34)cc(F)c2n1] |
| 911 | 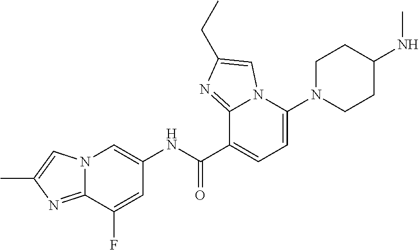 |
| 912 | 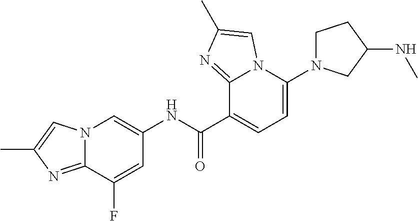 |
| 913 | 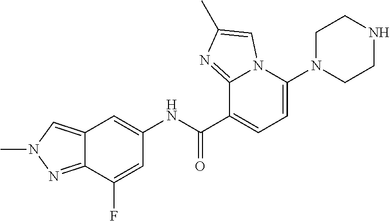 [SMILES: Cc1cn2c(N3CCNCC3)ccc(C(=O)Nc3cc(F)c4nn(C)cc4c3)c2n1] |
| 914 | 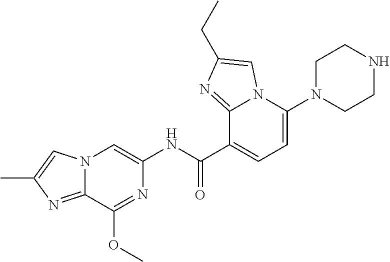 [SMILES: CCc1cn2c(N3CCNCC3)ccc(C(=O)Nc3cn4cc(C)nc4c(OC)n3)c2n1] |
| 915 | 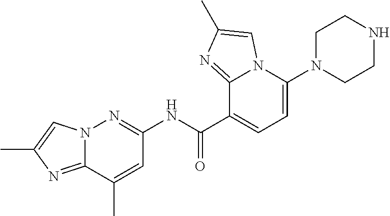 [SMILES: Cc1cn2nc(NC(=O)c3ccc(N4CCNCC4)n4cc(C)nc34)cc(C)c2n1] |
| 916 | 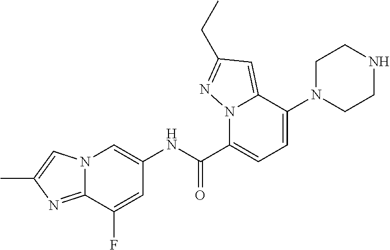 [SMILES: CCc1cc2c(N3CCNCC3)ccc(C(=O)Nc3cc(F)c4nc(C)cn4c3)n2n1] |
| 917 | 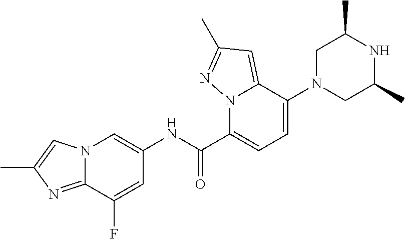 |
| 918 | 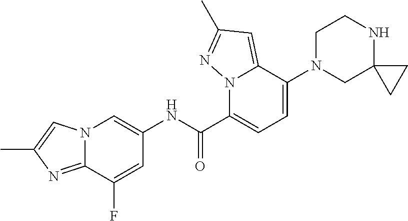 [SMILES: Cc1cn2cc(NC(=O)c3ccc(N4CCNC5(CC5)C4)c4cc(C)nn34)cc(F)c2n1] |
| 919 | 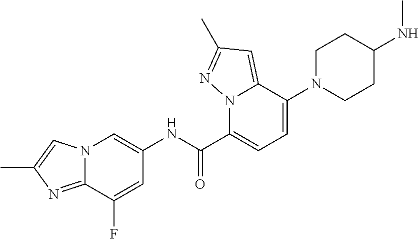 |
| 920 | 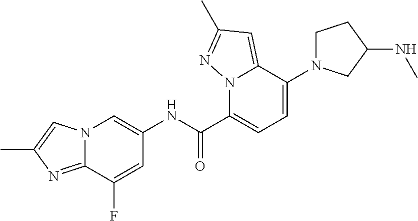 |
| 921 | 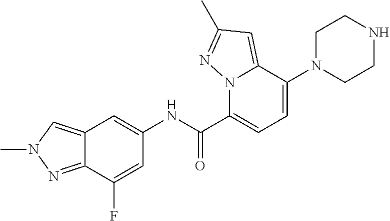 [SMILES: Cc1cc2c(N3CCNCC3)ccc(C(=O)Nc3cc(F)c4nn(C)cc4c3)n2n1] |
| 922 | 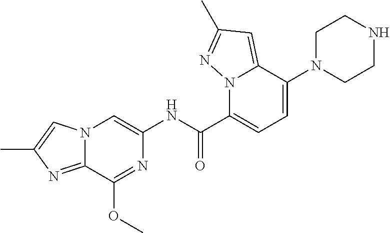 [SMILES: COc1nc(NC(=O)c2ccc(N3CCNCC3)c3cc(C)nn23)cn2cc(C)nc12] |
| 923 | 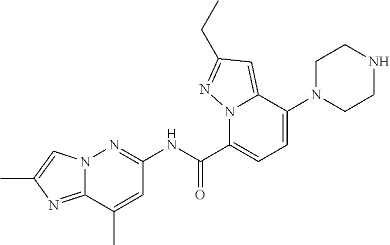 [SMILES: CCc1cc2c(N3CCNCC3)ccc(C(=O)Nc3cc(C)c4nc(C)cn4n3)n2n1] |
| 924 | 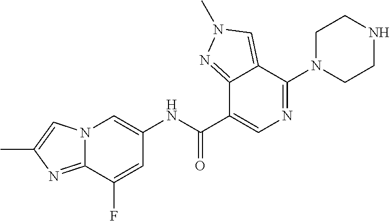 [SMILES: Cc1cn2cc(NC(=O)c3cnc(N4CCNCC4)c4cn(C)nc34)cc(F)c2n1] |
| 925 | 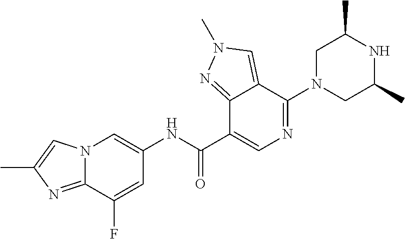 |
| 926 | 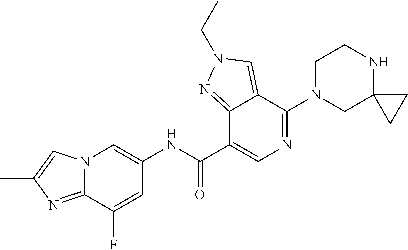 [SMILES: CCn1cc2c(N3CCNC4(CC4)C3)ncc(C(=O)Nc3cc(F)c4nc(C)cn4c3)c2n1] |
| 927 | 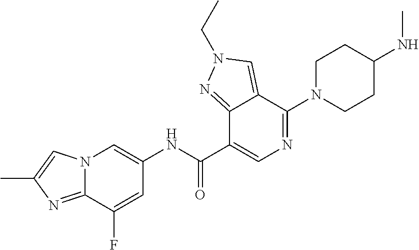 |
| 928 | 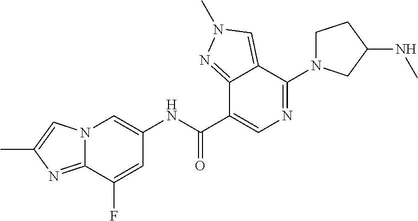 |
| 929 | 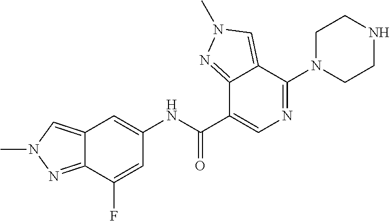 [SMILES: Cn1cc2cc(NC(=O)c3cnc(N4CCNCC4)c4cn(C)nc34)cc(F)c2n1] |
| 930 | 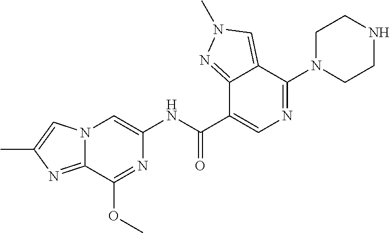 [SMILES: COc1nc(NC(=O)c2cnc(N3CCNCC3)c3cn(C)nc23)cn2cc(C)nc12] |
| 931 | 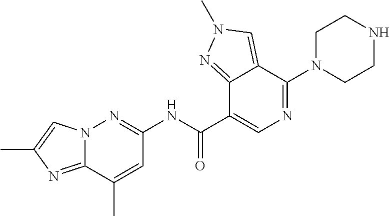 [SMILES: Cc1cn2nc(NC(=O)c3cnc(N4CCNCC4)c4cn(C)nc34)cc(C)c2n1] |
| 932 | 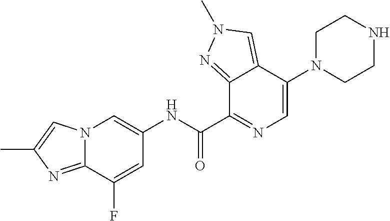 [SMILES: Cc1cn2cc(NC(=O)c3ncc(N4CCNCC4)c4cn(C)nc34)cc(F)c2n1] |
| 933 | 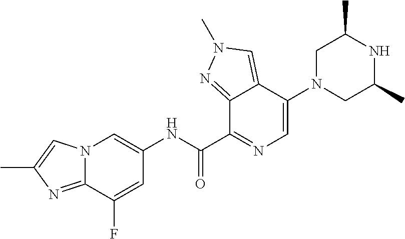 |
| 934 | 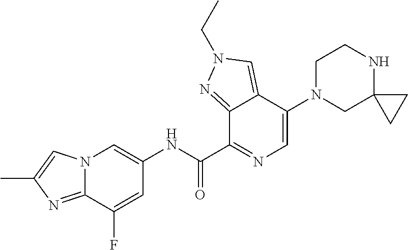 [SMILES: CCn1cc2c(N3CCNC4(CC4)C3)cnc(C(=O)Nc3cc(F)c4nc(C)cn4c3)c2n1] |
| 935 | 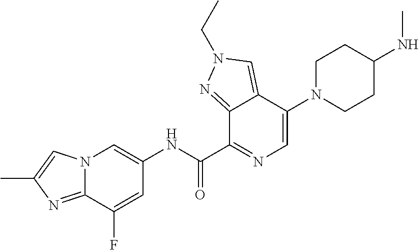 |
| 936 | 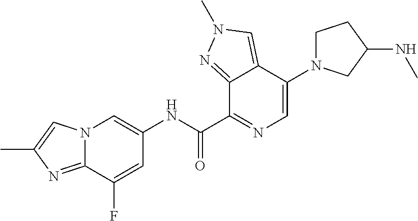 |
| 937 | 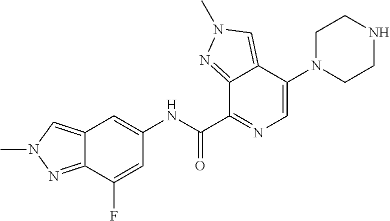 [SMILES: Cn1cc2cc(NC(=O)c3ncc(N4CCNCC4)c4cn(C)nc34)cc(F)c2n1] |
| 938 | 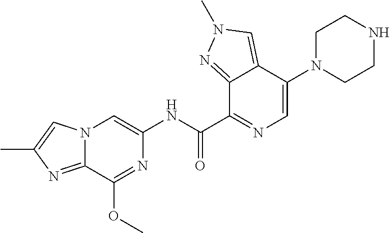 [SMILES: COc1nc(NC(=O)c2ncc(N3CCNCC3)c3cn(C)nc23)cn2cc(C)nc12] |
| 939 | 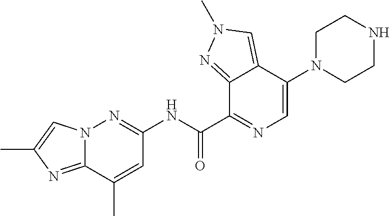 [SMILES: Cc1cn2nc(NC(=O)c3ncc(N4CCNCC4)c4cn(C)nc34)cc(C)c2n1] |
| 940 | 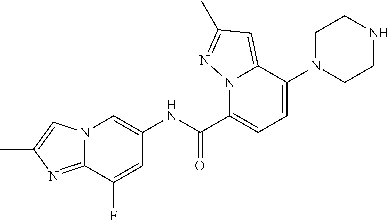 [SMILES: Cc1cn2cc(NC(=O)c3ccc(N4CCNCC4)c4cc(C)nn34)cc(F)c2n1] |
| 941 | 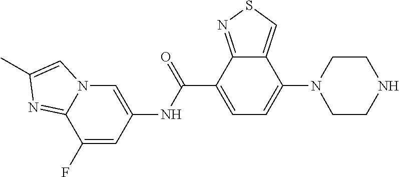 [SMILES: Cc1cn2cc(NC(=O)c3ccc(N4CCNCC4)c4csnc34)cc(F)c2n1] |
| 942 | 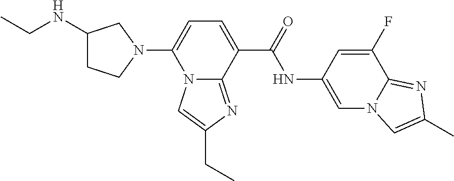 [SMILES: CCNC1CCN(c2ccc(C(=O)Nc3cc(F)c4nc(C)cn4c3)c3nc(CC)cn23)C1] |
| 943 | 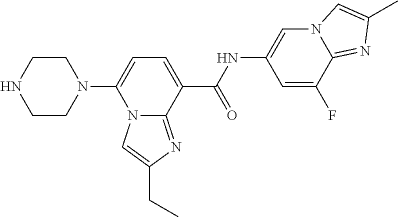 [SMILES: CCc1cn2c(N3CCNCC3)ccc(C(=O)Nc3cc(F)c4nc(C)cn4c3)c2n1] |
| 944 | 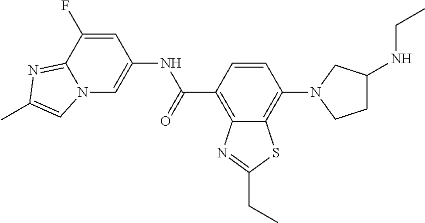 [SMILES: CCNC1CCN(c2ccc(C(=O)Nc3cc(F)c4nc(C)cn4c3)c3nc(CC)sc23)C1] |
| 945 | 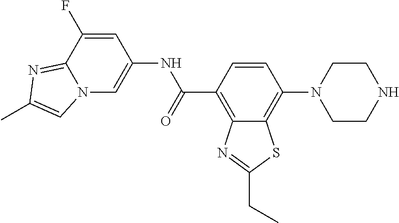 [SMILES: CCc1nc2c(C(=O)Nc3cc(F)c4nc(C)cn4c3)ccc(N3CCNCC3)c2s1] |
| 946 | 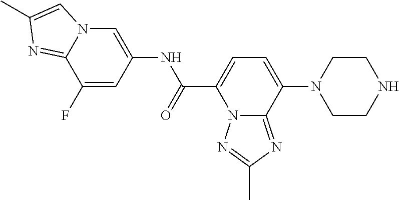 [SMILES: Cc1cn2cc(NC(=O)c3ccc(N4CCNCC4)c4nc(C)nn34)cc(F)c2n1] |
| 947 | 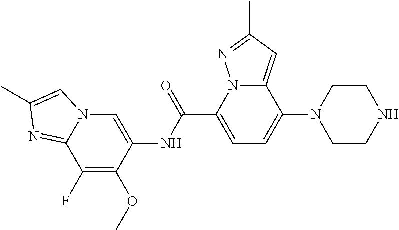 [SMILES: COc1c(NC(=O)c2ccc(N3CCNCC3)c3cc(C)nn23)cn2cc(C)nc2c1F] |
| 948 | 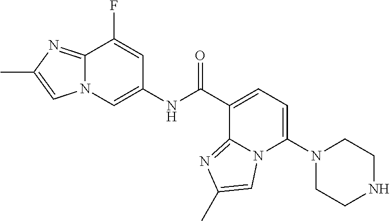 [SMILES: Cc1cn2cc(NC(=O)c3ccc(N4CCNCC4)n4cc(C)nc34)cc(F)c2n1] |
| 949 | 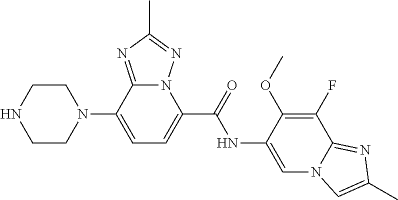 [SMILES: COc1c(NC(=O)c2ccc(N3CCNCC3)c3nc(C)nn23)cn2cc(C)nc2c1F] |
| 950 | 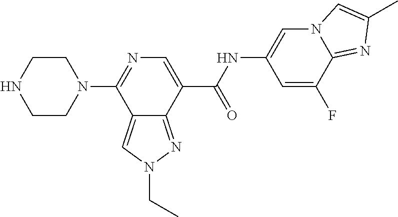 [SMILES: CCn1cc2c(N3CCNCC3)ncc(C(=O)Nc3cc(F)c4nc(C)cn4c3)c2n1] |
| 951 | 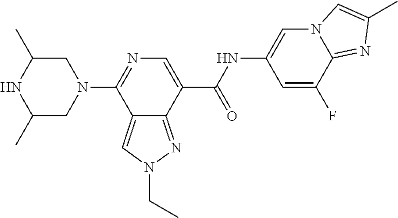 [SMILES: CCn1cc2c(N3CC(C)NC(C)C3)ncc(C(=O)Nc3cc(F)c4nc(C)cn4c3)c2n1] |
| 952 | 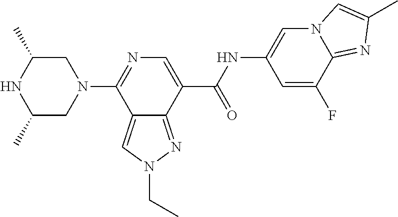 [SMILES: CCn1cc2c(N3C[C@@H](C)N[C@@H](C)C3)ncc(C(=O)Nc3cc(F)c4nc(C)cn4c3)c2n1] |
| 953 | 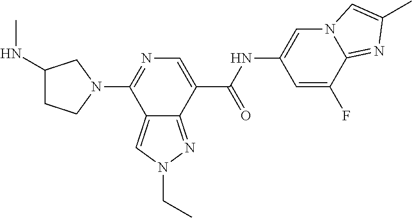 [SMILES: CCn1cc2c(N3CCC(NC)C3)ncc(C(=O)Nc3cc(F)c4nc(C)cn4c3)c2n1] |
| 954 | 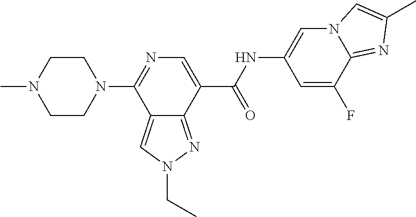 |
| 955 | 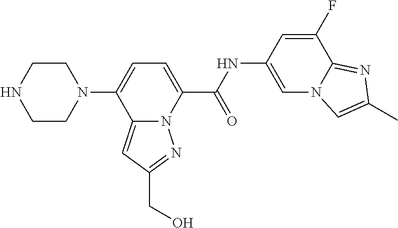 [SMILES: Cc1cn2cc(NC(=O)c3ccc(N4CCNCC4)c4cc(CO)nn34)cc(F)c2n1] |
| 956 | 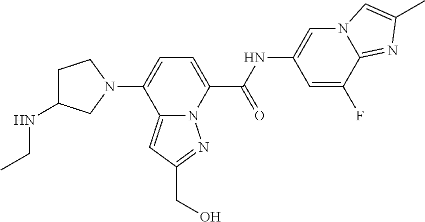 [SMILES: CCNC1CCN(c2ccc(C(=O)Nc3cc(F)c4nc(C)cn4c3)n3nc(CO)cc23)C1] |
| 957 | 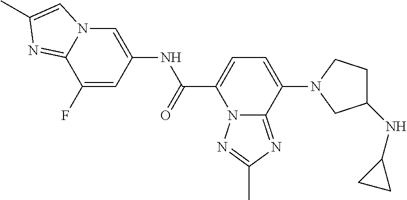 [SMILES: Cc1cn2cc(NC(=O)c3ccc(N4CCC(NC5CC5)C4)c4nc(C)nn34)cc(F)c2n1] |
| 958 | 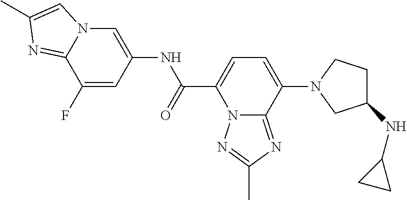 [SMILES: Cc1cn2cc(NC(=O)c3ccc(N4CC[C@@H](NC5CC5)C4)c4nc(C)nn34)cc(F)c2n1] |
| 959 | 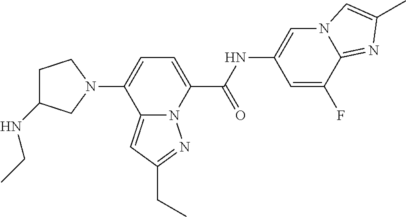 [SMILES: CCNC1CCN(c2ccc(C(=O)Nc3cc(F)c4nc(C)cn4c3)n3nc(CC)cc23)C1] |
| 960 | 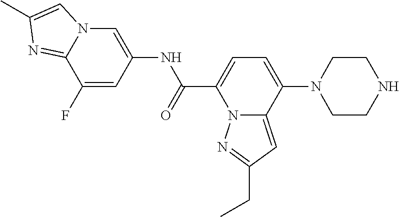 [SMILES: CCc1cc2c(N3CCNCC3)ccc(C(=O)Nc3cc(F)c4nc(C)cn4c3)n2n1] |
| 961 | 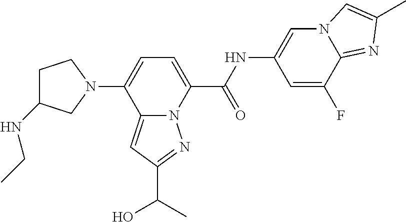 [SMILES: CCNC1CCN(c2ccc(C(=O)Nc3cc(F)c4nc(C)cn4c3)n3nc(C(C)O)cc23)C1] |
| 962 | 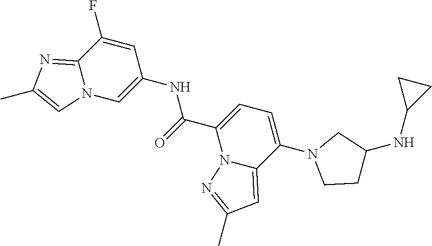 [SMILES: Cc1cn2cc(NC(=O)c3ccc(N4CCC(NC5CC5)C4)c4cc(C)nn34)cc(F)c2n1] |
| 963 | 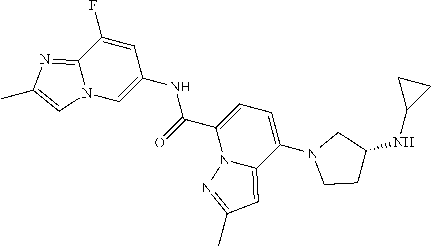 [SMILES: Cc1cn2cc(NC(=O)c3ccc(N4CC[C@@H](NC5CC5)C4)c4cc(C)nn34)cc(F)c2n1] |
| 964 | 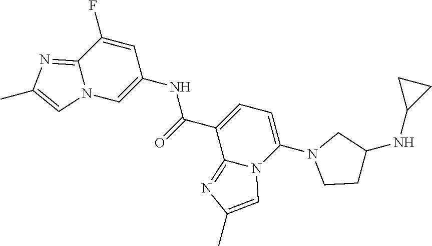 [SMILES: Cc1cn2cc(NC(=O)c3ccc(N4CCC(NC5CC5)C4)n4cc(C)nc34)cc(F)c2n1] |
| 965 | 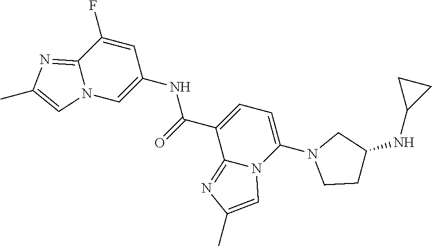 [SMILES: Cc1cn2cc(NC(=O)c3ccc(N4CC[C@@H](NC5CC5)C4)n4cc(C)nc34)cc(F)c2n1] |
| 966 | 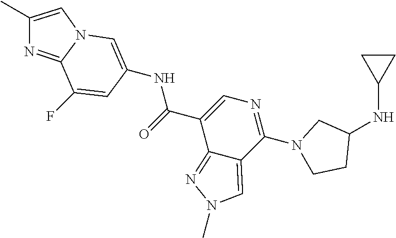 [SMILES: Cc1cn2cc(NC(=O)c3cnc(N4CCC(NC5CC5)C4)c4cn(C)nc34)cc(F)c2n1] |
| 967 | 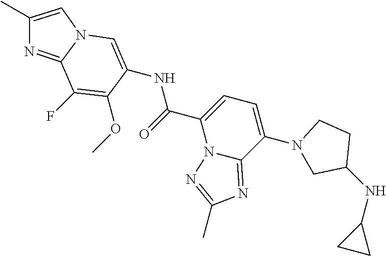 [SMILES: COc1c(NC(=O)c2ccc(N3CCC(NC4CC4)C3)c3nc(C)nn23)cn2cc(C)nc2c1F] |
| 968 | 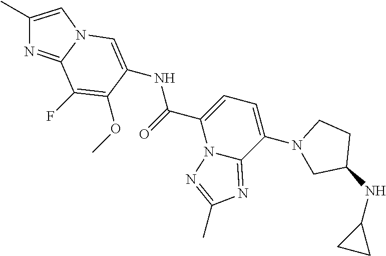 [SMILES: COc1c(NC(=O)c2ccc(N3CC[C@@H](NC4CC4)C3)c3nc(C)nn23)cn2cc(C)nc2c1F] |
| 969 | 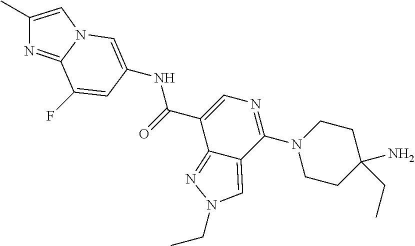 [SMILES: CCn1cc2c(N3CCC(N)(CC)CC3)ncc(C(=O)Nc3cc(F)c4nc(C)cn4c3)c2n1] |
| 970 | 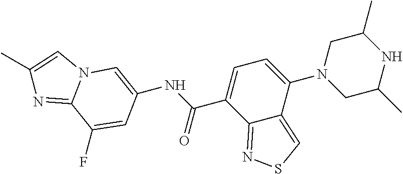 |
| 971 | 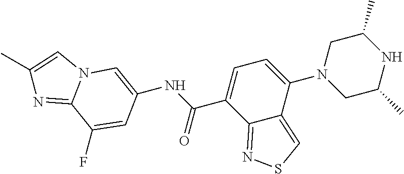 |
| 972 | 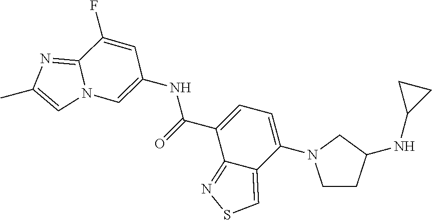 [SMILES: Cc1cn2cc(NC(=O)c3ccc(N4CCC(NC5CC5)C4)c4csnc34)cc(F)c2n1] |
| 973 | 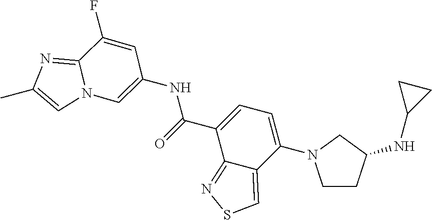 [SMILES: Cc1cn2cc(NC(=O)c3ccc(N4CC[C@@H](NC5CC5)C4)c4csnc34)cc(F)c2n1] |
| 974 | 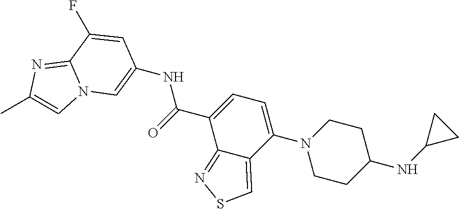 [SMILES: Cc1cn2cc(NC(=O)c3ccc(N4CCC(NC5CC5)CC4)c4csnc34)cc(F)c2n1] |
| 975 | 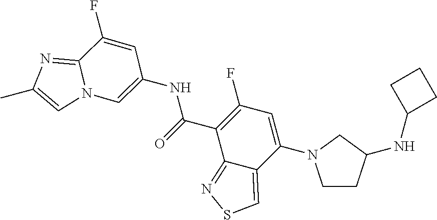 [SMILES: Cc1cn2cc(NC(=O)c3c(F)cc(N4CCC(NC5CCC5)C4)c4csnc34)cc(F)c2n1] |
| 976 | 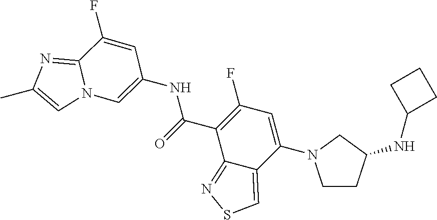 [SMILES: Cc1cn2cc(NC(=O)c3c(F)cc(N4CC[C@@H](NC5CCC5)C4)c4csnc34)cc(F)c2n1] |
| 977 | 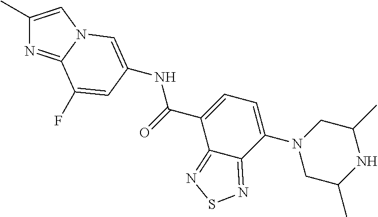 |
| 978 | 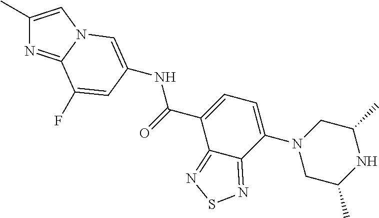 |
| 979 | 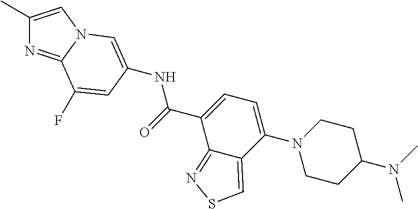 |
| 980 | 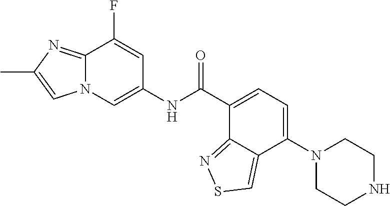 [SMILES: Cc1cn2cc(NC(=O)c3ccc(N4CCNCC4)c4csnc34)cc(F)c2n1] |
| 981 | 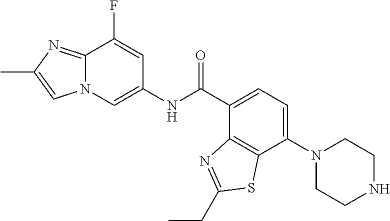 [SMILES: CCc1nc2c(C(=O)Nc3cc(F)c4nc(C)cn4c3)ccc(N3CCNCC3)c2s1] |
| 982 | 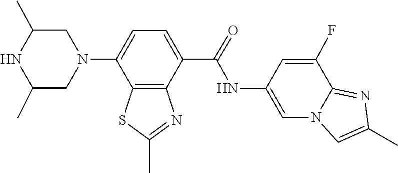 [SMILES: Cc1cn2cc(NC(=O)c3ccc(N4CC(C)NC(C)C4)c4sc(C)nc34)cc(F)c2n1] |
| 983 | 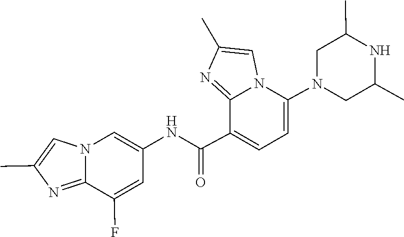 |
| 984 | 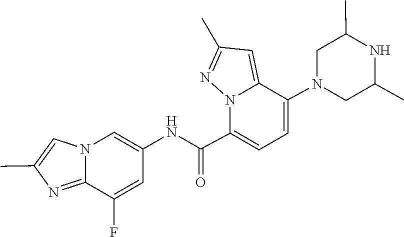 |
| 985 | 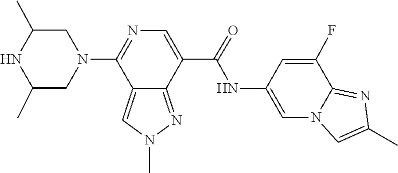 [SMILES: Cc1cn2cc(NC(=O)c3cnc(N4CC(C)NC(C)C4)c4cn(C)nc34)cc(F)c2n1] |
| 986 | 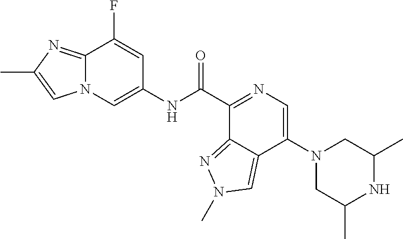 |
| 987 | 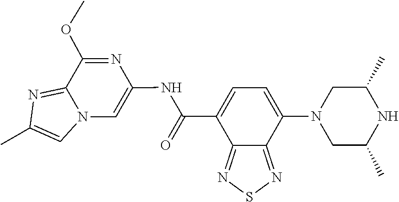 [SMILES: COc1nc(NC(=O)c2ccc(N3C[C@@H](C)N[C@@H](C)C3)c3nsnc23)cn2cc(C)nc12] |
| 988 | 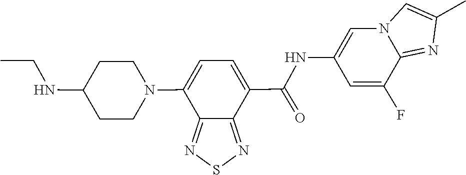 [SMILES: CCNC1CCN(c2ccc(C(=O)Nc3cc(F)c4nc(C)cn4c3)c3nsnc23)CC1] |
| 989 | 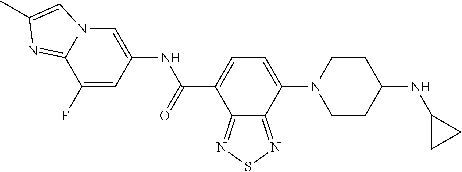 [SMILES: Cc1cn2cc(NC(=O)c3ccc(N4CCC(NC5CC5)CC4)c4nsnc34)cc(F)c2n1] |
| 990 | 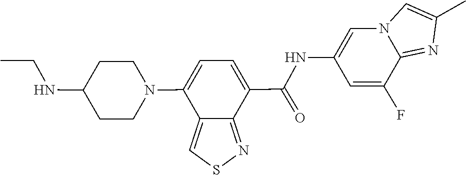 [SMILES: CCNC1CCN(c2ccc(C(=O)Nc3cc(F)c4nc(C)cn4c3)c3nscc23)CC1] |
| 991 | 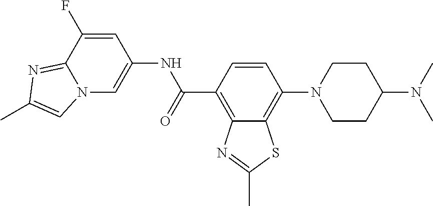 |
| 992 | 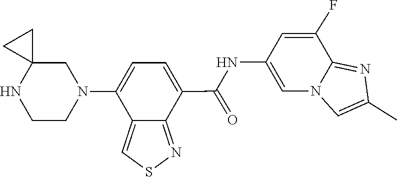 [SMILES: Cc1cn2cc(NC(=O)c3ccc(N4CCNC5(CC5)C4)c4csnc34)cc(F)c2n1] |
| 993 | 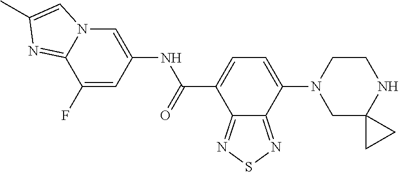 [SMILES: Cc1cn2cc(NC(=O)c3ccc(N4CCNC5(CC5)C4)c4nsnc34)cc(F)c2n1] |
| 994 | 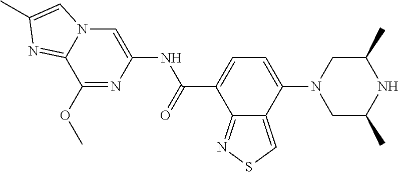 |
| 995 | 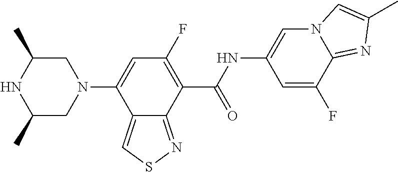 |
| 996 | 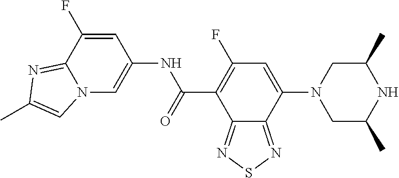 |
| 997 | 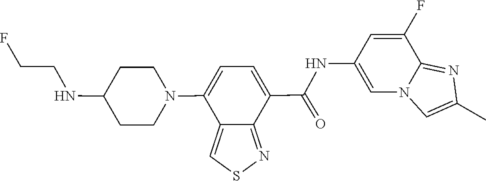 [SMILES: Cc1cn2cc(NC(=O)c3ccc(N4CCC(NCCF)CC4)c4csnc34)cc(F)c2n1] |
| 998 | 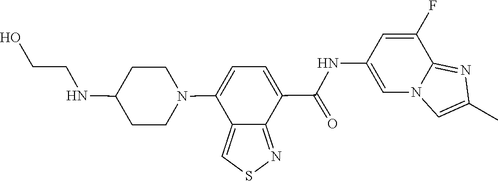 [SMILES: Cc1cn2cc(NC(=O)c3ccc(N4CCC(NCCO)CC4)c4csnc34)cc(F)c2n1] |
| 999 | 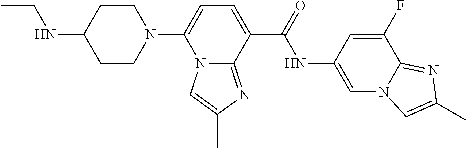 [SMILES: CCNC1CCN(c2ccc(C(=O)Nc3cc(F)c4nc(C)cn4c3)c3nc(C)cn23)CC1] |
| 1000 | 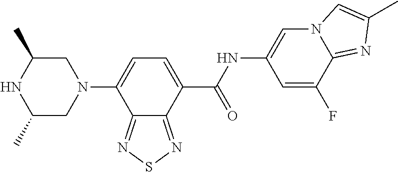 |
| 1001 | 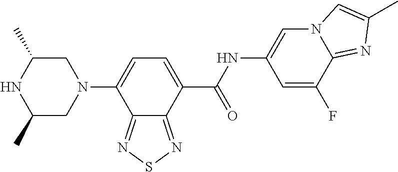 |
| 1002 | 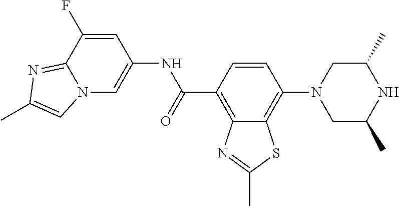 |
| 1003 | 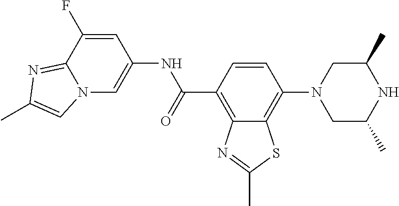 |
| 1004 | 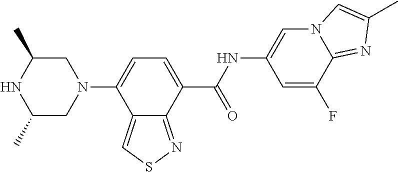 |
| 1005 | 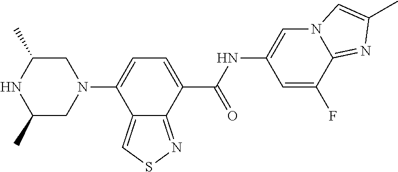 |
| 1006 | 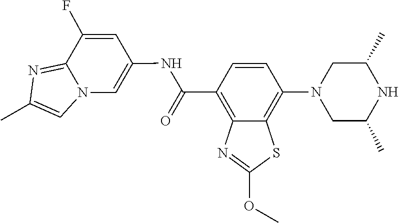 [SMILES: COc1nc2c(C(=O)Nc3cc(F)c4nc(C)cn4c3)ccc(N3C[C@@H](C)N[C@@H](C)C3)c2s1] |
| 1007 | 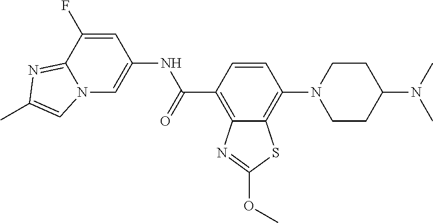 |
| 1008 | 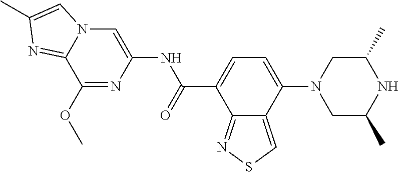 |
| 1009 | 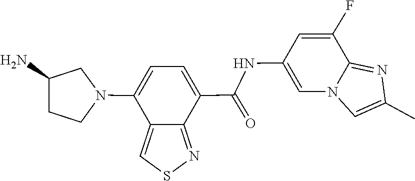 [SMILES: Cc1cn2cc(NC(=O)c3ccc(N4CC[C@@H](N)C4)c4csnc34)cc(F)c2n1] |
| 1010 | 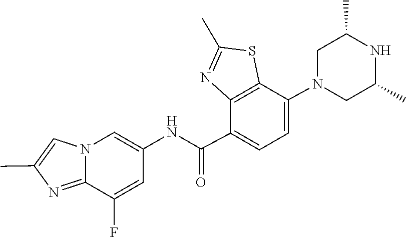 |
| 1011 | 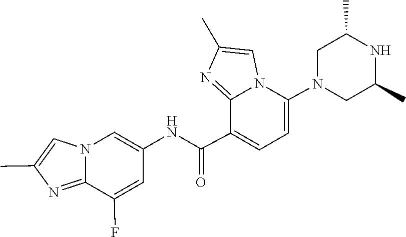 |
| 1012 | 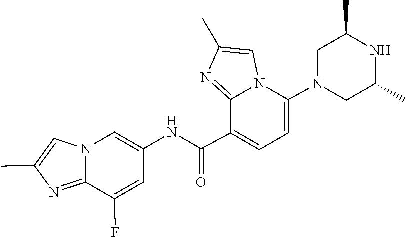 |
| 1013 | 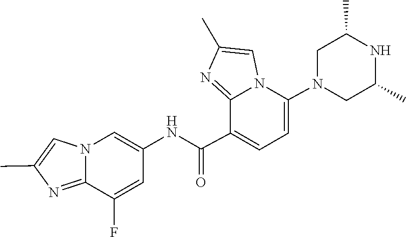 |
| 1014 | 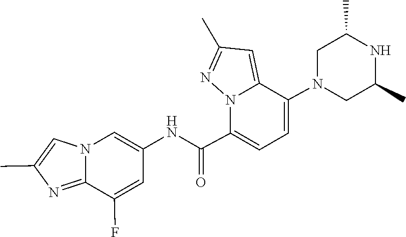 |
| 1015 | 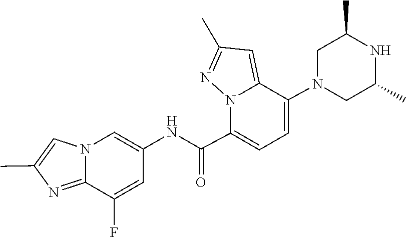 |
| 1016 | 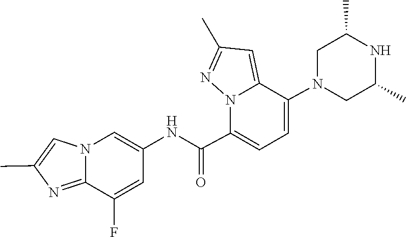 |
| 1017 | 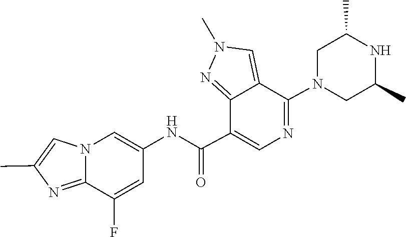 |
| 1018 | 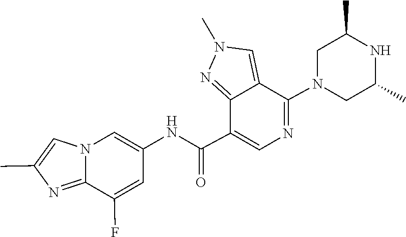 |
| 1019 | 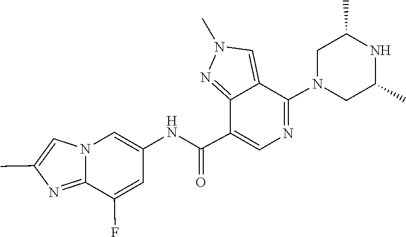 |
| 1020 | 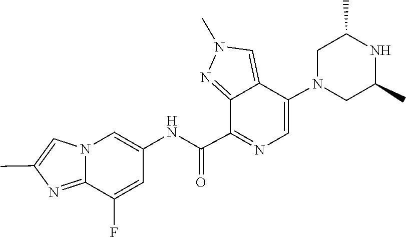 |
| 1021 | 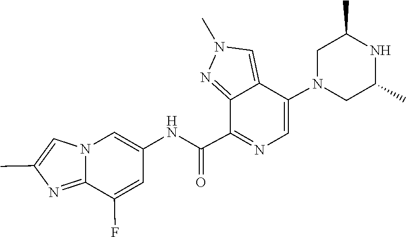 |
| 1022 | 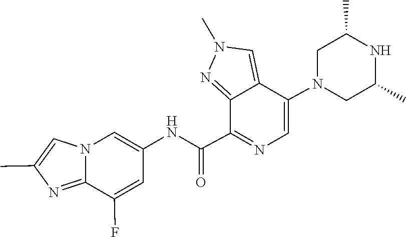 |
| 1023 | 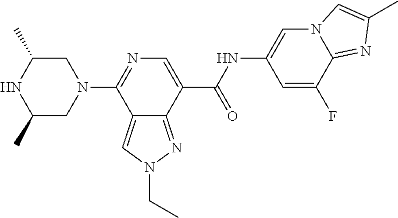 |
| 1024 | 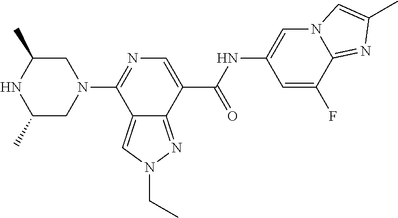 |
| 1025 | 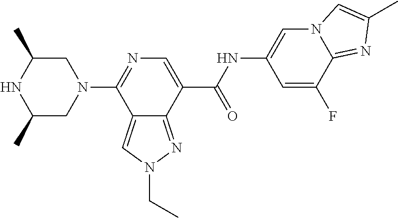 |
| 1026 | 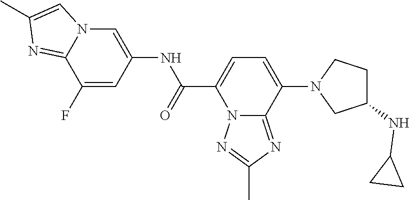 [SMILES: Cc1cn2cc(NC(=O)c3ccc(N4CC[C@H](NC5CC5)C4)c4nc(C)nn34)cc(F)c2n1] |
| 1027 | 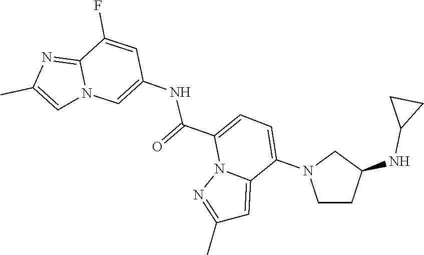 [SMILES: Cc1cn2cc(NC(=O)c3ccc(N4CC[C@H](NC5CC5)C4)c4cc(C)nn34)cc(F)c2n1] |
| 1028 | 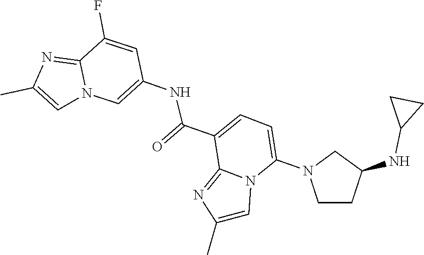 [SMILES: Cc1cn2cc(NC(=O)c3ccc(N4CC[C@H](NC5CC5)C4)n4cc(C)nc34)cc(F)c2n1] |
| 1029 | 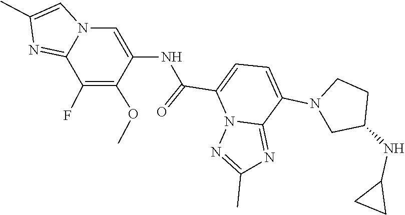 [SMILES: COc1c(NC(=O)c2ccc(N3CC[C@H](NC4CC4)C3)c3nc(C)nn23)cn2cc(C)nc2c1F] |
| 1030 | 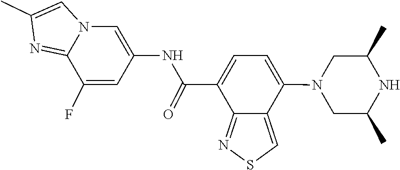 |
| 1031 | 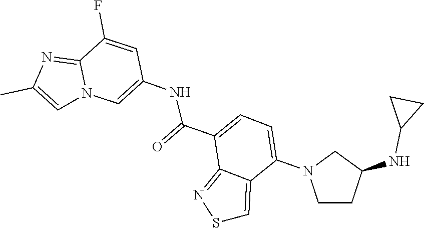 [SMILES: Cc1cn2cc(NC(=O)c3ccc(N4CC[C@H](NC5CC5)C4)c4csnc34)cc(F)c2n1] |
| 1032 | 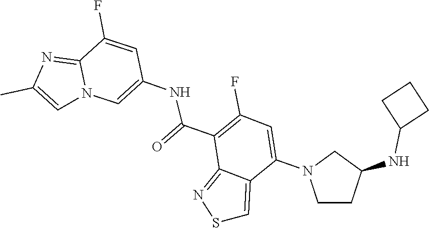 [SMILES: Cc1cn2cc(NC(=O)c3c(F)cc(N4CC[C@H](NC5CCC5)C4)c4csnc34)cc(F)c2n1] |
| 1033 | 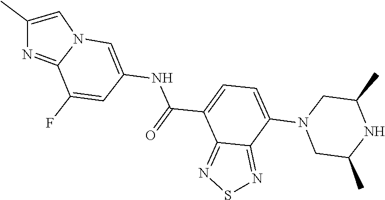 |
| 1034 | 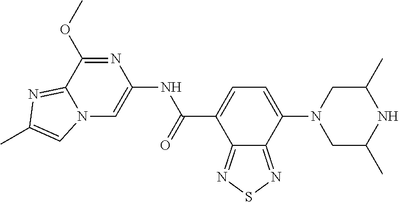 |
| 1035 | 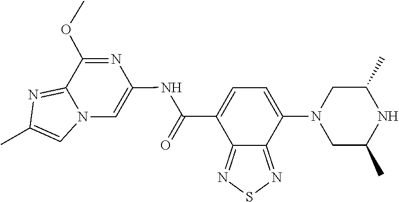 [SMILES: COc1nc(NC(=O)c2ccc(N3C[C@H](C)N[C@@H](C)C3)c3nsnc23)cn2cc(C)nc12] |
| 1036 | 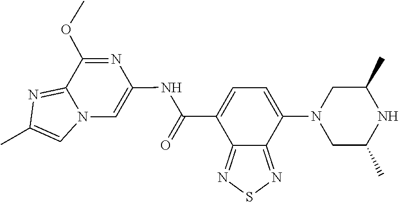 [SMILES: COc1nc(NC(=O)c2ccc(N3C[C@@H](C)N[C@H](C)C3)c3nsnc23)cn2cc(C)nc12] |
| 1037 | 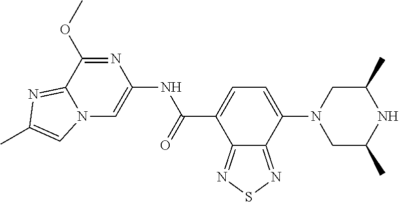 |
| 1038 | 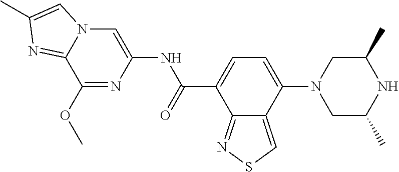 |
| 1039 | 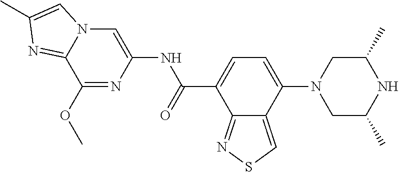 |
| 1040 | 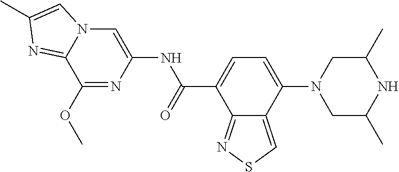 |
| 1041 | 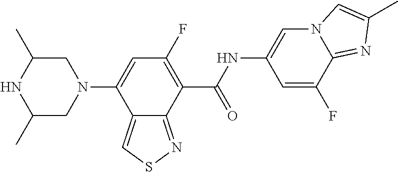 |
| 1042 | 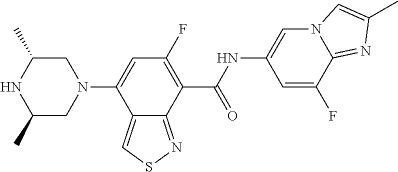 |
| 1043 | 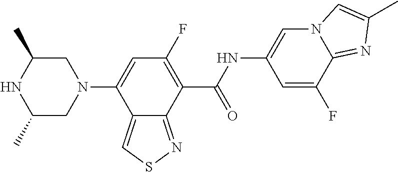 |
| 1044 | 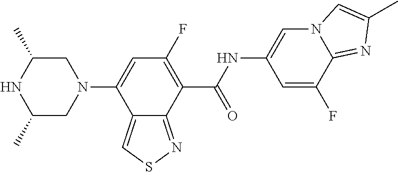 |
| 1045 | 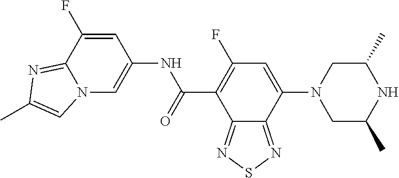 |
| 1046 | 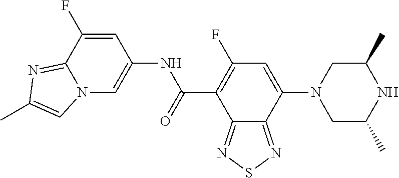 |
| 1047 | 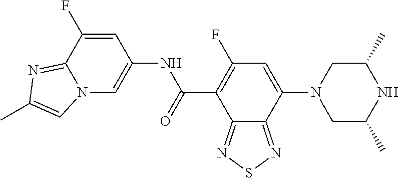 |
| 1048 | 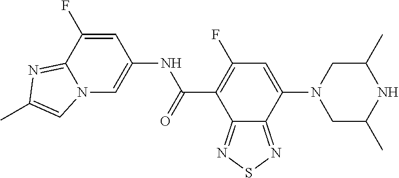 |
| 1049 | 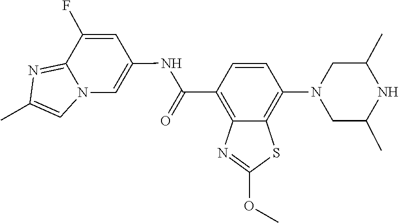 [SMILES: COc1nc2c(C(=O)Nc3cc(F)c4nc(C)cn4c3)ccc(N3CC(C)NC(C)C3)c2s1] |
| 1050 | 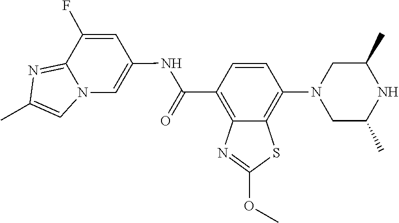 [SMILES: COc1nc2c(C(=O)Nc3cc(F)c4nc(C)cn4c3)ccc(N3C[C@@H](C)N[C@H](C)C3)c2s1] |
| 1051 | 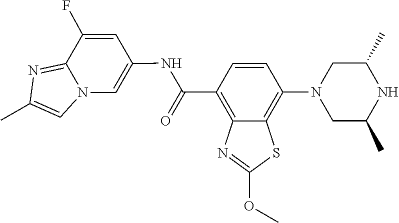 [SMILES: COc1nc2c(C(=O)Nc3cc(F)c4nc(C)cn4c3)ccc(N3C[C@H](C)N[C@@H](C)C3)c2s1] |
| 1052 | 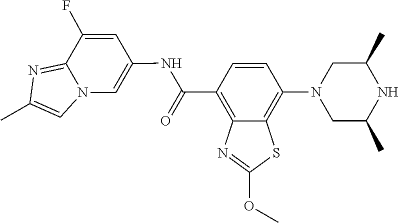 [SMILES: COc1nc2c(C(=O)Nc3cc(F)c4nc(C)cn4c3)ccc(N3C[C@@H](C)N[C@@H](C)C3)c2s1] |
| 1053 | 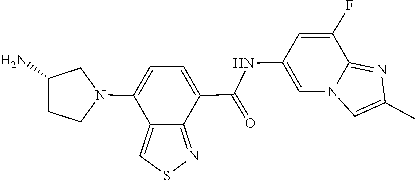 [SMILES: Cc1cn2cc(NC(=O)c3ccc(N4CC[C@H](N)C4)c4csnc34)cc(F)c2n1] |
| 1054 | 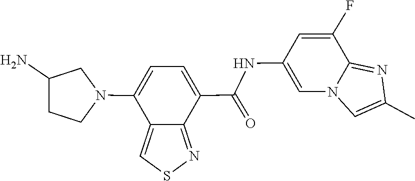 [SMILES: Cc1cn2cc(NC(=O)c3ccc(N4CCC(N)C4)c4csnc34)cc(F)c2n1] |

### Pharmaceutical Compositions, Kits, and Administration

The present invention provides pharmaceutical compositions comprising a compound of Formula (I) or (II), e.g., a compound of Formula (I) or (II) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I) or (II) or a pharmaceutically acceptable salt thereof, and optionally a pharmaceutically acceptable excipient. In certain embodiments, the compound of Formula (I) or (II) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I) or (II) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I) or (II) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### METHODS OF USE

Described herein are compounds useful for modulating splicing. In some embodiments, a compound of Formula (I) or (II) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I) or (II) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I) or (II). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I) or (II). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I) or (II), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAMJS, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNVA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASHIL-IT1, ASNSD1, ASPM, ATAD5, ATF1, ATG4A, ATG16L2, ATM, ATN1, ATP11C, ATP6VJG3, ATP13A5, ATP7A, ATP7B, ATR, ATXN2, ATXN3, ATXN7, ATXN10, AXIN1, B2M, B4GALNT3, BBS4, BCL2, BCL2L1, BCL2-like 11 (BIM), BCL11B, BBOX1, BCSIL, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf1l, C14orf118, C15orf29, C15orf42, C15orf6O, C16orf33, C16orf38, C16orf48, C18orf8, C19orf42, C1orf107, C1orf114, C1orf130, C1orf149, C1orf27, C1orf71, C1orf94, C1R, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMKID, CAMKK1, CAPN3, CAPN9, CAPSL, CARD11, CARKD, CASZl, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENP1, CENPT, CENTB2, CENTG2, CEPJ10, CEP170, CEP192, CETP, CFB, CFTR, CFH, CGN, CGNL1, CHAF1A, CHD9, CHIC2, CHL1, CHN1, CHM, CLEC16A, CLIC2, CLCN1, CLINT1, CLK1, CLPB, CLP™1, CMIP, CMYA5, CNGA3, CNOT1, CNOT7, CNTN6, COG3, COLIJA1, COL11A2, COL12A1, COL14A1, COL15A1, COL17A1, COL19A1, COL1A1, COL1A2, COL2A1, COL3A1, COL4A1, COL4A2, COL4A5, COL4A6, COL5A2, COL6A1, COL7A, COL9A, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF1, DNAH3, DNAH8, DNAI1, DNAJA4, DNAJC13, DNAJC7, DNMTl, DNTTIP2, DOCK4, DOCK5, DOCK10, DOCK11, DOT1L, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBNl, FBX015, FBX018, FBXO38, FCGBP, FECH, FEZ2, FGA, FGD6, FGFR2, FGFR10P, FGFR10P2, FGFR2, FGG, FGR, FIX, FKBP3, FLI1, FLJ35848, FLJ36070, FLNA, EN1, FNBP1L, FOLH1, FOSL1, FOSL2, FOXK1, FOAM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GH1, GHR, GHV, GJA1, GLA, GLT8D1, GNA11, GNAQ, GNAS, GNB5, GOLGB1, GOLTIA, GOLT1B, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF21, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXCO, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF, IKZF3, ILIR2, IL5RA, IL7RA, IMAIT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, ITGB3, ITGB4, ITIH1, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIFSA, KIFSB, KIF9, KIN, KIR2DL5B, KIR3DL2, KIR3DL3, KIT, KLF3, KLF5, KLF7, KLF0, KLF12, KLF16, KLHL20, KLK12, KLKB1, KMT2A, KMT2B, KPNA5, KRAS, KREMEN1, KRIT1, KRT5, KRTCAP2, KYNU, LICAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC, MARCH5, MATN2, MBD3, MCF2L2, MCM6, MDGA2, MDM4, ASXL1, FUS, SPR54, MECOM, MEF2C, MEF2D, MEGF0, MEGF1, MEMO1, MET, MGA, MGAM, MGAT4A, MGAT5, MGC16169, MGC34774, MKKS, MIB1, MIER2, MITF, MKL2, MLANA, MLH1, MLL5, MLXA MAE, MPDZ, MP1, MRAP2, MRPLI1, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK, NEK5, NEK1, NF, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOLI0, NOP561, NOS1, NOS2A, NOTCH1, NPAS4, NPM1, NRID1, NRIH3, NR1H4, NR4A3, NR5A1, NRXN1, NSMAF, NSMCE2, NTSC, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPAl, OPN4, OPTN, OSBPLI1, OSBPL8, OSGEPL1, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM, PBX2, PCBP4, PCCA, PCGF2, PCNX, PCOTH, PDCD4, PDE4D, PDE8B, PDE10A, PD1A3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF, PIAS1, PIEZO1, PIGF, PIGN, PIGT, PIK3C2G, PIK3CA, PIK3CD, PIK3CG, PIK3R1, PIP5KIA, PITRM1, PIWIL3, PKD1, PKHDIL1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPFIA2, PPPIR12A, PPP3CB, PPP4C, PPP4R1L, PPP4R2, PRAME, PRC1, PRDM1, PREX1, PREX2, PRIM1, PRIM2, PRKARIA, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPNI1, PTPN22, PTPRD, PTPRK, PTPRM, PTPRN2, PTPRT, PUS10, PVRL2, PYGM, QRSL1, RABI1FIP2, RAB23, RAF1, RALBP1, RALGDS, RBICC1, RBL2, RBM39, RBM45, RBPJ, RBSN, REC8, RELB, RFC4, RFT1, RFTN1, RHOA, RHPN2, RIF1, RIT1, RLN3, RMND5B, RNFI1, RNF32, RNFT1, RNGTT, ROCK1, ROCK2, RORA, RP1, RP6KA3, RP11-265F1, RP13-36C9, RPAP3, RPN1, RPGR, RPL22, RPL22L1, RPS6KA6, RREB1, RRM1, RRPIB, RSK2, RTEL1, RTF1, RUFY1, RUNX1, RUNX2, RXRA, RYR3, SAAL1, SAE1, SALL4, SAT1, SATB2, SBCAD, SCNIA, SCN2A, SCN3A, SCN4A, SCN5A, SCN8A, SCNA, SCNIJA, SCO1, SCYL3, SDC1, SDK1, SDK2, SEC24A, SEC24D, SEC31A, SELIL, SENP3, SENP6, SENP7, SERPINA1, SETD3, SETD4, SETDB1, SEZ6, SFRS12, SGCE, SGOL2, SGPL1, SH2DIA, SH3BGRL2, SH3PXD2A, SH3PXD2B, SH3RF2, SH3TC2, SHOC2, SIPAIL2, SIPAIL3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX SMS, SMTN, SNCAIP, SNORD86, SNRK, SNRP70, SNX5, SNX6, SOD1, SOD10, SOS, SOS2, SOX5, SOX6, SOX8, SP1, SP2, SP3, SPI10, SPAG9, SPATA13, SPATA4, SPATS1, SPECC1L, SPDEF, SPI1, SPINK5, SPP2, SPTA1, SRF, SRM, SRP72, SSX3, SSX5, SSX9, STAG1, STAG2, STAMBPL1, STARD6, STAT1, STAT3, STAT5A, STAT5B, STAT6, STK17B, STX3, STXBP1, SUCLG2, SULF2, SUPT6H, SUPT16H, SV2C, SYCP2, SYT6, SYCP1, SYTL3, SYTL5, TAF2, TARDBP, TBCID3G, TBC1D8B, TBC1D26, TBC1D29, TBCEL, TBK1, TBP, TBPL1, TBR1, TBX, TCEB3, TCF3, TCF4, TCF7L2, TCFL5, TCF12, TCPIIL2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGS1, THAP7, THAP12, THOC2, TIAL1, TIAM2, TIMM50, TLK2, TM4SF20, TM6SF1, TMEM27, TMEM77, TMEM156, TMEM194A, TMF1, TMPRSS6, TNFRSFIOA, TNFRSFIOB, TNFRSF8, TNK2, TNKS, TNKS2, TOMIL, TOMIL2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTF1, TTLL5, TTLL9, TTN, TTPAL, TTR, TUSC3, TXNDC10, UBE3A, UCK1, UGTIA1, UHRFIBP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VTIA, VTIIB, VWA3B, WDFY2, WDR16, WDR17, WDR26, WDR44, WDR67, WDTC1, WRN, WRNIP1, WT1, WWC3, XBP1, XRN1, XRN2, XX—FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPM1, ZFYVE1, ZFX ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include AICF, A4GALT, AAR2, ABAT, ABCA11P, ZNF721, ABCA5, ABHD10, ABHD13, ABHD2, ABHD6, AC0000120.3, KRIT1, AC004076.1, ZNF772, AC004076.9, ZNF772, AC004223.3, RAD51D, AC004381.6, AC006486.1, ERF, AC0007390.5, AC0007780.1, PRKAR1A, AC0007998.2, IN080C, AC0009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDDIA, AC073610.3, ARF3, AC074091.1, GPN, AC079447.1, LIPT1, AC092587.1, AC079594.2, TRIM59, AC091060.1, C18orf21, AC092143.3, MC1R, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHD1L, ACADM, ACAP3, ACKR2, RP11-141M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADHIB, ADIPOR1, ADNP, ADPRH, AGBL5, AGPAT1, AGPAT3, AGR2, AGTR1, AHDC1, AHI1, AHNAK, AIFM1, AIFM3, AIMP2, AK4, AKAP1, AKNAD1, CLCC1, AKRIA1, AKT1, AKTIS1, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDHIB1, ALDH3A1, ALDH3B2, ALDOA, ALKBH2, ALPL, AMD1, AMICA1, AMN1, AMOTL2, AMYJB, AMY2B, ANAPC10, ANAPCI1, ANAPC15, ANG, RNASE4, AL163636.2, ANGEL2, ANGPTL1, ANKMY1, ANKRDI1, ANKRD28, ANKRD46, ANKRD9, ANKS3, ANKS3, RP11-127I20.7, ANKS6, ANKZF1, ANPEP, ANXA11, ANXA2, ANXA8L2, AL603965.1, AOC3, AP000304.12, CRYZL, AP000311.1, CRYZL, AP000893.2, RAB30, AP001267.5, ATP5MG, AP002495.2, AP003175.1, OR2AT4, AP003419.1, CLCF1, AP005263.1, ANKRD12, AP006621.5, AP006621.1, AP1G1, AP3M1, AP3M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARID1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATP5FIA, ATP5G2, ATP5J, ATP5MD, ATP5PF, ATP6AP2, ATP6VOB, ATP6V1C1, ATP6VID, ATP7B, ATXN1, ATXN1L, IST1, ATXA3, ATXA7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCSIL, BDH1, BDKRB2, AL355102.2, BEST1, BEST3, BEX4, BHLHB9, BID, BIN3, BIRC2, BIVM, BIVM-ERCC5, BIVM, BLCAP, BLK, BLOC1S1, RP11-644F5.10, BLOC1S6, AC090527.2, BLOC1S6, RP11-96020.4, BLVRA, BMF, BOLA1, BORCS8-MEF2B, BORCS8, BRCA1, BRD1, BRDT, BRINP3, BROX BTBD10, BTBD3, BTBD9, BTD, BTF3L4, BTNL9, BUB1B-PAK6, PAK6, BUB3, C10orf68, C11orf1, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, C1D, C1GALT1, C1QB, C1QTNF1, C1S, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CA5BP1, CASB, CABYR, CALCA, CALCOCO1, CALCOCO2, CALM1, CALM3, CALML4, RP11-315D16.2, CALN, CALU, CANT1, CANX, CAP1, CAPN12, CAPS2, CARD8, CARHSP1, CARNS1, CASC1, CASP3, CASP7, CBFA2T2, CBS, CBY1, CCBL1, CCBL2, RBMXL1, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB1IP1, CCNC, CCND3, CCNG1, CCP110, CCR9, CCT7, CCT8, CD151, CDID, CD200, CD22, CD226, CD276, CD36, CD59, CDC26, CDC42, CDC42SE1, CDC42SE2, CDHR3, CDK10, CDK16, CDK4, CDKAL1, CDKL3, CTD-2410N18.4, CDKNIA, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHDIL, CHD8, CHFR, ZNF605, CHIA, CHID1, CHL1, CHM, CHMPIA, CHMP3, RNF103-CHMP3, CHRNA2, CIDEC, CIRBP, CITED1, CKLF-CMTM1, CMTM1, CKMT1B, CLDN12, CTB-13L3.1, CLDND1, AC021660.3, CLDND1, CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COAl, COASY, COCH, COL8A1, COLCA1, COLEC11, COAMD3-BMI1, BMI1, COPS5, COPS7B, COQ8A, CORO6, COTL1, COX14, RP4-60503.4, COX7A2, COX7A2L, COX7B2, CPA4, CPA5, CPEB1, CPNE1, AL109827.1, RBM12, CPNE1, RP1-309K20.6, RBM12, CPNE3, CPSF3L, CPTIC, CREB3L2, CREM, CRP, CRYZ, CS, AC073896.1, CS, RP11-977G19.10, CSAD, CSDE, CSF2RA, CSGALNACT, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNNDl, CTPS2, CTSB, CTSL, CTTN, CUL2, CUL9, CWC5, CXorf40B, CYB561A3, CYBC1, CYLD, CYP11A1, CYP2R1, CYP4B1, CYP4F22, DAG1, DAGLB, KDELR2, DARS, DBNL, DCAF1l, DCAF8, PEX19, DCLREIC, DCTD, DCTN1, DCTN4, DCUN1D2, DDR1, DDX11, DDX19B, AC012184.2, DDX19B, RP11-529K10.3, DDX25, DDX39B, ATP6V1G2-DDX39B, SNORD84, DDX42, DDX60L, DEDD, DEDD2, DEFA1, DEFA1B, DEFA1B, DEFA3, DENNDIC, DENND2A, DENND4B, DET, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKNV, DMTF1, DMTN, DNAJC14, DNAJC19, DNAL1, DNASElL1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RPL1-500M8.7, DZIP1L, E2F6, ECHDC1, ECSIT, ECT2, EDC3, EDEM1, EDEM2, MMP24-AS1, RP4-61404.11, EEF1AKNMT, EEF1D, EFEMP1, EFHC1, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOV1, ELOVL7, ELP1, ELP6, EML3, EMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERLIN2, ERMARD, ERRFI1, ESR2, RP11-544I20.2, ESRRA, ESRRB, ESRRG, ETFA, ETFRF1, ETV1, ETV4, ETV7, EVAIA, EVC2, EVX1, EXD2, EXO5, EXOC1, EXOC2, FAAP24, FABP6, FADS1, FADS2, FAHD2B, FAM107B, FAM111A, FAMIIB, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANC1, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLR1, FOXP1, FOXK1, FOAM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT1l, GALNT14, GALNT6, GAPVD1, GARNL3, GAS2L3, GAS8, GATA1, GATA2, GATA4, GBA, GCNT1, GDPD2, GDPD5, GEMIN7, MARK4, GEMIN8, GGA3, GGACT, AL356966.1, GGPS1, GHRL, GID8, GIGYF2, GIMAP8, GIPC1, GJB1, GJB6, GLBIL, GLI1, GLT8D1, GMFG, GMPR2, GNAI2, GNAQ, GNB1, GNB2, GNE, GNG2, GNGT2, GNPDA1, GNPDA2, GOLGA3, CHFR, GOLGA4, GOLPH3L, GOLT1B, GPBPL, GPER, GPR16, GPR141, EPDR1, GPR155, GPR161, GPR56, GPR63, GPR75-ASB3, ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCY1A3, GUCYIB3, GUK1, GULP1, GYPC, GYS1, GZF1, HAGH, HA02, HAPLN3, HAVCR1, HAX1, HBG2, AC104389.4, HBG2, AC104389.4, HBE1, HBG2, AC104389.4, HBE1, OR51B5, HBG2, HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX HOXA3, HOXB3, HOXB3, HOXB4, HOXC4, HOXD3, HOXD3, HOXD4, HPCAL1, HPS4, HPS5, HRH1, HS3ST3A1, HSH2D, HSP90AA1, HSPD1, HTT, HUWE1, HYOU1, IAH1, ICA1L, ICAM2, ICE2, ICK, IDH2, IDH3G, IDS, IFI27, IFI44, IFT20, IFT22, IFT88, IGF2, INS-IGF2, IGF2BP3, IGFBP6, IKBKAP, IKBKB, IL11, IL18BP, IL18RAP, ILIRAP, IL1RL1, IL18R1, IL1RN, IL32, IL4I1, NUP62, AC011452.1, IL4I1, NUP62, CTC-326K19.6, IL6ST, ILVBL, IMMP1L, IMPDH1, INCA1, ING1, INIP, INPP1, INPP5J, INPP5K, INSIG2, INTS11, INTS12, INTS14, IP6K2, IP6K3, IPO11, LRRC70, IQCE, IQGAP3, IRAK4, IRF3, IRF5, IRF6, ISG20, IST1, ISYNA1, ITFG2, ITGBIBP1, ITGB7, ITIH4, RP5-966M1.6, ITPRIPL1, JADE1, JAK2, JARID2, JDP2, KANK1, KANK1, RP11-31F19.1, KANK2, KANSLIL, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDMIB, KDM4A, AL451062.3, KHNYN, KIAA0040, KIAA0125, KIAA0196, KIAA0226L, PPPIR2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC1, APOPT1, AL139300.1, KLC4, KLHDC4, KLHDC8A, KLHL13, KLHL18, KLHL2, KLHL24, KLHL7, KLKI1, KLK2, KLK5, KLK6, KLK7, KNOP1, KRBA2, AC135178.2, KRBA2, RP11-849F2.7, KRIT1, KRT15, KRT8, KTN1, KXD1, KYAT3, RBMXL1, KYNU, L3MBTL1, LACC1, LARGE, LARP4, LARP7, LAT2, LBHD1, LCA5, LCA5L, LCTL, LEPROTL1, LGALS8, LGALS9C, LGMN, LHFPL2, LIG4, LIMCH1, LIMK2, LIMS2, LINC00921, ZNF263, LIPF, LLGL2, LMAN2L, LMCD1, LMF1, RP11-161M6.2, LMO1, LMO3, LOXHD1, LPAR1, LPAR2, LPAR4, LPAR5, LPAR6, LPHN1, LPIN2, LPIN3, LPP, LRFN5, LRIF1, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYG1, LYL1, LYPD4, LYPD6B, LYRM1, LYRM5, LYSMD4, MACC1, MADIL1, MADIL1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASP1, MATK, MATR3, MATR3, SNHG4, MB, MBD5, MBNL1, MBOAT7, MCC, MCFD2, MCM9, MCOLN3, MCRS1, MDC1, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGFJO, MEIl, MEIS2, MELK, MET, METTL13, METTL23, MFF, MFN2, MFSD2A, MGST3, MIB2, MICAL1, MICAL3, MICOSO, NBL1, MICOS10-NBL1, MID1, MINA, MINOSI-NBL1, MINOS1, MIOS, MIPOL1, MIS12, MKLN1, MKNK1, MKNK1, MOB3C, MLF2, MLH1, MMP17, MOBP, MOCS1, MOGS, MOK, MORF4L1, MPC1, MPC2, MPG, MP1, MPP1, MPP2, MPPE1, MPST, MRAS, MRO, MROH1, MROH7-TTC4, MROH7, MRPL14, MRPL24, MRPL33, BABAM2, MRPL33, BRE, MRPL47, MRPL48, MRPL55, MRRF, MRTFA, MRTFB, MRVI1, MS4A1, MS4A15, MS4A3, MS4A6E, MS4A7, MS4A14, MSANTD3, MSANTD4, MSH5, MSH5-SAPCDl, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMR10, MTRF1, MTRR, MTUS2, MUTYH, MVK, MX1, MX2, MYH10, MYL12A, MYB, MYD88, MYL5, MYLIP, MYNN, MYO15A, MYOIB, MYOM2, MZF1, N4BP2L2, NAA60, NAB1, NAEl, NAGK, NAP1L1, NAPIL4, NAPG, NARFL, NARG2, NAT1, NAT10, NBPF11, WI2-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFV1, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOLO, NOL8, NONO, NPAS1, NPIPA8, RP11-1212A22.1, NPIPB3, NPIPB4, NPIPB9, NPL, NPM1, NPPA, NQO2, NR1H3, NR2C2, NR2F2, NR4A1, NRDC, NREP, NRF1, NRG4, NRIP1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RP11-197N18.2, OLA1, OPRL1, OPTN, OR2H1, ORAI2, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARPH1, PARVG, PASK, PAX6, PBRM1, PBXIPl, PCBP3, PCBP4, AC115284.1, PCBP4, RP11-155D18.14, RP11-155D18.12, PCGF3, PCGF5, PCNP, PCSK9, PDCD10, PDCD6, AHRR, PDDC1, PDGFRB, PDIA6, PDIKIL, PDLIM7, PDP1, PDPK1, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4 KB, PIAS2, PICALM, PIF1, PIGN, PIGO, PIGT, PIK3CD, PILRB, STAG3L5P-PVRIG2P-PILRB, PIP5K1B, PIR, PISD, PIWIL4, FUT4, PKD2, PKIA, PKIG, PKM, PKN2, PLA1A, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP, AKTIS1, PNMT, PNPLA4, PNPLA8, PNPO, PNRC1, POCIB, POFUT1, POLB, POLD1, POLH, POL1, POLL, POLRIB, POM121, POM121C, AC006014.7, POM121C, AC211429.1, POMC, POMT1, POP1, PORCN, POU5F1, PSORSIC3, PPARD, PPARG, PPHLN, PPIL3, PPIL4, PPM1A, PPM1B, AC013717.1, PPPICB, PPP1R11, PPPIR13L, PPPIR26, PPPIR9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAG1, PRMT2, PRMT5, PRMT7, PROM1, PRPS1, PRPSAP2, PRR14L, PRR15L, PRR5, PRR5-ARHGAP8, PRR5L, PRR7, PRRC2B, PRRT4, PRSS50, PRSS45, PRSS44, PRUNE, PRUNE1, PSEN1, PSMA2, PSMF1, PSORSIC1, PSPH, PSRC1, PTBP3, PTHLH, PTK2, PTPDC1, PTPRM, PUF60, PUM2, PUS1, PUS10, PXA, PXYLP1, PYCR1, QRICH1, R3HCC1L, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAP1, RAD17, RAD51L3-RFFL, RAD51D, RAD52, RAE1, RAI14, RAI2, RALBP1, RAN, RANGAP1, RAP1A, RAP1B, RAPIGAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7, AP002373.1, RBM7, RP11-212D19.4, RBMS2, RBMYIE, RBP1, RBPMS, RBSN, RCBTB2, RCC1, RCC1, SNHG3, RCCD1, RECQL, RELL2, REPIN1, AC073111.3, REPIN1, ZNF775, RER1, RERE, RFWD3, RFX3, RGL2, RGMB, RGS11, RGS3, RGS5, AL592435.1, RHBDD1, RHNO1, TULP3, RHOC, AL603832.3, RHOC, RP11-426L16.10, RHOH, RIC8B, RIMKLB, RIN1, RIPK2, RIT1, RLIM, RNASE4, ANG, AL163636.6, RNASEK, RNASEK-C17orf49, RNFI1, RNF123, RNF13, RNF14, RNF185, RNF216, RNF24, RNF32, RNF34, RNF38, RNF4, RNF44, RNH1, RNMT, RNPS1, RO60, ROPN1, ROPNIB, ROR2, RP1-102H19.8, C6orf163, RP1-283E3.8, CDK11A, RP11-120M18.2,PRKARIA, RP11-133K10.2, PAK6, RP11-164J13.1, CAPN3, RP11-21JJ8.1, ANKRD12, RP11-322E10.6, INO80C, RP11-337C18.10, CHD1L, RP11-432B6.3, TRIM59, RP11-468E2.4, IRF9, RP11-484M3.5, UPKIB, RP11-517H2.6, CCR6, RP11-613M10.9, SLC25A51, RP11-659G9.3, RAB30, RP11-691N7.6, CTNND1, RP11-849H4.2, RP11-896J10.3, NKX2-1, RP11-96020.4, SQRDL, RP11-986E7.7, SERPINA3, RP4-769N13.6, GPRASP1, RP4-769N13.6, GPRASP2, RP4-798P15.3, SEC16B, RP5-1021I20.4, ZNF410, RP6-109B7.3, FLJ27365, RPE, RPH3AL, RPL15, RPL17, RPL17-C18orf32, RPL17, RPL23A, RPL36, HSDI1BIL, RPP38, RPS20, RPS27A, RPS3A, RPS6KA3, RPS6KC1, RPS6KL1, RPUSD1, RRAGD, RRAS2, RRBP1, RSLID1, RSRC2, RSRP1, RUBCNL, RUNXIT1, RUVBL2, RWDD1, RWDD4, S100A13, AL162258.1, S100A13, RP1-178F15.5, S100A16, S100A4, S100A3, S100A6, S100PBP, SAA1, SACMIL, SAMD4B, SARIA, SARAF, SARNP, RP11-762I7.5, SCAMP5, SCAP, SCAPER, SCFD1, SCGB3A2, SCIN, SCML1, SCNNID, SC02, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-Sep, 2-Sep, SERGEF, AC055860.1, SERP1, SERPINA1, SERPINA5, SERPINB6, SERPING1, SERPINH1, SERTAD3, SETD5, SFMBT1, AC096887.1, SFTPA1, SFTPA2, SFXN2, SGCD, SGCE, SGK3, SGK3, C8orf44, SH2B1, SH2D6, SH3BP1, Z83844.3, SH3BP2, SH3BP5, SH3D19, SH3YL1, SHC1, SHISA5, SHMT1, SHMT2, SHOC2, SHROOM1, SIGLEC5, SIGLEC14, SIL1, SIN3A, SIRT2, SIRT6, SKP1, STAT4, AC104109.3, SLAIN1, SLCI0A3, SLC12A9, SLC14A1, SLC16A6, SLCIA2, SLCIA6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCOIA2, SLCO1C1, SLCO2B1, SLFNI1, SLFN12, SLFNL1, SLMO1, SL™, SLU7, SMAD2, SMAP2, SMARCA2, SMARCEl, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPD1, SNRPD2, SNRPN, SNRPN, SNURF, SNUPN, SNXI1, SNX16, SNX17, SOAT1, SOHLH2, CCDC169-SOHLH2, CCDC169, SORBS1, SORBS2, SOX5, SP2, SPART, SPATA20, SPATA21, SPATS2, SPATS2L, SPDYE2, SPECC1, SPECC1L, SPECC1L-ADORA2A, SPECC1L-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX2IP, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RP11-463D19.2, STEAP2, STEAP3, STIL, STK25, STK33, STK38L, STK40, STMN1, STON1, STONI-GTF2A1L, STRAP, STRBP, STRC, AC011330.5, STRC, CATSPER2, STRC, CATSPER2, AC011330.5, STRC, STRCP1, STT3A, STX16-NPEPL1, NPEPL1, STX5, STX6, STX8, STXBP6, STYK1, SULTIA1, SULTIA2, SUMF2, SUN1, SUN2, SUN2, DNAL4, SUOX, SUPT6H, SUV39H2, SV2B, SYBU, SYNCRIP, SYNJ2, SYT1, SYTL4, TAB2, TACC1, TADA2B, TAFIC, TAF6, AC073842.2, TAF6, RP11-506M12.1, TAF9, TAGLN, TANK, TAPSAR1, PSMB9, TAPT1, TATDN1, TAZ, TBC1D1, TBCID12, HELLS, TBCID15, TBCID3H, TBCID3G, TBCID5, TBCID5, SATB1, TBCA, TBCEL, TBCEL, AP000646.1, TBL1XR1, TBP, TBX5, TBXAS1, TCAF1, TCEA2, TCEAL4, TCEAL8, TCEAL9, TCEANC, TCEB1, TCF19, TCF25, TCF4, TCP1, TCPIOL, AP000275.65, TCPI1, TCPIIL2, TCTN1, TDG, TDP1, TDRD7, TEAD2, TECR, TENC1, TENT4A, TEX264, TEX30, TEX37, TFDP1, TFDP2, TFEB, TFG, TFP1, TF, TFP1, TGIF1, THAP6, THBS3, THOC5, THRAP3, THUMPD3, TIAL1, TIMM9, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSSIID, TMPRSS5, TMSB15B, TMTC4, TMUB2, TMX2-CTNND1, RP11-691N7.6, CTNND1, TNFAIP2, TNFAIP8L2, SCNM1, TNFRSF10C, TNFRSF9, TNFRSF8, TNFSF12-TNFSF3, TNFSF2, TNFSF3, TNFSF12-TNFSF3, TNFSF3, TNIP1, TNK2, TNNT, TNRC18, TNS3, TOB2, TOMIL1, TOPIMT, TOP3B, TOX2, TP53, RP11-199F1.2, TP53I1, TP53INP2, TPCN, TPM3P9, AC022137.3, TPT1, TRA2B, TRAF2, TRAF3, TRAPPC12, TRAPPC3, TREH, TREX, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMTIOB, TRMT2B, TRMT2B-AS1, TRNT, TRO, TROVE2, TRPS1, TRPT1, TSC2, TSGA10, TSPAN14, TSPAN3, TSPAN4, TSPAN5, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD, TYK2, U2AF1, UBA2, UBA52, UBAP2, UBE2D2, UBE2D3, UBE2E3, UBE21, UBE2J2, UBE3A, UBL7, UBXN11, UBXN7, UGDH, UGGT1, UGP2, UMAD1, AC007161.3, UNC45A, UQCC1, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VIL1, VIPR1, VPS29, VPS37C, VPS8, VPS9D1, VRK2, VWA1, VWA5A, WARS, WASF1, WASHC5, WBP5, WDHD1, WDPCP, WDR37, WDR53, WDR6, WDR72, WDR74, WDR81, WDR86, WDYHV1, WFDC3, WHSC1, WIPF1, WSCD2, WWP2, XAGEA, XAGEIB, XKR9, XPNPEP1, XRCC3, XRN2, XXYL T1, YIFIA, YIFIB, YIPF1, YIPF5, YPEL5, YWHAB, YWHAZ, YYJAP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFAND5, ZFAND6, ZFP1, ZFP62, ZFX, ZFYVE16, ZFYVE19, ZFYVE20, ZFYVE27, ZHX2, AC016405.1, ZHX3, ZIK1, ZIM2, PEG3, ZKSCAN1, ZKSCAN3, ZKSCAN8, ZMAT3, ZMAT5, ZMIZ2, ZMYM6, ZMYND11, ZNF10, AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC0008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158I13.2, ZNF512B, ZNF512B, SAMD10, ZNF521, ZNF532, ZNF544, AC020915.5, ZNF544, CTD-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOAM1 gene.

Exemplary genes that may be modulated by the compounds of Formula (I) or (II) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgcgaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucagcg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugagcg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugcguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuauu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacuge (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugagcg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugaguc (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgcgccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacage (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguagcug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugageg (SEQ ID NO: 957), AUGgugagcu (SEQ ID NO: 958), AUGgugaggc (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugcgau (SEQ ID NO: 966), AUGgugcgug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguuaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgcgccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacccg (SEQ ID NO: 1173), CAGguacccu (SEQ ID NO: 1174), CAGguaccgc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacugc (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucagcg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccugc (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugcg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugagcg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugcgcc (SEQ ID NO: 1376), CAGgugcgcg (SEQ ID NO: 1377), CAGgugcgga (SEQ ID NO: 1378), CAGgugcggu (SEQ ID NO: 1379), CAGgugcgua (SEQ ID NO: 1380), CAGgugcguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcugc (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggcca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucccg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugcgua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CCAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugcgcg (SEQ ID NO: 1589), CCGgugggcg (SEQ ID NO: 1590), CCGguugguc (SEQ ID NO: 1591), CCUguaaaug (SEQ ID NO: 1592), CCUguaaauu (SEQ ID NO: 1593), CCUguaagaa (SEQ ID NO: 1594), CCUguaagac (SEQ ID NO: 1595), CCUguaagag (SEQ ID NO: 1596), CCUguaagca (SEQ ID NO: 1597), CCUguaagcg (SEQ ID NO: 1598), CCUguaagga (SEQ ID NO: 1599), CCUguaaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugagaa (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguaagag (SEQ ID NO: 1622), CGGgcaggca (SEQ ID NO: 1623), CGGguaagcc (SEQ ID NO: 1624), CGGguaagcu (SEQ ID NO: 1625), CGGguaaguu (SEQ ID NO: 1626), CGGguaauuc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggcc (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugccuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaaug (SEQ ID NO: 1661), CUAguaagcg (SEQ ID NO: 1662), CUAguaagcu (SEQ ID NO: 1663), CUAguaagua (SEQ ID NO: 1664), CUAguaaguc (SEQ ID NO: 1665), CUAguaagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CUGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGgucccgc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugcgug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacagc (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguaccga (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GAGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauucc (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucaggc (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugagcg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugcggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugagcg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugcgua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GUAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaagcg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagege (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugaggc (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UACguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UCCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccueg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagcc (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacggc (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UGGgccccgg (SEQ ID NO: 2820), UGGguaaaau (SEQ ID NO: 2821), UGGguaaagc (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguaagaa (SEQ ID NO: 2832), UGGguaagac (SEQ ID NO: 2833), UGGguaagag (SEQ ID NO: 2834), UGGguaagau (SEQ ID NO: 2835), UGGguaagca (SEQ ID NO: 2836), UGGguaagcc (SEQ ID NO: 2837), UGGguaagcu (SEQ ID NO: 2838), UGGguaaggg (SEQ ID NO: 2839), UGGguaaggu (SEQ ID NO: 2840), UGGguaagua (SEQ ID NO: 2841), UGGguaaguc (SEQ ID NO: 2842), UGGguaagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggeg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054).

Additional exemplary gene sequences and splice site sequences (e.g., 5′ splice site sequences) include AAGgcaagau (SEQ ID NO: 96), AUGguaugug (SEQ ID NO: 937), GGGgugaggc (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgcgagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GAGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGgugguge (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggcgc (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgcgggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugagge (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugageg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgcgcgcg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugagcg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugegug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgegguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugcgug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggcg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacace (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugagcg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgcgggaa (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguagugc (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucuggc (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugcguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugcgga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgcggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugcg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugagcg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgccgguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugcguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaagc (SEQ ID NO: 3523), CUGguaaaag (SEQ ID NO: 3524), CCGgugcgga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucagcg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaagcg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaagcg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugegge (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaagcg (SEQ ID NO: 3684), AAGguccgcg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggcg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacucg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugcgcg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugagcg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGguggugc (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccuge (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaagcg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugcggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugaggc (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaacc (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugcgua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgcgagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugaugc (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGguguggc (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacugc (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggcg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucguce (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugagge (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGguccgcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucucgc (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugcg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugcguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgcccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugcggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggcg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggece (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagagc (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugcgcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaaauu (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugagcg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugagcg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugaggc (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggcg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550). Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaagcg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugeguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugcggc (SEQ ID NO: 4673), AGGguacuga (SEQ ID NO: 4674), AGAguaagcg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggcgg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUgucegcc (SEQ ID NO: 4836), AAGgugcgga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugeguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgcagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugcgcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugcguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggagc (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugagcg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgcauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugcgcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugcgcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuauuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I) or (II) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, Ul snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C1/C2. Human genes encoding hnRNPs include HNRNPA0, HNRNPA1, HNRNPA1L1, HNRNPA1L2, HNRNPA3, HNRNPA2B1, HNRNPAB, HNRNPBI, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I) or (II) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I) or (II) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I) or (II) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7% 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I) or (II) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I) or (II). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I) or (II) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I) or (II) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I) or (II) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I) or (II), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I) or (II), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I) or (II) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I) or (II) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MDS).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I) or (II) or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I) or (II), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I) or (II) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I) or (II) or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I) or (II) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I) or (II) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I) or (II) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I) or (II) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I) or (II) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I) or (II) increases expression of the haploinsufficient gene locus. In an embodiment, a compound of Formula (I) or (II) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I) or (II), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I) or (II) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I) or (II), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I) or (II) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I) or (II), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I) or (II) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I) or (II) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or (II) or a pharmaceutically acceptable salt thereof, e.g., as described herein, has a cell permeability measurement (Papp) of <2×10⁻⁶ cm s⁻¹. In an embodiment, a compound of Formula (I) or (II) or a pharmaceutically acceptable salt thereof, e.g., as described herein, has a cell permeability measurement (Papp) of between 2-6×10⁻⁶ cm s⁻¹ In an embodiment, a compound of Formula (I) or (II) or a pharmaceutically acceptable salt thereof, e.g., as described herein, has a cell permeability measurement (Papp) of Papp greater than 6×10⁻⁶ cm s⁻¹. In an embodiment, a compound of Formula (I) or (II) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or (II) or a pharmaceutically acceptable salt thereof, e.g., as described herein, has a cell efflux ratio of less than 1.5. In an embodiment, a compound of Formula (I) or (II) or a pharmaceutically acceptable salt thereof, e.g., as described herein, has a cell efflux ratio of between 1.5 and 5. In an embodiment, a compound of Formula (I) or (II) or a pharmaceutically acceptable salt thereof, e.g., as described herein, has a cell efflux ratio greater than 5. In an embodiment, a compound of Formula (I) or (II) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, modulates the expression of a target protein (e.g., HTT or MYB) in a reference cell or sample. In an embodiment, a compound of Formula (I) or (II) or a pharmaceutically acceptable salt thereof, e.g., as described herein, increases the expression of a target protein (e.g., HTT or MYB) in a reference cell or sample. In an embodiment, a compound of Formula (I) or (II) or a pharmaceutically acceptable salt thereof, e.g., as described herein, decreases the expression of a target protein (e.g., HTT or MYB) in a reference cell or sample. The effect of an exemplary compound of Formula (I) or (II) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I) or (II) or a pharmaceutically acceptable salt thereof, e.g., as described herein, has a protein abundance response less than 100 nM. In an embodiment, a compound of Formula (I) or (II) or a pharmaceutically acceptable salt thereof, e.g., as described herein, has a protein abundance response between 100-1000 nM. In an embodiment, a compound of Formula (I) or (II) or a pharmaceutically acceptable salt thereof, e.g., as described herein, has a protein abundance response greater than 1000 nM. In an embodiment, a compound of Formula (I) or (II) or a pharmaceutically acceptable salt thereof, e.g., as described herein, has a protein abundance response greater than 10 uM. In an embodiment, a compound of Formula (I) or (II) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, modulates the viability of a target cell in a subject or sample. In an embodiment, a compound of Formula (I) or (II) or a pharmaceutically acceptable salt thereof, e.g., as described herein, increases the viability of a target cell in a subject or sample. In an embodiment, a compound of Formula (I) or (II) or a pharmaceutically acceptable salt thereof, e.g., as described herein, decreases the viability of a target cell in a subject or sample. In an embodiment, a compound of Formula (I) or (II) or a pharmaceutically acceptable salt thereof, e.g., as described herein, does not impact the viability of a cell (e.g., is non-toxic) in a subject or sample. The effect an exemplary compound of Formula (I) or (II) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I) or (II) or a pharmaceutically acceptable salt thereof, e.g., as described herein, is tolerated by a target cell at a concentration of less than 100 nM. In an embodiment, a compound of Formula (I) or (II) or a pharmaceutically acceptable salt thereof, e.g., as described herein, is tolerated by a target cell at a concentration of between 100-1000 nM. In an embodiment, a compound of Formula (I) or (II) or a pharmaceutically acceptable salt thereof, e.g., as described herein, is tolerated by a target cell at a concentration of greater than 1000 nM. In an embodiment, a compound of Formula (I) or (II) or a pharmaceutically acceptable salt thereof, e.g., as described herein, is tolerated by a target cell at a concentration of greater than 10 uM. In some embodiments, a compound of Formula (I) or (II) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,bmin}) may be defined as the ratio of unbound brain-free compound concentration to unbound plasma concentration. It is calculated using the following equation

$${K}_{p , uu , brain} = \frac{{f}_{u , brain} \times {C}_{brain}}{{f}_{u , plasma} \times {C}_{plasma}}$$

C_{brain} and Cₚₗₐₛₘₐ represent the total concentrations in brain and plasma, respectively. In this assay, the f_{u,brain} and f_{u,plasma} may be the unbound fraction of the compound in brain and plasma, respectively. Both f_{u,brain} and f_{u,plasma} may be determined in vitro via equilibrium dialysis. In an embodiment, a compound of Formula (I) or (II) or a pharmaceutically acceptable salt thereof, e.g., as described herein, has a Kp value of greater than 5. In an embodiment, a compound of Formula (I) or (II) or a pharmaceutically acceptable salt thereof, e.g., as described herein, has a Kp value between 1 and 5. In an embodiment, a compound of Formula (I) or (II) or a pharmaceutically acceptable salt thereof, e.g., as described herein, has a Kp value between 0.2-1.

In an embodiment, a compound of Formula (I) or (II) or a pharmaceutically acceptable salt thereof, e.g., as described herein, has a Kp value of less than 0.2. In an embodiment, a compound of Formula (I) or (II) or a pharmaceutically acceptable salt thereof, e.g., as described herein, has a Kpuu value of greater than 2.5. In an embodiment, a compound of Formula (I) or (II) or a pharmaceutically acceptable salt thereof, e.g., as described herein, has a Kpuu value between 0.5-2.5. In an embodiment, a compound of Formula (I) or (II) or a pharmaceutically acceptable salt thereof, e.g., as described herein, has a Kpuu value between 0.1-0.5. In an embodiment, a compound of Formula (I) or (II) or a pharmaceutically acceptable salt thereof, e.g., as described herein, has a Kpuu value of less than 0.1. In an embodiment, a compound of Formula (I) or (II) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I) or (II) or a pharmaceutically acceptable salt thereof, e.g., as described herein, exhibits selectivity for HTT, e.g., an HTT-related nucleic acid sequence. In an embodiment, a compound of Formula (I) or (II) or a pharmaceutically acceptable salt thereof, e.g., as described herein, exhibits selectivity for SMN2, e.g., an SMN2-related nucleic acid sequence. In an embodiment, a compound of Formula (I) or (II) or a pharmaceutically acceptable salt thereof, e.g., as described herein, exhibits selectivity for Target C, e.g., a Target C-related nucleic acid sequence. In an embodiment, a compound of Formula (I) or (II) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I) or (II) or a pharmaceutically acceptable salt thereof, e.g., as described herein, has a 3-fold greater selectivity for HTT over MYB. In an embodiment, a compound of Formula (I) or (II) or a pharmaceutically acceptable salt thereof, e.g., as described herein, has a 3-fold greater selectivity for MYB over HTT. In an embodiment, a compound of Formula (I) or (II) or a pharmaceutically acceptable salt thereof, e.g., as described herein, has a 10-fold greater selectivity for HTT over MYB. In an embodiment, a compound of Formula (I) or (II) or a pharmaceutically acceptable salt thereof, e.g., as described herein, has a 10-fold greater selectivity for MYB over HTT. In an embodiment, a compound of Formula (I) or (II) or a pharmaceutically acceptable salt thereof, e.g., as described herein, has a 3-fold greater selectivity for HTT over SMN2. In an embodiment, a compound of Formula (I) or (II) or a pharmaceutically acceptable salt thereof, e.g., as described herein, has a 3-fold greater selectivity for SMN2 over HTT. In an embodiment, a compound of Formula (I) or (II) or a pharmaceutically acceptable salt thereof, e.g., as described herein, has a 10-fold greater selectivity for HTT over SMN2. In an embodiment, a compound of Formula (I) or (II) or a pharmaceutically acceptable salt thereof, e.g., as described herein, has a 10-fold greater selectivity for SMN2 over HTT. In an embodiment, a compound of Formula (I) or (II) or a pharmaceutically acceptable salt thereof, e.g., as described herein, has a 3-fold greater selectivity for MYB over SMN2. In an embodiment, a compound of Formula (I) or (II) or a pharmaceutically acceptable salt thereof, e.g., as described herein, has a 3-fold greater selectivity for SMN2 over MYB. In an embodiment, a compound of Formula (I) or (II) or a pharmaceutically acceptable salt thereof, e.g., as described herein, has a 10-fold greater selectivity for MYB over SMN2. In an embodiment, a compound of Formula (I) or (II) or a pharmaceutically acceptable salt thereof, e.g., as described herein, has a 10-fold greater selectivity for SMN2 over MYB. In an embodiment, a compound of Formula (I) or (II) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I) or (II), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (6) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 m, 120 Å, 40° C.) operating in ESI(+) ionization mode; flow rate=1.2 Ml/min. Mobile phase=0.05% TFA in water or CH₃CN; or on Shimadzu-2020EV using column: Poroshell HPH—C18 (C18, Ø4.6×50 mm, 3 m, 120 Å, 40° C.) operating in ESI(+) ionization mode; flow rate=1.2 Ml/min. Mobile phase A: Water/5Mm NH₄HCO₃, Mobile phase B: CH₃CN.) Analytical chiral HPLC: Analytical chiral HPLC was performed on a Agilent 1260 using column: CHIRALPAK IG-3, CHIRALPAK IC-3 or CH IRALPAK OJ-3, with flow rate=1.2 Ml/min. Mobile phase=MTBE(DEA):EtOH=50:50).

Analytical chiral HPLC: Analytical chiral HPLC was performed on a Agilent 1260 using column: CHLRALPAK IG-3, CHIRALPAK IC-3 or CHIRALPAK OJ-3, with flow rate=1.2 Ml/min. Mobile phase=MTBE(DEA):EtOH=50:50).

Preparative HPLC purification: prep-HPLC purification was performed using one of the following HPLC conditions:

Condition 1: Shimadzu, Column: Xbridge Prep OBD C18 Column, 30A 150 mm 5 μm; Mobile Phase A: water (10 mmol/L NH₄HCO₃) Mobile Phase B: acetonitrile; Flow rate: 60 Ml/min; Gradient 1: 3 B to 3 B in 2 min; Gradient 2: 5% B to 35% B in 6 min; Gradient 3: 3 B to 33 B in 6 min; Gradient 4: 5% B up to 45% in 6 min; Gradient 5: 3% B to 23% B in 6 min; Gradient 6: 10% B to 60% B in 8 min; Gradient 7: 5 B to 45 B in 10 min; Gradient 8: 10% B up to 47% B in 10 min; Gradient 9: 10% B up to 50% B in 8 min; Gradient 9: 5% B to 35% B in 8 min; Gradient 10: 10% B to 48% B in 10 min; Gradient 11: 20% B to 52% B in 8 min; Gradient 12: 20% B to 50% B in 6 min; Gradient 13: 20% B to 43% B in 8 min; Gradient 14: 15% B to 45% B in 8 min; Gradient 14: 10% B to 55% B in 8 min; Gradient 15: 5% B to 38% B in 10 min; Gradient 16: 10% B to 35% B in 8 min; Gradient 17: 5% B to 42% B in 8 min; Gradient 18: 5% B to 30% B in 8 min; Gradient 18: 5% B to 40% B in 8 min; Gradient 19: 5% B to 45% B in 8 min; Gradient 21: 5% B to 37% B in 8 min; Gradient 22: 5% B to 65% B in 8 min; Gradient 23: 10% B to 65% B in 8 min; Gradient 24: 5% B to 50% B in 8 min.

Condition 2: Column: Xselect CSH OBD Column 30*150 mm 5 μm, n; Mobile Phase A: water (10 mmol/L NH₄HCO₃); Mobile Phase B: acetonitrile; Flow rate: 60 Ml/min; Gradient 1: 10 B to 55 B in 8 min; Gradient 2: 5 B to 50 B in 8 min; Gradient 3: 10 B to 60 B in 10 min; Gradient 4: 10 B to 40 B in 8 min; Gradient 5: 5 B to 65 B in 8 min; Gradient 6: 3% B to 63% B in 6 min; Gradient 7: 10% B to 52% B in 8 min; Gradient 8: 5% B to 37% B in 8 min; Gradient 9: 10% B to 38% B in 8 min; Gradient 10: 3% B to 75% B in 8 min; Gradient 11: 10% B to 42% B in 8 min; Gradient 12: 15% B to 40% B in 10 min; Gradient 13: 10% B to 60% B in 8 min; Gradient 14: 5% B to 35% B in 8 min; Gradient 15: 15% B to 36% B in 8 min.

Condition 3: Column: EP-C18M 10 μm 120A; Mobile Phase A: water (1 mmol/L HCl); Mobile Phase B: acetonitrile; Flow rate: 100 Ml/min; Gradient: 40% B to 70% B in 35 min.

Condition 4: Column: Poroshell HPH—C18, 3.0*50 mm, 2.7 um; Mobile Phase A: water (5 Mm NH₄HCO₃); Mobile Phase B: acetonitrile; Flow rate: 1.2 Ml/min; Gradient 1:10% B to 95% B in 1.2 min, hold 0.5 min.

Condition 5: Column: X Select CSH OBD 30×150 mm 5 μm; Mobile phase A: water (0.1% formic acid); Mobile phase B: acetonitrile; Gradient 1: 3% phase B up to 18% in 6 min.

Condition 6: Column: X Select CSH OBD 30×150 mm 5 μm; Mobile phase A: water (0.05% HCl); Mobile phase B: acetonitrile; Flow rate: 60 Ml/min; Gradient 1: 3% phase B up to 3% in 2 min; Gradient 2: 3% B to 18% B in 8 min.

Condition 7: Column: X Select CSH OBD 30×150 mm 5 μm; Mobile phase A: water (0.05% formic acid); Mobile phase B: acetonitrile; Flow rate: 60 Ml/min; Gradient 1: 3% phase B up to 20% in 8 min.

Condition 8: Column: YMC-Actus Triart C18, 30 mm×150 mm, 5 μm; Mobile phase A: water (0.05% HCl); Mobile phase B: acetonitrile; Gradient 1: 5% B to 35% B in 8 min.

Condition 9: Column: YMC-Actus Triart C18, 30 mm×150 mm, 5 μm; Mobile phase A: water (10 mmol/L NH₄HCO₃); Mobile phase B: acetonitrile; Flow rate: 60 Ml/min Gradient 1: 10% B to 70% B in 8 min; Gradient 2: 15% B to 55% B in 8 min; Gradient 3: 5% B to 65% B in 8 min; Gradient 4: 5% B to 45% B in 8 min; Gradient 5:15% B to 45% B in 10 min; Gradient 6: 15% B to 70% B in 8 min; Gradient 7: 5% B to 50% B in 8 min; Gradient 8: 15% B to 50% B in 8 min; Gradient 9: 20% B to 44% B in 10 min.

Condition 10: XBridge Prep OBD Column 19×150 mm 8 μm; Mobile Phase A: Water (0.05% NH₃·H₂O), Mobile Phase B: ACN; Flow rate: 20 mL/min; Gradient 1: 20% B to 50% B in 8 min; Gradient 2: 25% B to 55% B in 8 min; Gradient 3: 35% B to 65% B in 14 min; Gradient 4: 10% B to 45% B in 12 min; Gradient 5: 24% B to 54% B in 8 min; Gradient 6: 45% B to 65% B in 14 min; Gradient 7: 55% B to 82% B in 9 min; Gradient 8: 20% B to 50% B in 7 min; Gradient 9: 56% B to 76% B in 8 min; Gradient 10: 24% B to 47% B in 8 min; Gradient 11: 30% B to 60% B in 8 min; Gradient 12: 15% B to 45% B in 8 min; Gradient 13: 20% B to 45% B in 8 min; Gradient 14: 50% B to 70% B in 12 min; Gradient 15: 50% B to 80% B in 8 min; Gradient 16: 40% B to 75% B in 7 min.

Condition 11: Column: YMC-Actus Triart C18 ExRS, 30×250 mm, 5 m; Mobile Phase A: Water (10 mmol/L NH₄HCO₃+0.1% NH₃·H₂O); Mobile Phase B: Acetonitrile; Flow rate: 35 mL/min; Gradient 1: 33% B to 74% B in 7 min

Condition 12: Column: Welch Ultimate XB—C18, 50×250 mm, 10 μm; Mobile Phase A: Water (0.1% NH₃·H₂O), Mobile Phase B: Acetonitrile; Gradient 1: 5% B to 45% B in 10 min; Gradient 2: 5% B to 35% B in 10 min; Gradient 3: 10% B to 55% B in 10 min;

Condition 13: SunFire Prep Column 19×150 mm, 10 μm, Mobile Phase A: Water (0.05% NH₄OH), Mobile Phase B: Acetonitrile, Gradient 1: 30% B to 50% B in 7 min;

Condition 14: SunFire Prep Column 19×150 mm, 10 μm, Mobile Phase A: Water (0.05% TFA), Mobile Phase B: Acetonitrile, Gradient 1: 21% B to 35% B in 14 min; Gradient 2: 10% B to 20% B in 7 min

Condition 15: Welch Ultimate XB—C18, 50×250 mm, 10 μm; Mobile Phase A: Water (0.1% TFA), Mobile Phase B: Acetonitrile; Gradient 1: 10% B to 50% B in 12 min; Gradient 2: 30% B to 60% B in 10 min; Gradient 3: 10% B to 45% B in 12 min; Gradient 4: 35% B to 70% B in 10 min;

Condition 16: Welch Ultimate XB—C18, 50×250 mm, 10 μm; Mobile Phase A: Water (0.1% NaHCO₃), Mobile Phase B: Acetonitrile; Gradient 1: 35% B to 75% B in 10 min.

Condition 17: Column: Xselect C18, 19×150 mm, 5 m; Mobile Phase A: 0.1% TFA, Mobile Phase B: Acetonitrile; Flow rate: 20 mL/min; Gradient 1: 5% B to 40% B in 7 min.

Condition 18: Column, C18 silica gel, XBridge, 19×150 mm; Mobile Phase A: Water (0.05% HCl), Mobile Phase B: Acetonitrile, Gradient 1: 20% B to 30% B in 14 min Preparative chiral HPLC: purification by chiral HPLC was performed on a Gilson-GX 281 using column: CHIRALPAK IG-3, CHIRALPAK IC-3 or CHIRALPAK OJ-3.

Condition 1: Column: CHIRALPAK IG, 3×25 cm, 5 μm; Mobile Phase A: MTBE (0.1% DEA), Mobile Phase B: ethanol; Flow rate: 20 Ml/min; Gradient 1: 50 B to 50 B in 18 min.

Condition 2: Column: CHIRAL ART Cellulose-SC, 3×25 cm, 5 m; Mobile Phase A: MTBE (0.1% DEA)-HPLC, Mobile Phase B: MeOH—-HPLC; Flow rate: 35 mL/min; Gradient 1: to 30% B isocratic in 22 min, Gradient 2: 50% B isocratic in 36 min.

Condition 3: Column: CHIRAL ART Cellulose-SC, 3×25 cm, 5 m; Mobile Phase A: HEX:DCM=3:1 (0.2% DEA), Mobile Phase B: Ethanol; Flow rate: 35 mL/min; Gradient 1: 50% B to 50% B in 22 min;

Condition 4: Column: CHIRALPAK IH, 3×25 cm, 5 m; Mobile Phase A: MTBE(2 mM NH₃-MEOH), Mobile Phase B: IPA:DCM=1:1; Flow rate: 35 mL/min; Gradient 1: 25% B to 25% B in 12 min

Condition 5: Column, CHIRAL ART Cellulose-SC, 3×25 cm, 5 m; Mobile Phase A: MTBE (0.5% 2M NH₃-MeOH), Mobile Phase B: IPA:ACN=2: 1; Flow rate: 35 mL/min; Gradient 1: 30% B isocratic

Condition 6: Column: CHIRAL ART Cellulose-SB, 3×25 cm, 5 m; Mobile Phase A: Hex (0.2% IPAmine), Mobile Phase B: EtOH:DCM=1:1; Flow rate: 35 mL/min; Gradient 1: 20% B isocratic

Condition 7: Column: CHIRALPAK IA, 2*25 cm, 5 m; Mobile Phase A: Hex:DCM=5: 1, Mobile Phase B: EtOH (0.1% 2M NH₃-MeOH); Flow rate: 25 mL/min; Gradient 1: 45% B isocratic in 18 min;

Condition 8: Column: CHIRALPAK IF, 3×25 cm, 5 m; Mobile Phase A: HEX:DCM=3:1-HPLC, Mobile Phase B: EtOH (0.1% DEA)-HPLC; Flow rate: 35 mL/min; Gradient 1: 50% B isocratic in 40 min

Condition 9: Column: CHIRALPAK IC, 2×25 cm, 5 m; Mobile Phase A: Hex:DCM=1:1-HPLC, Mobile Phase B: EtOH (0.1% DEA)-HPLC; Flow rate: 23 mL/min; Gradient 1: 50% B isocratic in 90 min.

Condition 10: Column: CHIRAL ART Cellulose-SZ, 3×25 cm, 5 m; Mobile Phase A: Hex:DCM=1:1-HPLC, Mobile Phase B: EtOH (0.1% DEA)-HPLC; Flow rate: 30 mL/min; Gradient 1: 50% B isocratic in 12 min.

Condition 11: Column: CHIRAL ART Amylose-C NEO, 3×25 cm, 5 am; Mobile Phase A: EtOH (0.5% 2M NH3-MeOH)-HPLC, Mobile Phase B: EtOH (0.5% 2M NH₃-MeOH)—HPLC; Flow rate: 28 mL/min; Gradient 1: 50% B isocratic in 15 min.

Condition 12: Column: CHIRALPAK IG, 3×25 cm, 5 m; Mobile Phase A: HEX:DCM=3:1-HPLC, Mobile Phase B: EtOH (0.5% 2M NH₃-MeOH)-HPLC; Flow rate: 30 mL/min; Gradient 1: 50% B isocratic in 16 min.

Reverse flash chromatography: purification by reverse flash chromatography was performed using one of the following conditions:

Condition 1: Column, C18; Mobile phase: MeOH in water; Gradient 1, 10% to 50% in 10 min; Detector, UV 254 nm.

Condition 2: Column, silica gel; Mobile phase: MeOH in water; Gradient 1: 10% to 50% in 10 min; Detector, UV 254 nm.

Condition 3: Column, C18 silica gel; mobile phase A: Water (0.1% NH₃H₂O), Mobile Phase B: ACN; Gradient 1: 30% B to 80% B gradient in 12 min; Gradient 2: 20% B to 60% B in 12 min; Gradient 3: 10% B to 80% B in 15 min; Gradient 4: 5% B to 40% B in 12 min; Gradient 5: 20% B to 50% B in 12 min; Gradient 6: 30% B to 60% B in 10 min; Gradient 7: 20% B to 50% B in 7 min; Gradient 8: 20% B to 70% B in 12 min; Gradient 9: 10% B to 100% B in 15 min; Gradient 10: 5% B to 35% B in 10 min.

Condition 4: Column: ACE 5AQ, 21.2×150 mm, 5 m; Mobile Phase A: Water (0.1% TFA), Mobile Phase B: Acetonitrile/Methanol (1:1); Flow rate: 20 mL/min; Gradient: 5% B to 40% B in 8 min

Condition 5: Column: C18 silica gel; Mobile Phase A: Water (0.1% FA), Mobile Phase B: Acetonitrile. Gradient 1: 30% B to 70% B in 12 min; Gradient 2: 30% B to 80% B in 12 min; Gradient 3: 20% B to 60% B in 10 min; Gradient 4: 24% B to 40% B in 7 min; Gradient 5: 40% B to 80% B in 10 min; Gradient 6: 10% B to 50% B in 10 min; Gradient 7: 5% B to 35% B in 10 min.

Condition 6: Column: C18 silica gel; Mobile Phase A: Water (0.1% TFA), Mobile Phase B: Acetonitrile; Gradient 1: 20% B to 50% B in 10 min; Gradient 2: 10% B to 35% B in 10 min; Gradient 3: 20% B to 70% B in 12 min.

Condition 7: Column: C18 silica gel; Mobile Phase A: Water (0.5% NH₃) Mobile Phase B: Acetonitrile; Gradient 1: 40% B to 70% B in 10 min; Gradient 2: 30% B to 50% B in 12 min; Gradient 3: 30% B to 60% B in 12 min;

Condition 8: Column: C18 silica gel, XBridge, 19×150 mm; Mobile Phase A: Water (0.05% NH₃·H₂O), Mobile Phase B: Acetonitrile, Gradient 1: 20% B to 50% B in 7 min;

Condition 9: Column: C18 silica gel, XBridge, 19×150 mm; Mobile Phase A: Water (0.05% TFA), Mobile Phase B: Acetonitrile, Gradient 1: 20% B to 50% B in 7 min.

Condition 10: Column: C18 silica gel; Mobile Phase A: Water (10 mmol/L NH₄HCO₃), Mobile Phase B: Acetonitrile; Gradient 1: 10% B to 50% B in 10 min.

Thin Layer chromatography: purification by thin layer chromatography was performed using one of the following conditions:

Condition 1: Column: YMC-Actus Triart C18 ExRS, 30×250 mm, 5 m; Mobile Phase A: Water (10 mmol/L NH₄HCO₃+0.1% NH₃·H₂O), Mobile Phase B: Acetonitrile; Flow rate: 35 mL/min; Gradient 1: 15% B to 70% B in 8 min; Gradient 2: 16% B to 64% B in 8 min

### General Synthetic Schemes

Compounds of the present disclosure may be prepared using a synthetic protocol illustrated in one of Schemes A, B, or C.

### General Synthetic Schemes

Compounds of the present disclosure may be prepared using a synthetic protocol illustrated in one of Schemes A, B, or C.

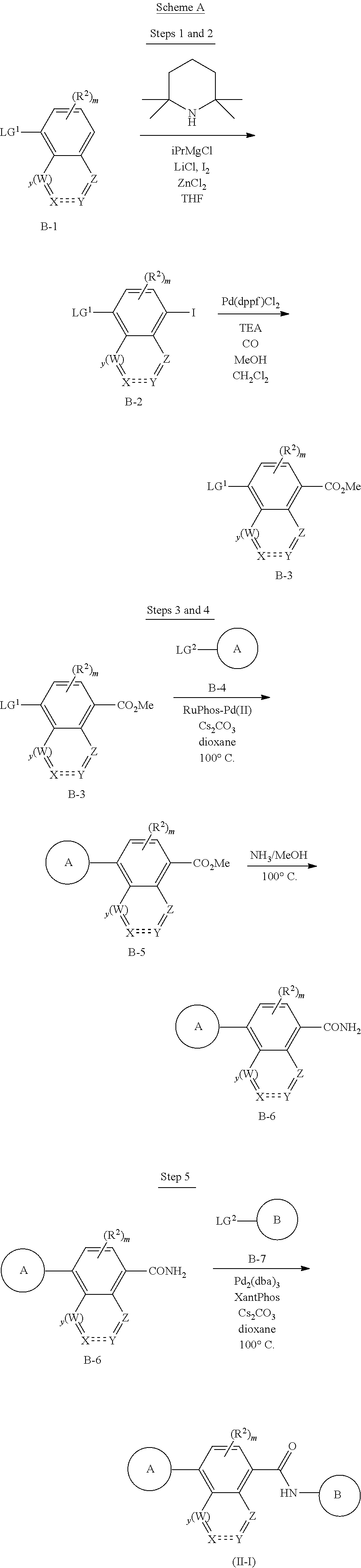

Scheme A. An exemplary method of preparing a compound of Formula (I); wherein A, B, W, X, Y, Z, R², and m are as defined herein; and LG¹, LG², and LG³ are each independently a leaving group (e.g., halo, —B(OR¹²)₂). In some embodiments of the application, y is 0.

An exemplary method of preparing a compound described herein, e.g., a compound of Formula (II-I) is provided in Scheme A. In Step 1, B-2 is prepared by treating B-1 with a mixture of 2,2,6,6-tetramethylpiperidine, isopropylmagnesium chloride (iPrMgCl), lithium chloride (LiCl), iodine (I₂), and zinc chloride (ZnCl₂) in tetrahydrofuran (THF), or with a similar combination of reagents or solvent. In Step 2, B-3 is prepared by incubating B2 with 1,1′-bis(diphenylphosphino)ferrocene)palladium(II) dichloride (Pd(dppf)Cl₂), carbon monoxide (CO), and triethylamine (TEA), in a mixture of methanol (MeOH) and dichloromethane (CH₂Cl₂) or a similar mixture of solvents. Alternative catalysts to Pd(dppf)Cl₂ may also be used, such as a suitable palladium catalyst, and/or using alternative reagents sufficient to provide B-3.

In Step 3, B-5 is prepared by incubating B-3 with B-4 in the presence of RuPhos-Pd(II) (e.g., RuPhos-Pd(II)-G2 or RuPhos-Pd(II)-G3), and cesium carbonate (Cs₂CO₃) or a similar reagent. Step 3 may also be carried out using an alternative catalyst to RuPhos-Pd(II), such as another ruthenium catalyst. The reaction may be conducted in dioxane or a similar solvent, at 100° C. or a temperature sufficient to provide B-5. B-5 is then converted to B-6 by treatment with a mixture of ammonia and methanol, at 100° C. or a temperature sufficient to provide B-6.

B-6 and B-7 are coupled to provide a compound of Formula (II-I) in Step 5. This coupling reaction may be conducted in the presence of tris(dibenzylideneacetone)dipalladium(0) (Pd₂(dba)₃, XantPhos, and cesium carbonate or a suitable alternative. Step 5 may also be carried out using an alternative catalyst to Pd₂(dba)₃, such as another palladium catalyst, and/or an alternative ligand to XantPhos (e.g., a different phosphine ligand). The reaction may be conducted in dioxane or a similar solvent, at 100° C. or a temperature sufficient to provide the compound of Formula (II-I). Each starting material and/or intermediate in Scheme B may be protected and deprotected using standard protecting group methods. In addition, purification and characterization of each intermediate as well as the final compound of Formula (II) may be afforded by any accepted procedure.

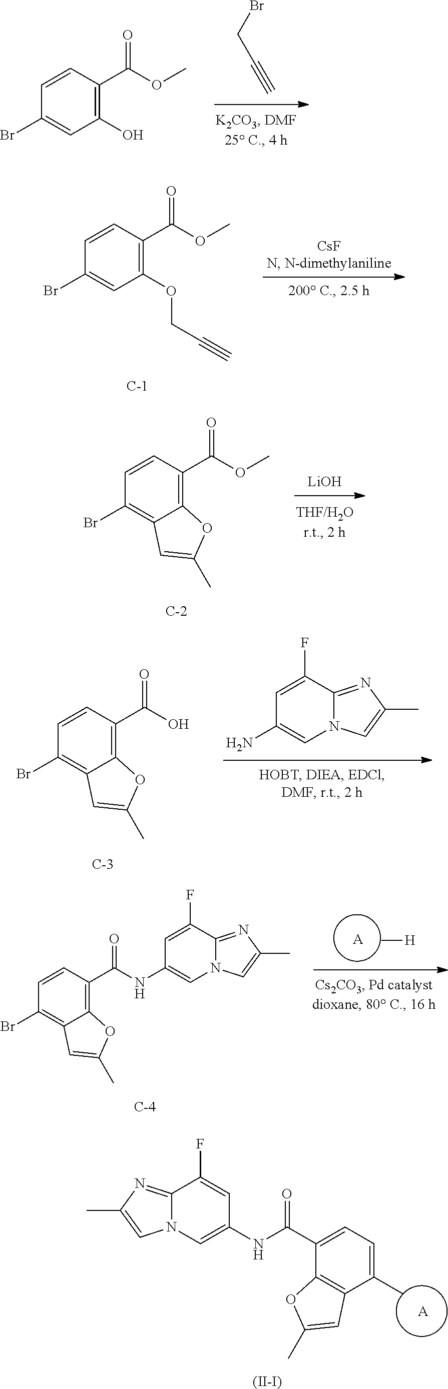

Scheme B. An exemplary method of preparing a compound of Formula (I); wherein A is as defined herein.

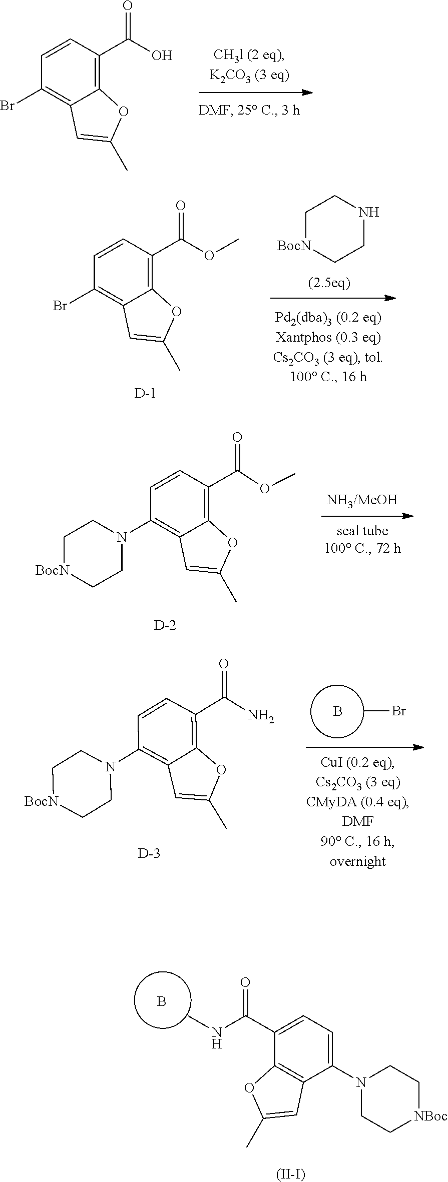

Scheme C. An exemplary method of preparing a compound of Formula (I); wherein B is as defined herein.

Exemplary protocols for the synthesis of compounds in Tables 1 and 2, e.g., Compounds 1-287, can be found in Examples 1-41 in WO 2021/174165, which is incorporated herein by reference in its entirety.

### Example 42: Synthesis of Compound 303

### Synthesis of Intermediate C1

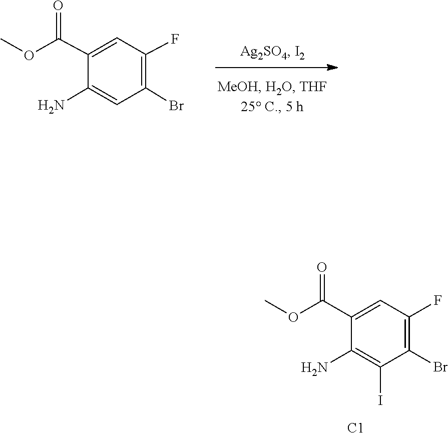

Methyl 2-amino-4-bromo-5-fluorobenzoate (100.0 mg, 0.403 mmol, 1.0 equiv), methanol (1 mL), water (1 mL), 1,1-dioxo-1-sulfonylidenedisilver (201.1 mg, 0.645 mmol, 1.6 equiv), iodine (163.7 mg, 0.645 mmol, 1.6 equiv) and tetrahydrofuran (1 mL) were combined at 25° C. The resulting mixture was stirred for 5 h at 25° C., then diluted with water (20 mL) and extracted with ethyl acetate (2×20 mL). The organic layers were combined, dried by Na₂SO₄, filtered and the filtrate concentrated under reduced pressure to give a residue. The residue was purified by silica gel column chromatography, eluted with PE/EA (5:1) to afford methyl 2-amino-4-bromo-5-fluoro-3-iodobenzoate (70 mg, 45.5%) as a solid. LCMS (ES, m/z): 373 [M+H]⁺.

### Synthesis of Intermediate C2

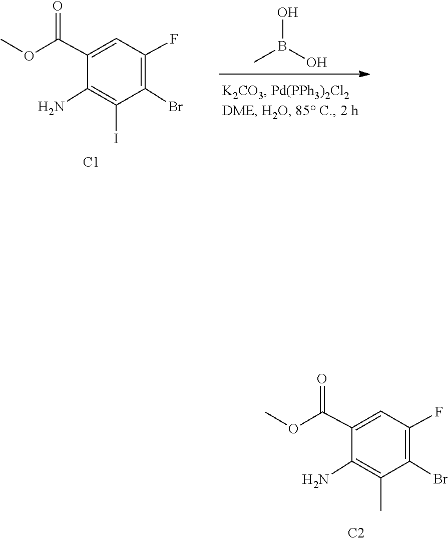

To a mixture of methyl 2-amino-4-bromo-5-fluoro-3-iodobenzoate (2.8 g, 7.488 mmol, 1.0 equiv) and methylboronic acid (2.6 g, 44.928 mmol, 6.0 equiv) in DME (40 mL) and H₂O (10 mL) was added K₂CO₃ (2.07 g, 14.976 mmol, 2.0 equiv) and Pd(PPh₃)₂Cl₂ (0.5 g, 0.749 mmol, 0.1 equiv). The reaction mixture was stirred for 6 h at 80° C. under a nitrogen atmosphere, then concentrated under reduced pressure to give a residue. The residue was purified by silica gel column chromatography, eluted with PE/EA (5:1) to afford methyl 2-amino-4-bromo-5-fluoro-3-methylbenzoate (1.2 g, 46%) as a solid. LCMS (ES, m/z): 262 [M+H]⁺.

### Synthesis of Intermediate C3

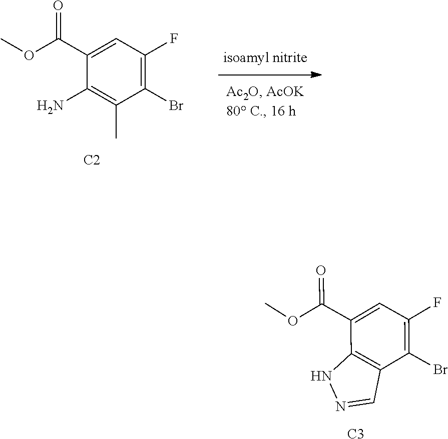

Methyl 2-amino-4-bromo-5-fluoro-3-methylbenzoate (1.2 g, 4.579 mmol, 1.0 equiv) and Ac₂O (0.6 g, 5.953 mmol, 1.3 equiv) were combined at 25° C. The resulting mixture was stirred for 1 h at 25° C. To the reaction mixture was added potassium acetate (0.13 g, 1.374 mmol, 0.3 equiv) and isoamyl nitrite (1.1 g, 10.074 mmol, 2.2 equiv). The resulting mixture was stirred for an additional 2 h at 80° C., then quenched with water (100 mL) at 25° C. and extracted with CH₂C12 (2×100 mL). The organic layers were combined, dried by Na₂SO₄, filtered, and the filtrate concentrated under reduced pressure to give a residue. The residue was purified by silica gel column chromatography, eluted with PE/EA (1:1) to afford methyl 4-bromo-5-fluoro-1H-indazole-7-carboxylate (0.18 g, 12%) as a solid. LCMS (ES, m/z): 273 [M+H]⁺.

### Synthesis of Intermediate C4

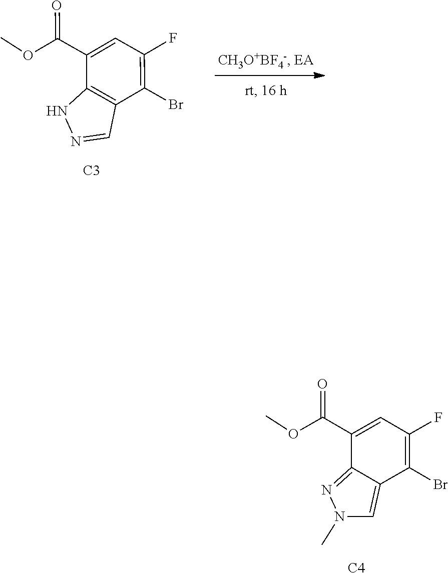

Methyl 4-bromo-5-fluoro-1H-indazole-7-carboxylate (50.0 mg, 0.183 mmol, 1.0 equiv), ethyl acetate (2 mL), and boron trifluoride trimethyloxidanium fluoride (108.3 mg, 0.732 mmol, 4.0 equiv) were combined at room temperature. The resulting mixture was stirred for 16 h at room temperature, then quenched with water and extracted with ethyl acetate (2×5 mL). The organic layers were combined, dried by Na₂SO₄, filtered, and the filtrate concentrated under reduced pressure to give a residue. The residue was purified by silica gel column chromatography, eluted with PE/EA (1:1) to afford methyl 4-bromo-5-fluoro-2-methylindazole-7-carboxylate (51 mg, 87%) as a solid. LCMS (ES, m/z): 287 [M+H]+.

### Synthesis of Intermediate C5

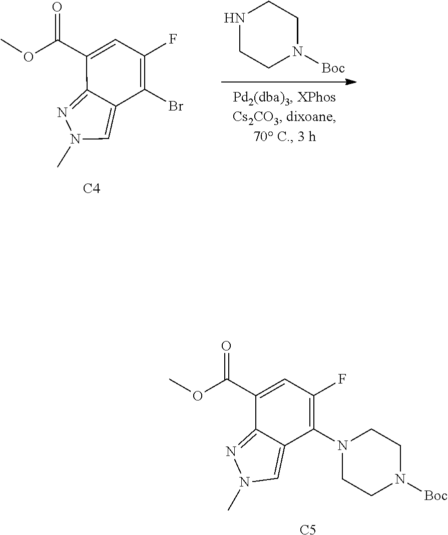

Methyl 4-bromo-5-fluoro-2-methylindazole-7-carboxylate (180.0 mg, 0.627 mmol, 1.0 equiv), Cs₂CO₃ (409.8 mg, 1.254 mmol, 2.0 equiv), tert-butyl piperazine-1-carboxylate (233.5 mg, 1.254 mmol, 2.0 equiv), XPhos (59.7 mg, 0.125 mmol, 0.2 equiv), Pd₂(dba)₃ (57.4 mg, 0.063 mmol, 0.1 equiv) and dioxane (5 mL) were combined at room temperature. The resulting mixture was stirred for 3 h at 70° C. under nitrogen atmosphere, then concentrated under reduced pressure to give a residue. The residue was purified by silica gel column chromatography, eluted with CH₂Cl₂/MeOH (12:1) to afford methyl 4-[4-(tert-butoxycarbonyl)piperazin-1-yl]-5-fluoro-2-methylindazole-7-carboxylate (230 mg, 93%) as a solid. LCMS (ES, m/z): 393 [M+H]⁺.

### Synthesis of Intermediate C6

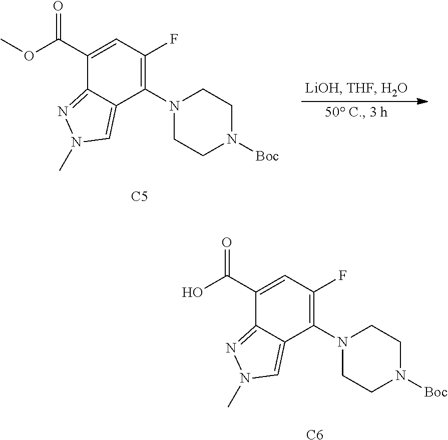

Methyl 4-[4-(tert-butoxycarbonyl)piperazin-1-yl]-5-fluoro-2-methylindazole-7-carboxylate (230.0 mg, 0.586 mmol, 1.0 equiv), tetrahydrofuran (2 mL), water (2 mL) and LiOH (140.3 mg, 5.860 mmol, 10.0 equiv) were combined at room temperature. The resulting mixture was stirred for 3 h at 50° C., then diluted with water (30 mL), acidified to pH 5 with HCl (aq.), and extracted with ethyl acetate (2×50 mL). The organic layers were combined, dried by Na₂SO₄, filtered, and the filtrate concentrated under reduced pressure to afford 4-[4-(tert-butoxycarbonyl)piperazin-1-yl]-5-fluoro-2-methylindazole-7-carboxylic acid (150 mg, 62%) as a solid. LCMS (ES, m/z): 385 [M+H]⁺.

### Synthesis of Intermediate C7

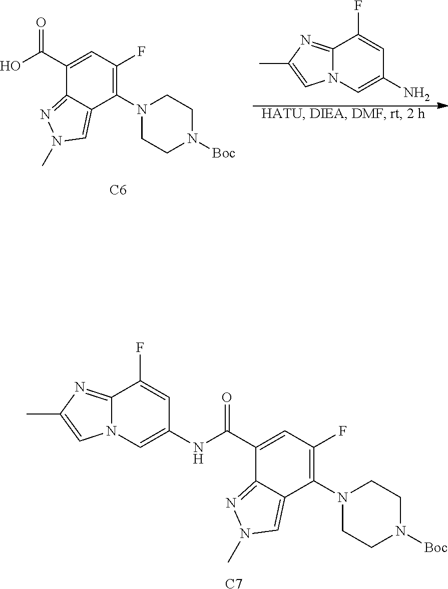

A mixture of 4-[4-(tert-butoxycarbonyl)piperazin-1-yl]-5-fluoro-2-methylindazole-7-carboxylic acid (70.0 mg, 0.185 mmol, 1.0 equiv), 8-fluoro-2-methylimidazo[1,2-a]pyridin-6-amine (39.7 mg, 0.240 mmol, 1.3 equiv), HATU (140.6 mg, 0.370 mmol, 2.0 equiv), DMF (3 mL), and DIEA (95.6 mg, 0.740 mmol, 4.0 equiv) was stirred for 2 h at room temperature, then diluted with water (10 mL) at room temperature and extracted with ethyl acetate (2×10 mL). The organic layers were combined, dried over Na₂SO₄, filtered, and the filtrate concentrated under reduced pressure to afford tert-butyl 4-[5-fluoro-7-({8-fluoro-2-methylimidazo[1,2-a]pyridin-6-yl}carbamoyl)-2-methylindazol-4-yl]piperazine-1-carboxylate (75 mg, 71%) as a solid. LCMS (ES, m/z): 526 [M+H]⁺.

Synthesis of Compound 303

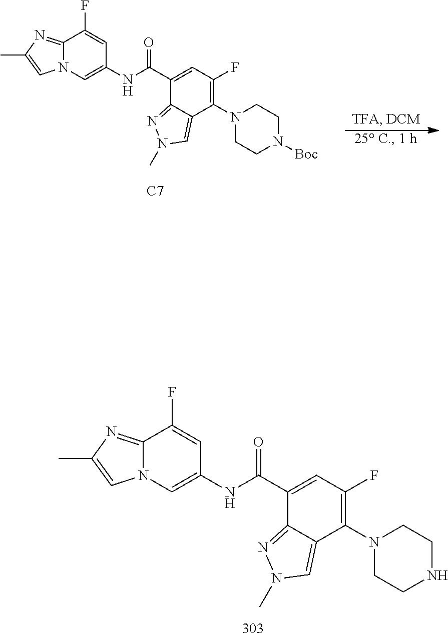

A mixture of tert-butyl 4-[5-fluoro-7-({8-fluoro-2-methylimidazo[1,2-a]pyridin-6-yl}carbamoyl)-2-methylindazol-4-yl]piperazine-1-carboxylate (70.0 mg, 0.133 mmol, 1.0 equiv), DCM (1 mL), and TFA (1 mL) was stirred for 1 h at room temperature, then concentrated under reduced pressure to give a residue. The residue was purified by Prep-HPLC (Condition 1, Gradient 1) to afford 5-fluoro-N-{8-fluoro-2-methylimidazo[1,2-a]pyridin-6-yl}-2-methyl-4-(piperazin-1-yl)indazole-7-carboxamide (32.1 mg, 56.37%) as a solid. LCMS (ES, m/z): 426 [M+H]⁺. ¹H NMR (300 MHz, DMSO-d₆) δ 11.11 (m, 1H), 9.37 (d, J=1.6 Hz, 1H), 8.91 (s, 3H), 8.07 (d, J=2.6 Hz, 1H), 7.86 (d, J=14.1 Hz, 1H), 7.67 (d, J=12.1 Hz, 1H), 4.34 (s, 3H), 3.68 (d, J=5.1 Hz, 4H), 3.32 (s, 4H), 2.42 (s, 3H)

### Example 43: Synthesis of Compound 290

### Synthesis of Intermediate C8

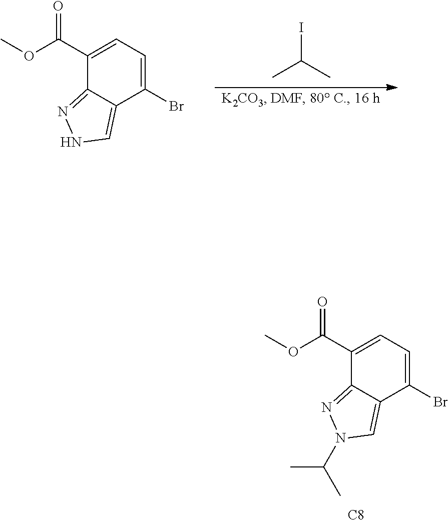

To a stirred mixture of methyl 4-bromo-2H-indazole-7-carboxylate (2.0 g, 7.841 mmol, 1.0 equiv) and K₂CO₃ (1625.4 mg, 11.761 mmol, 1.5 equiv) in DMF (20 mL) was added 2-iodopropane (2.0 g, 11.761 mmol, 1.5 equiv) dropwise at room temperature. The resulting mixture was stirred for 16 h at 80° C., then cooled to room temperature, diluted with water (60 mL), and extracted with ethyl acetate (2×50 mL). The organic layers were combined, washed with water (1×100 mL) and brine (1×100 mL), dried over anhydrous Na₂SO₄, and filtered. After filtration, the filtrate was concentrated under reduced pressure to give a residue. The residue was purified by silica gel column chromatography, eluted with PE/EA (3:1) to afford methyl 4-bromo-2-isopropylindazole-7-carboxylate (450 mg, 19.31%) as an oil. LCMS (ES, m/z): 297 [M+H]⁺. ¹H NMR (400 MHz, DMSO-d₆) δ 8.66 (s, 1H), 7.83 (d, J=7.6 Hz, 1H), 7.41 (d, J=7.6 Hz, 1H), 4.93 (p, J=6.7 Hz, 1H), 3.89 (s, 3H), 1.59 (d, J=6.7 Hz, 6H).

### Synthesis of Intermediate C9

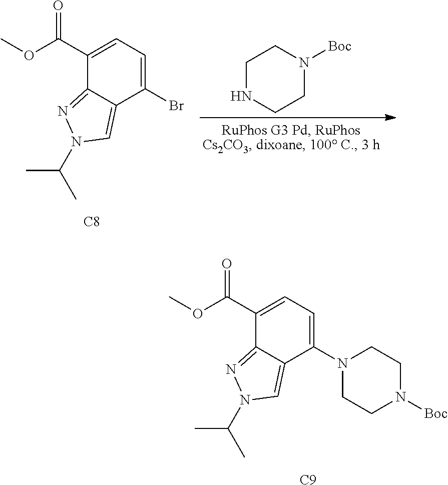

To a mixture of methyl 4-bromo-2-isopropylindazole-7-carboxylate (200.0 mg, 0.673 mmol, 1.0 equiv), tert-butyl piperazine-1-carboxylate (250.7 mg, 1.346 mmol, 2.0 equiv), and Cs₂CO₃ (659.9 mg, 2.019 mmol, 3.0 equiv) in dioxane (2 mL) was added RuPhos (62.8 mg, 0.135 mmol, 0.2 equiv) and RuPhos G3 Pd (56.3 mg, 0.067 mmol, 0.1 equiv). After stirring for 3 h at 100° C. under a nitrogen atmosphere, the resulting mixture was concentrated under reduced pressure to give a residue. The residue was purified by silica gel column chromatography, eluted with PE/EA (1:1) to afford methyl 4-[4-(tert-butoxycarbonyl)piperazin-1-yl]-2-isopropylindazole-7-carboxylate (250 mg, 92%) as a solid. LCMS (ES, m/z): 403 [M+H]⁺.

### Synthesis of Intermediate C10

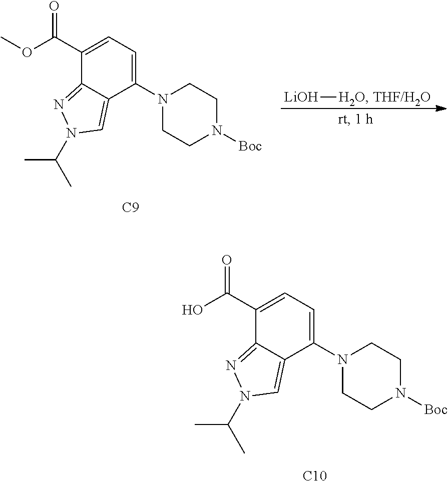

A mixture of methyl 4-[4-(tert-butoxycarbonyl) piperazin-1-yl]-2-isopropylindazole-7-carboxylate (250 mg, 0.621 mmol, 1.00 equiv), tetrahydrofuran (1.2 mL), water (1.2 mL), and LiOH—H₂O (39.1 mg, 0.931 mmol, 1.5 equiv) was stirred for 1 h at room temperature. The reaction mixture was acidified to pH 6 with HCl (1 N, aq.) in ice-water bath, then extracted with ethyl acetate (3×3 mL). The organic layers were combined, dried over anhydrous Na₂SO₄, and filtered. After filtration, the filtrate was concentrated under reduced pressure to afford 4-[4-(tert-butoxycarbonyl)piperazin-1-yl]-2-isopropylindazole-7-carboxylic acid (100 mg, 41%) as a solid. LCMS (ES, m/z): 389 [M+H]⁺.

### Synthesis of Intermediate C11

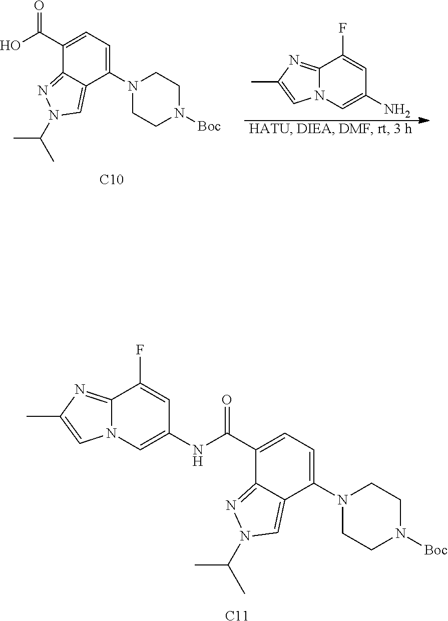

A mixture of 4-[4-(tert-butoxycarbonyl)piperazin-1-yl]-2-isopropylindazole-7-carboxylic acid (70 mg, 0.180 mmol, 1.0 equiv), 8-fluoro-2-methylimidazo[1,2-a]pyridin-6-amine (29.8 mg, 0.180 mmol, 1.0 equiv), DMF (1 mL), DIEA (46.6 mg, 0.360 mmol, 2.0 equiv) and HATU (82.2 mg, 0.217 mmol, 1.2 equiv) was stirred for 3 h at room temperature, then diluted with water (3 mL) and extracted with ethyl acetate (3×4 mL). The organic layers were combined, washed with water (2×10 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 4-[7-({8-fluoro-2-methylimidazo[1,2-a]pyridin-6-yl}carbamoyl)-2-isopropylindazol-4-yl]piperazine-1-carboxylate (60 mg, 62%) as an oil. LCMS (ES, m/z): 536 [M+H]⁺.

### Synthesis of Compound 290

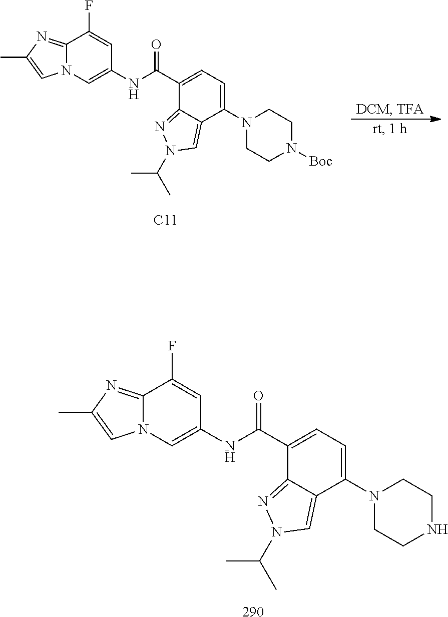

A mixture of tert-butyl 4-[7-({8-fluoro-2-methylimidazo[1,2-a]pyridin-6-yl}carbamoyl)-2-isopropylindazol-4-yl]piperazine-1-carboxylate (60.0 mg, 0.112 mmol, 1.0 equiv), DCM (1 mL) and TFA (1 mL) was stirred for 1 h at room temperature. The resulting mixture was concentrated under reduced pressure to give a residue. The residue was purified by Prep-HPLC (Condition 2, Gradient 1) to afford N-{8-fluoro-2-methylimidazo[1,2-a]pyridin-6-yl}-2-isopropyl-4-(piperazin-1-yl)indazole-7-carboxamide (15 mg, 28%) as a solid. LCMS (ES, m/z): 436 [M+H]⁺. ¹H NMR (300 MHz, DMSO-d₆) δ 11.15 (s, 1H), 9.22 (d, J=1.7 Hz, 1H), 8.82 (s, 1H), 7.99 (d, J=8.1 Hz, 1H), 7.92 (d, J=3.0 Hz, 1H), 7.44-6.97 (m, 1H), 6.49 (d, J=8.2 Hz, 1H), 5.13-4.87 (m, 1H), 3.38-3.34 (m, 4H), 2.94-2.90 (m, 4H), 2.36 (s, 3H), 1.67 (d, J=6.7 Hz, 6H).

### Example 44: Synthesis of Compound 295

### Synthesis of Intermediate C12

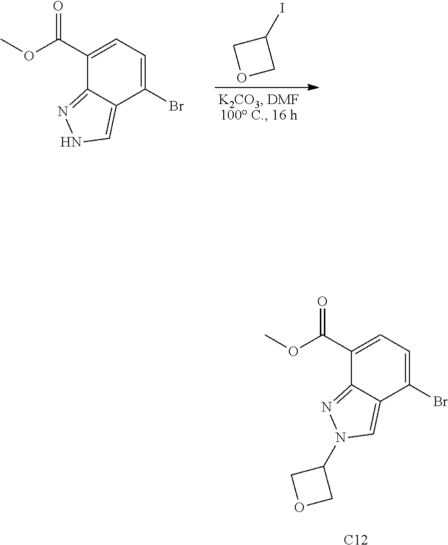

To a stirred mixture of methyl 4-bromo-2H-indazole-7-carboxylate (650 mg, 2.54 mmol, 1.0 equiv) and 3-iodooxetane (937.6 mg, 5.09 mmol, 2 equiv) in DMF (6.5 mL) was added K₂CO₃ (704.3 mg, 5.09 mmol, 2 equiv) at room temperature. The resulting mixture was stirred overnight at 100° C. under nitrogen atmosphere, then cooled to room temperature. The resulting mixture was diluted with water (6 mL) and extracted with ethyl acetate (3×6 mL). The organic layers were combined, washed with brine (lx 10 mL), dried over anhydrous Na₂SO₄, and filtered. After filtration, the filtrate was concentrated under reduced pressure to give a residue. The residue was purified by silica gel column chromatography, eluted with PE/EA (1:1) to afford methyl 4-bromo-2-(oxetan-3-yl)indazole-7-carboxylate (200 mg, 25%) as a solid. LCMS (ES, m/z): 311 [M+H]⁺.

### Synthesis of Intermediate C13

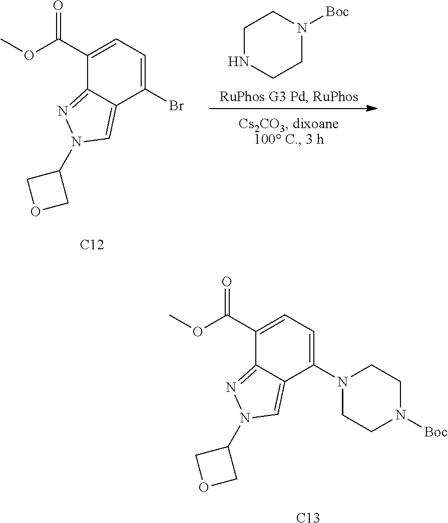

To a stirred mixture of methyl 4-bromo-2-(oxetan-3-yl)indazole-7-carboxylate (200 mg, 0.64 mmol, 1 equiv) and tert-butyl piperazine-1-carboxylate (239.4 mg, 1.28 mmol, 2 equiv) in 1,4-dioxane (2 mL) was added Cs₂CO₃ (628.3 mg, 1.93 mmol, 3 equiv), RuPhos (59.9 mg, 0.13 mmol, 0.2 equiv), and RuPhos G3 Pd (53.7 mg, 0.06 mmol, 0.1 equiv) at room temperature under nitrogen atmosphere. The resulting mixture was stirred for 3 h at 100° C. under nitrogen atmosphere, then cooled to room temperature. The resulting mixture was diluted with water (5 mL) and extracted with ethyl acetate (3×5 mL). The organic layers were combined, washed with brine (lx 5 mL), dried over anhydrous Na₂SO₄, and filtered. After filtration, the filtrate was concentrated under reduced pressure to give a residue. The residue was purified by silica gel column chromatography, eluted with PE/EA (1:1) to afford methyl 4-[4-(tert-butoxycarbonyl)piperazin-1-yl]-2-(oxetan-3-yl)indazole-7-carboxylate (220 mg, 82%) as a solid. LCMS (ES, m/z): 417 [M+H]⁺.

### Synthesis of Intermediate C14

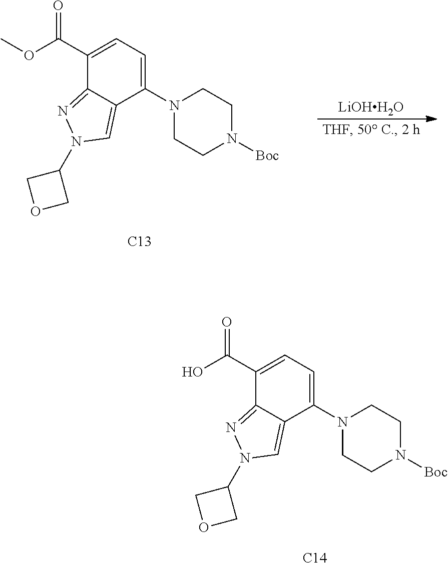

A solution of methyl 4-[4-(tert-butoxycarbonyl)piperazin-1-yl]-2-(oxetan-3-yl)indazole-7-carboxylate (200 mg, 0.4 mmol, 1.0 equiv) in tetrahydrofuran (2 mL) was treated with lithiumol hydrate (80.6 mg, 1.9 mmol, 4.0 equiv) in water (2 mL) at room temperature. The resulting mixture was stirred for 2 h at 50° C. under nitrogen atmosphere, then cooled to 0° C. The resulting mixture was acidified to pH 4 with HCl (1 M) and extracted with ethyl acetate (2×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with EA to afford 4-[4-(tert-butoxycarbonyl)piperazin-1-yl]-2-(oxetan-3-yl)indazole-7-carboxylic acid (160 mg, 83%) as a solid. LCMS (ES, m/z): 403 [M+H]⁺.

### Synthesis of Intermediate C15

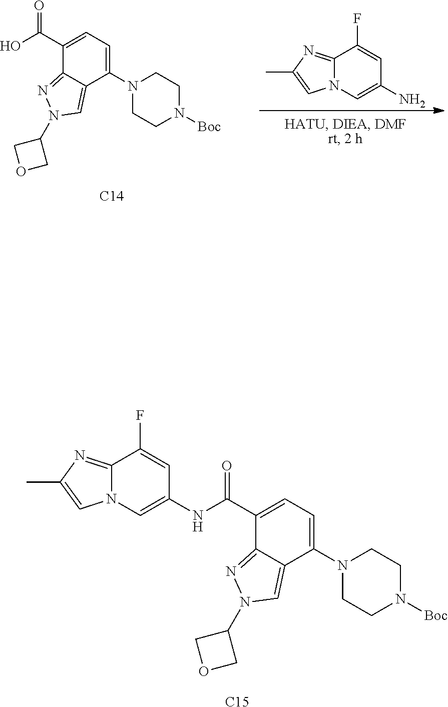

To a stirred mixture of 4-[4-(tert-butoxycarbonyl)piperazin-1-yl]-2-(oxetan-3-yl)indazole-7-carboxylic acid (160 mg, 0.39 mmol, 1.0 equiv) and 8-fluoro-2-methylimidazo[1,2-a]pyridin-6-amine (72.2 mg, 0.43 mmol, 1.1 equiv) in DMF (1 mL) was added DIEA (154.1 mg, 1.19 mmol, 3.0 equiv) and HATU (226.7 mg, 0.59 mmol, 1.5 equiv) at room temperature. The resulting mixture was stirred for 2 h at room temperature under nitrogen atmosphere, then quenched with water and extracted with ethyl acetate (2×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with PE/EA (1:2) to afford tert-butyl 4-[7-({8-fluoro-2-methylimidazo[1,2-a]pyridin-6-yl}carbamoyl)-2-(oxetan-3-yl)indazol-4-yl]piperazine-1-carboxylate (100 mg, 46%) as a solid. LCMS (ES, m/z): 550 [M+H]⁺.

### Synthesis of Compound 295

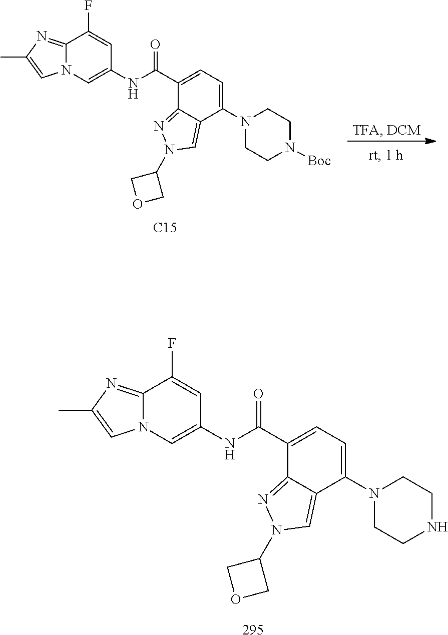

A solution of tert-butyl 4-[7-({8-fluoro-2-methylimidazo[1,2-a]pyridin-6-yl}carbamoyl)-2-(oxetan-3-yl)indazol-4-yl]piperazine-1-carboxylate (100 mg, 0.18 mmol, 1.0 equiv) in DCM (1 mL) was treated with TFA (1 mL) at room temperature. The resulting mixture was stirred for 1 h at room temperature, then concentrated under reduced pressure to give a residue. The residue was purified by Prep-HPLC (Condition 3, Gradient 1) to afford N-{8-fluoro-2-methylimidazo[1,2-a]pyridin-6-yl}-2-(oxetan-3-yl)-4-(piperazin-1-yl)indazole-7-carboxamide trifluoroacetic acid salt (23.3 mg, 28%) as a solid. LCMS (ES, m/z): 450 [M+H]⁺. ¹H NMR (400 MHz, DMSO-d₆) δ 11.17 (s, 1H), 9.38 (s, 1H), 9.16 (s, 1H), 8.85 (s, 2H), 8.27-8.02 (m, 2H), 7.50 (d, J=11.8 Hz, 1H), 6.66 (d, J=8.1 Hz, 1H), 6.12-5.95 (m, 1H), 5.25-5.08 (m, 4H), 3.66-3.58 (m, 4H), 3.37 (s, 4H), 2.41 (s, 3H).

### Example 45: Synthesis of Compound 299

### Synthesis of Intermediate C16

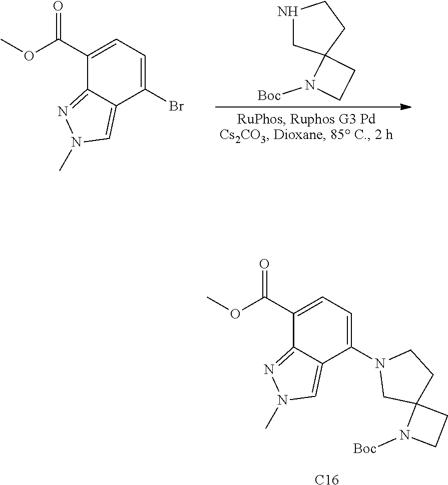

A mixture of methyl 4-bromo-2-methylindazole-7-carboxylate (150.0 mg, 0.037 mmol, 1.0 equiv), tert-butyl 1,6-diazaspiro[3.4]octane-1-carboxylate (153.8 mg, 0.724 mmol, 1.3 equiv), Ruphos (26.0 mg, 0.056 mmol, 0.1 equiv), Dioxane (4 mL), Cs₂CO₃ (363.2 mg, 1.114 mmol, 2.0 equiv) and RuPhos G3 Pd (6.2 mg, 0.007 mmol, 0.2 equiv) was stirred for 2 h at 85° C. under nitrogen atmosphere. The resulting mixture was concentrated under reduced pressure to give a residue. The residue was purified by silica gel column chromatography, eluted with PE/EA (1:1) to afford methyl 4-[1-(tert-butoxycarbonyl)-1,6-diazaspiro[3.4]octan-6-yl]-2-methylindazole-7-carboxylate (170 mg, 70%) as a solid. LCMS (ES, m/z): 401 [M+H]⁺.

### Synthesis of Intermediate C17

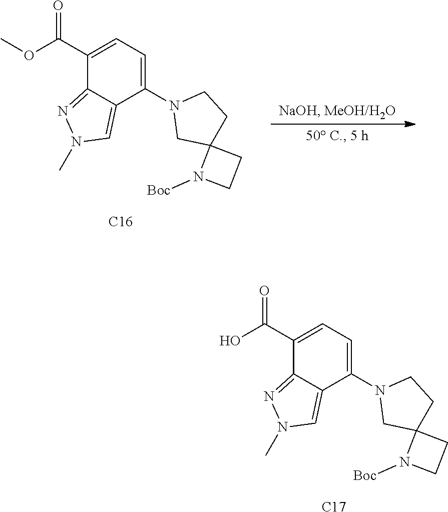

A mixture of methyl 4-[1-(tert-butoxycarbonyl)-1,6-diazaspiro[3.4]octan-6-yl]-2-methylindazole-7-carboxylate (170.0 mg, 0.424 mmol, 1.0 equiv), NaOH (169.7 mg, 4.240 mmol, 10.0 equiv), methanol (3 mL), and water (3 mL) was stirred for 5 h at 50° C. The resulting mixture was dilute d with water (50 mL), acidified to pH 5 with 1 N of HCl, and extracted with ethyl acetate (2×50 mL). The resulting mixture was concentrated under reduced pressure to afford 4-I[1-(tert-butoxycarbonyl)-1,6-diazaspiro[3.4]octan-6-yl]-2-methylindazole-7-carboxylic acid (160 mg, 88%) as a solid. LCMS (ES, m/z): 387 [M+H]⁺.

### Synthesis of Intermediate C18

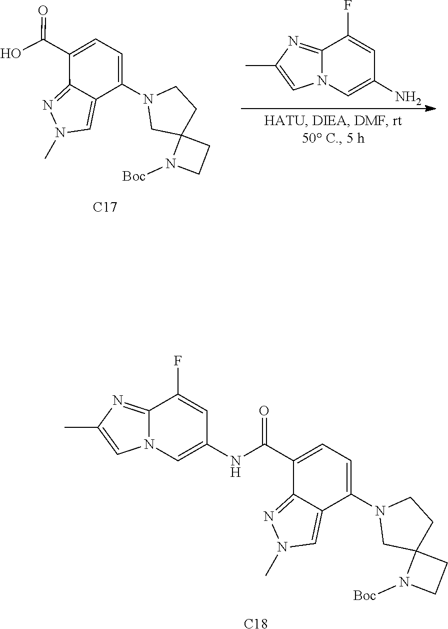

A mixture of 4-[1-(tert-butoxycarbonyl)-1,6-diazaspiro[3.4]octan-6-yl]-2-methylindazole-7-carboxylic acid (50.0 mg, 0.052 mmol, 1.0 equiv), 8-fluoro-2-methylimidazo[1,2-a]pyridin-6-amine (27.7 mg, 0.168 mmol, 1.3 equiv), HATU (98.3 mg, 0.258 mmol, 2.0 equiv), DMF (2.0 mL), and DIEA (50.1 mg, 0.387 mmol, 3.0 equiv) was stirred for 5 h at 50° C. The resulting mixture was diluted with water (20 mL) and extracted with ethyl acetate (2×20 mL). The organic layer were combined, washed with water (1×40 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 6-[7-({8-fluoro-2-methylimidazo[1,2-a]pyridin-6-yl}carbamoyl)-2-methylindazol-4-yl]-1,6-diazaspiro[3.4]octane-1-carboxylate (65 mL, 94%) as a solid. LCMS (ES, m/z): 534 [M+H]⁺.

### Synthesis of Compound 299

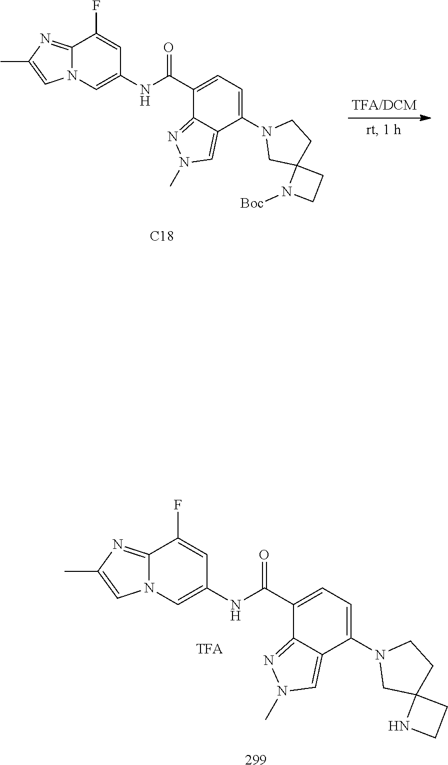

A mixture of tert-butyl 6-[7-({8-fluoroimidazo[1,2-a]pyridin-6-yl}carbamoyl)-2-methylindazol-4-yl]-1,6-diazaspiro[3.4]octane-1-carboxylate (80.0 mg, 0.154 mmol, 1.0 equiv), DCM (1 mL), an d TFA (1 mL) was stirred for 1 h at room temperature. The resulting mixture was concentrated under reduced pressure to give a residue. The residue was purified by Prep-HPLC (Condition 3, Gradient 2) to afford 4-{1,6-diazaspiro[3.4]octan-6-yl}-N-{8-fluoro-2-methylimidazo[1,2-a]pyridin-6-yl}-2-methylindazole-7-carboxamide (45.3 mg, 66%) as a solid. LCMS (ES, m/z): 434 [M+H]⁺. ¹H NMR (300 MHz, DMSO-d₆) δ 11.18 (s, 1H), 9.41 (d, J=1.6 Hz, 1H), 9.10-9.09 (m, 1H), 9.00-8.97 (m, 1H), 8.90 (s, 1H), 8.09 (s, 1H), 7.99 (d, J=8.2 Hz, 1H), 7.73 (d, J=12.0 Hz, 1H), 6.10 (d, J=8.4 Hz, 1H), 4.44 (d, J=12.4 Hz, 1H), 4.31 (s, 3H), 4.00-3.53 (m, 5H), 2.81 (dd, J=13.6, 5.8 Hz, 1H), 2.66 (t, J=8.1 Hz, 2H), 2.43 (s, 4H).

### Example 46: Synthesis of Compound 316

### Synthesis of Intermediate C19

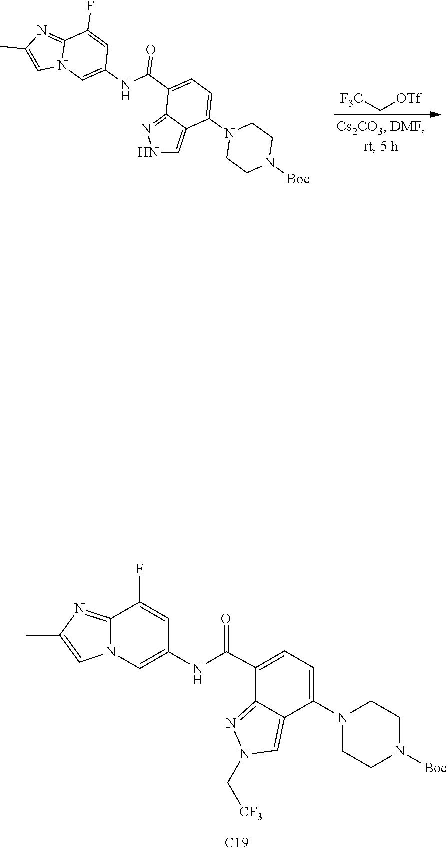 [SMILES: Cc1cn2cc(NC(=O)c3ccc(N4CCN(C(=O)OC(C)(C)C)CC4)c4c[nH]nc34)cc(F)c2n1 ; O=S(=O)(OCC(F)(F)F)C(F)(F)F ; Cc1cn2cc(NC(=O)c3ccc(N4CCN(C(=O)OC(C)(C)C)CC4)c4cn(CC(F)(F)F)nc34)cc(F)c2n1 ; "C19"]

To a stirred mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.20 mmol, 1.0 equiv) and 2,2,2-trifluoroethyl trifluoromethanesulfonate (70.5 mg, 0.30 mmol, 1.5 equiv) in DMF (1 mL) was added Cs₂CO₃ (198.0 mg, 0.60 mmol, 3.0 equiv) at room temperature. The resulting mixture was stirred for 5 h at room temperature, then quenched with water (2 mL) and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[7-({8-fluoro-2-methylimidazo[1,2-a]pyridin-6-yl}carbamoyl)-2-(2,2,2-trifluoroethyl)indazol-4-yl]piperazine-1-carboxylate (52 mg, 44.59%) as a solid. LCMS (ES, m/z): 576 [M+H]⁺.

### Synthesis of Compound 316

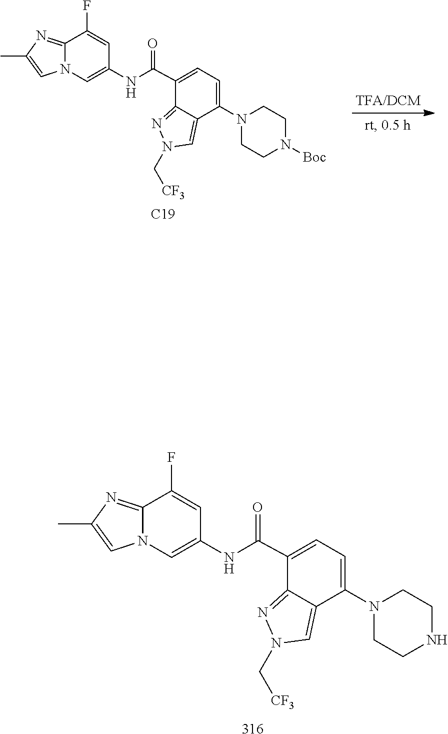 [SMILES: Cc1cn2cc(NC(=O)c3ccc(N4CCN(C(=O)OC(C)(C)C)CC4)c4cn(CC(F)(F)F)nc34)cc(F)c2n1 ; "C19" ; Cc1cn2cc(NC(=O)c3ccc(N4CCNCC4)c4cn(CC(F)(F)F)nc34)cc(F)c2n1 ; "316"]

A solution of tert-butyl 4-[7-({8-fluoro-2-methylimidazo[1,2-a]pyridin-6-yl}carbamoyl)-2-(2,2,2-trifluoroethyl)indazol-4-yl]piperazine-1-carboxylate (47.0 mg, 0.08 mmol, 1 equiv) in DCM (0.5 mL) was treated with TFA (0.5 mL) at room temperature. The resulting mixture was stirred for 30 min at room temperature, then concentrated under reduced pressure to give a residue. The residue was purified by Prep-HPLC (Condition 3, Gradient 3) to afford N-{8-fluoro-2-methylimidazo[1,2-a]pyridin-6-yl}-4-(piperazin-1-yl)-2-(2,2,2-trifluoroethyl)indazole-7-carboxamide trifluoroacetic acid salt (6 mg, 15%) as a solid. LCMS (ES, m/z): 476 [M+H]⁺. ¹H NMR (300 MHz, DMSO-d6) δ 11.15 (s, 1H), 9.43 (s, 1H), 9.31 (s, 2H), 9.14 (s, 1H), 8.18-8.00 (m, 2H), 7.64 (d, J=11.8 Hz, 1H), 6.65 (d, J=8.1 Hz, 1H), 5.69 (q, J=9.0 Hz, 2H), 3.67 (s, 4H), 3.36 (s, 4H), 2.42 (s, 3H).

### Example 47: Synthesis of Compound 304

### Synthesis of Intermediate C20

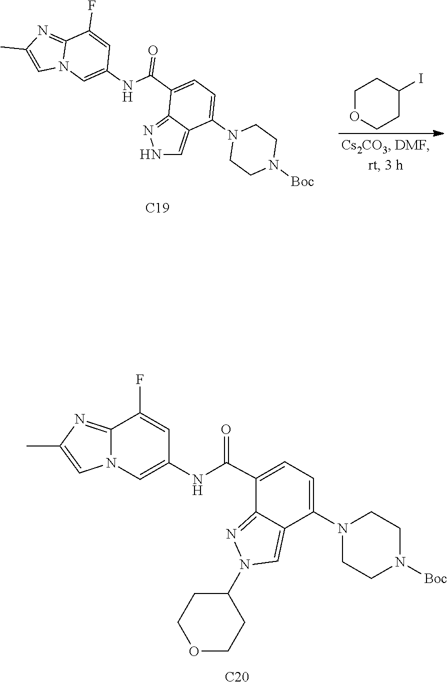 [SMILES: Cc1cn2cc(NC(=O)c3ccc(N4CCN(C(=O)OC(C)(C)C)CC4)c4c[nH]nc34)cc(F)c2n1 ; IC1CCOCC1 ; "C19" ; Cc1cn2cc(NC(=O)c3ccc(N4CCN(C(=O)OC(C)(C)C)CC4)c4cn(C5CCOCC5)nc34)cc(F)c2n1 ; "C20"]

To a stirred mixture of tert-butyl 4-[7-({8-fluoro-2-methylimidazo[1,2-a]pyridin-6-yl}carbamoyl)-2H-indazol-4-yl]piperazine-1-carboxylate (100 mg, 0.20 mmol, 1.0 equiv) and 4-iodooxane (64.4 mg, 0.30 mmol, 1.5 equiv) in DMF (1 mL) was added Cs₂CO₃ (198.0 mg, 0.61 mmol, 3.0 equiv) at room temperature. The resulting mixture was stirred for 3 h at room temperature, then quenched with water at room temperature and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with EA to afford tert-butyl 4-[7-({8-fluoro-2-methylimidazo[1,2-a]pyridin-6-yl}carbamoyl)-2-(oxan-4-yl)indazol-4-yl]piperazine-1-carboxylate (41 mg, 35%) as a solid. LCMS (ES, m/z): 578 [M+H]⁺.

### Synthesis of Compound 394

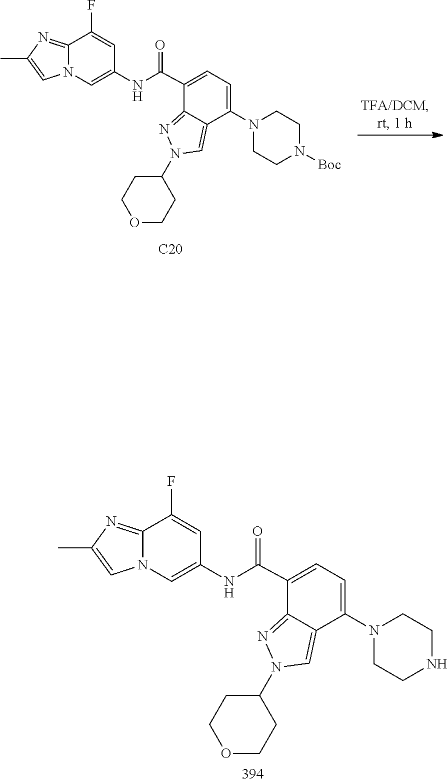

To a stirred mixture of tert-butyl 4-[7-({8-fluoro-2-methylimidazo[1,2-a]pyridin-6-yl}carbamoyl)-2-(oxan-4-yl)indazol-4-yl]piperazine-1-carboxylate (40 mg, 0.07 mmol, 1.0 equiv) in DCM (0.4 mL) was added TFA (0.4 mL) at room temperature. The resulting mixture was stirred for 1 h at room temperature, then concentrated under reduced pressure to give a residue. The residue was purified by Prep-HPLC (Condition 3, Gradient 4) to afford N-{8-fluoro-2-methylimidazo[1,2-a]pyridin-6-yl}-2-(oxan-4-yl)-4-(piperazin-1-yl)indazole-7-carboxamide trifluoroacetic acid salt (22.8 mg, 50%) as a solid. LCMS (ES, m/z): 478 [M+H]⁺. ¹H NMR (300 MHz, DMSO-d₆) δ 11.17 (s, 1H), 9.44 (d, J=1.6 Hz, 1H), 9.15 (s, 2H), 9.00 (s, 1H), 8.13 (d, J=2.6 Hz, 1H), 8.04 (d, J=8.0 Hz, 1H), 7.71 (dd, J=11.8, 1.6 Hz, 1H), 6.62 (d, J=8.1 Hz, 1H), 4.95 (tt, J=10.4, 5.5 Hz, 1H), 4.10 (dt, J=11.2, 3.2 Hz, 2H), 3.69-3.51 (m, 6H), 3.37-3.35 (m, 4H), 2.44 (s, 3H), 2.25 (td, J=10.3, 9.5, 4.1 Hz, 4H).

### Example 48: Synthesis of Compound 357

### Synthesis of Intermediate C22

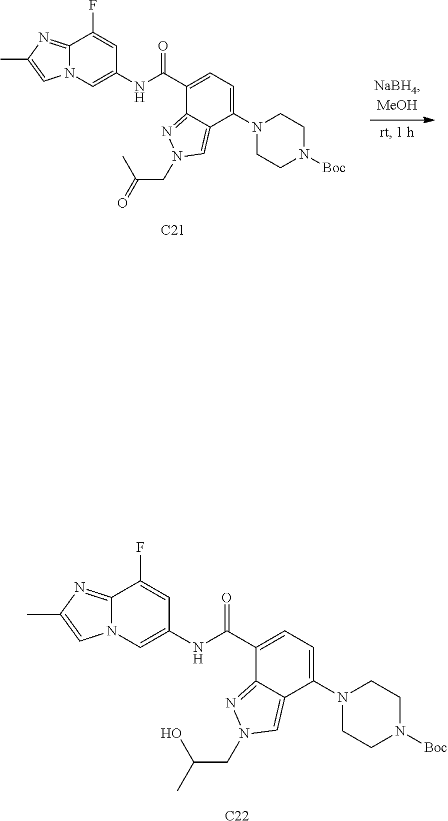

A solution of tert-butyl 4-[7-({8-fluoro-2-methylimidazo[1,2-a]pyridin-6-yl}carbamoyl)-2-(2-oxopropyl)indazol-4-yl]piperazine-1-carboxylate (90.0 mg, 0.16 mmol, 1.0 equiv) in methanol (1 mL) was treated with NaBH₄ (7.4 mg, 0.19 mmol, 1.2 equiv) at 0° C. under nitrogen atmosphere. The resulting mixture was stirred for 1 h at room temperature, then quenched with sat. NH₄Cl (aq.) at 0° C. and extracted with ethyl acetate (2×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with PE/EA (1:4) to afford tert-butyl 4-[7-({8-fluoro-2-methylimidazo[1,2-a]pyridin-6-yl}carbamoyl)-2-(2-oxopropyl)indazol-4-yl]piperazine-1-carboxylate (57 mg, 63%) as a solid. LCMS (ES, m/z): 552 [M+H]⁺.

### Synthesis of Compound 357

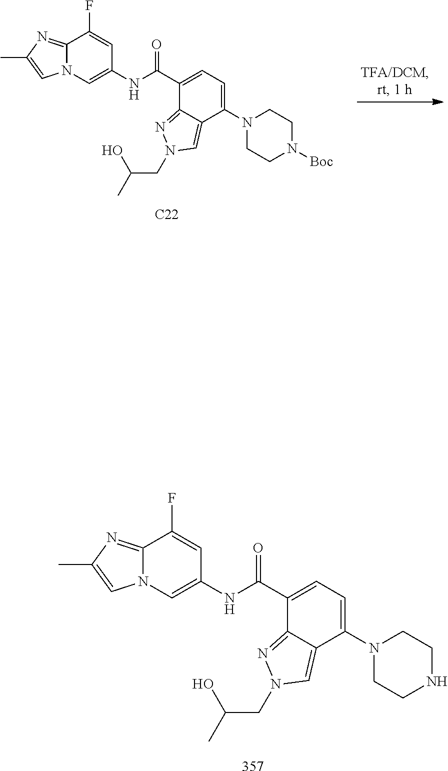

A solution of tert-butyl 4-[7-({8-fluoro-2-methylimidazo[1,2-a]pyridin-6-yl}carbamoyl)-2-(2-oxopropyl)indazol-4-yl]piperazine-1-carboxylate (57.0 mg, 0.10 mmol, 1.0 equiv) in DCM (0.6 mL) was treated with TFA (0.6 mL) at room temperature. The resulting mixture was stirred for 1 h at room temperature, then concentrated under reduced pressure to give a residue. The residue was purified by Prep-HPLC (Condition 3, Gradient 5) to afford N-{8-fluoro-2-methylimidazo[1,2-a]pyridin-6-yl}-2-(2-oxopropyl)-4-(piperazin-1-yl)indazole-7-carboxamide trifluoroacetic acid salt (1.7 mg, 3%) as a solid. LCMS (ES, m/z): 452 [M+H]⁺. ¹H NMR (400 MHz, Methanol-d₄) δ 9.54 (s, 1H), 8.66 (s, 1H), 8.19 (d, J=7.9 Hz, 1H), 8.09 (s, 1H), 8.02 (d, J=11.3 Hz, 1H), 6.69 (d, J=8.0 Hz, 1H), 4.73-4.60 (m, 1H), 4.48 (dd, J=23.1, 10.0 Hz, 2H), 3.71 (t, J=5.0 Hz, 4H), 3.52 (t, J=5.1 Hz, 4H), 2.59 (s, 3H), 1.35 (d, J=6.1 Hz, 3H).

### Example 49: Synthesis of Compound 354

### Synthesis of Intermediate C23

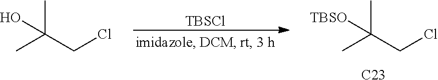

A mixture of 1-chloro-2-methyl-2-propanol (1.5 g, 13.816 mmol, 1.0 equiv), DCM (15 mL), imidazole (1.8 g, 27.615 mmol, 2.0 equiv), and t-butyldimethylchlorosilane (3.1 g, 20.701 mmol, 1.5 equiv) was stirred for 3 h at room temperature. The reaction mixture was quenched with water (100 mL) at room temperature and extracted with CH₂Cl₂ (2×100 mL). The organic layers were combined, dried over Na₂SO₄, and filtered. The filtrate was concentrated under reduced pressure to give a residue. The residue was purified by silica gel column chromatography, eluted with PE/EA (5:1) to afford tert-butyl[(1-chloro-2-methylpropan-2-yl)oxy]dimethylsilane (1.2 g, 35%) as an oil.

### Synthesis of Intermediate C24

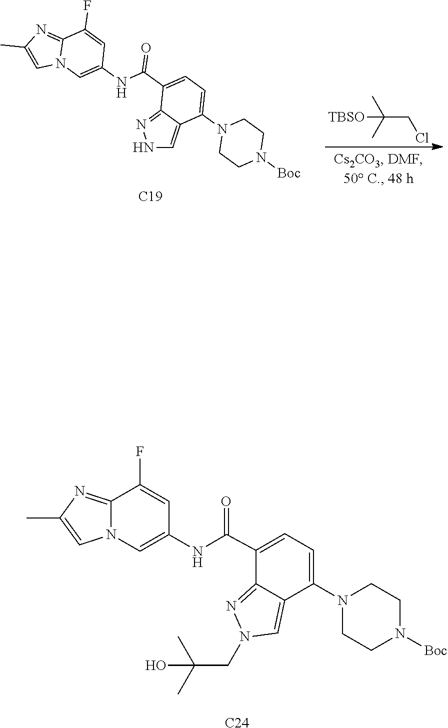 [SMILES: Cc1cn2cc(NC(=O)c3ccc(N4CCN(C(=O)OC(C)(C)C)CC4)c4c[nH]nc34)cc(F)c2n1 ; CC(C)(CCl)O[Si](C)(C)C(C)(C)C ; "C19" ; Cc1cn2cc(NC(=O)c3ccc(N4CCN(C(=O)OC(C)(C)C)CC4)c4cn(CC(C)(C)O)nc34)cc(F)c2n1 ; "C24"]

A mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.203 mmol, 1.0 equiv), Cs₂CO₃ (198.66 mg, 0.609 mmol, 3 equiv), dimethylformamide (2 mL), and tert-butyl[(1-chloro-2-methylpropan-2-yl)oxy]dimethylsilane (135.4 mg, 0.609 mmol, 3.0 equiv) was stirred for 48 h at 50° C. The reaction was quenched with water (5 mL) at room temperature and extracted with ethyl acetate (2×5 mL). The organic layers were combined, dried over Na₂SO₄, and filtered. The filtrate was concentrated under reduced pressure to give a residue. The residue was purified by silica gel column chromatography, eluted with CH₂Cl₂/MeOH (30:1) to afford tert-butyl 4-[7-({8-fluoro-2-methylimidazo[1,2-a]pyridin-6-yl}carbamoyl)-2-(2-hydroxy-2-methylpropyl) indazol-4-yl]piperazine-1-carboxylate (100 mg, 79%) as a solid. LCMS (ES, m/z): 566 [M+H]⁺.

### Synthesis of Compound 354

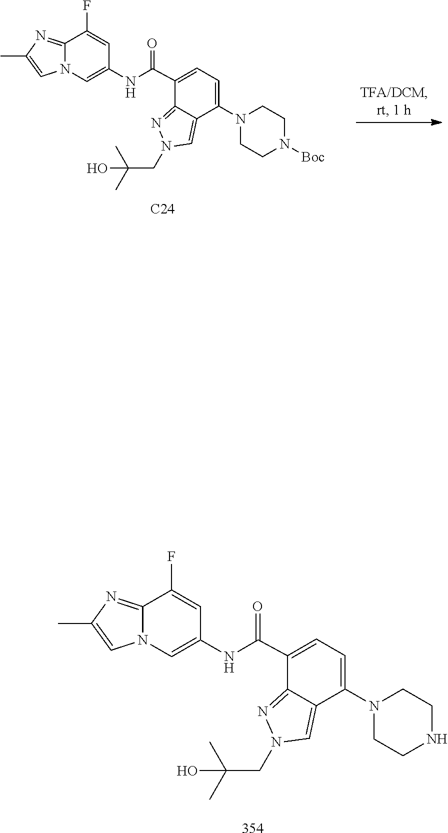 [SMILES: Cc1cn2cc(NC(=O)c3ccc(N4CCN(C(=O)OC(C)(C)C)CC4)c4cn(CC(C)(C)O)nc34)cc(F)c2n1 ; "C24" ; Cc1cn2cc(NC(=O)c3ccc(N4CCNCC4)c4cn(CC(C)(C)O)nc34)cc(F)c2n1 ; "354"]

A mixture of tert-butyl 4-[7-({8-fluoro-2-methylimidazo[1,2-a]pyridin-6-yl}carbamoyl)-2-(2-hydroxy-2-methylpropyl)indazol-4-yl]piperazine-1-carboxylate (60.0 mg, 0.106 mmol, 1.0 equiv), DCM (1 mL), and trifluoroacetaldehyde (1 mL) was stirred for 1 h at room temperature. The resulting mixture was concentrated under reduced pressure to give a residue. The residue was purified by Prep-HPLC (Condition 3, Gradient 2) to afford N-{8-fluoro-2-methylimidazo[1,2-a]pyridin-6-yl}-2-(2-hydroxy-2-methylpropyl)-4-(piperazin-1-yl)indazole-7-carboxamide trifluoroacetic acid salt (37.1 mg, 75%) as a solid. LCMS (ES, m/z): 466 [M+H]⁺. ¹H NMR (300 MHz, DMSO-d₆) δ 11.41 (s, 1H), 9.46 (d, J=1.6 Hz, 1H), 9.03 (s, 2H), 8.82 (s, 1H), 8.16-8.09 (m, 1H), 8.04 (d, J=8.0 Hz, 1H), 7.60 (d, J=11.9 Hz, 1H), 6.63 (d, J=8.1 Hz, 1H), 4.51 (s, 2H), 3.66-3.57 (m, 4H), 3.37-3.36 (m, 4H), 2.43 (s, 3H), 1.23 (s, 6H).

### Example 50: Synthesis of Compound 358

### Synthesis of Intermediate C25

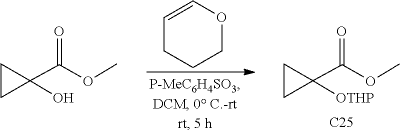

Methyl 1-hydroxycyclopropane-1-carboxylate (2.0 g, 17.224 mmol, 1.0 equiv), DCM (30 mL), PPTS (1.3 g, 5.167 mmol, 0.3 equiv), and dihydropyran (2.1 g, 25.836 mmol, 1.5 equiv) were combined at 0° C. The resulting mixture was stirred for 5 h at room temperature, then quenched with water (100 mL) at room temperature and extracted with CH₂Cl₂ (2×100 mL). The organic layers were combined, dried by Na₂SO₄, and filtered. The filtrate was concentrated under reduced pressure to give a residue. The residue was purified by silica gel column chromatography, eluted with PE/EA (5:1) to afford methyl 1-(oxan-2-yloxy)cyclopropane-1-carboxylate (2.5 g, 65%) as a solid. ¹H NMR (300 MHz, DMSO-d₆) δ 4.83-4.81 (m, 1H), 3.79-3.71 (m, 1H), 3.64 (s, 3H), 3.52-3.36 (m, 1H), 1.84-1.62 (m, 2H), 1.61-1.32 (m, 5H), 1.27-1.08 (m, 3H).

### Synthesis of Intermediate C26

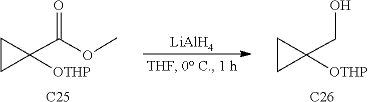

Methyl 1-(oxan-2-yloxy)cyclopropane-1-carboxylate (400.0 mg, 1.998 mmol, 1.0 equiv), tetrahydrofuran (10 mL), and LiAlH₄ (113.7 mg, 2.997 mmol, 1.5 equiv) were combined at 0° C. The resulting mixture was stirred for 1 h at room temperature under nitrogen atmosphere, quenched with water (100 mL) at 0° C. and extracted with ethyl acetate (2×100 mL). The organic layers were combined, dried over Na₂SO₄, and filtered. The resulting mixture was concentrated under reduced pressure to give a solid.

### Synthesis of Intermediate C27

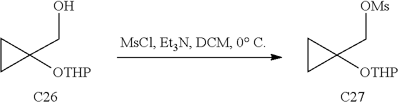

[1-(oxan-2-yloxy)cyclopropyl]methanol (300 mg, 1.742 mmol, 1.0 equiv), DCM (6 mL), triethylamine (352.5 mg, 3.484 mmol, 2.0 equiv), and methanesulfonyl chloride (399.0 mg, 3.484 mmol, 2.0 equiv) were combined at 0° C. The resulting mixture was stirred for 2 h at 0° C., then quenched with water (50 mL) at 0° C. and extracted with CH₂Cl₂ (2×50 mL). The organic layers were combined, dried over Na₂SO₄, and filtered. The filtrate was concentrated under reduced pressure to give a solid.

### Synthesis of Intermediate C28

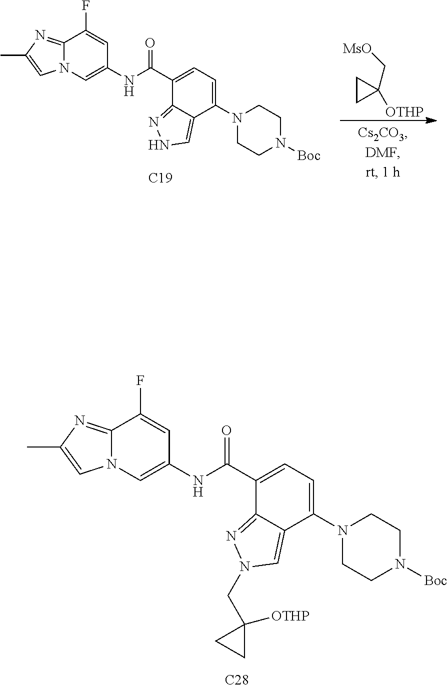

A mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.203 mmol, 1.0 equiv), Cs₂CO₃ (198.6 mg, 0.609 mmol, 3.0 equiv), dimethylformamide (2 mL), and [1-(oxan-2-yloxy)cyclopropyl]methyl methanesulfonate (152.1 mg, 0.609 mmol, 3.0 equiv) was stirred overnight at 50° C. The reaction mixture was quenched with water (10 mL) at room temperature and extracted with ethyl acetate (2×10 mL). The organic layer were combined, dried over Na₂SO₄, and filtered. The filtrate was concentrated under reduced pressure to give a residue. The residue was purified by silica gel column chromatography, eluted with CH₂Cl₂/MeOH (30:1) to afford tert-butyl 4-[7-({8-fluoro-2-methylimidazo[1,2-a]pyridin-6-yl}carbamoyl)-2-{[1-(oxan-2-yloxy)cyclopropyl]methyl}indazol-4-yl]piperazine-1-carboxylate (100 mg, 70%) as a solid. LCMS (ES, m/z): 648 [M+H]⁺.

### Synthesis of Compound 358

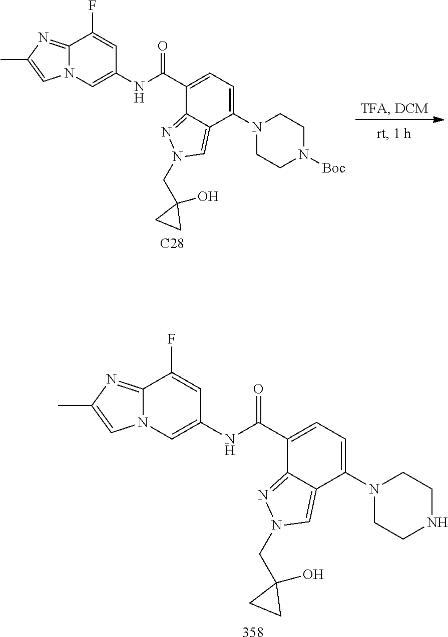

A mixture of tert-butyl 4-[7-({8-fluoro-2-methylimidazo[1,2-a]pyridin-6-yl}carbamoyl)-2-[(1-hydroxycyclopropyl)methyl]indazol-4-yl]piperazine-1-carboxylate (100.0 mg, 0.177 mmol, 1.0 equiv), DCM (1 mL), and trifluoroacetaldehyde (1 mL) was stirred for 1 h at room temperature. The resulting mixture was concentrated under reduced pressure to give a residue. The residue was purified by Prep-HPLC (Condition 3, Gradient 2) to afford N-{8-fluoro-2-methylimidazo[1,2-a]pyridin-6-yl}-2-[(1-hydroxycyclopropyl)methyl]-4-(piperazin-1-yl)indazole-7-carboxamide trifluoroacetic acid salt (29.4 mg, 35%) as a solid. LCMS (ES, m/z): 464 [M+H]⁺. ¹H NMR (300 MHz, DMSO-d₆) δ 11.42 (s, 1H), 9.46 (d, J=1.6 Hz, 1H), 9.01 (s, 2H), 8.88 (s, 1H), 8.12 (d, J=2.7 Hz, 1H), 8.05 (d, J=8.0 Hz, 1H), 7.61 (d, J=11.7 Hz, 1H), 6.64 (d, J=8.1 Hz, 1H), 4.63 (s, 2H), 3.62 (t, J=5.1 Hz, 4H), 3.373.36 (m, 4H), 2.42 (s, 3H), 1.00-0.83 (m, 2H), 0.83-0.74 (m, 2H).

### Example 51: Synthesis of Compound 305

### Synthesis of Intermediate C29

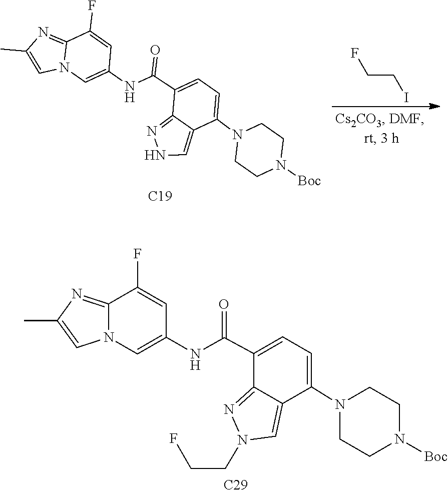

To a stirred mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.20 mmol, 1 equiv) and 1-fluoro-2-iodo-ethane, (52.8 mg, 0.30 mmol, 1.5 equiv) in DMF (1 mL) was added Cs₂CO₃ (198.0 mg, 0.60 mmol, 3.0 equiv) at room temperature. The resulting mixture was stirred for 3 h at room temperature, then diluted with water and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[7-({8-fluoro-2-methylimidazo[1,2-a]pyridin-6-yl}carbamoyl)-2-(2-fluoroethyl)indazol-4-yl]piperazine-1-carboxylate (41 mg, 38%) as a solid. LCMS (ES, m/z): 540 [M+H]⁺.

### Synthesis of Compound 305

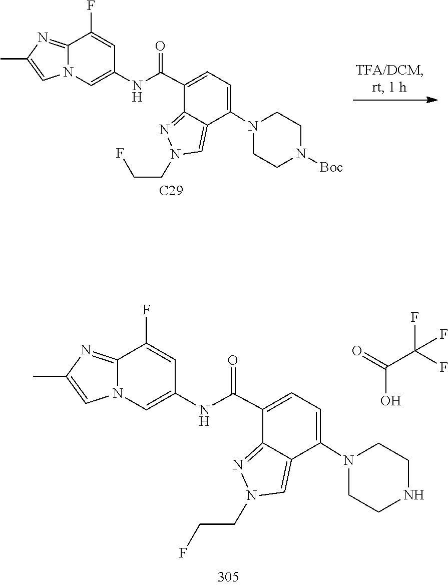

To a stirred mixture of tert-butyl 4-[7-({8-fluoro-2-methylimidazo[1,2-a]pyridin-6-yl}carbamoyl)-2-(2-fluoroethyl)indazol-4-yl]piperazine-1-carboxylate (41.0 mg, 0.07 mmol, 1 equiv) in DCM (0.4 mL) was added TFA (0.4 mL) at room temperature. The resulting mixture was stirred for 1 h at room temperature, then concentrated under reduced pressure to give a residue. The residue was purified by Prep-HPLC (Condition 3, Gradient 6) to afford N-{8-fluoro-2-methylimidazo[1,2-a]pyridin-6-yl}-2-(2-fluoroethyl)-4-(piperazin-1-yl)indazole-7-carboxamide trifluoroacetic acid (24.3 mg, 49%) as a solid. LCMS (ES, m/z): 440 [M+H]⁺. ¹H NMR (300 MHz, DMSO-d₆) δ 11.19 (s, 1H), 9.43 (d, J=1.5 Hz, 1H), 9.01 (s, 1H), 8.95 (s, 2H), 8.10 (s, 1H), 8.06 (d, J=8.0 Hz, 1H), 7.70 (d, J=12.0 Hz, 1H), 6.64 (d, J=8.1 Hz, 1H), 5.16 (t, J=4.6 Hz, 1H), 5.01 (t, J=4.6 Hz, 1H), 4.96 (t, J=4.7 Hz, 1H), 4.84 (t, J=4.7 Hz, 1H), 3.61 (t, J=5.2 Hz, 4H), 3.37 (s, 4H), 2.42 (d, J=0.9 Hz, 3H).

### Example 52: Synthesis of Compound 302

### Synthesis of Intermediate C30

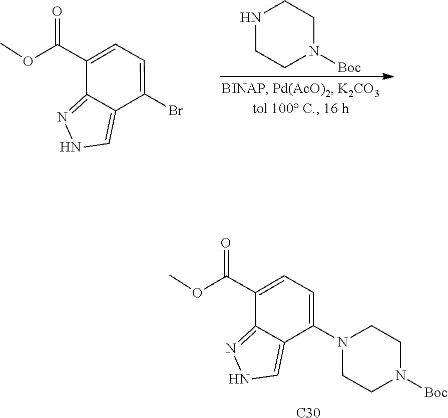

To a stirred mixture of methyl 4-bromo-2H-indazole-7-carboxylate (10 g, 39.2 mmol, 1.0 equiv) and tert-butyl piperazine-1-carboxylate (10.9 g, 58.8 mmol, 1.5 equiv) in toluene (100 mL) was added K₂CO₃ (16.2 g, 117.6 mmol, 3 equiv), BINAP (4.8 g, 7.8 mmol, 0.2 equiv), and Pd(AcO)₂ (0.8 g, 3.9 mmol, 0.1 equiv) at room temperature. The resulting mixture was stirred overnight at 100° C. under nitrogen atmosphere, then cooled to room temperature. The resulting mixture was diluted with water (100 mL) and extracted with ethyl acetate (2×100 mL). The organic layers were combined, washed with brine (1×100 mL), dried over anhydrous Na₂SO₄, and filtered. After filtration, the filtrate was concentrated under reduced pressure to give a residue. The residue was purified by silica gel column chromatography, eluted with PE/EA (1:1) to afford methyl 4-[4-(tert-butoxycarbonyl)piperazin-1-yl]-2H-indazole-7-carboxylate (10.4 g, 74%) as a solid. LCMS (ES, m/z): 361 [M+H]⁺.

### Synthesis of Intermediate C31

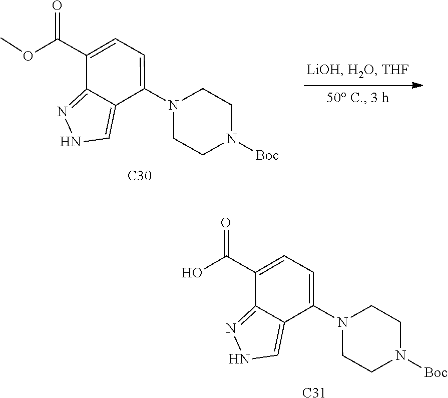

A solution of methyl 4-[4-(tert-butoxycarbonyl)piperazin-1-yl]-211-indazole-7-carboxylate (10.4 g, 28.8 mmol, 1 equiv) in THE (100 mL) was treated with LiOH·H₂O (2.7 g, 115.4 mmol, 4 equiv) in water (100 mL) at room temperature. The resulting mixture was stirred for 2 h at 50° C. under nitrogen atmosphere, then cooled to 0° C. The resulting mixture was acidified to pH 4 with HCl (1 M) and extracted with ethyl acetate (3×150 mL). The organic layers were combined, washed with brine (1×100 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (15:1) to afford 4-[4-(tert-butoxycarbonyl)piperazin-1-yl]-2H-indazole-7-carboxylic acid (9.6 g, 96%) as a solid. LCMS (ES, m/z): 347 [M+H]f.

### Synthesis of Intermediate C32

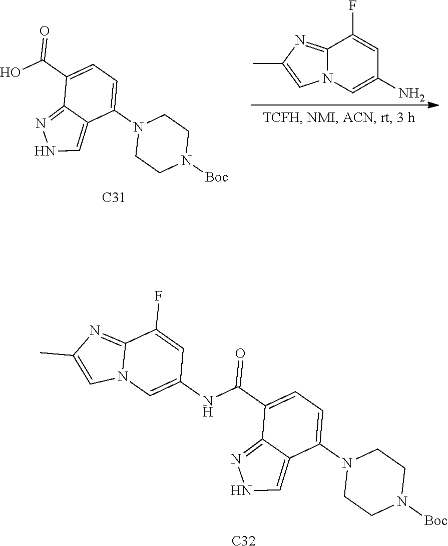 [SMILES: CC(C)(C)OC(=O)N1CCN(c2ccc(C(=O)O)c3n[nH]cc23)CC1 ; Cc1cn2cc(N)cc(F)c2n1 ; "C31" ; Cc1cn2cc(NC(=O)c3ccc(N4CCN(C(=O)OC(C)(C)C)CC4)c4c[nH]nc34)cc(F)c2n1 ; "C32"]

To a stirred mixture of 4-[4-(tert-butoxycarbonyl)piperazin-1-yl]-2H-indazole-7-carboxylic acid (9.6 g, 27.7 mmol, 1.0 equiv), 8-fluoro-2-methylimidazo[1,2-a]pyridin-6-amine (5.0 g, 30.4 mmol, 1.1 equiv), and NMI (9.1 g, 110.8 mmol, 4 equiv) in acetonitrile (100 mL) was added TCFH (9.3 g, 33.2 mmol, 1.2 equiv) at room temperature. The resulting mixture was stirred for 2 h at room temperature under nitrogen atmosphere, then diluted with water and extracted with ethyl acetate (3×100 mL). The organic layers were combined, washed with brine (1×100 mL), dried over anhydrous Na₂SO₄, and filtered. After filtration, the filtrate was concentrated under reduced pressure to give a residue. The residue was purified by silica gel column chromatography, eluted with PE/EA (1:4) to afford tert-butyl 4-[7-({8-fluoro-2-methylimidazo[1,2-a]pyridin-6-yl}carbamoyl)-2H-indazol-4-yl]piperazine-1-carboxylate (10.6 g, 77%) as a solid. LCMS (ES, m/z): 494 [M+H]f.

### Synthesis of Intermediate C33

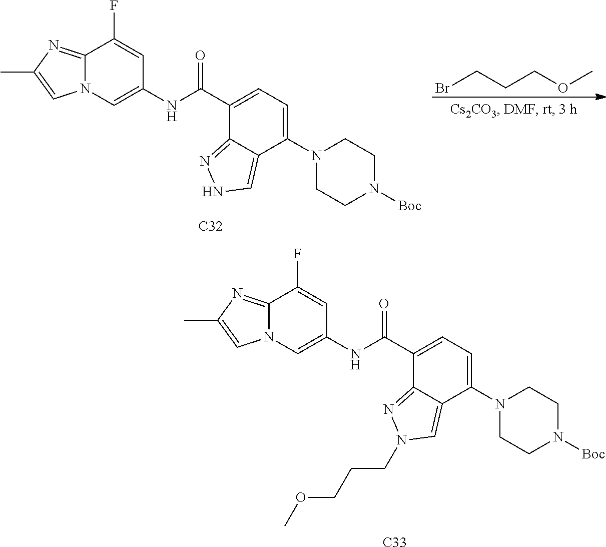

To a stirred mixture of tert-butyl 4-[7-({8-fluoro-2-methylimidazo[1,2-a]pyridin-6-yl}carbamoyl)-2H-indazol-4-yl]piperazine-1-carboxylate (60 mg, 0.12 mmol, 1 equiv) and 1-bromo-3-methoxypropane (27.9 mg, 0.18 mmol, 1.5 equiv) in DMF (0.6 mL) was added Cs₂CO₃ (118.8 mg, 0.37 mmol, 3 equiv) at room temperature. The resulting mixture was stirred for 3 h at room temperature, then diluted with water and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[7-({8-fluoro-2-methylimidazo[1,2-a]pyridin-6-yl}carbamoyl)-2-(3-methoxypropyl)indazol-4-yl]piperazine-1-carboxylate (34 mg, 49%) as a solid. LCMS (ES, m/z): 566 [M+H]⁺.

### Synthesis of Compound 302

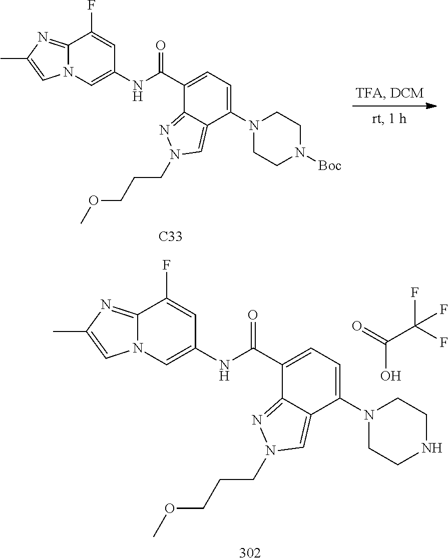

To a stirred mixture of tert-butyl 4-[7-({8-fluoro-2-methylimidazo[1,2-a]pyridin-6-yl}carbamoyl)-2-(3-methoxypropyl)indazol-4-yl]piperazine-1-carboxylate (34 mg, 0.06 mmol, 1 equiv) in DCM (0.4 mL) was added TFA (0.4 mL) at room temperature. The resulting mixture was stirred for 1 h at room temperature, then concentrated under reduced pressure to give a residue. The residue was purified by Prep-HPLC (Condition 3, Gradient 7) to afford N-{8-fluoro-2-methylimidazo[1,2-a]pyridin-6-yl}-2-(3-methoxypropyl)-4-(piperazin-1-yl)indazole-7-carboxamide trifluoroacetic acid salt (14.1 mg, 50%) as a solid. LCMS (ES, m/z): 466 [M+H]⁺. ¹H NMR (300 MHz, DMSO-d₆) δ 11.19 (s, 1H), 9.39 (d, J=1.6 Hz, 1H), 8.96-8.93 (m, 3H), 8.09 (d, J=2.1 Hz, 1H), 8.04 (d, J=8.0 Hz, 1H), 7.62 (d, J=12.0 Hz, 1H), 6.62 (d, J=8.1 Hz, 1H), 4.63 (t, J=7.1 Hz, 2H), 3.59-3.57 (m, 4H), 3.42 (t, J=6.1 Hz, 2H), 3.36-3.34 (m, 4H), 3.28 (s, 3H), 2.42 (s, 3H), 2.30 (q, J=6.5 Hz, 2H).

### Example 53: Synthesis of Compound 338

### Synthesis of Intermediate C34

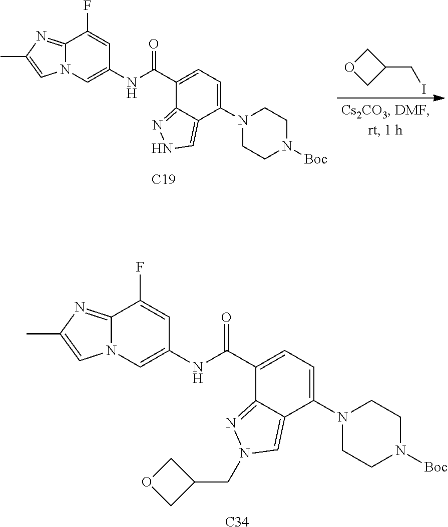 [SMILES: Cc1cn2cc(NC(=O)c3ccc(N4CCN(C(=O)OC(C)(C)C)CC4)c4c[nH]nc34)cc(F)c2n1 ; ICC1COC1 ; "C19" ; Cc1cn2cc(NC(=O)c3ccc(N4CCN(C(=O)OC(C)(C)C)CC4)c4cn(CC5COC5)nc34)cc(F)c2n1 ; "C34"]

To a stirred solution of tert-butyl 4-[7-({8-fluoro-2-methylimidazo[1,2-a]pyridin-6-yl}carbamoyl)-2H-indazol-4-yl]piperazine-1-carboxylate (110 mg, 0.223 mmol, 1.0 equiv) and 3-(iodomethyl)oxetane (66.20 mg, 0.335 mmol, 1.5 equiv) in DMF (2.2 mL) was added Cs₂CO₃ (217.8 mg, 0.669 mmol, 3.0 equiv) at room temperature. The resulting mixture was stirred for 1 h at room temperature, then diluted with water (10 mL) and extracted with ethyl acetate (3×10 mL). The organic layer were combined, washed with water (3×10 mL), dried over anhydrous Na₂SO₄, and filtered. After filtration, the filtrate was concentrated under reduced pressure to give a residue. The residue was purified by silica gel column chromatography, eluted with EA (100%) to afford tert-butyl 4-[7-({8-fluoro-2-methylimidazo[1,2-a]pyridin-6-yl}carbamoyl)-2-(oxetan-3-ylmethyl)indazol-4-yl]piperazine-1-carboxylate (65 mg, 52%) as a solid. LCMS (ES, m/z): 423.2 [M+H]⁺.

### Synthesis of Compound 338

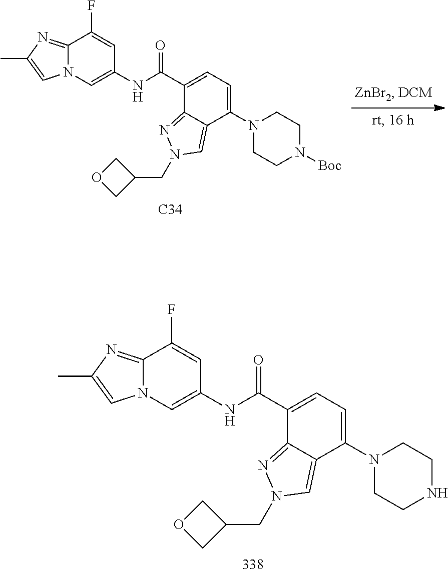 [SMILES: Cc1cn2cc(NC(=O)c3ccc(N4CCN(C(=O)OC(C)(C)C)CC4)c4cn(CC5COC5)nc34)cc(F)c2n1 ; "C34" ; Cc1cn2cc(NC(=O)c3ccc(N4CCNCC4)c4cn(CC5COC5)nc34)cc(F)c2n1 ; "338"]

To a stirred solution of tert-butyl 4-[7-({8-fluoro-2-methylimidazo[1,2-a]pyridin-6-yl}carbamoyl)-2-(oxetan-3-ylmethyl)indazol-4-yl]piperazine-1-carboxylate (20 mg, 0.035 mmol, 1.0 equiv) in DCM (0.5 mL) was added ZnBr₂ (79.91 mg, 0.350 mmol, 10 equiv) at room temperature. The resulting mixture was stirred for 16 h at room temperature, then diluted with water (2 mL) and extracted with CH₂Cl₂ (2×2 mL). The organic layers were combined, dried over anhydrous Na₂SO₄, and filtered. After filtration, the filtrate was concentrated under reduced pressure to give a residue. The residue was purified by Prep-HPLC (Condition 2, Gradient 2) to afford N-{8-fluoro-2-methylimidazo[1,2-a]pyridin-6-yl}-2-(oxetan-3-ylmethyl)-4-(piperazin-1-yl)indazole-7-carboxamide (5.6 mg, 34%) as a solid. LCMS (ES, m/z): 403.2 [M+H]⁺. ¹H NMR (400 MHz, Methanol-d₄) δ 9.02 (s, 1H), 8.55 (s, 1H), 8.06 (d, J=8.0 Hz, 1H), 7.69-7.63 (m, 1H), 7.14 (d, J=11.8 Hz, 1H), 6.50 (d, J=8.2 Hz, 1H), 4.95-4.82 (m, 4H), 4.71 (t, J=6.1 Hz, 2H), 3.82-3.72 (m, 1H), 3.42 (t, J=4.9 Hz, 4H), 3.07 (m, 4H), 2.42 (s, 3H)).

### Example 54: Synthesis of Compound 317

### Synthesis of Intermediate C35

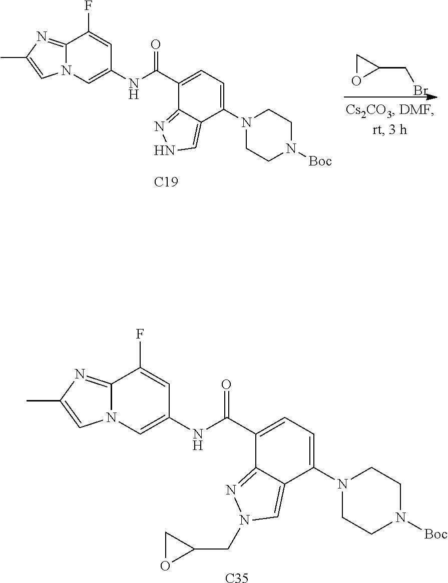 [SMILES: Cc1cn2cc(NC(=O)c3ccc(N4CCN(C(=O)OC(C)(C)C)CC4)c4c[nH]nc34)cc(F)c2n1 ; BrCC1CO1 ; "C19" ; Cc1cn2cc(NC(=O)c3ccc(N4CCN(C(=O)OC(C)(C)C)CC4)c4cn(CC5CO5)nc34)cc(F)c2n1 ; "C35"]

To a stirred mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.20 mmol, 1.0 equiv) and epibromohydrin (41.6 mg, 0.30 mmol, 1.5 equiv) in DMF (1 mL) was added Cs₂CO₃ (198.0 mg, 0.60 mmol, 3.0 equiv) at room temperature. The resulting mixture was stirred for 3 h at room temperature, then diluted with water (2 mL) and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[7-({8-fluoro-2-methylimidazo[1,2-a]pyridin-6-yl}carbamoyl)-2-(oxiran-2-ylmethyl)indazol-4-yl]piperazine-1-carboxylate (55 mg, 49%) as a solid. LCMS (ES, m/z): 550 [M+H]⁺.

### Synthesis of Compound 317

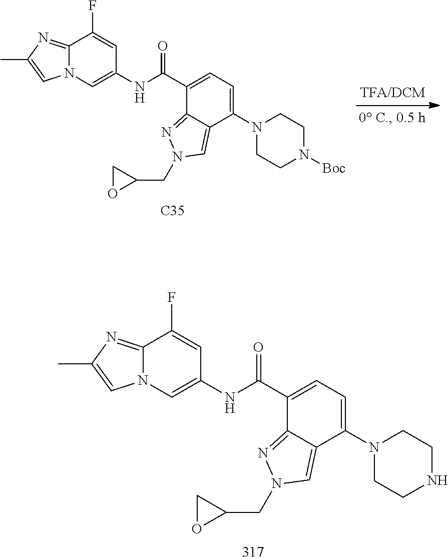 [SMILES: Cc1cn2cc(NC(=O)c3ccc(N4CCN(C(=O)OC(C)(C)C)CC4)c4cn(CC5CO5)nc34)cc(F)c2n1 ; "C35" ; Cc1cn2cc(NC(=O)c3ccc(N4CCNCC4)c4cn(CC5CO5)nc34)cc(F)c2n1 ; "317"]

A solution of tert-butyl 4-[7-({8-fluoro-2-methylimidazo[1,2-a]pyridin-6-yl}carbamoyl)-2-(oxiran-2-ylmethyl)indazol-4-yl]piperazine-1-carboxylate (55 mg, 0.10 mmol, 1.0 equiv) in DCM (0.5 mL) was treated with TFA (0.1 mL) at 0° C. The resulting mixture was stirred for 30 min at 0° C., then concentrated under reduced pressure to give a residue. The residue was purified by Prep-HPLC (Condition 2, Gradient 3) to afford N-{8-fluoro-2-methylimidazo[1,2-a]pyridin-6-yl}-2-(oxiran-2-ylmethyl)-4-(piperazin-1-yl)indazole-7-carboxamide (2 mg, 4%) as a solid. LCMS (ES, m/z): 450 [M+H]⁺.

### Example 55: Synthesis of Compound 339

### Synthesis of Intermediate C21

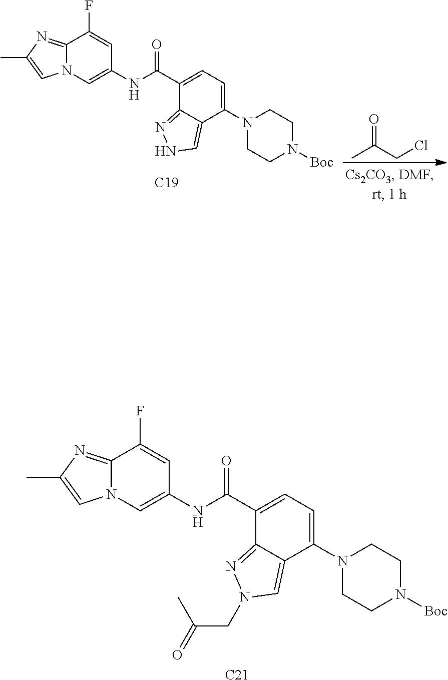

To a stirred mixture of tert-butyl 4-[7-({8-fluoro-2-methylimidazo[1,2-a]pyridin-6-yl}carbamoyl)-2H-indazol-4-yl]piperazine-1-carboxylate (300.0 mg, 0.61 mmol, 1.0 equiv) and 1-chloropropan-2-one (67.4 mg, 0.73 mmol, 1.2 equiv) in DMF (3 mL) was added Cs₂CO₃ (594.1 mg, 1.82 mmol, 3.0 equiv) at room temperature. The resulting mixture was stirred for 1 h at room temperature, then diluted with water and extracted with ethyl acetate (3×6 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with PE/EA (1:3) to afford tert-butyl 4-[7-({8-fluoro-2-methylimidazo[1,2-a]pyridin-6-yl}carbamoyl)-2-(2-oxopropyl)indazol-4-yl]piperazine-1-carboxylate (135 mg, 40%) as a solid. LCMS (ES, m/z): 550 [M+H]⁺.

### Synthesis of Compound 339

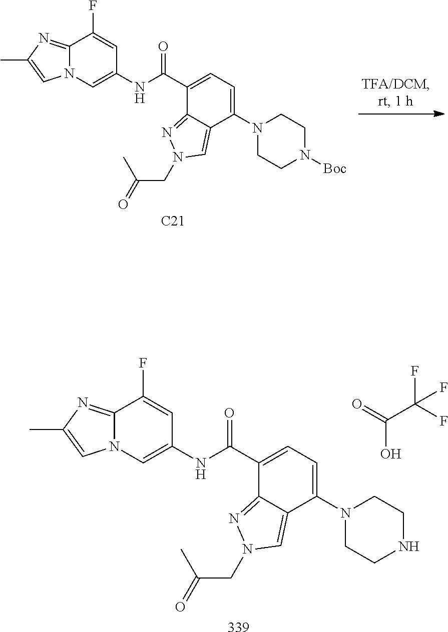

A mixture of tert-butyl 4-[7-({8-fluoro-2-methylimidazo[1,2-a]pyridin-6-yl}carbamoyl)-2-(2-oxopropyl)indazol-4-yl]piperazine-1-carboxylate (50 mg, 0.09 mmol, 1 equiv) in DCM (0.5 mL) and TFA (0.5 mL) was stirred for 1 h at room temperature. The resulting mixture was concentrated under reduced pressure to give a residue. The residue was purified by Prep-HPLC (Condition 3, Gradient 3) to afford N-{8-fluoro-2-methylimidazo[1,2-a]pyridin-6-yl}-2-(2-oxopropyl)-4-(piperazin-1-yl)indazole-7-carboxamide trifluoroacetic acid salt (21.8 mg, 44%) as a solid. LCMS (ES, m/z): 450 [M+H]⁺. ¹H NMR (400 MHz, Methanol-d₄) δ 11.42 (s, 1H), 9.48 (d, J=1.7 Hz, 1H), 8.62 (s, 1H), 8.20 (d, J=8.0 Hz, 1H), 8.03 (d, J=1.9 Hz, 1H), 7.91 (dd, J=11.5, 1.5 Hz, 1H), 6.71 (d, J=8.0 Hz, 1H), 5.65 (s, 2H), 3.71 (dd, J=6.7, 3.8 Hz, 4H), 3.52 (dd, J=6.7, 3.7 Hz, 4H), 2.63-2.49 (m, 3H), 2.36 (s, 3H).

### Example 56: Synthesis of Compound 308

### Synthesis of Intermediate C36

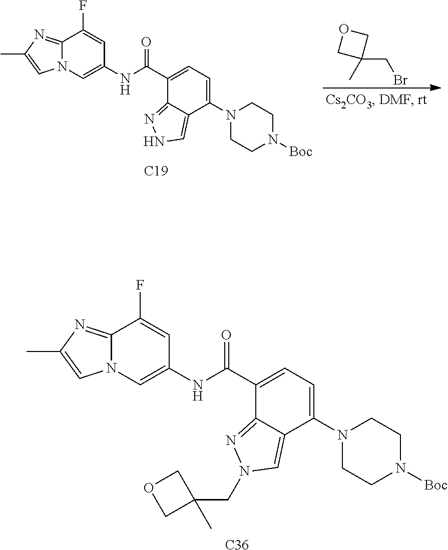

To a stirred mixture of tert-butyl 4-[7-({8-fluoro-2-methylimidazo[1,2-a]pyridin-6-yl}carbamoyl)-2H-indazol-4-yl]piperazine-1-carboxylate (100 mg, 0.20 mmol, 1 equiv) and 3-(bromomethyl)-3-methyloxetane (50.1 mg, 0.30 mmol, 1.5 equiv) in DMF (1 mL) was added Cs₂CO₃ (198.0 mg, 0.60 mmol, 3 equiv) at room temperature. The resulting mixture was stirred for 3 h at room temperature, then diluted with water (2 mL) and extracted with ethyl acetate (3×2 mL). The organic layers were combined, washed with brine (1×2 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[7-({8-fluoro-2-methylimidazo[1,2-a]pyridin-6-yl}carbamoyl)-2-[(3-methyloxetan-3-yl)methyl] indazol-4-yl]piperazine-1-carboxylate (42 mg, 36%) as a solid. LCMS (ES, m/z): 578 [M+H]⁺.

### Synthesis of Compound 308

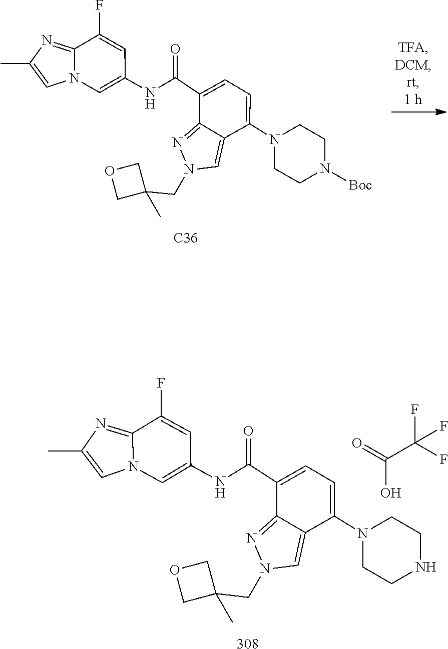

To a stirred mixture of tert-butyl 4-[7-({8-fluoro-2-methylimidazo[1,2-a]pyridin-6-yl}carbamoyl)-2-[(3-methyloxetan-3-yl)methyl]indazol-4-yl]piperazine-1-carboxylate (42 mg, 0.07 mmol, 1.0 equiv) in DCM (0.4 mL) was added TFA (0.4 mL) at room temperature. The resulting mixture was stirred for 1 h at room temperature, then concentrated under reduced pressure to give a residue. The residue was purified by Prep-HPLC (Condition 3, Gradient 8) to afford N-{8-fluoro-2-methylimidazo[1,2-a]pyridin-6-yl}-2-[(3-methyloxetan-3-yl)methyl]-4-(piperazin-1-yl)indazole-7-carboxamide trifluoroacetic acid salt (20.1 mg, 48%) as a solid. LCMS (ES, m/z): 478 [M+H]⁺. ¹H NMR (300 MHz, DMSO-d₆) δ 10.87 (s, 1H), 9.63 (s, 1H), 9.20 (d, J=1.6 Hz, 1H), 9.16 (s, 2H), 8.20 (d, J=8.2 Hz, 1H), 8.08 (d, J=2.9 Hz, 1H), 7.53 (d, J=12.3 Hz, 1H), 6.98 (d, J=8.3 Hz, 1H), 4.76 (dd, J=12.3, 5.4 Hz, 2H), 4.56 (dd, J=12.3, 7.5 Hz, 2H), 3.67 (t, J=5.5 Hz, 4H), 3.52 (s, 2H), 3.39 (s, 4H), 2.40 (s, 3H), 1.32 (s, 3H).

### Example 57: Synthesis of Compound 331

### Synthesis of Intermediate C37

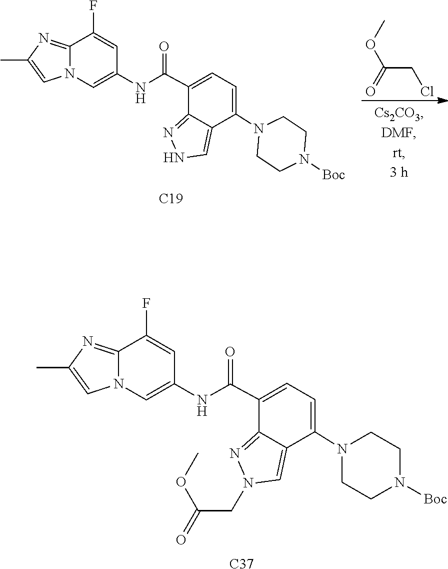

To a stirred mixture of tert-butyl 4-[7-({8-fluoro-2-methylimidazo[1,2-a]pyridin-6-yl}carbamoyl)-2H-indazol-4-yl]piperazine-1-carboxylate (120.0 mg, 0.24 mmol, 1.0 equiv) and methyl chloroacetate (39.5 mg, 0.36 mmol, 1.5 equiv) in DMF (1.2 mL) was added Cs₂CO₃ (237.6 mg, 0.73 mmol, 3.0 equiv) at room temperature. The resulting mixture was stirred for 3 h at room temperature, then diluted with water (3 mL) and extracted with ethyl acetate (3×2 mL). The organic layers were combined, washed with brine (1×2 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[7-({8-fluoro-2-methylimidazo[1,2-a]pyridin-6-yl}carbamoyl)-2-(2-methoxy-2-oxoethyl)indazol-4-yl]piperazine-1-carboxylate (48 mg, 35%) as a solid. LCMS (ES, m/z): 566 [M+H]⁺.

### Synthesis of Intermediate C38

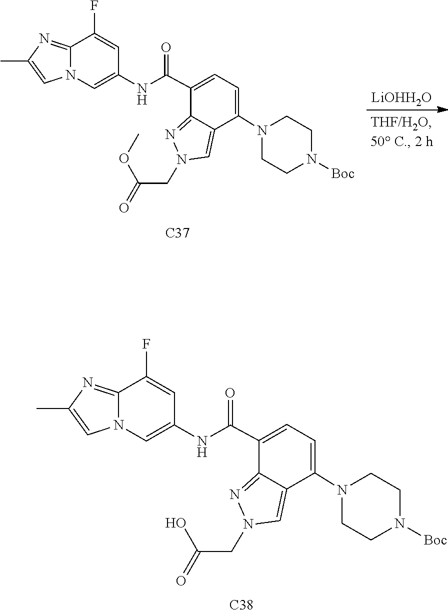 [SMILES: COC(=O)Cn1cc2c(N3CCN(C(=O)OC(C)(C)C)CC3)ccc(C(=O)Nc3cc(F)c4nc(C)cn4c3)c2n1 ; "LiOHH2O" ; "C37" ; Cc1cn2cc(NC(=O)c3ccc(N4CCN(C(=O)OC(C)(C)C)CC4)c4cn(CC(=O)O)nc34)cc(F)c2n1 ; "C38"]

A solution of tert-butyl 4-[7-({8-fluoro-2-methylimidazo[1,2-a]pyridin-6-yl}carbamoyl)-2-(2-methoxy-2-oxoethyl)indazol-4-yl]piperazine-1-carboxylate (48.0 mg, 0.08 mmol, 1.0 equiv) in tetrahydrofuran (0.5 mL) was treated with LiOH·H₂O (10.1 mg, 0.42 mmol, 5.0 equiv) in water (0.5 mL) at room temperature. The resulting mixture was stirred for 2 h at 50° C. under nitrogen atmosphere, then cooled to 0° C., acidified to pH 4 with HCl (1 M), and extracted with ethyl acetate (2×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford {4-[4-(tert-butoxycarbonyl)piperazin-1-yl]-7-({8-fluoro-2-methylimidazo[1,2-a]pyridin-6-yl}carbamoyl)indazol-2-yl}acetic acid (34 mg, 73%) as a solid. LCMS (ES, m/z): 552 [M+H]⁺.

### Synthesis of Compound 331

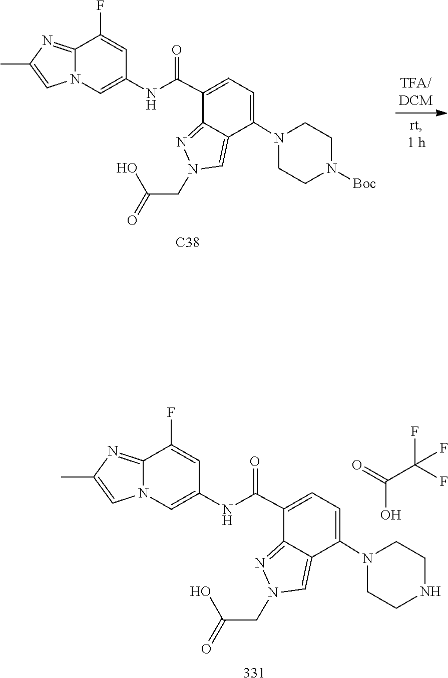

A solution of {4-[4-(tert-butoxycarbonyl)piperazin-1-yl]-7-({8-fluoro-2-methylimidazo[1,2-a]pyridin-6-yl}carbamoyl)indazol-2-yl}acetic acid (34 mg, 0.06 mmol, 1.0 equiv) in DCM (0.4 mL) was treated with TFA (0.4 mL) at room temperature. The resulting mixture was stirred for 1 h at room temperature, then concentrated under reduced pressure to give a residue. The residue was purified by Prep-HPLC (Condition 3, Gradient 9) to afford [7-({8-fluoro-2-methylimidazo[1,2-a]pyridin-6-yl}carbamoyl)-4-(piperazin-1-yl)indazol-2-yl]acetic acid trifluoroacetic acid salt (2.5 mg, 7%) as a solid. LCMS (ES, m/z): 452 [M+H]⁺. ¹H NMR (300 MHz, DMSO-d₆) δ 11.17 (s, 1H), 9.39 (s, 1H), 8.96 (s, 1H), 8.89 (s, 2H), 8.15-8.01 (m, 2H), 7.66 (d, J=12.2 Hz, 1H), 6.64 (d, J=8.1 Hz, 1H), 5.54 (s, 2H), 3.60 (d, J=5.7 Hz, 4H), 3.37 (s, 4H), 2.40 (s, 3H).

### Example 58: Synthesis of Compound 309

### Synthesis of Intermediate C39

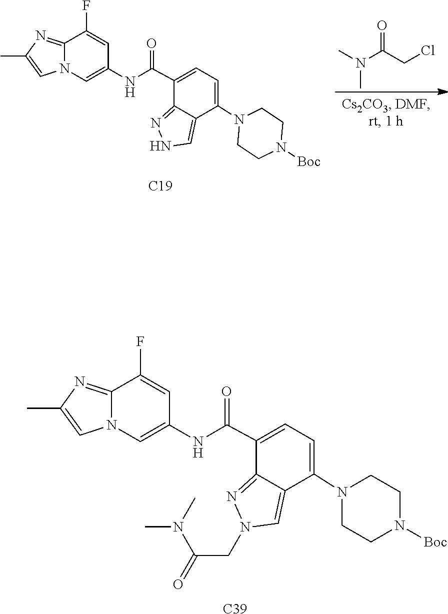

To a stirred mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.20 mmol, 1.0 equiv) and 2-chloro-N,N-dimethylacetamide (36.9 mg, 0.30 mmol, 1.5 equiv) in DMF (1 mL) was added Cs₂CO₃ (198.0 mg, 0.60 mmol, 3.0 equiv) at room temperature. The resulting mixture was stirred for 3 h at room temperature, then diluted with water (3 mL) and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-{2-[(dimethylcarbamoyl)methyl]-7-({8-fluoro-2-methylimidazo[1,2-a]pyridin-6-yl}carbamoyl)indazol-4-yl}piperazine-1-carboxylate (45 mg, 38%) as a solid. LCMS (ES, m/z): 579 [M+H]⁺.

### Synthesis of Compound 309

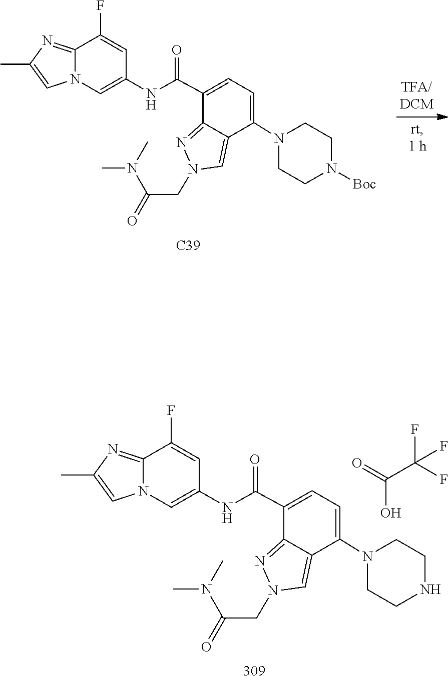

To a stirred solution of tert-butyl 4-{2-[(dimethylcarbamoyl)methyl]-7-({8-fluoro-2-methylimidazo[1,2-a]pyridin-6-yl}carbamoyl)indazol-4-yl}piperazine-1-carboxylate (47 mg, 0.081 mmol, 1 equiv) in DCM (0.4 mL) was added TFA (0.4 mL) at room temperature. The resulting mixture was stirred for 1 h at room temperature, then concentrated under reduced pressure to give a residue. The residue was purified by Prep-HPLC (Condition 3, Gradient 10) to afford 2-[(dimethylcarbamoyl)methyl]-N-{8-fluoro-2-methylimidazo[1,2-a]pyridin-6-yl}-4-(piperazin-1-yl)indazole-7-carboxamide trifluoroacetic acid salt (24.1 mg, 62%) as a solid. LCMS (ES, m/z): 479 [M+H]⁺. ¹H NMR (300 MHz, DMSO-d6) δ 11.26 (s, 1H), 9.51 (d, J=1.6 Hz, 1H), 9.14 (s, 2H), 8.86 (s, 1H), 8.15 (d, J=2.6 Hz, 1H), 8.05 (d, J=8.0 Hz, 1H), 7.82 (d, J=11.8 Hz, 1H), 6.63 (d, J=8.1 Hz, 1H), 5.68 (s, 2H), 3.62 (t, J=5.1 Hz, 4H), 3.36 (s, 4H), 3.18 (s, 3H), 2.93 (s, 3H), 2.47 (s, 3H).

### Example 59: Synthesis of Compound 355

### Synthesis of Intermediate C40

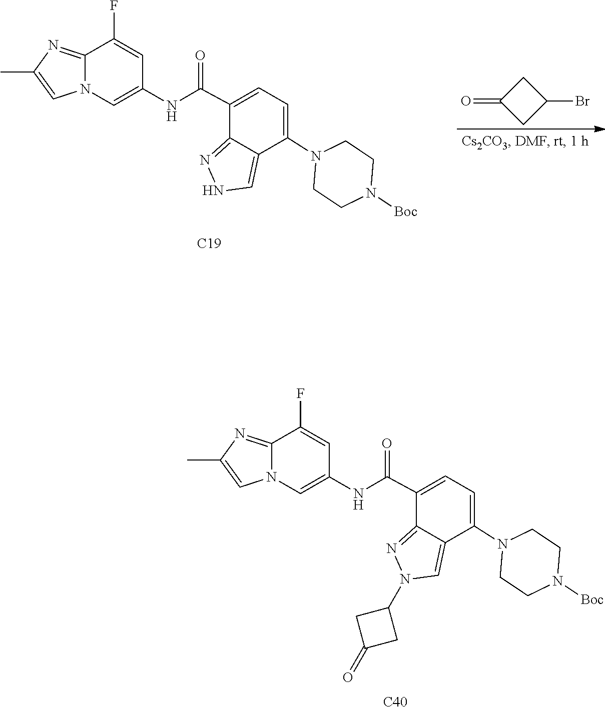 [SMILES: Cc1cn2cc(NC(=O)c3ccc(N4CCN(C(=O)OC(C)(C)C)CC4)c4c[nH]nc34)cc(F)c2n1 ; O=C1CC(Br)C1 ; "C19" ; Cc1cn2cc(NC(=O)c3ccc(N4CCN(C(=O)OC(C)(C)C)CC4)c4cn(C5CC(=O)C5)nc34)cc(F)c2n1 ; "C40"]

A mixture of tert-butyl 4-[7-({8-fluoro-2-methylimidazo[1,2-a]pyridin-6-yl}carbamoyl)-2H-indazol-4-yl]piperazine-1-carboxylate (150.0 mg, 0.304 mmol, 1.0 equiv), dimethylformamide (3 mL), caesio methaneperoxoate caesium (297.9 mg, 0.912 mmol, 3.0 equiv) and 3-bromocyclobutan-1-one (58.8 mg, 0.395 mmol, 1.3 equiv) was stirred for 1 h at room temperature. The reaction was quenched with water (10 mL) and extracted with ethyl acetate (2×10 mL). The organic layers were combined, dried over Na₂SO₄, and filtered. The filtrate was concentrated under reduced pressure to give a residue. The residue was purified by silica gel column chromatography, eluted with CH₂Cl₂/MeOH (40:1) to afford tert-butyl 4-[7-({8-fluoro-2-methylimidazo[1,2-a]pyridin-6-yl}carbamoyl)-2-(3-oxocyclobutyl)indazol-4-yl] piperazine-1-carboxylate (70 mg, 37%) as a solid. LCMS (ES, m/z): 562 [M+H]⁺.

### Synthesis of Intermediate C41

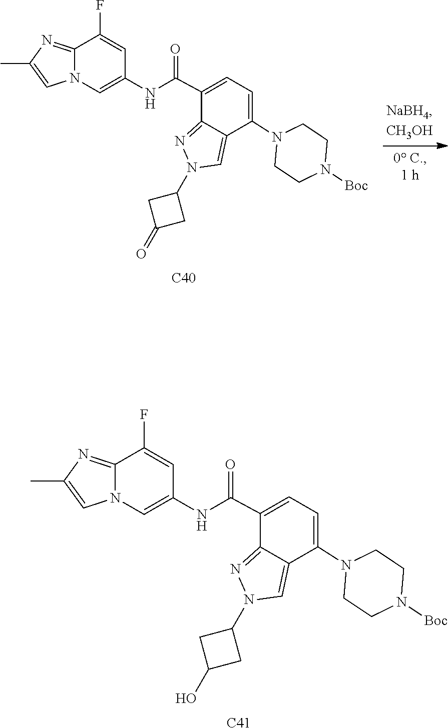 [SMILES: Cc1cn2cc(NC(=O)c3ccc(N4CCN(C(=O)OC(C)(C)C)CC4)c4cn(C5CC(=O)C5)nc34)cc(F)c2n1 ; "C40" ; Cc1cn2cc(NC(=O)c3ccc(N4CCN(C(=O)OC(C)(C)C)CC4)c4cn(C5CC(O)C5)nc34)cc(F)c2n1 ; "C41"]

A mixture of tert-butyl 4-[7-({8-fluoro-2-methylimidazo[1,2-a]pyridin-6-yl}carbamoyl)-2-(3-oxocyclobutyl)indazol-4-yl]piperazine-1-carboxylate (50.0 mg, 0.089 mmol, 1.0 equiv), methanol (1 mL), and NaBH₄ (6.7 mg, 0.178 mmol, 2.0 equiv) was stirred for 1 h at 0° C. under nitrogen atmosphere. The reaction was quenched with water (5 mL) and extracted with ethyl acetate (2×5 mL). The organic layers were combined, dried over Na₂SO₄, and filtered. The filtrate was concentrated under reduced pressure to give a residue. The residue was purified by silica gel column chromatography, eluted with CH₂Cl₂/MeOH (30:1) to afford tert-butyl 4-[7-({8-fluoro-2-methylimidazo[1,2-a]pyridin-6-yl}carbamoyl)-2-(3-hydroxycyclobutyl)indazol-4-yl]piperazine-1-carboxylate (45 mg, 81%) as a solid. LCMS (ES, m/z): 564 [M+H]⁺.

### Synthesis of Compound 355

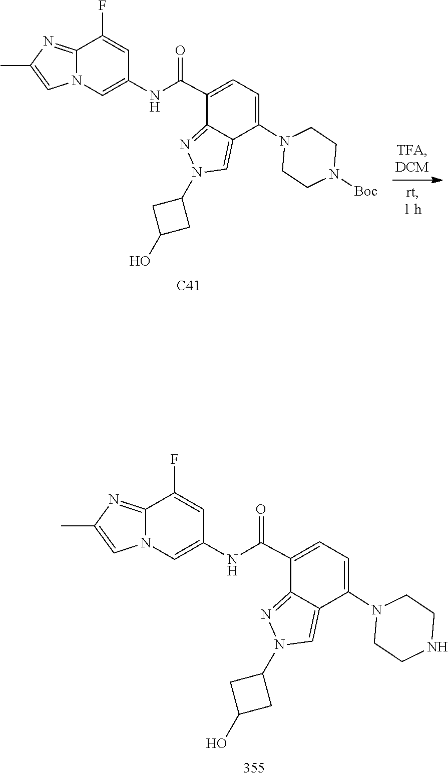

A mixture of tert-butyl 4-[7-({8-fluoro-2-methylimidazo[1,2-a]pyridin-6-yl}carbamoyl)-2-(3-hydroxycyclobutyl)indazol-4-yl]piperazine-1-carboxylate (40.0 mg, 0.071 mmol, 1.0 equiv), DCM (1 mL) and trifluoroacetaldehyde (4 mL) was stirred for 1 h at room temperature. The resulting mixture was concentrated under reduced pressure to give a residue. The residue was purified by Prep-HPLC (Condition 4, Gradient 1) to afford N-{8-fluoro-2-methylimidazo[1,2-a]pyridin-6-yl}-2-(3-hydroxycyclobutyl)-4-(piperazin-1-yl)indazole-7-carboxamide (5 mg, 15.00%) as a solid. LCMS (ES, m/z): 463 [M+H]⁺. ¹H NMR (300 MHz, DMSO-d₆) δ 11.27 (s, 1H), 9.28 (d, J=1.6 Hz, 1H), 8.85 (s, 1H), 7.99 (d, J=8.1 Hz, 1H), 7.95-7.88 (m, 1H), 7.27 (dd, J=12.3, 1.7 Hz, 1H), 6.50 (d, J=8.2 Hz, 1H), 5.53 (d, J=6.1 Hz, 1H), 4.87 (t, J=8.0 Hz, 1H), 4.14 (q, J=7.0 Hz, 1H), 3.37-3.36 (m, 4H), 2.92-2.90 (m, 6H), 2.72-2.59 (m, 2H), 2.35 (s, 3H).

### Example 60: Synthesis of Compound 311

### Synthesis of Intermediate C42

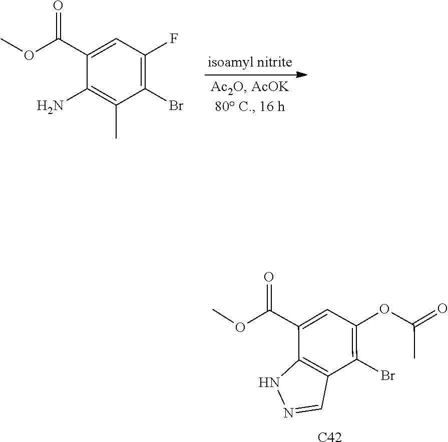 [SMILES: COC(=O)c1cc(F)c(Br)c(C)c1N ; COC(=O)c1cc(OC(C)=O)c(Br)c2cn[nH]c12 ; "C42"]

A mixture of methyl 2-amino-4-bromo-5-fluoro-3-methylbenzoate (1.2 g, 4.579 mmol, 1.0 equiv) and Ac₂O (0.6 g, 5.953 mmol, 1.3 equiv) was stirred for 1 h at 25° C. To the reaction mixture was added potassium acetate (0.13 g, 1.374 mmol, 0.3 equiv) and isoamyl nitrite (1.1 g, 10.074 mmol, 2.2 equiv). The resulting mixture was stirred for an additional 2 h at 80° C., then quenched with water (100 mL) and extracted with CH₂Cl₂ (2×100 mL). The organic layers were combined, dried over Na₂SO₄, and filtered. The filtrate was concentrated under reduced pressure to give a residue. The residue was purified by silica gel column chromatography, eluted with PE/EA (1:1) to afford methyl methyl 5-(acetyloxy)-4-bromo-1H-indazole-7-carboxylate (800 g, 50%) as a solid. LCMS (ES, m/z): 313 [M+H]⁺.

### Synthesis of Intermediate C43

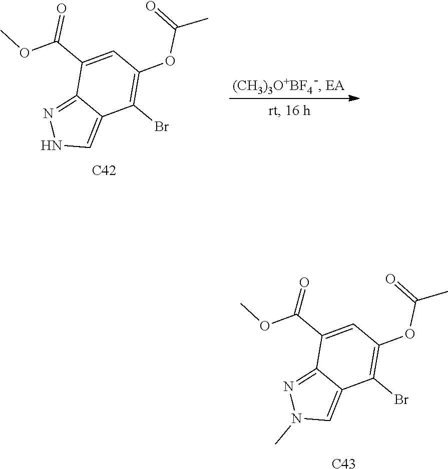 [SMILES: COC(=O)c1cc(OC(C)=O)c(Br)c2c[nH]nc12 ; "C42" ; COC(=O)c1cc(OC(C)=O)c(Br)c2cn(C)nc12 ; "C43"]

A mixture of methyl 5-(acetyloxy)-4-bromo-2H-indazole-7-carboxylate (0.8 g, 2.555 mmol, 1.0 equiv), ethyl acetate (15 mL), and (CH₃)₃O⁺BF₄⁻ (1.5 g, 10.220 mmol, 4.0 equiv) was stirred for 16 h at room temperature. The reaction mixture was quenched with water (50 mL) and extracted with ethyl acetate (2×50 mL). The organic layer were combined, dried over Na₂SO₄, and concentrated under reduced pressure to give a residue. The residue was purified by silica gel column chromatography, eluted with PE/EA (1:1) to afford methyl 5-(acetyloxy)-4-bromo-2-methylindazole-7-carboxylate (780 mg, 84%) as a solid. LCMS (ES, m/z): 327 [M+H]⁺.

### Synthesis of Intermediate C44

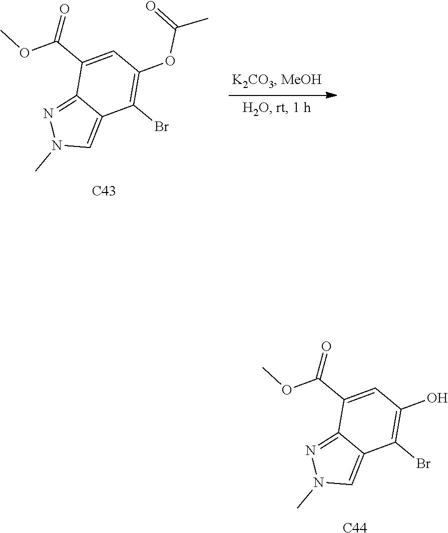 [SMILES: COC(=O)c1cc(OC(C)=O)c(Br)c2cn(C)nc12 ; "C43" ; COC(=O)c1cc(O)c(Br)c2cn(C)nc12 ; "C44"]

A mixture of methyl 5-(acetyloxy)-4-bromo-2-methylindazole-7-carboxylate (0.8 g, 2.445 mmol, 1.0 equiv), methanol (10 mL), water (5 mL), and K₂CO₃ (1.0 g, 7.335 mmol, 3.0 equiv) was stirred for 1 h at room temperature. The reaction mixture was quenched with water (100 mL) and extracted with ethyl acetate (2×100 mL). The organic layers were combined, dried over Na₂SO₄, and filtered. The filtrate was concentrated under reduced pressure to give a residue. The residue was purified by silica gel column chromatography, eluted with PE/EA (1:1) to afford methyl 4-bromo-5-hydroxy-2-methylindazole-7-carboxylate (410 mg, 53%) as a solid. LCMS (ES, m/z): 285 [M+H]⁺.

### Synthesis of Intermediate C45

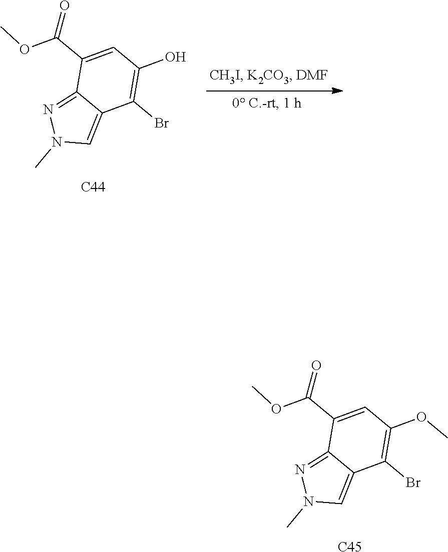

A mixture of methyl 4-bromo-5-hydroxy-2-methylindazole-7-carboxylate (0.8 g, 2.806 mmol, 1.0 equiv), K₂CO₃ (0.8 g, 5.612 mmol, 2.0 equiv), DMF (15 mL), and methyl iodide (0.8 g, 5.612 mmol, 2.0 equiv) was stirred for 1 h at room temperature. The reaction mixture was quenched with water (100 mL) and extracted with ethyl acetate (2×100 mL). The organic layers were combined, dried over Na₂SO₄, and filtered. The filtrate was concentrated under reduced pressure to give a residue. The residue was purified by silica gel column chromatography, eluted with PE/EA (1:1) to afford methyl 4-bromo-5-methoxy-2-methylindazole-7-carboxylate (750 mg, 79%) as a solid. LCMS (ES, m/z): 299 [M+H]⁺.

### Synthesis of Intermediate C46

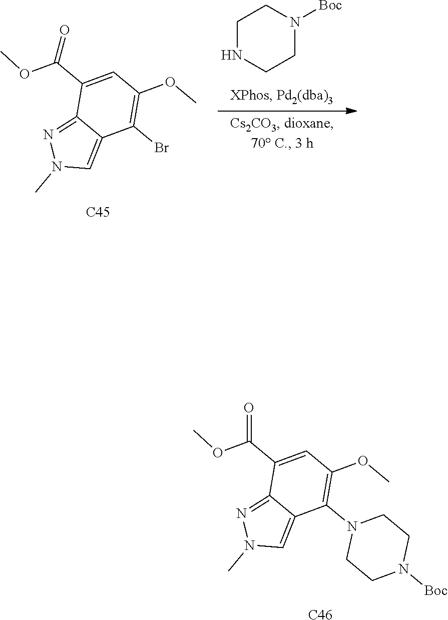

A mixture of methyl 4-bromo-5-methoxy-2-methylindazole-7-carboxylate (0.7 g, 2.340 mmol, 1.0 equiv), Cs₂CO₃ (1.5 g, 4.680 mmol, 2.0 equiv), tert-butyl piperazine-1-carboxylate (0.8 g, 4.680 mmol, 2.0 equiv), XPhos (0.2 g, 0.468 mmol, 0.2 equiv), Pd₂(dba)₃ (0.2 g, 0.234 mmol, 0.1 equiv), and dioxane (10 mL) was stirred for 3 h at 70° C. under nitrogen atmosphere. The resulting mixture was concentrated under reduced pressure to give a residue. The residue was purified by silica gel column chromatography, eluted with PE/EA (1:1) to afford methyl 4-[4-(tert-butoxycarbonyl)piperazin-1-yl]-5-methoxy-2-methylindazole-7-carboxylate (0.9 g, 86%) as a solid. LCMS (ES, m/z): 405 [M+H]⁺.

### Synthesis of Intermediate C47

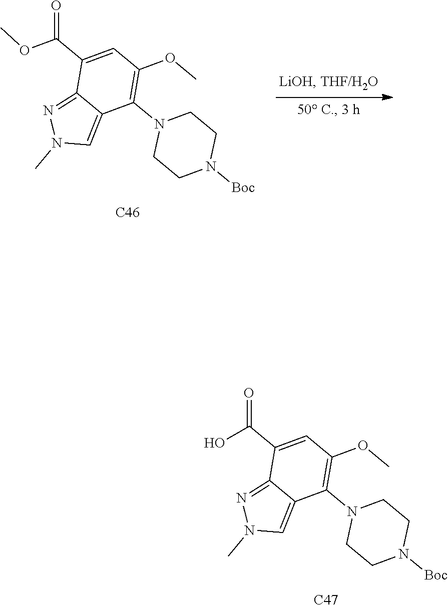 [SMILES: COC(=O)c1cc(OC)c(N2CCN(C(=O)OC(C)(C)C)CC2)c2cn(C)nc12 ; "C46" ; COc1cc(C(=O)O)c2nn(C)cc2c1N1CCN(C(=O)OC(C)(C)C)CC1 ; "C47"]

A mixture of methyl 4-[4-(tert-butoxycarbonyl)piperazin-1-yl]-5-methoxy-2-methylindazole-7-carboxylate (0.9 g, 2.225 mmol, 1.0 equiv), THE (10 mL), water (5 mL), and LiOH (0.5 g, 22.250 mmol, 10.0 equiv) was stirred for 3 h at 50° C. The resulting mixture was diluted with water (100 mL), acidified to pH 5 with HCl (aq.), and extracted with ethyl acetate (2×100 mL). The organic layers were combined, dried over Na₂SO₄, and filtered. The filtrate was concentrated under reduced pressure to afford 4-[4-(tert-butoxycarbonyl)piperazin-1-yl]-5-methoxy-2-methylindazole-7-carboxylic acid (0.63 g, 73%) as a solid. LCMS (ES, m/z): 391 [M+H]⁺.

### Synthesis of Intermediate C48

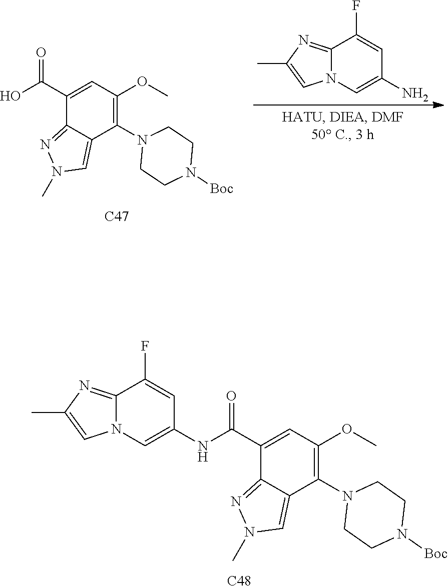 [SMILES: COc1cc(C(=O)O)c2nn(C)cc2c1N1CCN(C(=O)OC(C)(C)C)CC1 ; Cc1cn2cc(N)cc(F)c2n1 ; "C47" ; COc1cc(C(=O)Nc2cc(F)c3nc(C)cn3c2)c2nn(C)cc2c1N1CCN(C(=O)OC(C)(C)C)CC1 ; "C48"]

A mixture of 4-[4-(tert-butoxycarbonyl)piperazin-1-yl]-5-methoxy-2-methylindazole-7-carboxylic acid (250.0 mg, 0.640 mmol, 1.0 equiv), 8-fluoro-2-methylimidazo[1,2-a]pyridin-6-amine (137.4 mg, 0.832 mmol, 1.3 equiv), HATU (486.9 mg, 1.280 mmol, 2.0 equiv), DIEA (248.2 mg, 1.920 mmol, 3.0 equiv) and DMF (6 mL) was stirred for 3 h at 50° C. The reaction mixture was diluted with water (50 mL) and extracted with ethyl acetate (2×50 mL). The organic layers were combined, dried over Na₂SO₄, and filtered. The filtrate was concentrated under reduced pressure to give a residue. The residue was purified by silica gel column chromatography, eluted with PE/EA (1:1) to afford tert-butyl 4-[7-({8-fluoro-2-methylimidazo[1,2-a]pyridin-6-yl}carbamoyl)-5-methoxy-2-methylindazol-4-yl]piperazine-1-carboxylate (350 mg, 92%) as a solid. LCMS (ES, m/z): 538 [M+H]⁺.

### Synthesis of Compound 311

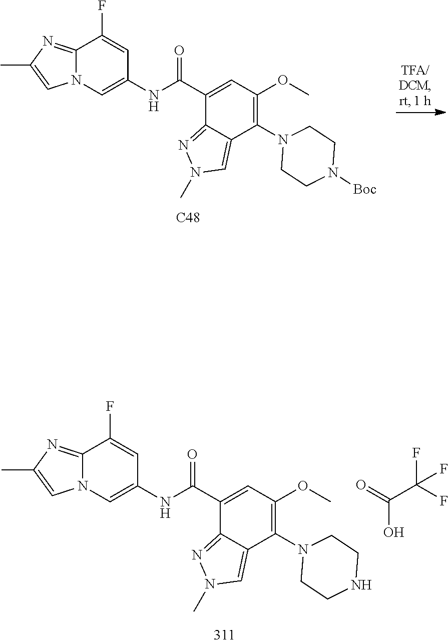

A mixture of tert-butyl 4-[7-({8-fluoro-2-methylimidazo[1,2-a]pyridin-6-yl}carbamoyl)-5-methoxy-2-methylindazol-4-yl]piperazine-1-carboxylate (90.0 mg, 0.167 mmol, 1.0 equiv), DCM (1 m L), and TFA (1 mL) was stirred for 1 h at room temperature. The resulting mixture was concentrated under reduced pressure to give a residue. The residue was purified by Prep-HPLC (Condition 3, Gradient 2) to afford N-{8-fluoro-2-methylimidazo[1,2-a]pyridin-6-yl}-5-methoxy-2-methyl-4-(piperazin-1-yl)indazole-7-carboxamide trifluoroacetic acid salt (32 mg, 42%) as a solid. LC MS (ES, m/z): 526 [M+H]⁺. ¹H NMR (300 MHz, DMSO-d₆) δ 11.24 (m, 1H), 9.44 (d, J=1.5 Hz, 1H), 9.18 (s, 2H), 8.79 (s, 1H), 8.15 (d, J=2.6 Hz, 1H), 7.91 (s, 1H), 7.85 (d, J=12.3 Hz, 1H), 4.33 (d, J=4.4 Hz, 3H), 3.88 (s, 3H), 3.63 (t, J=5.0 Hz, 4H), 3.28-3.27 (m, 4H), 2.49 (s, 3H).

### Example 61: Synthesis of Compound 312

### Synthesis of Intermediate C49

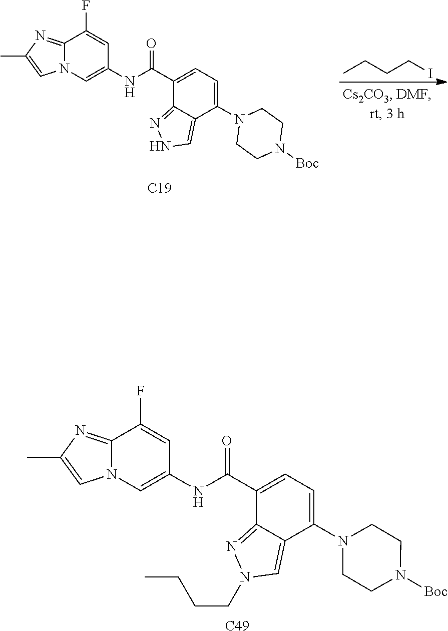

To a stirred mixture of tert-butyl 4-[7-({8-fluoro-2-methylimidazo[1,2-a]pyridin-6-yl}carbamoyl)-2H-indazol-4-yl]piperazine-1-carboxylate (100 mg, 0.20 mmol, 1 equiv) and butyl iodide (55.9 mg, 0.30 mmol, 1.5 equiv) in DMF (1 mL) was added Cs₂CO₃ (198.0 mg, 0.60 mmol, 3 equiv) at room temperature. The resulting mixture was stirred for 3 h at room temperature, then diluted with water (3 mL) and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[2-butyl-7-({8-fluoro-2-methylimidazo[1,2-a]pyridin-6-yl}carbamoyl)indazol-4-yl]piperazine-1-carboxylate (42 mg, 38%) as a solid. LCMS (ES, m/z): 550 [M+H]⁺.

### Synthesis of Compound 312

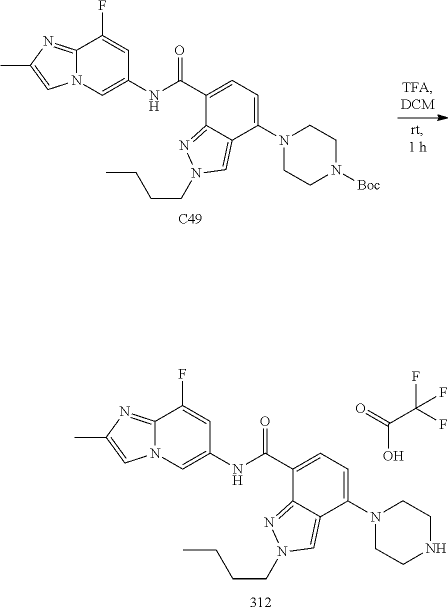

To a stirred solution of tert-butyl 4-[2-butyl-7-({8-fluoro-2-methylimidazo[1,2-a]pyridin-6-yl}carbamoyl)indazol-4-yl]piperazine-1-carboxylate (42 mg, 0.07 mmol, 1 equiv) in DCM (0.4 mL) was added TFA (0.4 mL) at room temperature. The resulting mixture was stirred for 1 h at room temperature, then concentrated under reduced pressure to give a residue. The residue was purified by Prep-HPLC (Condition 3, Gradient 11) to afford 2-butyl-N-{8-fluoro-2-methylimidazo[1,2-a]pyridin-6-yl}-4-(piperazin-1-yl)indazole-7-carboxamide trifluoroacetic acid salt (23.6 mg, 56%) as a solid. LCMS (ES, m/z): 450 [M+H]⁺. ¹H NMR (300 MHz, DMSO-d₆) δ 11.25 (s, 1H), 9.45 (d, J=1.6 Hz, 1H), 9.05 (s, 2H), 8.96 (s, 1H), 8.14 (s, 1H), 8.04 (d, J=8.0 Hz, 1H), 7.73 (d, J=11.9 Hz, 1H), 6.62 (d, J=8.1 Hz, 1H), 4.59 (t, J=7.0 Hz, 2H), 3.61 (s, 4H), 3.36 (s, 4H), 2.46-2.41 (m, 3H), 2.03 (p, J=7.1 Hz, 2H), 1.35 (q, J=7.5 Hz, 2H), 0.97 (t, J=7.4 Hz, 3H).

### Example 62: Synthesis of Compound 313

### Synthesis of Intermediate C50

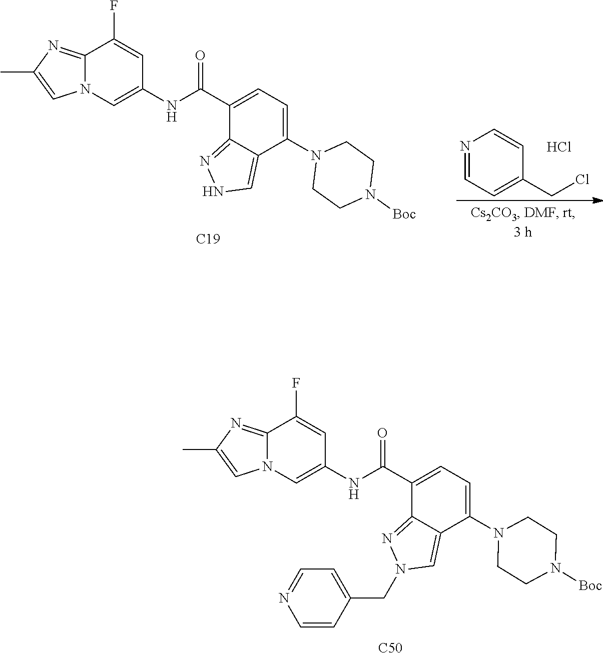 [SMILES: Cc1cn2cc(NC(=O)c3ccc(N4CCN(C(=O)OC(C)(C)C)CC4)c4c[nH]nc34)cc(F)c2n1 ; ClCc1ccncc1 ; Cl ; "C19" ; Cc1cn2cc(NC(=O)c3ccc(N4CCN(C(=O)OC(C)(C)C)CC4)c4cn(Cc5ccncc5)nc34)cc(F)c2n1 ; "C50"]

A mixture of tert-butyl 4-[7-({8-fluoro-2-methylimidazo[1,2-a]pyridin-6-yl} carbamoyl)-2H-indazol-4-yl]piperazine-1-carboxylate (90.0 mg, 0.182 mmol, 1.0 equiv), Cs₂CO₃ (178.8 mg, 0.546 mmol, 3.0 equiv), 4-(chloromethyl)pyridine (25.5 mg, 0.200 mmol, 1.1 equiv), and DMF (2 mL) was stirred for 3 h at room temperature. The reaction was quenched with water (10 mL) and extracted with ethyl acetate (2×10 mL). The organic layers were combined, dried over Na₂SO₄, and filtered. The filtrate was concentrated in vacuo to give a residue. The residue was purified by silica gel column chromatography, eluted with CH₂Cl₂/MeOH (20:1) to afford tert-butyl 4-[7-({8-fluoro-2-methylimidazo[1,2-a]pyridin-6-yl}carbamoyl)-2-(pyridin-4-ylmethyl)indazol-4-yl]piperazine-1-carboxylate (60 mg, 51%) as a solid. LCMS (ES, m/z): 585 [M+H]⁺.

### Synthesis of Compound 313

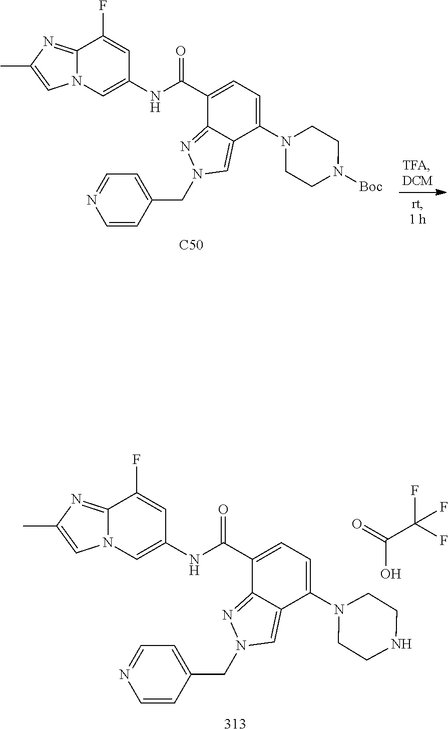

A mixture of tert-butyl 4-[7-({8-fluoro-2-methylimidazo[1,2-a]pyridin-6-yl}carbamoyl)-2-(pyrid in-4-ylmethyl)indazol-4-yl]piperazine-1-carboxylate (70.0 mg, 0.120 mmol, 1.0 equiv), DCM (1 mL), and TFA (1 mL) was stirred for 1 h at room temperature. The resulting mixture was concentrated under reduced pressure to give a residue. The residue was purified by Prep-HPLC (Condition 3, Gradient 2) to afford N-{8-fluoro-2-methylimidazo[1,2-a]pyridin-6-yl}-4-(piperazin-1-yl)-2-(pyridin-4-ylmethyl)indazole-7-carboxamide trifluoroacetic acid salt (22 mg, 36%) as a solid. LCMS (ES, m/z): 485 [M+H]⁺. ¹H NMR (300 MHz, DMSO-d₆) δ 11.19 (s, 1H), 9.48 (d, J=1.5 Hz, 1H), 9.17 (s, 1H), 9.11 (s, 2H), 8.73-8.65 (m, 2H), 8.20-8.13 (m, 1H), 8.07 (d, J=8.0 Hz, 1H), 7.74 (dd, J=11.9, 1.6 Hz, 1H), 7.56-7.48 (m, 2H), 6.66 (d, J=8.1 Hz, 1H), 5.98 (s, 2H), 3.68-3.59 (m, 4H), 3.36-3.35 (m, 4H), 2.45 (s, 3H).

### Example 63: Synthesis of Compound 318

### Synthesis of Intermediate C51

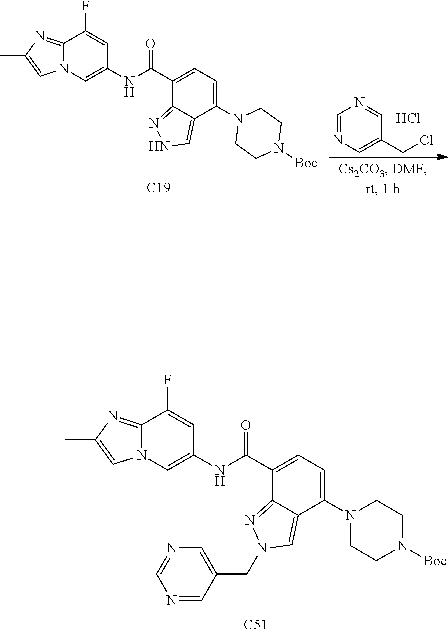 [SMILES: Cc1cn2cc(NC(=O)c3ccc(N4CCN(C(=O)OC(C)(C)C)CC4)c4c[nH]nc34)cc(F)c2n1 ; ClCc1cncnc1 ; Cl ; "C19" ; Cc1cn2cc(NC(=O)c3ccc(N4CCN(C(=O)OC(C)(C)C)CC4)c4cn(Cc5cncnc5)nc34)cc(F)c2n1 ; "C51"]

A mixture of tert-butyl 4-[7-({8-fluoro-2-methylimidazo[1,2-a]pyridin-6-yl}carbamoyl)-2H-indazol-4-yl]piperazine-1-carboxylate (90.0 mg, 0.182 mmol, 1.0 equiv), Cs₂CO₃ (178.8 mg, 0.546 mmol, 3.0 equiv), DMF (2 mL), and 5-(chloromethyl)pyrimidine (28.1 mg, 0.218 mmol, 1.2 equiv) was stirred for 1 h at room temperature. The reaction was quenched with water (20 mL) and extracted with ethyl acetate (2×20 mL). The organic layers were combined, dried over Na₂SO₄, and filtered. The filtrate was concentrated under reduced pressure to give a residue. The residue was purified by silica gel column chromatography, eluted with CH₂Cl₂/MeOH (30:1) to afford tert-butyl 4-[7-({8-fluoro-2-methylimidazo[1,2-a]pyridin-6-yl}carbamoyl)-2-(pyrimidin-5-ylmethyl)indazol-4-yl]piperazine-1-carboxylate (50 mg, 44%) as an oil. LCMS (ES, m/z): 586 [M+H]⁺.

### Synthesis of Compound 318

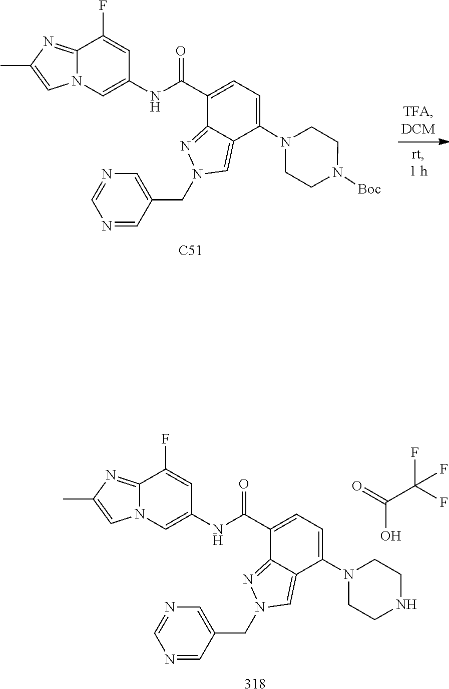

A mixture of tert-butyl 4-[7-({8-fluoro-2-methylimidazo[1,2-a]pyridin-6-yl}carbamoyl)-2-(pyrimidin-5-ylmethyl)indazol-4-yl]piperazine-1-carboxylate (80.0 mg, 0.137 mmol, 1.0 equiv), DCM (1 mL), and TFA (1 mL) was stirred for 1 h at room temperature. The resulting mixture was concentrated under reduced pressure to give a residue. The residue was purified by Prep-HPLC (Condition 3, Gradient 2) to afford N-{8-fluoro-2-methylimidazo[1,2-a]pyridin-6-yl}-4-(piperazin-1-yl)-2-(pyrimidin-5-ylmethyl)indazole-7-carboxamide trifluoroacetic acid salt (17.2 mg, 25%) as a solid. LCMS (ES, m/z): 486 [M+H]⁺. ¹H NMR (300 MHz, DMSO-d₆) δ 11.08 (s, 1H), 9.42 (d, J=1.6 Hz, 1H), 9.21 (s, 1H), 9.10 (d, J=20.0 Hz, 3H), 8.91 (d, J=6.1 Hz, 2H), 8.12-8.00 (m, 2H), 7.58 (d, J=12.0 Hz, 1H), 6.64 (d, J=8.1 Hz, 1H), 5.89 (s, 2H), 3.62-3.61 (m, 4H), 3.36-3.35 (m, 4H), 2.42 (s, 3H).

### Example 64: Synthesis of Compound 341

### Synthesis of Intermediate C52

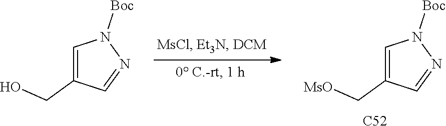 [SMILES: CC(C)(C)OC(=O)n1cc(CO)cn1 ; CC(C)(C)OC(=O)n1cc(COS(C)(=O)=O)cn1 ; "C52"]

Tert-butyl 4-(hydroxymethyl)pyrazole-1-carboxylate (400 mg, 2.018 mmol, 1.0 equiv), DCM (8 mL), triethylamine (408.40 mg, 4.036 mmol, 2 equiv), and methanesulfonyl chloride (300.48 mg, 2.623 mmol, 1.3 equiv) were combined at 0° C. The resulting mixture was stirred for 1 h at room temperature under nitrogen atmosphere, then quenched with water (50 mL) at 0° C. and extracted with DCM (2×50 mL). The organic layers were combined, dried over Na₂SO₄, and filtered. The filtrate was concentrated under reduced pressure to give a solid.

### Synthesis of Intermediate C53

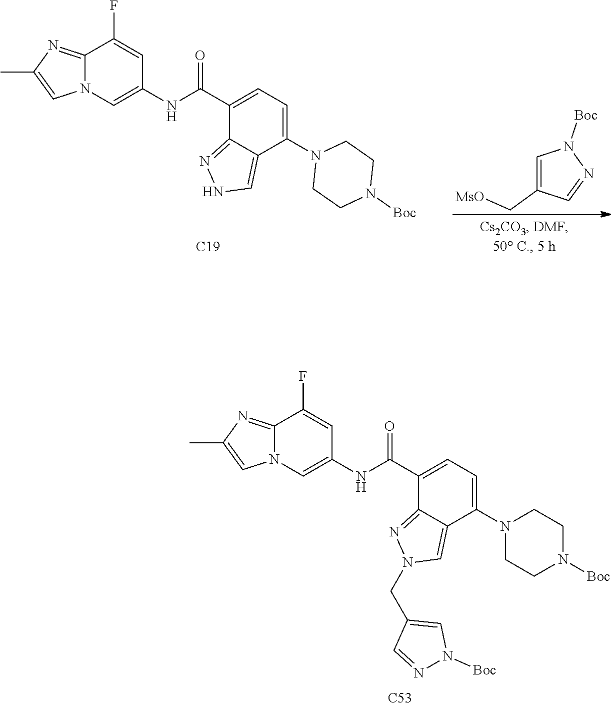 [SMILES: Cc1cn2cc(NC(=O)c3ccc(N4CCN(C(=O)OC(C)(C)C)CC4)c4c[nH]nc34)cc(F)c2n1 ; CC(C)(C)OC(=O)n1cc(COS(C)(=O)=O)cn1 ; "C19" ; Cc1cn2cc(NC(=O)c3ccc(N4CCN(C(=O)OC(C)(C)C)CC4)c4cn(Cc5cnn(C(=O)OC(C)(C)C)c5)nc34)cc(F)c2n1 ; "C53"]

A mixture of tert-butyl 4-[7-({8-fluoro-2-methylimidazo[1,2-a]pyridin-6-yl}carbamoyl)-2H-indazol-4-yl]piperazine-1-carboxylate (65.0 mg, 0.132 mmol, 1.0 equiv), caesio methaneperoxoate caesium (129.1 mg, 0.396 mmol, 3.0 equiv), dimethylformamide (2 mL) and tert-butyl 4-[(methanesulfonyloxy)methyl]pyrazole-1-carboxylate (54.5 mg, 0.198 mmol, 1.5 equiv) was stirred for 5 h at 50° C. The reaction mixture was cooled to room temperature, diluted with water (10 mL), and extracted with ethyl acetate (2×10 mL). The organic layers were combined, dried over Na₂SO₄, and filtered. The filtrate was concentrated under reduced pressure to give a residue. The residue was purified by silica gel column chromatography, eluted with CH₂Cl₂/MeOH (30:1) to afford tert-butyl 4-(2-{[1-(tert-butoxycarbonyl)pyrazol-4-yl]methyl}-7-({8-fluoro-2-methylimidazo[1,2-a]pyridin-6-yl}carbamoyl)indazol-4-yl)piperazine-1-carboxylate (45 mg, 47%) as a solid. LCMS (ES, m/z): 673 [M+H]⁺.

### Synthesis of Compound 341

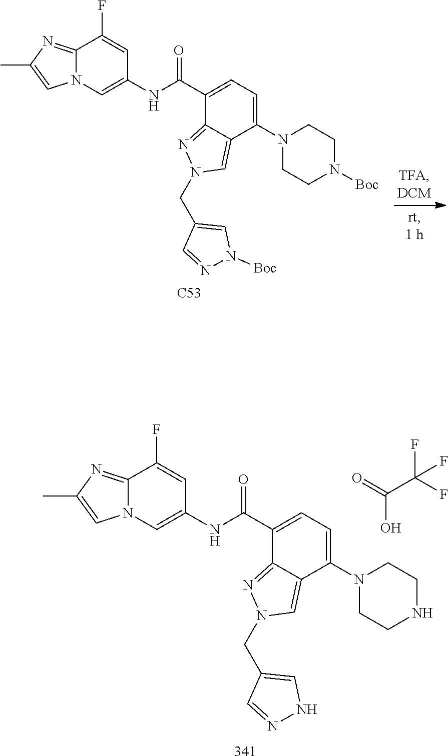

A mixture of tert-butyl 4-(2-{[1-(tert-butoxycarbonyl)pyrazol-4-yl]methyl}-7-({8-fluoro-2-methylimidazo[1,2-a]pyridin-6-yl}carbamoyl)indazol-4-yl)piperazine-1-carboxylate (40.0 mg, 0.059 mmol, 1.0 equiv), DCM (1 mL), and trifluoroacetaldehyde (1 mL, 10.202 mmol, 171.8 equiv) was stirred for 1 h at room temperature. The resulting mixture was concentrated under reduced pressure to give a residue. The residue was purified by Prep-HPLC (Condition 3, Gradient 2) to afford N-{8-fluoro-2-methylimidazo[1,2-a]pyridin-6-yl}-4-(piperazin-1-yl)-2-(1H-pyrazol-4-ylmethyl)indazole-7-carboxamide trifluoroacetic acid salt (18 mg, 64%) as a solid. LCMS (ES, m/z): 473 [M+H]⁺. ¹H NMR (400 MHz, DMSO-d₆) δ 11.25 (s, 1H), 9.40 (d, J=1.6 Hz, 1H), 8.96 (s, 1H), 8.92 (s, 2H), 8.09 (d, J=2.7 Hz, 1H), 8.03 (d, J=8.0 Hz, 1H), 7.85 (s, 2H), 7.61 (d, J=11.8 Hz, 1H), 6.62 (d, J=8.1 Hz, 1H), 5.69 (s, 2H), 3.60 (t, J=5.1 Hz, 4H), 3.36 (s, 4H), 2.43 (s, 3H).

### Example 65: Synthesis of Compound 326

### Synthesis of Intermediate C54

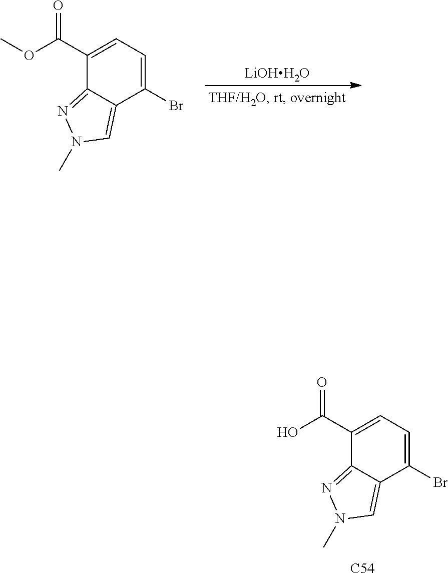

To a stirred mixture of methyl 4-bromo-2-methylindazole-7-carboxylate (1.6 g, 5.946 mmol, 1 equiv) in THE (1.5 mL) and water (0.5 mL) was added lithiumol hydrate (0.50 g, 11.892 mmol, 2 equiv) at room temperature. The resulting mixture was stirred overnight at room temperature. The resulting solution was concentrated under reduced pressure and acidified to pH 6 with citric acid to afford a precipitate. The precipitated solid was collected by filtration and dried under infrared light to afford 4-bromo-2-methylindazole-7-carboxylic acid (1.3 g, 86%) as a solid. LCMS (ES, m/z): 255 [M+H]⁺.

### Synthesis of Intermediate C55

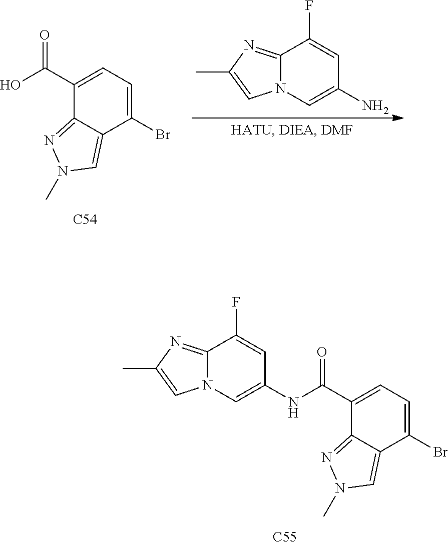 [SMILES: Cn1cc2c(Br)ccc(C(=O)O)c2n1 ; Cc1cn2cc(N)cc(F)c2n1 ; "C54" ; Cc1cn2cc(NC(=O)c3ccc(Br)c4cn(C)nc34)cc(F)c2n1 ; "C55"]

To a stirred mixture of 4-bromo-2-methylindazole-7-carboxylic acid (500 mg, 1.960 mmol, 1 equiv) and DIEA (760.06 mg, 5.880 mmol, 3 equiv) in DMF (10 mL) was added HATU (968.96 mg, 2.548 mmol, 1.3 equiv) and 8-fluoro-2-methylimidazo[1,2-a]pyridin-6-amine (420.91 mg, 2.548 mmol, 1.3 equiv) at room temperature. The resulting mixture was stirred overnight at room temperature and filtrated. The filter cake was dried under infrared light to afford 4-bromo-N-{8-fluoro-2-methylimidazo[1,2-a]pyridin-6-yl}-2-methylindazole-7-carboxamide (600 mg, 76%) as a solid. LCMS (ES, m/z): 402 [M+H]⁺.

### Synthesis of Intermediate C56

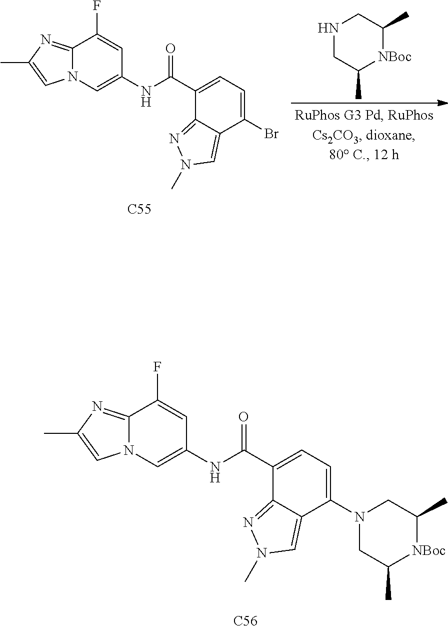

To a stirred mixture of 4-bromo-N-{8-fluoro-2-methylimidazo[1,2-a]pyridin-6-yl}-2-methylindazole-7-carboxamide (70 mg, 0.174 mmol, 1 equiv) and tert-butyl (2R,6S)-2,6-dimethylpiperazine-1-carboxylate (44.76 mg, 0.209 mmol, 1.2 equiv) in 1,4-dioxane (1.4 mL) was added Cs₂CO₃ (170.11 mg, 0.522 mmol, 3.0 equiv), RuPhos (8.12 mg, 0.017 mmol, 0.1 equiv), and RuPhos Palladacycle Gen.3 (14.56 mg, 0.017 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl (2R,6S)-4-[7-({8-fluoro-2-methylimidazo[1,2-a]pyridin-6-yl}carbamoyl)-2-methylindazol-4-yl]-2,6-dimethylpiperazine-1-carboxylate (70 mg, 56%) as a solid. LCMS (ES, m/z): 536 [M+H]⁺.

### Synthesis of Compound 326

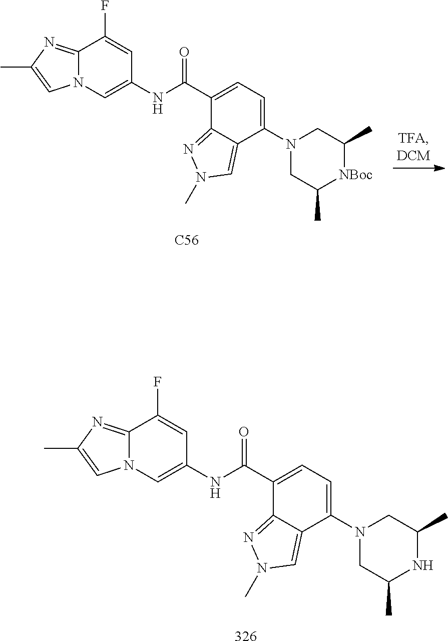

To a stirred solution of tert-butyl(2R,6S)-4-[7-({8-fluoro-2-methylimidazo[1,2-a]pyridin-6-yl}carbamoyl)-2-methylindazol-4-yl]-2,6-dimethylpiperazine-1-carboxylate (70 mg, 0.131 mmol, 1 equiv) in DCM (1 mL) was added TFA (1 mL, 13.463 mmol, 103.02 equiv) at room temperature. The resulting mixture was stirred for 1 h at room temperature, then concentrated under reduced pressure to give a residue. The residue was purified by Prep-HPLC (Condition 5, Gradient 1) to afford 4-[(3R,5S)-3,5-dimethylpiperazin-1-yl]-N-{8-fluoro-2-methylimidazo[1,2-a]pyridin-6-yl}-2-methylindazole-7-carboxamide (11.1 mg, 20%) as a solid. LCMS (ES, m/z): 436 [M+H]⁺. ¹H NMR (400 MHz, DMSO-d6) δ 11.08 (s, 1H), 9.21 (d, J=1.6 Hz, 1H), 8.80 (s, 1H), 7.97 (d, J=8.1 Hz, 1H), 7.90 (d, J=3.1 Hz, 1H), 7.35 (d, J=12.6 Hz, 1H), 6.48 (d, J=8.2 Hz, 1H), 4.30 (s, 3H), 3.79 (d, J=11.7 Hz, 2H), 2.95 (s, 3H), 2.49-2.44 (m, 2H), 2.35 (s, 3H), 1.07 (d, J=6.2 Hz, 6H).

### Example 66: Synthesis of Compound 345

### Synthesis of Intermediate C57

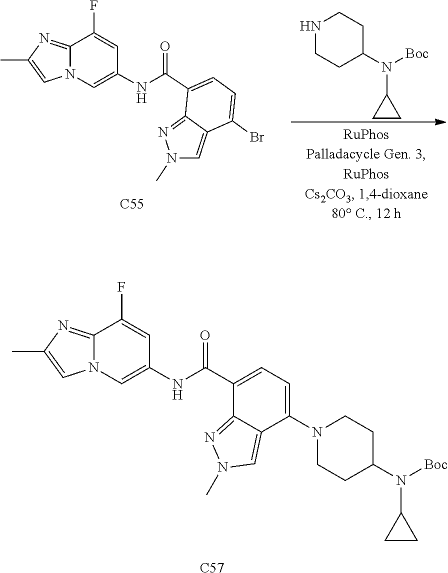 [SMILES: Cc1cn2cc(NC(=O)c3ccc(Br)c4cn(C)nc34)cc(F)c2n1 ; CC(C)(C)OC(=O)N(C1CCNCC1)C1CC1 ; "C55" ; Cc1cn2cc(NC(=O)c3ccc(N4CCC(N(C(=O)OC(C)(C)C)C5CC5)CC4)c4cn(C)nc34)cc(F)c2n1 ; "C57"]

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-cyclopropyl-N-(piperidin-4-yl)carbamate (43.02 mg, 0.179 mmol, 1.2 equiv) in 1,4-dioxane (1.2 mL) was added Cs₂CO₃ (145.81 mg, 0.447 mmol, 3 equiv), RuPhos (6.96 mg, 0.015 mmol, 0.1 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl N-cyclopropyl-N-{1-[7-({8-fluoro-2-methylimidazo[1,2-a]pyridin-6-yl}carbamoyl)-2-methylindazol-4-yl]piperidin-4-yl}carbamate (50 mg, 42%) as an oil. LCMS (ES, m/z): 562 [M+H]+

### Synthesis of Compound 345

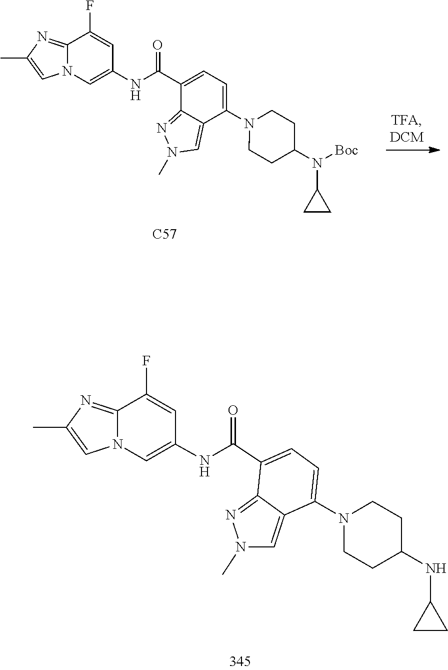 [SMILES: Cc1cn2cc(NC(=O)c3ccc(N4CCC(N(C(=O)OC(C)(C)C)C5CC5)CC4)c4cn(C)nc34)cc(F)c2n1 ; "C57" ; Cc1cn2cc(NC(=O)c3ccc(N4CCC(NC5CC5)CC4)c4cn(C)nc34)cc(F)c2n1]

To a stirred solution of tert-butyl N-cyclopropyl-N-{1-[7-({8-fluoro-2-methylimidazo[1,2-a]pyridin-6-yl}carbamoyl)-2-methylindazol-4-yl]piperidin-4-yl}carbamate (50 mg, 0.089 mmol, 1 equiv) in DCM (1 mL) was added TFA (1 mL, 13.463 mmol, 151.23 equiv) at room temperature. The resulting mixture was stirred for 1 h at room temperature, then concentrated under reduced pressure to give a residue. The residue was purified by Prep-HPLC (Condition 5, Gradient 2) to afford 4-[4-(cyclopropylamino)piperidin-1-yl]-N-{8-fluoro-2-methylimidazo[1,2-a]pyridin-6-yl}-2-methylindazole-7-carboxamide (3.6 mg, 9%) as solid. LCMS (ES, m/z): 462 [M+H]⁺. ¹H NMR (400 MHz, DMSO-d₆) δ 11.07 (s, 1H), 9.21 (d, J=1.6 Hz, 1H), 8.78 (s, 1H), 7.97 (d, J=8.1 Hz, 1H), 7.90 (d, J=3.0 Hz, 1H), 7.34 (dd, J=12.4, 1.6 Hz, 1H), 6.49 (d, J=8.2 Hz, 1H), 4.30 (s, 3H), 3.89 (d, J=12.6 Hz, 2H), 3.11-3.02 (m, 2H), 2.79 (s, 1H), 2.35 (s, 3H), 2.13 (dt, J=6.6, 3.1 Hz, 1H), 2.01 (d, J=12.6 Hz, 2H), 1.48 (q, J=10.7, 9.9 Hz, 2H), 0.40 (dt, J=6.3, 3.0 Hz, 2H), 0.24 (p, J=3.9 Hz, 2H).

### Example 67: Synthesis of Compound 335

### Synthesis of Intermediate C58

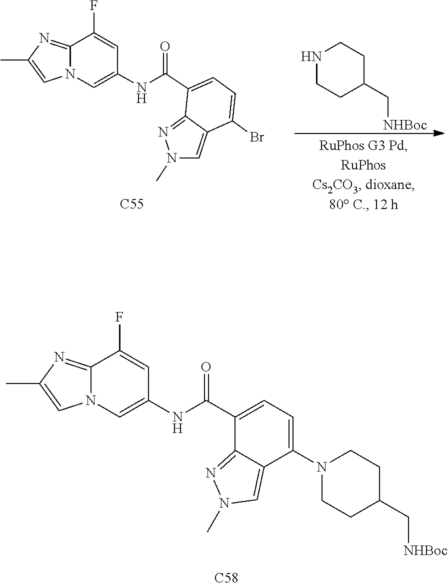 [SMILES: Cc1cn2cc(NC(=O)c3ccc(Br)c4cn(C)nc34)cc(F)c2n1 ; CC(C)(C)OC(=O)NCC1CCNCC1 ; "C55" ; Cc1cn2cc(NC(=O)c3ccc(N4CCC(CNC(=O)OC(C)(C)C)CC4)c4cn(C)nc34)cc(F)c2n1 ; "C58"]

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-(piperidin-4-ylmethyl)carbamate (38.36 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl N-({1-[7-({8-fluoro-2-methylimidazo[1,2-a]pyridin-6-yl}carbamoyl)-2-methylindazol-4-yl]piperidin-4-yl}methyl)carbamate (60 mg, 75%) as a solid. LCMS (ES, m/z): 536 [M+H]⁺.

### Synthesis of Compound 335

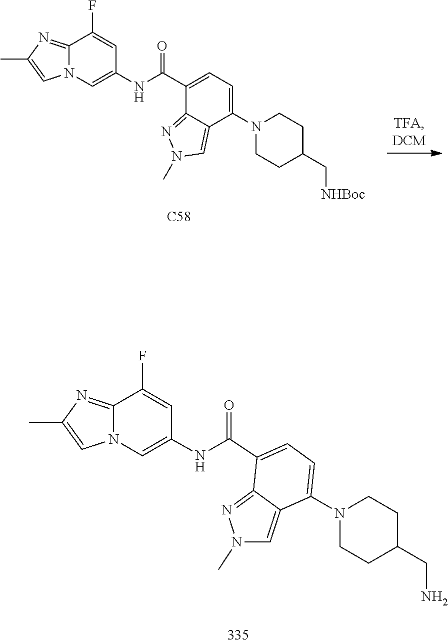 [SMILES: Cc1cn2cc(NC(=O)c3ccc(N4CCC(CNC(=O)OC(C)(C)C)CC4)c4cn(C)nc34)cc(F)c2n1 ; "C58" ; Cc1cn2cc(NC(=O)c3ccc(N4CCC(CN)CC4)c4cn(C)nc34)cc(F)c2n1]

To a stirred solution of tert-butyl N-({1-[7-({8-fluoro-2-methylimidazo[1,2-a]pyridin-6-yl}carbamoyl)-2-methylindazol-4-yl]piperidin-4-yl}methyl)carbamate (60 mg, 0.112 mmol, 1 equiv) in DCM (1 mL) was added TFA (1 mL, 13.463 mmol, 120.19 equiv) at room temperature. The resulting mixture was stirred for 1 h at room temperature, then concentrated under reduced pressure to give a residue. The residue was purified by Prep-HPLC (Condition 5, Gradient 1) to afford 4-[4-(aminomethyl)piperidin-1-yl]-N-{8-fluoro-2-methylimidazo[1,2-a]pyridin-6-yl}-2-methylindazole-7-carboxamide (30.5 mg, 63%) as a solid. LCMS (ES, m/z): 436 [M+H]⁺. ¹H NMR (300 MHz, DMSO-d₆) δ 11.07 (s, 1H), 9.21 (d, J=1.6 Hz, 1H), 8.76 (d, J=3.0 Hz, 1H), 7.97 (d, J=8.1 Hz, 1H), 7.90 (d, J=3.1 Hz, 1H), 7.34 (dd, J=12.4, 1.7 Hz, 1H), 6.49 (d, J=8.2 Hz, 1H), 4.30 (s, 3H), 3.97 (d, J=11.7 Hz, 2H), 2.96 (s, 3H), 2.38-2.32 (m, 3H), 1.83 (t, J=15.5 Hz, 2H), 1.60 (d, J=44.4 Hz, 1H), 1.41-1.27 (m, 2H).

### Example 68: Synthesis of Compound 327

### Synthesis of Intermediate C59

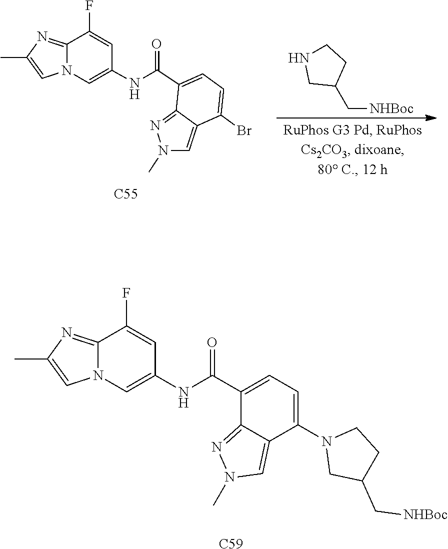 [SMILES: Cc1cn2cc(NC(=O)c3ccc(Br)c4cn(C)nc34)cc(F)c2n1 ; CC(C)(C)OC(=O)NCC1CCNC1 ; "C55" ; Cc1cn2cc(NC(=O)c3ccc(N4CCC(CNC(=O)OC(C)(C)C)C4)c4cn(C)nc34)cc(F)c2n1 ; "C59"]

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-(pyrrolidin-3-ylmethyl)carbamate (35.85 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl N-({1-[7-({8-fluoro-2-methylimidazo[1,2-a]pyridin-6-yl}carbamoyl)-2-methylindazol-4-yl]pyrrolidin-3-yl}methyl)carbamate (70 mg, 90%) as a solid. LCMS (ES, m/z): 522 [M+H]⁺.

### Synthesis of Compound 327

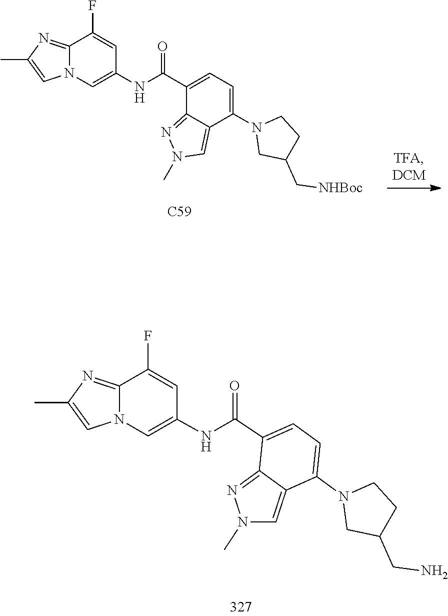 [SMILES: Cc1cn2cc(NC(=O)c3ccc(N4CCC(CNC(=O)OC(C)(C)C)C4)c4cn(C)nc34)cc(F)c2n1 ; "C59" ; Cc1cn2cc(NC(=O)c3ccc(N4CCC(CN)C4)c4cn(C)nc34)cc(F)c2n1]

To a stirred solution of tert-butyl N-({1-[7-({8-fluoro-2-methylimidazo[1,2-a]pyridin-6-yl}carbamoyl)-2-methylindazol-4-yl]pyrrolidin-3-yl}methyl)carbamate (70 mg, 0.134 mmol, 1 equiv) in DCM (1 mL) was added TFA (1 mL, 100.32 equiv) at room temperature. The resulting mixture was stirred for 1 h at room temperature. The resulting mixture was concentrated under reduced pressure to give a residue. The residue was purified by Prep-HPLC (Condition 5, Gradient 3) to afford 4-[3-(aminomethyl)pyrrolidin-1-yl]-N-{8-fluoro-2-methylimidazo[1,2-a]pyridin-6-yl}-2-methylindazole-7-carboxamide (16.2 mg, 29%) as a solid. LCMS (ES, m/z): 422 [M+H]⁺. ¹H NMR (400 MHz, DMSO-d6) δ 11.01 (s, 1H), 9.19 (d, J=1.6 Hz, 1H), 8.82 (d, J=11.3 Hz, 1H), 7.96-7.86 (m, 2H), 7.30 (dd, J=12.5, 1.7 Hz, 1H), 6.02 (dd, J=8.4, 2.2 Hz, 1H), 4.27 (s, 3H), 3.82-3.57 (m, 3H), 3.19-3.04 (m, 1H), 2.69 (d, J=6.6 Hz, 1H), 2.39 (t, J=7.4 Hz, 1H), 2.35 (s, 3H), 2.14 (s, 1H), 1.81 (q, J=11.3, 9.3 Hz, 1H).

### Example 69: Synthesis of Compound 328

### Synthesis of Compound 328

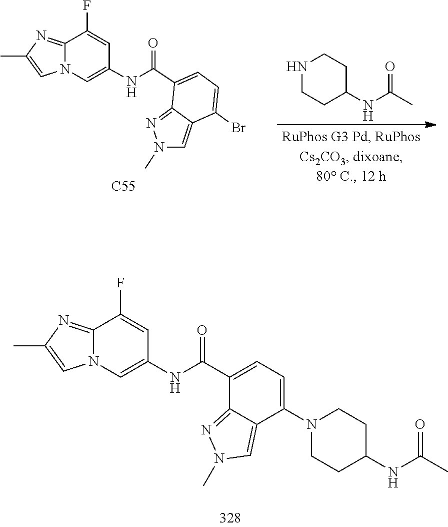

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and N-(piperidin-4-yl)acetamide (25.45 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1), followed by Prep-HPLC (Condition 5, Gradient 4) to afford 4-(4-acetamidopiperidin-1-yl)-N-{8-fluoro-2-methylimidazo[1,2-a]pyridin-6-yl}-2-methylindazole-7-carboxamide (15 mg, 21.69%) as a solid. LCMS (ES, m/z): 464 [M+H]⁺. ¹H NMR (400 MHz, DMSO-d₆) δ 11.07 (s, 1H), 9.21 (d, J=1.7 Hz, 1H), 8.79 (s, 1H), 7.98 (d, J=8.1 Hz, 1H), 7.90 (t, J=4.9 Hz, 2H), 7.34 (dd, J=12.4, 1.7 Hz, 1H), 6.51 (d, J=8.2 Hz, 1H), 4.30 (s, 3H), 3.94-3.78 (m, 3H), 3.12 (t, J=12.0 Hz, 2H), 2.35 (s, 3H), 1.90 (d, J=12.5 Hz, 2H), 1.83 (s, 3H), 1.57 (q, J=10.6 Hz, 2H).

### Example 70: Synthesis of Compound 336

### Synthesis of Intermediate C60

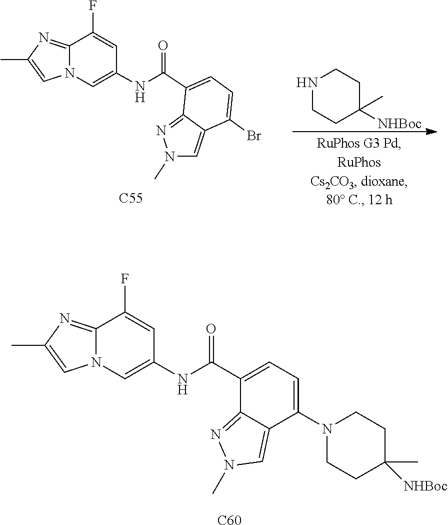

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-(4-methylpiperidin-4-yl)carbamate (38.36 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl N-{1-[7-({8-fluoro-2-methylimidazo[1,2-a]pyridin-6-yl}carbamoyl)-2-methylindazol-4-yl]-4-methylpiperidin-4-yl}carbamate (65 mg, 81%) as a solid. LCMS (ES, m/z): 536 [M+H]⁺.

### Synthesis of Compound 336

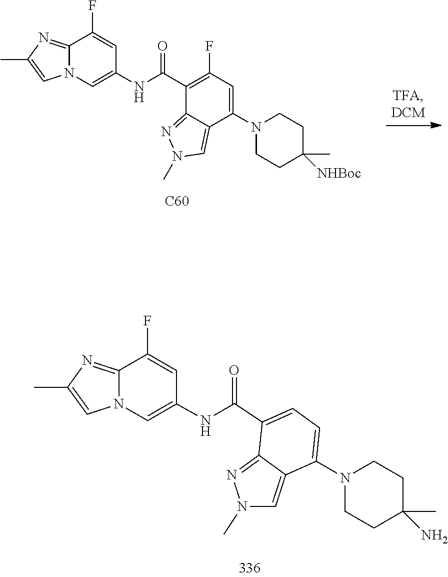

To a stirred solution of tert-butyl N-{1-[7-({8-fluoro-2-methylimidazo[1,2-a]pyridin-6-yl}carbamoyl)-2-methylindazol-4-yl]-4-methylpiperidin-4-yl}carbamate (65 mg, 0.121 mmol, 1 equiv) in DCM (1 mL) was added TFA (1 mL, 13.463 mmol, 110.94 equiv) at room temperature. The resulting mixture was stirred for 1 h at room temperature, then concentrated under reduced pressure to give a residue. The residue was purified by Prep-HPLC (Condition 5, Gradient 1) to afford 4-(4-amino-4-methylpiperidin-1-yl)-N-{8-fluoro-2-methylimidazo[1,2-a]pyridin-6-yl}-2-methylindazole-7-carboxamide (15.5 mg, 29%) as a solid. LCMS (ES, m/z): 436 [M+H]⁺. ¹H NMR (400 MHz, DMSO-d₆) δ 11.07 (s, 1H), 9.21 (d, J=1.7 Hz, 1H), 8.78 (s, 1H), 7.97 (d, J=8.2 Hz, 1H), 7.90 (d, J=3.0 Hz, 1H), 7.34 (dd, J=12.3, 1.7 Hz, 1H), 6.50 (d, J=8.2 Hz, 1H), 4.29 (s, 3H), 3.53 (s, 4H), 2.35 (d, J=0.9 Hz, 3H), 1.68-1.55 (m, 4H), 1.17 (s, 3H).

### Example 71: Synthesis of Compound 346

### Synthesis of Intermediate C61

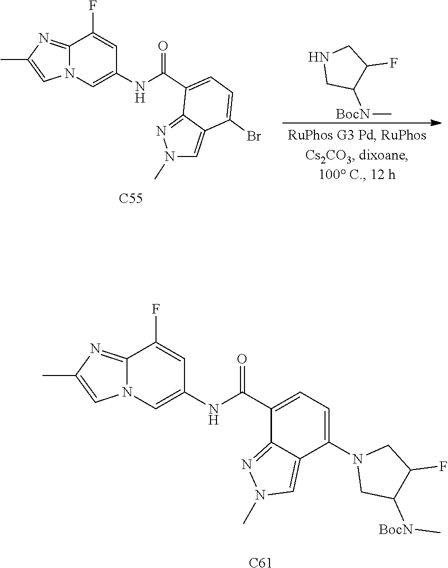

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-(4-fluoropyrrolidin-3-yl)-N-methylcarbamate (39.07 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 100° C. under nitrogen atmosphere, then concentrated under reduced pressure to give a residue. The residue was purified by silica gel column chromatography, eluted with CH₂Cl₂/MeOH (20:1) to afford tert-butyl N-{4-fluoro-1-[7-({8-fluoro-2-methylimidazo[1,2-a]pyridin-6-yl}carbamoyl)-2-methylindazol-4-yl]pyrrolidin-3-yl}-N-methylcarbamate (70 mg, 87%) as a solid. LCMS (ES, m/z): 540 [M+H]⁺.

### Synthesis of Compound 346

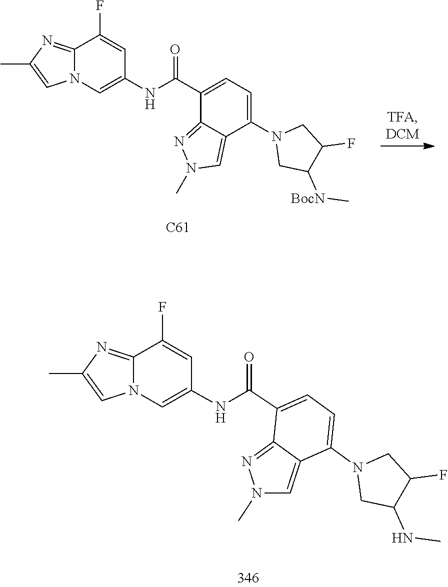 [SMILES: Cc1cn2cc(NC(=O)c3ccc(N4CC(F)C(N(C)C(=O)OC(C)(C)C)C4)c4cn(C)nc34)cc(F)c2n1 ; "C61" ; CNC1CN(c2ccc(C(=O)Nc3cc(F)c4nc(C)cn4c3)c3nn(C)cc23)CC1F]

To a stirred solution of tert-butyl N-{4-fluoro-1-[7-({8-fluoro-2-methylimidazo[1,2-a]pyridin-6-yl}carbamoyl)-2-methylindazol-4-yl]pyrrolidin-3-yl}-N-methylcarbamate (70 mg, 0.130 mmol, 1 equiv) in DCM (1 mL) was added TFA (1 mL, 13.463 mmol, 103.78 equiv) at room temperature. The resulting mixture was stirred for 1 h at room temperature, then concentrated under reduced pressure to give a residue. The residue was purified by Prep-HPLC (Condition 5, Gradient 4) to afford N-{8-fluoro-2-methylimidazo[1,2-a]pyridin-6-yl}-4-[3-fluoro-4-(methylamino)pyrrolidin-1-yl]-2-methylindazole-7-carboxamide (3.4 mg, 6%) as a solid. LCMS (ES, m/z): 440 [M+H]⁺. ¹H NMR (400 MHz, DMSO-d₆) δ 11.02 (s, 1H), 9.20 (d, J=1.7 Hz, 1H), 8.86 (s, 1H), 7.95 (d, J=8.2 Hz, 1H), 7.92-7.86 (m, 1H), 7.32 (dd, J=12.4, 1.7 Hz, 1H), 6.06 (d, J=8.4 Hz, 1H), 5.40 (d, J=54.5 Hz, 1H), 4.28 (s, 3H), 3.94 (td, J=25.2, 21.2, 9.8 Hz, 3H), 2.44 (s, 3H), 2.37-2.33 (m, 3H).

### Example 72: Synthesis of Compound 329

### Synthesis of Compound 329

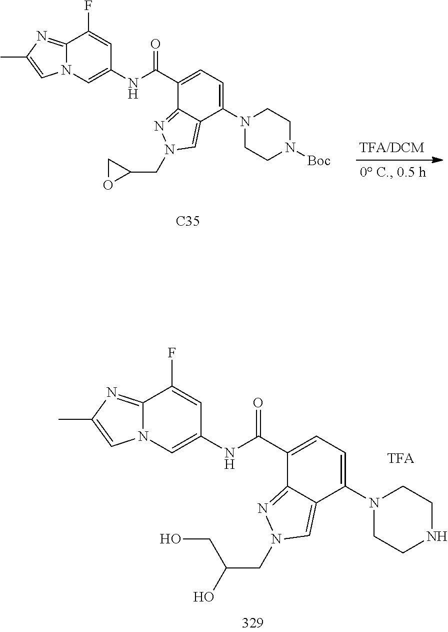 [SMILES: Cc1cn2cc(NC(=O)c3ccc(N4CCN(C(=O)OC(C)(C)C)CC4)c4cn(CC5CO5)nc34)cc(F)c2n1 ; "C35" ; Cc1cn2cc(NC(=O)c3ccc(N4CCNCC4)c4cn(CC(O)CO)nc34)cc(F)c2n1]

A solution of tert-butyl 4-[7-({8-fluoro-2-methylimidazo[1,2-a]pyridin-6-yl}carbamoyl)-2-(oxiran-2-ylmethyl)indazol-4-yl]piperazine-1-carboxylate (38.0 mg, 0.07 mmol, 1.0 equiv) in DCM (0.4 mL) was treated with TFA (0.4 mL) at room temperature. The resulting mixture was stirred for 30 min at 0° C., then concentrated under reduced pressure to give a residue. The residue was purified by Prep-HPLC (Condition 3, Gradient 12) to afford 2-(2,3-dihydroxypropyl)-N-{8-fluoro-2-methylimidazo[1,2-a]pyridin-6-yl}-4-(piperazin-1-yl)indazole-7-carboxamide trifluoroacetic acid salt (10.8 mg, 28%) as a solid. LCMS (ES, m/z): 468 [M+H]⁺. ¹H NMR (300 MHz, DMSO-d₆) δ 11.32 (s, 1H), 9.44 (s, 1H), 9.07 (s, 2H), 8.88 (s, 1H), 8.11 (d, J=2.7 Hz, 1H), 8.03 (d, J=7.9 Hz, 1H), 7.66 (d, J=11.7 Hz, 1H), 6.62 (d, J=8.1 Hz, 1H), 4.69 (dd, J=13.5, 3.7 Hz, 1H), 4.48 (dd, J=13.5, 7.7 Hz, 1H), 4.14 (d, J=6.4 Hz, 1H), 3.62 (d, J=5.5 Hz, 4H), 3.48 (td, J=11.0, 10.3, 5.5 Hz, 2H), 3.37 (s, 4H), 2.43 (s, 3H).

### Example 73: Synthesis of Compound 356

### Synthesis of Intermediate C62

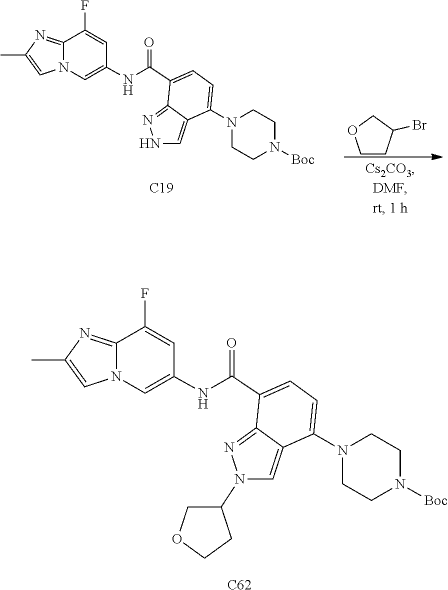 [SMILES: Cc1cn2cc(NC(=O)c3ccc(N4CCN(C(=O)OC(C)(C)C)CC4)c4c[nH]nc34)cc(F)c2n1 ; BrC1CCOC1 ; "C19" ; Cc1cn2cc(NC(=O)c3ccc(N4CCN(C(=O)OC(C)(C)C)CC4)c4cn(C5CCOC5)nc34)cc(F)c2n1 ; "C62"]

A mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.2 mmol, 1.0 equiv), 3-bromooxolane (45.8 mg, 0.3 mmol, 1.5 equiv), and Cs₂CO₃ (198.0 mg, 0.6 mmol, 3.0 equiv) in DMF (1 mL) was stirred for 1 h at room temperature under nitrogen atmosphere. The reaction mixture was diluted with water (3 mL) and extracted with ethyl acetate (2×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with PE/EA (1:4) to afford tert-butyl-4-[7-({8-fluoro-2-methylimidazo[1,2-a]pyridin-6-yl}carbamoyl)-2-(oxolan-3-yl)indazol-4-yl]piperazine-1-carboxylate (52.0 mg, 46%) as a solid. LCMS (ES, m/z): 564 [M+H]⁺.

### Synthesis of Compound 356

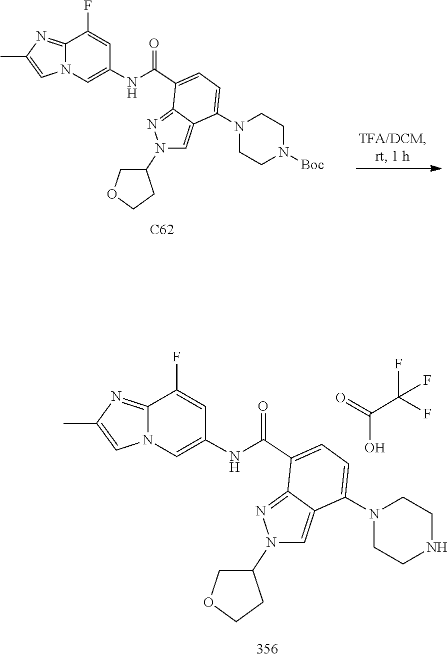

A solution of tert-butyl 4-[7-({8-fluoro-2-methylimidazo[1,2-a]pyridin-6-yl}carbamoyl)-2-(oxolan-3-yl)indazol-4-yl]piperazine-1-carboxylate (52.0 mg, 0.09 mmol, 1.0 equiv) in DCM (0.5 mL) was treated with DCM (0.5 mL) at room temperature. The resulting mixture was stirred for 30 min at room temperature, then concentrated under reduced pressure to give a residue. The residue was purified by Prep-HPLC (Condition 3, Gradient 13) to afford N-{8-fluoro-2-methylimidazo[1,2-a]pyridin-6-yl}-2-(oxolan-3-yl)-4-(piperazin-1-yl)indazole-7-carboxamide trifluoroacetic acid salt (17.1 mg, 33%) as a solid. LCMS (ES, m/z): 464 [M+H]⁺. ¹H NMR (400 MHz, DMSO-d₆) δ 11.32 (s, 1H), 9.47 (s, 1H), 9.07 (s, 2H), 8.94 (s, 1H), 8.17 (s, 1H), 8.04 (d, J=7.9 Hz, 1H), 7.63 (d, J=11.7 Hz, 1H), 6.63 (d, J=8.1 Hz, 1H), 5.52 (ddt, J=9.3, 6.8, 3.4 Hz, 2H), 4.33-4.09 (m, 3H), 3.98 (td, J=8.2, 5.3 Hz, 1H), 3.77-3.57 (m, 4H), 3.37 (s, 4H), 2.63 (dq, J=15.4, 7.7 Hz, 1H), 2.45 (s, 3H).

### Example 74: Synthesis of Compounds 350 and 351 Synthesis of Compounds 350 and 351

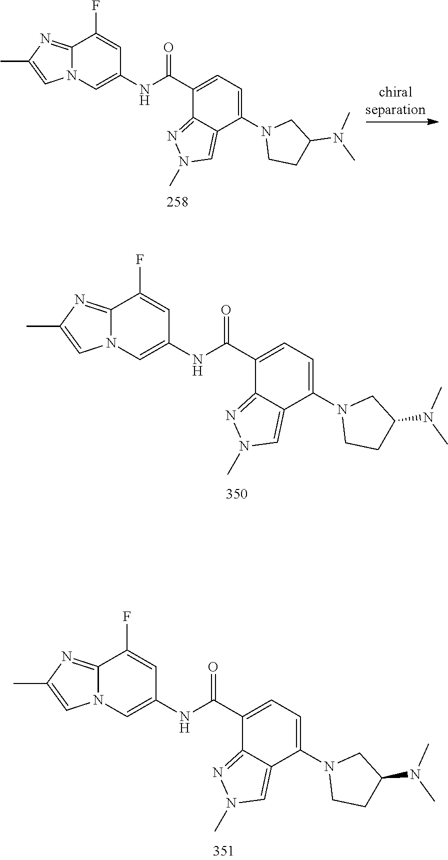

4-[3-(dimethylamino)pyrrolidin-1-yl]-N-{8-fluoro-2-methylimidazo[1,2-a]pyridin-6-yl}-2-methylindazole-7-carboxamide (20 mg) was purified by PREP-CHIRAL-HPLC (Condition 1, Gradient 1) to afford Compound 350 (First peak: RT (min): 5.9) (5 mg) and Compound 351 (Second peak: RT (min): 6.4) (5 mg) as solids. Compound 350: LCMS: (ES, m/z): 436 [M+H]⁺. ¹H NMR: (400 MHz, DMSO-d₆) δ 11.02 (d, J=2.4 Hz, 1H), 9.20 (t, J=2.1 Hz, 1H), 8.87 (d, J=2.4 Hz, 1H), 7.97-7.86 (m, 2H), 7.36-7.28 (m, 1H), 6.05 (dd, J=8.3, 2.4 Hz, 1H), 4.28 (d, J=2.4 Hz, 3H), 3.85 (s, 1H), 3.77 (s, 1H), 3.65 (d, J=9.3 Hz, 1H), 3.46 (s, 2H), 2.89 (s, 1H), 2.35 (d, J=2.4 Hz, 6H), 2.29 (s, 3H), 1.92 (s, 1H). Compound 351: LCMS: (ES, m/z): 436 [M+H]⁺. ¹H NMR: (400 MHz, DMSO-d₆) δ 11.02 (s, 1H), 9.20 (d, J=1.7 Hz, 1H), 8.87 (s, 1H), 7.96-7.86 (m, 2H), 7.31 (dd, J=12.4, 1.7 Hz, 1H), 6.05 (d, J=8.4 Hz, 1H), 4.28 (s, 3H), 3.85 (t, J=8.6 Hz, 1H), 3.77 (t, J=9.4 Hz, 1H), 3.64 (q, J=8.9 Hz, 1H), 3.48 (d, J=9.0 Hz, 2H), 2.91 (s, 1H), 2.37-2.33 (m, 3H), 2.29 (s, 6H), 1.92 (t, J=10.3 Hz, 1H).

### Example 75: Synthesis of Compound 288

### Synthesis of Intermediate C63

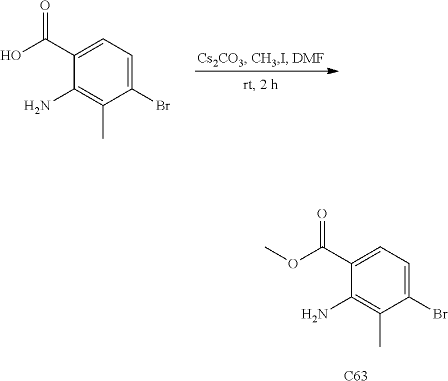

To a stirred mixture of 2-amino-4-bromo-3-methylbenzoic acid (10 g, 43.467 mmol, 1.0 equiv) and Cs₂CO₃ (21.2 g, 65.201 mmol, 1.5 equiv) in DMF (100 mL) was added CH₃I (7.4 g, 52.160 mmol, 1.2 equiv) in portions at 0° C. under N₂ atmosphere. The resulting mixture was stirred for 2 h at room temperature under N₂ atmosphere. The resulting mixture was diluted with water (300 mL) and extracted with ethyl acetate (2×300 mL). The organic layers were combined, washed with water (3×400 mL), dried over anhydrous Na₂SO₄, and filtered. After filtration, the filtrate was concentrated under reduced pressure to afford methyl 2-amino-4-bromo-3-methylbenzoate (10.4 g, 98%) as a solid. LCMS (ES, m/z): 244 [M+H]⁺.

### Synthesis of Intermediate C64

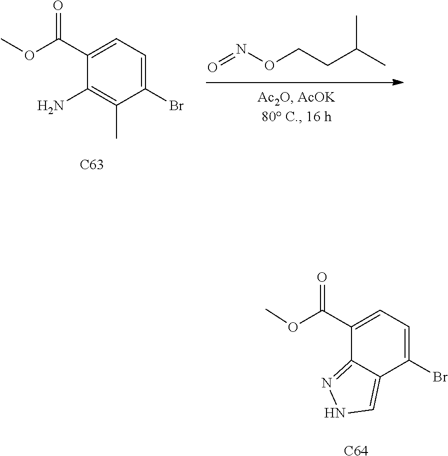

A mixture of methyl 2-amino-4-bromo-3-methylbenzoate (10 g, 40.969 mmol, 1.00 equiv) and Ac₂O (5.02 g, 49.163 mmol, 1.2 equiv) in CHCl₃ (200 mL) was stirred for 1 h at room temperature under N₂ atmosphere. To the reaction mixture was added AcOK (1.21 g, 12.291 mmol, 0.3 equiv) and aspiral (1.056 mg, 90.132 mmol, 2.2 equiv). The resulting mixture was stirred for 16 h at 80° C. under N₂ atmosphere. The precipitate formed that was collected by filtration and washed with isopropanol (2×50 mL). The resulting solid was dried to afford methyl 4-bromo-2H-indazole-7-carboxylate (10.1 g, 97%) as a solid. LCMS (ES, m/z): 255 [M+H]⁺.

### Synthesis of Intermediate C65

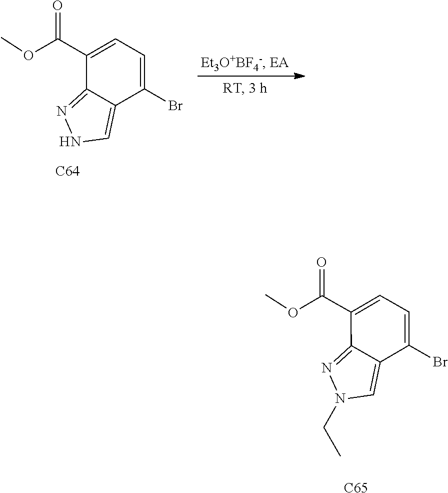

To a stirred solution of methyl 4-bromo-2H-indazole-7-carboxylate (1 g, 3.920 mmol, 1.00 equiv) in ethyl acetate (7.5 mL) was added Et₃O⁺BF₄⁻ (3724.20 mg, 19.600 mmol, 5.0 equiv) in portions at room temperature. The resulting mixture was stirred for 3 h at room temperature. The resulting mixture was diluted with water (10 mL) and extracted with ethyl acetate (3×20 mL). The organic layers were combined, washed with water (2×20 mL), dried over anhydrous Na₂SO₄, and filtered. After filtration, the filtrate was concentrated under reduced pressure to residue. The residue was purified by silica gel column chromatography, eluted with PE/EA (1:1) to afford methyl 4-bromo-2-ethylindazole-7-carboxylate (610 mg, 55%) as a solid. LCMS (ES, m/z): 283 [M+H]⁺.

### Synthesis of Intermediate C66

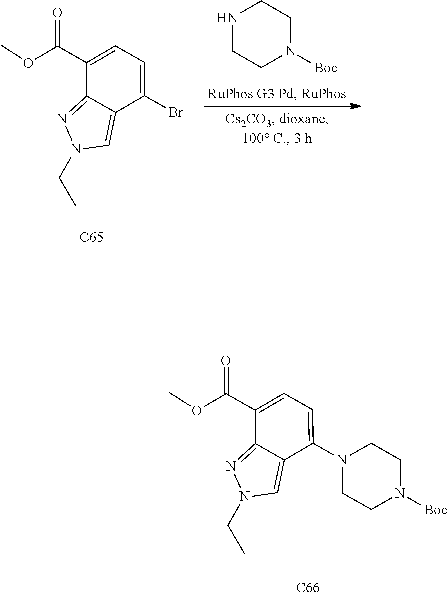

To a stirred mixture of methyl 4-bromo-2-ethylindazole-7-carboxylate (250 mg, 0.883 mmol, 1.0 equiv) and tert-butyl piperazine-1-carboxylate (328.9 mg, 1.766 mmol, 2.0 equiv) in dioxane (5 mL) was added Cs₂CO₃ (575.4 mg, 1.766 mmol, 2.0 equiv), RuPhos Palladacycle Gen.3 (73.9 mg, 0.088 mmol, 0.1 equiv), and RuPhos (82.4 mg, 0.177 mmol, 0.2 equiv) at room temperature under N₂ atmosphere. The resulting mixture was stirred for 3 h at 100° C. under nitrogen atmosphere, then concentrated under reduced pressure and purified by silica gel column chromatography, eluted with ethyl acetate to afford methyl 4-[4-(tert-butoxycarbonyl)piperazin-1-yl]-2-ethylindazole-7-carboxylate (240 mg, 70%) as a solid. LCMS (ES, m/z): 389 [M+H]⁺.

### Synthesis of Intermediate C67

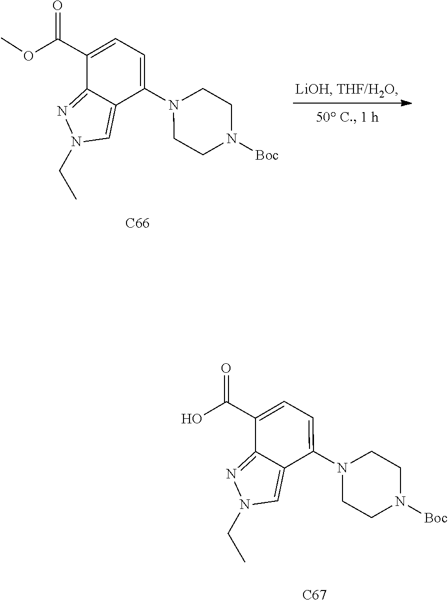

To a stirred mixture of methyl 4-[4-(tert-butoxycarbonyl)piperazin-1-yl]-2-ethylindazole-7-carboxylate (210 mg, 0.541 mmol, 1.00 equiv) in THE (1.1 mL) and water (0.55 mL) was added LiOH (51.78 mg, 2.164 mmol, 4.0 equiv) at room temperature. The resulting mixture was stirred for 1 h at 50° C. The resulting mixture was concentrated under reduced pressure, diluted with water (2 mL), acidified to pH 7 with HCl (1 N), and extracted with ethyl acetate (3×3 mL). The organic layers were combined, dried over anhydrous Na₂SO₄, and filtered. After filtration, the filtrate was concentrated under reduced pressure to afford 4-[4-(tert-butoxycarbonyl)piperazin-1-yl]-2-ethylindazole-7-carboxylic acid (200 mg, 99%) as a solid. LCMS (ES, m/z): 375 [M+H]⁺.

### Synthesis of Intermediate C68

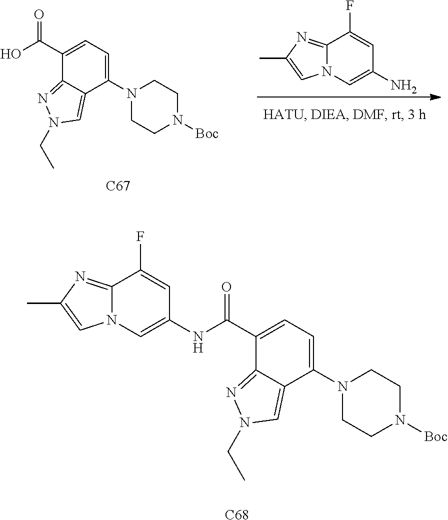 [SMILES: CCn1cc2c(N3CCN(C(=O)OC(C)(C)C)CC3)ccc(C(=O)O)c2n1 ; Cc1cn2cc(N)cc(F)c2n1 ; "C67" ; CCn1cc2c(N3CCN(C(=O)OC(C)(C)C)CC3)ccc(C(=O)Nc3cc(F)c4nc(C)cn4c3)c2n1 ; "C68"]

To a stirred mixture of 4-[4-(tert-butoxycarbonyl)piperazin-1-yl]-2-ethylindazole-7-carboxylic acid (50 mg, 0.134 mmol, 1.00 equiv) and 8-fluoro-2-methylimidazo[1,2-a]pyridin-6-amine (22.06 mg, 0.134 mmol, 1.0 equiv) in DMF (1 mL) was added DIEA (34.5 mg, 0.268 mmol, 2.0 equiv) and HATU (60.9 mg, 0.161 mmol, 1.2 equiv) dropwise at room temperature. The resulting mixture was stirred for 3 h at room temperature. The resulting mixture was diluted with water (3 mL) and extracted with ethyl acetate (3×3 mL). The organic layers were combined, washed with water (3×3 mL), dried over anhydrous Na₂SO₄, and filtered. After filtration, the filtrate was concentrated under reduced pressure to give a residue. The residue was purified by silica gel column chromatography, eluted with EA to afford tert-butyl 4-[2-ethyl-7-({8-fluoro-2-methylimidazo[1,2-a]pyridin-6-yl}carbamoyl)indazol-4-yl]piperazine-1-carboxylate (58 mg, 83%) as a solid. LCMS (ES, m/z): 522 [M+H]⁺.

### Synthesis of Compound 288

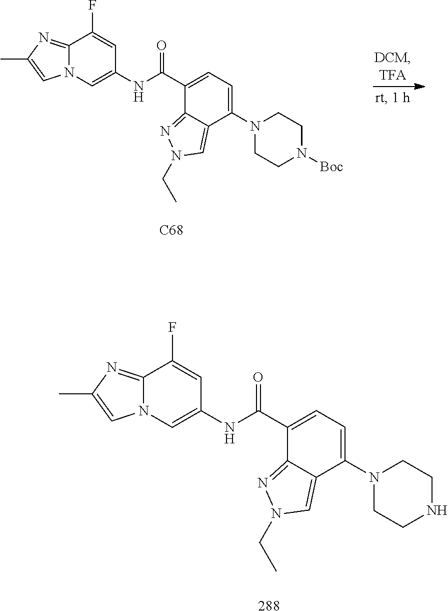

To a stirred solution of tert-butyl 4-[2-ethyl-7-({8-fluoro-2-methylimidazo[1,2-a]pyridin-6-yl}carbamoyl)indazol-4-yl]piperazine-1-carboxylate (58 mg, 1.0 equiv) in DCM (1.8 mL) was added TFA (0.6 mL) dropwise at room temperature. The resulting mixture was stirred for 1 h at room temperature, then concentrated under reduced pressure to give a residue. The residue was purified by Prep-HPLC (Condition 2, Gradient 2) to afford 2-ethyl-N-{8-fluoro-2-methylimidazo[1,2-a]pyridin-6-yl}-4-(piperazin-1-yl)indazole-7-carboxamide (9.8 mg, 21%) as a solid. LCMS (ES, m/z): 422 [M+H]⁺. ¹H NMR (400 MHz, DMSO-d₆) δ 11.11 (s, 1H), 9.22 (d, J=1.7 Hz, 1H), 8.86 (s, 1H), 8.00 (d, J=8.0 Hz, 1H), 7.91 (d, J=3.1 Hz, 1H), 7.32 (dd, J=12.3, 1.7 Hz, 1H), 6.54 (d, J=8.1 Hz, 1H), 4.60 (q, J=7.3 Hz, 2H), 3.44 (t, J=5.1 Hz, 4H), 3.07 (t, J=5.1 Hz, 4H), 2.36 (s, 3H), 1.63 (t, J=7.3 Hz, 3H).

### Example 76: Synthesis of Compound 289

### Synthesis of Intermediate C69

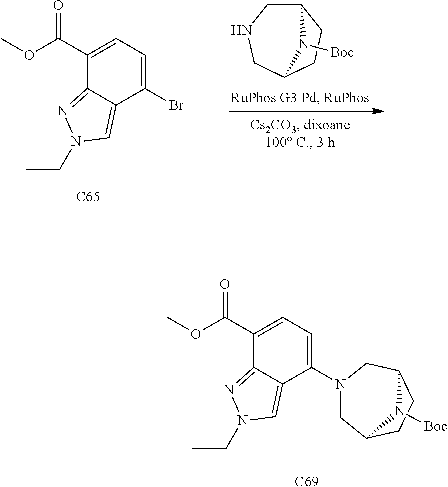 [SMILES: CCn1cc2c(Br)ccc(C(=O)OC)c2n1 ; CC(C)(C)OC(=O)N1[C@@H]2CC[C@H]1CNC2 ; "C65" ; CCn1cc2c(N3C[C@H]4CC[C@@H](C3)N4C(=O)OC(C)(C)C)ccc(C(=O)OC)c2n1 ; "C69"]

To a stirred solution of methyl 4-bromo-2-ethylindazole-7-carboxylate (250 mg, 0.883 mmol, 1.0 equiv) and tert-butyl (1R,5S)-3,8-diazabicyclo[3.2.1]octane-8-carboxylate (374.9 mg, 1.766 mmol, 2.0 equiv) in dioxane (5 mL) was added Cs₂CO₃ (575.40 mg, 1.766 mmol, 2.0 equiv), RuPhos Palladacycle Gen.3 (73.9 mg, 0.088 mmol, 0.1 equiv), and RuPhos (82.4 mg, 0.177 mmol, 0.2 equiv) at room temperature under nitrogen atmosphere. The resulting mixture was stirred for 3 h at 100° C. under nitrogen atmosphere, then concentrated under reduced pressure to give a residue. The residue was purified by silica gel column chromatography, eluted with EA to afford methyl 4-[(1R,5S)-8-(tert-butoxycarbonyl)-3,8-diazabicyclo[3.2.1]octan-3-yl]-2-ethylindazole-7-carboxylate (300 mg, 82%) as a solid. LCMS (ES, m/z): 415 [M+H]⁺.

### Synthesis of Intermediate C70

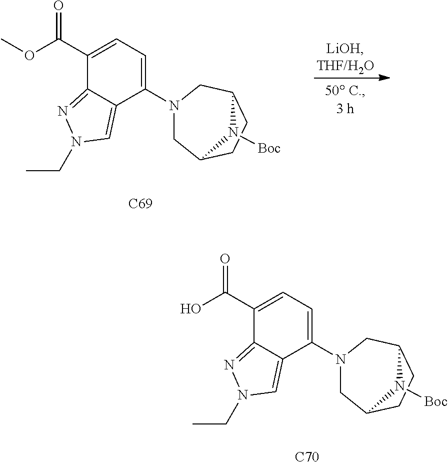 [SMILES: CCn1cc2c(N3C[C@H]4CC[C@@H](C3)N4C(=O)OC(C)(C)C)ccc(C(=O)OC)c2n1 ; "C69" ; CCn1cc2c(N3C[C@H]4CC[C@@H](C3)N4C(=O)OC(C)(C)C)ccc(C(=O)O)c2n1 ; "C70"]

To a stirred solution of methyl 4-[(1R,5S)-8-(tert-butoxycarbonyl)-3,8-diazabicyclo[3.2.1]octan-3-yl]-2-ethylindazole-7-carboxylate (280 mg, 0.676 mmol, 1.0 equiv) in THE (1.4 mL) and water (1.4 mL) was added LiOH·H₂O (113.4 mg, 2.702 mmol, 4.0 equiv) at room temperature. The resulting mixture was stirred for 3 h at 50° C., then concentrated under reduced pressure to give a residue. The resulting mixture was diluted with water (2 mL) and extracted with ethyl acetate (3×3 mL), dried over anhydrous Na₂SO₄, and filtered. After filtration, the filtrate was concentrated under reduced pressure to afford 4-[(1R,5S)-8-(tert-butoxycarbonyl)-3,8-diazabicyclo[3.2.1]octan-3-yl]-2-ethylindazole-7-carboxylic acid (250 mg, 92%) as a solid. LCMS (ES, m/z): 401 [M+H]⁺.

### Synthesis of Intermediate C71

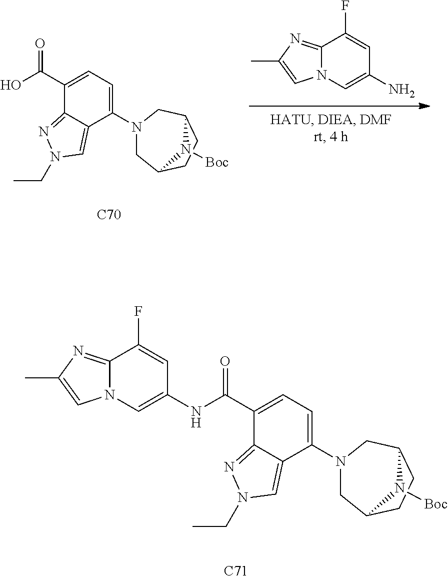 [SMILES: CCn1cc2c(N3C[C@H]4CC[C@@H](C3)N4C(=O)OC(C)(C)C)ccc(C(=O)O)c2n1 ; Cc1cn2cc(N)cc(F)c2n1 ; "C70" ; CCn1cc2c(N3C[C@H]4CC[C@@H](C3)N4C(=O)OC(C)(C)C)ccc(C(=O)Nc3cc(F)c4nc(C)cn4c3)c2n1 ; "C71"]

To a stirred solution of 4-[(1R,5S)-8-(tert-butoxycarbonyl)-3,8-diazabicyclo[3.2.1]octan-3-yl]-2-ethylindazole-7-carboxylic acid (50.0 mg, 0.125 mmol, 1.0 equiv) and 8-fluoro-2-methylimidazo[1,2-a]pyridin-6-amine (20.6 mg, 0.125 mmol, 1.0 equiv) in DMF (1 mL) was added DIEA (48.4 mg, 0.375 mmol, 3.0 equiv) and HATU (85.5 mg, 0.225 mmol, 1.8 equiv) at room temperature. The resulting mixture was stirred for 4 h at room temperature, then diluted with water (3 mL) and extracted with ethyl acetate (3×3 mL). The organic layers were combined, washed with water (3×3 mL), dried over anhydrous Na₂SO₄, and filtered. After filtration, the filtrate was concentrated under reduced pressure to give a residue. The residue was purified by silica gel column chromatography, eluted with EA to afford tert-butyl (1R,5S)-3-[2-ethyl-7-({8-fluoro-2-methylimidazo[1,2-a]pyridin-6-yl}carbamoyl)indazol-4-yl]-3,8-diazabicyclo[3.2.1]octane-8-carboxylate (60 mg, 88%) as a solid. LCMS (ES, m/z): 548 [M+H]⁺.

### Synthesis of Compound 289

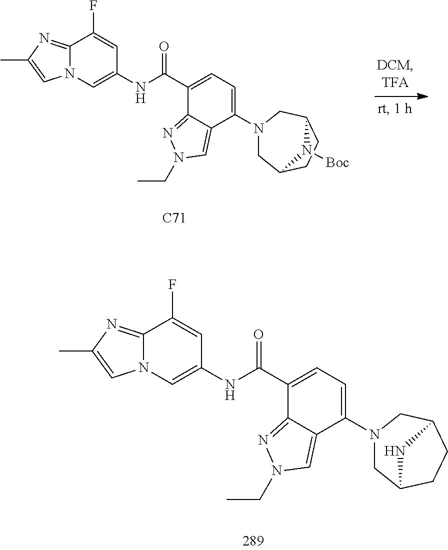 [SMILES: CCn1cc2c(N3C[C@H]4CC[C@@H](C3)N4C(=O)OC(C)(C)C)ccc(C(=O)Nc3cc(F)c4nc(C)cn4c3)c2n1 ; "C71" ; CCn1cc2c(N3C[C@H]4CC[C@@H](C3)N4)ccc(C(=O)Nc3cc(F)c4nc(C)cn4c3)c2n1]

To a stirred solution of tert-butyl (1R,5S)-3-[2-ethyl-7-({8-fluoro-2-methylimidazo[1,2-a]pyridin-6-yl}carbamoyl)indazol-4-yl]-3,8-diazabicyclo[3.2.1]octane-8-carboxylate (60 mg, 1.0 equiv) in DCM (1.8 mL) was added TFA (0.6 mL) dropwise at room temperature. The resulting mixture was stirred for 1 h at room temperature. The resulting mixture was concentrated under vacuum to give a residue. The residue was purified by Prep-HPLC (Condition 2, Gradient 2) to afford 4-[(1R,5S)-3,8-diazabicyclo[3.2.1]octan-3-yl]-2-ethyl-N-{8-fluoro-2-methylimidazo[1,2-a]pyridin-6-yl}indazole-7-carboxamide (17.7 mg, 36%) as a solid. LCMS (ES, m/z): 448 [M+H]⁺. ¹H NMR (300 MHz, DMSO-d₆) δ 11.15 (s, 1H), 9.21 (d, J=1.6 Hz, 1H), 8.87 (s, 1H), 7.99-7.87 (m, 2H), 7.29 (dd, J=12.4, 1.7 Hz, 1H), 6.37 (d, J=8.4 Hz, 1H), 4.58 (q, J=7.2 Hz, 2H), 3.72 (d, J=11.0 Hz, 2H), 3.57 (s, 2H), 3.15 (d, J=10.8 Hz, 2H), 2.52-2.51 (m, 1H), 2.35 (s, 3H), 1.78-1.74 (m, 4H), 1.62 (t, J=7.3 Hz, 3H).

### Example 77: Synthesis of Compound 293

### Synthesis of Intermediate C72

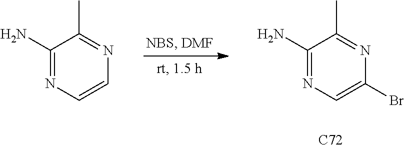

A mixture of 3-methylpyrazin-2-amine (2.00 g, 18.326 mmol, 1.0 equiv) and NBS (3.59 g, 20.159 mmol, 1.1 equiv) in DMF (40 mL) was stirred for 1.5 h at room temperature. The resulting mixture was diluted with water (100 mL) and extracted with ethyl acetate (3×80 mL). The organic layers were combined, washed with brine (3×80 mL), dried over anhydrous Na₂SO₄, and filtered. After filtration, the filtrate was concentrated under reduced pressure to give a residue. The residue was purified by silica gel column chromatography, eluted with PE/EA (1:1) to afford 5-bromo-3-methylpyrazin-2-amine (2.13 g, 62%) as a solid. LCMS (ES, m/z): 188.1 [M+H]⁺.

### Synthesis of Intermediate C73

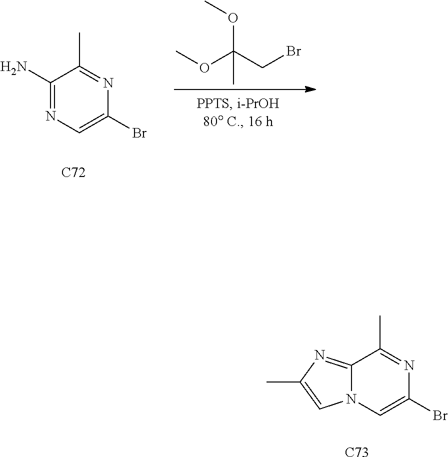

To a stirred mixture of 5-bromo-3-methylpyrazin-2-amine (2.1 g, 11.169 mmol, 1.0 equiv) and 1-bromo-2,2-dimethoxypropane (2.45 g, 13.403 mmol, 1.2 equiv) in isopropanol (41 mL) was added PPTS (196.5 mg, 0.782 mmol, 0.07 equiv) in portions at room temperature. The resulting mixture was stirred for 16 h at 80° C., then diluted with water (40 mL) and extracted with ethyl acetate (4×40 mL). The organic layers were combined, dried over anhydrous Na₂SO₄, and filtered. After filtration, the filtrate was concentrated under reduced pressure to give a residue. The residue was purified by silica gel column chromatography, eluted with EA to afford 6-bromo-2,8-dimethylimidazo[1,2-a]pyrazine (890 mg, 35%) as a solid. LCMS (ES, m/z): 226 [M+H]⁺. Synthesis of Intermediate C74

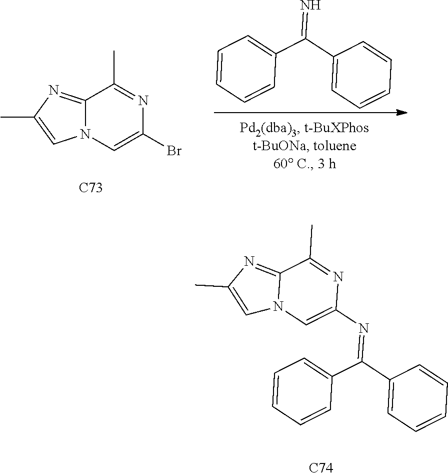 [SMILES: Cc1cn2cc(Br)nc(C)c2n1 ; N=C(c1ccccc1)c1ccccc1 ; "C73" ; Cc1cn2cc(N=C(c3ccccc3)c3ccccc3)nc(C)c2n1 ; "C74"]

To a stirred mixture of 6-bromo-2,8-dimethylimidazo[1,2-a]pyrazine (500.0 mg, 2.212 mmol, 1.0 equiv) and diphenylmethanimine (400.8 mg, 2.212 mmol, 1.0 equiv) in toluene (7.5 mL) was added t-BuONa (637.6 mg, 6.636 mmol, 3.0 equiv), Pd₂(dba)₃ (202.5 mg, 0.221 mmol, 0.1 equiv), and t-BuXPhos (187.8 mg, 0.442 mmol, 0.2 equiv) at room temperature under nitrogen atmosphere. The resulting mixture was stirred for 3 h at 60° C. under nitrogen atmosphere, then concentrated under vacuum to give a residue. The residue was purified by silica gel column chromatography, eluted with EA to afford N-{2,8-dimethylimidazo[1,2-a]pyrazin-6-yl}-1,1-diphenylmethanimine (630 mg, 87%) as a oil. LCMS (ES, m/z): 327 [M+H]⁺.

### Synthesis of Intermediate C75

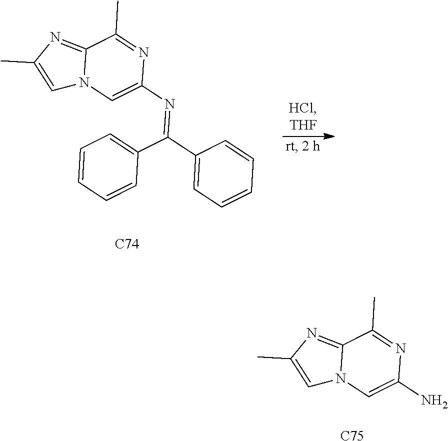 [SMILES: Cc1cn2cc(N=C(c3ccccc3)c3ccccc3)nc(C)c2n1 ; "C74" ; Cc1cn2cc(N)nc(C)c2n1 ; "C75"]

To a stirred solution of N-{2,8-dimethylimidazo[1,2-a]pyrazin-6-yl}-1,1-diphenylmethanimine (620 mg, 1.899 mmol, 1.0 equiv) in THE (12 mL) was added HCl (6 mL, con) dropwise at room temperature. The resulting mixture was stirred for 2 h at room temperature. The resulting mixture was diluted with water (15 mL), basified to pH 8 with saturated Na₂CO₃ (aq.), and extracted with ethyl acetate (3×20 mL). The organic layers were combined, dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford 2,8-dimethylimidazo[1,2-a]pyrazin-6-amine (160 mg, 52%) as a solid. LCMS (ES, m/z): 163 [M+H]⁺.

### Synthesis of Intermediate C76

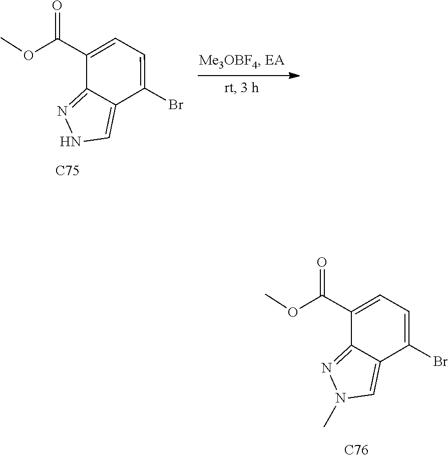

To a stirred solution of methyl 4-bromo-2H-indazole-7-carboxylate (500 mg, 1.960 mmol, 1.00 equiv) and in ethyl acetate (7.5 mL) was added Me₃OBF₄ (1449.67 mg, 9.800 mmol, 5.0 equiv) at room temperature. The resulting mixture was stirred for 3 h at room temperature, then diluted with water (10 mL) and extracted with ethyl acetate (3×10 mL). The organic layers were combined, washed with water (2×10 mL), dried over anhydrous Na₂SO₄, and filtered. After filtration, the filtrate was concentrated under reduced pressure to give a residue. The residue was purified by silica gel column chromatography, eluted with EA to afford methyl 4-bromo-2-methylindazole-7-carboxylate (410 mg, 78%) as a solid. LCMS (ES, m/z): 269 [M+H]⁺.

### Synthesis of Intermediate C77

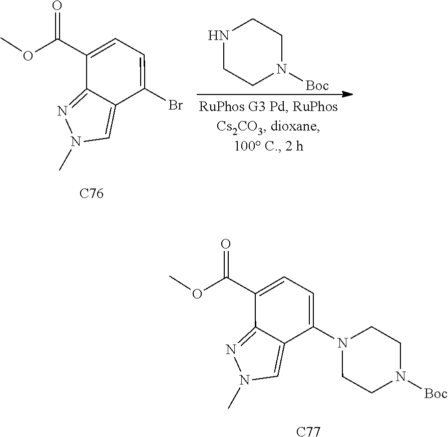

To a stirred mixture of methyl 4-bromo-2-methylindazole-7-carboxylate (410.0 mg, 1.524 mmol, 1.0 equiv) and tert-butyl piperazine-1-carboxylate (567.6 mg, 3.048 mmol, 2.0 equiv) in dioxane (8.2 mL) was added Cs₂CO₃ (1.49 g, 4.572 mmol, 3.0 equiv), RuPhos Palladacycle Gen.3 (127.4 mg, 0.152 mmol, 0.1 equiv), and RuPhos (142.2 mg, 0.305 mmol, 0.2 equiv) at room temperature under nitrogen atmosphere. The resulting mixture was stirred for 2 h at 100° C. under nitrogen atmosphere, then concentrated under vacuum to give a residue. The residue was purified by silica gel column chromatography, eluted with PE/EA (1:1) to afford methyl 4-[4-(tert-butoxycarbonyl)piperazin-1-yl]-2-methylindazole-7-carboxylate (700 mg, 98%) as a solid. LCMS (ES, m/z): 375 [M+H]⁺.

### Synthesis of Intermediate C78

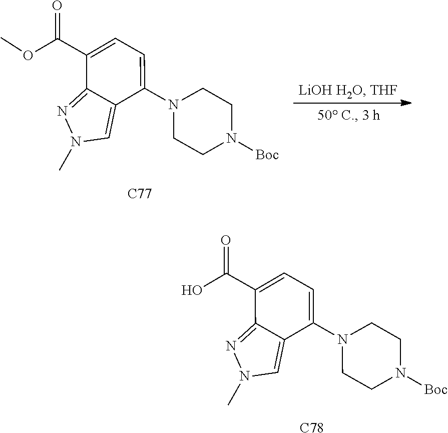

To a stirred mixture of methyl 4-[4-(tert-butoxycarbonyl)piperazin-1-yl]-2-methylindazole-7-carboxylate (300 mg, 0.801 mmol, 1.00 equiv) in THE (1.5 mL) and water (1.5 mL) was added LiOH·H₂O (95.9 mg, 4.005 mmol, 5 equiv) at room temperature. The resulting mixture was stirred for 3 h at 50° C., then concentrated under vacuum. The resulting mixture was diluted with water (6 mL), acidified to pH 7 with concentrated HCl, and extracted with ethyl acetate (4×10 mL). The organic layers were combined, dried over anhydrous Na₂SO₄, and filtered. After filtration, the filtrate was concentrated under reduced pressure to afford 4-[4-(tert-butoxycarbonyl)piperazin-1-yl]-2-methylindazole-7-carboxylic acid (200 mg, 69%) as a solid. LCMS (ES, m/z): 361 [M+H]⁺.

### Synthesis of Intermediate C79

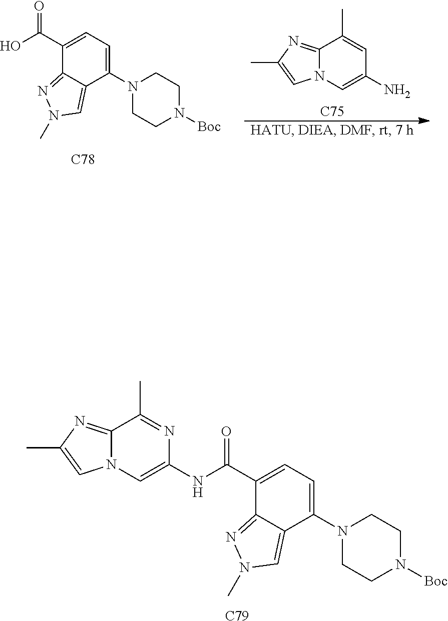

To a stirred mixture of 4-[4-(tert-butoxycarbonyl)piperazin-1-yl]-2-methylindazole-7-carboxylic acid (80.0 mg, 0.222 mmol, 1.0 equiv) and 2,8-dimethylimidazo[1,2-a]pyrazin-6-amine (36.0 mg, 0.222 mmol, 1.0 equiv) in DMF (1.6 mL) was added DIEA (86.1 mg, 0.666 mmol, 3.0 equiv) and HATU (151.9 mg, 0.400 mmol, 1.8 equiv) at room temperature. The resulting mixture was stirred for 7 h at room temperature, then diluted with water (3 mL) and extracted with ethyl acetate (3×5 mL). The organic layers were combined, washed with water (3×5 mL), dried over anhydrous Na₂SO₄, and filtered. After filtration, the filtrate was concentrated under reduced pressure to give a residue. The residue was purified by silica gel column chromatography, eluted with EA to afford tert-butyl 4-[7-({2,8-dimethylimidazo[1,2-a]pyrazin-6-yl}carbamoyl)-2-methylindazol-4-yl]piperazine-1-carboxylate (60 mg, 54%) as a solid. LCMS (ES, m/z): 505 [M+H]⁺. Synthesis of Compound 293

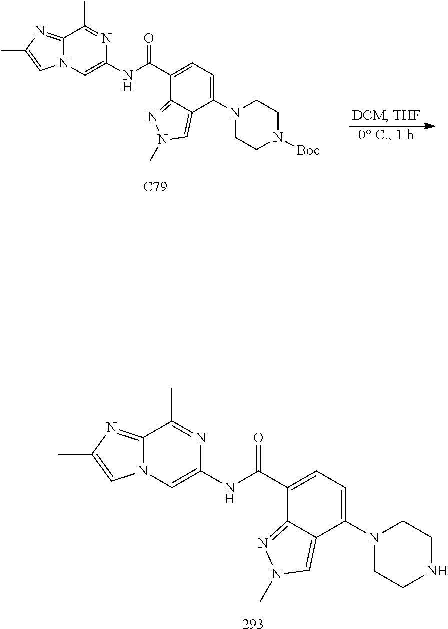

To a stirred solution of tert-butyl 4-[7-({2,8-dimethylimidazo[1,2-a]pyrazin-6-yl}carbamoyl)-2-methylindazol-4-yl]piperazine-1-carboxylate (60 mg, 0.119 mmol, 1 equiv) in DCM (0.6 mL) was added TFA (1.8 mL) dropwise at 0° C. The resulting mixture was stirred for 1 h at 0° C., then concentrated under vacuum to give a residue. The residue was purified by Prep-HPLC (Condition 2, Gradient 2) to afford N-{2,8-dimethylimidazo[1,2-a]pyrazin-6-yl}-2-methyl-4-(piperazin-1-yl)indazole-7-carboxamide (5.9 mg, 12%) as a solid. LCMS (ES, m/z): 405 [M+H]⁺. ¹H NMR (300 MHz, DMSO-d₆) δ 11.27 (s, 1H), 9.30 (s, 1H), 8.81 (s, 1H), 8.07-7.95 (m, 2H), 6.50 (d, J=8.2 Hz, 1H), 4.28 (s, 3H), 3.37 (s, 4H), 2.93 (d, J=4.7 Hz, 4H), 2.73 (s, 3H), 2.39 (s, 3H).

### Example 78: Synthesis of Compound 291

### Synthesis of Compound 291

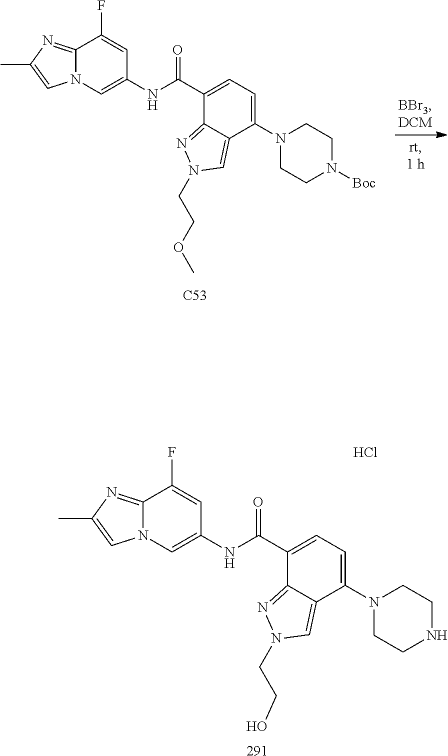

To a stirred solution of tert-butyl 4-[7-({8-fluoro-2-methylimidazo[1,2-a]pyridin-6-yl}carbamoyl)-2-(2-methoxyethyl)indazol-4-yl]piperazine-1-carboxylate (90.0 mg, 0.163 mmol, 1.0 equiv) in DCM (1.8 mL) was added BBr₃ (163.5 mg, 0.652 mmol, 4.0 equiv) dropwise at 0° C. The resulting mixture was concentrated under vacuum to give a residue. The residue was purified by Prep-HPLC (Condition 1, Gradient 1) to afford N-{8-fluoro-2-methylimidazo[1,2-a]pyridin-6-yl}-2-(2-hydroxyethyl)-4-(piperazin-1-yl)indazole-7-carboxamide hydrochloride (4.3 mg, 6%) as a solid. LCMS (ES, m/z): 438 [M+H]⁺. ¹H NMR (400 MHz, DMSO-d₆) δ 11.43 (s, 1H), 9.63 (d, J=1.6 Hz, 1H), 9.38-9.37 (m, 2H), 8.93 (s, 1H), 8.33-8.28 (m, 1H), 8.10 (d, J=11.6 Hz, 1H), 8.05 (d, J=8.0 Hz, 1H), 6.63 (d, J=8.1 Hz, 1H), 4.63 (t, J=5.4 Hz, 2H), 4.02 (t, J=5.4 Hz, 2H), 3.66 (t, J=5.1 Hz, 4H), 3.33-3.32 (m, 4H), 2.51 (s, 3H).

### Example 79: Synthesis of Compound 292

### Synthesis of Intermediate C80

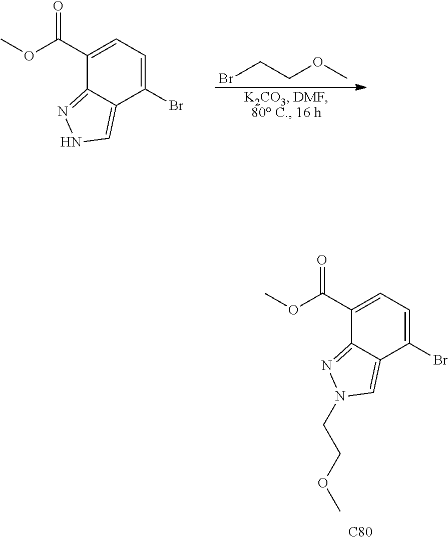

To a stirred mixture of methyl 4-bromo-2H-indazole-7-carboxylate (600.0 mg, 2.352 mmol, 1.0 equiv) and K₂CO₃ (650.0 mg, 4.704 mmol, 2.0 equiv) in DMF (6 mL) was added 2-bromoethyl methyl ether (490.4 mg, 3.528 mmol, 1.5 equiv) at room temperature. The resulting mixture was stirred for 16 h at 80° C., then diluted with water (15 mL) and extracted with ethyl acetate (3×20 mL). The organic layers were combined, washed with water (3×15 mL), dried over anhydrous Na₂SO₄, and filtered. After filtration, the filtrate was concentrated under reduced pressure to give a residue. The residue was purified by silica gel column chromatography, eluted with PE/EA (1:1) to afford methyl 4-bromo-2-(2-methoxyethyl)indazole-7-carboxylate (200 mg, 27%) as a solid. LCMS (ES, m/z): 312 [M+H]⁺.

### Synthesis of Intermediate C81

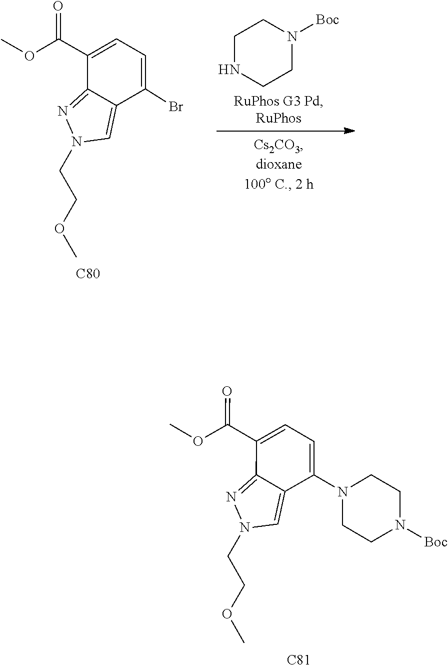 [SMILES: COCCn1cc2c(Br)ccc(C(=O)OC)c2n1 ; CC(C)(C)OC(=O)N1CCNCC1 ; "C80" ; COCCn1cc2c(N3CCN(C(=O)OC(C)(C)C)CC3)ccc(C(=O)OC)c2n1 ; "C81"]

To a stirred mixture of methyl 4-bromo-2-(2-methoxyethyl)indazole-7-carboxylate (200.0 mg, 0.639 mmol, 1.0 equiv) and tert-butyl piperazine-1-carboxylate (237.9 mg, 1.278 mmol, 2.0 equiv) in dioxane (4 mL) was added Cs₂CO₃ (624.3 mg, 1.917 mmol, 3.0 equiv), RuPhos Palladacycle Gen.3 (53.4 mg, 0.064 mmol, 0.1 equiv), and RuPhos (59.61 mg, 0.128 mmol, 0.2 equiv) at room temperature under nitrogen atmosphere. The resulting mixture was stirred for 2 h at 100° C. under nitrogen atmosphere, then concentrated under vacuum to give a residue. The residue was purified by silica gel column chromatography, eluted with EA to afford methyl 4-[4-(tert-butoxycarbonyl)piperazin-1-yl]-2-(2-methoxyethyl)indazole-7-carboxylate (320 mg, 99%) as an oil. LCMS (ES, m/z): 419 [M+H]⁺.

### Synthesis of Intermediate C82

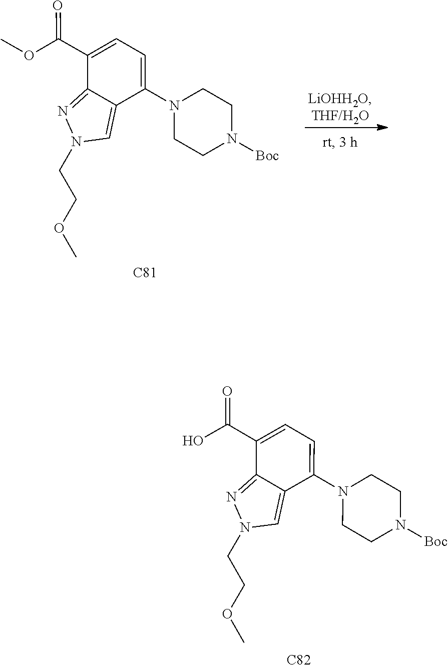 [SMILES: COCCn1cc2c(N3CCN(C(=O)OC(C)(C)C)CC3)ccc(C(=O)OC)c2n1 ; "C81" ; COCCn1cc2c(N3CCN(C(=O)OC(C)(C)C)CC3)ccc(C(=O)O)c2n1 ; "C82"]

To a stirred mixture of methyl 4-[4-(tert-butoxycarbonyl)piperazin-1-yl]-2-(2-methoxyethyl)indazole-7-carboxylate (320.0 mg, 0.765 mmol, 1.0 equiv) in THE (1.6 mL) and water (1.6 mL) was added LiOH·H₂O (91.6 mg, 3.825 mmol, 5.0 equiv) at room temperature. The resulting mixture was stirred for 3 h at room temperature, then concentrated under vacuum. The resulting mixture was diluted with water (5 mL), acidified to pH 6 with HCl (1 N), and extracted with ethyl acetate (4×30 mL). The organic layers were combined, dried over anhydrous Na₂SO₄, and filtered. After filtration, the filtrate was concentrated under reduced pressure. to afford 4-[4-(tert-butoxycarbonyl)piperazin-1-yl]-2-(2-methoxyethyl)indazole-7-carboxylic acid (220 mg, 71%) as a solid. LCMS (ES, m/z): 405 [M+H]⁺.

### Synthesis of Intermediate C83

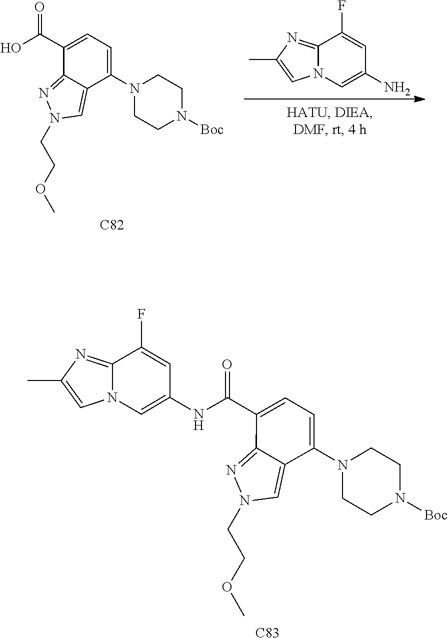 [SMILES: COCCn1cc2c(N3CCN(C(=O)OC(C)(C)C)CC3)ccc(C(=O)O)c2n1 ; Cc1cn2cc(N)cc(F)c2n1 ; "C82" ; COCCn1cc2c(N3CCN(C(=O)OC(C)(C)C)CC3)ccc(C(=O)Nc3cc(F)c4nc(C)cn4c3)c2n1 ; "C83"]

To a stirred mixture of 4-[4-(tert-butoxycarbonyl)piperazin-1-yl]-2-(2-methoxyethyl)indazole-7-carboxylic acid (220.0 mg, 0.544 mmol, 1.0 equiv) and 8-fluoro-2-methylimidazo[1,2-a]pyridin-6-amine (89.8 mg, 0.544 mmol, 1.0 equiv) in DMF (4.4 mL) was added DIEA (210.9 mg, 1.632 mmol, 3.0 equiv) and HATU (372.3 mg, 0.979 mmol, 1.8 equiv) at room temperature. The resulting mixture was stirred for 4 h at room temperature, then diluted with water (12 mL) and extracted with ethyl acetate (3×10 mL). The organic layers were combined, washed with brine (3×10 mL), dried over anhydrous Na₂SO₄, and filtered. After filtration, the filtrate was concentrated under reduced pressure to give a residue. The residue was purified by silica gel column chromatography, eluted with EA to afford tert-butyl 4-[7-({8-fluoro-2-methylimidazo[1,2-a]pyridin-6-yl}carbamoyl)-2-(2-methoxyethyl)indazol-4-yl]piperazine-1-carboxylate (170 mg, 34%) as a solid. LCMS (ES, m/z): 552 [M+H]⁺.

### Synthesis of Compound 292

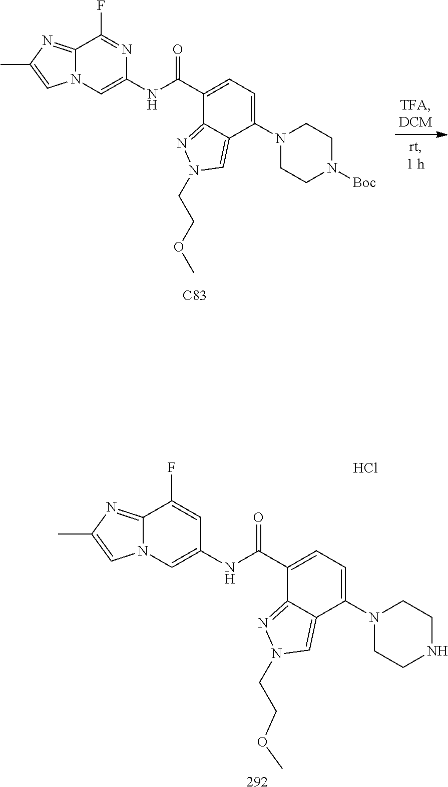

To a stirred solution of tert-butyl 4-[7-({8-fluoro-2-methylimidazo[1,2-a]pyridin-6-yl}carbamoyl)-2-(2-methoxyethyl)indazol-4-yl]piperazine-1-carboxylate (70.0 mg, 0.076 mmol, 1.0 equiv) in DCM (1.5 mL) was added TFA (0.5 mL) dropwise at room temperature. The resulting mixture was stirred for 1 h at room temperature, then concentrated under vacuum to give a residue. The residue was purified by Prep-HPLC (Condition 1, Gradient 1) to afford N-{8-fluoro-2-methylimidazo[1,2-a]pyridin-6-yl}-2-(2-methoxyethyl)-4-(piperazin-1-yl)indazole-7-carboxamide hydrochloride (17.9 mg, 52%) as a solid. LCMS (ES, m/z): 452 [M+H]⁺. ¹H NMR (400 MHz, DMSO-d₆) δ 11.40 (s, 1H), 9.65 (d, J=1.5 Hz, 1H), 9.50-9.49 (m, 2H), 8.97 (s, 1H), 8.31 (s, 1H), 8.12 (d, J=11.6 Hz, 1H), 8.05 (d, J=8.0 Hz, 1H), 6.63 (d, J=8.1 Hz, 1H), 4.75 (t, J=5.2 Hz, 2H), 3.98 (t, J=5.2 Hz, 2H), 3.66 (t, J=5.0 Hz, 4H), 3.32-3.31 (m, 4H), 3.29 (s, 3H), 2.50 (s, 3H).

### Example 80: Synthesis of Compound 296

### Synthesis of Intermediate C84

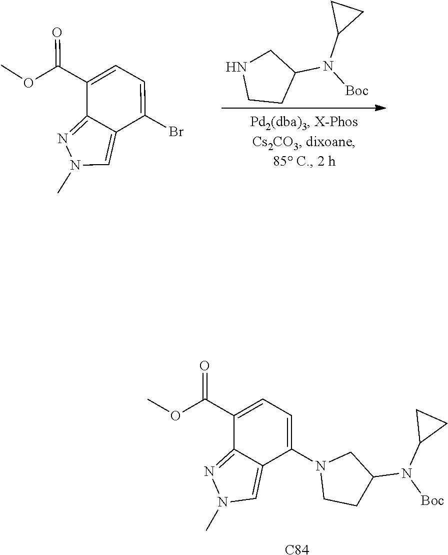

To a stirred mixture of methyl 4-bromo-2-methylindazole-7-carboxylate (220.0 mg, 0.818 mmol, 1.0 equiv) and tert-butyl N-cyclopropyl-N-(pyrrolidin-3-yl)carbamate (370.1 mg, 1.636 mmol, 2.0 equiv) in dioxane (4 mL) was added Cs₂CO₃ (532.7 mg, 1.636 mmol, 2.0 equiv), Pd₂(dba)₃ (74.8 mg, 0.082 mmol, 0.1 equiv), and X-Phos (77.9 mg, 0.164 mmol, 0.2 equiv) at room temperature under nitrogen atmosphere. The resulting mixture was stirred for 2 h at 85° C. under nitrogen atmosphere, then concentrated under reduced pressure to give a residue. The residue was purified by silica gel column chromatography, eluted with CH₂Cl₂/MeOH (10:1) to afford methyl 4-{3-[(tert-butoxycarbonyl)(cyclopropyl)amino]pyrrolidin-1-yl}-2-methylindazole-7-carboxylate (290 mg, 86%) as a solid. LCMS (ES, m/z): 415 [M+H]⁺.

### Synthesis of Intermediate C85

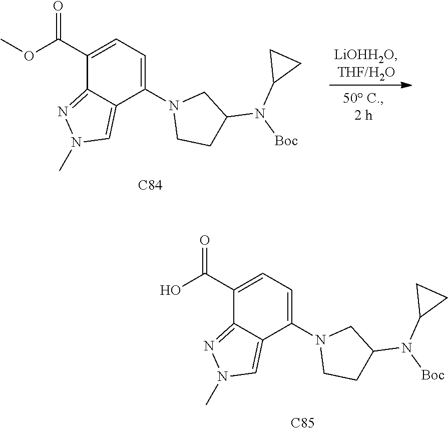 [SMILES: COC(=O)c1ccc(N2CCC(N(C(=O)OC(C)(C)C)C3CC3)C2)c2cn(C)nc12 ; "C84" ; Cn1cc2c(N3CCC(N(C(=O)OC(C)(C)C)C4CC4)C3)ccc(C(=O)O)c2n1 ; "C85"]

To a stirred solution of methyl 4-{3-[(tert-butoxycarbonyl)(cyclopropyl)amino]pyrrolidin-1-yl}-2-methylindazole-7-carboxylate (100.0 mg, 0.241 mmol, 1.0 equiv) in THE (1.25 mL) and water (1.25 mL) was added LiOH·H₂O (81.0 mg, 1.928 mmol, 8.0 equiv) at room temperature. The resulting mixture was stirred for 2 h at 50° C., then concentrated under vacuum. The resulting mixture was diluted with water (2 mL), acidified to pH 7 with HCl (1 N), and extracted with ethyl acetate (3×2 mL). The organic layers were combined, dried over anhydrous Na₂SO₄, and filtered. After filtration, the filtrate was concentrated under reduced pressure to afford 4-{3-[(tert-butoxycarbonyl)(cyclopropyl)amino]pyrrolidin-1-yl}-2-methylindazole-7-carboxylic acid (80 mg, 83%) as a solid. LCMS (ES, m/z): 401 [M+H]⁺.

### Synthesis of Intermediate C86

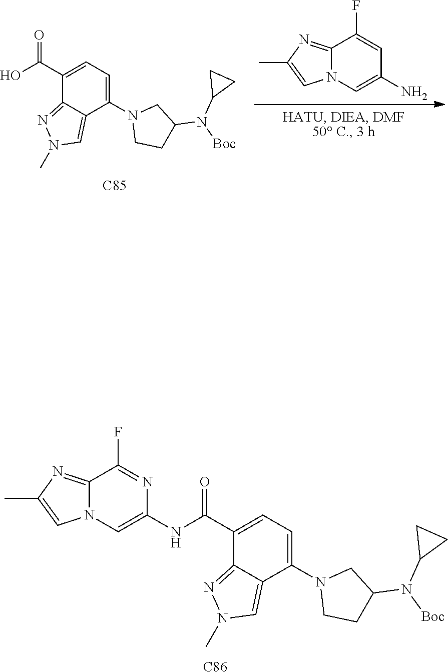

To a stirred solution of 4-{3-[(tert-butoxycarbonyl)(cyclopropyl)amino]pyrrolidin-1-yl}-2-methylindazole-7-carboxylic acid (80.0 mg, 0.200 mmol, 1.0 equiv) and 8-fluoro-2-methylimidazo[1,2-a]pyridin-6-amine (33.0 mg, 0.200 mmol, 1.0 equiv) in DMF (1.4 mL) was added DIEA (77.5 mg, 0.600 mmol, 3.0 equiv) and HATU (113.9 mg, 0.300 mmol, 1.5 equiv) at room temperature. The resulting mixture was stirred for 3 h at 50° C., then diluted with water (1 mL) and extracted with ethyl acetate (3×2 mL). The organic layers were combined, washed with water (3×2 mL), dried over anhydrous Na₂SO₄, and filtered. After filtration, the filtrate was concentrated under reduced pressure to give a residue. The residue was purified by silica gel column chromatography, eluted with EA (100%) to afford tert-butyl N-cyclopropyl-N-{1-[7-({8-fluoro-2-methylimidazo[1,2-a]pyridin-6-yl}carbamoyl)-2-methylindazol-4-yl]pyrrolidin-3-yl}carbamate (80 mg, 73%) as an oil. LCMS (ES, m/z): 548 [M+H]⁺.

### Synthesis of Compound 296

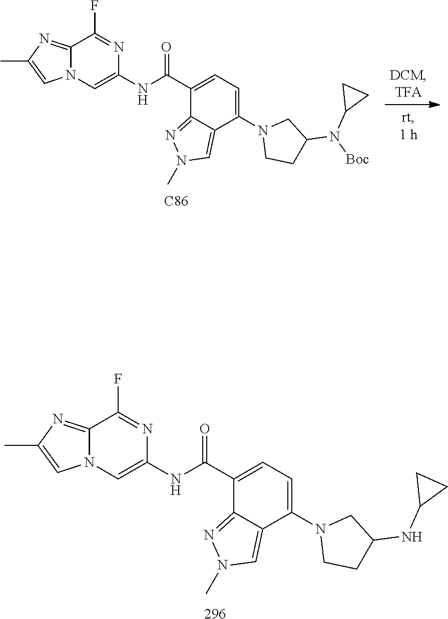

To a stirred solution of tert-butyl N-cyclopropyl-N-{1-[7-({8-fluoro-2-methylimidazo[1,2-a]pyridin-6-yl}carbamoyl)-2-methylindazol-4-yl]pyrrolidin-3-yl}carbamate (80.0 mg, 0.088 mmol, 1.0 equiv) in DCM (2.4 mL) was added TFA (0.8 mL) dropwise at 0° C. The resulting mixture was concentrated under vacuum to give a residue. The residue was purified by Prep-HPLC (Condition 2, Gradient 2) to afford 4-[3-(cyclopropylamino)pyrrolidin-1-yl]-N-{8-fluoro-2-methylimidazo[1,2-a]pyridin-6-yl}-2-methylindazole-7-carboxamide (9.8 mg, 25%) as a solid. LCMS (ES, m/z): 448 [M+H]⁺. ¹H NMR (400 MHz, DMSO-d₆) δ 11.01 (s, 1H), 9.19 (d, J=1.6 Hz, 1H), 8.82 (s, 1H), 7.96-7.86 (m, 2H), 7.31 (dd, J=12.4, 1.7 Hz, 1H), 6.01 (d, J=8.4 Hz, 1H), 4.27 (s, 3H), 3.84-3.71 (m, 2H), 3.64 (d, J=8.1 Hz, 1H), 3.56 (q, J=5.4 Hz, 1H), 3.51-3.44 (m, 1H), 2.35 (s, 3H), 2.17 (dt, J=7.2, 4.5 Hz, 2H), 1.98 (dq, J=12.6, 6.4 Hz, 1H), 0.49-0.36 (m, 2H), 0.32-0.20 (m, 2H).

### Example 81: Synthesis of Compound 298

### Synthesis of Intermediate C87

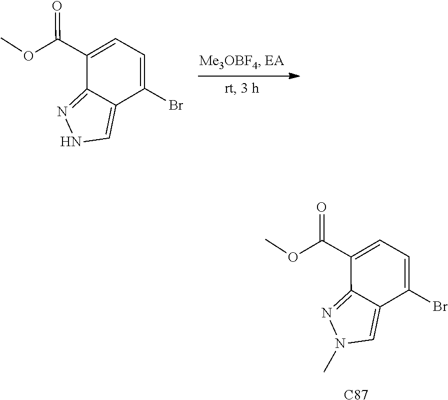

To a stirred solution of methyl 4-bromo-2H-indazole-7-carboxylate (0.5 g, 1.960 mmol, 1.0 equiv) in ethyl acetate (7.5 mL) was added tetrafluoroboranuide; trimethyloxidanium (1.45 g, 9.800 mmol, 5.0 equiv) at room temperature. The resulting mixture was stirred for 3 h at room temperature, then diluted with water (10 mL) and extracted with ethyl acetate (3×10 mL). The organic layers were combined, washed with water (2×10 mL), dried over anhydrous Na₂SO₄, and filtered. After filtration, the filtrate was concentrated under reduced pressure to give a residue. The residue was purified by silica gel column chromatography, eluted with EA to afford methyl 4-bromo-2-methylindazole-7-carboxylate (560 mg, 99%) as a solid. LCMS (ES, m/z): 269 [M+H]⁺.

### Synthesis of Intermediate C88

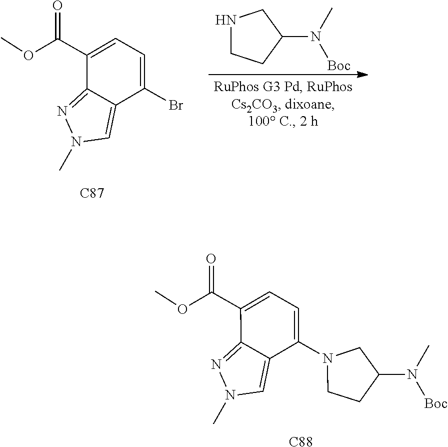

To a stirred solution of methyl 4-bromo-2-methylindazole-7-carboxylate (200.0 mg, 0.743 mmol, 1.0 equiv) and tert-butyl N-methyl-N-(pyrrolidin-3-yl)carbamate (297.7 mg, 1.486 mmol, 2.0 equiv) in dioxane (4 mL) was added Cs₂CO₃ (726.5 mg, 2.229 mmol, 3.0 equiv), RuPhos (69.4 mg, 0.149 mmol, 0.2 equiv), and RuPhos Palladacycle Gen.3 (62.2 mg, 0.074 mmol, 0.1 equiv) at room temperature under nitrogen atmosphere. The resulting mixture was stirred for 2 h at 100° C. under nitrogen atmosphere, then concentrated under reduced pressure to give a residue. The residue was purified by silica gel column chromatography, eluted with CH₂Cl₂/MeOH (10:1) to afford methyl 4-{3-[(tert-butoxycarbonyl)(methyl)amino]pyrrolidin-1-yl}-2-methylindazole-7-carboxylate (280 mg, 97%) as a solid. LCMS (ES, m/z): 389 [M+H]⁺.

### Synthesis of Intermediate C89

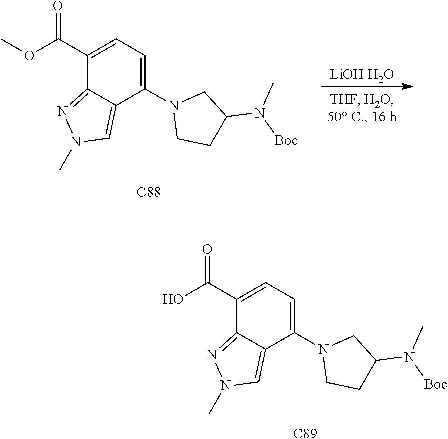 [SMILES: COC(=O)c1ccc(N2CCC(N(C)C(=O)OC(C)(C)C)C2)c2cn(C)nc12 ; "LiOH H2O" ; "C88" ; CN(C(=O)OC(C)(C)C)C1CCN(c2ccc(C(=O)O)c3nn(C)cc23)C1 ; "C89"]

To a stirred solution of methyl 4-{3-[(tert-butoxycarbonyl)(methyl)amino]pyrrolidin-1-yl}-2-methylindazole-7-carboxylate (290.0 mg, 0.747 mmol, 1.0 equiv) in THF (3.7 mL) was added water (3.7 mL) and lithiumol hydrate (156.6 mg, 3.735 mmol, 5.0 equiv) at room temperature. The resulting mixture was stirred for 16 h at 50° C. The resulting mixture was concentrated under vacuum, diluted with water (10 mL), acidified to pH 7 with concentrated HCl, and extracted with ethyl acetate (3×10 mL). The organic layers were combined, washed with brine (3×10 mL), dried over anhydrous Na₂SO₄, and filtered. After filtration, the filtrate was concentrated under reduced pressure to afford 4-{3-[(tert-butoxycarbonyl)(methyl)amino]pyrrolidin-1-yl}-2-methylindazole-7-carboxylic acid (270 mg, 97%) as a solid. LCMS (ES, m/z): 375 [M+H]⁺.

### Synthesis of Intermediate C90

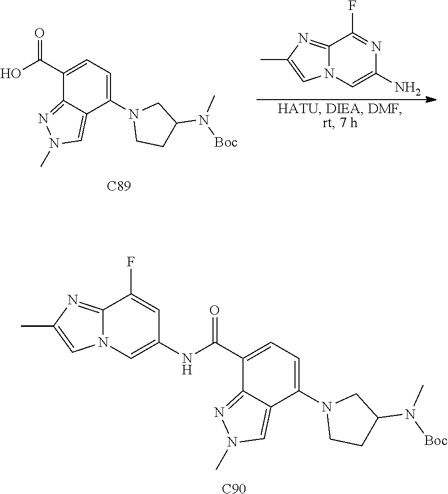

To a stirred solution of 4-{3-[(tert-butoxycarbonyl)(methyl)amino]pyrrolidin-1-yl}-2-methylindazole-7-carboxylic acid (70.0 mg, 0.187 mmol, 1.0 equiv) in DMF (1.4 mL) was added HATU (106.6 mg, 0.280 mmol, 1.5 equiv), DIEA (72.5 mg, 0.561 mmol, 3.0 equiv), and 8-fluoro-2-methylimidazo[1,2-a]pyridin-6-amine (30.9 mg, 0.187 mmol, 1 equiv) in portions at room temperature. The resulting mixture was stirred for 7 h at room temperature, then diluted with water (5 mL) and extracted with ethyl acetate (3×5 mL). The organic layers were combined, washed with water (3×5 mL), dried over anhydrous Na₂SO₄, and filtered. After filtration, the filtrate was concentrated under reduced pressure to give a residue. The residue was purified by silica gel column chromatography, eluted with EA (100%) to afford tert-butyl N-{1-[7-({8-fluoro-2-methylimidazo[1,2-a]pyridin-6-yl}carbamoyl)-2-methylindazol-4-yl]pyrrolidin-3-yl}-N-methylcarbamate (77 mg, 79%) as a solid. LCMS (ES, m/z): 522 [M+H]⁺.

### Synthesis of Compound 298

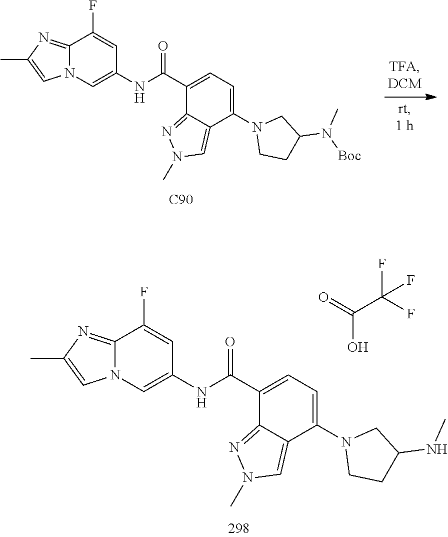

To a stirred solution of tert-butyl N-{1-[7-({8-fluoro-2-methylimidazo[1,2-a]pyridin-6-yl}carbamoyl)-2-methylindazol-4-yl]pyrrolidin-3-yl}-N-methylcarbamate (77.0 mg, 0.148 mmol, 1.0 equiv) in DCM (2.1 mL) was added TFA (0.7 mL) dropwise at 0° C. The resulting mixture was stirred for 1 h at room temperature, then concentrated under vacuum to give a residue. The residue was purified by Prep-HPLC (Condition 3, Gradient 2) to afford N-{8-fluoro-2-methylimidazo[1,2-a]pyridin-6-yl}-2-methyl-4-[3-(methylamino)pyrrolidin-1-yl]indazole-7-carboxamide trifluoroacetic acid salt (30.3 mg, 49%) as a solid. LCMS (ES, m/z): 422 [M+H]⁺. ¹H NMR (400 MHz, DMSO-d₆) δ 11.18 (s, 1H), 9.39 (s, 1H), 8.90-8.89 (m, 3H), 8.08 (s, 1H), 7.99 (d, J=8.2 Hz, 1H), 7.70 (d, J=11.9 Hz, 1H), 6.13 (d, J=8.4 Hz, 1H), 4.31 (s, 3H), 3.99-3.97 (m, 2H), 3.89-3.73 (m, 3H), 2.73-2.67 (m, 3H), 2.49-2.48 (m, 1H), 2.42 (s, 3H), 2.28-2.25 (m, 1H).

### Example 82: Synthesis of Compound 301

### Synthesis of Intermediate C91

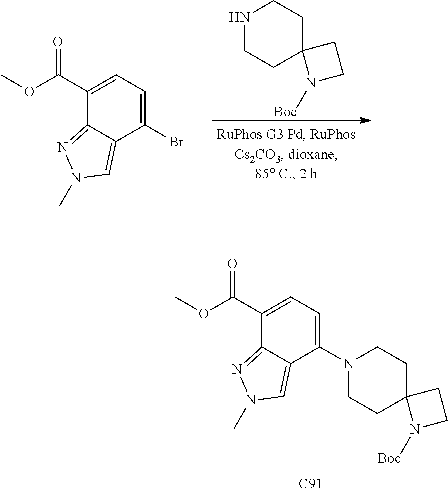 [SMILES: COC(=O)c1ccc(Br)c2cn(C)nc12 ; CC(C)(C)OC(=O)N1CCC12CCNCC2 ; COC(=O)c1ccc(N2CCC3(CC2)CCN3C(=O)OC(C)(C)C)c2cn(C)nc12 ; "C91"]

To a stirred mixture of methyl 4-bromo-2-methylindazole-7-carboxylate (120.0 mg, 0.446 mmol, 1.0 equiv) and tert-butyl 1,7-diazaspiro[3.5]nonane-1-carboxylate (201.8 mg, 0.892 mmol, 2.0 equiv) in dioxane (2.5 mL) was added Cs₂CO₃ (435.9 mg, 1.338 mmol, 3.0 equiv), RuPhos (41.6 mg, 0.089 mmol, 0.2 equiv), and RuPhos Palladacycle Gen.3 (37.3 mg, 0.045 mmol, 0.1 equiv) in portions at room temperature under nitrogen atmosphere. The resulting mixture was stirred for 2 h at 85° C. under nitrogen atmosphere, then concentrated under vacuum to give a residue. The residue was purified by silica gel column chromatography, eluted with CH₂Cl₂/MeOH (10:1) to afford tert-butyl 7-[7-(methoxycarbonyl)-2-methylindazol-4-yl]-1,7-diazaspiro[3.5]nonane-1-carboxylate (180 mg, 97%) as a solid. LCMS (ES, m/z): 415 [M+H]⁺.

### Synthesis of Intermediate C92

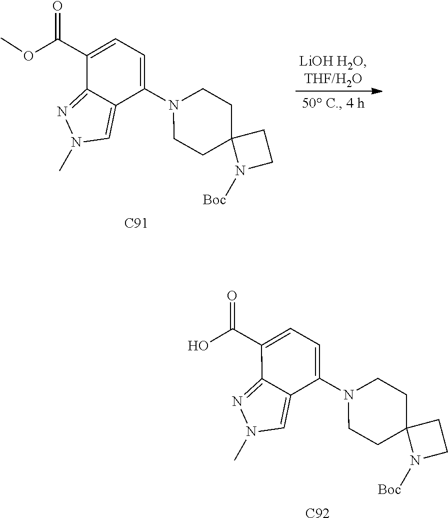

To a stirred mixture of tert-butyl 7-[7-(methoxycarbonyl)-2-methylindazol-4-yl]-1,7-diazaspiro[3.5]nonane-1-carboxylate (180.0 mg, 0.434 mmol, 1.0 equiv) in THE (2.5 mL) and water (2.5 mL) was added LiOH·H₂O (52.0 mg, 2.170 mmol, 5.0 equiv) at room temperature. The resulting mixture was stirred for 4 h at 50° C., then concentrated under vacuum, diluted with water (5 mL), acidified to pH 6 with HCl (1 N), and extracted with ethyl acetate (3×5 mL). The organic layers were combined, dried over anhydrous Na₂SO₄, and filtered. After filtration, the filtrate was concentrated under reduced pressure to afford 4-[1-(tert-butoxycarbonyl)-1,7-diazaspiro[3.5]nonan-7-yl]-2-methylindazole-7-carboxylic acid (160 mg, 92%) as a solid. LCMS (ES, m/z): 401 [M+H]⁺.

### Synthesis of Intermediate C93

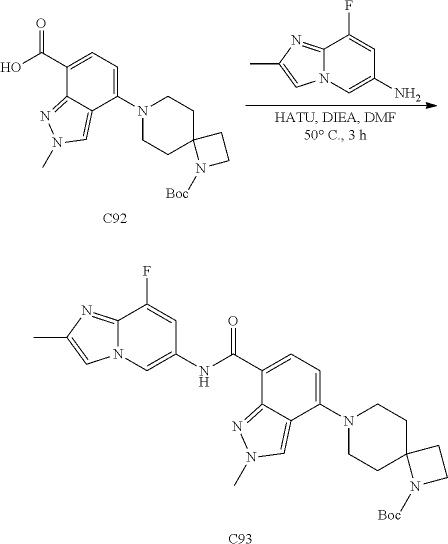 [SMILES: Cn1cc2c(N3CCC4(CC3)CCN4C(=O)OC(C)(C)C)ccc(C(=O)O)c2n1 ; Cc1cn2cc(N)cc(F)c2n1 ; "C92" ; Cc1cn2cc(NC(=O)c3ccc(N4CCC5(CC4)CCN5C(=O)OC(C)(C)C)c4cn(C)nc34)cc(F)c2n1 ; "C93"]

To a stirred solution of 4-[1-(tert-butoxycarbonyl)-1,7-diazaspiro[3.5]nonan-7-yl]-2-methylindazole-7-carboxylic acid (160.0 mg, 0.400 mmol, 1.0 equiv) in DMF (3.2 mL) was added DIEA (206.5 mg, 1.600 mmol, 4.0 equiv), HATU (227.8 mg, 0.600 mmol, 1.5 equiv), and 8-fluoro-2-methylimidazo[1,2-a]pyridin-6-amine (66.0 mg, 0.400 mmol, 1.0 equiv) in portions at room temperature. The resulting mixture was stirred for 3 h at 50° C., then diluted with water (10 mL) and extracted with EtOAc (3×10 mL). The organic layers were combined, washed with water (3×5 mL), dried over anhydrous Na₂SO₄, and filtered. After filtration, the filtrate was concentrated under reduced pressure to give a residue. The residue was purified by silica gel column chromatography, eluted with ethyl acetate to afford tert-butyl 7-[7-({8-fluoro-2-methylimidazo[1,2-a]pyridin-6-yl}carbamoyl)-2-methylindazol-4-yl]-1,7-diazaspiro[3.5]nonane-1-carboxylate (140 mg, 64%) as a solid. LCMS (ES, m/z): 548 [M+H]⁺.

### Synthesis of Compound 301

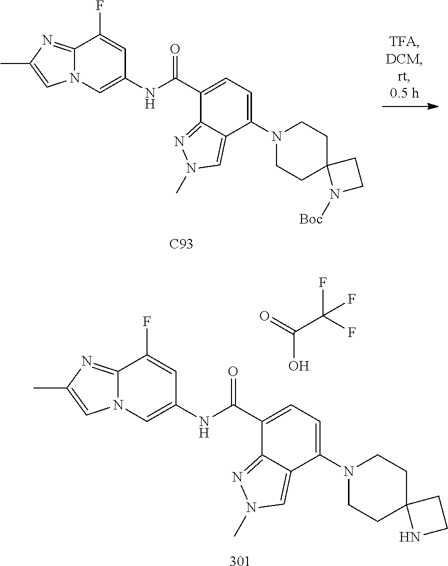

To a stirred solution of tert-butyl 7-[7-({8-fluoro-2-methylimidazo[1,2-a]pyridin-6-yl}carbamoyl)-2-methylindazol-4-yl]-1,7-diazaspiro[3.5]nonane-1-carboxylate (140.0 mg, 0.205 mmol, 1.0 equiv) in DCM (3 mL) was added TFA (1 mL) dropwise at 0° C. The resulting mixture was stirred for 1 h at 0° C., then concentrated under reduced pressure to give a residue. The residue was purified by Prep-HPLC (Condition 3, Gradient 2) to afford 4-{1,7-diazaspiro[3.5]nonan-7-yl}-N-{8-fluoro-2-methylimidazo[1,2-a]pyridin-6-yl}-2-methylindazole-7-carboxamide trifluoroacetic acid salt (11.9 mg, 13%) as a solid. LCMS (ES, m/z): 448 [M+H]⁺. ¹H NMR (300 MHz, DMSO-d₆) δ 11.22 (s, 1H), 9.42 (d, J=1.5 Hz, 1H), 8.86-8.85 (m, 3H), 8.10 (d, J=2.7 Hz, 1H), 8.00 (d, J=8.1 Hz, 1H), 7.75 (d, J=12.0 Hz, 1H), 6.57 (d, J=8.2 Hz, 1H), 4.32 (s, 3H), 3.93 (t, J=7.3 Hz, 2H), 3.65-3.54 (m, 2H), 3.38 (dd, J=13.5, 6.9 Hz, 2H), 2.42-2.35 (m, 5H), 2.19-2.18 (m, 4H).

### Example 83: Synthesis of Compound 337

### Synthesis of Intermediate C94

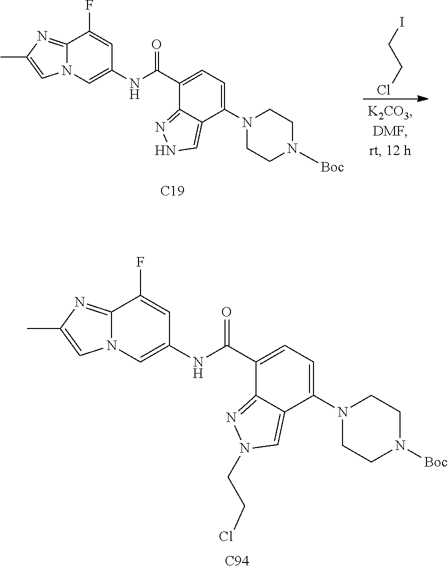

A solution of tert-butyl 4-[7-({8-fluoro-2-methylimidazo[1,2-a]pyridin-6-yl}carbamoyl)-2H-indazol-4-yl]piperazine-1-carboxylate (150 mg, 0.304 mmol, 1 equiv), 1-chloro-2-iodoethane (69.44 mg, 0.365 mmol, 1.2 equiv), and K₂CO₃ (63.01 mg, 0.456 mmol, 1.5 equiv) in DMF (2 mL) was stirred for 12 h at room temperature. The solids were removed by filtration. The filtrate was purified by reverse flash chromatography (Condition 2, Gradient 2) to afford tert-butyl 4-[2-(2-chloroethyl)-7-({8-fluoro-2-methylimidazo[1,2-a]pyridin-6-yl}carbamoyl)indazol-4-yl]piperazine-1-carboxylate (60 mg, 36%) as a solid. LCMS (ES, m/z): 556 [M+H]⁺.

### Synthesis of Compound 337

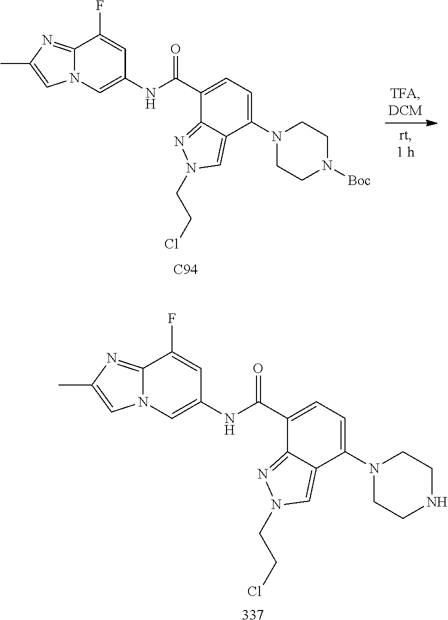

A mixture of tert-butyl 4-[2-(2-chloroethyl)-7-({8-fluoro-2-methylimidazo[1,2-a]pyridin-6-yl}carbamoyl)indazol-4-yl]piperazine-1-carboxylate (80 mg, 0.144 mmol, 1 equiv) and TFA (0.2 mL) in DCM (2 mL) was stirred for 1 h at room temperature. The resulting mixture was basified to pH 8 with 7 N NH₃ (gas) in methanol, then concentrated under reduced pressure to give a residue. The residue was purified by reverse flash (Condition 1, Gradient 2) to afford 2-(2-chloroethyl)-N-{8-fluoro-2-methylimidazo[1,2-a]pyridin-6-yl}-4-(piperazin-1-yl)indazole-7-carboxamide (18 mg, 27%) as a solid. LCMS (ES, m/z): 456 [M+H]⁺. ¹H NMR (300 MHz, DMSO-d₆) δ 11.01 (s, 1H), 9.25 (d, J=1.7 Hz, 1H), 8.92 (s, 1H), 8.01 (d, J=8.1 Hz, 1H), 7.91 (dd, J=3.2, 1.0 Hz, 1H), 7.39 (dd, J=12.5, 1.7 Hz, 1H), 6.51 (d, J=8.2 Hz, 1H), 4.91 (t, J=5.7 Hz, 2H), 4.33 (t, J=5.7 Hz, 2H), 3.45-3.31 (m, 4H), 2.94 (t, J=5.0 Hz, 4H), 2.35 (d, J=0.8 Hz, 3H).

### Example 84: Synthesis of Compound 338

### Synthesis of Intermediate C95

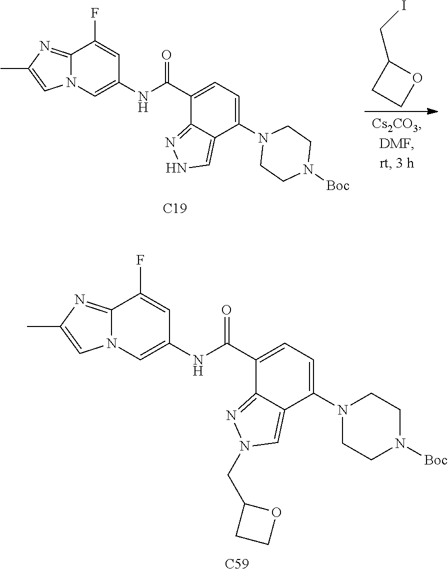 [SMILES: Cc1cn2cc(NC(=O)c3ccc(N4CCN(C(=O)OC(C)(C)C)CC4)c4c[nH]nc34)cc(F)c2n1 ; ICC1CCO1 ; "C19" ; Cc1cn2cc(NC(=O)c3ccc(N4CCN(C(=O)OC(C)(C)C)CC4)c4cn(CC5CCO5)nc34)cc(F)c2n1 ; "C59"]

A mixture of tert-butyl 4-[7-({8-fluoro-2-methylimidazo[1,2-a]pyridin-6-yl}carbamoyl)-2H-indazol-4-yl]piperazine-1-carboxylate (100 mg, 0.203 mmol, 1 equiv), 2-(iodomethyl)oxetane (48.14 mg, 0.244 mmol, 1.2 equiv), and Cs₂CO₃ (198.05 mg, 0.609 mmol, 3.0 equiv) in DMF (2 mL) was stirred for 3 h at room temperature. The reaction mixture was filtered to remove solids. The filtrate was purified by reverse flash chromatography (Condition 2, Gradient 2) to afford tert-butyl 4-[7-({8-fluoro-2-methylimidazo[1,2-a]pyridin-6-yl}carbamoyl)-2-(oxetan-2-ylmethyl)indazol-4-yl]piperazine-1-carboxylate (70 mg, 61%) as a solid. LCMS (ES, m/z): 564 [M+H]⁺.

### Synthesis of Compound 338

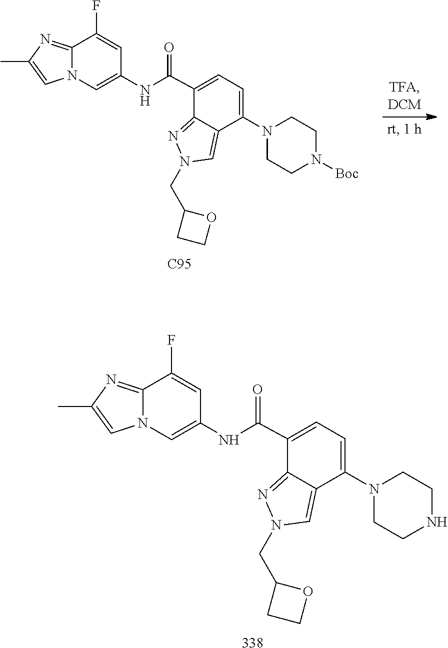

A mixture of tert-butyl 4-[7-({8-fluoro-2-methylimidazo[1,2-a]pyridin-6-yl}carbamoyl)-2-(oxetan-2-ylmethyl)indazol-4-yl]piperazine-1-carboxylate (60 mg, 0.106 mmol, 1 equiv) and TFA (97.10 mg, 0.848 mmol, 8 equiv) in DCM (2 mL) was stirred for 1 h at room temperature. The resulting mixture was basified to pH 8 with 7 N NH₃ (gas) in methanol, then concentrated under reduced pressure to give a residue. The residue was purified by reverse flash chromatography (Condition 1, Gradient 3) to afford N-{8-fluoro-2-methylimidazo[1,2-a]pyridin-6-yl}-2-(oxetan-2-ylmethyl)-4-(piperazin-1-yl)indazole-7-carboxamide (18 mg, 36%) as a solid. LCMS (ES, m/z): 464 [M+H]⁺. ¹H NMR (300 MHz, DMSO-d₆) δ 11.14 (s, 1H), 9.25 (d, J=1.6 Hz, 1H), 8.81 (s, 1H), 8.00 (d, J=8.1 Hz, 1H), 7.95-7.88 (m, 1H), 7.31 (dd, J=12.4, 1.7 Hz, 1H), 6.51 (d, J=8.2 Hz, 1H), 5.29 (t, J=6.0 Hz, 1H), 4.88 (dd, J=13.9, 6.6 Hz, 1H), 4.79 (dd, J=13.8, 4.3 Hz, 1H), 4.64-4.51 (m, 1H), 4.43 (dt, J=9.0, 6.0 Hz, 1H), 3.37 (d, J=5.0 Hz, 4H), 2.94 (s, 4H), 2.78 (dd, J=11.4, 7.4 Hz, 1H), 2.56 (dd, J=9.2, 2.3 Hz, 1H), 2.35 (d, J=0.8 Hz, 3H).

### Example 85: Synthesis of Compound 306

### Synthesis of Intermediate C96

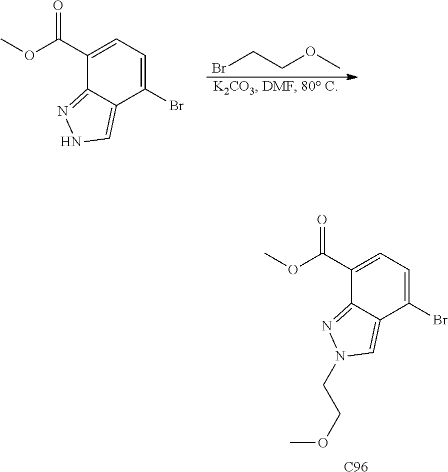

To a stirred mixture of methyl 4-bromo-2H-indazole-7-carboxylate (100 mg, 0.392 mmol, 1 equiv) and 2-bromoethyl methyl ether (81.74 mg, 0.588 mmol, 1.5 equiv) in DMF (2.5 mL) was added K₂CO₃ (108.37 mg, 0.784 mmol, 2 equiv) at room temperature. The resulting mixture was stirred for 5 h at 80° C., then cooled to room temperature. The resulting mixture was quenched with water (5 mL) and extracted with ethyl acetate (2×3 mL). The organic layers were combined, washed with brine (2×2 mL), dried over anhydrous Na₂SO₄, and filtered. After filtration, the filtrate was concentrated under reduced pressure to give a residue. The residue was purified by silica gel column chromatography, eluted with PE/EA (1:1) to afford methyl 4-bromo-2-(2-methoxyethyl)indazole-7-carboxylate (93.5 mg, 76%) as a solid. LCMS (ES, m/z): 313 [M+H]⁺.

### Synthesis of Intermediate C97

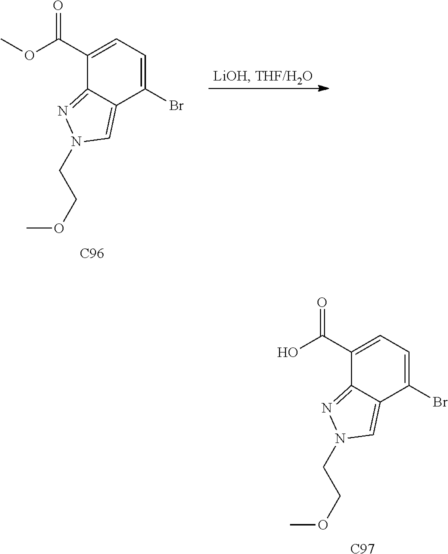 [SMILES: COCCn1cc2c(Br)ccc(C(=O)OC)c2n1 ; "LiOH, THF/H2O" ; "C96" ; COCCn1cc2c(Br)ccc(C(=O)O)c2n1 ; "C97"]

A mixture of methyl 4-bromo-2-(2-methoxyethyl) indazole-7-carboxylate (20 mg, 0.064 mmol, 1 equiv) and LiOH (5 mg, 0.192 mmol, 3 equiv) in water (0.25 mL), THF (0.5 mL) and methanol (0.5 mL) was stirred for 3 h at room temperature. The resulting mixture was concentrated under vacuum to give a residue. The residue was acidified to pH 3 with 1 N HCl. A solid precipitated that was collected by filtration, then washed with water (0.25 mL) to afford 4-bromo-2-(2-methoxyethyl) indazole-7-carboxylic acid (10 mg, 52%) as a solid. LCMS (ES, m/z): 299 [M+H]⁺.

### Synthesis of Intermediate C98

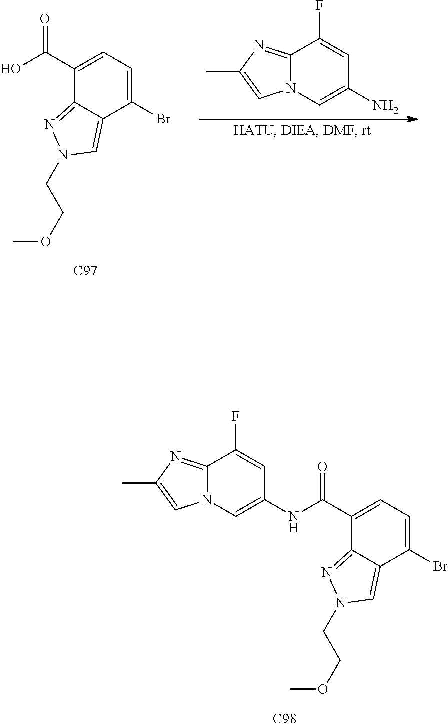

To a stirred of mixture of 4-bromo-2-(2-methoxyethyl) indazole-7-carboxylic acid (550 mg, 1.839 mmol, 1 equiv), DIEA (712 mg, 5.517 mmol, 3 equiv), and HATU (839 mg, 2.207 mmol, 1.2 equiv) in DMF (15 mL) was added 8-fluoro-2-methylimidazo[1,2-a]pyridin-6-amine (364 mg, 2.207 mmol, 1.2 equiv) at room temperature. The resulting mixture was stirred for 3 h at room temperature, then diluted with water (10 mL) and extracted with ethyl acetate (2×20 mL). The organic layers were combined, washed with brine (1×20 mL), dried over anhydrous Na₂SO₄, and filtered. After filtration, the filtrate was concentrated under reduced pressure to give a residue. The residue was purified by silica gel column chromatography, eluted with PE/EA (1:1) to afford 4-bromo-N-{8-fluoro-2-methylimidazo[1,2-a]pyridin-6-yl}-2-(2-methoxyethyl) indazole-7-carboxamide (350 mg, 43%) as a solid. LCMS (ES, m/z): 446 [M+H]⁺.

### Synthesis of Intermediate C99

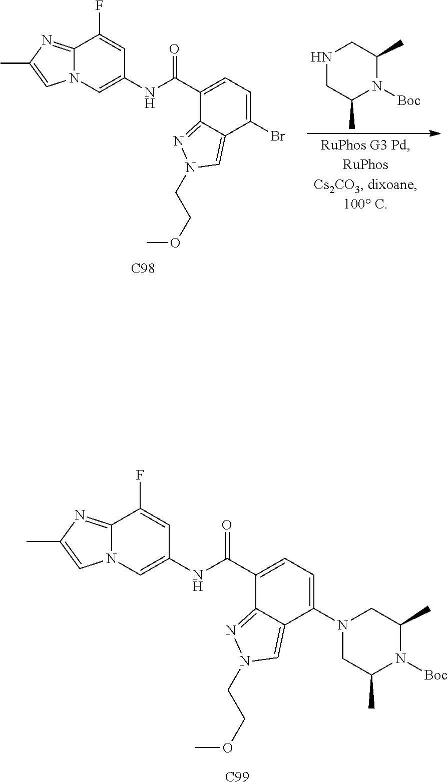

To a stirred mixture of 4-bromo-N-{8-fluoro-2-methylimidazo[1,2-a]pyridin-6-yl}-2-(2-methoxyethyl) indazole-7-carboxamide (100 mg, 0.224 mmol, 1 equiv), rac-tert-butyl (2R,6S)-2,6-dimethylpiperazine-1-carboxylate (96 mg, 0.448 mmol, 2 equiv), and Cs₂CO₃ (219 mg, 0.672 mmol, 3 equiv) in dioxane (5 mL) as added RuPhos (20 mg, 0.045 mmol, 0.2 equiv) and RuPhos Pd G3 (18 mg, 0.022 mmol, 0.1 equiv) at room temperature under nitrogen atmosphere. The resulting mixture was stirred for 3 h at 80° C. under nitrogen atmosphere, then quenched with water (10 mL) and extracted with ethyl acetate (2×10 mL). The organic layers were combined, washed with water (2×5 mL), dried over anhydrous Na₂SO₄, and filtered. After filtration, the filtrate was concentrated under reduced pressure to give a residue. The residue was purified by silica gel column chromatography, eluted with PE/EA (1:2) to afford rac-tert-butyl (2R,6S)-4-[7-({8-fluoro-2-methylimidazo[1,2-a]pyridin-6-yl}carbamoyl)-2-(2-methoxyethyl) indazol-4-yl]-2,6-dimethylpiperazine-1-carboxylate (60 mg, 46%) as a solid. LCMS (ES, m/z): 580 [M+H]⁺.

### Synthesis of Compound 306

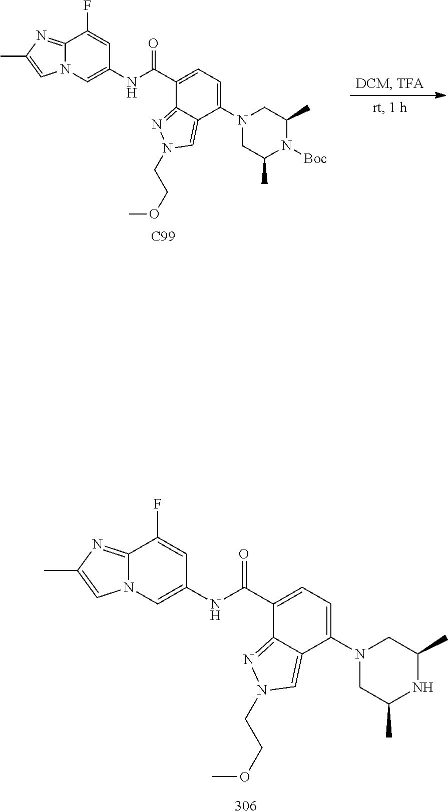

A mixture of tert-butyl (2R,6S)-4-[7-({8-fluoro-2-methylimidazo[1,2-a]pyridin-6-yl}carbamoyl)-2-(2-methoxyethyl) indazol-4-yl]-2,6-dimethylpiperazine-1-carboxylate (50 mg, 0.086 mmol, 1 equiv) and TFA (1 mL) in DCM (2 mL) was stirred for 1 h at room temperature. The resulting mixture was concentrated under vacuum to give a residue. The residue was adjusted to pH 8 with ammonia and purified by Prep-HPLC (Condition 5, Gradient 5) to afford 4-[(3R,5S)-3,5-dimethylpiperazin-1-yl]-N-{8-fluoro-2-methylimidazo[1,2-a]pyridin-6-yl}-2-(2-methoxyethyl) indazole-7-carboxamide (12 mg, 24%) as a solid. LCMS (ES, m/z): 479.56 [M+H]⁺. ¹H NMR (300 MHz, DMSO-d₆) δ 11.12 (s, 1H), 9.23 (d, J=1.6 Hz, 1H), 8.81 (s, 1H), 7.98 (d, J=8.1 Hz, 1H), 7.91 (d, J=3.4 Hz, 1H), 7.31 (dd, J=12.4, 1.7 Hz, 1H), 6.49 (d, J=8.2 Hz, 1H), 4.74 (t, J=5.2 Hz, 2H), 3.95 (t, J=5.2 Hz, 2H), 3.83-3.73 (m, 2H), 3.30 (s, 3H), 2.94 (d, J=8.0 Hz, 2H), 2.45 (d, J=11.0 Hz, 1H), 2.35 (s, 3H), 2.29 (s, 1H), 1.07 (d, J=6.2 Hz, 6H). ¹⁹F NMR (282.3 MHz, DMSO-d₆) δ −132.011.

### Example 86: Synthesis of Compound 294

### Synthesis of Intermediate C100

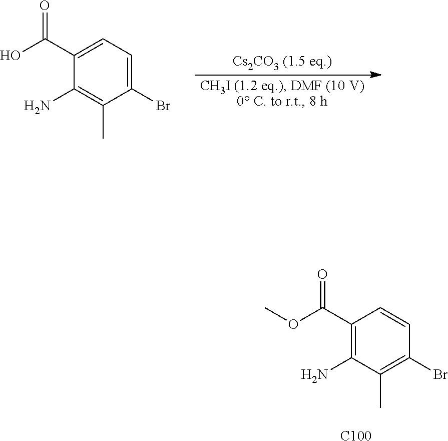

To a stirred mixture of 2-amino-4-bromo-3-methylbenzoic acid (5.00 g, 21.73 mmol, 1.00 equiv) and Cs₂CO₃ (10.62 g, 32.6 mmol, 1.50 equiv) in DMF (50 mL) was added methyl iodide (3.70 g, 26.08 mmol, 1.20 equiv) dropwise at 0° C. under nitrogen atmosphere. The resulting mixture was stirred for 8 h at room temperature under nitrogen atmosphere, then diluted with water and extracted with ethyl acetate (3×200 mL). The organic layers were combined, washed with half saturated aqueous NaCl (3×100 mL), followed by saturated aqueous NaCl (1×100 mL), dried over anhydrous Na₂SO₄, and filtered. After filtration, the filtrate was concentrated under reduced pressure to afford methyl 2-amino-4-bromo-3-methylbenzoate (5 g, 94%) as a solid. LCMS (ES, m/z): 244 [M+H]⁺. ¹H NMR (400 MHz, DMSO-d₆) δ 7.57-7.50 (m, 1H), 6.86-6.78 (m, 3H), 3.80 (s, 3H), 2.24 (s, 3H).

### Synthesis of Intermediate C101

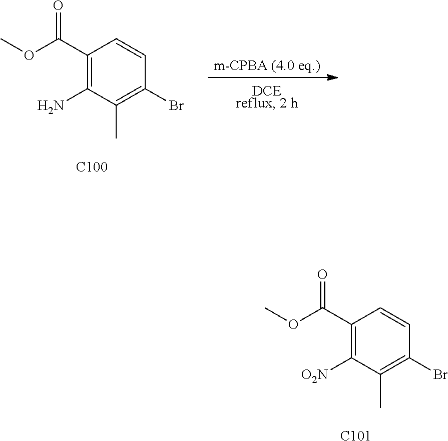

Methyl 2-amino-4-bromo-3-methylbenzoate (3.60 g, 14.75 mmol, 1.00 equiv), m-CPBA (10.18 g, 59.00 mmol, 4.00 equiv) and DCE (80 mL) were combined at room temperature. The resulting mixture was stirred for 2 h at 90° C. under nitrogen atmosphere, then quenched with water at 0° C., and purified by silica gel column chromatography, eluted with PE/EA (5:1) to afford methyl 4-bromo-3-methyl-2-nitrobenzoate (3.5 g, 87%) as a solid.

### Synthesis of Intermediate C102

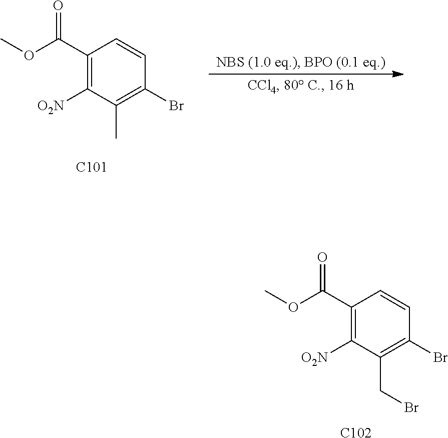 [SMILES: COC(=O)c1ccc(Br)c(C)c1[N+](=O)[O-] ; "CCl4, 80° C., 16 h" ; "NBS (1.0 eq.), BPO (0.1 eq.)" ; "C101" ; COC(=O)c1ccc(Br)c(CBr)c1[N+](=O)[O-] ; "C102"]

Methyl 4-bromo-3-methyl-2-nitrobenzoate (3.50 g, 12.77 mmol, 1.00 equiv), NBS (2.27 g, 12.77 mmol, 1.00 equiv), BPO (0.33 g, 1.28 mmol, 0.10 equiv), and CCl₄ (70 mL) were combined at room temperature. The resulting mixture was stirred for 16 h at 80° C. under nitrogen atmosphere, then purified by silica gel column chromatography, eluted with PE/EA (5:1) to afford 3 g methyl 4-bromo-3-(bromomethyl)-2-nitrobenzoate.

### Synthesis of Intermediate C103

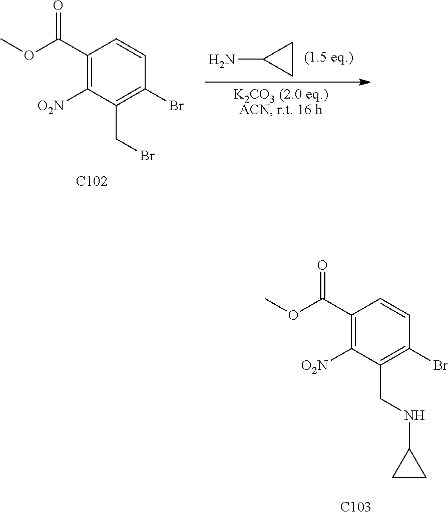

Methyl 4-bromo-3-(bromomethyl)-2-nitrobenzoate (3.00 g, 8.5 mmol, 1.00 equiv), aminocyclopropane (0.73 g, 12.75 mmol, 1.50 equiv), K₂CO₃ (2.35 g, 16.99 mmol, 2.00 equiv), and acetonitrile (60 mL) were combined at room temperature. The resulting mixture was stirred for 16 h at room temperature under nitrogen atmosphere, then purified by Chiral-Prep-HPLC (Condition 2, Gradient 1) to afford methyl 4-bromo-3-[(cyclopropylamino)methyl]-2-nitrobenzoate (400 mg, 14%) as a solid. LCMS (ES, m/z): 329 [M+H]⁺.

### Synthesis of Intermediate C104

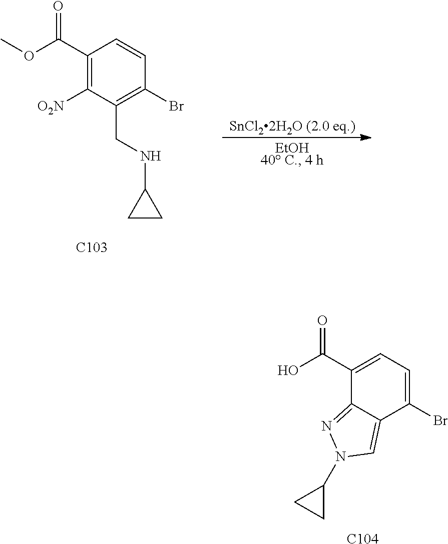 [SMILES: COC(=O)c1ccc(Br)c(CNC2CC2)c1[N+](=O)[O-] ; "EtOH40° C., 4 h" ; "SnCl2·2H2O (2.0 eq.)" ; "C103" ; O=C(O)c1ccc(Br)c2cn(C3CC3)nc12 ; "C104"]

Methyl 4-bromo-3-[(cyclopropylamino)methyl]-2-nitrobenzoate (300 mg, 0.91 mmol, 1.00 equiv), SnCl₂·2H₂O (411.3 mg, 1.82 mmol, 2.00 equiv), and ethanol (10 mL) were combined at room temperature. The resulting mixture was stirred for 4 h at 40° C. under nitrogen atmosphere, then purified by reverse flash chromatography (Condition 4, Gradient 1) to afford 4-bromo-2-cyclopropylindazole-7-carboxylic acid (160 mg, 62%) as a solid. LCMS (ES, m/z): 281 [M+H]⁺.

### Synthesis of Intermediate C105

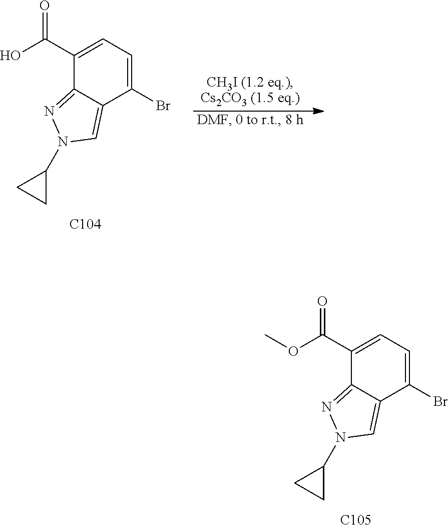 [SMILES: O=C(O)c1ccc(Br)c2cn(C3CC3)nc12 ; "CH3I (1.2 eq.),Cs2CO3 (1.5 eq.)" ; "DMF, 0 to r.t., 8 h" ; "C104" ; COC(=O)c1ccc(Br)c2cn(C3CC3)nc12 ; "C105"]

To a stirred solution of 4-bromo-2-cyclopropylindazole-7-carboxylic acid (150 mg, 0.53 mmol, 1.00 equiv) and Cs₂CO₃ (260.8 mg, 0.8 mmol, 1.50 equiv) in DMF (2 mL) was added CH₃I (90.89 mg, 0.64 mmol, 1.20 equiv) dropwise at 0° C. under nitrogen atmosphere. The resulting mixture was stirred for 8 h at room temperature under nitrogen atmosphere. The resulting mixture was extracted with EtOAc (3×40 mL). The combined organic layers were washed with half saturated aqueous NaCl (3×20 mL) and 1×50 ml of saturated aqueous NaCl, dried over anhydrous Na₂SO₄. After filtration, the filtrate was concentrated under reduced pressure. This resulted in methyl 4-bromo-2-cyclopropylindazole-7-carboxylate (150 mg, 95%) as a solid. LCMS (ES, m/z): 295 [M+H]⁺.

### Synthesis of Intermediate C106

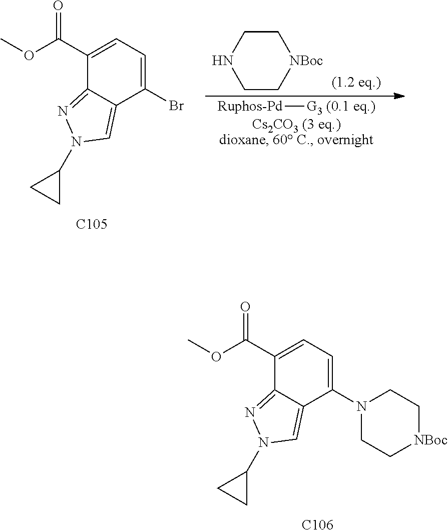

Methyl 4-bromo-2-cyclopropylindazole-7-carboxylate (140 mg, 0.47 mmol, 1.00 equiv), tert-butyl piperazine-1-carboxylate (106.02 mg, 0.57 mmol, 1.20 equiv), Cs₂CO₃ (463.67 mg, 1.42 mmol, 3.00 equiv), RuPhos Palladacycle Gen.3 (39.67 mg, 0.05 mmol, 0.10 equiv), and dioxane (2 mL) were combined at room temperature. The resulting mixture was stirred for 1 overnight at 60° C. under nitrogen atmosphere, then purified by silica gel column chromatography, eluted with PE/EA (5:1) to afford methyl 4-[4-(tert-butoxycarbonyl) piperazin-1-yl]-2-cyclopropylindazole-7-carboxylate (87 mg, 46%) as a solid. LCMS (ES, m/z): 401 [M+H]⁺. 1H NMR (400 MHz, DMSO-d₆) δ 8.64 (s, 1H), 7.84 (d, J=8.1 Hz, 1H), 6.36 (d, J=8.2 Hz, 1H), 4.09 (tt, J=7.6, 3.9 Hz, 1H), 3.80 (s, 3H), 3.57-3.50 (m, 4H), 3.36 (dd, J=6.4, 3.8 Hz, 4H), 1.44 (s, 9H), 1.31 (td, J=4.7, 4.3, 3.1 Hz, 2H), 1.19-1.07 (m, 2H).

### Synthesis of Intermediate C107

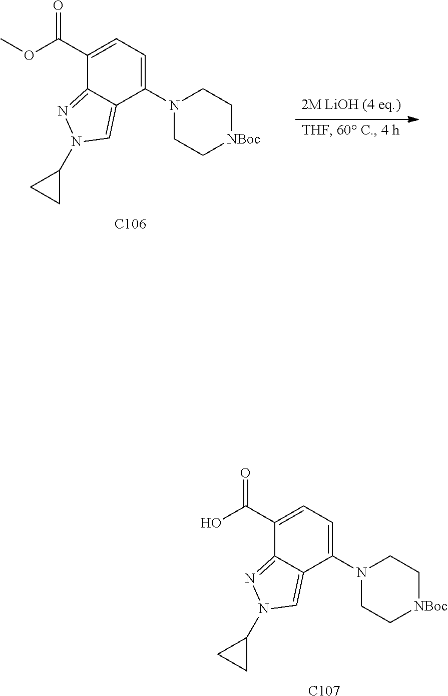 [SMILES: COC(=O)c1ccc(N2CCN(C(=O)OC(C)(C)C)CC2)c2cn(C3CC3)nc12 ; "2M LiOH (4 eq.)" ; "THF, 60° C., 4 h" ; "C106" ; CC(C)(C)OC(=O)N1CCN(c2ccc(C(=O)O)c3nn(C4CC4)cc23)CC1 ; "C107"]

Methyl 4-[4-(tert-butoxycarbonyl) piperazin-1-yl]-2-cyclopropylindazole-7-carboxylate (60 mg, 0.15 mmol, 1.00 equiv), LiOH (14.35 mg, 0.6 mmol, 4.00 equiv) and THF (2 mL) were combined at room temperature. The resulting mixture was stirred for 4 h at 60° C. under nitrogen atmosphere, then acidified to pH 6 with HCl (aq.) and extracted with ethyl acetate (3×20 mL). The organic layers were combined, washed with water (3×20 mL), dried over anhydrous Na₂SO₄, and filtered. After filtration, the filtrate was concentrated under reduced pressure to afford 4-[4-(tert-butoxycarbonyl) piperazin-1-yl]-2-cyclopropylindazole-7-carboxylic acid (54 mg, 93%) as a solid. LCMS (ES, m/z): 387 [M+H]⁺.

### Synthesis of Intermediate C108

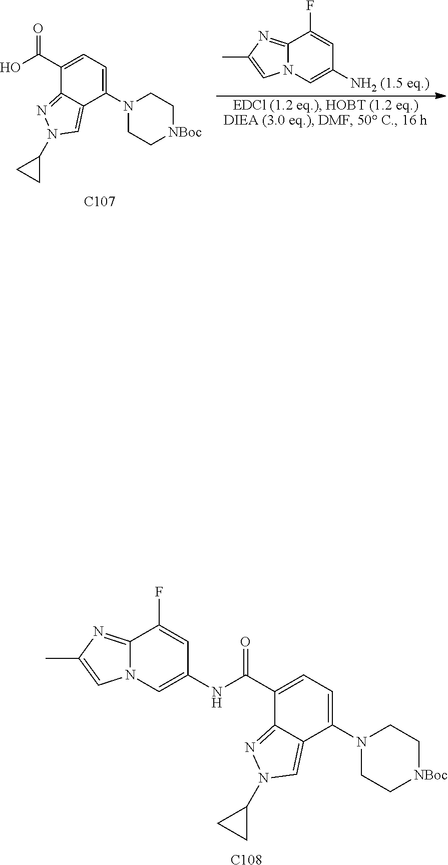

A mixture of 4-[4-(tert-butoxycarbonyl) piperazin-1-yl]-2-cyclopropylindazole-7-carboxylic acid (30 mg, 0.08 mmol, 1.00 equiv), 8-fluoro-2-methylimidazo[1,2-a] pyridin-6-amine (19.23 mg, 0.12 mmol, 1.50 equiv), EDCI (17.86 mg, 0.09 mmol, 1.20 equiv), HOBT (12.59 mg, 0.09 mmol, 1.20 equiv), DIEA (30.1 mg, 0.23 mmol, 3.00 equiv), and DMF (1 mL) was stirred for 16 h at 50° C. under nitrogen atmosphere. The resulting mixture was extracted with ethyl acetate (3×20 mL). The organic layers were combined, washed with water (3×20 mL), dried over anhydrous Na₂SO₄, and filtered. After filtration, the filtrate was concentrated under reduced pressure to afford tert-butyl 4-[2-cyclopropyl-7-({8-fluoro-2-methylimidazo[1,2-a] pyridin-6-yl}carbamoyl) indazol-4-yl] piperazine-1-carboxylate (35 mg, 84%) as a solid. LCMS (ES, m/z): 534 [M+H]⁺.

### Synthesis of Compound 294

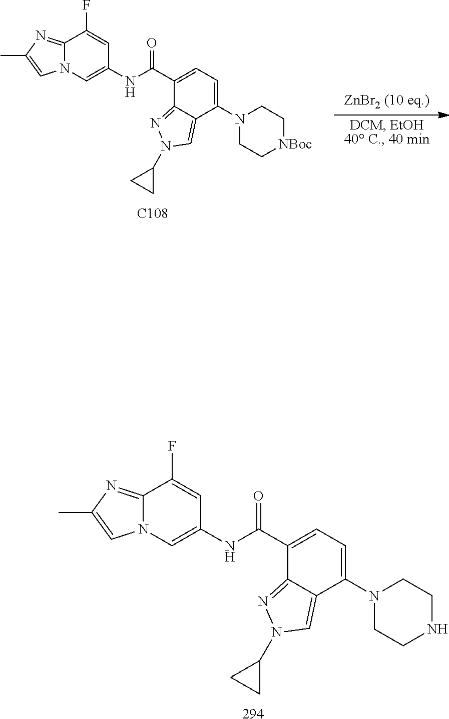

A mixture of tert-butyl 4-[2-cyclopropyl-7-({8-fluoro-2-methylimidazo[1,2-a] pyridin-6-yl}carbamoyl) indazol-4-yl]piperazine-1-carboxylate (33 mg, 0.06 mmol, 1.00 equiv) and ZnBr₂ (139.28 mg, 0.62 mmol, 10.00 equiv) in DCM (0.5 mL) was stirred for 30 min at 40° C. under nitrogen atmosphere. To the reaction mixture was added ethanol (0.5 mL), and the resulting mixture was stirred for an additional 10 min. The resulting product was purified by Prep-HPLC (Condition 6, Gradient 1) to afford 2-cyclopropyl-N-{8-fluoro-2-methylimidazo[1,2-a] pyridin-6-yl}-4-(piperazin-1-yl) indazole-7-carboxamide (16.5 mg, 60.93%) as a solid. LCMS (ES, m/z): 434 [M+H]⁺. ¹H NMR (400 MHz, DMSO-d₆) δ 10.93 (s, 1H), 9.18 (d, J=1.6 Hz, 1H), 8.85 (s, 1H), 7.97 (d, J=8.1 Hz, 1H), 7.91 (d, J=3.1 Hz, 1H), 7.18 (dd, J=12.2, 1.7 Hz, 1H), 6.48 (d, J=8.2 Hz, 1H), 4.28 (tt, J=7.6, 3.9 Hz, 1H), 3.34 (d, J=9.9 Hz, 4H), 2.91 (t, J=5.0 Hz, 4H), 2.35 (d, J=0.9 Hz, 3H), 1.47 (td, J=5.2, 4.4, 3.1 Hz, 2H), 1.25-1.12 (m, 2H).

### Example 87: Synthesis of Compound 275

### Synthesis of Intermediate C109

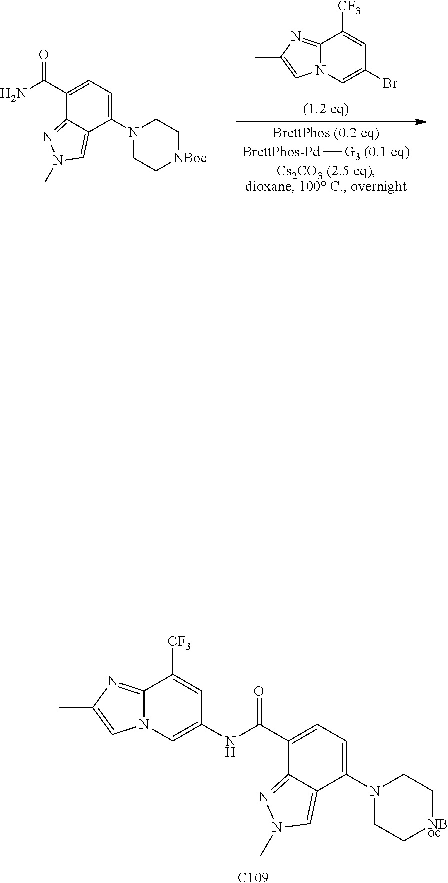

A mixture of tert-butyl 4-(7-carbamoyl-2-methylindazol-4-yl)piperazine-1-carboxylate (100.0 mg, 0.27 mmol, 1.00 equiv), 6-bromo-2-methyl-8-(trifluoromethyl)imidazo[1,2-a]pyridine (93.1 mg, 0.33 mmol, 1.2 equiv), Cs₂CO₃ (226.6 mg, 0.69 mmol, 2.50 equiv), BrettPhos (29.8 mg, 0.05 mmol, 0.2 equiv), BrettPhos-Pd-G₃ (25.2 mg, 0.02 mmol, 0.1 equiv) and dioxane (5 mL) was evacuated and flushed three times with nitrogen. The resulting solution was stirred for 16 h at 100° C., then quenched with water and extracted with ethyl acetate (3×20 mL). The organic layers were combined, washed with saturated aqueous NaCl (1×50 mL), dried over anhydrous sodium sulfate, and filtered. The filtrate was concentrated under vacuum to give a residue. The residue was applied onto a silica gel column with ethyl acetate/petroleum ether to afford tert-butyl 4-(2-methyl-7-{[2-methyl-8-(trifluoromethyl)imidazo[1,2-a]pyridin-6-yl]carbamoyl}indazol-4-yl)piperazine-1-carboxylate (100 mg, 64%) as a solid. LCMS (ES, m/z): 558 [M+H]⁺.

### Synthesis of Compound 275

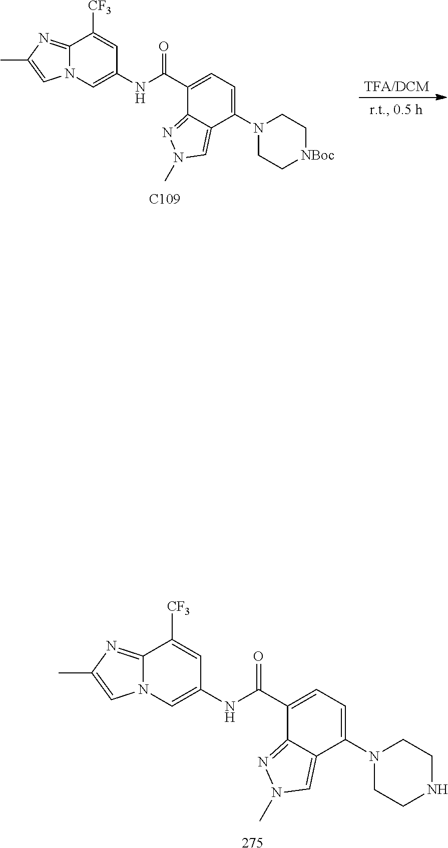

A mixture of tert-butyl 4-(2-methyl-7-{[2-methyl-8-(trifluoromethyl)imidazo[1,2-a]pyridin-6-yl]carbamoyl} indazol-4-yl)piperazine-1-carboxylate (100.0 mg, 0.18 mmol, 1.00 equiv), DCM (2.0 mL), and TFA (0.5 mL) was stirred for 30 min at room temperature. The resulting mixture was concentrated under vacuum to give a residue. The residue was purified by Prep-HPLC (Condition 6, Gradient 2) to afford 2-methyl-N-[2-methyl-8-(trifluoromethyl)imidazo[1,2-a]pyridin-6-yl]-4-(piperazin-1-yl) indazole-7-carboxamide (36.7 mg, 44.2%) as a solid. LCMS (ES, m/z): 458 [M+H]⁺. ¹H-NMR (400 MHz, DMSO-d₆) δ 11.09 (s, 1H), 9.51 (d, J=1.9 Hz, 1H), 8.81 (s, 1H), 8.02-7.94 (m, 2H), 7.83-7.78 (m, 1H), 6.49 (d, J=8.2 Hz, 1H), 4.29 (s, 3H), 3.39-3.32 (m, 4H), 2.95-2.88 (m, 4H), 2.39 (s, 3H).

### Example 88: Synthesis of Compound 307

### Synthesis of Intermediate C110

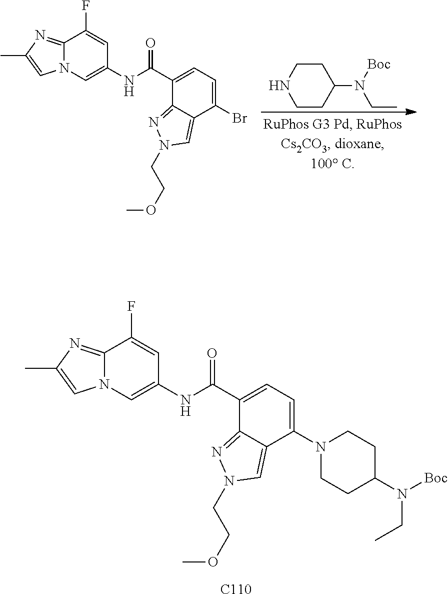 [SMILES: COCCn1cc2c(Br)ccc(C(=O)Nc3cc(F)c4nc(C)cn4c3)c2n1 ; CCN(C(=O)OC(C)(C)C)C1CCNCC1 ; CCN(C(=O)OC(C)(C)C)C1CCN(c2ccc(C(=O)Nc3cc(F)c4nc(C)cn4c3)c3nn(CCOC)cc23)CC1 ; "C110"]

To a stirred mixture of 4-bromo-N-{8-fluoro-2-methylimidazo[1,2-a]pyridin-6-yl}-2-(2-methoxyethyl) indazole-7-carboxamide (100 mg, 0.224 mmol, 1 equiv), tert-butyl N-ethyl-N-(piperidin-4-yl)carbamate (102 mg, 0.448 mmol, 2 equiv), and Cs₂CO₃ (219 mg, 0.672 mmol, 3 equiv) in dioxane (6 mL) was added RuPhos (21 mg, 0.045 mmol, 0.2 equiv) and RuPhos Pd G3 (18 mg, 0.022 mmol, 0.1 equiv) at room temperature under nitrogen atmosphere. The resulting mixture was stirred for 3 h at 80° C. under nitrogen atmosphere, then quenched with water (10 mL) and extracted with ethyl acetate (2×10 mL). The organic layers were combined, washed with brine (2×5 mL), dried over anhydrous Na₂SO₄, and filtered. After filtration, the filtrate was concentrated under reduced pressure to give a residue. The residue was purified by silica gel column chromatography, eluted with PE/EA (3:1) to afford tert-butyl N-ethyl-N-{1-[7-({8-fluoro-2-methylimidazo[1,2-a]pyridin-6-yl}carbamoyl)-2-(2-methoxyethyl) indazol-4-yl]piperidin-4-yl}carbamate (45 mg, 34%) as a solid. LCMS (ES, m/z): 594.0 [M+H]⁺.

### Synthesis of Compound 307

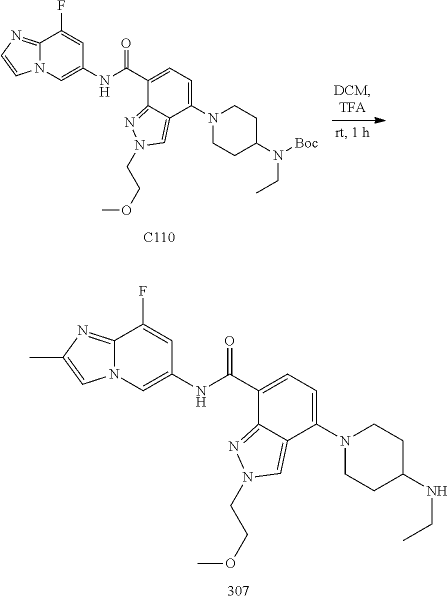 [SMILES: CCN(C(=O)OC(C)(C)C)C1CCN(c2ccc(C(=O)Nc3cc(F)c4nccn4c3)c3nn(CCOC)cc23)CC1 ; "C110" ; CCNC1CCN(c2ccc(C(=O)Nc3cc(F)c4nc(C)cn4c3)c3nn(CCOC)cc23)CC1]

A mixture of tert-butyl N-ethyl-N-{1-[7-({8-fluoro-2-methylimidazo[1,2-a]pyridin-6-yl}carbamoyl)-2-(2-methoxyethyl) indazol-4-yl]piperidin-4-yl}carbamate (50 mg, 0.084 mmol, 1 equiv) and TFA (2 mL) in DCM (4 mL) was stirred for 1 h at room temperature. The resulting mixture was concentrated under reduced pressure to give a residue. The residue was neutralized to pH 8 with 7 N NH₃(g) in methanol, then purified by Prep-HPLC (Condition 2, Gradient 6) to afford 4-[4-(ethylamino)piperidin-1-yl]-N-{8-fluoro-2-methylimidazo[1,2-a]pyridin-6-yl}-2-(2-methoxyethyl) indazole-7-carboxamide (15 mg, 36%) as a solid. LCMS (ES, m/z): 494 [M+H]⁺. 1H NMR (300 MHz, DMSO-d6) δ 11.11 (s, 1H), 9.23 (d, J=1.6 Hz, 1H), 8.77 (s, 1H), 7.98 (d, J=8.1 Hz, 1H), 7.91 (d, J=3.5 Hz, 1H), 7.31 (dd, J=12.4, 1.7 Hz, 1H), 6.49 (d, J=8.2 Hz, 1H), 4.73 (t, J=5.1 Hz, 2H), 3.95 (t, J=5.2 Hz, 2H), 3.91 (s, 2H), 3.86 (s, 1H), 3.30 (s, 3H), 3.07 (t, J=11.5 Hz, 2H), 2.72-2.54 (m, 3H), 2.35 (s, 3H), 1.97 (d, J=12.5 Hz, 2H), 1.61 (s, 1H), 1.44 (q, J=10.1 Hz, 2H), 1.04 (t, J=7.1 Hz, 3H). ¹⁹F NMR (282 MHz, DMSO-d₆) δ −132.01, −132.02.

### Example 89: Synthesis of Compounds 310 and 330

### Synthesis of Intermediate C111

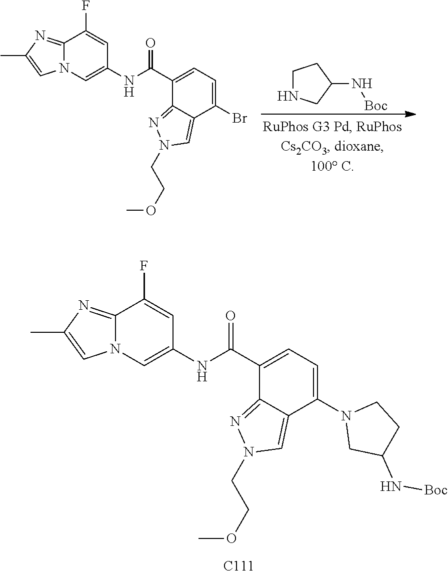 [SMILES: COCCn1cc2c(Br)ccc(C(=O)Nc3cc(F)c4nc(C)cn4c3)c2n1 ; CC(C)(C)OC(=O)NC1CCNC1 ; COCCn1cc2c(N3CCC(NC(=O)OC(C)(C)C)C3)ccc(C(=O)Nc3cc(F)c4nc(C)cn4c3)c2n1 ; "C111"]

To a stirred mixture of 4-bromo-N-{8-fluoro-2-methylimidazo[1,2-a]pyridin-6-yl}-2-(2-methoxyethyl) indazole-7-carboxamide (100 mg, 0.224 mmol, 1 equiv), tert-butyl pyrrolidin-3-ylcarbamate (83 mg, 0.448 mmol, 2 equiv), and Cs₂CO₃ (219 mg, 0.672 mmol, 3 equiv) in 1,4-dioxane (6 mL) was added RuPhos (21 mg, 0.045 mmol, 0.2 equiv) and RuPhos Pd G3 (19 mg, 0.022 mmol, 0.1 equiv) at room temperature under nitrogen atmosphere. The resulting mixture was stirred for 3 h at 80° C. under nitrogen atmosphere, then quenched with water (10 mL) at room temperature and extracted with ethyl acetate (3×10 mL). The organic layers were combined, washed with water (2×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl (1-(7-((8-fluoro-2-methylimidazo[1,2-a]pyridin-6-yl)carbamoyl)-2-(2-methoxyethyl)-2H-indazol-4-yl)pyrrolidin-3-yl)carbamate (50 mg, 40%) as a solid. LCMS (ES, m/z): 552.0 [M+H]⁺.

### Synthesis of Intermediate C112

A mixture of tert-butyl N-{1-[7-({8-fluoro-2-methylimidazo[1,2-a]pyridin-6-yl}carbamoyl)-2-(2-methoxyethyl) indazol-4-yl]pyrrolidin-3-yl}carbamate (100 mg, 0.181 mmol, 1 equiv) and TFA (0.8 mL) in DCM (2 mL) was stirred for 1 h at room temperate. The resulting mixture was concentrated under vacuum to give a residue. The residue was basified to pH 8 with 7 M NH₃(g) in methanol, then purified by reverse flash chromatography (Condition 1, Gradient 4) to afford 4-(3-aminopyrrolidin-1-yl)-N-{8-fluoro-2-methylimidazo[1,2-a]pyridin-6-yl}-2-(2-methoxyethyl) indazole-7-carboxamide (36 mg, 44%) as a solid. LCMS (ES, m/z): 452 [M+H]⁺.

### Synthesis of Compounds 310 and 330

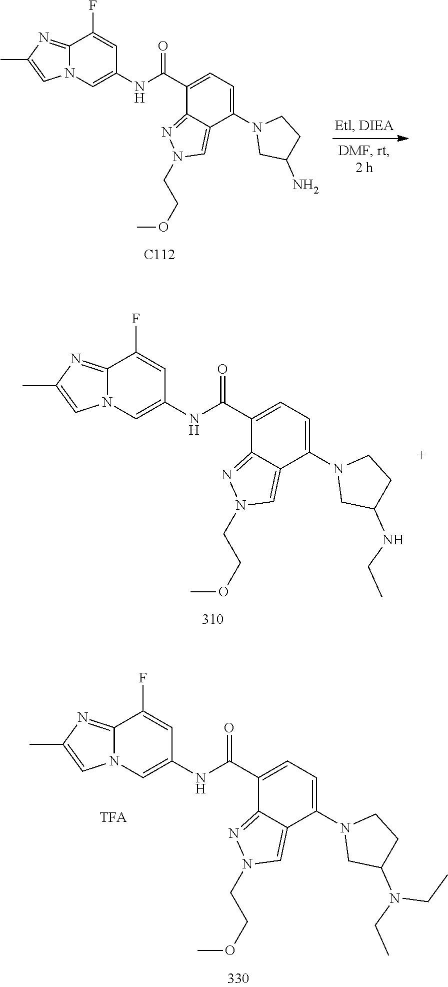 [SMILES: COCCn1cc2c(N3CCC(N)C3)ccc(C(=O)Nc3cc(F)c4nc(C)cn4c3)c2n1 ; "C112" ; CCNC1CCN(c2ccc(C(=O)Nc3cc(F)c4nc(C)cn4c3)c3nn(CCOC)cc23)C1 ; CCN(CC)C1CCN(c2ccc(C(=O)Nc3cc(F)c4nc(C)cn4c3)c3nn(CCOC)cc23)C1]

To a stirred solution of 4-(3-aminopyrrolidin-1-yl)-N-{8-fluoro-2-methylimidazo[1,2-a]pyridin-6-yl}-2-(2-methoxyethyl) indazole-7-carboxamide (100 mg, 0.221 mmol, 1 equiv) and DIEA (129 mg, 0.996 mmol, 4.5 equiv) in DMF (5 mL) was added iodoethane (62 mg, 0.398 mmol, 1.8 equiv) dropwise at 0° C. The resulting mixture was stirred for 2 h at room temperature, then diluted with water (10 mL) and extracted with ethyl acetate (3×10 mL). The organic layers were combined, washed with brine (1×10 mL), dried over anhydrous Na₂SO₄, and filtered. After filtration, the filtrate was concentrated under reduced pressure to give a residue. The residue was purified by Prep-HPLC (Condition 5, Gradient 6) to afford 4-(3-(ethylamino)pyrrolidin-1-yl)-N-(8-fluoro-2-methylimidazo[1,2-a]pyridin-6-yl)-2-(2-methoxyethyl)-2H-indazole-7-carboxamide (6 mg, 5%) and 4-(3-(diethylamino)pyrrolidin-1-yl)-N-(8-fluoro-2-methylimidazo[1,2-a]pyridin-6-yl)-2-(2-methoxyethyl)-2H-indazole-7-carboxamide as solids. 4-(3-(diethylamino)pyrrolidin-1-yl)-N-(8-fluoro-2-methylimidazo[1,2-a]pyridin-6-yl)-2-(2-methoxyethyl)-2H-indazole-7-carboxamide was further purified by prep-HPLC (Condition 7, Gradient 1) to afford 4-(3-(diethylamino)pyrrolidin-1-yl)-N-(8-fluoro-2-methylimidazo[1,2-a]pyridin-6-yl)-2-(2-methoxyethyl)-2H-indazole-7-carboxamide compound with 2,2,2-trifluoro-113-ethan-1-one (5 mg, 4%) as a solid. Compound 310: LCMS (ES, m/z): 480 [M+H]⁺. ¹H NMR (300 MHz, DMSO-d6) δ 11.07 (s, 1H), 9.22 (d, J=1.7 Hz, 1H), 8.84 (s, 1H), 7.96 (d, J=8.2 Hz, 1H), 7.90 (d, J=3.0 Hz, 1H), 7.29 (dd, J=12.4, 1.7 Hz, 1H), 6.06 (d, J=8.4 Hz, 1H), 4.72 (t, J=5.1 Hz, 2H), 3.95 (t, J=5.1 Hz, 2H), 3.82 (d, J=16.1 Hz, 2H), 3.70 (s, 2H), 3.31 (s, 3H), 2.83 (s, 2H), 2.35 (s, 3H), 2.07 (s, 1H), 1.13 (t, J=7.0 Hz, 3H). ¹⁹F NMR (282 MHz, DMSO-d6) δ −132.117. Compound 330: LCMS: (ES, m/z): 508 [M+H]⁺. ¹H NMR (300 MHz, Methanol-d₄) δ 9.46-9.36 (m, 1H), 8.71 (d, J=1.6 Hz, 1H), 8.12-7.93 (m, 3H), 7.85 (d, J=10.5 Hz, 1H), 6.23-6.12 (m, 1H), 4.75 (t, J=5.0 Hz, 2H), 4.29 (t, J=8.2 Hz, 1H), 4.18 (t, J=8.9 Hz, 1H), 4.02 (d, J=12.1 Hz, 1H), 4.02 (s, 3H), 3.95-3.79 (m, 2H), 3.42 (dd, J=11.0, 5.1 Hz, 7H), 2.73-2.63 (m, 1H), 2.53 (d, J=3.2 Hz, 3H), 2.40 (q, J=10.8, 10.2 Hz, 1H), 1.41 (t, J=7.2 Hz, 6H). ¹⁹F NMR (282 MHz, Methanol-d₄) δ −76.86, −133.40-−134.87 (m).

### Example 90: Synthesis of Compound 340

### Synthesis of Intermediate C113

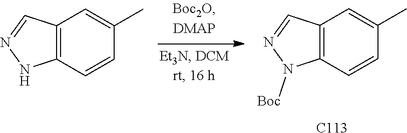

To a stirred solution of 5-methyl-1H-indazole (500 mg, 3.783 mmol, 1 equiv) in DCM (10 mL) was added Et₃N (1.15 g, 11.349 mmol, 3 equiv), Boc₂O (908.2 mg, 4.161 mmol, 1.1 equiv), and DMAP (46.2 mg, 0.378 mmol, 0.1 equiv) in portions at room temperature. The resulting mixture was stirred for 16 h at room temperature, then washed with water (2×10 mL). The organic layer was dried over anhydrous Na₂SO₄ and filtered. After filtration, the filtrate was concentrated under reduced pressure to give a residue. The residue was purified by silica gel column chromatography, eluted with PE/EA (2:1) to afford tert-butyl 5-methylindazole-1-carboxylate (500 mg, 57%) as a solid. LCMS (ES, m/z): 233 [M+H]⁺.

### Synthesis of Intermediate C114

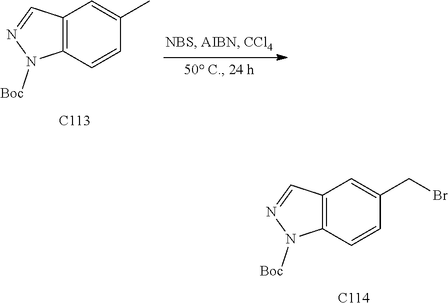 [SMILES: Cc1ccc2c(cnn2C(=O)OC(C)(C)C)c1 ; "C113" ; CC(C)(C)OC(=O)n1ncc2cc(CBr)ccc21 ; "C114"]

To a stirred solution of tert-butyl 5-methylindazole-1-carboxylate (200.0 mg, 0.861 mmol, 1 equiv) in CCl4 (6 mL) was added NBS (183.9 mg, 1.033 mmol, 1.2 equiv) and AIBN (14.1 mg, 0.086 mmol, 0.1 equiv) in portions at room temperature. The resulting mixture was stirred for 24 h at 50° C., then concentrated under reduced pressure to give a residue. The residue was purified by silica gel column chromatography, eluted with PE/EA (1:1) to afford tert-butyl 5-(bromomethyl) indazole-1-carboxylate (65 mg, 24%) as a solid. LCMS (ES, m/z): 311 [M+H]⁺.

### Synthesis of Intermediate C115

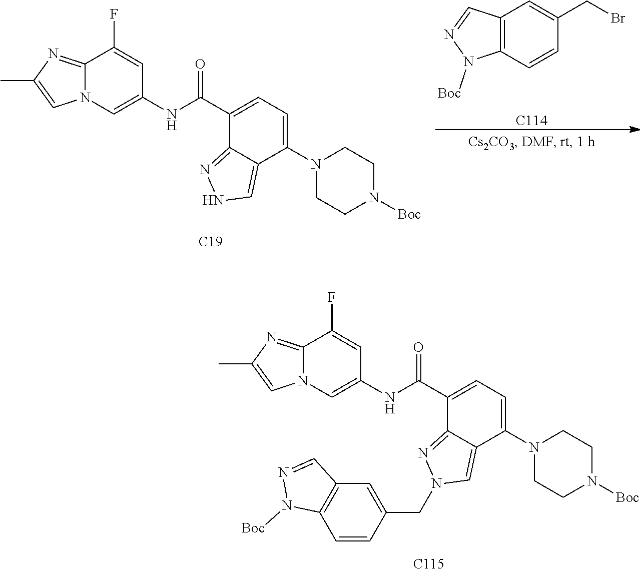 [SMILES: "C114" ; Cc1cn2cc(NC(=O)c3ccc(N4CCN(C(=O)OC(C)(C)C)CC4)c4c[nH]nc34)cc(F)c2n1 ; CC(C)(C)OC(=O)n1ncc2cc(CBr)ccc21 ; "C19" ; Cc1cn2cc(NC(=O)c3ccc(N4CCN(C(=O)OC(C)(C)C)CC4)c4cn(Cc5ccc6c(cnn6C(=O)OC(C)(C)C)c5)nc34)cc(F)c2n1 ; "C115"]

To a stirred solution of tert-butyl 4-[7-({8-fluoro-2-methylimidazo[1,2-a]pyridin-6-yl}carbamoyl)-2H-indazol-4-yl]piperazine-1-carboxylate (70.0 mg, 0.142 mmol, 1.0 equiv) and tert-butyl 5-(bromomethyl) indazole-1-carboxylate (66.2 mg, 0.213 mmol, 1.5 equiv) in DMF (1.7 mL) was added Cs₂CO₃ (138.6 mg, 0.426 mmol, 3 equiv) at room temperature. The resulting mixture was stirred for 1 h at room temperature, then diluted with water (6 mL) and extracted with ethyl acetate (3×6 mL). The organic layers were combined, washed with water (3×10 mL), dried over anhydrous Na₂SO₄, and filtered. After filtration, the filtrate was concentrated under reduced pressure to give a residue. The residue was purified by silica gel column chromatography, eluted with ethyl acetate to afford tert-butyl 5-({4-[4-(tert-butoxycarbonyl)piperazin-1-yl]-7-({8-fluoro-2-methylimidazo[1,2-a]pyridin-6-yl}carbamoyl) indazol-2-yl}methyl) indazole-1-carboxylate (40 mg, 39%) as a solid. LCMS (ES, m/z): 724 [M+H]⁺.

### Synthesis of Compound 340

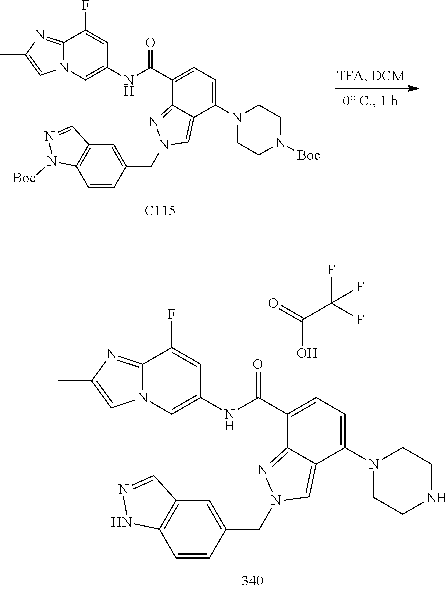

To a stirred solution of tert-butyl 5-({4-[4-(tert-butoxycarbonyl)piperazin-1-yl]-7-({8-fluoro-2-methylimidazo[1,2-a]pyridin-6-yl}carbamoyl) indazol-2-yl}methyl) indazole-1-carboxylate (35.0 mg, 0.048 mmol, 1.0 equiv) in DCM (1 mL) was added TFA (0.3 mL) dropwise at 0° C. The resulting mixture was stirred for 1 h at 0° C., then concentrated under reduced pressure to give a residue. The residue was purified by Prep-HPLC (Condition 3, Gradient 2) to afford N-{8-fluoro-2-methylimidazo[1,2-a]pyridin-6-yl}-2-(1H-indazol-5-ylmethyl)-4-(piperazin-1-yl) indazole-7-carboxamide trifluoroacetate (7.8 mg, 31%) as a solid. LCMS (ES, m/z): 524 [M+H]⁺. ¹H NMR (400 MHz, DMSO-d₆) δ 13.15-13.14 (m, 1H), 11.25 (s, 1H), 9.36 (s, 1H), 9.10 (s, 1H), 8.99-8.98 (m, 2H), 8.10 (s, 1H), 8.08 (d, J=7.6 Hz, 1H), 8.02 (d, J=8.0 Hz, 1H), 7.98 (s, 1H), 7.61-7.51 (m, 2H), 7.48 (d, J=11.9 Hz, 1H), 6.62 (d, J=8.1 Hz, 1H), 5.89 (s, 2H), 3.64-3.57 (m, 4H), 3.36-3.35 (m, 4H), 2.42 (s, 3H).

### Example 91: Synthesis of Compound 314

### Synthesis of Intermediate C116

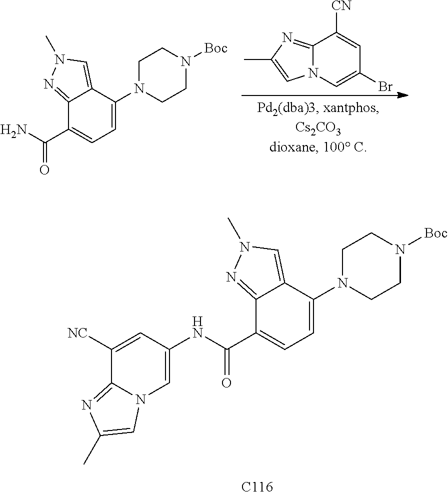

To a mixture of 4-[4-(2,2-dimethylpropanoyl)piperazin-1-yl]-2-methylindazole-7-carboxamide (60 mg, 0.167 mmol, 1 equiv), 6-bromo-2,8-dimethylimidazo[1,2-a]pyrazine (45.34 mg, 0.200 mmol, 1.2 equiv), and Cs₂CO₃ (108.91 mg, 0.334 mmol, 2.0 equiv) in dioxane (2 mL) was added Xantphos (9.67 mg, 0.017 mmol, 0.1 equiv) and Pd₂(dba)₃ (7.65 mg, 0.008 mmol, 0.05 equiv). The reaction mixture was stirred overnight at 100° C. under a nitrogen atmosphere, then concentrated under reduced pressure to give a residue. The residue was purified by silica gel column chromatography, eluted with PE/EA (1:2) to afford N-{2,8-dimethylimidazo[1,2-a]pyrazin-6-yl}-4-[4-(2,2-dimethylpropanoyl)piperazin-1-yl]-2-methylindazole-7-carboxamide (40 mg, 49%) as a solid. LCMS (ES, m/z): 515 [M+H]⁺.

### Synthesis of Compound 314

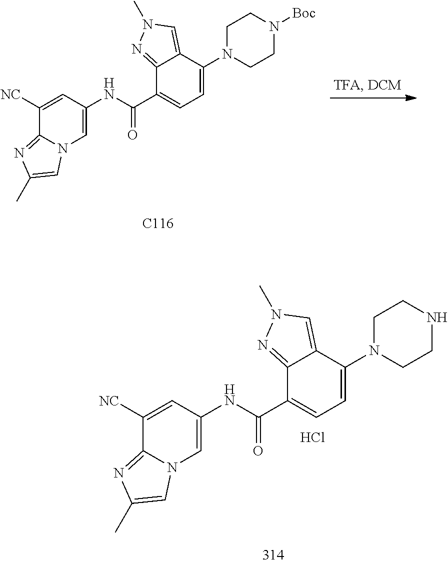 [SMILES: Cc1cn2cc(NC(=O)c3ccc(N4CCN(C(=O)OC(C)(C)C)CC4)c4cn(C)nc34)cc(C#N)c2n1 ; "C116" ; Cc1cn2cc(NC(=O)c3ccc(N4CCNCC4)c4cn(C)nc34)cc(C#N)c2n1 ; Cl]

A solution of tert-butyl 4-[7-({8-cyano-2-methylimidazo[1,2-a]pyridin-6-yl}carbamoyl)-2-methylindazol-4-yl]piperazine-1-carboxylate (40 mg, 0.078 mmol, 1 equiv) in DCM was treated with TFA (0.5 mL, 6.732 mmol, 86.60 equiv). The reaction mixture was stirred for 1 h at room temperature, then concentrated under reduced pressure to give a residue. The residue was purified by Prep-HPLC (Condition 8, Gradient 1) to afford N-{8-cyano-2-methylimidazo[1,2-a]pyridin-6-yl}-2-methyl-4-(piperazin-1-yl) indazole-7-carboxamide hydrochloride (20.7 mg, 58%) as a solid. LCMS (ES, m/z): 415 [M+H]⁺. ¹H NMR (300 MHz, DMSO-d₆) δ 11.37 (s, 1H), 9.92 (d, J=1.8 Hz, 1H), 9.60 (s, 2H), 8.95 (s, 1H), 8.57 (s, 1H), 8.24 (s, 1H), 8.02 (d, J=8.0 Hz, 1H), 6.60 (d, J=8.1 Hz, 1H), 4.33 (s, 3H), 3.66 (t, 4H), 3.31 (t, 4H), 2.45 (s, 3H).

### Example 92: Synthesis of Compound 359

### Synthesis of Intermediate C117

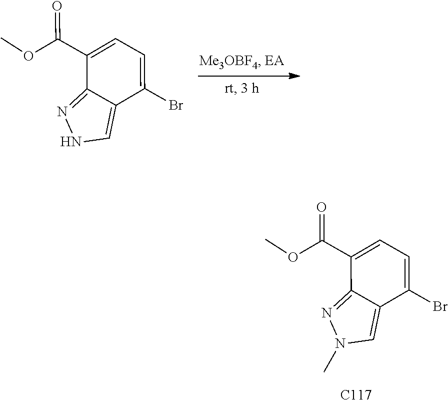

To a stirred solution of methyl 4-bromo-2H-indazole-7-carboxylate (5.0 g, 19.602 mmol, 1 equiv) in ethyl acetate (150 mL) was added tetrafluoroboranuide; trimethyloxidanium (14.50 g, 98.010 mmol, 5 equiv) at room temperature. The resulting mixture was stirred for 3 h at room temperature, then diluted with ethyl acetate (150 mL) and washed with water (3×200 mL). The organic phase was dried over anhydrous Na₂SO₄, and filtered. After filtration, the filtrate was concentrated under reduced pressure to afford methyl 4-bromo-2-methylindazole-7-carboxylate (4.8 g, 91%) as a solid. LCMS (ES, m/z): 269[M+H]⁺. ¹H NMR (300 MHz, DMSO-d₆) δ 8.62 (s, 1H), 7.84 (d, J=7.6 Hz, 1H), 7.42 (d, J=7.7 Hz, 1H), 4.25 (s, 3H), 3.89 (s, 3H).

### Synthesis of Intermediate C118

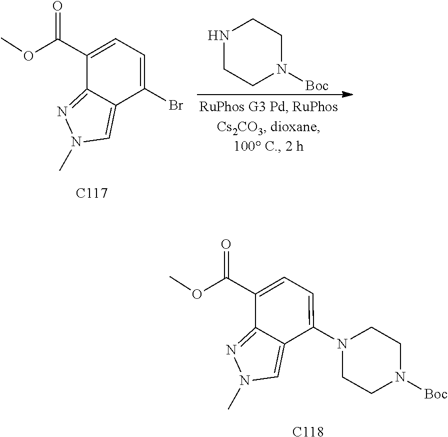

To a stirred mixture of methyl 4-bromo-2-methylindazole-7-carboxylate (4.5 g, 16.723 mmol, 1 equiv) and tert-butyl piperazine-1-carboxylate (6.23 g, 33.446 mmol, 2 equiv) in dioxane (90 mL) was added Cs2CO3 (16.35 g, 50.169 mmol, 3 equiv), RuPhos (1.56 g, 3.345 mmol, 0.2 equiv), and RuPhos Palladacycle Gen.3 (1.40 g, 1.672 mmol, 0.1 equiv) at room temperature under nitrogen atmosphere. The resulting mixture was stirred for 2 h at 100° C. under nitrogen atmosphere, then concentrated under vacuum to give a residue. The residue was purified by silica gel column chromatography, eluted with PE/EA (1:1) to afford methyl 4-[4-(tert-butoxycarbonyl)piperazin-1-yl]-2-methylindazole-7-carboxylate (5.2 g, 83%) as a solid. LCMS (ES, m/z): 375[M+H]⁺.

### Synthesis of Intermediate C119

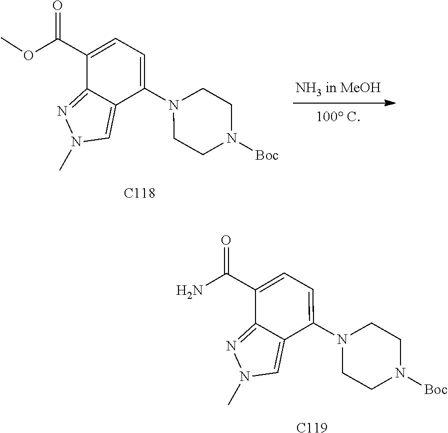

A mixture of methyl 4-[4-(tert-butoxycarbonyl)piperazin-1-yl]-2-methylindazole-7-carboxylate (2.5 g, 6.677 mmol, 1 equiv) and NH₃(g) in methanol (70 mL) was stirred for 2 days at 100° C. The resulting mixture was concentrated under reduced pressure to give a residue. The residue was purified by silica gel column chromatography, eluted with PE/EA (1:1) to afford tert-butyl 4-(7-carbamoyl-2-methylindazol-4-yl)piperazine-1-carboxylate (1.35 g, 56%) as a solid. LCMS (ES, m/z): 360 [M+H]⁺.

### Synthesis of Intermediate C120

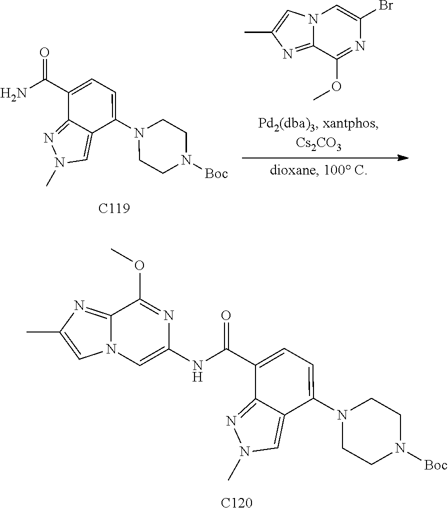

To a mixture of tert-butyl 4-(7-carbamoyl-2-methylindazol-4-yl)piperazine-1-carboxylate (60 mg, 0.167 mmol, 1 equiv), 6-bromo-8-methoxy-2-methylimidazo[1,2-a]pyrazine (48.49 mg, 0.200 mmol, 1.2 equiv), and Cs₂CO₃ (108.78 mg, 0.334 mmol, 2 equiv) in dioxane (2 mL) was added Xantphos (19.32 mg, 0.033 mmol, 0.2 equiv) and Pd₂(dba)₃ (15.29 mg, 0.017 mmol, 0.1 equiv). The reaction mixture was stirred overnight at 100° C. under a nitrogen atmosphere, then concentrated under reduced pressure to give a residue. The residue was purified by silica gel column chromatography, eluted with PE/EA (1:2) to afford tert-butyl 4-[7-({8-methoxy-2-methylimidazo[1,2-a]pyrazin-6-yl}carbamoyl)-2-methylindazol-4-yl]piperazine-1-carboxylate (35 mg, 40%) as a solid. LCMS (ES, m/z): 521 [M+H]⁺.

### Synthesis of Compound 359

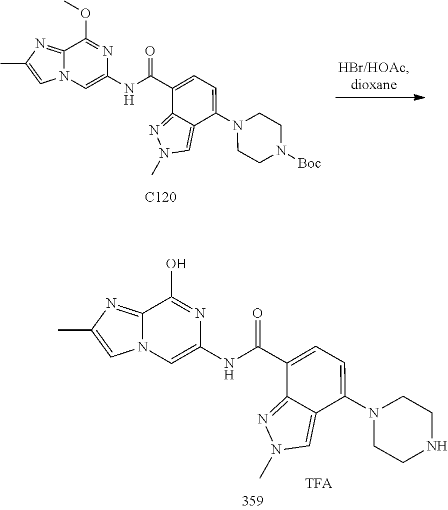

A solution of tert-butyl 4-[7-({8-methoxy-2-methylimidazo[1,2-a]pyrazin-6-yl}carbamoyl)-2-methylindazol-4-yl]piperazine-1-carboxylate (20 mg, 0.038 mmol, 1 equiv) in 1,4-dioxane was treated with HBr in AcOH (0.5 mL, 17.117 mmol, 445.56 equiv). The reaction mixture was stirred for 2 h at 80° C., then concentrated under reduced pressure to give a residue. The residue was purified by Prep-HPLC (Condition 9, Gradient 1) to afford N-{8-hydroxy-2-methylimidazo[1,2-a]pyrazin-6-yl}-2-methyl-4-(piperazin-1-yl) indazole-7-carboxamide; trifluoroacetic acid (6.5 mg, 31%) as a solid. LCMS (ES, m/z): 407 [M+H]⁺. ¹H NMR (400 MHz, DMSO-d₆) δ 10.94 (s, 1H), 8.90 (s, 3H), 8.33 (s, 1H), 8.01 (d, J=8.0 Hz, 1H), 7.78 (s, 1H), 6.61 (d, J=8.1 Hz, 1H), 4.30 (s, 3H), 3.62 (t, 4H), 3.35 (t, 4H), 2.34 (s, 3H). ¹⁹F NMR (400 MHz, DMSO-d₆) δ −73.89.

### Example 93: Synthesis of Compound 332

### Synthesis of Intermediate C121

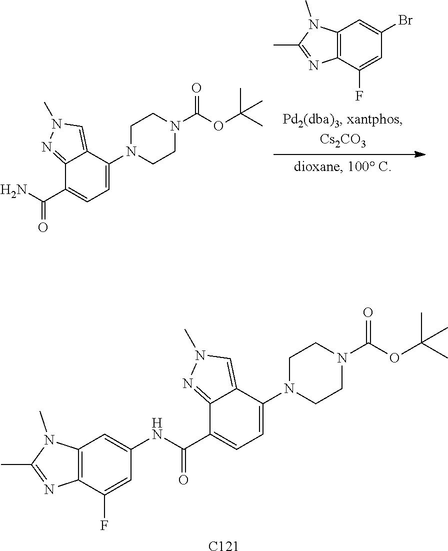 [SMILES: Cn1cc2c(N3CCN(C(=O)OC(C)(C)C)CC3)ccc(C(N)=O)c2n1 ; Cc1nc2c(F)cc(Br)cc2n1C ; Cc1nc2c(F)cc(NC(=O)c3ccc(N4CCN(C(=O)OC(C)(C)C)CC4)c4cn(C)nc34)cc2n1C ; "C121"]

To a mixture of tert-butyl 4-(7-carbamoyl-2-methylindazol-4-yl)piperazine-1-carboxylate (60 mg, 0.167 mmol, 1 equiv), 6-bromo-4-fluoro-1,2-dimethyl-1,3-benzodiazole (48.69 mg, 0.200 mmol, 1.2 equiv), and Cs₂CO₃ (108.78 mg, 0.334 mmol, 2 equiv) in dioxane (2 mL) was added Xantphos (19.32 mg, 0.033 mmol, 0.2 equiv) and Pd₂(dba)₃ (15.29 mg, 0.017 mmol, 0.1 equiv). The reaction mixture was stirred overnight at 100° C. under a nitrogen atmosphere, then concentrated under reduced pressure to give a residue. The residue was purified by silica gel column chromatography, eluted with PE/EA (1:2) to afford tert-butyl 4-{7-[(7-fluoro-2,3-dimethyl-1,3-benzodiazol-5-yl)carbamoyl]-2-methylindazol-4-yl}piperazine-1-carboxylate (43 mg, 49%) as a solid. LCMS (ES, m/z): 522[M+H]⁺.

### Synthesis of Compound 332

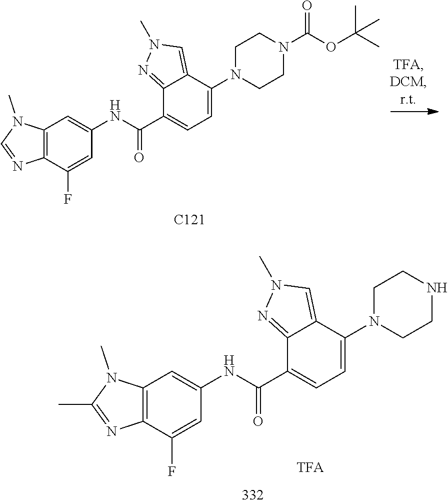

A solution of tert-butyl 4-{7-[(7-fluoro-2,3-dimethyl-1,3-benzodiazol-5-yl)carbamoyl]-2-methylindazol-4-yl}piperazine-1-carboxylate (41 mg, 0.079 mmol, 1 equiv) in DCM was added TFA (0.5 mL, 6.732 mmol, 85.64 equiv). The reaction mixture was stirred for 1 h at room temperature, then concentrated under vacuum to give a residue. The residue was purified by Prep-HPLC (Condition 10, Gradient 1, Gradient 2) to afford N-(7-fluoro-2,3-dimethyl-1,3-benzodiazol-5-yl)-2-methyl-4-(piperazin-1-yl) indazole-7-carboxamide; trifluoroacetic acid (26.9 mg, 64%) as a solid. LCMS (ES, m/z): 422 [M+H]⁺. ¹H NMR (300 MHz, DMSO-d₆) δ 11.34 (s, 1H), 8.96 (s, 2H), 8.91 (s, 1H), 8.05 (d, J=8.1 Hz, 1H), 8.00 (s, 1H), 7.55 (d, J=12.4 Hz, 1H), 6.63 (d, J=8.1 Hz, 1H), 4.34 (s, 3H), 3.81 (s, 3H), 3.59 (t, 4H), 3.36 (t, 4H), 2.63 (s, 3H). ¹⁹F NMR (300 MHz, DMSO-d₆) δ −74.09, −128.50.

### Example 94: Synthesis of Compound 343

### Synthesis of Intermediate C122

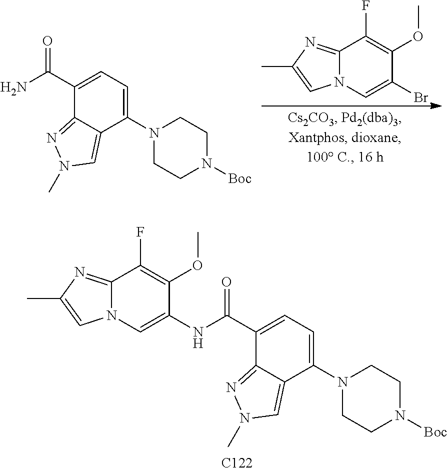

To a mixture of tert-butyl 4-(7-carbamoyl-2-methylindazol-4-yl)piperazine-1-carboxylate (100 mg, 0.278 mmol, 1 equiv) and 6-bromo-8-fluoro-7-methoxy-2-methylimidazo[1,2-a]pyridine (86.50 mg, 0.334 mmol, 1.2 equiv) in dioxane (2 mL) was added Cs₂CO₃ (271.95 mg, 0.834 mmol, 3 equiv), XantPhos (16.10 mg, 0.028 mmol, 0.1 equiv), and Pd₂(dba)₃CHCl₃ (14.40 mg, 0.014 mmol, 0.05 equiv). The reaction mixture was stirred for 16 h at 100° C. under a nitrogen atmosphere, then concentrated under reduced pressure to give a residue. The residue was purified by Prep-TLC/silica gel column chromatography, eluted with PE/THF (60%) to afford tert-butyl 4-[7-({8-fluoro-7-methoxy-2-methylimidazo[1,2-a]pyridin-6-yl}carbamoyl)-2-methylindazol-4-yl]piperazine-1-carboxylate (50 mg, 33%) as a solid. LCMS (ES, m/z): 538 [M+H]⁺.

### Synthesis of Compound 343

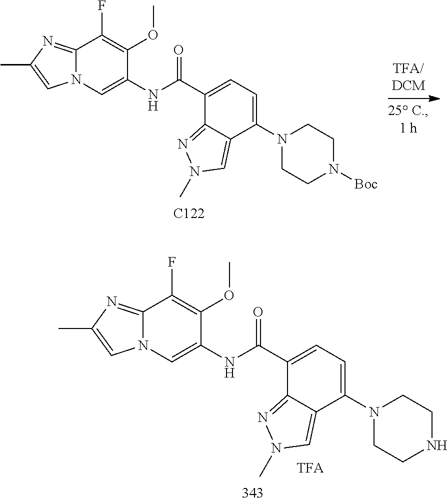

A mixture of tert-butyl 4-[7-({8-fluoro-7-methoxy-2-methylimidazo[1,2-a]pyridin-6-yl}carbamoyl)-2-methylindazol-4-yl]piperazine-1-carboxylate (50 mg, 0.093 mmol, 1 equiv), TFA (1 mL, 13.463 mmol), and DCM (3 mL) was stirred for 1 h at 25° C. The resulting mixture was concentrated under reduced pressure to give a residue. The residue was purified by Prep-HPLC (Condition 10, Gradient 1, Gradient 2) to afford N-{8-fluoro-7-methoxy-2-methylimidazo[1,2-a]pyridin-6-yl}-2-methyl-4-(piperazin-1-yl) indazole-7-carboxamide (35 mg, 86%) as a solid. LCMS (ES, m/z): 438 [M+H]⁺. ¹H NMR (300 MHz, DMSO-d₆) δ 11.73 (s, 1H), 9.79 (s, 1H), 8.94 (s, 1H), 8.15-8.04 (m, 2H), 6.64-6.62 (m, 1H), 4.71 (s, 3H), 4.32 (s, 3H), 3.76-6.60 (m, 4H), 3.42-3.19 (m, 4H) 2.44 (s, 3H).

### Example 95: Synthesis of Compound 319

### Synthesis of Intermediate C123

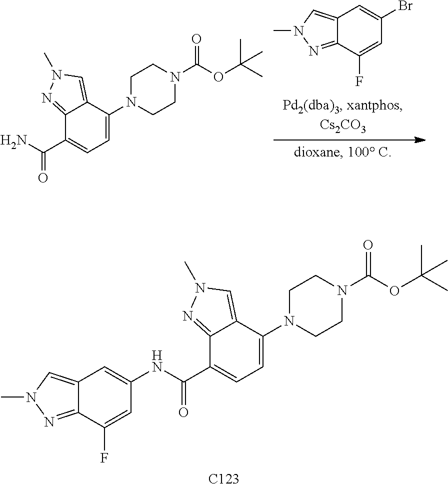

To a mixture of tert-butyl 4-(7-carbamoyl-2-methylindazol-4-yl)piperazine-1-carboxylate (60 mg, 0.167 mmol, 1 equiv) and 5-bromo-7-fluoro-2-methylindazole (45.88 mg, 0.200 mmol, 1.2 equiv) in dioxane (2 mL) was added Cs₂CO₃ (108.78 mg, 0.334 mmol, 2 equiv) and Xantphos (19.32 mg, 0.033 mmol, 0.2 equiv). The reaction mixture was stirred for 1 h at 100° C. under a nitrogen atmosphere, then concentrated under reduced pressure to give a residue. The residue was purified by silica gel column chromatography, eluted with PE/EA (1:2) to afford tert-butyl 4-{7-[(7-fluoro-2-methylindazol-5-yl)carbamoyl]-2-methylindazol-4-yl}piperazine-1-carboxylate (35 mg, 41%) as a solid. LCMS (ES, m/z): 508 [M+H]⁺.

### Synthesis of Compound 319

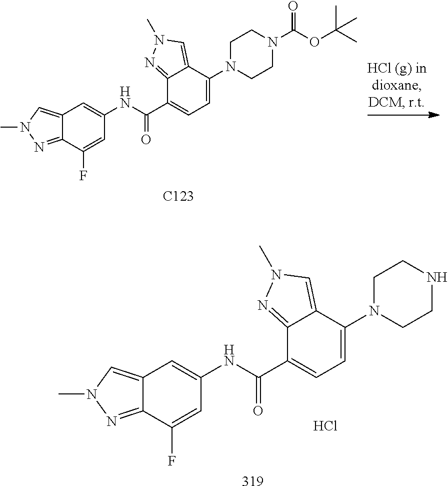

To a solution of tert-butyl 4-{7-[(7-fluoro-2-methylindazol-5-yl)carbamoyl]-2-methyl-octahydroindazol-4-yl}piperazine-1-carboxylate (30 mg, 0.058 mmol, 1 equiv) in DCM was added HCl (gas) in 1,4-dioxane (0.5 mL, 16.456 mmol, 282.85 equiv). The reaction mixture was stirred for 1 h at room temperature, then concentrated under reduced pressure to give a residue. The residue was purified by Prep-HPLC (Condition 11, Gradient 1) to afford N-(7-fluoro-2-methylindazol-5-yl)-2-methyl-4-(piperazin-1-yl) indazole-7-carboxamide hydrochloride (12.7 mg, 49%) as a solid. LCMS (ES, m/z): 408 [M+H]⁺. ¹H NMR (300 MHz, DMSO-d₆) δ 11.17 (s, 1H), 9.27 (s, 2H), 8.90 (s, 1H), 8.44 (d, J=2.9 Hz, 1H), 8.08 (d, J=1.6 Hz, 1H), 8.03 (d, J=7.9 Hz, 1H), 7.44 (dd, J=13.2, 1.6 Hz, 1H), 6.61 (d, J=8.1 Hz, 1H), 4.32 (s, 3H), 4.20 (s, 3H), 3.60 (t, 4H), 3.33 (t, 4H). ¹⁹F NMR (300 MHz, DMSO-d₆) δ −128.029.

### Example 96: Synthesis of Compound 320

### Synthesis of Intermediate C124

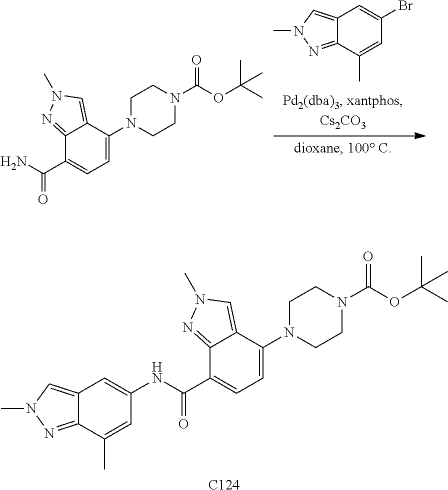 [SMILES: Cn1cc2c(N3CCN(C(=O)OC(C)(C)C)CC3)ccc(C(N)=O)c2n1 ; Cc1cc(Br)cc2cn(C)nc12 ; Cc1cc(NC(=O)c2ccc(N3CCN(C(=O)OC(C)(C)C)CC3)c3cn(C)nc23)cc2cn(C)nc12 ; "C124"]

To a solution of tert-butyl 4-(7-carbamoyl-2-methylindazol-4-yl)piperazine-1-carboxylate (60 mg, 0.167 mmol, 1 equiv) and 5-bromo-2,7-dimethylindazole (45.09 mg, 0.200 mmol, 1.2 equiv) in dioxane (2 mL) was added Cs₂CO₃ (108.78 mg, 0.334 mmol, 2 equiv), Xantphos (19.32 mg, 0.033 mmol, 0.2 equiv), and Pd₂(dba)₃ (15.29 mg, 0.017 mmol, 0.1 equiv). The reaction mixture was stirred overnight at 100° C. under a nitrogen atmosphere, then concentrated under reduced pressure to give a residue. The residue was purified by Prep-TLC/silica gel column chromatography, eluted with PE/EA (1:2) to afford tert-butyl 4-{7-[(2,7-dimethylindazol-5-yl)carbamoyl]-2-methylindazol-4-yl}piperazine-1-carboxylate (36 mg, 43%) as a solid. LCMS (ES, m/z): 504[M+H]⁺.

### Synthesis of Compound 320

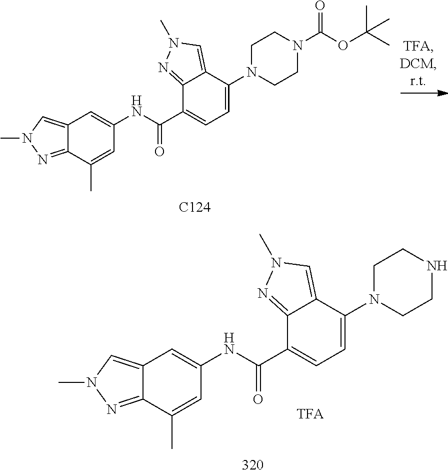

A solution of tert-butyl 4-{7-[(2,7-dimethylindazol-5-yl)carbamoyl]-2-methylindazol-4-yl}piperazine-1-carboxylate (35 mg, 0.069 mmol, 1 equiv) in DCM was treated with TFA (0.5 mL, 6.732 mmol, 96.86 equiv). The reaction mixture was stirred for 1 h at room temperature, then concentrated under vacuum to give a residue. The residue was purified by Prep-HPLC (Condition 10, Gradient 3) to afford N-(2,7-dimethylindazol-5-yl)-2-methyl-4-(piperazin-1-yl) indazole-7-carboxamide; trifluoroacetic acid (19.3 mg, 53%) as a solid. LCMS (ES, m/z): 404 [M+H]⁺. ¹H NMR (300 MHz, DMSO-d₆) δ 11.07 (s, 1H), 8.88 (m, 3H), 8.27 (s, 1H), 8.21 (s, 1H), 8.03 (d, J=7.9 Hz, 1H), 7.15 (s, 1H), 6.61 (d, J=8.0 Hz, 1H), 4.33 (s, 3H), 4.16 (s, 3H), 3.57 (t, 4H), 3.36 (t, 4H), 2.54 (s, 3H). ¹⁹F NMR (300 MHz, DMSO-d₆) δ −74.21.

### Example 97: Synthesis of Compound 321

### Synthesis of Intermediate C125

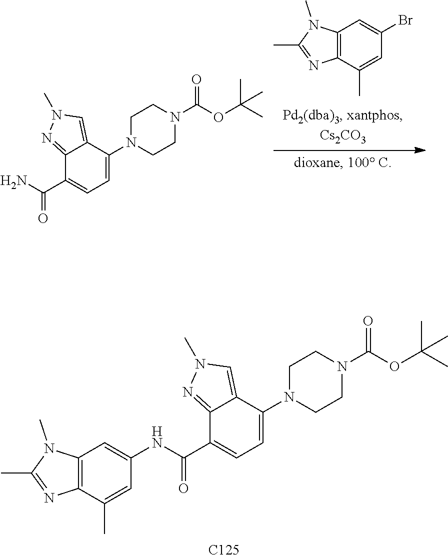 [SMILES: Cn1cc2c(N3CCN(C(=O)OC(C)(C)C)CC3)ccc(C(N)=O)c2n1 ; Cc1cc(Br)cc2c1nc(C)n2C ; Cc1cc(NC(=O)c2ccc(N3CCN(C(=O)OC(C)(C)C)CC3)c3cn(C)nc23)cc2c1nc(C)n2C ; "C125"]

To a mixture of tert-butyl 4-(7-carbamoyl-2-methylindazol-4-yl)piperazine-1-carboxylate (60 mg, 0.167 mmol, 1 equiv) and 6-bromo-1,2,4-trimethyl-1,3-benzodiazole (47.90 mg, 0.200 mmol, 1.2 equiv) in dioxane (2 mL) was added Cs₂CO₃ (108.78 mg, 0.334 mmol, 2 equiv), Xantphos (19.32 mg, 0.033 mmol, 0.2 equiv), and Pd₂(dba)₃ (15.29 mg, 0.017 mmol, 0.1 equiv). The reaction mixture was stirred overnight at 100° C. under a nitrogen atmosphere, then concentrated under reduced pressure to give a residue. The residue was purified by silica gel column chromatography, eluted with PE/EA (1:2) to afford tert-butyl 4-{2-methyl-7-[(2,3,7-trimethyl-1,3-benzodiazol-5-yl)carbamoyl]indazol-4-yl}piperazine-1-carboxylate (51 mg, 59%) as a solid. LCMS (ES, m/z): 518[M+H]⁺.

### Synthesis of Compound 321

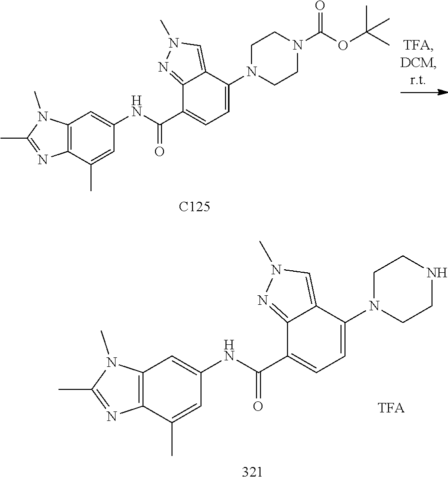

A solution of tert-butyl 4-{2-methyl-7-[(2,3,7-trimethyl-1,3-benzodiazol-5-yl)carbamoyl]indazol-4-yl}piperazine-1-carboxylate (50 mg, 0.097 mmol, 1 equiv) in DCM (1.0 mL) was treated with TFA (0.5 mL, 6.732 mmol, 69.69 equiv). The reaction mixture was stirred for 1 h at room temperature, then concentrated under reduced pressure to give a residue. The residue was purified by Prep-HPLC (Condition 10, Gradient 3) to afford 2-methyl-4-(piperazin-1-yl)-N-(2,3,7-trimethyl-1,3-benzodiazol-5-yl) indazole-7-carboxamide trifluoroacetate (26.6 mg, 51%) as a solid. LCMS (ES, m/z): 418 [M+H]⁺. ¹H NMR (300 MHz, DMSO-d₆) δ 11.38 (s, 1H), 9.00 (s, 2H), 8.93 (s, 1H), 8.41 (s, 1H), 8.06 (d, J=8.0 Hz, 1H), 7.51 (s, 1H), 6.63 (d, J=8.1 Hz, 1H), 4.34 (s, 3H), 3.91 (s, 3H), 3.59 (t, 4H), 3.34 (t, 4H), 2.81 (s, 3H), 2.61 (s, 3H).

### Example 98: Synthesis of Compound 322

### Synthesis of Intermediate C126

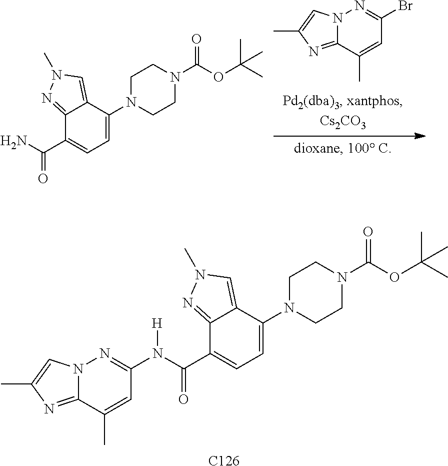

To a mixture of tert-butyl 4-(7-carbamoyl-2-methylindazol-4-yl)piperazine-1-carboxylate (60 mg, 0.167 mmol, 1 equiv) and 6-bromo-2,8-dimethylimidazo[1,2-b]pyridazine (45.29 mg, 0.200 mmol, 1.2 equiv) in dioxane (2 mL, 23.608 mmol, 141.42 equiv) was added Cs₂CO₃ (108.78 mg, 0.334 mmol, 2 equiv), Xantphos (19.32 mg, 0.033 mmol, 0.2 equiv), and Pd₂(dba)₃ (15.29 mg, 0.017 mmol, 0.1 equiv). The reaction mixture was stirred overnight at 100° C. under a nitrogen atmosphere, then concentrated under reduced pressure to give a residue. The residue was purified by silica gel column chromatography, eluted with PE/EA (1:2) to afford tert-butyl 4-[7-({2,8-dimethylimidazo[1,2-b]pyridazin-6-yl}carbamoyl)-2-methylindazol-4-yl]piperazine-1-carboxylate (28 mg, 33%) as a solid. LCMS (ES, m/z): 505 [M+H]⁺.

### Synthesis of Compound 322

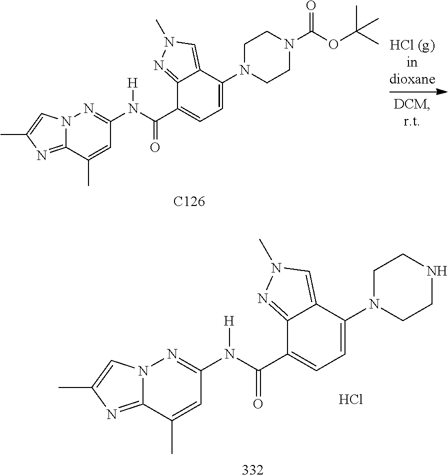

A solution of tert-butyl 4-[7-({2,8-dimethylimidazo[1,2-b]pyridazin-6-yl}carbamoyl)-2-methylindazol-4-yl]piperazine-1-carboxylate (28 mg, 0.055 mmol, 1 equiv) in DCM was treated with HCl (gas) in 1,4-dioxane (0.5 mL, 54.855 mmol, 988.55 equiv). The reaction mixture was stirred for 1 h at room temperature, then concentrated under reduced pressure to give a residue. The residue was purified by Prep-HPLC (Condition 11, Gradient 1) to afford N-{2,8-dimethylimidazo[1,2-b]pyridazin-6-yl}-2-methyl-4-(piperazin-1-yl) indazole-7-carboxamide hydrochloride (14.2 mg, 58%) as a solid. LCMS (ES, m/z): 405 [M+H]⁺. ¹H NMR (300 MHz, DMSO-d₆) δ 11.79 (s, 1H), 9.56 (s, 2H), 9.00 (s, 1H), 8.65 (s, 1H), 8.37 (s, 1H), 8.10 (d, J=8.1, 1.5 Hz, 1H), 6.64 (d, J=8.2 Hz, 1H), 4.29 (s, 3H), 3.70 (t, 4H), 3.32 (t, 4H), 2.72 (s, 3H), 2.55 (s, 3H).

### Example 99: Synthesis of Compound 323

### Synthesis of Intermediate C127

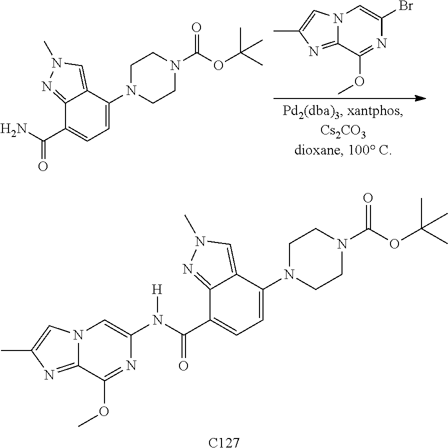

To a mixture of tert-butyl 4-(7-carbamoyl-2-methylindazol-4-yl)piperazine-1-carboxylate (60 mg, 0.167 mmol, 1 equiv) and 6-bromo-8-methoxy-2-methylimidazo[1,2-a]pyrazine (48.49 mg, 0.200 mmol, 1.2 equiv) in dioxane (2 mL) was added Cs₂CO₃ (108.78 mg, 0.334 mmol, 2 equiv), Xantphos (19.32 mg, 0.033 mmol, 0.2 equiv), and Pd₂(dba)₃ (15.29 mg, 0.017 mmol, 0.1 equiv). The reaction mixture was stirred overnight at 100° C. under a nitrogen atmosphere, then concentrated under reduced pressure to give a residue. The residue was purified by silica gel column chromatography, eluted with PE/EA (1:2) to afford tert-butyl 4-[7-({8-methoxy-2-methylimidazo[1,2-a]pyrazin-6-yl}carbamoyl)-2-methylindazol-4-yl]piperazine-1-carboxylate (35 mg, 40%) as a solid. LCMS (ES, m/z): 521 [M+H]⁺.

### Synthesis of Compound 323

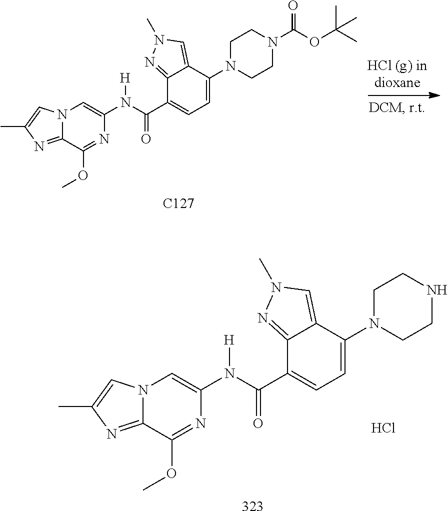

A solution of tert-butyl 4-[7-({8-methoxy-2-methylimidazo[1,2-a]pyrazin-6-yl}carbamoyl)-2-methylindazol-4-yl]piperazine-1-carboxylate (20 mg, 0.038 mmol, 1 equiv) in 1,4-dioxane was treated with HBr in AcOH (0.5 mL, 17.117 mmol, 445.56 equiv). The reaction mixture was stirred for 2 h at 80° C. The resulting mixture was concentrated under reduced pressure to give a residue. The residue was purified by Prep-HPLC (Condition 11, Gradient 1) to afford N-{8-hydroxy-2-methylimidazo[1,2-a]pyrazin-6-yl}-2-methyl-4-(piperazin-1-yl) indazole-7-carboxamide; trifluoroacetic acid (6.5 mg, 31%) as a solid. LCMS (ES, m/z): 421 [M+H]⁺. ¹H NMR (300 MHz, DMSO-d₆) δ 11.48 (s, 1H), 9.40 (brs, 2H), 9.23 (s, 1H), 8.95 (s, 1H), 8.21 (s, 1H), 8.07 (d, J=8.0 Hz, 1H), 6.63 (d, J=8.1 Hz, 1H), 4.29 (s, 3H), 4.21 (s, 3H), 3.66 (t, 4H), 3.32 (t, 4H), 2.46 (s, 3H).

### Example 100: Synthesis of Compound 324

### Synthesis of Intermediate C128

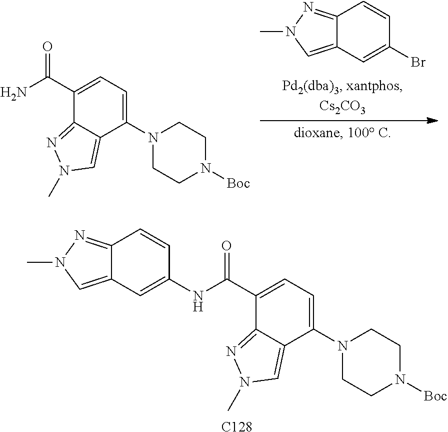 [SMILES: Cn1cc2c(N3CCN(C(=O)OC(C)(C)C)CC3)ccc(C(N)=O)c2n1 ; Cn1cc2cc(Br)ccc2n1 ; Cn1cc2cc(NC(=O)c3ccc(N4CCN(C(=O)OC(C)(C)C)CC4)c4cn(C)nc34)ccc2n1 ; "C128"]

To a solution of tert-butyl 4-(7-carbamoyl-2-methylindazol-4-yl) piperazine-1-carboxylate (60 mg, 0.167 mmol, 1 equiv), 5-bromo-2-methylindazole (52.85 mg, 0.251 mmol, 1.5 equiv) and Cs₂CO₃ (108.78 mg, 0.334 mmol, 2 equiv) in dioxane (2 mL) was added XantPhos (19.32 mg, 0.033 mmol, 0.2 equiv) and Pd₂(dba)₃ (15.29 mg, 0.017 mmol, 0.1 equiv). The reaction mixture was stirred for 30 seconds at room temperature under nitrogen atmosphere, then overnight at 100° C. under nitrogen atmosphere. The resulting mixture was concentrated under reduced pressure to give a residue. The residue was purified by silica gel column chromatography, eluted with PE/EA (2:1) to afford tert-butyl 4-{2-methyl-7-[(2-methylindazol-5-yl) carbamoyl]indazol-4-yl} piperazine-1-carboxylate (30 mg, 33%) as a solid. LCMS (ES, m/z): 490 [M+H]⁺.

### Synthesis of Compound 324

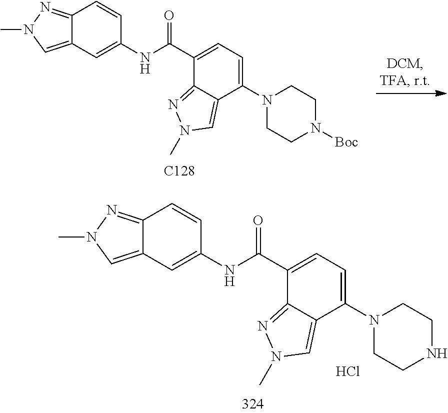 [SMILES: Cn1cc2cc(NC(=O)c3ccc(N4CCN(C(=O)OC(C)(C)C)CC4)c4cn(C)nc34)ccc2n1 ; "C128" ; Cn1cc2cc(NC(=O)c3ccc(N4CCNCC4)c4cn(C)nc34)ccc2n1 ; Cl]

To a mixture of tert-butyl 4-{2-methyl-7-[(2-methylindazol-5-yl) carbamoyl] indazol-4-yl}piperazine-1-carboxylate (30 mg, 0.061 mmol, 1 equiv) in DCM (1.0 mL) was added TFA (0.5 mL, 6.732 mmol, 109.85 equiv). The reaction mixture was stirred for 1 hour at room temperature, then concentrated under reduced pressure to give a residue. The residue was purified by Prep-HPLC (Condition 11, Gradient 1) to afford 2-methyl-N-(2-methylindazol-5-yl)-4-(piperazin-1-yl) indazole-7-carboxamide hydrochloride (7.6 mg, 28%) as a solid. LCMS (ES, m/z): 390 [M+H]. ¹H NMR (300 MHz, DMSO-d₆) δ 11.15 (s, 1H), 9.40 (s, 2H), 8.90 (s, 1H), 8.38 (d, J=1.9 Hz, 1H), 8.31 (s, 1H), 8.03 (d, J=7.9 Hz, 1H), 7.63 (d, J=9.1 Hz, 1H), 7.40 (dd, J=9.1, 2.0 Hz, 1H), 6.61 (d, J=8.0 Hz, 1H), 4.32 (s, 3H), 4.16 (s, 3H), 3.60 (t, 4H), 3.32 (t, 4H).

### Example 101: Synthesis of Compound 325

### Synthesis of Intermediate C129

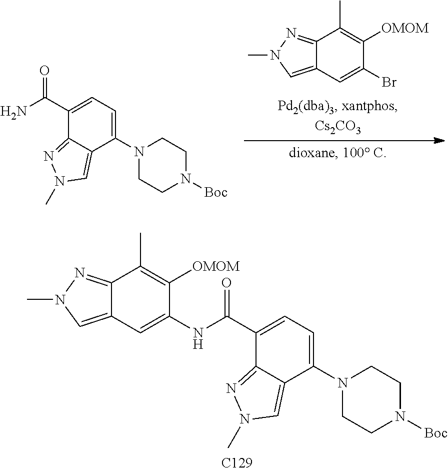

To a solution of tert-butyl 4-(7-carbamoyl-2-methylindazol-4-yl)piperazine-1-carboxylate (60 mg, 0.167 mmol, 1.00 equiv), 5-bromo-6-(methoxymethoxy)-2,7-dimethylindazole (57.12 mg, 0.200 mmol, 1.2 equiv), and Cs₂CO₃ (108.78 mg, 0.334 mmol, 2.0 equiv) in dioxane (2 mL) was added XantPhos (19.32 mg, 0.033 mmol, 0.2 equiv) and Pd₂(dba)₃ (15.29 mg, 0.017 mmol, 0.10 equiv). The reaction mixture was stirred for 16 h at 100° C. under a nitrogen atmosphere, then concentrated under reduced pressure to give a residue. The residue was purified by silica gel column chromatography, eluted with PE/EA (1:2) to afford tert-butyl 4-(7-{[6-(methoxymethoxy)-2,7-dimethylindazol-5-yl]carbamoyl}-2-methylindazol-4-yl)piperazine-1-carboxylate (40 mg, 43%) as a solid. LCMS (ES, m/z): 564 [M+H]⁺.

### Synthesis of Compound 325

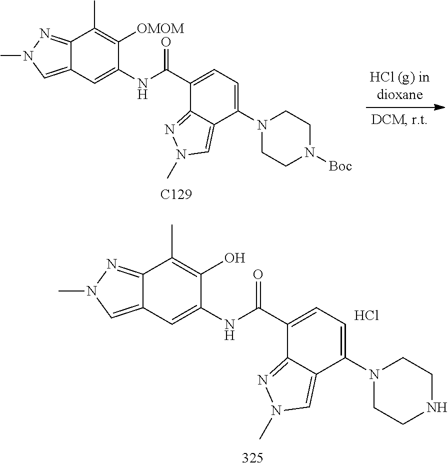

A solution of tert-butyl 4-(7-{[6-(methoxymethoxy)-2,7-dimethylindazol-5-yl]carbamoyl}-2-methylindazol-4-yl)piperazine-1-carboxylate (40 mg, 0.071 mmol, 1 equiv) in DCM was treated with HCl (gas) in 1,4-dioxane (0.3 mL, 32.913 mmol, 463.79 equiv). The reaction mixture was stirred for 1 h at room temperature, then concentrated under reduced pressure to give a residue. The residue was purified by Prep-HPLC (Condition 11, Gradient 1) to afford N-(6-hydroxy-2,7-dimethylindazol-5-yl)-2-methyl-4-(piperazin-1-yl) indazole-7-carboxamide hydrochloride (12.1 mg, 36%). LCMS (ES, m/z): 420 [M+H]⁺. ¹H NMR (300 MHz, DMSO-d₆) δ 11.60 (s, 1H), 9.22 (br, 3H), 8.86 (s, 1H), 8.61 (s, 1H), 8.17 (s, 1H), 8.05 (d, J=7.9 Hz, 1H), 6.60 (d, J=8.0 Hz, 1H), 4.29 (s, 3H), 4.10 (s, 3H), 3.44 (t, 4H), 3.34 (t, 4H), 2.43 (s, 3H).

### Example 102: Synthesis of Compound 315

### Synthesis of Intermediate C130

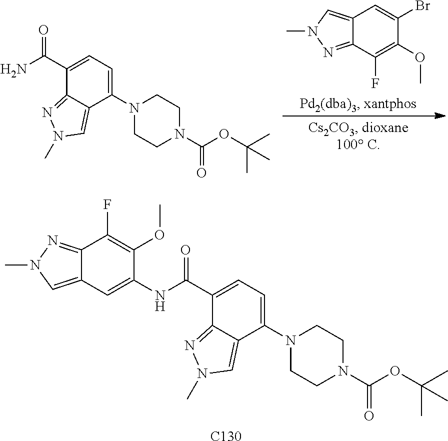 [SMILES: Cn1cc2c(N3CCN(C(=O)OC(C)(C)C)CC3)ccc(C(N)=O)c2n1 ; COc1c(Br)cc2cn(C)nc2c1F ; "Pd2(dba)3, xantphos" ; COc1c(NC(=O)c2ccc(N3CCN(C(=O)OC(C)(C)C)CC3)c3cn(C)nc23)cc2cn(C)nc2c1F ; "C130"]

To a mixture of tert-butyl 4-(7-carbamoyl-2-methylindazol-4-yl)piperazine-1-carboxylate (60 mg, 0.167 mmol, 1 equiv) and 5-bromo-7-fluoro-6-methoxy-2-methylindazole (51.90 mg, 0.200 mmol, 1.2 equiv) in dioxane (2 mL) was added Cs2CO3 (108.78 mg, 0.334 mmol, 2 equiv), Xantphos (19.32 mg, 0.033 mmol, 0.2 equiv), and Pd₂(dba)₃ (15.29 mg, 0.017 mmol, 0.1 equiv). The reaction mixture was stirred for 1 h at 100° C. under a nitrogen atmosphere, then concentrated under reduced pressure to give a residue. The residue was purified by silica gel column chromatography, eluted with PE/EA (1:2) to afford tert-butyl 4-{7-[(7-fluoro-6-methoxy-2-methylindazol-5-yl)carbamoyl]-2-methylindazol-4-yl}piperazine-1-carboxylate (32 mg, 36%) as a solid. LCMS (ES, m/z): 538 [M+H]⁺. Synthesis of Compound 315

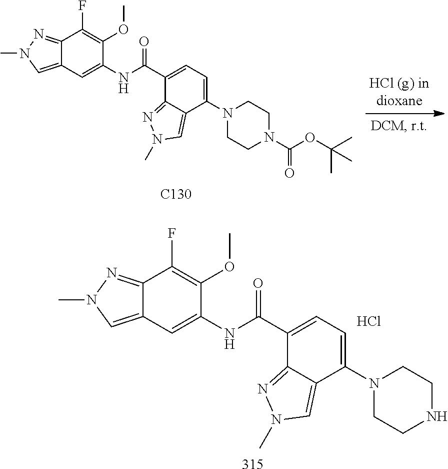

A solution of tert-butyl 4-{7-[(7-fluoro-6-methoxy-2-methylindazol-5-yl)carbamoyl]-2-methylindazol-4-yl}piperazine-1-carboxylate (32 mg, 0.060 mmol, 1 equiv) in DCM was treated with HCl (gas) in 1,4-dioxane (0.5 mL, 16.456 mmol, 276.47 equiv). The reaction mixture was stirred for 1 h at room temperature, then concentrated under reduced pressure to give a residue. The residue was purified by Prep-HPLC (Condition 11, Gradient 1) to afford N-(7-fluoro-6-methoxy-2-methylindazol-5-yl)-2-methyl-4-(piperazin-1-yl) indazole-7-carboxamide hydrochloride (13.5 mg, 47%) as a solid. LCMS (ES, m/z): 438 [M+H]⁺. ¹H NMR (300 MHz, DMSO-d₆) δ 11.58 (s, 1H), 9.29 (s, 2H), 8.90 (s, 1H), 8.69 (s, 1H), 8.40 (s, 1H), 8.06 (d, J=8.0 Hz, 1H), 6.61 (d, J=8.1 Hz, 1H), 4.31 (s, 3H), 4.18-4.11 (m, 6H), 3.60 (t, 4H), 3.32 (t, 4H). ¹⁹F NMR (300 MHz, DMSO-d₆) δ 149.08.

### Example 103: Synthesis of Compound 344

### Synthesis of Intermediate C131

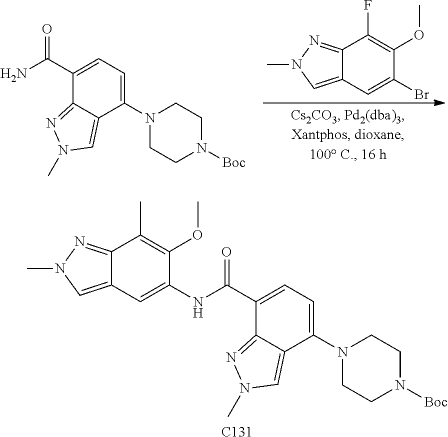 [SMILES: Cn1cc2c(N3CCN(C(=O)OC(C)(C)C)CC3)ccc(C(N)=O)c2n1 ; COc1c(Br)cc2cn(C)nc2c1F ; COc1c(NC(=O)c2ccc(N3CCN(C(=O)OC(C)(C)C)CC3)c3cn(C)nc23)cc2cn(C)nc2c1C ; "C131"]

To a mixture of tert-butyl 4-(7-carbamoyl-2-methylindazol-4-yl)piperazine-1-carboxylate (100 mg, 0.278 mmol, 1 equiv) and 6-bromo-8-fluoro-7-methoxy-2-methylimidazo[1,2-a]pyridine (86.50 mg, 0.334 mmol, 1.2 equiv) in dioxane (2 mL) was added Cs₂CO₃ (271.95 mg, 0.834 mmol, 3 equiv), XantPhos (16.10 mg, 0.028 mmol, 0.1 equiv), and Pd₂(dba)₃CHCl₃ (14.40 mg, 0.014 mmol, 0.05 equiv). The reaction mixture was stirred for 16 h at 100° C. under a nitrogen atmosphere, then concentrated under reduced pressure to give a residue. The residue was purified by Prep-TLC/silica gel column chromatography, eluted with PE/THF (60%) to afford tert-butyl 4-{7-[(6-methoxy-2,7-dimethylindazol-5-yl)carbamoyl]-2-methylindazol-4-yl}piperazine-1-carboxylate (53 mg, 36%) as a solid. LCMS (ES, m/z): 534 [M+H]⁺.

### Synthesis of Compound 344

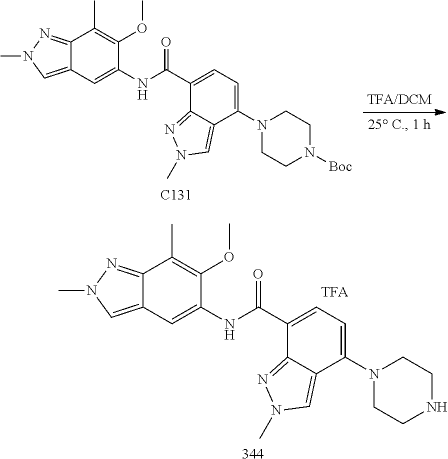

A mixture of tert-butyl 4-{7-[(6-methoxy-2,7-dimethylindazol-5-yl)carbamoyl]-2-methylindazol-4-yl}piperazine-1-carboxylate (50 mg, 0.094 mmol, 1 equiv) and TFA (1 mL, 13.463 mmol, 143.69 equiv) in DCM (3 mL) was stirred for 1 h at 25° C. The resulting mixture was concentrated under vacuum to give a residue. The residue was purified by Prep-HPLC (Condition 10, Gradient 1, Gradient 2, Gradient 4) to afford N-(6-methoxy-2,7-dimethylindazol-5-yl)-2-methyl-4-(piperazin-1-yl) indazole-7-carboxamide trifluoroacetate (37 mg, 91%) as a solid. LCMS (ES, m/z): 434 [M+H]⁺. ¹H NMR (300 MHz, DMSO-d₆) δ 11.51 (s, 1H), 8.87 (s, 1H), 8.75 (s, 1H), 8.25 (s, 1H), 8.07 (d, J=8.0 Hz, 1H), 6.62 (d, J=8.0 Hz, 1H), 4.32 (s, 3H), 4.14 (s, 3H), 3.94 (s, 3H), 3.62-3.53 (m, 4H), 3.43-3.19 (m, 4H), 2.50 (s, 3H).

### Example 104: Synthesis of Compound 333

### Synthesis of Intermediate C132

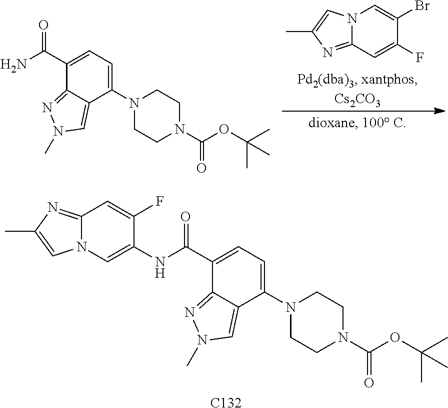

To a mixture of Cs₂CO₃ (108.78 mg, 0.334 mmol, 2 equiv) and 6-bromo-7-fluoro-2-methylimidazo[1,2-a]pyridine (45.88 mg, 0.200 mmol, 1.2 equiv) in dioxane (2 mL) was added Xantphos (19.32 mg, 0.033 mmol, 0.2 equiv) and Pd₂(dba)₃ (15.29 mg, 0.017 mmol, 0.1 equiv). The reaction mixture was stirred overnight at 100° C. under a nitrogen atmosphere, then concentrated under reduced pressure to give a residue. The residue was purified by silica gel column chromatography, eluted with PE/EA (1:2) to afford tert-butyl 4-[7-({7-fluoro-2-methylimidazo[1,2-a]pyridin-6-yl}carbamoyl)-2-methylindazol-4-yl]piperazine-1-carboxylate (45 mg, 53%) as a solid. LCMS (ES, m/z): 508 [M+H]⁺.

### Synthesis of Compound 333

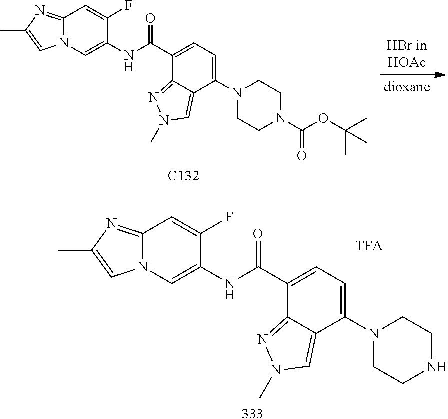

A solution of tert-butyl 4-[7-({7-fluoro-2-methylimidazo[1,2-a]pyridin-6-yl}carbamoyl)-2-methylindazol-4-yl]piperazine-1-carboxylate (41 mg, 0.081 mmol, 1 equiv) in DCM was treated with TFA (0.5 mL, 6.732 mmol, 83.33 equiv). The reaction mixture was stirred for 1 h at room temperature, then concentrated under reduced pressure to give a residue. The residue was purified by Prep-HPLC (Condition 10, Gradient 1, Gradient 2) to afford N-{7-fluoro-2-methylimidazo[1,2-a]pyridin-6-yl}-2-methyl-4-(piperazin-1-yl) indazole-7-carboxamide trifluoroacetate (25.6 mg, 61%) as a solid. LCMS (ES, m/z): 408 [M+H]⁺. ¹H NMR (300 MHz, DMSO-d₆) δ 11.59 (d, J=3.1 Hz, 1H), 9.98 (d, J=6.6 Hz, 1H), 9.04 (s, 2H), 8.96 (s, 1H), 8.16 (s, 1H), 8.08 (dd, J=9.1, 5.1 Hz, 2H), 6.64 (d, J=8.1 Hz, 1H), 4.27 (s, 3H), 3.64 (t, 4H), 3.27 (t, 4H), 2.45 (s, 3H). ¹⁹F NMR (300 MHz, DMSO-d₆) δ −73.71, −117.21.

### Example 105: Synthesis of Compound 334

### Synthesis of Intermediate C133

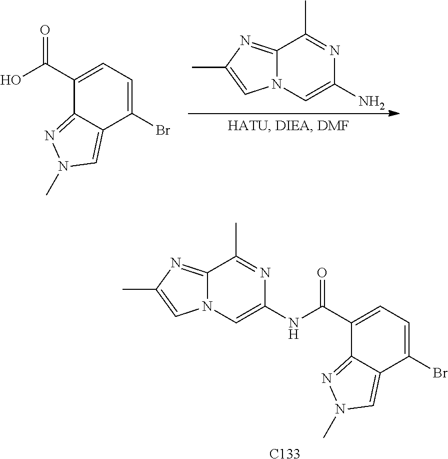 [SMILES: Cn1cc2c(Br)ccc(C(=O)O)c2n1 ; Cc1cn2cc(N)nc(C)c2n1 ; Cc1cn2cc(NC(=O)c3ccc(Br)c4cn(C)nc34)nc(C)c2n1 ; "C133"]

To a stirred mixture of 4-bromo-2-methylindazole-7-carboxylic acid (80 mg, 0.314 mmol, 1 equiv) and HATU (155.03 mg, 0.408 mmol, 1.3 equiv) in DMF (2 mL) was added DIEA (121.61 mg, 0.942 mmol, 3 equiv) and 2,8-dimethylimidazo[1,2-a]pyrazin-6-amine (66.13 mg, 0.408 mmol, 1.3 equiv) at room temperature. The resulting mixture was stirred overnight at room temperature. A precipitate formed that was collected by filtration. The resulting solid was dried under infrared light to afford 4-bromo-N-{2,8-dimethylimidazo[1,2-a]pyrazin-6-yl}-2-methylindazole-7-carboxamide (60 mg, 48%) as a solid. LCMS (ES, m/z): 399[M+H]⁺.

### Synthesis of Intermediate C134

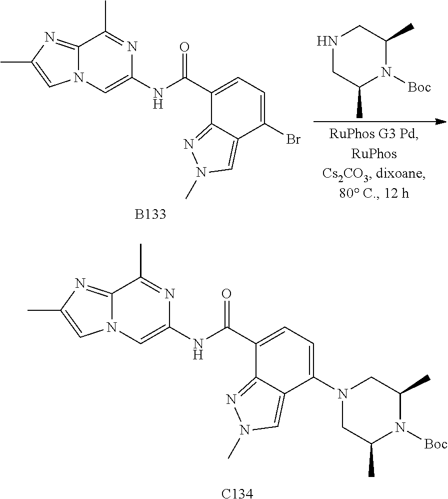 [SMILES: "B133" ; Cc1cn2cc(NC(=O)c3ccc(Br)c4cn(C)nc34)nc(C)c2n1 ; Cc1cn2cc(NC(=O)c3ccc(N4C[C@@H](C)N(C(=O)OC(C)(C)C)[C@@H](C)C4)c4cn(C)nc34)nc(C)c2n1 ; C[C@@H]1CNC[C@H](C)N1C(=O)OC(C)(C)C ; "C134"]

To a stirred mixture of 4-bromo-N-{2,8-dimethylimidazo[1,2-a]pyrazin-6-yl}-2-methylindazole-7-carboxamide (60 mg, 0.150 mmol, 1 equiv) and tert-butyl (2R,6S)-2,6-dimethylpiperazine-1-carboxylate (38.65 mg, 0.180 mmol, 1.2 equiv) in 1,4-dioxane (1.2 mL) was added Cs₂CO₃ (146.89 mg, 0.450 mmol, 3 equiv), RuPhos (14.03 mg, 0.030 mmol, 0.2 equiv), and RuPhos Palladacycle Gen.3 (12.57 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl (2R,6S)-4-[7-({2,8-dimethylimidazo[1,2-a]pyrazin-6-yl}carbamoyl)-2-methylindazol-4-yl]-2,6-dimethylpiperazine-1-carboxylate (70 mg, 66%) as a solid. LCMS (ES, m/z): 533[M+H]⁺.

### Synthesis of Compound 334

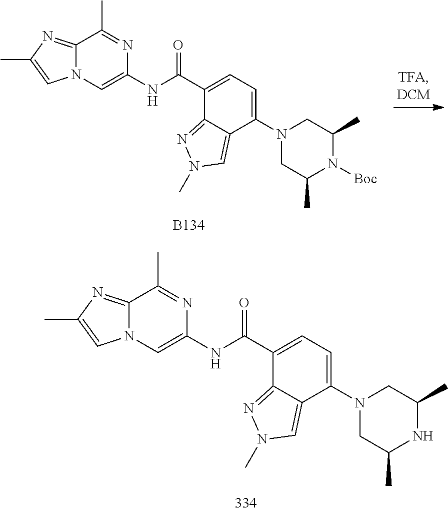

To a stirred solution of tert-butyl (2R,6S)-4-[7-({2,8-dimethylimidazo[1,2-a]pyrazin-6-yl}carbamoyl)-2-methylindazol-4-yl]-2,6-dimethylpiperazine-1-carboxylate (70 mg, 0.131 mmol, 1 equiv) in DCM (1 mL) was added TFA (1 mL, 13.463 mmol, 102.44 equiv) at room temperature. The resulting mixture was stirred for 1 h at room temperature, then concentrated under reduced pressure to give a residue. The residue was purified by Prep-HPLC (Condition 10, Gradient 4) to afford N-{2,8-dimethylimidazo[1,2-a]pyrazin-6-yl}-4-[(3R,5S)-3,5-dimethylpiperazin-1-yl]-2-methylindazole-7-carboxamide (11.3 mg, 20%) as a solid. LCMS (ES, m/z): 433 [M+H]⁺. ¹H NMR (400 MHz, DMSO-d₆) δ 11.34 (s, 1H), 9.36 (s, 1H), 9.17 (d, J=10.6 Hz, 1H), 8.94 (s, 1H), 8.59 (d, J=10.8 Hz, 1H), 8.11-8.05 (m, 2H), 6.66 (d, J=8.1 Hz, 1H), 4.31 (s, 3H), 4.03 (d, J=13.2 Hz, 2H), 3.00-2.89 (m, 2H), 2.76 (s, 3H), 2.43 (s, 3H), 1.33 (d, J=6.4 Hz, 6H).

### Example 106: Synthesis of Compound 347

### Synthesis of Intermediate C135

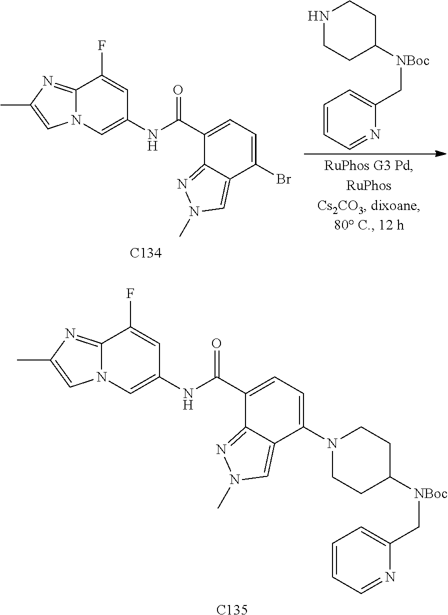 [SMILES: Cc1cn2cc(NC(=O)c3ccc(Br)c4cn(C)nc34)cc(F)c2n1 ; CC(C)(C)OC(=O)N(Cc1ccccn1)C1CCNCC1 ; "C134" ; Cc1cn2cc(NC(=O)c3ccc(N4CCC(N(Cc5ccccn5)C(=O)OC(C)(C)C)CC4)c4cn(C)nc34)cc(F)c2n1 ; "C135"]

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-(piperidin-4-yl)-N-(pyridin-2-ylmethyl)carbamate (52.16 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl N-{1-[7-({8-fluoro-2-methylimidazo[1,2-a]pyridin-6-yl}carbamoyl)-2-methylindazol-4-yl]piperidin-4-yl}-N-(pyridin-2-ylmethyl)carbamate (70 mg, 77%) as solid. LCMS (ES, m/z): 613[M+H]⁺.

### Synthesis of Compound 347

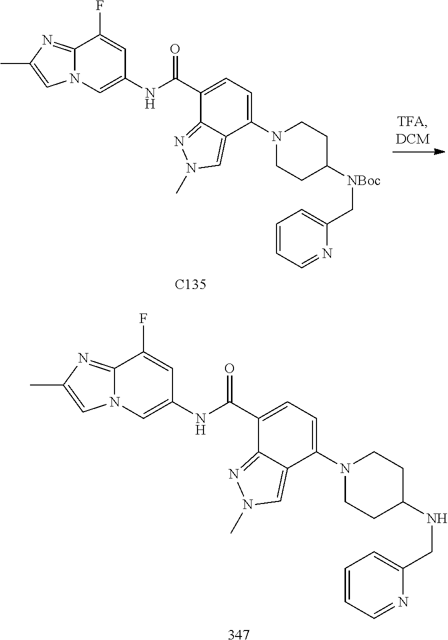 [SMILES: Cc1cn2cc(NC(=O)c3ccc(N4CCC(N(Cc5ccccn5)C(=O)OC(C)(C)C)CC4)c4cn(C)nc34)cc(F)c2n1 ; "C135" ; Cc1cn2cc(NC(=O)c3ccc(N4CCC(NCc5ccccn5)CC4)c4cn(C)nc34)cc(F)c2n1 ; "347"]

To a stirred solution of tert-butyl N-{1-[7-({8-fluoro-2-methylimidazo[1,2-a]pyridin-6-yl}carbamoyl)-2-methylindazol-4-yl]piperidin-4-yl}-N-(pyridin-2-ylmethyl)carbamate (70 mg, 0.114 mmol, 1 equiv) in DCM (1 mL) was added TFA (1 mL, 13.463 mmol, 117.84 equiv) at room temperature. The resulting mixture was stirred for 1 h at room temperature, then concentrated under reduced pressure to give a residue. The residue was purified by Prep-HPLC (Condition 2, Gradient 7) to afford N-{8-fluoro-2-methylimidazo[1,2-a]pyridin-6-yl}-2-methyl-4-{4-[(pyridin-2-ylmethyl)amino]piperidin-1-yl}indazole-7-carboxamide (13.1 mg, 22%) as a solid. LCMS (ES, m/z): 513 [M+H]⁺. ¹H NMR (400 MHz, DMSO-d₆) δ 11.07 (s, 1H), 9.21 (d, J=1.7 Hz, 1H), 8.78 (s, 1H), 8.51 (d, J=4.8 Hz, 1H), 7.97 (d, J=8.1 Hz, 1H), 7.90 (d, J=3.0 Hz, 1H), 7.77 (td, J=7.6, 1.8 Hz, 1H), 7.49 (d, J=7.8 Hz, 1H), 7.34 (dd, J=12.4, 1.7 Hz, 1H), 7.29-7.22 (m, 1H), 6.49 (d, J=8.2 Hz, 1H), 4.30 (s, 3H), 3.89 (d, J=9.2 Hz, 4H), 3.06 (t, J=11.8 Hz, 2H), 2.79-2.63 (m, 1H), 2.38-2.33 (m, 3H), 2.01 (d, J=12.3 Hz, 2H), 1.52 (d, J=11.4 Hz, 2H). Example 107: Synthesis of Compound 360

### Synthesis of Intermediate C136

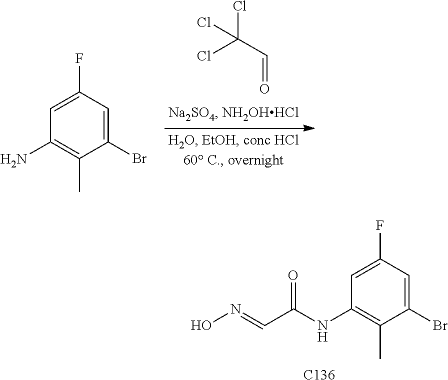

A mixture of sodium sulfate (111.38 g, 784.152 mmol, 8 equiv), hydroxylamine hydrochloride (23.84 g, 343.067 mmol, 3.5 equiv), and chloral (21.67 g, 147.029 mmol, 1.5 equiv) was dissolved in water (500 mL). To the reaction mixture was added a solution of 3-bromo-5-fluoro-2-methylaniline (20 g, 98.019 mmol, 1 equiv) in a mixture of water (540 mL), ethanol (70 mL), and concentrated HCl (17 mL). The reaction mixture was stirred overnight at 60° C., then cooled to room temperature. A precipitate formed that was collected by filtration and washed with water (2×50 mL). The resulting solid was dried in an oven under reduced pressure to afford (2E)-N-(3-bromo-5-fluoro-2-methylphenyl)-2-(N-hydroxyimino)acetamide (22 g, 82%) as a solid. LCMS (ES, m/z): 275[M+H]⁺.

### Synthesis of Intermediate C137

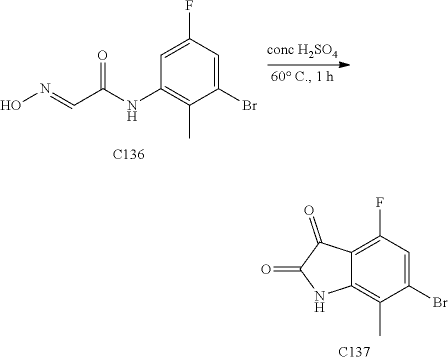

(2E)-N-(3-bromo-5-fluoro-2-methylphenyl)-2-(N-hydroxyimino)acetamide (22 g, 79.978 mmol, 1 equiv) was added to sulfuric acid (170 mL) in portions at 60° C. The resulting mixture was stirred for 1 h at 60° C., then cooled to room temperature and slowly added to ice water. A precipitate formed that was collected by filtration and washed with water (2×20 mL). The resulting solid was dried under vacuum to afford 6-bromo-4-fluoro-7-methyl-1H-indole-2,3-dione (18.5 g, 90%) as a solid. LCMS (ES, m/z): 258[M+H]⁺.

### Synthesis of Intermediate C138

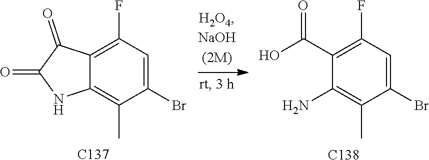

To a mixture of 6-bromo-4-fluoro-7-methyl-1H-indole-2,3-dione (18.5 g, 71.693 mmol, 1 equiv) and NaOH (2 M) (91 mL, 645.237 mmol, 9 equiv) was added H₂O₂ (15.4 mL, 358.465 mmol, 5 equiv) dropwise over 15 min at room temperature. The resulting mixture was stirred for an additional 3 h at room temperature, then quenched with saturated sodium sulfite (aq.) at room temperature and neutralized to pH 7 with HCl (2 M). The resulting mixture was filtered, and the filter cake washed with water (2×20 mL). The filtrate was concentrated under reduced pressure to give a residue. The residue was acidified to pH 4 with HCl (2 M). The precipitated solids were collected by filtration and washed with water (2×20 mL). The resulting solid was dried under infrared light to afford 2-amino-4-bromo-6-fluoro-3-methylbenzoic acid (16 g, 90%) as a solid. LCMS (ES, m/z): 249[M+H]⁺.

### Synthesis of Intermediate C139

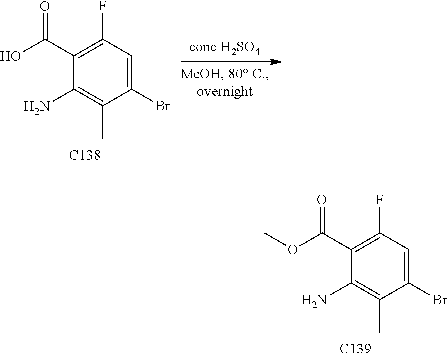

To a stirred solution of 2-amino-4-bromo-6-fluoro-3-methylbenzoic acid (6 g, 24.189 mmol, 1 equiv) in methanol (60 mL) was added sulfuric acid (23.72 g, 241.890 mmol, 10 equiv) dropwise at room temperature. The resulting mixture was stirred for 16 h at 80° C., then concentrated under reduced pressure to give a residue. The residue was quenched with a mixture of water and ice (50 mL) at room temperature. The resulting mixture was extracted with DCM (3×50 mL). The organic layers were combined, washed with brine (lx 50 mL), dried over anhydrous Na₂SO₄, and filtered. After filtration, the filtrate was concentrated under reduced pressure to give a residue. The residue was purified by silica gel column chromatography, eluted with PE/EA (1:1) to afford methyl 2-amino-4-bromo-6-fluoro-3-methylbenzoate (2.48 g, 39%) as an oil. LCMS (ES, m/z): 262[M+H]⁺.

### Synthesis of Intermediate C140

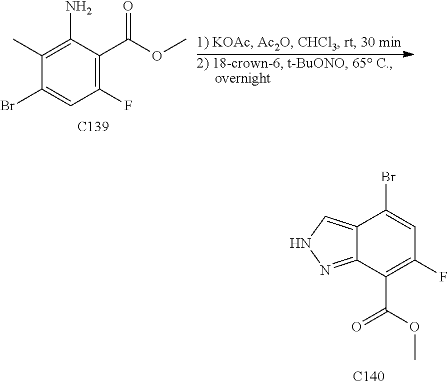

To a stirred solution of methyl 2-amino-4-bromo-6-fluoro-3-methylbenzoate (2.35 g, 8.967 mmol, 1 equiv) and KOAc (0.97 g, 9.864 mmol, 1.1 equiv) in CHCl₃ (50 mL) was added Ac₂0 (1.83 g, 17.934 mmol, 2 equiv) dropwise at 0° C. The resulting mixture was stirred for 20 min at room temperature. To the reaction mixture was added 18-Crown-6 (0.43 g, 1.614 mmol, 0.18 equiv) and tBuONO (2.03 g, 19.727 mmol, 2.2 equiv). The resulting mixture was stirred for an additional 2 h at 65° C. The resulting mixture was diluted with saturated NaHCO₃ (aq.) (20 mL) and extracted with DCM (2×50 mL). The organic layers were combined, washed with brine (30 mL), dried over anhydrous Na₂SO₄, and filtered. After filtration, the filtrate was concentrated under reduced pressure to give a residue. The residue was triturated with diethyl ether (10 mL) to afford methyl 4-bromo-6-fluoro-2H-indazole-7-carboxylate (1.8 g, 74%) as a solid. LCMS (ES, m/z): 273 M+H]⁺.

### Synthesis of Intermediate C141

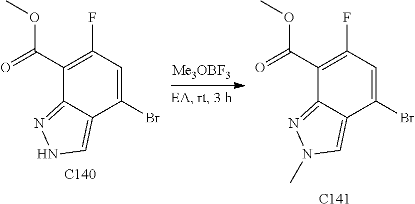 [SMILES: "C140" ; COC(=O)c1c(F)cc(Br)c2c[nH]nc12 ; COC(=O)c1c(F)cc(Br)c2cn(C)nc12 ; "C141"]

To a stirred solution of methyl 4-bromo-6-fluoro-2H-indazole-7-carboxylate (1.7 g, 6.226 mmol, 1 equiv) in ethyl acetate (50 mL) was added trimethyloxonium tetrafluoroborate (1.38 g, 9.339 mmol, 1.5 equiv) at room temperature. The resulting mixture was stirred for 3 h at room temperature, then diluted with ethyl acetate (50 mL) and washed with brine (3×50 mL). The organic phase was concentrated under reduced pressure to afford methyl 4-bromo-6-fluoro-2-methylindazole-7-carboxylate (1.7 g, 95%) as a solid. LCMS (ES, m/z): 287[M+H]⁺.

### Synthesis of Intermediate C142

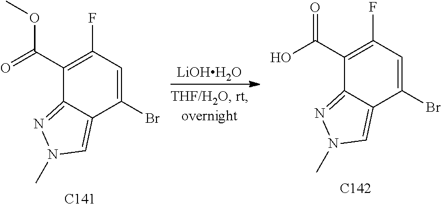

To a stirred mixture of methyl 4-bromo-6-fluoro-2-methylindazole-7-carboxylate (1.6 g, 5.573 mmol, 1 equiv) in THF (12 mL) and water (4 mL) was added lithiumol hydrate (0.47 g, 11.146 mmol, 2 equiv) at room temperature. The resulting mixture was stirred overnight at room temperature, then concentrated under reduced pressure, diluted with water (20 mL), and acidified to pH 3 with citric acid. A precipitate formed that was collected by filtration and washed with water (2×10 mL) to afford 4-bromo-6-fluoro-2-methylindazole-7-carboxylic acid (1.5 g, 99%) as a solid. LCMS (ES, m/z): 273[M+H]⁺.

### Synthesis of Intermediate C143

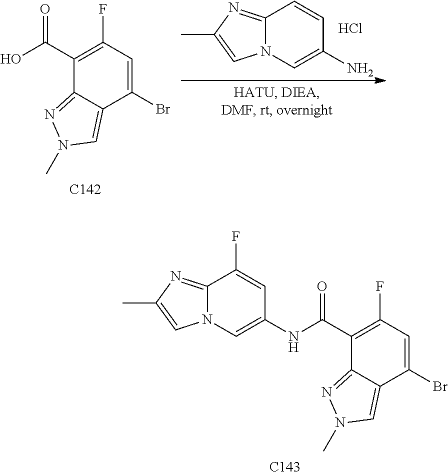 [SMILES: Cn1cc2c(Br)cc(F)c(C(=O)O)c2n1 ; Cc1cn2cc(N)ccc2n1 ; Cl ; "C142" ; Cc1cn2cc(NC(=O)c3c(F)cc(Br)c4cn(C)nc34)cc(F)c2n1 ; "C143"]

To a stirred mixture of 4-bromo-6-fluoro-2-methylindazole-7-carboxylic acid (500 mg, 1.831 mmol, 1 equiv) and HATU (835.49 mg, 2.197 mmol, 1.2 equiv) in DMF (15 mL) was added DIEA (946.65 mg, 7.324 mmol, 4 equiv) and 8-fluoro-2-methylimidazo[1,2-a]pyridin-6-amine hydrochloride (369.20 mg, 1.831 mmol, 1 equiv) at room temperature. The resulting mixture was stirred overnight at room temperature, then diluted with tert-butyl methyl ether (40 mL). A precipitate formed that was collected by filtration and washed with tert-butyl methyl ether (2×10 mL) to afford 4-bromo-6-fluoro-N-{8-fluoro-2-methylimidazo[1,2-a]pyridin-6-yl}-2-methylindazole-7-carboxamide (530 mg, 69%) as a solid. LCMS (ES, m/z): 420[M+H]⁺.

### Synthesis of Intermediate C144

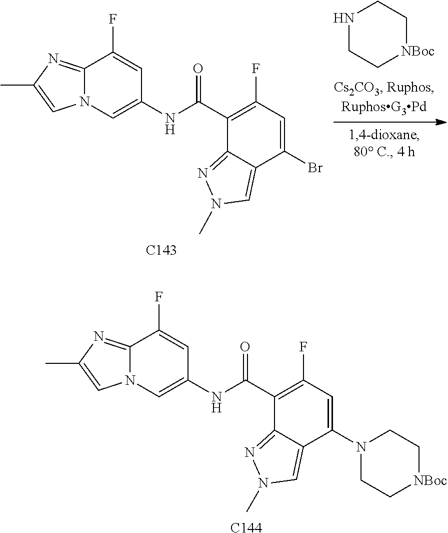 [SMILES: Cc1cn2cc(NC(=O)c3c(F)cc(Br)c4cn(C)nc34)cc(F)c2n1 ; CC(C)(C)OC(=O)N1CCNCC1 ; "C143" ; Cc1cn2cc(NC(=O)c3c(F)cc(N4CCN(C(=O)OC(C)(C)C)CC4)c4cn(C)nc34)cc(F)c2n1 ; "C144"]

To a mixture of 4-bromo-6-fluoro-N-{8-fluoro-2-methylimidazo[1,2-a]pyridin-6-yl}-2-methylindazole-7-carboxamide (200 mg, 0.476 mmol, 1 equiv) and tert-butyl piperazine-1-carboxylate (177.29 mg, 0.952 mmol, 2 equiv) in 1,4-dioxane (6 mL) was added Cs₂CO₃ (465.21 mg, 1.428 mmol, 3 equiv), Ruphos (44.42 mg, 0.095 mmol, 0.2 equiv), and RuPhos Palladacycle Gen.3 (39.81 mg, 0.048 mmol, 0.1 equiv). The reaction mixture was stirred for 4 h at 80° C. under nitrogen atmosphere, then concentrated under reduced pressure to give a residue. The residue was purified by silica gel column chromatography, eluted with PE/EA (1:1) to afford tert-butyl 4-[6-fluoro-7-({8-fluoro-2-methylimidazo[1,2-a]pyridin-6-yl}carbamoyl)-2-methylindazol-4-yl]piperazine-1-carboxylate (230 mg, 92%) as a solid. LCMS (ES, m/z): 526[M+H]⁺.

### Synthesis of Compound 360

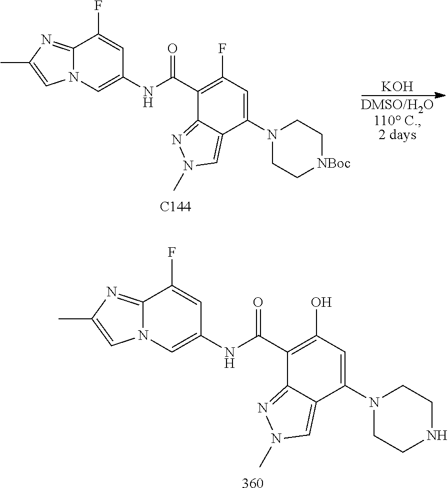

To a stirred solution of tert-butyl 4-{6-fluoro-7-[({8-fluoro-2-methyl-octahydroimidazo[1,2-a]pyridin-6-yl}amino)(hydroxy)methyl]-2-methyl-octahydroindazol-4-yl}piperazine-1-carboxylate (70 mg, 0.129 mmol, 1 equiv) in DMSO (28 mL) and water (0.7 mL) was added potassium hydroxide (72.23 mg, 1.290 mmol, 10 equiv). The resulting mixture was stirred for 2 days at 110° C. The product was purified by Prep-HPLC (Condition 10, Gradient 4) to afford 7-[({8-fluoro-2-methyl-octahydroimidazo[1,2-a]pyridin-6-yl}amino)(hydroxy)methyl]-2-methyl-4-(piperazin-1-yl)-octahydroindazol-6-ol (3.1 mg, 5%) as a solid. LCMS (ES, m/z): 424 [M+H]⁺. ¹H NMR (300 MHz, Methanol-d₄) δ 9.37 (s, 1H), 8.43 (s, 1H), 7.99 (s, 1H), 7.82 (d, J=11.4 Hz, 1H), 6.17 (s, 1H), 4.24 (s, 3H), 3.64 (d, J=5.5 Hz, 4H), 3.47 (d, J=5.4 Hz, 4H), 2.54 (s, 3H).

### Example 108: Synthesis of Compound 348

### Synthesis of Intermediate C145

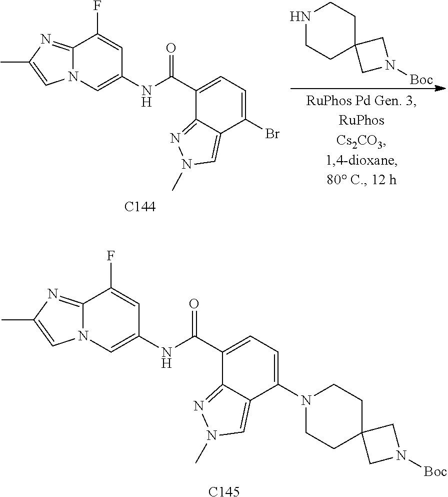 [SMILES: Cc1cn2cc(NC(=O)c3ccc(Br)c4cn(C)nc34)cc(F)c2n1 ; CC(C)(C)OC(=O)N1CC2(CCNCC2)C1 ; "C144" ; Cc1cn2cc(NC(=O)c3ccc(N4CCC5(CC4)CN(C(=O)OC(C)(C)C)C5)c4cn(C)nc34)cc(F)c2n1 ; "C145"]

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl 2,7-diazaspiro[3.5]nonane-2-carboxylate (40.51 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford 4-{2,7-diazaspiro[3.5]nonan-7-yl}-N-{8-fluoro-2-methylimidazo[1,2-a]pyridin-6-yl}-2-methylindazole-7-carboxamide (60 mg, 81%) as a solid. LCMS (ES, m/z): 548[M+H]⁺.

### Synthesis of Compound 348

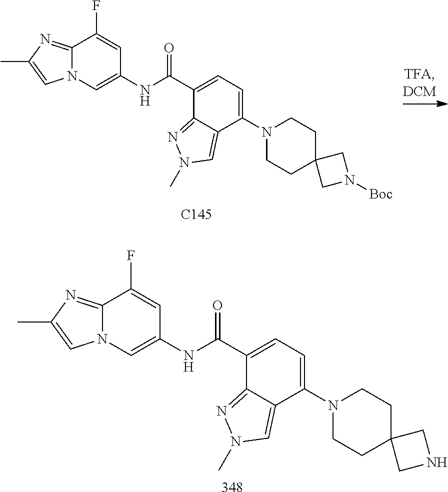

To a stirred solution of tert-butyl 7-[7-({8-fluoro-2-methylimidazo[1,2-a]pyridin-6-yl}carbamoyl)-2-methylindazol-4-yl]-2,7-diazaspiro[3.5]nonane-2-carboxylate (60 mg, 0.110 mmol, 1 equiv) in DCM (1 mL) was added TFA (1 mL, 13.463 mmol, 122.88 equiv) at room temperature. The resulting mixture was stirred for 1 h at room temperature, then concentrated under reduced pressure to give a residue. The residue was purified by Prep-HPLC (Condition 5, Gradient 1) to afford 4-{2,7-diazaspiro[3.5]nonan-7-yl}-N-{8-fluoro-2-methylimidazo[1,2-a]pyridin-6-yl}-2-methylindazole-7-carboxamide (22.1 mg, 45%) as a solid. LCMS (ES, m/z): 448 [M+H]⁺. ¹H NMR (400 MHz, DMSO-d₆) δ 11.06 (s, 1H), 9.21 (d, J=1.7 Hz, 1H), 8.78 (s, 1H), 7.97 (d, J=8.1 Hz, 1H), 7.90 (d, J=3.1 Hz, 1H), 7.34 (dd, J=12.4, 1.7 Hz, 1H), 6.50 (d, J=8.3 Hz, 1H), 4.30 (s, 3H), 3.63 (s, 2H), 3.40-3.35 (m, 6H), 2.35 (s, 3H), 1.89 (t, J=5.5 Hz, 4H).

### Example 109: Synthesis of Compound 349

### Synthesis of Intermediate C146

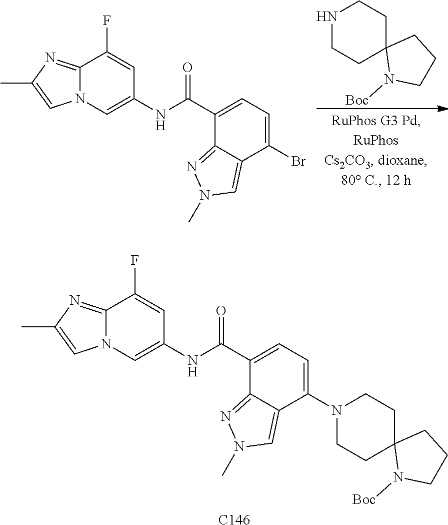 [SMILES: Cc1cn2cc(NC(=O)c3ccc(Br)c4cn(C)nc34)cc(F)c2n1 ; CC(C)(C)OC(=O)N1CCCC12CCNCC2 ; Cc1cn2cc(NC(=O)c3ccc(N4CCC5(CCCN5C(=O)OC(C)(C)C)CC4)c4cn(C)nc34)cc(F)c2n1 ; "C146"]

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl 1,8-diazaspiro[4.5]decane-1-carboxylate (43.02 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl 8-[7-({8-fluoro-2-methylimidazo[1,2-a]pyridin-6-yl}carbamoyl)-2-methylindazol-4-yl]-1,8-diazaspiro[4.5]decane-1-carboxylate (70 mg, 84%) as a solid. LCMS (ES, m/z): 562[M+H]⁺.

### Synthesis of Compound 349

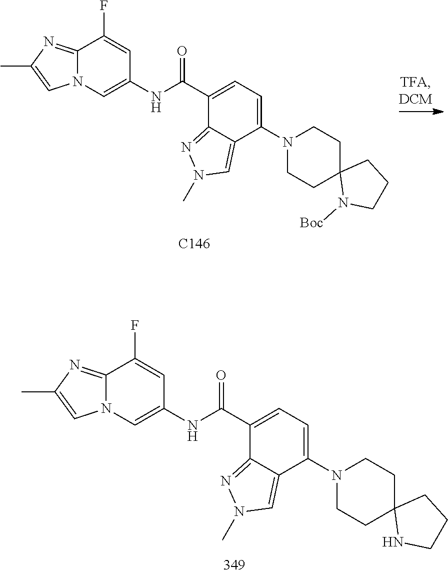

To a stirred solution of tert-butyl 8-[7-({8-fluoro-2-methylimidazo[1,2-a]pyridin-6-yl}carbamoyl)-2-methylindazol-4-yl]-1,8-diazaspiro[4.5]decane-1-carboxylate (70 mg, 0.125 mmol, 1 equiv) in DCM (1 mL) was added TFA (1 mL, 13.463 mmol, 108.02 equiv) at room temperature. The resulting mixture was stirred for 1 h at room temperature, then concentrated under reduced pressure to give a residue. The residue was purified by Prep-HPLC (Condition 5, Gradient 1) to afford 4-{1,8-diazaspiro[4.5]decan-8-yl}-N-{8-fluoro-2-methylimidazo[1,2-a]pyridin-6-yl}-2-methylindazole-7-carboxamide (23.8 mg, 41%) as a solid. LCMS (ES, m/z): 462 [M+H]⁺. ¹H NMR (400 MHz, DMSO-d₆) δ 11.06 (s, 1H), 9.21 (s, 1H), 8.77 (s, 1H), 7.96 (d, J=7.9 Hz, 1H), 7.90 (d, J=3.1 Hz, 1H), 7.33 (d, J=12.2 Hz, 1H), 6.49 (d, J=7.9 Hz, 1H), 4.29 (s, 3H), 3.59-3.42 (m, 4H), 2.91-2.83 (m, 3H), 1.76-1.65 (m, 6H), 1.56 (t, J=7.4 Hz, 2H).

### Example 110: Synthesis of Compound 361

### Synthesis of Compound 361

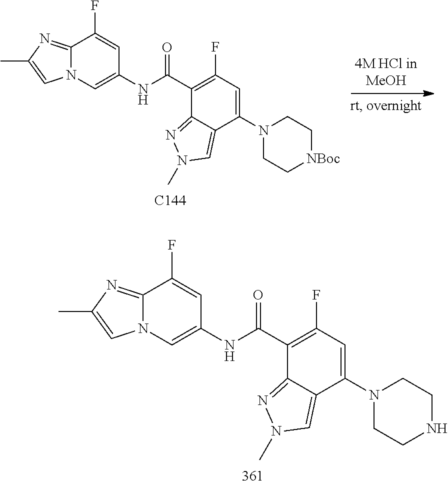

To a stirred solution of tert-butyl 4-[6-fluoro-7-({8-fluoro-2-methylimidazo[1,2-a]pyridin-6-yl}carbamoyl)-2-methylindazol-4-yl]piperazine-1-carboxylate (50 mg, 0.095 mmol, 1 equiv) in methanol (1 mL) was added HCl (g) in methanol (1 mL, 32.913 mmol, 345.95 equiv) at room temperature. The resulting mixture was stirred for 1 h at room temperature, then concentrated under reduced pressure to give a residue. The residue was purified by Prep-HPLC with the following conditions (Condition 5, Gradient 1) to afford 6-fluoro-N-{8-fluoro-2-methylimidazo[1,2-a]pyridin-6-yl}-2-methyl-4-(piperazin-1-yl) indazole-7-carboxamide (18.7 mg, 46%) as a solid. LCMS (ES, m/z): 426 [M+H]⁺. ¹H NMR (400 MHz, DMSO-d₆) δ 10.90 (s, 1H), 9.19 (d, J=1.6 Hz, 1H), 8.77 (s, 1H), 7.90 (d, J=3.1 Hz, 1H), 7.28-7.21 (m, 1H), 6.24 (d, J=15.0 Hz, 1H), 4.20 (s, 3H), 3.31 (s, 4H), 2.90 (s, 4H), 2.35 (s, 3H).

### Example 111: Synthesis of Compound 362

### Synthesis of Intermediate C147

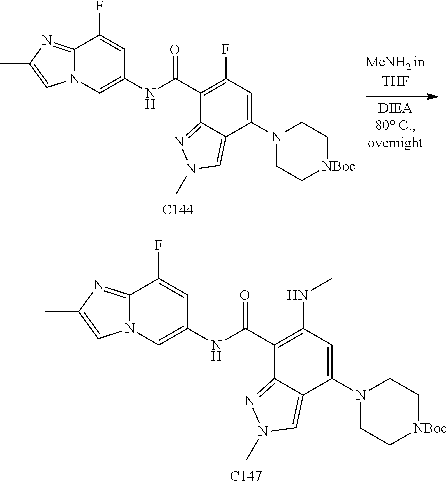 [SMILES: Cc1cn2cc(NC(=O)c3c(F)cc(N4CCN(C(=O)OC(C)(C)C)CC4)c4cn(C)nc34)cc(F)c2n1 ; "C144" ; CNc1cc(N2CCN(C(=O)OC(C)(C)C)CC2)c2cn(C)nc2c1C(=O)Nc1cc(F)c2nc(C)cn2c1 ; "C147"]

A solution of tert-butyl 4-[6-fluoro-7-({8-fluoro-2-methylimidazo[1,2-a]pyridin-6-yl}carbamoyl)-2-methylindazol-4-yl]piperazine-1-carboxylate (60 mg, 0.114 mmol, 1 equiv) and DIEA (29.51 mg, 0.228 mmol, 2 equiv) in Methylamine (2M in THF, 5 mL) was stirred overnight at 80° C. The resulting mixture was concentrated under reduced pressure to give a residue. The residue was purified by silica gel column chromatography, eluted with PE/EA (1:1) to afford tert-butyl 4-[7-({8-fluoro-2-methylimidazo[1,2-a]pyridin-6-yl}carbamoyl)-2-methyl-6-(methylamino) indazol-4-yl]piperazine-1-carboxylate (60 mg, 98%) as a solid. LCMS (ES, m/z): 537[M+H]⁺.

### Synthesis of Compound 362

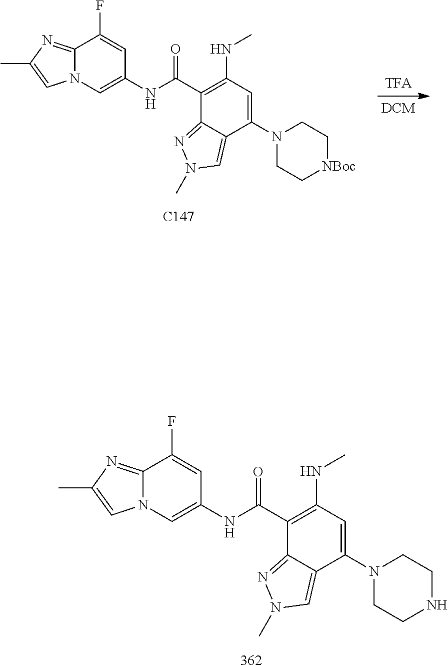

To a stirred solution of tert-butyl 4-[7-({8-fluoro-2-methylimidazo[1,2-a]pyridin-6-yl}carbamoyl)-2-methyl-6-(methylamino) indazol-4-yl]piperazine-1-carboxylate (60 mg, 0.112 mmol, 1 equiv) in DCM (1 mL) was added TFA (1 mL, 10.202 mmol, 91.24 equiv) at room temperature. The resulting mixture was stirred for 1 h at room temperature, then concentrated under reduced pressure to give a residue. The residue was purified by Prep-HPLC (Condition 10, Gradient 4) to afford N-{8-fluoro-2-methylimidazo[1,2-a]pyridin-6-yl}-2-methyl-6-(methylamino)-4-(piperazin-1-yl) indazole-7-carboxamide (27.2 mg, 56%) as a solid. LCMS (ES, m/z): 437 [M+H]⁺. ¹H NMR (400 MHz, DMSO-d6) δ 11.58 (s, 1H), 9.56 (q, J=4.9 Hz, 1H), 9.10 (d, J=1.6 Hz, 1H), 8.52 (s, 1H), 7.85 (d, J=3.4 Hz, 1H), 7.24 (dd, J=12.5, 1.7 Hz, 1H), 5.90 (s, 1H), 4.15 (s, 3H), 3.45 (t, J=5.0 Hz, 4H), 3.14 (d, J=6.0 Hz, 4H), 2.98 (d, J=5.0 Hz, 3H), 2.34 (s, 3H).

### Example 112: Synthesis of Compound 363

### Synthesis of Intermediate C148

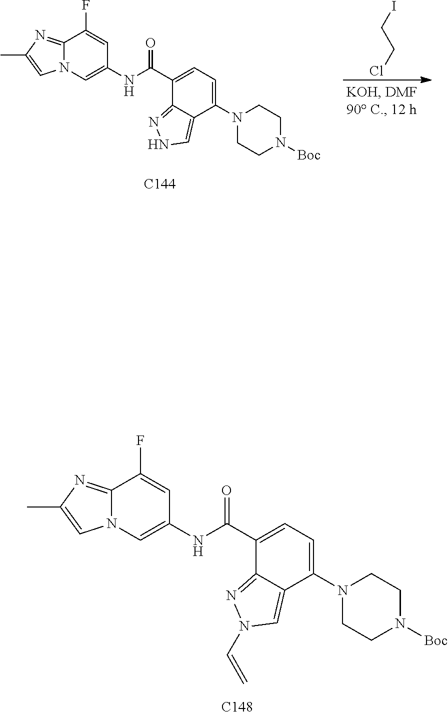

A mixture of tert-butyl 4-[7-({8-fluoro-2-methylimidazo[1,2-a]pyridin-6-yl}carbamoyl)-2H-indazol-4-yl]piperazine-1-carboxylate (100 mg, 0.203 mmol, 1 equiv), 1-chloro-2-iodoethane (57.87 mg, 0.304 mmol, 1.5 equiv), and KOH (68.21 mg, 1.218 mmol, 6.0 equiv) in DMF (2 mL) was stirred for 12 h at 90° C. The mixture was purified by reverse flash chromatography (Condition 2, Gradient 2) to afford tert-butyl 4-[2-ethenyl-7-({8-fluoro-2-methylimidazo[1,2-a]pyridin-6-yl}carbamoyl) indazol-4-yl]piperazine-1-carboxylate (100 mg) as a solid. LCMS (ES, m/z): 520 [M+H]⁺.

### Synthesis of Compound 363

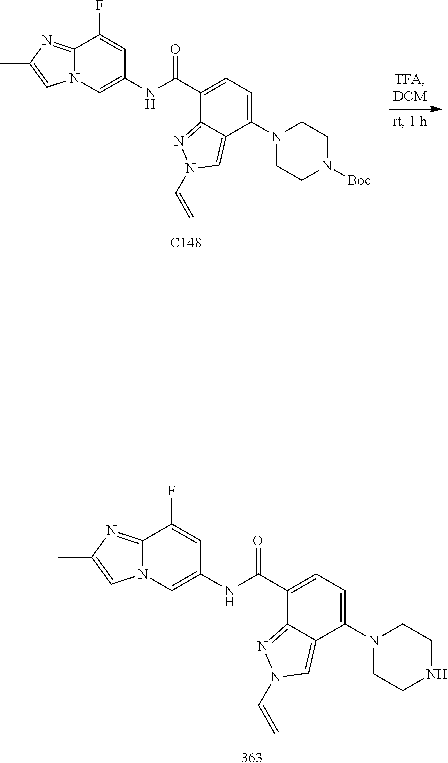

A mixture of tert-butyl 4-[2-ethenyl-7-({8-fluoro-2-methylimidazo[1,2-a]pyridin-6-yl}carbamoyl) indazol-4-yl]piperazine-1-carboxylate (100 mg, 0.192 mmol, 1 equiv) and TFA (0.3 mL) in DCM (2 mL) was stirred for 1 h at room temperature. The resulting mixture was basified to pH 8 with 7 M NH₃(gas) in methanol, then concentrated under reduced pressure to give a residue. The residue was purified by reverse flash chromatography (Condition 1, Gradient 5) to afford 2-ethenyl-N-{8-fluoro-2-methylimidazo[1,2-a]pyridin-6-yl}-4-(piperazin-1-yl) indazole-7-carboxamide (24 mg, 30%) as a solid. LCMS (ES, m/z): 420 [M+H]⁺. ¹H NMR (300 MHz, DMSO-d₆) δ 10.91 (s, 1H), 9.22 (d, J=1.7 Hz, 1H), 9.10 (s, 1H), 8.04 (d, J=8.1 Hz, 1H), 7.95-7.87 (m, 1H), 7.67 (dd, J=15.5, 8.8 Hz, 1H), 7.35 (dd, J=12.3, 1.6 Hz, 1H), 6.52 (d, J=8.2 Hz, 1H), 6.26 (d, J=15.3 Hz, 1H), 5.36 (d, J=8.6 Hz, 1H), 3.39 (d, J=5.3 Hz, 4H), 2.94 (s, 4H), 2.39-2.32 (m, 3H).

### Example 113: Synthesis of Compounds 352 and 353

### Synthesis of Compounds 352 and 353

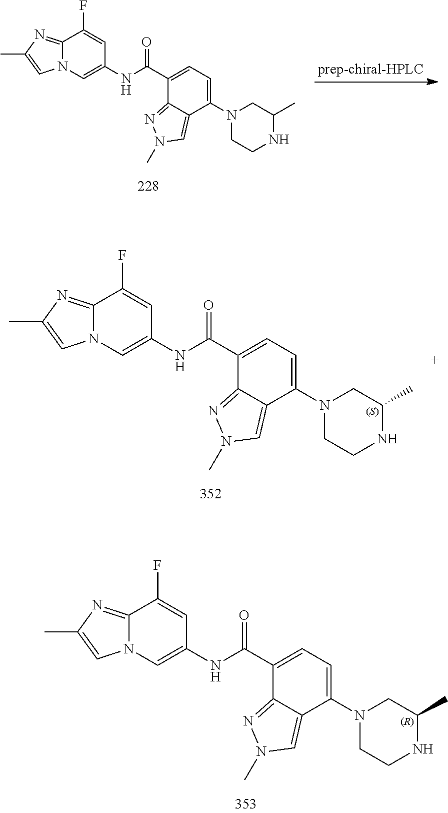

Compounds 352 and 353 were isolated by Chiral Prep-HPLC (Condition 3, Gradient 1) Compound 352: RT=4.680 min. LCMS (ES, m/z): 422 [M+H]⁺. ¹H NMR (400 MHz, Methanol-d₄) δ 9.06 (d, J=1.8 Hz, 1H), 8.49 (d, J=2.0 Hz, 1H), 8.08 (dd, J=8.2, 1.1 Hz, 1H), 7.70 (t, J=2.2 Hz, 1H), 7.22 (dq, J=11.8, 1.7 Hz, 1H), 6.53 (dd, J=8.2, 1.5 Hz, 1H), 4.32 (s, 3H), 3.82 (d, J=11.7 Hz, 2H), 3.20-2.93 (m, 4H), 2.66 (dd, J=12.3, 10.3 Hz, 1H), 2.43 (s, 3H), 1.21 (d, J=6.4 Hz, 3H). Compound 353: RT=5.317 min. LCMS (ES, m/z): 422 [M+H]⁺. ¹H NMR (400 MHz, Methanol-d₄) δ 9.04 (s, 1H), 8.47 (d, J=3.8 Hz, 1H), 8.06 (dd, J=8.9, 1.8 Hz, 1H), 7.68 (d, J=3.3 Hz, 1H), 7.22-7.18 (m, 1H), 6.51 (dd, J=8.2, 3.0 Hz, 1H), 4.31 (d, J=1.6 Hz, 3H), 3.81 (dd, J=11.0, 5.2 Hz, 2H), 3.15-2.96 (m, 4H), 2.65 (dd, J=12.5, 10.3 Hz, 1H), 2.43 (d, J=1.0 Hz, 3H), 1.21 (d, J=6.4 Hz, 3H).

### Example 114: Synthesis of Compound 364

### Synthesis of Intermediate C149

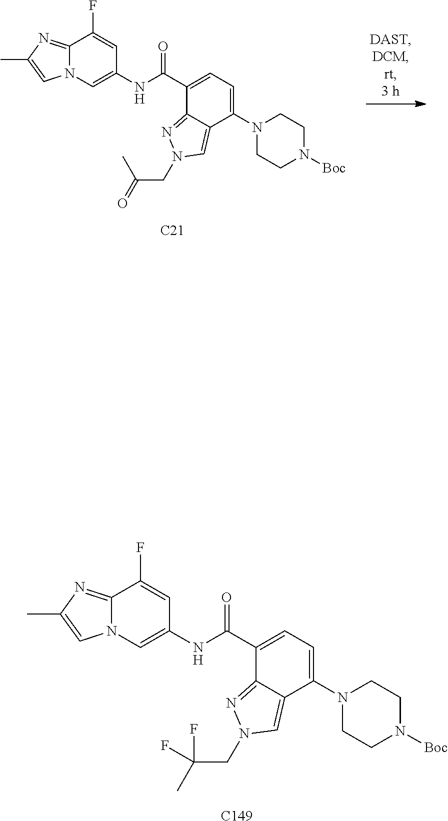

A mixture of tert-butyl 4-[7-({8-fluoro-2-methylimidazo[1,2-a]pyridin-6-yl}carbamoyl)-2-(2-oxopropyl) indazol-4-yl]piperazine-1-carboxylate (60.0 mg, 0.10 mmol, 1.0 equiv) in DCM (2 mL) was treated with DAST (35.1 mg, 0.21 mmol, 2.0 equiv) at 0° C. The resulting mixture was stirred for 3 h at room temperature, then quenched with ice-water at 0° C. The resulting mixture was extracted with ethyl acetate (3×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with EA to afford tert-butyl 4-[2-(2,2-difluoropropyl)-7-({8-fluoro-2-methylimidazo[1,2-a]pyridin-6-yl}carbamoyl) indazol-4-yl]piperazine-1-carboxylate (29 mg, 46%) as a solid. LCMS (ES, m/z): 572 [M+H]⁺.

### Synthesis of Compound 364

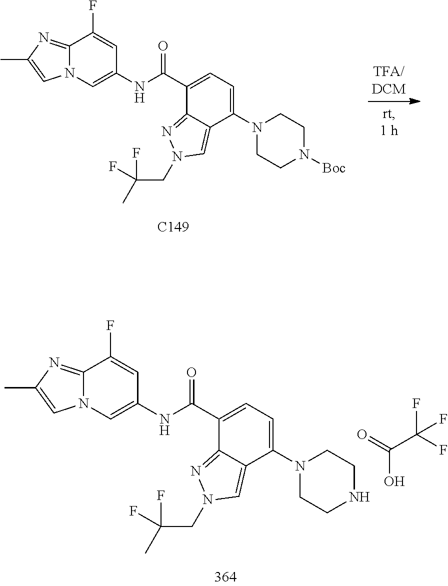

A solution of tert-butyl 4-[2-(2,2-difluoropropyl)-7-({8-fluoro-2-methylimidazo[1,2-a]pyridin-6-yl}carbamoyl) indazol-4-yl]piperazine-1-carboxylate (29.0 mg, 0.05 mmol, 1.0 equiv) in DCM (0.5 mL) was treated with TFA (0.5 mL) at room temperature. The resulting mixture was stirred for 1 h at room temperature, then concentrated under reduced pressure to give a residue. The residue was purified by Prep-HPLC (Condition 3, Gradient 1) to afford 2-(2,2-difluoropropyl)-N-{8-fluoro-2-methylimidazo[1,2-a]pyridin-6-yl}-4-(piperazin-1-yl) indazole-7-carboxamide; trifluoroacetic acid (8 mg, 27%) as a solid. LCMS (ES, m/z): 472 [M+H]⁺. ¹H NMR (400 MHz, DMSO-d₆) δ 11.19 (s, 1H), 9.40 (s, 1H), 9.00 (s, 1H), 8.86-8.85 (m, 2H), 8.08-8.06 (m, 2H), 7.53 (d, J=11.9 Hz, 1H), 6.66 (d, J=8.0 Hz, 1H), 5.17 (t, J=13.1 Hz, 2H), 3.60-3.59 (m, 4H), 3.37-3.35 (m, 4H), 2.41 (s, 3H), 1.78 (t, J=19.2 Hz, 3H).

### Example 115: Synthesis of Compound 365

### Synthesis of Intermediate C150

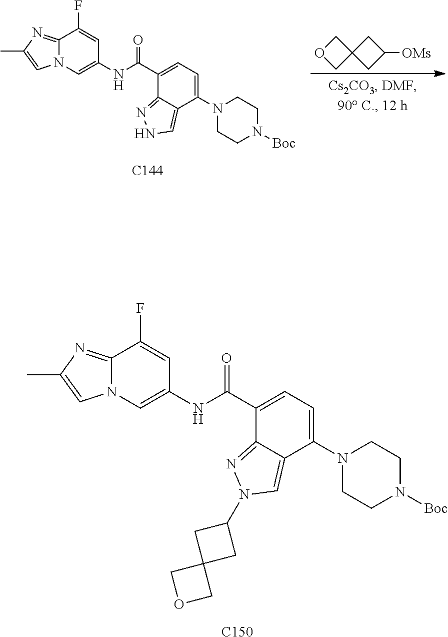

A solution of tert-butyl 4-[7-({8-fluoro-2-methylimidazo[1,2-a]pyridin-6-yl}carbamoyl)-2H-indazol-4-yl]piperazine-1-carboxylate (50 mg, 0.1 mmol, 1 equiv), 2-oxaspiro[3.3]heptan-6-yl methanesulfonate (50 mg, 0.26 mmol, 2.57 equiv), and Cs₂CO₃ (99 mg, 0.3 mmol, 3.0 equiv) in DMF (2 mL) was stirred for 12 h at 90° C. The reaction mixture was purified by reverse flash chromatography (Condition 2, Gradient 3) to afford tert-butyl 4-[7-({8-fluoro-2-methylimidazo[1,2-a]pyridin-6-yl}carbamoyl)-2-{2-oxaspiro[3.3]heptan-6-yl}indazol-4-yl]piperazine-1-carboxylate (50 mg, 84%) as a solid. LCMS (ES, m/z): 590 [M+H]⁺.

### Synthesis of Compound 365

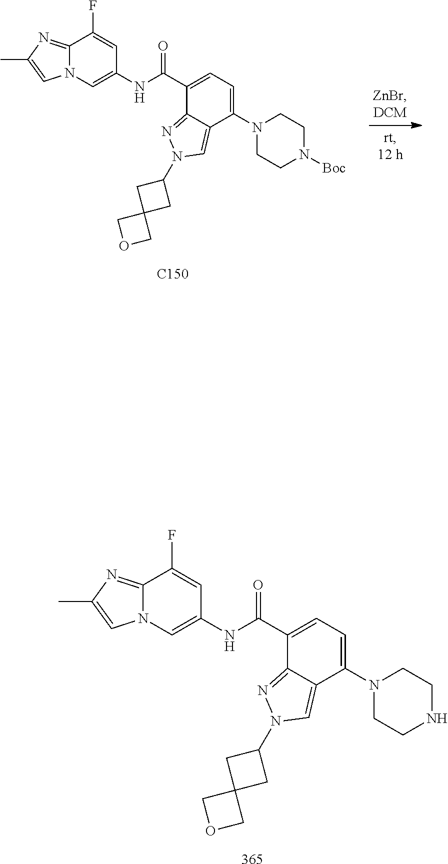

A mixture of tert-butyl 4-[7-({8-fluoro-2-methylimidazo[1,2-a]pyridin-6-yl}carbamoyl)-2-{2-oxaspiro[3.3]heptan-6-yl}indazol-4-yl]piperazine-1-carboxylate (30 mg, 0.05 mmol, 1 equiv) and ZnBr₂ (30 mg, 0.133 mmol, 2.62 equiv) in DCM (1 mL) was stirred for 12 h at room temperature. The resulting mixture was concentrated under reduced pressure to give a residue. The residue was purified by reverse flash chromatography (Condition 1, Gradient 5) to afford N-{8-fluoro-2-methylimidazo[1,2-a]pyridin-6-yl}-2-{2-oxaspiro[3.3]heptan-6-yl}-4-(piperazin-1-yl) indazole-7-carboxamide (2 mg, 8%) as a solid. LCMS (ES, m/z): 490 [M+H]⁺. ¹H NMR (300 MHz, DMSO-d₆) δ 11.01 (s, 1H), 9.20 (d, J=1.7 Hz, 1H), 8.84 (s, 1H), 7.98 (d, J=8.1 Hz, 1H), 7.92 (d, J=3.1 Hz, 1H), 7.22 (dd, J=12.3, 1.7 Hz, 1H), 6.49 (d, J=8.2 Hz, 1H), 5.19 (p, J=8.1 Hz, 1H), 4.77 (s, 2H), 4.70 (s, 2H), 3.32-3.26 (m, 4H), 2.96-2.91 (m, 8H), 2.39-2.33 (m, 3H).

### Example 116: Synthesis of Compound 366

### Synthesis of Intermediate C151

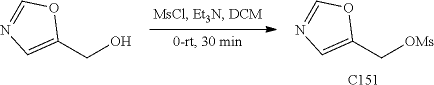 [SMILES: OCc1cnco1 ; CS(=O)(=O)OCc1cnco1 ; "C151"]

To a stirred solution of 1,3-oxazol-5-ylmethanol (600.0 mg, 6.055 mmol, 1.0 equiv) in DCM (12 mL) was added Et₃N (919.1 mg, 9.082 mmol, 1.5 equiv) and MsCl (762.9 mg, 6.660 mmol, 1.1 equiv) dropwise at 0° C. under nitrogen atmosphere. The resulting mixture was stirred for 30 min at room temperature under nitrogen atmosphere, then concentrated under vacuum to give a residue.

### Synthesis of Intermediate C152

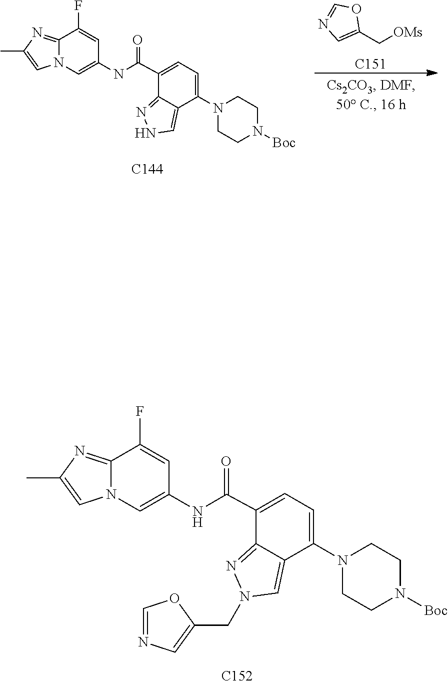

To a stirred mixture of tert-butyl 4-[7-({8-fluoro-2-methylimidazo[1,2-a]pyridin-6-yl}carbamoyl)-2H-indazol-4-yl]piperazine-1-carboxylate (600.0 mg, 1.216 mmol, 1.0 equiv) in DMF (12 mL) was added Cs₂CO₃ (1.18 g, 3.648 mmol, 3.0 equiv) and 1,3-oxazol-5-ylmethyl methanesulfonate (215.4 mg, 1.216 mmol, 1.0 equiv) at room temperature. The resulting mixture was stirred for 16 h at 50° C., then cooled to room temperature. The resulting mixture was diluted with water (20 mL) and extracted with ethyl acetate (3×20 mL). The organic layers were combined, washed with water (3×20 mL), dried over anhydrous Na₂SO₄, and filtered. After filtration, the filtrate was concentrated under reduced pressure to give a residue. The residue was purified by silica gel column chromatography, eluted with ethyl acetate to afford tert-butyl 4-[7-({8-fluoro-2-methylimidazo[1,2-a]pyridin-6-yl}carbamoyl)-2-(1,3-oxazol-5-ylmethyl) indazol-4-yl]piperazine-1-carboxylate (80 mg, 11%) as a solid. LCMS (ES, m/z): 575 [M+H]⁺.

### Synthesis of Compound 366

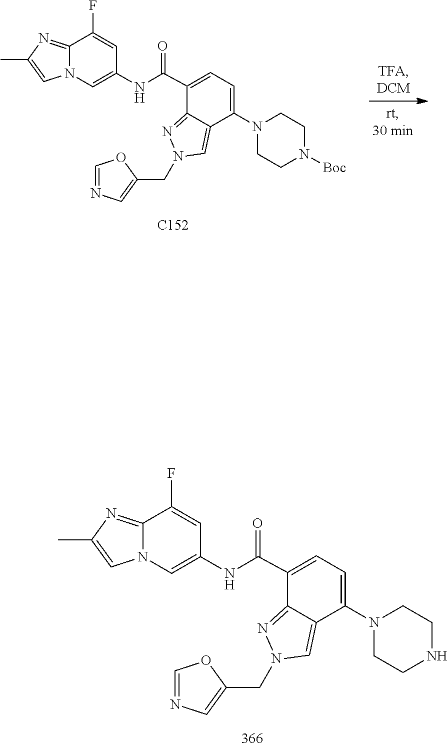 [SMILES: Cc1cn2cc(NC(=O)c3ccc(N4CCN(C(=O)OC(C)(C)C)CC4)c4cn(Cc5cnco5)nc34)cc(F)c2n1 ; "C152" ; Cc1cn2cc(NC(=O)c3ccc(N4CCNCC4)c4cn(Cc5cnco5)nc34)cc(F)c2n1 ; "366"]

To a stirred mixture of tert-butyl 4-[7-({8-fluoro-2-methylimidazo[1,2-a]pyridin-6-yl}carbamoyl)-2-(1,3-oxazol-5-ylmethyl) indazol-4-yl]piperazine-1-carboxylate (75.0 mg, 0.131 mmol, 1.0 equiv) in DCM (2 mL) was added TFA (0.2 mL) dropwise at 0° C. The resulting mixture was stirred for 30 min at room temperature, then concentrated under vacuum to give a residue. The residue was purified by Prep-HPLC (Condition 2, Gradient 8) to afford N-{8-fluoro-2-methylimidazo[1,2-a]pyridin-6-yl}-2-(1,3-oxazol-5-ylmethyl)-4-(piperazin-1-yl) indazole-7-carboxamide (20.2 mg, 33%) as a solid. LCMS (ES, m/z): 475 [M+H]⁺. ¹H NMR (400 MHz, DMSO-d₆) δ 11.02 (s, 1H), 9.21 (d, J=1.7 Hz, 1H), 8.95 (s, 1H), 8.42 (s, 1H), 7.99 (d, J=8.1 Hz, 1H), 7.92 (d, J 3.1 Hz, 1H), 7.50 (s, 1H), 7.14 (dd, J=12.2, 1.7 Hz, 1H), 6.51 (d, J=8.2 Hz, 1H), 5.95 (s, 2H), 3.38 (t, 4H), 2.93 (t, 4H), 2.36 (s, 3H).

### Example 117: Synthesis of Compound 367

### Synthesis of Intermediate C153

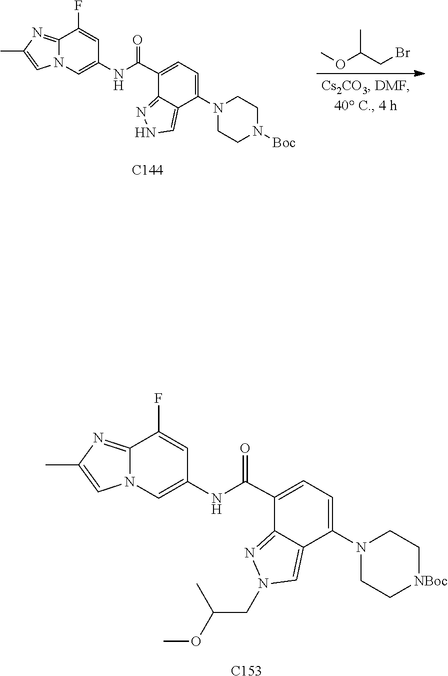

To a stirred mixture of tert-butyl 4-[7-({8-fluoro-2-methylimidazo[1,2-a]pyridin-6-yl}carbamoyl)-2H-indazol-4-yl]piperazine-1-carboxylate (120.0 mg, 0.24 mmol, 1.0 equiv) and 1-bromo-2-methoxypropane (55.8 mg, 0.36 mmol, 1.5 equiv) in DMF (1.5 mL) was added Cs₂CO₃ (237.6 mg, 0.73 mmol, 3.0 equiv) at room temperature. The resulting mixture was stirred for 4 h at 40° C., then diluted with water and extracted with ethyl acetate (3×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with EA to afford tert-butyl 4-[7-({8-fluoro-2-methylimidazo[1,2-a]pyridin-6-yl}carbamoyl)-2-(2-methoxypropyl) indazol-4-yl]piperazine-1-carboxylate (70 mg, 51%) as a solid. LCMS (ES, m/z): 566 [M+H]⁺.

### Synthesis of Compound 367

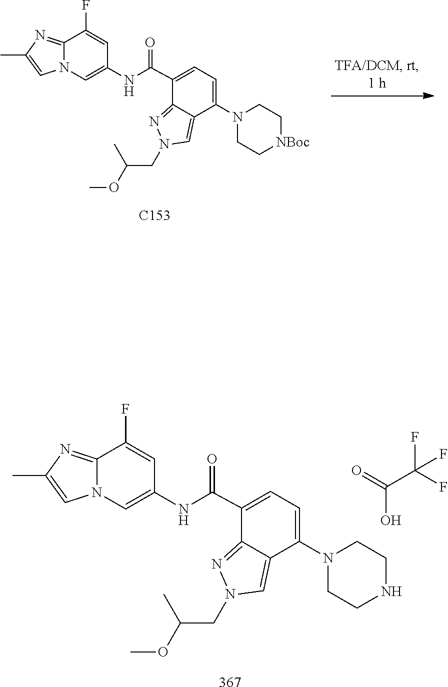

A solution of tert-butyl 4-[7-({8-fluoro-2-methylimidazo[1,2-a]pyridin-6-yl}carbamoyl)-2-(2-methoxypropyl) indazol-4-yl]piperazine-1-carboxylate (70.0 mg, 0.12 mmol, 1.0 equiv) in DCM (0.7 mL) was treated with TFA (0.7 mL) at room temperature. The resulting mixture was stirred for 1 h at room temperature, then concentrated under reduced pressure to give a residue. The residue was purified by Prep-HPLC (Condition 3, Gradient 15) to afford N-{8-fluoro-2-methylimidazo[1,2-a]pyridin-6-yl}-2-(2-methoxypropyl)-4-(piperazin-1-yl) indazole-7-carboxamide; trifluoroacetic acid (28 mg, 39%) as a solid. LCMS (ES, m/z): 466 [M+H]⁺. ¹H NMR (400 MHz, DMSO-d₆) δ 11.24 (s, 1H), 9.41 (d, J=1.6 Hz, 1H), 8.90-8.88 (m, 3H), 8.08 (d, J=2.7 Hz, 1H), 8.05 (d, J=8.0 Hz, 1H), 7.62 (d, J=11.9 Hz, 1H), 6.63 (d, J=8.1 Hz, 1H), 4.76-4.50 (m, 2H), 4.03 (td, J=6.8, 4.5 Hz, 1H), 3.60 (t, J=5.0 Hz, 4H), 3.37-3.35 (m, 4H), 3.24 (s, 3H), 2.42 (s, 3H), 1.21 (d, J=6.2 Hz, 3H).

### Example 118: Synthesis of Compound 368

### Synthesis of Intermediate C154

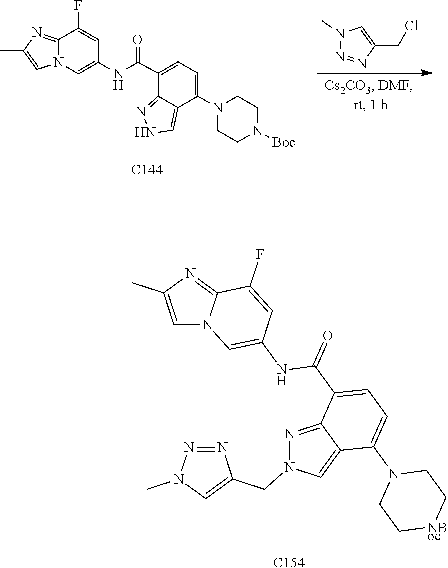

To a stirred mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.20 mmol, 1.0 equiv) and 4-(chloromethyl)-1-methyl-1,2,3-triazole (31.9 mg, 0.24 mmol, 1.2 equiv) in DMF (1 mL) was added Cs₂CO₃ (198.0 mg, 0.60 mmol, 3.0 equiv) at room temperature. The resulting mixture was stirred for 1 h at room temperature, then diluted with water and extracted with ethyl acetate (2×15 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with ethyl acetate to afford tert-butyl 4-[7-({8-fluoro-2-methylimidazo[1,2-a]pyridin-6-yl}carbamoyl)-2-[(1-methyl-1,2,3-triazol-4-yl)methyl]indazol-4-yl]piperazine-1-carboxylate (65 mg, 55%) as a solid. LCMS (ES, m/z): 589 [M+H]⁺.

### Synthesis of Compound 368

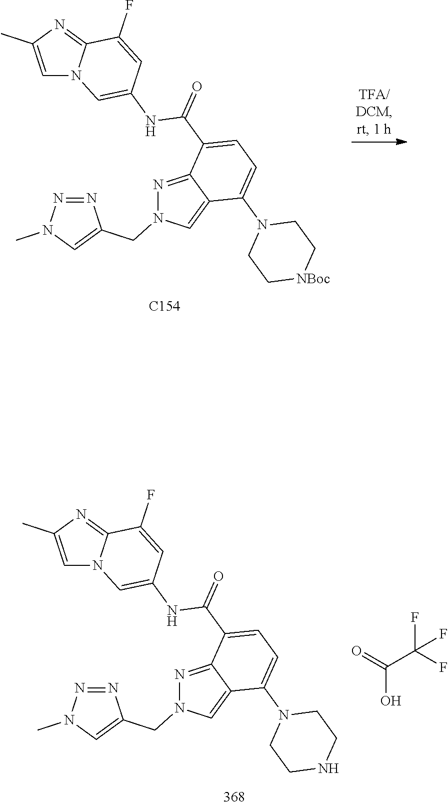 [SMILES: Cc1cn2cc(NC(=O)c3ccc(N4CCN(C(=O)OC(C)(C)C)CC4)c4cn(Cc5cn(C)nn5)nc34)cc(F)c2n1 ; "C154" ; Cc1cn2cc(NC(=O)c3ccc(N4CCNCC4)c4cn(Cc5cn(C)nn5)nc34)cc(F)c2n1 ; O=C(O)C(F)(F)F ; "368"]

A solution of tert-butyl 4-[7-({8-fluoro-2-methylimidazo[1,2-a]pyridin-6-yl}carbamoyl)-2-[(1-methyl-1,2,3-triazol-4-yl)methyl]indazol-4-yl]piperazine-1-carboxylate (65.0 mg, 0.11 mmol, 1.0 equiv) in DCM (0.3 mL) was treated with TFA (0.3 mL) at room temperature. The resulting mixture was stirred for 1 h at room temperature, then concentrated under reduced pressure to give a residue. The residue was purified by Prep-HPLC (Condition 3, Gradient 7) to afford N-{8-fluoro-2-methylimidazo[1,2-a]pyridin-6-yl}-2-[(1-methyl-1,2,3-triazol-4-yl)methyl]-4-(piperazin-1-yl) indazole-7-carboxamide; trifluoroacetic acid (25.8 mg, 39%) as a solid. LCMS (ES, m/z): 489 [M+H]⁺. ¹H NMR (400 MHz, DMSO-d₆) δ 11.22 (s, 1H), 9.40 (d, J=1.6 Hz, 1H), 9.04 (s, 1H), 8.90-8.89 (m, 2H), 8.26 (s, 1H), 8.08 (d, J=2.7 Hz, 1H), 8.03 (d, J=8.0 Hz, 1H), 7.55 (d, J=12.0 Hz, 1H), 6.63 (d, J=8.1 Hz, 1H), 5.89 (s, 2H), 4.06 (s, 3H), 3.60-3.59 (m, 4H), 3.36-3.34 (m, 4H), 2.42 (s, 3H).

### Example 119: Synthesis of Compound 369

### Synthesis of Intermediate C155

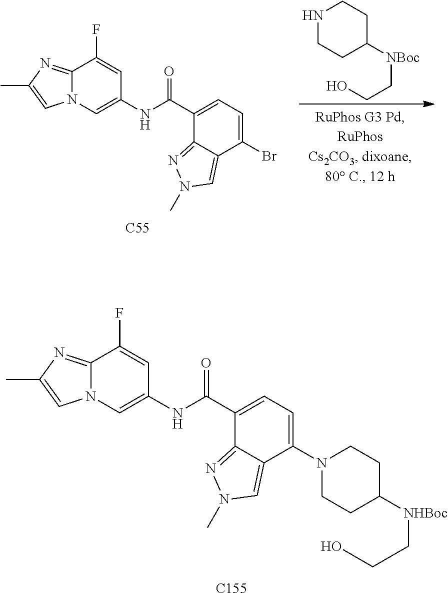

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-(2-hydroxyethyl)-N-(piperidin-4-yl)carbamate (43.74 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl N-{1-[7-({8-fluoro-2-methylimidazo[1,2-a]pyridin-6-yl}carbamoyl)-2-methylindazol-4-yl]piperidin-4-yl}-N-(2-hydroxyethyl)carbamate (70 mg, 83%) as an oil. LCMS (ES, m/z): 566[M+H]⁺.

### Synthesis of Compound 369

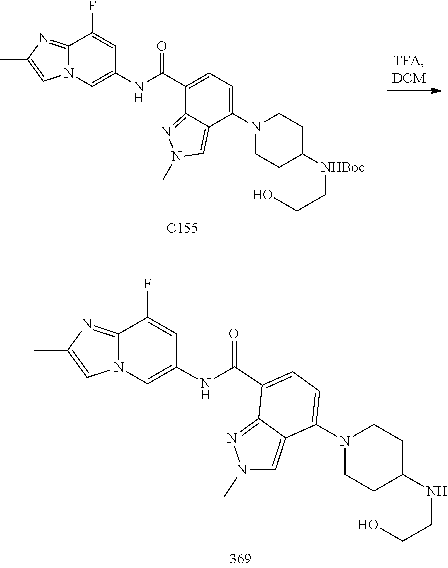

To a stirred solution of tert-butyl N-{1-[7-({8-fluoro-2-methylimidazo[1,2-a]pyridin-6-yl}carbamoyl)-2-methylindazol-4-yl]piperidin-4-yl}-N-(2-hydroxyethyl)carbamate (70 mg, 0.124 mmol, 1 equiv) in DCM (1 mL) was added TFA (1 mL, 13.463 mmol, 108.79 equiv) at room temperature. The resulting mixture was stirred for 1 h at room temperature, then concentrated under reduced pressure to give a residue. The residue was purified by Prep-HPLC (Condition 2, Gradient 8) to afford N-{8-fluoro-2-methylimidazo[1,2-a]pyridin-6-yl}-4-{4-[(2-hydroxyethyl)amino]piperidin-1-yl}-2-methylindazole-7-carboxamide (14.1 mg, 24%) as a solid. LCMS (ES, m/z): 466[M+H]⁺. ¹H NMR (300 MHz, DMSO-d₆) δ 11.07 (s, 1H), 9.21 (d, J=1.6 Hz, 1H), 8.78 (s, 1H), 7.97 (d, J=8.1 Hz, 1H), 7.91 (d, J=3.2 Hz, 1H), 7.34 (dd, J=12.4, 1.7 Hz, 1H), 6.56-6.45 (m, 2H), 4.57 (s, 1H), 4.30 (s, 3H), 3.91 (d, J=12.7 Hz, 2H), 3.50 (s, 2H), 3.06 (t, J=11.8 Hz, 2H), 2.70 (s, 3H), 2.35 (s, 3H), 1.99 (d, J=12.6 Hz, 2H), 1.47 (d, J=11.6 Hz, 2H).

### Example 120: Synthesis of Compound 370

### Synthesis of Intermediate C156

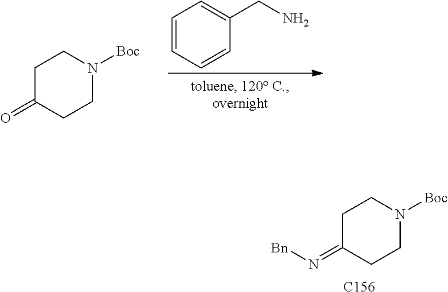 [SMILES: CC(C)(C)OC(=O)N1CCC(=O)CC1 ; NCc1ccccc1 ; CC(C)(C)OC(=O)N1CCC(=NCc2ccccc2)CC1 ; "C156"]

A mixture of tert-butyl 4-oxopiperidine-1-carboxylate (2.0 g, 10.038 mmol, 1 equiv) and benzylamine (1.08 g, 10.038 mmol, 1 equiv) in toluene (30 mL) was stirred overnight at 120° C. with an attached Dean-Stark trap. The resulting mixture was cooled to room temperature, then concentrated under vacuum to afford tert-butyl 4-(benzylimino)piperidine-1-carboxylate (3.05 g, crude) as an oil. LCMS (ES, m/z): 289[M+H]⁺.

### Synthesis of Intermediate C157

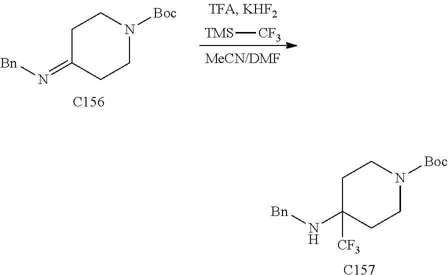 [SMILES: CC(C)(C)OC(=O)N1CCC(=NCc2ccccc2)CC1 ; C[Si](C)(C)C(F)(F)F ; "C156" ; CC(C)(C)OC(=O)N1CCC(NCc2ccccc2)(C(F)(F)F)CC1 ; "C157"]

To a stirred solution of tert-butyl 4-(benzylimino)piperidine-1-carboxylate (3.05 g, 10.576 mmol, 1 equiv), DMF (2.32 g, 31.728 mmol, 3 equiv), and KHF₂ (0.66 g, 8.461 mmol, 0.8 equiv) in acetonitrile (30 mL) was added TFA (1.51 g, 13.220 mmol, 1.25 equiv) dropwise at 0° C. under nitrogen atmosphere. The resulting mixture was stirred for 20 min at 0° C. under nitrogen atmosphere. To the reaction mixture was added trifluoromethyltrimethylsilane (2.26 g, 15.864 mmol, 1.5 equiv) dropwise at 0° C. under nitrogen atmosphere. The resulting mixture was stirred overnight at room temperature under nitrogen atmosphere, then quenched with saturated NaHCO₃ (50 mL) at room temperature. The resulting mixture was extracted with ethyl acetate (3×50 mL). The organic layers were combined, washed with brine (1×50 mL), dried over anhydrous Na₂SO₄, and filtered. After filtration, the filtrate was concentrated under reduced pressure to afford tert-butyl 4-(benzylamino)-4-(trifluoromethyl)piperidine-1-carboxylate (820 mg, 22%) as an oil. LCMS (ES, m/z): 359[M+H]⁺.

### Synthesis of Intermediate C158

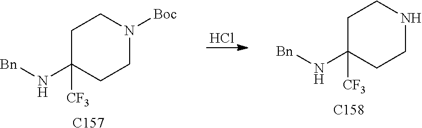 [SMILES: CC(C)(C)OC(=O)N1CCC(NCc2ccccc2)(C(F)(F)F)CC1 ; Cl ; FC(F)(F)C1(NCc2ccccc2)CCNCC1 ; "C158" ; "C157"]

A mixture of tert-butyl 4-(benzylamino)-4-(trifluoromethyl)piperidine-1-carboxylate (200 mg, 0.558 mmol, 1 equiv) and HCl (gas) in 1,4-dioxane (2 mL) was stirred for 2 h at room temperature under nitrogen atmosphere. The resulting mixture was concentrated under vacuum to afford N-benzyl-4-(trifluoromethyl)piperidin-4-amine dihydrochloride (180 mg, 97%) as a solid. LCMS (ES, m/z): 259[M+H]⁺.

### Synthesis of Intermediate C159

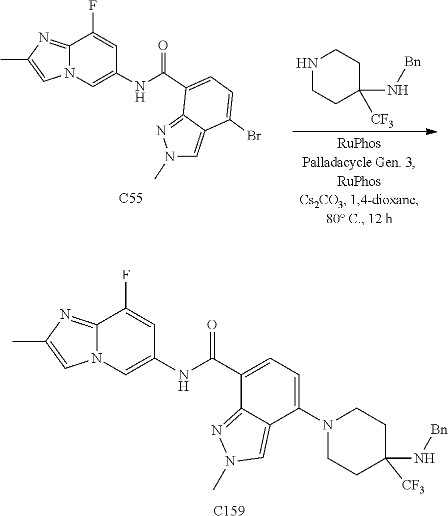 [SMILES: Cc1cn2cc(NC(=O)c3ccc(Br)c4cn(C)nc34)cc(F)c2n1 ; FC(F)(F)C1(NCc2ccccc2)CCNCC1 ; "C55" ; Cc1cn2cc(NC(=O)c3ccc(N4CCC(NCc5ccccc5)(C(F)(F)F)CC4)c4cn(C)nc34)cc(F)c2n1 ; "C159"]

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and N-benzyl-4-(trifluoromethyl)piperidin-4-amine (46.23 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford 4-[4-(benzylamino)-4-(trifluoromethyl)piperidin-1-yl]-N-{8-fluoro-2-methylimidazo[1,2-a]pyridin-6-yl}-2-methylindazole-7-carboxamide (70 mg, 81%) as an oil. LCMS (ES, m/z): 580[M+H]⁺.

### Synthesis of Compound 370

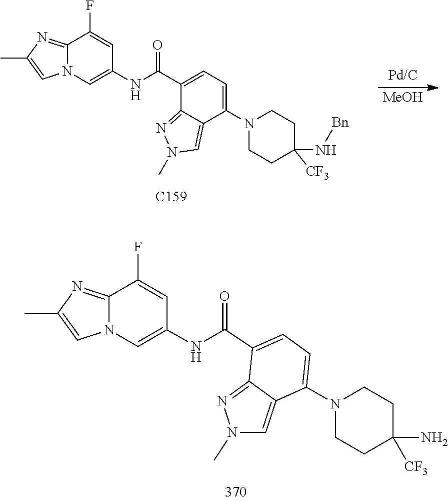 [SMILES: "MeOH" ; Cc1cn2cc(NC(=O)c3ccc(N4CCC(NCc5ccccc5)(C(F)(F)F)CC4)c4cn(C)nc34)cc(F)c2n1 ; "C159" ; Cc1cn2cc(NC(=O)c3ccc(N4CCC(N)(C(F)(F)F)CC4)c4cn(C)nc34)cc(F)c2n1]

To a solution of 4-[4-(benzylamino)-4-(trifluoromethyl)piperidin-1-yl]-N-{8-fluoro-2-methylimidazo[1,2-a]pyridin-6-yl}-2-methylindazole-7-carboxamide (70 mg, 0.121 mmol, 1 equiv) in methanol (5 mL) was added Pd/C (10%, 10 mg) under nitrogen atmosphere. The reaction mixture was hydrogenated at room temperature for 1 h using a hydrogen balloon. The resulting mixture was filtered, and the filtrate was concentrated under reduced pressure to give a residue. The residue was purified by Prep-HPLC (Condition 2, Gradient 2) to afford 4-[4-amino-4-(trifluoromethyl)piperidin-1-yl]-N-{8-fluoro-2-methylimidazo[1,2-a]pyridin-6-yl}-2-methylindazole-7-carboxamide (7.9 mg, 13%) as a solid. LCMS (ES, m/z): 490[M+H]⁺. ¹H NMR (400 MHz, Methanol-d₄) δ 9.09 (d, J=1.6 Hz, 1H), 8.52 (s, 1H), 8.09 (d, J=8.1 Hz, 1H), 7.75-7.70 (m, 1H), 7.26 (dd, J=11.8, 1.7 Hz, 1H), 6.57 (d, J=8.2 Hz, 1H), 4.85 (s, 3H), 4.33 (s, 2H), 3.85 (d, J=12.8 Hz, 2H), 3.49-3.39 (m, 2H), 2.44 (d, J=0.9 Hz, 3H), 2.17-2.03 (m, 2H), 1.76 (d, J=13.2 Hz, 2H), 1.31 (s, 1H).

### Example 121: Synthesis of Compound 371

### Synthesis of Intermediate C160

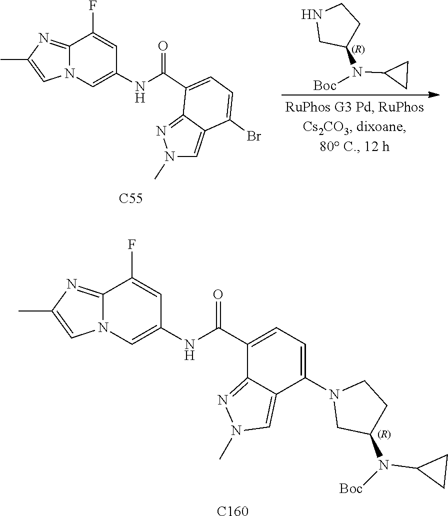

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-cyclopropyl-N-[(3R)-pyrrolidin-3-yl]carbamate (40.51 mg, 0.179 mmol, 1.2 equiv) in 1,4-dioxane (2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl N-cyclopropyl-N-[(3R)-1-[7-({8-fluoro-2-methylimidazo[1,2-a]pyridin-6-yl}carbamoyl)-2-methylindazol-4-yl]pyrrolidin-3-yl]carbamate (60 mg, 73%) as a solid. LCMS (ES, m/z): 548[M+H]⁺.

### Synthesis of Compound 371

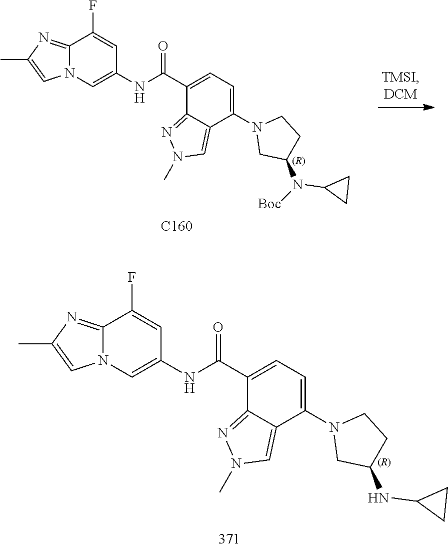

To a stirred solution of tert-butyl N-cyclopropyl-N-[(3R)-1-[7-({8-fluoro-2-methylimidazo[1,2-a]pyridin-6-yl}carbamoyl)-2-methylindazol-4-yl]pyrrolidin-3-yl]carbamate (60 mg, 0.110 mmol, 1 equiv) in DCM (1 mL) was added TMSI (109.61 mg, 0.550 mmol, 5 equiv) at room temperature. The resulting mixture was stirred for 2 h at room temperature, then neutralized with triethylamine. The resulting mixture was concentrated under reduced pressure to give a residue. The residue was purified by Prep-HPLC (Condition 2, Gradient 2), followed by chiral SFC (Condition 4, Gradient 1) to afford 4-[(3R)-3-(cyclopropylamino)pyrrolidin-1-yl]-N-{8-fluoro-2-methylimidazo[1,2-a]pyridin-6-yl}-2-methylindazole-7-carboxamide (17.8 mg, 36%) as a solid. LCMS (ES, m/z): 448[M+H]⁺. ¹H NMR (400 MHz, Methanol-d₄) δ 9.02 (d, J=1.8 Hz, 1H), 8.54 (s, 1H), 8.04 (d, J=8.4 Hz, 1H), 7.70 (d, J=3.0 Hz, 1H), 7.18 (dd, J=11.9, 1.6 Hz, 1H), 6.07 (d, J=8.5 Hz, 1H), 4.29 (s, 3H), 3.93-3.81 (m, 2H), 3.70 (dq, J=12.0, 6.8, 6.1 Hz, 2H), 3.53 (dd, J=10.3, 5.5 Hz, 1H), 2.44 (s, 3H), 2.40-2.26 (m, 2H), 2.15-2.02 (m, 1H), 0.58 (d, J=6.9 Hz, 2H), 0.45 (q, J=3.7 Hz, 2H).

### Example 122: Synthesis of Compound 372

### Synthesis of Intermediate C161

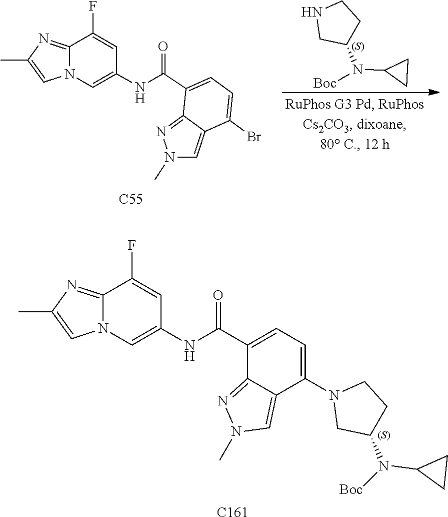

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-cyclopropyl-N—[(3S)-pyrrolidin-3-yl]carbamate (40.51 mg, 0.179 mmol, 1.2 equiv) in 1,4-dioxane (1.2 mL) was added Cs₂CO₃ (145.81 mg, 0.447 mmol, 3 equiv), RuPhos (13.92 mg, 0.030 mmol, 0.2 equiv), and RuPhos Palladacycle Gen.3 (12.48 mg, 0.015 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl N-cyclopropyl-N-[(3S)-1-[7-({8-fluoro-2-methylimidazo[1,2-a]pyridin-6-yl}carbamoyl)-2-methylindazol-4-yl]pyrrolidin-3-yl]carbamate (60 mg, 73%) as a solid. LCMS (ES, m/z): 548[M+H]⁺.

### Synthesis of Compound 372

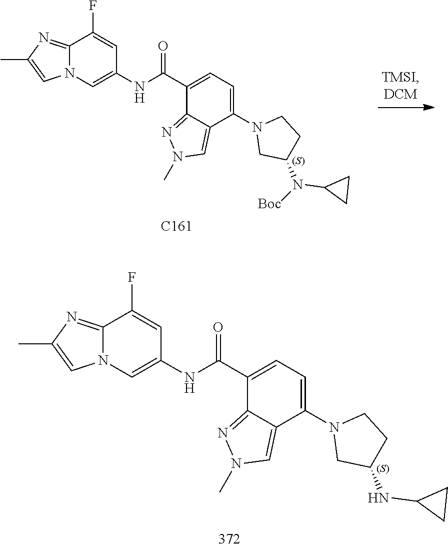

To a stirred solution of tert-butyl N-cyclopropyl-N-[(3R)-1-[7-({8-fluoro-2-methylimidazo[1,2-a]pyridin-6-yl}carbamoyl)-2-methylindazol-4-yl]pyrrolidin-3-yl]carbamate (60 mg, 0.110 mmol, 1 equiv) in DCM (1 mL) was added TMSI (109.61 mg, 0.550 mmol, 5 equiv) at room temperature. The resulting mixture was stirred for 2 h at room temperature, then neutralized with triethylamine. The resulting mixture was concentrated under reduced pressure to give a residue. The residue was purified by Prep-HPLC (Condition 12, Gradient 1). The product was further purified by chiral SFC (Condition 4, Gradient 1) to afford 4-[(3S)-3-(cyclopropylamino)pyrrolidin-1-yl]-N-{8-fluoro-2-methylimidazo[1,2-a]pyridin-6-yl}-2-methylindazole-7-carboxamide (9.3 mg, 19%) as a solid. LCMS (ES, m/z): 448[M+H]⁺. ¹H NMR (400 MHz, Methanol-d₄) δ 9.02 (d, J=1.6 Hz, 1H), 8.54 (s, 1H), 8.04 (d, J=8.4 Hz, 1H), 7.70 (d, J=3.0 Hz, 1H), 7.18 (dd, J=11.9, 1.7 Hz, 1H), 6.07 (d, J=8.4 Hz, 1H), 4.29 (s, 3H), 3.92-3.80 (m, 2H), 3.69 (dq, J=12.1, 6.7, 6.0 Hz, 2H), 3.57-3.50 (m, 1H), 2.44 (s, 3H), 2.40-2.26 (m, 2H), 2.12-2.04 (m, 2H), 0.61-0.52 (m, 2H), 0.44 (h, J=4.5, 3.9 Hz, 2H).

### Example 123: Synthesis of Compound 373

### Synthesis of Compound 373

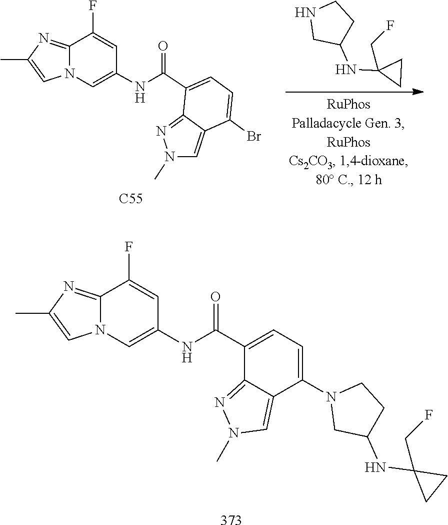 [SMILES: Cc1cn2cc(NC(=O)c3ccc(Br)c4cn(C)nc34)cc(F)c2n1 ; FCC1(NC2CCNC2)CC1 ; "C55" ; Cc1cn2cc(NC(=O)c3ccc(N4CCC(NC5(CF)CC5)C4)c4cn(C)nc34)cc(F)c2n1 ; "373"]

To a stirred mixture of 4-bromo-N-{8-fluoro-2-methylimidazo[1,2-a]pyridin-6-yl}-2-methylindazole-7-carboxamide (100 mg, 0.249 mmol, 1 equiv) and N-[1-(fluoromethyl)cyclopropyl]pyrrolidin-3-amine (47.20 mg, 0.299 mmol, 1.2 equiv) in 1,4-dioxane (2 mL) was added Cs₂CO₃ (243.01 mg, 0.747 mmol, 3 equiv), RuPhos (23.20 mg, 0.050 mmol, 0.2 equiv), and RuPhos Palladacycle Gen.3 (20.79 mg, 0.025 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1), followed by Prep-HPLC (Condition 12, Gradient 1) to afford N-{8-fluoro-2-methylimidazo[1,2-a]pyridin-6-yl}-4-(3-{[1-(fluoromethyl)cyclopropyl]amino}pyrrolidin-1-yl)-2-methylindazole-7-carboxamide (5.1 mg, 4%) as a solid. LCMS (ES, m/z): 480[M+H]⁺. ¹H NMR (400 MHz, Methanol-d4) δ 9.04 (d, J=1.7 Hz, 1H), 8.53 (s, 1H), 8.04 (d, J=8.4 Hz, 1H), 7.74-7.68 (m, 1H), 7.21 (dd, J=11.9, 1.6 Hz, 1H), 6.06 (d, J=8.4 Hz, 1H), 4.52 (s, 1H), 4.40 (s, 1H), 4.30 (s, 3H), 3.89 (q, J=7.8, 6.7 Hz, 3H), 3.85-3.78 (m, 1H), 3.71 (q, J=7.7 Hz, 1H), 2.44 (d, J=0.9 Hz, 3H), 2.34 (dd, J=12.1, 6.3 Hz, 1H), 2.04 (dt, J=13.9, 6.9 Hz, 1H), 1.31 (s, 2H), 0.84-0.67 (m, 4H).

### Example 124: Synthesis of Compound 374

### Synthesis of Intermediate C162

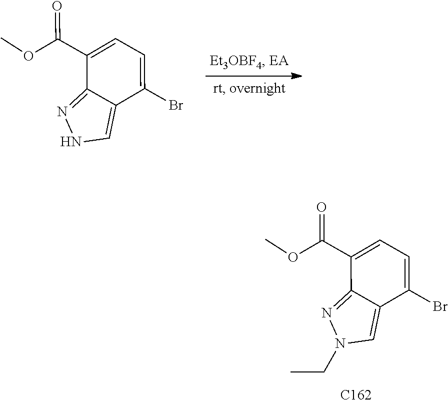

A mixture of methyl 4-bromo-2H-indazole-7-carboxylate (8 g, 31.36 mmol, 1 equiv) and tetrafluoroboranuide; triethyloxidanium (17.9 g, 94.09 mmol, 3 equiv) in ethyl acetate (100 mL) was stirred overnight at room temperature. The reaction mixture was quenched with water (200 mL) at room temperature, then extracted with ethyl acetate (3×50 mL). The organic layers were combined, washed with brine (1×100 mL), dried over anhydrous Na₂SO₄, and filtered. After filtration, the filtrate was concentrated under reduced pressure to afford methyl 4-bromo-2-ethylindazole-7-carboxylate (6.36 g, 72%) as a solid. LCMS (ES, m/z): 283 [M+H]⁺.

### Synthesis of Intermediate C163

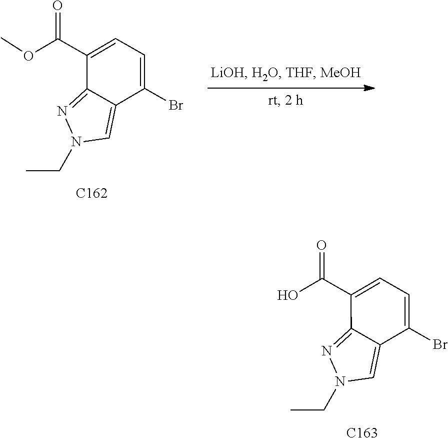

A mixture of methyl 4-bromo-2-ethylindazole-7-carboxylate (5.4 g, 18.93 mmol, 1 equiv) and LiOH·H₂O (7.9 g, 189.3 mmol, 10 equiv) in THF (50 mL), methanol (50 mL) and water (25 mL) was stirred for 2 h at room temperature. The resulting mixture was concentrated under reduced pressure to give a residue. The residue was dissolved in ethyl acetate (100 mL) and diluted with water (200 mL). The resulting mixture was acidified to pH 2 with 1M HCl (aq.) and extracted with ethyl acetate (3×50 mL). The organic layers were combined, washed with brine (1×100 mL), dried over anhydrous Na₂SO₄, and filtered. After filtration, the filtrate was concentrated under reduced pressure to afford 4-bromo-2-ethylindazole-7-carboxylic acid (5 g, 98%) as a solid. LCMS (ES, m/z): 269 [M+H]⁺.

### Synthesis of Intermediate C164

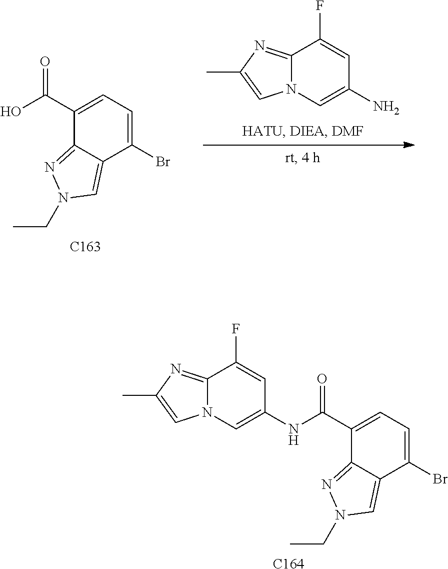 [SMILES: CCn1cc2c(Br)ccc(C(=O)O)c2n1 ; Cc1cn2cc(N)cc(F)c2n1 ; "C163" ; CCn1cc2c(Br)ccc(C(=O)Nc3cc(F)c4nc(C)cn4c3)c2n1 ; "C164"]

To a stirred mixture of 4-bromo-2-ethylindazole-7-carboxylic acid (5.8 g, 21.55 mmol, 1 equiv) and 8-fluoro-2-methylimidazo[1,2-a]pyridin-6-amine (5.3 g, 32.33 mmol, 1.5 equiv) in DMF (20 mL) was added HATU (9.8 g, 25.86 mmol, 1.2 equiv) and DIEA (13.9 g, 107.76 mmol, 5 equiv) in portions at room temperature. The resulting mixture was stirred for 4 h at room temperature, then quenched with water (50 mL). A precipitate formed that was collected by filtration and washed with water (2×50 mL) to afford 4-bromo-2-ethyl-N-{8-fluoro-2-methylimidazo[1,2-a]pyridin-6-yl}indazole-7-carboxamide (8.1 g, 90%) as a solid. LCMS (ES, m/z): 416 [M+H]⁺.

### Synthesis of Intermediate C165

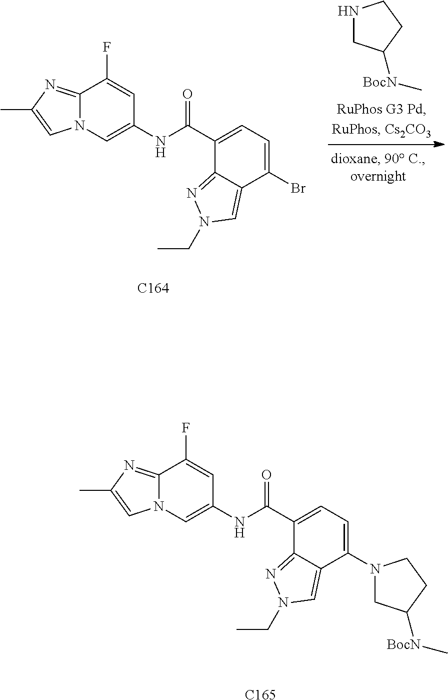

To a stirred mixture of 4-bromo-2-ethyl-N-{8-fluoro-2-methylimidazo[1,2-a]pyridin-6-yl}indazole-7-carboxamide (180 mg, 0.43 mmol, 1 equiv) and tert-butyl N-methyl-N-(pyrrolidin-3-yl)carbamate (87 mg, 0.43 mmol, 1 equiv) in dioxane (10 mL) was added Cs₂CO₃ (423 mg, 1.29 mmol, 3 equiv), RuPhos (41 mg, 0.086 mmol, 0.2 equiv), and RuPhos Palladacycle Gen.3 (36 mg, 0.043 mmol, 0.1 equiv) in portions at room temperature under nitrogen atmosphere. The resulting mixture was stirred overnight at 90° C. under nitrogen atmosphere, then concentrated under reduced pressure to give a residue. The residue was taken up in water (20 mL) and extracted with ethyl acetate (3×10 mL). The organic layers were combined, washed with brine (1×30 mL), dried over anhydrous Na₂SO₄, and filtered. After filtration, the filtrate was concentrated under reduced pressure to give a residue. The residue was purified by reverse flash chromatography (Condition 2, Gradient 2) to afford tert-butyl N-{1-[2-ethyl-7-({8-fluoro-2-methylimidazo[1,2-a]pyridin-6-yl}carbamoyl) indazol-4-yl]pyrrolidin-3-yl}-N-methylcarbamate (160 mg, 69%) as a solid. LCMS (ES, m/z): 536 [M+H]⁺.

### Synthesis of Compound 374

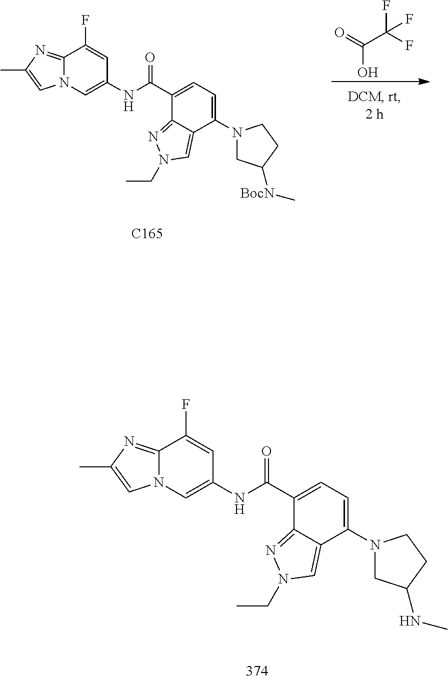

A mixture of tert-butyl N-{1-[2-ethyl-7-({8-fluoro-2-methylimidazo[1,2-a]pyridin-6-yl}carbamoyl) indazol-4-yl]pyrrolidin-3-yl}-N-methylcarbamate (135 mg, 0.25 mmol, 1 equiv) in trifluoroacetic acid (2 mL) and DCM (2 mL) was stirred for 2 h at room temperature. The resulting mixture was concentrated under reduced pressure to give a residue. The residue was basified to pH 8 with 7 M NH₃(g) in methanol. The resulting mixture was concentrated under reduced pressure to give a residue. The residue was purified by prep-HPLC (Condition 13, Gradient 1) to afford 2-ethyl-N-{8-fluoro-2-methylimidazo[1,2-a]pyridin-6-yl}-4-[3-(methylamino)pyrrolidin-1-yl]indazole-7-carboxamide (30 mg, 27%) as a solid. LCMS (ES, m/z): 436 [M+H]⁺. ¹H NMR (300 MHz, DMSO-d6) δ 11.05 (s, 1H), 9.19 (d, J=1.6 Hz, 1H), 8.84 (s, 1H), 7.97-7.85 (m, 2H), 7.26 (dd, J=12.4, 1.6 Hz, 1H), 6.01 (d, J=8.4 Hz, 1H), 4.57 (q, J=7.3 Hz, 2H), 3.76 (dq, J=13.7, 7.1, 6.3 Hz, 1H), 3.65 (d, J=7.4 Hz, 3H), 3.42 (dd, J=10.2, 4.0 Hz, 2H), 2.35 (s, 6H), 2.14 (dd, J=11.2, 4.5 Hz, 1H), 1.92 (dd, J=11.8, 6.1 Hz, 1H), 1.61 (t, J=7.2 Hz, 3H).

### Example 125: Synthesis of Compound 375

### Synthesis of Compound 375

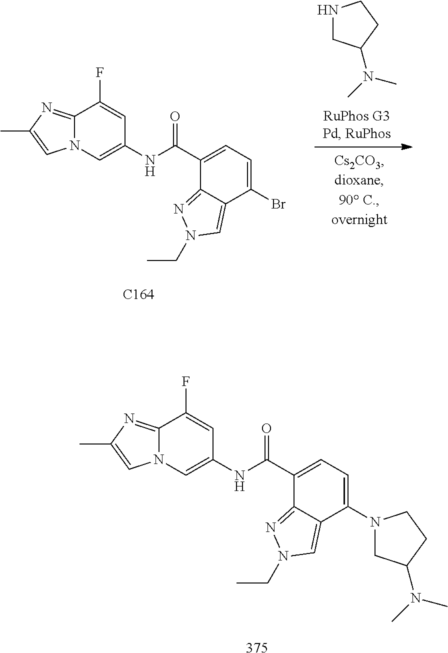

To a stirred mixture of 4-bromo-2-ethyl-N-{8-fluoro-2-methylimidazo[1,2-a]pyridin-6-yl}indazole-7-carboxamide (90 mg, 0.22 mmol, 1 equiv) and N,N-dimethylpyrrolidin-3-amine (25 mg, 0.22 mmol, 1 equiv) in dioxane (5 mL) was added Cs₂CO₃ (211 mg, 0.66 mmol, 3 equiv), RuPhos (20 mg, 0.044 mmol, 0.2 equiv), and RuPhos Palladacycle Gen.3 (18 mg, 0.022 mmol, 0.1 equiv) in portions at room temperature under nitrogen atmosphere. The resulting mixture was stirred overnight at 90° C. under nitrogen atmosphere, then cooled to room temperature. The resulting mixture was concentrated under reduced pressure to give a residue. The residue was dissolved in water (20 mL) and extracted with ethyl acetate (3×5 mL). The organic layers were combined, washed with brine (1×10 mL), dried over anhydrous Na₂SO₄, and filtered. After filtration, the filtrate was concentrated under reduced pressure to give a residue. The residue was purified by prep-HPLC (Condition 13, Gradient 2) to afford 4-[3-(dimethylamino)pyrrolidin-1-yl]-2-ethyl-N-{8-fluoro-2-methylimidazo[1,2-a]pyridin-6-yl}indazole-7-carboxamide (17.4 mg, 18%) as a solid. LCMS (ES, m/z): 450 [M+H]⁺. ¹H NMR (300 MHz, DMSO-d₆) δ 11.06 (s, 1H), 9.19 (d, J=1.6 Hz, 1H), 8.88 (s, 1H), 7.98-7.86 (m, 2H), 7.27 (dd, J=12.4, 1.7 Hz, 1H), 6.05 (d, J=8.4 Hz, 1H), 4.57 (q, J=7.3 Hz, 2H), 3.81 (dt, J=19.0, 9.0 Hz, 2H), 3.65 (q, J=9.5, 9.0 Hz, 1H), 3.46 (t, J=9.0 Hz, 1H), 2.91 (s, 1H), 2.35 (s, 3H), 2.28 (s, 6H), 2.23 (s, 1H), 1.93 (q, J=11.6, 10.7 Hz, 1H), 1.61 (t, J=7.2 Hz, 3H).

### Example 126: Synthesis of Compound 376

### Synthesis of Intermediate C166

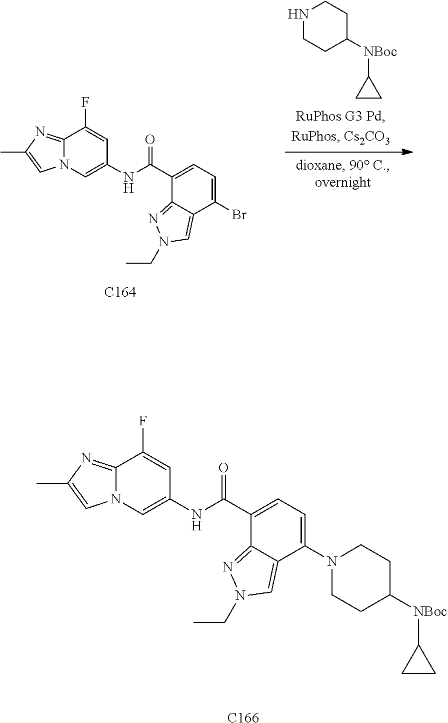 [SMILES: CC(C)(C)OC(=O)N(C1CCNCC1)C1CC1 ; CCn1cc2c(Br)ccc(C(=O)Nc3cc(F)c4nc(C)cn4c3)c2n1 ; "C164" ; CCn1cc2c(N3CCC(N(C(=O)OC(C)(C)C)C4CC4)CC3)ccc(C(=O)Nc3cc(F)c4nc(C)cn4c3)c2n1 ; "C166"]

To a stirred solution of 4-bromo-2-ethyl-N-{8-fluoro-2-methylimidazo[1,2-a]pyridin-6-yl}indazole-7-carboxamide (190 mg, 0.46 mmol, 1 equiv) and tert-butyl N-cyclopropyl-N-(piperidin-4-yl)carbamate (110 mg, 0.46 mmol, 1 equiv) in dioxane (10 mL) was added Cs₂CO₃ (446 mg, 1.38 mmol, 3 equiv), RuPhos (43 mg, 0.092 mmol, 0.2 equiv), and RuPhos Palladacycle Gen.3 (38 mg, 0.046 mmol, 0.1 equiv) in portions at room temperature under nitrogen atmosphere. The resulting mixture was stirred overnight at 90° C. under nitrogen atmosphere, then cooled to room temperature. The resulting mixture was concentrated under reduced pressure to give a residue. The residue was dissolved in water (20 mL) and extracted with ethyl acetate (3×5 mL). The organic layers were combined, washed with brine (1×10 mL), dried over anhydrous Na₂SO₄, and filtered. After filtration, the filtrate was concentrated under reduced pressure to give a residue. The residue was purified by reverse flash chromatography (Condition 2, Gradient 2) to afford tert-butyl N-cyclopropyl-N-{1-[2-ethyl-7-({8-fluoro-2-methylimidazo[1,2-a]pyridin-6-yl}carbamoyl) indazol-4-yl]piperidin-4-yl}carbamate (150 mg, 57%) as a solid. LCMS (ES, m/z): 576 [M+H]⁺.

### Synthesis of Compound 376

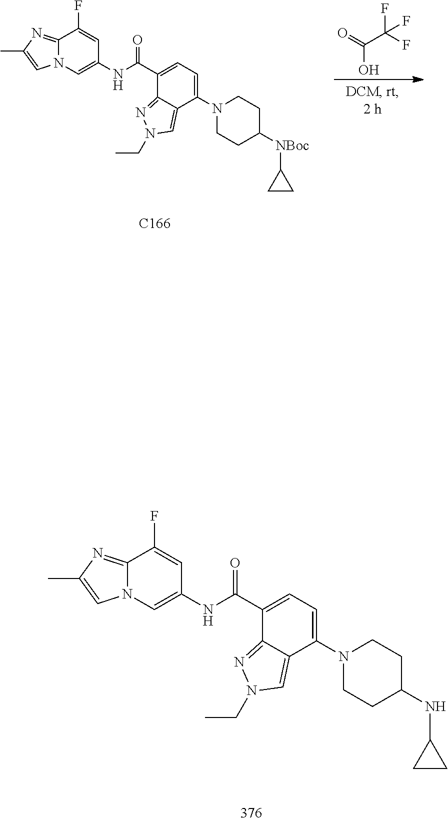 [SMILES: CCn1cc2c(N3CCC(N(C(=O)OC(C)(C)C)C4CC4)CC3)ccc(C(=O)Nc3cc(F)c4nc(C)cn4c3)c2n1 ; O=C(O)C(F)(F)F ; "C166" ; CCn1cc2c(N3CCC(NC4CC4)CC3)ccc(C(=O)Nc3cc(F)c4nc(C)cn4c3)c2n1 ; "376"]

A mixture of tert-butyl N-cyclopropyl-N-{1-[2-ethyl-7-({8-fluoro-2-methylimidazo[1,2-a]pyridin-6-yl}carbamoyl) indazol-4-yl]piperidin-4-yl}carbamate (60 mg, 0.104 mmol, 1 equiv) in trifluoroacetic acid (2 mL) and DCM (2 mL) was stirred for 2 h at room temperature. The resulting mixture was concentrated under reduced pressure to give a residue. The residue was basified to pH 8 with 7 M NH₃(g) in methanol, then concentrated under vacuum to give a residue. The residue was purified by prep-HPLC (Condition 13, Gradient 3) to afford 4-[4-(cyclopropylamino)piperidin-1-yl]-2-ethyl-N-{8-fluoro-2-methylimidazo[1,2-a]pyridin-6-yl}indazole-7-carboxamide (20 mg, 40%) as a solid. LCMS (ES, m/z): 476 [M+H]⁺. ¹H NMR (300 MHz, DMSO-d₆) δ 11.10 (s, 1H), 9.21 (d, J=1.6 Hz, 1H), 8.80 (s, 1H), 7.97 (d, J=8.1 Hz, 1H), 7.90 (d, J=3.1 Hz, 1H), 7.30 (dd, J=12.3, 1.7 Hz, 1H), 6.48 (d, J=8.2 Hz, 1H), 4.60 (q, J=7.2 Hz, 2H), 3.88 (d, J=13.0 Hz, 2H), 3.13-2.99 (m, 2H), 2.77 (dd, J=9.2, 5.0 Hz, 1H), 2.35 (s, 3H), 2.13 (tt, J=6.8, 3.6 Hz, 1H), 2.06-1.96 (m, 2H), 1.62 (t, J=7.3 Hz, 3H), 1.48 (q, J=10.2 Hz, 2H), 0.40 (dt, J=6.2, 3.0 Hz, 2H), 0.24 (p, J=3.9 Hz, 2H).

### Example 127: Synthesis of Compound 377

### Synthesis of Intermediate C167

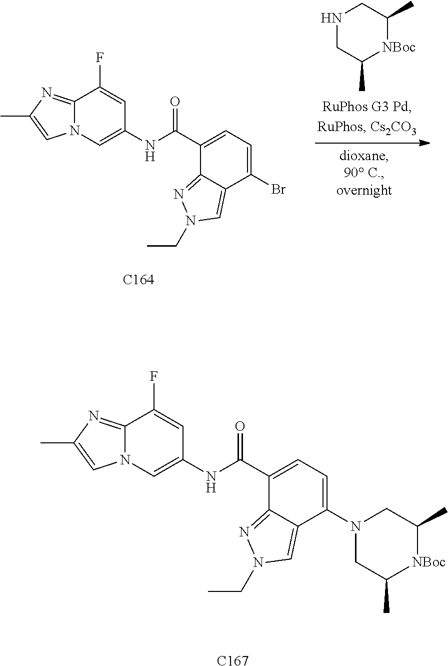

To a stirred mixture of 4-bromo-2-ethyl-N-{8-fluoro-2-methylimidazo[1,2-a]pyridin-6-yl}indazole-7-carboxamide (90 mg, 0.22 mmol, 1 equiv) and tert-butyl (2R,6S)-2,6-dimethylpiperazine-1-carboxylate (50 mg, 0.24 mmol, 1.0 equiv) in dioxane (5 mL) was added Cs₂CO₃ (211 mg, 0.648 mmol, 3 equiv), RuPhos (20 mg, 0.044 mmol, 0.2 equiv), and RuPhos Palladacycle Gen.3 (18 mg, 0.022 mmol, 0.1 equiv) in portions at room temperature under nitrogen atmosphere. The resulting mixture was stirred overnight at 90° C. under nitrogen atmosphere, then cooled to room temperature. The resulting mixture was concentrated under reduced pressure to give a residue. The residue was dissolved in water (20 mL) and extracted with ethyl acetate (3×5 mL). The organic layers were combined, washed with brine (1×10 mL), dried over anhydrous Na₂SO₄, and filtered. After filtration, the filtrate was concentrated under reduced pressure to give a residue. The residue was purified by reverse flash chromatography (Condition 2, Gradient 2) to afford tert-butyl (2R,6S)-4-[2-ethyl-7-({8-fluoro-2-methylimidazo[1,2-a]pyridin-6-yl}carbamoyl) indazol-4-yl]-2,6-dimethylpiperazine-1-carboxylate (65 mg, 55%) as a solid. LCMS (ES, m/z): 550 [M+H]⁺.

### Synthesis of Compound 377

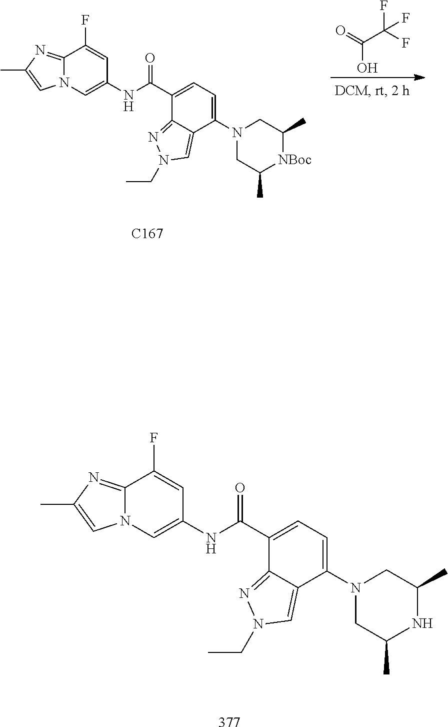

A mixture of tert-butyl (2R,6S)-4-[2-ethyl-7-({8-fluoro-2-methylimidazo[1,2-a]pyridin-6-yl}carbamoyl) indazol-4-yl]-2,6-dimethylpiperazine-1-carboxylate (10 mg, 0.018 mmol, 1 equiv) in trifluoroacetic acid (2 mL) and DCM (2 mL) was stirred for 2 h at room temperature. The resulting mixture was concentrated under reduced pressure, then basified to pH 8 with 7 M NH₃ (g) in methanol, and concentrated under vacuum to give a residue. The residue was purified by prep-HPLC (Condition 13, Gradient 4) to afford 4-[(3R,5S)-3,5-dimethylpiperazin-1-yl]-2-ethyl-N-{8-fluoro-2-methylimidazo[1,2-a]pyridin-6-yl}indazole-7-carboxamide (12 mg, 27%) as a solid. LCMS (ES, m/z): 450 [M+H]⁺. ¹H NMR (300 MHz, DMSO-d₆) δ 11.11 (s, 1H), 9.20 (d, J=1.6 Hz, 1H), 8.82 (s, 1H), 7.97 (d, J=8.1 Hz, 1H), 7.90 (d, J=3.1 Hz, 1H), 7.29 (dd, J=12.3, 1.7 Hz, 1H), 6.47 (d, J=8.2 Hz, 1H), 4.60 (q, J=7.3 Hz, 2H), 3.78 (dd, J=12.2, 2.7 Hz, 2H), 2.96 (tt, J=8.0, 5.5 Hz, 2H), 2.44 (d, J=11.0 Hz, 2H), 2.35 (s, 3H), 1.62 (t, J=7.2 Hz, 3H), 1.07 (d, J=6.2 Hz, 6H).

### Example 128: Synthesis of Compound 378

### Synthesis of Compound 378

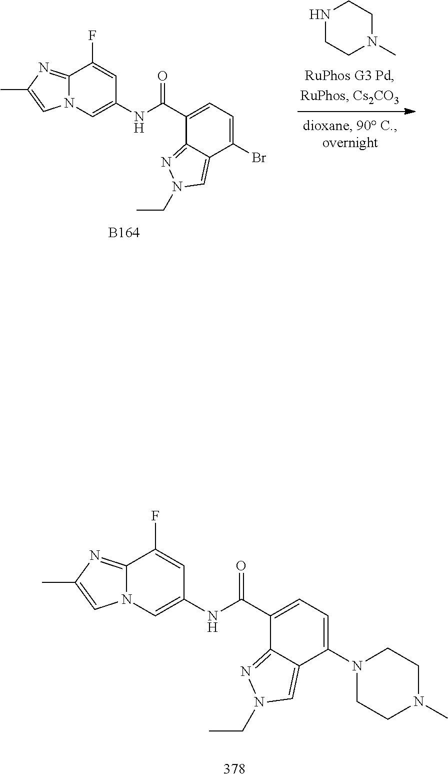

To a stirred mixture of 4-bromo-2-ethyl-N-{8-fluoro-2-methylimidazo[1,2-a]pyridin-6-yl}indazole-7-carboxamide (90 mg, 0.22 mmol, 1 equiv) and 1-methylpiperazine (22 mg, 0.22 mmol, 1 equiv) in dioxane (5 mL) was added Cs₂CO₃ (211 mg, 0.66 mmol, 3 equiv), RuPhos (20 mg, 0.044 mmol, 0.2 equiv), and RuPhos Palladacycle Gen.3 (18 mg, 0.022 mmol, 0.1 equiv) in portions at room temperature under nitrogen atmosphere. The resulting mixture was stirred overnight at 90° C. under nitrogen atmosphere, then cooled to room temperature, and concentrated under reduced pressure to give a residue. The residue was dissolved in water (20 mL) and extracted with ethyl acetate (3×5 mL). The organic layers were combined, washed with brine (1×10 mL), dried over anhydrous Na₂SO₄, and filtered. After filtration, the filtrate was concentrated under reduced pressure to give a residue. The residue was purified by prep-HPLC (Condition 13, Gradient 2) to afford 2-ethyl-N-{8-fluoro-2-methylimidazo[1,2-a]pyridin-6-yl}-4-(4-methylpiperazin-1-yl) indazole-7-carboxamide (15 mg, 16%) as a solid. LCMS (ES, m/z): 436 [M+H]⁺. ¹H NMR (300 MHz, DMSO-d₆) δ 11.11 (s, 1H), 9.21 (d, J=1.6 Hz, 1H), 8.84 (s, 1H), 7.98 (d, J=8.1 Hz, 1H), 7.90 (dd, J=3.1, 1.0 Hz, 1H), 7.30 (dd, J=12.4, 1.7 Hz, 1H), 6.51 (d, J=8.2 Hz, 1H), 4.60 (q, J=7.3 Hz, 2H), 3.43 (t, J=5.0 Hz, 4H), 2.55 (d, J=4.9 Hz, 4H), 2.35 (s, 3H), 2.28 (s, 3H), 1.62 (t, J=7.3 Hz, 3H).

### Example 129: Synthesis of Compound 379

### Synthesis of Intermediate C168

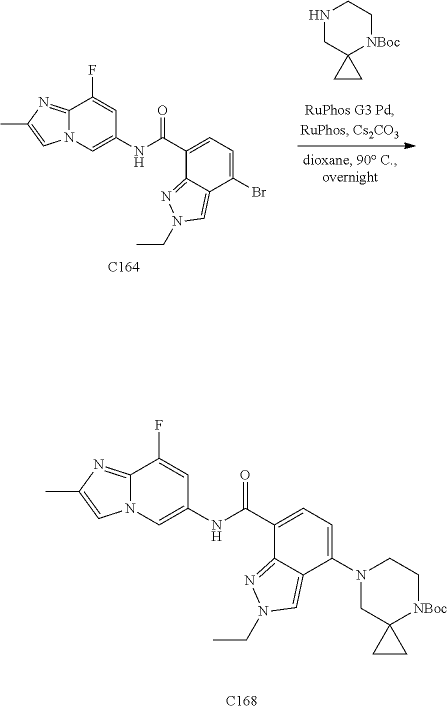 [SMILES: CCn1cc2c(Br)ccc(C(=O)Nc3cc(F)c4nc(C)cn4c3)c2n1 ; CC(C)(C)OC(=O)N1CCNCC12CC2 ; "C164" ; CCn1cc2c(N3CCN(C(=O)OC(C)(C)C)C4(CC4)C3)ccc(C(=O)Nc3cc(F)c4nc(C)cn4c3)c2n1 ; "C168"]

To a stirred mixture of 4-bromo-2-ethyl-N-{8-fluoro-2-methylimidazo[1,2-a]pyridin-6-yl}indazole-7-carboxamide (90 mg, 0.22 mmol, 1 equiv) and tert-butyl 4,7-diazaspiro[2.5]octane-4-carboxylate (46 mg, 0.22 mmol, 1 equiv) in dioxane (5 mL) was added Cs₂CO₃ (211 mg, 0.66 mmol, 3 equiv), RuPhos (10 mg, 0.044 mmol, 0.2 equiv), and RuPhos Palladacycle Gen.3 (18 mg, 0.022 mmol, 0.1 equiv) in portions at room temperature under nitrogen atmosphere. The resulting mixture was stirred overnight at 90° C. under nitrogen atmosphere, then cooled to room temperature, and concentrated under vacuum to give a residue. The residue was dissolved in water (20 mL) and extracted with ethyl acetate (3×5 mL). The organic layers were combined, washed with brine (1×10 mL), dried over anhydrous Na₂SO₄, and filtered. After filtration, the filtrate was concentrated under reduced pressure to give a residue. The residue was purified by reverse flash chromatography (Condition 2, Gradient 2) to afford tert-butyl 7-[2-ethyl-7-({8-fluoro-2-methylimidazo[1,2-a]pyridin-6-yl}carbamoyl) indazol-4-yl]-4,7-diazaspiro[2.5]octane-4-carboxylate (50 mg, 42%) as a solid. LCMS (ES, m/z): 548 [M+H]⁺.

### Synthesis of Compound 379

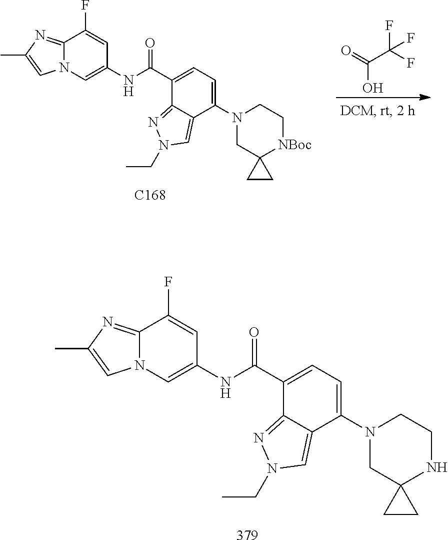 [SMILES: CCn1cc2c(N3CCN(C(=O)OC(C)(C)C)C4(CC4)C3)ccc(C(=O)Nc3cc(F)c4nc(C)cn4c3)c2n1 ; O=C(O)C(F)(F)F ; "C168" ; CCn1cc2c(N3CCNC4(CC4)C3)ccc(C(=O)Nc3cc(F)c4nc(C)cn4c3)c2n1 ; "379"]

A mixture of tert-butyl 7-[2-ethyl-7-({8-fluoro-2-methylimidazo[1,2-a]pyridin-6-yl}carbamoyl) indazol-4-yl]-4,7-diazaspiro[2.5]octane-4-carboxylate (40 mg, 0.073 mmol, 1 equiv) in trifluoroacetic acid (2 mL) and DCM (2 mL) was stirred for 2 h at room temperature. The resulting mixture was concentrated under vacuum, basified to pH 8 with 7 M NH₃(g) in methanol, and concentrated under vacuum to give a residue. The residue was purified by prep-HPLC (Condition 13, Gradient 3) to afford 4-{4,7-diazaspiro[2.5]octan-7-yl}-2-ethyl-N-{8-fluoro-2-methylimidazo[1,2-a]pyridin-6-yl}indazole-7-carboxamide (20 mg, 61%) as a solid. LCMS (ES, m/z): 448 [M+H]⁺. ¹H NMR (300 MHz, DMSO-d₆) δ 11.11 (s, 1H), 9.20 (s, 1H), 8.73 (s, 1H), 7.97 (d, J=8.1 Hz, 1H), 7.93-7.87 (m, 1H), 7.30 (d, J=12.3 Hz, 1H), 6.46 (d, J=8.2 Hz, 1H), 4.59 (q, J=7.2 Hz, 2H), 3.40 (s, 2H), 3.25 (s, 2H), 2.99 (d, J=6.0 Hz, 2H), 2.35 (s, 3H), 1.62 (t, J=7.3 Hz, 3H), 0.61 (s, 2H), 0.54 (s, 2H).

### Example 130: Synthesis of Compound 380

### Synthesis of Intermediate C169

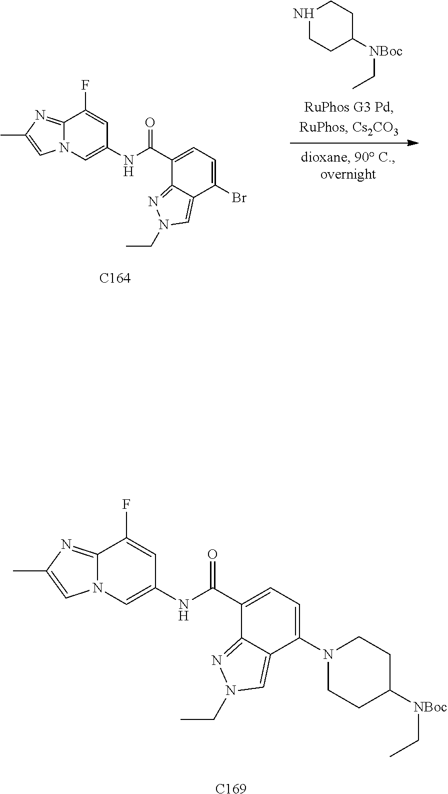 [SMILES: CCn1cc2c(Br)ccc(C(=O)Nc3cc(F)c4nc(C)cn4c3)c2n1 ; CCN(C(=O)OC(C)(C)C)C1CCNCC1 ; "C164" ; CCN(C(=O)OC(C)(C)C)C1CCN(c2ccc(C(=O)Nc3cc(F)c4nc(C)cn4c3)c3nn(CC)cc23)CC1 ; "C169"]

To a stirred solution of 4-bromo-2-ethyl-N-{8-fluoro-2-methylimidazo[1,2-a]pyridin-6-yl}indazole-7-carboxamide (90 mg, 0.216 mmol, 1 equiv) and tert-butyl N-ethyl-N-(piperidin-4-yl)carbamate (49 mg, 0.216 mmol, 1 equiv) in dioxane (5 mL) was added Cs₂CO₃ (211 mg, 0.648 mmol, 3 equiv), RuPhos (11 mg, 0.044 mmol, 0.2 equiv), and RuPhos Palladacycle Gen.3 (10 mg, 0.022 mmol, 0.1 equiv) in portions at room temperature under nitrogen atmosphere. The resulting mixture was stirred overnight at 90° C. under nitrogen atmosphere, then cooled to room temperature, and concentrated under reduced pressure to give a residue. The residue was dissolved in water (20 mL) and extracted with ethyl acetate (3×5 mL). The organic layers were combined, washed with brine (1×10 mL), dried over anhydrous Na₂SO₄, and filtered. After filtration, the filtrate was concentrated under reduced pressure to give a residue. The residue was purified by reverse flash chromatography (Condition 2, Gradient 2) to afford tert-butyl N-ethyl-N-{1-[2-ethyl-7-({8-fluoro-2-methylimidazo[1,2-a]pyridin-6-yl}carbamoyl) indazol-4-yl]piperidin-4-yl}carbamate (45 mg, 37%) as a solid. LCMS (ES, m/z): 564 [M+H]⁺.

### Synthesis of Compound 380

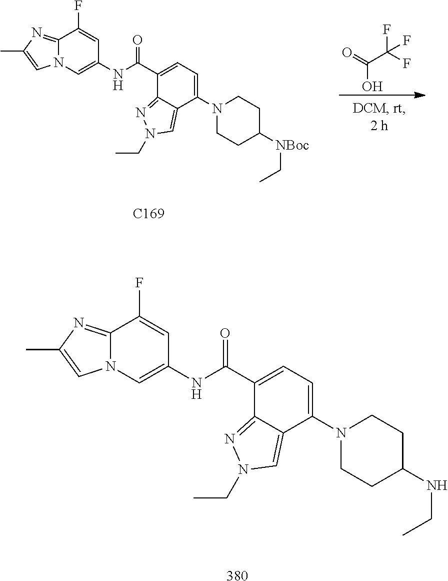 [SMILES: CCN(C(=O)OC(C)(C)C)C1CCN(c2ccc(C(=O)Nc3cc(F)c4nc(C)cn4c3)c3nn(CC)cc23)CC1 ; O=C(O)C(F)(F)F ; "C169" ; CCNC1CCN(c2ccc(C(=O)Nc3cc(F)c4nc(C)cn4c3)c3nn(CC)cc23)CC1 ; "380"]

A mixture of tert-butyl N-ethyl-N-{1-[2-ethyl-7-({8-fluoro-2-methylimidazo[1,2-a]pyridin-6-yl}carbamoyl) indazol-4-yl]piperidin-4-yl}carbamate (35 mg, 0.062 mmol, 1 equiv) in trifluoroacetic acid (1 mL) and DCM (1 mL) was stirred for 2 h at room temperature. The resulting mixture was concentrated under reduced pressure, basified to pH 8 with 7 M NH₃(g) in methanol, and concentrated under vacuum to give a residue. The residue was purified by prep-HPLC (Condition 13, Gradient 3) to afford 2-ethyl-4-[4-(ethylamino)piperidin-1-yl]-N-{8-fluoro-2-methylimidazo[1,2-a]pyridin-6-yl}indazole-7-carboxamide (15 mg, 52%) as a solid. LCMS (ES, m/z): 464 [M+H]⁺. ¹H NMR (300 MHz, DMSO-d₆) δ 11.10 (s, 1H), 9.21 (d, J=1.6 Hz, 1H), 8.79 (s, 1H), 7.97 (d, J=8.1 Hz, 1H), 7.90 (d, J=3.1 Hz, 1H), 7.30 (dd, J=12.3, 1.7 Hz, 1H), 6.48 (d, J=8.2 Hz, 1H), 4.60 (q, J=7.2 Hz, 2H), 3.89 (d, J=12.7 Hz, 2H), 3.06 (t, J=11.4 Hz, 2H), 2.75-2.55 (m, 3H), 2.35 (s, 3H), 1.96 (d, J=12.1 Hz, 2H), 1.62 (t, J=7.2 Hz, 3H), 1.45 (q, J=10.0, 9.6 Hz, 2H), 1.04 (t, J=7.1 Hz, 3H).

### Example 131: Synthesis of Compound 381

### Synthesis of Compound 381

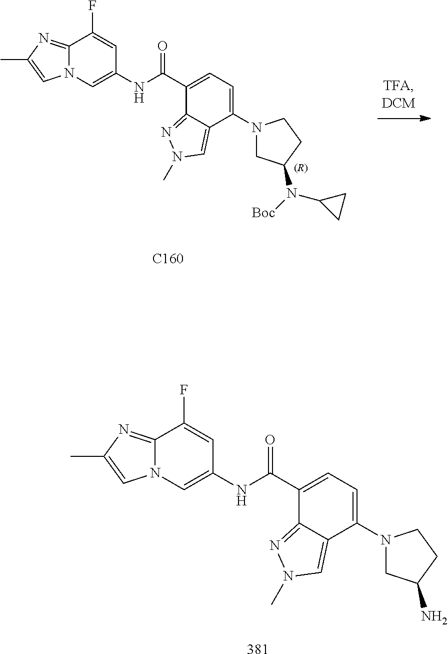

To a stirred solution of tert-butyl N-cyclopropyl-N-[(3R)-1-[7-({8-fluoro-2-methylimidazo[1,2-a]pyridin-6-yl}carbamoyl)-2-methylindazol-4-yl]pyrrolidin-3-yl]carbamate (60 mg, 0.110 mmol, 1 equiv) in DCM (1 mL, 143.58 equiv) was added TFA (1 mL, 122.88 equiv) at room temperature. The resulting mixture was stirred for 2 h at room temperature, then concentrated under reduced pressure to give a residue. The residue was purified by Prep-HPLC (Condition 10, Gradient 4) to afford 4-[(3R)-3-aminopyrrolidin-1-yl]-N-{8-fluoro-2-methylimidazo[1,2-a]pyridin-6-yl}-2-methylindazole-7-carboxamide (10.4 mg, 23%) as a solid. LCMS (ES, m/z): 408[M+H]⁺. ¹H NMR (400 MHz, Methanol-d₄) δ 9.39 (d, J=1.4 Hz, 1H), 8.62 (s, 1H), 8.10 (d, J=8.3 Hz, 1H), 8.00 (s, 1H), 7.82 (d, J=11.3 Hz, 1H), 6.17 (d, J=8.4 Hz, 1H), 4.32 (s, 3H), 4.15 (s, 1H), 4.02 (dt, J=17.0, 9.3 Hz, 2H), 3.95-3.85 (m, 1H), 3.77 (d, J=10.9 Hz, 1H), 2.63-2.56 (m, 1H), 2.54 (s, 3H), 2.31 (s, 1H).

### Example 132: Synthesis of Compound 382

### Synthesis of Compound 382

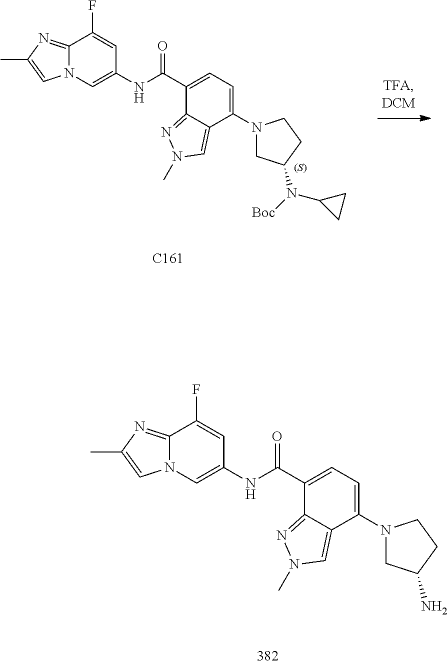

To a stirred solution of tert-butyl N-cyclopropyl-N-[(3S)-1-[7-({8-fluoro-2-methylimidazo[1,2-a]pyridin-6-yl}carbamoyl)-2-methylindazol-4-yl]pyrrolidin-3-yl]carbamate (60 mg, 0.110 mmol, 1 equiv) in DCM (1 mL, 143.58 equiv) was added TFA (1 mL, 122.88 equiv) at room temperature. The resulting mixture was stirred for 2 h at room temperature, then concentrated under reduced pressure to give a residue. The residue was purified by Prep-HPLC (Condition 10, Gradient 4) to afford 4-[(3S)-3-aminopyrrolidin-1-yl]-N-{8-fluoro-2-methylimidazo[1,2-a]pyridin-6-yl}-2-methylindazole-7-carboxamide (22.1 mg, 50%) as a solid. LCMS (ES, m/z): 408[M+H]⁺. ¹H NMR (400 MHz, Methanol-d₄) δ 9.39 (d, J=1.4 Hz, 1H), 8.62 (s, 1H), 8.10 (d, J=8.3 Hz, 1H), 8.00 (s, 1H), 7.82 (d, J=11.3 Hz, 1H), 6.17 (d, J=8.4 Hz, 1H), 4.32 (s, 3H), 4.15 (s, 1H), 4.02 (dt, J=17.0, 9.3 Hz, 2H), 3.95-3.85 (m, 1H), 3.77 (d, J=10.9 Hz, 1H), 2.63-2.56 (m, 1H), 2.54 (s, 3H), 2.31 (s, 1H).

### Example 133: Synthesis of Compound 383

### Synthesis of Intermediate C162

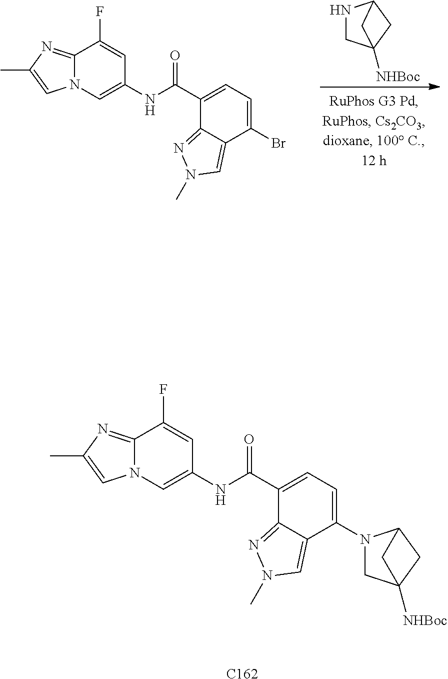 [SMILES: Cc1cn2cc(NC(=O)c3ccc(Br)c4cn(C)nc34)cc(F)c2n1 ; CC(C)(C)OC(=O)NC12CNC(C1)C2 ; Cc1cn2cc(NC(=O)c3ccc(N4CC5(NC(=O)OC(C)(C)C)CC4C5)c4cn(C)nc34)cc(F)c2n1 ; "C162"]

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-{2-azabicyclo[2.1.1]hexan-4-yl}carbamate (35.49 mg, 0.179 mmol, 1.2 equiv) in 1,4-dioxane (1.2 mL) were added Cs₂CO₃ (145.81 mg, 0.447 mmol, 3 equiv), RuPhos (13.92 mg, 0.030 mmol, 0.2 equiv) and RuPhos Palladacycle Gen.3 (12.48 mg, 0.015 mmol, 0.1 equiv) at room temperature under nitrogen atmosphere. The resulting mixture was stirred for 12 hr at 100° C. under nitrogen atmosphere. The resulting mixture was concentrated under reduced pressure. The residue was purified by silica gel column chromatography, eluted with CH₂Cl₂/MeOH (20:1) to afford tert-butyl N-{2-[7-({8-fluoro-2-methylimidazo[1,2-a]pyridin-6-yl}carbamoyl)-2-methylindazol-4-yl]-2-azabicyclo[2.1.1]hexan-4-yl}carbamate (C162, 70 mg, 90%) as a solid. LCMS (ES, m/z): 520 [M+H]⁺

### Synthesis of Compound 383

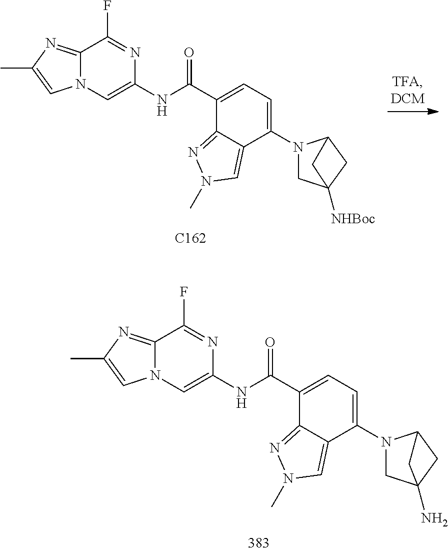

To a stirred solution of tert-butyl N-{2-[7-({8-fluoro-2-methylimidazo[1,2-a]pyridin-6-yl}carbamoyl)-2-methylindazol-4-yl]-2-azabicyclo[2.1.1]hexan-4-yl}carbamate (70 mg, 0.135 mmol, 1 equiv) in DCM (1 mL) was added TFA (1 mL, 13.463 mmol, 99.93 equiv) at room temperature. The resulting mixture was stirred for 1 h at room temperature. The resulting mixture was concentrated under reduced pressure. The crude product was purified by Prep-HPLC (Condition 10, Gradient 1) to afford 4-{4-amino-2-azabicyclo[2.1.1]hexan-2-yl}-N-{8-fluoro-2-methylimidazo[1,2-a]pyridin-6-yl}-2-methylindazole-7-carboxamide (Compound 383, 14 mg, 24%) as a solid. LCMS (ES, m/z): 420 [M+H]^{+ 1}H NMR (400 MHz, DMSO-d₆) δ 10.97 (s, 1H), 9.19 (s, 1H), 8.78 (s, 1H), 7.95-7.87 (m, 2H), 7.32 (d, J=12.4 Hz, 1H), 6.29 (d, J=8.4 Hz, 1H), 4.52 (s, 1H), 4.28 (s, 3H), 3.49 (s, 2H), 2.35 (s, 3H), 1.85 (s, 2H), 1.61 (s, 2H).

### Example 134: Synthesis of Compound 384

### Synthesis of Intermediate C163

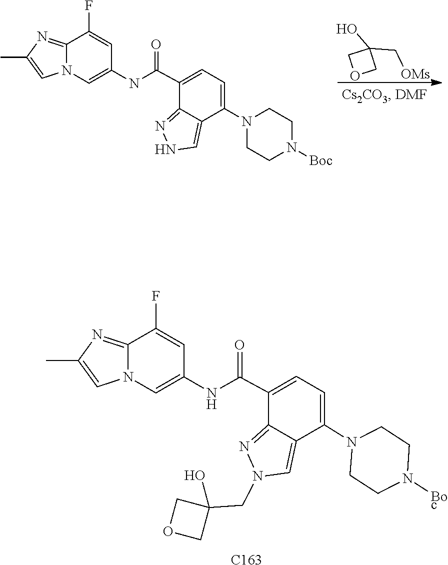

A solution of tert-butyl 4-[7-({8-fluoro-2-methylimidazo[1,2-a]pyridin-6-yl}carbamoyl)-2H-indazol-4-yl]piperazine-1-carboxylate (100 mg, 0.203 mmol, 1 equiv), (3-hydroxyoxetan-3-yl)methyl methanesulfonate (55 mg, 0.304 mmol, 1.5 equiv) and Cs₂CO₃ (198 mg, 0.609 mmol, 3.0 equiv) in DMF (2 mL) was stirred for 12 hr at 90° C. The resulting mixture was concentrated under reduced pressure. The residue was purified by reverse flash chromatography (Condition 3, Gradient 1) to afford tert-butyl 4-[7-({8-fluoro-2-methylimidazo[1,2-a]pyridin-6-yl}carbamoyl)-2-[(3-hydroxyoxetan-3-yl)methyl]indazol-4-yl]piperazine-1-carboxylate (C163, 35 mg, 29%) as a solid. LCMS (ES, m/z): 580 [M+H]⁺

### Synthesis of Compound 384

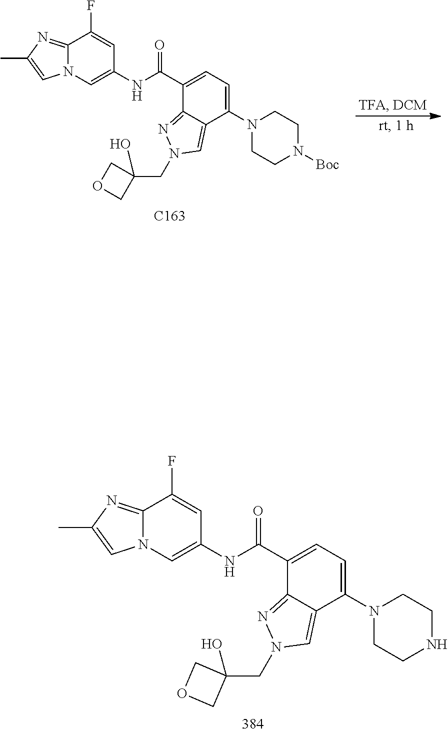

A solution of tert-butyl 4-[7-({8-fluoro-2-methylimidazo[1,2-a]pyridin-6-yl}carbamoyl)-2-[(3-hydroxyoxetan-3-yl)methyl]indazol-4-yl]piperazine-1-carboxylate (30 mg, 0.052 mmol, 1 equiv) and TFA (0.2 mL) in DCM (2 mL) was stirred for 1 h at room temperature. The resulting mixture was concentrated under reduced pressure. The residue was purified by reverse flash chromatography (Condition 3, Gradient 2) to afford N-{8-fluoro-2-methylimidazo[1,2-a]pyridin-6-yl}-2-[(3-hydroxyoxetan-3-yl)methyl]-4-(piperazin-1-yl) indazole-7-carboxamide (Compound 384, 10 mg, 40%) as a solid. LCMS (ES, m/z): 480 [M+H]^{+ 1}H NMR (300 MHz, DMSO-d₆) δ 11.03 (s, 1H), 9.24 (d, J=1.6 Hz, 1H), 8.77 (s, 1H), 8.00 (d, J=8.1 Hz, 1H), 7.92 (d, J=3.2 Hz, 1H), 7.25 (dd, J=12.4, 1.7 Hz, 1H), 6.50 (d, J=8.2 Hz, 1H), 6.25 (s, 1H), 4.88 (s, 2H), 4.84-4.70 (m, 2H), 4.51 (d, J=6.5 Hz, 2H), 3.45-3.37 (m, 4H), 2.91-2.73 (m, 4H), 2.35 (s, 3H).

### Example 135: Synthesis of Compound 385

### Synthesis of Intermediate C164

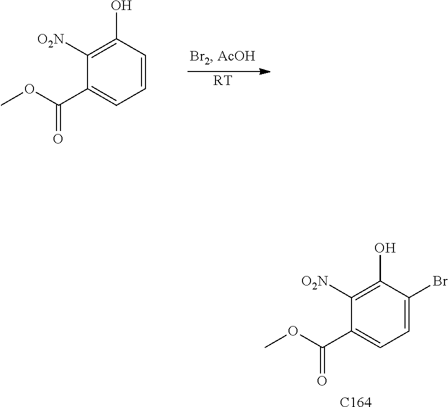

Into a 40 mL round-bottom flask were added methyl 3-hydroxy-2-nitrobenzoate (700 mg, 3.551 mmol, 1 equiv), acetic acid (10 mL) and Br₂ (851.14 mg, 5.327 mmol, 1.5 equiv) at 25 degrees C. The resulting mixture was stirred for 12 hr at 25° C. The resulting mixture was concentrated under reduced pressure. The residue was purified by silica gel column chromatography, eluted with PE/EA (3:1) to afford methyl 4-bromo-3-hydroxy-2-nitrobenzoate (C164, 730 mg, 74%) as a solid. LCMS (ES, m/z): 274 [M−H]⁻

### Synthesis of Intermediate C165

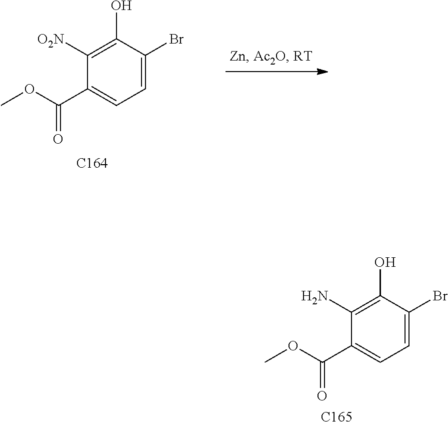

Into a 40 mL round-bottom flask were added methyl 4-bromo-3-hydroxy-2-nitrobenzoate (300 mg, 1.087 mmol, 1 equiv), acetic acid (6 mL) and zinc (213.16 mg, 3.260 mmol, 3.00 equiv) at 25 degrees C. The resulting mixture was stirred for 12 h at 25 degrees C. The reaction was quenched by the addition of sodium bicarbonate aqueous solution (50 mL) at 25 degrees C. The aqueous layer was extracted with ethyl acetate (3×20 mL). The resulting mixture was concentrated under reduced pressure. The residue was purified by silica gel column chromatography, eluted with PE/EA (1:1) to afford methyl 2-amino-4-bromo-3-hydroxybenzoate (C165, 200 mg, 74%) as a solid. LCMS (ES, m/z): 246 [M+H]⁺

### Synthesis of Intermediate C166

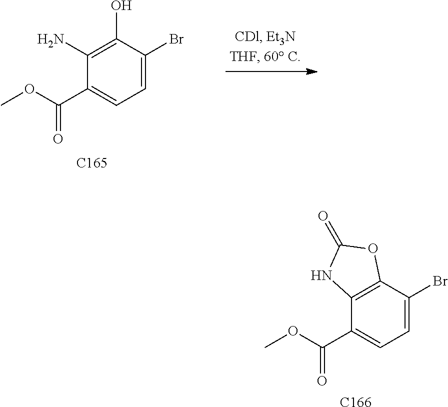

Into a 40 mL round-bottom flask were added methyl 2-amino-4-bromo-3-hydroxybenzoate (200 mg, 0.813 mmol, 1 equiv) THF (10 mL) carbonyldiimidazole (197.70 mg, 1.220 mmol, 1.5 equiv) and triethylamine (246.75 mg, 2.439 mmol, 3 equiv) at 25° C. The resulting mixture was stirred for 12 h at 60° C. The resulting mixture was concentrated under reduced pressure. The residue was purified by silica gel column chromatography, eluted with PE/EA (1:1) to afford methyl 7-bromo-2-oxo-3H-1,3-benz oxazole-4-carboxylate (C166, 200 mg, 90%) as a solid. LCMS (ES, m/z): 272 [M−H]⁻

### Synthesis of Intermediate C167

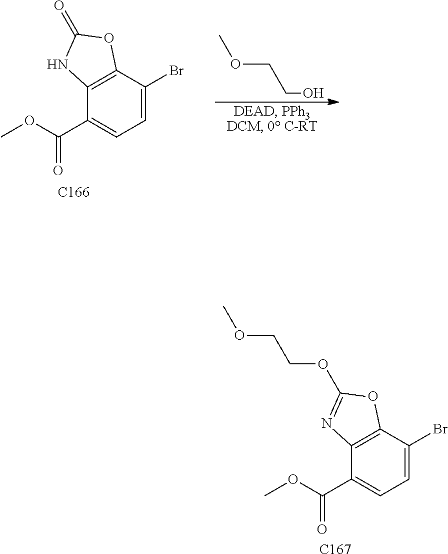

Into a 40 mL vial were added methyl 7-bromo-2-oxo-3H-1,3-benzoxazole-4-carboxylate (200 mg, 0.735 mmol, 1 equiv), triphenylphosphine (289.24 mg, 1.103 mmol, 1.5 equiv), DCM (4 mL) and 2-methoxyethanol (67.13 mg, 0.882 mmol, 1.2 equiv) at room temperature. The resulting mixture was stirred for 1 hr at 0° C. under nitrogen atmosphere. To the above mixture was added DEAD (192.05 mg, 1.103 mmol, 1.5 equiv). The resulting mixture was stirred for additional 2 h at 25 degrees C. The reaction was quenched by the addition of water (10 mL) at room temperature. The aqueous layer was extracted with ethyl acetate (2×10 mL). The resulting liquid was dried Na₂SO₄. The resulting mixture was concentrated under reduced pressure. The residue was purified by silica gel column chromatography, eluted with PE/EA (3:1) to afford methyl 7-bromo-2-(2-methoxyethoxy)-1,3-benzoxazole-4-carboxylate (C167, 160 mg, 65%) as a solid.

LCMS (ES, m/z): 330 [M+H]⁺

### Synthesis of Intermediate C168

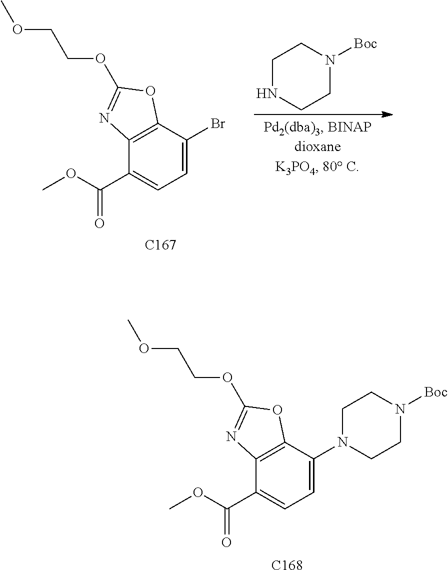

Into a 40 mL vial were added methyl 7-bromo-2-(2-methoxyethoxy)-1,3-benzoxazole-4-carboxylate (150 mg, 0.454 mmol, 1 equiv), tert-butyl piperazine-1-carboxylate (101.55 mg, 0.545 mmol, 1.2 equiv) BINAP (56.59 mg, 0.091 mmol, 0.2 equiv), K₃PO₄ (289.33 mg, 1.362 mmol, 3 equiv), Pd₂(dba)₃ (41.61 mg, 0.045 mmol, 0.1 equiv) and dioxane (5 mL) at room temperature. The resulting mixture was stirred for 3 hr at 80° C. under nitrogen atmosphere. The resulting mixture was concentrated under reduced pressure. The residue was purified by silica gel column chromatography, eluted with THF/EA (1:1) to afford methyl 7-[4-(tert-butoxycarbonyl)piperazin-1-yl]-2-(2-methoxyethoxy)-1,3-benzoxazole-4-carboxylate (C168, 120 mg, 60%) as a solid. LCMS (ES, m/z): 436 [M+H]⁺

### Synthesis of Intermediate C169

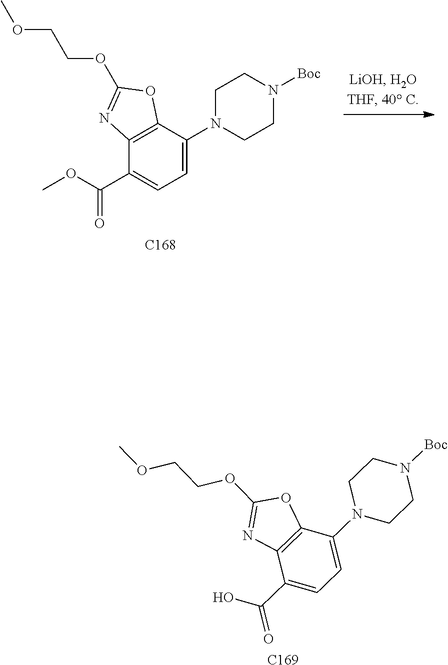

Into a 40 mL vial were added methyl 7-[4-(tert-butoxycarbonyl)piperazin-1-yl]-2-(2-methoxyethoxy)-1,3-benzoxazole-4-carboxylate (120 mg, 0.276 mmol, 1 equiv), tetrahydrofuran (2 mL), water (2 mL) and LiOH (9.90 mg, 0.414 mmol, 1.5 equiv) at room temperature. The resulting mixture was stirred for 3 h at 40° C. The resulting mixture was diluted with water (30 mL). The mixture was acidified to pH 5 with HCl (aq.). The aqueous layer was extracted with ethyl acetate (2×50 mL). The resulting mixture was concentrated under reduced pressure. This resulted in 7-[4-(tert-butoxycarbonyl)piperazin-1-yl]-2-(2-methoxy ethoxy)-1,3-benzoxazole-4-carboxylic acid (C169, 100 mg, 86%) as a solid. LCMS (ES, m/z): 422 [M+H]⁺

### Synthesis of Intermediate C170

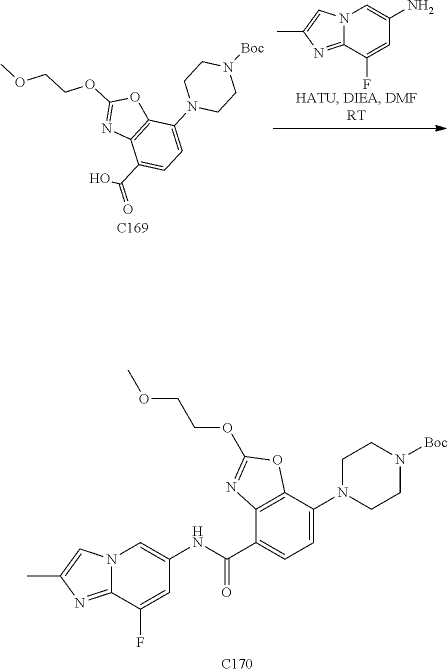

Into a 40 mL vial were added 7-[4-(tert-butoxycarbonyl)piperazin-1-yl]-2-(2-methoxyethoxy)-1,3-benzoxazole-4-carboxylic acid (100 mg, 0.237 mmol, 1 equiv), 8-fluoro-2-methylimidazo[1,2-a]pyridin-6-amine (47.03 mg, 0.284 mmol, 1.2 equiv), HATU (85.79 mg, 0.355 mmol, 1.5 equiv), dimethylformamide (3 mL) and DIEA (92.00 mg, 0.711 mmol, 3 equiv) at room temperature. The resulting mixture was stirred for 2 hr at room temperature. The reaction was quenched by the addition of water (10 mL) at room temperature. The aqueous layer was extracted with ethyl acetate (2×10 mL). The resulting liquid was dried over Na₂SO₄. The resulting mixture was concentrated under reduced pressure. The residue was purified by silica gel column chromatography, eluted with PE/EA (3:1) to afford tert-butyl 4-[4-({8-fluoro-2-methylimidazo [1,2-a]pyri din-6-yl}carbamoyl)-2-(2-methoxyethoxy)-1,3-benzoxazol-7-yl]piperazine-1-carboxylate (C170, 63 mg, 46%) as a solid. LCMS (ES, m/z): 569 [M+H]⁺

### Synthesis of Compound 367

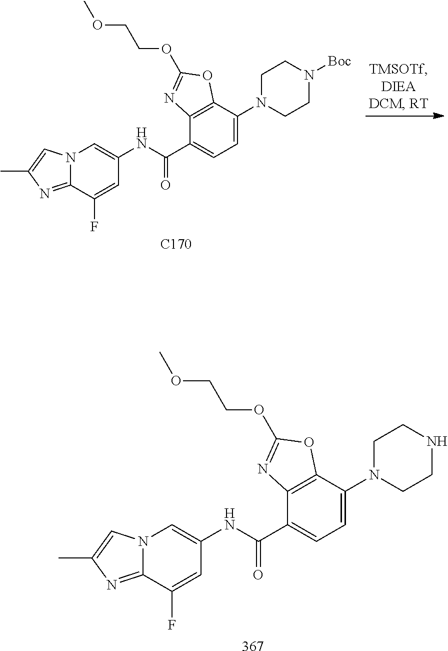

Into a 8 mL vial were added tert-butyl 4-[4-({8-fluoro-2-methylimidazo[1,2-a]pyridin-6-yl}carbamoyl)-2-(2-methoxyethoxy)-1,3-benzoxazol-7-yl]piperazine-1-carboxylate (40 mg, 0.070 mmol, 1 equiv), DCM (1 mL), trimethylsilyl triflate (156.35 mg, 0.700 mmol, 10 equiv) and DIEA (90.92 mg, 0.700 mmol, 10 equiv) at room temperature. The resulting mixture was stirred for 1 h at room temperature. The crude product was purified by Prep-HPLC (Condition 11, Gradient 1) to afford N-{8-fluoro-2-methylimidazo[1,2-a]pyridin-6-yl}-2-(2-methoxyethoxy)-7-(piperazin-1-yl)-1,3-benzoxazole-4-carboxamide (Compound 367, 7.7 mg, 23%) as a solid. LCMS (ES, m/z): 469 [M+H]⁺ 1H NMR (300 MHz, DMSO-d6) δ 10.62 (s, 1H), 9.06 (d, J=1.6 Hz, 1H), 7.94 (d, J=3.1 Hz, 1H), 7.40 (d, J=8.7 Hz, 1H), 7.22 (dd, J=12.6, 1.6 Hz, 1H), 6.79 (d, J=8.8 Hz, 1H), 4.20 (t, J=5.2 Hz, 2H), 3.45 (t, J=5.2 Hz, 2H), 3.21 (dd, J=6.4, 3.5 Hz, 4H), 3.09 (s, 3H), 2.88 (dd, J=6.2, 3.5 Hz, 4H), 2.34 (s, 2H).

### Example 136: Synthesis of Compound 386

### Synthesis of Intermediate C171

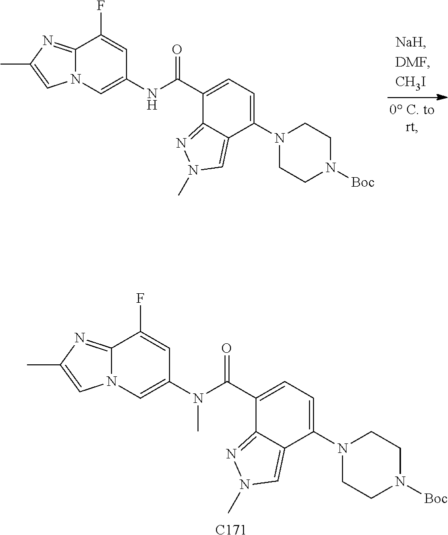 [SMILES: Cc1cn2cc(NC(=O)c3ccc(N4CCN(C(=O)OC(C)(C)C)CC4)c4cn(C)nc34)cc(F)c2n1 ; Cc1cn2cc(N(C)C(=O)c3ccc(N4CCN(C(=O)OC(C)(C)C)CC4)c4cn(C)nc34)cc(F)c2n1 ; "C171"]

To a solution of tert-butyl 4-[7-({8-fluoro-2-methylimidazo[1,2-a]pyridin-6-yl}carbamoyl)-2-methylindazol-4-yl]piperazine-1-carboxylate (50 mg, 0.099 mmol, 1 equiv) in DMF (1 mL) was added sodium hydride (60% in oil, 7.88 mg, 2 equiv) at 0° C. The mixture was stirred for 15 min. CH₃I (13.28 mg, 0.094 mmol, 0.95 equiv) was added and the mixture was allowed to warm to room temperature and stirred for 2 hr. The reaction mixture was quenched by water and extracted with DCM (3×25 mL). The organic phase was concentrated under reduced pressure to afford tert-butyl 4-[7-({8-fluoro-2-methylimidazo[1,2-a]pyridin-6-yl}(methyl)carbamoyl)-2-methylindazol-4-yl]piperazine-1-carboxylate (C171, 40 mg, 77%) as a solid. LCMS (ES, m/z): 522 [M+H]⁺

### Synthesis of Compound 386

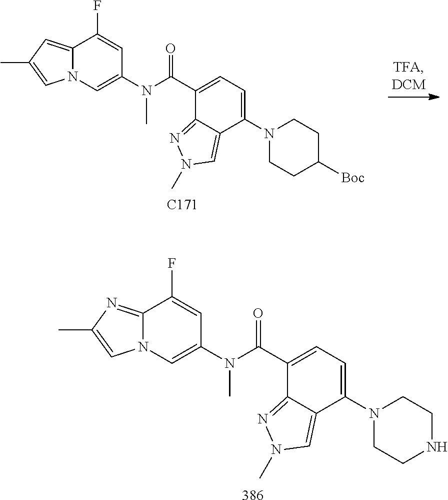

To a stirred solution of tert-butyl 4-[7-({8-fluoro-2-methylimidazo[1,2-a]pyridin-6-yl}(methyl)carbamoyl)-2-methylindazol-4-yl]piperazine-1-carboxylate (40 mg, 0.077 mmol, 1 equiv) in DCM (1 mL) was added TFA (1 mL) at room temperature. The resulting mixture was stirred for 2 hr at room temperature. The resulting mixture was concentrated under reduced pressure. The crude product was purified by Prep-HPLC (Condition 10, Gradient 2) to afford N-{8-fluoro-2-methylimidazo[1,2-a]pyridin-6-yl}-N,2-dimethyl-4-(piperazin-1-yl) indazole-7-carboxamide (Compound 386, 8.8 mg, 27%) as a solid. LCMS (ES, m/z): 422 [M+H]^{+ 1}H NMR (400 MHz, DMSO-d₆) δ 8.38 (s, 1H), 8.28 (s, 1H), 7.65 (d, J=2.9 Hz, 1H), 7.19-7.11 (m, 2H), 6.22 (d, J=7.6 Hz, 1H), 4.08 (s, 3H), 3.35 (s, 3H), 3.14 (t, J=4.9 Hz, 4H), 2.93 (t, J=4.9 Hz, 4H), 2.26 (s, 3H).

### Example 137: Synthesis of Compound 387

### Synthesis of Intermediate C172

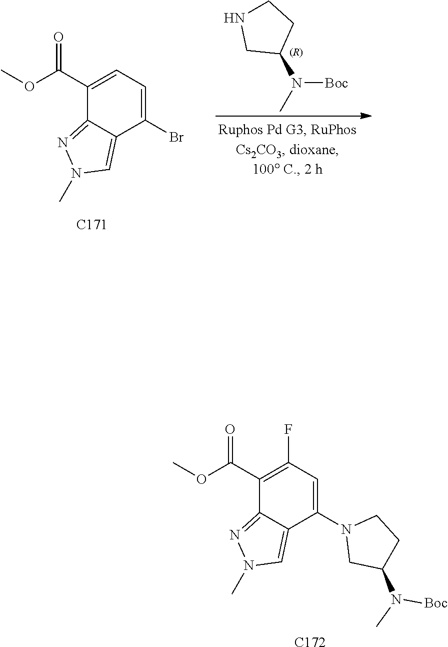

To a stirred solution of methyl 4-bromo-2-methylindazole-7-carboxylate (350 mg, 1.30 mmol, 1.0 equiv) and tert-butyl N-methyl-N-[(3R)-pyrrolidin-3-yl]carbamate (286.5 mg, 1.43 mmol, 1.1 equiv) in dioxane (4 mL) were added Cs₂CO₃ (1.27 g, 3.90 mmol, 3.0 equiv), RuPhos (121.3 mg, 0.26 mmol, 0.2 equiv) and RuPhos Palladacycle Gen.3 (108.7 mg, 0.13 mmol, 0.1 equiv) at room temperature under nitrogen atmosphere. The resulting mixture was stirred for 2 hr at 100° C. under nitrogen atmosphere. The resulting mixture was concentrated under reduced pressure. The residue was purified by silica gel column chromatography, eluted with CH₂Cl₂/MeOH (10:1) to afford methyl 4-[(3R)-3-[(tert-butoxycarbonyl)(methyl)amino]pyrrolidin-1-yl]-2-methylindazole-7-carboxylate (C172, 398 mg, 78%) as a solid. LCMS (ES, m/z): 389 [M+H]⁺

### Synthesis of Intermediate C173

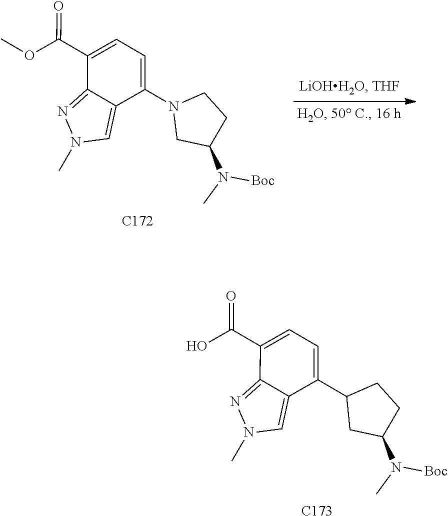 [SMILES: COC(=O)c1ccc(N2CC[C@@H](N(C)C(=O)OC(C)(C)C)C2)c2cn(C)nc12 ; "C172" ; CN(C(=O)OC(C)(C)C)[C@@H]1CCC(c2ccc(C(=O)O)c3nn(C)cc23)C1 ; "C173"]

To a stirred solution of methyl 4-[(3R)-3-[(tert-butoxycarbonyl)(methyl)amino]pyrrolidin-1-yl]-2-methylindazole-7-carboxylate (398.0 mg, 1.02 mmol, 1.0 equiv) in THF (4 mL) were added H₂O (4 mL) and LiOH (214.9 mg, 5.12 mmol, 5.0 equiv) at room temperature. The resulting mixture was stirred for 16 hr at 50° C. The resulting mixture was concentrated under vacuum and diluted with water (10 mL). The mixture was acidified to pH 7 with HCl (2 M) and was extracted with ethyl acetate (3×10 mL). The combined organic layers were washed with brine (3×10 mL), dried over anhydrous Na₂SO₄. After filtration, the filtrate was concentrated under reduced pressure to afford 4-[(3R)-3-[(tert-butoxycarbonyl)(methyl)amino]pyrrolidin-1-yl]-2-methylindazole-7-carboxylic acid (C173, 312 mg, 81%) as a solid. LCMS (ES, m/z): 375 [M+H]⁺

### Synthesis of Intermediate C174

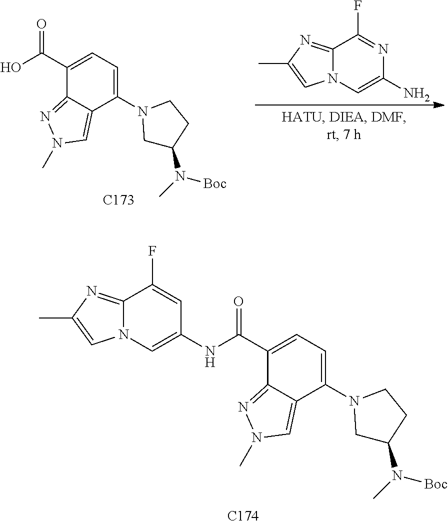 [SMILES: CN(C(=O)OC(C)(C)C)[C@@H]1CCN(c2ccc(C(=O)O)c3nn(C)cc23)C1 ; Cc1cn2cc(N)nc(F)c2n1 ; "C173" ; Cc1cn2cc(NC(=O)c3ccc(N4CC[C@@H](N(C)C(=O)OC(C)(C)C)C4)c4cn(C)nc34)cc(F)c2n1 ; "C174"]

To a stirred solution of 4-[(3R)-3-[(tert-butoxycarbonyl)(methyl)amino]pyrrolidin-1-yl]-2-methylindazole-7-carboxylic acid (312.0 mg, 0.83 mmol, 1.0 equiv) and 8-fluoro-2-methylimidazo[1,2-a]pyrazin-6-amine (152.3 mg, 0.91 mmol, 1.1 equiv) in DMF (4 mL) were added HATU (475.2 mg, 1.24 mmol, 1.5 equiv) and DIEA (323.0 mg, 2.49 mmol, 3.0 equiv) at room temperature. The resulting mixture was stirred for 7 hr at room temperature. The resulting mixture was diluted with water (5 mL) and extracted with ethyl acetate (3×20 mL). The combined organic layers were washed with brine (1×10 mL), dried over anhydrous Na₂SO₄. After filtration, the filtrate was concentrated under reduced pressure. The residue was purified by silica gel column chromatography, eluted with ethyl acetate (100%) to afford tert-butyl N-[(3R)-1-[7-({8-fluoro-2-methylimidazo[1,2-a]pyridin-6-yl}carbamoyl)-2-methylindazol-4-yl]pyrrolidin-3-yl]-N-methylcarbamate (C174, 210 mg, 48%) as a solid. LCMS (ES, m/z): 522 [M+H]⁺

### Synthesis of Compound 387

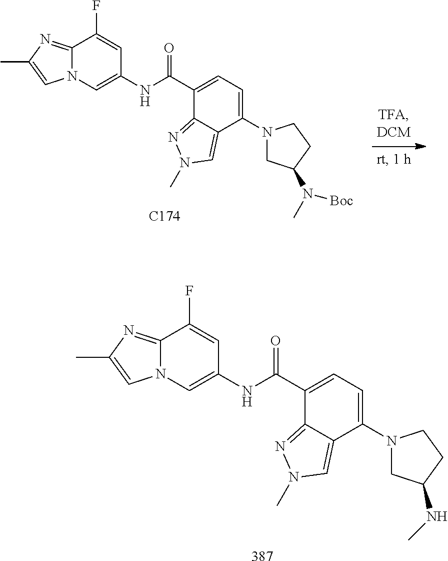 [SMILES: "C174" ; Cc1cn2cc(NC(=O)c3ccc(N4CC[C@@H](N(C)C(=O)OC(C)(C)C)C4)c4cn(C)nc34)cc(F)c2n1 ; CN[C@@H]1CCN(c2ccc(C(=O)Nc3cc(F)c4nc(C)cn4c3)c3nn(C)cc23)C1 ; "387"]

To a stirred solution of tert-butyl N-[(3R)-1-[7-({8-fluoro-2-methylimidazo[1,2-a]pyridin-6-yl}carbamoyl)-2-methylindazol-4-yl]pyrrolidin-3-yl]-N-methylcarbamate (210 mg, 0.40 mmol, 1.0 equiv) in DCM (3 mL) was added TFA (1 mL) dropwise at 0° C. The resulting mixture was stirred for 1 hr at room temperature. The resulting mixture was concentrated under vacuum. The crude product was purified by Prep-HPLC (Condition 10, Gradient 1) to afford N-{8-fluoro-2-methylimidazo[1,2-a]pyridin-6-yl}-2-methyl-4-[(3R)-3-(methylamino)pyrrolidin-1-yl]indazole-7-carboxamide (Compound 387, 32.4 mg, 19%) as a solid. LCMS (ES, m/z): 422 [M+H]^{+ 1}H NMR (400 MHz, DMSO-d₆) δ 11.01 (s, 1H), 9.19 (d, J=1.7 Hz, 1H), 8.83 (s, 1H), 8.11-7.79 (m, 2H), 7.31 (dd, J=12.4, 1.7 Hz, 1H), 6.02 (d, J=8.4 Hz, 1H), 4.27 (s, 3H), 3.76 (t, J=15.2, 6.5 Hz, 2H), 3.66 (s, 1H), 3.43 (d, J=10.8 Hz, 1H), 3.36 (d, J=5.4 Hz, 1H), 2.35 (d, J=1.5 Hz, 6H), 2.15 (dt, J=12.8, 6.5 Hz, 1H), 1.93 (dd, J=12.0, 6.3 Hz, 1H).

### Example 138: Synthesis of Compound 388

### Synthesis of Intermediate C175

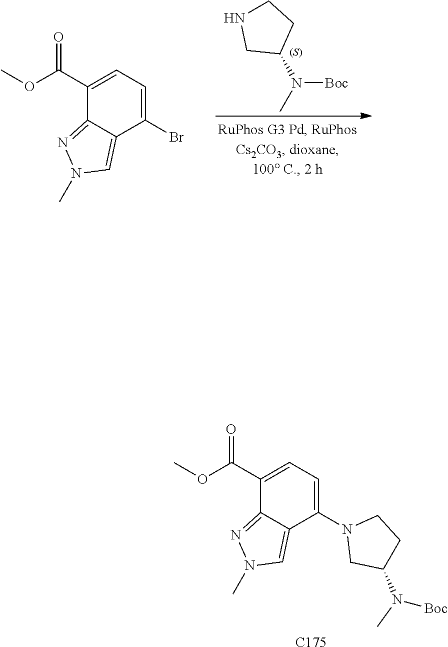

To a stirred mixture of methyl 4-bromo-2-methylindazole-7-carboxylate (400 mg, 1.486 mmol, 1 equiv) and tert-butyl N-methyl-N-[(3R)-pyrrolidin-3-yl]carbamate (357.3 mg, 1.783 mmol, 1.2 equiv) in dioxane (8 mL) were added Cs₂CO₃ (1.45 g, 4.458 mmol, 3.0 equiv) and RuPhos (138.7 mg, 0.297 mmol, 0.2 equiv) and RuPhos Palladacycle Gen.3 (124.3 mg, 0.149 mmol, 0.1 equiv) at room temperature under nitrogen atmosphere. The resulting mixture was stirred for 2 hr at 100° C. under nitrogen atmosphere. The resulting mixture was concentrated under vacuum. The residue was purified by silica gel column chromatography, eluted with PE/EA (1:1) to afford methyl (S)-4-(3-((tert-butoxycarbonyl)(methyl)amino)pyrrolidin-1-yl)-2-methyl-2H-indazole-7-carboxylate (C175, 650 mg, 112%) as a solid. LCMS (ES, m/z): 389 [M+H]⁺

### Synthesis of Intermediate C176

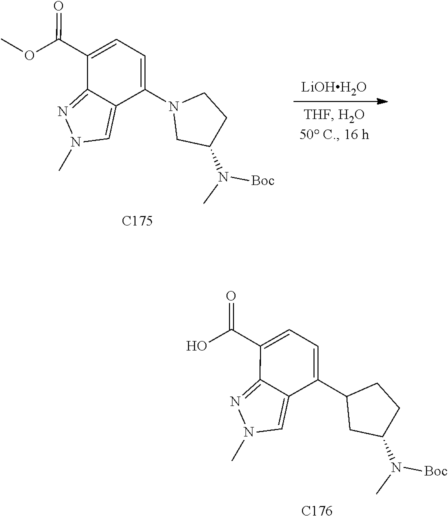 [SMILES: COC(=O)c1ccc(N2CC[C@H](N(C)C(=O)OC(C)(C)C)C2)c2cn(C)nc12 ; "LiOH·H2O" ; "C175" ; CN(C(=O)OC(C)(C)C)[C@H]1CCC(c2ccc(C(=O)O)c3nn(C)cc23)C1 ; "C176"]

To a stirred mixture of methyl (S)-4-(3-((tert-butoxycarbonyl)(methyl)amino)pyrrolidin-1-yl)-2-methyl-2H-indazole-7-carboxylate (650.0 mg, 1.673 mmol, 1 equiv) in THF (8 mL) were added H₂O (8 mL) and lithium hydroxide hydrate (561.7 mg, 13.384 mmol, 8.0 equiv) at room temperature. The resulting mixture was stirred for 16 hr at 50° C. The resulting mixture was concentrated under vacuum. The resulting mixture was diluted with water (10 mL). The mixture was acidified to pH 6 with HCl (2 M). The resulting mixture was extracted with ethyl acetate (3×10 mL). The combined organic layers were washed with water (2×10 mL), dried over anhydrous Na₂SO₄. After filtration, the filtrate was concentrated under reduced pressure to afford (S)-4-(3-((tert-butoxycarbonyl)(methyl)amino)pyrrolidin-1-yl)-2-methyl-2H-indazole-7-carboxylic acid (C176, 570 mg, 90%) as a solid. LCMS (ES, m/z): 375 [M+H]⁺

### Synthesis of Intermediate C177

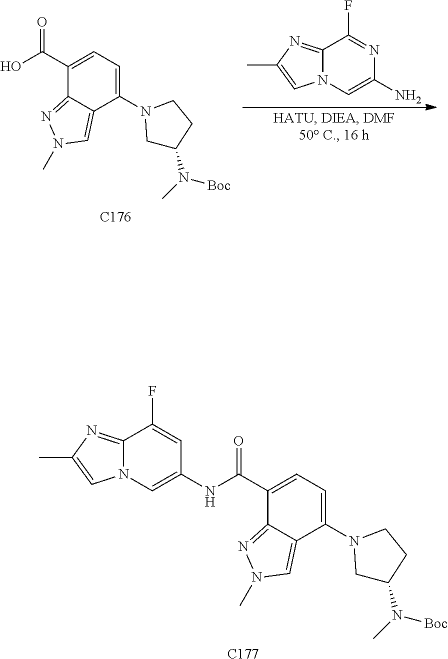

To a stirred mixture of (S)-4-(3-((tert-butoxycarbonyl)(methyl)amino)pyrrolidin-1-yl)-2-methyl-2H-indazole-7-carboxylic acid (200.0 mg, 0.534 mmol, 1.0 equiv) and 8-fluoro-2-methylimidazo[1,2-a]pyridin-6-amine (132.3 mg, 0.801 mmol, 1.5 equiv) in DMF (2.5 mL) were added DIEA (276.1 mg, 2.136 mmol, 4.0 equiv) and HATU (243.7 mg, 0.641 mmol, 1.2 equiv) at room temperature. The resulting mixture was stirred for 16 hr at 50° C. The mixture was allowed to cool down to room temperature. The resulting mixture was diluted with water (10 mL). The resulting mixture was extracted with ethyl acetate (3×10 mL). The combined organic layers were washed with water (3×10 mL), dried over anhydrous Na₂SO₄. After filtration, the filtrate was concentrated under reduced pressure. The residue was purified by silica gel column chromatography, eluted with EA to afford tert-butyl (S)-(1-(7-((8-fluoro-2-methylimidazo[1,2-a]pyridin-6-yl)carbamoyl)-2-methyl-2H-indazol-4-yl)pyrrolidin-3-yl)(methyl)carbamate (C177, 130 mg, 46%) as a solid. LCMS (ES, m/z): 522 [M+H]⁺

### Synthesis of Compound 388

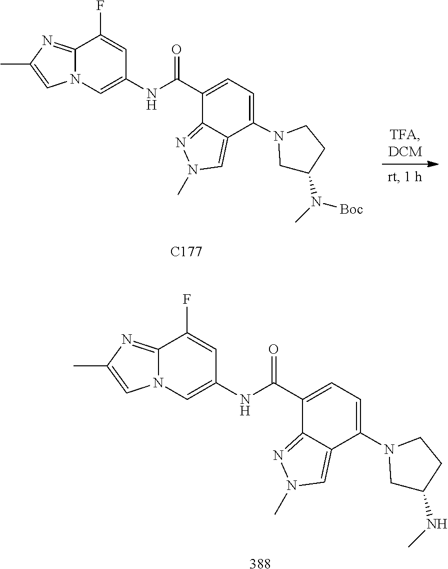 [SMILES: Cc1cn2cc(NC(=O)c3ccc(N4CC[C@H](N(C)C(=O)OC(C)(C)C)C4)c4cn(C)nc34)cc(F)c2n1 ; "C177" ; CN[C@H]1CCN(c2ccc(C(=O)Nc3cc(F)c4nc(C)cn4c3)c3nn(C)cc23)C1 ; "388"]

To a stirred mixture of tert-butyl (S)-(1-(7-((8-fluoro-2-methylimidazo[1,2-a]pyridin-6-yl)carbamoyl)-2-methyl-2H-indazol-4-yl)pyrrolidin-3-yl)(methyl)carbamate (110 mg, 0.211 mmol, 1 equiv) in DCM (2.5 mL) was added TFA (0.5 mL) dropwise at room temperature. The resulting mixture was stirred for 1 h at room temperature. The resulting mixture was concentrated under vacuum. The crude product was purified by Prep-HPLC (Condition 10, Gradient 1) to afford (S)—N-(8-fluoro-2-methylimidazo[1,2-a]pyridin-6-yl)-2-methyl-4-(3-(methylamino)pyrrolidin-1-yl)-2H-indazole-7-carboxamide (Compound 388, 55 mg, 61%) as a solid. LCMS (ES, m/z): 422 [M+H]^{+ 1}H NMR (400 MHz, DMSO-d₆) δ 11.01 (s, 1H), 9.19 (d, J=1.6 Hz, 1H), 8.82 (s, 1H), 7.96-7.86 (m, 2H), 7.29 (dd, J=12.4, 1.7 Hz, 1H), 6.01 (d, J=8.4 Hz, 1H), 4.27 (t, 3H), 3.75 (dq, J=21.5, 7.5, 6.8 Hz, 2H), 3.64 (d, J=7.5 Hz, 1H), 3.42 (dd, J=10.6, 4.0 Hz, 1H), 3.35 (d, J=10.5 Hz, 1H), 2.35 (s, 6H), 2.21-2.11 (m, 1H), 1.92 (dq, J=12.3, 6.2 Hz, 1H).

### Example 139: Synthesis of Compound 389

### Synthesis of Intermediate C178

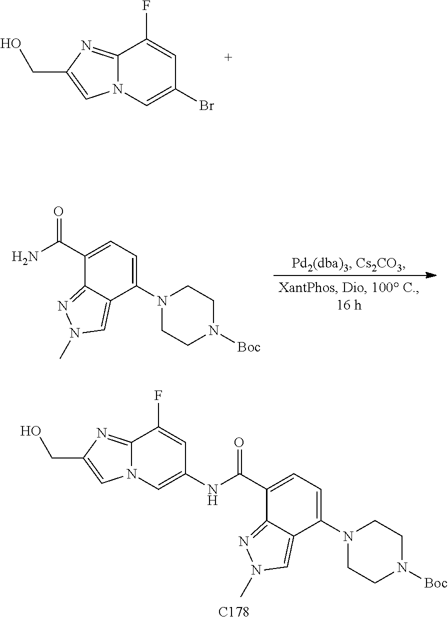

To a stirred solution of {6-bromo-8-fluoroimidazo[1,2-a]pyridin-2-yl}methanol (60 mg, 0.245 mmol, 1 equiv) and tert-butyl 4-(7-carbamoyl-2-methylindazol-4-yl)piperazine-1-carboxylate (88.01 mg, 0.245 mmol, 1 equiv) and Cs₂CO₃ (239.33 mg, 0.735 mmol, 3.0 equiv) in Dioxane (2 mL) were added XantPhos (14.17 mg, 0.025 mmol, 0.1 equiv) and Pd₂(dba)₃ (11.21 mg, 0.012 mmol, 0.05 equiv) at room temperature under nitrogen atmosphere. The resulting mixture was stirred for 16 hr at 100° C. under nitrogen atmosphere. The residue was purified by reverse flash chromatography (Condition 3, Gradient 3) to afford tert-butyl 4-(7-{[8-fluoro-2-(hydroxymethyl)imidazo[1,2-a]pyridin-6-yl]carbamoyl}-2-methylindazol-4-yl)piperazine-1-carboxylate (C178, 40 mg, 31%) as a solid. LCMS (ES, m/z): 524 [M+H]⁺

### Synthesis of Compound 389

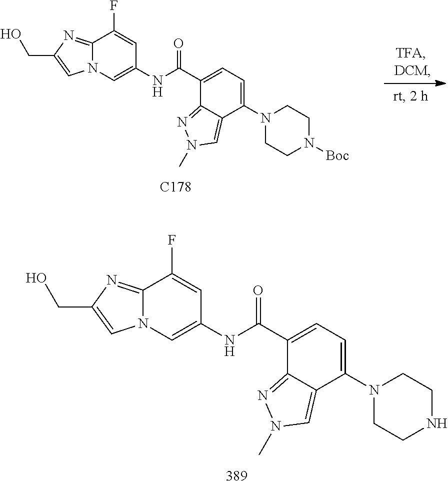

A mixture of tert-butyl 4-(7-{[8-fluoro-2-(hydroxymethyl)imidazo[1,2-a]pyridin-6-yl]carbamoyl}-2-methylindazol-4-yl)piperazine-1-carboxylate (35 mg, 0.067 mmol, 1 equiv) in DCM (2 mL) and TFA (1 mL) was stirred for 2 hr at room temperature. The resulting mixture was concentrated under reduced pressure. The residue was purified by reverse flash chromatography (Condition 4, Gradient 1) to afford N-[8-fluoro-2-(hydroxymethyl)imidazo[1,2-a]pyridin-6-yl]-2-methyl-4-(piperazin-1-yl) indazole-7-carboxamide; trifluoroacetic acid (Compound 389, 18 mg, 50%) as a solid. LCMS (ES, m/z): 424 [M+H]^{+ 1}H NMR (400 MHz, DMSO-d₆) δ 11.12 (s, 1H), 9.35 (s, 1H), 8.89-8.82 (m, 3H), 8.10 (s, 1H), 8.03-8.01 (d, J=8 Hz, 1H), 7.54-7.53 (br, 1H), 6.62-6.60 (d, J=8 Hz, 1H), 4.66 (s, 2H), 4.32 (s, 3H), 3.60-3.52 (m, 4H), 3.40-3.25 (m, 4H).

### Example 140: Synthesis of Compound 387

### Synthesis of Compound 387

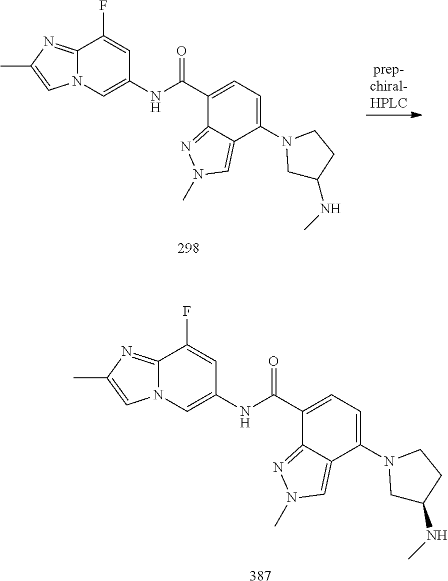 [SMILES: CNC1CCN(c2ccc(C(=O)Nc3cc(F)c4nc(C)cn4c3)c3nn(C)cc23)C1 ; "298" ; CN[C@@H]1CCN(c2ccc(C(=O)Nc3cc(F)c4nc(C)cn4c3)c3nn(C)cc23)C1 ; "387"]

Compound 298 was separated by prep-chiral-HPLC (Condition 2, Gradient 1) to yield (R)—N-(8-fluoro-2-methylimidazo[1,2-a]pyridin-6-yl)-2-methyl-4-(3-(methylamino)pyrrolidin-1-yl)-2H-indazole-7-carboxamide (5.9 mg, 25%) as a solid. LCMS (ES, m/z): 422 [M+H]^{+ 1}H NMR (400 MHz, Methanol-d₄) δ 9.00 (s, 1H), 8.53 (s, 1H), 8.03 (d, J=8.3 Hz, 1H), 7.70 (d, J=3.0 Hz, 1H), 7.13 (d, J=12.4 Hz, 1H), 6.07 (d, J=8.4 Hz, 1H), 4.30 (s, 3H), 3.90-3.77 (m, 2H), 3.73-3.67 (m, 1H), 3.53-3.52 (m, 2H), 2.53 (s, 3H), 2.44 (s, 3H), 2.41-2.33 (m, 1H), 2.09-2.03 (m, 1H).

### Example 141: Synthesis of Compound 388

### Synthesis of Compound 388

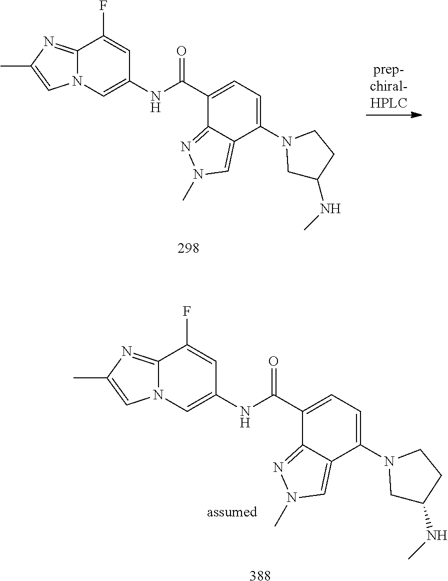 [SMILES: CNC1CCN(c2ccc(C(=O)Nc3cc(F)c4nc(C)cn4c3)c3nn(C)cc23)C1 ; "298" ; CN[C@H]1CCN(c2ccc(C(=O)Nc3cc(F)c4nc(C)cn4c3)c3nn(C)cc23)C1 ; "388"]

Compound 298 was separated by prep-chiral-HPLC (Condition 2, Gradient 1) to yield (S)—N-(8-fluoro-2-methylimidazo[1,2-a]pyridin-6-yl)-2-methyl-4-(3-(methylamino)pyrrolidin-1-yl)-2H-indazole-7-carboxamide (5.2 mg, 23%) as a solid. LCMS (ES, m/z): 422 [M+H]^{+ 1}H NMR (400 MHz, Methanol-d₄) δ 8.99 (d, J=1.6 Hz, 1H), 8.51 (s, 1H), 8.03 (d, J=8.4 Hz, 1H), 7.69 (d, J=2.9 Hz, 1H), 7.11 (dd, J=11.9, 1.6 Hz, 1H), 6.07 (d, J=8.4 Hz, 1H), 4.29 (s, 3H), 3.94-3.76 (m, 2H), 3.72-3.66 (m, 1H), 3.54-3.53 (m, 2H), 2.54 (s, 3H), 2.44 (s, 3H), 2.40-2.34 (m, 1H), 2.10-2.02 (m, 1H).

### Example 142: Synthesis of Compound 391

### Synthesis of Intermediate C179

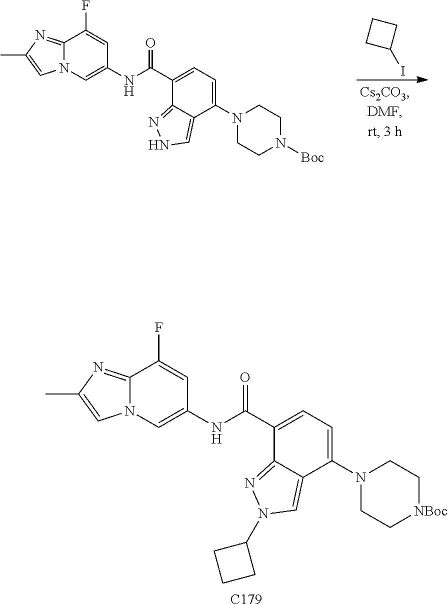 [SMILES: Cc1cn2cc(NC(=O)c3ccc(N4CCN(C(=O)OC(C)(C)C)CC4)c4c[nH]nc34)cc(F)c2n1 ; IC1CCC1 ; Cc1cn2cc(NC(=O)c3ccc(N4CCN(C(=O)OC(C)(C)C)CC4)c4cn(C5CCC5)nc34)cc(F)c2n1 ; "C179"]

To a stirred mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.20 mmol, 1.0 equiv) and iodocyclobutane (55.3 mg, 0.30 mmol, 1.5 equiv) in DMF (2 mL) was added Cs₂CO₃ (198.0 mg, 0.60 mmol, 3.0 equiv) at room temperature. The resulting mixture was stirred for 3 h at room temperature. The resulting mixture was diluted with water. The resulting mixture was extracted with ethyl acetate (3×15 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:4) to afford tert-butyl 4-[2-cyclobutyl-7-({8-fluoro-2-methylimidazo[1,2-a]pyridin-6-yl}carbamoyl) indazol-4-yl] piperazine-1-carboxylate (C179, 59 mg, 53%) as a solid. LCMS (ES, m/z): 548 [M+H]⁺

### Synthesis of Compound 391

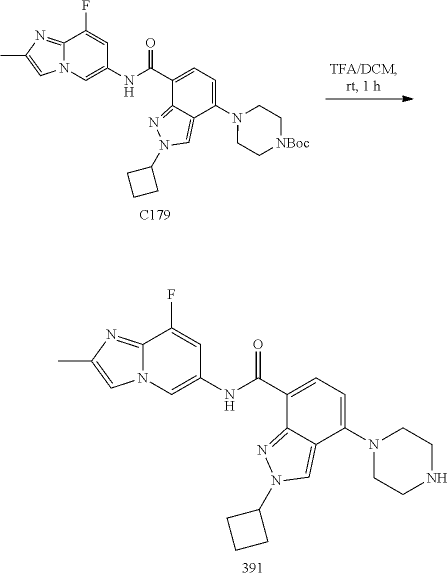

A solution of tert-butyl 4-[2-cyclobutyl-7-({8-fluoro-2-methylimidazo[1,2-a]pyridin-6-yl}carbamoyl) indazol-4-yl]piperazine-1-carboxylate (59.0 mg, 0.10 mmol, 1.0 equiv) in DCM (0.6 mL) was treated with TFA (0.6 mL) at room temperature. The resulting mixture was stirred for 1 h at room temperature. The resulting mixture was concentrated under reduced pressure. The crude product was purified by Prep-HPLC (Condition 10, Gradient 3) to afford 2-cyclobutyl-N-{8-fluoro-2-methylimidazo[1,2-a]pyridin-6-yl}-4-(piperazin-1-yl) indazole-7-carboxamide (Compound 391, 9.7 mg, 20%) as a solid. LCMS (ES, m/z): 448 [M+H]^{+ 1}H NMR (400 MHz, DMSO-d₆) δ 11.12 (s, 1H), 9.22 (s, 1H), 8.89 (s, 1H), 7.99 (d, J=7.8 Hz, 1H), 7.92 (s, 1H), 7.24 (d, J=12.3 Hz, 1H), 6.50 (d, J=8.1 Hz, 1H), 5.35-5.28 (m, 1H), 3.37-3.34 (m, 5H), 2.95-2.93 (m, 4H), 2.82-2.71 (m, 2H), 2.60-2.59 (m, 2H), 2.36 (s, 3H), 1.96 (q, J=11.5, 11.1 Hz, 2H).

### Example 143: Synthesis of Compound 392

### Synthesis of Intermediate C180

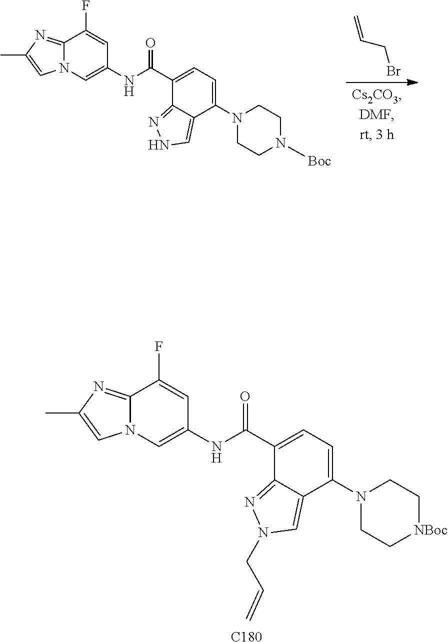

To a stirred mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.20 mmol, 1.0 equiv) and allyl bromide (36.7 mg, 0.30 mmol, 1.5 equiv) in DMF (2 mL) was added Cs₂CO₃ (198.0 mg, 0.61 mmol, 3.0 equiv) at room temperature. The resulting mixture was stirred for 3 h at room temperature. The resulting mixture was diluted with water. The resulting mixture was extracted with ethyl acetate (2×10 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:4) to afford tert-butyl 4-[7-({8-fluoro-2-methylimidazo[1,2-a]pyridin-6-yl}carbamoyl)-2-(prop-2-en-1-yl) indazol-4-yl] piperazine-1-carboxylate (C180, 52 mg, 48%) as a solid. LCMS (ES, m/z): 534 [M+H]⁺

### Synthesis of Compound 392

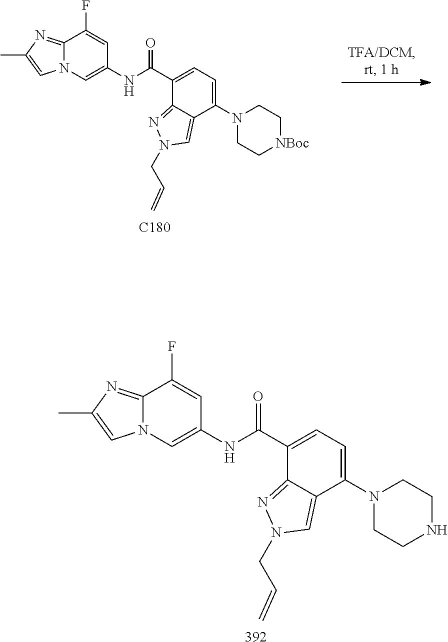

A solution of tert-butyl 4-[7-({8-fluoro-2-methylimidazo[1,2-a]pyridin-6-yl}carbamoyl)-2-(prop-2-en-1-yl) indazol-4-yl]piperazine-1-carboxylate (52 mg, 0.10 mmol, 1 equiv) in DCM (0.5 mL) was treated with TFA (0.5 mL) at room temperature. The resulting mixture was stirred for 1 hr at room temperature. The resulting mixture was concentrated under reduced pressure. The crude product was purified by Prep-HPLC (Condition 10, Gradient 3) to afford N-{8-fluoro-2-methylimidazo[1,2-a]pyridin-6-yl}-4-(piperazin-1-yl)-2-(prop-2-en-1-yl) indazole-7-carboxamide (Compound 392, 12.7 mg, 30%) as a solid. LCMS (ES, m/z): 434 [M+H]^{+ 1}H NMR (400 MHz, DMSO-d₆) δ 11.13 (s, 1H), 9.22 (d, J=1.6 Hz, 1H), 8.82 (s, 1H), 7.99 (d, J=8.1 Hz, 1H), 7.91 (d, J=3.1 Hz, 1H), 7.24 (dd, J=12.3, 1.7 Hz, 1H), 6.50 (d, J=8.1 Hz, 1H), 6.26 (ddd, J=16.4, 10.3, 6.0 Hz, 1H), 5.45-5.29 (m, 2H), 5.22 (d, J=6.1 Hz, 2H), 3.36 (t, J=5.0 Hz, 4H), 2.92 (t, J=4.9 Hz, 4H), 2.35 (s, 3H).

### Example 144: Synthesis of Compound 394

### Synthesis of Intermediate C181

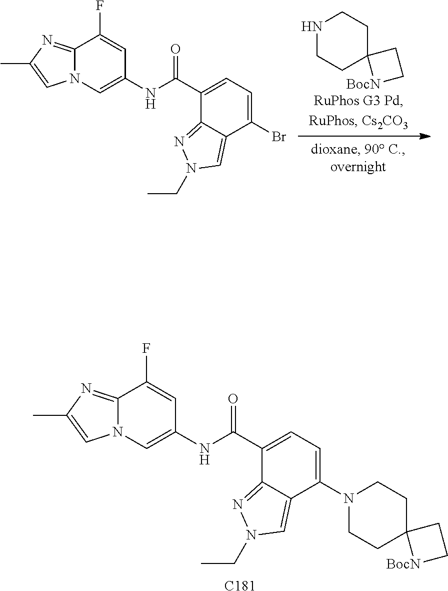 [SMILES: CCn1cc2c(Br)ccc(C(=O)Nc3cc(F)c4nc(C)cn4c3)c2n1 ; CC(C)(C)OC(=O)N1CCC12CCNCC2 ; CCn1cc2c(N3CCC4(CC3)CCN4C(=O)OC(C)(C)C)ccc(C(=O)Nc3cc(F)c4nc(C)cn4c3)c2n1 ; "C181"]

To a stirred solution of 4-bromo-2-ethyl-N-{8-fluoro-2-methylimidazo[1,2-a]pyridin-6-yl}indazole-7-carboxamide (90 mg, 0.22 mmol, 1 equiv) and tert-butyl 1,7-diazaspiro[3.5]nonane-1-carboxylate (49 mg, 0.22 mmol, 1 equiv) in dioxane (10 mL) were added Cs₂CO₃ (211 mg, 0.66 mmol, 3 equiv), RuPhos (20 mg, 0.044 mmol, 0.2 equiv) and RuPhos Palladacycle Gen.3 (18 mg, 0.022 mmol, 0.1 equiv) in portions at room temperature under nitrogen atmosphere. The resulting mixture was stirred overnight at 90° C. under nitrogen atmosphere. The mixture was allowed to cool down to room temperature. The resulting mixture was concentrated under reduced pressure. The residue was dissolved in water (20 mL). The resulting mixture was extracted with ethyl acetate (3×5 mL). The combined organic layers were washed with brine (1×10 mL), dried over anhydrous Na₂SO₄. After filtration, the filtrate was concentrated under reduced pressure. The residue was purified by reverse flash chromatography (Condition 5, Gradient 1) to afford tert-butyl 7-[2-ethyl-7-({8-fluoro-2-methylimidazo[1,2-a]pyridin-6-yl}carbamoyl) indazol-4-yl]-1,7-diazaspiro[3.5]nonane-1-carboxylate (75 mg, 61%) as a solid. LCMS (ES, m/z): 562 [M+H]⁺

### Synthesis of Compound 394

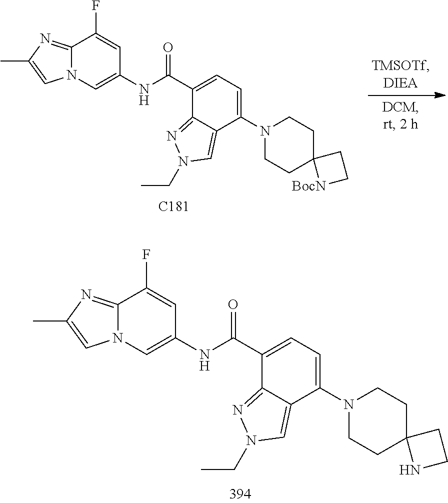 [SMILES: CCn1cc2c(N3CCC4(CC3)CCN4C(=O)OC(C)(C)C)ccc(C(=O)Nc3cc(F)c4nc(C)cn4c3)c2n1 ; "C181" ; CCn1cc2c(N3CCC4(CCN4)CC3)ccc(C(=O)Nc3cc(F)c4nc(C)cn4c3)c2n1]

To a stirred solution of tert-butyl 7-[2-ethyl-7-({8-fluoro-2-methylimidazo[1,2-a]pyridin-6-yl}carbamoyl) indazol-4-yl]-1,7-diazaspiro[3.5]nonane-1-carboxylate (90 mg, 0.16 mmol, 1 equiv) and DIEA (31 mg, 0.24 mmol, 1.5 equiv) in DCM (5 mL) was added Trimethylsilyl trifluorornethanesulfonate (107 mg, 0.48 mmol, 3 equiv) dropwise at 0° C. under nitrogen atmosphere. The resulting mixture was stirred for 2 hr at room temperature. The reaction was quenched with water (2 mL) at room temperature. The resulting mixture was concentrated under vacuum. The residue was purified by prep-HPLC (Condition 12, Gradient 1) to afford 4-{1,7-diazaspiro[3.5]nonan-7-yl}-2-ethyl-N-{8-fluoro-2-methylimidazo[1,2-a]pyridin-6-yl}indazole-7-carboxamide (Compound 394, 30 mg, 40%) as a solid. LCMS (ES, m/z): 462 [M+H]^{+ 1}H NMR (300 MHz, DMSO-d₆) δ 11.10 (s, 1H), 9.21 (d, J=1.6 Hz, 1H), 8.80 (s, 1H), 7.97 (d, J=8.1 Hz, 1H), 7.91 (d, J=3.1 Hz, 1H), 7.30 (dd, J=12.4, 1.7 Hz, 1H), 6.50 (d, J=8.2 Hz, 1H), 4.60 (q, J=7.2 Hz, 2H), 3.99-3.70 (m, 1H), 3.60-3.40 (m, 3H), 3.29 (s, 2H), 2.35 (s, 3H), 2.07 (t, J=7.5 Hz, 2H), 1.96-1.77 (m, 4H), 1.62 (t, J=7.2 Hz, 3H).

### Example 145: Synthesis of Compound 395

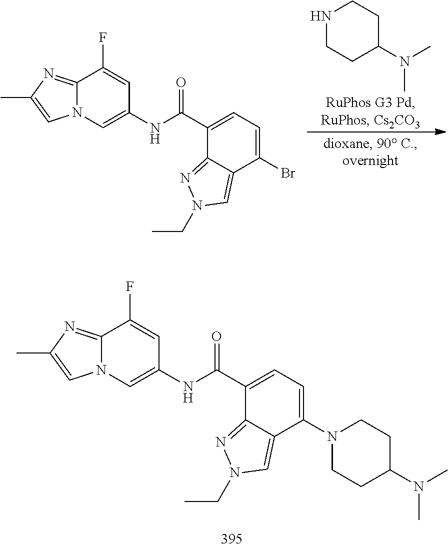

To a stirred solution of 4-bromo-2-ethyl-N-{8-fluoro-2-methylimidazo[1,2-a]pyridin-6-yl}indazole-7-carboxamide (90 mg, 0.22 mmol, 1 equiv) and N,N-dimethylpiperidin-4-amine (28 mg, 0.22 mmol, 1 equiv) in dioxane (10 mL) were added Cs₂CO₃ (211 mg, 0.66 mmol, 3 equiv), RuPhos (20 mg, 0.044 mmol, 0.2 equiv) and RuPhos Palladacycle Gen.3 (18 mg, 0.022 mmol, 0.1 equiv) in portions at room temperature under nitrogen atmosphere. The resulting mixture was stirred for overnight at 90° C. under nitrogen atmosphere. The mixture was allowed to cool down to room temperature. The resulting mixture was concentrated under reduced pressure. The residue was dissolved in water (20 mL). The resulting mixture was extracted with ethyl acetate (3×5 mL). The combined organic layers were washed with brine (1×10 mL), dried over anhydrous Na₂SO₄. After filtration, the filtrate was concentrated under reduced pressure. The residue was purified by prep-HPLC (Condition 12, Gradient 1) to afford 4-[4-(dimethylamino)piperidin-1-yl]-2-ethyl-N-{8-fluoro-2-methylimidazo[1,2-a]pyridin-6-yl}indazole-7-carboxamide (Compound 395, 30 mg, 29%) as a solid. LCMS (ES, m/z): 464 [M+H]^{+ 1}H NMR (300 MHz, DMSO-d₆) δ 11.10 (s, 1H), 9.21 (d, J=1.6 Hz, 1H), 8.81 (s, 1H), 7.97 (d, J=8.1 Hz, 1H), 7.90 (d, J=3.1 Hz, 1H), 7.30 (dd, J=12.4, 1.7 Hz, 1H), 6.49 (d, J=8.2 Hz, 1H), 4.60 (q, J=7.3 Hz, 2H), 3.96 (d, J=12.5 Hz, 2H), 2.97 (t, J=11.8 Hz, 2H), 2.35 (s, 4H), 2.22 (s, 6H), 1.90 (d, J=12.2 Hz, 2H), 1.62 (t, J=7.2 Hz, 4H).

### Example 146: Synthesis of Compound 396

### Synthesis of Intermediate C182

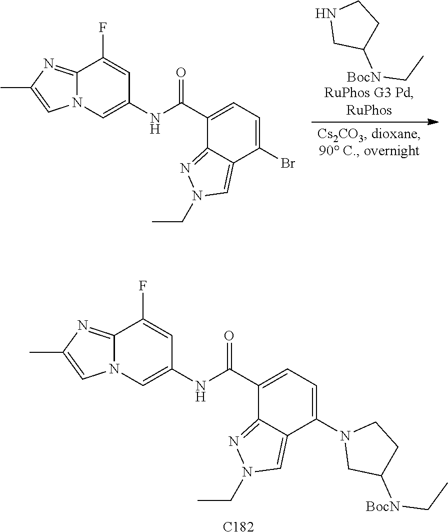 [SMILES: CCn1cc2c(Br)ccc(C(=O)Nc3cc(F)c4nc(C)cn4c3)c2n1 ; CCN(C(=O)OC(C)(C)C)C1CCNC1 ; CCN(C(=O)OC(C)(C)C)C1CCN(c2ccc(C(=O)Nc3cc(F)c4nc(C)cn4c3)c3nn(CC)cc23)C1 ; "C182"]

To a stirred solution of 4-bromo-2-ethyl-N-{8-fluoro-2-methylimidazo[1,2-a]pyridin-6-yl}indazole-7-carboxamide (180 mg, 0.43 mmol, 1 equiv) and tert-butyl N-ethyl-N-(pyrrolidin-3-yl)carbamate (93 mg, 0.43 mmol, 1 equiv) in dioxane (10 mL) were added Cs₂CO₃ (423 mg, 1.29 mmol, 3 equiv), RuPhos (40 mg, 0.086 mmol, 0.2 equiv) and RuPhos Palladacycle Gen.3 (36 mg, 0.043 mmol, 0.1 equiv) in portions at room temperature under nitrogen atmosphere. The resulting mixture was stirred for overnight at 90° C. under nitrogen atmosphere. The mixture was allowed to cool down to room temperature. The resulting mixture was concentrated under reduced pressure. The residue was dissolved in water (20 mL). The resulting mixture was extracted with ethyl acetate (3×5 mL). The combined organic layers were washed with brine (1×10 mL), dried over anhydrous Na₂SO₄. After filtration, the filtrate was concentrated under reduced pressure. The residue was purified by reverse flash chromatography (Condition 5, Gradient 2) to afford tert-butyl N-ethyl-N-{1-[2-ethyl-7-({8-fluoro-2-methylimidazo[1,2-a]pyridin-6-yl}carbamoyl) indazol-4-yl]pyrrolidin-3-yl}carbamate (C182, 100 mg, 42%) as a solid. LCMS (ES, m/z): 550 [M+H]⁺

### Synthesis of Compound 396

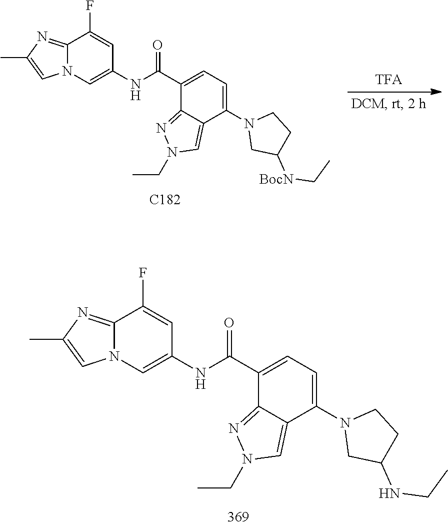

A solution of tert-butyl N-ethyl-N-{1-[2-ethyl-7-({8-fluoro-2-methylimidazo[1,2-a]pyridin-6-yl}carbamoyl) indazol-4-yl]pyrrolidin-3-yl}carbamate (140 mg, 0.255 mmol, 1 equiv) in trifluoroacetic acid (3 mL) and DCM (3 mL) was stirred for 2 h at room temperature. The resulting mixture was concentrated under reduced pressure. The residue was basified to pH 8 with 7 M NH₃(g) in MeOH. The resulting mixture was concentrated under vacuum. The residue was purified by prep-HPLC (Condition 12, Gradient 1) to afford 2-ethyl-4-[3-(ethylamino)pyrrolidin-1-yl]-N-{8-fluoro-2-methylimidazo[1,2-a]pyridin-6-yl}indazole-7-carboxamide (Compound 396, 60 mg, 52%) as a solid. LCMS (ES, m/z): 450 [M+H]^{+ 1}H NMR (300 MHz, DMSO-d₆) δ 11.05 (s, 1H), 9.19 (d, J=1.6 Hz, 1H), 8.84 (s, 1H), 7.93 (d, J=8.3 Hz, 1H), 7.89 (d, J=3.1 Hz, 1H), 7.26 (dd, J=12.4, 1.7 Hz, 1H), 6.01 (d, J=8.4 Hz, 1H), 4.57 (q, J=7.3 Hz, 2H), 3.77 (dd, J=13.6, 7.0 Hz, 2H), 3.70-3.57 (m, 1H), 3.53-3.37 (m, 2H), 2.63 (q, J=7.1 Hz, 2H), 2.35 (s, 3H), 2.16 (dd, J=12.3, 6.0 Hz, 1H), 1.90 (dd, J=12.1, 6.3 Hz, 1H), 1.61 (t, J=7.2 Hz, 3H), 1.05 (t, J=7.1 Hz, 3H).

### Example 147: Synthesis of Compound 397

### Synthesis of Intermediate C183

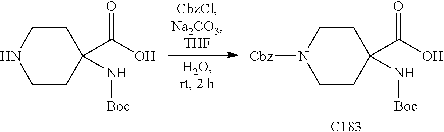 [SMILES: CC(C)(C)OC(=O)NC1(C(=O)O)CCNCC1 ; CC(C)(C)OC(=O)NC1(C(=O)O)CCN(C(=O)OCc2ccccc2)CC1 ; "C183"]

To a stirred solution of 4-[(tert-butoxycarbonyl)amino]piperidine-4-carboxylic acid (1.5 g, 6.140 mmol, 1.0 equiv) and sodium methaneperoxoate sodium (1.3 g, 12.280 mmol, 2.0 equiv) in tetrahydrofuran (15 mL), water (15 mL) was added benzyl chloroformate (1.2 g, 7.368 mmol, 1.2 equiv) dropwise at 0° C. under nitrogen atmosphere. The resulting mixture was stirred for 2 hr at room temperature. The resulting mixture was diluted with water (30 mL). The mixture was acidified to PH 5 with HCl (aq.). The resulting mixture was extracted with ethyl acetate (2×50 mL). The combined organic layers were washed with brine (1×30 mL), dried over anhydrous Na₂SO₄. After filtration, the filtrate was concentrated under reduced pressure. The residue was purified by silica gel column chromatography, eluted with CH₂Cl₂/MeOH (10:1) to afford 1-[(benzyloxy)carbonyl]-4-[(tert-butoxycarbonyl)amino]piperidine-4-carboxylic acid (C183, 1.4 g, 55%) as an oil. LCMS (ES, m/z): 379 [M+H]⁺

### Synthesis of Intermediate C184

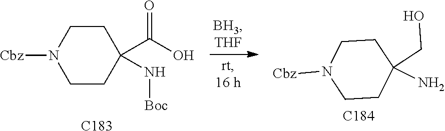 [SMILES: CC(C)(C)OC(=O)NC1(C(=O)O)CCN(C(=O)OCc2ccccc2)CC1 ; NC1(CO)CCN(C(=O)OCc2ccccc2)CC1 ; "C184" ; "C183"]

To a stirred solution of 1-[(benzyloxy)carbonyl]-4-[(tert-butoxycarbonyl)amino]piperidine-4-carboxylic acid (1.4 g, 3.700 mmol, 1.0 equiv) in tetrahydrofuran (15 mL) was added borane-tetrahydrofuran complex, 1M (40 mL) dropwise at room temperature under nitrogen atmosphere. The resulting mixture was stirred for 16 hr at 0° C. under nitrogen atmosphere. The reaction was quenched with methanol at 0° C. The resulting mixture was concentrated under reduced pressure. The residue was purified by silica gel column chromatography, eluted with CH₂Cl₂/MeOH (10:1) to afford benzyl 4-amino-4-(hydroxymethyl)piperidine-1-carboxylate (C184, 0.78 g, 71%) as an oil. LCMS (ES, m/z): 265 [M+H]⁺

### Synthesis of Intermediate C185

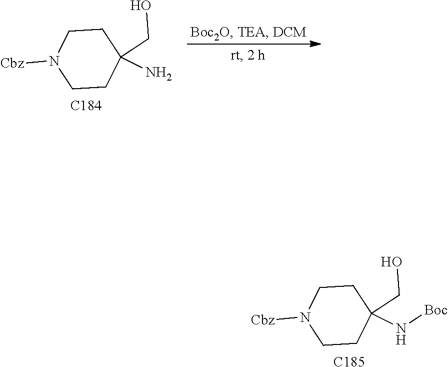 [SMILES: NC1(CO)CCN(C(=O)OCc2ccccc2)CC1 ; "C184" ; CC(C)(C)OC(=O)NC1(CO)CCN(C(=O)OCc2ccccc2)CC1 ; "C185"]

To a stirred mixture of benzyl 4-amino-4-(hydroxymethyl)piperidine-1-carboxylate (0.7 g, 2.762 mmol, 1.0 equiv) and triethylamine (0.8 g, 8.286 mmol, 3.0 equiv) in DCM (10 mL) was added di-tert-butyl dicarbonate (0.7 g, 3.314 mmol, 1.2 equiv) in portions at 0° C. under nitrogen atmosphere. The resulting mixture was stirred for 2 hr at room temperature. The resulting mixture was concentrated under reduced pressure. The residue was purified by silica gel column chromatography, eluted with PE/EA (1:1) to afford benzyl 4-[(tert-butoxycarbonyl) amino]-4-(hydroxymethyl) piperidine-1-carboxylate (C185, 0.8 g, 71%) as an oil. LCMS (ES, m/z): 365 [M+H]⁺

### Synthesis of Intermediate C186

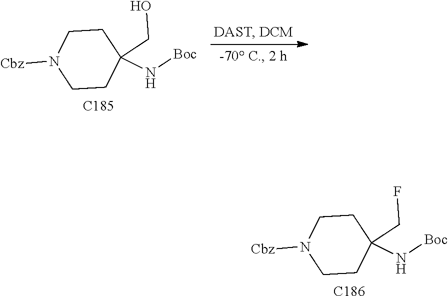 [SMILES: CC(C)(C)OC(=O)NC1(CO)CCN(C(=O)OCc2ccccc2)CC1 ; "C185" ; CC(C)(C)OC(=O)NC1(CF)CCN(C(=O)OCc2ccccc2)CC1 ; "C186"]

To a stirred solution of benzyl 4-[(tert-butoxycarbonyl)amino]-4-(hydroxymethyl)piperidine-1-carboxylate (700.0 mg, 1.921 mmol, 1.0 equiv) in DCM (8 mL) was added Diethylaminosulfur trifluoride (464.4 mg, 2.881 mmol, 1.5 equiv) dropwise at 0° C. under nitrogen atmosphere. The reaction was quenched with sat. NaHCO₃ (aq.) at 0° C. The resulting mixture was extracted with ethyl acetate (2×20 mL). The combined organic layers were washed with brine (1×20 mL), dried over anhydrous Na₂SO₄. After filtration, the filtrate was concentrated under reduced pressure.

LCMS (ES, m/z): 367 [M+H]⁺

### Synthesis of Intermediate C187

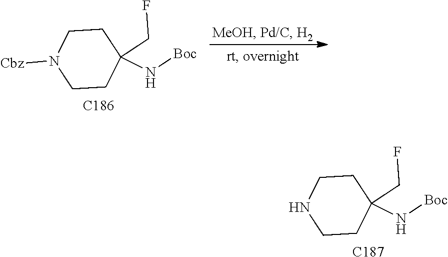 [SMILES: CC(C)(C)OC(=O)NC1(CF)CCN(C(=O)OCc2ccccc2)CC1 ; "C186" ; CC(C)(C)OC(=O)NC1(CF)CCNCC1 ; "C187"]

To a solution of benzyl 4-[(tert-butoxycarbonyl)amino]-4-(fluoromethyl)piperidine-1-carboxylate (290.0 mg, 0.791 mmol, 1.0 equiv) in 10 mL methanol was added Pd/C (10%, 250 mg) under nitrogen atmosphere in a mL round-bottom flask. The mixture was hydrogenated at room temperature for overnight under hydrogen atmosphere using a hydrogen balloon. The mixture was filtered through a Celite pad and concentrated under reduced pressure to yield tert-butyl (4-(fluoromethyl)piperidin-4-yl)carbamate (C187, 130 mg, 65%) as a solid. LCMS (ES, m/z): 233 [M+H]⁺

### Synthesis of Intermediate C188

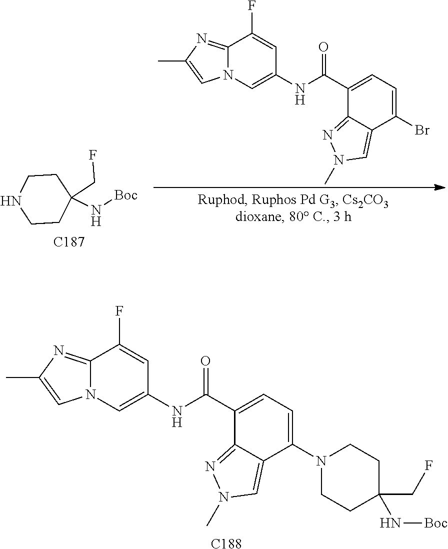 [SMILES: CC(C)(C)OC(=O)NC1(CF)CCNCC1 ; Cc1cn2cc(NC(=O)c3ccc(Br)c4cn(C)nc34)cc(F)c2n1 ; "C187" ; Cc1cn2cc(NC(=O)c3ccc(N4CCC(CF)(NC(=O)OC(C)(C)C)CC4)c4cn(C)nc34)cc(F)c2n1 ; "C188"]

To a solution of tert-butyl N-[4-(fluoromethyl) piperidin-4-yl]carbamate (80.0 mg, 0.344 mmol, 1 equiv) and 4-bromo-N-{8-fluoro-2-methylimidazo[1,2-a]pyridin-6-yl}-2-methylindazole-7-carboxamide (138.5 mg, 0.344 mmol, 1.0 equiv) in dioxane (4 mL) were added Cs₂CO₃ (280.5 mg, 0.860 mmol, 2.5 equiv) and Ruphos (32.1 mg, 0.069 mmol, 0.2 equiv), RuPhos Palladacycle Gen.3 (28.8 mg, 0.034 mmol, 0.1 equiv). After stirring for 3 hr at 80° C. under a nitrogen atmosphere, the resulting mixture was concentrated under reduced pressure. The residue was purified by silica gel column chromatography, eluted with CH₂Cl₂/MeOH (30:1) to afford tert-butyl N-{1-[7-({8-fluoro-2-methylimidazo[1,2-a]pyridin-6-yl}carbamoyl)-2-methylindazol-4-yl]-4-(fluoromethyl)piperidin-4-yl}carbamate (C188, 120 mg, 53.50%) as a solid. LCMS (ES, m/z): 554 [M+H]⁺

### Synthesis of Intermediate Compound 397

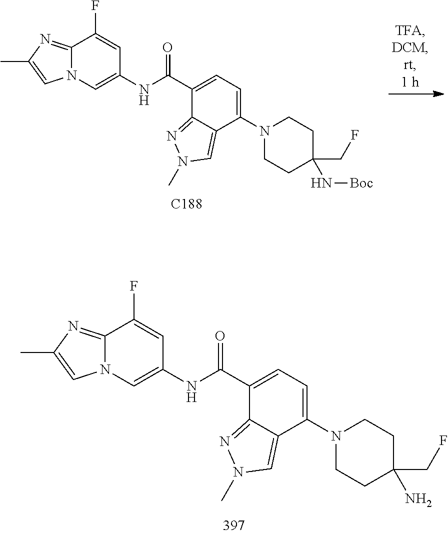 [SMILES: Cc1cn2cc(NC(=O)c3ccc(N4CCC(CF)(NC(=O)OC(C)(C)C)CC4)c4cn(C)nc34)cc(F)c2n1 ; "C188" ; Cc1cn2cc(NC(=O)c3ccc(N4CCC(N)(CF)CC4)c4cn(C)nc34)cc(F)c2n1]

To a stirred solution of tert-butyl N-{1-[7-({8-fluoro-2-methylimidazo[1,2-a]pyridin-6-yl}carbamoyl)-2-methylindazol-4-yl]-4-(fluoromethyl)piperidin-4-yl}carbamate (80.0 mg, 0.145 mmol, 1.0 equiv) in DCM (1 mL) was added trifluoroacetaldehyde (0.5 mL) dropwise at 0° C. The resulting mixture was stirred for 1 hr at room temperature. The resulting mixture was concentrated under reduced pressure. The crude product was purified by Prep-HPLC (Condition 13, Gradient 1) to afford 4-[4-amino-4-(fluoromethyl)piperidin-1-yl]-N-{8-fluoro-2-methylimidazo[1,2-a]pyridin-6-yl}-2-methylindazole-7-carboxamide (C187, 9 mg, 13%) as a light yellow solid. LCMS (ES, m/z): 454 [M+H]^{+ 1}H NMR (300 MHz, DMSO-d₆) δ 11.07 (s, 1H), 9.22 (d, J=1.6 Hz, 1H), 8.82 (d, J=5.8 Hz, 1H), 7.98 (d, J=8.1 Hz, 1H), 7.91 (d, J=3.2 Hz, 1H), 7.35 (dd, J=12.3, 1.7 Hz, 1H), 6.55 (d, J=8.2 Hz, 1H), 4.30 (s, 3H), 3.81 (d, J=12.7 Hz, 2H), 3.25-3.24 (m, 4H), 2.76 (d, J=19.4 Hz, 2H), 2.35 (s, 3H), 1.95-1.83 (m, 4H).

### Example 148: Synthesis of Compound 398

### Synthesis of Intermediate C189

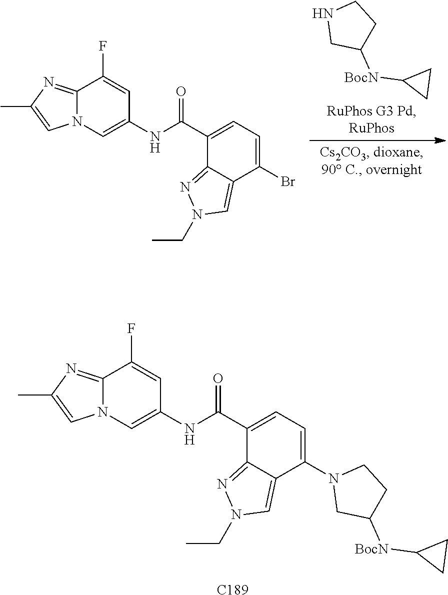 [SMILES: CCn1cc2c(Br)ccc(C(=O)Nc3cc(F)c4nc(C)cn4c3)c2n1 ; CC(C)(C)OC(=O)N(C1CC1)C1CCNC1 ; CCn1cc2c(N3CCC(N(C(=O)OC(C)(C)C)C4CC4)C3)ccc(C(=O)Nc3cc(F)c4nc(C)cn4c3)c2n1 ; "C189"]

To a stirred solution of 4-bromo-2-ethyl-N-{8-fluoro-2-methylimidazo[1,2-a]pyridin-6-yl}indazole-7-carboxamide (190 mg, 0.456 mmol, 1 equiv) and tert-butyl N-cyclopropyl-N-(pyrrolidin-3-yl)carbamate (103 mg, 0.456 mmol, 1 equiv) in dioxane (10 mL) were added Cs₂CO₃ (446 mg, 1.368 mmol, 3 equiv), RuPhos (43 mg, 0.091 mmol, 0.2 equiv) and RuPhos Palladacycle Gen.3 (21 mg, 0.046 mmol, 0.1 equiv) in portions at room temperature under nitrogen atmosphere. The resulting mixture was stirred for overnight at 90° C. under nitrogen atmosphere. The mixture was allowed to cool down to room temperature. The resulting mixture was concentrated under reduced pressure. The residue was purified by reverse flash chromatography (Condition 5, Gradient 2) to afford tert-butyl N-cyclopropyl-N-{1-[2-ethyl-7-({8-fluoro-2-methylimidazo[1,2-a]pyridin-6-yl}carbamoyl) indazol-4-yl]pyrrolidin-3-yl}carbamate (C189, 200 mg, 78%) as a solid. LCMS (ES, m/z): 562 [M+H]⁺

### Synthesis of Compound 398

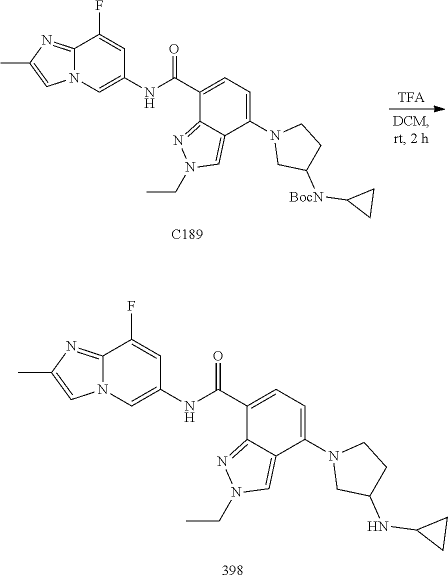 [SMILES: CCn1cc2c(N3CCC(N(C(=O)OC(C)(C)C)C4CC4)C3)ccc(C(=O)Nc3cc(F)c4nc(C)cn4c3)c2n1 ; "C189" ; CCn1cc2c(N3CCC(NC4CC4)C3)ccc(C(=O)Nc3cc(F)c4nc(C)cn4c3)c2n1]

A solution of tert-butyl N-cyclopropyl-N-{1-[2-ethyl-7-({8-fluoro-2-methylimidazo[1,2-a]pyridin-6-yl}carbamoyl) indazol-4-yl]pyrrolidin-3-yl}carbamate (90 mg, 0.160 mmol, 1 equiv) in trifluoroacetic acid (2 mL) and DCM (2 mL) was stirred for 2 h at room temperature. The resulting mixture was concentrated under reduced pressure. The residue was basified to pH 8 with 7 M NH₃(g) in methanol. The resulting mixture was concentrated under reduced pressure. The residue was purified by prep-HPLC (Condition 10, Gradient 4) to afford 4-[3-(cyclopropylamino)pyrrolidin-1-yl]-2-ethyl-N-{8-fluoro-2-methylimidazo[1,2-a]pyridin-6-yl}indazole-7-carboxamide (Compound 398, 60 mg, 81%) as a solid. LCMS (ES, m/z): 462 [M+H]^{+ 1}H NMR (300 MHz, DMSO-d₆) δ 11.04 (s, 1H), 9.19 (d, J=1.6 Hz, 1H), 8.83 (s, 1H), 7.97-7.85 (m, 2H), 7.25 (dd, J=12.4, 1.7 Hz, 1H), 6.00 (d, J=8.4 Hz, 1H), 4.57 (q, J=7.3 Hz, 2H), 3.77 (dd, J=12.8, 6.6 Hz, 2H), 3.69-3.41 (m, 3H), 2.71 (m, 1H) 2.35 (s, 3H), 2.17 (td, J=7.1, 6.5, 3.9 Hz, 2H), 1.98 (dd, J=12.3, 6.2 Hz, 1H), 1.61 (t, J=7.2 Hz, 3H), 0.42 (d, J=6.6 Hz, 2H), 0.25 (dq, J=9.7, 6.2, 4.7 Hz, 2H).

### Example 149: Synthesis of Compounds 399 and 400

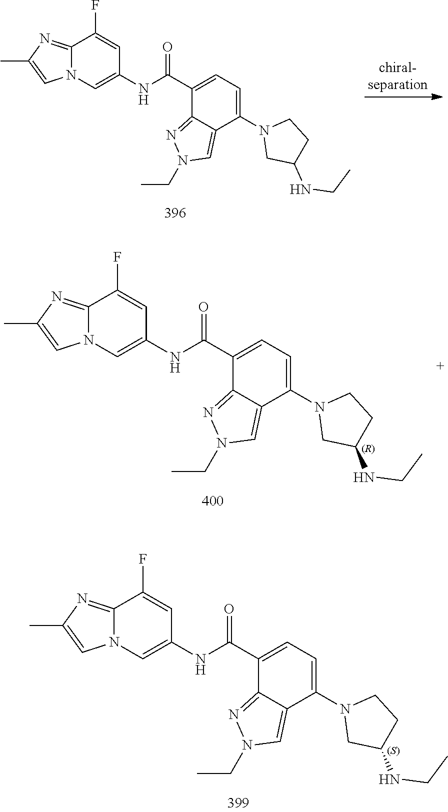

Compound 396 was separated by Prep-Chiral-HPLC (Condition 3, Gradient 1) to afford (R)-2-ethyl-4-(3-(ethylamino)pyrrolidin-1-yl)-N-(8-fluoro-2-methylimidazo[1,2-a]pyridin-6-yl)-2H-indazole-7-carboxamide (Compound 400, 18.4 mg, 30%) as a white solid and (S)-2-ethyl-4-(3-(ethylamino)pyrrolidin-1-yl)-N-(8-fluoro-2-methylimidazo[1,2-a]pyridin-6-yl)-2H-indazole-7-carboxamide (Compound 399, 18.4 mg, 30%) as a solid.

| Compound No. | Analysis Data |
|---|---|
| 399 | LCMS (ES, m/z): 450 [M + H]⁺ |
| | ¹H NMR (300 MHz, DMSO-d₆) δ 11.05 (s, 1H), 9.19 (d, J = 1.6 Hz, 1H), |
| | 8.84 (s, 1H), 7.93 (d, J = 8.3 Hz, 1H), 7.89 (d, J = 3.1 Hz, 1H), 7.26 (dd, J = |
| | 12.4, 1.7 Hz, 1H), 6.01 (d, J = 8.4 Hz, 1H), 4.57 (q, J = 7.3 Hz, 2H), 3.77 (dd, |
| | J = 13.6, 7.0 Hz, 2H), 3.70-3.57 (m, 1H), 3.53-3.37 (m, 2H), 2.63 (q, J = 7.1 |
| | Hz, 2H), 2.35 (s, 3H), 2.16 (dd, J = 12.3, 6.0 Hz, 1H), 1.90 (dd, J = 12.1, 6.3 |
| | Hz, 1H), 1.61 (t, J = 7.2 Hz, 3H), 1.05 (t, J = 7.1 Hz, 3H). |
| 400 | LCMS (ES, m/z): 450 [M + H]⁺ |
| | ¹H NMR (300 MHz, DMSO-d₆) δ 11.05 (s, 1H), 9.19 (d, J = 1.6 Hz, 1H), |
| | 8.84 (s, 1H), 7.93 (d, J = 8.3 Hz, 1H), 7.89 (d, J = 3.1 Hz, 1H), 7.26 (dd, J = |
| | 12.4, 1.7 Hz, 1H), 6.01 (d, J = 8.4 Hz, 1H), 4.57 (q, J = 7.3 Hz, 2H), 3.77 (dd, |
| | J = 13.6, 7.0 Hz, 2H), 3.70-3.57 (m, 1H), 3.53-3.37 (m, 2H), 2.63 (q, J = 7.1 |
| | Hz, 2H), 2.35 (s, 3H), 2.16 (dd, J = 12.3, 6.0 Hz, 1H), 1.90 (dd, J = 12.1, 6.3 |
| | Hz, 1H), 1.61 (t, J = 7.2 Hz, 3H), 1.05 (t, J = 7.1 Hz, 3H). |

### Example 150: Synthesis of Compound 401

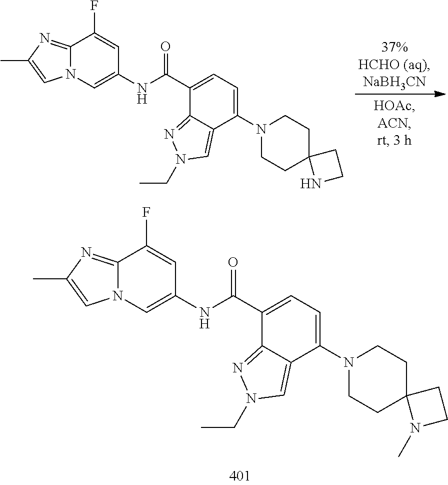

To a stirred solution of 4-{1,7-diazaspiro[3.5]nonan-7-yl}-2-ethyl-N-{8-fluoro-2-methylimidazo[1,2-a]pyridin-6-yl}indazole-7-carboxamide (280 mg, 0.607 mmol, 1 equiv) and 37% HCHO (91 mg, 3.035 mmol, 5 equiv) in ACN (5 mL) was added NaBH₃CN (114.3 mg, 1.821 mmol, 3 equiv) in portions at room temperature. The resulting mixture was stirred for 30 min at room temperature. To the above mixture was added HOAc (364.3 mg, 6.070 mmol, 10 equiv) dropwise at room temperature. The resulting mixture was stirred for additional 2 hr at room temperature. The reaction was quenched with water (2 mL) at room temperature. The resulting mixture was concentrated under vacuum. The residue was purified by reverse flash chromatography (Condition 3, Gradient 4) to afford 2-ethyl-N-(8-fluoro-2-methylimidazo[1,2-a]pyridin-6-yl)-4-(1-methyl-1,7-diazaspiro[3.5]nonan-7-yl)-2H-indazole-7-carboxamide 2,2,2-trifluoroacetate (Compound 401, 100 mg, 27%) as a solid. LCMS (ES, m/z): 476 [M+H]^{+ 1}H NMR (300 MHz, Methanol-d₄) δ 9.52 (d, J=1.6 Hz, 1H), 8.64 (s, 1H), 8.20-8.07 (m, 2H), 7.99 (dd, J=11.3, 1.6 Hz, 1H), 6.64 (d, J=8.1 Hz, 1H), 4.67 (q, J=7.3 Hz, 2H), 4.33-4.21 (m, 1H), 4.16-4.01 (m, 2H), 3.96 (d, J=10.3 Hz, 1H), 3.16 (q, J=12.5 Hz, 2H), 2.87 (s, 3H), 2.68-2.56 (m, 5H), 2.44 (d, J=12.2 Hz, 1H), 2.37-2.23 (m, 3H), 1.73 (t, J=7.3 Hz, 3H).

### Example 151: Synthesis of Compound 402

### Synthesis of Intermediate C190

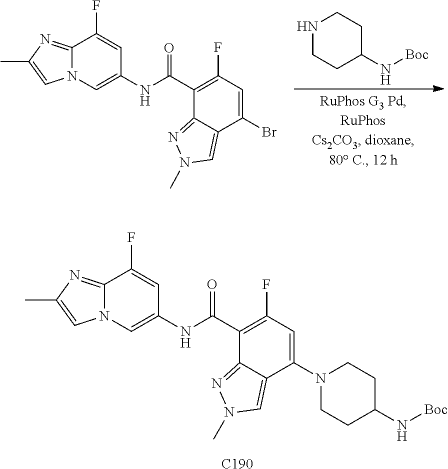 [SMILES: Cc1cn2cc(NC(=O)c3c(F)cc(Br)c4cn(C)nc34)cc(F)c2n1 ; CC(C)(C)OC(=O)NC1CCNCC1 ; Cc1cn2cc(NC(=O)c3c(F)cc(N4CCC(NC(=O)OC(C)(C)C)CC4)c4cn(C)nc34)cc(F)c2n1 ; "C190"]

To a stirred mixture of 4-bromo-N-{8-fluoro-2-methylimidazo[1,2-a]pyridin-6-yl}-2-methylindazole-7-carboxamide (60 mg, 0.149 mmol, 1 equiv) and tert-butyl N-(piperidin-4-yl)carbamate (35.85 mg, 0.179 mmol, 1.2 equiv) in dioxane (1 mL) was added Cs₂CO₃ (145.81 mg, 0.447 mmol, 3 equiv), RuPhos (13.92 mg, 0.030 mmol, 0.2 equiv) and RuPhos Palladacycle Gen.3 (12.48 mg, 0.015 mmol, 0.1 equiv) at room temperature under nitrogen atmosphere. The resulting mixture was stirred for 12 hr at 80° C. under nitrogen atmosphere. The resulting mixture was concentrated under reduced pressure. The residue was purified by silica gel column chromatography, eluted with CH₂Cl₂/MeOH (10:1) to afford tert-butyl N-{1-[7-({8-fluoro-2-methylimidazo[1,2-a]pyridin-6-yl}carbamoyl)-2-methylindazol-4-yl]piperidin-4-yl}carbamate (C190, 50 mg, 64%) as a solid. LCMS (ES, m/z): 522 [M+H]⁺

### Synthesis of Compound 402

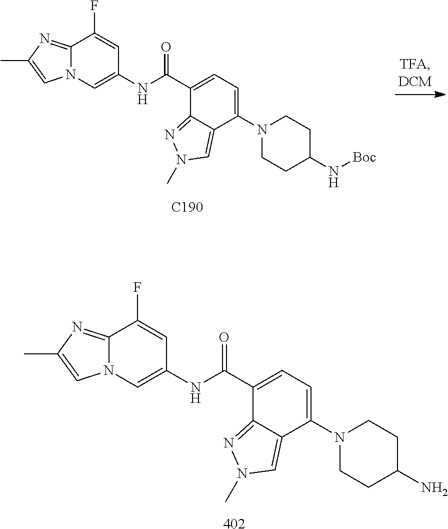

To a stirred solution of tert-butyl N-{1-[7-({8-fluoro-2-methylimidazo[1,2-a]pyridin-6-yl}carbamoyl)-2-methylindazol-4-yl]piperidin-4-yl}carbamate (50 mg, 0.096 mmol, 1 equiv) in DCM (1 mL) was added TFA (1 mL) at room temperature. The resulting mixture was stirred for 2 hr at room temperature. The resulting mixture was concentrated under reduced pressure. The crude product was purified by Prep-HPLC (Condition 10, Gradient 5) to afford 4-(4-aminopiperidin-1-yl)-N-{8-fluoro-2-methylimidazo[1,2-a]pyridin-6-yl}-2-methylindazole-7-carboxamide (Compound 402, 8.6 mg, 21%) as solid. LCMS (ES, m/z): 422 [M+H]^{+ 1}H NMR (400 MHz, DMSO-d₆) δ 11.07 (s, 1H), 9.21 (d, J=1.6 Hz, 1H), 8.77 (s, 1H), 7.96 (d, J=8.1 Hz, 1H), 7.90 (d, J=3.0 Hz, 1H), 7.34 (dd, J=12.4, 1.7 Hz, 1H), 6.49 (d, J=8.2 Hz, 1H), 4.30 (s, 3H), 3.89 (d, J=12.8 Hz, 2H), 3.05 (t, J=11.8 Hz, 2H), 2.85 (s, 1H), 2.35 (s, 3H), 1.88 (d, J=12.8 Hz, 2H), 1.43 (q, J=10.7, 10.3 Hz, 2H).

### Example 152: Synthesis of Compound 403

### Synthesis of Intermediate C191

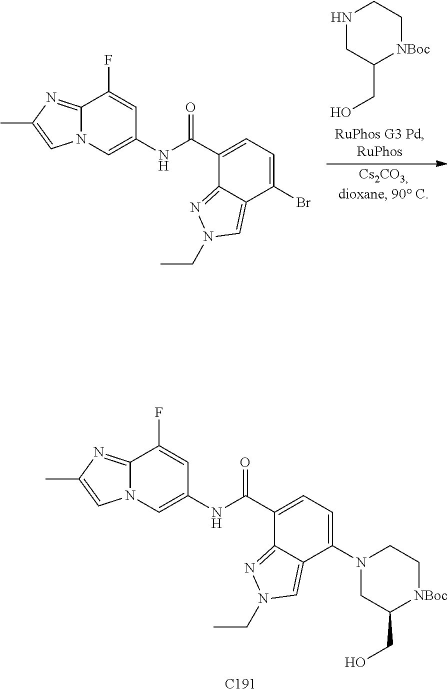 [SMILES: CCn1cc2c(Br)ccc(C(=O)Nc3cc(F)c4nc(C)cn4c3)c2n1 ; CC(C)(C)OC(=O)N1CCNCC1CO ; "Cs2CO3,dioxane, 90° C." ; CCn1cc2c(N3CCN(C(=O)OC(C)(C)C)[C@@H](CO)C3)ccc(C(=O)Nc3cc(F)c4nc(C)cn4c3)c2n1 ; "C191"]

To a solution of 4-bromo-2-ethyl-N-{8-fluoro-2-methylimidazo[1,2-a]pyridin-6-yl}indazole-7-carboxamide (1 g, 2.402 mmol, 1 equiv) and tert-butyl 2-(hydroxymethyl)piperazine-1-carboxylate (0.57 g, 2.642 mmol, 1.1 equiv) in dioxane (20 mL) were added Cs₂CO₃ (2.35 g, 7.206 mmol, 3.0 equiv), RuPhos Palladacycle Gen.3 (0.4 g, 0.480 mmol, 0.2 equiv) and RuPhos (0.22 g, 0.480 mmol, 0.2 equiv). After stirring for 3 hr at 90° C. under a nitrogen atmosphere. The resulting mixture was diluted with H₂O (20 mL). The resulting mixture was extracted with EA (3×20 mL). The combined organic layers were washed with NaCl (1×10 mL), dried over anhydrous Na₂SO₄. After filtration, the filtrate was concentrated under reduced pressure. The residue was purified by reverse flash chromatography (Condition 5, Gradient 2) to afford tert-butyl 4-[2-ethyl-7-({8-fluoro-2-methylimidazo[1,2-a]pyridin-6-yl}carbamoyl) indazol-4-yl]-2-(hydroxymethyl)piperazine-1-carboxylate (C191, 500 mg, 37%) as a solid. LCMS (ES, m/z): 552 [M+H]

### Synthesis of Compound 403

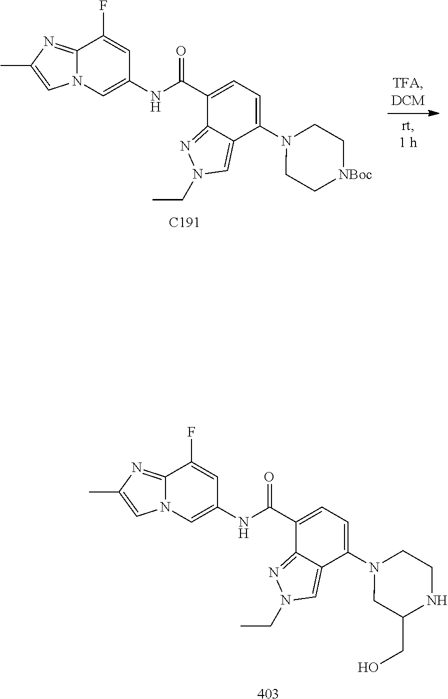 [SMILES: CCn1cc2c(N3CCN(C(=O)OC(C)(C)C)CC3)ccc(C(=O)Nc3cc(F)c4nc(C)cn4c3)c2n1 ; "C191" ; CCn1cc2c(N3CCNC(CO)C3)ccc(C(=O)Nc3cc(F)c4nc(C)cn4c3)c2n1 ; "403"]

A solution of tert-butyl 4-[2-ethyl-7-({8-fluoro-2-methylimidazo[1,2-a]pyridin-6-yl}carbamoyl) indazol-4-yl]-2-(hydroxymethyl)piperazine-1-carboxylate (50 mg, 0.091 mmol, 1 equiv) and TFA (0.2 mL) in DCM (2 mL) was stirred for 1 hr at room temperature. The mixture was neutralized to pH 8 with ammonia in methanol. The resulting mixture was concentrated under reduced pressure. The residue was purified by reverse flash chromatography (Condition 5, Gradient 3) to afford 2-ethyl-N-{8-fluoro-2-methylimidazo[1,2-a]pyridin-6-yl}-4-[3-(hydroxymethyl)piperazin-1-yl]indazole-7-carboxamide (Compound 403, 10 mg, 24%) as a solid.

LCMS (ES, m/z): 452 [M+H]^{+ 1}H NMR (300 MHz, DMSO-d₆) b 11.11 (s, 1H), 9.21 (d, J=1.7 Hz, 1H), 8.80 (s, 1H), 7.98 (d, J=8.1 Hz, 1H), 7.91 (dd, J=3.2, 1.0 Hz, 1H), 7.31 (dd, J=12.3, 1.7 Hz, 1H), 6.49 (d, J=8.2 Hz, 1H), 4.73 (s, 1H), 4.60 (q, J=7.3 Hz, 2H), 3.79 (d, J=10.5 Hz, 2H), 3.44 (s, 2H), 3.09-2.98 (m, 1H), 2.91 (q, J=10.3, 9.3 Hz, 3H), 2.77-2.63 (m, 1H), 2.39-2.32 (m, 3H), 1.62 (t, J=7.3 Hz, 3H).

### Example 153: Synthesis of Compound 404

### Synthesis of Intermediate 192

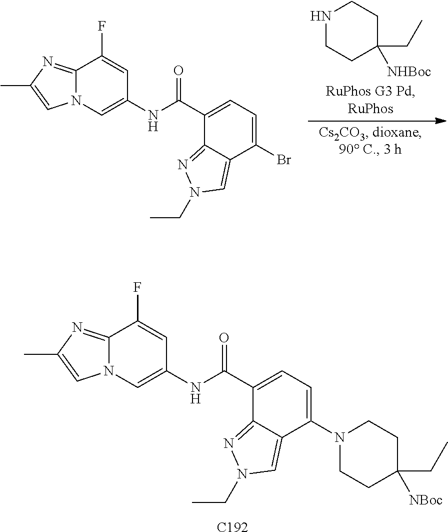

To a stirred solution of 4-bromo-2-ethyl-N-{8-fluoro-2-methylimidazo[1,2-a]pyridin-6-yl}indazole-7-carboxamide (172 mg, 0.413 mmol, 1 equiv) and tert-butyl N-(4-ethylpiperidin-4-yl)carbamate (94 mg, 0.413 mmol, 1 equiv) in dioxane (10 mL) were added Cs₂CO₃ (404 mg, 1.239 mmol, 3 equiv) and RuPhos Palladacycle Gen.3 (19 mg, 0.041 mmol, 0.1 equiv) in portions at room temperature under nitrogen atmosphere. The resulting mixture was stirred for 3 hr at 90° C. under nitrogen atmosphere. The mixture was allowed to cool down to room temperature. The resulting mixture was concentrated under vacuum. The residue was purified by silica gel column chromatography, eluted with PE/EA (1:5) to afford tert-butyl N-{4-ethyl-1-[2-ethyl-7-({8-fluoro-2-methylimidazo[1,2-a]pyridin-6-yl}carbamoyl) indazol-4-yl]piperidin-4-yl}carbamate (C192, 150 mg, 64%) as a solid. LCMS (ES, m/z): 564 [M+H]⁺

### Synthesis of Compound 404

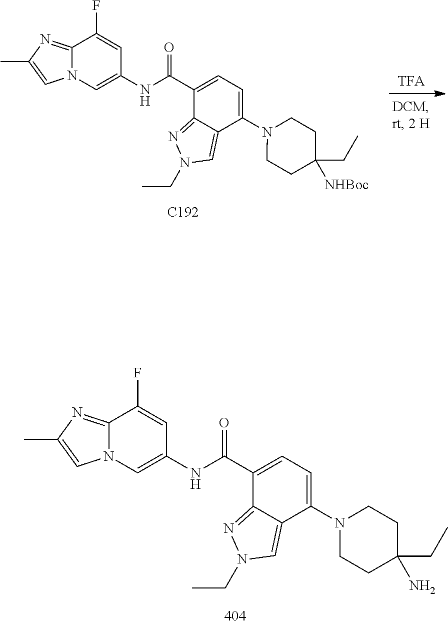

A solution of tert-butyl N-{4-ethyl-1-[2-ethyl-7-({8-fluoro-2-methylimidazo[1,2-a]pyridin-6-yl}carbamoyl) indazol-4-yl]piperidin-4-yl}carbamate (140 mg, 0.248 mmol, 1 equiv) in 2,2,2-trifluoroacetic acid (5 mL) and DCM (5 mL) was stirred for 2 h at room temperature. The resulting mixture was concentrated under reduced pressure. The residue was basified to pH 8 with 7 M NH₃ (g) in methanol. The resulting mixture was concentrated under vacuum. The residue was purified by reverse flash chromatography (Condition 3, Gradient 4) to afford 4-(4-amino-4-ethylpiperidin-1-yl)-2-ethyl-N-{8-fluoro-2-methylimidazo[1,2-a]pyridin-6-yl}indazole-7-carboxamide (Compound 402, 30 mg, 26%) as a solid. LCMS (ES, m/z): 464 [M+H]^{+ 1}H NMR (300 MHz, DMSO-d₆) δ 11.10 (s, 1H), 9.20 (d, J=1.6 Hz, 1H), 8.79 (s, 1H), 7.97 (d, J=8.1 Hz, 1H), 7.90 (d, J=3.1 Hz, 1H), 7.29 (dd, J=12.4, 1.7 Hz, 1H), 6.48 (d, J=8.2 Hz, 1H), 4.59 (q, J=7.3 Hz, 2H), 3.62 (dt, J=13.1, 4.2 Hz, 2H), 3.51-3.36 (m, 2H), 2.35 (s, 3H), 1.62 (t, J=7.2 Hz, 6H), 1.54-1.46 (m, 2H), 1.40 (q, J=7.5 Hz, 2H), 0.88 (t, J=7.4 Hz, 3H).

### Example 154: Synthesis of Compound 405

### Synthesis of Intermediate C193

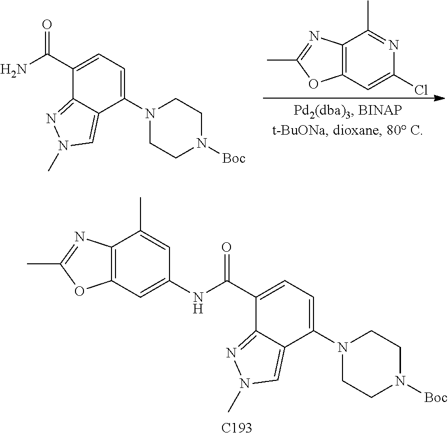 [SMILES: Cn1cc2c(N3CCN(C(=O)OC(C)(C)C)CC3)ccc(C(N)=O)c2n1 ; Cc1nc2c(C)nc(Cl)cc2o1 ; Cc1nc2c(C)cc(NC(=O)c3ccc(N4CCN(C(=O)OC(C)(C)C)CC4)c4cn(C)nc34)cc2o1 ; "C193"]

A solution of tert-butyl 4-(7-carbamoyl-2-methylindazol-4-yl)piperazine-1-carboxylate (35 mg, 0.097 mmol, 1 equiv), 6-chloro-2,4-dimethyl-[1,3]oxazolo[4,5-c]pyridine (21.34 mg, 0.116 mmol, 1.2 equiv), Pd₂(dba)₃ (8.92 mg, 0.010 mmol, 0.1 equiv), BINAP (12.13 mg, 0.019 mmol, 0.2 equiv) and tert-butoxysodium (28.08 mg, 0.291 mmol, 3 equiv) in dioxane (1 mL) was stirred for 3 hr at 80° C. under nitrogen atmosphere. The resulting mixture was extracted with DCM and water. The combined organic layers dried over anhydrous Na₂SO₄. After filtration, the filtrate was concentrated under reduced pressure. The residue was purified by silica gel column chromatography, eluted with PE:EA=3:1 to afford tert-butyl 4-[7-({2,4-dimethyl-[1,3]oxazolo[4,5-c]pyridin-6-yl}carbamoyl)-2-methylindazol-4-yl]piperazine-1-carboxylate (C193, 28 mg, 56%) as a solid. LCMS (ES, m/z): 524 [M+H]⁺

### Synthesis of Compound 405

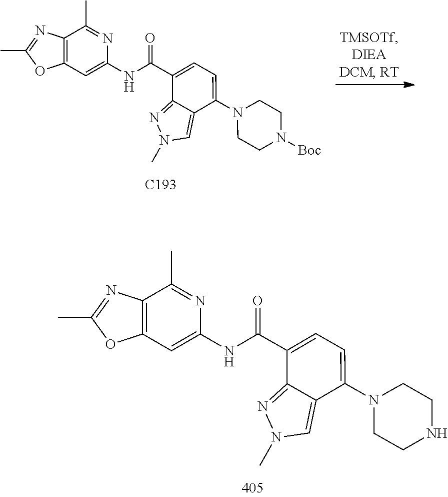

A solution of tert-butyl 4-[7-({2,4-dimethyl-[1,3]oxazolo[4,5-c]pyridin-6-yl}carbamoyl)-2-methylindazol-4-yl]piperazine-1-carboxylate (28 mg, 0.055 mmol, 1 equiv) in DCM (1 mL) was treated with DIEA (85.90 mg, 0.660 mmol, 12 equiv) and trimethylsilyl triflate (123.09 mg, 0.550 mmol, 10 equiv) for 1 h at room temperature. The mixture was basified to pH 9 with NaHCO₃. The resulting mixture was extracted with DCM and water. The combined organic layers were dried over anhydrous Na₂SO₄. After filtration, the filtrate was concentrated under reduced pressure. The residue was purified by Prep-TLC (Condition 1, Gradient 1) to afford N-{2,4-dimethyl-[1,3]oxazolo[4,5-c]pyridin-6-yl}-2-methyl-4-(piperazin-1-yl) indazole-7-carboxamide (Compound 405, 7.3 mg, 32%) as a solid. LCMS (ES, m/z): 406 [M+H] 1H NMR (300 MHz, DMSO-d6) δ 11.60 (s, 1H), 8.82 (s, 1H), 8.45 (s, 1H), 8.05 (d, J=8.1 Hz, 1H), 6.51 (d, J=8.3 Hz, 1H), 4.28 (s, 3H), 3.42-3.37 (m, 4H), 2.93 (t, J=5.0 Hz, 4H), 2.66 (s, 3H), 2.64 (s, 3H).

### Example 155: Synthesis of Compound 406

### Synthesis of Intermediate C194

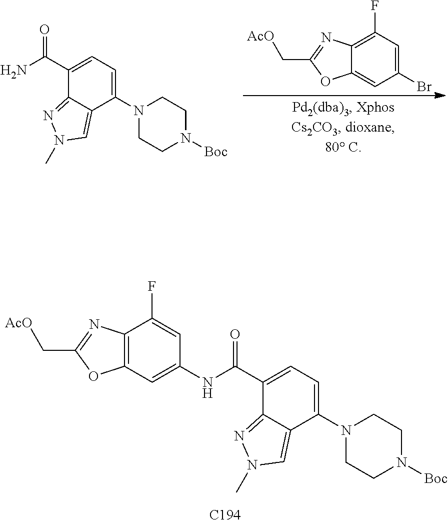 [SMILES: Cn1cc2c(N3CCN(C(=O)OC(C)(C)C)CC3)ccc(C(N)=O)c2n1 ; CC(=O)OCc1nc2c(F)cc(Br)cc2o1 ; CC(=O)OCc1nc2c(F)cc(NC(=O)c3ccc(N4CCN(C(=O)OC(C)(C)C)CC4)c4cn(C)nc34)cc2o1 ; "C194"]

A solution of (6-bromo-4-fluoro-1,3-benzoxazol-2-yl)methyl acetate (100 mg, 0.347 mmol, 1 equiv), tert-butyl 4-(7-carbamoyl-2-methylindazol-4-yl)piperazine-1-carboxylate (149.73 mg, 0.416 mmol, 1.2 equiv), Pd₂(dba)₃ (31.79 mg, 0.035 mmol, 0.1 equiv), Xantphos (40.17 mg, 0.069 mmol, 0.2 equiv) and caesio methaneperoxoate caesium (340.36 mg, 1.041 mmol, 3 equiv) in dioxane (2 mL) was stirred for 4 hr at 80° C. under nitrogen atmosphere. The resulting mixture was extracted with DCM and water. The combined organic layers were dried over anhydrous Na₂SO₄. After filtration, the filtrate was concentrated under reduced pressure. The residue was purified by silica gel column chromatography, eluted with PE:EA=3:1 to afford tert-butyl 4-[7-({2-[(acetyloxy)methyl]-4-fluoro-1,3-benzoxazol-6-yl}carbamoyl)-2-methylindazol-4-yl]piperazine-1-carboxylate (C194, 83 mg, 42%) as a solid. LCMS (ES, m/z): 567 [M+H]⁺

### Synthesis of Intermediate C195

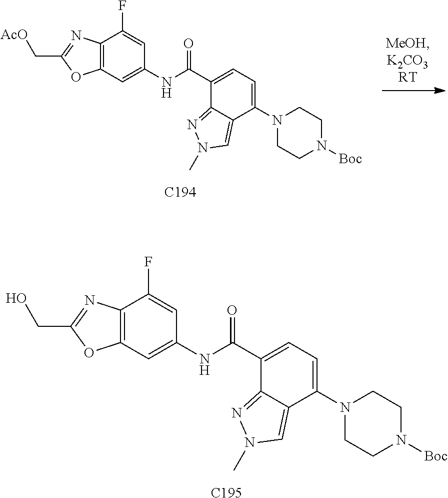 [SMILES: CC(=O)OCc1nc2c(F)cc(NC(=O)c3ccc(N4CCN(C(=O)OC(C)(C)C)CC4)c4cn(C)nc34)cc2o1 ; "C194" ; Cn1cc2c(N3CCN(C(=O)OC(C)(C)C)CC3)ccc(C(=O)Nc3cc(F)c4nc(CO)oc4c3)c2n1 ; "C195"]

A solution of tert-butyl 4-[7-({2-[(acetyloxy)methyl]-4-fluoro-1,3-benzoxazol-6-yl}carbamoyl)-2-methylindazol-4-yl]piperazine-1-carboxylate (63 mg, 0.111 mmol, 1 equiv) in methanol (2 mL) was treated with potassium methaneperoxoate potassium (46.44 mg, 0.333 mmol, 3 equiv) for 2 hr at room temperature. The resulting mixture was filtered, the filter cake was washed with DCM. The filtrate was concentrated under reduced pressure. The residue was purified by silica gel column chromatography, eluted with PE:EA=1:1 to afford tert-butyl 4-(7-{[4-fluoro-2-(hydroxymethyl)-1,3-benzoxazol-6-yl]carbamoyl}-2-methylindazol-4-yl)piperazine-1-carboxylate (C195, 42 mg, 72%) as a solid. LCMS (ES, m/z): 525 [M+H]⁺

### Synthesis of Compound 406

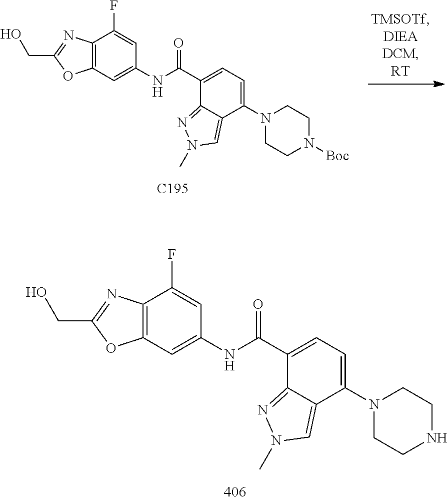 [SMILES: Cn1cc2c(N3CCN(C(=O)OC(C)(C)C)CC3)ccc(C(=O)Nc3cc(F)c4nc(CO)oc4c3)c2n1 ; "C195" ; Cn1cc2c(N3CCNCC3)ccc(C(=O)Nc3cc(F)c4nc(CO)oc4c3)c2n1 ; "406"]

A solution of tert-butyl 4-(7-{[4-fluoro-2-(hydroxymethyl)-1,3-benzoxazol-6-yl]carbamoyl}-2-methylindazol-4-yl)piperazine-1-carboxylate (42 mg, 0.080 mmol, 1 equiv) in DCM (1 mL) was treated with DIEA (124.18 mg, 0.960 mmol, 12 equiv), trimethylsilyl triflate (177.95 mg, 0.800 mmol, 10 equiv) for 1 hr at room temperature. The mixture was basified to pH 9 with NaHCO₃. The resulting mixture was extracted with DCM and water. The combined organic layers were dried over anhydrous Na₂SO₄. After filtration, the filtrate was concentrated under reduced pressure. The residue was purified by Prep-TLC (Condition 1, Gradient 1) to afford N-[4-fluoro-2-(hydroxymethyl)-1,3-benzoxazol-6-yl]-2-methyl-4-(piperazin-1-yl) indazole-7-carboxamide (Compound 406, 8.1 mg, 23%) as a solid. LCMS (ES, m/z): 425 [M+H]⁺1H NMR (300 MHz, DMSO-d6) δ 11.47 (s, 1H), 8.80 (s, 1H), 8.19 (d, J=1.6 Hz, 1H), 8.00 (d, J=8.1 Hz, 1H), 7.66 (dd, J=12.1, 1.7 Hz, 1H), 6.49 (d, J=8.2 Hz, 1H), 5.95 (s, 1H), 4.71 (s, 2H), 4.30 (s, 3H), 2.91 (t, J=5.0 Hz, 4H), 2.51 (t, J=3.7 Hz, 4H).

### Example 156: Synthesis of Compounds 407 and 408

### Synthesis of Intermediate C196

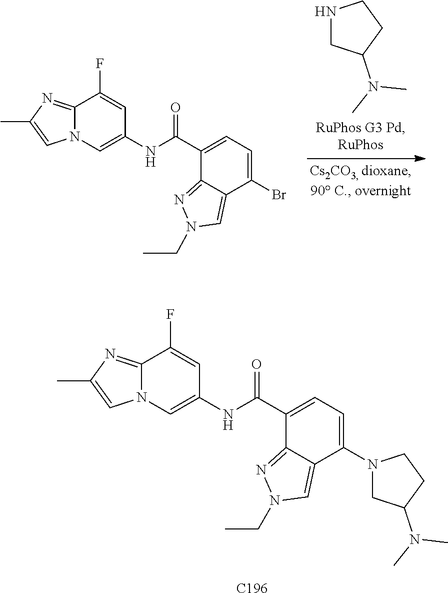

To a stirred solution of 4-bromo-2-ethyl-N-{8-fluoro-2-methylimidazo[1,2-a]pyridin-6-yl}indazole-7-carboxamide (90 mg, 0.22 mmol, 1 equiv) and N,N-dimethylpyrrolidin-3-amine (25 mg, 0.22 mmol, 1 equiv) in dioxane (5 mL) were added Cs₂CO₃ (211 mg, 0.66 mmol, 3 equiv), RuPhos (20 mg, 0.044 mmol, 0.2 equiv) and RuPhos Palladacycle Gen.3 (18 mg, 0.022 mmol, 0.1 equiv) in portions at room temperature under nitrogen atmosphere. The resulting mixture was stirred for overnight at 90° C. under nitrogen atmosphere. The mixture was allowed to cool down to room temperature. The resulting mixture was concentrated under reduced pressure. The residue was dissolved in water (20 mL). The resulting mixture was extracted with ethyl acetate (3×5 mL). The combined organic layers were washed with brine (1×10 mL), dried over anhydrous Na₂SO₄. After filtration, the filtrate was concentrated under reduced pressure. The residue was purified by prep-HPLC (Condition 12, Gradient 2) to afford 4-[3-(dimethylamino)pyrrolidin-1-yl]-2-ethyl-N-{8-fluoro-2-methylimidazo[1,2-a]pyridin-6-yl}indazole-7-carboxamide (70 mg, 71%) as a solid. LCMS (ES, m/z): 450 [M+H]^{+ 1}H NMR (300 MHz, DMSO-d₆) δ 11.06 (s, 1H), 9.19 (d, J=1.6 Hz, 1H), 8.88 (s, 1H), 7.98-7.86 (m, 2H), 7.27 (dd, J=12.4, 1.7 Hz, 1H), 6.05 (d, J=8.4 Hz, 1H), 4.57 (q, J=7.3 Hz, 2H), 3.81 (dt, J=19.0, 9.0 Hz, 2H), 3.65 (q, J=9.5, 9.0 Hz, 1H), 3.46 (t, J=9.0 Hz, 1H), 2.91 (s, 1H), 2.35 (s, 3H), 2.28 (s, 6H), 2.23 (s, 1H), 1.93 (q, J=11.6, 10.7 Hz, 1H), 1.61 (t, J=7.2 Hz, 3H).

### Synthesis of Compounds 407 and 408

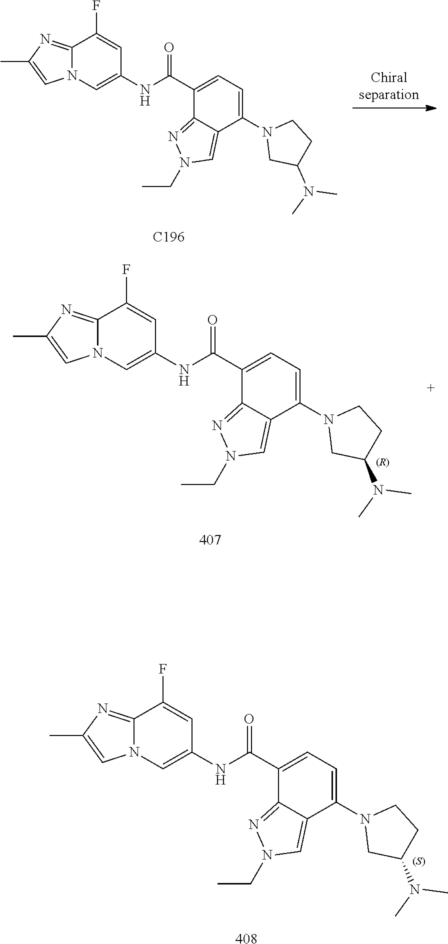

C196 was separated by Prep-Chiral-HPLC (Condition 4, Gradient 1) to afford (R)-4-(3-(dimethylamino)pyrrolidin-1-yl)-2-ethyl-N-(8-fluoro-2-methylimidazo[1,2-a]pyridin-6-yl)-2H-indazole-7-carboxamide (Compound 407, 23.9 mg, 36%) as a white solid and (S)-4-(3-(dimethylamino)pyrrolidin-1-yl)-2-ethyl-N-(8-fluoro-2-methylimidazo[1,2-a]pyridin-6-yl)-2H-indazole-7-carboxamide (Compound 408, 25.7 mg, 39%) as a solid.

| Compound | |
|---|---|
| No. | Analysis Data |
| 407 | LCMS (ES, m/z): 450 [M + H]⁺ |
| | ¹H NMR (300 MHz, DMSO-d₆) δ 11.06 (s, 1H), 9.19 (d, J = 1.6 Hz, 1H), 8.88 (s, |
| | 1H), 7.98-7.86 (m, 2H), 7.27 (dd, J = 12.4, 1.7 Hz, 1H), 6.05 (d, J = 8.4 Hz, 1H), 4.57 |
| | (q, J = 7.3 Hz, 2H), 3.81 (dt, J = 19.0, 9.0 Hz, 2H), 3.65 (q, J = 9.5, 9.0 Hz, 1H), 3.46 |
| | (t, J = 9.0 Hz, 1H), 2.91 (s, 1H), 2.35 (s, 3H), 2.28 (s, 6H), 2.23 (s, 1H), 1.93 (q, J = |
| | 11.6, 10.7 Hz, 1H), 1.61 (t, J = 7.2 Hz, 3H). |
| 408 | LCMS (ES, m/z): 450 [M + H]⁺ |
| | ¹H NMR (300 MHz, DMSO-d₆) δ 11.06 (s, 1H), 9.19 (d, J = 1.6 Hz, 1H), 8.88 (s, |
| | 1H), 7.98-7.86 (m, 2H), 7.27 (dd, J = 12.4, 1.7 Hz, 1H), 6.05 (d, J = 8.4 Hz, 1H), 4.57 |
| | (q, J = 7.3 Hz, 2H), 3.81 (dt, J = 19.0, 9.0 Hz, 2H), 3.65 (q, J = 9.5, 9.0 Hz, 1H), 3.46 |
| | (t, J = 9.0 Hz, 1H), 2.91 (s, 1H), 2.35 (s, 3H), 2.28 (s, 6H), 2.23 (s, 1H), 1.93 (q, J = |
| | 11.6, 10.7 Hz, 1H), 1.61 (t, J = 7.2 Hz, 3H). |

### Example 157: Synthesis of Compounds 398, 409, and 410

### Synthesis of Intermediate C197

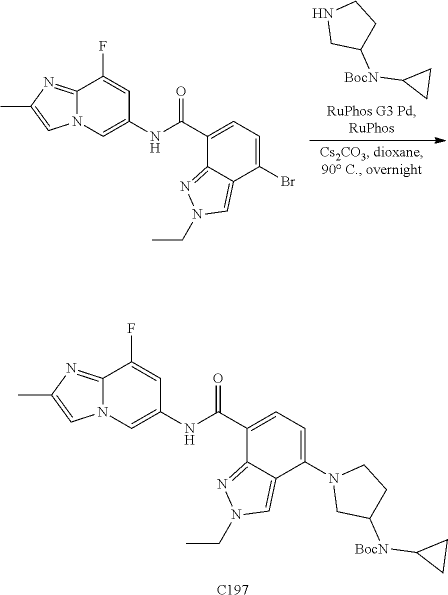 [SMILES: CCn1cc2c(Br)ccc(C(=O)Nc3cc(F)c4nc(C)cn4c3)c2n1 ; CC(C)(C)OC(=O)N(C1CC1)C1CCNC1 ; CCn1cc2c(N3CCC(N(C(=O)OC(C)(C)C)C4CC4)C3)ccc(C(=O)Nc3cc(F)c4nc(C)cn4c3)c2n1 ; "C197"]

To a stirred solution of 4-bromo-2-ethyl-N-{8-fluoro-2-methylimidazo[1,2-a]pyridin-6-yl}indazole-7-carboxamide (190 mg, 0.456 mmol, 1 equiv) and tert-butyl N-cyclopropyl-N-(pyrrolidin-3-yl)carbamate (103 mg, 0.456 mmol, 1 equiv) in dioxane (10 mL) were added Cs₂CO₃ (446 mg, 1.368 mmol, 3 equiv), RuPhos (43 mg, 0.091 mmol, 0.2 equiv) and RuPhos Palladacycle Gen.3 (21 mg, 0.046 mmol, 0.1 equiv) in portions at room temperature under nitrogen atmosphere. The resulting mixture was stirred for overnight at 90° C. under nitrogen atmosphere. The mixture was allowed to cool down to room temperature. The resulting mixture was concentrated under reduced pressure. The residue was purified by reverse flash chromatography (Condition 5, Gradient 2) to afford tert-butyl N-cyclopropyl-N-{1-[2-ethyl-7-({8-fluoro-2-methylimidazo[1,2-a]pyridin-6-yl}carbamoyl) indazol-4-yl]pyrrolidin-3-yl}carbamate (C197, 200 mg, 78%) as a solid. LCMS (ES, m/z): 562 [M+H]⁺

### Synthesis of Compound 398

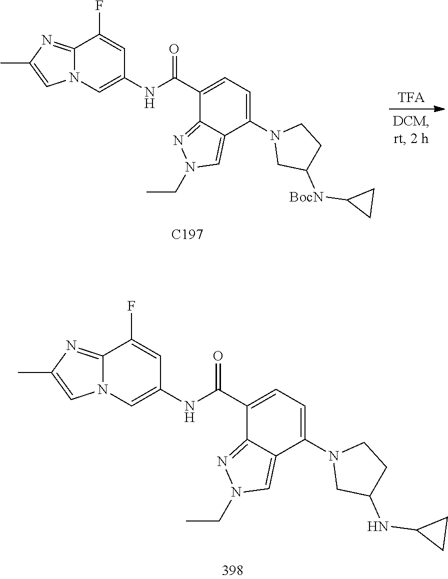 [SMILES: CCn1cc2c(N3CCC(N(C(=O)OC(C)(C)C)C4CC4)C3)ccc(C(=O)Nc3cc(F)c4nc(C)cn4c3)c2n1 ; "C197" ; CCn1cc2c(N3CCC(NC4CC4)C3)ccc(C(=O)Nc3cc(F)c4nc(C)cn4c3)c2n1]

A solution of tert-butyl N-cyclopropyl-N-{1-[2-ethyl-7-({8-fluoro-2-methylimidazo[1,2-a]pyridin-6-yl}carbamoyl) indazol-4-yl]pyrrolidin-3-yl}carbamate (90 mg, 0.160 mmol, 1 equiv) in trifluoroacetic acid (2 mL) and DCM (2 mL) was stirred for 2 h at room temperature. The resulting mixture was concentrated under reduced pressure. The residue was basified to pH 8 with 7 M NH₃(g) in MeOH. The resulting mixture was concentrated under reduced pressure. The residue was purified by prep-HPLC (Condition 10, Gradient 4) to afford 4-[3-(cyclopropylamino)pyrrolidin-1-yl]-2-ethyl-N-{8-fluoro-2-methylimidazo[1,2-a]pyridin-6-yl}indazole-7-carboxamide (Compound 398, 60 mg, 81.13%) as a solid. LCMS (ES, m/z): 462 [M+H]^{+ 1}H NMR (300 MHz, DMSO-d₆) δ 11.04 (s, 1H), 9.19 (d, J=1.6 Hz, 1H), 8.83 (s, 1H), 7.97-7.85 (m, 2H), 7.25 (dd, J=12.4, 1.7 Hz, 1H), 6.00 (d, J=8.4 Hz, 1H), 4.57 (q, J=7.3 Hz, 2H), 3.77 (dd, J=12.8, 6.6 Hz, 2H), 3.69-3.41 (m, 3H), 2.71 (m, 1H) 2.35 (s, 3H), 2.17 (td, J=7.1, 6.5, 3.9 Hz, 2H), 1.98 (dd, J=12.3, 6.2 Hz, 1H), 1.61 (t, J=7.2 Hz, 3H), 0.42 (d, J=6.6 Hz, 2H), 0.25 (dq, J=9.7, 6.2, 4.7 Hz, 2H).

### Synthesis of Compounds 409 and 410

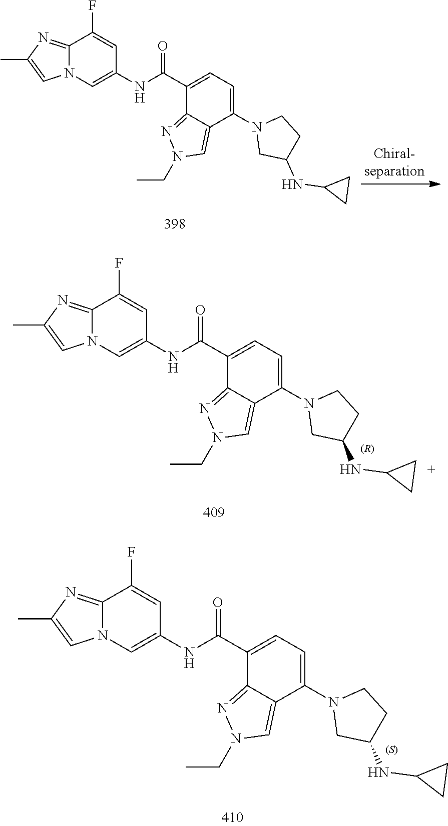

Compound 398 was chiral-separation by Prep-Chiral-HPLC (Condition 5, Gradient 1) to afford (R)-4-(3-(cyclopropylamino)pyrrolidin-1-yl)-2-ethyl-N-(8-fluoro-2-methylimidazo[1,2-a]pyridin-6-yl)-2H-indazole-7-carboxamide (Compound 409, 20 mg, 35%) as a solid and (S)-4-(3-(cyclopropylamino)pyrrolidin-1-yl)-2-ethyl-N-(8-fluoro-2-methylimidazo[1,2-a]pyridin-6-yl)-2H-indazole-7-carboxamide (Compound 410, 18 mg, 31%) as a solid.

| Compound No. | Analysis Data |
|---|---|
| 409 | LCMS (ES, m/z): 462 [M + H]⁺ |
| | ¹H NMR (300 MHz, DMSO-d₆) δ 11.04 (s, 1H), 9.19 (d, J = 1.6 Hz, 1H), 8.83 (s, |
| | 1H), 7.97-7.85 (m, 2H), 7.25 (dd, J = 12.4, 1.7 Hz, 1H), 6.00 (d, J = 8.4 Hz, 1H), |
| | 4.57 (q, J = 7.3 Hz, 2H), 3.77 (dd, J = 12.8, 6.6 Hz, 2H), 3.69-3.41 (m, 3H), 2.71 |
| | (m, 1H)2.35 (s, 3H), 2.17 (td, J = 7.1, 6.5, 3.9 Hz, 2H), 1.98 (dd, J = 12.3, 6.2 Hz, |
| | 1H), 1.61 (t, J = 7.2 Hz, 3H), 0.42 (d, J = 6.6 Hz, 2H), 0.25 (dq, J = 9.7, 6.2, 4.7 Hz, |
| | 2H). |
| 410 | LCMS (ES, m/z): 462 [M + H]⁺ |
| | ¹H NMR (300 MHz, DMSO-d₆) δ 11.04 (s, 1H), 9.19 (d, J = 1.6 Hz, 1H), 8.83 (s, |
| | 1H), 7.97-7.85 (m, 2H), 7.25 (dd, J = 12.4, 1.7 Hz, 1H), 6.00 (d, J = 8.4 Hz, 1H), |
| | 4.57 (q, J = 7.3 Hz, 2H), 3.77 (dd, J = 12.8, 6.6 Hz, 2H), 3.69-3.41 (m, 3H), 2.71 |
| | (m, 1H)2.35 (s, 3H), 2.17 (td, J = 7.1, 6.5, 3.9 Hz, 2H), 1.98 (dd, J = 12.3, 6.2 Hz, |
| | 1H), 1.61 (t, J = 7.2 Hz, 3H), 0.42 (d, J = 6.6 Hz, 2H), 0.25 (dq, J = 9.7, 6.2, 4.7 Hz, |
| | 2H). |

### Example 158: Synthesis of Compounds 411 and 412

### Synthesis of Intermediate C198

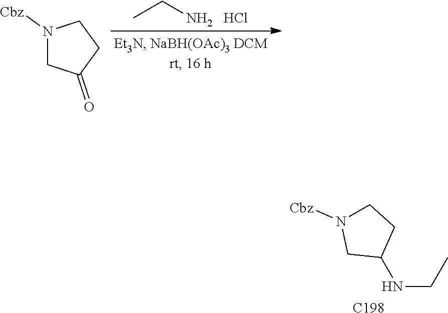

To a stirred solution of ethanamine hydrochloride (22.32 g, 273.672 mmol, 3 equiv) in DCM (300 mL) was added triethyl amine (27.69 g, 273.672 mmol, 3.0 equiv) at room temperature. The mixture was stirred for 10 min at room temperature. To the above mixture was added benzyl 3-oxopyrrolidine-1-carboxylate (20 g, 91.224 mmol, 1.0 equiv), NaBH(OAc)₃ (29.00 g, 136.836 mmol, 1.5 equiv) in portions over 10 min at 0° C. The resulting mixture was stirred for additional 16 hr at room temperature. The reaction was quenched with water at 0° C. and diluted with water (200 mL). The resulting mixture was extracted with CH₂Cl₂ (3×200 mL). The combined organic layers were washed with water (3×200 mL), dried over anhydrous Na₂SO₄. After filtration, the filtrate was concentrated under reduced pressure to afford benzyl 3-(ethylamino)pyrrolidine-1-carboxylate (C198, 47 g, 99%) as an oil. LCMS (ES, m/z): 249 [M+H]

### Synthesis of Intermediate C199

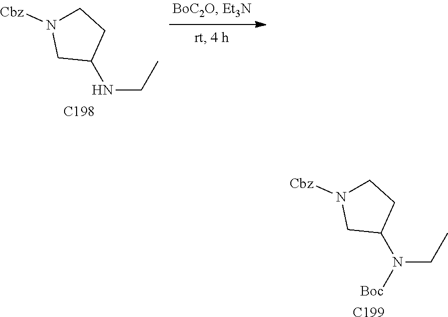 [SMILES: CCNC1CCN(C(=O)OCc2ccccc2)C1 ; "C198" ; CCN(C(=O)OC(C)(C)C)C1CCN(C(=O)OCc2ccccc2)C1 ; "C199"]

To a stirred mixture of benzyl 3-(ethylamino)pyrrolidine-1-carboxylate (47.00 g, 189.267 mmol, 1 equiv) in DCM (940 mL) were added Et₃N (57.46 g, 567.801 mmol, 3 equiv) and Boc₂O (61.96 g, 283.900 mmol, 1.5 equiv) in portions at room temperature. The resulting mixture was stirred for 4 h at room temperature. The resulting mixture was diluted with water (900 mL). The resulting mixture was extracted with CH₂Cl₂ (3×500 mL). The combined organic layers were washed with water (2×400 mL), dried over anhydrous Na₂SO₄. After filtration, the filtrate was concentrated under reduced pressure. The residue was purified by silica gel column chromatography, eluted with PE/EA (1:1) to afford benzyl 3-[(tert-butoxycarbonyl)(ethyl)amino]pyrrolidine-1-carboxylate (C199, 27 mg, 40%) as an oil. LCMS (ES, m/z): 349 [M+H]⁺

### Synthesis of Intermediate C200

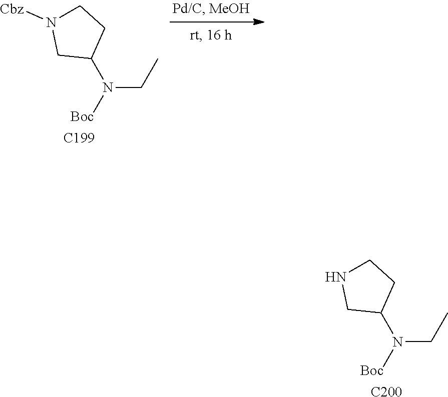 [SMILES: CCN(C(=O)OC(C)(C)C)C1CCN(C(=O)OCc2ccccc2)C1 ; "C199" ; CCN(C(=O)OC(C)(C)C)C1CCNC1 ; "C200"]

To a solution of benzyl 3-[(tert-butoxycarbonyl)(ethyl)amino]pyrrolidine-1-carboxylate (10 g, 28.699 mmol, 1 equiv) in methanol (100 mL) was added Pd/C (2 g 20% W) in a pressure tank. The mixture was hydrogenated at room temperature under 30 psi of hydrogen pressure for 16 hr. The resulting mixture was filtered and the precipitated solids was washed with MeOH (3×50 mL). The combined filtrate was concentrated under vacuum to afford tert-butyl N-ethyl-N-(pyrrolidin-3-yl)carbamate (C200, 5.1 g, 82%) as an oil. LCMS (ES, m/z): 214 [M+H]⁺ Synthesis of Intermediate C201

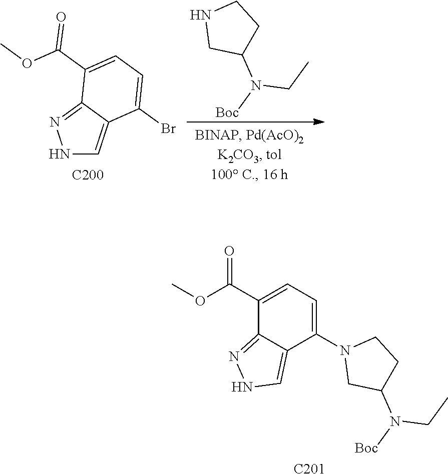 [SMILES: COC(=O)c1ccc(Br)c2c[nH]nc12 ; CCN(C(=O)OC(C)(C)C)C1CCNC1 ; "C200" ; CCN(C(=O)OC(C)(C)C)C1CCN(c2ccc(C(=O)OC)c3n[nH]cc23)C1 ; "C201"]

To a stirred mixture of methyl 4-bromo-2H-indazole-7-carboxylate (2.5 g, 9.801 mmol, 1 equiv) and tert-butyl N-ethyl-N-(pyrrolidin-3-yl)carbamate (3.15 g, 14.701 mmol, 1.5 equiv) in toluene (50 mL) were added K₂CO₃ (4.06 g, 29.403 mmol, 3 equiv) and BINAP (1.22 g, 1.960 mmol, 0.2 equiv) and Pd(AcO)₂ (0.22 g, 0.980 mmol, 0.1 equiv) at room temperature under nitrogen atmosphere. The resulting mixture was stirred for 16 hr at 100° C. under nitrogen atmosphere. The resulting mixture was concentrated under vacuum. The residue was purified by silica gel column chromatography, eluted with PE/EA (2:1) to afford methyl 4-{3-[(tert-butoxycarbonyl)(ethyl)amino]pyrrolidin-1-yl}-2H-indazole-7-carboxylate (C201, 3.2 g, 84%) as a solid. LCMS (ES, m/z): 389 [M+H]⁺

### Synthesis of Intermediate C202

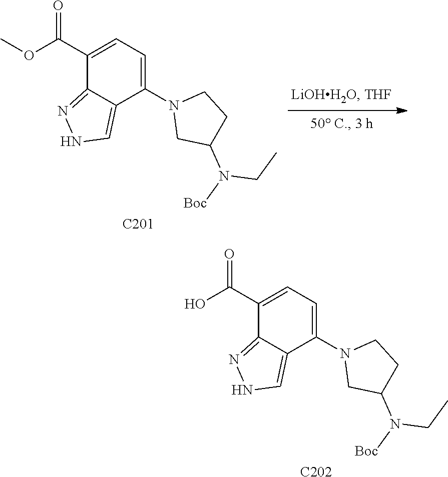 [SMILES: CCN(C(=O)OC(C)(C)C)C1CCN(c2ccc(C(=O)OC)c3n[nH]cc23)C1 ; "C201" ; CCN(C(=O)OC(C)(C)C)C1CCN(c2ccc(C(=O)O)c3n[nH]cc23)C1 ; "C202"]

To a stirred mixture of methyl 4-{3-[(tert-butoxycarbonyl)(ethyl)amino]pyrrolidin-1-yl}-2H-indazole-7-carboxylate (3.2 g, 8.237 mmol, 1 equiv) in THF (32 mL) were added H₂O (32 mL) and LiOH·H₂O (1.58 g, 65.896 mmol, 8 equiv) at room temperature. The resulting mixture was stirred for 3 hr at 50° C. The mixture was acidified to pH 6 with 1 N of HCl. The resulting mixture was extracted with ethyl acetate (4×40 mL). The combined organic layers were washed with water (3×30 mL), dried over anhydrous Na₂SO₄. After filtration, the filtrate was concentrated under reduced pressure to afford 4-{3-[(tert-butoxycarbonyl) (ethyl)amino]pyrrolidin-1-yl}-2H-indazole-7-carboxylic acid (C202, 2.48 g, 80%) as a solid. LCMS (ES, m/z): 375 [M+H]⁺

### Synthesis of Intermediate C203

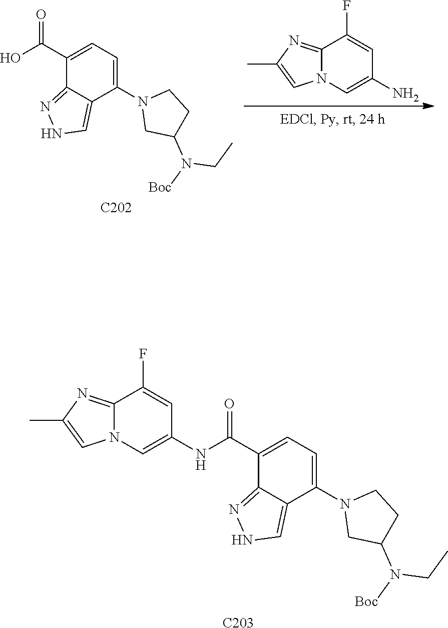 [SMILES: CCN(C(=O)OC(C)(C)C)C1CCN(c2ccc(C(=O)O)c3n[nH]cc23)C1 ; Cc1cn2cc(N)cc(F)c2n1 ; "C202" ; CCN(C(=O)OC(C)(C)C)C1CCN(c2ccc(C(=O)Nc3cc(F)c4nc(C)cn4c3)c3n[nH]cc23)C1 ; "C203"]

To a stirred mixture of 4-{3-[(tert-butoxycarbonyl)(ethyl)amino]pyrrolidin-1-yl}-2H-indazole-7-carboxylic acid (1.8 g, 4.807 mmol, 1.00 equiv) and 8-fluoro-2-methylimidazo[1,2-a]pyridin-6-amine (0.95 g, 5.768 mmol, 1.2 equiv) in pyridine (36 mL) was added EDCI (1.38 g, 7.211 mmol, 1.5 equiv) in portions at room temperature. The resulting mixture was stirred for 16 hr at room temperature. The resulting mixture was diluted with water (60 mL). The resulting mixture was extracted with ethyl acetate (3×60 mL). The combined organic layers were washed with water (3×50 mL), dried over anhydrous Na₂SO₄. After filtration, the filtrate was concentrated under reduced pressure. The residue was purified by silica gel column chromatography, eluted with PE/EA (1:1) to afford tert-butyl N-ethyl-N-{1-[7-({8-fluoro-2-methylimidazo[1,2-a]pyridin-6-yl}carbamoyl)-2H-indazol-4-yl]pyrrolidin-3-yl}carbamate (C203, 420 mg, 16%) as a solid. LCMS (ES, m/z): 522 [M+H]⁺

### Synthesis of Intermediate C203

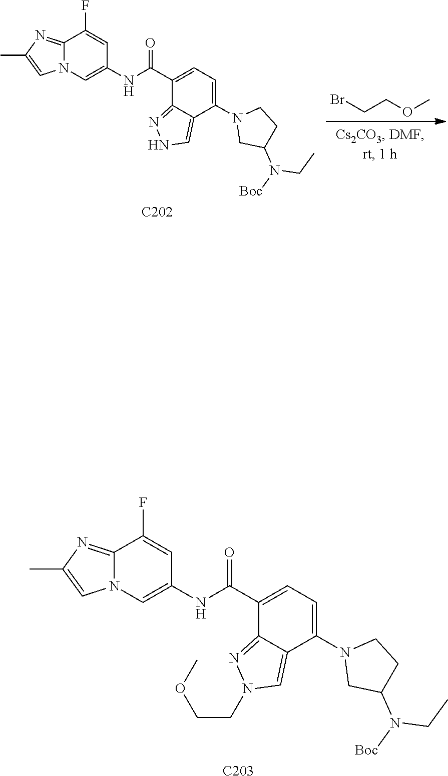

To a stirred mixture of tert-butyl N-ethyl-N-{1-[7-({8-fluoro-2-methylimidazo[1,2-a]pyridin-6-yl}carbamoyl)-2H-indazol-4-yl]pyrrolidin-3-yl}carbamate (400 mg, 0.767 mmol, 1 equiv) and 2-bromoethyl methyl ether (159.88 mg, 1.151 mmol, 1.5 equiv) in DMF (8 mL, 103.372 mmol, 134.80 equiv) was added Cs₂CO₃ (749.59 mg, 2.301 mmol, 3 equiv) at room temperature. The resulting mixture was stirred for 1 hr at room temperature. The resulting mixture was diluted with water (20 mL). The resulting mixture was extracted with ethyl acetate (3×20 mL). The combined organic layers were washed with water (3×20 mL), dried over anhydrous Na₂SO₄. After filtration, the filtrate was concentrated under reduced pressure. The residue was purified by silica gel column chromatography, eluted with EA to afford tert-butyl N-ethyl-N-{1-[7-({8-fluoro-2-methylimidazo[1,2-a]pyridin-6-yl}carbamoyl)-2-(2-methoxyethyl) indazol-4-yl]pyrrolidin-3-yl}carbamate (210 mg, 47.24%) as a solid. LCMS (ES, m/z): 580[M+H]⁺

### Synthesis of Intermediates C204 and C205

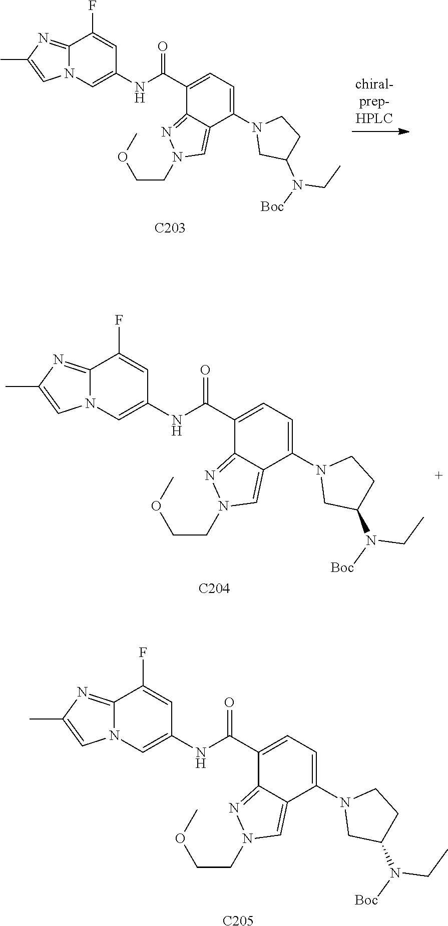

Intermediate C203 was separated by Chiral-Prep HPLC (Condition 4, Gradient 1) to yield intermediates C204 and C205.

| Intermediate No. | Analysis Data |
|---|---|
| C204 | RT = 8.054 min on chiral-HPLC |
| | LCMS (ES, m/z): 422 [M + H]⁺ |
| C205 | RT = 9.404 min on chiral-HPLC |
| | LCMS (ES, m/z): 422 [M + H]⁺ |

### Synthesis of Compound 412

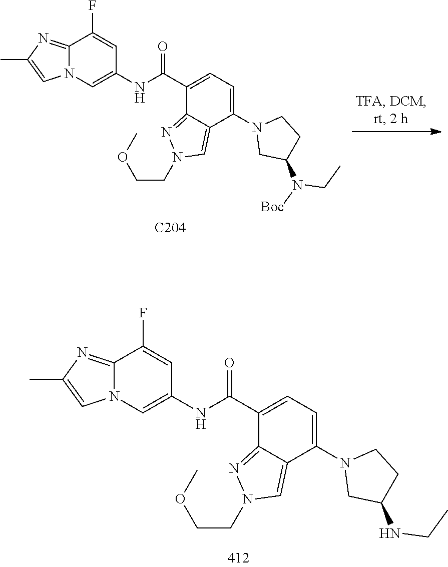

To a stirred mixture of tert-butyl N-ethyl-N-[(3R)-1-[7-({8-fluoro-2-methylimidazo[1,2-a]pyridin-6-yl}carbamoyl)-2-(2-methoxyethyl) indazol-4-yl]pyrrolidin-3-yl]carbamate (65 mg, 0.112 mmol, 1 equiv) in DCM (2 mL) was added TFA (0.4 mL) dropwise at room temperature. The resulting mixture was stirred for 2 hr at room temperature. The resulting mixture was concentrated under reduced pressure. The crude product was purified by Prep-HPLC (Condition 10, Gradient 2) to afford 4-[(3R)-3-(ethylamino)pyrrolidin-1-yl]-N-{8-fluoro-2-methylimidazo[1,2-a]pyridin-6-yl}-2-(2-methoxyethyl) indazole-7-carboxamide (Compound 412, 23.8 mg, 44%) as a solid. LCMS (ES, m/z): 422 [M+H]^{+ 1}H NMR (400 MHz, DMSO-d₆) δ 11.05 (s, 1H), 9.21 (d, J=1.6 Hz, 1H), 8.82 (s, 1H), 7.94 (d, J=8.3 Hz, 1H), 7.89 (d, J=3.1 Hz, 1H), 7.28 (dd, J=12.4, 1.7 Hz, 1H), 6.02 (d, J=8.4 Hz, 1H), 4.71 (t, J=5.2 Hz, 2H), 3.94 (t, J=5.2 Hz, 2H), 3.83-3.73 (m, 2H), 3.64 (d, J=8.0 Hz, 1H), 3.52-3.42 (m, 2H), 3.32 (s, 3H), 2.65 (q, J=7.0 Hz, 2H), 2.35 (s, 3H), 2.18 (dd, J=12.6, 6.3 Hz, 1H), 1.93 (dt, J=12.3, 6.3 Hz, 1H), 1.06 (t, J=7.1 Hz, 3H).

### Synthesis of Compound 411

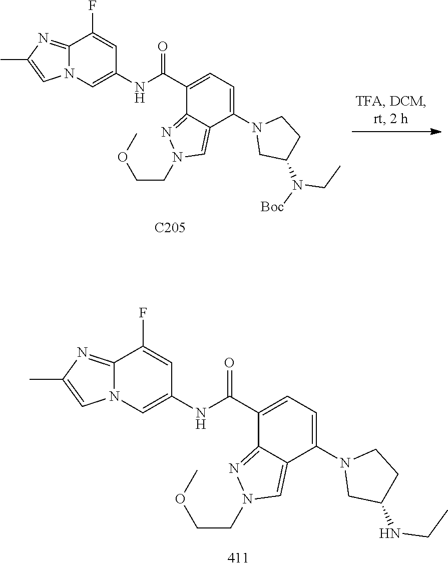

To a stirred mixture of tert-butyl N-ethyl-N-[(3S)-1-[7-({8-fluoro-2-methylimidazo[1,2-a]pyridin-6-yl}carbamoyl)-2-(2-methoxyethyl) indazol-4-yl]pyrrolidin-3-yl]carbamate (70 mg, 0.121 mmol, 1 equiv) in DCM (2 mL) was added TFA (0.4 mL) dropwise at room temperature. The resulting mixture was stirred for 2 hr at room temperature. The resulting mixture was concentrated under reduced pressure. The crude product was purified by Prep-HPLC (Condition 10, Gradient 2) to afford 4-[(3S)-3-(ethylamino)pyrrolidin-1-yl]-N-{8-fluoro-2-methylimidazo[1,2-a]pyridin-6-yl}-2-(2-methoxyethyl) indazole-7-carboxamide (Compound 411, 28.4 mg, 49%) as a solid. LCMS (ES, m/z): 422 [M+H]^{+ 1}H NMR (400 MHz, DMSO-d₆) δ 11.05 (s, 1H), 9.21 (d, J=1.7 Hz, 1H), 8.82 (s, 1H), 7.94 (d, J=8.3 Hz, 1H), 7.89 (d, J=3.0 Hz, 1H), 7.28 (dd, J=12.4, 1.7 Hz, 1H), 6.02 (d, J=8.4 Hz, 1H), 4.71 (t, J=5.2 Hz, 2H), 3.94 (t, J=5.2 Hz, 2H), 3.83-3.73 (m, 2H), 3.64 (d, J=8.2 Hz, 1H), 3.50-3.40 (m, 2H), 3.32 (s, 3H), 2.68-2.58 (m, 2H), 2.35 (s, 3H), 2.17 (dt, J=12.7, 6.1 Hz, 1H), 1.92 (dd, J=12.1, 6.4 Hz, 1H), 1.05 (t, J=7.1 Hz, 3H).

### Example 159: Synthesis of Compound 413

### Synthesis of Intermediate C206

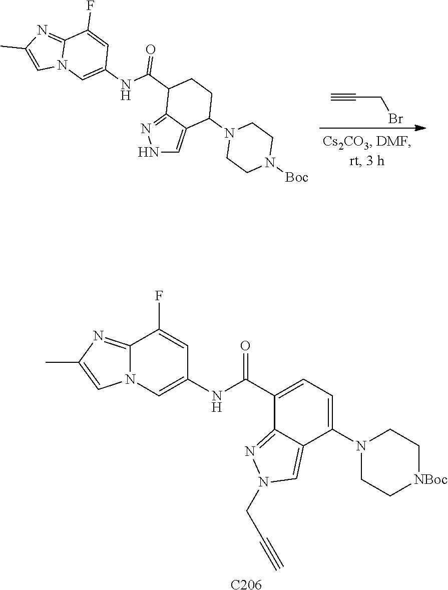

To a stirred mixture of tert-butyl 4-[7-({8-fluoro-2-methylimidazo[1,2-a]pyridin-6-yl}carbamoyl)-2H-indazol-4-yl]piperazine-1-carboxylate (100.0 mg, 0.20 mmol, 1.0 equiv) and 2-bromoacetonitrile (36.4 mg, 0.30 mmol, 1.5 equiv) in DMF (1 mL) was added Cs₂CO₃ (198.0 mg, 0.61 mmol, 3.0 equiv) at room temperature. The resulting mixture was stirred for 3 h at room temperature. The resulting mixture was diluted with water. The resulting mixture was extracted with ethyl acetate (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with EA to afford tert-butyl 4-[2-(cyanomethyl)-7-({8-fluoro-2-methylimidazo[1,2-a]pyridin-6-yl}carbamoyl) indazol-4-yl]piperazine-1-carboxylate (C206, 56 mg, 51%) as a solid. LCMS (ES, m/z): 533 [M+H]⁺ Synthesis of Compound 413

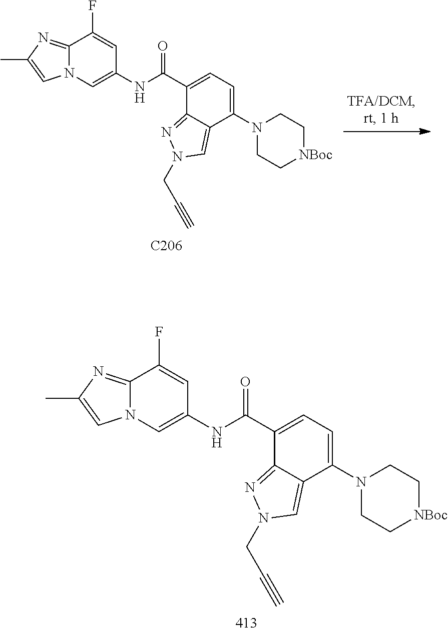 [SMILES: C#CCn1cc2c(N3CCN(C(=O)OC(C)(C)C)CC3)ccc(C(=O)Nc3cc(F)c4nc(C)cn4c3)c2n1 ; "C206" ; C#CCn1cc2c(N3CCN(C(=O)OC(C)(C)C)CC3)ccc(C(=O)Nc3cc(F)c4nc(C)cn4c3)c2n1 ; "413"]

A solution of tert-butyl 4-[2-(cyanomethyl)-7-({8-fluoro-2-methylimidazo[1,2-a]pyridin-6-yl}carbamoyl) indazol-4-yl]piperazine-1-carboxylate (56.0 mg, 0.10 mmol, 1.0 equiv) in DCM (0.5 mL) was treated with TFA (0.5 mL) at room temperature. The resulting mixture was stirred for 1 h at room temperature. The resulting mixture was concentrated under reduced pressure. The crude product was purified by Prep-HPLC (Condition 10, Gradient 6) to afford 2-(cyanomethyl)-N-{8-fluoro-2-methylimidazo[1,2-a]pyridin-6-yl}-4-(piperazin-1-yl) indazole-7-carboxamide (Compound 413, 6.1 mg, 13%) as a solid. LCMS (ES, m/z): 433 [M+H]^{+ 1}H NMR (300 MHz, DMSO-d₆) δ 10.94 (s, 1H), 9.26 (s, 1H), 9.01 (s, 1H), 8.03 (s, 1H), 7.92 (s, 1H), 7.27 (s, 1H), 6.54 (s, 1H), 5.94 (s, 2H), 3.33-3.32 (m, 4H), 2.92-2.94 (m, 4H), 2.35 (s, 3H).

### Example 160: Synthesis of Compound 414

### Synthesis of Intermediate C207

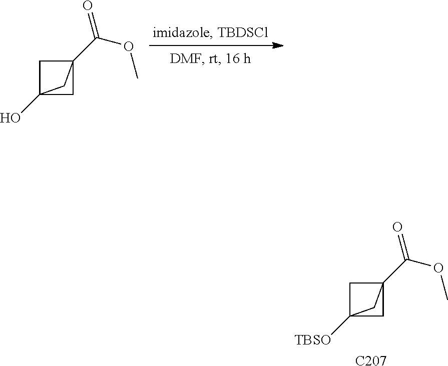

To a stirred mixture of methyl 3-hydroxybicyclo[1.1.1]pentane-1-carboxylate (500.0 mg, 3.51 mmol, 1.0 equiv) and imidazole (478.9 mg, 7.03 mmol, 2.0 equiv) in DMF (5 mL) was added TBSCl (636.1 mg, 4.22 mmol, 1.2 equiv) at room temperature. The resulting mixture was stirred for overnight at room temperature. The reaction was monitored by TLC. The resulting mixture was diluted with water. The resulting mixture was extracted with ethyl acetate (3×20 mL). The combined organic layers were washed with brine (1×10 mL), dried over anhydrous Na₂SO₄. After filtration, the filtrate was concentrated under reduced pressure. The crude product was used in the next step directly without further purification to afford methyl 3-[(tert-butyldimethylsilyl)oxy]bicyclo[1.1.1]pentane-1-carboxylate (C207, 900 mg, 99%) as a solid. ¹H NMR (400 MHz, Chloroform-d) δ 3.69 (s, 3H), 2.22 (s, 6H), 0.90 (s, 9H), 0.12 (s, 6H).

### Synthesis of Intermediate C208

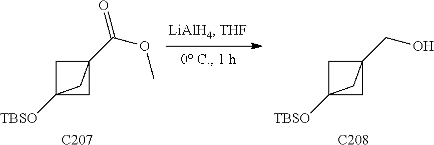 [SMILES: COC(=O)C12CC(O[Si](C)(C)C(C)(C)C)(C1)C2 ; CC(C)(C)[Si](C)(C)OC12CC(CO)(C1)C2 ; "C207" ; "C208"]

To a stirred solution of LiAlH₄ (266.4 mg, 7.02 mmol, 2.0 equiv) in THF (9 mL) was added methyl 3-[(tert-butyldimethylsilyl)oxy]bicyclo[1.1.1]pentane-1-carboxylate (900 mg, 3.51 mmol, 1.0 equiv) dropwise at 0° C. under nitrogen atmosphere. The resulting mixture was stirred for 1 hr at 0° C. under nitrogen atmosphere. The reaction was quenched with water (0.3 mL) and NaOH (15%) (0.3 mL) at 0° C. The resulting mixture was dried over anhydrous MgSO₄. After filtration, the filtrate was concentrated under reduced pressure. The crude product was used in the next step directly without further purification to afford {3-[(tert-butyldimethylsilyl)oxy]bicyclo[1.1.1]pentan-1-yl}methanol (C208, 700 mg, 87%) as a oil. ¹H NMR (400 MHz, Chloroform-d) δ 3.76 (s, 2H), 1.88 (s, 6H), 0.91 (s, 8H), 0.12 (s, 6H).

### Synthesis of Intermediate C209

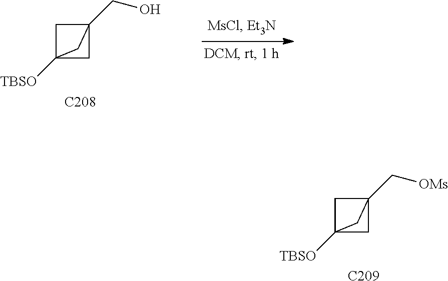 [SMILES: CC(C)(C)[Si](C)(C)OC12CC(CO)(C1)C2 ; "C208" ; CC(C)(C)[Si](C)(C)OC12CC(COS(C)(=O)=O)(C1)C2 ; "C209"]

To a stirred mixture of {3-[(tert-butyldimethylsilyl)oxy]bicyclo[1.1.1]pentan-1-yl}methanol (300 mg, 1.31 mmol, 1.0 equiv) and Et₃N (199.3 mg, 1.97 mmol, 1.5 equiv) in DCM (3 mL) was added MsCl (165.4 mg, 1.44 mmol, 1.1 equiv) dropwise at 0° C. under nitrogen atmosphere. The resulting mixture was stirred for 1 hr at room temperature under nitrogen atmosphere. The reaction was quenched with water. The resulting mixture was extracted with ethyl acetate (3×20 mL). The combined organic layers were washed with brine (1×10 mL), dried over anhydrous Na₂SO₄. After filtration, the filtrate was concentrated under reduced pressure to afford {3-[(tert-butyldimethylsilyl)oxy]bicyclo[1.1.1]pentan-1-yl}methyl methanesulfonate (C209, 235 mg, 58%) as a colorless oil without further purification.

### Synthesis of Intermediate C210

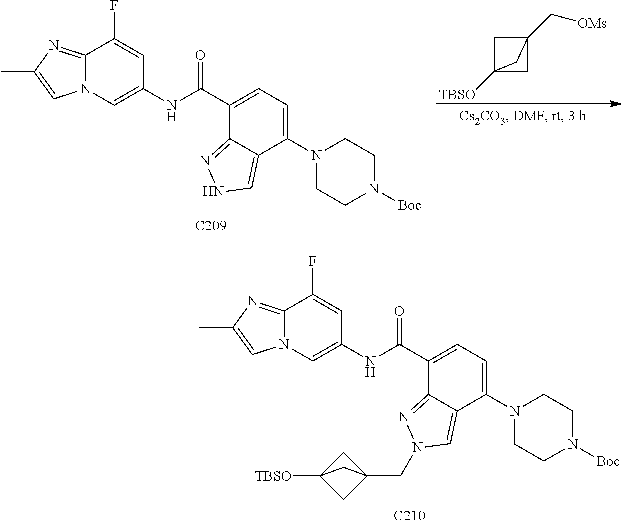 [SMILES: Cc1cn2cc(NC(=O)c3ccc(N4CCN(C(=O)OC(C)(C)C)CC4)c4c[nH]nc34)cc(F)c2n1 ; CC(C)(C)[Si](C)(C)OC12CC(COS(C)(=O)=O)(C1)C2 ; "C209" ; Cc1cn2cc(NC(=O)c3ccc(N4CCN(C(=O)OC(C)(C)C)CC4)c4cn(CC56CC(O[Si](C)(C)C(C)(C)C)(C5)C6)nc34)cc(F)c2n1 ; "C210"]

To a stirred mixture of tert-butyl 4-[7-({8-fluoro-2-methylimidazo[1,2-a]pyridin-6-yl}carbamoyl)-2H-indazol-4-yl]piperazine-1-carboxylate (235.0 mg, 0.476 mmol, 1.0 equiv) and {3-[(tert-butyldimethylsilyl)oxy]bicyclo[1.1.1]pentan-1-yl}methyl methanesulfonate (175.1 mg, 0.57 mmol, 1.2 equiv) in DMF (1.5 mL) was added Cs₂CO₃ (465.4 mg, 1.42 mmol, 3.0 equiv) at room temperature. The resulting mixture was stirred for 3 hr at room temperature. The resulting mixture was diluted with water. The resulting mixture was extracted with ethyl acetate (3×20 mL). The combined organic layers were washed with brine (1×10 mL), dried over anhydrous Na₂SO₄. After filtration, the filtrate was concentrated under reduced pressure. The residue was purified by silica gel column chromatography, eluted with EA to afford tert-butyl 4-[2-({3-[(tert-butyldimethylsilyl)oxy]bicyclo[1.1.1]pentan-1-yl}methyl)-7-({8-fluoro-2-methylimidazo[1,2-a]pyridin-6-yl}carbamoyl) indazol-4-yl]piperazine-1-carboxylate hydrofluoride (C210, 130 mg, 37%) as a solid. LCMS (ES, m/z): 704 [M+H]⁺

### Synthesis of Compound 414

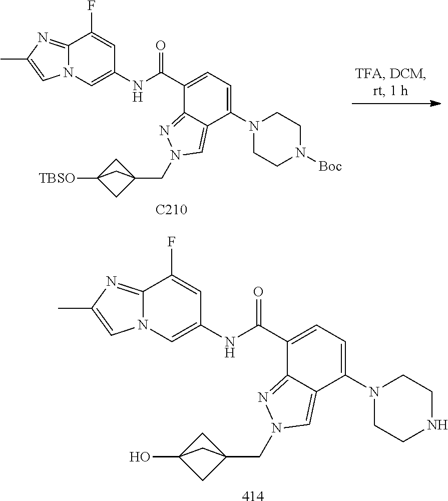 [SMILES: Cc1cn2cc(NC(=O)c3ccc(N4CCN(C(=O)OC(C)(C)C)CC4)c4cn(CC56CC(O[Si](C)(C)C(C)(C)C)(C5)C6)nc34)cc(F)c2n1 ; "C210" ; Cc1cn2cc(NC(=O)c3ccc(N4CCNCC4)c4cn(CC56CC(O)(C5)C6)nc34)cc(F)c2n1 ; "414"]

A solution of tert-butyl 4-[2-({3-[(tert-butyldimethylsilyl)oxy]bicyclo[1.1.1]pentan-1-yl}methyl)-7-({8-fluoro-2-methylimidazo[1,2-a]pyridin-6-yl}carbamoyl) indazol-4-yl]piperazine-1-carboxylate (130 mg, 0.18 mmol, 1.0 equiv) in DCM (1 mL) was treated with TFA (1 mL) at room temperature. The resulting mixture was stirred for 1 hr at room temperature. The resulting mixture was concentrated under reduced pressure. The crude product was purified by Prep-HPLC (Condition 14, Gradient 1) to afford N-{8-fluoro-2-methylimidazo[1,2-a]pyridin-6-yl}-2-({3-hydroxybicyclo[1.1.1]pentan-1-yl}methyl)-4-(piperazin-1-yl) indazole-7-carboxamide 2,2,2-trifluoroacetate (Compound 414, 52 mg, 46%) as a solid. LCMS (ES, m/z): 490 [M+H]^{+ 1}H NMR (400 MHz, DMSO-d₆) δ 11.35 (s, 1H), 9.48 (d, J=1.5 Hz, 1H), 8.97 (s, 2H), 8.89 (s, 1H), 8.18 (dd, J=2.6, 1.2 Hz, 1H), 8.05 (d, J=8.0 Hz, 1H), 7.66 (d, J=11.6 Hz, 1H), 6.63 (d, J=8.1 Hz, 1H), 4.79 (s, 2H), 3.61 (t, J=5.1 Hz, 4H), 3.37-3.35 (m 4H), 2.45 (d, J=1.0 Hz, 3H), 1.80 (s, 6H).

### Example 161: Synthesis of Compound 415

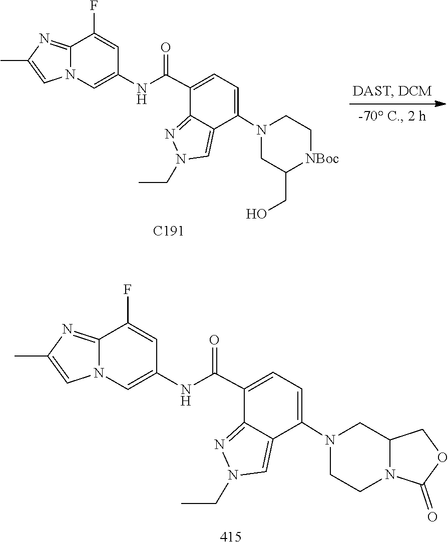

To a stirred solution of tert-butyl 4-[2-ethyl-7-({8-fluoro-2-methylimidazo[1,2-a]pyridin-6-yl}carbamoyl) indazol-4-yl]-2-(hydroxymethyl)piperazine-1-carboxylate (100 mg, 0.181 mmol, 1 equiv) in DCM (2 mL) was added DAST (60 mg, 0.372 mmol, 2.05 equiv) dropwise at −78° C. under nitrogen atmosphere. The resulting mixture was stirred for 2 hr at room temperature. The mixture was basified to pH 8 with NaHCO₃ aq. The resulting mixture was extracted with EA (3×5 mL). The combined organic layers were washed with NaCl (1×5 mL), dried over anhydrous Na₂SO₄. After filtration, the filtrate was concentrated under reduced pressure. The residue was purified by reverse flash chromatography (condition 3, Gradient 2) to afford 2-ethyl-N-{8-fluoro-2-methylimidazo[1,2-a]pyridin-6-yl}-4-{3-oxo-tetrahydro-1H-[1,3]oxazolo[3,4-a]pyrazin-7-yl}indazole-7-carboxamide (Compound 415, 20 mg, 23%) as a solid. LCMS (ES, m/z): 478 [M+H]^{+ 1}H NMR (300 MHz, DMSO-d₆) δ 11.10 (s, 1H), 9.22 (d, J=1.6 Hz, 1H), 8.88 (s, 1H), 8.00 (d, J=8.0 Hz, 1H), 7.91 (dd, J=3.2, 1.0 Hz, 1H), 7.32 (dd, J=12.4, 1.7 Hz, 1H), 6.59 (d, J=8.1 Hz, 1H), 4.61 (q, J=7.3 Hz, 2H), 4.47 (t, J=7.9 Hz, 1H), 4.20-4.01 (m, 3H), 3.89 (d, J=12.5 Hz, 1H), 3.79-3.68 (m, 1H), 3.02-2.82 (m, 2H), 2.36 (d, J=0.8 Hz, 3H), 1.64 (t, J=7.3 Hz, 3H).

### Example 162: Synthesis of Compound 416

### Synthesis of Intermediate 211

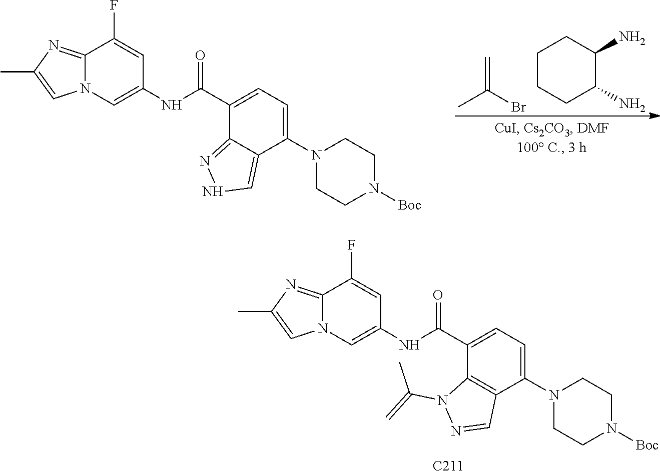

To a stirred mixture of tert-butyl 4-[7-({8-fluoro-2-methylimidazo[1,2-a]pyridin-6-yl}carbamoyl)-2H-indazol-4-yl]piperazine-1-carboxylate (200.0 mg, 0.40 mmol, 1.0 equiv) and 2-bromo-1-propene (73.5 mg, 0.60 mmol, 1.5 equiv) in DMF (4 mL) were added CS₂CO₃ (396.1 mg, 1.21 mmol, 3.0 equiv), (1R,2R)-cyclohexane-1,2-diamine (9.2 mg, 0.08 mmol, 0.2 equiv) and CuI (7.7 mg, 0.04 mmol, 0.1 equiv) at room temperature. The resulting mixture was stirred for 3 hr at 100° C. under nitrogen atmosphere. The mixture was allowed to cool down to room temperature. The resulting mixture was diluted with water and extracted with ethyl acetate (3×20 mL). The combined organic layers were washed with water (1×10 mL), brine (1×10 mL), dried over anhydrous Na₂SO₄. After filtration, the filtrate was concentrated under reduced pressure. The residue was purified by silica gel column chromatography, eluted with EA to afford tert-butyl 4-[7-({8-fluoro-2-methylimidazo[1,2-a]pyridin-6-yl}carbamoyl)-1-(prop-1-en-2-yl) indazol-4-yl]piperazine-1-carboxylate (C211, 108 mg, 49%) as a solid. LCMS (ES, m/z): 534 [M+H]⁺

### Synthesis of Compound 416

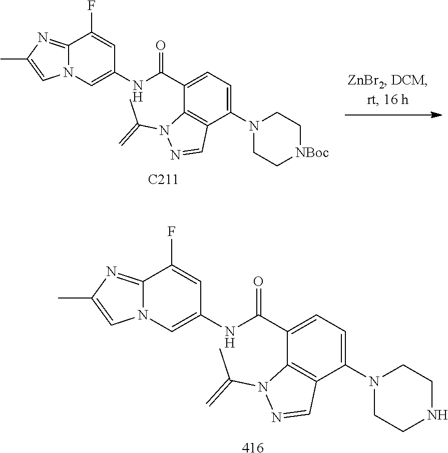 [SMILES: C=C(C)n1ncc2c(N3CCN(C(=O)OC(C)(C)C)CC3)ccc(C(=O)Nc3cc(F)c4nc(C)cn4c3)c21 ; "C211" ; C=C(C)n1ncc2c(N3CCNCC3)ccc(C(=O)Nc3cc(F)c4nc(C)cn4c3)c21 ; "416"]

To a stirred mixture of tert-butyl 4-(7-((8-fluoro-2-methylimidazo[1,2-a]pyridin-6-yl)carbamoyl)-1-(prop-1-en-2-yl)-1H-indazol-4-yl)piperazine-1-carboxylate (65.0 mg, 0.122 mmol, 1.0 equiv) in DCM (2 mL) were added ZnBr₂ (270.7 mg, 1.220 mmol, 10.0 equiv) at room temperature. The resulting mixture was stirred for 16 hr at room temperature. The resulting mixture was concentrated under reduced pressure. The crude product was purified by Prep-HPLC (Condition 10, Gradient 7) to afford N-(8-fluoro-2-methylimidazo[1,2-a]pyridin-6-yl)-4-(piperazin-1-yl)-1-(prop-1-en-2-yl)-1H-indazole-7-carboxamide (Compound 416, 27.4 mg, 51%) as a solid. LCMS (ES, m/z): 434 [M+H]^{+ 1}H NMR (400 MHz, DMSO-d₆) δ 10.42 (s, 1H), 9.08 (d, J=1.6 Hz, 1H), 8.35 (s, 1H), 7.95 (d, J=3.0 Hz, 1H), 7.49 (d, J=7.9 Hz, 1H), 7.14 (dd, J=12.6, 1.6 Hz, 1H), 6.61 (d, J=7.9 Hz, 1H), 5.01 (d, J=1.6 Hz, 1H), 4.77 (s, 1H), 3.27 (t, J=4.9 Hz, 1H), 2.94 (t, J=4.8 Hz, 4H), 2.34 (s, 3H), 2.23 (s, 3H).

### Example 163: Synthesis of Compound 417

### Synthesis of Intermediate C212

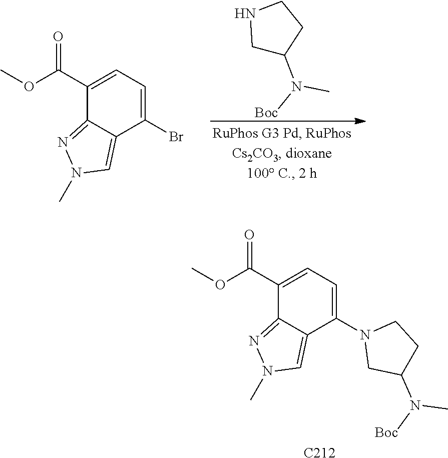

To a stirred mixture of methyl 4-bromo-2-methylindazole-7-carboxylate (1.5 g, 5.574 mmol, 1 equiv) and tert-butyl N-methyl-N-(pyrrolidin-3-yl)carbamate (1.34 g, 6.689 mmol, 1.2 equiv) in dioxane (30 mL) were added Cs₂CO₃ (5.45 g, 16.722 mmol, 3 equiv) and RuPhos (0.52 g, 1.115 mmol, 0.2 equiv) and RuPhos Palladacycle Gen.3 (0.47 g, 0.557 mmol, 0.1 equiv) at room temperature under nitrogen atmosphere. The resulting mixture was stirred for 2 hr at 100° C. under nitrogen atmosphere. The resulting mixture was concentrated under reduced pressure. The residue was purified by silica gel column chromatography, eluted with CH₂Cl₂/MeOH (10:1) to afford methyl 4-{3-[(tert-butoxycarbonyl)(methyl)amino]pyrrolidin-1-yl}-2-methylindazole-7-carboxylate (C212, 2.45 g, 113%) as a solid. LCMS (ES, m/z): 389 [M+H]⁺

### Synthesis of Intermediate C213

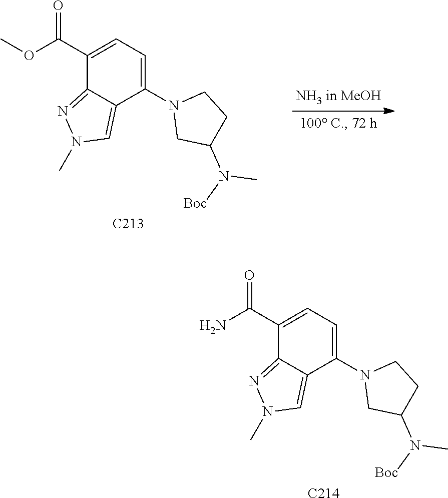

To a stirred mixture of methyl 4-{3-[(tert-butoxycarbonyl)(methyl)amino]pyrrolidin-1-yl}-2-methylindazole-7-carboxylate (2.4 g, 6.178 mmol, 1 equiv) in NH₃(g) (7 M in MeOH) (200 mL) at room temperature. The resulting mixture was stirred for 72 hr at sealed 100° C. under NH₃ atmosphere. The mixture was allowed to cool down to room temperature. The resulting mixture was concentrated under vacuum. The residue was purified by silica gel column chromatography, eluted with EA to afford tert-butyl N-[1-(7-carbamoyl-2-methylindazol-4-yl)pyrrolidin-3-yl]-N-methylcarbamate (C214, 1.87 g, 62%) as a solid.

LCMS (ES, m/z): 374 [M+H]⁺

### Synthesis of Intermediate C215

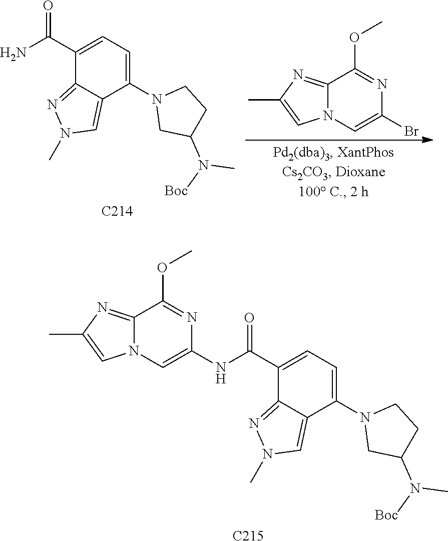

To a stirred mixture of tert-butyl N-[1-(7-carbamoyl-2-methylindazol-4-yl)pyrrolidin-3-yl]-N-methylcarbamate (280.0 mg, 0.577 mmol, 1 equiv) and 6-bromo-8-methoxy-2-methylimidazo[1,2-a]pyrazine (209.6 mg, 0.865 mmol, 1.5 equiv) in Dioxane (5.39 mL) were added Cs₂CO₃ (564.3 mg, 1.731 mmol, 3.0 equiv) and XantPhos (66.8 mg, 0.115 mmol, 0.2 equiv) and Pd₂(dba)₃ (52.9 mg, 0.058 mmol, 0.1 equiv) at room temperature under nitrogen atmosphere. The resulting mixture was stirred for 2 hr at 100° C. under nitrogen atmosphere. The mixture was allowed to cool down to room temperature. The resulting mixture was diluted with water (5 mL). The resulting mixture was extracted with ethyl acetate (3×5 mL). The combined organic layers were washed with brine (lx 5 mL), dried over anhydrous Na₂SO₄. After filtration, the filtrate was concentrated under reduced pressure. The residue was purified by silica gel column chromatography, eluted with EA to afford tert-butyl N-{1-[7-({8-methoxy-2-methylimidazo[1,2-a]pyrazin-6-yl}carbamoyl)-2-methylindazol-4-yl]pyrrolidin-3-yl}-N-methylcarbamate (C215, 170 mg, 55%) as a solid. LCMS (ES, m/z): 535 [M+H]⁺

### Synthesis of Compound 417

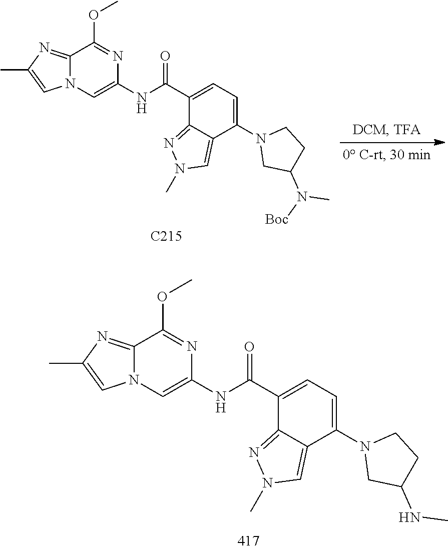

To a stirred mixture of tert-butyl N-[1-(7-{[8-(dimethylamino)-2-methylimidazo[1,2-a]pyrazin-6-yl]carbamoyl}-2-methylindazol-4-yl)pyrrolidin-3-yl]-N-methylcarbamate (160 mg, 0.292 mmol, 1 equiv) in DCM (3 mL) was added TFA (0.6 mL) dropwise at 0° C. The resulting mixture was stirred for 30 min at room temperature. The resulting mixture was concentrated under vacuum. The crude product was purified by Prep-HPLC (Condition 10, Gradient 8) to afford N-[8-(dimethylamino)-2-methylimidazo[1,2-a]pyrazin-6-yl]-2-methyl-4-[3-(methylamino)pyrrolidin-1-yl]indazole-7-carboxamide (85 mg, 65%) as a solid. LCMS (ES, m/z): 435 [M+H]^{+ 1}H NMR (400 MHz, DMSO-d₆) δ 11.20 (s, 1H), 8.99 (s, 1H), 8.83 (s, 1H), 7.95 (d, J=8.3 Hz, 1H), 7.90 (d, J=1.0 Hz, 1H), 6.02 (d, J=8.5 Hz, 1H), 4.24 (s, 3H), 4.10 (s, 3H), 3.80-3.73 (m, 2H), 3.69 (d, J=27.8 Hz, 1H), 3.43 (dd, J=10.2, 4.0 Hz, 1H), 3.32 (s, 1H), 2.34 (d, J=2.8 Hz, 6H), 2.15 (dp, J=13.1, 7.2, 6.5 Hz, 1H), 1.92 (dd, J=12.0, 6.5 Hz, 1H).

### Example 164: Synthesis of Compounds 418, 431, and 432

### Synthesis of Intermediate C216

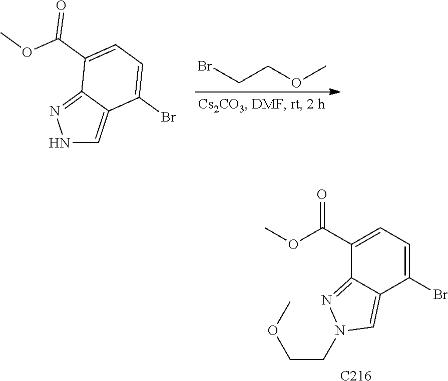

To a stirred solution/mixture of methyl 4-bromo-2H-indazole-7-carboxylate (3.0 g, 11.761 mmol, 1.0 equiv) and Cs₂CO₃ (7.6 g, 23.522 mmol, 2.0 equiv) in dimethylformamide (50 mL) were added 2-bromoethyl methyl ether (2.45 g, 17.642 mmol, 1.5 equiv) dropwise at room temperature. The resulting mixture was stirred for 2 hr at room temperature. The resulting mixture was diluted with water (50 mL). The resulting mixture was extracted with ethyl acetate (2×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (1:1) to afford methyl 4-bromo-2-(2-methoxyethyl) indazole-7-carboxylate (1.2 g, 29%) as a solid. LCMS (ES, m/z): 313 [M+H]⁺

### Synthesis of Intermediate C217

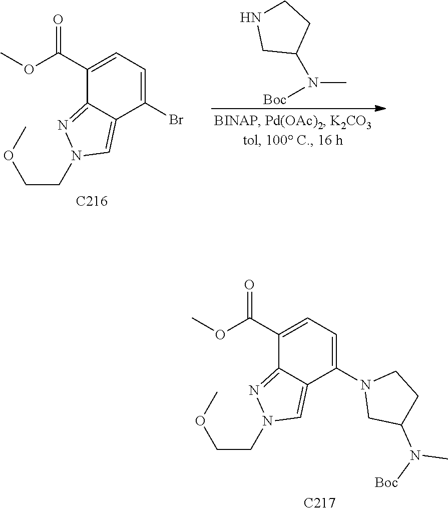

To a solution of methyl 4-bromo-2-(2-methoxyethyl) indazole-7-carboxylate (1.1 g, 3.513 mmol, 1.0 equiv) and tert-butyl N-methyl-N-(pyrrolidin-3-yl)carbamate (1.4 g, 7.026 mmol, 2.0 equiv) in toluene (20 mL) were added potassium methaneperoxoate potassium (0.9 g, 7.026 mmol, 2.0 equiv) and BINAP (0.4 g, 0.703 mmol, 0.2 equiv), Pd(OAc)₂ (0.08 g, 0.351 mmol, 0.1 equiv). After stirring for 16 hr at 100° C. under a nitrogen atmosphere, the resulting mixture was concentrated under reduced pressure. The residue was purified by silica gel column chromatography, eluted with CH₂Cl₂/MeOH (30:1) to afford methyl 4-{3-[(tert-butoxycarbonyl) (methyl)amino]pyrrolidin-1-yl}-2-(2-methoxyethyl) indazole-7-carboxylate (C217, 1.5 g, 88%) as an oil. LCMS (ES, m/z): 433 [M+H]⁺

### Synthesis of Intermediate C218

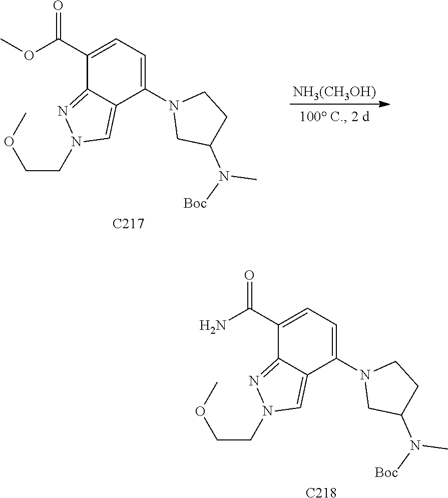

To a solution of methyl 4-{3-[(tert-butoxycarbonyl)(methyl)amino]pyrrolidin-1-yl}-2-(2-methoxyethyl) indazole-7-carboxylate (500.0 mg, 1.156 mmol, 1.0 equiv) was added NH₃(g) in methanol (50 mL) in a pressure tank. The resulting mixture was stirred for 2 days at 100° C. The resulting mixture was concentrated under reduced pressure. The residue was purified by silica gel column chromatography, eluted with CH₂Cl₂/MeOH (30:1) to afford tert-butyl N-{1-[7-carbamoyl-2-(2-methoxyethyl) indazol-4-yl]pyrrolidin-3-yl}-N-methylcarbamate (C218, 450 mg, 74%) as a solid. LCMS (ES, m/z): 418 [M+H]⁺

### Synthesis of Intermediate C219

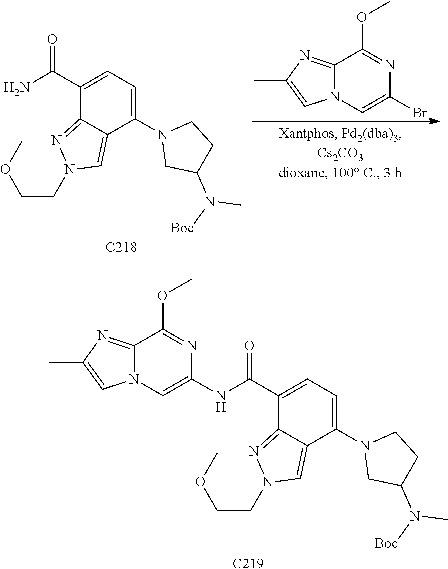

To a solution of tert-butyl N-{1-[7-carbamoyl-2-(2-methoxyethyl) indazol-4-yl]pyrrolidin-3-yl}-N-methylcarbamate (200.0 mg, 0.479 mmol, 1.0 equiv) and 6-bromo-8-methoxy-2-methylimidazo[1,2-a]pyrazine (150.7 mg, 0.623 mmol, 1.3 equiv) in dioxane (5 mL) were added caesio methaneperoxoate caesium (391.4 mg, 1.198 mmol, 2.5 equiv) and Pd₂(dba)₃ (43.8 mg, 0.048 mmol, 0.1 equiv), Xantphos (55.4 mg, 0.096 mmol, 0.2 equiv). After stirring for 3 hr at 100° C. under a nitrogen atmosphere, the resulting mixture was concentrated under reduced pressure. The residue was purified by silica gel column chromatography, eluted with CH₂Cl₂/MeOH (40:1) to afford tert-butyl N-{1-[7-({8-methoxy-2-methylimidazo[1,2-a]pyrazin-6-yl}carbamoyl)-2-(2-methoxyethyl) indazol-4-yl]pyrrolidin-3-yl}-N-methylcarbamate (C219, 200 mg, 57%) as a solid. LCMS (ES, m/z): 579 [M+H]⁺

### Synthesis of Compound 418

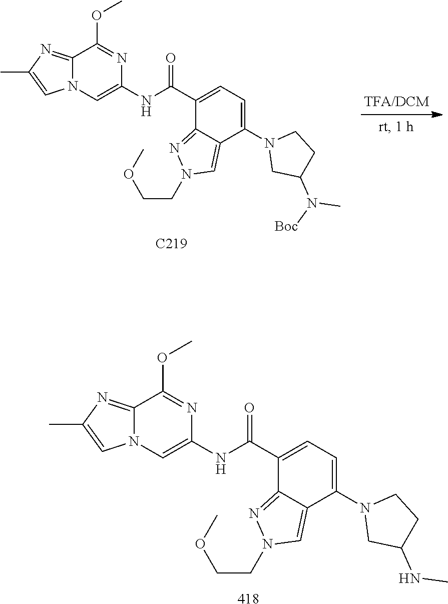

To a stirred solution/mixture of tert-butyl N-{1-[7-({8-methoxy-2-methylimidazo[1,2-a]pyrazin-6-yl}carbamoyl)-2-(2-methoxyethyl) indazol-4-yl]pyrrolidin-3-yl}-N-methylcarbamate (180.0 mg, 0.311 mmol, 1.0 equiv) in DCM (2 mL) were added TFA (1 mL) dropwise at room temperature. The resulting mixture was stirred for 1 hr at room temperature. The resulting mixture was concentrated under reduced pressure. The crude product was purified by Prep-HPLC (Condition 3, Gradient 1) to afford N-{8-methoxy-2-methylimidazo[1,2-a]pyrazin-6-yl}-2-(2-methoxyethyl)-4-[3-(methylamino)pyrrolidin-1-yl]indazole-7-carboxamide (Compound 418, 50 mg, 32%) as a solid. LCMS (ES, m/z): 479 [M+H]^{+ 1}H NMR (400 MHz, DMSO-d₆) δ 11.33 (s, 1H), 8.98 (s, 1H), 8.86 (s, 1H), 7.96 (d, J=8.3 Hz, 1H), 7.91 (d, J=1.0 Hz, 1H), 6.04 (d, J=8.4 Hz, 1H), 4.66 (t, J=5.3 Hz, 2H), 4.10 (s, 3H), 4.03 (t, J=5.3 Hz, 2H), 3.81-3.74 (m, 2H), 3.67-3.64 (m, 1H), 3.50-3.40 (m, 2H), 3.29 (s, 3H), 2.36 (d, J=19.5 Hz, 6H), 2.24-2.14 (m, 1H), 1.97-1.95 (m, 1H).

### Synthesis of Compound 431

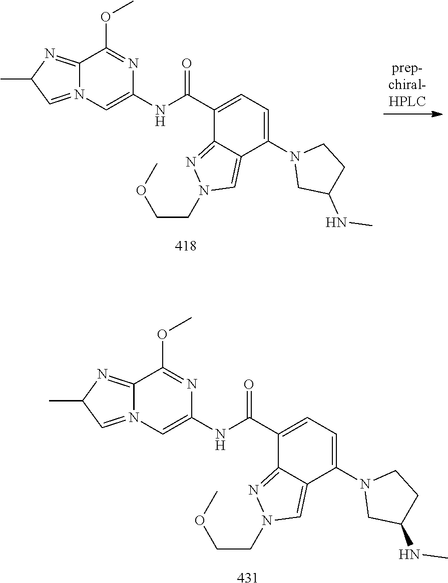

Compound 418 was separated by prep-chiral-HPLC (Condition 2, Gradient 2) to yield (R)—N-(8-methoxy-2-methylimidazo[1,2-a]pyrazin-6-yl)-2-(2-methoxyethyl)-4-(3-(methylamino)pyrrolidin-1-yl)-2H-indazole-7-carboxamide (Compound 431, 5.7 mg, 13%) as a solid. LCMS (ES, m/z): 479 [M+H]^{+ 1}H NMR (400 MHz, DMSO-d₆) δ 11.33 (s, 1H), 8.98 (s, 1H), 8.86 (s, 1H), 7.96 (d, J=8.3 Hz, 1H), 7.91 (d, J=1.0 Hz, 1H), 6.04 (d, J=8.4 Hz, 1H), 4.66 (t, J=5.3 Hz, 2H), 4.10 (s, 3H), 4.03 (t, J=5.3 Hz, 2H), 3.80-3.75 (m, 2H), 3.67-3.65 (m, 1H), 3.50-3.40 (m, 2H), 3.29 (s, 3H), 2.36 (d, J=19.5 Hz, 6H), 2.24-2.14 (m, 1H), 1.97-1.88 (m, 1H).

### Synthesis of Compound 432

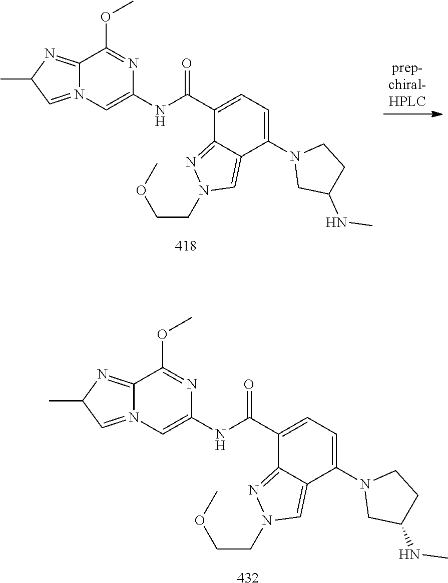

Compound 418 was separated by prep-chiral-HPLC (Condition 2, Gradient 2) to yield (S)—N-(8-methoxy-2-methylimidazo[1,2-a]pyrazin-6-yl)-2-(2-methoxyethyl)-4-(3-(methylamino)pyrrolidin-1-yl)-2H-indazole-7-carboxamide (Compound 432, 6.7 mg, 15%) as a solid. LCMS (ES, m/z): 479 [M+H]^{+ 1}H NMR (400 MHz, DMSO-d₆) δ 11.33 (s, 1H), 8.98 (s, 1H), 8.86 (s, 1H), 7.96 (d, J=8.3 Hz, 1H), 7.91 (d, J=1.0 Hz, 1H), 6.04 (d, J=8.4 Hz, 1H), 4.66 (t, J=5.3 Hz, 2H), 4.10 (s, 3H), 4.03 (t, J=5.3 Hz, 2H), 3.84-3.78 (m, 2H), 3.67-3.65 (m, 1H), 3.50-3.40 (m, 2H), 3.29 (s, 3H), 2.36 (d, J=19.5 Hz, 6H), 2.24-2.14 (m, 1H), 1.97-1.95 (m, 1H).

### Example 165: Synthesis of Compound 419

### Synthesis of Intermediate C220

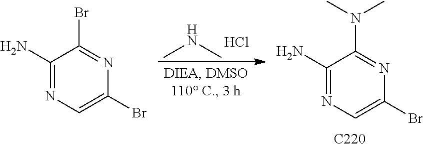

To a stirred mixture of 3,5-dibromopyrazin-2-amine (10.00 g, 39.542 mmol, 1 equiv) and Dimethylamine hydrochloride (3.55 g, 43.496 mmol, 1.1 equiv) in DMSO (100 mL) was added DIEA (15.33 g, 118.626 mmol, 3.0 equiv) at room temperature. The resulting mixture was stirred for 3 h at 110° C. The mixture was allowed to cool down to room temperature. The resulting mixture was diluted with water (200 mL). The resulting mixture was extracted with ethyl acetate (3×200 mL). The combined organic layers were washed with water (2×200 mL), dried over anhydrous Na₂SO₄. After filtration, the filtrate was concentrated under reduced pressure. The residue was purified by silica gel column chromatography, eluted with PE/EA (3:1) to afford 6-bromo-N2, N2-dimethylpyrazine-2,3-diamine (C220, 6.5 g, 75%) as a solid. LCMS (ES, m/z): 217 [M+H]⁺

### Synthesis of Intermediate C221

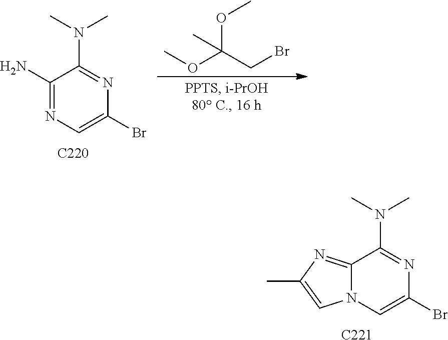

To a stirred mixture of 6-bromo-N2,N2-dimethylpyrazine-2,3-diamine (6.50 g, 29.944 mmol, 1 equiv) and 1-bromo-1,1-dimethoxyethane (6.07 g, 35.933 mmol, 1.2 equiv) in i-PrOH (130 mL) was added PPTS (0.75 g, 2.994 mmol, 0.1 equiv) in portions at room temperature. The resulting mixture was stirred for 16 hr at 80° C. The mixture was allowed to cool down to room temperature. The resulting mixture was filtered and the filter cake was washed with i-PrOH (3×100 mL). The filter cake was dried to afford 6-bromo-N,N,2-trimethylimidazo[1,2-a]pyrazin-8-amine (C221, 5.1 g, 66%) as a solid. LCMS (ES, m/z): 255 [M+H]⁺

### Synthesis of Intermediate C222

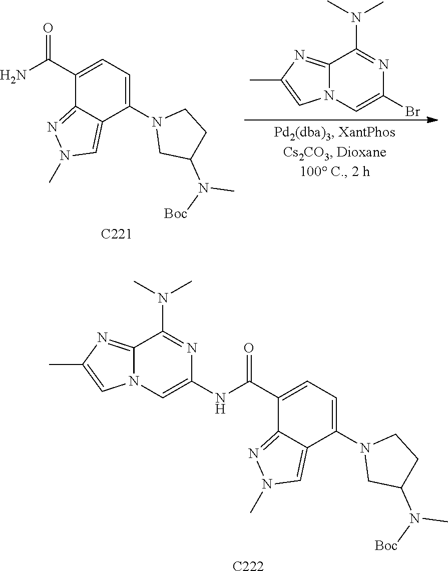

To a stirred mixture of tert-butyl N-[1-(7-carbamoyl-2-methylindazol-4-yl)pyrrolidin-3-yl]-N-methylcarbamate (280.0 mg, 0.577 mmol, 1.0 equiv) and 6-bromo-N,N,2-trimethylimidazo[1,2-a]pyrazin-8-amine (220.9 mg, 0.865 mmol, 1.5 equiv) in dioxane (6 mL) were added Cs₂CO₃ (564.3 mg, 1.731 mmol, 3 equiv) and XantPhos (66.8 mg, 0.115 mmol, 0.2 equiv) and Pd₂(dba)₃ (52.9 mg, 0.058 mmol, 0.1 equiv) at room temperature under nitrogen atmosphere. The resulting mixture was stirred for 2 hr at 100° C. under nitrogen atmosphere. The mixture was allowed to cool down to room temperature. The resulting mixture was diluted with water (5 mL). The resulting mixture was extracted with ethyl acetate (3×5 mL). The combined organic layers were washed with brine (1×5 mL), dried over anhydrous Na₂SO₄. After filtration, the filtrate was concentrated under reduced pressure. The residue was purified by silica gel column chromatography, eluted with EA to afford tert-butyl N-[1-(7-{[8-(dimethylamino)-2-methylimidazo[1,2-a]pyrazin-6-yl]carbamoyl}-2-methylindazol-4-yl)pyrrolidin-3-yl]-N-methylcarbamate (C222, 170 mg, 53%) as a solid. LCMS (ES, m/z): 548 [M+H]⁺

### Synthesis of Compound 419

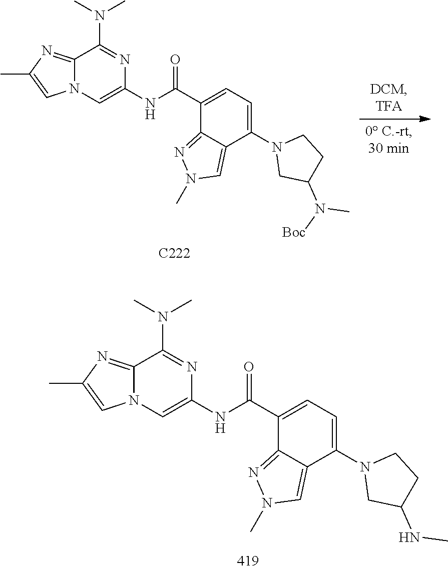

To a stirred mixture of tert-butyl N-[1-(7-{[8-(dimethylamino)-2-methylimidazo[1,2-a]pyrazin-6-yl]carbamoyl}-2-methylindazol-4-yl)pyrrolidin-3-yl]-N-methylcarbamate (160 mg, 0.292 mmol, 1 equiv) in DCM (3 mL) was added TFA (0.6 mL) dropwise at 0° C. The resulting mixture was stirred for 30 min at room temperature. The resulting mixture was concentrated under vacuum. The crude product was purified by Prep-HPLC (Condition 10, Gradient 5) to afford N-[8-(dimethylamino)-2-methylimidazo[1,2-a]pyrazin-6-yl]-2-methyl-4-[3-(methylamino)pyrrolidin-1-yl]indazole-7-carboxamide (Compound 419, 85 mg, 65%) as a solid. LCMS (ES, m/z): 448 [M+H]^{+ 1}H NMR (400 MHz, DMSO-d₆) δ 11.06 (s, 1H), 8.81 (s, 1H), 8.62 (s, 1H), 7.92 (d, J=8.3 Hz, 1H), 7.73 (d, J=1.0 Hz, 1H), 6.00 (d, J=8.4 Hz, 1H), 4.22 (s, 3H), 3.75 (dq, J=22.4, 7.9, 6.9 Hz, 2H), 3.64 (q, J=8.4, 7.8 Hz, 1H), 3.53 (s, 6H), 3.42 (dd, J=9.9, 4.1 Hz, 1H), 3.35 (d, J=5.5 Hz, 1H), 2.33 (d, J=10.7 Hz, 6H), 2.14 (dt, J=13.0, 6.5 Hz, 1H), 1.92 (dt, J=11.6, 5.8 Hz, 1H).

### Example 166: Synthesis of Compound 421

### Synthesis of Intermediate C223

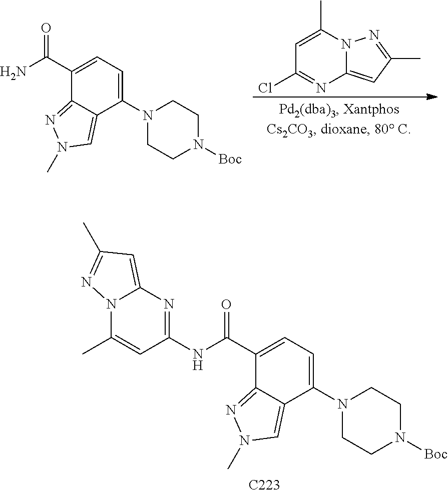 [SMILES: Cn1cc2c(N3CCN(C(=O)OC(C)(C)C)CC3)ccc(C(N)=O)c2n1 ; Cc1cc2nc(Cl)cc(C)n2n1 ; Cc1cc2nc(NC(=O)c3ccc(N4CCN(C(=O)OC(C)(C)C)CC4)c4cn(C)nc34)cc(C)n2n1 ; "C223"]

A solution of tert-butyl 4-(7-carbamoyl-2-methylindazol-4-yl)piperazine-1-carboxylate (60 mg, 0.167 mmol, 1 equiv), 5-chloro-2,7-dimethylpyrazolo[1,5-a]pyrimidine (36.38 mg, 0.200 mmol, 1.2 equiv), Pd₂(dba)₃ (15.29 mg, 0.017 mmol, 0.1 equiv), XantPhos (19.32 mg, 0.033 mmol, 0.2 equiv) and Cs₂CO₃ (163.17 mg, 0.501 mmol, 3 equiv) in dioxane (2 mL) was stirred for 4 h at 80° C. under N₂ atmosphere. The resulting mixture was extracted with DCM and water. The combined organic layers were dried over anhydrous Na₂SO₄. After filtration, the filtrate was concentrated under reduced pressure. The residue was purified by silica gel column chromatography, eluted with PE:EA=5:1 to afford tert-butyl 4-[7-({2,7-dimethylpyrazolo[1,5-a]pyrimidin-5-yl}carbamoyl)-2-methylindazol-4-yl]piperazine-1-carboxylate (C223, 47 mg, 55%) as a solid. LCMS (ES, m/z): 505 [M+H]⁺

### Synthesis of Compound 421

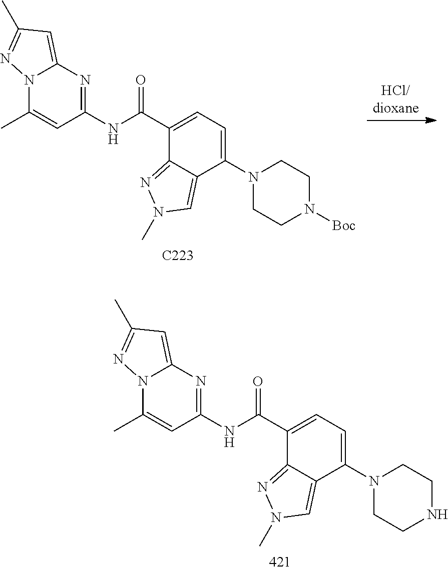

A solution of tert-butyl 4-[7-({2,7-dimethylpyrazolo[1,5-a]pyrimidin-5-yl}carbamoyl)-2-methylindazol-4-yl]piperazine-1-carboxylate (47 mg, 0.093 mmol, 1 equiv) in HCl (gas) in 1,4-dioxane (1 mL) for 1 hr at room temperature. The resulting mixture was concentrated under reduced pressure. The residue was purified by Prep-TLC (Condition 1, Gradient 2) to afford N-{2,7-dimethylpyrazolo[1,5-a]pyrimidin-5-yl}-2-methyl-4-(piperazin-1-yl) indazole-7-carboxamide (C224, 18 mg, 47%) as a solid. LCMS (ES, m/z): 405 [M+H]⁺1H NMR (300 MHz, Methanol-d4) δ 8.54 (s, 1H), 8.13 (d, J=8.2 Hz, 1H), 7.78 (s, 1H), 6.54 (d, J=8.2 Hz, 1H), 6.29 (s, 1H), 4.38 (s, 3H), 3.58-3.49 (m, 4H), 3.09 (t, J=5.0 Hz, 4H), 2.57 (d, J=6.3 Hz, 6H).

### Example 167: Synthesis of Compound 422

### Synthesis of Intermediate C224

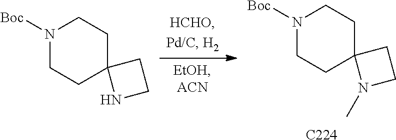 [SMILES: CC(C)(C)OC(=O)N1CCC2(CCN2)CC1 ; CN1CCC12CCN(C(=O)OC(C)(C)C)CC2 ; "C224"]

To a mixture of tert-butyl 1,7-diazaspiro[3.5]nonane-7-carboxylate (210 mg, 0.928 mmol, 1 equiv) and HCHO (55.72 mg, 1.856 mmol, 2 equiv) in EtOH/MeCN (2:1) (5 ml) was added Pd/C (100 mg, 0.094 mmol, 0.10 equiv, 10%) under nitrogen atmosphere in a 50 mL round-bottom flask. The mixture was hydrogenated at room temperature overnight under hydrogen atmosphere using a hydrogen balloon, filtered through filter paper and concentrated under reduced pressure to afford tert-butyl 1-methyl-1,7-diazaspiro[3.5]nonane-7-carboxylate (C224, 65 mg, 29%) as an oil. LCMS (ES, m/z): 241 [M+H]⁺

### Synthesis of Intermediate C225

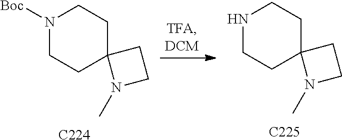

To a stirred solution of tert-butyl 1-methyl-1,7-diazaspiro[3.5]nonane-7-carboxylate (35 mg, 0.146 mmol, 1 equiv) in DCM (0.5 mL) was added TFA (0.5 mL, 6.732 mmol, 46.23 equiv) at room temperature. The resulting mixture was stirred for 2 hr at room temperature. The resulting mixture was concentrated under reduced pressure. The crude product was used in the next step directly without further purification. LCMS (ES, m/z): 141 [M+H]⁺

### Synthesis of Compound 422

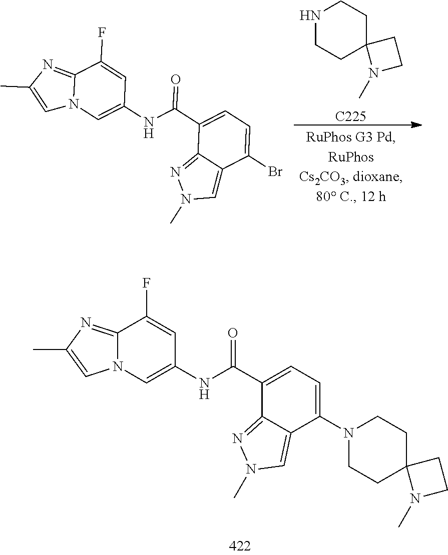

To a stirred mixture of 4-bromo-N-{8-fluoro-2-methylimidazo[1,2-a]pyridin-6-yl}-2-methylindazole-7-carboxamide (65 mg, 0.162 mmol, 1 equiv) and Intermediate C225 (27.19 mg, 0.194 mmol, 1.2 equiv) in dioxane (1 mL) were added Cs₂CO₃ (157.96 mg, 0.486 mmol, 3 equiv), RuPhos (15.08 mg, 0.032 mmol, 0.2 equiv) and RuPhos Palladacycle Gen.3 (13.52 mg, 0.016 mmol, 0.1 equiv) at room temperature under nitrogen atmosphere. The resulting mixture was stirred for 12 h at 80° C. under nitrogen atmosphere. The resulting mixture was concentrated under reduced pressure. The crude product was purified by Prep-HPLC (Condition 15, Gradient 1) to afford N-{8-fluoro-2-methylimidazo[1,2-a]pyridin-6-yl}-2-methyl-4-{1-methyl-1,7-diazaspiro[3.5]nonan-7-yl}indazole-7-carboxamide; bis(trifluoroacetic acid) (Compound 422, 1.9 mg, 10%) as a solid. LCMS (ES, m/z): 462 [M+H]⁺1H NMR (400 MHz, Methanol-d₄) δ 9.47 (d, J=1.5 Hz, 1H), 8.59 (s, 1H), 8.14 (d, J=8.1 Hz, 1H), 8.07-8.02 (m, 1H), 7.92 (dd, J=11.5, 1.5 Hz, 1H), 6.63 (d, J=8.1 Hz, 1H), 4.37 (s, 3H), 4.28 (d, J=8.7 Hz, 1H), 4.09 (d, J=12.7 Hz, 1H), 3.98 (t, J=14.2 Hz, 2H), 3.23-3.12 (m, 2H), 2.86 (s, 3H), 2.61 (t, J=8.2 Hz, 2H), 2.57 (d, J=1.0 Hz, 3H), 2.44 (d, J=12.3 Hz, 1H), 2.31 (d, J=13.1 Hz, 3H).

### Example 168: Synthesis of Compound 427

### Synthesis of Intermediate C226

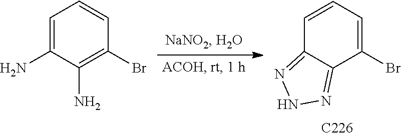 [SMILES: Nc1cccc(Br)c1N ; Brc1cccc2n[nH]nc12 ; "C226"]

To a stirred solution of 3-bromobenzene-1,2-diamine (4.0 g, 21.386 mmol, 1.0 equiv) in acetic acid (20 mL) and water (20 mL) were added sodium nitrite (1.6 g, 23.525 mmol, 1.1 equiv) in portions at room temperature. The resulting mixture was stirred for 1 hr at room temperature. The precipitated solids were collected by filtration and washed with water (2×20 mL). The solid was dried and this resulted in 4-bromo-2H-1,2,3-benzotriazole (C226, 3.2 g, 71%) as a solid. LCMS (ES, m/z): 198 [M+H]⁺

### Synthesis of Intermediate C227

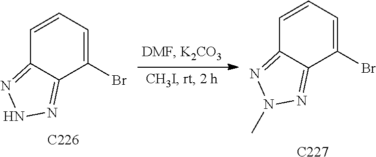

To a stirred mixture of 4-bromo-2H-1,2,3-benzotriazole (2.7 g, 13.635 mmol, 1.0 equiv) and K₂CO₃ (3.76 g, 27.270 mmol, 2.0 equiv) in dimethylformamide (60 mL) were added methyl iodide (2.9 g, 20.453 mmol, 1.5 equiv) dropwise at 0° C. The resulting mixture was stirred for 1 hr at room temperature. The resulting mixture was diluted with water (200 mL). The resulting mixture was extracted with ethyl acetate (2×100 mL). The combined organic layers were washed with water (2×200 mL), brine (1×200 mL), dried over anhydrous Na₂SO₄. After filtration, the filtrate was concentrated under reduced pressure. The residue was purified by silica gel column chromatography, eluted with CH₂Cl₂/PE (5:1) to afford 4-bromo-2-methyl-1,2,3-benzotriazole (C227, 0.9 g, 28%) as a solid. LCMS (ES, m/z): 212 [M+H]⁺

### Synthesis of Intermediate C228

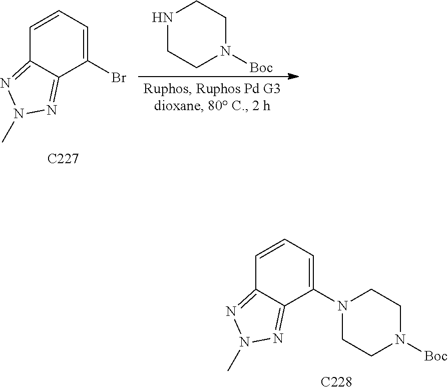 [SMILES: Cn1nc2cccc(Br)c2n1 ; CC(C)(C)OC(=O)N1CCNCC1 ; "C227" ; Cn1nc2cccc(N3CCN(C(=O)OC(C)(C)C)CC3)c2n1 ; "C228"]

To a solution of 4-bromo-2-methyl-1,2,3-benzotriazole (0.8 g, 3.773 mmol, 1.0 equiv) and tert-butyl piperazine-1-carboxylate (0.9 g, 4.905 mmol, 1.3 equiv) in dioxane (10 mL) were added Cs₂CO₃ (3.0 g, 9.433 mmol, 2.5 equiv) and Ruphos (0.4 g, 0.755 mmol, 0.2 equiv), RuPhos Palladacycle Gen.3 (0.2 g, 0.377 mmol, 0.1 equiv). After stirring for 2 hr at 80° C. under a nitrogen atmosphere, the resulting mixture was concentrated under reduced pressure. The residue was purified by silica gel column chromatography, eluted with PE/EA (1:1) to afford tert-butyl 4-(2-methyl-1,2,3-benzotriazol-4-yl) piperazine-1-carboxylate (C228, 1.2 g, 90%) as a solid. LCMS (ES, m/z): 318 [M+H]⁺

### Synthesis of Intermediate C229

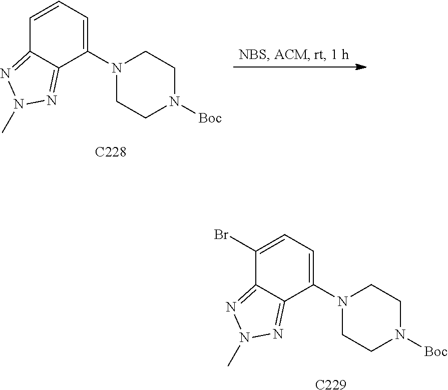 [SMILES: Cn1nc2cccc(N3CCN(C(=O)OC(C)(C)C)CC3)c2n1 ; "C228" ; Cn1nc2c(Br)ccc(N3CCN(C(=O)OC(C)(C)C)CC3)c2n1 ; "C229"]

To a stirred solution of tert-butyl 4-(2-methyl-1,2,3-benzotriazol-4-yl) piperazine-1-carboxylate (850.0 mg, 2.678 mmol, 1.0 equiv) in ACN (15 mL) were added NBS (524.3 mg, 2.946 mmol, 1.1 equiv) in portions at room temperature. The resulting mixture was stirred for 1 hr at room temperature. The resulting mixture was diluted with water (30 mL). The resulting mixture was extracted with ethyl acetate (2×40 mL). The combined organic layers were washed with water (2×30 mL), dried over anhydrous Na₂SO₄. After filtration, the filtrate was concentrated under reduced pressure. The residue was purified by silica gel column chromatography, eluted with PE/EA (1:1) to afford tert-butyl 4-(7-bromo-2-methyl-1,2,3-benzotriazol-4-yl) piperazine-1-carboxylate (C229, 820.0 mg, 73%) as a solid. LCMS (ES, m/z): 396 [M+H]⁺

### Synthesis of Intermediate C230

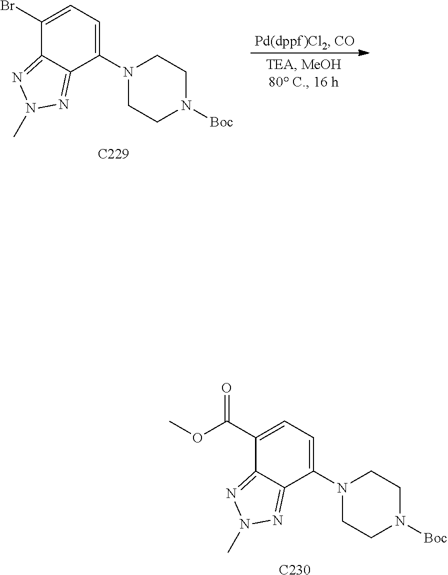

To a solution of tert-butyl 4-(7-bromo-2-methyl-1,2,3-benzotriazol-4-yl) piperazine-1-carboxylate (250.0 mg, 0.631 mmol, 1.0 equiv) in MeOH (20 mL) was added Pd(dppf)Cl₂ (46.1 mg, 0.063 mmol, 0.1 equiv), TEA (191.5 mg, 1.893 mmol, 3.0 equiv) in a pressure tank. The mixture was purged with nitrogen for 2 min and then was pressurized to 2 Mpa with carbon monoxide at 80° C. for 16 h. The reaction mixture was cooled to room temperature and filtered to remove insoluble solids. The resulting mixture was concentrated under reduced pressure. The residue was purified by silica gel column chromatography, eluted with CH₂Cl₂/MeOH (30:1) to afford methyl 7-[4-(tert-butoxycarbonyl) piperazin-1-yl]-2-methyl-1,2,3-benzotriazole-4-carboxylate as a solid. LCMS (ES, m/z): 376 [M+H]⁺

### Synthesis of Intermediate C231

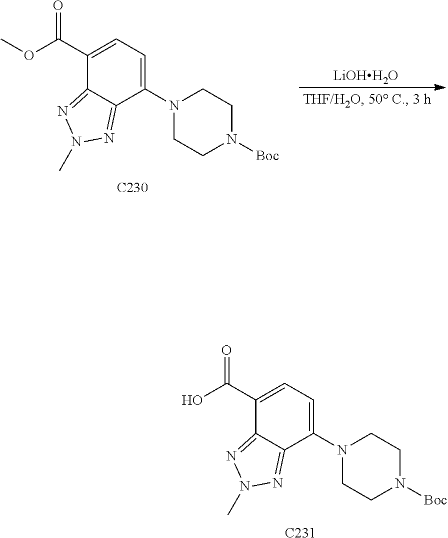

To a stirred mixture of methyl 7-[4-(tert-butoxycarbonyl) piperazin-1-yl]-2-methyl-1,2,3-benzotriazole-4-carboxylate (170.0 mg, 0.453 mmol, 1.0 equiv) in tetrahydrofuran (3 mL) and water (3 mL) was added LiOH·H₂O (108.4 mg, 4.530 mmol, 10.0 equiv) in portions at room temperature. The resulting mixture was stirred for 3 hr at 50° C. The resulting mixture was diluted with deionized water (20 mL). The mixture was acidified to pH 6 with HCl (1 N). The resulting mixture was extracted with ethyl acetate (2×30 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 7-[4-(tert-butoxycarbonyl) piperazin-1-yl]-2-methyl-1,2,3-benzotriazole-4-carboxylic acid (C231, 130 mg, 73%) as a solid. LCMS (ES, m/z): 362 [M+H]⁺

### Synthesis of Intermediate C232

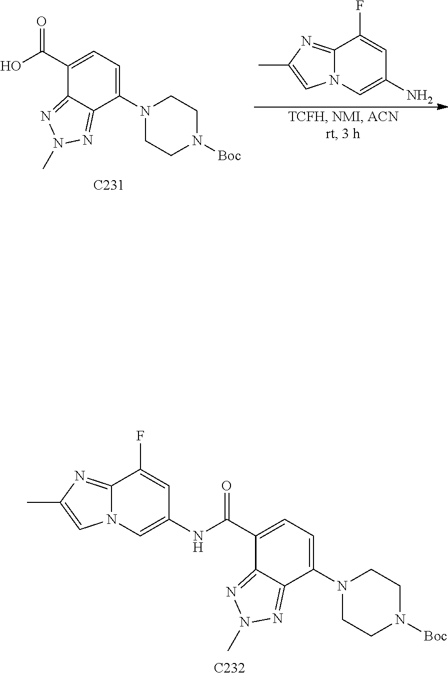

To a stirred solution of 7-[4-(tert-butoxycarbonyl)piperazin-1-yl]-2-methyl-1,2,3-benzotriazole-4-carboxylic acid (110.0 mg, 0.304 mmol, 1.0 equiv) and 8-fluoro-2-methylimidazo[1,2-a]pyridin-6-amine (75.4 mg, 0.456 mmol, 1.5 equiv) in ACN (3 mL) were added TCFH (111.0 mg, 0.395 mmol, 1.3 equiv) and NMI (64.8 mg, 0.790 mmol, 2.6 equiv) in portions at room temperature. The resulting mixture was stirred for 3 hr at room temperature. The resulting mixture was extracted with ethyl acetate (2×20 mL). The combined organic layers were washed with brine (2×20 mL), dried over anhydrous Na₂SO₄. After filtration, the filtrate was concentrated under reduced pressure. This resulted in tert-butyl 4-[7-({8-fluoro-2-methylimidazo[1,2-a]pyridin-6-yl}carbamoyl)-2-methyl-1,2,3-benzotriazol-4-yl]piperazine-1-carboxylate (C232, 100.0 mg, 58%) as a solid. LCMS (ES, m/z): 509 [M+H]⁺

### Synthesis of Compound 427

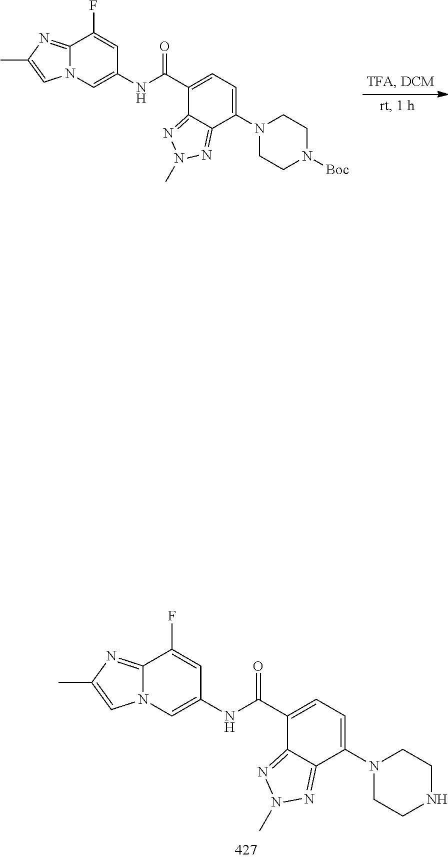 [SMILES: Cc1cn2cc(NC(=O)c3ccc(N4CCN(C(=O)OC(C)(C)C)CC4)c4nn(C)nc34)cc(F)c2n1 ; Cc1cn2cc(NC(=O)c3ccc(N4CCNCC4)c4nn(C)nc34)cc(F)c2n1]

To a stirred solution of tert-butyl 4-[7-({8-fluoro-2-methylimidazo[1,2-a]pyridin-6-yl}carbamoyl)-2-methyl-1,2,3-benzotriazol-4-yl]piperazine-1-carboxylate (100.0 mg, 0.197 mmol, 1.0 equiv) in DCM (2 mL) was added TFA (0.5 mL) dropwise at room temperature. The resulting mixture was stirred for 1 hr at room temperature. The resulting mixture was concentrated under reduced pressure. The crude product was purified by Prep-HPLC (Condition 13, Gradient 1) to afford N-{8-fluoro-2-methylimidazo[1,2-a]pyridin-6-yl}-2-methyl-7-(piperazin-1-yl)-1,2,3-benzotriazole-4-carboxamide (Compound 427, 19.7 mg, 23%) as a solid. LCMS (ES, m/z): 409 [M+H]^{+ 1}H NMR (400 MHz, DMSO-d₆) δ 10.18 (s, 1H), 9.19 (d, J=1.6 Hz, 1H), 8.00 (d, J=8.3 Hz, 1H), 7.91 (d, J=3.1 Hz, 1H), 7.39 (dd, J=12.5, 1.7 Hz, 1H), 6.72 (d, J=8.4 Hz, 1H), 4.61 (s, 3H), 3.72 (t, J=5.0 Hz, 4H), 2.91 (t, J=5.1 Hz, 4H), 2.35 (s, 3H).

### Example 169: Synthesis of Compound 428

### Synthesis of Intermediate C233

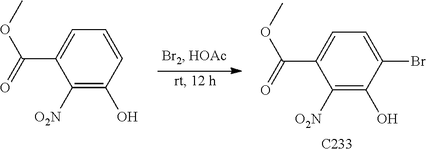

To a solution of methyl 3-hydroxy-2-nitrobenzoate (2 g, 10.145 mmol, 1 equiv) in HOAc (40 mL) was added with Br₂ (2.4 g, 15.018 mmol, 1.48 equiv) dropwise at 0° C. The resulting was stirred for 12 h at room temperature. The reaction was quenched with aq. Na₂S₂O₃ (50 mL) at room temperature. The resulting mixture was extracted with EA (3×50 mL). The combined organic layers were washed with brine (1×20 mL), dried over anhydrous Na₂SO₄. After filtration, the filtrate was concentrated under reduced pressure. The residue was purified by silica gel column chromatography, eluted with PE:EA (4:1) to afford methyl 4-bromo-3-hydroxy-2-nitrobenzoate (C233, 1 g, 35%) as a solid. LCMS (ES, m/z): 276 [M+H]⁺

### Synthesis of Intermediate C234

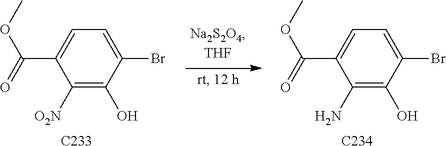

To a stirred solution of methyl 4-bromo-3-hydroxy-2-nitrobenzoate (1 g, 3.623 mmol, 1 equiv) in THF (15 mL) was added a solution of Na₂S₂O₄ (3.15 g, 18.115 mmol, 5.0 equiv) in H₂O (15 mL) dropwise at room temperature. The resulting mixture was stirred at room temperature for 12 h. The resulting mixture was extracted with EA (3×20 mL). The combined organic layers were washed with brine (1×10 mL), dried over anhydrous Na₂SO₄. After filtration, the filtrate was concentrated under reduced pressure. The residue was purified by silica gel column chromatography, eluted with PE:EA (4:1) to afford methyl 2-amino-4-bromo-3-hydroxybenzoate (C234, 500 mg, 56%) as a solid. LCMS (ES, m/z): 246 [M+H]⁺

### Synthesis of Intermediate C235

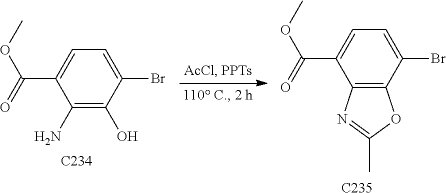

A solution of methyl 2-amino-4-bromo-3-hydroxybenzoate (400 mg, 1.626 mmol, 1 equiv) in toluene (25 mL) was treated with AcCl (153 mg, 1.951 mmol, 1.2 equiv), TEA (197.40 mg, 1.951 mmol, 1.2 equiv) and PPTS (122 mg, 0.488 mmol, 0.3 equiv) for 2 hr at 110° C. under nitrogen atmosphere. The resulting mixture was diluted with H₂O (10 mL). The resulting mixture was extracted with EA (2×10 mL). The combined organic layers were washed with NaCl (1×20 mL), dried over anhydrous Na₂SO₄. After filtration, the filtrate was concentrated under reduced pressure. The residue was purified by silica gel column chromatography, eluted with PE:EA (4:1) to afford methyl 7-bromo-2-methyl-1,3-benzoxazole-4-carboxylate (C235, 280 mg, 63%) as a solid. LCMS (ES, m/z): 270 [M+H]⁺

### Synthesis of Intermediate C236

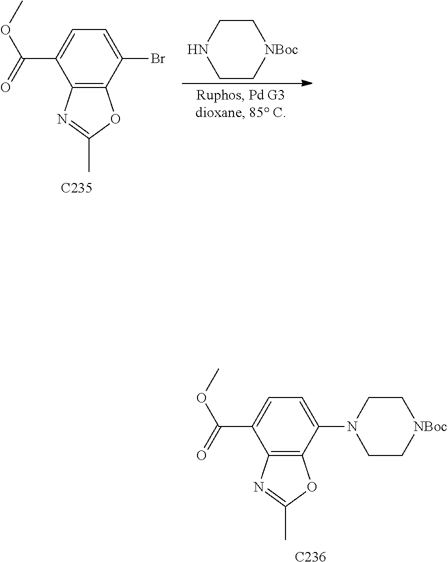 [SMILES: COC(=O)c1ccc(Br)c2oc(C)nc12 ; CC(C)(C)OC(=O)N1CCNCC1 ; "C235" ; COC(=O)c1ccc(N2CCN(C(=O)OC(C)(C)C)CC2)c2oc(C)nc12 ; "C236"]

To a solution of methyl 7-bromo-2-methyl-1,3-benzoxazole-4-carboxylate (280 mg, 1.037 mmol, 1 equiv) and tert-butyl piperazine-1-carboxylate (289 mg, 1.555 mmol, 1.5 equiv) in dioxane (5 mL) were added Cs₂CO₃ (1.01 g, 3.111 mmol, 3.0 equiv), RuPhos (96 mg, 0.207 mmol, 0.2 equiv) and RuPhos Palladacycle Gen.3 (48 mg, 0.104 mmol, 0.1 equiv). After stirring for 1 hr at 85° C. under a nitrogen atmosphere, the resulting mixture was concentrated under reduced pressure. The residue was purified by reverse flash chromatography (Condition 3, Gradient 1) to afford methyl 7-[4-(tert-butoxycarbonyl)piperazin-1-yl]-2-methyl-1,3-benzoxazole-4-carboxylate (C236, 200 mg, 51%) as a solid.

LCMS (ES, m/z): 376 [M+H]⁺

### Synthesis of Intermediate C237

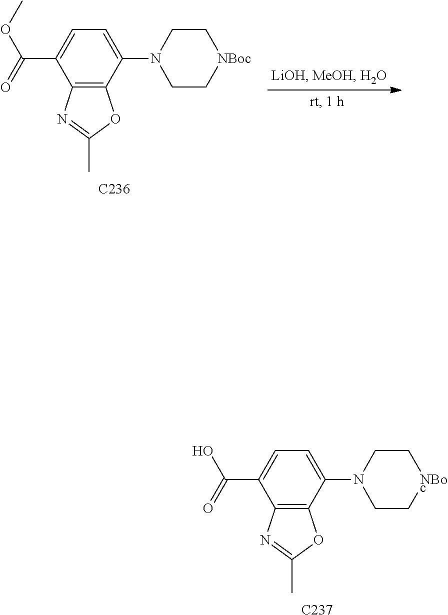 [SMILES: COC(=O)c1ccc(N2CCN(C(=O)OC(C)(C)C)CC2)c2oc(C)nc12 ; "C236" ; Cc1nc2c(C(=O)O)ccc(N3CCN(C(=O)OC(C)(C)C)CC3)c2o1 ; "C237"]

A solution of methyl 7-[4-(tert-butoxycarbonyl)piperazin-1-yl]-2-methyl-1,3-benzoxazole-4-carboxylate (250 mg, 0.666 mmol, 1 equiv) in MeOH (1 mL) and THF (1 mL) was treated with a solution of LiOH·H₂O (167 mg, 3.996 mmol, 6 equiv) in H₂O (1 mL) for 1 hr at room temperature. The mixture was acidified to pH 3 with 1 M HCl. The resulting mixture was extracted with EA (2×5 mL). The combined organic layers were washed with brine (1×5 mL), dried over anhydrous Na₂SO₄. After filtration, the filtrate was concentrated under reduced pressure. The residue was purified by reverse flash chromatography (Condition 3, Gradient 1) to afford 7-[4-(tert-butoxycarbonyl)piperazin-1-yl]-2-methyl-1,3-benzoxazole-4-carboxylic acid (C237, 200 mg, 83%) as a solid. LCMS (ES, m/z): 362 [M+H]⁺

### Synthesis of Intermediate C238

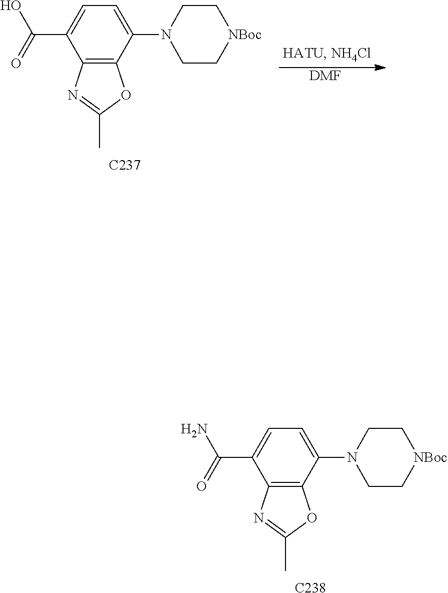

To a stirred solution of 7-[4-(tert-butoxycarbonyl)piperazin-1-yl]-2-methyl-1,3-benzoxazole-4-carboxylic acid (240 mg, 0.664 mmol, 1 equiv) and HATU (757 mg, 1.992 mmol, 3.0 equiv) in DMF (7 mL) were added DIEA (257 mg, 1.992 mmol, 3.0 equiv) and NH₄Cl (355.22 mg, 6.640 mmol, 10 equiv) in portions at room temperature. The resulting mixture was stirred for 12 hr at room temperature. The residue was purified by reverse flash chromatography (Condition 3, Gradient 1) to afford tert-butyl 4-(4-carbamoyl-2-methyl-1,3-benzoxazol-7-yl) piperazine-1-carboxylate (C238, 150 mg, 62%) as a solid. LCMS (ES, m/z): 361 [M+H]⁺

### Synthesis of Intermediate C239

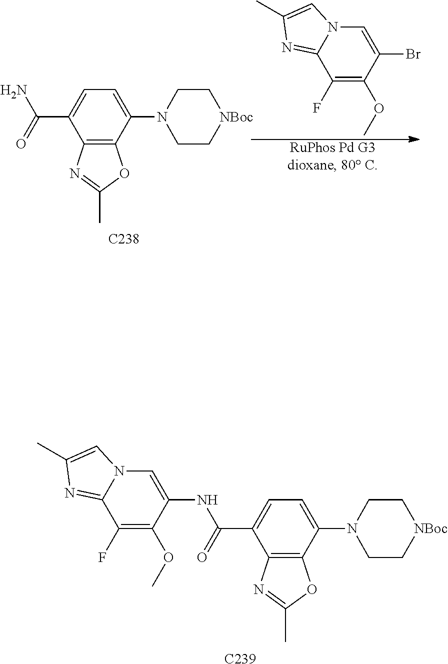

To a solution of tert-butyl 4-(4-carbamoyl-2-methyl-1,3-benzoxazol-7-yl)piperazine-1-carboxylate (50 mg, 0.139 mmol, 1 equiv) and 6-bromo-8-fluoro-7-methoxy-2-methylimidazo[1,2-a]pyridine (43 mg, 0.167 mmol, 1.2 equiv) in dioxane (1 mL) were added Cs₂CO₃ (135 mg, 0.417 mmol, 3.0 equiv), XantPhos (16 mg, 0.028 mmol, 0.2 equiv) and Pd₂(dba)₃CHCl₃ (14 mg, 0.014 mmol, 0.1 equiv). After stirring for 1 h at 80° C. under a nitrogen atmosphere, the resulting mixture was concentrated under reduced pressure. The residue was purified by silica gel column chromatography, eluted with PE:EA (1:1) to afford tert-butyl 4-[4-({8-fluoro-7-methoxy-2-methylimidazo[1,2-a]pyridin-6-yl}carbamoyl)-2-methyl-1,3-benzoxazol-7-yl]piperazine-1-carboxylate (C239, 70 mg, 93%) as a solid. LCMS (ES, m/z): 539 [M+H]⁺

### Synthesis of Compound 428

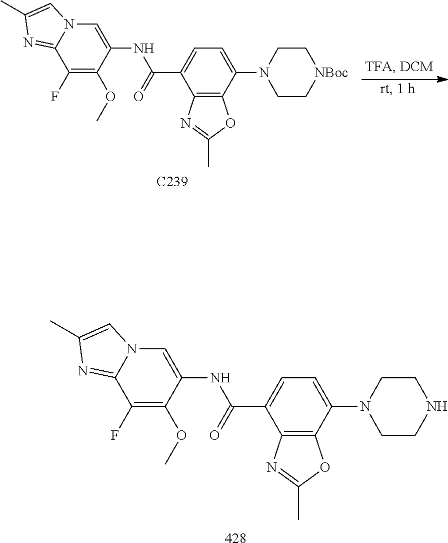 [SMILES: COc1c(NC(=O)c2ccc(N3CCN(C(=O)OC(C)(C)C)CC3)c3oc(C)nc23)cn2cc(C)nc2c1F ; "rt, 1 h" ; "C239" ; COc1c(NC(=O)c2ccc(N3CCNCC3)c3oc(C)nc23)cn2cc(C)nc2c1F ; "428"]

A solution of tert-butyl 4-[4-({8-fluoro-7-methoxy-2-methylimidazo[1,2-a]pyridine-6-yl}carbamoyl)-2-methyl-1,3-benzoxazol-7-yl]piperazine-1-carboxylate (60 mg, 0.111 mmol, 1 equiv) and TFA (0.3 mL) in DCM (2 mL) was stirred for 1 hr at room temperature. The mixture was basified to pH 8 with NH₃ in MeOH. The resulting mixture was concentrated under reduced pressure. The residue was purified by reverse flash chromatography (Condition 3, Gradient 5) to afford N-{8-fluoro-7-methoxy-2-methylimidazo[1,2-a]pyridin-6-yl}-2-methyl-7-(piperazin-1-yl)-1,3-benzoxazole-4-carboxamide (Compound 428, 14 mg, 28%) as a solid. LCMS (ES, m/z): 439 [M+H]^{+ 1}H NMR (300 MHz, DMSO-d₆) δ 11.32 (s, 1H), 9.43 (s, 1H), 7.92 (d, J=8.6 Hz, 1H), 7.80 (d, J=3.1 Hz, 1H), 6.95 (d, J=8.8 Hz, 1H), 4.20 (d, J=2.0 Hz, 3H), 3.48-3.39 (m, 4H), 2.90 (t, J=5.0 Hz, 4H), 2.80 (s, 3H), 2.32 (s, 3H).

### Example 170: Synthesis of Compound 429

### Synthesis of Intermediate C240

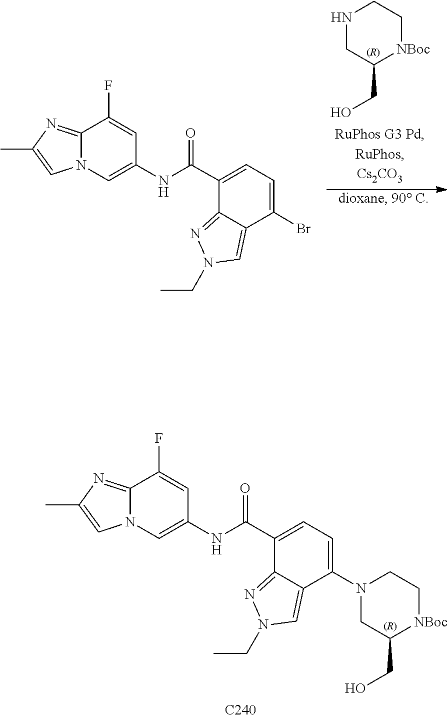

To a solution of 4-bromo-2-ethyl-N-{8-fluoro-2-methylimidazo[1,2-a]pyridin-6-yl}indazole-7-carboxamide (1 g, 2.402 mmol, 1 equiv) and tert-butyl (R)-2-(hydroxymethyl)piperazine-1-carboxylate (0.57 g, 2.642 mmol, 1.1 equiv) in dioxane (20 mL) were added Cs₂CO₃ (2.35 g, 7.206 mmol, 3.0 equiv), RuPhos Palladacycle Gen.3 (0.4 g, 0.480 mmol, 0.2 equiv) and RuPhos (0.22 g, 0.480 mmol, 0.2 equiv). After stirring for 3 hr at 90° C. under a nitrogen atmosphere. The resulting mixture was diluted with H₂O (20 mL). The resulting mixture was extracted with EA (3×20 mL). The combined organic layers were washed with NaCl (1×10 mL), dried over anhydrous Na₂SO₄. After filtration, the filtrate was concentrated under reduced pressure. The residue was purified by reverse flash chromatography (Condition 5, Gradient 2) to afford tert-butyl (R)-4-(2-ethyl-7-((8-fluoro-2-methylimidazo[1,2-a]pyridin-6-yl)carbamoyl)-2H-indazol-4-yl)-2-(hydroxymethyl)piperazine-1-carboxylate (C240, 500 mg, 37%) as a solid. LCMS (ES, m/z): 552 [M+H]

### Synthesis of Intermediate C241

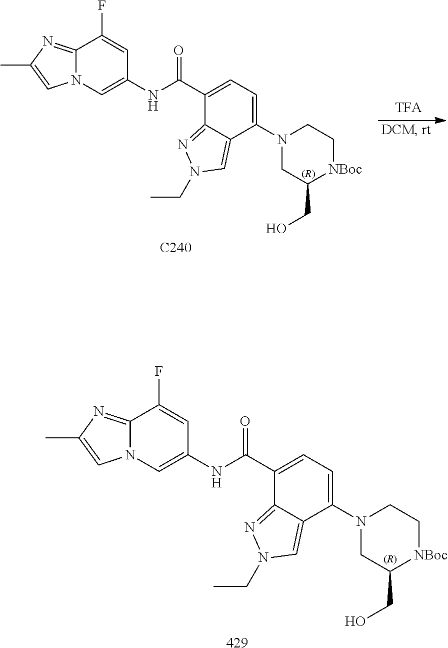

A solution of tert-butyl (R)-4-(2-ethyl-7-((8-fluoro-2-methylimidazo[1,2-a]pyridin-6-yl)carbamoyl)-2H-indazol-4-yl)-2-(hydroxymethyl)piperazine-1-carboxylate (50 mg, 0.091 mmol, 1 equiv) and TFA (0.2 mL) in DCM (2 mL) was stirred for 1 h at room temperature. The mixture was neutralized to pH 8 with ammonia in methanol. The resulting mixture was concentrated under reduced pressure. The residue was purified by reverse flash chromatography (Condition 3, Gradient 6) to afford (R)-2-ethyl-N-(8-fluoro-2-methylimidazo[1,2-a]pyridin-6-yl)-4-(3-(hydroxymethyl)piperazin-1-yl)-2H-indazole-7-carboxamide (Compound 429, 10 mg, 24%) as a solid. LCMS (ES, m/z): 452 [M+H]^{+ 1}H NMR (300 MHz, DMSO-d₆) δ 11.11 (s, 1H), 9.21 (d, J=1.7 Hz, 1H), 8.80 (s, 1H), 7.98 (d, J=8.1 Hz, 1H), 7.91 (dd, J=3.2, 1.0 Hz, 1H), 7.31 (dd, J=12.3, 1.7 Hz, 1H), 6.49 (d, J=8.2 Hz, 1H), 4.73 (s, 1H), 4.60 (q, J=7.3 Hz, 2H), 3.79 (d, J=10.5 Hz, 2H), 3.44 (s, 2H), 3.09-2.98 (m, 1H), 2.91 (q, J=10.3, 9.3 Hz, 3H), 2.77-2.63 (m, 1H), 2.39-2.32 (m, 3H), 1.62 (t, J=7.3 Hz, 3H).

### Example 171: Synthesis of Compound 430

### Synthesis of Intermediate C241

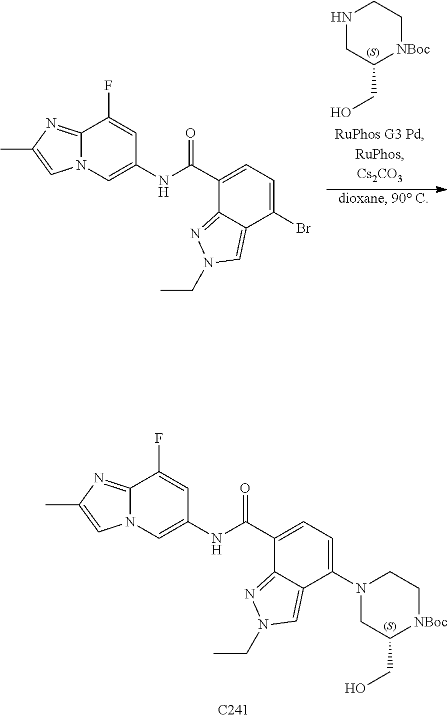

To a solution of 4-bromo-2-ethyl-N-{8-fluoro-2-methylimidazo[1,2-a]pyridin-6-yl}indazole-7-carboxamide (1 g, 2.402 mmol, 1 equiv) and tert-butyl (S)-2-(hydroxymethyl)piperazine-1-carboxylate (0.57 g, 2.642 mmol, 1.1 equiv) in dioxane (20 mL) were added Cs₂CO₃ (2.35 g, 7.206 mmol, 3.0 equiv), RuPhos Palladacycle Gen.3 (0.4 g, 0.480 mmol, 0.2 equiv) and RuPhos (0.22 g, 0.480 mmol, 0.2 equiv). After stirring for 3 hr at 90° C. under a nitrogen atmosphere. The resulting mixture was diluted with H₂O (20 mL). The resulting mixture was extracted with EA (3×20 mL). The combined organic layers were washed with NaCl (1×10 mL), dried over anhydrous Na₂SO₄. After filtration, the filtrate was concentrated under reduced pressure. The residue was purified by reverse flash chromatography (Condition 5, Gradient 2) to afford tert-butyl (S)-4-(2-ethyl-7-((8-fluoro-2-methylimidazo[1,2-a]pyridin-6-yl)carbamoyl)-2H-indazol-4-yl)-2-(hydroxymethyl)piperazine-1-carboxylate (500 mg, 37.73%) as a solid. LCMS (ES, m/z): 552 [M+H]

### Synthesis of Compound 430

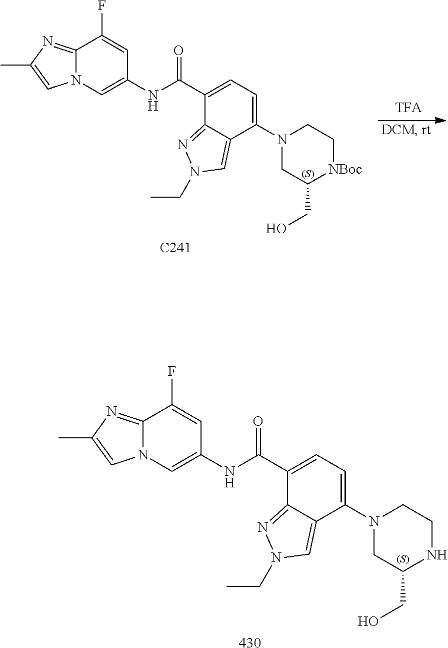

A solution of tert-butyl (S)-4-(2-ethyl-7-((8-fluoro-2-methylimidazo[1,2-a]pyridin-6-yl)carbamoyl)-2H-indazol-4-yl)-2-(hydroxymethyl)piperazine-1-carboxylate (50 mg, 0.091 mmol, 1 equiv) and TFA (0.2 mL) in DCM (2 mL) was stirred for 1 h at room temperature. The mixture was neutralized to pH 8 with ammonia in methanol. The resulting mixture was concentrated under reduced pressure. The residue was purified by reverse flash chromatography (Condition 5, Gradient 3) to afford (S)-2-ethyl-N-(8-fluoro-2-methylimidazo[1,2-a]pyridin-6-yl)-4-(3-(hydroxymethyl)piperazin-1-yl)-2H-indazole-7-carboxamide (Compound 430, 11 mg, 25%) as a solid. LCMS (ES, m/z): 452 [M+H]^{+ 1}H NMR (300 MHz, DMSO-d₆) δ 11.11 (s, 1H), 9.21 (d, J=1.7 Hz, 1H), 8.80 (s, 1H), 7.98 (d, J=8.1 Hz, 1H), 7.91 (dd, J=3.2, 1.0 Hz, 1H), 7.31 (dd, J=12.3, 1.7 Hz, 1H), 6.49 (d, J=8.2 Hz, 1H), 4.73 (s, 1H), 4.60 (q, J=7.3 Hz, 2H), 3.79 (d, J=10.5 Hz, 2H), 3.44 (s, 2H), 3.09-2.98 (m, 1H), 2.91 (q, J=10.3, 9.3 Hz, 3H), 2.77-2.63 (m, 1H), 2.39-2.32 (m, 3H), 1.62 (t, J=7.3 Hz, 3H).

### Example 172: Synthesis of Compound 469

### Synthesis of Intermediate C242

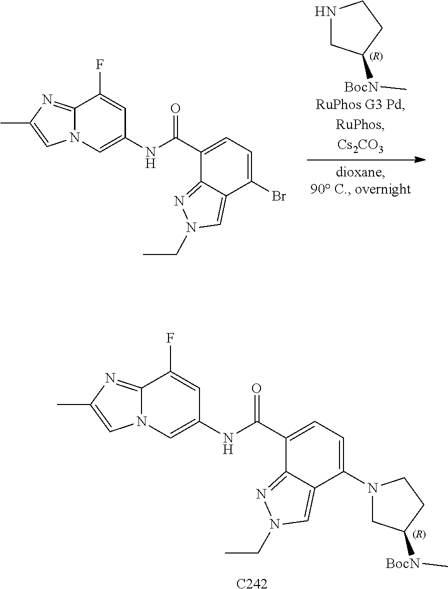

To a stirred solution of 4-bromo-2-ethyl-N-{8-fluoro-2-methylimidazo[1,2-a]pyridin-6-yl}indazole-7-carboxamide (180 mg, 0.43 mmol, 1 equiv) and tert-butyl (R)-methyl(pyrrolidin-3-yl)carbamate (87 mg, 0.43 mmol, 1 equiv) in dioxane (10 mL) were added Cs₂CO₃ (423 mg, 1.29 mmol, 3 equiv), RuPhos (41 mg, 0.086 mmol, 0.2 equiv) and RuPhos Palladacycle Gen.3 (36 mg, 0.043 mmol, 0.1 equiv) in portions at room temperature under nitrogen atmosphere. The resulting mixture was stirred for overnight at 90° C. under nitrogen atmosphere. The resulting mixture was concentrated under reduced pressure. The resulting mixture was added H₂O (20 mL) and extracted with ethyl acetate (3×10 mL). The combined organic layers were washed with brine (1×30 mL), dried over anhydrous Na₂SO₄. After filtration, the filtrate was concentrated under reduced pressure. The residue was purified by reverse flash chromatography (Condition 5, Gradient 2) to afford tert-butyl (R)-(1-(2-ethyl-7-((8-fluoro-2-methylimidazo[1,2-a]pyridin-6-yl)carbamoyl)-2H-indazol-4-yl)pyrrolidin-3-yl)(methyl)carbamate (C242, 160 mg, 69%) as a solid. LCMS (ES, m/z): 536 [M+H]⁺

### Synthesis of Compound 469

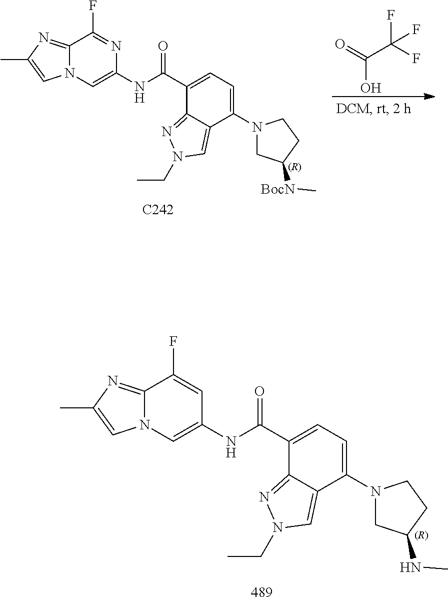

A solution of tert-butyl tert-butyl (R)-(1-(2-ethyl-7-((8-fluoro-2-methylimidazo[1,2-a]pyridin-6-yl)carbamoyl)-2H-indazol-4-yl)pyrrolidin-3-yl)(methyl)carbamate (135 mg, 0.25 mmol, 1 equiv) in trifluoroacetic acid (2 mL) and DCM (2 mL) was stirred for 2 hr at room temperature. The resulting mixture was concentrated under reduced pressure. The residue was basified to pH 8 with 7 M NH₃(g) in MeOH. The resulting mixture was concentrated under reduced pressure. The residue was purified by prep-HPLC (Condition 12, Gradient 3) to afford (R)-2-ethyl-N-(8-fluoro-2-methylimidazo[1,2-a]pyridin-6-yl)-4-(3-(methylamino)pyrrolidin-1-yl)-2H-indazole-7-carboxamide (Compound 469, 12 mg, 10%) as a solid. LCMS (ES, m/z): 436 [M+H]^{+ 1}H NMR (300 MHz, DMSO-d6) δ 11.05 (s, 1H), 9.19 (d, J=1.6 Hz, 1H), 8.84 (s, 1H), 7.97-7.85 (m, 2H), 7.26 (dd, J=12.4, 1.6 Hz, 1H), 6.01 (d, J=8.4 Hz, 1H), 4.57 (q, J=7.3 Hz, 2H), 3.76 (dq, J=13.7, 7.1, 6.3 Hz, 1H), 3.65 (d, J=7.4 Hz, 3H), 3.42 (dd, J=10.2, 4.0 Hz, 2H), 2.35 (s, 6H), 2.14 (dd, J=11.2, 4.5 Hz, 1H), 1.92 (dd, J=11.8, 6.1 Hz, 1H), 1.61 (t, J=7.2 Hz, 3H).

### Example 173: Synthesis of Compound 437

### Synthesis of Intermediate C243

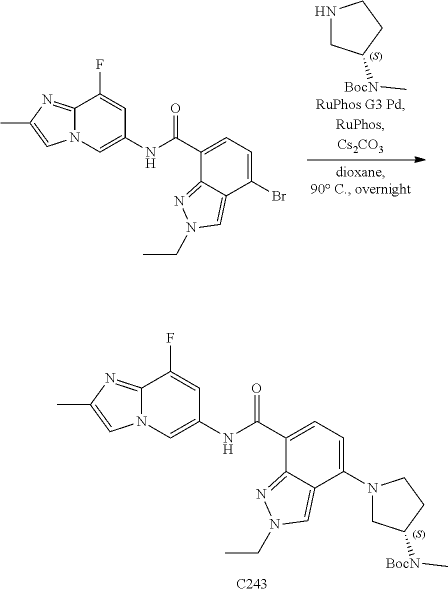

To a stirred solution of 4-bromo-2-ethyl-N-{8-fluoro-2-methylimidazo[1,2-a]pyridin-6-yl}indazole-7-carboxamide (180 mg, 0.43 mmol, 1 equiv) and tert-butyl (S)-methyl(pyrrolidin-3-yl)carbamate (87 mg, 0.43 mmol, 1 equiv) in dioxane (10 mL) were added Cs₂CO₃ (423 mg, 1.29 mmol, 3 equiv), RuPhos (41 mg, 0.086 mmol, 0.2 equiv) and RuPhos Palladacycle Gen.3 (36 mg, 0.043 mmol, 0.1 equiv) in portions at room temperature under nitrogen atmosphere. The resulting mixture was stirred for overnight at 90° C. under nitrogen atmosphere. The resulting mixture was concentrated under reduced pressure. The resulting mixture was added H₂O (20 mL) and extracted with ethyl acetate (3×10 mL). The combined organic layers were washed with brine (1×30 mL), dried over anhydrous Na₂SO₄. After filtration, the filtrate was concentrated under reduced pressure. The residue was purified by reverse flash chromatography (Condition 5, Gradient 2) to afford tert-butyl (S)-(1-(2-ethyl-7-((8-fluoro-2-methylimidazo[1,2-a]pyridin-6-yl)carbamoyl)-2H-indazol-4-yl)pyrrolidin-3-yl)(methyl)carbamate (C243, 160 mg, 69%) as a solid. LCMS (ES, m/z): 536 [M+H]⁺

### Synthesis of Compound 437

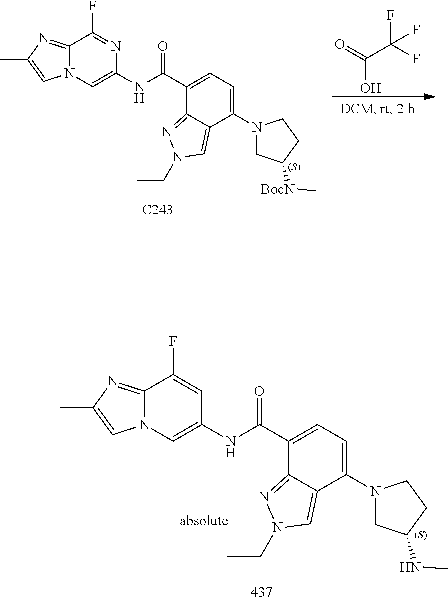

A solution of tert-butyl tert-butyl (S)-(1-(2-ethyl-7-((8-fluoro-2-methylimidazo[1,2-a]pyridin-6-yl)carbamoyl)-2H-indazol-4-yl)pyrrolidin-3-yl)(methyl)carbamate (135 mg, 0.25 mmol, 1 equiv) in trifluoroacetic acid (2 mL) and DCM (2 mL) was stirred for 2 h at room temperature. The resulting mixture was concentrated under reduced pressure. The residue was basified to pH 8 with 7 M NH₃(g) in MeOH. The resulting mixture was concentrated under reduced pressure. The residue was purified by prep-HPLC (Condition 12, Gradient 3) to afford (S)-2-ethyl-N-(8-fluoro-2-methylimidazo[1,2-a]pyridin-6-yl)-4-(3-(methylamino)pyrrolidin-1-yl)-2H-indazole-7-carboxamide (Compound 437, 13 mg, 11%) as a solid. LCMS (ES, m/z): 436 [M+H]^{+ 1}H NMR (300 MHz, DMSO-d6) δ 11.05 (s, 1H), 9.19 (d, J=1.6 Hz, 1H), 8.84 (s, 1H), 7.97-7.85 (m, 2H), 7.26 (dd, J=12.4, 1.6 Hz, 1H), 6.01 (d, J=8.4 Hz, 1H), 4.57 (q, J=7.3 Hz, 2H), 3.76 (dq, J=13.7, 7.1, 6.3 Hz, 1H), 3.65 (d, J=7.4 Hz, 3H), 3.42 (dd, J=10.2, 4.0 Hz, 2H), 2.35 (s, 6H), 2.14 (dd, J=11.2, 4.5 Hz, 1H), 1.92 (dd, J=11.8, 6.1 Hz, 1H), 1.61 (t, J=7.2 Hz, 3H).

### Example 174: Synthesis of Compounds 438 and 439

### Synthesis of Compound 439

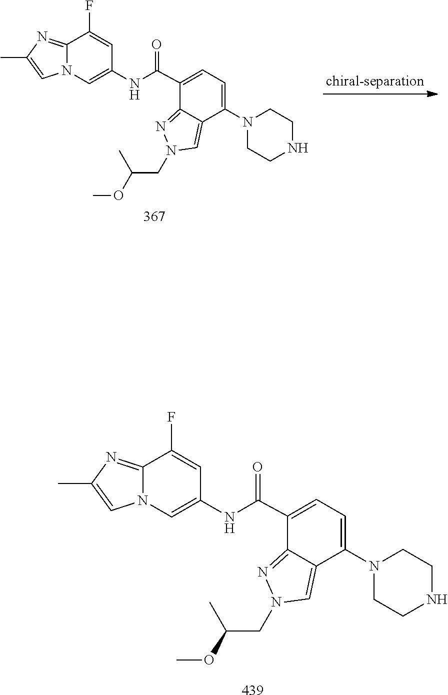

Compound 367 was separated by prep-chiral-HPLC (Condition 6, Gradient 1) to afford (S)—N-(8-fluoro-2-methylimidazo[1,2-a]pyridin-6-yl)-2-(2-methoxypropyl)-4-(piperazin-1-yl)-2H-indazole-7-carboxamide (Compound 438, 1.7 mg) as a solid. LCMS (ES, m/z): 466 [M+H]^{+ 1}H NMR (400 MHz, Methanol-d₄) δ 9.10 (d, J=1.7 Hz, 1H), 8.52 (s, 1H), 8.11 (d, J=8.0 Hz, 1H), 7.73 (d, J=3.0 Hz, 1H), 7.26 (dd, J=11.8, 1.7 Hz, 1H), 6.55 (d, J=8.2 Hz, 1H), 4.72-4.33 (m, 2H), 4.02 (qd, J=6.5, 3.7 Hz, 1H), 3.46 (dd, J=6.3, 3.7 Hz, 4H), 3.33 (p, J=1.6 Hz, 3H), 3.08 (dd, J=6.1, 3.6 Hz, 4H), 2.44 (s, 3H), 1.29 (d, J=6.3 Hz, 3H).

### Synthesis of Compound 438

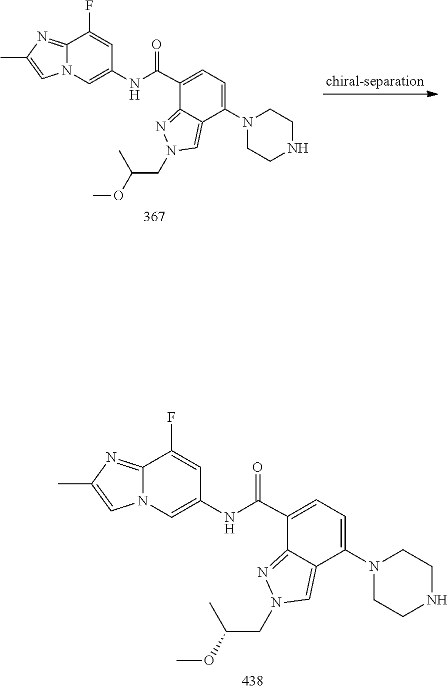

Compound 367 (17 mg) was separated by prep-chiral-HPLC (Condition 6, Gradient 1) to afford (R)—N-(8-fluoro-2-methylimidazo[1,2-a]pyridin-6-yl)-2-(2-methoxypropyl)-4-(piperazin-1-yl)-2H-indazole-7-carboxamide (Compound 438, 1.2 mg) as a solid. LCMS (ES, m/z): 466 [M+H]^{+ 1}H NMR (400 MHz, Methanol-d₄) δ 9.10 (d, J=1.7 Hz, 1H), 8.52 (s, 1H), 8.11 (d, J=8.0 Hz, 1H), 7.73 (d, J=3.0 Hz, 1H), 7.26 (dd, J=11.8, 1.7 Hz, 1H), 6.55 (d, J=8.2 Hz, 1H), 4.72-4.33 (m, 2H), 4.02 (qd, J=6.5, 3.7 Hz, 1H), 3.46 (dd, J=6.3, 3.7 Hz, 4H), 3.33 (p, J=1.6 Hz, 3H), 3.08 (dd, J=6.1, 3.6 Hz, 4H), 2.44 (s, 3H), 1.29 (d, J=6.3 Hz, 3H).

### Example 175: Synthesis of Compound 443

### Synthesis of Intermediate C244

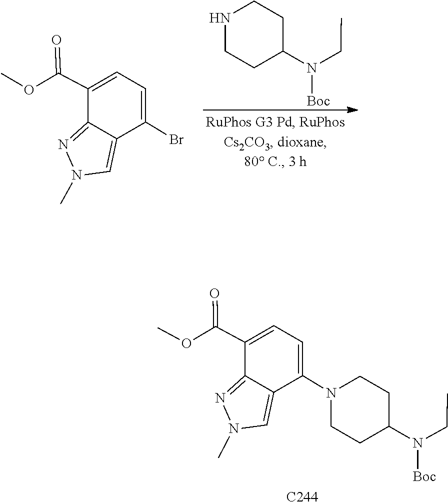 [SMILES: COC(=O)c1ccc(Br)c2cn(C)nc12 ; CCN(C(=O)OC(C)(C)C)C1CCNCC1 ; CCN(C(=O)OC(C)(C)C)C1CCN(c2ccc(C(=O)OC)c3nn(C)cc23)CC1 ; "C244"]

To a solution of methyl 4-bromo-2-methylindazole-7-carboxylate (300.0 mg, 1.115 mmol, 1.0 equiv) and tert-butyl N-ethyl-N-(piperidin-4-yl) carbamate (305.4 mg, 1.338 mmol, 1.2 equiv) in dioxane (10 mL) were added Cs₂CO₃ (910.9 mg, 2.788 mmol, 2.5 equiv) and Ruphos (104.0 mg, 0.223 mmol, 0.2 equiv), RuPhos Palladacycle Gen.3 (93.2 mg, 0.112 mmol, 0.1 equiv). After stirring for 3 h at 80° C. under nitrogen atmosphere, the resulting mixture was concentrated under reduced pressure. The residue was purified by silica gel column chromatography, eluted with CH₂Cl₂/MeOH (20:1) to afford methyl 4-{4-[(tert-butoxycarbonyl) (ethyl)amino] piperidin-1-yl}-2-methylindazole-7-carboxylate (C244, 320 mg, 63%) as a solid. LCMS (ES, m/z): 417 [M+H]⁺

### Synthesis of Intermediate C245

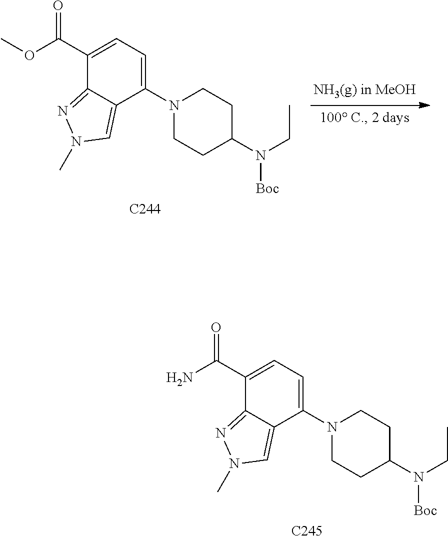 [SMILES: CCN(C(=O)OC(C)(C)C)C1CCN(c2ccc(C(=O)OC)c3nn(C)cc23)CC1 ; "C244" ; CCN(C(=O)OC(C)(C)C)C1CCN(c2ccc(C(N)=O)c3nn(C)cc23)CC1 ; "C245"]

To a solution of methyl 4-{4-[(tert-butoxycarbonyl) (ethyl)amino] piperidin-1-yl}-2-methylindazole-7-carboxylate (300.0 mg, 0.720 mmol, 1.0 equiv) was added NH₃(g) in MeOH (50 mL) in a pressure tank. The resulting mixture was stirred for 2 days at 100° C. The resulting mixture was concentrated under reduced pressure. The residue was purified by silica gel column chromatography, eluted with CH₂Cl₂/MeOH (30:1) to afford tert-butyl N-[1-(7-carbamoyl-2-methylindazol-4-yl) piperidin-4-yl]-N-ethylcarbamate (C245, 280 mg, 87%) as a solid. LCMS (ES, m/z): 402 [M+H]⁺

### Synthesis of Intermediate C246

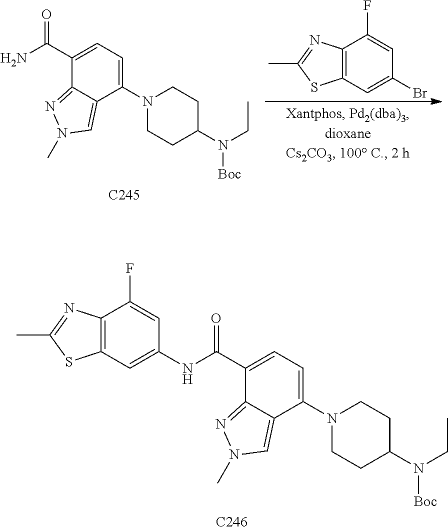

To a solution of tert-butyl N-[1-(7-carbamoyl-2-methylindazol-4-yl) piperidin-4-yl]-N-ethylcarbamate (130.0 mg, 0.324 mmol, 1.0 equiv) and 6-bromo-4-fluoro-2-methyl-1,3-benzothiazole (95.6 mg, 0.389 mmol, 1.2 equiv) in dioxane (4 mL) were added Cs₂CO₃ (264.5 mg, 0.810 mmol, 2.5 equiv) and Xantphos (37.4 mg, 0.065 mmol, 0.2 equiv), Pd₂(dba)₃ (29.6 mg, 0.032 mmol, 0.1 equiv). After stirring for 2 hr at 100° C. under nitrogen atmosphere, the resulting mixture was concentrated under reduced pressure. The residue was purified by silica gel column chromatography, eluted with CH₂Cl₂/MeOH (30:1) to afford tert-butyl N-ethyl-N-(1-{7-[(4-fluoro-2-methyl-1,3-benzothiazol-6-yl) carbamoyl]-2-methylindazol-4-yl} piperidin-4-yl)carbamate (C246, 100.0 mg, 54%) as a solid. LCMS (ES, m/z): 567 [M+H]⁺

### Synthesis of Compound 443

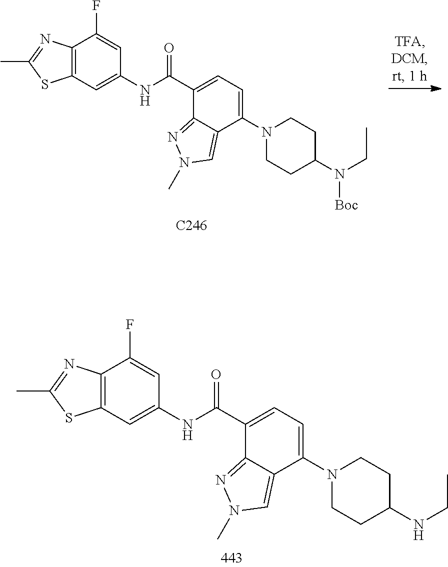

To a stirred solution of tert-butyl N-ethyl-N-(1-{7-[(4-fluoro-2-methyl-1,3-benzothiazol-6-yl)carbamoyl]-2-methylindazol-4-yl}piperidin-4-yl)carbamate (110.0 mg, 0.194 mmol, 1.0 equiv) in DCM (2 mL) was added TFA (0.5 mL) dropwise at room temperature. The resulting mixture was stirred for 1 hr at room temperature under nitrogen atmosphere. The reaction was monitored by LCMS. The crude product was purified by Prep-HPLC (Condition 13, Gradient 1) to afford 4-[4-(ethylamino) piperidin-1-yl]-N-(4-fluoro-2-methyl-1,3-benzothiazol-6-yl)-2-methylindazole-7-carboxamide (Compound 443, 27 mg, 29%) as a solid. LCMS (ES, m/z): 467 [M+H]^{+ 1}H NMR (400 MHz, DMSO-d₆) δ 11.43 (s, 1H), 8.78 (s, 1H), 8.30 (d, J=1.9 Hz, 1H), 7.99 (d, J=8.2 Hz, 1H), 7.89 (dd, J=12.9, 1.9 Hz, 1H), 6.50 (d, J=8.3 Hz, 1H), 4.31 (s, 3H), 3.91 (d, J=12.9 Hz, 2H), 3.07 (t, J=11.4 Hz, 2H), 2.81 (s, 3H), 2.73-2.72 (m, 1H), 2.63 (t, J=7.1 Hz, 2H), 1.97 (d, J=12.4 Hz, 2H), 1.46 (q, J=11.0 Hz, 2H), 1.05 (t, J=7.1 Hz, 3H).

### Example 176: Synthesis of Compound 444

### Synthesis of Intermediate C247

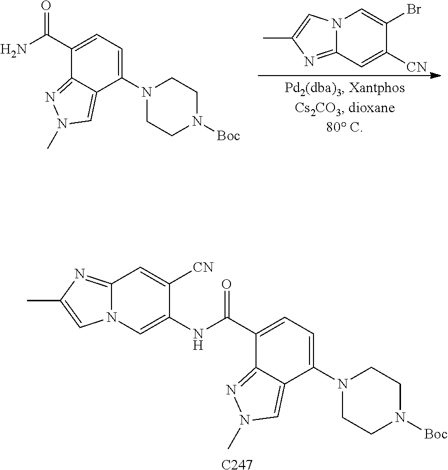 [SMILES: Cn1cc2c(N3CCN(C(=O)OC(C)(C)C)CC3)ccc(C(N)=O)c2n1 ; Cc1cn2cc(Br)c(C#N)cc2n1 ; Cc1cn2cc(NC(=O)c3ccc(N4CCN(C(=O)OC(C)(C)C)CC4)c4cn(C)nc34)c(C#N)cc2n1 ; "C247"]

To a solution of tert-butyl 4-(7-carbamoyl-2-methylindazol-4-yl) piperazine-1-carboxylate (300 mg, 0.835 mmol, 1 equiv) and 6-bromo-2-methylimidazo[1,2-a]pyridine-7-carbonitrile (197.04 mg, 0.835 mmol, 1 equiv) in dioxane (5 mL) were added Cs₂CO₃ (815.84 mg, 2.505 mmol, 3 equiv), Pd₂(dba)₃ (76.43 mg, 0.084 mmol, 0.1 equiv) and XantPhos (48.30 mg, 0.084 mmol, 0.1 equiv). After stirring for 2 hr at 100° C. under a nitrogen atmosphere. The resulting mixture was concentrated under reduced pressure. The residue was purified by silica gel column chromatography, eluted with PE:EA (1:1-1:10) to afford tert-butyl 4-[7-({7-cyano-2-methylimidazo[1,2-a]pyridin-6-yl}carbamoyl)-2-methylindazol-4-yl]piperazine-1-carboxylate (C247, 400 mg, 93%) as a solid. ¹H NMR (400 MHz, DMSO-d₆) δ 11.47 (s, 1H), 9.66 (s, 1H), 8.85 (s, 1H), 8.29 (s, 1H), 8.06-7.96 (m, 2H), 6.52 (d, J=8.2 Hz, 1H), 4.26 (s, 3H), 3.57 (d, J=5.7 Hz, 5H), 3.48 (d, J=5.7 Hz, 3H), 2.40 (s, 3H), 1.45 (s, 9H).

### Synthesis of Compound 444

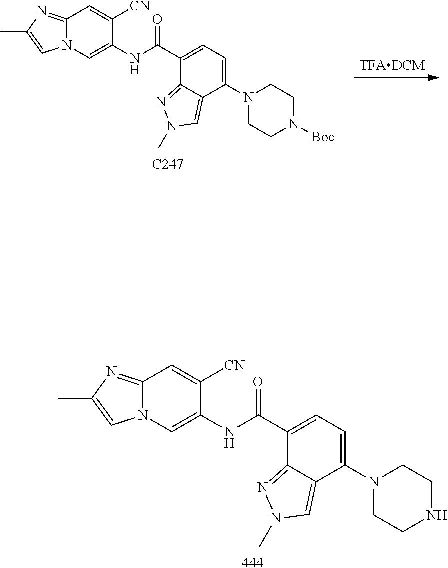

A solution of tert-butyl 4-[7-({7-cyano-2-methylimidazo[1,2-a]pyridin-6-yl}carbamoyl)-2-methylindazol-4-yl]piperazine-1-carboxylate (80 mg, 0.155 mmol, 1 equiv) in DCM (4 mL) was added TFA (1 mL) dropwise at room temperature. The resulting mixture was stirred for 2 h at room temperature. The resulting mixture was concentrated under reduced pressure. The residue was purified by reverse flash chromatography (Condition 5, Gradient 4) to afford N-{7-cyano-2-methylimidazo[1,2-a]pyridin-6-yl}-2-methyl-4-(piperazin-1-yl) indazole-7-carboxamide (Compound 444, 4 mg, 6%) as a solid. LCMS (ES, m/z): 515 [M+H]^{+ 1}H NMR (400 MHz, Methanol-d₄) δ 11.48 (s, 1H), 9.67 (s, 1H), 8.82 (s, 1H), 8.29 (s, 1H), 8.08-7.97 (m, 2H), 6.51 (d, J=8.2 Hz, 1H), 4.25 (s, 3H), 3.39 (t, J=5.0 Hz, 4H), 2.91 (t, J=5.0 Hz, 4H), 2.40 (s, 3H).

### Example 177: Synthesis of Compound 445

### Synthesis of Intermediate C248

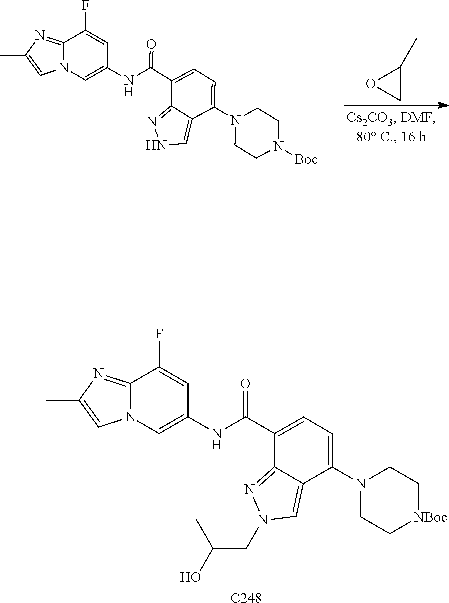

To a stirred mixture of tert-butyl 4-[7-({8-fluoro-2-methylimidazo[1,2-a]pyridin-6-yl}carbamoyl)-2H-indazol-4-yl]piperazine-1-carboxylate (300.0 mg, 0.608 mmol, 1.0 equiv) and Cs₂CO₃ (396.1 mg, 1.216 mmol, 2.0 equiv) in DMF (6 mL) was added propylene oxide (52.9 mg, 0.912 mmol, 1.5 equiv) dropwise at room temperature. The resulting mixture was stirred for 16 h at 80° C. The resulting mixture was diluted with water (20 mL). The resulting mixture was extracted with ethyl acetate (2×20 mL). The combined organic layers were washed with water (2×20 mL), dried over anhydrous Na₂SO₄. After filtration, the filtrate was concentrated under reduced pressure. The residue was purified by silica gel column chromatography, eluted with PE/EA (1:9) to afford tert-butyl 4-[7-({8-fluoro-2-methylimidazo[1,2-a]pyridin-6-yl}carbamoyl)-2-(2-hydroxypropyl) indazol-4-yl]piperazine-1-carboxylate (C248, 200 mg, 59%) as a solid. LCMS (ES, m/z): 552 [M+H]⁺

### Synthesis of Intermediate C249

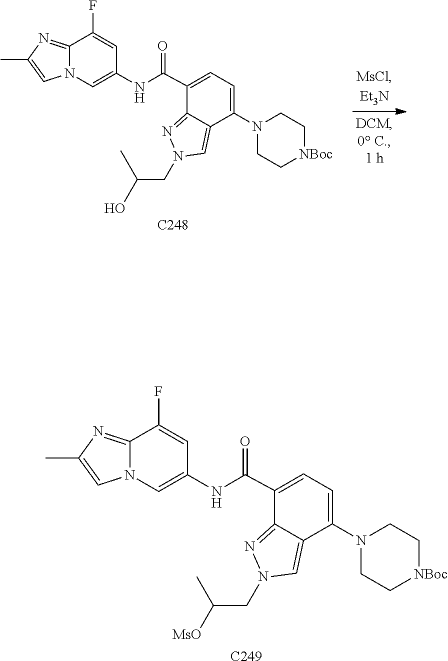 [SMILES: Cc1cn2cc(NC(=O)c3ccc(N4CCN(C(=O)OC(C)(C)C)CC4)c4cn(CC(C)O)nc34)cc(F)c2n1 ; "C248" ; Cc1cn2cc(NC(=O)c3ccc(N4CCN(C(=O)OC(C)(C)C)CC4)c4cn(CC(C)OS(C)(=O)=O)nc34)cc(F)c2n1 ; "C249"]

To a stirred mixture of tert-butyl 4-[7-({8-fluoro-2-methylimidazo[1,2-a]pyridin-6-yl}carbamoyl)-2-(2-hydroxypropyl) indazol-4-yl]piperazine-1-carboxylate (210.0 mg, 0.381 mmol, 1.0 equiv) and Et₃N (77.0 mg, 0.762 mmol, 2.0 equiv) in DCM (4 mL) was added MsCl (52.3 mg, 0.457 mmol, 1.2 equiv) dropwise at 0° C. The resulting mixture was stirred for 1 hr at 0° C. The resulting mixture was washed with 1×4 mL of water. The organic layer was dried over anhydrous Na₂SO₄. After filtration, the filtrate was concentrated under reduced pressure. The resulting mixture was concentrated under vacuum to afford tert-butyl 4-[7-({8-fluoro-2-methylimidazo[1,2-a]pyridin-6-yl}carbamoyl)-2-[2-(methanesulfonyloxy)propyl]indazol-4-yl]piperazine-1-carboxylate (C249, 200 mg, 83%) as a solid. LCMS (ES, m/z): 630 [M+H]⁺

### Synthesis of Intermediate C250

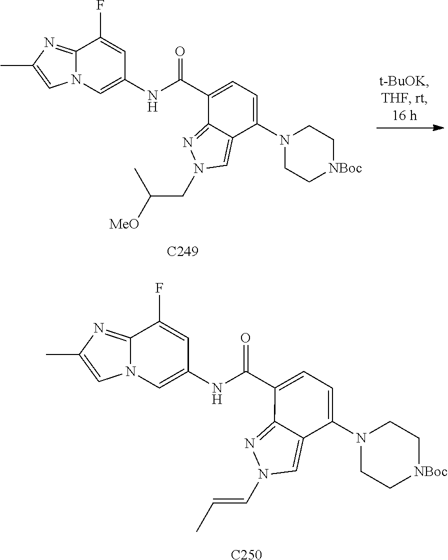 [SMILES: COC(C)Cn1cc2c(N3CCN(C(=O)OC(C)(C)C)CC3)ccc(C(=O)Nc3cc(F)c4nc(C)cn4c3)c2n1 ; "C249" ; C/C=C/n1cc2c(N3CCN(C(=O)OC(C)(C)C)CC3)ccc(C(=O)Nc3cc(F)c4nc(C)cn4c3)c2n1 ; "C250"]

To a stirred mixture of tert-butyl 4-[7-({8-fluoro-2-methylimidazo[1,2-a]pyridin-6-yl}carbamoyl)-2-[2-(methanesulfonyloxy)propyl]indazol-4-yl]piperazine-1-carboxylate (210.0 mg, 0.333 mmol, 1.0 equiv) in THF (2 mL) was added t-BuOK (74.8 mg, 0.666 mmol, 2.0 equiv) at room temperature. The resulting mixture was stirred for 16 hr at room temperature. The resulting mixture was diluted with water (4 mL). The resulting mixture was extracted with ethyl acetate (2×4 mL). The combined organic layers were washed with water (1×4 mL), dried over anhydrous Na₂SO₄. After filtration, the filtrate was concentrated under reduced pressure. The residue was purified by silica gel column chromatography, eluted with PE/EA (1:9) to afford tert-butyl 4-[7-({8-fluoro-2-methylimidazo[1,2-a]pyridin-6-yl}carbamoyl)-2-[(1E)-prop-1-en-1-yl]indazol-4-yl]piperazine-1-carboxylate (C250, 130 mg, 73%) as a solid. LCMS (ES, m/z): 534 [M+H]⁺

### Synthesis of Compound 445

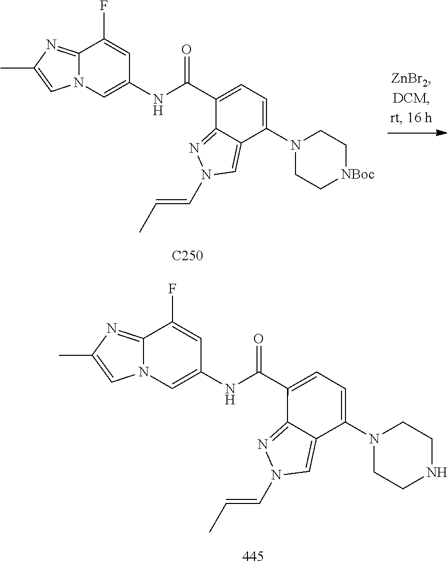 [SMILES: C/C=C/n1cc2c(N3CCN(C(=O)OC(C)(C)C)CC3)ccc(C(=O)Nc3cc(F)c4nc(C)cn4c3)c2n1 ; "C250" ; C/C=C/n1cc2c(N3CCNCC3)ccc(C(=O)Nc3cc(F)c4nc(C)cn4c3)c2n1 ; "445"]

To a stirred mixture of tert-butyl 4-[7-({8-fluoro-2-methylimidazo[1,2-a]pyridin-6-yl}carbamoyl)-2-[(1E)-prop-1-en-1-yl]indazol-4-yl]piperazine-1-carboxylate (80.0 mg, 0.150 mmol, 1.0 equiv) in DCM (2 mL) was added ZnBr₂ (337.6 mg, 1.500 mmol, 10.0 equiv) at room temperature. The resulting mixture was stirred for 16 hr at room temperature. The resulting mixture was concentrated under vacuum. The crude product was purified by Prep-HPLC (Condition 10, Gradient 9) to afford N-{8-fluoro-2-methylimidazo[1,2-a]pyridin-6-yl}-4-(piperazin-1-yl)-2-[(1E)-prop-1-en-1-yl]indazole-7-carboxamide (Compound 445, 18 mg, 27%) as a solid.

LCMS (ES, m/z): 434 [M+H]^{+ 1}H NMR (400 MHz, DMSO-d₆) δ 10.91 (s, 1H), 9.25-9.15 (m, 1H), 8.98 (s, 1H), 8.01 (d, J=8.1 Hz, 1H), 7.90 (d, J=3.1 Hz, 1H), 7.47 (dd, J=14.1, 1.9 Hz, 1H), 7.38-7.29 (m, 1H), 6.78 (dq, J=14.0, 6.9 Hz, 1H), 6.50 (d, J=8.2 Hz, 1H), 3.37 (t, J=5.1 Hz, 4H), 2.92 (t, J=5.0 Hz, 4H), 2.36 (s, 3H), 1.96 (dd, J=7.0, 1.8 Hz, 3H).

### Example 178: Synthesis of Compound 446 and 447

### Synthesis of Intermediate C251

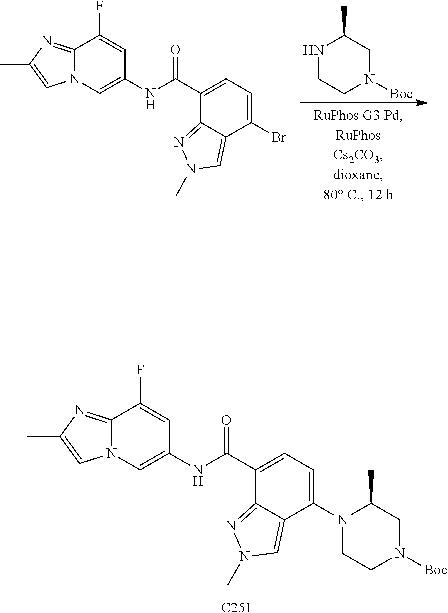 [SMILES: Cc1cn2cc(NC(=O)c3ccc(Br)c4cn(C)nc34)cc(F)c2n1 ; C[C@H]1CN(C(=O)OC(C)(C)C)CCN1 ; Cc1cn2cc(NC(=O)c3ccc(N4CCN(C(=O)OC(C)(C)C)C[C@@H]4C)c4cn(C)nc34)cc(F)c2n1 ; "C251"]

To a stirred mixture of 4-bromo-N-{8-fluoro-2-methylimidazo[1,2-a]pyridin-6-yl}-2-methylindazole-7-carboxamide (100 mg, 0.249 mmol, 1 equiv) and tert-butyl (3S)-3-methylpiperazine-1-carboxylate (59.75 mg, 0.299 mmol, 1.2 equiv) in dioxane (2 mL) was added Cs₂CO₃ (243.01 mg, 0.747 mmol, 3 equiv), RuPhos (23.20 mg, 0.050 mmol, 0.2 equiv) and RuPhos Palladacycle Gen.3 (20.79 mg, 0.025 mmol, 0.1 equiv) at room temperature under nitrogen atmosphere. The resulting mixture was stirred for 12 hr at 80° C. under nitrogen atmosphere. The resulting mixture was concentrated under reduced pressure. The residue was purified by silica gel column chromatography, eluted with CH₂Cl₂/MeOH (10:1) to afford tert-butyl (3S)-4-[7-({8-fluoro-2-methylimidazo[1,2-a]pyridin-6-yl}carbamoyl)-2-methylindazol-4-yl]-3-methylpiperazine-1-carboxylate (C251, 70 mg, 53%) as solid. LCMS (ES, m/z): 522 [M+H]⁺

### Synthesis of Compound 446

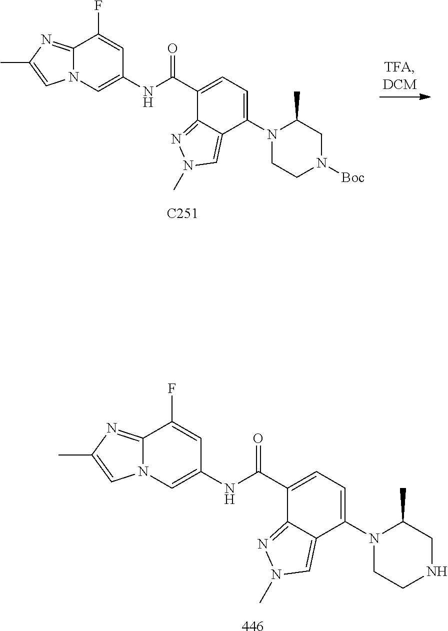

To a stirred solution of tert-butyl (3S)-4-[7-({8-fluoro-2-methylimidazo[1,2-a]pyridin-6-yl}carbamoyl)-2-methylindazol-4-yl]-3-methylpiperazine-1-carboxylate (70 mg, 0.134 mmol, 1 equiv) in DCM (1 mL) was added TFA (1 mL) at room temperature. The resulting mixture was stirred for 2 h at room temperature. The resulting mixture was concentrated under reduced pressure. The crude product was purified by Prep-HPLC (Condition 10, Gradient 5) to afford N-{8-fluoro-2-methylimidazo[1,2-a]pyridin-6-yl}-2-methyl-4-[(2S)-2-methylpiperazin-1-yl]indazole-7-carboxamide (Compound 44614.9 mg, 26%) as solid. LCMS (ES, m/z): 422 [M+H]^{+ 1}H NMR (400 MHz, Methanol-d₄) δ 9.11 (d, J=1.6 Hz, 1H), 8.55 (s, 1H), 8.11 (d, J=8.1 Hz, 1H), 7.74 (d, J=3.0 Hz, 1H), 7.33-7.25 (m, 1H), 6.57 (d, J=8.2 Hz, 1H), 4.34 (s, 3H), 4.28 (dd, J=6.8, 3.4 Hz, 1H), 3.49-3.37 (m, 2H), 3.25 (dd, J=12.8, 3.9 Hz, 1H), 3.16 (d, J=12.9 Hz, 1H), 3.03-2.90 (m, 2H), 2.45 (d, J=0.9 Hz, 3H), 1.20 (d, J=6.7 Hz, 3H).

### Synthesis of Intermediate C252

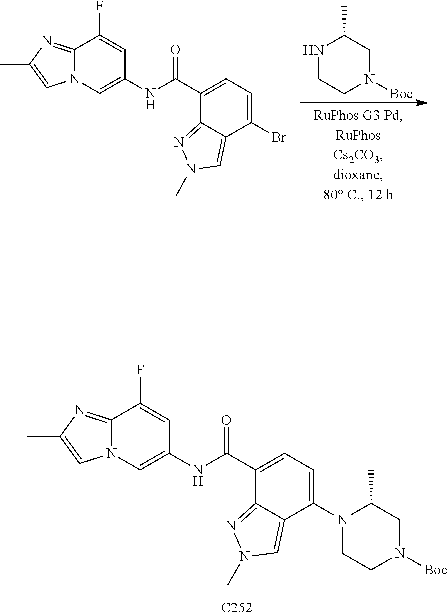 [SMILES: Cc1cn2cc(NC(=O)c3ccc(Br)c4cn(C)nc34)cc(F)c2n1 ; C[C@@H]1CN(C(=O)OC(C)(C)C)CCN1 ; Cc1cn2cc(NC(=O)c3ccc(N4CCN(C(=O)OC(C)(C)C)C[C@H]4C)c4cn(C)nc34)cc(F)c2n1 ; "C252"]

To a stirred mixture of 4-bromo-N-{8-fluoro-2-methylimidazo[1,2-a]pyridin-6-yl}-2-methylindazole-7-carboxamide (100 mg, 0.249 mmol, 1 equiv) and tert-butyl (3R)-3-methylpiperazine-1-carboxylate (59.75 mg, 0.299 mmol, 1.2 equiv) in dioxane (2 mL) was added Cs₂CO₃ (243.01 mg, 0.747 mmol, 3 equiv), RuPhos (23.20 mg, 0.050 mmol, 0.2 equiv) and RuPhos Palladacycle Gen.3 (20.79 mg, 0.025 mmol, 0.1 equiv) at room temperature under nitrogen atmosphere. The resulting mixture was stirred for 12 hr at 80° C. under nitrogen atmosphere. The resulting mixture was concentrated under reduced pressure. The residue was purified by silica gel column chromatography, eluted with CH₂Cl₂/MeOH (10:1) to afford tert-butyl (3R)-4-[7-({8-fluoro-2-methylimidazo[1,2-a]pyridin-6-yl}carbamoyl)-2-methylindazol-4-yl]-3-methylpiperazine-1-carboxylate (C252, 80 mg, 61%) as solid. LCMS (ES, m/z): 522 [M+H]⁺

### Synthesis of Compound 447

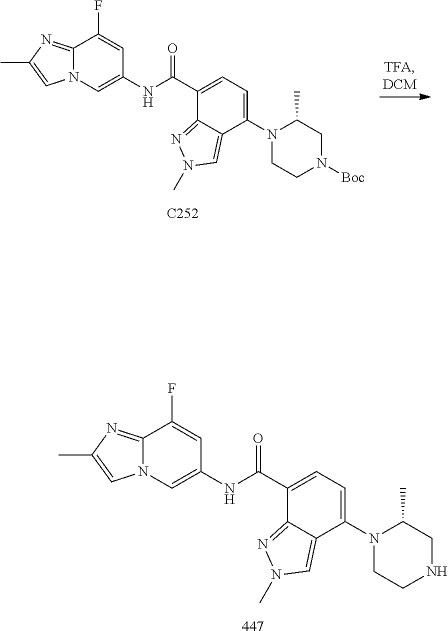

To a stirred solution of tert-butyl (3R)-4-[7-({8-fluoro-2-methylimidazo[1,2-a]pyridin-6-yl}carbamoyl)-2-methylindazol-4-yl]-3-methylpiperazine-1-carboxylate (80 mg, 0.153 mmol, 1 equiv) in DCM (1 mL) was added TFA (1 mL) at room temperature. The resulting mixture was stirred for 2 h at room temperature. The resulting mixture was concentrated under reduced pressure. The crude product was purified by Prep-HPLC (Condition 10, Gradient 5) to afford N-{8-fluoro-2-methylimidazo[1,2-a]pyridin-6-yl}-2-methyl-4-[(2R)-2-methylpiperazin-1-yl]indazole-7-carboxamide (Compound 447, 21.6 mg, 33%) as solid. LCMS (ES, m/z): 422 [M+H]^{+ 1}H NMR (400 MHz, Methanol-d₄) δ 9.12-9.05 (m, 1H), 8.52 (d, J=4.8 Hz, 1H), 8.09 (dd, J=8.2, 3.1 Hz, 1H), 7.72 (s, 1H), 7.30-7.21 (m, 1H), 6.55 (dd, J=8.1, 3.8 Hz, 1H), 4.33 (d, J=2.1 Hz, 3H), 4.26 (s, 1H), 3.43 (q, J=14.7, 13.4 Hz, 2H), 3.28-3.19 (m, 1H), 3.15 (d, J=12.6 Hz, 1H), 3.03-2.89 (m, 2H), 2.46-2.41 (m, 3H), 1.19 (d, J=6.7 Hz, 3H).

### Example 179: Synthesis of Compound 455

### Synthesis of Intermediate C253

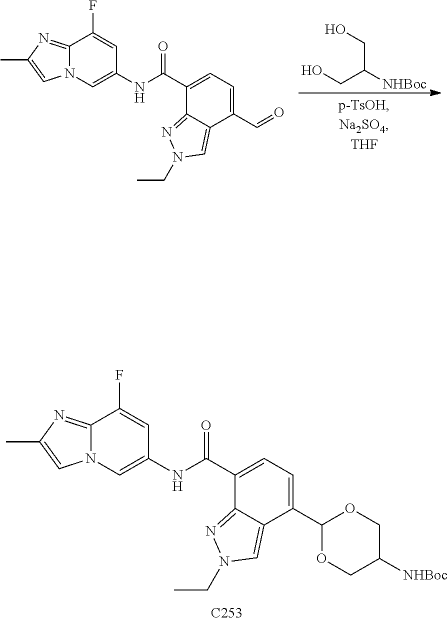 [SMILES: CCn1cc2c(C=O)ccc(C(=O)Nc3cc(F)c4nc(C)cn4c3)c2n1 ; CC(C)(C)OC(=O)NC(CO)CO ; CCn1cc2c(C3OCC(NC(=O)OC(C)(C)C)CO3)ccc(C(=O)Nc3cc(F)c4nc(C)cn4c3)c2n1 ; "C253"]

To a stirred solution of 2-ethyl-N-{8-fluoro-2-methylimidazo[1,2-a]pyridin-6-yl}-4-formylindazole-7-carboxamide (70 mg, 0.192 mmol, 1 equiv) and tert-butyl N-(1,3-dihydroxypropan-2-yl)carbamate (73 mg, 0.384 mmol, 2.0 equiv) in DCM (3.5 mL) and THF (0.3 mL) were added p-TsOH (50 mg, 0.288 mmol, 1.5 equiv) and Na₂SO₄ (41 mg, 0.288 mmol, 1.5 equiv) in portions at room temperature. The resulting mixture was stirred for 12 hr at room temperature. The resulting mixture was diluted with H₂O (5 mL). The resulting mixture was extracted with EA (3×5 mL). The combined organic layers were washed with brine (1×5 mL), dried over anhydrous Na₂SO₄. After filtration, the filtrate was concentrated under reduced pressure. The residue was purified by reverse flash chromatography (Condition 3, Gradient 1) to afford tert-butyl N-{2-[2-ethyl-7-({8-fluoro-2-methylimidazo[1,2-a]pyridin-6-yl}carbamoyl) indazol-4-yl]-1,3-dioxan-5-yl}carbamate (C253, 30 mg, 29%) as a solid. LCMS (ES, m/z): 539 [M+H]⁺

### Synthesis of Compound 455

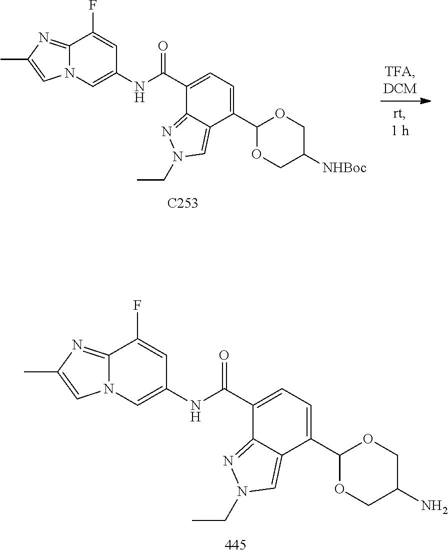

A solution of tert-butyl N-{2-[2-ethyl-7-({8-fluoro-2-methylimidazo[1,2-a]pyridin-6-yl}carbamoyl) indazol-4-yl]-1,3-dioxan-5-yl}carbamate (30 mg, 0.056 mmol, 1 equiv) and TFA (0.2 mL) in DCM (2 mL) was stirred for 1 h at room temperature. The mixture was basified to pH 8 with NH₃ in MeOH. The resulting mixture was concentrated under reduced pressure. The residue was purified by reverse flash chromatography (Condition 3, Gradient 2) to afford 4-(5-amino-1,3-dioxan-2-yl)-2-ethyl-N-{8-fluoro-2-methylimidazo[1,2-a]pyridin-6-yl}indazole-7-carboxamide (Compound 445, 10 mg, 40%) as a solid. LCMS (ES, m/z): 439 [M+H]^{+ 1}H NMR (300 MHz, DMSO-d₆) δ 11.22 (d, J=3.6 Hz, 1H), 9.25 (d, J=1.7 Hz, 1H), 8.81 (s, 1H), 8.08 (dd, J=7.3, 4.0 Hz, 1H), 7.95 (d, J=3.2 Hz, 1H), 7.41-7.28 (m, 2H), 5.87 (s, 1H), 4.76-4.62 (m, 2H), 4.18 (t, J=9.7 Hz, 2H), 3.93 (d, J=11.1 Hz, 2H), 3.51 (t, J=10.6 Hz, 1H), 2.77 (s, 1H), 2.36 (s, 3H), 1.69-1.58 (m, 3H).

### Example 180: Synthesis of Compound 460

### Synthesis of Intermediate C254

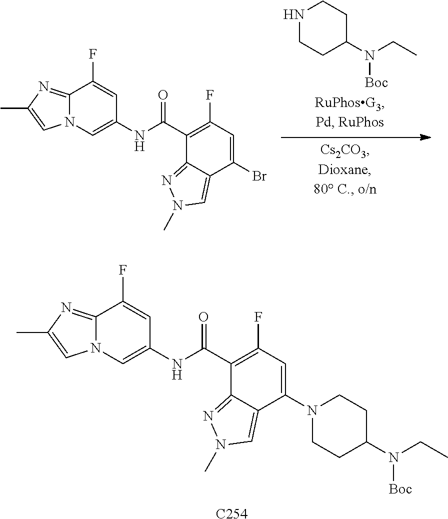 [SMILES: Cc1cn2cc(NC(=O)c3c(F)cc(Br)c4cn(C)nc34)cc(F)c2n1 ; CCN(C(=O)OC(C)(C)C)C1CCNCC1 ; CCN(C(=O)OC(C)(C)C)C1CCN(c2cc(F)c(C(=O)Nc3cc(F)c4nc(C)cn4c3)c3nn(C)cc23)CC1 ; "C254"]

To a stirred mixture of 4-bromo-6-fluoro-N-{8-fluoro-2-methylimidazo[1,2-a]pyridin-6-yl}-2-methylindazole-7-carboxamide (100 mg, 0.238 mmol, 1 equiv) and tert-butyl N-ethyl-N-(piperidin-4-yl)carbamate (54.34 mg, 0.238 mmol, 1 equiv), Cs₂CO₃ (227.19 mg, 0.696 mmol, 3 equiv) in 1,4-dioxane (2 mL) was added Ruphos (11.10 mg, 0.024 mmol, 0.1 equiv) and RuPhos Palladacycle Gen.3 (19.90 mg, 0.024 mmol, 0.1 equiv) at room temperature under nitrogen atmosphere. The resulting mixture was stirred overnight at 80° C. under nitrogen atmosphere. The resulting mixture was concentrated under vacuum. The residue was purified by silica gel column chromatography, eluted with CH₂Cl₂/MeOH (20:1) to afford tert-butyl N-ethyl-N-{1-[6-fluoro-7-({8-fluoro-2-methylimidazo[1,2-a]pyridin-6-yl}carbamoyl)-2-methylindazol-4-yl]piperidin-4-yl}carbamate (C254, 80 mg, 59%) as a solid. LCMS (ES, m/z): 568 [M+H]⁺

### Synthesis of Compound 460

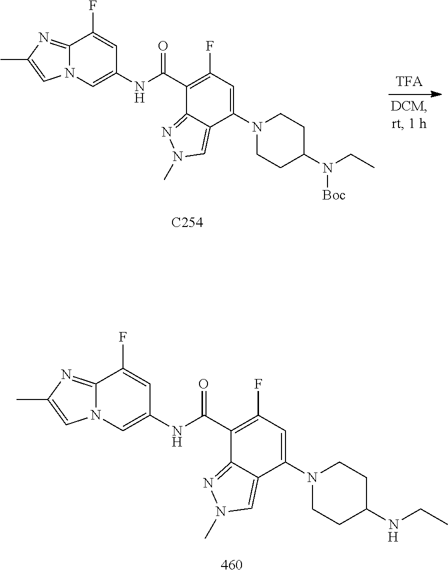

To a stirred solution of tert-butyl N-ethyl-N-{1-[6-fluoro-7-({8-fluoro-2-methylimidazo[1,2-a]pyridin-6-yl}carbamoyl)-2-methylindazol-4-yl]piperidin-4-yl}carbamate (70 mg, 0.123 mmol, 1 equiv) in DCM (1 mL) was added TFA (1 mL) at room temperature. The resulting mixture was stirred for 1 hr at room temperature. The resulting mixture was concentrated under vacuum. The residue was purified by Prep-HPLC (Condition 15, Gradient 2) to afford 4-[4-(ethylamino)piperidin-1-yl]-6-fluoro-N-{8-fluoro-2-methylimidazo[1,2-a]pyridin-6-yl}-2-methylindazole-7-carboxamide; bis(trifluoroacetic acid) (Compound 460, 22.9 mg, 26%) as solid. LCMS (ES, m/z): 468 [M+H]^{+ 1}H NMR (400 MHz, Methanol-d₄) δ 9.41 (s, 1H), 8.59 (s, 1H), 8.02 (s, 1H), 7.82 (t, J=9.5 Hz, 1H), 6.37 (d, J=15.4 Hz, 1H), 4.30 (s, 3H), 4.17 (d, J=13.2 Hz, 2H), 3.50-3.39 (m, 1H), 3.24-3.11 (m, 4H), 2.56 (s, 3H), 2.28 (d, J=12.2 Hz, 2H), 1.84 (tt, J=12.6, 6.4 Hz, 2H), 1.42-1.28 (m, 3H).

### Example 181: Synthesis of Compound 440

### Synthesis of Intermediate C255

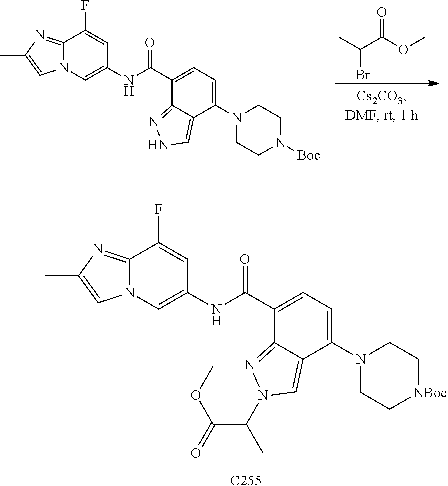

To a stirred mixture of tert-butyl 4-[7-({8-fluoro-2-methylimidazo[1,2-a]pyridin-6-yl}carbamoyl)-2H-indazol-4-yl]piperazine-1-carboxylate (800.0 mg, 1.621 mmol, 1.0 equiv) and methyl 2-bromopropanoate (406.0 mg, 2.431 mmol, 1.5 equiv) in DMF (20 mL) was added Cs₂CO₃ (1056.2 mg, 3.242 mmol, 2.0 equiv) at room temperature. The resulting mixture was stirred for 1 hr at room temperature. The resulting mixture was diluted with water (60 mL). The resulting mixture was extracted with ethyl acetate (3×50 mL). The combined organic layers were washed with water (3×50 mL), dried over anhydrous Na₂SO₄. After filtration, the filtrate was concentrated under reduced pressure. The residue was purified by silica gel column chromatography, eluted with EA to afford tert-butyl 4-[7-({8-fluoro-2-methylimidazo[1,2-a]pyridin-6-yl}carbamoyl)-2-(1-methoxy-1-oxopropan-2-yl) indazol-4-yl]piperazine-1-carboxylate (C255, 520 mg, 55%) as a solid. LCMS (ES, m/z): 580 [M+H]⁺

### Synthesis of Intermediate C256

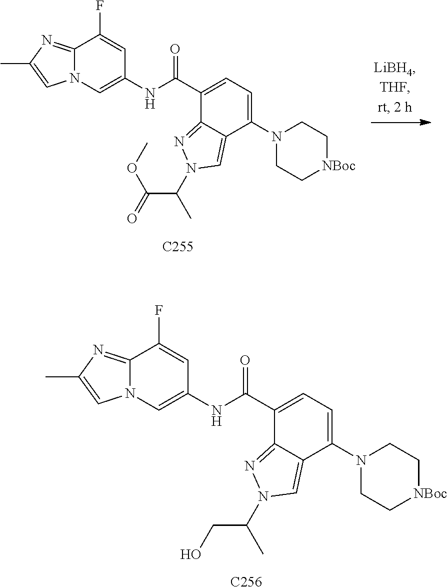

To a stirred mixture of tert-butyl 4-[7-({8-fluoro-2-methylimidazo[1,2-a]pyridin-6-yl}carbamoyl)-2-(1-methoxy-1-oxopropan-2-yl) indazol-4-yl]piperazine-1-carboxylate (520.0 mg, 0.897 mmol, 1.0 equiv) in THF (10 mL) was added LiBH₄ (58.6 mg, 2.691 mmol, 3.0 equiv) at 0° C. under nitrogen atmosphere. The resulting mixture was stirred for 1 hr at 0° C. under nitrogen atmosphere. The reaction was quenched with water/ice at 0° C. The resulting mixture was extracted with ethyl acetate (2×20 mL). The combined organic layers were washed with water (1×30 mL), dried over anhydrous Na₂SO₄. After filtration, the filtrate was concentrated under reduced pressure. The residue was purified by silica gel column chromatography, eluted with PE/EA (1:9) to afford tert-butyl 4-[7-({8-fluoro-2-methylimidazo[1,2-a]pyridin-6-yl}carbamoyl)-2-(1-hydroxypropan-2-yl) indazol-4-yl]piperazine-1-carboxylate (C256, 420 mg, 84%) as a solid. LCMS (ES, m/z): 552 [M+H]⁺

### Synthesis of Intermediate C257

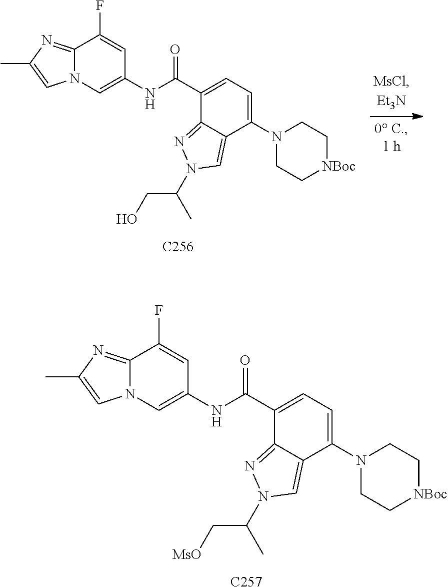

To a stirred mixture of tert-butyl 4-[7-({8-fluoro-2-methylimidazo[1,2-a]pyridin-6-yl}carbamoyl)-2-(1-hydroxypropan-2-yl) indazol-4-yl]piperazine-1-carboxylate (420.0 mg, 0.761 mmol, 1.0 equiv) and Et₃N (231.1 mg, 2.283 mmol, 3.0 equiv) in DCM (8 mL) was added MsCl (104.6 mg, 0.913 mmol, 1.2 equiv) dropwise at 0° C. under nitrogen atmosphere. The resulting mixture was stirred for 1 hr at 0° C. under nitrogen atmosphere. The reaction was quenched with water/ice at 0° C. The resulting mixture was extracted with CH₂Cl₂ (2×10 mL). The combined organic layers were washed with water (1×20 mL), dried over anhydrous Na₂SO₄. After filtration, the filtrate was concentrated under reduced pressure. The residue was purified by silica gel column chromatography, eluted with PE/EA (1:9) to afford tert-butyl 4-[7-({8-fluoro-2-methylimidazo[1,2-a]pyridin-6-yl}carbamoyl)-2-[1-(methanesulfonyloxy)propan-2-yl]indazol-4-yl]piperazine-1-carboxylate (C257, 450 mg, 93%) as a solid. LCMS (ES, m/z): 630 [M+H]⁺

### Synthesis of Intermediate C258

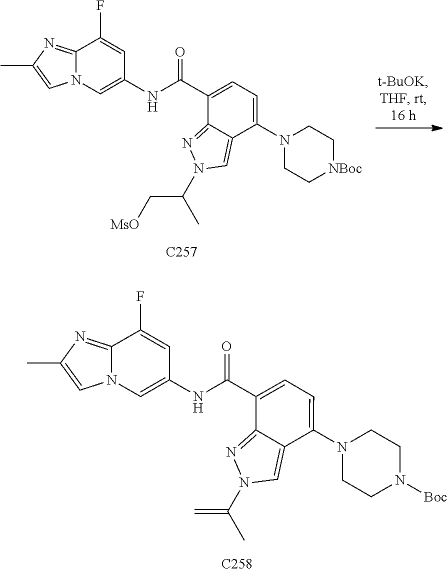

To a stirred mixture of tert-butyl 4-[7-({8-fluoro-2-methylimidazo[1,2-a]pyridin-6-yl}carbamoyl)-2-[1-(methanesulfonyloxy)propan-2-yl]indazol-4-yl]piperazine-1-carboxylate (450.0 mg, 0.715 mmol, 1.0 equiv) in THF (9 mL) was added tert-butoxypotassium (160.4 mg, 1.430 mmol, 2.0 equiv) at room temperature. The resulting mixture was stirred for 16 h at room temperature. The resulting mixture was diluted with water (20 mL). The resulting mixture was extracted with ethyl acetate (2×20 mL). The combined organic layers were washed with water (1×40 mL), dried over anhydrous Na₂SO₄. After filtration, the filtrate was concentrated under reduced pressure. The residue was purified by silica gel column chromatography, eluted with PE/EA (1:9) to afford tert-butyl 4-[7-({8-fluoro-2-methylimidazo[1,2-a]pyridin-6-yl}carbamoyl)-2-(prop-1-en-2-yl) indazol-4-yl]piperazine-1-carboxylate (C258, 210 mg, 55%) as a solid. LCMS (ES, m/z): 534 [M+H]⁺

### Synthesis of Compound 440

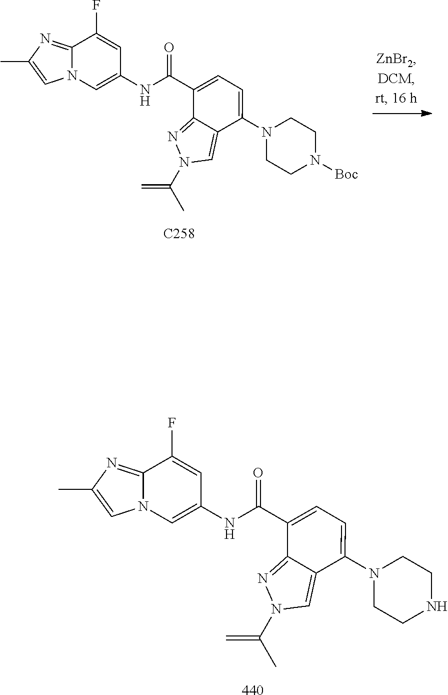

To a stirred mixture of tert-butyl 4-[7-({8-fluoro-2-methylimidazo[1,2-a]pyridin-6-yl}carbamoyl)-2-(prop-1-en-2-yl) indazol-4-yl]piperazine-1-carboxylate (100 mg, 0.187 mmol, 1.0 equiv) in DCM (2 mL) was added ZnBr₂ (422.1 mg, 1.870 mmol, 10.0 equiv) at room temperature. The resulting mixture was stirred for 16 hr at room temperature. The resulting mixture was concentrated under vacuum. The crude product was purified by Prep-HPLC (Condition 10, Gradient 9) to afford N-{8-fluoro-2-methylimidazo[1,2-a]pyridin-6-yl}-4-(piperazin-1-yl)-2-(prop-1-en-2-yl) indazole-7-carboxamide (Compound 440, 6 mg, 7%) as a solid. LCMS (ES, m/z): 434 [M+H]^{+ 1}H NMR (400 MHz, DMSO-d₆) δ 10.93 (s, 1H), 9.20 (d, J=1.6 Hz, 1H), 8.99 (s, 1H), 8.03 (d, J=8.1 Hz, 1H), 7.97-7.83 (m, 1H), 7.20 (dd, J=12.2, 1.7 Hz, 1H), 6.52 (d, J=8.2 Hz, 1H), 6.01 (s, 1H), 5.23 (s, 1H), 3.39 (t, J=5.0 Hz, 4H), 2.93 (t, J=4.9 Hz, 4H), 2.54 (s, 3H), 2.35 (s, 3H).

### Example 182: Synthesis of Compound 450

### Synthesis of Intermediate C259

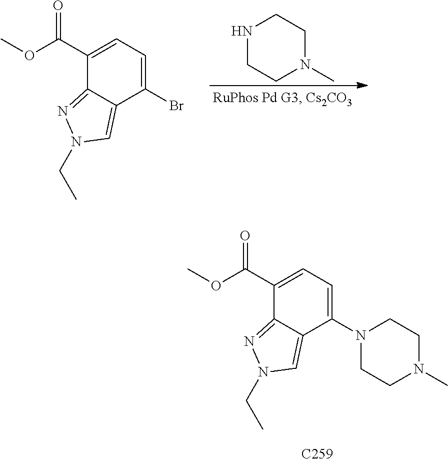

To a solution of methyl 4-bromo-2-ethylindazole-7-carboxylate (1 g, 3.532 mmol, 1 equiv) and 1-methylpiperazine (0.42 g, 4.238 mmol, 1.2 equiv) in dioxane (20 mL) were added Cs₂CO₃ (3.45 g, 10.596 mmol, 3.0 equiv), RuPhos (0.16 g, 0.353 mmol, 0.1 equiv) and RuPhos Palladacycle Gen.3 (0.3 g, 0.353 mmol, 0.1 equiv). After stirring for 1 hr at 90° C. under a nitrogen atmosphere, the resulting mixture was concentrated under reduced pressure. The residue was purified by reverse flash chromatography (Condition 3, Gradient 1) to afford methyl 2-ethyl-4-(4-methylpiperazin-1-yl) indazole-7-carboxylate (C259, 1 g, 93%) as a solid. LCMS (ES, m/z): 303 [M+H]⁺

### Synthesis of Intermediate C260

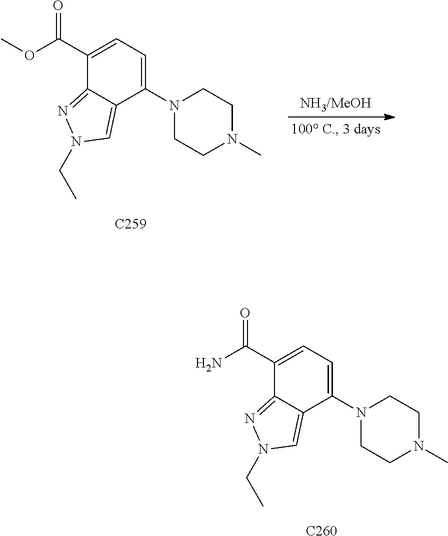

A solution of methyl 2-ethyl-4-(4-methylpiperazin-1-yl) indazole-7-carboxylate (200 mg, 0.661 mmol, 1 equiv) in 7 N NH₃(g) in MeOH (40 mL) was stirred for 3 days at 100° C. The resulting mixture was concentrated under reduced pressure. The crude product was used in the next step directly without further purification. LCMS (ES, m/z): 288 [M+H]⁺

### Synthesis of Intermediate C261

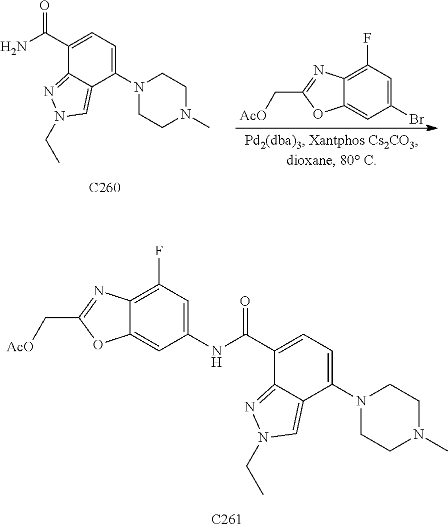 [SMILES: CCn1cc2c(N3CCN(C)CC3)ccc(C(N)=O)c2n1 ; CC(=O)OCc1nc2c(F)cc(Br)cc2o1 ; "C260" ; CCn1cc2c(N3CCN(C)CC3)ccc(C(=O)Nc3cc(F)c4nc(COC(C)=O)oc4c3)c2n1 ; "C261"]

To a solution of 2-ethyl-4-(4-methylpiperazin-1-yl) indazole-7-carboxamide (200 mg, 0.696 mmol, 1 equiv) and (6-bromo-4-fluoro-1,3-benzoxazol-2-yl)methyl acetate (240 mg, 0.833 mmol, 1.20 equiv) in dioxane (6 mL) were added Cs₂CO₃ (680 mg, 2.088 mmol, 3.0 equiv), XantPhos (81 mg, 0.139 mmol, 0.2 equiv) and Pd₂(dba)₃ (64 mg, 0.070 mmol, 0.1 equiv). After stirring for 2 h at 80° C. under a nitrogen atmosphere, the resulting mixture was concentrated under reduced pressure. The residue was purified by reverse flash chromatography (Condition 3, Gradient 1) to afford{6-[2-ethyl-4-(4-methylpiperazin-1-yl) indazole-7-amido]-4-fluoro-1,3-benzoxazol-2-yl}methyl acetate (C261, 100 mg, 29%) as a solid. LCMS (ES, m/z): 495 [M+H]⁺

### Synthesis of Compound 450

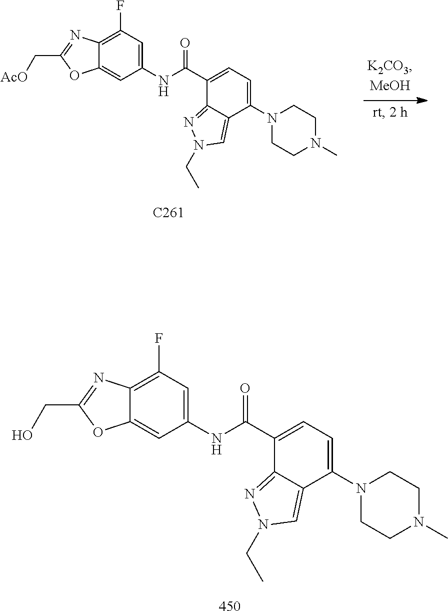 [SMILES: CCn1cc2c(N3CCN(C)CC3)ccc(C(=O)Nc3cc(F)c4nc(COC(C)=O)oc4c3)c2n1 ; "C261" ; CCn1cc2c(N3CCN(C)CC3)ccc(C(=O)Nc3cc(F)c4nc(CO)oc4c3)c2n1 ; "450"]

A solution of {6-[2-ethyl-4-(4-methylpiperazin-1-yl) indazole-7-amido]-4-fluoro-1,3-benzoxazol-2-yl}methyl acetate (100 mg, 0.202 mmol, 1 equiv) and K₂CO₃ (167 mg, 1.212 mmol, 6.0 equiv) in methanol (3 mL) was stirred for 2 hr at room temperature. The resulting mixture was concentrated under reduced pressure. The residue was purified by reverse flash chromatography (Condition 3, Gradient 5) to afford 2-ethyl-N-[4-fluoro-2-(hydroxymethyl)-1,3-benzoxazol-6-yl]-4-(4-methylpiperazin-1-yl) indazole-7-carboxamide (Compound 450, 24.8 mg, 27%) as a solid. LCMS (ES, m/z): 453 [M+H]^{+ 1}H NMR (300 MHz, DMSO-d₆) δ 11.53 (s, 1H), 8.85 (s, 1H), 8.18 (d, J=1.6 Hz, 1H), 8.01 (d, J=8.1 Hz, 1H), 7.65 (dd, J=12.1, 1.7 Hz, 1H), 6.52 (d, J=8.2 Hz, 1H), 5.93 (s, 1H), 4.72 (s, 2H), 4.61 (q, J=7.2 Hz, 2H), 3.45 (t, J=4.9 Hz, 4H), 2.55 (d, J=4.9 Hz, 4H), 2.28 (s, 3H), 1.63 (t, J=7.3 Hz, 3H).

### Example 183: Synthesis of Compound 451

### Synthesis of Intermediate C262

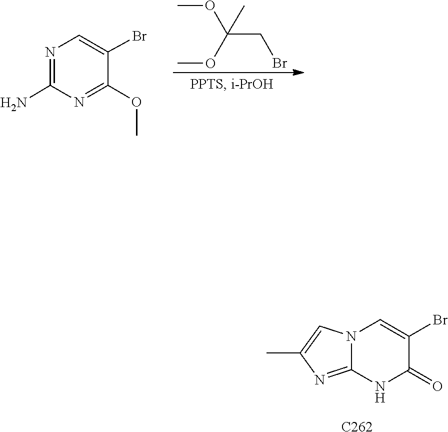

A solution of 5-bromo-4-methoxypyrimidin-2-amine (20 g, 98.026 mmol, 1 equiv) in isopropanol (480 mL) was treated with 1-bromo-2,2-dimethoxypropane (26.91 g, 147.039 mmol, 1.5 equiv) and PPTS (2.46 g, 9.803 mmol, 0.1 equiv) for 48 hours at 80° C. The precipitated solids were collected by filtration and washed with isopropanol (50 mL). To afford 6-bromo-2-methyl-8H-imidazo[1,2-a]pyrimidin-7-one (17.5 g, 69.32%) as a solid. LCMS (ES, m/z): 228 [M+H]⁺

### Synthesis of Intermediate C263

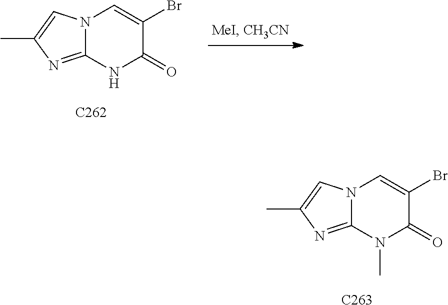

A solution of 6-bromo-2-methyl-8H-imidazo[1,2-a]pyrimidin-7-one (2 g, 8.770 mmol, 1 equiv) in MeCN (40 mL) was treated with K₂CO₃ (3.64 g, 26.310 mmol, 3 equiv), CH₃I (1.87 g, 13.155 mmol, 1.5 equiv) for 8 hours at 50° C. The resulting mixture was diluted with MeOH (100 mL). The residue was purified by silica gel column chromatography, eluted with DCM:MeOH (20/1) to afford 6-bromo-2,8-dimethylimidazo[1,2-a]pyrimidin-7-one (C263, 800 mg, 37%) as a solid. LCMS (ES, m/z): 242 [M+H]^{+ 1}H NMR (300 MHz, DMSO-d6) δ 8.67 (d, J=9.3 Hz, 1H), 7.10 (dd, J=10.7, 1.4 Hz, 1H), 4.95 (s, 1H), 3.37 (d, J=14.2 Hz, 13H), 2.29-2.21 (m, 3H), 2.10 (d, J=1.3 Hz, 1H)

### Synthesis of Intermediate C264

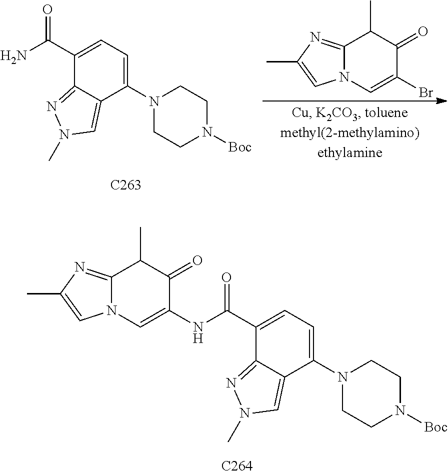

A solution of tert-butyl 4-(7-carbamoyl-2-methylindazol-4-yl)piperazine-1-carboxylate (445 mg, 1.239 mmol, 1 equiv) in toluene (10 mL) as treated with 6-bromo-2,8-dimethylimidazo[1,2-a]pyrimidin-7-one (300 mg, 1.239 mmol, 1.00 equiv), methyl[2-(methylamino)ethyl]amine (218 mg, 2.478 mmol, 2 equiv), Cu (78 mg, 1.239 mmol, 1 equiv), K₂CO₃ (342 mg, 2.478 mmol, 2 equiv) for 8 hours at 100° C. under nitrogen atmosphere. The resulting mixture was filtered and the filter cake was washed with MeOH (100 mL). The filtrate was concentrated under reduced pressure. The residue was purified by silica gel column chromatography, eluted with MeOH/DCM (1/10) to afford tert-butyl 4-[7-({2,8-dimethyl-7-oxoimidazo[1,2-a]pyrimidin-6-yl}carbamoyl)-2-methylindazol-4-yl]piperazine-1-carboxylate (100 mg, 15%) as a solid. LCMS (ES, m/z): 521 [M+H]⁺

### Synthesis of Compound 451

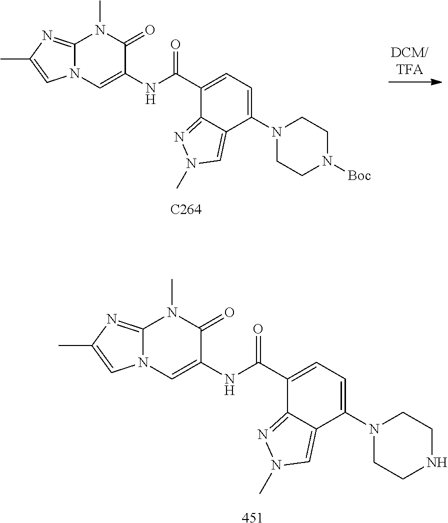

A solution of tert-butyl 4-[7-({2,8-dimethyl-7-oxoimidazo[1,2-a]pyrimidin-6-yl}carbamoyl)-2-methylindazol-4-yl]piperazine-1-carboxylate (100 mg, 0.192 mmol, 1 equiv) in DCM (20 mL) was treated with TFA (2 mL) for 1 hour at 20° C. The residue was purified by reverse flash chromatography (Condition 2, Gradient 1) to afford N-{2,8-dimethyl-7-oxoimidazo[1,2-a]pyrimidin-6-yl}-2-methyl-4-(piperazin-1-yl) indazole-7-carboxamide (Compound 451, 10 mg, 12%) as a solid. LCMS (ES, m/z): 421 [M+H]^{+ 1}H NMR (300 MHz, DMSO-d6) δ 11.47 (s, 1H), 9.25 (s, 1H), 8.75 (s, 1H), 7.97 (d, J=8.1 Hz, 1H), 7.31 (d, J=1.5 Hz, 1H), 6.47 (d, J=8.2 Hz, 1H), 4.24 (s, 3H), 3.46 (s, 3H), 3.35 (s, 15H), 2.92 (d, J=6.0 Hz, 3H), 2.26 (d, J=1.3 Hz, 3H)

### Example 184: Synthesis of Compound 453

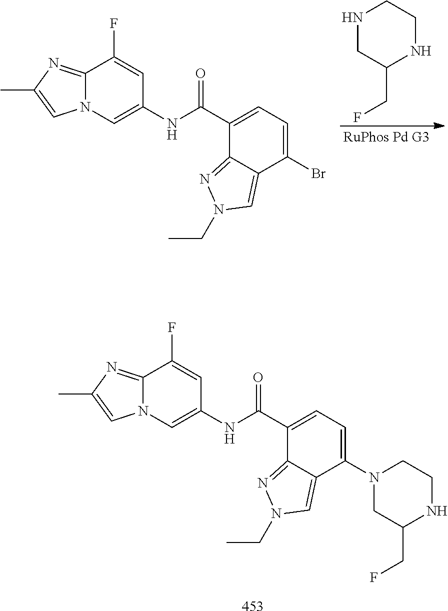

To a solution of 4-bromo-2-ethyl-N-{8-fluoro-2-methylimidazo[1,2-a]pyridin-6-yl}indazole-7-carboxamide (200 mg, 0.480 mmol, 1 equiv) and 2-(fluoromethyl)piperazine (125 mg, 1.056 mmol, 2.2 equiv) in dioxane (5 mL) were added Cs₂CO₃ (469.64 mg, 1.440 mmol, 3.0 equiv), RuPhos (45 mg, 0.096 mmol, 0.2 equiv) and RuPhos Palladacycle Gen.3 (41 mg, 0.048 mmol, 0.1 equiv). After stirring for 3 hr at 90° C. under a nitrogen atmosphere, the resulting mixture was concentrated under reduced pressure. The residue was purified by reverse flash chromatography (condition 6, Gradient 1) to afford 2-ethyl-N-{8-fluoro-2-methylimidazo[1,2-a]pyridin-6-yl}-4-[3-(fluoromethyl)piperazin-1-yl]indazole-7-carboxamide (Compound 453, 54 mg, 24%) as a solid. LCMS (ES, m/z): 454 [M+H]^{+ 1}H NMR (300 MHz) 9.15 (d, J=13.9 Hz, 2H), 8.55 (d, J=8.4 Hz, 1H), 8.06 (d, J=10.0 Hz, 1H), 7.97 (s, 1H), 7.13 (d, J=8.5 Hz, 1H), 5.01-4.99 (m, 4H), 4.31 (d, J=12.7 Hz, 3H), 4.03-3.87 (m, 4H), 2.75 (d, J=1.1 Hz, 3H), 1.92 (t, J=7.2 Hz, 3H).

### Example 185: Synthesis of Compound 454

### Synthesis of Intermediate C265

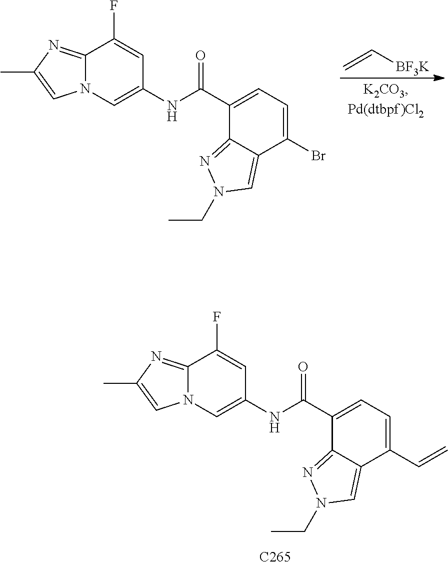

To a solution of 4-bromo-2-ethyl-N-{8-fluoro-2-methylimidazo[1,2-a]pyridin-6-yl}indazole-7-carboxamide (500 mg, 1.201 mmol, 1 equiv) and potassium ethenyltrifluoroboranuide (193 mg, 1.441 mmol, 1.2 equiv) in dioxane (8 mL) and H₂O (2 mL) were added K₂CO₃ (498 mg, 3.603 mmol, 3.0 equiv) and Pd(dtbpf)Cl₂ (78 mg, 0.120 mmol, 0.1 equiv). After stirring for 1 hr at 80° C. under a nitrogen atmosphere, the resulting mixture was concentrated under reduced pressure. The residue was purified by reverse flash chromatography (Condition 5, Gradient 2) to afford 4-ethenyl-2-ethyl-N-{8-fluoro-2-methylimidazo[1,2-a]pyridin-6-yl}indazole-7-carboxamide (C265, 400 mg, 91%) as a solid. LCMS (ES, m/z): 364 [M+H]⁺

### Synthesis of Intermediate C266

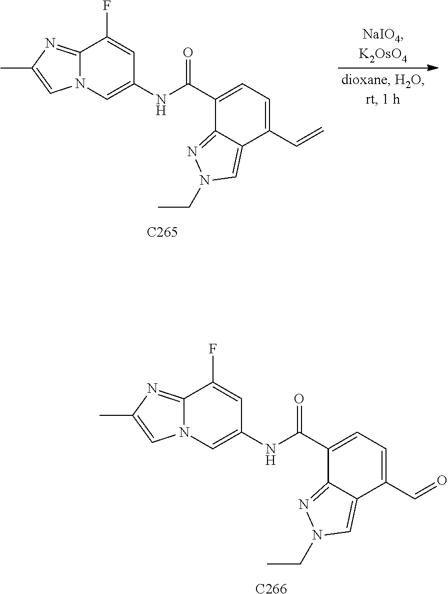

To a solution of 4-ethenyl-2-ethyl-N-{8-fluoro-2-methylimidazo[1,2-a]pyridin-6-yl}indazole-7-carboxamide (200 mg, 0.550 mmol, 1 equiv) and NaIO₄ (235.4 mg, 1.100 mmol, 2.0 equiv) in dioxane (6 mL) and H₂O (2 mL) were added K₂OsO₄·2H₂O (20.3 mg, 0.055 mmol, 0.1 equiv). After stirring for 1 hr at room temperature under a nitrogen atmosphere, the resulting mixture was concentrated under reduced pressure. The residue was purified by silica gel column chromatography, eluted with PE:EA (4:1) to afford 2-ethyl-N-{8-fluoro-2-methylimidazo[1,2-a]pyridin-6-yl}-4-formylindazole-7-carboxamide (C266, 40 mg, 19%) as a solid. LCMS (ES, m/z): 366 [M+H]⁺

### Synthesis of Intermediate C267

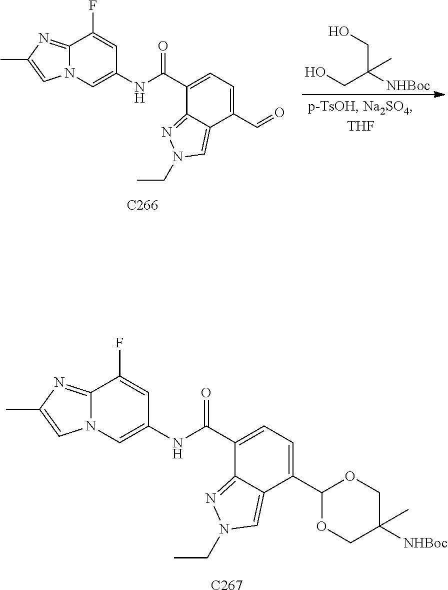

To a stirred solution of 2-ethyl-N-{8-fluoro-2-methylimidazo[1,2-a]pyridin-6-yl}-4-formylindazole-7-carboxamide (40 mg, 0.109 mmol, 1 equiv) and tert-butyl N-(1,3-dihydroxy-2-methylpropan-2-yl)carbamate (80 mg, 0.390 mmol, 3.56 equiv) in DCM (2 mL) and THF (0.2 mL) were addedp-TsOH (56 mg, 0.325 mmol, 2.97 equiv) and Na₂SO₄ (80 mg, 0.563 mmol, 5.14 equiv) in portions at room temperature. The resulting mixture was stirred for 12 h at room temperature. The resulting mixture was diluted with H₂O (5 mL). The resulting mixture was extracted with EA (3×5 mL). The combined organic layers were washed with brine (1×5 mL), dried over anhydrous Na₂SO₄. After filtration, the filtrate was concentrated under reduced pressure. The crude product was used in the next step directly without further purification. LCMS (ES, m/z): 553 [M+H]⁺

### Synthesis of Compound 454

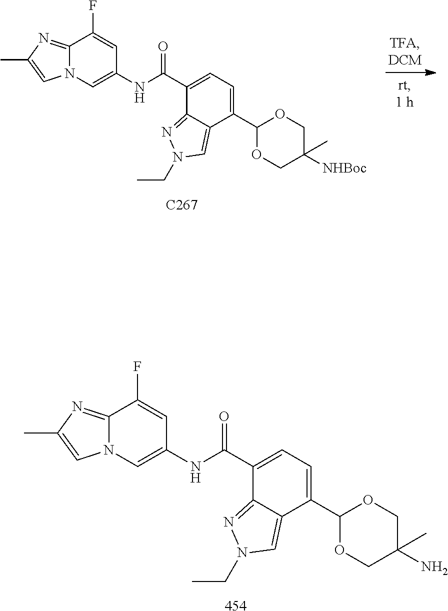

A solution of tert-butyl N-{2-[2-ethyl-7-({8-fluoro-2-methylimidazo[1,2-a]pyridin-6-yl}carbamoyl) indazol-4-yl]-5-methyl-1,3-dioxan-5-yl}carbamate (20 mg, 0.036 mmol, 1 equiv) and TFA (0.2 mL) in DCM (3 mL) was stirred for 1 hr at room temperature. The mixture was basified to pH 8 with NH₃ in MeOH. The resulting mixture was concentrated under reduced pressure. The residue was purified by reverse flash chromatography (Condition 6, Gradient 1) to afford 4-(5-amino-5-methyl-1,3-dioxan-2-yl)-2-ethyl-N-{8-fluoro-2-methylimidazo[1,2-a]pyridin-6-yl}indazole-7-carboxamide (Compound 454, 5 mg, 30%) as a solid. LCMS (ES, m/z): 453 [M+H]^{+ 1}H NMR (300 MHz, Methanol-d₄) δ 9.14 (d, J=1.7 Hz, 1H), 8.58 (s, 1H), 8.17 (d, J=7.4 Hz, 1H), 7.73 (dd, J=3.1, 1.0 Hz, 1H), 7.39 (dd, J=7.4, 0.8 Hz, 1H), 7.23 (dd, J=11.7, 1.7 Hz, 1H), 5.81 (s, 1H), 4.68 (q, J=7.3 Hz, 2H), 4.00 (d, J=11.0 Hz, 2H), 3.90 (d, J=10.9 Hz, 2H), 2.44 (d, J=0.9 Hz, 3H), 1.73 (t, J=7.3 Hz, 3H), 1.05 (s, 3H).

### Example 186: Synthesis of Compounds 456 and 484

### Synthesis of Intermediate C268

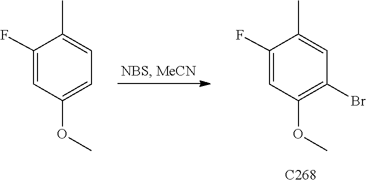

A solution of 2-fluoro-4-methoxy-1-methylbenzene (10 g, 71.349 mmol, 1 equiv) and NBS (13.3 g, 74.916 mmol, 1.05 equiv) in MeCN (200 mL) was stirred for 4 h at room temperature. The reaction was quenched with water (200 mL) at room temperature. The resulting mixture was extracted with ethyl acetate (3×200 mL). The combined organic layers were dried over anhydrous Na₂SO₄. After filtration, the filtrate was concentrated under reduced pressure. The residue was purified by silica gel column chromatography, eluted with PE/EA (5:1) to afford 1-bromo-4-fluoro-2-methoxy-5-methylbenzene (C268, 12 g, 76%) as a solid. ¹H NMR (300 MHz, DMSO-d6) δ 7.50 (dd, J=8.3, 0.8 Hz, 1H), 7.01 (d, J=11.6 Hz, 1H), 3.83 (s, 3H), 2.15 (dd, J=2.0, 0.7 Hz, 3H).

### Synthesis of Intermediate C269

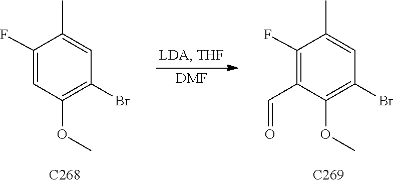

To a stirred mixture of 1-bromo-4-fluoro-2-methoxy-5-methylbenzene (12 g, 54.781 mmol, 1 equiv) in THF (240 mL) was added LDA (in 2M THF) (36 mL, 71.1 mmol, 1.3 equiv) dropwise at −78° C. under nitrogen atmosphere. The resulting mixture was stirred for 1 hr at −78° C. under nitrogen atmosphere. Then the mixture was added DMF (20.02 g, 273.905 mmol, 5 equiv) dropwise at −78° C. under nitrogen atmosphere and stirred at room temperature for 16 hr. The reaction was quenched with sat. NH₄Cl (aq.) (100 mL) at room temperature. The resulting mixture was extracted with ethyl acetate (3×100 mL). The combined organic layers dried over anhydrous Na₂SO₄. After filtration, the filtrate was concentrated under reduced pressure. The residue was purified by silica gel column chromatography, eluted with PE/EA (5:1) to afford 3-bromo-6-fluoro-2-methoxy-5-methylbenzaldehyde (C269, 9 g, 66%) as an oil. ¹H NMR (300 MHz, DMSO-d6) δ 10.21 (d, J=1.1 Hz, 1H), 7.95 (m, 1H), 3.87 (s, 3H), 2.23 (dd, J=2.4, 0.8 Hz, 4H).

### Synthesis of Intermediate C270

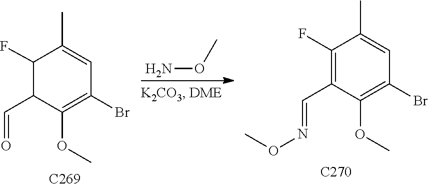

A mixture of 3-bromo-6-fluoro-2-methoxy-5-methylbenzaldehyde (8 g, 32.380 mmol, 1 equiv), O-methylhydroxylamine (1.68 g, 35.618 mmol, 1.1 equiv) and K₂CO₃ (6.71 g, 48.570 mmol, 1.5 equiv) in DME (80 mL) was stirred for 4 hr at 60° C. The reaction was quenched with water (100 mL) at room temperature. The resulting mixture was extracted with ethyl acetate (3×100 mL). The combined organic layers were dried over anhydrous Na₂SO₄. After filtration, the filtrate was concentrated under reduced pressure to afford (E)-[(3-bromo-6-fluoro-2-methoxy-5-methylphenyl)methylidene](methoxy)amine (C270, 7 g, 78%) as a solid. LCMS (ES, m/z): 276 [M+H]⁺

### Synthesis of Intermediate C271

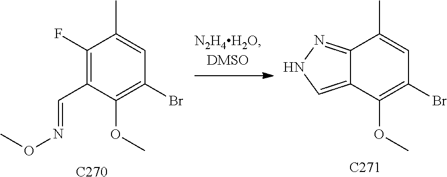

A mixture of 3-bromo-6-fluoro-2-methoxy-5-methylbenzaldehyde O-methyl (6 g, 21.82 mmol, 1 equiv) in DMSO (70 mL) and N₂H₄·H₂O (70 mL) was stirred for 4 h at 140° C. The reaction was quenched with water (100 mL) at room temperature. The resulting mixture was extracted with ethyl acetate (3×100 mL). The combined organic layers were dried over anhydrous Na₂SO₄. After filtration, the filtrate was concentrated under reduced pressure. The residue was purified by silica gel column chromatography, eluted with PE/EA (1:1) to afford 5-bromo-4-methoxy-7-methyl-2H-indazole (C271, 1.0 g, 19%) as a solid. LCMS (ES, m/z): 241 [M+H]⁺

### Synthesis of Intermediate C272

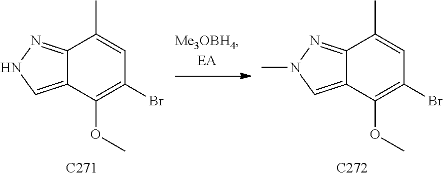

A mixture of 5-bromo-4-methoxy-7-methyl-2H-indazole (300 mg, 1.244 mmol, 1 equiv) and tetrafluoroboranuide; trimethyloxidanium (920.3 mg, 6.220 mmol, 5 equiv) in EA (3 mL) was stirred for 2 hr at 25° C. under N₂ atmosphere. The resulting mixture was concentrated under vacuum. The residue was purified by silica gel column chromatography, eluted with PE/EA to afford 5-bromo-4-methoxy-2,7-dimethylindazole (C272, 270 mg, 85%) as a solid. LCMS (ES, m/z): 255 [M+H]⁺

### Synthesis of Intermediate C273

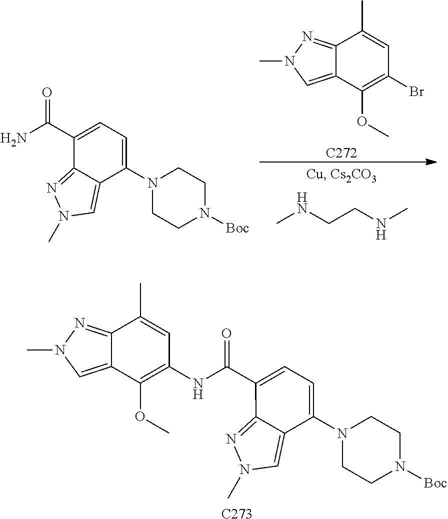

A mixture of tert-butyl 4-(7-carbamoyl-2-methylindazol-4-yl)piperazine-1-carboxylate (200 mg, 0.556 mmol, 1 equiv) and 5-bromo-4-methoxy-2,7-dimethylindazole (170.4 mg, 0.667 mmol, 1.2 equiv), methyl[2-(methylamino)ethyl]amine (24.5 mg, 0.278 mmol, 0.5 equiv), Cu (17.7 mg, 0.278 mmol, 0.5 equiv), K₂CO₃ (230.7 mg, 1.668 mmol, 3 equiv) in xylene (2 mL) was stirred for 16 h at 120° C. under N₂ atmosphere. The residue was purified by silica gel column chromatography, eluted with DCM/MeOH to afford tert-butyl 4-{7-[(4-methoxy-2,7-dimethylindazol-5-yl)carbamoyl]-2-methylindazol-4-yl}piperazine-1-carboxylate (C273, 70 mg, 23%) as a solid. LCMS (ES, m/z): 533 [M+H]⁺

### Synthesis of Compound 456

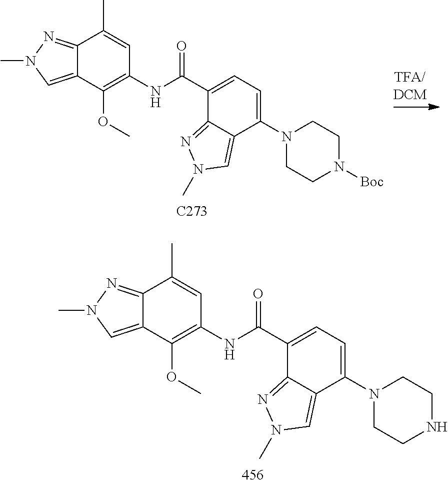

A mixture of tert-butyl 4-{7-[(4-methoxy-2,7-dimethylindazol-5-yl)carbamoyl]-2-methylindazol-4-yl}piperazine-1-carboxylate (70 mg, 0.131 mmol, 1 equiv) and TFA (0.2 mL, 2.693 mmol, 20.53 equiv) in DCM (1 mL) was stirred for 1 hr at 25° C. under N₂ atmosphere. The resulting mixture was concentrated under reduced pressure. The residue was purified by reverse flash chromatography (Condition 10, Gradient 1) to afford N-(4-hydroxy-2,7-dimethylindazol-5-yl)-2-methyl-4-(piperazin-1-yl) indazole-7-carboxamide (Compound 456, 5 mg) as a solid. LCMS (ES, m/z): 434[M+H]^{+ 1}H NMR (300 MHz, DMSO-d6) δ 11.30 (s, 1H), 8.75 (s, 1H), 8.53 (s, 1H), 8.28 (d, J=1.3 Hz, 1H), 7.99 (d, J=8.0 Hz, 1H), 6.48 (d, J=8.1 Hz, 1H), 4.27 (s, 3H), 4.15 (d, J=15.7 Hz, 6H), 3.30 (s, 1H), 2.92 (t, J=4.9 Hz, 4H), 2.47 (d, J=1.1 Hz, 3H).

### Synthesis of Compound 484

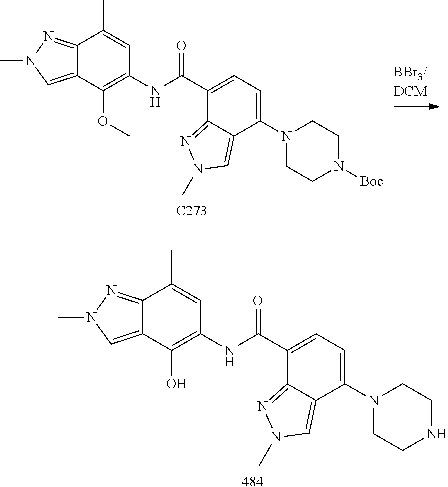

A mixture of tert-butyl 4-{7-[(4-methoxy-2,7-dimethylindazol-5-yl)carbamoyl]-2-methylindazol-4-yl}piperazine-1-carboxylate (45 mg, 0.084 mmol, 1 equiv) and BBr₃ (0.5 mL, 5.289 mmol, 62.72 equiv) in DCM (0.5 mL) was stirred for 16 h at 25° C. under N₂ atmosphere. The product was precipitated by the addition of MeOH. The precipitated solids were collected by filtration and washed with MeOH. The residue was purified by reverse flash chromatography (Condition 10, Gradient 1) to afford N-(4-hydroxy-2,7-dimethylindazol-5-yl)-2-methyl-4-(piperazin-1-yl) indazole-7-carboxamide (Compound 484, 11 mg, 31%) as a solid. LCMS (ES, m/z): 420[M+H]^{+ 1}H NMR (300 MHz, DMSO-d6) δ 11.10 (s, 1H), 10.34 (s, 1H), 8.91 (d, J=9.3 Hz, 3H), 8.32 (s, 1H), 8.05 (d, J=8.0 Hz, 1H), 7.45 (d, J=1.2 Hz, 1H), 6.62 (d, J=8.1 Hz, 1H), 4.29 (s, 3H), 4.16 (s, 3H), 3.36 (s, 4H), 2.43 (d, J=1.0 Hz, 3H).

### Example 187: Synthesis of Compound 457

### Synthesis of Intermediate C274

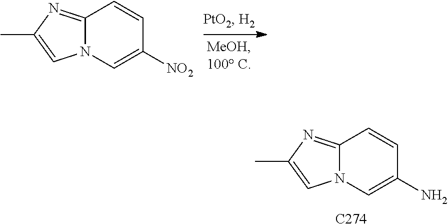

A solution of 2-methyl-6-nitroimidazo[1,2-a]pyridine (150 mg, 0.847 mmol, 1 equiv) in MeOH (20 mL) was treated with PtO₂ (75 mg, 0.330 mmol, 0.39 equiv) for 8 hr at 80° C. under 20 atm hydrogen atmosphere. The resulting mixture was filtered and the filter cake was washed with methanol. The filtrate was concentrated under reduced pressure. The crude product 2-methyl-5H,6H,7H,8H-imidazo[1,2-a]pyridin-6-amine (C274, 146 mg, 66%) was used in the next step directly without further purification. LCMS (ES, m/z): 152[M+H]⁺

1. Synthesis of Intermediate c275

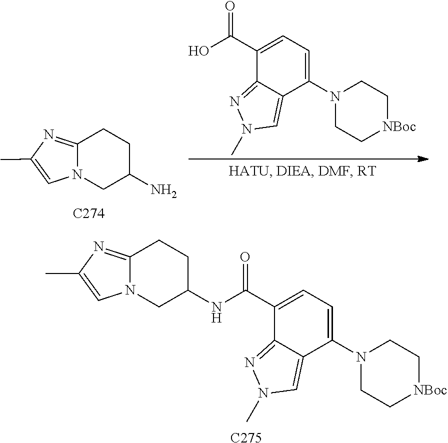

A solution of 4-[4-(tert-butoxycarbonyl)piperazin-1-yl]-2-methylindazole-7-carboxylic acid (100 mg, 0.277 mmol, 1.00 equiv) in DMF (3 mL) was treated with 2-methyl-5H,6H,7H,8H-imidazo[1,2-a]pyridin-6-amine (100 mg, 0.661 mmol, 2.38 equiv), HATU (126 mg, 0.332 mmol, 1.2 equiv), DIEA (107 mg, 0.831 mmol, 3 equiv) for 8 hr at 20° C. The residue was purified by reverse flash chromatography (Condition 1, Gradient 1) to afford tert-butyl 4-[2-methyl-7-({2-methyl-5H,6H,7H,8H-imidazo[1,2-a]pyridin-6-yl}carbamoyl) indazol-4-yl]piperazine-1-carboxylate (C275, 60 mg, 43%) as a solid. LCMS (ES, m/z): 494 [M+H]⁺

### Synthesis of Compound 457

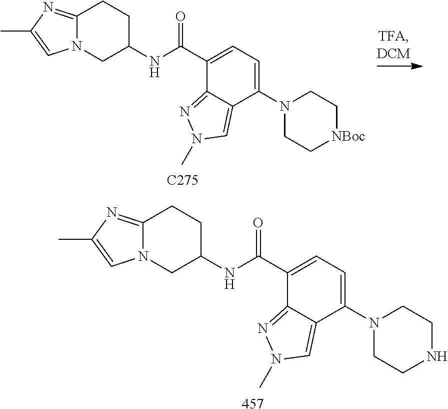

A solution of tert-butyl 4-[2-methyl-7-({2-methyl-5H,6H,7H,8H-imidazo[1,2-a]pyridin-6-yl}carbamoyl) indazol-4-yl]piperazine-1-carboxylate (50 mg, 0.101 mmol, 1 equiv) in DCM (10 mL) was treated with TFA (1 mL) for 1 hr at 0° C. The resulting mixture was diluted with water and concentrated under reduced pressure to afford 2-methyl-N-{2-methyl-5H,6H,7H,8H-imidazo[1,2-a]pyridin-6-yl}-4-(piperazin-1-yl) indazole-7-carboxamide (Compound 457, 30 mg, 75%) as a solid. LCMS (ES, m/z): 394 [M+H]^{+ 1}H NMR (300 MHz, DMSO-d6) δ 14.17 (s, 1H), 9.19 (d, J=7.0 Hz, 2H), 8.93 (s, 2H), 8.82 (s, 2H), 7.92 (d, J=7.9 Hz, 2H), 7.31 (d, J=1.3 Hz, 2H), 6.55 (d, J=8.0 Hz, 2H), 4.62 (s, 2H), 4.41 (dd, J=12.9, 4.4 Hz, 2H), 4.15 (s, 8H), 3.30-3.06 (m, 6H), 2.30-2.13 (m, 10H)

### Example 188: Synthesis of Compound 458

### Synthesis of Intermediate C276

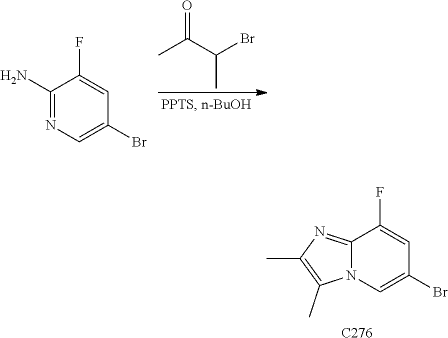

To a stirred solution of 5-bromo-3-fluoropyridin-2-amine (2 g, 10.471 mmol, 1 equiv) and 3-bromo-2-butanone (1.58 g, 10.471 mmol, 1 equiv) in n-BuOH (1 mL) was added pyridinium p-toluenesulfonate (263.14 mg, 1.047 mmol, 0.1 equiv) at room temperature. The resulting mixture was stirred for 72 hr at 120° C. The resulting mixture was concentrated under vacuum. The residue was purified by reverse flash (Condition 7, Gradient 1) to afford 6-bromo-8-fluoro-2,3-dimethylimidazo[1,2-a]pyridine (C276, 300 mg, 11%) as a solid. LCMS (ES, m/z): 243 [M+H]⁺

### Synthesis of Intermediate C277

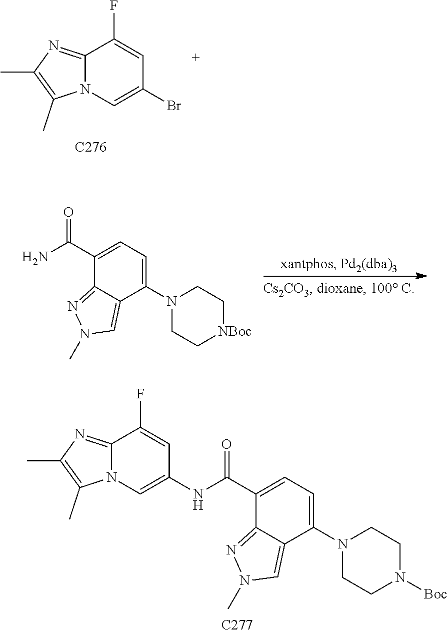

To a solution of tert-butyl 4-(7-carbamoyl-2-methylindazol-4-yl)piperazine-1-carboxylate (200 mg, 0.556 mmol, 1 equiv) and 6-bromo-8-fluoro-2,3-dimethylimidazo[1,2-a]pyridine (162.31 mg, 0.667 mmol, 1.2 equiv) in dioxane (10 mL) were added Pd₂(dba)₃ (50.95 mg, 0.056 mmol, 0.1 equiv), XantPhos (32.20 mg, 0.056 mmol, 0.1 equiv) and Cs₂CO₃ (543.89 mg, 1.668 mmol, 3 equiv). After stirring for 3 hr at 100° C. under a nitrogen atmosphere, the resulting mixture was concentrated under reduced pressure. The residue was purified by silica gel column chromatography, eluted with DCM:MeOH (10:1) to afford tert-butyl 4-[7-({8-fluoro-2,3-dimethylimidazo[1,2-a]pyridin-6-yl}carbamoyl)-2-methylindazol-4-yl]piperazine-1-carboxylate (C277, 200 mg, 68%) as a solid. LCMS (ES, m/z): 522 [M+H]⁺

### Synthesis of Compound 458

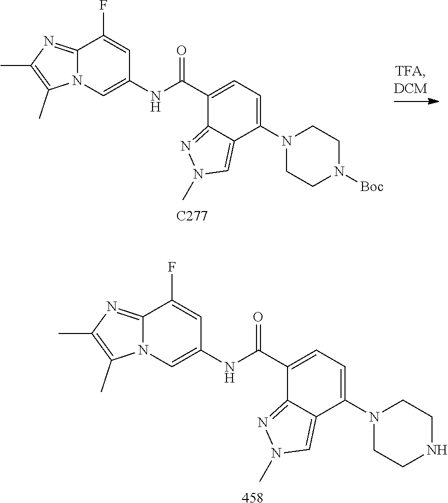

A solution of tert-butyl 4-[7-({8-fluoro-2,3-dimethylimidazo[1,2-a]pyridin-6-yl}carbamoyl)-2-methylindazol-4-yl]piperazine-1-carboxylate (110 mg, 0.211 mmol, 1 equiv) in DCM (4 mL) was added TFA (1 mL) at room temperature. The resulting mixture was stirred for 2 hr at room temperature. The resulting mixture was concentrated under reduced pressure. The residue was purified by reverse flash chromatography (Condition 7, Gradient 2) to afford N-{8-fluoro-2,3-dimethylimidazo[1,2-a]pyridin-6-yl}-2-methyl-4-(piperazin-1-yl) indazole-7-carboxamide (Compound 458, 13 mg, 14%) as a solid. LCMS (ES, m/z): 422 [M+H]^{+ 1}H NMR (400 MHz, Methanol-d₄) δ 11.12 (s, 1H), 8.91 (d, J=1.6 Hz, 1H), 8.80 (s, 1H), 7.98 (d, J=8.1 Hz, 1H), 7.32 (dd, J=12.4, 1.7 Hz, 1H), 6.49 (d, J=8.2 Hz, 1H), 4.30 (s, 3H), 3.36 (d, J=5.9 Hz, 4H), 2.92 (t, J=5.0 Hz, 4H), 2.42 (s, 3H), 2.34 (s, 3H).

### Example 189: Synthesis of Compound 459

### Synthesis of Intermediate C278

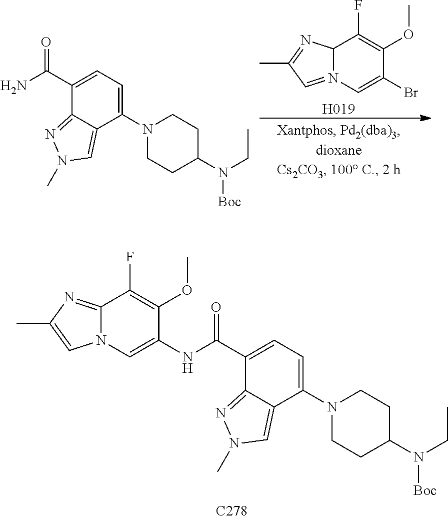

To a solution of tert-butyl N-[1-(7-carbamoyl-2-methylindazol-4-yl) piperidin-4-yl]-N-ethylcarbamate (150.0 mg, 0.374 mmol, 1.0 equiv) and 6-bromo-8-fluoro-7-methoxy-2-methylimidazo[1,2-a] pyridine (116.1 mg, 0.449 mmol, 1.2 equiv) in dioxane (4 mL) were added Cs₂CO₃ (305.2 mg, 0.935 mmol, 2.5 equiv) and Xantphos (43.2 mg, 0.075 mmol, 0.2 equiv), Pd₂(dba)₃ (34.2 mg, 0.037 mmol, 0.1 equiv). After stirring for 2 h at 100° C. under a nitrogen atmosphere, the resulting mixture was concentrated under reduced pressure. The residue was purified by silica gel column chromatography, eluted with CH₂Cl₂/MeOH (20:1) to afford tert-butyl N-ethyl-N-{1-[7-({8-fluoro-7-methoxy-2-methylimidazo[1,2-a] pyridin-6-yl}carbamoyl)-2-methylindazol-4-yl] piperidin-4-yl} carbamate (C278, 130 mg, 48%) as a solid. LCMS (ES, m/z): 580 [M+H]⁺

### Synthesis of Intermediate Compound 459

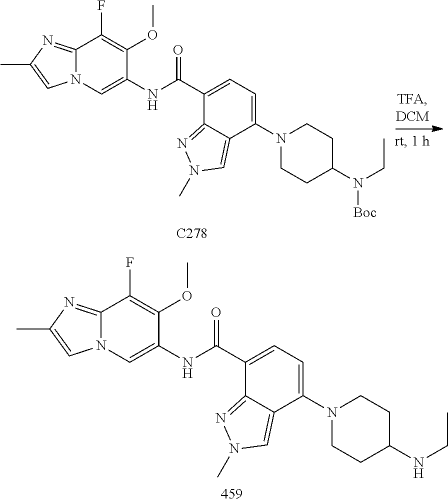

Into a 40 mL vial were added tert-butyl N-ethyl-N-{1-[7-({8-fluoro-7-methoxy-2-methylimidazo[1,2-a]pyridin-6-yl}carbamoyl)-2-methylindazol-4-yl]piperidin-4-yl}carbamate (90.0 mg, 0.155 mmol, 1.0 equiv), DCM (2 mL) and TFA (0.5 mL) at room temperature. The resulting mixture was stirred for 1 hr at room temperature. The resulting mixture was concentrated under reduced pressure. The crude product was purified by Prep-HPLC (Condition 13, Gradient 1) to afford 4-[4-(ethylamino)piperidin-1-yl]-N-{8-fluoro-7-methoxy-2-methylimidazo [1,2-a]pyridin-6-yl}-2-methylindazole-7-carboxamide (Compound 459, 8.0 mg, 11%) as a solid. LCMS (ES, m/z): 480 [M+H]^{+ 1}H NMR (400 MHz, Chloroform-d) δ 11.48 (s, 1H), 9.47 (d, J=1.3 Hz, 1H), 8.21 (d, J=8.0 Hz, 1H), 8.05 (s, 1H), 7.30 (d, J=3.0 Hz, 1H), 6.52 (d, J=8.1 Hz, 1H), 4.32 (s, 3H), 4.27 (d, J=2.3 Hz, 3H), 3.90 (d, J=12.7 Hz, 2H), 3.03 (t, J=11.8 Hz, 2H), 2.84 (q, J=7.0 Hz, 3H), 2.61-2.35 (m, 3H), 2.15 (d, J=12.5 Hz, 2H), 1.76-1.68 (m, 2H), 1.24 (t, J=7.1 Hz, 3H).

### Example 190: Synthesis of Compound 460

### Synthesis of Intermediate C279

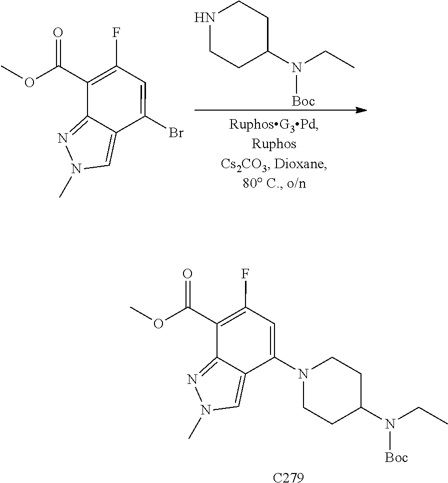 [SMILES: COC(=O)c1c(F)cc(Br)c2cn(C)nc12 ; CCN(C(=O)OC(C)(C)C)C1CCNCC1 ; CCN(C(=O)OC(C)(C)C)C1CCN(c2cc(F)c(C(=O)OC)c3nn(C)cc23)CC1 ; "C279"]

To a stirred mixture of methyl 4-bromo-6-fluoro-2-methylindazole-7-carboxylate (110 mg, 0.383 mmol, 1 equiv) and tert-butyl N-ethyl-N-(piperidin-4-yl)carbamate (87.49 mg, 0.383 mmol, 1 equiv), Cs₂CO₃ (374.52 mg, 1.149 mmol, 3 equiv) in 1,4-dioxane (2 mL) was added RuPhos (35.76 mg, 0.077 mmol, 0.2 equiv) and RuPhos Palladacycle Gen.3 (32.05 mg, 0.038 mmol, 0.10 equiv) at room temperature under nitrogen atmosphere. The resulting mixture was stirred overnight at 80° C. under nitrogen atmosphere. The resulting mixture was concentrated under vacuum. The residue was purified by silica gel column chromatography, eluted with PE/EA (1:2) to afford methyl 4-{4-[(tert-butoxycarbonyl)(ethyl)amino]piperidin-1-yl}-6-fluoro-2-methylindazole-7-carboxylate (C279, 145 mg, 87%) as a solid. LCMS (ES, m/z): 435 [M+H]*Synthesis of Intermediate 280

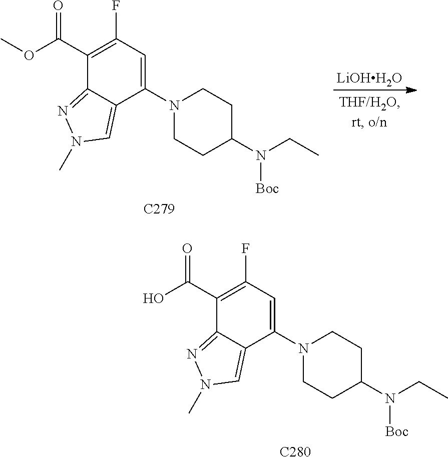 [SMILES: CCN(C(=O)OC(C)(C)C)C1CCN(c2cc(F)c(C(=O)OC)c3nn(C)cc23)CC1 ; "LiOH·H2O" ; "C279" ; CCN(C(=O)OC(C)(C)C)C1CCN(c2cc(F)c(C(=O)O)c3nn(C)cc23)CC1 ; "C280"]

To a stirred solution of methyl 4-{4-[(tert-butoxycarbonyl)(ethyl)amino]piperidin-1-yl}-6-fluoro-2-methylindazole-7-carboxylate (145 mg, 0.334 mmol, 1 equiv) in THF (1.2 mL) and H₂O (0.4 mL) was added lithiumol hydrate (42.01 mg, 1.002 mmol, 3 equiv) at room temperature. The resulting mixture was stirred overnight at room temperature. The resulting mixture was concentrated under vacuum. The resulting mixture was diluted with water. The mixture was acidified to pH 4 with citric acid. The resulting mixture was extracted with DCM (3×10 mL). The combined organic layers were washed with brine (1×10 mL), dried over anhydrous Na₂SO₄. After filtration, the filtrate was concentrated under reduced pressure to afford 4-{4-[(tert-butoxycarbonyl)(ethyl)amino]piperidin-1-yl}-6-fluoro-2-methylindazole-7-carboxylic acid (C280, 130 mg, 92%) as a solid. LCMS (ES, m/z): 421 [M−H]⁻

### Synthesis of Intermediate C281

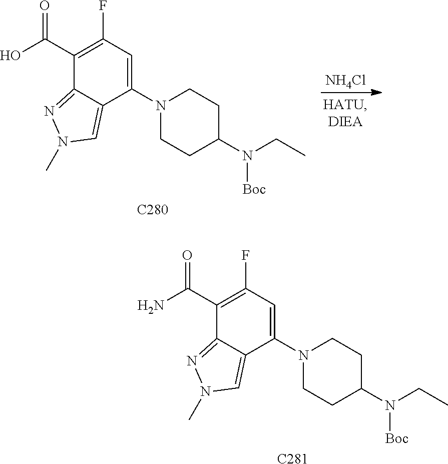 [SMILES: CCN(C(=O)OC(C)(C)C)C1CCN(c2cc(F)c(C(=O)O)c3nn(C)cc23)CC1 ; "NH4Cl" ; "C280" ; CCN(C(=O)OC(C)(C)C)C1CCN(c2cc(F)c(C(N)=O)c3nn(C)cc23)CC1 ; "C281"]

To a stirred mixture of 4-{4-[(tert-butoxycarbonyl)(ethyl)amino]piperidin-1-yl}-6-fluoro-2-methylindazole-7-carboxylic acid (130 mg, 0.309 mmol, 1 equiv) and NH₄Cl (66.15 mg, 1.236 mmol, 4 equiv) in DCM (2 mL) was added HATU (141.07 mg, 0.371 mmol, 1.2 equiv) and DIEA (199.79 mg, 1.545 mmol, 5 equiv) at room temperature. The resulting mixture was stirred overnight at room temperature. The resulting mixture was concentrated under vacuum. The residue was purified by silica gel column chromatography, eluted with PE/EA (1:4) to afford tert-butyl N-[1-(7-carbamoyl-6-fluoro-2-methylindazol-4-yl)piperidin-4-yl]-N-ethylcarbamate (C281, 110 mg, 84%) as solid. LCMS (ES, m/z): 420 [M−H]⁻

### Synthesis of Intermediate C282

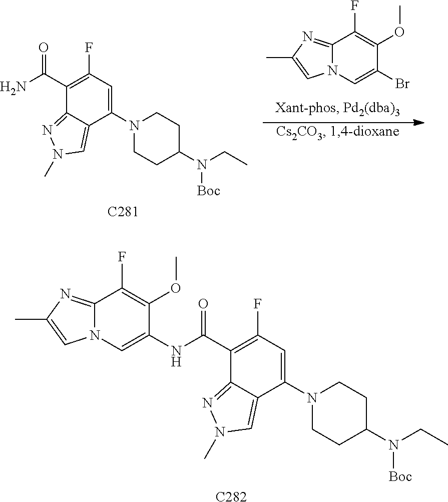

To a stirred mixture of tert-butyl N-[1-(7-carbamoyl-6-fluoro-2-methylindazol-4-yl)piperidin-4-yl]-N-ethylcarbamate (90 mg, 0.215 mmol, 1 equiv) and 6-bromo-8-fluoro-7-methoxy-2-methylimidazo[1,2-a]pyridine (66.70 mg, 0.258 mmol, 1.2 equiv) in 1,4-dioxane (2 mL) was added Cs₂CO₃ (209.70 mg, 0.645 mmol, 3 equiv), X-Phos (20.46 mg, 0.043 mmol, 0.2 equiv) and Pd₂(dba)₃ (19.65 mg, 0.022 mmol, 0.1 equiv) at room temperature under nitrogen atmosphere. The resulting mixture was stirred overnight at 80° C. under nitrogen atmosphere. The resulting mixture was concentrated under reduced pressure. The residue was purified by silica gel column chromatography, eluted with CH₂Cl₂/MeOH (10:1) to afford tert-butyl N-ethyl-N-{1-[6-fluoro-7-({8-fluoro-7-methoxy-2-methylimidazo[1,2-a]pyridin-6-yl}carbamoyl)-2-methylindazol-4-yl]piperidin-4-yl}carbamate (C282, 65 mg, 50%) as a solid. LCMS (ES, m/z): 596 [M−H]⁻

### Synthesis of Compound 460

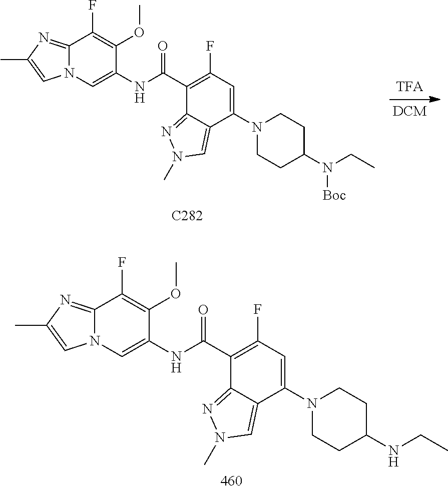

To a stirred solution of tert-butyl N-ethyl-N-{1-[6-fluoro-7-({8-fluoro-7-methoxy-2-methylimidazo[1,2-a]pyridin-6-yl}carbamoyl)-2-methylindazol-4-yl]piperidin-4-yl}carbamate (65 mg, 0.109 mmol, 1 equiv) in DCM (1 mL) was added TFA (1 mL) at room temperature. The resulting mixture was stirred for 2 hr at room temperature. The resulting mixture was concentrated under reduced pressure. The crude product was purified by Prep-HPLC (Condition 10, Gradient 10) to afford 4-[4-(ethylamino)piperidin-1-yl]-6-fluoro-N-{8-fluoro-7-methoxy-2-methylimidazo[1,2-a]pyridin-6-yl}-2-methylindazole-7-carboxamide (Compound 460, 15.3 mg, 28%) as a solid. LCMS (ES, m/z): 498 [M+H]^{+ 1}H NMR (400 MHz, DMSO-d₆) δ 11.56 (s, 1H), 9.46 (s, 1H), 8.83 (s, 1H), 7.76 (d, J=3.0 Hz, 1H), 6.23 (d, J=16.2 Hz, 1H), 4.27-4.17 (m, 6H), 3.93 (d, J=13.0 Hz, 2H), 3.14 (t, J=11.7 Hz, 2H), 2.71 (dd, J=8.8, 4.7 Hz, 1H), 2.60 (q, J=7.1 Hz, 2H), 2.31 (s, 3H), 1.99-1.90 (m, 2H), 1.40 (q, J=9.6 Hz, 2H), 1.04 (t, J=7.1 Hz, 3H).

### Example 191: Synthesis of Compound 462

### Synthesis of Intermediate C283

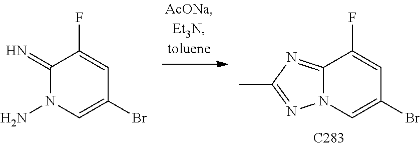

A solution of 5-bromo-3-fluoro-2-iminopyridin-1-amine (1.5 g, 7.281 mmol, 1 equiv), Et₃N (3.68 g, 36.405 mmol, 5 equiv) and Ac₂O (3.72 g, 36.405 mmol, 5 equiv) in toluene (30 mL) was stirred for 16 h at 100° C. The resulting mixture was concentrated under vacuum. The residue was purified by silica gel column chromatography, eluted with PE/EA (1:1) to afford 6-bromo-8-fluoro-2-methyl-[1,2,4]triazolo[1,5-a]pyridine (C283, 1 g, 59%) as a solid. LCMS (ES, m/z): 230 [M+H]⁺

### Synthesis of Intermediate C284

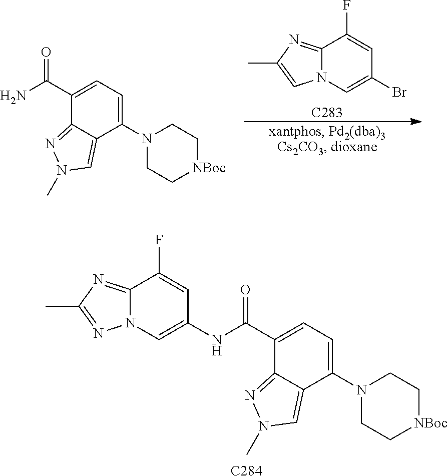

A mixture of tert-butyl 4-(7-carbamoyl-2-methylindazol-4-yl)piperazine-1-carboxylate (150 mg, 0.417 mmol, 1 equiv) and 6-bromo-8-fluoro-2-methyl-[1,2,4]triazolo[1,5-a]pyridine (115.20 mg, 0.500 mmol, 1.2 equiv), XantPhos (24.15 mg, 0.042 mmol, 0.1 equiv), Pd₂(dba)₃, (38.22 mg, 0.042 mmol, 0.1 equiv), Cs₂CO₃ (407.92 mg, 1.251 mmol, 3 equiv) in dioxane (2 mL) was stirred for 16 h at 100° C. under N₂ atmosphere. The resulting mixture was concentrated under reduced pressure. The residue was purified by silica gel column chromatography, eluted with DCM/MeOH to afford tert-butyl 4-[7-({8-fluoro-2-methyl-[1,2,4]triazolo[1,5-a]pyridin-6-yl}carbamoyl)-2-methylindazol-4-yl]piperazine-1-carboxylate (C284, 150 mg) as a solid. LCMS (ES, m/z): 509 [M+H]⁺

### Synthesis of Compound 462

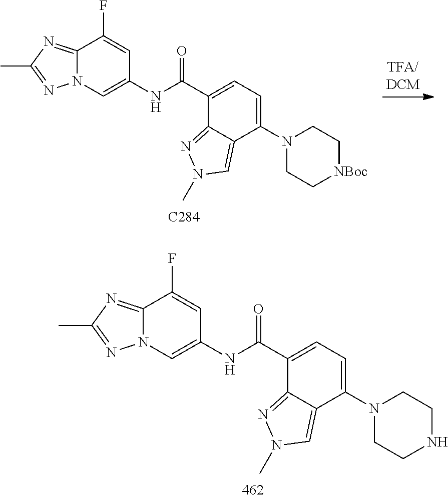

A mixture of tert-butyl 4-[7-({8-fluoro-2-methyl-[1,2,4]triazolo[1,5-a]pyridin-6-yl}carbamoyl)-2-methylindazol-4-yl]piperazine-1-carboxylate (150 mg, 0.295 mmol, 1 equiv) and TFA (0.4 mL, 5.385 mmol, 18.26 equiv) in DCM (2 mL) was stirred for 1 hr at 25° C. under N₂ atmosphere. The resulting mixture was concentrated under vacuum. The resulting mixture was concentrated under reduced pressure. The residue was purified by reverse flash chromatography (Condition 3, Gradient 12) to afford N-{8-fluoro-2-methyl-[1,2,4]triazolo[1,5-a]pyridin-6-yl}-2-methyl-4-(piperazin-1-yl) indazole-7-carboxamide (Compound 462, 63 mg, 52%) as a solid. LCMS (ES, m/z): 409 [M+H]^{+ 1}H NMR (300 MHz, DMSO-d6) δ 11.29 (s, 1H), 9.50 (d, J=1.6 Hz, 1H), 8.80 (s, 1H), 7.98 (d, J=8.1 Hz, 1H), 7.87 (dd, J=11.7, 1.7 Hz, 1H), 6.48 (d, J=8.2 Hz, 1H), 4.30 (s, 3H), 3.37 (t, J=4.9 Hz, 4H), 2.91 (t, J=4.8 Hz, 4H).

### Example 192: Synthesis of Compound 463

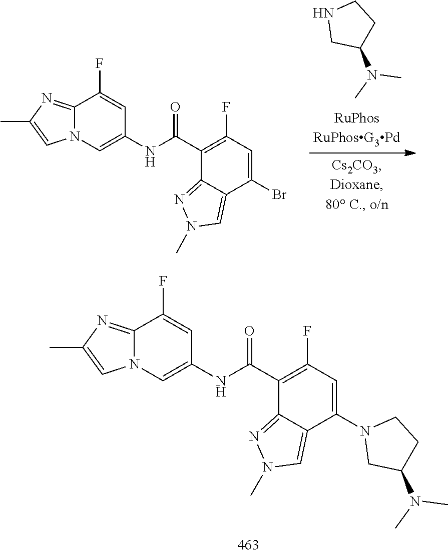

To a stirred mixture of 4-bromo-6-fluoro-N-{8-fluoro-2-methylimidazo[1,2-a]pyridin-6-yl}-2-methylindazole-7-carboxamide (80 mg, 0.190 mmol, 1 equiv) and (3R)—N,N-dimethylpyrrolidin-3-amine (21.74 mg, 0.190 mmol, 1 equiv), Cs₂CO₃ (186.09 mg, 0.570 mmol, 3 equiv) in 1,4-dioxane (1 mL) was added Ruphos (8.88 mg, 0.019 mmol, 0.1 equiv) and RuPhos Palladacycle Gen.3 (15.92 mg, 0.019 mmol, 0.1 equiv) at room temperature under nitrogen atmosphere. The resulting mixture was stirred overnight at 80° C. under nitrogen atmosphere. The resulting mixture was concentrated under vacuum. The residue was purified by silica gel column chromatography, eluted with CH₂Cl₂/MeOH (10:1) to afford 4-[(3R)-3-(dimethylamino)pyrrolidin-1-yl]-6-fluoro-N-{8-fluoro-2-methylimidazo[1,2-a]pyridin-6-yl}-2-methylindazole-7-carboxamide (55 mg, crude). The crude product was purified was purified by Prep-HPLC (Condition 10, Gradient 11) to afford 4-[(3R)-3-(dimethylamino)pyrrolidin-1-yl]-6-fluoro-N-{8-fluoro-2-methylimidazo[1,2-a]pyridin-6-yl}-2-methylindazole-7-carboxamide (Compound 463, 25.4 mg, 29%) as a solid. LCMS (ES, m/z): 454 [M−H]⁻ 1H NMR (400 MHz, DMSO-d6) δ 11.02 (s, 1H), 9.18 (d, J=1.6 Hz, 1H), 8.83 (s, 1H), 7.87 (d, J=3.1 Hz, 1H), 7.25 (dd, J=12.5, 1.7 Hz, 1H), 5.81 (d, J=16.0 Hz, 1H), 4.22 (s, 3H), 3.77 (dt, J=26.1, 9.0 Hz, 2H), 3.61 (d, J=8.0 Hz, 1H), 3.42 (d, J=9.0 Hz, 1H), 2.86 (p, J=7.6 Hz, 1H), 2.35 (s, 3H), 2.26 (s, 7H), 1.96-1.82 (m, 1H).

### Example 193: Synthesis of Compound 464

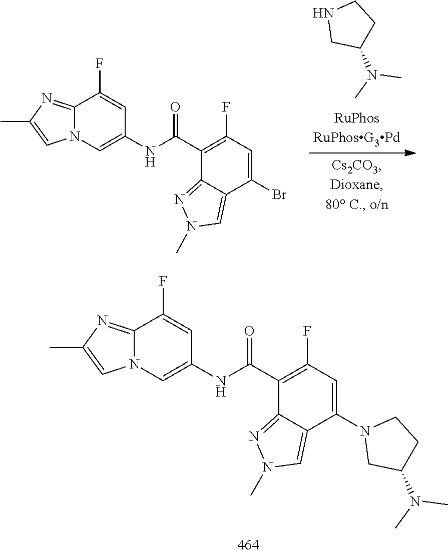

To a stirred mixture of 4-bromo-6-fluoro-N-{8-fluoro-2-methylimidazo[1,2-a]pyridin-6-yl}-2-methylindazole-7-carboxamide (80 mg, 0.190 mmol, 1 equiv) and (3S)—N,N-dimethylpyrrolidin-3-amine (21.74 mg, 0.190 mmol, 1 equiv), Cs₂CO₃ (186.09 mg, 0.570 mmol, 3 equiv) in 1,4-dioxane (1 mL) was added Ruphos (8.88 mg, 0.019 mmol, 0.1 equiv) and RuPhos Palladacycle Gen.3 (15.92 mg, 0.019 mmol, 0.1 equiv) at room temperature under nitrogen atmosphere. The resulting mixture was stirred overnight at 80° C. under nitrogen atmosphere. The resulting mixture was concentrated under vacuum. The residue was purified by silica gel column chromatography, eluted with CH₂Cl₂/MeOH (10:1) to afford 4-[(3S)-3-(dimethylamino)pyrrolidin-1-yl]-6-fluoro-N-{8-fluoro-2-methylimidazo[1,2-a]pyridin-6-yl}-2-methylindazole-7-carboxamide (50 mg, crude). The crude product was purified was purified by Prep-HPLC (Condition 10, Gradient 11) to afford 4-[(3S)-3-(dimethylamino)pyrrolidin-1-yl]-6-fluoro-N-{8-fluoro-2-methylimidazo[1,2-a]pyridin-6-yl}-2-methylindazole-7-carboxamide (Compound 464, 25.2 mg, 29%) as a solid.

LCMS (ES, m/z): 454 [M−H]⁻ 1H NMR (400 MHz, DMSO-d6) δ 11.02 (s, 1H), 9.18 (d, J=1.6 Hz, 1H), 8.83 (s, 1H), 7.87 (d, J=3.1 Hz, 1H), 7.25 (dd, J=12.5, 1.7 Hz, 1H), 5.81 (d, J=16.0 Hz, 1H), 4.22 (s, 3H), 3.77 (dt, J=26.1, 9.0 Hz, 2H), 3.61 (d, J=8.0 Hz, 1H), 3.42 (d, J=9.0 Hz, 1H), 2.86 (p, J=7.6 Hz, 1H), 2.35 (s, 3H), 2.26 (s, 7H), 1.96-1.82 (m, 1H).

### Example 194: Synthesis of Compound 470

### Synthesis of Intermediate C285

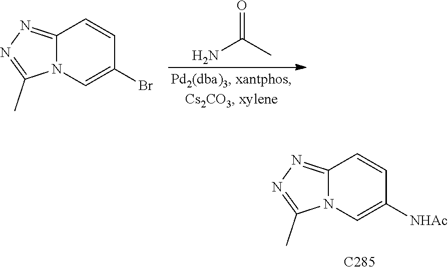

A solution of 6-bromo-3-methyl-[1,2,4]triazolo[4,3-a]pyridine (2 g, 9.432 mmol, 1 equiv) in dioxane (20 mL) was treated with acetamide (0.84 g, 14.148 mmol, 1.5 equiv), Pd₂(dba)₃ (0.86 g, 0.943 mmol, 0.1 equiv) xantphos (1.09 g, 1.886 mmol, 0.2 equiv), Cs₂CO₃ (6.15 g, 18.864 mmol, 2 equiv) for 8 hours at 80° C. under nitrogen atmosphere. The resulting mixture was filtered. the filter cake was washed with MeOH (3×50 mL). The filtrate was concentrated under reduced pressure. The residue was purified by silica gel column chromatography, eluted with PE/EA (10%-50%) to afford N-{3-methyl-[1,2,4]triazolo[4,3-a]pyridin-6-yl}acetamide (C285, 1.7 g, 94%) as a solid. LCMS (ES, m/z): 191 [M+H]⁺

### Synthesis of Intermediate C286

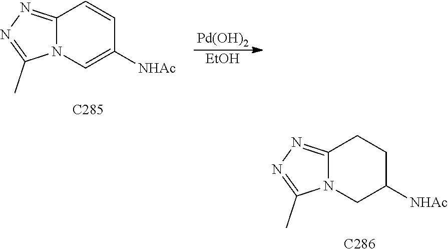

A solution N-{3-methyl-[1,2,4]triazolo[4,3-a]pyridin-6-yl}acetamide (1.5 g, 7.886 mmol, 1 equiv) and Pd(OH)₂/C (1.66 g, 11.829 mmol, 1.5 equiv) in EtOH (15 mL) was stirred for 120° C. at 16 hr under H₂ (30 atm) atmosphere. The resulting mixture was filtered and the filter cake was washed with ethanol (20 mL×3). The filtrate was concentrated under reduced pressure. The residue was purified by silica gel column chromatography, eluted with PE/EA to afford N-{3-methyl-5H,6H,7H,8H-[1,2,4]triazolo[4,3-a]pyridin-6-yl}acetamide (C286, 900 mg, 58%) as a solid. LCMS (ES, m/z): 195 [M+H]⁺

### Synthesis of Intermediate C287

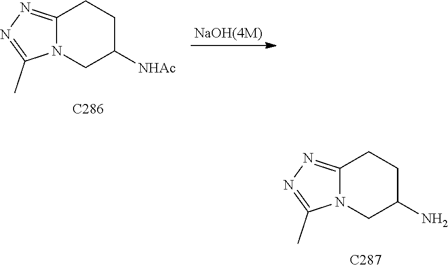

A mixture of N-{3-methyl-5H,6H,7H,8H-[1,2,4]triazolo[4,3-a]pyridin-6-yl}acetamide (300 mg, 1.544 mmol, 1 equiv) in NaOH (4 M, 3 mL) was stirred for 2 h at 100° C. under N₂ atmosphere. The resulting mixture was concentrated under reduced pressure to afford 3-methyl-5H,6H,7H,8H-[1,2,4]triazolo[4,3-a]pyridin-6-amine (C287, 300 mg crude) as a solid. LCMS (ES, m/z): 153 [M+H]⁺

### Synthesis of Intermediate C288

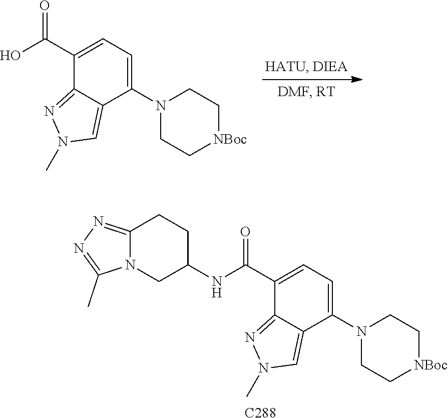

A solution 4-[4-(tert-butoxycarbonyl)piperazin-1-yl]-2-methylindazole-7-carboxylic acid (150 mg, 0.416 mmol, 1 equiv) and 3-methyl-5H,6H,7H,8H-[1,2,4]triazolo[4,3-a]pyridin-6-amine (C287, 190.03 mg, 1.248 mmol, 3 equiv), HATU (189.90 mg, 0.499 mmol, 1.2 equiv), DIEA (161.37 mg, 1.248 mmol, 3 equiv) in DMF (2 mL) was stirred for 2 hr at 25° C. under N₂ atmosphere. The residue was purified by silica gel column chromatography, eluted with DCM/MeOH to afford tert-butyl 4-[2-methyl-7-({3-methyl-5H,6H,7H,8H-[1,2,4]triazolo[4,3-a]pyridin-6-yl}carbamoyl) indazol-4-yl]piperazine-1-carboxylate (C288, 140 mg, 68%) as a solid. LCMS (ES, m/z): 495 [M+H]⁺

### Synthesis of Compound 470

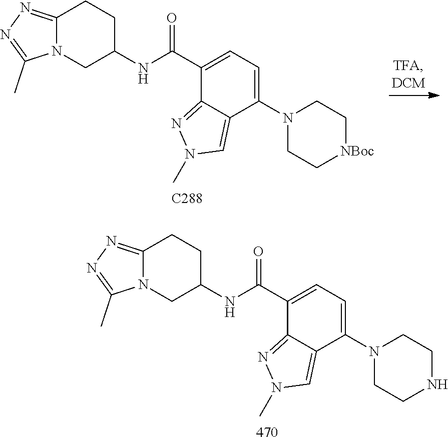

A mixture of tert-butyl 4-[2-methyl-7-({3-methyl-5H,6H,7H,8H-[1,2,4]triazolo[4,3-a]pyridin-6-yl}carbamoyl) indazol-4-yl]piperazine-1-carboxylate (120 mg, 0.243 mmol, 1 equiv) and TFA (0.2 mL, 2.693 mmol, 11.10 equiv) in DCM (1 mL) was stirred for 2 h at 25° C. The resulting mixture was concentrated under reduced pressure. The residue was purified by reverse flash chromatography (Condition 8, Gradient 1) to afford 2-methyl-N-{3-methyl-5H,6H,7H,8H-[1,2,4]triazolo[4,3-a]pyridin-6-yl}-4-(piperazin-1-yl) indazole-7-carboxamide (Compound 470, 23 mg, 24%) as a solid. LCMS (ES, m/z): 395 [M+H]^{+ 1}H NMR (300 MHz, DMSO-d6) δ 9.27 (d, J=7.2 Hz, 1H), 8.69 (s, 1H), 7.89 (d, J=8.0 Hz, 1H), 6.42 (d, J=8.1 Hz, 1H), 4.57 (d, J=6.2 Hz, 1H), 4.17 (dd, J=12.3, 4.6 Hz, 1H), 4.11 (s, 3H), 3.91 (dd, J=12.3, 5.8 Hz, 1H), 3.27 (s, 3H), 3.02 (t, J=6.9 Hz, 2H), 2.90 (s, 3H), 2.28 (s, 3H), 2.11 (d, J=7.2 Hz, 2H).

### Example 195: Synthesis of Compound 472

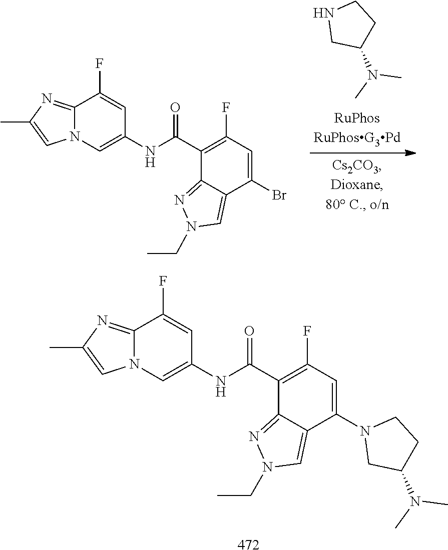

To a stirred mixture of 4-bromo-2-ethyl-6-fluoro-N-{8-fluoro-2-methylimidazo[1,2-a]pyridin-6-yl}indazole-7-carboxamide (250 mg, 0.576 mmol, 1 equiv), (3S)—N,N-dimethylpyrrolidin-3-amine (62.45 mg, 0.547 mmol, 0.95 equiv) and Cs₂CO₃ (562.73 mg, 1.728 mmol, 3 equiv) in 1,4-dioxane (5 mL) was added Ruphos (26.87 mg, 0.058 mmol, 0.1 equiv) and RuPhos Palladacycle Gen.3 (48.15 mg, 0.058 mmol, 0.1 equiv) at room temperature under nitrogen atmosphere. The resulting mixture was stirred overnight at 80° C. under nitrogen atmosphere. The resulting mixture was concentrated under vacuum. The residue was purified by silica gel column chromatography, eluted with CH₂Cl₂/MeOH (10:1) to afford 4-[(3S)-3-(dimethylamino)pyrrolidin-1-yl]-2-ethyl-6-fluoro-N-{8-fluoro-2-methylimidazo[1,2-a]pyridin-6-yl}indazole-7-carboxamide (35 mg, crude). The crude product was purified by Prep-HPLC (Condition 10, Gradient 5) to afford 4-[(3S)-3-(dimethylamino)pyrrolidin-1-yl]-2-ethyl-6-fluoro-N-{8-fluoro-2-methylimidazo[1,2-a]pyridin-6-yl}indazole-7-carboxamide (Compound 472, 7 mg, 2%) as solid. LCMS (ES, m/z): 468 [M+H]^{+ 1}H NMR (400 MHz, Methanol-d₄) δ 9.05 (d, J=1.6 Hz, 1H), 8.61 (s, 1H), 7.71 (d, J=2.9 Hz, 1H), 7.14 (dd, J=11.8, 1.6 Hz, 1H), 5.84 (d, J=16.2 Hz, 1H), 4.56 (q, J=7.3 Hz, 2H), 3.87 (dt, J=16.6, 9.0 Hz, 2H), 3.71 (q, J=10.2, 9.6 Hz, 1H), 3.49 (t, J=8.6 Hz, 1H), 3.03 (p, J=7.6 Hz, 1H), 2.43 (d, J=12.5 Hz, 9H), 2.38 (d, J=6.8 Hz, 1H), 2.02 (p, J=10.0 Hz, 1H), 1.69 (t, J=7.3 Hz, 3H),

### Example 196: Synthesis of Compound 473

### Synthesis of Intermediate C289

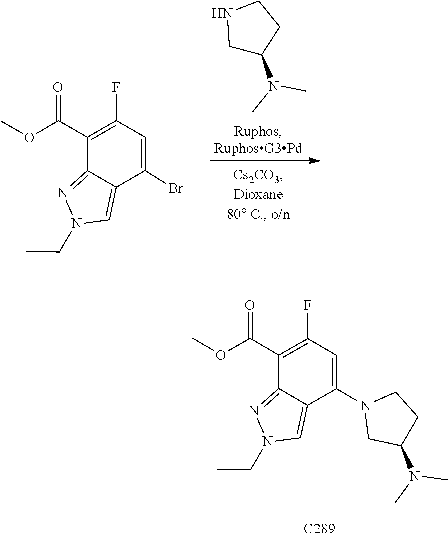

To a stirred mixture of methyl 4-bromo-2-ethyl-6-fluoroindazole-7-carboxylate (160 mg, 0.531 mmol, 1 equiv) and (3R)—N,N-dimethylpyrrolidin-3-amine (72.81 mg, 0.637 mmol, 1.2 equiv) in dioxane (3 mL) was added Cs₂CO₃ (519.38 mg, 1.593 mmol, 3 equiv), Ruphos (49.59 mg, 0.106 mmol, 0.2 equiv) and RuPhos Palladacycle Gen.3 (44.44 mg, 0.053 mmol, 0.1 equiv) at room temperature under nitrogen atmosphere. The resulting mixture was stirred overnight at 80° C. under nitrogen atmosphere. The resulting mixture was concentrated under reduced pressure. The residue was purified by silica gel column chromatography, eluted with CH₂Cl₂/MeOH (10:1) to afford methyl 4-[(3R)-3-(dimethylamino)pyrrolidin-1-yl]-2-ethyl-6-fluoroindazole-7-carboxylate (C289, 135 mg, 75%) as solid. LCMS (ES, m/z): 335 [M+H]⁺

### Synthesis of Intermediate C290

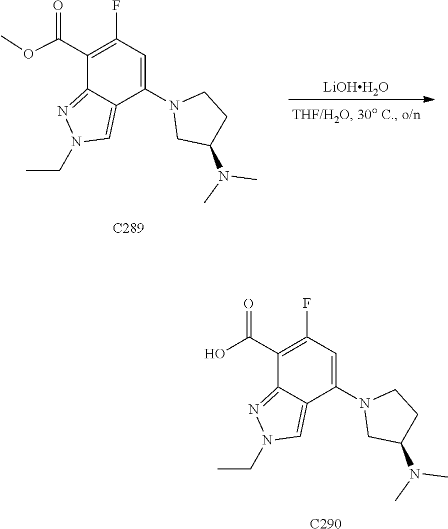

To a stirred solution of methyl 4-[(3R)-3-(dimethylamino)pyrrolidin-1-yl]-2-ethyl-6-fluoroindazole-7-carboxylate (135 mg, 0.404 mmol, 1 equiv) in THF (1.2 mL) and H₂O (0.4 mL) was added lithiumol hydrate (33.88 mg, 0.808 mmol, 2 equiv) at room temperature. The resulting mixture was stirred overnight at 30° C. The resulting mixture was concentrated under vacuum. The residue was dissolved in methanol (5 mL). The solution was added HCl(g) in MeOH (1 mL) dropwise at 0° C. The resulting mixture was stirred for 10 min at room temperature. The resulting mixture was concentrated under vacuum to afford 4-[(3R)-3-(dimethylamino)pyrrolidin-1-yl]-2-ethyl-6-fluoroindazole-7-carboxylic acid (C290, 150 mg) as a solid. LCMS (ES, m/z): 319 [M−H]⁻

### Synthesis of Compound 473

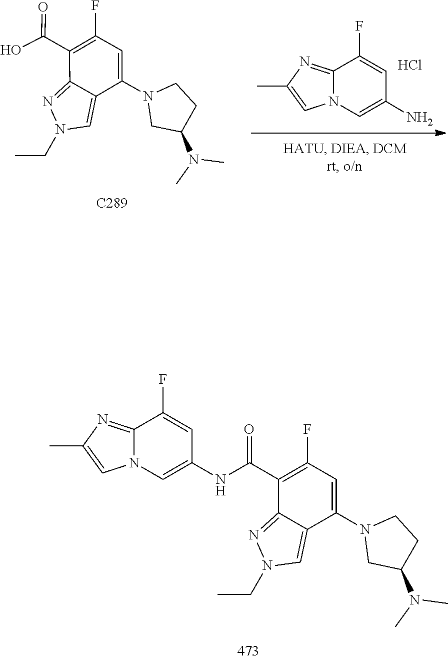

To a stirred mixture of 4-[(3R)-3-(dimethylamino)pyrrolidin-1-yl]-2-ethyl-6-fluoroindazole-7-carboxylic acid (150 mg, 0.468 mmol, 1 equiv) and 8-fluoro-2-methylimidazo[1,2-a]pyridin-6-amine hydrochloride (103.85 mg, 0.515 mmol, 1.1 equiv) in DCM (1.5 mL) was added HATU (231.44 mg, 0.608 mmol, 1.3 equiv) and DIEA (302.57 mg, 2.340 mmol, 5 equiv) at room temperature. The resulting mixture was stirred overnight at room temperature. The resulting mixture was concentrated under vacuum. The residue was purified by silica gel column chromatography, eluted with CH₂Cl₂/MeOH (10:1) to afford 4-[(3R)-3-(dimethylamino)pyrrolidin-1-yl]-2-ethyl-6-fluoro-N-{8-fluoro-2-methylimidazo[1,2-a]pyridin-6-yl}indazole-7-carboxamide (30 mg, crude). The crude product was purified by Prep-HPLC (Condition 15, Gradient 3) to afford 4-[(3R)-3-(dimethylamino)pyrrolidin-1-yl]-2-ethyl-6-fluoro-N-{8-fluoro-2-methylimidazo[1,2-a]pyridin-6-yl}indazole-7-carboxamide; bis(trifluoroacetic acid) (Compound 473, 16.9 mg, 5%) as a solid. LCMS (ES, m/z): 468 [M+H]^{+ 1}H NMR (400 MHz, Methanol-d₄) δ 9.41 (d, J=1.5 Hz, 1H), 8.72 (s, 1H), 8.05 (dd, J=2.4, 1.2 Hz, 1H), 7.84 (dd, J=11.4, 1.5 Hz, 1H), 5.98 (d, J=15.8 Hz, 1H), 4.60 (q, J=7.3 Hz, 2H), 4.18 (p, J=7.1 Hz, 2H), 4.03 (td, J=9.5, 3.2 Hz, 1H), 3.96-3.79 (m, 2H), 3.08 (s, 6H), 2.75-2.67 (m, 1H), 2.57 (d, J=0.9 Hz, H), 2.42 (dq, J=12.5, 8.5 Hz, 1H), 1.70 (t, J=7.3 Hz, 3H).

### Example 197: Synthesis of Compound 474

### Synthesis of Intermediate C290

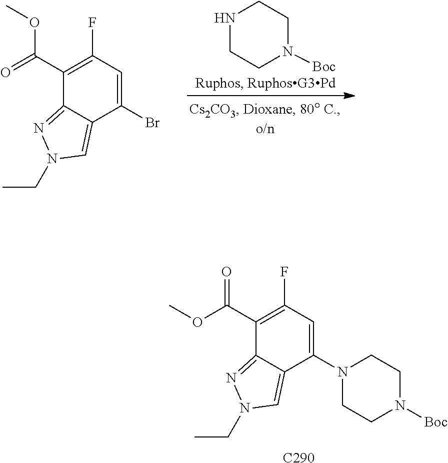 [SMILES: CCn1cc2c(Br)cc(F)c(C(=O)OC)c2n1 ; CC(C)(C)OC(=O)N1CCNCC1 ; CCn1cc2c(N3CCN(C(=O)OC(C)(C)C)CC3)cc(F)c(C(=O)OC)c2n1 ; "C290"]

To a stirred mixture of methyl 4-bromo-2-ethyl-6-fluoroindazole-7-carboxylate (110 mg, 0.365 mmol, 1 equiv) and tert-butyl piperazine-1-carboxylate (81.65 mg, 0.438 mmol, 1.2 equiv) in dioxane (2 mL) was added Cs₂CO₃ (357.07 mg, 1.095 mmol, 3 equiv), Ruphos (34.09 mg, 0.073 mmol, 0.2 equiv) and RuPhos Palladacycle Gen.3 (30.55 mg, 0.036 mmol, 0.1 equiv) at room temperature under nitrogen atmosphere. The resulting mixture was stirred overnight at 80° C. under nitrogen atmosphere. The resulting mixture was concentrated under reduced pressure. The residue was purified by silica gel column chromatography, eluted with PE/EA (1:1) to afford methyl 4-[4-(tert-butoxycarbonyl)piperazin-1-yl]-2-ethyl-6-fluoroindazole-7-carboxylate (90 mg, 60.61%) as a solid. LCMS (ES, m/z): 407 [M+H]⁺

### Synthesis of Intermediate C291

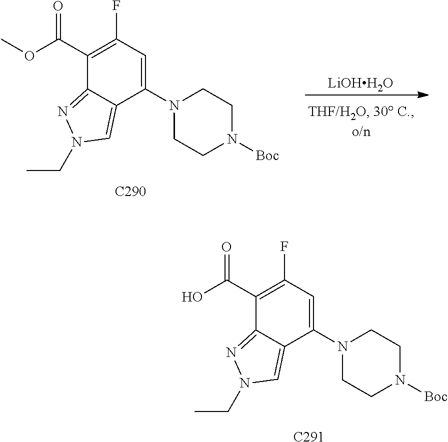 [SMILES: CCn1cc2c(N3CCN(C(=O)OC(C)(C)C)CC3)cc(F)c(C(=O)OC)c2n1 ; "LiOH·H2O" ; "C290" ; CCn1cc2c(N3CCN(C(=O)OC(C)(C)C)CC3)cc(F)c(C(=O)O)c2n1 ; "C291"]

To a stirred solution of methyl 4-[4-(tert-butoxycarbonyl)piperazin-1-yl]-2-ethyl-6-fluoroindazole-7-carboxylate (90 mg, 0.221 mmol, 1 equiv) in THF (1.2 mL) and H₂O (0.4 mL) was added lithiumol hydrate (18.58 mg, 0.442 mmol, 2 equiv) at room temperature. The resulting mixture was stirred overnight at 30° C. The resulting mixture was concentrated under vacuum. The resulting mixture was diluted with H₂O (5 mL). The mixture was acidified to pH 4 with citric acid. The resulting mixture was extracted with DCM (3×10 mL). The combined organic layers were washed with brine (1×10 mL), dried over anhydrous Na₂SO₄. After filtration, the filtrate was concentrated under reduced pressure to afford 4-[4-(tert-butoxycarbonyl)piperazin-1-yl]-2-ethyl-6-fluoroindazole-7-carboxylic acid (C291, 65 mg, 74%) as a solid. LCMS (ES, m/z): 391 [M−H]⁻

### Synthesis of Intermediate C292

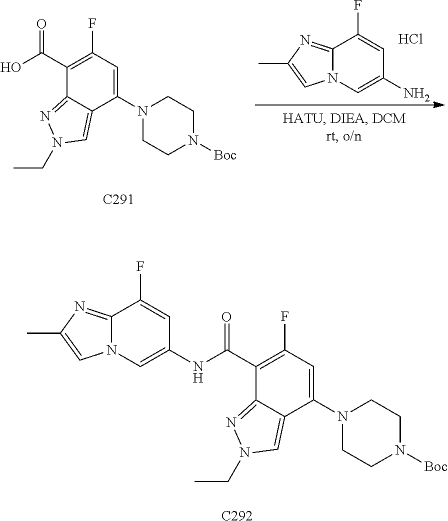

To a stirred mixture of 4-[4-(tert-butoxycarbonyl)piperazin-1-yl]-2-ethyl-6-fluoroindazole-7-carboxylic acid (65 mg, 0.166 mmol, 1 equiv) and 8-fluoro-2-methylimidazo[1,2-a]pyridin-6-amine hydrochloride (36.74 mg, 0.183 mmol, 1.1 equiv) in DCM (1 mL) was added DIEA (107.04 mg, 0.830 mmol, 5 equiv) and HATU (81.87 mg, 0.216 mmol, 1.3 equiv) at room temperature. The resulting mixture was stirred overnight at room temperature. The resulting mixture was concentrated under reduced pressure. The residue was purified by silica gel column chromatography, eluted with PE/EA (2:1) to afford tert-butyl 4-[2-ethyl-6-fluoro-7-({8-fluoro-2-methylimidazo[1,2-a]pyridin-6-yl}carbamoyl) indazol-4-yl]piperazine-1-carboxylate (C292, 70 mg, 78%) as a solid. LCMS (ES, m/z): 540 [M−H]⁻

### Synthesis of Compound 474

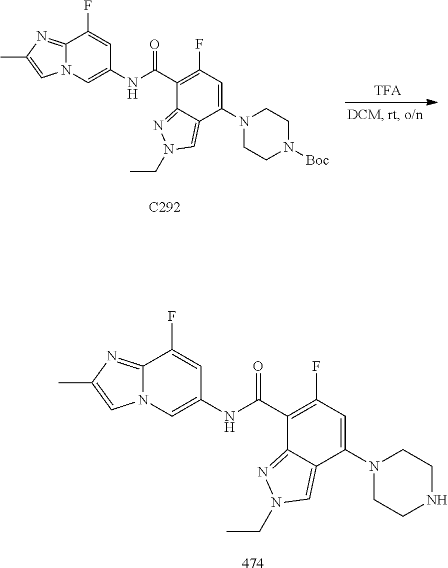

To a stirred solution of tert-butyl 4-[2-ethyl-6-fluoro-7-({8-fluoro-2-methylimidazo[1,2-a]pyridin-6-yl}carbamoyl) indazol-4-yl]piperazine-1-carboxylate (70 mg, 0.130 mmol, 1 equiv) in DCM (1 mL) was added TFA (1 mL) at room temperature. The resulting mixture was stirred for 2 h at room temperature. The resulting mixture was concentrated under reduced pressure. The crude product was purified by Prep-HPLC (Condition 10, Gradient 5) to afford 2-ethyl-6-fluoro-N-{8-fluoro-2-methylimidazo[1,2-a]pyridin-6-yl}-4-(piperazin-1-yl) indazole-7-carboxamide (Compound 473, 13.7 mg, 24%) as solid. LCMS (ES, m/z): 440 [M+H]^{+ 1}H NMR (400 MHz, DMSO-d₆) δ 10.94 (s, 1H), 9.20 (d, J=1.6 Hz, 1H), 8.79 (s, 1H), 7.90 (d, J=3.1 Hz, 1H), 7.21 (dd, J=12.4, 1.7 Hz, 1H), 6.23 (d, J=15.2 Hz, 1H), 4.50 (q, J=7.3 Hz, 2H), 3.36 (d, J=10.0 Hz, 4H), 2.90 (t, J=4.9 Hz, 4H), 2.35 (s, 3H), 1.56 (t, J=7.3 Hz, 3H).

### Example 198: Synthesis of Compound 476

### Synthesis of Intermediate C293

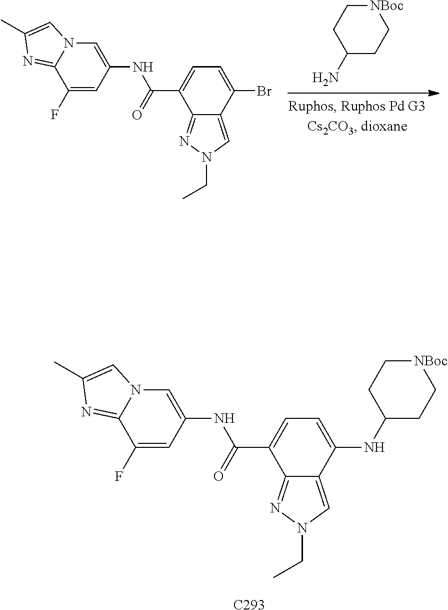 [SMILES: CCn1cc2c(Br)ccc(C(=O)Nc3cc(F)c4nc(C)cn4c3)c2n1 ; CC(C)(C)OC(=O)N1CCC(N)CC1 ; CCn1cc2c(NC3CCN(C(=O)OC(C)(C)C)CC3)ccc(C(=O)Nc3cc(F)c4nc(C)cn4c3)c2n1 ; "C293"]

To a solution of 4-bromo-2-ethyl-N-{8-fluoro-2-methylimidazo[1,2-a]pyridin-6-yl}indazole-7-carboxamide (200 mg, 0.480 mmol, 1 equiv) and tert-butyl 4-aminopiperidine-1-carboxylate (288.6 mg, 1.440 mmol, 3.0 equiv) in dioxane (10 mL) were added Cs₂CO₃ (469.6 mg, 1.440 mmol, 3.0 equiv), RuPhos (22.4 mg, 0.048 mmol, 0.1 equiv) and RuPhos Palladacycle Gen.3 (80.3 mg, 0.096 mmol, 0.2 equiv). After stirring for 3 h at 100° C. under a nitrogen atmosphere, the resulting mixture was concentrated under reduced pressure. The resulting mixture was concentrated under reduced pressure. The residue was purified by reverse flash chromatography (Condition 5, Gradient 5) to afford tert-butyl 4-{[2-ethyl-7-({8-fluoro-2-methylimidazo[1,2-a]pyridin-6-yl}carbamoyl) indazol-4-yl]amino}piperidine-1-carboxylate (C293, 120 mg, 46%) as a solid. LCMS (ES, m/z): 536 [M+H]⁺

### Synthesis of Compound 476

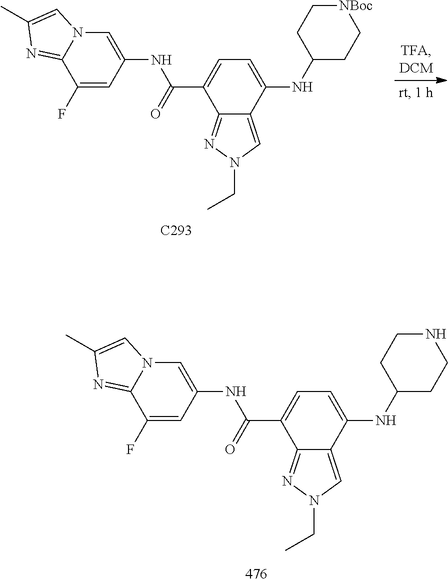

A solution of tert-butyl 4-{[2-ethyl-7-({8-fluoro-2-methylimidazo[1,2-a]pyridin-6-yl}carbamoyl) indazol-4-yl]amino}piperidine-1-carboxylate (70 mg, 0.131 mmol, 1 equiv) and TFA (0.2 mL) in DCM (2 mL) was stirred for 1 h at room temperature. The mixture was basified to pH 8 with NH₃(g) in MeOH. The resulting mixture was concentrated under reduced pressure. The residue was purified by reverse flash chromatography (Condition 3, Gradient 7) to afford 2-ethyl-N-{8-fluoro-2-methylimidazo[1,2-a]pyridin-6-yl}-4-(piperidin-4-ylamino) indazole-7-carboxamide (Compound 476, 27.4 mg, 48%) as a solid. LCMS (ES, m/z): 436 [M+H]^{+ 1}H NMR (300 MHz, DMSO-d₆) δ 10.90 (s, 1H), 9.18 (d, J=1.6 Hz, 1H), 8.74 (s, 1H), 7.96-7.86 (m, 2H), 7.27 (dd, J=12.4, 1.7 Hz, 1H), 6.89 (d, J=7.6 Hz, 1H), 6.19 (d, J=8.3 Hz, 1H), 4.56 (q, J=7.2 Hz, 2H), 3.55 (s, 1H), 3.00 (d, J=12.4 Hz, 2H), 2.60 (t, J=11.6 Hz, 2H), 2.35 (s, 3H), 1.95 (d, J=12.0 Hz, 2H), 1.61 (t, J=7.3 Hz, 3H), 1.39 (td, J=13.7, 12.2, 6.3 Hz, 2H).

### Example 199: Synthesis of Compound 477

### Synthesis of Intermediate C294

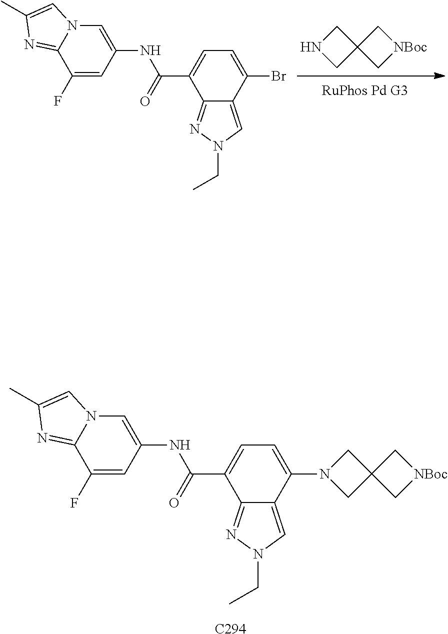

To a solution of 4-bromo-2-ethyl-N-{8-fluoro-2-methylimidazo[1,2-a]pyridin-6-yl}indazole-7-carboxamide (150 mg, 0.360 mmol, 1 equiv) and tert-butyl 2,6-diazaspiro[3.3]heptane-2-carboxylate (214.3 mg, 1.080 mmol, 3.0 equiv) in dioxane (4 mL) were added Cs₂CO₃ (82.3 mg, 1.080 mmol, 3.0 equiv), RuPhos (16.8 mg, 0.036 mmol, 0.1 equiv) and RuPhos Palladacycle Gen.3 (60.2 mg, 0.072 mmol, 0.2 equiv). After stirring for 12 hr at 100° C. under a nitrogen atmosphere, the resulting mixture was concentrated under reduced pressure. The residue was purified by reverse flash chromatography (Condition 5, Gradient 2) to afford tert-butyl 6-[2-ethyl-7-({8-fluoro-2-methylimidazo[1,2-a]pyridin-6-yl}carbamoyl) indazol-4-yl]-2,6-diazaspiro[3.3]heptane-2-carboxylate (C294, 160 mg, 83%) as a solid. LCMS (ES, m/z): 534 [M+H]⁺

### Synthesis of Compound 477

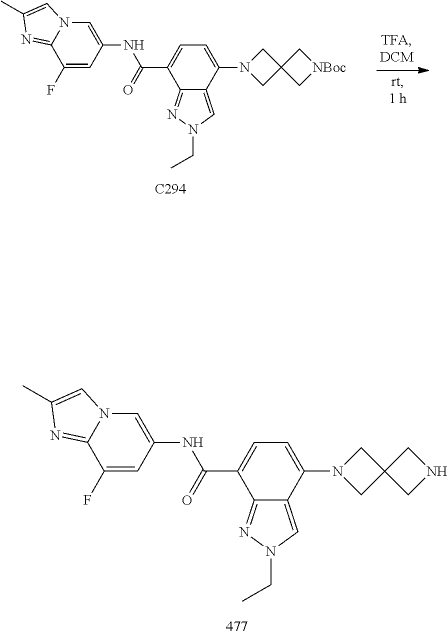

A solution of tert-butyl 6-[2-ethyl-7-({8-fluoro-2-methylimidazo[1,2-a]pyridin-6-yl}carbamoyl) indazol-4-yl]-2,6-diazaspiro[3.3]heptane-2-carboxylate (60 mg, 0.112 mmol, 1 equiv) and TFA (0.2 mL) in DCM (2 mL) was stirred for 1 h at room temperature. The mixture was basified to pH 8 with NH₃(g) in MeOH. The resulting mixture was concentrated under reduced pressure. The residue was purified by reverse flash chromatography with the following conditions: column, C18 silica gel; mobile phase, CH₃CN in water (0.05% NH₃·H₂O), 20% to 60% gradient in 10 min; detector, UV 254 nm. This resulted in 4-{2,6-diazaspiro[3.3]heptan-2-yl}-2-ethyl-N-{8-fluoro-2-methylimidazo[1,2-a]pyridin-6-yl}indazole-7-carboxamide (27.4 mg, 56.21%) as a light yellow solid.

LCMS (ES, m/z): 434 [M+H]^{+ 1}H NMR (300 MHz, DMSO-d₆) δ 10.96 (s, 1H), 9.18 (d, J=1.6 Hz, 1H), 8.67 (s, 1H), 7.97-7.86 (m, 2H), 7.27 (dd, J=12.4, 1.7 Hz, 1H), 5.90 (d, J=8.2 Hz, 1H), 4.58 (q, J=7.2 Hz, 2H), 4.35 (s, 4H), 4.07 (s, 1H), 3.77 (s, 3H), 2.38-2.32 (m, 3H), 1.61 (t, J=7.3 Hz, 3H).

### Example 200: Synthesis of Compound 478

### Synthesis of Intermediate C295

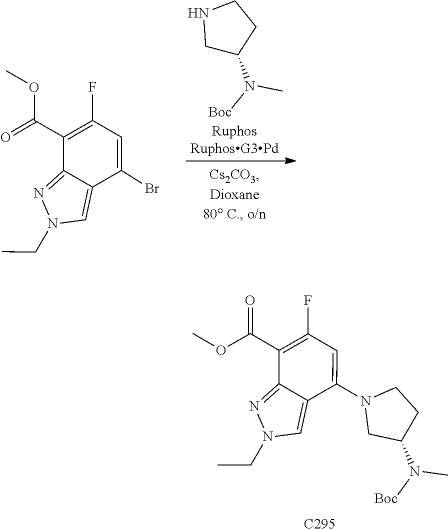

To a stirred mixture of methyl 4-bromo-2-ethyl-6-fluoroindazole-7-carboxylate (110 mg, 0.365 mmol, 1 equiv) and tert-butyl N-methyl-N-[(3S)-pyrrolidin-3-yl]carbamate (87.80 mg, 0.438 mmol, 1.2 equiv) in dioxane (2 mL) was added Cs₂CO₃ (357.07 mg, 1.095 mmol, 3 equiv), Ruphos (34.09 mg, 0.073 mmol, 0.2 equiv) and RuPhos Palladacycle Gen.3 (30.55 mg, 0.036 mmol, 0.1 equiv) at room temperature under nitrogen atmosphere. The resulting mixture was stirred overnight at 80° C. under nitrogen atmosphere. The resulting mixture was concentrated under reduced pressure. The residue was purified by silica gel column chromatography, eluted with PE/EA (1:1) to afford methyl 4-[(3S)-3-[(tert-butoxycarbonyl)(methyl)amino]pyrrolidin-1-yl]-2-ethyl-6-fluoroindazole-7-carboxylate (C295, 85 mg, 55%) as a solid. LCMS (ES, m/z): 421 [M+H]⁺

### Synthesis of Intermediate C296

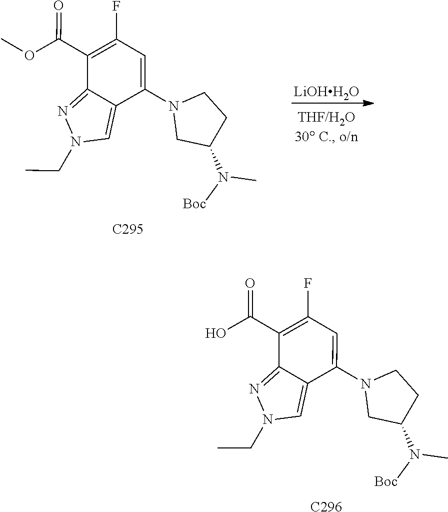

To a stirred solution of methyl 4-[(3S)-3-[(tert-butoxycarbonyl)(methyl)amino]pyrrolidin-1-yl]-2-ethyl-6-fluoroindazole-7-carboxylate (85 mg, 0.202 mmol, 1 equiv) in THF (1.2 mL) and H₂O (0.4 mL) was added lithium hydroxide hydrate (18.35 mg, 0.438 mmol, 2 equiv) at room temperature. The resulting mixture was stirred overnight at 30° C. The resulting mixture was concentrated under vacuum. The resulting mixture was diluted with H₂O (5 mL). The mixture was acidified to pH 4 with citric acid. The resulting mixture was extracted with DCM (3×10 mL). The combined organic layers were washed with brine (1×10 mL), dried over anhydrous Na₂SO₄. After filtration, the filtrate was concentrated under reduced pressure to afford 4-[(3S)-3-[(tert-butoxycarbonyl)(methyl)amino]pyrrolidin-1-yl]-2-ethyl-6-fluoroindazole-7-carboxylic acid (C296, 60 mg, 73%) as solid. LCMS (ES, m/z): 405 [M−H]⁻

### Synthesis of Intermediate C297

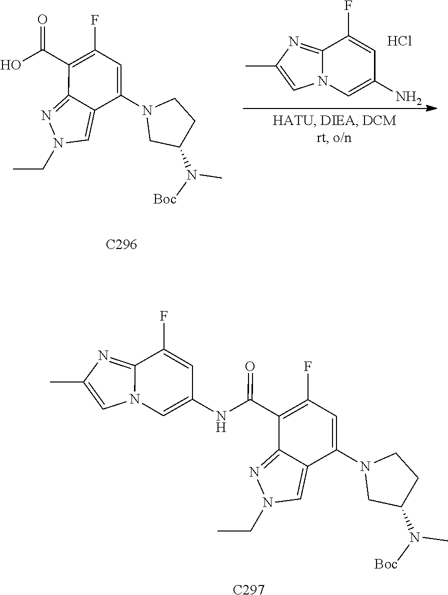

To a stirred mixture of 4-[(3S)-3-[(tert-butoxycarbonyl)(methyl)amino]pyrrolidin-1-yl]-2-ethyl-6-fluoroindazole-7-carboxylic acid (60 mg, 0.148 mmol, 1 equiv) and 8-fluoro-2-methylimidazo[1,2-a]pyridin-6-amine hydrochloride (32.74 mg, 0.163 mmol, 1.1 equiv) in DCM (2 mL) was added DIEA (95.40 mg, 0.740 mmol, 5 equiv) and HATU (72.97 mg, 0.192 mmol, 1.3 equiv) at room temperature. The resulting mixture was stirred overnight at room temperature. The resulting mixture was concentrated under reduced pressure. The residue was purified by silica gel column chromatography, eluted with PE/EA (2:1) to afford tert-butyl N-[(3S)-1-[2-ethyl-6-fluoro-7-({8-fluoro-2-methylimidazo[1,2-a]pyridin-6-yl}carbamoyl) indazol-4-yl]pyrrolidin-3-yl]-N-methylcarbamate (C297, 70 mg, 85%) as a solid. LCMS (ES, m/z): 554 [M+H]⁺

### Synthesis of Compound 478

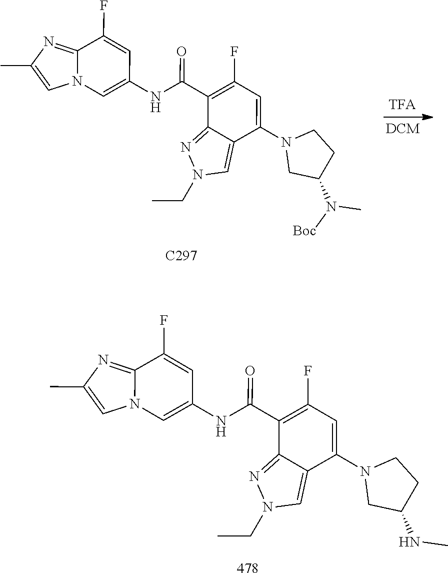

To a stirred solution of tert-butyl N-[(3S)-1-[2-ethyl-6-fluoro-7-({8-fluoro-2-methylimidazo[1,2-a]pyridin-6-yl}carbamoyl) indazol-4-yl]pyrrolidin-3-yl]-N-methylcarbamate (70 mg, 0.126 mmol, 1 equiv) in DCM (1 mL) was added TFA (1 mL) at room temperature. The resulting mixture was stirred for 1 hr at room temperature. The resulting mixture was concentrated under vacuum. The residue was purified by Prep-HPLC (Condition 15, Gradient 3) to afford 2-ethyl-6-fluoro-N-{8-fluoro-2-methylimidazo[1,2-a]pyridin-6-yl}-4-[(3S)-3-(methylamino)pyrrolidin-1-yl]indazole-7-carboxamide; bis(trifluoroacetic acid) (Compound 478, 14.6 mg, 16%) as solid. LCMS (ES, m/z): 454 [M+H]^{+ 1}H NMR (400 MHz, Methanol-d₄) δ 9.47 (d, J=1.6 Hz, 1H), 8.71 (s, 1H), 8.09 (dd, J=2.4, 1.2 Hz, 1H), 7.93 (dd, J=11.3, 1.5 Hz, 1H), 5.99 (d, J=15.8 Hz, 1H), 4.60 (q, J=7.3 Hz, 2H), 4.15-4.01 (m, 2H), 4.03-3.95 (m, 1H), 3.90 (h, J=6.7, 6.2 Hz, 2H), 2.88 (s, 3H), 2.65 (dt, J=13.5, 6.9 Hz, 1H), 2.59 (d, J=1.0 Hz, 3H), 2.41 (dt, J=11.9, 6.4 Hz, 1H), 1.70 (t, J=7.3 Hz, 3H).

### Example 201: Synthesis of Compound 479

### Synthesis of Intermediate C298

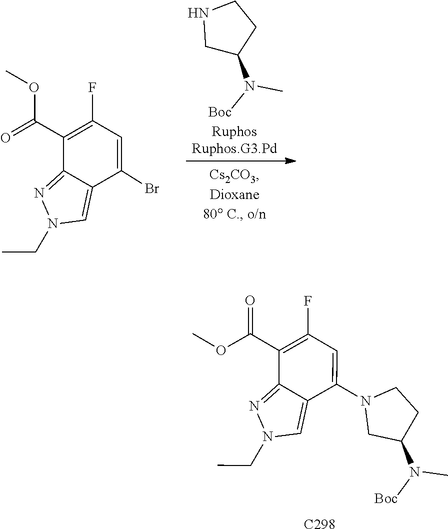

To a stirred mixture of methyl 4-bromo-2-ethyl-6-fluoroindazole-7-carboxylate (110 mg, 0.365 mmol, 1 equiv) and tert-butyl N-methyl-N-[(3R)-pyrrolidin-3-yl]carbamate (87.80 mg, 0.438 mmol, 1.2 equiv) in dioxane (2 mL) was added Cs₂CO₃ (357.07 mg, 1.095 mmol, 3 equiv), Ruphos (34.09 mg, 0.073 mmol, 0.2 equiv) and RuPhos Palladacycle Gen.3 (30.55 mg, 0.036 mmol, 0.1 equiv) at room temperature under nitrogen atmosphere. The resulting mixture was stirred overnight at 80° C. under nitrogen atmosphere. The resulting mixture was concentrated under reduced pressure. The residue was purified by silica gel column chromatography, eluted with PE/EA (1:1) to afford methyl 4-[(3R)-3-[(tert-butoxycarbonyl)(methyl)amino]pyrrolidin-1-yl]-2-ethyl-6-fluoroindazole-7-carboxylate (80 mg, 52%) as a solid. LCMS (ES, m/z): 421 [M+H]⁺

### Synthesis of Intermediate C299

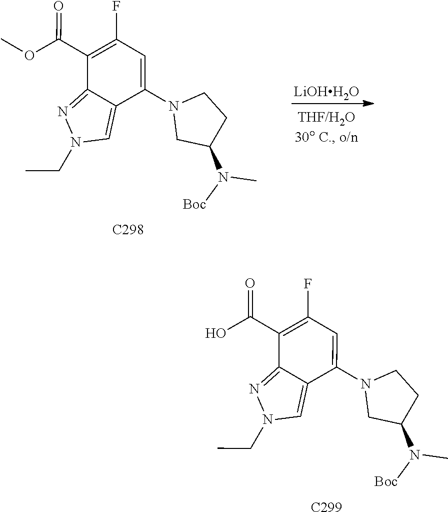

To a stirred solution of methyl 4-[(3R)-3-[(tert-butoxycarbonyl)(methyl)amino]pyrrolidin-1-yl]-2-ethyl-6-fluoroindazole-7-carboxylate (80 mg, 0.190 mmol, 1 equiv) in THF (1.2 mL) and H₂O (0.4 mL) was added lithiumol hydrate (15.97 mg, 0.380 mmol, 2 equiv) at room temperature. The resulting mixture was stirred overnight at 30° C. The resulting mixture was concentrated under vacuum. The resulting mixture was diluted with H₂O (5 mL). The mixture was acidified to pH 4 with citric acid. The resulting mixture was extracted with DCM (3×10 mL). The combined organic layers were washed with brine (1×10 mL), dried over anhydrous Na₂SO₄. After filtration, the filtrate was concentrated under reduced pressure to afford 4-[(3R)-3-[(tert-butoxycarbonyl)(methyl)amino]pyrrolidin-1-yl]-2-ethyl-6-fluoroindazole-7-carboxylic acid (C299, 55 mg, 71%) as a solid. LCMS (ES, m/z): 405 [M−H]⁻

### Synthesis of Intermediate C300

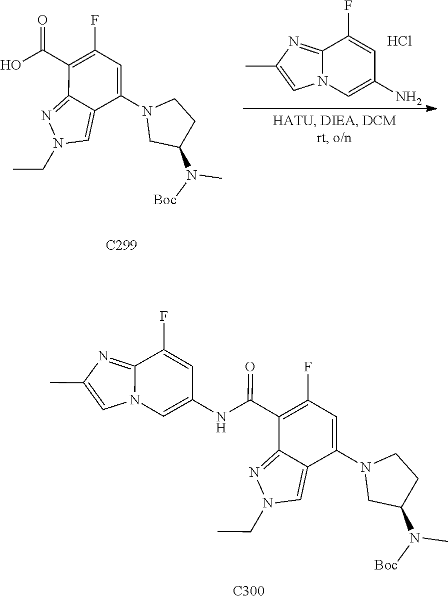

To a stirred mixture of 4-[(3R)-3-[(tert-butoxycarbonyl)(methyl)amino]pyrrolidin-1-yl]-2-ethyl-6-fluoroindazole-7-carboxylic acid (55 mg, 0.135 mmol, 1 equiv) and 8-fluoro-2-methylimidazo[1,2-a]pyridin-6-amine hydrochloride (30.01 mg, 0.149 mmol, 1.1 equiv) in DCM (1 mL) was added DIEA (87.45 mg, 0.675 mmol, 5 equiv) and HATU (66.89 mg, 0.176 mmol, 1.3 equiv) at room temperature. The resulting mixture was stirred overnight at room temperature. The resulting mixture was concentrated under reduced pressure. The residue was purified by silica gel column chromatography, eluted with PE/EA (2:1) to afford tert-butyl N-[(3R)-1-[2-ethyl-6-fluoro-7-({8-fluoro-2-methylimidazo[1,2-a]pyridin-6-yl}carbamoyl) indazol-4-yl]pyrrolidin-3-yl]-N-methylcarbamate (C300, 65 mg, 86%) as solid. LCMS (ES, m/z): 554 [M+H]⁺

### Synthesis of Compound 479

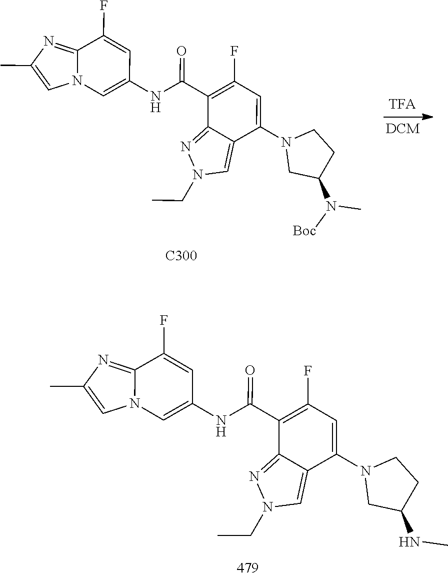

To a stirred solution of tert-butyl N-[(3R)-1-[2-ethyl-6-fluoro-7-({8-fluoro-2-methylimidazo[1,2-a]pyridin-6-yl}carbamoyl) indazol-4-yl]pyrrolidin-3-yl]-N-methylcarbamate (65 mg, 0.117 mmol, 1 equiv) in DCM (1 mL) was added TFA (1 mL) at room temperature. The resulting mixture was stirred for 1 h at room temperature. The resulting mixture was concentrated under vacuum. The residue was purified by Prep-HPLC (Condition 15, Gradient 3) to afford 2-ethyl-6-fluoro-N-{8-fluoro-2-methylimidazo[1,2-a]pyridin-6-yl}-4-[(3R)-3-(methylamino)pyrrolidin-1-yl]indazole-7-carboxamide; trifluoroacetic acid (Compound 479, 23.6 mg, 35%) as a solid. LCMS (ES, m/z): 454 [M+H]^{+ 1}H NMR (400 MHz, DMSO-d₆) δ 11.20 (s, 1H), 9.38 (s, 1H), 8.88 (s, 1H), 8.84 (s, 3H), 8.07 (s, 1H), 7.60 (s, 1H), 5.91 (d, J=15.6 Hz, 1H), 4.55 (q, J=7.3 Hz, 2H), 3.96 (dd, J=19.8, 8.5 Hz, 2H), 3.87 (d, J=8.8 Hz, 1H), 3.77 (d, J=10.0 Hz, 2H), 2.75-2.68 (m, 3H), 2.42 (s, 4H), 2.35-2.21 (m, 1H), 1.59 (t, J=7.2 Hz, 3H).

### Example 202: Synthesis of Compound 480

### Synthesis of Intermediate C301

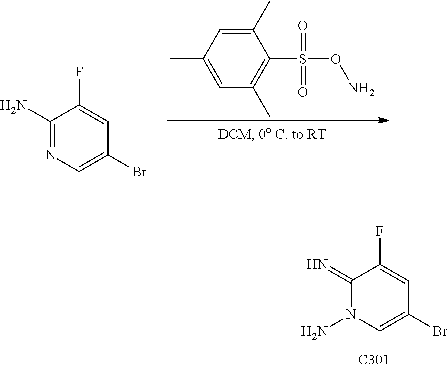

A solution of amino 2,4,6-trimethylbenzenesulfonate (5.41 g, 25.130 mmol, 1.2 equiv) in DCM (60 mL) was treated with 5-bromo-3-fluoropyridin-2-amine (4 g, 20.942 mmol, 1 equiv) for 10 mins at 0° C. The mixture was stirred for 8 hr at 20° C. The precipitated solids were collected by filtration and washed with DCM (20 mL). The residue was purified by trituration with ether (20 mL) to afford 5-bromo-3-fluoro-2-iminopyridin-1-amine (C301, 3.8 g, 88%) as a solid. LCMS (ES, m/z): 206 [M+H]⁺

### Synthesis of Intermediate C302

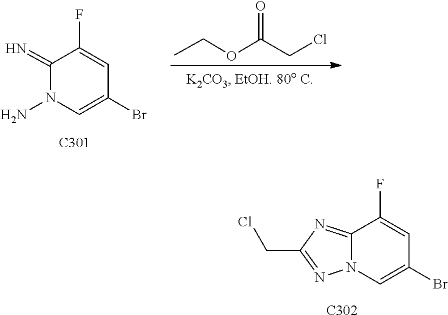

A solution of 5-bromo-3-fluoro-2-iminopyridin-1-amine (2 g, 9.708 mmol, 1 equiv) in EtOH (60 mL) was treated with ethyl chloroacetate (2.38 g, 19.416 mmol, 2 equiv), K₂CO₃ (2.68 g, 19.416 mmol, 2 equiv) for 4 hr at 80° C. The residue was purified by silica gel column chromatography, eluted with PE:EA (70%) to afford 6-bromo-2-(chloromethyl)-8-fluoro-[1,2,4]triazolo[1,5-a]pyridine (C302, 1 g, 38%) as a solid. LCMS (ES, m/z): 264 [M+H]⁺

### Synthesis of Intermediate C303

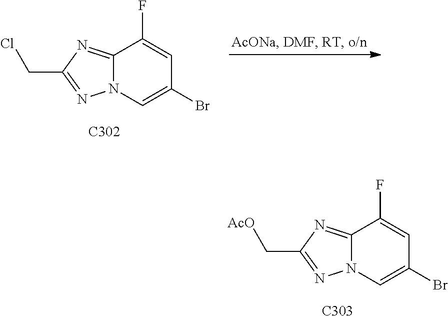

A solution of 6-bromo-2-(chloromethyl)-8-fluoro-[1,2,4]triazolo[1,5-a]pyridine (831 mg, 3.142 mmol, 1 equiv) in DMF (20 mL) was treated with AcONa (386 mg, 4.713 mmol, 1.5 equiv) for 8 hr at 20° C. The residue was purified by reverse flash chromatography (Condition 1, Gradient 1) to afford {6-bromo-8-fluoro-[1,2,4]triazolo[1,5-a]pyridin-2-yl}methyl acetate (C303, 500 mg, 55%) as a solid. LCMS (ES, m/z): 288 [M+H]⁺

### Synthesis of Intermediate C304

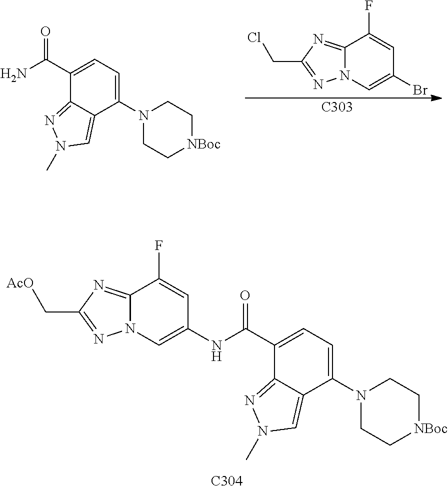

A solution of {6-bromo-8-fluoro-[1,2,4]triazolo[1,5-a]pyridin-2-yl}methyl acetate (270 mg, 0.937 mmol, 1.5 equiv) in dioxane (5 mL) was treated with tert-butyl 4-(7-carbamoyl-2-methylindazol-4-yl)piperazine-1-carboxylate (224 mg, 0.625 mmol, 1.00 equiv), XantPhos (72 mg, 0.125 mmol, 0.2 equiv), Pd₂(dba)₃ (57 mg, 0.062 mmol, 0.1 equiv), Cs₂CO₃ (407 mg, 1.249 mmol, 2 equiv) for 8 hours at 110° C. under nitrogen atmosphere. The resulting mixture was washed with water 100 mL. The residue was purified by silica gel column chromatography, eluted with DCM/MeOH 10:1. tert-butyl 4-[7-({2-[(acetyloxy)methyl]-8-fluoro-[1,2,4]triazolo[1,5-a]pyridin-6-yl}carbamoyl)-2-methylindazol-4-yl]piperazine-1-carboxylate (C304, 300 mg, 84%) as a solid.

LCMS (ES, m/z): 567 [M+H]⁺

### Synthesis of Intermediate C305

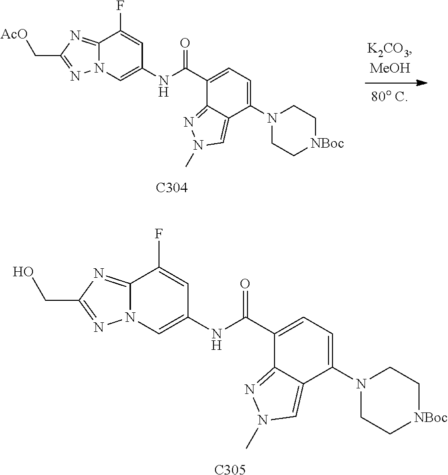

A solution of tert-butyl 4-[7-({2-[(acetyloxy)methyl]-8-fluoro-[1,2,4]triazolo[1,5-a]pyridin-6-yl}carbamoyl)-2-methylindazol-4-yl]piperazine-1-carboxylate (330 mg, 0.582 mmol, 1 equiv) in CH₃OH (10 mL) was treated with K₂CO₃ (241.48 mg, 1.746 mmol, 3 equiv) for 1 hour at 80° C. The resulting mixture was filtered. the filter cake was washed with CH₃OH (3×10 mL). The filtrate was concentrated under reduced pressure. The residue was purified by prep-HPLC (Condition 15, Gradient 4) to afford tert-butyl 4-(7-{[8-fluoro-2-(hydroxymethyl)-[1,2,4]triazolo[1,5-a]pyridin-6-yl]carbamoyl}-2-methylindazol-4-yl)piperazine-1-carboxylate (C305, 280 mg, 91%) as a solid.

LCMS (ES, m/z): 525[M+H]⁺

### Synthesis of Compound 480

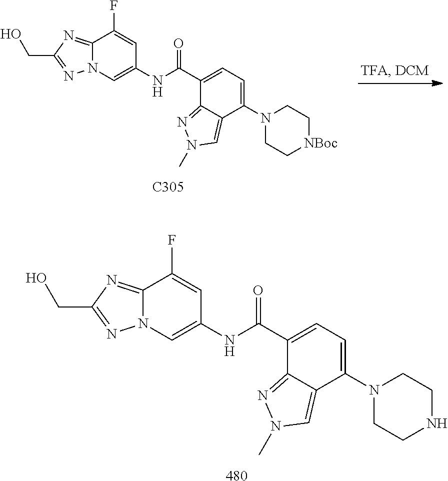

A solution of tert-butyl 4-(7-{[8-fluoro-2-(hydroxymethyl)-[1,2,4]triazolo[1,5-a]pyridin-6-yl]carbamoyl}-2-methylindazol-4-yl)piperazine-1-carboxylate (240 mg, 0.458 mmol, 1 equiv) in 1,4-dioxane (4 mL) was treated with HCl (4 mL) for 2 hr at 20° C. The resulting mixture was concentrated under reduced pressure. The residue was purified by reverse flash chromatography (Condition 1, Gradient 1) to afford N-[8-fluoro-2-(hydroxymethyl)-[1,2,4]triazolo[1,5-a]pyridin-6-yl]-2-methyl-4-(piperazin-1-yl) indazole-7-carboxamide (Compound 480, 2.2 mg, 1%) as a solid.

LCMS (ES, m/z): 425 [M+H]^{+ 1}H NMR (300 MHz, DMSO-d6) δ 11.33 (s, 1H), 9.57 (s, 1H), 8.82 (s, 1H), 7.97 (dd, J=17.9, 9.8 Hz, 2H), 6.50 (d, J=8.2 Hz, 1H), 5.55 (d, J=6.3 Hz, 1H), 4.67 (d, J=6.1 Hz, 2H), 4.31 (s, 3H), 2.91 (s, 4H).

### Example 203: Synthesis of Compound 481

### Synthesis of Intermediate C306

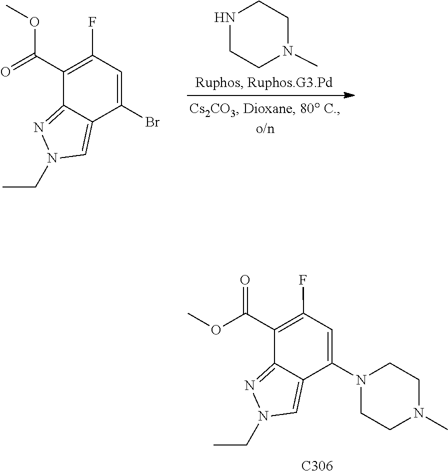

To a stirred mixture of methyl 4-bromo-2-ethyl-6-fluoroindazole-7-carboxylate (110 mg, 0.365 mmol, 1 equiv) and piperazine, 1-methyl- (43.91 mg, 0.438 mmol, 1.2 equiv) in dioxane (2 mL) was added Cs₂CO₃ (357.07 mg, 1.095 mmol, 3 equiv), Ruphos (34.09 mg, 0.073 mmol, 0.2 equiv) and RuPhos Palladacycle Gen.3 (30.55 mg, 0.036 mmol, 0.1 equiv) at room temperature under nitrogen atmosphere. The resulting mixture was stirred overnight at 80° C. under nitrogen atmosphere. The resulting mixture was concentrated under reduced pressure. The residue was purified by silica gel column chromatography, eluted with PE/EA (1:1) to afford methyl 2-ethyl-6-fluoro-4-(4-methylpiperazin-1-yl) indazole-7-carboxylate (C306, 90 mg, 76%) as a solid. LCMS (ES, m/z): 421 [M+H]⁺

### Synthesis of Intermediate C307

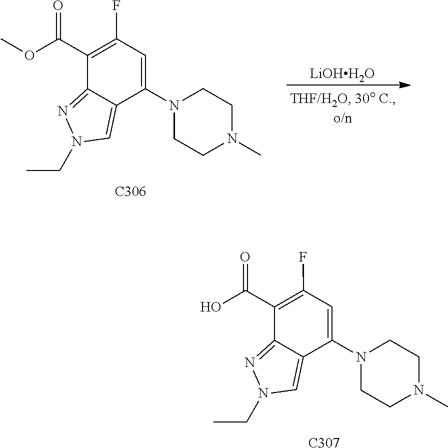

To a stirred solution of methyl 2-ethyl-6-fluoro-4-(4-methylpiperazin-1-yl) indazole-7-carboxylate (90 mg, 0.281 mmol, 1 equiv) in THF (1.2 mL) and H₂O (0.4 mL) was added lithium hydroxide hydrate (23.58 mg, 0.562 mmol, 2 equiv) at room temperature. The resulting mixture was stirred overnight at 30° C. The resulting mixture was concentrated under vacuum. The residue was dissolved in methanol (5 mL). The solution was added HCl(gas) in MeOH (1 mL) dropwise at 0° C. The resulting mixture was stirred for 10 min at room temperature. The resulting mixture was concentrated under vacuum to afford 2-ethyl-6-fluoro-4-(4-methylpiperazin-1-yl) indazole-7-carboxylic acid (C307, 145 mg, 84%) as a solid. LCMS (ES, m/z): 307 [M+H]⁺

### Synthesis of Compound 481

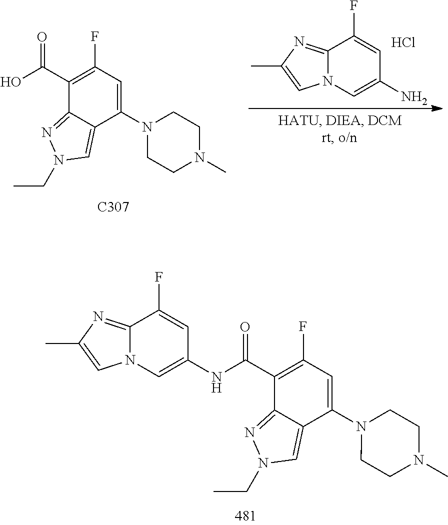

To a stirred mixture of 2-ethyl-6-fluoro-4-(4-methylpiperazin-1-yl) indazole-7-carboxylic acid (145 mg, 0.237 mmol, 1 equiv, 50%) and 8-fluoro-2-methylimidazo[1,2-a]pyridin-6-amine hydrochloride (52.49 mg, 0.261 mmol, 1.1 equiv) in DCM (3 mL) was added HATU (116.98 mg, 0.308 mmol, 1.3 equiv) and DIEA (152.94 mg, 1.185 mmol, 5 equiv) at room temperature. The resulting mixture was stirred overnight at room temperature. The resulting mixture was concentrated under reduced pressure. The residue was purified by silica gel column chromatography, eluted with CH₂Cl₂/MeOH (20:1) to afford crude product. The crude product was purified by Prep-HPLC (Condition 10, Gradient 12) to afford 2-ethyl-6-fluoro-N-{8-fluoro-2-methylimidazo[1,2-a]pyridin-6-yl}-4-(4-methylpiperazin-1-yl) indazole-7-carboxamide (Compound 481, 5.7 mg, 5%) as a solid. LCMS (ES, m/z): 454 [M+H]^{+ 1}H NMR (400 MHz, Methanol-d₄) δ 9.10 (d, J=1.6 Hz, 1H), 8.59 (s, 1H), 7.74 (d, J=2.9 Hz, 1H), 7.21 (dd, J=11.9, 1.7 Hz, 1H), 6.31 (d, J=15.5 Hz, 1H), 4.58 (q, J=7.3 Hz, 2H), 3.55 (t, J=5.0 Hz, 4H), 2.74-2.67 (m, 4H), 2.43 (d, J=12.4 Hz, 6H), 1.69 (t, J=7.3 Hz, 3H).

### Example 204: Synthesis of Compound 482

### Synthesis of Intermediate C308

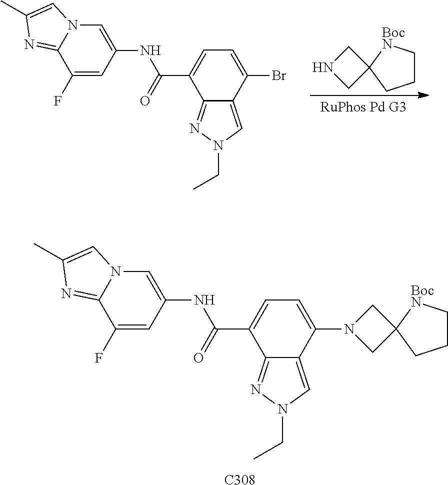

To a solution of 4-bromo-2-ethyl-N-{8-fluoro-2-methylimidazo[1,2-a]pyridin-6-yl}indazole-7-carboxamide (150 mg, 0.360 mmol, 1 equiv) and tert-butyl 2,5-diazaspiro[3.4]octane-5-carboxylate (153 mg, 0.720 mmol, 3.0 equiv) in dioxane (4 mL) were added Cs₂CO₃ (82.3 mg, 1.080 mmol, 3.0 equiv), RuPhos (16.8 mg, 0.036 mmol, 0.1 equiv) and RuPhos Palladacycle Gen.3 (60.2 mg, 0.072 mmol, 0.2 equiv). After stirring for 12 hr at 100° C. under a nitrogen atmosphere, the resulting mixture was concentrated under reduced pressure. The residue was purified by reverse flash chromatography (Condition 5, Gradient 2) to afford 2-[2-ethyl-7-({8-fluoro-2-methylimidazo[1,2-a]pyridin-6-yl}carbamoyl) indazol-4-yl]-2,5-diazaspiro[3.4]octane-5-carboxylate (C308, 120 mg, 91%) as a solid. LCMS (ES, m/z): 548 [M+H]⁺

### Synthesis of Compound 482

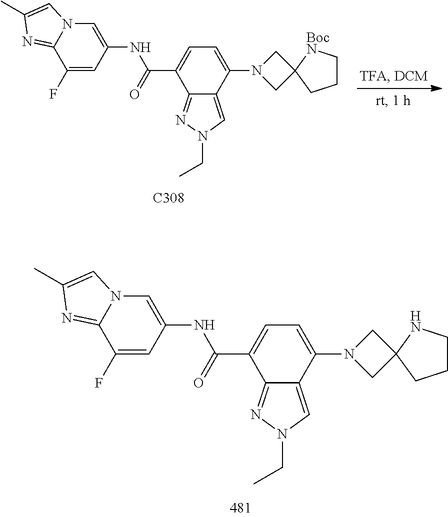

A solution of tert-butyl 2-[2-ethyl-7-({8-fluoro-2-methylimidazo[1,2-a]pyridin-6-yl}carbamoyl) indazol-4-yl]-2,5-diazaspiro[3.4]octane-5-carboxylate (100 mg, 0.183 mmol, 1 equiv) and TFA (0.2 mL) in DCM (2 mL) was stirred for 1 h at room temperature. The mixture was basified to pH 8 with NH₃(g) in MeOH. The resulting mixture was concentrated under reduced pressure. The residue was purified by reverse flash chromatography with the following conditions: column, C18 silica gel; mobile phase, CH₃CN in water (0.05% NH₃·H₂O), 20% to 60% gradient in 10 min; detector, UV 254 nm. This resulted in 4-{2,5-diazaspiro[3.4]octan-2-yl}-2-ethyl-N-{8-fluoro-2-methylimidazo[1,2-a]pyridin-6-yl}indazole-7-carboxamide (Compound 481, 25 mg, 30%) as a solid. LCMS (ES, m/z): 448 [M+H]^{+ 1}H NMR (300 MHz, DMSO-d₆) δ 10.97 (s, 1H), 9.19 (d, J=1.6 Hz, 1H), 8.67 (s, 1H), 7.98-7.86 (m, 2H), 7.27 (dd, J=12.4, 1.7 Hz, 1H), 5.90 (d, J=8.1 Hz, 1H), 4.57 (q, J=7.3 Hz, 2H), 4.25 (d, J=8.2 Hz, 2H), 4.13 (d, J=8.2 Hz, 2H), 2.91 (t, J=6.9 Hz, 2H), 2.35 (s, 3H), 2.02 (dd, J=8.3, 6.4 Hz, 2H), 1.77 (p, J=7.1 Hz, 2H), 1.61 (t, J=7.3 Hz, 3H).

### Example 205: Synthesis of Compound 390

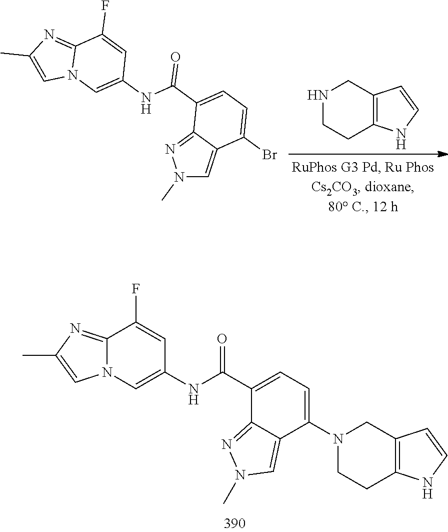 [SMILES: Cc1cn2cc(NC(=O)c3ccc(Br)c4cn(C)nc34)cc(F)c2n1 ; c1cc2c([nH]1)CCNC2 ; Cc1cn2cc(NC(=O)c3ccc(N4CCc5[nH]ccc5C4)c4cn(C)nc34)cc(F)c2n1]

To a stirred mixture of 4-bromo-N-{8-fluoro-2-methylimidazo[1,2-a]pyridin-6-yl}-2-methylindazole-7-carboxamide (50 mg, 0.124 mmol, 1 equiv) and 1H,4H,5H,6H,7H-pyrrolo[3,2-c]pyridine (18.22 mg, 0.149 mmol, 1.2 equiv) in dioxane (1 mL) was added Cs₂CO₃ (121.51 mg, 0.372 mmol, 3 equiv), RuPhos (11.60 mg, 0.025 mmol, 0.2 equiv) and RuPhos Palladacycle Gen.3 (10.40 mg, 0.012 mmol, 0.1 equiv) at room temperature under nitrogen atmosphere. The resulting mixture was stirred for 12 hr at 80° C. under nitrogen atmosphere. The resulting mixture was concentrated under reduced pressure. The residue was purified by silica gel column chromatography, eluted with CH₂Cl₂/MeOH (10:1) to afford crude product. The crude product was purified by Prep-HPLC (Condition 10, Gradient 2) to afford N-{8-fluoro-2-methylimidazo[1,2-a]pyridin-6-yl}-2-methyl-4-{1H,4H,6H,7H-pyrrolo[3,2-c]pyridin-5-yl}indazole-7-carboxamide (Compound 390, 16 mg, 29%) as solid. LCMS (ES, m/z): 444 [M+H]^{+ 1}H NMR (400 MHz, DMSO-d₆) δ 11.06 (s, 1H), 10.56 (s, 1H), 9.21 (s, 1H), 8.85 (s, 1H), 7.97 (d, J=8.1 Hz, 1H), 7.90 (d, J=3.1 Hz, 1H), 7.34 (d, J=12.7 Hz, 1H), 6.62 (t, J=2.6 Hz, 1H), 6.53 (d, J=8.4 Hz, 1H), 5.90 (t, J=2.5 Hz, 1H), 4.46 (s, 2H), 4.32 (s, 3H), 3.86 (t, J=5.5 Hz, 2H), 2.87 (t, J=5.4 Hz, 2H), 2.35 (s, 3H).

### Example 206: Synthesis of Compound 143

### Synthesis of Intermediate C309

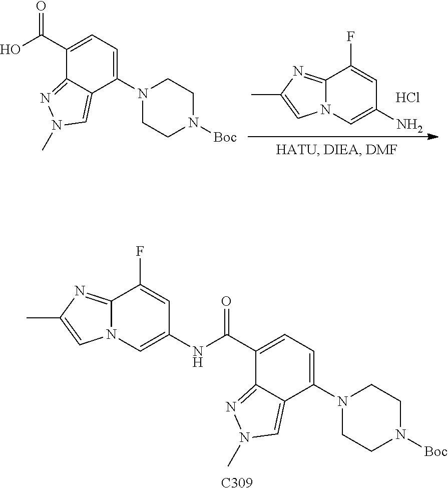

To a stirred mixture of 4-[4-(tert-butoxycarbonyl)piperazin-1-yl]-2-methylindazole-7-carboxylic acid (500 mg, 1.387 mmol, 1 equiv) and HATU (633.00 mg, 1.664 mmol, 1.2 equiv) in DMF (10 mL) was added DIEA (537.91 mg, 4.161 mmol, 3 equiv) and 8-fluoro-2-methylimidazo[1,2-a]pyridin-6-amine hydrochloride (335.66 mg, 1.664 mmol, 1.2 equiv) at room temperature. The resulting mixture was stirred overnight at room temperature. The resulting mixture was filtered. The filter cake was washed with methyl tert butyl ether (3×10 mL) and dried under infrared light to afford tert-butyl 4-[7-({8-fluoro-2-methylimidazo[1,2-a]pyridin-6-yl}carbamoyl)-2-methylindazol-4-yl]piperazine-1-carboxylate (C309, 460 mg, 65%) as solid. LCMS (ES, m/z): 508[M+H]⁺

### Synthesis of Compound 143

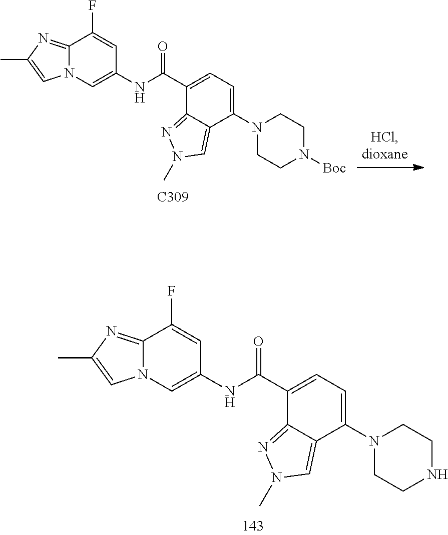

To a stirred solution of tert-butyl 4-[7-({8-fluoro-2-methylimidazo[1,2-a]pyridin-6-yl}carbamoyl)-2-methylindazol-4-yl]piperazine-1-carboxylate (460 mg, 0.906 mmol, 1 equiv) in dioxane (5 mL) was added HCl (gas) in 1,4-dioxane (5 mL) at room temperature. The resulting mixture was stirred for 2 h at room temperature. The resulting mixture was concentrated under reduced pressure. The resulting mixture was diluted with water (30 mL). The mixture was neutralized to pH 7 with saturated NaHCO₃ (aq.). The aqueous layer was extracted with dichloromethane (3×100 mL). The combined organic layers were concentrated under reduced pressure to afford N-{8-fluoro-2-methylimidazo[1,2-a]pyridin-6-yl}-2-methyl-4-(piperazin-1-yl) indazole-7-carboxamide (Compound 143, 297.7 mg, 80%) as solid. LCMS (ES, m/z): 408 [M+H]^{+ 1}H NMR (400 MHz, DMSO-d₆) δ 11.07 (s, 1H), 9.21 (d, J=1.6 Hz, 1H), 8.80 (s, 1H), 7.98 (d, J=8.0 Hz, 1H), 7.90 (d, J=3.1 Hz, 1H), 7.34 (dd, J=12.4, 1.7 Hz, 1H), 6.49 (d, J=8.2 Hz, 1H), 4.30 (s, 3H), 3.36 (t, J=5.0 Hz, 4H), 2.96-2.89 (m, 4H), 2.35 (s, 3H).

### Example 207: Synthesis of Compound 468

### Synthesis of Intermediate C310

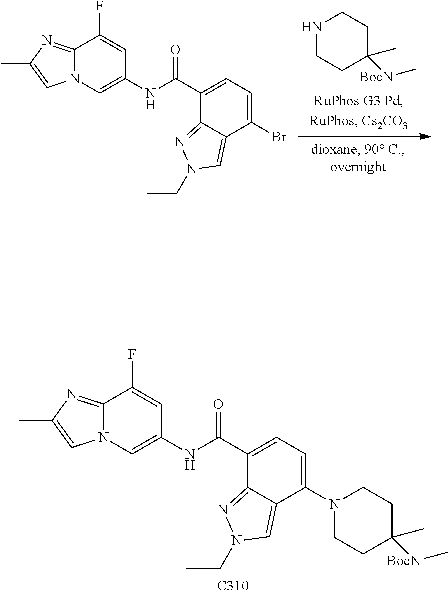

To a stirred solution of 4-bromo-2-ethyl-N-{8-fluoro-2-methylimidazo[1,2-a]pyridin-6-yl}indazole-7-carboxamide (180 mg, 0.432 mmol, 1 equiv) and tert-butyl N-methyl-N-(4-methylpiperidin-4-yl)carbamate (99 mg, 0.432 mmol, 1 equiv) in dioxane (5 mL) were added Cs₂CO₃ (423 mg, 1.296 mmol, 3 equiv), RuPhos (40 mg, 0.086 mmol, 0.2 equiv) and RuPhos Palladacycle Gen.3 (36 mg, 0.043 mmol, 0.1 equiv) in portions at room temperature under nitrogen atmosphere. The resulting mixture was stirred for overnight at 90° C. under nitrogen atmosphere. The mixture was allowed to cool down to room temperature. The resulting mixture was concentrated under reduced pressure. The residue was purified by silica gel column chromatography, eluted with PE/EA (1:10) to afford tert-butyl N-{1-[2-ethyl-7-({8-fluoro-2-methylimidazo[1,2-a]pyridin-6-yl}carbamoyl) indazol-4-yl]-4-methylpiperidin-4-yl}-N-methylcarbamate (C310, 210 mg, 86%) as a solid. LCMS (ES, m/z): 564 [M+H]⁺

### Synthesis of Compound 460

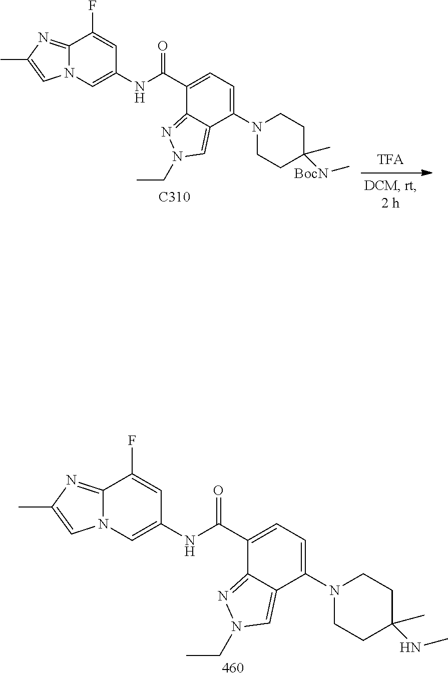

A solution of tert-butyl N-{1-[2-ethyl-7-({8-fluoro-2-methylimidazo[1,2-a]pyridin-6-yl}carbamoyl) indazol-4-yl]-4-methylpiperidin-4-yl}-N-methylcarbamate (200 mg, 0.355 mmol, 1 equiv) in TFA (2 mL) and DCM (2 mL) was stirred for 2 hr at room temperature. The resulting mixture was concentrated under reduced pressure. The residue was basified to pH 8 with 7 M NH₃(g) in MeOH. The resulting mixture was concentrated under vacuum. The residue was purified by reverse flash chromatography (Condition 3, Gradient 8) to afford 2-ethyl-N-{8-fluoro-2-methylimidazo[1,2-a]pyridin-6-yl}-4-[4-methyl-4-(methylamino)piperidin-1-yl]indazole-7-carboxamide (Compound 460, 50 mg, 30%) as a solid.

LCMS (ES, m/z): 464 [M+H]^{+ 1}H NMR (400 MHz, DMSO-d₆) δ 11.10 (s, 1H), 9.20 (d, J=1.6 Hz, 1H), 8.80 (s, 1H), 7.96 (d, J=8.1 Hz, 1H), 7.90 (dd, J=3.2, 1.0 Hz, 1H), 7.29 (dd, J=12.3, 1.7 Hz, 1H), 6.47 (d, J=8.2 Hz, 1H), 4.59 (q, J=7.3 Hz, 2H), 3.55-3.43 (m, 4H), 2.38-2.33 (m, 3H), 2.23 (s, 3H), 1.70 (dt, J=13.3, 4.6 Hz, 2H), 1.61 (q, J=7.4 Hz, 5H), 1.09 (s, 3H).

### Example 208: Synthesis of Compound 496

### Synthesis of Intermediate C311

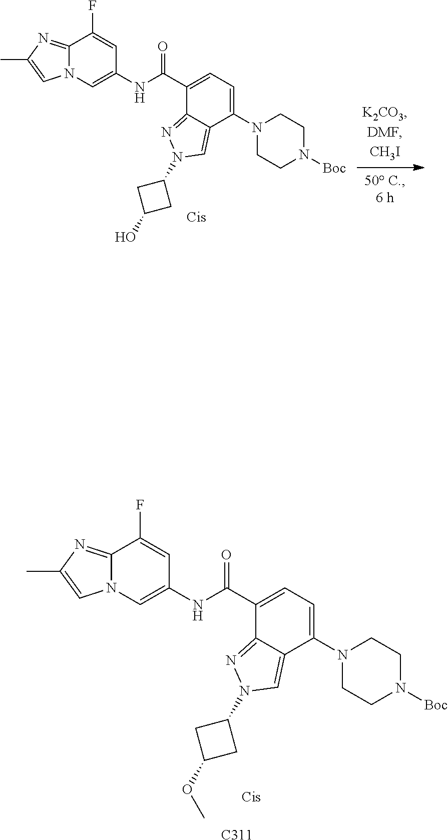

To a stirred mixture of Cis-tert-butyl 4-(7-((8-fluoro-2-methylimidazo[1,2-a]pyridin-6-yl)carbamoyl)-2-((1s,3s)-3-hydroxycyclobutyl)-2H-indazol-4-yl)piperazine-1-carboxylate (270.0 mg, 0.479 mmol, 1.0 equiv) and K₂CO₃ (133.3 mg, 0.958 mmol, 2.0 equiv), DMF (5 mL) were added methyl iodide (101.9 mg, 0.718 mmol, 1.5 equiv) dropwise at room temperature under nitrogen atmosphere. The resulting mixture was stirred for 6 hr at 50° C. under nitrogen atmosphere. The reaction was quenched by the addition of water (20 mL) at room temperature. The resulting mixture was extracted with ethyl acetate (2×20 mL). The combined organic layers were washed with brine (1×20 mL), dried over anhydrous Na₂SO₄. After filtration, the filtrate was concentrated under reduced pressure. The residue was purified by silica gel column chromatography, eluted with CH₂Cl₂/MeOH (20:1) to afford Cis-tert-butyl 4-(7-((8-fluoro-2-methylimidazo[1,2-a]pyridin-6-yl)carbamoyl)-2-((1s,3s)-3-methoxycyclobutyl)-2H-indazol-4-yl)piperazine-1-carboxylate (C311, 150 mg, 49%) as a solid. LCMS (ES, m/z): 578 [M+H]⁺

### Synthesis of Compound 496

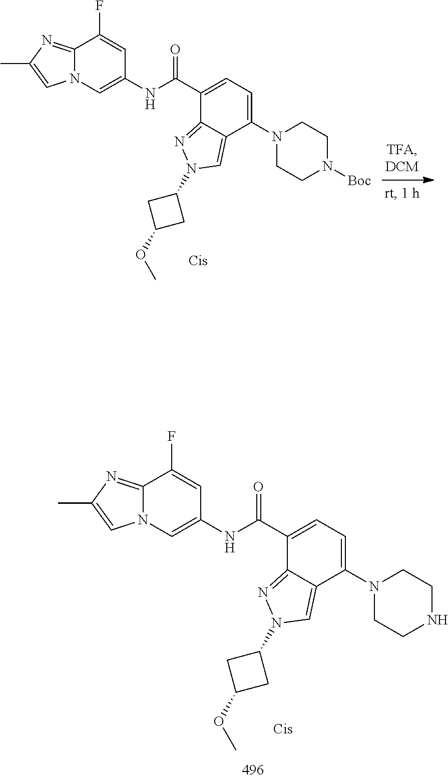 [SMILES: CO[C@H]1C[C@@H](n2cc3c(N4CCN(C(=O)OC(C)(C)C)CC4)ccc(C(=O)Nc4cc(F)c5nc(C)cn5c4)c3n2)C1 ; CO[C@H]1C[C@@H](n2cc3c(N4CCNCC4)ccc(C(=O)Nc4cc(F)c5nc(C)cn5c4)c3n2)C1]

To a stirred mixture of Cis-tert-butyl 4-(7-((8-fluoro-2-methylimidazo[1,2-a]pyridin-6-yl)carbamoyl)-2-((1s,3s)-3-methoxycyclobutyl)-2H-indazol-4-yl)piperazine-1-carboxylate (150.0 mg, 0.260 mmol, 1.0 equiv) in DCM (2 mL) were added TFA (1 mL) dropwise at room temperature. The resulting mixture was stirred for 1 hr at room temperature. The resulting mixture was concentrated under reduced pressure. The crude product was purified by Prep-HPLC (Condition 14, Gradient 2) to afford Cis-N-(8-fluoro-2-methylimidazo[1,2-a]pyridin-6-yl)-2-((1s,3s)-3-methoxycyclobutyl)-4-(piperazin-1-yl)-2H-indazole-7-carboxamide 2,2,2-trifluoroacetate (Compound 496, 70 mg, 44%) as a solid. LCMS (ES, m/z): 478 [M+H]^{+ 1}H NMR (400 MHz, DMSO-d₆) δ 11.52 (s, 1H), 9.68 (d, J=1.6 Hz, 1H), 9.11-9.07 (m, 2H), 9.03 (s, 1H), 8.41 (d, J=1.4 Hz, 1H), 8.12-8.02 (m, 2H), 6.64 (d, J=8.1 Hz, 1H), 5.62 (s, 1H), 4.96-4.84 (m, 1H), 4.17 (p, J=7.1 Hz, 1H), 4.01 (d, J=1.2 Hz, 3H), 3.63 (t, J=5.1 Hz, 4H), 3.37 (d, J=5.8 Hz, 4H), 2.98 (dhept, J=9.2, 2.5, 2.0 Hz, 2H), 2.66 (tdd, J=9.0, 7.5, 2.8 Hz, 2H), 2.55-2.50 (m, 3H).

### Example 209: Synthesis of Compound 420

### Synthesis of Intermediate C312

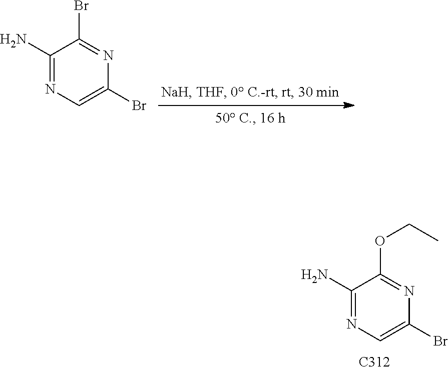

To a stirred solution of NaH (1.42 g, 59.313 mmol, 1.5 equiv) in THF (100 mL) was added ethanol (2.19 g, 47.450 mmol, 1.2 equiv) dropwise at 0° C. under nitrogen atmosphere. The resulting mixture was stirred for 0.5 h at room temperature under nitrogen atmosphere. To the above mixture was added 3,5-dibromopyrazin-2-amine (10.0 g, 39.542 mmol, 1.0 equiv) at room temperature. The resulting mixture was stirred for additional 16 hr at 50° C. The reaction was quenched with water at 0° C. The resulting mixture was extracted with ethyl acetate (2×100 mL). The combined organic layers were washed with water (1×200 mL), dried over anhydrous Na₂SO₄. After filtration, the filtrate was concentrated under reduced pressure. The residue was purified by silica gel column chromatography, eluted with PE/EA (2:1) to afford 5-bromo-3-ethoxypyrazin-2-amine (C312, 6 g, 69%) as a solid. LCMS (ES, m/z): 218 [M+H]⁺

### Synthesis of Intermediate C313

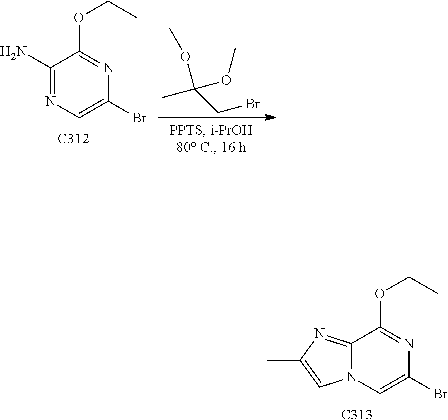

To a stirred mixture of 5-bromo-3-ethoxypyrazin-2-amine (3.40 g, 15.592 mmol, 1 equiv) and 1-bromo-2,2-dimethoxypropane (3.42 g, 18.710 mmol, 1.2 equiv) in i-PrOH (60 mL) was added PPTS (0.39 g, 1.559 mmol, 0.1 equiv) at room temperature. The resulting mixture was stirred for 16 hr at 80° C. under nitrogen atmosphere. The mixture was neutralized to pH 7 with saturated NaHCO₃ (aq.). The organic solvent was concentrated under vacuum. The residue was extracted with ethyl acetate (3×100 mL). The combined organic layers were washed with water (1×200 mL), dried over anhydrous Na₂SO₄. After filtration, the filtrate was concentrated under reduced pressure. The residue was purified by silica gel column chromatography, eluted with PE/EA (3:1) to afford 6-bromo-8-ethoxy-2-methylimidazo[1,2-a]pyrazine (C313, 1.9 g, 47%) as a solid. LCMS (ES, m/z): 256 [M+H]⁺

### Synthesis of Intermediate C314

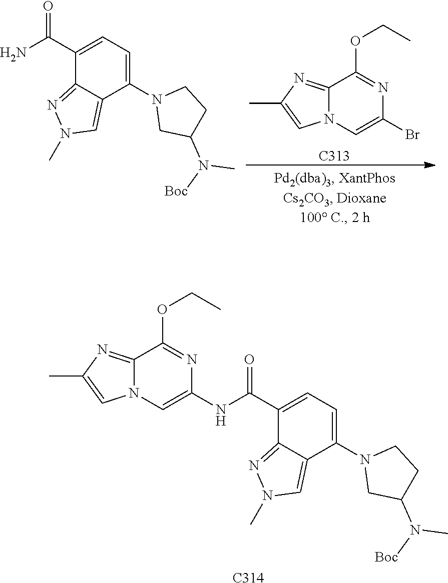

To a stirred mixture of tert-butyl N-[1-(7-carbamoyl-2-methylindazol-4-yl)pyrrolidin-3-yl]-N-methylcarbamate (280.0 mg, 0.577 mmol, 1 equiv, 77%) and 6-bromo-8-ethoxy-2-methylimidazo[1,2-a]pyrazine (221.8 mg, 0.865 mmol, 1.5 equiv) in dioxane (6 mL) were added Cs₂CO₃ (564.3 mg, 1.731 mmol, 3 equiv) and XantPhos (66.8 mg, 0.115 mmol, 0.2 equiv) and Pd₂(dba)₃ (52.9 mg, 0.058 mmol, 0.1 equiv) at room temperature under nitrogen atmosphere. The resulting mixture was stirred for 2 hr at 100° C. under nitrogen atmosphere. The mixture was allowed to cool down to room temperature. The resulting mixture was diluted with water (5 mL). The resulting mixture was extracted with ethyl acetate (3×5 mL). The combined organic layers were washed with brine (1×5 mL), dried over anhydrous Na₂SO₄. After filtration, the filtrate was concentrated under reduced pressure. The residue was purified by silica gel column chromatography, eluted with EA to afford tert-butyl N-{1-[7-({8-ethoxy-2-methylimidazo[1,2-a]pyrazin-6-yl}carbamoyl)-2-methylindazol-4-yl]pyrrolidin-3-yl}-N-methylcarbamate (C314, 180 mg, 56%) as a solid. LCMS (ES, m/z): 549 [M+H]⁺

### Synthesis of Compound 420

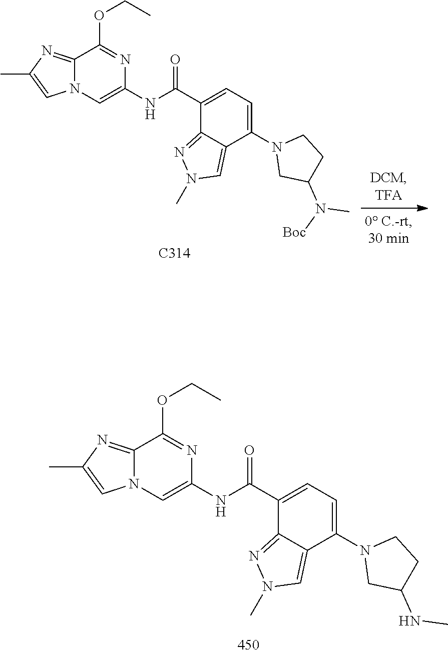

To a stirred mixture of tert-butyl N-{1-[7-({8-ethoxy-2-methylimidazo[1,2-a]pyrazin-6-yl}carbamoyl)-2-methylindazol-4-yl]pyrrolidin-3-yl}-N-methylcarbamate (170 mg, 0.310 mmol, 1 equiv) in DCM (3 mL) was added TFA (0.6 mL) dropwise at 0° C. The resulting mixture was stirred for 30 min at room temperature. The resulting mixture was concentrated under reduced pressure. The crude product was purified by Prep-HPLC (Condition 10, Gradient 2) to afford N-{8-ethoxy-2-methylimidazo[1,2-a]pyrazin-6-yl}-2-methyl-4-[3-(methylamino)pyrrolidin-1-yl]indazole-7-carboxamide (80 mg, 57%) as a solid. LCMS (ES, m/z): 449 [M+H]^{+ 1}H NMR (400 MHz, DMSO-d₆) δ 11.18 (s, 1H), 8.97 (s, 1H), 8.83 (s, 1H), 7.94 (d, J=8.3 Hz, 1H), 7.89 (d, J=1.0 Hz, 1H), 6.02 (d, J=8.4 Hz, 1H), 4.57 (q, J=7.1 Hz, 2H), 4.24 (s, 3H), 3.83-3.73 (m, 2H), 3.65 (d, J=7.5 Hz, 1H), 3.44-3.31 (m, 2H), 2.50 (s, 3H), 2.49 (s, 3H), 2.16 (dt, J=13.0, 6.3 Hz, 1H), 1.94 (dt, J=11.8, 6.0 Hz, 1H), 1.47 (t, J=7.1 Hz, 3H).

### Example 210: Synthesis of Compound 483

### Synthesis of Intermediate C315

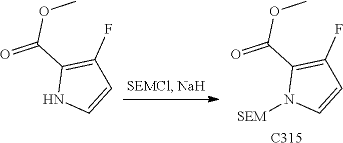

A solution of methyl 3-fluoro-1H-pyrrole-2-carboxylate (10 g, 69.873 mmol, 1 equiv) in THF (100 mL) was treated with NaH (2.52 g, 104.810 mmol, 1.5 equiv) for 0.5 h at 0° C. under nitrogen atmosphere followed by the addition of SESCl (16.83 g, 83.848 mmol, 1.2 equiv) dropwise at 25° C. was stirred for 2 hr. The resulting mixture was extracted with EA (50 mL×2). The combined organic layers were washed with brine (20 mL×2), dried over anhydrous Na₂SO₄. After filtration, the filtrate was concentrated under reduced pressure. The residue was purified by silica gel column chromatography, eluted with PE:EA (0%˜ 50%) to afford methyl 3-fluoro-1-{[2-(trimethylsilyl)ethoxy]methyl}pyrrole-2-carboxylate (C315, 18 g, 94%) as a solid. ¹H NMR (300 MHz, DMSO-d6) δ 11.68 (s, 1H), 6.87 (ddd, J=4.7, 3.8, 2.9 Hz, 1H), 6.03 (t, J=2.8 Hz, 1H), 4.24 (q, J=7.1 Hz, 2H), 1.27 (t, J=7.1 Hz, 3H).

### Synthesis of Intermediate C316

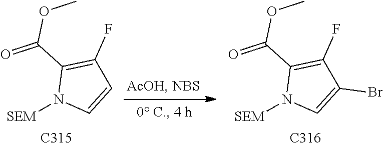

A solution of methyl 3-fluoro-1-{[2-(trimethylsilyl)ethoxy]methyl}pyrrole-2-carboxylate (19 g, 69.501 mmol, 1 equiv) and N-bromosuccinimide (13.61 g, 76.451 mmol, 1.1 equiv) in HOAc (150 mL, 2617.731 mmol, 37.66 equiv) was stirred for 2 hr at 25° C. under N₂ atmosphere. The mixture was basified to pH 8 with Na₂CO₃. The residue was purified by silica gel column chromatography, eluted with PE:EA (0%˜40%) to afford methyl 4-bromo-3-fluoro-1-{[2-(trimethylsilyl)ethoxy]methyl}pyrrole-2-carboxylate (C316, 3 g, 12%) as a solid. ¹H NMR (300 MHz, DMSO-d6) δ 7.54 (d, J=4.8 Hz, 1H), 5.54 (d, J=1.0 Hz, 2H), 4.26 (q, J=7.1 Hz, 2H), 3.45 (t, J=7.8 Hz, 2H), 1.27 (t, J=7.1 Hz, 3H), 0.80 (t, J=7.8 Hz, 2H), 0.06 (s, 8H).

### Synthesis of Intermediate C317

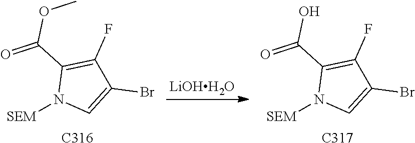

A solution of methyl 4-bromo-3-fluoro-1-{[2-(trimethylsilyl)ethoxy]methyl}pyrrole-2-carboxylate (3 g, 8.516 mmol, 1 equiv) and LiOH (1.02 g, 42.580 mmol, 5 equiv) in MeOH (10 mL), THF (10 mL), H₂O (10 mL) was stirred for 2 hr at 25° C. under N₂ atmosphere. The resulting mixture was extracted with EA (20 mL×2). The combined organic layers were washed with brine (10 mL×2), dried over anhydrous Na₂SO₄. After filtration, the filtrate was concentrated under reduced pressure to afford 4-bromo-3-fluoro-1-{[2-(trimethylsilyl)ethoxy]methyl}pyrrole-2-carboxylic acid (C317, 2.7 g, 93%) as a solid. LCMS (ES, m/z): 338 [M+H]⁺

### Synthesis of Intermediate C318

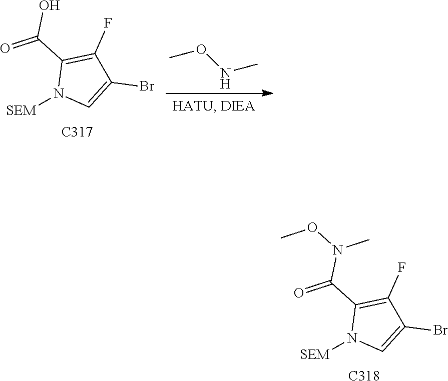

A solution 4-bromo-3-fluoro-1-{[2-(trimethylsilyl)ethoxy]methyl}pyrrole-2-carboxylic acid (2.7 g, 7.982 mmol, 1 equiv) and N,O-dimethylhydroxylamine (0.73 g, 11.973 mmol, 1.5 equiv), HATU (9.11 g, 23.946 mmol, 3 equiv), DIEA (3.10 g, 23.946 mmol, 3 equiv) in DCM (30 mL) was stirred for 2 hr at 25° C. under nitrogen atmosphere. The resulting mixture was extracted with EA (20 mL×2). The combined organic layers were washed with brine (10 mL×2), dried over anhydrous Na₂SO₄. After filtration, the filtrate was concentrated under reduced pressure. The residue was purified by silica gel column chromatography, eluted with PE:EA (0%˜50%) to afford 4-bromo-3-fluoro-N-methoxy-N-methyl-1-{[2-(trimethylsilyl) ethoxy] methyl}pyrrole-2-carboxamide (C318, 2.6 g, 85%) as a solid. ¹H NMR (300 MHz, DMSO-d6) δ 7.40 (d, J=4.5 Hz, 1H), 5.36 (d, J=1.1 Hz, 2H), 3.61 (s, 3H), 3.37 (d, J=8.0 Hz, 2H), 2.08 (s, OH), 0.79 (dd, J=8.9, 7.5 Hz, 2H).

### Synthesis of Intermediate C319

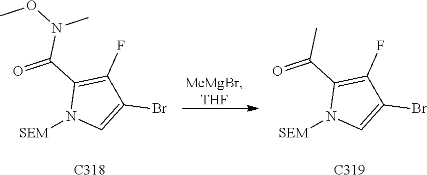

A mixture of 4-bromo-3-fluoro-N-methoxy-N-methyl-1-{[2-(trimethylsilyl)ethoxy]methyl}pyrrole-2-carboxamide (2.6 g, 6.818 mmol, 1 equiv) and MeMgBr (2.44 g, 20.454 mmol, 3 equiv) in THF (30 mL) was stirred for 12 h at 50° C. under N₂ atmosphere. The resulting mixture was extracted with EA (20 mL×2). The combined organic layers were washed with brine (10 mL×2), dried over anhydrous Na₂SO₄. After filtration, the filtrate was concentrated under reduced pressure. The residue was purified by silica gel column chromatography, eluted with PE:EA (0%˜50%) to afford 1-(4-bromo-3-fluoro-1-{[2-(trimethylsilyl)ethoxy]methyl}pyrrol-2-yl)ethanone (C319, 1.4 g, 61%) as a solid. LCMS (ES, m/z): 334 [M−H]⁻

### Synthesis of Intermediate C320

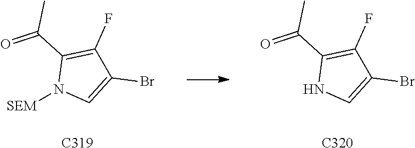

A solution of 1-(4-bromo-3-fluoro-1-{[2-(trimethylsilyl)ethoxy]methyl}pyrrol-2-yl)ethanone (1.4 g, 4.163 mmol, 1 equiv) and TFA (2.37 g, 20.815 mmol, 5 equiv) in DCM (14 mL) was stirred for 2 hr at 25° C. under N₂ atmosphere. The resulting mixture was concentrated under reduced pressure to afford 1-(4-bromo-3-fluoro-1H-pyrrol-2-yl)ethanone (C320, 700 mg, 81%) as a solid. ¹H NMR (300 MHz, DMSO-d6) δ 12.12 (s, 2H), 7.20 (d, J=4.2 Hz, 2H), 2.36 (d, J=2.5 Hz, 7H), 1.24 (s, 1H).

### Synthesis of Intermediate C321

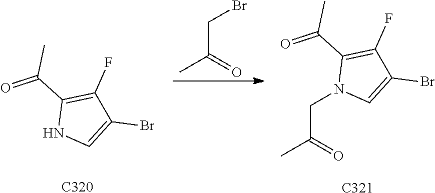

A solution of 1-(4-bromo-3-fluoro-1H-pyrrol-2-yl)ethanone (700 mg, 3.398 mmol, 1 equiv) and bromoacetone (698.13 mg, 5.097 mmol, 1.5 equiv) in ACN (7 mL) was stirred for 12 hr at 50° C. under N₂ atmosphere. The resulting mixture was extracted with EA (10 mL×2). The combined organic layers were washed with brine (5 mL×2), dried over anhydrous Na₂SO₄. After filtration, the filtrate was concentrated under reduced pressure The residue was purified by silica gel column chromatography, eluted with PE:EA (0%˜50%) to afford 1-(2-acetyl-4-bromo-3-fluoropyrrol-1-yl)propan-2-one (C321, 430 mg, 48%) as a solid. LCMS (ES, m/z): 262 [M+H]⁺

### Synthesis of Intermediate C322

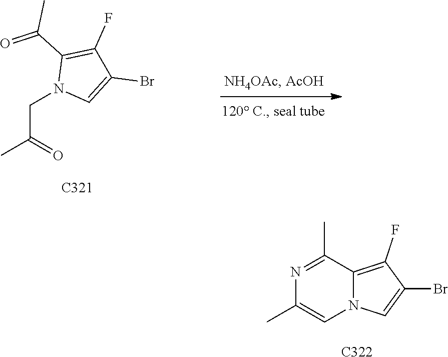

A solution 1-(2-acetyl-4-bromo-3-fluoropyrrol-1-yl)propan-2-one (430 mg, 1.641 mmol, 1 equiv) and NH₄OAc (2529.45 mg, 32.820 mmol, 20 equiv) in AcOH (4 mL, 69.806 mmol, 42.55 equiv) was stirred for 12 hr at 120° C. under N₂ atmosphere. The resulting mixture was extracted with EA (10 mL×2). The combined organic layers were washed with brine (5 mL×2), dried over anhydrous Na₂SO₄. After filtration, the filtrate was concentrated under reduced pressure. The residue was purified by silica gel column chromatography, eluted with PE:EA (0%˜50%) to afford 7-bromo-8-fluoro-1,3-dimethylpyrrolo[1,2-a]pyrazine (C322, 270 mg, 67%) as a solid. LCMS (ES, m/z): 243 [M+H]⁺

### Synthesis of Intermediate C323

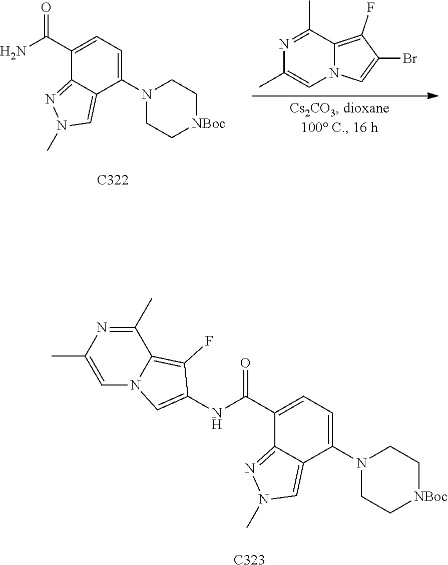

A mixture of tert-butyl 4-(7-carbamoyl-2-methylindazol-4-yl)piperazine-1-carboxylate (150 mg, 0.417 mmol, 1 equiv) and 7-bromo-8-fluoro-1,3-dimethylpyrrolo[1,2-a]pyrazine (121.73 mg, 0.500 mmol, 1.2 equiv), XantPhos (24.15 mg, 0.042 mmol, 0.1 equiv), Pd₂(dba)₃ (38.22 mg, 0.042 mmol, 0.1 equiv), Cs₂CO₃ (407.92 mg, 1.251 mmol, 3 equiv) in dioxane (3 mL) was stirred for 12 h at 100° C. under N₂ atmosphere. The resulting mixture was concentrated under reduced pressure. The residue was purified by silica gel column chromatography, eluted with PE:EA (0%˜80%) to afford tert-butyl 4-[7-({8-fluoro-1,3-dimethylpyrrolo[1,2-a]pyrazin-7-yl}carbamoyl)-2-methylindazol-4-yl]piperazine-1-carboxylate (C323, 40 mg, 18%) as a solid LCMS (ES, m/z): 522 [M+H]⁺

### Synthesis of Compound 483

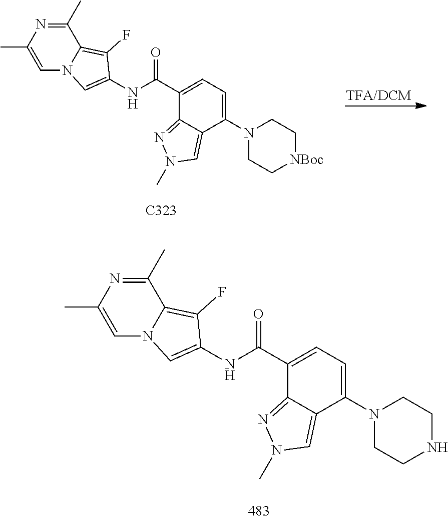

A mixture of tert-butyl 4-[7-({8-fluoro-1,3-dimethylpyrrolo[1,2-a]pyrazin-7-yl}carbamoyl)-2-methylindazol-4-yl]piperazine-1-carboxylate (40 mg, 0.077 mmol, 1 equiv) and TFA (43.72 mg, 0.385 mmol, 5 equiv) in DCM (2 mL) was stirred for 1 hr at 25° C. under N₂ atmosphere. The residue was purified by reverse flash chromatography (Condition 9, Gradient 1) to afford N-{8-fluoro-1,3-dimethylpyrrolo[1,2-a]pyrazin-7-yl}-2-methyl-4-(piperazin-1-yl) indazole-7-carboxamide (Compound 483, 3 mg, 9%) as a solid. LCMS (ES, m/z): 422 [M+H]^{+ 1}H NMR (300 MHz, DMSO-d6) δ 11.40 (s, 1H), 9.08 (s, 2H), 8.94 (s, 1H), 8.60 (s, 1H), 8.21 (s, 1H), 8.06 (d, J=7.8 Hz, 1H), 6.63 (d, J=8.0 Hz, 1H), 4.28 (s, 3H), 3.62 (s, 4H), 3.35 (s, 4H), 2.80 (s, 3H), 2.34 (s, 3H).

### Example 211: Synthesis of Compound 485

### Synthesis of Intermediate C234

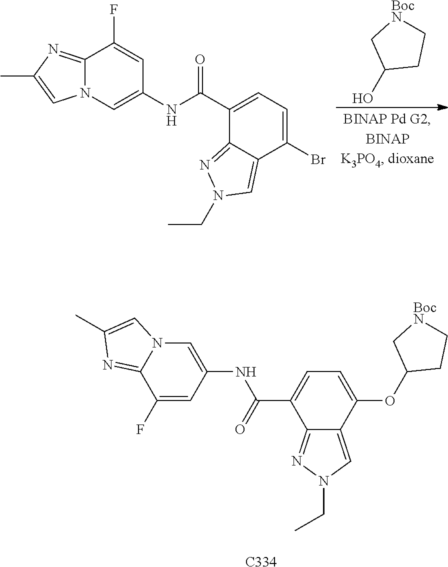

To a solution of 4-bromo-2-ethyl-N-{8-fluoro-2-methylimidazo[1,2-a]pyridin-6-yl}indazole-7-carboxamide (200 mg, 0.480 mmol, 1 equiv) and tert-butyl 3-hydroxypyrrolidine-1-carboxylate (269.8 mg, 1.440 mmol, 3.0 equiv) in dioxane (5 mL) were added K₃PO₄ (305.9 mg, 1.440 mmol, 3.0 equiv), BINAP (29.9 mg, 0.048 mmol, 0.1 equiv) and Binap Palladacycle Gen. 2 (44.8 mg, 0.048 mmol, 0.1 equiv). After stirring for 12 hr at 100° C. under a nitrogen atmosphere, the resulting mixture was concentrated under reduced pressure. The residue was purified by reverse flash chromatography (Condition 3, Gradient 1) to afford tert-butyl 3-{[2-ethyl-7-({8-fluoro-2-methylimidazo[1,2-a]pyridin-6-yl}carbamoyl) indazol-4-yl]oxy}pyrrolidine-1-carboxylate (C234, 80 mg, 31%) as a solid. LCMS (ES, m/z): 523 [M+H]⁺

### Synthesis of Compound 485

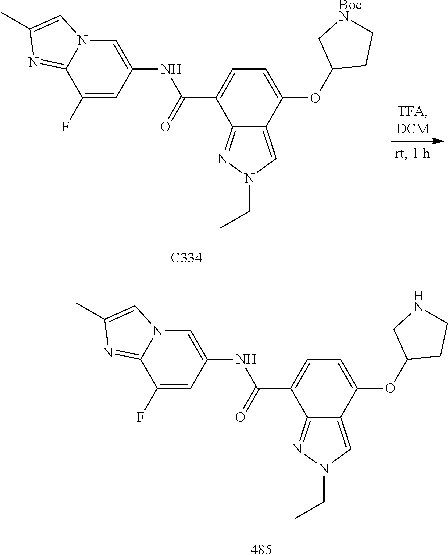 [SMILES: CCn1cc2c(OC3CCN(C(=O)OC(C)(C)C)C3)ccc(C(=O)Nc3cc(F)c4nc(C)cn4c3)c2n1 ; "C334" ; CCn1cc2c(OC3CCNC3)ccc(C(=O)Nc3cc(F)c4nc(C)cn4c3)c2n1 ; "485"]

A solution of tert-butyl 3-{[2-ethyl-7-({8-fluoro-2-methylimidazo[1,2-a]pyridin-6-yl}carbamoyl) indazol-4-yl]oxy}pyrrolidine-1-carboxylate (80 mg, 0.153 mmol, 1 equiv) and TFA (0.2 mL, 2.693 mmol) in DCM (2 mL) was stirred for 1 hr at room temperature. The resulting mixture was concentrated under reduced pressure. The residue was purified by reverse flash chromatography (Condition 6, Gradient 1) to afford 2-ethyl-N-{8-fluoro-2-methylimidazo[1,2-a]pyridin-6-yl}-4-(pyrrolidin-3-yloxy) indazole-7-carboxamide (Compound 485, 30 mg, 46%) as a solid. LCMS (ES, m/z): 423[M+H]^{+ 1}H NMR (300 MHz, DMSO-d₆) δ 11.07 (s, 1H), 9.41 (d, J=1.5 Hz, 1H), 9.29 (s, 1H), 9.13 (s, 1H), 8.68 (s, 1H), 8.10 (d, J=8.1 Hz, 2H), 7.71 (d, J=12.0 Hz, 1H), 6.75 (d, J=8.2 Hz, 1H), 5.43 (s, 1H), 4.64 (q, J=7.3 Hz, 2H), 3.65-3.35 (m, 4H), 3.51 (s, 1H), 2.43 (d, J=0.9 Hz, 3H), 2.41-2.20 (m, 2H), 1.62 (t, J=7.3 Hz, 3H).

### Example 212: Synthesis of Compound 486

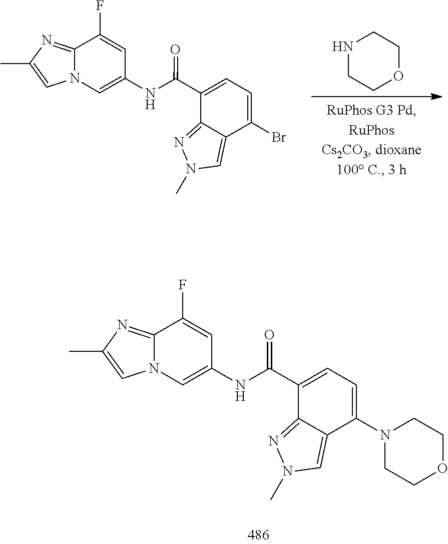 [SMILES: Cc1cn2cc(NC(=O)c3ccc(Br)c4cn(C)nc34)cc(F)c2n1 ; C1COCCN1 ; Cc1cn2cc(NC(=O)c3ccc(N4CCOCC4)c4cn(C)nc34)cc(F)c2n1]

To a stirred mixture of 4-bromo-N-{8-fluoro-2-methylimidazo[1,2-a]pyridin-6-yl}-2-methylindazole-7-carboxamide (100.0 mg, 0.24 mmol, 1.0 equiv) and morpholine (43.3 mg, 0.49 mmol, 2.0 equiv) in dioxane (1 mL) were added Cs₂CO₃ (243.0 mg, 0.74 mmol, 3.0 equiv), RuPhos (23.2 mg, 0.05 mmol, 0.2 equiv) and RuPhos Palladacycle Gen.3 (20.7 mg, 0.025 mmol, 0.1 equiv) at room temperature under nitrogen atmosphere. The resulting mixture was stirred for overnight at 100° C. under nitrogen atmosphere. The residue was purified by silica gel column chromatography, eluted with CH₂Cl₂/MeOH (10:1) to afford N-{8-fluoro-2-methylimidazo[1,2-a]pyridin-6-yl}-2-methyl-4-(morpholin-4-yl) indazole-7-carboxamide (Compound 486, 21 mg, 20%) as a solid. LCMS (ES, m/z): 409 [M+H]^{+ 1}H NMR (300 MHz, DMSO-d₆) δ 11.08 (s, 1H), 9.22 (d, J=1.6 Hz, 1H), 8.87 (s, 1H), 8.00 (d, J=8.0 Hz, 1H), 7.91 (d, J=2.7 Hz, 1H), 7.35 (dd, J=12.5, 1.7 Hz, 1H), 6.53 (d, J=8.1 Hz, 1H), 4.30 (s, 3H), 3.88-3.75 (m, 4H), 3.41 (t, J=4.8 Hz, 4H), 2.38-2.31 (m, 3H).

### Example 213: Synthesis of Compound 487

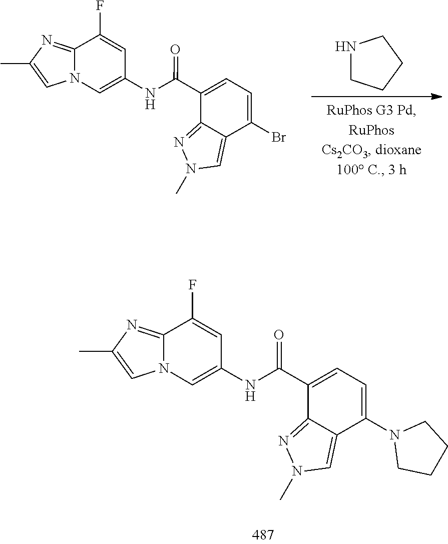 [SMILES: Cc1cn2cc(NC(=O)c3ccc(Br)c4cn(C)nc34)cc(F)c2n1 ; C1CCNC1 ; Cc1cn2cc(NC(=O)c3ccc(N4CCCC4)c4cn(C)nc34)cc(F)c2n1]

To a stirred mixture of 4-bromo-N-{8-fluoro-2-methylimidazo[1,2-a]pyridin-6-yl}-2-methylindazole-7-carboxamide (100.0 mg, 0.25 mmol, 1.0 equiv) and pyrrolidine (35.3 mg, 0.50 mmol, 2.0 equiv) in dioxane (1 mL) were added Cs₂CO₃ (243.0 mg, 0.74 mmol, 3.0 equiv), RuPhos (23.2 mg, 0.05 mmol, 0.2 equiv) and RuPhos Palladacycle Gen.3 (20.8 mg, 0.025 mmol, 0.1 equiv) at room temperature under nitrogen atmosphere. The resulting mixture was stirred for overnight at 100° C. under nitrogen atmosphere. The residue was purified by silica gel column chromatography, eluted with CH₂Cl₂/MeOH (10:1) to afford N-{8-fluoro-2-methylimidazo[1,2-a]pyridin-6-yl}-2-methyl-4-(pyrrolidin-1-yl) indazole-7-carboxamide (Compound 487, 47.5 mg, 48%) as a solid.

LCMS (ES, m/z): 393 [M+H]^{+ 1}H NMR (400 MHz, DMSO-d₆) δ 11.01 (s, 1H), 9.19 (d, J=1.7 Hz, 1H), 8.82 (s, 1H), 7.93 (d, J=8.3 Hz, 1H), 7.88 (d, J=3.1 Hz, 1H), 7.30 (dd, J=12.5, 1.7 Hz, 1H), 6.04 (d, J=8.4 Hz, 1H), 4.27 (s, 3H), 3.64 (t, J=4.9 Hz, 4H), 2.35 (s, 3H), 2.23-1.91 (m, 4H).

### Example 214: Synthesis of Compound 488

### Synthesis of Intermediate C235

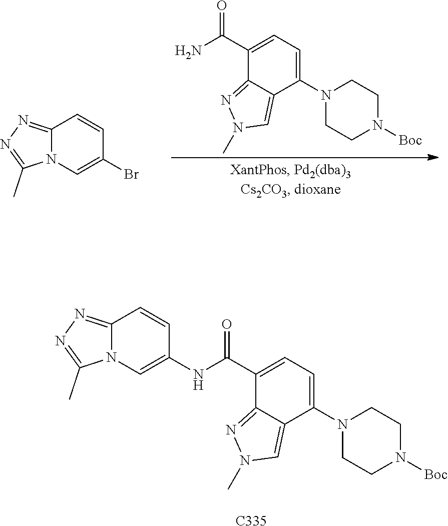

To a solution of 6-bromo-3-methyl-[1,2,4]triazolo[4,3-a]pyridine (100 mg, 0.472 mmol, 1 equiv) and tert-butyl 4-(7-carbamoyl-2-methylindazol-4-yl)piperazine-1-carboxylate (203.40 mg, 0.566 mmol, 1.2 equiv) in dioxane (4 mL) were added Cs₂CO₃ (460.9 mg, 1.416 mmol, 3.0 equiv), XantPhos (27.2 mg, 0.047 mmol, 0.1 equiv) and Pd₂(dba)₃ (43.1 mg, 0.047 mmol, 0.1 equiv). After stirring for 3 h at 100° C. under a nitrogen atmosphere, the resulting mixture was concentrated under reduced pressure. The residue was purified by reverse flash chromatography (Condition 3, Gradient 1) to afford in tert-butyl 4-[2-methyl-7-({3-methyl-[1,2,4]triazolo[4,3-a]pyridin-6-yl}carbamoyl) indazol-4-yl]piperazine-1-carboxylate (C235, 200 mg, 86%) as a solid. LCMS (ES, m/z): 491 [M+H]⁺

### Synthesis of Compound 488

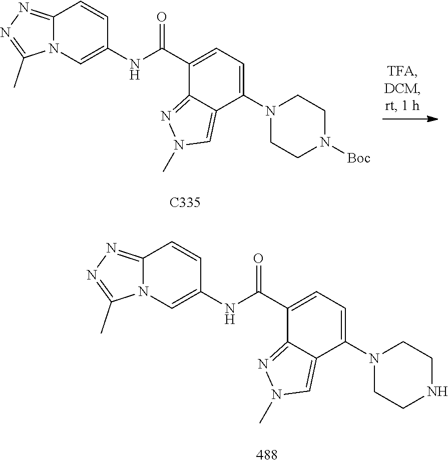

A solution of tert-butyl 4-[2-methyl-7-({3-methyl-[1,2,4]triazolo[4,3-a]pyridin-6-yl}carbamoyl) indazol-4-yl]piperazine-1-carboxylate (80 mg, 0.163 mmol, 1 equiv) and TFA (0.2 mL) in DCM (2 mL) was stirred for 1 hr at room temperature. The mixture was basified to pH 8 with NH₃(g) in methanol. The resulting mixture was concentrated under reduced pressure. The residue was purified by reverse flash chromatography (Condition 3, Gradient 5) to afford 2-methyl-N-{3-methyl-[1,2,4]triazolo[4,3-a]pyridin-6-yl}-4-(piperazin-1-yl) indazole-7-carboxamide (Compound 488, 30 mg, 47%) as a solid. LCMS (ES, m/z): 391[M+H]^{+ 1}H NMR (300 MHz, DMSO-d₆) δ 11.24 (s, 1H), 9.19-9.12 (m, 1H), 8.82 (s, 1H), 8.00 (d, J=8.1 Hz, 1H), 7.80 (dd, J=9.7, 1.0 Hz, 1H), 7.44 (dd, J=9.7, 1.8 Hz, 1H), 6.50 (d, J=8.2 Hz, 1H), 4.30 (s, 3H), 3.54 (s, 1H), 3.41-3.33 (m, 4H), 2.92 (d, J=5.6 Hz, 3H), 2.69 (s, 3H).

### Example 215: Synthesis of Compound 489

### Synthesis of Intermediate C236

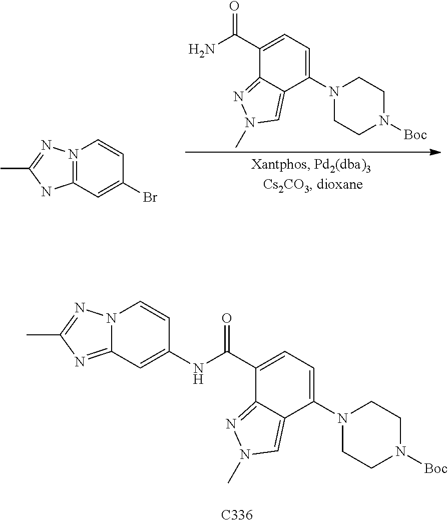

To a solution of 7-bromo-2-methyl-[1,2,4]triazolo[1,5-a]pyridine (100 mg, 0.472 mmol, 1 equiv) and tert-butyl 4-(7-carbamoyl-2-methylindazol-4-yl)piperazine-1-carboxylate (203.4 mg, 0.566 mmol, 1.2 equiv) in dioxane (4 mL) were added Cs₂CO₃ (460.9 mg, 1.416 mmol, 3.0 equiv), XantPhos (54.5 mg, 0.094 mmol, 0.2 equiv) and Pd₂(dba)₃ (27.1 mg, 0.047 mmol, 0.1 equiv). After stirring for 1 hr at 80° C. under nitrogen atmosphere, the resulting mixture was concentrated under reduced pressure. The residue was purified by reverse flash chromatography (Condition 3, Gradient 1) to afford tert-butyl 4-[2-methyl-7-({2-methyl-[1,2,4]triazolo[1,5-a]pyridin-7-yl}carbamoyl) indazol-4-yl]piperazine-1-carboxylate (C236, 200 mg, 86%) as a solid. LCMS (ES, m/z): 491 [M+H]⁺

### Synthesis of Compound 489

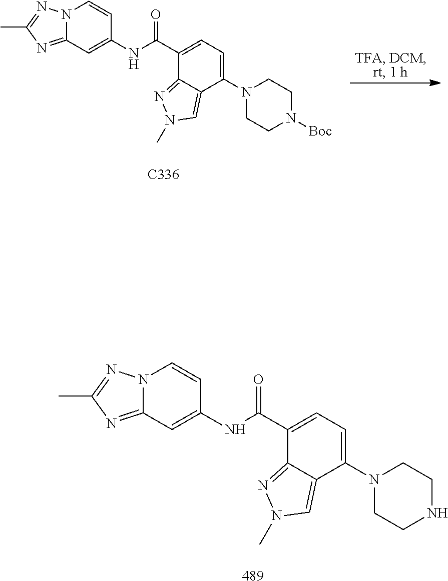

A solution of tert-butyl 4-[2-methyl-7-({2-methyl-[1,2,4]triazolo[1,5-a]pyridin-7-yl}carbamoyl) indazol-4-yl]piperazine-1-carboxylate (60 mg, 0.122 mmol, 1 equiv) and TFA (0.2 mL) in DCM (2 mL) was stirred for 1 hr at room temperature. The mixture was basified to pH 8 with NH₃(g) in methanol. The resulting mixture was concentrated under reduced pressure. The residue was purified by reverse flash chromatography (Condition 3, Gradient 5) to afford 2-methyl-N-{2-methyl-[1,2,4]triazolo[1,5-a]pyridin-7-yl}-4-(piperazin-1-yl) indazole-7-carboxamide (Compound 489, 20 mg, 41%) as a solid. LCMS (ES, m/z): 391[M+H]^{+ 1}H NMR (300 MHz, DMSO-d₆) δ 11.52 (s, 1H), 8.85-8.73 (m, 2H), 8.34-8.27 (m, 1H), 8.01 (d, J=8.2 Hz, 1H), 7.31 (dd, J=7.4, 2.3 Hz, 1H), 6.50 (d, J=8.2 Hz, 1H), 4.30 (s, 3H), 3.38 (t, J=4.9 Hz, 4H), 2.92 (t, J=4.9 Hz, 4H), 2.44 (s, 3H).

### Example 216: Synthesis of Compound 490

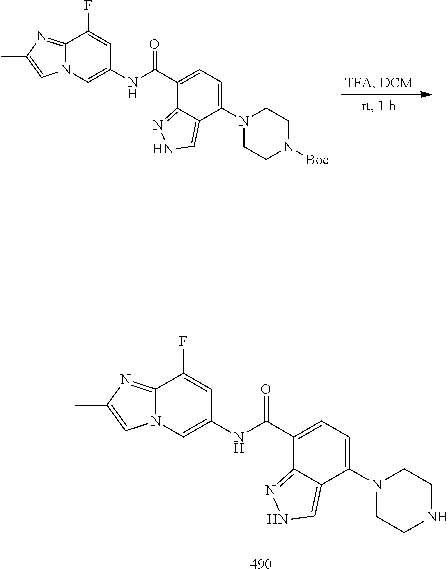 [SMILES: Cc1cn2cc(NC(=O)c3ccc(N4CCN(C(=O)OC(C)(C)C)CC4)c4c[nH]nc34)cc(F)c2n1 ; Cc1cn2cc(NC(=O)c3ccc(N4CCNCC4)c4c[nH]nc34)cc(F)c2n1 ; "490"]

To a stirred solution of tert-butyl 4-[7-({8-fluoro-2-methylimidazo[1,2-a]pyridin-6-yl}carbamoyl)-2H-indazol-4-yl]piperazine-1-carboxylate (60.0 mg, 0.122 mmol, 1 equiv) in DCM (2 mL) was added TFA (0.5 mL) dropwise at room temperature. The resulting mixture was stirred for 1 h at room temperature. The resulting mixture was concentrated under reduced pressure. The crude product was purified by Prep-HPLC (Condition 14, Gradient 2) to afford N-{8-fluoro-2-methylimidazo[1,2-a]pyridin-6-yl}-4-(piperazin-1-yl)-2H-indazole-7-carboxamide (Compound 490, 22.1 mg, 44%) as a solid. LCMS (ES, m/z): 394 [M+H]^{+ 1}H NMR (400 MHz, DMSO-d₆) δ 13.14 (m, 1H), 10.43 (m, 1H), 9.23-9.17 (m, 1H), 8.88 (s, 2H), 8.38 (s, 1H), 8.12-8.01 (m, 2H), 7.67 (s, 1H), 6.68 (d, J=8.2 Hz, 1H), 3.65 (t, J=5.1 Hz, 4H), 3.37 (m, 4H), 2.42 (s, 3H).

### Example 217: Synthesis of Compound 491

### Synthesis of Intermediate C237

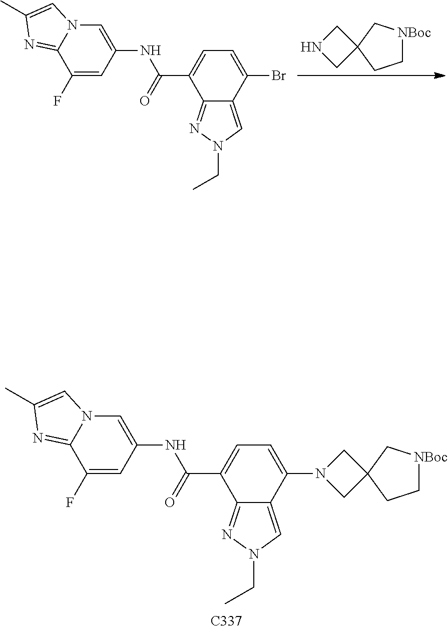

To a stirred solution of 4-bromo-2-ethyl-N-{8-fluoro-2-methylimidazo[1,2-a]pyridin-6-yl}indazole-7-carboxamide (140 mg, 0.336 mmol, 1 equiv), Cs₂CO₃ (329 mg, 1.008 mmol, 3 equiv) and tert-butyl 2,6-diazaspiro[3.4]octane-6-carboxylate (214 mg, 1.008 mmol, 3 equiv) in dioxane (2 mL) were added RuPhos (117 mg, 0.034 mmol, 0.1 equiv) and RuPhos Palladacycle Gen.3 (112 mg, 0.134 mmol, 0.4 equiv) at room temperature under nitrogen atmosphere. The resulting mixture was stirred for 3 hr at 100° C. The resulting mixture was concentrated under reduced pressure. The residue was purified by silica gel column chromatography, eluted with EA/MeOH (10:1) to afford tert-butyl 2-[2-ethyl-7-({8-fluoro-2-methylimidazo[1,2-a]pyridin-6-yl}carbamoyl) indazol-4-yl]-2,6-diazaspiro[3.4]octane-6-carboxylate (C237, 120 mg, 65%) as a solid. LCMS (ES, m/z): 548 [M+H]⁺

### Synthesis of Compound 491

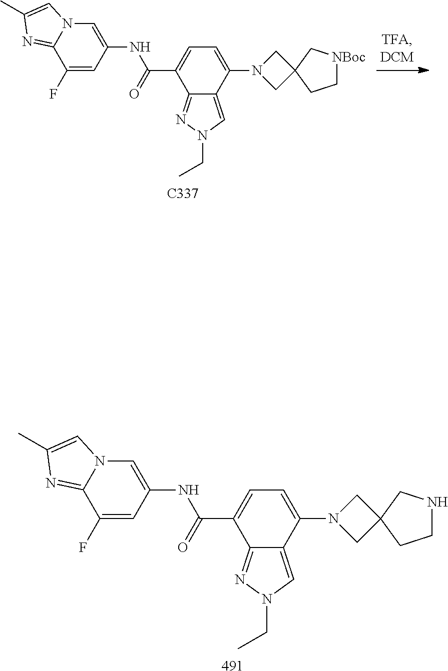

A solution of tert-butyl 2-[2-ethyl-7-({8-fluoro-2-methylimidazo[1,2-a]pyridin-6-yl}carbamoyl) indazol-4-yl]-2,6-diazaspiro[3.4]octane-6-carboxylate (120 mg, 0.219 mmol, 1 equiv) in trifluoroacetic acid (5 mL) and DCM (5 mL) was stirred for 2 hr at room temperature. The resulting mixture was concentrated under reduced pressure. The residue was basified to pH 8 with 7M NH₃(g) in MeOH. The resulting mixture was concentrated under vacuum. The residue was purified by reverse flash chromatography (Condition 3, Gradient 8) to afford 4-{2,6-diazaspiro[3.4]octan-2-yl}-2-ethyl-N-{8-fluoro-2-methylimidazo[1,2-a]pyridin-6-yl}indazole-7-carboxamide (491, 18 mg, 18%) as an solid. LCMS (ES, m/z): 448 [M+H]^{+ 1}H NMR (300 MHz, Chloroform-d) δ 11.00 (s, 1H), 9.26 (d, J=1.6 Hz, 1H), 8.20 (d, J=8.0 Hz, 1H), 8.00 (s, 1H), 7.47-7.39 (m, 1H), 6.83 (dd, J=11.3, 1.6 Hz, 1H), 5.96 (d, J=8.1 Hz, 1H), 4.53 (q, J=7.3 Hz, 2H), 4.23 (d, J=1.5 Hz, 4H), 3.27 (s, 2H), 3.13 (t, J=7.1 Hz, 2H), 2.49 (d, J=0.8 Hz, 3H), 2.18 (t, J=7.1 Hz, 2H), 1.73 (d, J=14.6 Hz, 3H).

### Example 218: Synthesis of Compound 492

### Synthesis of Intermediate C238

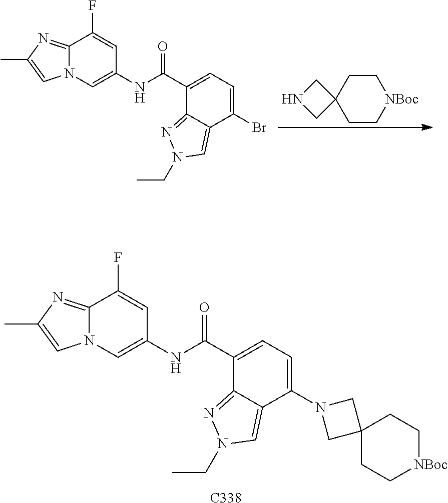

To a solution of 4-bromo-2-ethyl-N-{8-fluoro-2-methylimidazo[1,2-a]pyridin-6-yl}indazole-7-carboxamide (500 mg, 1.201 mmol, 1 equiv) and tert-butyl 2,7-diazaspiro[3.5]nonane-7-carboxylate (407.78 mg, 1.802 mmol, 1.5 equiv) in dioxane (12 mL) were added Cs₂CO₃ (1.17 g, 3.603 mmol, 3.0 equiv), RuPhos (56.1 mg, 0.120 mmol, 0.1 equiv) and RuPhos Palladacycle Gen.3 (200.9 mg, 0.240 mmol, 0.2 equiv). After stirring for 12 hr at 100° C. under nitrogen atmosphere, the resulting mixture was concentrated under reduced pressure. The resulting mixture was diluted with H₂O (20 mL). The resulting mixture was extracted with DCM (3×20 mL). The combined organic layers were washed with brine (1×20 mL), dried over anhydrous Na₂SO₄. After filtration, the filtrate was concentrated under reduced pressure. The residue was purified by reverse flash chromatography (Condition 5, Gradient 2) to afford tert-butyl 2-[2-ethyl-7-({8-fluoro-2-methylimidazo[1,2-a]pyridin-6-yl}carbamoyl) indazol-4-yl]-2,7-diazaspiro [3.5] nonane-7-carboxylate (C238, 500 mg, 74.11%) as a solid. LCMS (ES, m/z): 562 [M+H]⁺

### Synthesis of Compound 492

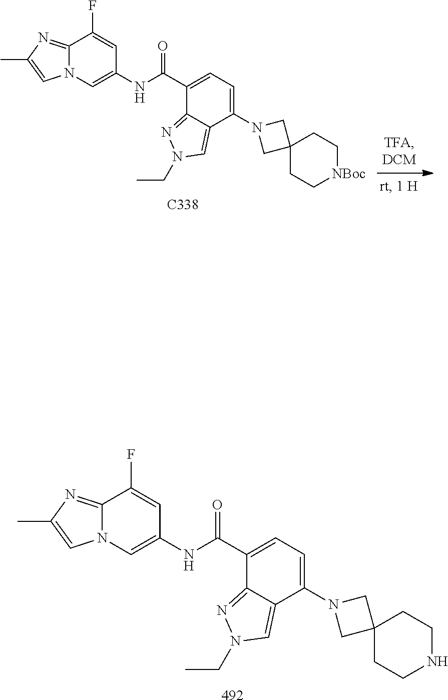

A solution of tert-butyl 2-[2-ethyl-7-({8-fluoro-2-methylimidazo[1,2-a]pyridin-6-yl}carbamoyl) indazol-4-yl]-2,7-diazaspiro[3.5]nonane-7-carboxylate (100 mg, 0.178 mmol, 1 equiv) and TFA (0.4 mL) in DCM (4 mL) was stirred for 1 hr at room temperature. The resulting mixture was concentrated under reduced pressure. The residue was purified by reverse flash chromatography (Condition 7, Gradient 3) to afford 4-{2,7-diazaspiro[3.5]nonan-2-yl}-2-ethyl-N-{8-fluoro-2-methylimidazo[1,2-a]pyridin-6-yl}indazole-7-carboxamide (Compound 492, 40 mg, 48%) as a solid. LCMS (ES, m/z): 462[M+H]^{+ 1}H NMR (300 MHz, DMSO-d₆) δ 10.96 (s, 1H), 9.19 (d, J=1.6 Hz, 1H), 8.71 (s, 1H), 7.97-7.86 (m, 2H), 7.27 (dd, J=12.4, 1.7 Hz, 1H), 5.88 (d, J=8.2 Hz, 1H), 4.57 (q, J=7.3 Hz, 2H), 3.97 (s, 4H), 2.69 (s, 4H), 2.35 (s, 3H), 1.72 (d, J=5.6 Hz, 4H), 1.61 (t, J=7.3 Hz, 3H).

### Example 219: Synthesis of Compound 493

### Synthesis of Intermediate C339

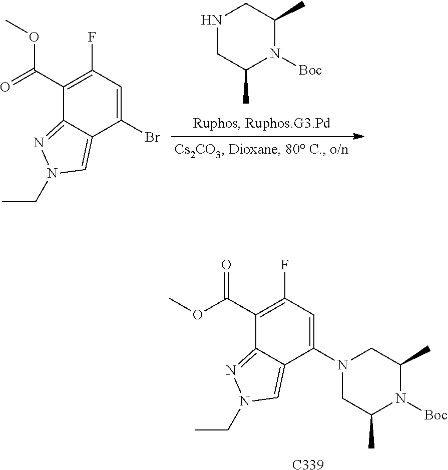 [SMILES: CCn1cc2c(Br)cc(F)c(C(=O)OC)c2n1 ; C[C@@H]1CNC[C@H](C)N1C(=O)OC(C)(C)C ; CCn1cc2c(N3C[C@@H](C)N(C(=O)OC(C)(C)C)[C@@H](C)C3)cc(F)c(C(=O)OC)c2n1 ; "C339"]

To a stirred mixture of methyl 4-bromo-2-ethyl-6-fluoroindazole-7-carboxylate (110 mg, 0.365 mmol, 1 equiv) and tert-butyl (2R,6S)-2,6-dimethylpiperazine-1-carboxylate (93.95 mg, 0.438 mmol, 1.2 equiv) in dioxane (2 mL) was added Cs₂CO₃ (357.07 mg, 1.095 mmol, 3 equiv), Ruphos (34.09 mg, 0.073 mmol, 0.2 equiv) and RuPhos Palladacycle Gen.3 (30.55 mg, 0.036 mmol, 0.1 equiv) at room temperature under nitrogen atmosphere. The resulting mixture was stirred overnight at 80° C. under nitrogen atmosphere. The resulting mixture was concentrated under reduced pressure. The residue was purified by silica gel column chromatography, eluted with PE/EA (1:1) to afford methyl 4-[(3R,5S)-4-(tert-butoxycarbonyl)-3,5-dimethylpiperazin-1-yl]-2-ethyl-6-fluoroindazole-7-carboxylate (C339, 95 mg, 59%) as a solid. LCMS (ES, m/z): 435 [M+H]⁺

### Synthesis of Intermediate C340

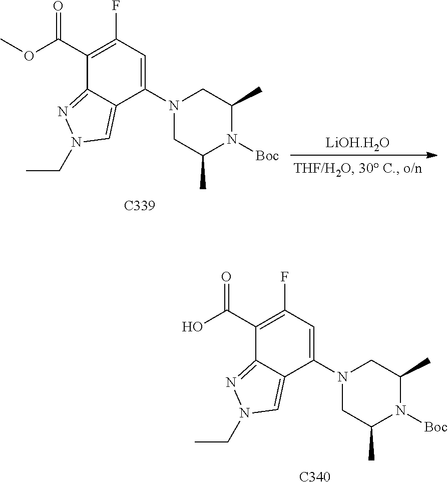 [SMILES: CCn1cc2c(N3C[C@@H](C)N(C(=O)OC(C)(C)C)[C@@H](C)C3)cc(F)c(C(=O)OC)c2n1 ; "LiOH.H2O" ; "C339" ; CCn1cc2c(N3C[C@@H](C)N(C(=O)OC(C)(C)C)[C@@H](C)C3)cc(F)c(C(=O)O)c2n1 ; "C340"]

To a stirred solution of methyl 4-[(3R,5S)-4-(tert-butoxycarbonyl)-3,5-dimethylpiperazin-1-yl]-2-ethyl-6-fluoroindazole-7-carboxylate (95 mg, 0.219 mmol, 1 equiv) in THF (1.2 mL) and H₂O (0.4 mL) was added lithiumol hydrate (18.35 mg, 0.438 mmol, 2 equiv) at room temperature. The resulting mixture was stirred overnight at 30° C. The resulting mixture was concentrated under vacuum. The resulting mixture was diluted with H₂O (5 mL). The mixture was acidified to pH 4 with citric acid and extracted with DCM (3×10 mL). The combined organic layers were washed with brine (1×10 mL), dried over anhydrous Na₂SO₄. After filtration, the filtrate was concentrated under reduced pressure to afford 4-[(3R,5S)-4-(tert-butoxycarbonyl)-3,5-dimethylpiperazin-1-yl]-2-ethyl-6-fluoroindazole-7-carboxylic acid (C340, 85 mg, 92%) as a solid. LCMS (ES, m/z): 419 [M−H]⁻

### Synthesis of Intermediate C341

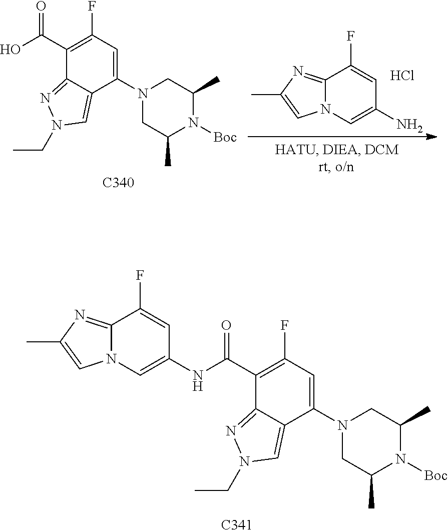

To a stirred mixture of 4-[(3R,5S)-4-(tert-butoxycarbonyl)-3,5-dimethylpiperazin-1-yl]-2-ethyl-6-fluoroindazole-7-carboxylic acid (85 mg, 0.202 mmol, 1 equiv) and 8-fluoro-2-methylimidazo[1,2-a]pyridin-6-amine hydrochloride (44.83 mg, 0.222 mmol, 1.10 equiv) in DCM (2 mL) was added DIEA (130.63 mg, 1.010 mmol, 5 equiv) and HATU (99.92 mg, 0.263 mmol, 1.3 equiv) at room temperature. The resulting mixture was stirred overnight at room temperature. The resulting mixture was concentrated under reduced pressure. The residue was purified by silica gel column chromatography, eluted with PE/EA (2:1) to afford tert-butyl (2R,6S)-4-[2-ethyl-6-fluoro-7-({8-fluoro-2-methylimidazo[1,2-a]pyridin-6-yl}carbamoyl) indazol-4-yl]-2,6-dimethylpiperazine-1-carboxylate (C341, 80 mg, 69%) as a solid.

LCMS (ES, m/z): 568 [M−H]⁻

### Synthesis of Compound 493

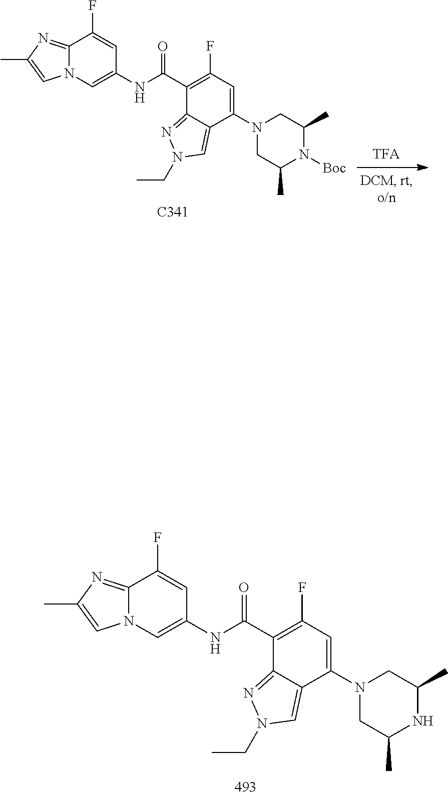

To a stirred solution of tert-butyl (2R,6S)-4-[2-ethyl-6-fluoro-7-({8-fluoro-2-methylimidazo[1,2-a]pyridin-6-yl}carbamoyl) indazol-4-yl]-2,6-dimethylpiperazine-1-carboxylate (80 mg, 0.141 mmol, 1 equiv) in DCM (1 mL) was added TFA (1 mL) at room temperature. The resulting mixture was stirred for 2 hr at room temperature. The resulting mixture was concentrated under reduced pressure. The crude product was purified by Prep-HPLC (Condition 15, Gradient 3) to afford 4-[(3R,5S)-3,5-dimethylpiperazin-1-yl]-2-ethyl-6-fluoro-N-{8-fluoro-2-methylimidazo[1,2-a]pyridin-6-yl}indazole-7-carboxamide; trifluoroacetic acid (Compound 493, 31.4 mg, 38%) as a solid. LCMS (ES, m/z): 468 [M+H]^{+ 1}H NMR (400 MHz, DMSO-d₆) δ 11.07 (s, 1H), 9.40 (s, 1H), 9.19 (s, 1H), 8.91 (s, 1H), 8.56 (d, J=11.5 Hz, 1H), 8.11 (s, 1H), 7.58 (d, J=12.2 Hz, 1H), 6.48 (d, J=14.4 Hz, 1H), 4.52 (q, J=7.3 Hz, 2H), 4.04 (d, J=13.2 Hz, 2H), 2.95 (t, J=12.4 Hz, 2H), 2.44 (d, J=4.9 Hz, 3H), 1.58 (t, J=7.3 Hz, 3H), 1.32 (d, J=6.4 Hz, 6H).

### Example 220: Synthesis of Compound 494

### Synthesis of Intermediate C342

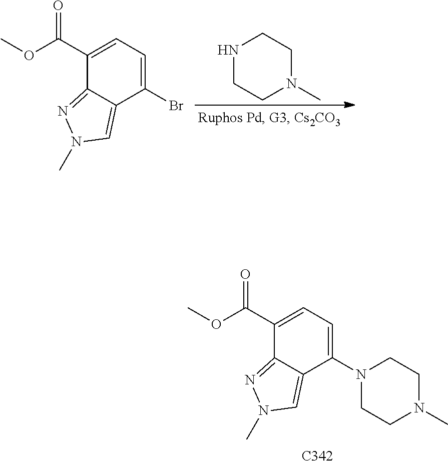

To a solution of methyl 4-bromo-2-methylindazole-7-carboxylate (3 g, 11.148 mmol, 1 equiv) and piperazine, 1-methyl- (1.68 g, 16.722 mmol, 1.5 equiv) in dioxane (5 mL) were added Cs₂CO₃ (9.08 g, 27.870 mmol, 2.5 equiv), RuPhos Palladacycle Gen.3 (932 mg, 1.115 mmol, 0.1 equiv) and RuPhos (520 mg, 1.115 mmol, 0.1 equiv). After stirring for 16 hr at 100° C. under a nitrogen atmosphere, the resulting mixture was concentrated under reduced pressure. The residue was purified by silica gel column chromatography, eluted with DCM:MeOH (10:1) to afford methyl 2-methyl-4-(4-methylpiperazin-1-yl) indazole-7-carboxylate (C342, 2.5 g, 77%) as an oil. LCMS (ES, m/z): 289 [M+H]⁺

### Synthesis of Intermediate C343

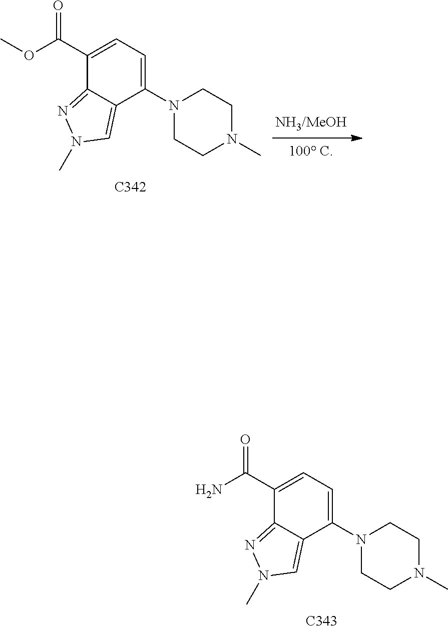

A solution of methyl 2-methyl-4-(4-methylpiperazin-1-yl) indazole-7-carboxylate (500 mg, 1.734 mmol, 1 equiv) in NH₃(g) in MeOH (40 mL) was stirred for 48 h at 100° C. The resulting mixture was concentrated under reduced pressure. The residue was purified by silica gel column chromatography, eluted with DCM:MeOH (10:1) to afford 2-methyl-4-(4-methylpiperazin-1-yl) indazole-7-carboxamide (400 mg, 84.39%) as a solid. LCMS (ES, m/z): 274 [M+H]⁺

### Synthesis of Intermediate C344

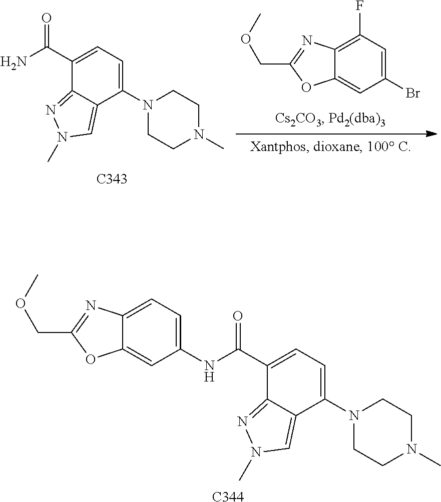

To a solution of 2-methyl-4-(4-methylpiperazin-1-yl) indazole-7-carboxamide (300 mg, 1.098 mmol, 1 equiv) and 6-bromo-4-fluoro-2-(methoxymethyl)-1,3-benzoxazole (428 mg, 1.647 mmol, 1.5 equiv) in dioxane (10 mL), were added Cs₂CO₃ (893.99 mg, 2.745 mmol, 2.5 equiv), Pd₂(dba)₃ (100 mg, 0.110 mmol, 0.1 equiv) and XantPhos (63 mg, 0.110 mmol, 0.1 equiv). After stirring for 4 h at 100° C. under a nitrogen atmosphere, the resulting mixture was concentrated under reduced pressure. The residue was purified by silica gel column chromatography, eluted with DCM:MeOH (10:1) to afford N-[4-fluoro-2-(methoxymethyl)-1,3-benzoxazol-6-yl]-2-methyl-4-(4-methylpiperazin-1-yl) indazole-7-carboxamide (C343, 200 mg, 40%) as a solid. ¹H NMR (400 MHz, DMSO-d₆) δ 11.24 (s, 1H), 8.80 (s, 1H), 7.96 (d, J=8.1 Hz, 1H), 7.41 (dd, J=12.2, 2.2 Hz, 1H), 7.24 (t, J=1.7 Hz, 1H), 6.49 (d, J=8.2 Hz, 1H), 4.72 (s, 2H), 4.28 (s, 3H), 3.91 (s, 3H), 3.42 (t, J=4.9 Hz, 4H), 2.54 (d, J=5.1 Hz, 4H), 2.27 (s, 3H).

### Synthesis of Compound 494

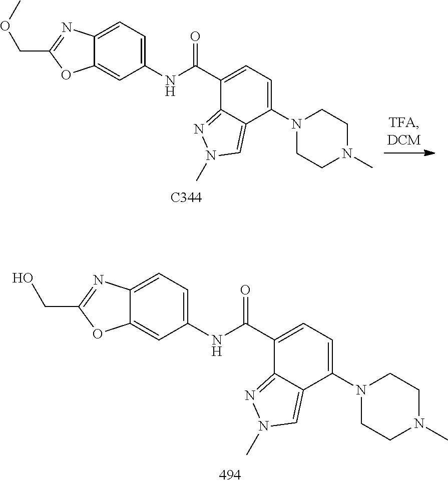

To a stirred solution of N-[4-fluoro-2-(methoxymethyl)-1,3-benzoxazol-6-yl]-2-methyl-4-(4-methylpiperazin-1-yl) indazole-7-carboxamide (150 mg, 0.331 mmol, 1 equiv) in DCM (5 mL) was added TFA (1 mL) dropwise at room temperature. The resulting mixture was stirred for 2 h at room temperature. The resulting mixture was concentrated under reduced pressure. The residue was purified by trituration with DMSO (5 mL) to afford N-[4-fluoro-2-(hydroxymethyl)-1,3-benzoxazol-6-yl]-2-methyl-4-(4-methylpiperazin-1-yl) indazole-7-carboxamide (Compound 494, 120 mg, 82%) as a solid. LCMS (ES, m/z): 439 [M+H]^{+ 1}H NMR (300 MHz, DMSO-d₆) δ 10.96 (s, 1H), 8.76 (s, 1H), 7.93 (d, J=8.0 Hz, 1H), 7.20 (dd, J=12.6, 2.3 Hz, 1H), 6.93 (t, J=1.9 Hz, 1H), 6.47 (d, J=8.2 Hz, 1H), 4.27 (s, 3H), 4.20 (s, 2H), 3.39 (t, J=5.0 Hz, 4H), 2.53 (d, J=5.6 Hz, 4H), 2.27 (s, 3H).

### Example 221: Synthesis of Compound 495

### Synthesis of Intermediate C345

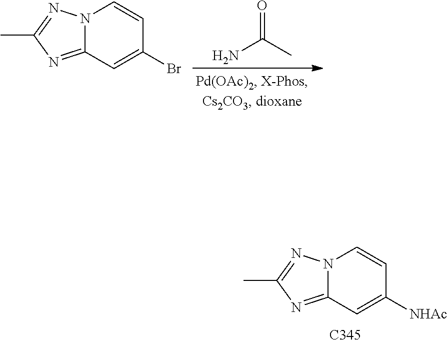

A solution of 7-bromo-2-methyl-[1,2,4]triazolo[1,5-a]pyridine (1.6 g, 7.545 mmol, 1 equiv) in dioxane (30 mL) was treated with acetamide (0.89 g, 15.090 mmol, 2 equiv), Pd(OAc)₂ (0.17 g, 0.755 mmol, 0.1 equiv) X-Phos (0.72 g, 1.509 mmol, 0.2 equiv), Cs₂CO₃ (4.92 g, 15.090 mmol, 2 equiv) for 8 hr at 100° C. under nitrogen atmosphere. The resulting mixture was washed with NaHCO₃ (3×100 mL). The residue was purified by silica gel column chromatography, eluted with PE:EA (50%) to afford N-{2-methyl-[1,2,4]triazolo[1,5-a]pyridin-7-yl}acetamide (2 g, 77%) as a solid. LCMS (ES, m/z): 191 [M+H]⁺

### Synthesis of Intermediate C346

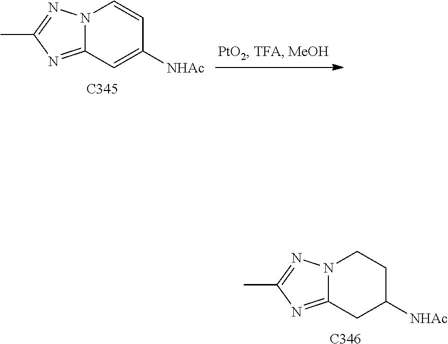

A solution of N-{2-methyl-[1,2,4]triazolo[1,5-a]pyridin-7-yl}acetamide (1.6 g, 8.412 mmol, 1 equiv) in EtOH (200 mL) was treated with PtO₂ (0.80 g, 3.533 mmol, 0.42 equiv), TFA (0.80 g, 6.982 mmol, 0.83 equiv) for 16 hr at 100° C. under H₂ (30 atm). The resulting mixture was filtered. the filter cake was washed with 100 mL of methanol. The filtrate was concentrated under reduced pressure. The residue was purified by silica gel column chromatography, eluted with EA/PE 1:3 to afford N-{2-methyl-5H,6H,7H,8H-[1,2,4]triazolo[1,5-a]pyridin-7-yl}acetamide (C346, 1 g, 61%, crude) as an oil. LCMS (ES, m/z): 195 [M+H]⁺

### Synthesis of Intermediate C347

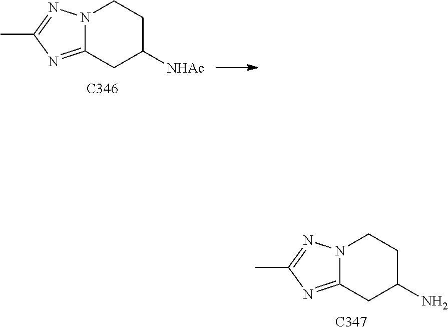

A solution of N-{2-methyl-5H,6H,7H,8H-[1,2,4]triazolo[1,5-a]pyridin-7-yl}acetamide (300 mg, 1.544 mmol, 1 equiv) in methanol (5 mL) was treated with HCl (5 mL, 15.000 mmol, 9.71 equiv) for 1 hr at 20° C. The residue was acidified to pH 7 with ammonia. The residue was purified by reverse flash chromatography (Condition 1, Gradient 1) to afford 2-methyl-5H,6H,7H,8H-[1,2,4]triazolo[1,5-a]pyridin-7-amine (C347, 200 mg, 85%) as a solid. LCMS (ES, m/z): 153 [M+H]⁺

### Synthesis of Intermediate C348

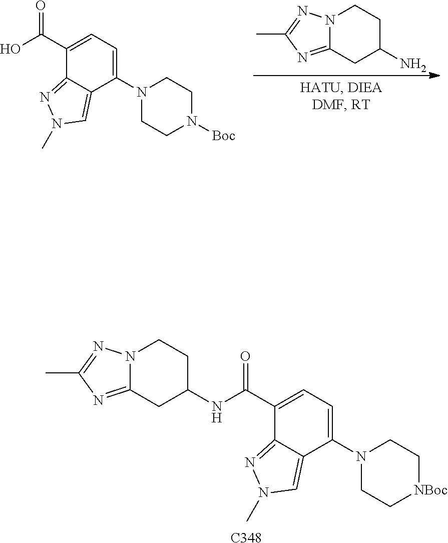

A solution of 4-[4-(tert-butoxycarbonyl)piperazin-1-yl]-2-methylindazole-7-carboxylic acid (100 mg, 0.277 mmol, 1.00 equiv) in DMF (5 mL) was treated with 2-methyl-5H,6H,7H,8H-[1,2,4]triazolo[1,5-a]pyridin-7-amine (42 mg, 0.277 mmol, 1.00 equiv), HATU (126 mg, 0.332 mmol, 1.20 equiv), DIEA (107 mg, 0.828 mmol, 2.99 equiv) for 2 hours at 20° C. The residue was purified by reverse flash chromatography (Condition 1, Gradient 1) to afford tert-butyl 4-[2-methyl-7-({2-methyl-5H,6H,7H,8H-[1,2,4]triazolo[1,5-a]pyridin-7-yl}carbamoyl) indazol-4-yl]piperazine-1-carboxylate-(C348, 60 mg, 43%) as a solid. LCMS (ES, m/z): 495 [M+H]⁺

### Synthesis of Compound 495

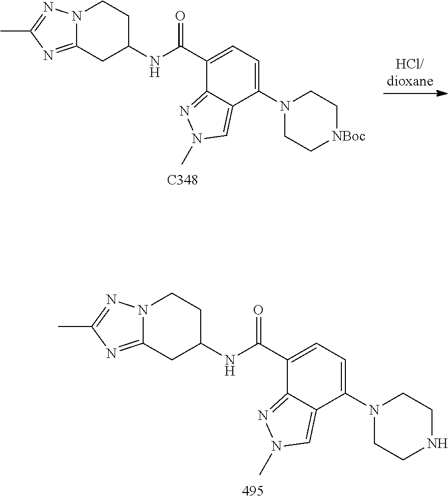

A solution of tert-butyl-4-[2-methyl-7-({2-methyl-5H,6H,7H,8H-[1,2,4]triazolo[1,5-a]pyridin-7-yl}carbamoyl) indazol-4-yl]piperazine-1-carboxylate (100 mg, 0.202 mmol, 1 equiv) in DCM (5 mL) was treated with HCl (gas) in 1,4-dioxane (10 mL, 329.128 mmol, 1627.87 equiv) for 30 mins at 20° C. Desired product could be detected by LCMS. The mixture was neutralized to pH 7 with NaHCO₃. The resulting solution was dried N₂ gas. The residue was purified by prep-(Condition 16, Gradient 1) to afford 2-methyl-N-{2-methyl-5H,6H,7H,8H-[1,2,4]triazolo[1,5-a]pyridin-7-yl}-4-(piperazin-1-yl) indazole-7-carboxamide (Compound 495, 6 mg, 7%) as a solid.

LCMS (ES, m/z): 395 [M+H]^{+ 1}H NMR (300 MHz, DMSO-d6) δ 9.21 (d, J=7.1 Hz, 1H), 8.67 (s, 1H), 7.87 (d, J=7.9 Hz, 1H), 6.41 (d, J=8.1 Hz, 1H), 4.52 (t, J=7.2 Hz, 1H), 4.20 (hept, J=7.6, 6.7 Hz, 2H), 4.11 (s, 3H), 3.26 (t, J=5.0 Hz, 5H), 3.17 (d, J=5.4 Hz, 2H), 2.91 (dt, J=9.5, 5.4 Hz, 4H), 2.55-2.47 (m, 1H), 2.39-2.23 (m, 1H), 2.28 (s, 1H), 2.21 (s, 3H).

### Example 222: Synthesis of Compound 435

### Synthesis of Intermediate C349

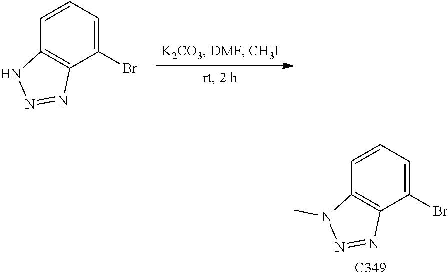

To a stirred solution of 4-bromo-2H-1,2,3-benzotriazole (2.7 g, 13.635 mmol, 1.0 equiv) and K₂CO₃ (3.8 g, 27.270 mmol, 2.0 equiv) in dimethylformamide (60 mL) were added methyl iodide (2.9 g, 20.453 mmol, 1.5 equiv) dropwise at 0° C. The resulting mixture was stirred for 1 h at room temperature. The resulting mixture was diluted with water (200 mL). The resulting mixture was extracted with ethyl acetate (2×200 mL). The combined organic layers were washed with water (2×200 mL), brine (2×200 mL) and dried over anhydrous Na₂SO₄. After filtration, the filtrate was concentrated under reduced pressure. The residue was purified by silica gel column chromatography, eluted with CH₂Cl₂/PE (5:1) to afford 4-bromo-1-methyl-1,2,3-benzotriazole (C349, 0.8 g, 25%) as a solid. LCMS (ES, m/z): 212 [M+H]⁺

### Synthesis of Intermediate C350

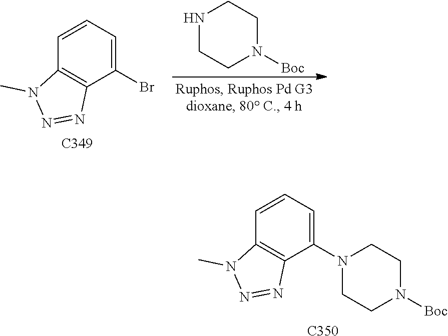

To a solution of 4-bromo-1-methyl-1,2,3-benzotriazole (0.8 g, 3.773 mmol, 1.0 equiv) and tert-butyl piperazine-1-carboxylate (0.9 g, 4.905 mmol, 1.3 equiv) in dioxane (10 mL) were added Cs₂CO₃ (3.0 g, 9.433 mmol, 2.5 equiv) and Ruphos (0.3 g, 0.755 mmol, 0.2 equiv), RuPhos Palladacycle Gen.3 (0.2 g, 0.377 mmol, 0.1 equiv). After stirring for 2 hr at 80° C. under a nitrogen atmosphere, the resulting mixture was concentrated under reduced pressure. The residue was purified by silica gel column chromatography, eluted with PE/EA (1:1) to afford tert-butyl 4-(1-methyl-1,2,3-benzotriazol-4-yl) piperazine-1-carboxylate (C350, 0.83 g, 63%) as a solid. LCMS (ES, m/z): 318 [M+H]⁺

### Synthesis of Intermediate C351

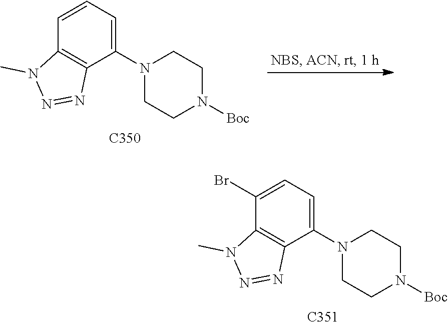 [SMILES: Cn1nnc2c(N3CCN(C(=O)OC(C)(C)C)CC3)cccc21 ; "C350" ; Cn1nnc2c(N3CCN(C(=O)OC(C)(C)C)CC3)ccc(Br)c21 ; "C351"]

To a stirred solution of tert-butyl 4-(1-methyl-1,2,3-benzotriazol-4-yl) piperazine-1-carboxylate (850.0 mg, 2.678 mmol, 1.0 equiv) in ACN (15 mL) were added NBS (524.3 mg, 2.946 mmol, 1.1 equiv) in portions at room temperature. The resulting mixture was stirred for 1 h at room temperature. The resulting mixture was diluted with deionized water (30 mL). The resulting mixture was extracted with ethyl acetate (2×40 mL). The combined organic layers were washed with water (2×30 mL), dried over anhydrous Na₂SO₄. After filtration, the filtrate was concentrated under reduced pressure. The residue was purified by silica gel column chromatography, eluted with PE/EA (1:1) to afford tert-butyl 4-(7-bromo-1-methyl-1,2,3-benzotriazol-4-yl) piperazine-1-carboxylate (C351 800 mg, 69%) as a solid. LCMS (ES, m/z): 396 [M+H]⁺

### Synthesis of Intermediate C352

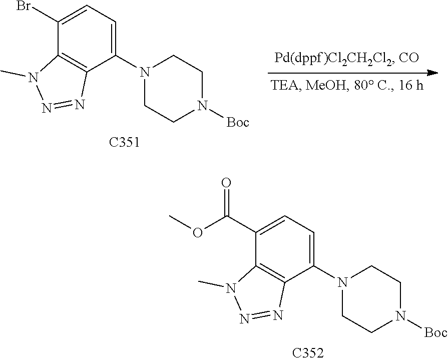

To a solution of tert-butyl 4-(7-bromo-1-methyl-1,2,3-benzotriazol-4-yl)piperazine-1-carboxylate (250.0 mg, 0.631 mmol, 1.0 equiv) in MeOH (20 mL) was added Pd(dppf)Cl₂ (46.1 mg, 0.063 mmol, 0.1 equiv), TEA (191.5 mg, 1.893 mmol, 3.0 equiv) in a pressure tank. The mixture was purged with nitrogen for 2 min and then was pressurized to 2 Mpa with carbon monoxide at 80° C. for 16 hr. The reaction mixture was cooled to room temperature and filtered to remove insoluble solids. The resulting mixture was concentrated under reduced pressure. The residue was purified by silica gel column chromatography, eluted with CH₂Cl₂/MeOH (30:1) to afford methyl 7-[4-(tert-butoxycarbonyl) piperazin-1-yl]-3-methyl-1,2,3-benzotriazole-4-carboxylate (240.0 mg, 94.24%) as a solid. LCMS (ES, m/z): 376 [M+H]⁺

### Synthesis of Intermediate C353

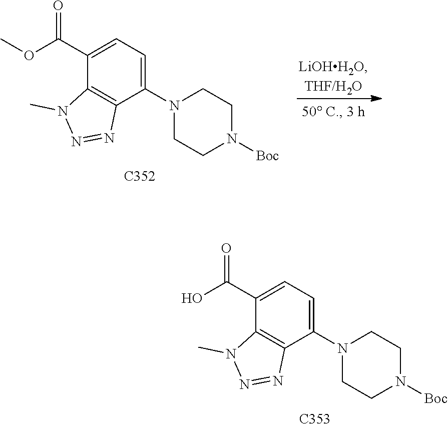

To a stirred mixture of methyl 7-[4-(tert-butoxycarbonyl) piperazin-1-yl]-3-methyl-1,2,3-benzotriazole-4-carboxylate (170.0 mg, 0.453 mmol, 1.0 equiv) in tetrahydrofuran (3 mL) and water (3 mL) was added LiOH·H₂O (108.4 mg, 4.530 mmol, 10.0 equiv) in portions at room temperature. The resulting mixture was stirred for 3 hr at 50° C. The resulting mixture was diluted with water (20 mL). The mixture was acidified to pH 6 with HCl (1 N). The resulting mixture was extracted with ethyl acetate (2×30 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 7-[4-(tert-butoxycarbonyl) piperazin-1-yl]-3-methyl-1,2,3-benzotriazole-4-carboxylic acid (C353, 150 mg, 85%) as a solid. LCMS (ES, m/z): 362 [M+H]⁺

### Synthesis of Intermediate C354

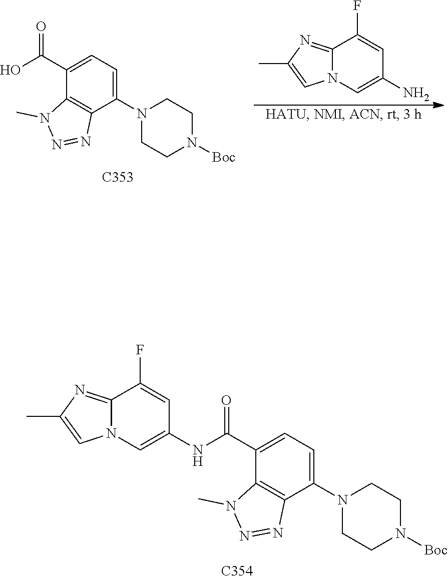

To a stirred solution of 7-[4-(tert-butoxycarbonyl) piperazin-1-yl]-3-methyl-1,2,3-benzotriazole-4-carboxylic acid (110.0 mg, 0.304 mmol, 1.0 equiv) and 8-fluoro-2-methylimidazo[1,2-a] pyridin-6-amine (75.4 mg, 0.456 mmol, 1.5 equiv) in ACN (3 mL) were added TCFH (111.0 mg, 0.395 mmol, 1.3 equiv) and NMI (64.8 mg, 0.790 mmol, 2.6 equiv) in portions at room temperature. The resulting mixture was stirred for 3 h at room temperature. The resulting mixture was extracted with ethyl acetate (2×20 mL). The combined organic layers were washed with brine (2×20 mL), dried over anhydrous Na₂SO₄. After filtration, the filtrate was concentrated under reduced pressure. This resulted in tert-butyl 4-[7-({8-fluoro-2-methylimidazo[1,2-a] pyridin-6-yl} carbamoyl)-1-methyl-1,2,3-benzotriazol-4-yl]piperazine-1-carboxylate (C354, 110 mg, 66%) as a solid. LCMS (ES, m/z): 509 [M+H]⁺

### Synthesis of Compound 435

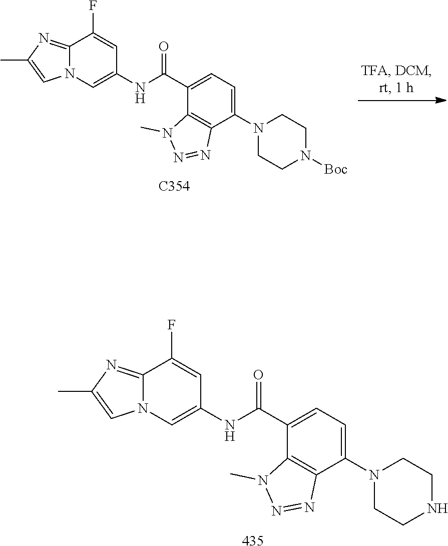

To a stirred solution of tert-butyl 4-[7-({8-fluoro-2-methylimidazo[1,2-a]pyridin-6-yl}carbamoyl)-1-methyl-1,2,3-benzotriazol-4-yl]piperazine-1-carboxylate (100.0 mg, 0.197 mmol, 1.0 equiv) in DCM (2 mL) was added TFA (0.5 mL) dropwise at room temperature. The resulting mixture was stirred for 1 hr at room temperature The resulting mixture was concentrated under reduced pressure. The crude product was purified by Prep-HPLC (Condition 13, Gradient 1) to afford N-{8-fluoro-2-methylimidazo[1,2-a] pyridin-6-yl}-3-methyl-7-(piperazin-1-yl)-1,2,3-benzotriazole-4-carboxamide (Compound 435, 26.8 mg, 32%) as a solid. LCMS (ES, m/z): 409 [M+H]^{+ 1}H NMR (300 MHz, DMSO-d₆) δ 10.87 (s, 1H), 9.18 (d, J=1.7 Hz, 1H), 8.00 (d, J=7.9 Hz, 1H), 7.93 (dd, J=3.2, 1.0 Hz, 1H), 7.39 (dd, J=12.4, 1.7 Hz, 1H), 7.24 (d, J=7.9 Hz, 1H), 4.58 (s, 3H), 3.04-2.97 (m, 8H), 2.36 (s, 3H).

### Example 223: Synthesis of Compound 436

### Synthesis of Intermediate C355

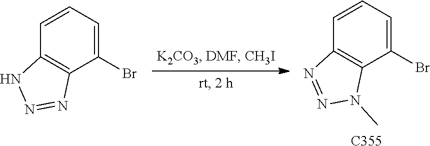

To a stirred solution of 4-bromo-2H-1,2,3-benzotriazole (2.7 g, 13.635 mmol, 1.0 equiv) and K₂CO₃ (3.8 g, 27.270 mmol, 2.0 equiv) in dimethylformamide (60 mL) were added methyl iodide (2.9 g, 20.453 mmol, 1.5 equiv) dropwise at 0° C. The resulting mixture was stirred for 2 h at room temperature. The resulting mixture was diluted with deionized water (100 mL). The resulting mixture was extracted with ethyl acetate (2×100 mL). The combined organic layers were washed with brine (2×100 mL), dried over anhydrous Na₂SO₄. After filtration, the filtrate was concentrated under reduced pressure. The residue was purified by silica gel column chromatography, eluted with CH₂Cl₂/PE (5:1) to afford 7-bromo-1-methyl-1,2,3-benzotriazole (C355, 1 g, 31%) as a solid. LCMS (ES, m/z): 212 [M+H]⁺

### Synthesis of Intermediate C356

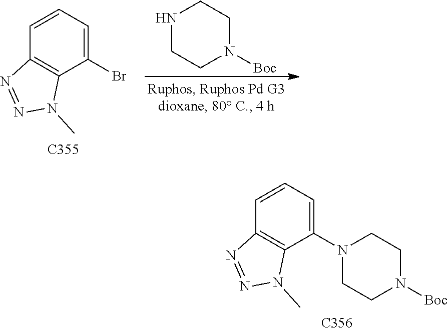

To a solution of 7-bromo-1-methyl-1,2,3-benzotriazole (0.8 g, 3.773 mmol, 1.0 equiv) and tert-butyl piperazine-1-carboxylate (0.91 g, 4.905 mmol, 1.3 equiv) in dioxane (10 mL) were added Cs₂CO₃ (3.1 g, 9.433 mmol, 2.5 equiv) and Ruphos (0.3 g, 0.755 mmol, 0.2 equiv), RuPhos Palladacycle Gen.3 (0.2 g, 0.377 mmol, 0.1 equiv). After stirring for 2 h at 80° C. under a nitrogen atmosphere, the resulting mixture was concentrated under reduced pressure. The residue was purified by silica gel column chromatography, eluted with PE/EA (1:1) to afford tert-butyl 4-(3-methyl-1,2,3-benzotriazol-4-yl) piperazine-1-carboxylate (C356, 780 mg, 61%) as a solid. LCMS (ES, m/z): 318 [M+H]⁺

### Synthesis of Intermediate C357

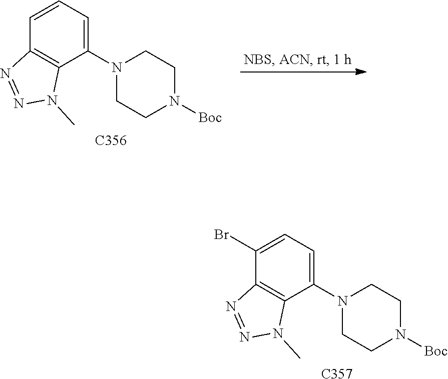

To a stirred solution of tert-butyl 4-(3-methyl-1,2,3-benzotriazol-4-yl) piperazine-1-carboxylate (850.0 mg, 2.678 mmol, 1.0 equiv) in ACN (15 mL) were added NBS (524.3 mg, 2.946 mmol, 1.1 equiv) in portions at room temperature. The resulting mixture was diluted with water (30 mL). The resulting mixture was extracted with ethyl acetate (2×40 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (1:1) to afford tert-butyl 4-(7-bromo-3-methyl-1,2,3-benzotriazol-4-yl)piperazine-1-carboxylate (C357, 830 mg, 72%) as a solid. LCMS (ES, m/z): 396 [M+H]⁺

### Synthesis of Intermediate C358

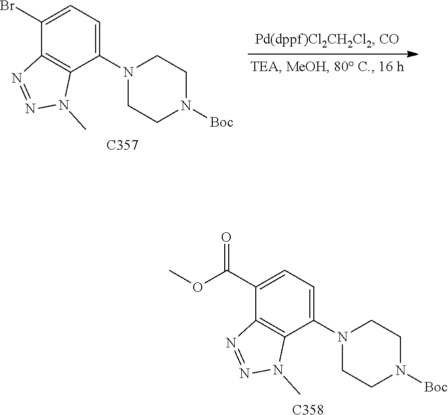

To a solution of tert-butyl 4-(7-bromo-3-methyl-1,2,3-benzotriazol-4-yl) piperazine-1-carboxylate (250 mg, 0.631 mmol, 1 equiv) in 20 mL MeOH was added Pd(dppf)Cl₂CH₂Cl₂ (46.1 mg, 0.063 mmol, 0.1 equiv), TEA (191.5 mg, 1.893 mmol, 3.0 equiv) in a pressure tank. The mixture was purged with nitrogen for 2 min and then was pressurized to 2 Mpa with carbon monoxide at 80° C. for 16 hr. The reaction mixture was cooled to room temperature and filtered to remove insoluble solids. The resulting mixture was concentrated under reduced pressure. The residue was purified by silica gel column chromatography, eluted with CH2Cl2/MeOH (30:1) to afford methyl 7-[4-(tert-butoxycarbonyl)piperazin-1-yl]-1-methyl-1,2,3-benzotriazole-4-carboxylate (C358, 230 mg, 87%) as a solid. LCMS (ES, m/z): 376 [M+H]⁺

### Synthesis of Intermediate C359

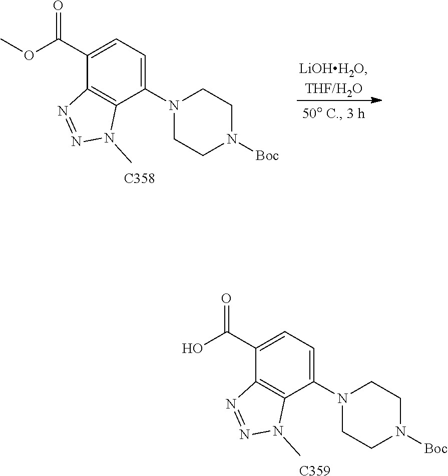

To a stirred mixture of methyl 7-[4-(tert-butoxycarbonyl) piperazin-1-yl]-1-methyl-1,2,3-benzotriazole-4-carboxylate (170.0 mg, 0.453 mmol, 1.0 equiv) in tetrahydrofuran (3 mL) and water (3 mL) was added LiOH·H₂O (108.4 mg, 4.530 mmol, 10.0 equiv) in portions at room temperature. The resulting mixture was stirred for 3 hr at 50° C. The resulting mixture was diluted with deionized water (20 mL). The mixture was acidified to pH 6 with HCl (1 N). The resulting mixture was extracted with ethyl acetate (2×30 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 7-[4-(tert-butoxycarbonyl) piperazin-1-yl]-1-methyl-1,2,3-benzotriazole-4-carboxylic acid (C359, 145 mg, 83%) as a solid. LCMS (ES, m/z): 362 [M+H]⁺

### Synthesis of Intermediate C360

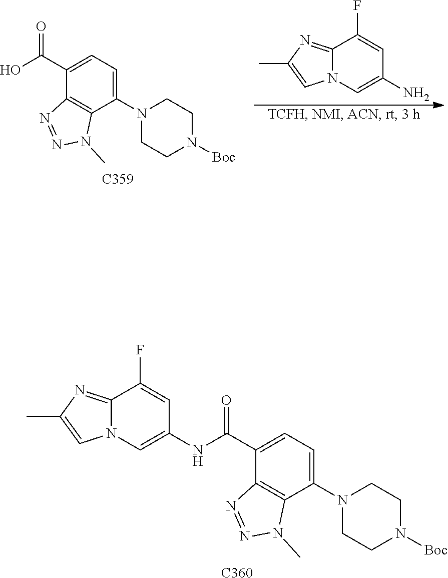

To a stirred solution of 7-[4-(tert-butoxycarbonyl) piperazin-1-yl]-1-methyl-1,2,3-benzotriazole-4-carboxylic acid (110.0 mg, 0.304 mmol, 1.0 equiv) and 8-fluoro-2-methylimidazo[1,2-a] pyridin-6-amine (75.4 mg, 0.456 mmol, 1.5 equiv) in ACN (3 mL) were added TCFH (111.0 mg, 0.395 mmol, 1.3 equiv) and NMI (64.8 mg, 0.790 mmol, 2.6 equiv) in portions at room temperature. The resulting mixture was stirred for 3 h at room temperature. The resulting mixture was extracted with ethyl acetate (2×20 mL). The combined organic layers were washed with brine (2×20 mL), dried over anhydrous Na₂SO₄. After filtration, the filtrate was concentrated under reduced pressure. This resulted in tert-butyl 4-[7-({8-fluoro-2-methylimidazo[1,2-a] pyridin-6-yl} carbamoyl)-3-methyl-1,2,3-benzotriazol-4-yl]piperazine-1-carboxylate (C360, 120 mg, 72%) as a solid. LCMS (ES, m/z): 509 [M+H]⁺

### Synthesis of Compound 436

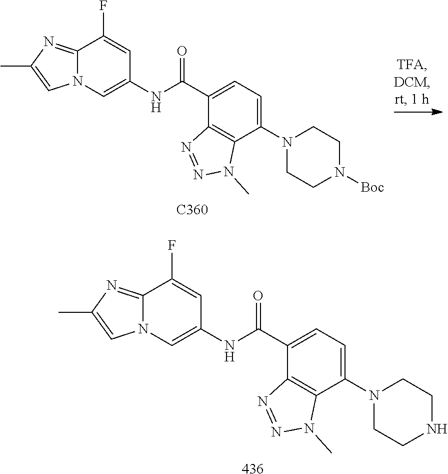

To a stirred solution of tert-butyl 4-[7-({8-fluoro-2-methylimidazo[1,2-a] pyridin-6-yl}carbamoyl)-3-methyl-1,2,3-benzotriazol-4-yl]piperazine-1-carboxylate (100.0 mg, 0.197 mmol, 1.0 equiv) in DCM (2 mL) was added TFA (0.5 mL) dropwise at room temperature. The resulting mixture was stirred for 1 h at room temperature. The resulting mixture was concentrated under reduced pressure. The crude product was purified by Prep-HPLC (Condition 13, Gradient 1) to afford N-{8-fluoro-2-methylimidazo[1,2-a]pyridin-6-yl}-1-methyl-7-(piperazin-1-yl)-1,2,3-benzotriazole-4-carboxamide (Compound 436, 41.4 mg, 51%) as a solid. LCMS (ES, m/z): 409 [M+H]^{+ 1}H NMR (300 MHz, DMSO-d₆) δ 10.53 (s, 1H), 9.14 (d, J=1.6 Hz, 1H), 7.98-7.91 (m, 1H), 7.75 (d, J=8.2 Hz, 1H), 7.27 (dd, J=12.7, 1.6 Hz, 1H), 6.67 (d, J=8.3 Hz, 1H), 4.31 (s, 3H), 3.80 (dd, J=6.1, 3.9 Hz, 4H), 2.94 (dd, J=6.2, 3.8 Hz, 4H), 2.35 (d, J=0.8 Hz, 3H).

### Example 224: Synthesis of Compound 448

### Synthesis of Intermediate C361

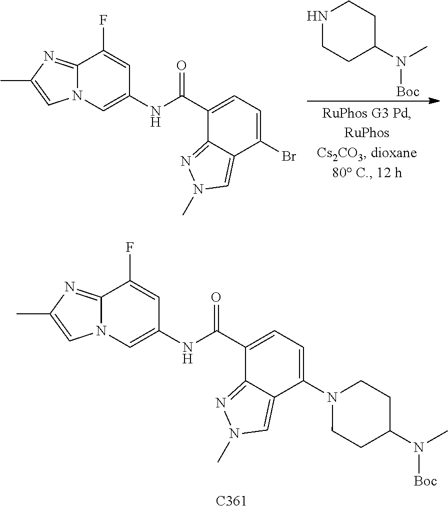

To a stirred mixture of 4-bromo-N-{8-fluoro-2-methylimidazo[1,2-a]pyridin-6-yl}-2-methylindazole-7-carboxamide (110 mg, 0.273 mmol, 1 equiv) and tert-butyl N-methyl-N-(piperidin-4-yl)carbamate (70.33 mg, 0.328 mmol, 1.2 equiv) in dioxane (2 mL) was added Cs₂CO₃ (267.31 mg, 0.819 mmol, 3 equiv), RuPhos (25.52 mg, 0.055 mmol, 0.2 equiv) and RuPhos Palladacycle Gen.3 (22.87 mg, 0.027 mmol, 0.1 equiv) at room temperature under nitrogen atmosphere. The resulting mixture was stirred for 12 hr at 80° C. under nitrogen atmosphere. The resulting mixture was concentrated under reduced pressure. The residue was purified by silica gel column chromatography, eluted with CH₂Cl₂/MeOH (10:1) to afford tert-butyl N-{1-[7-({8-fluoro-2-methylimidazo[1,2-a]pyridin-6-yl}carbamoyl)-2-methylindazol-4-yl]piperidin-4-yl}-N-methylcarbamate (C461, 80 mg, 54%) as a solid. LCMS (ES, m/z): 536 [M+H]⁺

### Synthesis of Compound 448

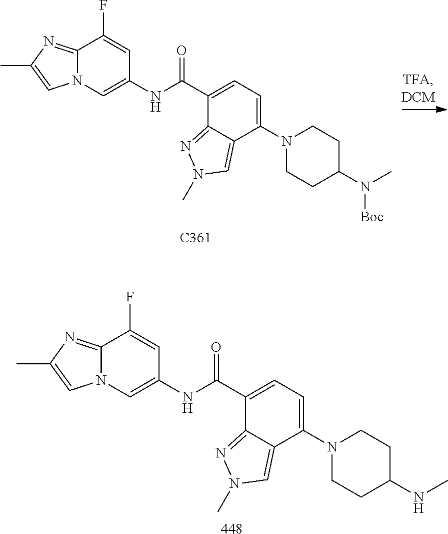

To a stirred solution of tert-butyl N-{1-[7-({8-fluoro-2-methylimidazo[1,2-a]pyridin-6-yl}carbamoyl)-2-methylindazol-4-yl]piperidin-4-yl}-N-methylcarbamate (80 mg, 0.149 mmol, 1 equiv) in DCM (1 mL) was added TFA (1 mL) at room temperature. The resulting mixture was stirred for 2 hr at room temperature. The resulting mixture was concentrated under reduced pressure. The crude product was purified by Prep-HPLC (Condition 10, Gradient 13) to afford N-{8-fluoro-2-methylimidazo[1,2-a]pyridin-6-yl}-2-methyl-4-[4-(methylamino)piperidin-1-yl]indazole-7-carboxamide (Compound 448, 29 mg, 44%) as a solid. LCMS (ES, m/z): 436 [M+H]^{+ 1}H NMR (400 MHz, DMSO-d₆) δ 11.06 (s, 1H), 9.21 (d, J=1.6 Hz, 1H), 8.77 (s, 1H), 7.97 (d, J=8.1 Hz, 1H), 7.90 (d, J=3.1 Hz, 1H), 7.34 (dd, J=12.4, 1.7 Hz, 1H), 6.49 (d, J=8.2 Hz, 1H), 4.30 (s, 3H), 3.88 (d, J=12.9 Hz, 2H), 3.13-3.02 (m, 2H), 2.61-2.52 (m, 1H), 2.35 (s, 3H), 2.33 (s, 3H), 2.01-1.93 (m, 2H), 1.41 (dd, J=16.9, 7.3 Hz, 2H).

### Example 225: Synthesis of Compound 449

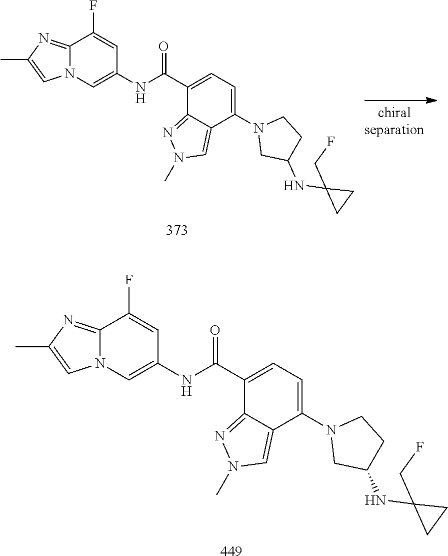 [SMILES: Cc1cn2cc(NC(=O)c3ccc(N4CCC(NC5(CF)CC5)C4)c4cn(C)nc34)cc(F)c2n1 ; "373" ; Cc1cn2cc(NC(=O)c3ccc(N4CC[C@H](NC5(CF)CC5)C4)c4cn(C)nc34)cc(F)c2n1 ; "449"]

Compound 373 (50 mg) was purified by Prep-Chiral HPLC (Condition 17, Gradient 1) to afford N-{8-fluoro-2-methylimidazo[1,2-a]pyridin-6-yl}-4-[(3S)-3-{[1-(fluoromethyl)cyclopropyl]amino}pyrrolidin-1-yl]-2-methylindazole-7-carboxamide (Compound 449, 15.1 mg, 29%, assumed) as a solid. LCMS (ES, m/z): 480 [M+H]^{+ 1}H NMR (400 MHz, Methanol-d₄) δ 9.05 (s, 1H), 8.55 (s, 1H), 8.04 (d, J=8.7 Hz, 1H), 7.72 (s, 1H), 7.24 (d, J=11.8 Hz, 1H), 6.07 (d, J=8.3 Hz, 1H), 4.52 (s, 1H), 4.40 (s, 1H), 4.30 (s, 3H), 3.90 (d, J=6.0 Hz, 3H), 3.84 (s, 1H), 3.72 (d, J=9.7 Hz, 1H), 2.44 (s, 3H), 2.36-2.31 (m, 1H), 2.05 (s, 1H), 0.82-0.70 (m, 4H).

### Example 226: Synthesis of Compound 461

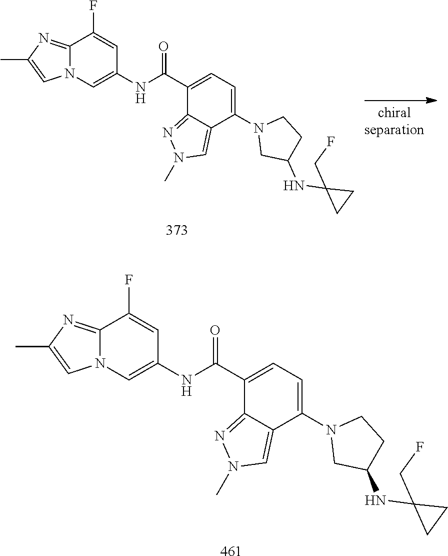 [SMILES: Cc1cn2cc(NC(=O)c3ccc(N4CCC(NC5(CF)CC5)C4)c4cn(C)nc34)cc(F)c2n1 ; "373" ; Cc1cn2cc(NC(=O)c3ccc(N4CC[C@@H](NC5(CF)CC5)C4)c4cn(C)nc34)cc(F)c2n1 ; "461"]

Compound 373 (50 mg) was purified by Prep-Chiral HPLC (Condition 17, Gradient 1) to afford N-{8-fluoro-2-methylimidazo[1,2-a]pyridin-6-yl}-4-[(3R)-3-{[1-(fluoromethyl)cyclopropyl]amino}pyrrolidin-1-yl]-2-methylindazole-7-carboxamide (Compound 461, 17.9 mg, 33%) as a solid. LCMS (ES, m/z): 480 [M+H]⁺1H NMR (400 MHz, Methanol-d₄) δ 9.02 (d, J=1.7 Hz, 1H), 8.51 (s, 1H), 8.03 (d, J=8.5 Hz, 1H), 7.70 (d, J=2.9 Hz, 1H), 7.18 (d, J=11.9 Hz, 1H), 6.04 (d, J=8.4 Hz, 1H), 4.52 (s, 1H), 4.40 (s, 1H), 4.29 (s, 3H), 3.94-3.76 (m, 3H), 3.69 (q, J=8.1 Hz, 1H), 3.47 (s, 1H), 2.44 (d, J=0.9 Hz, 3H), 2.33 (dd, J=12.1, 6.4 Hz, 1H), 2.04 (dd, J=12.4, 6.7 Hz, 1H), 0.85-0.66 (m, 4H).

### Example 227: Synthesis of Compounds

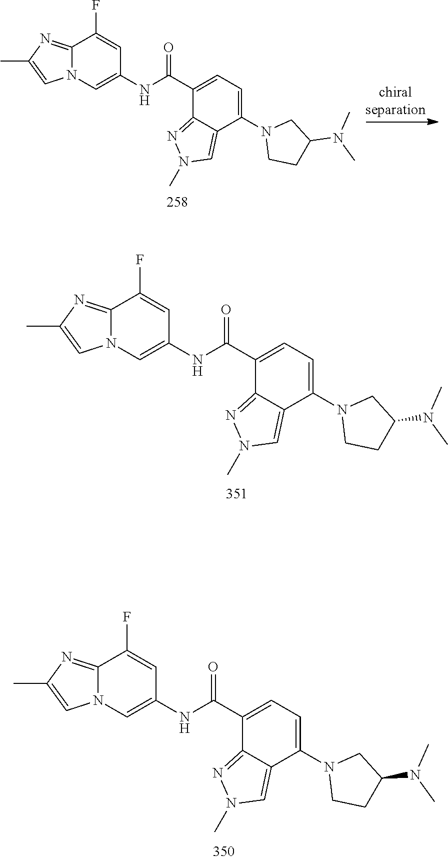

4-[3-(dimethylamino)pyrrolidin-1-yl]-N-{8-fluoro-2-methylimidazo[1,2-a]pyridin-6-yl}-2-methylindazole-7-carboxamide (20 mg) was purified by PREP-CHIRAL-HPLC with the following conditions (Column: CHIRAL ART Cellulose-SB, 2×25 cm, 5 um; Mobile Phase A: MtBE (0.1% DEA)-HPLC-Imported, Mobile Phase B: EtOH-HPLC; Flow rate: 20 mL/min; Gradient: 10% B to 10% B in 7.5 min; Wave Length: 220/254 nm; RT1 (min): 5.9; RT2 (min): 6.4; Sample Solvent: MeOH:DCM=2:1; Injection Volume: 0.2 mL; Number Of Runs: 18) to afford Compound 351 (5 mg) as a solid and Compound 350 (Second peak, 5 mg) as a solid. Compound 351: LCMS: (ES, m/z): 436 [M+H]^{+ 1}H NMR: (400 MHz, DMSO-d₆) δ 11.02 (d, J 2.4 Hz, 1H), 9.20 (t, J 2.1 Hz, 1H), 8.87 (d, J 2.4 Hz, 1H), 7.97-7.86 (m, 2H), 7.36-7.28 (m, 1H), 6.05 (dd, J 8.3, 2.4 Hz, 1H), 4.28 (d, J 2.4 Hz, 3H), 3.85 (s, 1H), 3.77 (s, 1H), 3.65 (d, J 9.3 Hz, 1H), 3.46 (s, 2H), 2.89 (s, 1H), 2.35 (d, J 2.4 Hz, 6H), 2.29 (s, 3H), 1.92 (s, 1H). Compound 350 LCMS: (ES, m/z): 436 [M+H]^{+ 1}H NMR: (400 MHz, DMSO-d₆) δ 11.02 (s, 1H), 9.20 (d, J 1.7 Hz, 1H), 8.87 (s, 1H), 7.96-7.86 (m, 2H), 7.31 (dd, J 12.4, 1.7 Hz, 1H), 6.05 (d, J 8.4 Hz, 1H), 4.28 (s, 3H), 3.85 (t, J 8.6 Hz, 1H), 3.77 (t, J 9.4 Hz, 1H), 3.64 (q, J 8.9 Hz, 1H), 3.48 (d, J 9.0 Hz, 2H), 2.91 (s, 1H), 2.37-2.33 (m, 3H), 2.29 (s, 6H), 1.92 (t, J 10.3 Hz, 1H).

### Example 228: Synthesis of Compound 304

### Synthesis of Intermediate C363

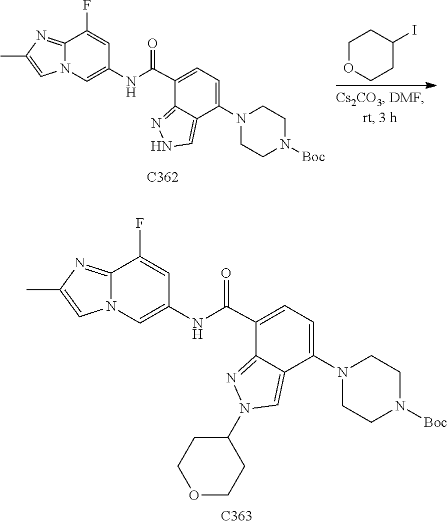

To a stirred mixture of tert-butyl 4-[7-({8-fluoro-2-methylimidazo[1,2-a]pyridin-6-yl}carbamoyl)-2H-indazol-4-yl]piperazine-1-carboxylate (100 mg, 0.20 mmol, 1.0 equiv) and 4-iodooxane (64.4 mg, 0.30 mmol, 1.5 equiv) in DMF (1 mL) were added Cs₂CO₃ (198.0 mg, 0.61 mmol, 3.0 equiv) at room temperature. The resulting mixture was stirred for 3 h at room temperature. The reaction was quenched by the addition of water at room temperature. The resulting mixture was extracted with EtOAc (3×5 mL). The combined organic layers were washed with brine (1×5 mL), dried over anhydrous Na₂SO₄. After filtration, the filtrate was concentrated under reduced pressure. The residue was purified by silica gel column chromatography, eluted with EA to afford tert-butyl 4-[7-({8-fluoro-2-methylimidazo[1,2-a]pyridin-6-yl}carbamoyl)-2-(oxan-4-yl) indazol-4-yl]piperazine-1-carboxylate (41 mg, 35%) as a solid. LCMS (ES, m/z): 578 [M+H]⁺

### Synthesis of Compound 304

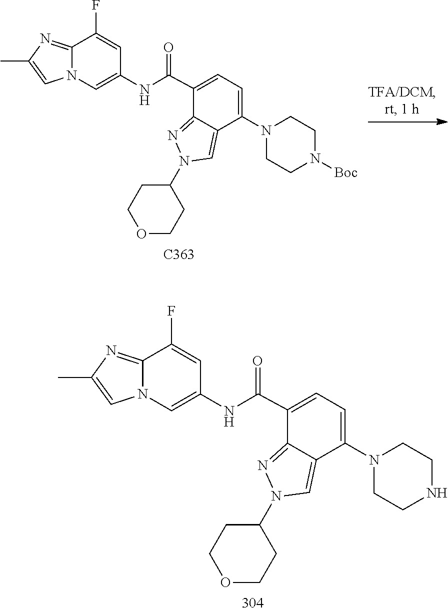

To a stirred mixture of tert-butyl 4-[7-({8-fluoro-2-methylimidazo[1,2-a]pyridin-6-yl}carbamoyl)-2-(oxan-4-yl) indazol-4-yl]piperazine-1-carboxylate (40 mg, 0.07 mmol, 1.0 equiv) in DCM (0.4 mL) was added TFA (0.4 mL) at room temperature. The resulting mixture was stirred for 1 h at room temperature. The resulting mixture was concentrated under reduced pressure. The crude product was purified by Prep-HPLC (Condition 14, Gradient 1) to afford N-{8-fluoro-2-methylimidazo[1,2-a]pyridin-6-yl}-2-(oxan-4-yl)-4-(piperazin-1-yl) indazole-7-carboxamide trifluoroacetic acid salt (22.8 mg, 50%) as a solid. LCMS (ES, m/z): 478 [M+H]^{+ 1}H NMR (300 MHz, DMSO-d₆) δ 11.17 (s, 1H), 9.44 (d, J=1.6 Hz, 1H), 9.15 (s, 2H), 9.00 (s, 1H), 8.13 (d, J=2.6 Hz, 1H), 8.04 (d, J=8.0 Hz, 1H), 7.71 (dd, J=11.8, 1.6 Hz, 1H), 6.62 (d, J=8.1 Hz, 1H), 4.95 (tt, J=10.4, 5.5 Hz, 1H), 4.10 (dt, J=11.2, 3.2 Hz, 2H), 3.69-3.51 (m, 6H), 3.37-3.35 (m, 4H), 2.44 (s, 3H), 2.25 (td, J=10.3, 9.5, 4.1 Hz, 4H).

### Example 229: Synthesis of Compound 342

### Synthesis of Intermediate C365

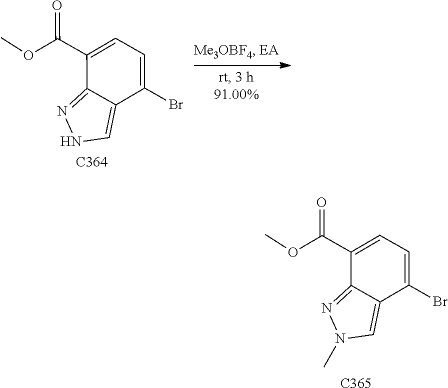

To a stirred mixture of methyl 4-bromo-2H-indazole-7-carboxylate (5.0 g, 19.602 mmol, 1 equiv) and in EA (150 mL) was added tetrafluoroboranuide; trimethyloxidanium (14.50 g, 98.010 mmol, 5 equiv) at room temperature. The resulting mixture was stirred for 3 h at room temperature. The resulting mixture was diluted with EA (150 mL) and washed with water (3×200 mL). The organic phase was dried over anhydrous Na₂SO₄. After filtration, the filtrate was concentrated under reduced pressure to afford methyl 4-bromo-2-methylindazole-7-carboxylate (4.8 g, 91%) as a solid. LCMS (ES, m/z): 269[M+H]^{+ 1}H NMR (300 MHz, DMSO-d₆) δ 8.62 (s, 1H), 7.84 (d, J=7.6 Hz, 1H), 7.42 (d, J=7.7 Hz, 1H), 4.25 (s, 3H), 3.89 (s, 3H).

### Synthesis of Intermediate C366

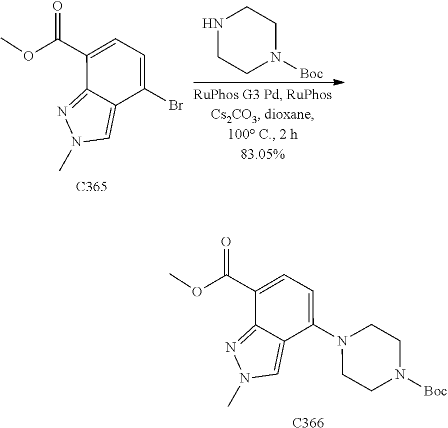

To a stirred mixture of methyl 4-bromo-2-methylindazole-7-carboxylate (4.5 g, 16.723 mmol, 1 equiv) and tert-butyl piperazine-1-carboxylate (6.23 g, 33.446 mmol, 2 equiv) in dioxane (90 mL) was added Cs₂CO₃ (16.35 g, 50.169 mmol, 3 equiv), RuPhos (1.56 g, 3.345 mmol, 0.2 equiv) and RuPhos Palladacycle Gen.3 (1.40 g, 1.672 mmol, 0.1 equiv) at room temperature under nitrogen atmosphere. The resulting mixture was stirred for 2 h at 100° C. under nitrogen atmosphere. The resulting mixture was concentrated under vacuum. The residue was purified by silica gel column chromatography, eluted with PE/EA (1:1) to afford methyl 4-[4-(tert-butoxycarbonyl)piperazin-1-yl]-2-methylindazole-7-carboxylate (5.2 g, 83%) as a solid. LCMS (ES, m/z): 375[M+H]⁺

### Synthesis of Intermediate C367

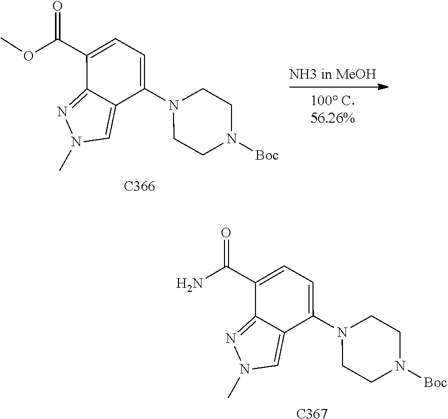

A solution of methyl 4-[4-(tert-butoxycarbonyl)piperazin-1-yl]-2-methylindazole-7-carboxylate (2.5 g, 6.677 mmol, 1 equiv) in NH₃(g) in MeOH (70 mL) was stirred for 2 days at 100° C. The resulting mixture was concentrated under reduced pressure. The residue was purified by silica gel column chromatography, eluted with PE/EA (1:1) to afford tert-butyl 4-(7-carbamoyl-2-methylindazol-4-yl)piperazine-1-carboxylate (1.35 g, 56%) as an solid. LCMS (ES, m/z): 360[M+H]⁺

### Synthesis of Intermediate C368

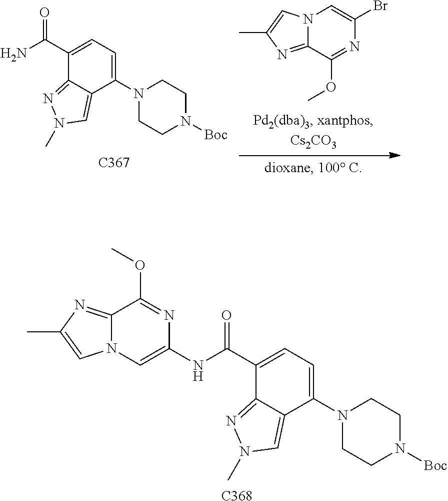

To a solution of tert-butyl 4-(7-carbamoyl-2-methylindazol-4-yl)piperazine-1-carboxylate (60 mg, 0.167 mmol, 1 equiv) and 6-bromo-8-methoxy-2-methylimidazo[1,2-a]pyrazine (48.49 mg, 0.200 mmol, 1.2 equiv) in dioxane (2 mL) and Cs₂CO₃ (108.78 mg, 0.334 mmol, 2 equiv) were added Xantphos (19.32 mg, 0.033 mmol, 0.2 equiv) and Pd₂(dba)₃ (15.29 mg, 0.017 mmol, 0.1 equiv). After stirring for overnight at 100° C. under a nitrogen atmosphere, the resulting mixture was concentrated under reduced pressure. The residue was purified by silica gel column chromatography, eluted with PE/EA (1:2) to afford tert-butyl 4-[7-({8-methoxy-2-methylimidazo[1,2-a]pyrazin-6-yl}carbamoyl)-2-methylindazol-4-yl]piperazine-1-carboxylate (35 mg, 40%) as a solid. LCMS (ES, m/z): 521 [M+H]⁺

### Synthesis of Compound 342

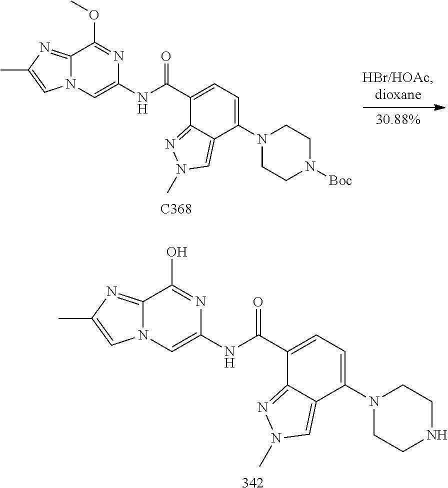

A solution of tert-butyl 4-[7-({8-methoxy-2-methylimidazo[1,2-a]pyrazin-6-yl}carbamoyl)-2-methylindazol-4-yl]piperazine-1-carboxylate (20 mg, 0.038 mmol, 1 equiv) in 1,4-dioxane was treated with HBr in AcOH (0.5 mL, 17.117 mmol, 445.56 equiv) for 2 h at 80° C. The resulting mixture was concentrated under reduced pressure. The crude product was purified by Prep-HPLC (Condition 17, Gradient 1) to afford N-{8-hydroxy-2-methylimidazo[1,2-a]pyrazin-6-yl}-2-methyl-4-(piperazin-1-yl) indazole-7-carboxamide (6.5 mg, 31%) as a solid. LCMS (ES, m/z): 407 [M+H]^{+ 1}H NMR (400 MHz, DMSO-d₆) δ 10.94 (s, 1H), 8.90 (s, 3H), 8.33 (s, 1H), 8.01 (d, J=8.0 Hz, 1H), 7.78 (s, 1H), 6.61 (d, J=8.1 Hz, 1H), 4.30 (s, 3H), 3.62 (t, 4H), 3.35 (t, 4H), 2.34 (s, 3H). ¹⁹F NMR (400 MHz, DMSO-d₆) δ −73.89

### Example 230: Synthesis of Compound 393

### Synthesis of Intermediate C370

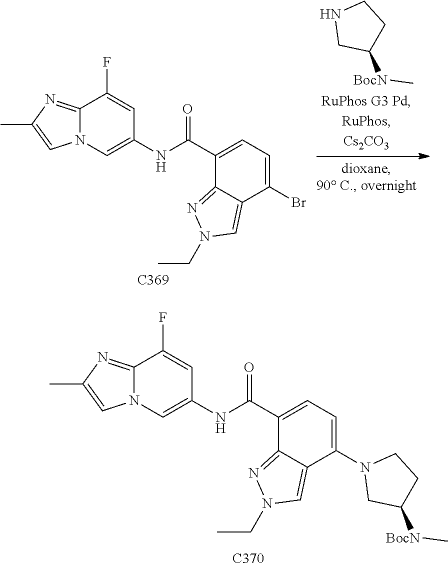

To a stirred solution of 4-bromo-2-ethyl-N-{8-fluoro-2-methylimidazo[1,2-a]pyridin-6-yl}indazole-7-carboxamide (180 mg, 0.43 mmol, 1 equiv) and tert-butyl (R)-methyl(pyrrolidin-3-yl)carbamate (87 mg, 0.43 mmol, 1 equiv) in dioxane (10 mL) were added Cs₂CO₃ (423 mg, 1.29 mmol, 3 equiv), RuPhos (41 mg, 0.086 mmol, 0.2 equiv) and RuPhos Palladacycle Gen.3 (36 mg, 0.043 mmol, 0.1 equiv) in portions at room temperature under nitrogen atmosphere. The resulting mixture was stirred for overnight at 90° C. under nitrogen atmosphere. The resulting mixture was concentrated under reduced pressure. The resulting mixture was added H₂O (20 mL) and extracted with EtOAc (3×10 mL). The combined organic layers were washed with brine (1×30 mL), dried over anhydrous Na₂SO₄. After filtration, the filtrate was concentrated under reduced pressure. The residue was purified by reverse flash chromatography (Condition 5, Gradient 2) to afford tert-butyl (R)-(1-(2-ethyl-7-((8-fluoro-2-methylimidazo[1,2-a]pyridin-6-yl)carbamoyl)-2H-indazol-4-yl)pyrrolidin-3-yl)(methyl)carbamate (160 mg, 69%) as a solid. LCMS (ES, m/z): 536 [M+H]⁺

### Synthesis of Compound 393

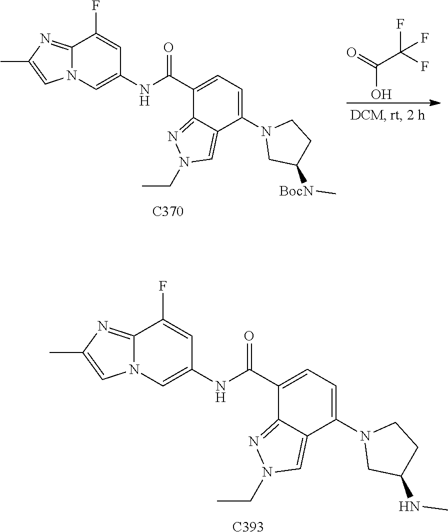

A solution of tert-butyl tert-butyl (R)-(1-(2-ethyl-7-((8-fluoro-2-methylimidazo[1,2-a]pyridin-6-yl)carbamoyl)-2H-indazol-4-yl)pyrrolidin-3-yl)(methyl)carbamate (135 mg, 0.25 mmol, 1 equiv) in trifluoroacetic acid (2 mL) and DCM (2 mL) was stirred for 2 h at room temperature. The resulting mixture was concentrated under reduced pressure. The residue was basified to pH 8 with 7 M NH₃(g) in MeOH. The resulting mixture was concentrated under reduced pressure. The residue was purified by prep-HPLC (Condition 12, Gradient 3) to afford (R)-2-ethyl-N-(8-fluoro-2-methylimidazo[1,2-a]pyridin-6-yl)-4-(3-(methylamino)pyrrolidin-1-yl)-2H-indazole-7-carboxamide (12 mg, 11%) as a solid. LCMS (ES, m/z): 436 [M+H]⁺1H NMR (300 MHz, DMSO-d6) δ 11.05 (s, 1H), 9.19 (d, J=1.6 Hz, 1H), 8.84 (s, 1H), 7.97-7.85 (m, 2H), 7.26 (dd, J=12.4, 1.6 Hz, 1H), 6.01 (d, J=8.4 Hz, 1H), 4.57 (q, J=7.3 Hz, 2H), 3.76 (dq, J=13.7, 7.1, 6.3 Hz, 1H), 3.65 (d, J=7.4 Hz, 3H), 3.42 (dd, J=10.2, 4.0 Hz, 2H), 2.35 (s, 6H), 2.14 (dd, J=11.2, 4.5 Hz, 1H), 1.92 (dd, J=11.8, 6.1 Hz, 1H), 1.61 (t, J=7.2 Hz, 3H).

### Example 231: Synthesis of Compound 423

### Synthesis of Intermediate C371

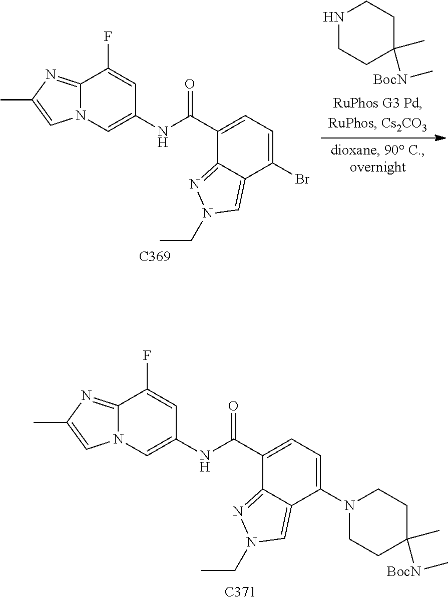

To a stirred solution of 4-bromo-2-ethyl-N-{8-fluoro-2-methylimidazo[1,2-a]pyridin-6-yl}indazole-7-carboxamide (180 mg, 0.432 mmol, 1 equiv) and tert-butyl N-methyl-N-(4-methylpiperidin-4-yl)carbamate (99 mg, 0.432 mmol, 1 equiv) in dioxane (5 mL) were added Cs₂CO₃ (423 mg, 1.296 mmol, 3 equiv), RuPhos (40 mg, 0.086 mmol, 0.2 equiv) and RuPhos Palladacycle Gen.3 (36 mg, 0.043 mmol, 0.1 equiv) in portions at room temperature under nitrogen atmosphere. The resulting mixture was stirred for overnight at 90° C. under nitrogen atmosphere. The mixture was allowed to cool down to room temperature. The resulting mixture was concentrated under reduced pressure. The residue was purified by silica gel column chromatography, eluted with PE/EA (1:10) to afford tert-butyl N-{1-[2-ethyl-7-({8-fluoro-2-methylimidazo[1,2-a]pyridin-6-yl}carbamoyl) indazol-4-yl]-4-methylpiperidin-4-yl}-N-methylcarbamate (210 mg, 86%) as a solid. LCMS (ES, m/z): 564 [M+H]⁺

### Synthesis of Compound 423

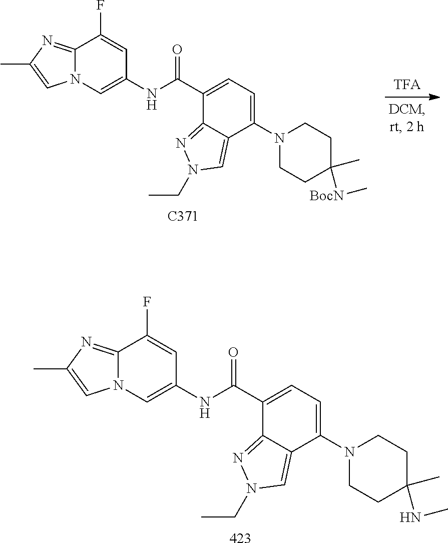

A solution of tert-butyl N-{1-[2-ethyl-7-({8-fluoro-2-methylimidazo[1,2-a]pyridin-6-yl}carbamoyl) indazol-4-yl]-4-methylpiperidin-4-yl}-N-methylcarbamate (200 mg, 0.355 mmol, 1 equiv) in TFA (2 mL) and DCM (2 mL) was stirred for 2 h at room temperature. The resulting mixture was concentrated under reduced pressure. The residue was basified to pH 8 with 7 M NH₃(g) in MeOH. The resulting mixture was concentrated under vacuum. The residue was purified by reverse flash chromatography (Condition 3, Gradient 8) to afford 2-ethyl-N-{8-fluoro-2-methylimidazo[1,2-a]pyridin-6-yl}-4-[4-methyl-4-(methylamino)piperidin-1-yl]indazole-7-carboxamide (50 mg, 30%) as a solid. LCMS (ES, m/z): 464 [M+H]^{+ 1}H NMR (400 MHz, DMSO-d₆) δ 11.10 (s, 1H), 9.20 (d, J=1.6 Hz, 1H), 8.80 (s, 1H), 7.96 (d, J=8.1 Hz, 1H), 7.90 (dd, J=3.2, 1.0 Hz, 1H), 7.29 (dd, J=12.3, 1.7 Hz, 1H), 6.47 (d, J=8.2 Hz, 1H), 4.59 (q, J=7.3 Hz, 2H), 3.55-3.43 (m, 4H), 2.38-2.33 (m, 3H), 2.23 (s, 3H), 1.70 (dt, J=13.3, 4.6 Hz, 2H), 1.61 (q, J=7.4 Hz, 5H), 1.09 (s, 3H).

### Example 232: Synthesis of Compound 452

### Synthesis of Intermediate C373

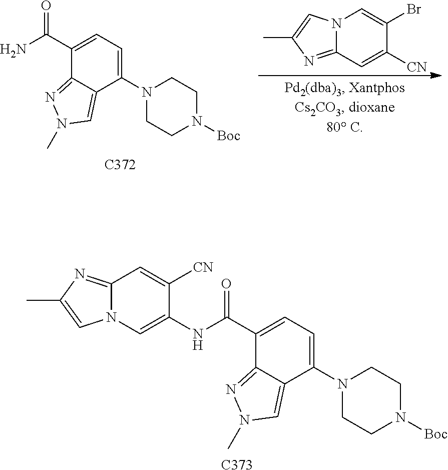

To a solution of tert-butyl 4-(7-carbamoyl-2-methylindazol-4-yl) piperazine-1-carboxylate (300 mg, 0.835 mmol, 1 equiv) and 6-bromo-2-methylimidazo[1,2-a]pyridine-7-carbonitrile (197.04 mg, 0.835 mmol, 1 equiv) in dioxane (5 mL) were added Cs₂CO₃ (815.84 mg, 2.505 mmol, 3 equiv), Pd₂(dba)₃ (76.43 mg, 0.084 mmol, 0.1 equiv) and XantPhos (48.30 mg, 0.084 mmol, 0.1 equiv). After stirring for 2 h at 100° C. under a nitrogen atmosphere. The resulting mixture was concentrated under reduced pressure. The residue was purified by silica gel column chromatography, eluted with PE:EA (1:1-1:10) to afford tert-butyl 4-[7-({7-cyano-2-methylimidazo[1,2-a]pyridin-6-yl}carbamoyl)-2-methylindazol-4-yl]piperazine-1-carboxylate (400 mg, 93.13%) as a solid. ¹H NMR (400 MHz, DMSO-d₆) δ 11.47 (s, 1H), 9.66 (s, 1H), 8.85 (s, 1H), 8.29 (s, 1H), 8.06-7.96 (m, 2H), 6.52 (d, J=8.2 Hz, 1H), 4.26 (s, 3H), 3.57 (d, J=5.7 Hz, 5H), 3.48 (d, J=5.7 Hz, 3H), 2.40 (s, 3H), 1.45 (s, 9H).

### Synthesis of Compound 452

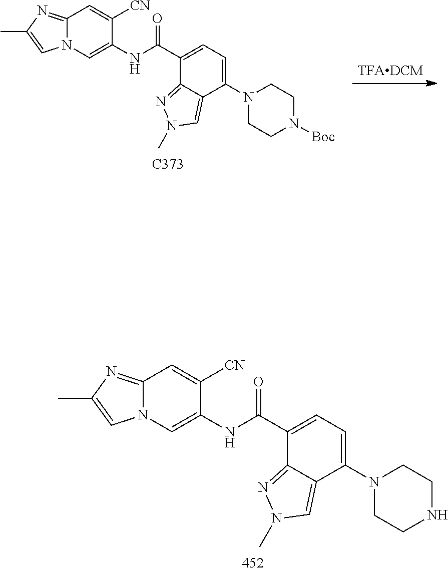

A solution of tert-butyl 4-[7-({7-cyano-2-methylimidazo[1,2-a]pyridin-6-yl}carbamoyl)-2-methylindazol-4-yl]piperazine-1-carboxylate (80 mg, 0.155 mmol, 1 equiv) in DCM (4 mL) was added TFA (1 mL) dropwise at room temperature. The resulting mixture was stirred for 2 h at room temperature. The resulting mixture was concentrated under reduced pressure. The residue was purified by reverse flash chromatography (Condition 5, Gradient 4) to afford N-{7-cyano-2-methylimidazo[1,2-a]pyridin-6-yl}-2-methyl-4-(piperazin-1-yl) indazole-7-carboxamide (4 mg, 6%) as a solid. LCMS (ES, m/z): 515 [M+H]^{+ 1}H NMR (400 MHz, Methanol-d₄) δ 11.48 (s, 1H), 9.67 (s, 1H), 8.82 (s, 1H), 8.29 (s, 1H), 8.08-7.97 (m, 2H), 6.51 (d, J=8.2 Hz, 1H), 4.25 (s, 3H), 3.39 (t, J=5.0 Hz, 4H), 2.91 (t, J=5.0 Hz, 4H), 2.40 (s, 3H).

### Example 233: Synthesis of Compound 475

### Synthesis of Intermediate C375

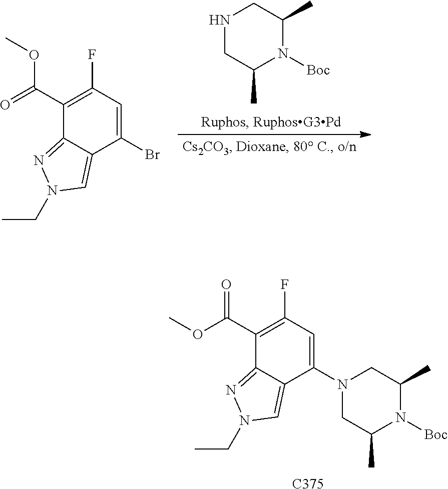 [SMILES: CCn1cc2c(Br)cc(F)c(C(=O)OC)c2n1 ; C[C@@H]1CNC[C@H](C)N1C(=O)OC(C)(C)C ; CCn1cc2c(N3C[C@@H](C)N(C(=O)OC(C)(C)C)[C@@H](C)C3)cc(F)c(C(=O)OC)c2n1 ; "C375"]

To a stirred mixture of methyl 4-bromo-2-ethyl-6-fluoroindazole-7-carboxylate (110 mg, 0.365 mmol, 1 equiv) and tert-butyl (2R,6S)-2,6-dimethylpiperazine-1-carboxylate (93.95 mg, 0.438 mmol, 1.2 equiv) in dioxane (2 mL) was added Cs₂CO₃ (357.07 mg, 1.095 mmol, 3 equiv), Ruphos (34.09 mg, 0.073 mmol, 0.2 equiv) and RuPhos Palladacycle Gen.3 (30.55 mg, 0.036 mmol, 0.1 equiv) at room temperature under nitrogen atmosphere. The resulting mixture was stirred overnight at 80° C. under nitrogen atmosphere. The resulting mixture was concentrated under reduced pressure. The residue was purified by silica gel column chromatography, eluted with PE/EA (1:1) to afford methyl 4-[(3R,5S)-4-(tert-butoxycarbonyl)-3,5-dimethylpiperazin-1-yl]-2-ethyl-6-fluoroindazole-7-carboxylate (95 mg, 60%) as a solid. LCMS (ES, m/z): 435 [M+H]⁺

### Synthesis of Intermediate C376

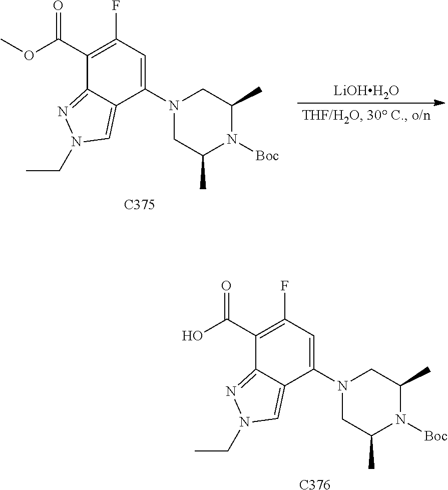 [SMILES: CCn1cc2c(N3C[C@@H](C)N(C(=O)OC(C)(C)C)[C@@H](C)C3)cc(F)c(C(=O)OC)c2n1 ; "LiOH·H2O" ; "C375" ; CCn1cc2c(N3C[C@@H](C)N(C(=O)OC(C)(C)C)[C@@H](C)C3)cc(F)c(C(=O)O)c2n1 ; "C376"]

To a stirred solution of methyl 4-[(3R,5S)-4-(tert-butoxycarbonyl)-3,5-dimethylpiperazin-1-yl]-2-ethyl-6-fluoroindazole-7-carboxylate (95 mg, 0.219 mmol, 1 equiv) in THF (1.2 mL) and H₂O (0.4 mL) was added lithiumol hydrate (18.35 mg, 0.438 mmol, 2 equiv) at room temperature. The resulting mixture was stirred overnight at 30° C. The resulting mixture was concentrated under vacuum. The resulting mixture was diluted with H₂O (5 mL). The mixture was acidified to pH 4 with citric acid and extracted with DCM (3×10 mL). The combined organic layers were washed with brine (1×10 mL), dried over anhydrous Na₂SO₄. After filtration, the filtrate was concentrated under reduced pressure to afford 4-[(3R,5S)-4-(tert-butoxycarbonyl)-3,5-dimethylpiperazin-1-yl]-2-ethyl-6-fluoroindazole-7-carboxylic acid (85 mg, 92%) as a solid. LCMS (ES, m/z): 419 [M−H]⁻

### Synthesis of Intermediate C377

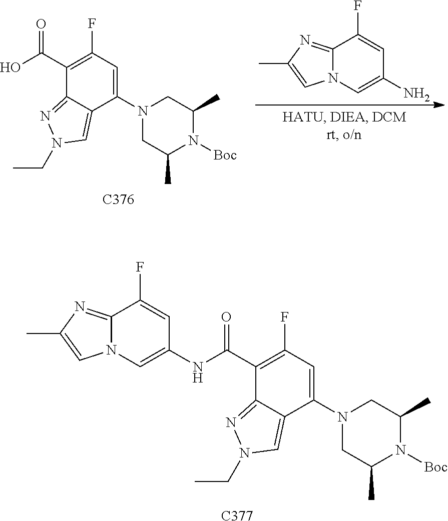

To a stirred mixture of 4-[(3R,5S)-4-(tert-butoxycarbonyl)-3,5-dimethylpiperazin-1-yl]-2-ethyl-6-fluoroindazole-7-carboxylic acid (85 mg, 0.202 mmol, 1 equiv) and 8-fluoro-2-methylimidazo[1,2-a]pyridin-6-amine hydrochloride (44.83 mg, 0.222 mmol, 1.10 equiv) in DCM (2 mL) was added DIEA (130.63 mg, 1.010 mmol, 5 equiv) and HATU (99.92 mg, 0.263 mmol, 1.3 equiv) at room temperature. The resulting mixture was stirred overnight at room temperature. The resulting mixture was concentrated under reduced pressure. The residue was purified by silica gel column chromatography, eluted with PE/EA (2:1) to afford tert-butyl (2R,6S)-4-[2-ethyl-6-fluoro-7-({8-fluoro-2-methylimidazo[1,2-a]pyridin-6-yl}carbamoyl) indazol-4-yl]-2,6-dimethylpiperazine-1-carboxylate (80 mg, 70%) as a solid. LCMS (ES, m/z): 568 [M−H]⁻

### Synthesis of Compound 475

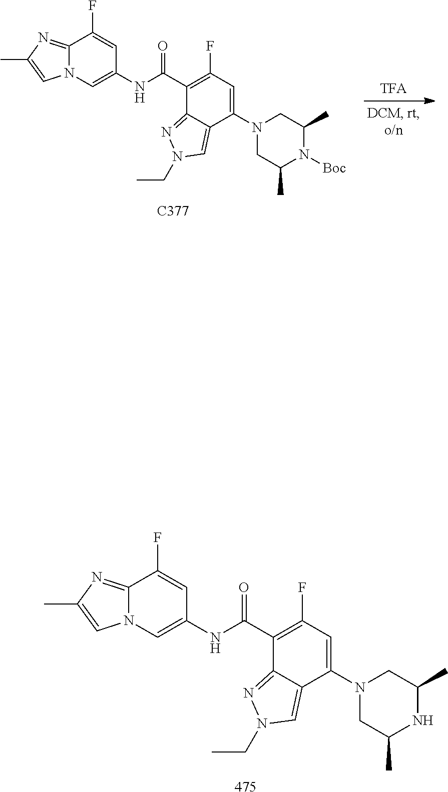

To a stirred solution of tert-butyl (2R,6S)-4-[2-ethyl-6-fluoro-7-({8-fluoro-2-methylimidazo[1,2-a]pyridin-6-yl}carbamoyl) indazol-4-yl]-2,6-dimethylpiperazine-1-carboxylate (80 mg, 0.141 mmol, 1 equiv) in DCM (1 mL) was added TFA (1 mL) at room temperature. The resulting mixture was stirred for 2 h at room temperature. The resulting mixture was concentrated under reduced pressure. The crude product was purified by Prep-HPLC (Condition 15, Gradient 3) to afford 4-[(3R,5S)-3,5-dimethylpiperazin-1-yl]-2-ethyl-6-fluoro-N-{8-fluoro-2-methylimidazo[1,2-a]pyridin-6-yl}indazole-7-carboxamide; trifluoroacetic acid (31.4 mg, 38%) as a solid. LCMS (ES, m/z): 468 [M+H]^{+ 1}H NMR (400 MHz, DMSO-d₆) δ 11.07 (s, 1H), 9.40 (s, 1H), 9.19 (s, 1H), 8.91 (s, 1H), 8.56 (d, J=11.5 Hz, 1H), 8.11 (s, 1H), 7.58 (d, J=12.2 Hz, 1H), 6.48 (d, J=14.4 Hz, 1H), 4.52 (q, J=7.3 Hz, 2H), 4.04 (d, J=13.2 Hz, 2H), 2.95 (t, J=12.4 Hz, 2H), 2.44 (d, J=4.9 Hz, 3H), 1.58 (t, J=7.3 Hz, 3H), 1.32 (d, J=6.4 Hz, 6H).

### Example 234: Synthesis of Compound 497

### Synthesis of Intermediate C378

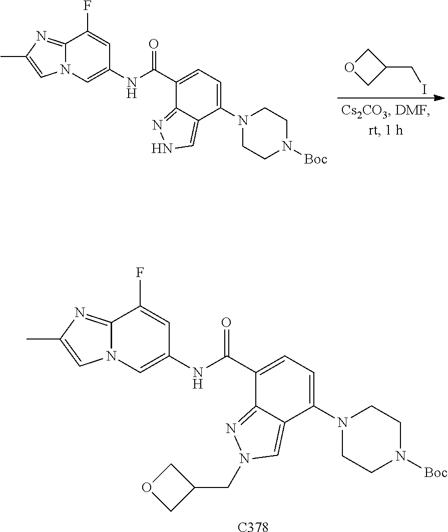 [SMILES: Cc1cn2cc(NC(=O)c3ccc(N4CCN(C(=O)OC(C)(C)C)CC4)c4c[nH]nc34)cc(F)c2n1 ; ICC1COC1 ; Cc1cn2cc(NC(=O)c3ccc(N4CCN(C(=O)OC(C)(C)C)CC4)c4cn(CC5COC5)nc34)cc(F)c2n1 ; "C378"]

To a stirred solution of tert-butyl 4-[7-({8-fluoro-2-methylimidazo[1,2-a]pyridin-6-yl}carbamoyl)-2H-indazol-4-yl]piperazine-1-carboxylate (110 mg, 0.223 mmol, 1.0 equiv) and 3-(iodomethyl)oxetane (66.20 mg, 0.335 mmol, 1.5 equiv) in DMF (2.2 mL) was added Cs₂CO₃ (217.8 mg, 0.669 mmol, 3.0 equiv) at room temperature. The resulting mixture was stirred for 1 h at room temperature. The resulting mixture was diluted with water (10 mL) and extracted with EtOAc (3×10 mL). The combined organic layer was washed with water (3×10 mL), dried over anhydrous Na₂SO₄. After filtration, the filtrate was concentrated under reduced pressure. The residue was purified by silica gel column chromatography, eluted with EA (100%) to afford tert-butyl 4-[7-({8-fluoro-2-methylimidazo[1,2-a]pyridin-6-yl}carbamoyl)-2-(oxetan-3-ylmethyl) indazol-4-yl]piperazine-1-carboxylate (65 mg, 52%) as a solid. LCMS (ES, m/z): 423.2 [M+H]⁺

### Synthesis of Compound 497

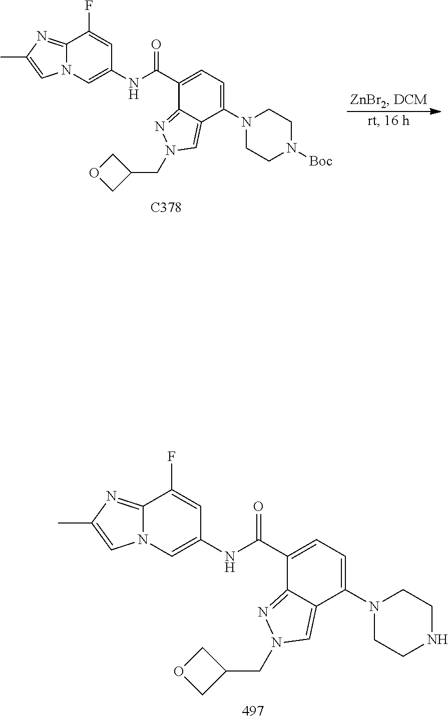

To a stirred solution of tert-butyl 4-[7-({8-fluoro-2-methylimidazo[1,2-a]pyridin-6-yl}carbamoyl)-2-(oxetan-3-ylmethyl) indazol-4-yl]piperazine-1-carboxylate (20 mg, 0.035 mmol, 1.0 equiv) in DCM (0.5 mL) was added ZnBr₂ (79.91 mg, 0.350 mmol, 10 equiv) at room temperature. The resulting mixture was stirred for 16 h at room temperature. The resulting mixture was diluted with water (2 mL) and extracted with CH₂Cl₂ (2×2 mL), dried over anhydrous Na₂SO₄. After filtration, the filtrate was concentrated under reduced pressure. The crude product was purified by Prep-HPLC (Condition 10, Gradient 2) to afford N-{8-fluoro-2-methylimidazo[1,2-a]pyridin-6-yl}-2-(oxetan-3-ylmethyl)-4-(piperazin-1-yl) indazole-7-carboxamide (5.6 mg, 34%) as a solid. LCMS (ES, m/z): 403.2 [M+H]^{+ 1}H NMR (400 MHz, Methanol-d₄) δ 9.02 (s, 1H), 8.55 (s, 1H), 8.06 (d, J=8.0 Hz, 1H), 7.69-7.63 (m, 1H), 7.14 (d, J=11.8 Hz, 1H), 6.50 (d, J=8.2 Hz, 1H), 4.95-4.82 (m, 4H), 4.71 (t, J=6.1 Hz, 2H), 3.82-3.72 (m, 1H), 3.42 (t, J=4.9 Hz, 4H), 3.07 (m, 4H), 2.42 (s, 3H)).

### Example 235: Synthesis of Compound 498

### Synthesis of Intermediate C379

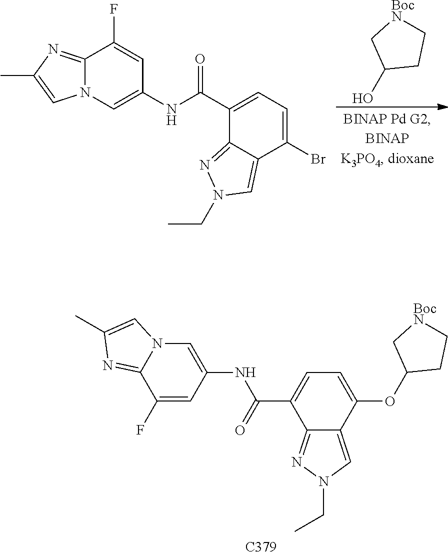

To a solution of 4-bromo-2-ethyl-N-{8-fluoro-2-methylimidazo[1,2-a]pyridin-6-yl}indazole-7-carboxamide (200 mg, 0.480 mmol, 1 equiv) and tert-butyl 3-hydroxypyrrolidine-1-carboxylate (269.8 mg, 1.440 mmol, 3.0 equiv) in dioxane (5 mL) were added K₃PO₄ (305.9 mg, 1.440 mmol, 3.0 equiv), BINAP (29.9 mg, 0.048 mmol, 0.1 equiv) and Binap Palladacycle Gen. 2 (44.8 mg, 0.048 mmol, 0.1 equiv). After stirring for 12 h at 100° C. under a nitrogen atmosphere, the resulting mixture was concentrated under reduced pressure. The residue was purified by reverse flash chromatography (Condition 3, Gradient 1) to afford tert-butyl 3-{[2-ethyl-7-({8-fluoro-2-methylimidazo[1,2-a]pyridin-6-yl}carbamoyl) indazol-4-yl]oxy}pyrrolidine-1-carboxylate (80 mg, 32%) as a solid. LCMS (ES, m/z): 523 [M+H]⁺

### Synthesis of Compound 498

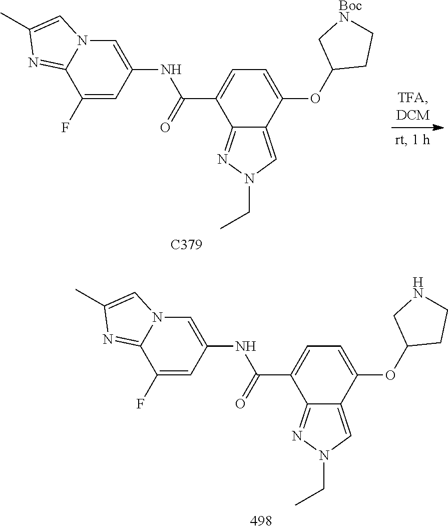

A solution of tert-butyl 3-{[2-ethyl-7-({8-fluoro-2-methylimidazo[1,2-a]pyridin-6-yl}carbamoyl) indazol-4-yl]oxy}pyrrolidine-1-carboxylate (80 mg, 0.153 mmol, 1 equiv) and TFA (0.2 mL, 2.693 mmol) in DCM (2 mL) was stirred for 1 h at room temperature. The resulting mixture was concentrated under reduced pressure. The residue was purified by reverse flash chromatography (Condition 6, Gradient 1) to afford 2-ethyl-N-{8-fluoro-2-methylimidazo[1,2-a]pyridin-6-yl}-4-(pyrrolidin-3-yloxy) indazole-7-carboxamide (30 mg, 46%) as a solid. LCMS (ES, m/z): 423[M+H]^{+ 1}H NMR (300 MHz, DMSO-d₆) δ 11.07 (s, 1H), 9.41 (d, J=1.5 Hz, 1H), 9.29 (s, 1H), 9.13 (s, 1H), 8.68 (s, 1H), 8.10 (d, J=8.1 Hz, 2H), 7.71 (d, J=12.0 Hz, 1H), 6.75 (d, J=8.2 Hz, 1H), 5.43 (s, 1H), 4.64 (q, J=7.3 Hz, 2H), 3.65-3.35 (m, 4H), 3.51 (s, 1H), 2.43 (d, J=0.9 Hz, 3H), 2.41-2.20 (m, 2H), 1.62 (t, J=7.3 Hz, 3H).

### Example 236: Synthesis of Compound 499

### Synthesis of Intermediate C380

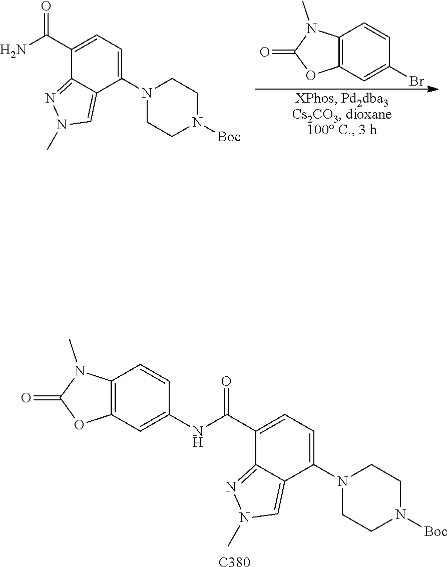

To a solution of 6-bromo-3-methylbenzo[d]oxazol-2(3H)-one (150 mg, 0.658 mmol, 1 equiv) and tert-butyl 4-(7-carbamoyl-2-methylindazol-4-yl)piperazine-1-carboxylate (263 mg, 0.658 mmol, 1 equiv) in dioxane (8 mL) were added Cs₂CO₃ (643 mg, 1.974 mmol, 3.0 equiv), XantPhos (76 mg, 0.131 mmol, 0.2 equiv) and Pd₂(dba)₃ (60 mg, 0.0658 mmol, 0.1 equiv). After stirring for 3 h at 90° C. under nitrogen atmosphere, the resulting mixture was concentrated under reduced pressure. The residue was purified by silica gel column chromatography, eluted with PE:EA (0:100) to tert-butyl 4-(2-methyl-7-((3-methyl-2-oxo-2,3-dihydrobenzo[d]oxazol-6-yl)carbamoyl)-2H-indazol-4-yl)piperazine-1-carboxylate (150 mg, 43%) as a solid. LCMS (ES, m/z): 507 [M+H]⁺

### Synthesis of Compound 499

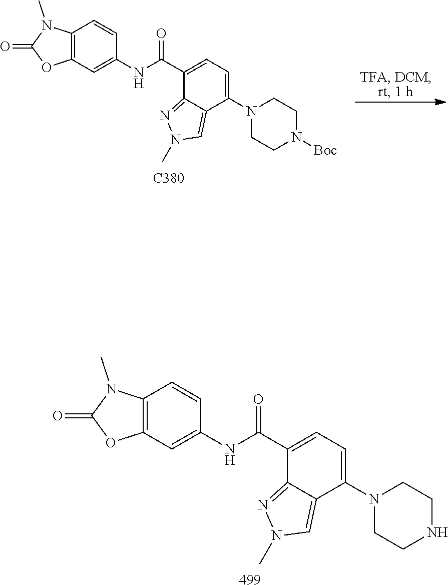

A solution of tert-butyl 4-(2-methyl-7-((3-methyl-2-oxo-2,3-dihydrobenzo[d]oxazol-6-yl)carbamoyl)-2H-indazol-4-yl)piperazine-1-carboxylate (150 mg, 0.296 mmol, 1 equiv) and TFA (0.5 mL) in DCM (4 mL) was stirred for 1 h at room temperature. The mixture was basified to pH 8 with NH₃(g) in MeOH. The resulting mixture was concentrated under reduced pressure. The residue was purified by reverse flash chromatography (Condition 3, Gradient 2) to afford 2-methyl-N-(3-methyl-2-oxo-2,3-dihydrobenzo[d]oxazol-6-yl)-4-(piperazin-1-yl)-2H-indazole-7-carboxamide (80 mg, 67%) as a solid. LCMS (ES, m/z): 407 [M+H]^{+ 1}H NMR (300 MHz, DMSO-d₆) δ 11.23 (s, 1H), 8.78 (s, 1H), 8.03 (d, J=1.9 Hz, 1H), 7.98 (d, J=8.0 Hz, 1H), 7.47 (dd, J=8.4, 2.0 Hz, 1H), 7.25 (d, J=8.4 Hz, 1H), 6.47 (d, J=8.1 Hz, 1H), 4.28 (s, 3H), 3.35 (s, 4H), 3.32 (s, 3H), 2.91 (t, J=4.8 Hz, 4H).

### Example 237: Synthesis of Compound 500

### Synthesis of Intermediate C381

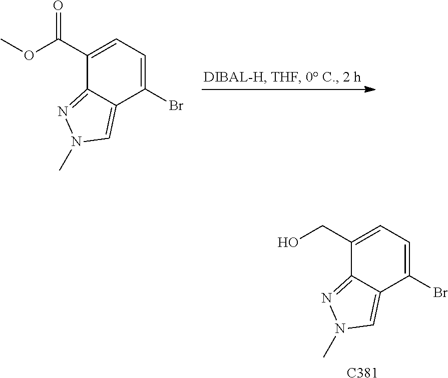

A solution of methyl 4-bromo-2-methylindazole-7-carboxylate (550 mg, 2.044 mmol, 1.0 equiv) in THF (6 mL) was treated with DIBAL-H (6.13 mL, 6.132 mmol, 3.0 equiv) at 0° C. under nitrogen atmosphere. The resulting mixture was stirred for 2 h at 0° C. under nitrogen atmosphere. The reaction was quenched with water at 0° C. The resulting mixture was extracted with EtOAc (3×20 mL). The combined organic layers were washed with brine (1×10 mL), dried over anhydrous Na₂SO₄. After filtration, the filtrate was concentrated under reduced pressure to afford (4-bromo-2-methylindazol-7-yl)methanol (505 mg, 100%) as a solid. LCMS (ES, m/z): 241 [M+H]⁺

### Synthesis of Intermediate C382

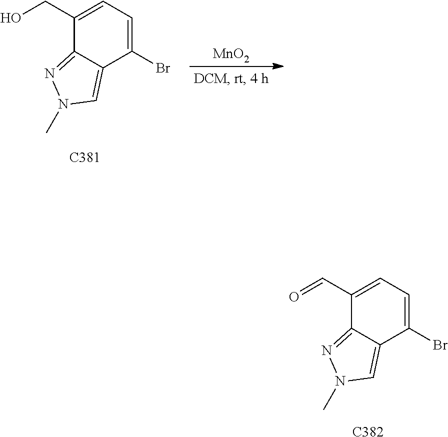

A solution of (4-bromo-2-methylindazol-7-yl)methanol (505 mg, 2.095 mmol, 1 equiv) in DCM (5 mL) was treated with manganese dioxide (1821.0 mg, 20.950 mmol, 10 equiv) at room temperature under nitrogen atmosphere. The resulting mixture was stirred for 4 h at room temperature under nitrogen atmosphere. The resulting mixture was filtered and the filter cake was washed with DCM (2×5 mL). The filtrate was concentrated under reduced pressure to afford 4-bromo-2-methylindazole-7-carbaldehyde (440 mg, 88%) as a solid. LCMS (ES, m/z): 239 [M+H]⁺

### Synthesis of Intermediate C383

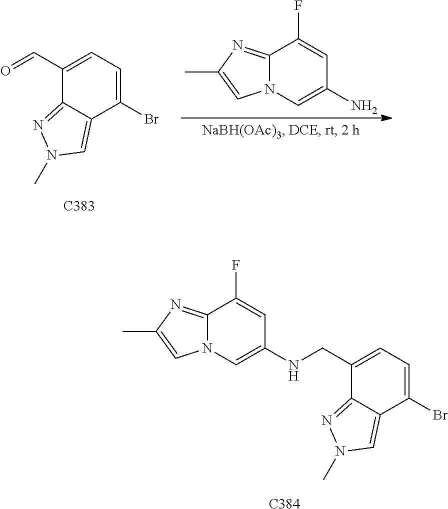

To a stirred mixture of 4-bromo-2-methylindazole-7-carbaldehyde (440 mg, 1.840 mmol, 1.0 equiv) and 8-fluoro-2-methylimidazo[1,2-a]pyridin-6-amine (364.8 mg, 2.208 mmol, 1.2 equiv) in DCM (5 mL) was added NaBH(OAc)₃ (780.1 mg, 3.680 mmol, 2.0 equiv) in portions at room temperature under nitrogen atmosphere. The resulting mixture was stirred for 2 h at room temperature under nitrogen atmosphere. The reaction was quenched with Water at room temperature. The resulting mixture was extracted with EtOAc (3×20 mL). The combined organic layers were washed with brine (1×10 mL), dried over anhydrous Na₂SO₄. After filtration, the filtrate was concentrated under reduced pressure. The residue was purified by silica gel column chromatography, eluted with EA to afford N-[(4-bromo-2-methylindazol-7-yl)methyl]-8-fluoro-2-methylimidazo[1,2-a]pyridin-6-amine (610 mg, 85%) as a solid. LCMS (ES, m/z): 388 [M+H]⁺

### Synthesis of Intermediate C385

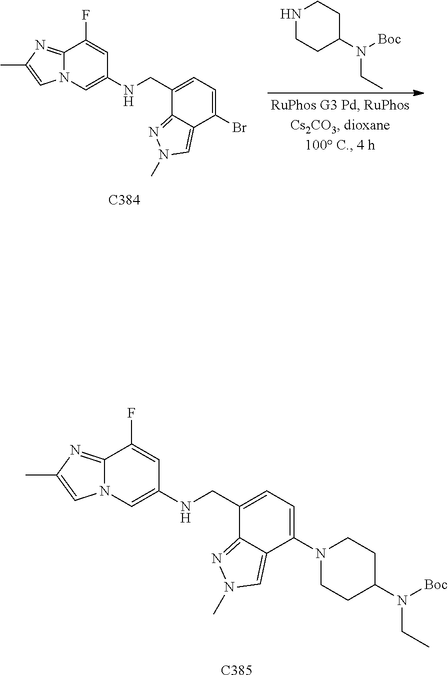 [SMILES: Cc1cn2cc(NCc3ccc(Br)c4cn(C)nc34)cc(F)c2n1 ; CCN(C(=O)OC(C)(C)C)C1CCNCC1 ; "C384" ; CCN(C(=O)OC(C)(C)C)C1CCN(c2ccc(CNc3cc(F)c4nc(C)cn4c3)c3nn(C)cc23)CC1 ; "C385"]

To a stirred mixture of N-[(4-bromo-2-methylindazol-7-yl)methyl]-8-fluoro-2-methylimidazo[1,2-a]pyridin-6-amine (300 mg, 0.773 mmol, 1.0 equiv) and tert-butyl N-ethyl-N-(piperidin-4-yl)carbamate (264.7 mg, 1.159 mmol, 1.5 equiv) in dioxane (3 mL) were added Cs₂CO₃ (755.3 mg, 2.319 mmol, 3.0 equiv), RuPhos (72.1 mg, 0.155 mmol, 0.2 equiv) and RuPhos Palladacycle Gen.3 (64.6 mg, 0.077 mmol, 0.1 equiv) at room temperature under nitrogen atmosphere. The resulting mixture was stirred for 4 h at 100° C. under nitrogen atmosphere. The residue was purified by silica gel column chromatography, eluted with CH₂Cl₂/MeOH (10:1) to afford tert-butyl N-ethyl-N-(1-{7-[({8-fluoro-2-methylimidazo[1,2-a]pyridin-6-yl}amino)methyl]-2-methylindazol-4-yl}piperidin-4-yl)carbamate (180 mg, 43%) as a solid. LCMS (ES, m/z): 536 [M+H]⁺

### Synthesis of Compound 500

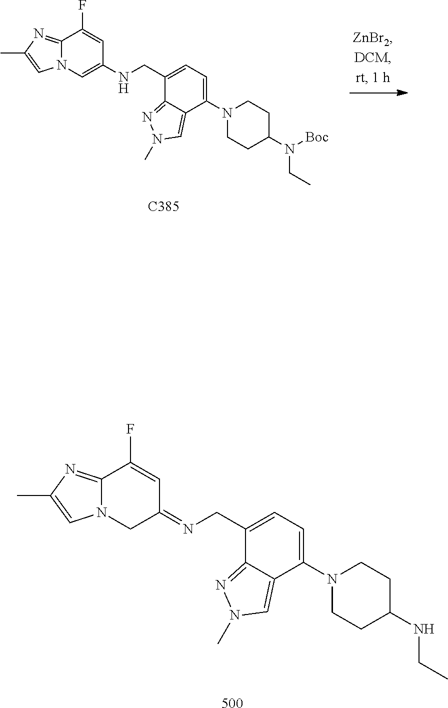 [SMILES: CCN(C(=O)OC(C)(C)C)C1CCN(c2ccc(CNc3cc(F)c4nc(C)cn4c3)c3nn(C)cc23)CC1 ; "C385" ; CCNC1CCN(c2ccc(C/N=C3\C=C(F)c4nc(C)cn4C3)c3nn(C)cc23)CC1 ; "500"]

A solution of tert-butyl N-ethyl-N-(1-{7-[({8-fluoro-2-methylimidazo[1,2-a]pyridin-6-yl}amino)methyl]-2-methylindazol-4-yl}piperidin-4-yl)carbamate (160 mg, 0.299 mmol, 1.0 equiv) in DCM (2 mL) was treated with ZnBr₂ (336.3 mg, 1.495 mmol, 5.0 equiv) at room temperature. The resulting mixture was stirred for 1 h at room temperature. The crude product was purified by Prep-HPLC (Condition 10, Gradient 3) to afford N-ethyl-1-[7-({[(6E)-8-fluoro-2-methyl-5H-imidazo[1,2-a]pyridin-6-ylidene]amino}methyl)-2-methylindazol-4-yl]piperidin-4-amine (8 mg, 6.15%) as a brown solid. LCMS (ES, m/z): 436 [M+H]^{+ 1}H NMR (400 MHz, DMSO-d₆) δ 8.40 (s, 1H), 7.41 (d, J=3.2 Hz, 1H), 6.84 (d, J=12.9 Hz, 1H), 6.71 (d, J=7.4 Hz, 1H), 6.17 (d, J=7.5 Hz, 1H), 5.18 (s, 2H), 4.33 (s, 2H), 4.20 (s, 3H), 3.54 (d, J=12.0 Hz, 2H), 2.68 (t, J=11.7 Hz, 2H), 2.58 (d, J=7.1 Hz, 3H), 2.21 (s, 3H), 1.91 (d, J=12.3 Hz, 2H), 1.42 (q, J=11.0 Hz, 2H), 1.02 (t, J=7.1 Hz, 3H).

### Example 238: Synthesis of Compound 501

### Synthesis of Intermediate C387

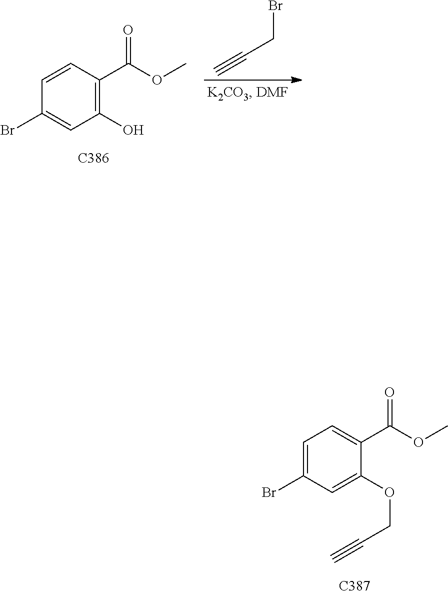

To a stirred solution of methyl 4-bromo-2-hydroxybenzoate (10 g, 43.282 mmol, 1 equiv) and K₂CO₃ (17.95 g, 129.846 mmol, 3.0 equiv) in DMF (150 mL) was added propargyl bromide (7.72 g, 64.923 mmol, 1.5 equiv) dropwise at room temperature. The resulting mixture was stirred for 16 h at 50° C. The mixture was allowed to cool down to room temperature. The resulting mixture was diluted with water (500 mL). The resulting mixture was extracted with EA (3×100 mL). The combined organic layers were washed with brine (2×50 mL), dried over anhydrous Na₂SO₄. After filtration, the filtrate was concentrated under reduced pressure to afford methyl 4-bromo-2-(prop-2-yn-1-yloxy)benzoate (10.1 g, 87%) as a solid. ¹H NMR (400 MHz, DMSO-d₆) δ 7.62 (d, J=8.2 Hz, 1H), 7.45 (d, J=1.8 Hz, 1H), 7.29 (dd, J=8.3, 1.8 Hz, 1H), 4.96 (d, J=2.4 Hz, 2H), 3.80 (s, 3H), 3.65 (t, J=2.4 Hz, 1H).

### Synthesis of Intermediate C388

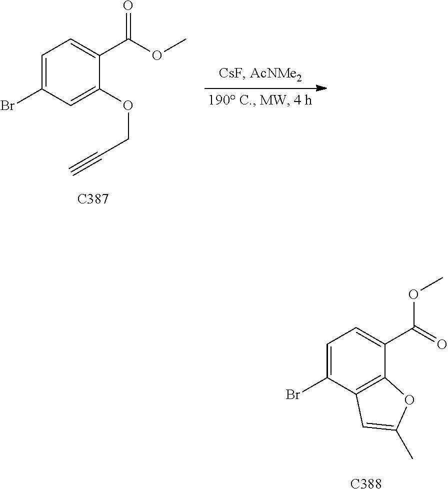

A mixture of methyl 4-bromo-2-(prop-2-yn-1-yloxy)benzoate (5.0 g, 18.581 mmol, 1 equiv) and CsF (2.82 g, 18.581 mmol, 1.0 equiv) in DMA (50 mL) was irradiated with microwave for 4 h at 190° C. The reaction was quenched with water (200 mL). The resulting mixture was extracted with EA (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (3:1) to afford methyl 4-bromo-2-methyl-1-benzofuran-7-carboxylate (2.0 g, 40%) as a solid. LCMS (ES, m/z): 269 [M+H]⁺

### Synthesis of Intermediate C389

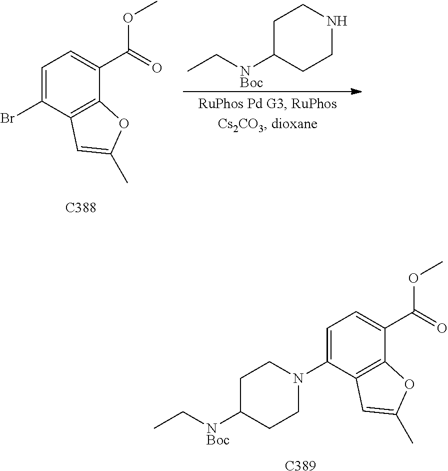

To a solution of methyl 4-bromo-2-methyl-1-benzofuran-7-carboxylate (670 mg, 2.490 mmol, 1 equiv) and tert-butyl N-ethyl-N-(piperidin-4-yl)carbamate (852.78 mg, 3.735 mmol, 1.5 equiv) in dioxane (20 mL) were added Cs₂CO₃ (1622.47 mg, 4.980 mmol, 2.0 equiv), RuPhos (116.03 mg, 0249 mmol, 0.1 equiv) and 3rd Generation RuPhos precatalyst (208.24 mg, 0.249 mmol, 0.1 equiv). After stirring for 4 h at 90° C. under nitrogen atmosphere, the resulting mixture was concentrated under reduced pressure. The residue was purified by silica gel column chromatography, eluted with PE/EA (1:10) to afford methyl 4-{4-[(tert-butoxycarbonyl)(ethyl)amino]piperidin-1-yl}-2-methyl-1-benzofuran-7-carboxylate (750 mg, 72%) as a solid. LCMS (ES, m/z): 417 [M+H]⁺

### Synthesis of Intermediate C390

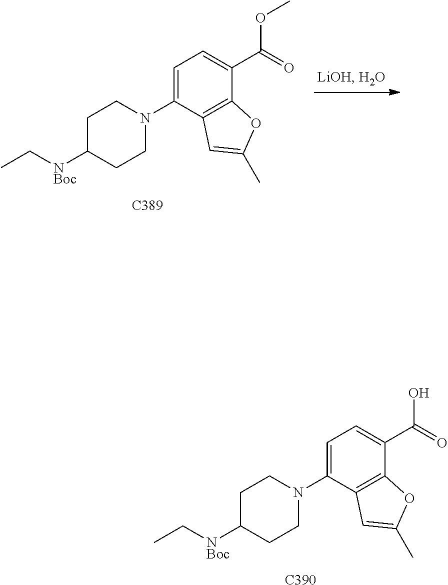

To a stirred solution of methyl 4-{4-[(tert-butoxycarbonyl)(ethyl)amino]piperidin-1-yl}-2-methyl-1-benzofuran-7-carboxylate (720 mg, 1.729 mmol, 1 equiv) in H₂O (5 mL), MeOH (10 mL) and THF (10 mL) were added LiOH (248.40 mg, 10.374 mmol, 6.0 equiv) in portions at room temperature. The resulting mixture was stirred for 2 h at 50° C. The resulting mixture was concentrated under reduced pressure. The residue was acidified to pH 6 with 1 N HCl. The precipitated solids were collected by filtration and washed with water (2×20 mL). The resulting solids were dried to afford 4-{4-[(tert-butoxycarbonyl)(ethyl)amino]piperidin-1-yl}-2-methyl-1-benzofuran-7-carboxylic acid (650 mg, 93%) as a solid. LCMS (ES, m/z): 403 [M+H]⁺

### Synthesis of Intermediate C391

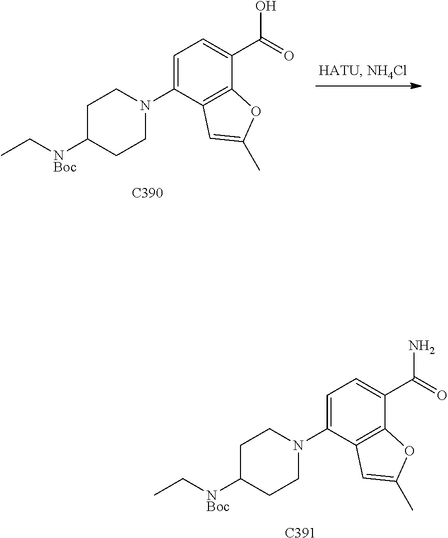

To a stirred solution of 4-{4-[(tert-butoxycarbonyl)(ethyl)amino]piperidin-1-yl}-2-methyl-1-benzofuran-7-carboxylic acid (650 mg, 1.615 mmol, 1 equiv) and DIEA (417.45 mg, 3.230 mmol, 2.0 equiv) in DMF (6 mL) were added HATU (736.87 mg, 1.938 mmol, 1.2 equiv) and NH₄Cl (856 mg, 16.15 mmol, 10.0 equiv) at room temperature. The resulting mixture was stirred for 2 h at room temperature. The reaction was quenched with water (50 mL) at room temperature. The precipitated solids were collected by filtration and washed with water (2×10 mL) to afford tert-butyl N-[1-(7-carbamoyl-2-methyl-1-benzofuran-4-yl)piperidin-4-yl]-N-ethylcarbamate (450 mg, 69%) as a solid. LCMS (ES, m/z): 402[M+H]⁺

### Synthesis of Intermediate C392

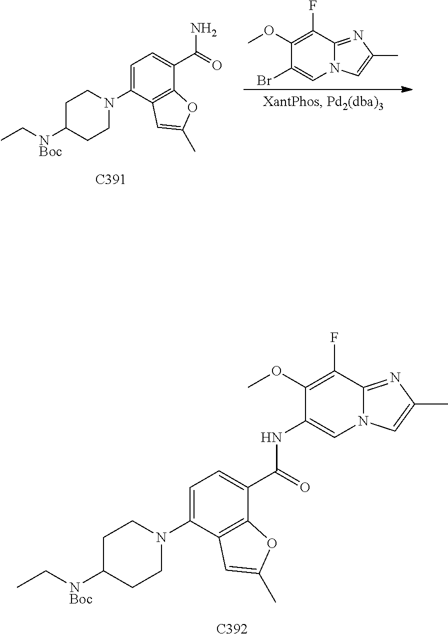

To a solution of 6-bromo-8-fluoro-7-methoxy-2-methylimidazo[1,2-a]pyridine (154.86 mg, 0.598 mmol, 1.2 equiv) and tert-butyl N-[1-(7-carbamoyl-2-methyl-1-benzofuran-4-yl)piperidin-4-yl]-N-ethylcarbamate (200 mg, 0.498 mmol, 1 equiv) in 1,4-dioxane (5 mL) were added Cs₂CO₃ (323.78 mg, 0.996 mmol, 2.0 equiv), RuPhos (23.24 mg, 0.050 mmol, 0.1 equiv) and 3rd Generation RuPhos precatalyst (41.66 mg, 0.050 mmol, 0.1 equiv). After stirring for 5 h at 90° C. under nitrogen atmosphere, the resulting mixture was concentrated under reduced pressure. The residue was purified by silica gel column chromatography, eluted with PE/EA (1:3) to afford tert-butyl N-ethyl-N-{1-[7-({8-fluoro-7-methoxy-2-methylimidazo[1,2-a]pyridin-6-yl}carbamoyl)-2-methyl-1-benzofuran-4-yl]piperidin-4-yl}carbamate (200 mg, 69%) as a solid. LCMS (ES, m/z): 580 [M+H]⁺

### Synthesis of Compound 501

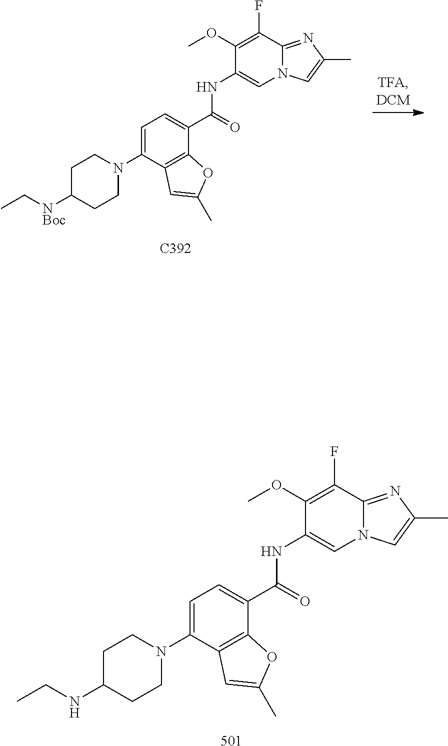

A solution of tert-butyl N-ethyl-N-{1-[7-({8-fluoro-7-methoxy-2-methylimidazo[1,2-a]pyridin-6-yl}carbamoyl)-2-methyl-1-benzofuran-4-yl]piperidin-4-yl}carbamate (100 mg, 0.173 mmol, 1 equiv) in TFA (3 mL, 40.389 mmol, 234.13 equiv) and DCM (3 mL) was stirred for 1 h at room temperature. The resulting mixture was concentrated under reduced pressure. The residue was basified to pH 8 with 7M NH₃(g) in MeOH. The resulting mixture was concentrated under vacuum. The residue was purified by reverse flash chromatography (Condition 6, Gradient 2) to afford 4-(4-(ethylamino)piperidin-1-yl)-N-(8-fluoro-7-methoxy-2-methylimidazo[1,2-a]pyridin-6-yl)-2-methylbenzofuran-7-carboxamide 2,2,2-trifluoroacetate (30 mg, 36%) as a solid. LCMS (ES, m/z): 480 [M+H]^{+ 1}H NMR (300 MHz, DMSO-d₆) δ 9.94 (s, 1H), 9.65 (d, J=1.1 Hz, 1H), 8.75 (s, 2H), 8.08 (dd, J=2.6, 1.3 Hz, 1H), 7.82 (d, J=8.5 Hz, 1H), 6.90-6.80 (m, 2H), 4.39 (d, J=4.0 Hz, 3H), 3.87 (d, J=12.6 Hz, 2H), 3.31 (s, 1H), 3.05 (q, J=6.8 Hz, 2H), 2.95 (t, J=12.3 Hz, 2H), 2.60 (s, 3H), 2.47-2.40 (m, 3H), 2.21-2.10 (m, 2H), 1.73 (td, J=12.8, 9.1 Hz, 2H), 1.24 (t, J=7.2 Hz, 3H).

### Example 239: Synthesis of Compound 502

### Synthesis of Intermediate C393

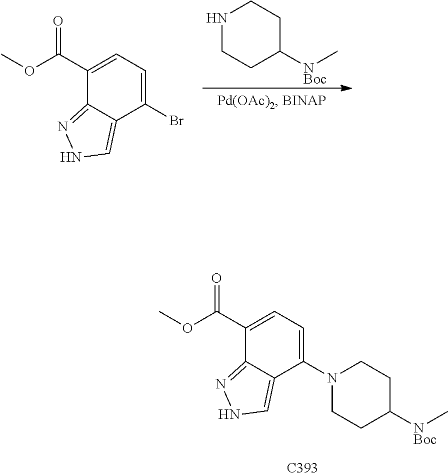

To a stirred solution of methyl 4-bromo-2H-indazole-7-carboxylate (1 g, 3.920 mmol, 1 equiv), K₂CO₃ (1.63 g, 11.760 mmol, 3 equiv) and tert-butyl N-methyl-N-(piperidin-4-yl)carbamate (1.68 g, 7.840 mmol, 2 equiv) in Toluene (10 mL) were added Pd(OAc)₂ (0.09 g, 0.392 mmol, 0.1 equiv) and BINAP (0.49 g, 0.784 mmol, 0.2 equiv) at room temperature under nitrogen atmosphere. The resulting mixture was stirred for overnight at 100° C. The resulting mixture was concentrated under reduced pressure. The residue was purified by silica gel column chromatography, eluted with PE/EA (3:1) to afford methyl 4-{4-[(tert-butoxycarbonyl)(methyl)amino]piperidin-1-yl}-2H-indazole-7-carboxylate (500 mg, 33%) as a solid. LCMS (ES, m/z): 389 [M+H]⁺

### Synthesis of Intermediate C394

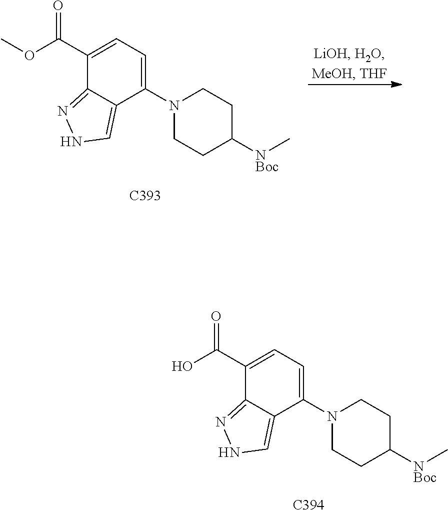

A solution of methyl 4-{4-[(tert-butoxycarbonyl)(methyl)amino]piperidin-1-yl}-2H-indazole-7-carboxylate (1 g, 2.574 mmol, 1 equiv) and lithiumol (0.31 g, 12.870 mmol, 5 equiv) in THF (5 mL), H₂O (5 mL) and MeOH (1 mL) was stirred for 3 h at 40° C. The resulting mixture was concentrated under reduced pressure. The mixture was acidified to pH 4 with HCl (1 mol/L, aq.). The precipitated solids were collected by filtration and washed with water (3×5 mL) to afford 4-{4-[(tert-butoxycarbonyl)(methyl)amino]piperidin-1-yl}-2H-indazole-7-carboxylic acid (600 mg, 62%) as a solid. LCMS (ES, m/z): 375 [M+H]⁺

### Synthesis of Intermediate C395

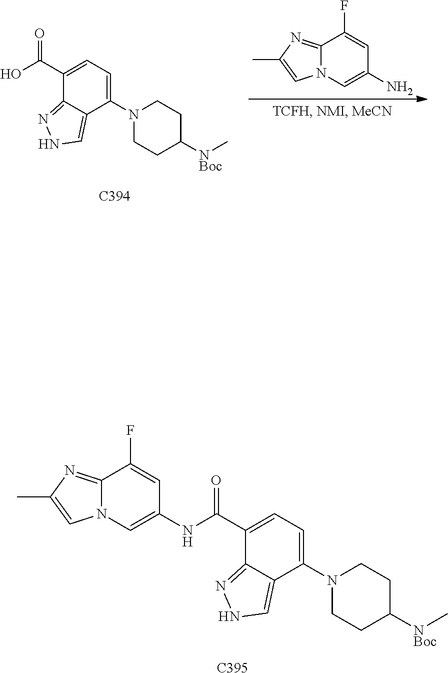

To a stirred solution of 4-{4-[(tert-butoxycarbonyl)(methyl)amino]piperidin-1-yl}-2H-indazole-7-carboxylic acid (500 mg, 1.335 mmol, 1 equiv) and 8-fluoro-2-methylimidazo[1,2-a]pyridin-6-amine (264.67 mg, 1.602 mmol, 1.2 equiv) in MeCN (10 mL) were added TCFH (487.06 mg, 1.736 mmol, 1.3 equiv) and NMI (383.73 mg, 4.672 mmol, 3.5 equiv) at room temperature. The resulting mixture was stirred for 3 h at room temperature. The resulting mixture was concentrated under reduced pressure. The residue was purified by silica gel column chromatography, eluted with PE/EA (0:1) to afford tert-butyl N-{1-[7-({8-fluoro-2-methylimidazo[1,2-a]pyridin-6-yl}carbamoyl)-2H-indazol-4-yl]piperidin-4-yl}-N-methylcarbamate (300 mg, 43%) as an oil. LCMS (ES, m/z): 522 [M+H]⁺

### Synthesis of Intermediate C396

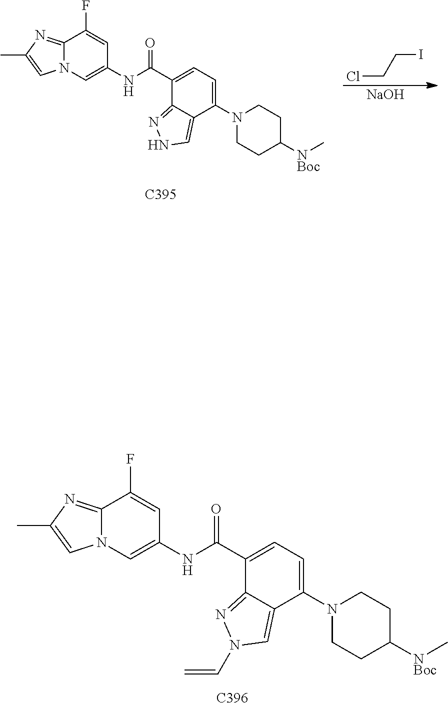

To a stirred solution of tert-butyl N-{1-[7-({8-fluoro-2-methylimidazo[1,2-a]pyridin-6-yl}carbamoyl)-2H-indazol-4-yl]piperidin-4-yl}-N-methylcarbamate (100 mg, 0.192 mmol, 1 equiv) and 1-chloro-2-iodoethane (55 mg, 0.288 mmol, 1.5 equiv) in DMF (5 mL) was added NaOH (39 mg, 0.960 mmol, 5 equiv) at room temperature. The resulting mixture was stirred for 16 h at room temperature. The reaction mixture was purified by reverse flash chromatography (Condition 3, Gradient 9) to afford tert-butyl N-{1-[2-ethenyl-7-({8-fluoro-2-methylimidazo[1,2-a]pyridin-6-yl}carbamoyl) indazol-4-yl]piperidin-4-yl}-N-methylcarbamate (60 mg, 57%) as a solid. LCMS (ES, m/z): 548 [M+H]⁺

### Synthesis of Compound 502

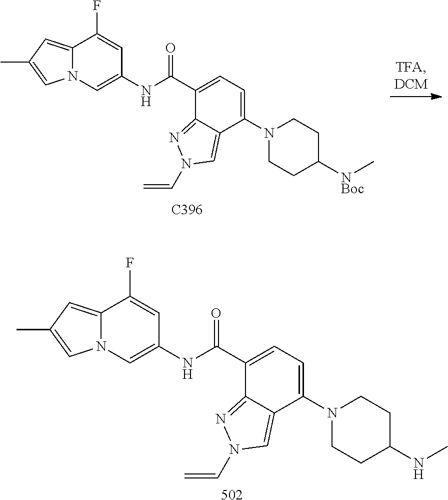

A solution of tert-butyl N-{1-[2-ethenyl-7-({8-fluoro-2-methylimidazo[1,2-a]pyridin-6-yl}carbamoyl) indazol-4-yl]piperidin-4-yl}-N-methylcarbamate (60 mg, 0.109 mmol, 1 equiv) and trifluoroacetic acid (2 mL) in DCM (5 mL) was stirred for 2 h at room temperature. The resulting mixture was concentrated under reduced pressure. The residue was purified by reverse flash chromatography (Condition 3, Gradient 10) to afford 2-ethenyl-N-{8-fluoro-2-methylimidazo[1,2-a]pyridin-6-yl}-4-[4-(methylamino)piperidin-1-yl]indazole-7-carboxamide (11 mg, 22%) as a solid. LCMS (ES, m/z): 448 [M+H]^{+ 1}H NMR (300 MHz, DMSO-d₆) δ 10.91 (s, 1H), 9.22 (s, 1H), 9.08 (s, 1H), 8.03 (d, J=8.2 Hz, 1H), 7.91 (d, J=3.3 Hz, 1H), 7.68 (dd, J=15.5, 8.7 Hz, 1H), 7.35 (d, J=12.8 Hz, 1H), 6.52 (d, J=8.1 Hz, 1H), 6.26 (d, J=15.7 Hz, 1H), 5.36 (d, J=8.5 Hz, 1H), 3.93 (d, J=12.7 Hz, 2H), 3.11 (t, J=11.9 Hz, 2H), 2.65 (s, 1H), 2.36 (d, J=1.7 Hz, 6H), 1.99 (d, J=13.2 Hz, 2H), 1.48 (d, J=11.0 Hz, 2H).

### Example 240: Synthesis of Compound 503

### Synthesis of Intermediate C397

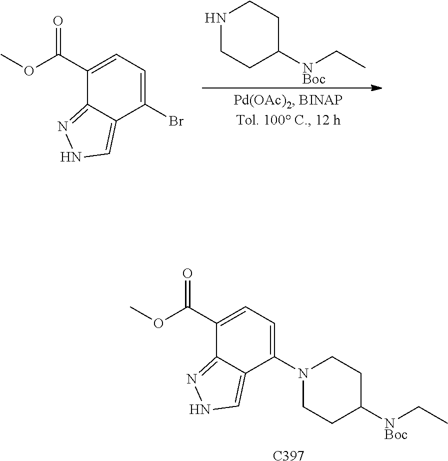 [SMILES: COC(=O)c1ccc(Br)c2c[nH]nc12 ; CCN(C(=O)OC(C)(C)C)C1CCNCC1 ; CCN(C(=O)OC(C)(C)C)C1CCN(c2ccc(C(=O)OC)c3n[nH]cc23)CC1 ; "C397"]

To a solution of methyl 4-bromo-2H-indazole-7-carboxylate (1 g, 3.920 mmol, 1 equiv) and tert-butyl N-ethyl-N-(piperidin-4-yl)carbamate (1.79 g, 7.840 mmol, 2.0 equiv) in Toluene (20 mL) were added K₂CO₃ (1.63 g, 11.760 mmol, 3.0 equiv), BINAP (0.49 g, 0.784 mmol, 0.2 equiv) and Pd(OAc)₂ (0.09 g, 0.392 mmol, 0.1 equiv). After stirring for 12 h at 100° C. under a nitrogen atmosphere, the resulting mixture was concentrated under reduced pressure. The residue was purified by silica gel column chromatography, eluted with PE/EA (1:1) to afford methyl 4-{4-[(tert-butoxycarbonyl)(ethyl)amino]piperidin-1-yl}-2H-indazole-7-carboxylate (1 g, 63%) as a solid. LCMS (ES, m/z): 403 [M+H]⁺

### Synthesis of Intermediate C398

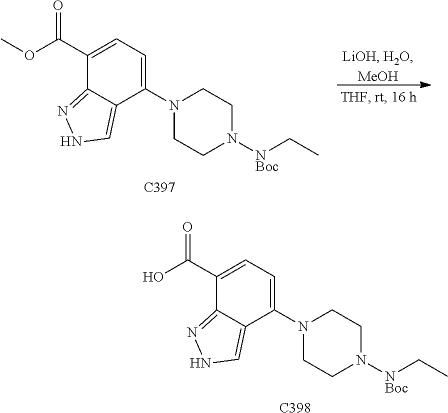

To a stirred solution of methyl 4-{4-[(tert-butoxycarbonyl)(ethyl)amino]piperidin-1-yl}-2H-indazole-7-carboxylate (1 g, 2.485 mmol, 1 equiv) in THF (9 mg) and MeOH (9 mL) was added a solution of LiOH·H₂O (0.6 g, 24.850 mmol, 10 equiv) in H₂O (9 mL) dropwise at room temperature. The resulting mixture was stirred for 1 h at room temperature. The mixture was acidified to pH 2 with 2 M HCl. The precipitated solids were collected by filtration and washed with H₂O (1×10 mL). This resulted in 4-{4-[(tert-butoxycarbonyl)(ethyl)amino]piperidin-1-yl}-2H-indazole-7-carboxylic acid (0.8 g, 83%) as a solid. LCMS (ES, m/z): 389[M+H]⁺

### Synthesis of Intermediate C399

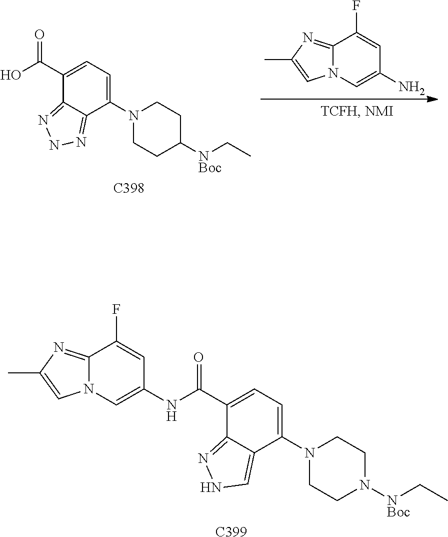 [SMILES: CCN(C(=O)OC(C)(C)C)C1CCN(c2ccc(C(=O)O)c3n[nH]nc23)CC1 ; Cc1cn2cc(N)cc(F)c2n1 ; "C398" ; CCN(C(=O)OC(C)(C)C)N1CCN(c2ccc(C(=O)Nc3cc(F)c4nc(C)cn4c3)c3n[nH]cc23)CC1 ; "C399"]

To a stirred solution of 4-{4-[(tert-butoxycarbonyl)(ethyl)amino]piperidin-1-yl}-2H-indazole-7-carboxylic acid (300 mg, 0.772 mmol, 1 equiv) and 8-fluoro-2-methylimidazo[1,2-a]pyridin-6-amine (191.3 mg, 1.158 mmol, 1.5 equiv) in CH₃CN (6 mL) were added TCFH (281.6 mg, 1.004 mmol, 1.3 equiv) and NMI (221.9 mg, 2.702 mmol, 3.5 equiv) dropwise at room temperature. The resulting mixture was stirred for 1 h at room temperature. The resulting mixture was concentrated under reduced pressure. The residue was purified by reverse flash chromatography (Condition 3, Gradient 8) to afford tert-butyl N-ethyl-N-{1-[7-({8-fluoro-2-methylimidazo[1,2-a]pyridin-6-yl}carbamoyl)-2H-indazol-4-yl]piperidin-4-yl}carbamate (183 mg, 44%) as a solid. LCMS (ES, m/z): 536[M+H]⁺

### Synthesis of Intermediate C400

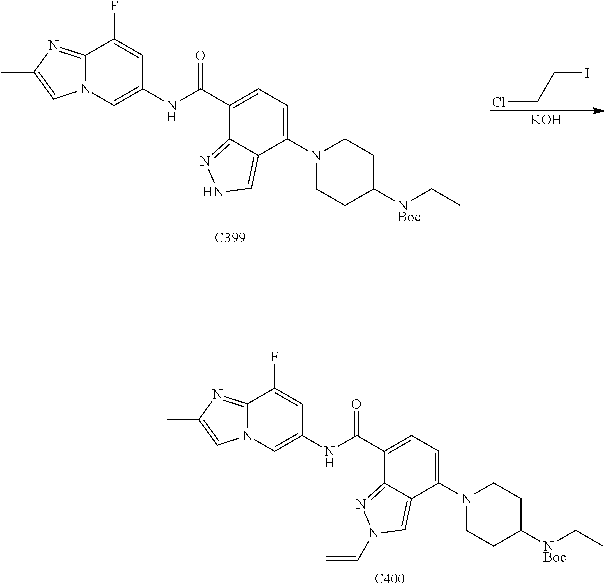

To a stirred solution of tert-butyl N-ethyl-N-{1-[7-({8-fluoro-2-methylimidazo[1,2-a]pyridin-6-yl}carbamoyl)-2H-indazol-4-yl]piperidin-4-yl}carbamate (180 mg, 0.336 mmol, 1 equiv) and 1-chloro-2-iodoethane (95.9 mg, 0.504 mmol, 1.5 equiv) in DMF (3 mL) was added KOH (113.1 mg, 2.016 mmol, 6.0 equiv) in portions at room temperature. The resulting mixture was stirred for 12 h at 80° C. The mixture was allowed to cool down to room temperature. The resulting mixture was purified by reverse flash chromatography (Condition 5, Gradient 2) to afford tert-butyl N-{1-[2-ethenyl-7-({8-fluoro-2-methylimidazo[1,2-a]pyridin-6-yl}carbamoyl) indazol-4-yl]piperidin-4-yl}-N-ethylcarbamate (80 mg, 42%) as a solid. LCMS (ES, m/z): 562[M+H]⁺

### Synthesis of Compound 503

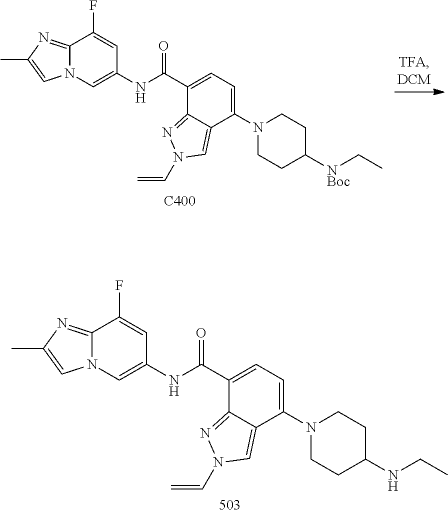

A solution of tert-butyl N-{1-[2-ethenyl-7-({8-fluoro-2-methylimidazo[1,2-a]pyridin-6-yl}carbamoyl) indazol-4-yl]piperidin-4-yl}-N-ethylcarbamate (80 mg, 0.142 mmol, 1 equiv) and TFA (0.2 mL) in DCM (2 mL) was stirred for 1 h at room temperature. The mixture was basified to pH 8 with NH₃(g) in MeOH. The resulting mixture was concentrated under reduced pressure. The residue was purified by reverse flash chromatography (Condition 3, Gradient 10) to afford 2-ethenyl-4-[4-(ethylamino)piperidin-1-yl]-N-{8-fluoro-2-methylimidazo[1,2-a]pyridin-6-yl}indazole-7-carboxamide (18 mg, 27%) as a solid. LCMS (ES, m/z): 462[M+H]^{+ 1}H NMR (300 MHz, DMSO-d₆) δ 9.15 (t, J=1.7 Hz, 1H), 9.01 (d, J=1.5 Hz, 1H), 8.01 (d, J=8.1 Hz, 1H), 7.87 (d, J=3.1 Hz, 1H), 7.64 (dd, J=15.5, 8.7 Hz, 1H), 7.32 (dd, J=12.3, 1.5 Hz, 1H), 6.50 (d, J=8.2 Hz, 1H), 6.22 (d, J=15.5 Hz, 1H), 5.34 (d, J=8.5 Hz, 1H), 3.94 (d, J=12.9 Hz, 2H), 3.06 (t, J=12.1 Hz, 2H), 2.80-2.71 (m, 1H), 2.64 (q, J=7.1 Hz, 2H), 2.34 (s, 3H), 1.99 (d, J=12.6 Hz, 2H), 1.48 (q, J=11.4 Hz, 2H), 1.06 (t, J=7.1 Hz, 3H).

### Example 241: Synthesis of Compound 504

### Synthesis of Intermediate C402

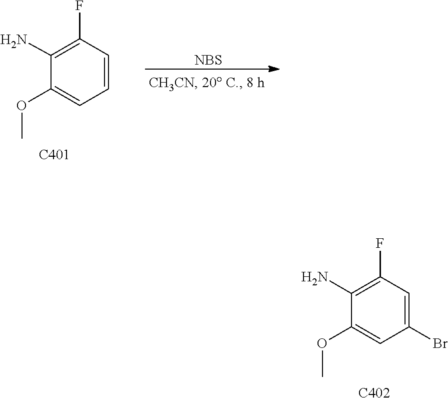

A solution of 2-fluoro-6-methoxyaniline (15 g, 106.274 mmol, 1 equiv) in CH₃CN (200 mL) was treated with NBS (28.37 g, 159.411 mmol, 1.5 equiv) for 8 hours at 20° C. The mixture was neutralized to pH 7 with NaHCO₃. The aqueous layer was extracted with DCM 500 mL×3 times. The combined organic layers were washed with water (1×500 mL), dried over anhydrous Na₂SO₄. After filtration, the filtrate was concentrated under reduced pressure. The residue was purified by silica gel column chromatography, eluted with PE/EA (3:1) to afford 4-bromo-2-fluoro-6-methoxyaniline (5 g, 21%) is given with solid. LCMS (ES, m/z): 220 [M+H]⁺

### Synthesis of Intermediate C403

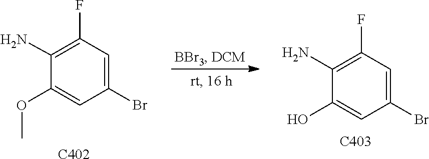

To a solution of 4-bromo-2-fluoro-6-methoxyaniline (5 g, 22.723 mmol, 1 equiv) in DCM (50 mL) was added BBr₃ (8.54 g, 34.084 mmol, 1.5 equiv) dropwise at 0° C. The resulting mixture was stirred for 16 h at room temperature. The resulting mixture was diluted with water (100 mL). The mixture was neutralized to pH 7 with NaHCO₃. The resulting mixture was extracted with EtOAc (3×200 mL). The combined organic layers were washed with water (1×500 mL), dried over anhydrous Na₂SO₄. After filtration, the filtrate was concentrated under reduced pressure. The residue was purified by silica gel column chromatography, eluted with MeOH/DCM (1/20) to afford 2-amino-5-bromo-3-fluorophenol (3.7 g, 79%) as an oil. LCMS (ES, m/z): 206 [M+H]⁺

### Synthesis of Intermediate C404

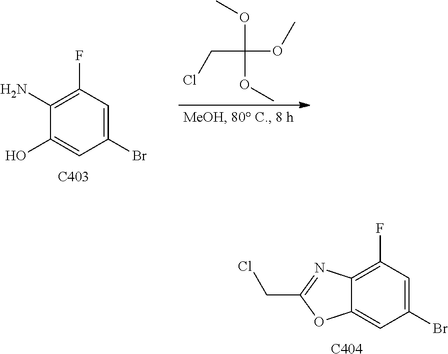

A solution of 2-amino-5-bromo-3-fluorophenol (4 g, 19.416 mmol, 1 equiv) in MeOH (100 mL) was treated with 2-chloro-1,1,1-trimethoxyethane (6.00 g, 38.832 mmol, 2.00 equiv) for 8 hours at 80° C. The resulting mixture was concentrated under reduced pressure. The residue was purified by silica gel column chromatography, eluted with PE/EA (3/1) to afford 6-bromo-2-(chloromethyl)-4-fluoro-1,3-benzoxazole (4.5 g, 88%) as a solid. LCMS (ES, m/z): 264 [M+H]⁺

### Synthesis of Intermediate C405

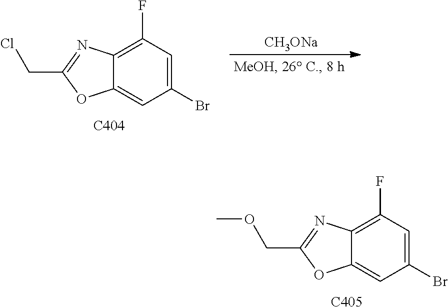

A solution of 6-bromo-2-(chloromethyl)-4-fluoro-1,3-benzoxazole (2.26 g, 8.545 mmol, 1 equiv) in MeOH (20 mL) was treated with sodium methoxide (4.62 g, 85.450 mmol, 10 equiv) for 8 hours at 26° C. The resulting mixture was diluted with water (100 mL). The aqueous layer was extracted with DCM (3×100 mL). The resulting mixture was washed with 2×200 mL of brine. The resulting organic layer was dried by Na₂SO₄ and concentrated under reduced pressure. The residue was purified by silica gel column chromatography, eluted with PE/EA (3/1) to afford 6-bromo-4-fluoro-2-(methoxymethyl)-1,3-benzoxazole (2 g, 90%) as a solid. LCMS (ES, m/z): 260 [M+H]⁺

### Synthesis of Intermediate C406

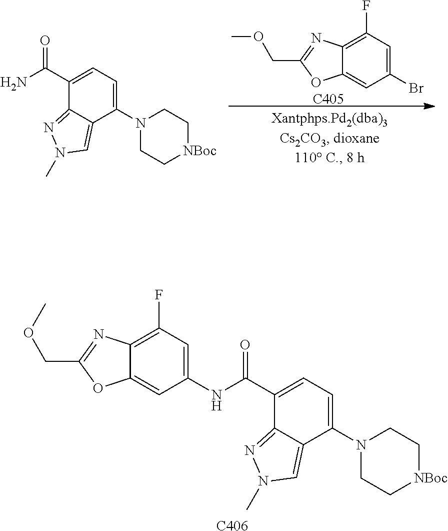

A solution of 6-bromo-4-fluoro-2-(methoxymethyl)-1,3-benzoxazole (200 mg, 0.769 mmol, 1 equiv) in dioxane (5 mL) was treated with tert-butyl 4-(7-carbamoyl-2-methylindazol-4-yl)piperazine-1-carboxylate (360 mg, 1.002 mmol, 1.30 equiv), Pd₂(dba)₃ (70 mg, 0.076 mmol, 0.10 equiv), Cs₂CO₃ (501 mg, 1.538 mmol, 2.00 equiv), XantPhos (89 mg, 0.154 mmol, 0.20 equiv) for 8 hours at 110° C. under nitrogen atmosphere. The resulting mixture was concentrated under vacuum. The residue was purified by silica gel column chromatography, eluted with MeOH/DCM (1/10) to afford tert-butyl 4-(7-{[4-fluoro-2-(methoxymethyl)-1,3-benzoxazol-6-yl]carbamoyl}-2-methylindazol-4-yl)piperazine-1-carboxylate (380 mg, 91%) as a solid. LCMS (ES, m/z): 539 [M+H]⁺

### Synthesis of Compound 504

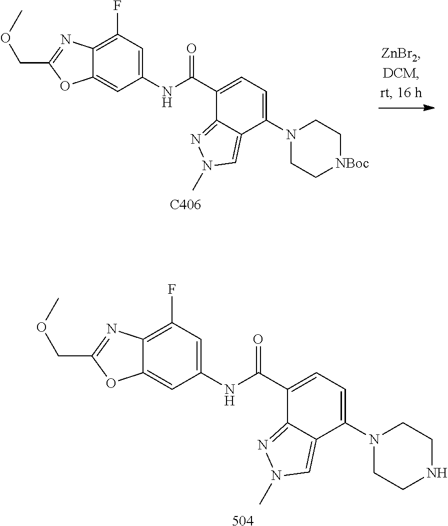

To a stirred mixture of tert-butyl 4-(7-{[4-fluoro-2-(methoxymethyl)-1,3-benzoxazol-6-yl]carbamoyl}-2-methylindazol-4-yl)piperazine-1-carboxylate (80 mg, 0.149 mmol, 1 equiv) in DCM (2 mL) was added ZnBr₂ (194.2 mg, 2.980 mmol, 20 equiv) at room temperature. The resulting mixture was stirred for 16 h at room temperature. The resulting mixture was concentrated under vacuum. The crude product was purified by Prep-HPLC (Condition 10, Gradient 7) to afford N-[4-fluoro-2-(methoxymethyl)-1,3-benzoxazol-6-yl]-2-methyl-4-(piperazin-1-yl) indazole-7-carboxamide (8 mg, 12%) as a solid. LCMS (ES, m/z): 439 [M+H]^{+ 1}H NMR (400 MHz, DMSO-d₆) δ 11.24 (s, 1H), 8.78 (s, 1H), 7.96 (d, J=8.1 Hz, 1H), 7.41 (dd, J=12.2, 2.3 Hz, 1H), 7.24 (t, J=1.8 Hz, 1H), 6.47 (d, J=8.2 Hz, 1H), 4.73 (s, 2H), 4.28 (s, 3H), 3.91 (s, 3H), 3.38-3.32 (m, 4H), 2.94-2.87 (m, 4H).

### Example 242: Synthesis of Compound 505

### Synthesis of Intermediate C407

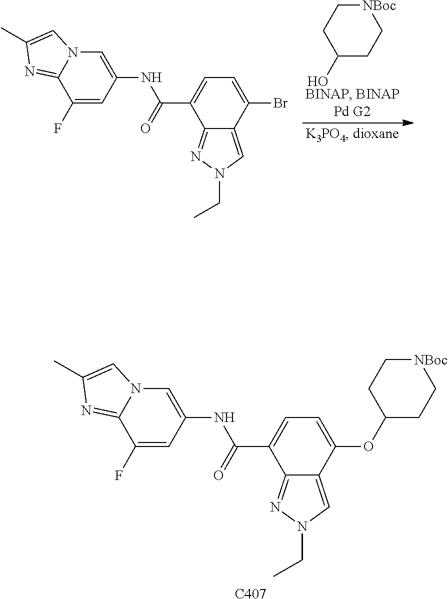

To a solution of 4-bromo-2-ethyl-N-{8-fluoro-2-methylimidazo[1,2-a]pyridin-6-yl}indazole-7-carboxamide (200 mg, 0.480 mmol, 1 equiv) and tert-butyl 4-hydroxypiperidine-1-carboxylate (290.1 mg, 1.440 mmol, 3.0 equiv) in dioxane (0.5 mL) were added K₃PO₄ (305.9 mg, 1.440 mmol, 3.0 equiv), BINAP (29.9 mg, 0.048 mmol, 0.1 equiv) and Binap Palladacycle Gen. 2 (44.8 mg, 0.048 mmol, 0.1 equiv). After stirring for 12 h at 100° C. under a nitrogen atmosphere, the resulting mixture was concentrated under reduced pressure. The residue was purified by reverse flash chromatography (Condition 3, Gradient 8) to afford tert-butyl 4-{[2-ethyl-7-({8-fluoro-2-methylimidazo[1,2-a]pyridin-6-yl}carbamoyl) indazol-4-yl]oxy}piperidine-1-carboxylate (100 mg, 39%) as a solid. LCMS (ES, m/z): 537 [M+H]⁺

### Synthesis of Compound 505

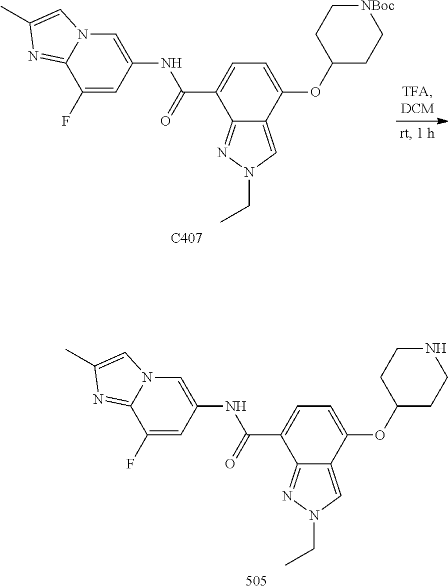

A solution of tert-butyl 4-{[2-ethyl-7-({8-fluoro-2-methylimidazo[1,2-a]pyridin-6-yl}carbamoyl) indazol-4-yl]oxy}piperidine-1-carboxylate (100 mg, 0.186 mmol, 1 equiv) and TFA (0.2 mL) in DCM (2 mL) was stirred for 1 h at room temperature. The mixture was basified to pH 8 with NH₃(g) in MeOH. The resulting mixture was concentrated under reduced pressure. The residue was purified by reverse flash chromatography (Condition 3, Gradient 2) to afford 2-ethyl-N-{8-fluoro-2-methylimidazo[1,2-a]pyridin-6-yl}-4-(piperidin-4-yloxy) indazole-7-carboxamide (45 mg, 55%) as a solid. LCMS (ES, m/z): 437 [M+H]^{+ 1}H NMR (300 MHz, DMSO-d₆) δ 10.95 (s, 1H), 9.21 (d, J=1.6 Hz, 1H), 8.72 (s, 1H), 8.05 (d, J=8.0 Hz, 1H), 7.92 (d, J=3.1 Hz, 1H), 7.33 (dd, J=12.3, 1.7 Hz, 1H), 6.74 (d, J=8.2 Hz, 1H), 4.73 (d, J=8.7 Hz, 1H), 4.61 (q, J=7.3 Hz, 2H), 3.01 (d, J=12.8 Hz, 2H), 2.65 (t, J=9.9 Hz, 2H), 2.39-2.33 (m, 3H), 2.03 (d, J=12.0 Hz, 2H), 1.61 (t, J=7.3 Hz, 5H).

### Example 243: Synthesis of Compound 506

### Synthesis of Intermediate C408

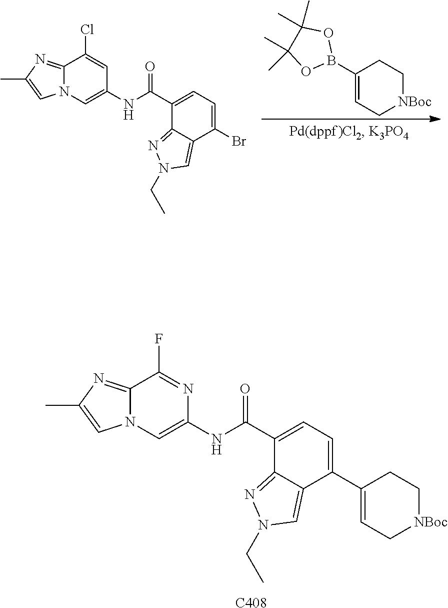

To a solution of 4-bromo-2-ethyl-N-{8-fluoro-2-methylimidazo[1,2-a]pyridin-6-yl}indazole-7-carboxamide (100 mg, 0.240 mmol, 1 equiv) and tert-butyl 4-(4,4,5,5-tetramethyl-1,3,2-dioxaborolan-2-yl)-3,6-dihydro-2H-pyridine-1-carboxylate (89.1 mg, 0.288 mmol, 1.2 equiv) in dioxane (2 mL) were added K₃PO₄ (152.9 mg, 0.720 mmol, 3.0 equiv) and Pd(dppf)Cl₂CH₂Cl₂ (19.5 mg, 0.024 mmol, 0.1 equiv). After stirring for 1 h at 80° C. under a nitrogen atmosphere, the resulting mixture was concentrated under reduced pressure. The residue was purified by reverse flash chromatography (Condition 5, Gradient 2) to afford tert-butyl 4-[2-ethyl-7-({8-fluoro-2-methylimidazo[1,2-a]pyridin-6-yl}carbamoyl) indazol-4-yl]-3,6-dihydro-2H-pyridine-1-carboxylate (100 mg, 80%) as a solid. LCMS (ES, m/z): 519 [M+H]⁺

### Synthesis of Compound 506

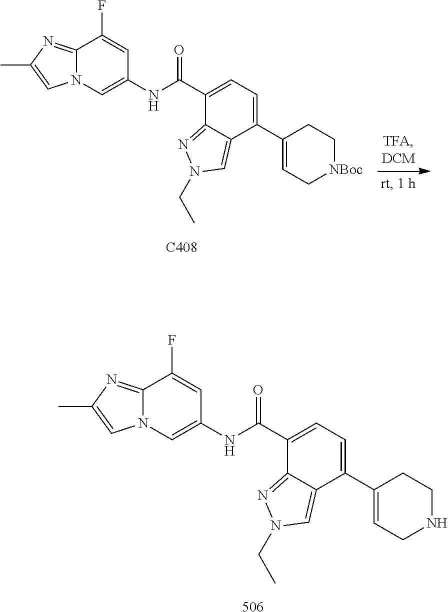

A solution of tert-butyl 4-[2-ethyl-7-({8-fluoro-2-methylimidazo[1,2-a]pyridin-6-yl}carbamoyl) indazol-4-yl]-3,6-dihydro-2H-pyridine-1-carboxylate (80 mg, 0.154 mmol, 1 equiv) and TFA (0.2 mL) in DCM (2 mL) was stirred for 1 h at room temperature. The mixture was basified to pH 8 with NH₃(g) in MeOH. The resulting mixture was concentrated under reduced pressure. The residue was purified by reverse flash chromatography (Condition 3, Gradient 2) to afford 2-ethyl-N-{8-fluoro-2-methylimidazo[1,2-a]pyridin-6-yl}-4-(1,2,3,6-tetrahydropyridin-4-yl) indazole-7-carboxamide (25 mg, 38%) as a solid. LCMS (ES, m/z): 419[M+H]^{+ 1}H NMR (300 MHz, DMSO-d₆) δ 11.34 (s, 1H), 9.43 (s, 1H), 8.98 (d, J=14.9 Hz, 3H), 8.12 (s, 1H), 8.12 (d, J=7.5 Hz, 1H), 7.72 (s, 1H), 7.32 (d, J=7.5 Hz, 1H), 6.47 (s, 1H), 4.69 (q, J=7.3 Hz, 2H), 3.90 (s, 2H), 2.83 (s, 2H), 2.83 (s, 2H), 2.43 (t, J=1.1 Hz, 3H), 1.65 (t, J=7.3 Hz, 3H).

### Example 244: Synthesis of Compound 507

### Synthesis of Intermediate C410

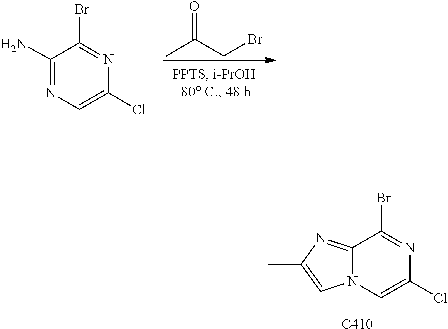

A solution of 3-bromo-5-chloropyrazin-2-amine (1 g, 4.798 mmol, 1.0 equiv) in isopropanol (30 mL) was treated with PPTS (120 mg, 0.480 mmol, 0.1 equiv) and bromoacetone (1.97 g, 14.394 mmol, 3.0 equiv) for 48 hours at 80° C. The resulting mixture was concentrated under reduced pressure. The residue was purified by silica gel column chromatography, eluted with PE/EA (50%) to afford 8-bromo-6-chloro-2-methylimidazo[1,2-a]pyrazine (600 mg, 51%) as a solid. LCMS (ES, m/z): 246 [M+H]⁺

### Synthesis of Intermediate C411

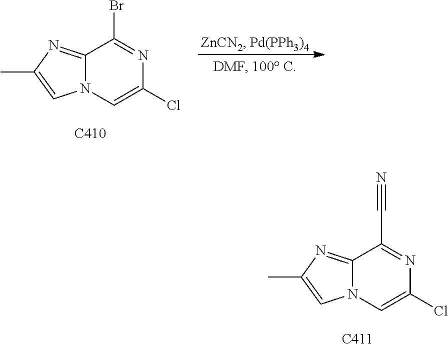

To a solution of 8-bromo-6-chloro-2-methylimidazo[1,2-a]pyrazine (450 mg, 1.826 mmol, 1 equiv) in DMF (5 mL) were added Zn(CN)₂ (1071 mg, 9.130 mmol, 5.0 equiv) and Pd(PPh₃)₄ (210 mg, 0.183 mmol, 0.1 equiv). After stirring for 16 h at 100° C. under a nitrogen atmosphere, the resulting mixture was concentrated under reduced pressure. The residue was purified by silica gel column chromatography, eluted with DCM/MeOH (10:1) to afford 6-chloro-2-methylimidazo[1,2-a]pyrazine-8-carbonitrile (200 mg, 57%) as a solid. LCMS (ES, m/z): 193 [M+H]^{+ 1}H NMR (300 MHz, Chloroform-d) δ 8.31 (s, 1H), 7.69 (s, 1H), 2.64 (d, J=0.7 Hz, 3H).

### Synthesis of Intermediate C412

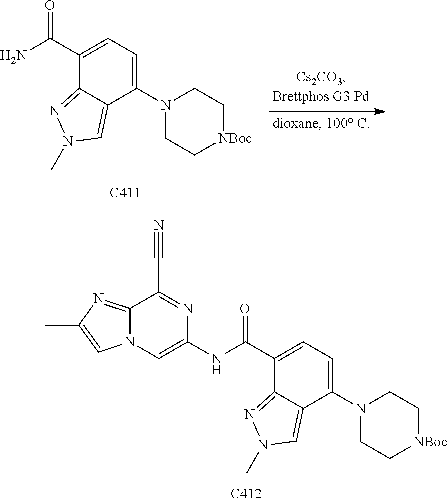

To a stirred mixture of tert-butyl 4-(7-carbamoyl-2-methylindazol-4-yl)piperazine-1-carboxylate (150 mg, 0.417 mmol, 1.0 equiv) and 6-bromo-2-methylimidazo[1,2-a]pyrazine-8-carbonitrile (98.9 mg, 0.417 mmol, 1.0 equiv) in dioxane (3 mL) were added Cs₂CO₃ (407.9 mg, 1.251 mmol, 3.0 equiv) at room temperature under nitrogen atmosphere. The resulting mixture was stirred for 16 h at 100° C. under nitrogen atmosphere. The resulting mixture was concentrated under vacuum. The residue was purified by silica gel column chromatography, eluted with PE/EA (1:9) to afford tert-butyl 4-[7-({8-cyano-2-methylimidazo[1,2-a]pyrazin-6-yl}carbamoyl)-2-methylindazol-4-yl]piperazine-1-carboxylate (40 mg, 19%) as a solid. LCMS (ES, m/z): 516 [M+H]⁺

### Synthesis of Compound 507

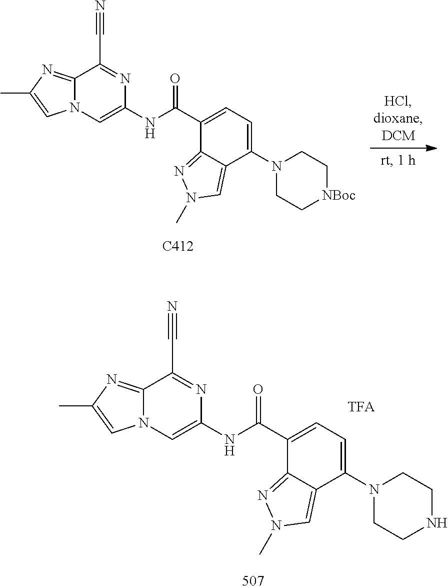

To a stirred mixture of tert-butyl 4-[7-({8-cyano-2-methylimidazo[1,2-a]pyrazin-6-yl}carbamoyl)-2-methylindazol-4-yl]piperazine-1-carboxylate (30 mg, 0.058 mmol, 1 equiv) in DCM (1 mL) was added HCl(gas) in 1,4-dioxane (0.25 mL, 4M) dropwise at room temperature under nitrogen atmosphere. The resulting mixture was concentrated under vacuum. The crude product was purified by Prep-HPLC (Condition 14, Gradient 2) to afford N-{8-cyano-2-methylimidazo[1,2-a]pyrazin-6-yl}-2-methyl-4-(piperazin-1-yl) indazole-7-carboxamide (8.6 mg, 36%) as a solid. LCMS (ES, m/z): 416 [M+H]^{+ 1}H NMR (400 MHz, DMSO-d₆) δ 11.51 (s, 1H), 9.79 (s, 1H), 8.93 (s, 1H), 8.84 (s, 2H), 8.32 (s, 1H), 8.10 (d, J=8.0 Hz, 1H), 6.64 (d, J=8.1 Hz, 1H), 4.32 (s, 3H), 3.62 (t, J=5.1 Hz, 4H), 3.36 (d, J=5.1 Hz, 4H), 2.50 (s, 3H).

### Example 245: Synthesis of Compound 508

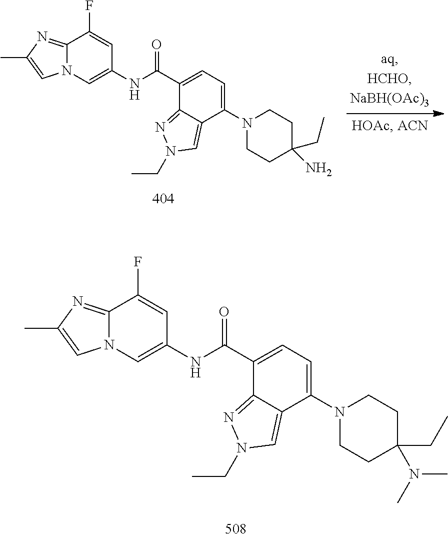

To a stirred solution of 4-(4-amino-4-ethylpiperidin-1-yl)-2-ethyl-N-{8-fluoro-2-methylimidazo[1,2-a]pyridin-6-yl}indazole-7-carboxamide (130 mg, 0.28 mmol, 1 equiv) and aq. HCHO (105 mg, 1.4 mmol, 5 equiv, 40% HCHO in water) in MeCN (5 mL) was added NaBH(OAc)₃ (178 mg, 0.84 mmol, 3 equiv) at room temperature. The resulting mixture was stirred for 0.5 h at room temperature. To the above mixture was added HOAc (170 mg, 2.8 mmol, 10 equiv) dropwise at room temperature. The resulting mixture was stirred for additional 3 h at room temperature. The reaction was quenched by the addition of water (5 mL) at room temperature. The resulting mixture was concentrated under vacuum. The residue was purified by reverse flash chromatography (Condition 3, Gradient 2) to afford 4-[4-(dimethylamino)-4-ethylpiperidin-1-yl]-2-ethyl-N-{8-fluoro-2-methylimidazo[1,2-a]pyridin-6-yl}indazole-7-carboxamide (42.2 mg, 31%) as a solid. LCMS (ES, m/z): 492 [M+H]^{+ 1}H NMR (400 MHz, DMSO-d₆) δ 11.10 (s, 1H), 9.20 (d, J=1.7 Hz, 1H), 8.82 (s, 1H), 7.96 (d, J=8.1 Hz, 1H), 7.89 (d, J=3.1 Hz, 1H), 7.27 (dd, J=12.3, 1.7 Hz, 1H), 6.43 (d, J=8.3 Hz, 1H), 4.58 (q, J=7.3 Hz, 2H), 3.53 (dt, J=11.8, 4.5 Hz, 2H), 3.41 (ddd, J=12.4, 9.9, 2.9 Hz, 2H), 2.35 (s, 3H), 2.23 (s, 6H), 1.91-1.77 (m, 2H), 1.70-1.54 (m, 5H), 1.50 (q, J=7.5 Hz, 2H), 0.85 (t, J=7.5 Hz, 3H).

### Example 246: Synthesis of Compound 510

### Synthesis of Intermediate C413

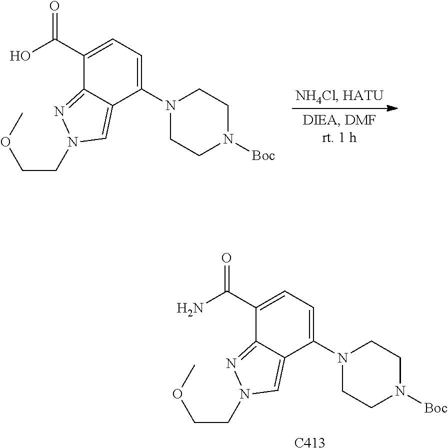

A solution of 4-[4-(tert-butoxycarbonyl) piperazin-1-yl]-2-(2-methoxyethyl) indazole-7-carboxylic acid (480 mg, 1.187 mmol, 1 equiv) in DMF (1 mL) was added NH₄Cl (317.4 mg, 5.935 mmol, 5.0 equiv), HATU (676.8 mg, 1.780 mmol, 1.5 equiv) and DIEA (460.1 mg, 3.561 mmol, 3.0 equiv). The mixture was stirred at room temperature for 1 h and washed with 2×10 mL of water, extracted with EtOAc (3×20 mL), and dried over anhydrous Na₂SO₄. After filtration, the filtrate was concentrated under reduced pressure. The residue was purified by silica gel column chromatography, eluted with PE/EA (65%) to afford tert-butyl 4-[7-carbamoyl-2-(2-methoxyethyl) indazol-4-yl] piperazine-1-carboxylate (520 mg, 99%) as an oil. LCMS (ES, m/z): 404 [M+H]⁺

### Synthesis of Intermediate C414

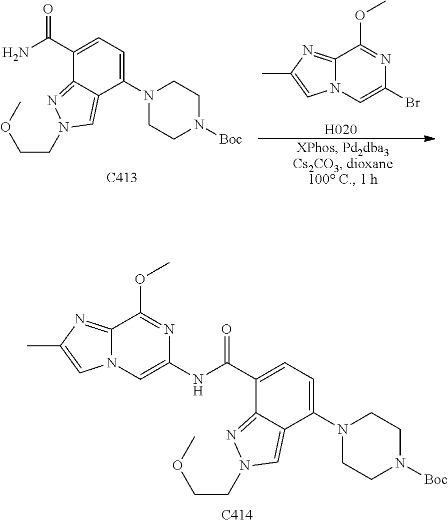

A solution of tert-butyl 4-[7-carbamoyl-2-(2-methoxyethyl) indazol-4-yl] piperazine-1-carboxylate (200 mg, 0.496 mmol, 1 equiv) in dioxane (3 mL) was added 6-bromo-8-methoxy-2-methylimidazo[1,2-a] pyrazine (179.9 mg, 0.744 mmol, 1.5 equiv), Cs₂CO₃ (323.0 mg, 0.992 mmol, 2 equiv), XantPhos (57.3 mg, 0.099 mmol, 0.2 equiv) and Pd₂(dba)₃ (45.3 mg, 0.050 mmol, 0.1 equiv) under nitrogen atmosphere. The mixture was stirred at 100° C. for 1 h to give a black solution. The resulting mixture was concentrated under vacuum. The residue was purified by silica gel column chromatography, eluted with PE/EA (95%) to afford N-{8-methoxy-2-methylimidazo[1,2-a]pyrazin-6-yl}-2-(2-methoxyethyl)-4-(piperazin-1-yl) indazole-7-carboxamide (100 mg, 43%) as a oil. LCMS (ES, m/z): 565 [M+H]⁺

### Synthesis of Compound 510

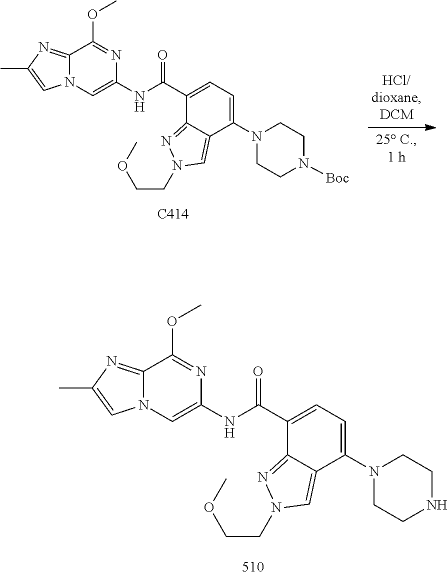

A solution of tert-butyl 4-[7-({8-methoxy-2-methylimidazo[1,2-a] pyrazin-6-yl}carbamoyl)-2-(2-methoxyethyl) indazol-4-yl] piperazine-1-carboxylate (100 mg, 0.177 mmol, 1 equiv) in DCM (3 mL) was added HCl (gas) in 1,4-dioxane (1.5 mL, 4M). The mixture was stirred for 1 h at 25° C. The resulting mixture was concentrated under vacuum. The crude product was purified by Prep-HPLC with the following conditions (SunFire Prep C18 OBD Column 19*150 mm, 5 μm 10 nm, mobile phase, MeCN in water (0.05% TFA), 20% to 40% gradient in 7 min; detector, UV 254 nm) to afford N-{8-methoxy-2-methylimidazo[1,2-a]pyrazin-6-yl}-2-(2-methoxyethyl)-4-(piperazin-1-yl) indazole-7-carboxamide (36.5 mg, 44%) as a solid. LCMS (ES, m/z): 465 [M+H]^{+ 1}H NMR (400 MHz, DMSO-d₆) δ 11.48 (d, J=6.6 Hz, 1H), 9.10 (d, J=7.6 Hz, 1H), 8.94 (s, 1H), 8.83-8.80 (m, 1H), 8.06-8.00 (m, 2H), 6.64 (d, J=8.1 Hz, 1H), 4.70 (t, J=5.1 Hz, 2H), 4.15 (d, J=2.2 Hz, 4H), 4.05 (t, J=5.2 Hz, 2H), 3.61 (d, J=6.3 Hz, 4H), 3.36-3.35 (m, 4H), 3.29 (s, 3H), 2.39 (s, 3H).

### Example 247: Synthesis of Compound 511

### Synthesis of Intermediate C415

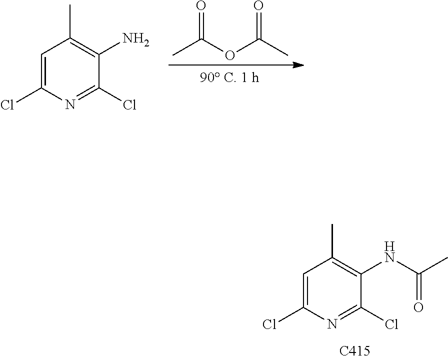

Into a 100 mL 3-necked round-bottom flask were added 2,6-dichloro-4-methylpyridin-3-amine (5.0 g, 28.244 mmol, 1.0 equiv) and acetic anhydride (30 mL) at room temperature. The resulting mixture was stirred for 1 h at 90° C. The resulting mixture was concentrated under reduced pressure. The residue was purified by silica gel column chromatography, eluted with PE/EA (1:1) to afford N-(2,6-dichloro-4-methylpyridin-3-yl) acetamide (2.5 g, 37%) as a solid. LCMS (ES, m/z): 219 [M+H]⁺

### Synthesis of Intermediate C416

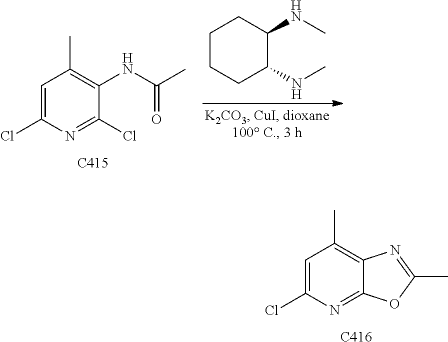

Into a 40 mL vial were added N-(2,6-dichloro-4-methylpyridin-3-yl) acetamide (1.0 g, 4.565 mmol, 1.0 equiv), copper(I) iodide (90.0 mg, 0.457 mmol, 0.1 equiv), (1R,2R)—N1,N2-dimethylcyclohexane-1,2-diamine (130.0 mg, 0.913 mmol, 0.2 equiv), K₂CO₃ (1.3 g, 9.130 mmol, 2.0 equiv) and dioxane (20 mL) at room temperature under nitrogen atmosphere. The resulting mixture was stirred for 3 h at 100° C. under nitrogen atmosphere. The resulting mixture was concentrated under reduced pressure. The residue was purified by silica gel column chromatography, eluted with PE/EA (5:1) to afford 5-chloro-2,7-dimethyl-[1,3] oxazolo[5,4-b]pyridine (0.8 g, 86%) as a solid. LCMS (ES, m/z): 183 [M+H]⁺

### Synthesis of Intermediate C417

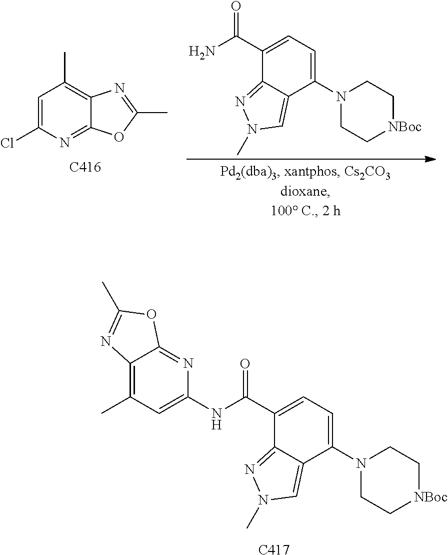

To a solution of 5-chloro-2,7-dimethyl-[1,3] oxazolo[5,4-b]pyridine (110.0 mg, 0.602 mmol, 1.0 equiv) and tert-butyl 4-(7-carbamoyl-2-methylindazol-4-yl)piperazine-1-carboxylate (238.1 mg, 0.662 mmol, 1.1 equiv) in dioxane (3 mL) were added Cs₂CO₃ (393.7 mg, 1.204 mmol, 2.0 equiv) and Pd₂(dba)₃ (55.1 mg, 0.060 mmol, 0.1 equiv). After stirring for 2 h at 100° C. under a nitrogen atmosphere, the resulting mixture was concentrated under reduced pressure. The residue was purified by silica gel column chromatography, eluted with PE/EA (1:3) to afford tert-butyl 4-[7-({2,7-dimethyl-[1,3] oxazolo[5,4-b]pyridin-5-yl}carbamoyl)-2-methylindazol-4-yl]piperazine-1-carboxylate (120 mg, 37%) as a solid. LCMS (ES, m/z): 506 [M+H]⁺

### Synthesis of Compound 511

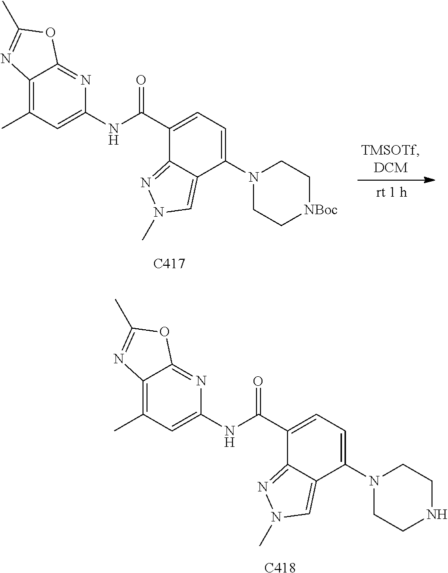

To a stirred solution of tert-butyl 4-[7-({2,7-dimethyl-[1,3]oxazolo[5,4-b]pyridin-5-yl}carbamoyl)-2-methylindazol-4-yl]piperazine-1-carboxylate (70.0 mg, 0.138 mmol, 1.0 equiv) in DCM (2 mL) was added trimethylsilyl triflate (123.0 mg, 0.552 mmol, 4.0 equiv) dropwise at room temperature. The resulting mixture was stirred for 2 h at room temperature. The reaction was monitored by LCMS. The resulting mixture was concentrated under reduced pressure. The crude product was purified by Prep-HPLC (Condition 13, Gradient 1) to afford N-{2,7-dimethyl-[1,3]oxazolo[5,4-b]pyridin-5-yl}-2-methyl-4-(piperazin-1-yl) indazole-7-carboxamide (35 mg, 61%) as a solid. LCMS (ES, m/z): 406 [M+H]^{+ 1}H NMR (400 MHz, DMSO-d₆) δ 11.58 (s, 1H), 8.82 (s, 1H), 8.35 (s, 1H), 8.05 (d, J=8.1 Hz, 1H), 6.51 (d, J=8.2 Hz, 1H), 4.28 (s, 3H), 3.38-3.37 (m, 4H), 2.97-2.90 (m, 4H), 2.62 (s, 3H), 2.57 (s, 3H).

### Example 248: Synthesis of Compound 512

### Synthesis of Intermediate C418

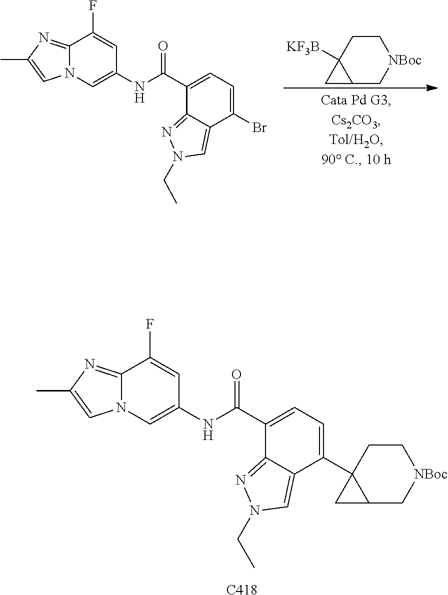

To a stirred mixture of 4-bromo-2-ethyl-N-{8-fluoro-2-methylimidazo[1,2-a]pyridin-6-yl}indazole-7-carboxamide (150 mg, 0.360 mmol, 1 equiv) and tert-butyl 6-(trifluoro-lambda4-boranyl)-3-azabicyclo[4.1.0]heptane-3-carboxylate potassium (120.2 mg, 0.396 mmol, 1.1 equiv) in Toluene (7.5 mL) and H₂O (0.75 mL) were added Cs₂CO₃ (176.1 mg, 0.540 mmol, 1.5 equiv) and Cata Pd G3 (26.2 mg, 0.036 mmol, 0.1 equiv) at room temperature under nitrogen atmosphere. The resulting mixture was stirred for 10 h at 90° C. under nitrogen atmosphere. The resulting mixture was diluted with water (10 mL). The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by silica gel column chromatography, eluted with EA to afford tert-butyl 6-[2-ethyl-7-({8-fluoro-2-methylimidazo[1,2-a]pyridin-6-yl}carbamoyl) indazol-4-yl]-3-azabicyclo[4.1.0]heptane-3-carboxylate (90 mg, 47%) as a solid. LCMS (ES, m/z): 533 [M+H]⁺

### Synthesis of Compound 512

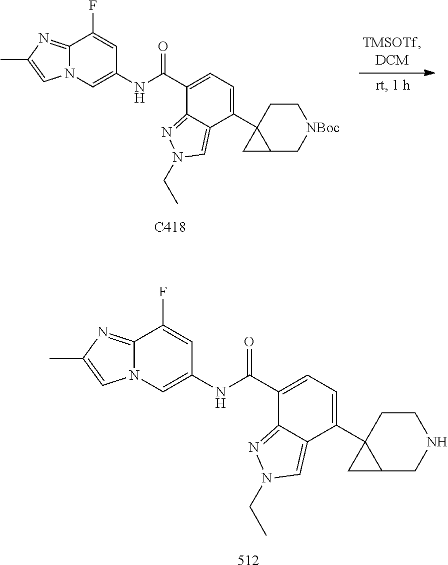

To a stirred mixture of tert-butyl 6-[2-ethyl-7-({8-fluoro-2-methylimidazo[1,2-a]pyridin-6-yl}carbamoyl) indazol-4-yl]-3-azabicyclo[4.1.0]heptane-3-carboxylate (90 mg, 0.169 mmol, 1 equiv) in DCM (2 mL) was added TMSOTf (150.2 mg, 0.676 mmol, 4 equiv) dropwise at room temperature. The resulting mixture was stirred for 1 h at room temperature. The resulting mixture was concentrated under reduced pressure. The crude product was purified by Prep-HPLC (Condition 14, Gradient 2) to afford 4-{3-azabicyclo[4.1.0]heptan-6-yl}-2-ethyl-N-{8-fluoro-2-methylimidazo[1,2-a]pyridin-6-yl}indazole-7-carboxamide 2,2,2-trifluoroacetate (35.4 mg, 48%) as a solid. LCMS (ES, m/z): 433 [M+H]^{+ 1}H NMR (400 MHz, DMSO-d₆) δ 11.28 (s, 1H), 9.42 (d, J=1.6 Hz, 1H), 8.98 (s, 1H), 8.79 (d, J=12.3 Hz, 1H), 8.67-8.66 (m, 1H), 8.14-8.09 (m, 1H), 8.05 (d, J=7.3 Hz, 1H), 7.71 (d, J=11.9 Hz, 1H), 7.23 (d, J=7.3 Hz, 1H), 4.67 (q, J=7.3 Hz, 2H), 3.78 (dd, J=12.6, 7.1 Hz, 1H), 3.18 (dd, J=12.2, 5.7 Hz, 1H), 2.96 (d, J=9.1 Hz, 1H), 2.43 (s, 3H), 2.36 (dt, J=14.4, 4.9 Hz, 1H), 2.20 (ddd, J=14.6, 9.4, 5.5 Hz, 1H), 1.67 (t, J=7.2 Hz, 3H), 1.60 (q, J=6.7 Hz, 1H), 1.28 (t, J=5.5 Hz, 1H), 1.20 (dd, J=9.3, 5.2 Hz, 1H).

### Example 249: Synthesis of Compound 514

### Synthesis of Intermediate C419

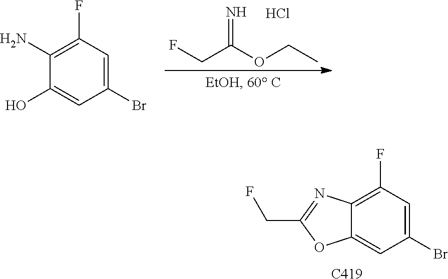

A solution of ethyl 2-fluoroethanimidate hydrochloride (140 mg, 0.989 mmol, 1 equiv) and 2-amino-5-bromo-3-fluorophenol (142.6 mg, 0.692 mmol, 0.7 equiv) in EtOH (4 mL) was stirred for 1 h at 60° C. The mixture was allowed to cool down to room temperature. The resulting mixture was concentrated under reduced pressure. The crude product was used in the next step directly without further purification. LCMS (ES, m/z): 248 [M+H]⁺

### Synthesis of Intermediate C420

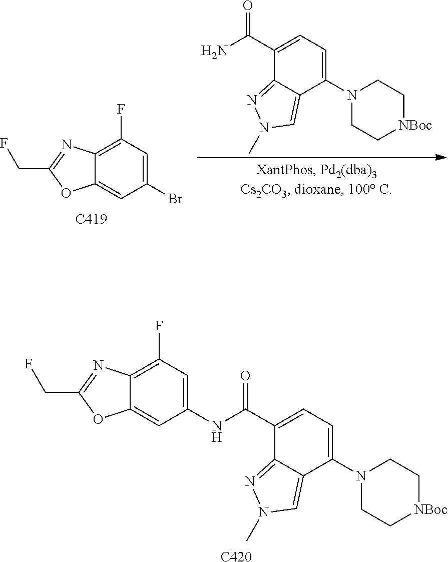

To a solution of tert-butyl 4-(7-carbamoyl-2-methylindazol-4-yl)piperazine-1-carboxylate (100 mg, 0.278 mmol, 1 equiv) and 6-bromo-4-fluoro-2-(fluoromethyl)-1,3-benzoxazole (100 mg, 0.403 mmol, 1.45 equiv) in dioxane (4 mL) were added Cs₂CO₃ (271.9 mg, 0.834 mmol, 3.0 equiv), XantPhos (32.2 mg, 0.056 mmol, 0.2 equiv) and Pd₂(dba)₃ (25.4 mg, 0.028 mmol, 0.1 equiv). After stirring for 3 h at 100° C. under a nitrogen atmosphere, the resulting mixture was concentrated under reduced pressure. The residue was purified by reverse flash chromatography (Condition 5, Gradient 2) to afford tert-butyl 4-(7-{[4-fluoro-2-(fluoromethyl)-1,3-benzoxazol-6-yl]carbamoyl}-2-methylindazol-4-yl)piperazine-1-carboxylate (100 mg, 68%) as a solid. LCMS (ES, m/z): 527[M+H]⁺

### Synthesis of Compound 514

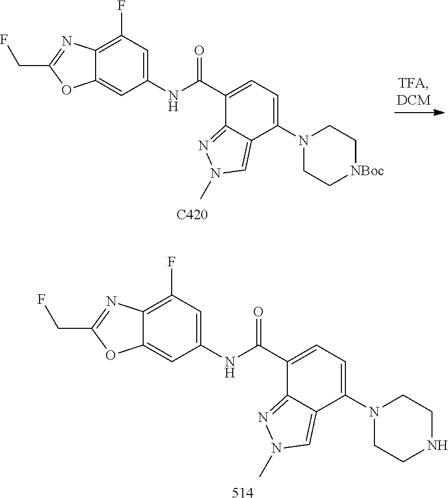

A solution of tert-butyl 4-(7-{[4-fluoro-2-(fluoromethyl)-1,3-benzoxazol-6-yl]carbamoyl}-2-methylindazol-4-yl)piperazine-1-carboxylate (80 mg, 0.152 mmol, 1 equiv) and TFA (0.2 mL) in DCM (2 mL) was stirred for 1 h at room temperature. The mixture was basified to pH 8 with NH₃(g) in MeOH. The resulting mixture was concentrated under reduced pressure. The residue was purified by reverse flash chromatography (Condition 3, Gradient 5) to afford N-[4-fluoro-2-(fluoromethyl)-1,3-benzoxazol-6-yl]-2-methyl-4-(piperazin-1-yl) indazole-7-carboxamide (24 mg, 37.04%) as a solid. LCMS (ES, m/z): 427 [M+H]^{+ 1}H NMR (300 MHz, DMSO-d₆) δ 11.53 (s, 1H), 8.81 (s, 1H), 8.28 (d, J=1.6 Hz, 1H), 8.01 (d, J=8.1 Hz, 1H), 7.73 (dd, J=12.1, 1.7 Hz, 1H), 6.50 (d, J=8.2 Hz, 1H), 5.81 (s, 1H), 5.66 (s, 1H), 4.31 (s, 3H), 3.37 (d, J=5.0 Hz, 4H), 2.92 (t, J=5.1 Hz, 4H).

### Example 250: Synthesis of Compound 515

### Synthesis of Intermediate C421

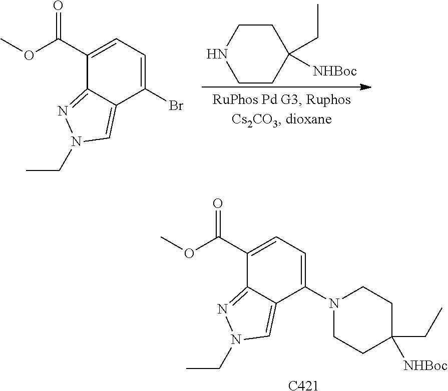 [SMILES: CCn1cc2c(Br)ccc(C(=O)OC)c2n1 ; CCC1(NC(=O)OC(C)(C)C)CCNCC1 ; CCn1cc2c(N3CCC(CC)(NC(=O)OC(C)(C)C)CC3)ccc(C(=O)OC)c2n1 ; "C421"]

To a solution of methyl 4-bromo-2-ethylindazole-7-carboxylate (500 mg, 1.766 mmol, 1 equiv) and tert-butyl N-(4-ethylpiperidin-4-yl)carbamate (604.8 mg, 2.649 mmol, 1.5 equiv) in dioxane (20 mL) were added Cs₂CO₃ (1.73 g, 5.298 mmol, 3.0 equiv), RuPhos (164.8 mg, 0.353 mmol, 0.2 equiv) and RuPhos Palladacycle Gen.3 (147.7 mg, 0.177 mmol, 0.1 equiv). After stirring for 1 h at 80° C. under a nitrogen atmosphere, the mixture was allowed to cool down to room temperature. The resulting mixture was diluted with H₂O (20 mL). The resulting mixture was extracted with EA (3×20 mL). The combined organic layers were washed with brine (1×30 mL), dried over anhydrous Na₂SO₄. After filtration, the filtrate was concentrated under reduced pressure. The residue was purified by reverse flash chromatography (Condition 5, Gradient 2) to afford methyl 4-{4-[(tert-butoxycarbonyl)amino]-4-ethylpiperidin-1-yl}-2-ethylindazole-7-carboxylate (300 mg, 39%) as a solid. LCMS (ES, m/z): 431 [M+H]⁺

### Synthesis of Intermediate C422

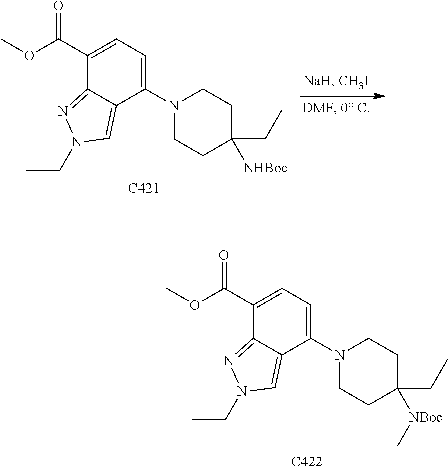

A solution of methyl 4-{4-[(tert-butoxycarbonyl)amino]-4-ethylpiperidin-1-yl}-2-ethylindazole-7-carboxylate (300 mg, 0.697 mmol, 1 equiv) in DMF (4 mL) was treated with NaH (55.7 mg, 1.394 mmol, 2.0 equiv, 60%) for 30 min at 0° C. followed by the addition of CH₃I (148.3 mg, 1.045 mmol, 1.5 equiv) dropwise at 0° C. The resulting mixture was stirred for 2 h at room temperature. The resulting mixture was diluted with H₂O (5 mL). The resulting mixture was extracted with EA (3×10 mL). The combined organic layers were washed with brine (1×5 mL), dried over anhydrous Na₂SO₄. After filtration, the filtrate was concentrated under reduced pressure. The residue was purified by reverse flash chromatography (Condition 5, Gradient 2) to afford methyl 4-{4-[(tert-butoxycarbonyl)(methyl)amino]-4-ethylpiperidin-1-yl}-2-ethylindazole-7-carboxylate (170 mg, 55%) as a solid. LCMS (ES, m/z): 445 [M+H]⁺

### Synthesis of Intermediate C423

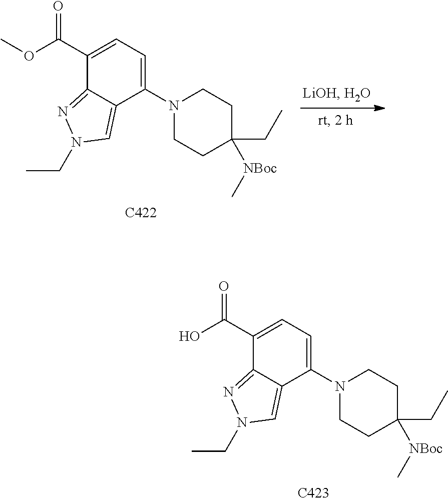

To a solution of methyl 4-{4-[(tert-butoxycarbonyl)(methyl)amino]-4-ethylpiperidin-1-yl}-2-ethylindazole-7-carboxylate (170 mg, 0.382 mmol, 1 equiv) in MeOH (2 mL) and THF (2 mL) was added a solution of LiOH·H₂O (96.2 mg, 2.292 mmol, 6.0 equiv) in H₂O (2 mL) at room temperature. The resulting mixture was stirred for 1 h at room temperature. The mixture was acidified to pH 2 with 2 M HCl. The resulting mixture was extracted with EA (3×5 mL). The combined organic layers were washed with brine (1×5 mL), dried over anhydrous Na₂SO₄. After filtration, the filtrate was concentrated under reduced pressure. The residue was purified by reverse flash chromatography (Condition 5, Gradient 6) to afford 4-{4-[(tert-butoxycarbonyl)(methyl)amino]-4-ethylpiperidin-1-yl}-2-ethylindazole-7-carboxylic acid (141 mg, 85%) as a solid. LCMS (ES, m/z): 431 [M+H]⁺

### Synthesis of Intermediate C424

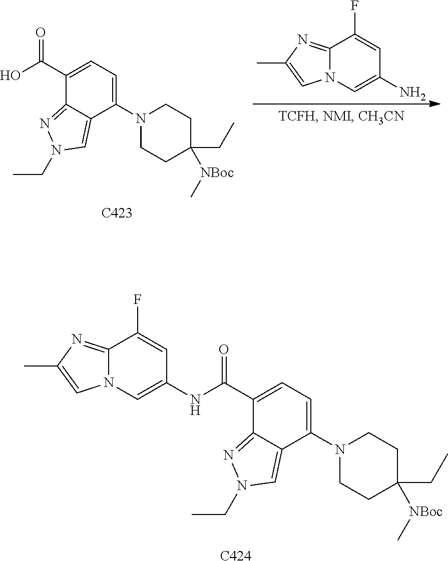

To a stirred solution of 4-{4-[(tert-butoxycarbonyl)(methyl)amino]-4-ethylpiperidin-1-yl}-2-ethylindazole-7-carboxylic acid (140 mg, 0.325 mmol, 1 equiv) and TCFH (118.6 mg, 0.423 mmol, 1.3 equiv) in CH₃CN (3 mL) were added NMI (93.4 mg, 1.137 mmol, 3.5 equiv) and 8-fluoro-2-methylimidazo[1,2-a]pyridin-6-amine (64.4 mg, 0.390 mmol, 1.2 equiv) in portions at room temperature. The resulting mixture was stirred for 2 h at room temperature. The precipitated solids were collected by filtration and washed with H₂O (1×5 mL). The crude product was used in the next step directly without further purification. LCMS (ES, m/z): 578 [M+H]⁺

### Synthesis of Compound 515

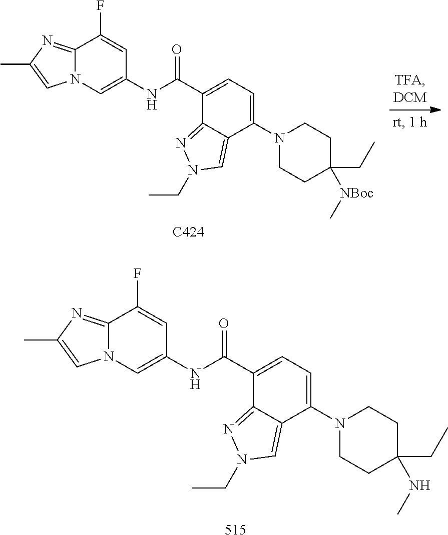

A solution of tert-butyl N-{4-ethyl-1-[2-ethyl-7-({8-fluoro-2-methylimidazo[1,2-a]pyridin-6-yl}carbamoyl) indazol-4-yl]piperidin-4-yl}-N-methylcarbamate (70 mg, 0.121 mmol, 1 equiv) and TFA (0.2 mL) in DCM (2 mL) was stirred for 1 h at room temperature. The mixture was basified to pH 8 with NH₃(g) in MeOH. The resulting mixture was concentrated under reduced pressure. The residue was purified by reverse flash chromatography (Condition 3, Gradient 2) to afford 2-ethyl-4-[4-ethyl-4-(methylamino)piperidin-1-yl]-N-{8-fluoro-2-methylimidazo[1,2-a]pyridin-6-yl}indazole-7-carboxamide (28 mg, 48%) as a solid. LCMS (ES, m/z): 478 [M+H]^{+ 1}H NMR (300 MHz, DMSO-d₆) δ 11.10 (s, 1H), 9.21 (d, J=1.6 Hz, 1H), 8.80 (s, 1H), 7.96 (d, J=8.1 Hz, 1H), 7.90 (dd, J=3.2, 1.0 Hz, 1H), 7.29 (dd, J=12.3, 1.7 Hz, 1H), 6.46 (d, J=8.3 Hz, 1H), 4.59 (q, J=7.3 Hz, 2H), 3.58 (d, J=12.5 Hz, 2H), 3.49-3.30 (m, 2H), 2.38-2.32 (m, 3H), 2.16 (s, 3H), 1.72-1.48 (m, 7H), 1.42 (q, J=7.4 Hz, 2H), 0.80 (t, J=7.4 Hz, 3H).

### Example 251: Synthesis of Compounds 516 and 517

### Synthesis of Intermediate C425

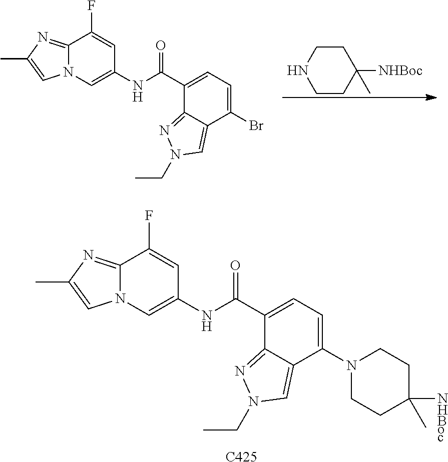

To a stirred solution of 4-bromo-2-ethyl-N-{8-fluoro-2-methylimidazo[1,2-a]pyridin-6-yl}indazole-7-carboxamide (300 mg, 0.721 mmol, 1 equiv), tert-butyl N-(4-methylpiperidin-4-yl)carbamate (154 mg, 0.721 mmol, 1 equiv) and Cs₂CO₃ (470 mg, 1.442 mmol, 2.0 equiv) in dioxane (5 mL) were added RuPhos (67.26 mg, 0.144 mmol, 0.2 equiv) and RuPhos Palladacycle Gen.3 (60.28 mg, 0.072 mmol, 0.1 equiv) at room temperature under nitrogen atmosphere. The resulting mixture was stirred for 3 h at 100° C. The mixture was allowed to cool down to room temperature. The resulting mixture was concentrated under reduced pressure. The residue was purified by silica gel column chromatography, eluted with EA/MeOH (20:1) to afford tert-butyl N-{1-[2-ethyl-7-({8-fluoro-2-methylimidazo[1,2-a]pyridin-6-yl}carbamoyl) indazol-4-yl]-4-methylpiperidin-4-yl}carbamate (260 mg, 66%) as a solid. LCMS (ES, m/z): 550 [M+H]⁺

### Synthesis of Intermediate C426

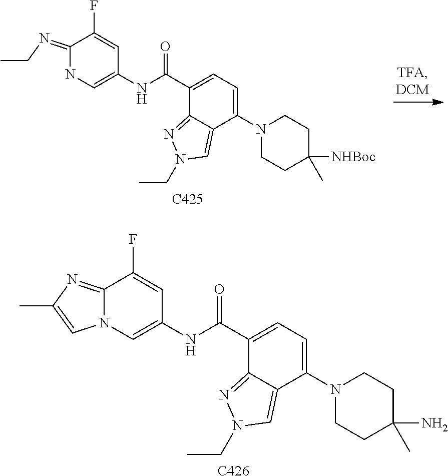

A solution of tert-butyl N-{1-[2-ethyl-7-({8-fluoro-2-methylimidazo[1,2-a]pyridin-6-yl}carbamoyl) indazol-4-yl]-4-methylpiperidin-4-yl}carbamate (250 mg, 0.455 mmol, 1 equiv) and trifluoroacetic acid (3 mL) in DCM (6 mL) was stirred for 2 h at room temperature under nitrogen atmosphere. The resulting mixture was concentrated under vacuum. The residue was purified by reverse flash chromatography (Condition 3, Gradient 9) to afford 4-(4-amino-4-methylpiperidin-1-yl)-2-ethyl-N-{8-fluoro-2-methylimidazo[1,2-a]pyridin-6-yl}indazole-7-carboxamide (130 mg, 64%) as a solid. LCMS (ES, m/z): 450 [M+H]⁺

### Synthesis of Compounds 516 and 517

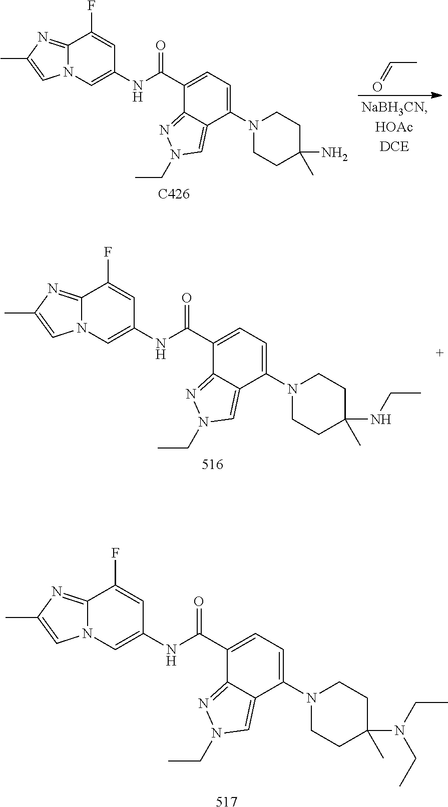

To a stirred solution of 4-(4-amino-4-methylpiperidin-1-yl)-2-ethyl-N-{8-fluoro-2-methylimidazo[1,2-a]pyridin-6-yl}indazole-7-carboxamide (150 mg, 0.334 mmol, 1 equiv) and acetaldehyde (73.50 mg, 1.670 mmol, 5 equiv) in DCE (5 mL) were added HOAc (60.11 mg, 1.002 mmol, 3 equiv) and NaBH₃CN (104.84 mg, 1.670 mmol, 5 equiv) portions at room temperature. The resulting mixture was stirred for 1 h at room temperature. The reaction was quenched by the addition of water (2 mL) at room temperature. The resulting mixture was concentrated under vacuum. The residue was purified by reverse flash chromatography (Condition 3, Gradient 3) to afford 2-ethyl-4-[4-(ethylamino)-4-methylpiperidin-1-yl]-N-{8-fluoro-2-methylimidazo[1,2-a]pyridin-6-yl}indazole-7-carboxamide (3 mg, 2%) as a solid and 4-[4-(diethylamino)-4-methylpiperidin-1-yl]-2-ethyl-N-{8-fluoro-2-methylimidazo[1,2-a]pyridin-6-yl}indazole-7-carboxamide trifluoroacetic acid (25 mg, 12%) as a solid which was further purified by reverse flash chromatography (Condition 3, Gradient 3). Compound 516: LCMS (ES, m/z): 408 [M+H]^{+ 1}H NMR (300 MHz, Methanol-d₄) δ 9.11 (s, 1H), 8.55 (s, 1H), 8.10 (d, J=8.2 Hz, 1H), 7.74 (s, 1H), 7.25 (d, J=12.1 Hz, 1H), 6.56 (d, J=8.1 Hz, 1H), 4.63 (q, J=7.1 Hz, 2H), 3.81 (d, J=12.9 Hz, 2H), 3.20 (d, J=12.7 Hz, 2H), 2.80 (d, J=5.9 Hz, 2H), 2.44 (s, 3H), 1.97-1.83 (m, 4H), 1.72 (t, J=7.3 Hz, 3H), 1.33 (s, 3H), 1.22 (t, J=7.1 Hz, 3H). Compound 517: LCMS (ES, m/z): 506 [M+H]^{+ 1}H NMR (300 MHz, DMSO-d₆) δ 11.27 (s, 1H), 9.44 (s, 1H), 8.92 (s, 1H), 8.30 (s, 1H), 8.13 (s, 1H), 8.03 (d, J=8.1 Hz, 1H), 7.76 (d, J=12.0 Hz, 1H), 6.58 (d, J=8.2 Hz, 1H), 4.63 (q, J=7.2 Hz, 2H), 4.03 (d, J=13.2 Hz, 2H), 3.49 (dd, J=13.3, 7.0 Hz, 2H), 3.20 (d, J=12.7 Hz, 2H), 3.06 (dt, J=13.5, 6.7 Hz, 2H), 2.44 (s, 3H), 2.09 (s, 4H), 1.64 (t, J=7.3 Hz, 3H), 1.45 (s, 3H), 1.31 (t, J=7.2 Hz, 6H).

### Example 252: Synthesis of Compound 518

### Synthesis of Intermediate C427

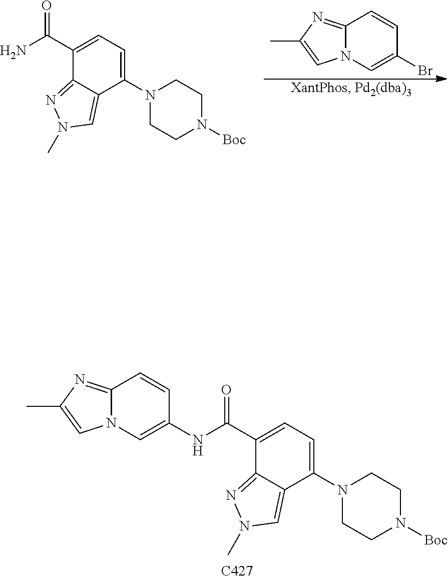

To a solution of 6-bromo-2-methylimidazo[1,2-a]pyridine (100 mg, 0.474 mmol, 1 equiv) and tert-butyl 4-(7-carbamoyl-2-methylindazol-4-yl)piperazine-1-carboxylate (170.30 mg, 0.474 mmol, 1.0 equiv) in 1,4-dioxane (5 mL) were added Cs₂CO₃ (308.74 mg, 0.948 mmol, 2.0 equiv), XantPhos (54.83 mg, 0.095 mmol, 0.2 equiv) and Pd₂(dba)₃ (43.39 mg, 0.047 mmol, 0.1 equiv). After stirring for 5 h at 90° C. under a nitrogen atmosphere, the resulting mixture was concentrated under reduced pressure. The residue was purified by silica gel column chromatography, eluted with PE/EA (1:10) to afford tert-butyl 4-[2-methyl-7-({2-methylimidazo[1,2-a]pyridin-6-yl}carbamoyl) indazol-4-yl]piperazine-1-carboxylate (100 mg, 43%) as a solid. LCMS (ES, m/z): 490 [M+H]⁺

### Synthesis of Compound 518

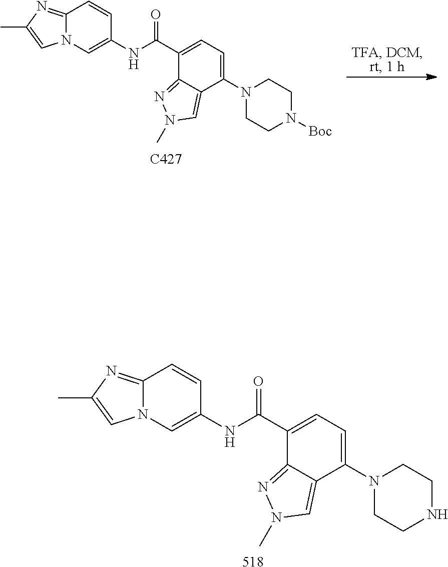

A solution of tert-butyl 4-[2-methyl-7-({2-methylimidazo[1,2-a]pyridin-6-yl}carbamoyl) indazol-4-yl]piperazine-1-carboxylate (100 mg, 0.204 mmol, 1 equiv) and TFA (3 mL) in DCM (3 mL) was stirred for 1 h at room temperature. The resulting mixture was concentrated under reduced pressure. The residue was basified to pH 8 with 7M NH₃(g) in MeOH. The resulting mixture was concentrated under vacuum. The residue was purified by reverse flash chromatography (Condition 3, Gradient 10) to afford 2-methyl-N-{2-methylimidazo[1,2-a]pyridin-6-yl}-4-(piperazin-1-yl) indazole-7-carboxamide (30 mg, 37.71%) as a solid. LCMS (ES, m/z): 390 [M+H]^{+ 1}H NMR (300 MHz, DMSO-d₆) δ 11.07 (s, 1H), 9.35 (dd, J=2.1, 0.9 Hz, 1H), 8.80 (s, 1H), 7.99 (d, J=8.1 Hz, 1H), 7.76 (s, 1H), 7.48 (d, J=9.5 Hz, 1H), 7.25 (dd, J=9.5, 2.0 Hz, 1H), 6.49 (d, J=8.2 Hz, 1H), 4.29 (s, 3H), 3.35 (d, J=5.3 Hz, 4H), 2.93 (dd, J=6.0, 3.6 Hz, 4H), 2.33 (d, J=0.9 Hz, 3H).

### Example 253: Synthesis of Compound 520

### Synthesis of Intermediate C428

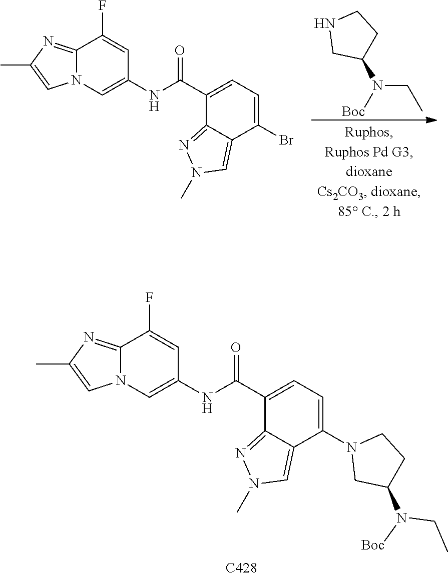 [SMILES: Cc1cn2cc(NC(=O)c3ccc(Br)c4cn(C)nc34)cc(F)c2n1 ; CCN(C(=O)OC(C)(C)C)[C@@H]1CCNC1 ; CCN(C(=O)OC(C)(C)C)[C@@H]1CCN(c2ccc(C(=O)Nc3cc(F)c4nc(C)cn4c3)c3nn(C)cc23)C1 ; "C428"]

To a solution of 4-bromo-N-{8-fluoro-2-methylimidazo[1,2-a]pyridin-6-yl}-2-methylindazole-7-carboxamide (120.0 mg, 0.298 mmol, 1.0 equiv) and tert-butyl N-ethyl-N-[(3R)-pyrrolidin-3-yl]carbamate (83.1 mg, 0.387 mmol, 1.3 equiv) in dioxane (1 mL) were added Cs₂CO₃ (194.4 mg, 0.596 mmol, 2.0 equiv) 184 and Ruphos (27.8 mg, 0.060 mmol, 0.2 equiv), RuPhos Palladacycle Gen.3 (24.9 mg, 0.030 mmol, 0.1 equiv). After stirring for 2 h at 85° C. under a nitrogen atmosphere, the resulting mixture was concentrated under reduced pressure. The residue was purified by silica gel column chromatography, eluted with CH₂Cl₂/MeOH (20:1) to afford tert-butyl N-ethyl-N-[(3R)-1-[7-({8-fluoro-2-methylimidazo[1,2-a]pyridin-6-yl}carbamoyl)-2-methylindazol-4-yl]pyrrolidin-3-yl]carbamate (130 mg, 80%) as a solid. LCMS (ES, m/z): 536 [M+H]⁺

### Synthesis of Compound 520

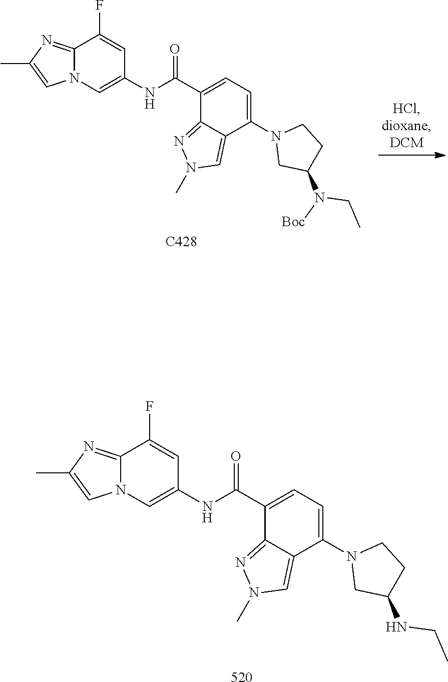

Into a 40 mL vial were added tert-butyl N-ethyl-N-[(3R)-1-[7-({8-fluoro-2-methylimidazo[1,2-a]pyridin-6-yl}carbamoyl)-2-methylindazol-4-yl]pyrrolidin-3-yl]carbamate (120.0 mg, 0.224 mmol, 1.0 equiv), DCM (2 mL) and HCl(gas) in 1,4-dioxane (1 mL) at room temperature. The resulting mixture was stirred for 1 h at room temperature. The reaction was monitored by LCMS. The resulting mixture was concentrated under reduced pressure. The crude product was purified by Prep-HPLC (Condition 13, Gradient 1) to afford 4-[(3R)-3-(ethylamino)pyrrolidin-1-yl]-N-{8-fluoro-2-methylimidazo[1,2-a]pyridin-6-yl}-2-methylindazole-7-carboxamide (25 mg, 25.34%) as a solid. LCMS (ES, m/z): 436 [M+H]⁺ 1H NMR (400 MHz, DMSO-d₆) δ 11.01 (s, 1H), 9.19 (d, J=1.6 Hz, 1H), 8.83 (s, 1H), 7.96-7.86 (m, 2H), 7.30 (dd, J=12.4, 1.7 Hz, 1H), 6.01 (d, J=8.4 Hz, 1H), 4.27 (s, 3H), 3.84-3.70 (m, 2H), 3.63 (d, J=8.4 Hz, 1H), 3.45 (dd, J=10.3, 5.1 Hz, 2H), 2.63 (q, J=7.1 Hz, 2H), 2.35 (s, 3H), 2.17 (dq, J=12.8, 6.6 Hz, 1H), 1.91 (dt, J=12.2, 6.3 Hz, 2H), 1.05 (t, J=7.1 Hz, 3H).

### Example 254: Synthesis of Compound 522

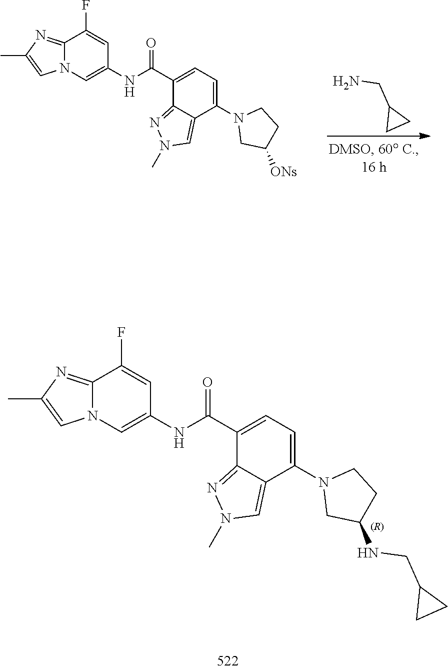

Into a 8 mL vial were added (3S)-1-[7-({8-fluoro-2-methylimidazo[1,2-a]pyridin-6-yl}carbamoyl)-2-methylindazol-4-yl]pyrrolidin-3-yl 4-nitrobenzenesulfonate (100.0 mg, 0.168 mmol, 1.0 equiv), DMSO (0.5 mL) and 1-cyclopropylmethanamine (47.9 mg, 0.672 mmol, 4.0 equiv) at room temperature. The resulting mixture was stirred for overnight at 60° C. The resulting mixture was diluted with water (3 mL). The resulting mixture was extracted with CH₂Cl₂ (2×5 mL). The combined organic layers were washed with brine (1×3 mL), dried over anhydrous Na₂SO₄. After filtration, the filtrate was concentrated under reduced pressure. The residue was purified by Prep-TLC (THF) to afford 4-[(3R)-3-[(cyclopropylmethyl)amino]pyrrolidin-1-yl]-N-{8-fluoro-2-methylimidazo[1,2-a]pyridin-6-yl}-2-methylindazole-7-carboxamide (50 mg, 25%) as a solid. The crude product (50 mg) was purified by Chiral Prep-HPLC w (Condition 8, Gradient 1) to afford 4-[(3R)-3-[(cyclopropylmethyl)amino]pyrrolidin-1-yl]-N-{8-fluoro-2-methylimidazo[1,2-a]pyridin-6-yl}-2-methylindazole-7-carboxamide (20 mg, 25%) as a solid. LCMS (ES, m/z): 462 [M+H]^{+ 1}H NMR (400 MHz, DMSO-d₆) δ 11.02 (s, 1H), 9.20 (d, J=1.7 Hz, 1H), 8.83 (s, 1H), 7.93 (d, J=8.3 Hz, 1H), 7.89 (d, J=3.1 Hz, 1H), 7.31 (dd, J=12.5, 1.7 Hz, 1H), 6.02 (d, J=8.4 Hz, 1H), 4.27 (s, 3H), 3.84-3.71 (m, 2H), 3.64 (s, 1H), 3.50 (d, J=30.5 Hz, 2H), 2.35 (s, 3H), 2.18 (s, 1H), 1.95 (s, 1H), 1.24 (s, 1H), 0.89 (d, J=22.6 Hz, 1H), 0.43 (d, J=7.7 Hz, 2H), 0.16 (d, J=4.6 Hz, 2H).

### Example 255: Synthesis of Compound 524

### Synthesis of Intermediate C429

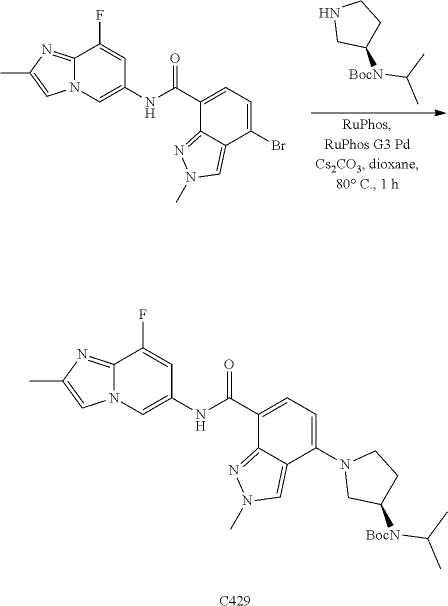 [SMILES: Cc1cn2cc(NC(=O)c3ccc(Br)c4cn(C)nc34)cc(F)c2n1 ; CC(C)N(C(=O)OC(C)(C)C)[C@@H]1CCNC1 ; Cc1cn2cc(NC(=O)c3ccc(N4CC[C@@H](N(C(=O)OC(C)(C)C)C(C)C)C4)c4cn(C)nc34)cc(F)c2n1 ; "C429"]

A solution of 4-bromo-N-{8-fluoro-2-methylimidazo[1,2-a]pyridin-6-yl}-2-methylindazole-7-carboxamide (100 mg, 0.249 mmol, 1 equiv) in dioxane (1 mL) was added tert-butyl N-isopropyl-N-[(3R)-pyrrolidin-3-yl] carbamate (85.1 mg, 0.373 mmol, 1.5 equiv), Cs₂CO₃ (202.5 mg, 0.623 mmol, 2.5 equiv) and Ruphos (23.3 mg, 0.050 mmol, 0.2 equiv), RuPhos Palladacycle Gen.3 (20.8 mg, 0.025 mmol, 0.1 equiv) under nitrogen atmosphere. The reaction was stirred for 1 h at 80° C. to give a black solution. The resulting mixture was concentrated under vacuum. The residue was purified by silica gel column chromatography, eluted with PE/EA (60%) to afford tert-butyl N-[(3R)-1-[7-({8-fluoro-2-methylimidazo[1,2-a]pyridin-6-yl}carbamoyl)-2-methylindazol-4-yl] pyrrolidin-3-yl]-N-isopropylcarbamate (135 mg, 99%) as an oil. LCMS (ES, m/z): 550 [M+H]⁺

### Synthesis of Compound 524

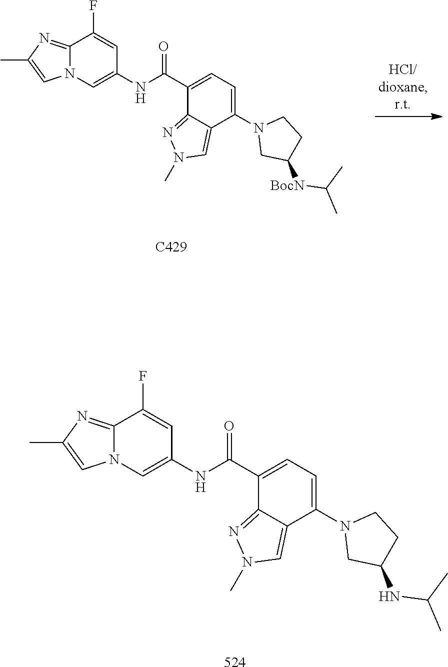

A solution of tert-butyl N-[(3R)-1-[7-({8-fluoro-2-methylimidazo[1,2-a] pyridin-6-yl}carbamoyl)-2-methylindazol-4-yl] pyrrolidin-3-yl]-N-isopropylcarbamate (150 mg, 0.273 mmol, 1 equiv) in DCM (2 mL) was added HCl(gas) in 1,4-dioxane (1 mL, 4M). The mixture was stirred for 1 h at 20° C. The reaction was monitored by LCMS. The resulting mixture was concentrated under vacuum. The crude product was purified by Prep-HPLC (Condition 10, Gradient 3) to afford N-{8-fluoro-2-methylimidazo[1,2-a] pyridin-6-yl}-4-[(3R)-3-(isopropylamino) pyrrolidin-1-yl]-2-methylindazole-7-carboxamide (60 mg, 49%) as a solid. LCMS (ES, m/z): 450 [M+H]^{+ 1}H NMR (400 MHz, DMSO-d₆) δ 11.01 (s, 1H), 9.20 (d, J=1.6 Hz, 1H), 8.83 (s, 1H), 7.93 (d, J=8.3 Hz, 1H), 7.88 (s, 1H), 7.31 (dd, J=12.4, 1.7 Hz, 1H), 6.02 (d, J=8.4 Hz, 1H), 4.27 (s, 3H), 3.83 (t, J=8.4 Hz, 1H), 3.74 (m, 1H), 3.69-3.51 (m, 2H), 3.37 (d, J=13.2 Hz, 2H), 2.89 (p, J=6.2 Hz, 1H), 2.35 (s, 3H), 2.18 (dt, J=12.6, 6.2 Hz, 1H), 1.87 (dt, J=12.5, 6.9 Hz, 1H), 1.03 (t, J=5.8 Hz, 6H).

### Example 256: Synthesis of Compound 525

### Synthesis of Intermediate C430

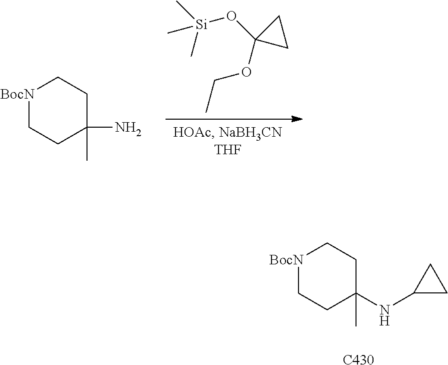

To a stirred solution of tert-butyl 4-amino-4-methylpiperidine-1-carboxylate (1 g, 4.666 mmol, 1 equiv) and (1-ethoxycyclopropoxy) trimethylsilane (1.63 g, 9.332 mmol, 2 equiv) in tetrahydrofuran (20 mL) were added HOAc (0.84 g, 13.998 mmol, 3 equiv) and NaBH₃CN (0.88 g, 13.998 mmol, 3 equiv) at room temperature. The resulting mixture was stirred for overnight at 60° C. The reaction was quenched by the addition of water (20 mL) at room temperature. The resulting mixture was extracted with EtOAc (3×30 mL). The combined organic layers were washed with brine (2×10 mL), dried over anhydrous Na₂SO₄. After filtration, the filtrate was concentrated under reduced pressure. The residue was purified by reverse flash chromatography (Condition 3, Gradient 3) to afford tert-butyl 4-(cyclopropylamino)-4-methylpiperidine-1-carboxylate (260 mg, 22%) as an oil. LCMS (ES, m/z): 255 [M+H]⁺

### Synthesis of Intermediate C431

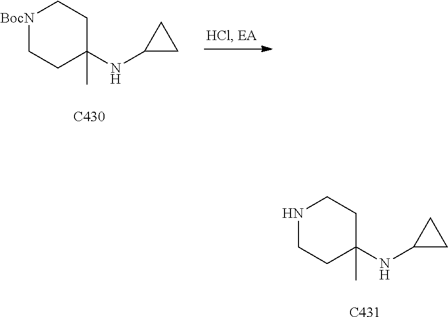

Into a 40 mL vial were added tert-butyl 4-(cyclopropylamino)-4-methylpiperidine-1-carboxylate (260 mg, 1.022 mmol, 1 equiv) and HCl(gas) in 1,4-dioxane (5 mL) at room temperature. The resulting mixture was stirred for 1 h at room temperature. The resulting mixture was concentrated under vacuum. The crude product was used in the next step directly without further purification. LCMS (ES, m/z): 155 [M+H]⁺

### Synthesis of Compound 525

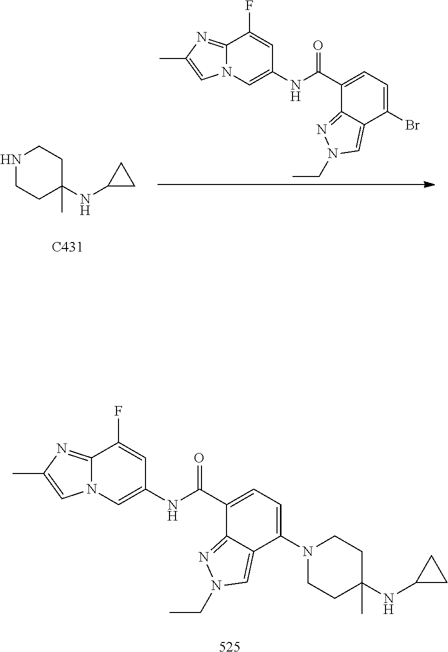

To a stirred solution of N-cyclopropyl-4-methylpiperidin-4-amine hydrochloride (100 mg, 0.524 mmol, 1 equiv) and 4-bromo-2-ethyl-N-{8-fluoro-2-methylimidazo[1,2-a]pyridin-6-yl}indazole-7-carboxamide (327 mg, 0.786 mmol, 1.5 equiv) in 1,4-dioxane (10 mL) were added Cs₂CO₃ (512 mg, 1.572 mmol, 3 equiv) and Pd-PEPPSI-IPentCl 2-methylpyridine (o-picoline (22 mg, 0.026 mmol, 0.05 equiv) at room temperature under nitrogen atmosphere. The resulting mixture was stirred for 48 h at 100° C. The mixture was allowed to cool down to room temperature. The reaction was quenched by the addition of water (20 mL) at room temperature. The resulting mixture was extracted with EtOAc (2×30 mL). The combined organic layers were washed with brine (3×5 mL), dried over anhydrous Na₂SO₄. After filtration, the filtrate was concentrated under reduced pressure. The residue was purified by reverse flash chromatography (Condition 3, Gradient 3) to afford 4-[4-(cyclopropylamino)-4-methylpiperidin-1-yl]-2-ethyl-N-{8-fluoro-2-methylimidazo[1,2-a]pyridin-6-yl}indazole-7-carboxamide (19 mg, 7%) as a solid. LCMS (ES, m/z): 490 [M+H]^{+ 1}H NMR (300 MHz, DMSO-d₆) δ 11.11 (s, 1H), 9.21 (s, 1H), 8.80 (s, 1H), 8.01-7.88 (m, 2H), 7.30 (d, J=12.4 Hz, 1H), 6.48 (d, J=8.7 Hz, 1H), 4.59 (d, J=7.4 Hz, 2H), 3.36-3.29 (m, 4H), 2.49 (s, 1H), 2.36 (d, J=2.6 Hz, 3H), 2.11 (s, 1H), 1.81 (d, J=13.2 Hz, 2H), 1.63 (dt, J=9.0, 4.5 Hz, 5H), 1.21 (d, J=2.6 Hz, 3H), 0.42 (d, J=3.6 Hz, 2H), 0.24 (s, 2H).

### Example 256: Synthesis of Compound 526

### Synthesis of Intermediate C432

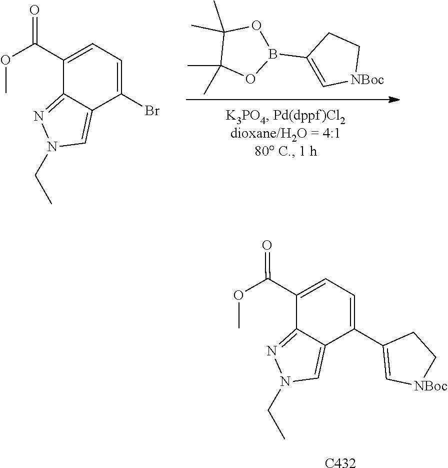 [SMILES: CCn1cc2c(Br)ccc(C(=O)OC)c2n1 ; CC(C)(C)OC(=O)N1C=C(B2OC(C)(C)C(C)(C)O2)CC1 ; CCn1cc2c(C3=CN(C(=O)OC(C)(C)C)CC3)ccc(C(=O)OC)c2n1 ; "C432"]

A solution of methyl 4-bromo-2-ethylindazole-7-carboxylate (500 mg, 1.766 mmol, 1.00 equiv) in dioxane/H₂O (4:1) (1 mL) were added tert-butyl 3-(4,4,5,5-tetramethyl-1,3,2-dioxaborolan-2-yl)-4,5-dihydropyrrole-1-carboxylate (781.96 mg, 2.649 mmol, 1.5 equiv), K₃PO₄ (749.7 mg, 3.532 mmol, 2.0 equiv) and Pd(dppf)Cl₂ (129.2 mg, 0.177 mmol, 0.1 equiv) under nitrogen atmosphere. The mixture was stirred at 80° C. for 1 h. The resulting mixture was concentrated under vacuum to give the crude product. The residue was purified by silica gel column chromatography, eluted with PE/EA (65%) to afford methyl 4-[1-(tert-butoxycarbonyl)-4,5-dihydropyrrol-3-yl]-2-ethylindazole-7-carboxylate (500 mg, 76%) as an oil. LCMS (ES, m/z): 372 [M+H]⁺

### Synthesis of Intermediate C433

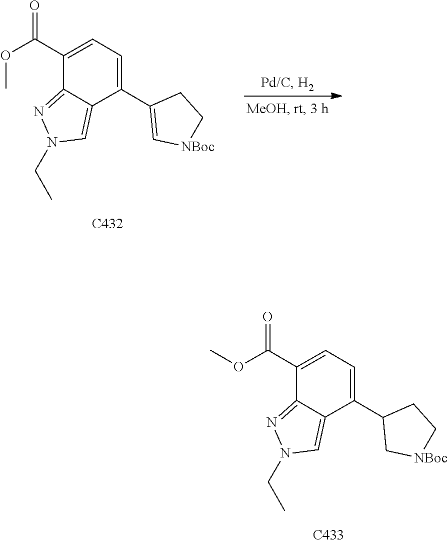

A solution of methyl 4-[1-(tert-butoxycarbonyl)-4,5-dihydropyrrol-3-yl]-2-ethylindazole-7-carboxylate (500 mg, 1.346 mmol, 1 equiv) in MeOH (15 mL) was added Pd/C (200 mg, 10% w/w). The mixture was stirred for 3 h at room temperature under hydrogen atmosphere. The resulting mixture was concentrated under vacuum. The residue was purified by silica gel column chromatography, eluted with PE/EA (1/1) to afford methyl 4-[1-(tert-butoxycarbonyl) pyrrolidin-3-yl]-2-ethylindazole-7-carboxylate (270 mg, 54%) as an oil. LCMS (ES, m/z): 374 [M+H]⁺

### Synthesis of Intermediate C434

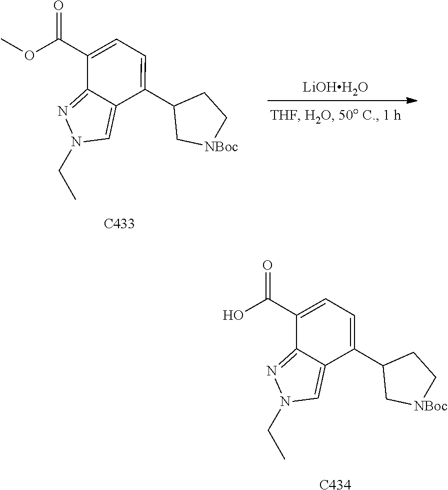

A solution of methyl 4-[1-(tert-butoxycarbonyl) pyrrolidin-3-yl]-2-ethylindazole-7-carboxylate (270 mg, 0.723 mmol, 1 equiv) in THF/MeOH/H₂O (1:1:1) (6 mL) was added LiOH·H₂O (303.36 mg, 7.230 mmol, 10.0 equiv). The mixture was stirred for 1 h at 50° C. to give a yellow mixture. The reaction was monitored by LCMS. The resulting mixture was concentrated under vacuum and acidified to pH 5-6 with 1M HCl. The resulting mixture was extracted with EtOAc (3×30 mL), dried over anhydrous Na₂SO₄. After filtration, the filtrate was concentrated under reduced pressure to afford 4-[1-(tert-butoxycarbonyl) pyrrolidin-3-yl]-2-ethylindazole-7-carboxylic acid (170 mg, 65%) as a oil. LCMS (ES, m/z): 360 [M+H]⁺

### Synthesis of Intermediate C435

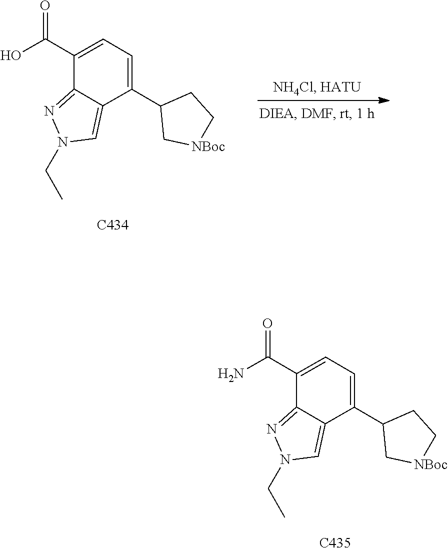 [SMILES: CCn1cc2c(C3CCN(C(=O)OC(C)(C)C)C3)ccc(C(=O)O)c2n1 ; "C434" ; CCn1cc2c(C3CCN(C(=O)OC(C)(C)C)C3)ccc(C(N)=O)c2n1 ; "C435"]

A solution of 4-[1-(tert-butoxycarbonyl) pyrrolidin-3-yl]-2-ethylindazole-7-carboxylic acid (170 mg, 0.473 mmol, 1 equiv) in DMF (1.5 mL) was added NH₄Cl (126.5 mg, 2.365 mmol, 5 equiv), HATU (539.5 mg, 1.419 mmol, 3 equiv) and DIEA (91.7 mg, 0.710 mmol, 1.5 equiv). The mixture was stirred at room temperature for 1 h. The resulting mixture was washed with 2×10 mL of water. Then extracted with EtOAc (3×20 mL), and dried over anhydrous Na₂SO₄. After filtration, the filtrate was concentrated under reduced pressure. The residue was purified by silica gel column chromatography, eluted with PE in EA (55%) to afford tert-butyl 3-(7-carbamoyl-2-ethylindazol-4-yl) pyrrolidine-1-carboxylate (125 mg, 74%) as an oil. LCMS (ES, m/z): 359 [M+H]⁺

### Synthesis of Intermediate C436

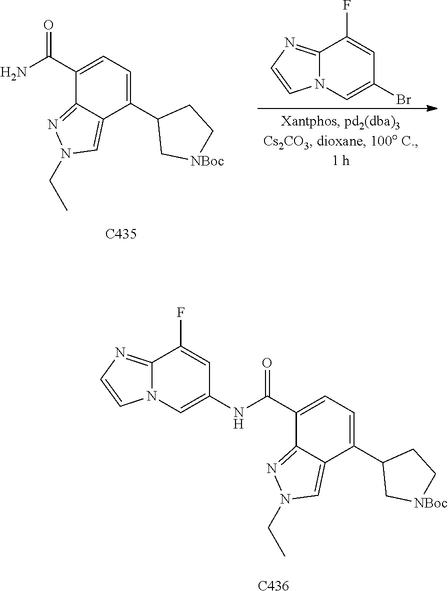

A solution of tert-butyl 3-(7-carbamoyl-2-ethylindazol-4-yl) pyrrolidine-1-carboxylate (125 mg, 0.349 mmol, 1 equiv) in dioxane (1.5 mL) was added 6-bromo-8-fluoroimidazo[1,2-a]pyridine (112.4 mg, 0.523 mmol, 1.5 equiv), Cs₂CO₃ (227.2 mg, 0.698 mmol, 2 equiv), XantPhos (40.3 mg, 0.070 mmol, 0.2 equiv) and Pd₂(dba)₃ (31.9 mg, 0.035 mmol, 0.1 equiv) under nitrogen atmosphere. The mixture was stirred at 100° C. for 1 h to give a black solution. The reaction was monitored by LCMS. The resulting mixture was concentrated under vacuum. The residue was purified by silica gel column chromatography, eluted with PE/EA (40%) to afford tert-butyl 3-[2-ethyl-7-({8-fluoroimidazo[1,2-a] pyridin-6-yl} carbamoyl) indazol-4-yl]pyrrolidine-1-carboxylate (50 mg, 29%) as an oil. LCMS (ES, m/z): 493 [M+H]⁺

### Synthesis of Compound 526

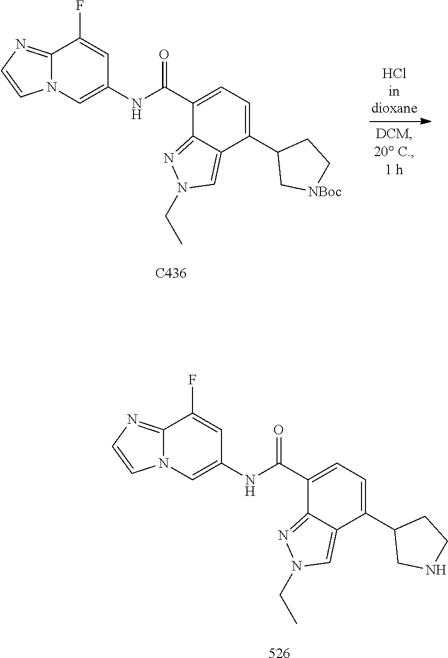

A solution of tert-butyl 3-[2-ethyl-7-({8-fluoroimidazo[1,2-a] pyridin-6-yl} carbamoyl) indazol-4-yl] pyrrolidine-1-carboxylate (50 mg, 0.102 mmol, 1 equiv) in DCM (1 mL) was added HCl(gas) in 1,4-dioxane (1 mL, 4M). The mixture was stirred for 1 h at 20° C. The reaction was monitored by LCMS. The resulting mixture was concentrated under vacuum. The crude product was purified by Prep-HPLC (Condition 10, Gradient 2) to afford 2-ethyl-N-{8-fluoroimidazo[1,2-a] pyridin-6-yl}-4-(pyrrolidin-3-yl) indazole-7-carboxamide (6.8 mg, 17%) as a solid. LCMS (ES, m/z): 393 [M+H]^{+ 1}H NMR (400 MHz, Methanol-d₄) δ 9.29 (d, J=1.8 Hz, 1H), 8.66 (d, J=1.3 Hz, 1H), 8.18 (d, J=7.4 Hz, 1H), 8.03 (dd, J=3.0, 1.4 Hz, 1H), 7.64 (d, J=1.3 Hz, 1H), 7.46 (t, J=7.8 Hz, OH), 7.34 (dd, J=11.7, 2.1 Hz, 1H), 7.20 (d, J=7.4 Hz, 1H), 4.69 (t, J=7.3 Hz, 2H), 3.84-3.74 (m, 1H), 3.60-3.45 (m, 1H), 3.29 (d, J=4.8 Hz, 1H), 3.25-3.07 (m, 2H), 2.50-2.37 (m, 1H), 2.24-2.06 (m, 1H), 1.75 (td, J=7.3, 3.1 Hz, 3H).

### Example 257: Synthesis of Compound 527

### Synthesis of Intermediate C437

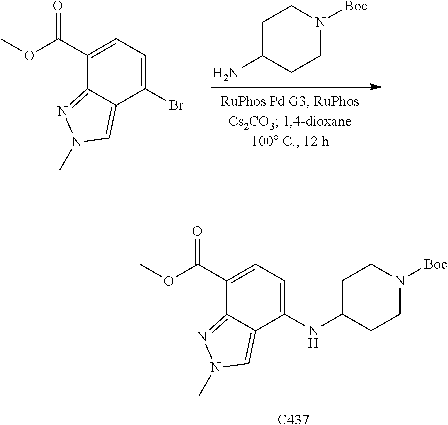 [SMILES: COC(=O)c1ccc(Br)c2cn(C)nc12 ; CC(C)(C)OC(=O)N1CCC(N)CC1 ; COC(=O)c1ccc(NC2CCN(C(=O)OC(C)(C)C)CC2)c2cn(C)nc12 ; "C437"]

To a stirred mixture of methyl 4-bromo-2-methylindazole-7-carboxylate (310 mg, 1.152 mmol, 1 equiv) and tert-butyl 4-aminopiperidine-1-carboxylate (276.8 mg, 1.382 mmol, 1.2 equiv) in 1,4-dioxane (3 mL) were added Cs₂CO₃ (1.12 mg, 3.456 mmol, 3 equiv), RuPhos (107.5 mg, 0.230 mmol, 0.2 equiv) and RuPhos Palladacycle Gen.3 (96.4 mg, 0.115 mmol, 0.1 equiv) at room temperature under nitrogen atmosphere. The resulting mixture was stirred for 12 h at 100° C. under nitrogen atmosphere. The resulting mixture was concentrated under reduced pressure. The residue was purified by silica gel column chromatography, eluted with PE/EA (1:1) to afford methyl 4-{[1-(tert-butoxycarbonyl)piperidin-4-yl]amino}-2-methylindazole-7-carboxylate (300 mg, 67%) as a solid. LCMS (ES, m/z): 389 [M+H]⁺

### Synthesis of Intermediate C438

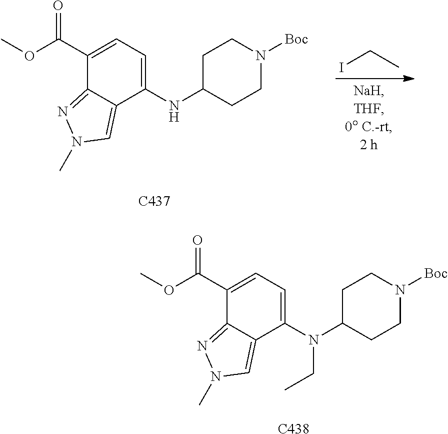

To a solution of methyl 4-{[1-(tert-butoxycarbonyl)piperidin-4-yl]amino}-2-methylindazole-7-carboxylate (110 mg, 0.283 mmol, 1 equiv) in THF (2 mL) was added NaH (17.0 mg, 0.424 mmol, 1.5 equiv, 60%) at 0° C. The mixture was stirred for 30 min and ethyl iodide (44.2 mg, 0.283 mmol, 1.0 equiv) was added and the mixture was allowed to warm to rt and stirred for 2 h. The reaction was quenched with MeOH. The resulting mixture was concentrated under reduced pressure. The residue was purified by silica gel column chromatography, eluted with PE/EA (1:1) to afford methyl 4-{[1-(tert-butoxycarbonyl)piperidin-4-yl](ethyl)amino}-2-methylindazole-7-carboxylate (120 mg, 92%) as a solid. LCMS (ES, m/z): 417 [M+H]⁺

### Synthesis of Intermediate C439

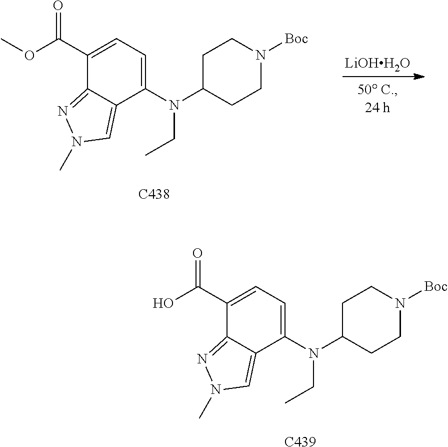

To a stirred mixture of methyl 4-{[1-(tert-butoxycarbonyl)piperidin-4-yl](ethyl)amino}-2-methylindazole-7-carboxylate (120 mg, 0.288 mmol, 1 equiv) in THF (1.5 mL) and H₂O (1.5 mL) was added lithiumol hydrate (96.71 mg, 2.304 mmol, 8 equiv) at room temperature. The resulting mixture was stirred for 2 h at 50° C. The mixture was acidified to pH 5 with HCl (2M). The resulting mixture was extracted with EtOAc (3×5 mL). The combined organic layers were washed with water (3×5 mL), dried over anhydrous Na₂SO₄. After filtration, the filtrate was concentrated under reduced pressure to afford 4-{[1-(tert-butoxycarbonyl)piperidin-4-yl](ethyl)amino}-2-methylindazole-7-carboxylic acid (120 mg, 99%) as a solid. LCMS (ES, m/z): 403 [M+H]⁺

### Synthesis of Intermediate C440

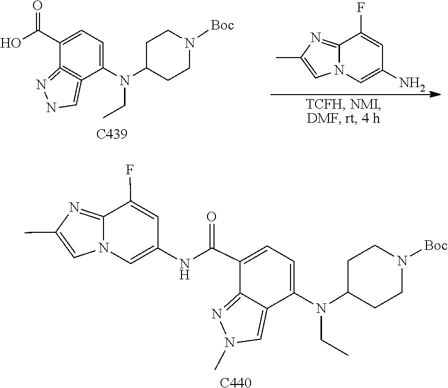

To a stirred mixture of 4-{[1-(tert-butoxycarbonyl)piperidin-4-yl](ethyl)amino}-2-methylindazole-7-carboxylic acid (100 mg, 0.248 mmol, 1 equiv) and 8-fluoro-2-methylimidazo[1,2-a]pyridin-6-amine (49.24 mg, 0.298 mmol, 1.2 equiv) in DMF (1 mL) were added NMI (81.60 mg, 0.992 mmol, 4 equiv) and TCFH (104.57 mg, 0.372 mmol, 1.5 equiv) at room temperature. The resulting mixture was stirred for 4 h at room temperature. The resulting mixture was diluted with water (6 mL). The resulting mixture was extracted with EtOAc (3×6 mL). The combined organic layers were washed with water (3×10 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl 4-{ethyl[7-({8-fluoro-2-methylimidazo[1,2-a]pyridin-6-yl}carbamoyl)-2-methylindazol-4-yl]amino}piperidine-1-carboxylate (50 mg, 37%) as a solid. LCMS (ES, m/z): 550 [M+H]⁺

### Synthesis of Compound 527

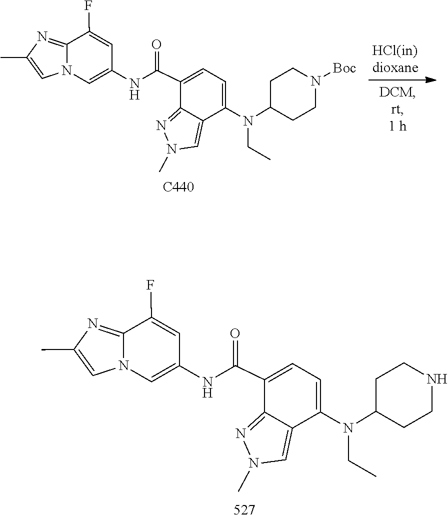

To a stirred mixture of tert-butyl 4-{ethyl[7-({8-fluoro-2-methylimidazo[1,2-a]pyridin-6-yl}carbamoyl)-2-methylindazol-4-yl]amino}piperidine-1-carboxylate (50 mg, 0.091 mmol, 1 equiv) in DCM (1 mL) was added HCl(gas) in 1,4-dioxane (0.2 mL, 4M) dropwise at room temperature. The resulting mixture was stirred for 1 h at room temperature. The resulting mixture was concentrated under reduced pressure. The crude product was purified by Prep-HPLC (Condition 10, Gradient 2) to afford 4-[ethyl(piperidin-4-yl)amino]-N-{8-fluoro-2-methylimidazo[1,2-a]pyridin-6-yl}-2-methylindazole-7-carboxamide (9 mg, 22%) as a solid. LCMS (ES, m/z): 450 [M+H]^{+ 1}H NMR (400 MHz, DMSO-d₆) δ 11.09 (s, 1H), 9.21 (d, J=1.7 Hz, 1H), 8.56 (s, 1H), 7.94 (d, J=8.4 Hz, 1H), 7.89 (d, J=3.1 Hz, 1H), 7.32 (dd, J=12.4, 1.7 Hz, 1H), 6.37 (d, J=8.5 Hz, 1H), 4.31 (s, 3H), 4.06 (d, J=8.9 Hz, 1H), 3.57 (t, J=7.0 Hz, 2H), 3.04 (d, J=12.1 Hz, 2H), 2.76-2.60 (m, 2H), 2.35 (s, 3H), 1.72 (d, J=18.6 Hz, 4H), 1.21 (t, J=6.9 Hz, 3H).

### Example 258: Synthesis of Compound 528

### Synthesis of Intermediate C441

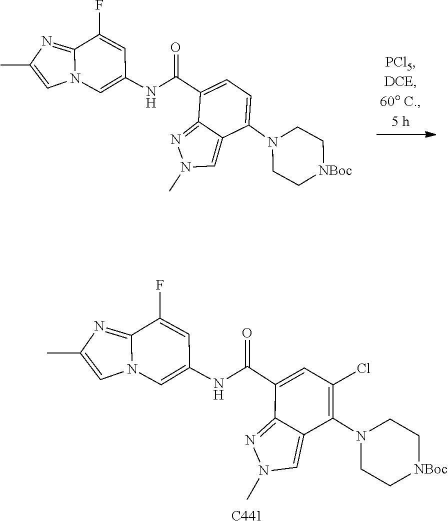

To a stirred mixture of tert-butyl 4-[7-({8-fluoro-2-methylimidazo[1,2-a]pyridin-6-yl}carbamoyl)-2-methylindazol-4-yl]piperazine-1-carboxylate (85.0 mg, 0.167 mmol, 1.0 equiv) in DCE (2 mL) was added PCl₅ (45.3 mg, 0.217 mmol, 1.3 equiv) in portions at room temperature. The resulting mixture was stirred for 5 h at 60° C. The reaction was monitored by LCMS. The resulting mixture was concentrated under reduced pressure. The crude product mixture was used in the next step directly without further purification. LCMS (ES, m/z): 541 [M+H]⁺

### Synthesis of Compound 528

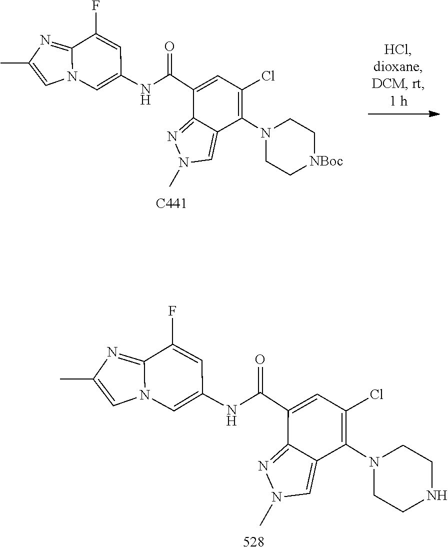

Into a 40 mL vial were added tert-butyl 4-[5-chloro-7-({8-fluoro-2-methylimidazo[1,2-a]pyridin-6-yl} carbamoyl)-2-methylindazol-4-yl]piperazine-1-carboxylate (80.0 mg, 0.148 mmol, 1.0 equiv), DCM (2 mL) and HCl(gas) in 1,4-dioxane (0.5 mL, 4M) at room temperature. The resulting mixture was stirred for 1 h at room temperature. The reaction was monitored by LCMS. The resulting mixture was concentrated under reduced pressure. The crude product was purified by Prep-HPLC (Condition 13, Gradient 1) to afford 5-chloro-N-{8-fluoro-2-methylimidazo[1,2-a]pyridin-6-yl}-2-methyl-4-(piperazin-1-yl) indazole-7-carboxamide (15 mg, 23%) as a solid. LCMS (ES, m/z): 441 [M+H]^{+ 1}H NMR (400 MHz, DMSO-d₆) δ 11.00 (s, 1H), 9.22 (d, J=1.7 Hz, 1H), 8.95 (s, 1H), 7.96-7.90 (m, 2H), 7.36 (dd, J=12.3, 1.7 Hz, 1H), 4.32 (s, 3H), 3.39 (t, J=4.7 Hz, 4H), 2.90 (t, J=4.8 Hz, 4H), 2.36 (s, 3H).

### Example 259: Synthesis of Compound 529

### Synthesis of Intermediate C442

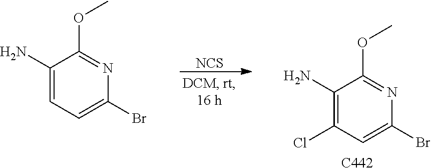

To a stirred mixture of 6-bromo-2-methoxypyridin-3-amine (9 g, 44.326 mmol, 1 equiv) in DCM (90 mL) was added NCS (7.1 g, 53.191 mmol, 1.2 equiv) in portions at 0° C. The resulting mixture was stirred for overnight at room temperature. The resulting mixture was diluted with water (100 mL). The resulting mixture was extracted with CH₂Cl₂ (2×100 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (10:1) to afford 6-bromo-4-chloro-2-methoxypyridin-3-amine (6 g, 57%) as a solid. LCMS (ES, m/z): 237 [M+H]⁺

### Synthesis of Intermediate C443

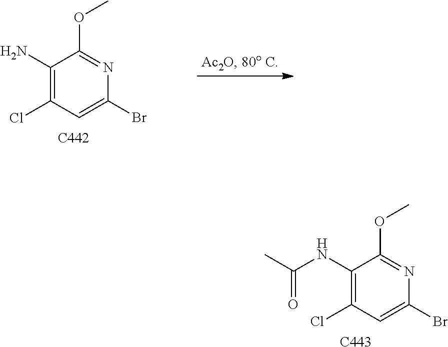

A solution of 6-bromo-4-chloro-2-methoxypyridin-3-amine (2 g, 8.422 mmol, 1 equiv) in Ac₂O (20 mL) was stirred for 2 h at 80° C. The resulting mixture was concentrated under vacuum. The residue was purified by silica gel column chromatography, eluted with PE/EA (1:1) to afford N-acetyl-N-(6-bromo-4-chloro-2-methoxypyridin-3-yl) acetamide (1.5 g, 55%) as a solid. LCMS (ES, m/z): 280 [M+H]⁺

### Synthesis of Intermediate C444

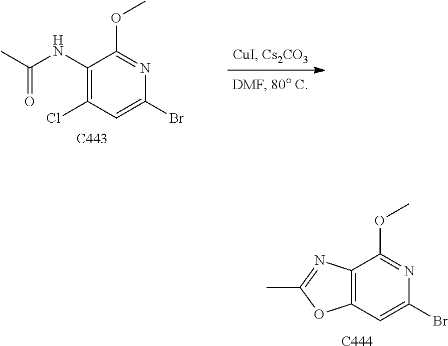

To a solution of N-(6-bromo-4-chloro-2-methoxypyridin-3-yl)acetamide (1 g, 3.578 mmol, 1 equiv) in DMF (3 mL) was added Cs₂CO₃ (2.33 g, 7.156 mmol, 2.0 equiv), (1S,2S)—N1,N2-dimethylcyclohexane-1,2-diamine (0.1 g, 0.716 mmol, 0.2 equiv) and CuI (0.07 g, 0.358 mmol, 0.1 equiv). After stirring for 3 h at 80° C. under a nitrogen atmosphere, the mixture was allowed to cool down to rt. The resulting mixture was purified by reverse flash chromatography (Condition 5, Gradient 3) to afford 6-bromo-4-methoxy-2-methyl-[1,3]oxazolo[4,5-c]pyridine (100 mg, 12%) as a solid. LCMS (ES, m/z): 243 [M+H]⁺

### Synthesis of Intermediate C445

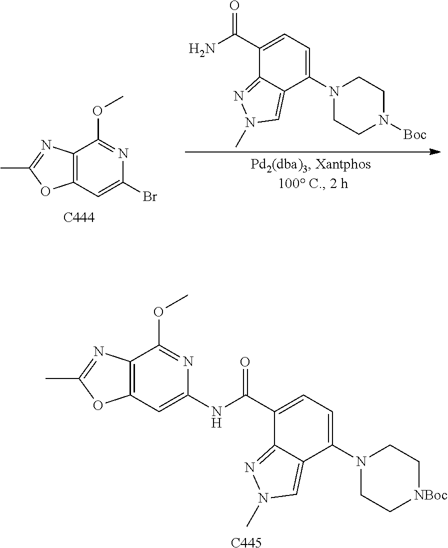

To a solution of tert-butyl 4-(7-carbamoyl-2-methylindazol-4-yl)piperazine-1-carboxylate (150 mg, 0.417 mmol, 1 equiv) and 6-bromo-4-methoxy-2-methyl-[1,3]oxazolo[4,5-c]pyridine (101.4 mg, 0.417 mmol, 1.0 equiv) in dioxane (4 mL) were added Cs₂CO₃ (407.9 mg, 1.251 mmol, 3.0 equiv), Xantphos (48.3 mg, 0.083 mmol, 0.2 equiv) and Pd₂(dba)₃·CHCl₃ (43.2 mg, 0.042 mmol, 0.1 equiv). After stirring for 2 h at 100° C. under a nitrogen atmosphere, the resulting mixture was concentrated under reduced pressure. The residue was purified by reverse flash chromatography (Condition 5, Gradient 2) to afford tert-butyl 4-[7-({4-methoxy-2-methyl-[1,3]oxazolo[4,5-c]pyridin-6-yl}carbamoyl)-2-methylindazol-4-yl]piperazine-1-carboxylate (163 mg, 75%) as a solid. LCMS (ES, m/z): 522 [M+H]⁺

### Synthesis of Compound 529

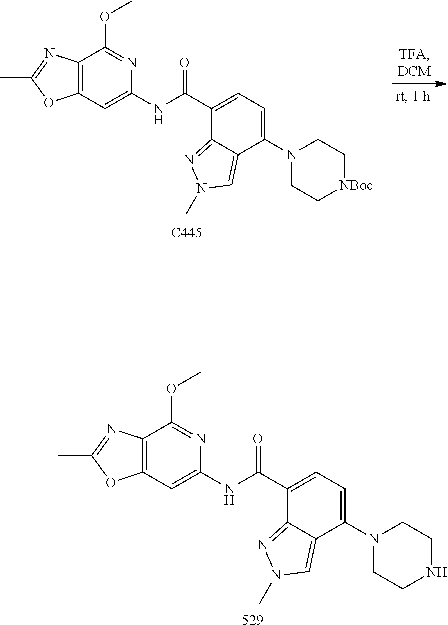

A solution of tert-butyl 4-[7-({4-methoxy-2-methyl-[1,3]oxazolo[4,5-c]pyridin-6-yl}carbamoyl)-2-methylindazol-4-yl]piperazine-1-carboxylate (150 mg, 0.288 mmol, 1 equiv) and TFA (0.3 mL) in DCM (3 mL) was stirred for 1 h at room temperature. The mixture was basified to pH 8 with NH₃(g) in MeOH. The resulting mixture was concentrated under reduced pressure. The residue was purified by reverse flash chromatography (Condition 5, Gradient 3) to afford N-{4-methoxy-2-methyl-[1,3]oxazolo[4,5-c]pyridin-6-yl}-2-methyl-4-(piperazin-1-yl) indazole-7-carboxamide (26 mg, 21%) as a solid. LCMS (ES, m/z): 422 [M+H]^{+ 1}H NMR (300 MHz, DMSO-d₆) δ 11.69 (s, 1H), 8.81 (s, 1H), 8.20 (s, 1H), 8.03 (d, J=8.1 Hz, 1H), 6.50 (d, J=8.2 Hz, 1H), 4.26 (s, 3H), 4.09 (s, 3H), 3.37-3.32 (m, 4H), 2.92 (t, J=5.0 Hz, 4H), 2.60 (s, 3H).

### Example 260: Synthesis of Compound 531

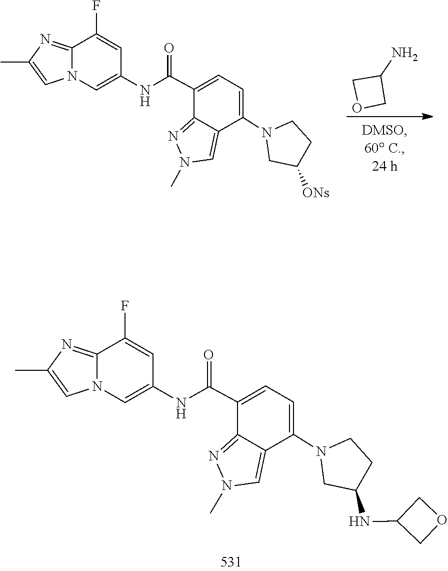

To a stirred mixture of (3S)-1-[7-({8-fluoro-2-methylimidazo[1,2-a]pyridin-6-yl}carbamoyl)-2-methylindazol-4-yl]pyrrolidin-3-yl 4-nitrobenzenesulfonate (150 mg, 0.253 mmol, 1 equiv) in DMSO (0.75 mL) was added oxetan-3-amine (92.36 mg, 1.265 mmol, 5 equiv) dropwise at room temperature. The resulting mixture was stirred for 24 h at 60° C. The resulting mixture was diluted with water (10 mL). The resulting mixture was extracted with EtOAc (2×10 mL). The combined organic layers were washed with water (1×20 mL), dried over anhydrous Na₂SO₄. After filtration, the filtrate was concentrated under reduced pressure. The residue was purified by silica gel column chromatography, eluted with CH₂Cl₂/MeOH (10:1) to afford N-{8-fluoro-2-methylimidazo[1,2-a]pyridin-6-yl}-2-methyl-4-[(3R)-3-(oxetan-3-ylamino)pyrrolidin-1-yl]indazole-7-carboxamide (50 mg) and then prep-chiral-HPLC (Condition 9, Gradient 1) to afford the target compound (12.2 mg, 10%) as a solid. LCMS (ES, m/z): 464 [M+H]^{+ 1}H NMR (300 MHz, DMSO-d₆) δ 11.01 (s, 1H), 9.20 (d, J=1.7 Hz, 1H), 8.82 (s, 1H), 7.97-7.85 (m, 2H), 7.31 (dd, J=12.4, 1.6 Hz, 1H), 6.01 (d, J=8.4 Hz, 1H), 4.68 (td, J=6.6, 3.7 Hz, 2H), 4.36 (dt, J=10.6, 6.1 Hz, 2H), 4.27 (s, 3H), 4.03 (p, J=6.7 Hz, 1H), 3.79-3.71 (m, 2H), 3.67-3.61 (m, 1H), 3.41 (d, J=8.8 Hz, 3H), 2.35 (s, 3H), 2.14-2.09 (m, 1H), 1.85 (dd, J=12.3, 6.3 Hz, 1H).

### Example 261: Synthesis of Compound 533

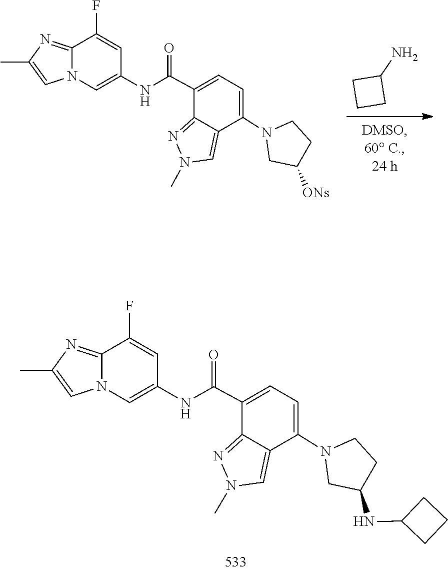

To a stirred mixture of (3S)-1-[7-({8-fluoro-2-methylimidazo[1,2-a]pyridin-6-yl}carbamoyl)-2-methylindazol-4-yl]pyrrolidin-3-yl 4-nitrobenzenesulfonate (150 mg, 0.253 mmol, 1 equiv) in DMSO (0.75 mL) was added cyclobutanamine (89.83 mg, 1.265 mmol, 5 equiv) dropwise at room temperature. The resulting mixture was stirred for 24 h at 60° C. The resulting mixture was diluted with water (10 mL). The resulting mixture was extracted with EtOAc (2×10 mL). The combined organic layers were washed with water (1×20 mL), dried over anhydrous Na₂SO₄. After filtration, the filtrate was concentrated under reduced pressure. The residue was purified by silica gel column chromatography, eluted with CH₂Cl₂/MeOH (10:1) to afford (R)-4-(3-(cyclobutylamino)pyrrolidin-1-yl)-N-(8-fluoro-2-methylimidazo[1,2-a]pyridin-6-yl)-2-methyl-2H-indazole-7-carboxamide (60 mg) and then prep-chiral-HPLC (Condition 9, Gradient 1) to afford the target compound (19.3 mg, 17%) as a solid. LCMS (ES, m/z): 462 [M+H]^{+ 1}H NMR (400 MHz, DMSO-d₆) δ 11.01 (s, 1H), 9.19 (d, J=1.6 Hz, 1H), 8.81 (s, 1H), 7.93 (d, J=8.2 Hz, 1H), 7.88 (d, J=3.0 Hz, 1H), 7.30 (dd, J=12.4, 1.7 Hz, 1H), 6.01 (d, J=8.4 Hz, 1H), 4.27 (s, 3H), 3.78-3.72 (m, 2H), 3.66-3.59 (m, 1H), 3.49-3.47 (m, 1H), 3.41-3.39 (m, 2H), 2.35 (s, 3H), 2.21-2.11 (m, 3H), 1.95-1.88 (m, 1H), 1.79-1.74 (m, 2H), 1.66-1.55 (m, 2H).

### Example 262: Synthesis of Compound 535

### Synthesis of Intermediate C446

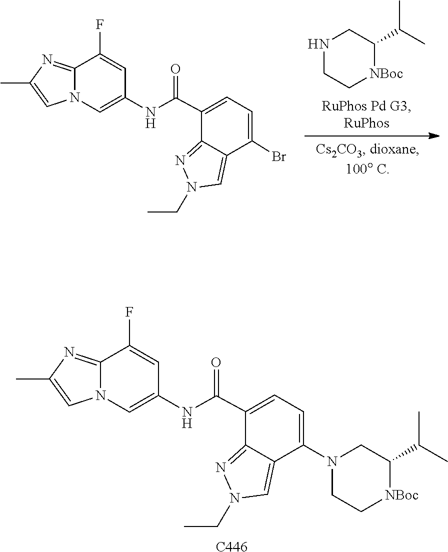

To a stirred solution of 4-bromo-2-ethyl-N-{8-fluoro-2-methylimidazo[1,2-a]pyridin-6-yl}indazole-7-carboxamide (150 mg, 0.36 mmol, 1 equiv) and tert-butyl (2S)-2-isopropylpiperazine-1-carboxylate (124 mg, 0.54 mmol, 1.5 equiv) in dioxane (8 mL) were added Cs₂CO₃ (235 mg, 0.72 mmol, 2 equiv), RuPhos (34 mg, 0.072 mmol, 0.2 equiv) and RuPhos Palladacycle Gen.3 (31 mg, 0.036 mmol, 0.1 equiv) at room temperature. The resulting mixture was stirred for 2 h at 100° C. under nitrogen atmosphere. The mixture was allowed to cool down to room temperature. The resulting mixture was concentrated under vacuum. The residue was purified by silica gel column chromatography, eluted with PE/EA (1:5) to afford tert-butyl (2S)-4-[2-ethyl-7-({8-fluoro-2-methylimidazo[1,2-a]pyridin-6-yl}carbamoyl) indazol-4-yl]-2-isopropylpiperazine-1-carboxylate (100 mg, 49%) as a solid. LCMS (ES, m/z): 564 [M+H]⁺

### Synthesis of Compound 535

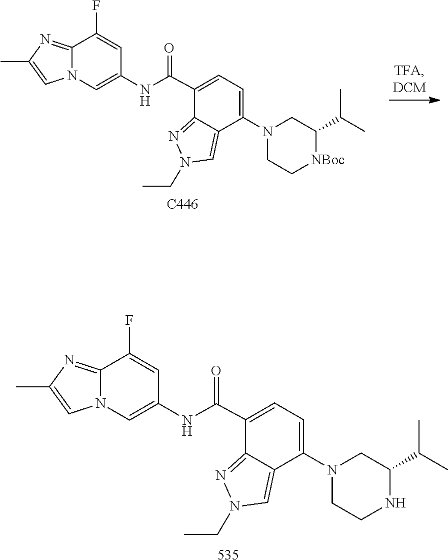

A solution of tert-butyl (2S)-4-[2-ethyl-7-({8-fluoro-2-methylimidazo[1,2-a]pyridin-6-yl}carbamoyl) indazol-4-yl]-2-isopropylpiperazine-1-carboxylate (100 mg, 0.177 mmol, 1 equiv) in TFA (1 mL) and DCM (3 mL) was stirred for 3 h at room temperature. The resulting mixture was concentrated under reduced pressure. The residue was basified to pH 8 with 7 M NH₃(g) in MeOH. The resulting mixture was concentrated under reduced pressure. The residue was purified by reverse flash chromatography (Condition 3, Gradient 2) to afford 2-ethyl-N-{8-fluoro-2-methylimidazo[1,2-a]pyridin-6-yl}-4-[(3 S)-3-isopropylpiperazin-1-yl]indazole-7-carboxamide (35 mg, 43%) as a solid. LCMS (ES, m/z): 464 [M+H]^{+ 1}H NMR (300 MHz, DMSO-d₆) δ 11.10 (s, 1H), 9.21 (d, J=1.7 Hz, 1H), 8.76 (s, 1H), 7.98 (d, J=8.1 Hz, 1H), 7.90 (d, J=3.1 Hz, 1H), 7.30 (d, J=12.2 Hz, 1H), 6.50 (d, J=8.2 Hz, 1H), 4.60 (q, J=7.2 Hz, 2H), 3.84 (d, J=8.4 Hz, 1H), 3.75 (d, J=11.7 Hz, 1H), 3.05 (d, J=8.0 Hz, 1H), 2.90 (d, J=8.6 Hz, 2H), 2.67 (t, J=11.0 Hz, 2H), 2.35 (s, 3H), 1.61 (t, J=7.2 Hz, 4H), 0.97 (d, J=6.7 Hz, 6H).

### Example 263: Synthesis of Compounds 537 and 538

### Synthesis of Compound 537

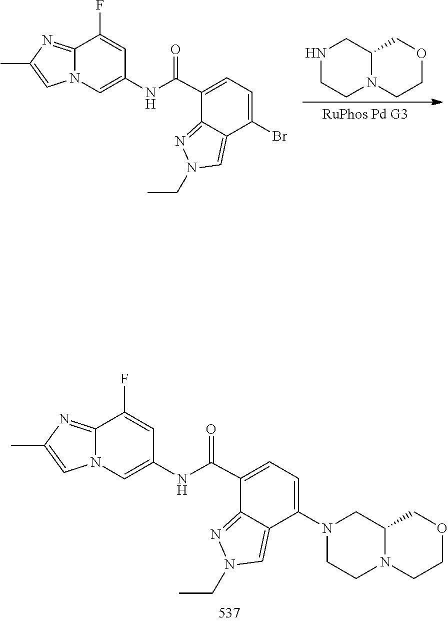 [SMILES: CCn1cc2c(Br)ccc(C(=O)Nc3cc(F)c4nc(C)cn4c3)c2n1 ; C1CN2CCOC[C@H]2CN1 ; CCn1cc2c(N3CCN4CCOC[C@H]4C3)ccc(C(=O)Nc3cc(F)c4nc(C)cn4c3)c2n1]

To a stirred solution of 4-bromo-2-ethyl-N-{8-fluoro-2-methylimidazo[1,2-a]pyridin-6-yl}indazole-7-carboxamide (150 mg, 0.360 mmol, 1 equiv), Cs₂CO₃ (352 mg, 1.080 mmol, 3 equiv) and (9aR)-octahydropyrazino[2,1-c][1,4]oxazine (76 mg, 0.540 mmol, 1.5 equiv) in 1,4-dioxane (5 mL) were added RuPhos (33 mg, 0.072 mmol, 0.2 equiv) and RuPhos Palladacycle Gen.3 (30 mg, 0.036 mmol, 0.1 equiv) at room temperature under nitrogen atmosphere. The resulting mixture was stirred for 5 h at 100° C. The resulting mixture was concentrated under reduced pressure. The residue was purified by reverse flash chromatography (Condition 3, Gradient 3) to afford 4-[(9aR)-hexahydro-1H-pyrazino[2,1-c][1,4]oxazin-8-yl]-2-ethyl-N-{8-fluoro-2-methylimidazo[1,2-a]pyridin-6-yl}indazole-7-carboxamide (11 mg, 6%) as a solid. LCMS (ES, m/z): 478 [M+H]^{+ 1}H NMR (400 MHz, DMSO-d₆) δ 11.12 (s, 1H), 9.22 (d, J=1.6 Hz, 1H), 8.89 (s, 1H), 7.99 (d, J=8.0 Hz, 1H), 7.92 (d, J=3.1 Hz, 1H), 7.33 (d, J=12.0 Hz, 1H), 6.52 (s, 1H), 4.60 (q, J=7.3 Hz, 2H), 3.86 (s, 4H), 3.58 (s, 4H), 2.97 (s, 1H), 2.87 (s, 1H), 2.73 (s, 1H), 2.54 (s, 1H), 2.36 (s, 3H), 1.63 (t, J=7.3 Hz, 3H).

### Synthesis of Compound 538

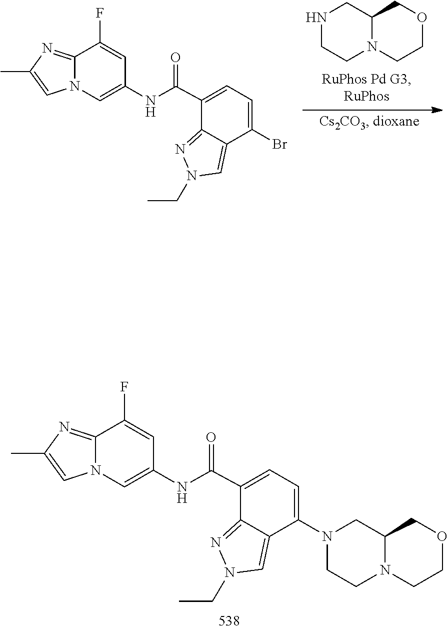 [SMILES: CCn1cc2c(Br)ccc(C(=O)Nc3cc(F)c4nc(C)cn4c3)c2n1 ; C1CN2CCOC[C@@H]2CN1 ; CCn1cc2c(N3CCN4CCOC[C@@H]4C3)ccc(C(=O)Nc3cc(F)c4nc(C)cn4c3)c2n1]

To a stirred mixture of 4-bromo-2-ethyl-N-{8-fluoro-2-methylimidazo[1,2-a]pyridin-6-yl}indazole-7-carboxamide (100 mg, 0.24 mmol, 1 equiv) and (9aS)-octahydropyrazino[2,1-c][1,4]oxazine (69 mg, 0.48 mmol, 2 equiv) in dioxane (3 mL) were added Cs₂CO₃ (470 mg, 1.440 mmol, 6 equiv), RuPhos (23 mg, 0.048 mmol, 0.2 equiv) and RuPhos Palladacycle Gen.3 (20 mg, 0.024 mmol, 0.1 equiv) at room temperature. The resulting mixture was stirred for 5 h at 100° C. The mixture was allowed to cool down to room temperature. The reaction was quenched with water (20 mL) at room temperature. The resulting mixture was extracted with EtOAc (3×5 mL). The combined organic layers were washed with brine (1×5 mL), dried over anhydrous Na₂SO₄. After filtration, the filtrate was concentrated under reduced pressure. The residue was purified by reverse flash chromatography (Condition 3, Gradient 1) to afford 4-[(9aS)-hexahydro-1H-pyrazino[2,1-c][1,4]oxazin-8-yl]-2-ethyl-N-{8-fluoro-2-methylimidazo[1,2-a]pyridin-6-yl} indazole-7-carboxamide (55 mg, 48%) as a solid. LCMS (ES, m/z): 478 [M+H]^{+ 1}H NMR (300 MHz, DMSO-d₆) δ 11.11 (s, 1H), 9.21 (d, J=1.7 Hz, 1H), 8.88 (s, 1H), 7.98 (d, J=8.1 Hz, 1H), 7.91 (d, J=3.1 Hz, 1H), 7.31 (dd, J=12.4, 1.5 Hz, 1H), 6.51 (d, J=8.1 Hz, 1H), 4.60 (q, J=7.2 Hz, 2H), 3.81 (dq, J=22.0, 12.0 Hz, 4H), 3.57 (t, J=11.2 Hz, 1H), 3.19 (t, J=10.4 Hz, 1H), 3.08 (t, J=11.5 Hz, 1H), 2.88 (d, J=11.5 Hz, 1H), 2.73 (d, J=10.7 Hz, 1H), 2.61 (t, J=11.2 Hz, 1H), 2.35 (s, 6H), 1.63 (t, J=7.2 Hz, 3H).

### Example 264: Synthesis of Compound 539

### Synthesis of Intermediate C447

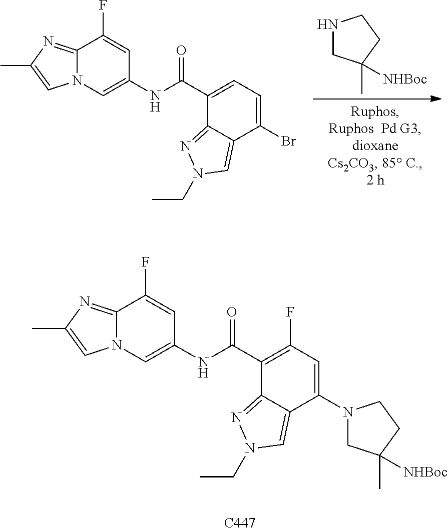

To a solution of 4-bromo-2-ethyl-N-{8-fluoro-2-methylimidazo[1,2-a]pyridin-6-yl}indazole-7-carboxamide (100.0 mg, 0.240 mmol, 1.0 equiv) and tert-butyl N-(3-methylpyrrolidin-3-yl)carbamate (72.1 mg, 0.360 mmol, 1.5 equiv) in dioxane (4 mL) were added Cs₂CO₃ (156.5 mg, 0.480 mmol, 2.0 equiv) and Ruphos (22.4 mg, 0.048 mmol, 0.2 equiv), RuPhos Palladacycle Gen.3 (20.0 mg, 0.024 mmol, 0.1 equiv). After stirring for 2 h at 80° C. under a nitrogen atmosphere, the resulting mixture was concentrated under reduced pressure. The residue was purified by silica gel column chromatography, eluted with PE/EA (1:5) to afford tert-butyl N-{1-[2-ethyl-7-({8-fluoro-2-methylimidazo[1,2-a]pyridin-6-yl}carbamoyl) indazol-4-yl]-3-methylpyrrolidin-3-yl}carbamate (80 mg, 57.20%) as a solid. LCMS (ES, m/z): 536 [M+H]⁺

### Synthesis of Compound 539

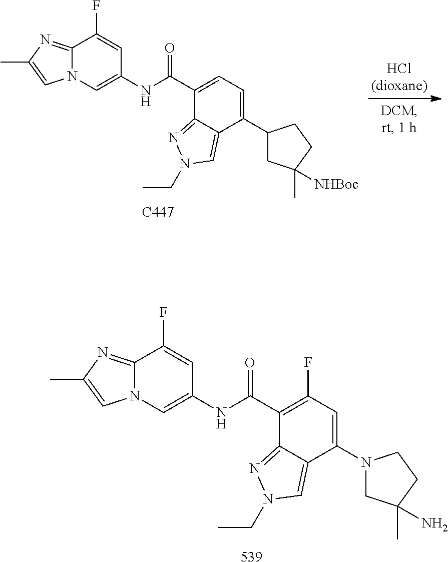

To a stirred mixture of tert-butyl N-{1-[2-ethyl-7-({8-fluoro-2-methylimidazo[1,2-a]pyridin-6-yl} carbamoyl) indazol-4-yl]-3-methylpyrrolidin-3-yl}carbamate (80.0 mg, 0.149 mmol, 1.0 equiv) in DCM (2 mL) was added HCl(gas) in 1,4-dioxane (1 mL) dropwise at room temperature. The resulting mixture was stirred for 1 h at room temperature. The resulting mixture was concentrated under reduced pressure. The crude product was purified by Prep-HPLC (Condition 13, Gradient 1) to afford 4-(3-amino-3-methylpyrrolidin-1-yl)-2-ethyl-N-{8-fluoro-2-methylimidazo[1,2-a]pyridin-6-yl}indazole-7-carboxamide (15 mg, 23%) as a solid. LCMS (ES, m/z): 436 [M+H]^{+ 1}H NMR (400 MHz, DMSO-d₆) δ 11.05 (s, 1H), 9.20 (d, J=1.7 Hz, 1H), 8.83 (s, 1H), 7.93 (d, J=8.3 Hz, 1H), 7.89 (d, J=3.0 Hz, 1H), 7.27 (dd, J=12.4, 1.7 Hz, 1H), 6.00 (d, J=8.4 Hz, 1H), 4.57 (q, J=7.3 Hz, 2H), 3.85 (d, J=8.9 Hz, 1H), 3.72-3.71 (m, 1H), 3.51 (q, J=10.1 Hz, 2H), 2.37-2.33 (m, 3H), 1.96 (t, J=6.9 Hz, 2H), 1.61 (t, J=7.3 Hz, 3H), 1.34 (s, 3H).

### Example 265: Synthesis of Compounds 541 and 542

### Synthesis of Intermediate C448

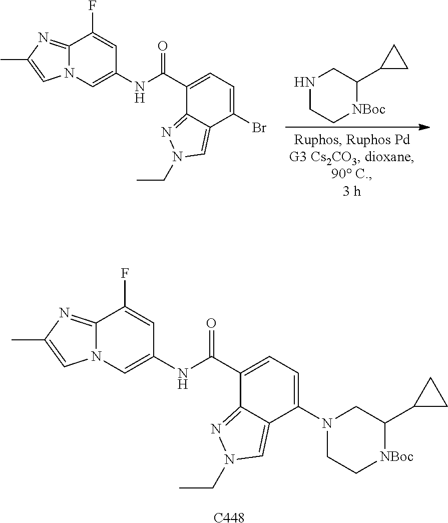

To a stirred mixture of 4-bromo-2-ethyl-N-{8-fluoro-2-methylimidazo[1,2-a]pyridin-6-yl}indazole-7-carboxamide (150 mg, 0.360 mmol, 1.0 equiv) and tert-butyl 2-cyclopropylpiperazine-1-carboxylate (97.8 mg, 0.432 mmol, 1.2 equiv) in dioxane (1.5 mL) were added Cs₂CO₃ (352.2 mg, 1.080 mmol, 3.0 equiv), Ruphos (33.6 mg, 0.072 mmol, 0.2 equiv) and RuPhos Palladacycle Gen.3 (30.1 mg, 0.036 mmol, 0.1 equiv) at room temperature. The resulting mixture was stirred for 3 h at 90° C. under nitrogen atmosphere. The mixture was allowed to cool down to room temperature. The reaction was concentrated off. The residue was purified by silica gel column chromatography, eluted with CH₂Cl₂/MeOH (10:1) to afford tert-butyl 2-cyclopropyl-4-[2-ethyl-7-({8-fluoro-2-methylimidazo[1,2-a]pyridin-6-yl}carbamoyl) indazol-4-yl]piperazine-1-carboxylate (116 mg, 57%) as a solid. LCMS (ES, m/z): 562 [M+H]⁺

### Synthesis of Compound 541

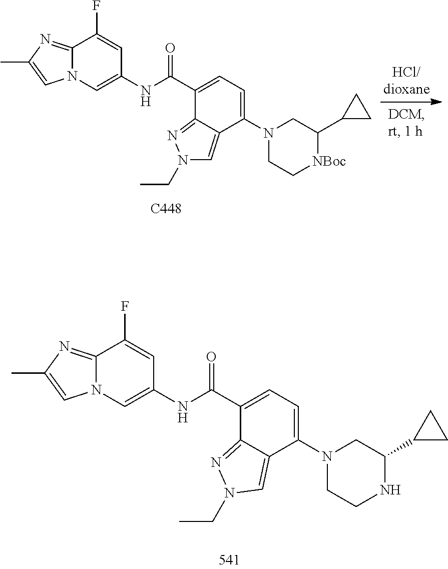

A solution of tert-butyl 2-cyclopropyl-4-[2-ethyl-7-({8-fluoro-2-methylimidazo[1,2-a]pyridin-6-yl}carbamoyl) indazol-4-yl]piperazine-1-carboxylate (110 mg, 0.196 mmol, 1 equiv) in DCM (0.9 mL) was treated with HCl(gas) in 1,4-dioxane (0.3 mL). The resulting mixture was stirred for 1 h at room temperature. The resulting mixture was concentrated under reduced pressure. The residue was neutralized to PH=7 with NaHCO₃ aqueous and extracted with DCM/MeOH (20/1). The organic layer was concentrated in vacuo. The residue was purified by chiral-HPLC (Condition 2, Gradient 2) to afford 4-[(3S)-3-cyclopropylpiperazin-1-yl]-2-ethyl-N-{8-fluoro-2-methylimidazo[1,2-a]pyridin-6-yl}indazole-7-carboxamide (23 mg, 29%) as a solid. LCMS (ES, m/z): 462 [M+H]^{+ 1}H NMR (400 MHz, DMSO-d₆) δ 11.11 (s, 1H), 9.21 (d, J=1.7 Hz, 1H), 8.77 (s, 1H), 7.99 (d, J=8.1 Hz, 1H), 7.91 (d, J=3.1 Hz, 1H), 7.30 (dd, J=12.3, 1.7 Hz, 1H), 6.51 (d, J=8.2 Hz, 1H), 4.61 (q, J=7.3 Hz, 2H), 3.79 (dd, J=19.6, 11.7 Hz, 2H), 3.08-2.72 (m, 4H), 2.35 (s, 3H), 2.16-2.03 (m, 1H), 1.62 (t, J=7.3 Hz, 3H), 0.81 (qt, J=8.4, 5.0 Hz, 1H), 0.44 (dd, J=7.9, 3.9 Hz, 2H), 0.42-0.26 (m, 2H).

### Synthesis of Compound 542

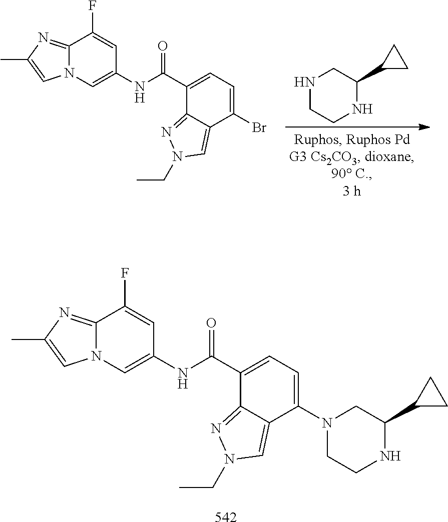

To a stirred mixture of 4-bromo-2-ethyl-N-{8-fluoro-2-methylimidazo[1,2-a]pyridin-6-yl}indazole-7-carboxamide (100 mg, 0.240 mmol, 1.0 equiv) and (2R)-2-cyclopropylpiperazine (36.4 mg, 0.288 mmol, 1.2 equiv) in dioxane (1 mL) were added Cs₂CO₃ (244.2 mg, 0.750 mmol, 3.0 equiv), Ruphos (23.3 mg, 0.050 mmol, 0.2 equiv) and RuPhos Palladacycle Gen.3 (20.9 mg, 0.025 mmol, 0.1 equiv) at room temperature. The resulting mixture was stirred for 3 h at 90° C. under nitrogen atmosphere. The mixture was allowed to cool down to room temperature and concentrated off. The crude product was purified by Prep-HPLC (Condition 10, Gradient 2) to afford 4-[(3R)-3-cyclopropylpiperazin-1-yl]-2-ethyl-N-{8-fluoro-2-methylimidazo[1,2-a]pyridin-6-yl}indazole-7-carboxamide (32.3 mg, 29%) as a solid. LCMS (ES, m/z): 462 [M+H]^{+ 1}H NMR (400 MHz, DMSO-d₆) δ 11.11 (s, 1H), 9.21 (d, J=1.6 Hz, 1H), 8.77 (s, 1H), 7.99 (d, J=8.1 Hz, 1H), 7.91 (d, J=3.0 Hz, 1H), 7.30 (dd, J=12.3, 1.7 Hz, 1H), 6.51 (d, J=8.2 Hz, 1H), 4.61 (q, J=7.3 Hz, 2H), 3.79 (dd, J=19.5, 11.6 Hz, 2H), 3.03 (d, J=11.2 Hz, 1H), 2.99-2.90 (m, 1H), 2.87 (dd, J=11.2, 2.5 Hz, 1H), 2.84-2.75 (m, 1H), 2.35 (s, 3H), 2.11 (t, J=9.1 Hz, 1H), 1.62 (t, J=7.3 Hz, 3H), 0.81 (td, J=8.7, 8.1, 4.1 Hz, 1H), 0.43 (dd, J=7.8, 3.7 Hz, 2H), 0.40-0.26 (m, 2H).

### Example 266: Synthesis of Compound 543

### Synthesis of Intermediate C449

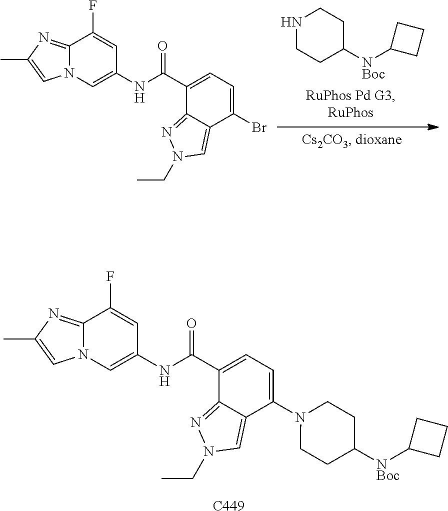

To a stirred solution of 4-bromo-2-ethyl-N-{8-fluoro-2-methylimidazo[1,2-a]pyridin-6-yl}indazole-7-carboxamide (130 mg, 0.312 mmol, 1 equiv) and tert-butyl N-cyclobutyl-N-(piperidin-4-yl)carbamate (159 mg, 0.624 mmol, 2 equiv) in dioxane (10 mL) were added Cs₂CO₃ (204 mg, 0.624 mmol, 2 equiv), RuPhos (29 mg, 0.062 mmol, 0.2 equiv) and RuPhos Palladacycle Gen.3 (26 mg, 0.031 mmol, 0.1 equiv) at room temperature under nitrogen atmosphere. The resulting mixture was stirred for 3 h at 100° C. under nitrogen atmosphere. The mixture was allowed to cool down to room temperature. The resulting mixture was concentrated under reduced pressure. The residue was purified by silica gel column chromatography, eluted with PE/EA (1:10) to afford tert-butyl N-cyclobutyl-N-{1-[2-ethyl-7-({8-fluoro-2-methylimidazo[1,2-a]pyridin-6-yl}carbamoyl) indazol-4-yl]piperidin-4-yl}carbamate (100 mg, 54%) as a solid. LCMS (ES, m/z): 590 [M+H]⁺

### Synthesis of Compound 543

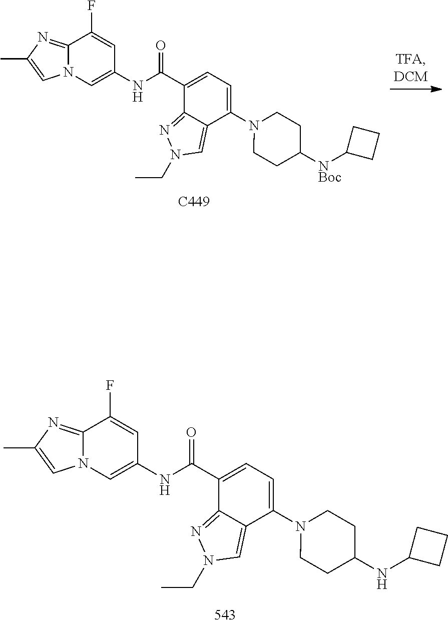

A solution of tert-butyl N-cyclobutyl-N-{1-[2-ethyl-7-({8-fluoro-2-methylimidazo[1,2-a]pyridin-6-yl}carbamoyl) indazol-4-yl]piperidin-4-yl}carbamate (90 mg, 0.153 mmol, 1 equiv) in TFA (2 mL) and DCM (2 mL) was stirred for 2 h at room temperature. The resulting mixture was concentrated under vacuum. The residue was basified to pH 8 with 7M NH₃(g) in MeOH. The resulting mixture was concentrated under reduced pressure. The residue was purified by reverse flash chromatography (Condition 3, Gradient 3) to afford 4-[4-(cyclobutylamino)piperidin-1-yl]-2-ethyl-N-{8-fluoro-2-methylimidazo[1,2-a]pyridin-6-yl}indazole-7-carboxamide (40 mg, 54%) as a solid. LCMS (ES, m/z): 490 [M+H]⁺1H NMR (400 MHz, DMSO-d₆) δ 11.10 (s, 1H), 9.20 (s, 1H), 8.78 (s, 1H), 7.96 (d, J=8.0 Hz, 1H), 7.90 (d, J=3.1 Hz, 1H), 7.29 (d, J=12.2 Hz, 1H), 6.47 (d, J=8.2 Hz, 1H), 4.59 (q, J=7.3 Hz, 2H), 3.86 (d, J=12.5 Hz, 2H), 3.27 (d, J=7.4 Hz, 1H), 3.03 (t, J=11.9 Hz, 2H), 2.65 (dq, J=9.7, 4.6, 4.1 Hz, 1H), 2.35 (s, 3H), 2.12 (t, J=8.2 Hz, 2H), 1.96-1.76 (m, 3H), 1.76-1.50 (m, 7H), 1.43 (q, J=11.5, 9.7 Hz, 2H).

### Example 267: Synthesis of Compounds 544, 547, and 548

### Synthesis of Intermediate C450

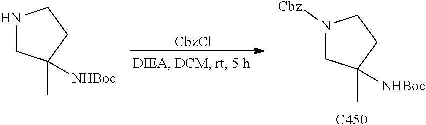

To a stirred solution of tert-butyl N-(3-methylpyrrolidin-3-yl)carbamate (2.0 g, 9.986 mmol, 1.0 equiv) and DIEA (2.5 g, 19.972 mmol, 2.0 equiv) in DCM (30 mL) was added benzyl chloroformate (2.0 g, 11.983 mmol, 1.2 equiv) dropwise at 0° C. under nitrogen atmosphere. The resulting mixture was stirred for 5 h at room temperature. The reaction was monitored by LCMS. The resulting mixture was diluted with deionized water (50 mL). The resulting mixture was extracted with CH₂Cl₂ (2×50 mL). The combined organic layers were washed with brine (1×50 mL), dried over anhydrous Na₂SO₄. After filtration, the filtrate was concentrated under reduced pressure. The resulting mixture was concentrated under reduced pressure. The residue was purified by silica gel column chromatography, eluted with PE/EA (5:1) to afford benzyl 3-[(tert-butoxycarbonyl)amino]-3-methylpyrrolidine-1-carboxylate (3.4 g, 92%) as a solid. LCMS (ES, m/z): 335 [M+H]⁺

### Synthesis of Intermediate C451

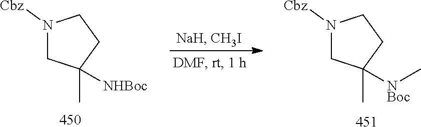

To a stirred solution of benzyl 3-[(tert-butoxycarbonyl)amino]-3-methylpyrrolidine-1-carboxylate (750.0 mg, 2.243 mmol, 1.0 equiv) and NaH (107.6 mg, 4.486 mmol, 2.0 equiv) in dimethylformamide (15 mL) was added methyl iodide (636.6 mg, 4.486 mmol, 2.0 equiv) dropwise at 0° C. The resulting mixture was stirred for 1 h at room temperature. The reaction was quenched with Water at 0° C. The resulting mixture was extracted with EtOAc (2×50 mL). The combined organic layers were washed with brine (1×50 mL), dried over anhydrous Na₂SO₄. After filtration, the filtrate was concentrated under reduced pressure. LCMS (ES, m/z): 349 [M+H]⁺

### Synthesis of Intermediate C452

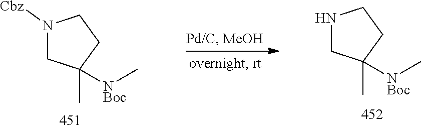

To a solution of benzyl 3-[(tert-butoxycarbonyl)(methyl)amino]-3-methylpyrrolidine-1-carboxylate (650.0 mg, 1.865 mmol, 1.0 equiv) in 15 mL MeOH was added Pd/C (10%, 59.5 mg) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure. This resulted in tert-butyl N-methyl-N-(3-methylpyrrolidin-3-yl) carbamate (350 mg, 79%) as an oil. LCMS (ES, m/z): 215 [M+H]⁺

### Synthesis of Intermediate C453

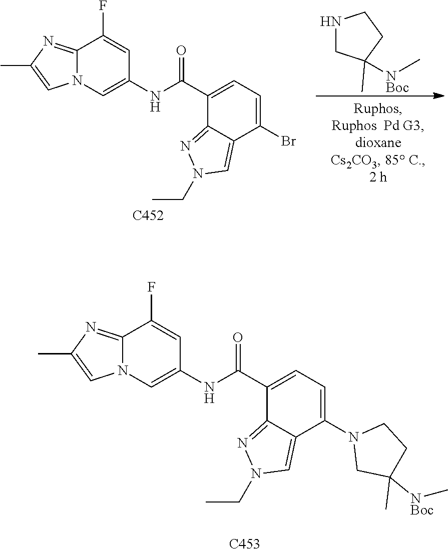

To a solution of 4-bromo-2-ethyl-N-{8-fluoro-2-methylimidazo[1,2-a]pyridin-6-yl}indazole-7-carboxamide (100.0 mg, 0.240 mmol, 1.0 equiv) and tert-butyl N-methyl-N-(3-methylpyrrolidin-3-yl) carbamate (77.2 mg, 0.360 mmol, 1.5 equiv) in dioxane (3 mL) were added Cs₂CO₃ (157.0 mg, 0.480 mmol, 2.0 equiv) and Ruphos (22.4 mg, 0.048 mmol, 0.2 equiv), RuPhos Palladacycle Gen.3 (20.0 mg, 0.024 mmol, 0.1 equiv). After stirring for 2 h at 85° C. under a nitrogen atmosphere, the resulting mixture was concentrated under reduced pressure. The residue was purified by silica gel column chromatography, eluted with PE/EA (1:5) to afford tert-butyl N-{1-[2-ethyl-7-({8-fluoro-2-methylimidazo[1,2-a]pyridin-6-yl}carbamoyl) indazol-4-yl]-3-methylpyrrolidin-3-yl}-N-methylcarbamate (130 mg, 89%) as a solid. LCMS (ES, m/z): 550 [M+H]⁺

### Synthesis of Compound 544

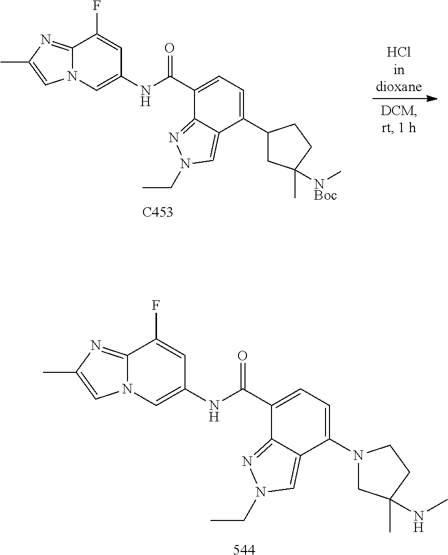

To a stirred solution of tert-butyl N-{1-[2-ethyl-7-({8-fluoro-2-methylimidazo[1,2-a]pyridin-6-yl} carbamoyl) indazol-4-yl]-3-methylpyrrolidin-3-yl}-N-methylcarbamate (100.0 mg, 0.182 mmol, 1.0 equiv) in DCM (2 mL) was added HCl (gas) in 1,4-dioxane (1 mL) dropwise at room temperature. The resulting mixture was stirred for 1 h at room temperature. The reaction was monitored by LCMS. The crude product was purified by Prep-HPLC (Condition 13, Gradient 1) to afford 2-ethyl-N-{8-fluoro-2-methylimidazo[1,2-a]pyridin-6-yl}-4-[3-methyl-3-(methylamino) pyrrolidin-1-yl]indazole-7-carboxamide (23 mg, 28%) as a solid. LCMS (ES, m/z): 450 [M+H]^{+ 1}H NMR (400 MHz, DMSO-d₆) δ 11.05 (s, 1H), 9.20 (d, J=1.6 Hz, 1H), 8.84 (s, 1H), 7.96-7.86 (m, 2H), 7.31-7.23 (m, 1H), 6.00 (d, J=8.4 Hz, 1H), 4.57 (q, J=7.3 Hz, 2H), 3.78 (d, J=8.4 Hz, 1H), 3.68 (m, 1H), 3.57 (d, J=10.3 Hz, 1H), 3.45 (d, J=10.2 Hz, 1H), 2.35 (s, 3H), 2.28 (s, 3H), 2.06 (dt, J=12.3, 6.4 Hz, 1H), 1.93-1.84 (m, 1H), 1.82 (m, 1H), 1.61 (t, J=7.2 Hz, 3H), 1.27 (s, 3H).

### Synthesis of Compound 547

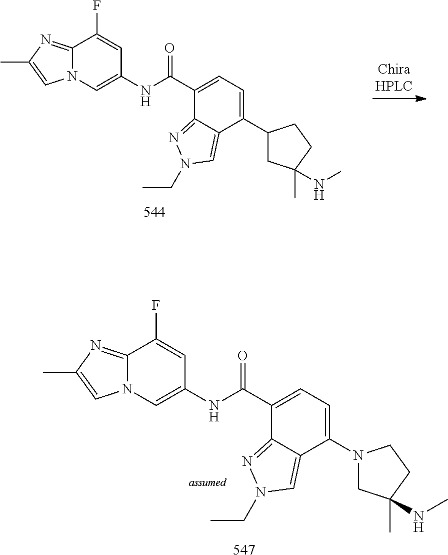

17 mg of 2-ethyl-N-{8-fluoro-2-methylimidazo[1,2-a]pyridin-6-yl}-4-[3-methyl-3-(methylamino) pyrrolidin-1-yl]indazole-7-carboxamide was purified by chiral-prep-HPLC (Condition 10, Gradient 1) to afford 2-ethyl-N-{8-fluoro-2-methylimidazo[1,2-a]pyridin-6-yl}-4-[(3R)-3-methyl-3-(methylamino)pyrrolidin-1-yl]indazole-7-carboxamide (6 mg, 33%) as a solid. LCMS (ES, m/z): 450 [M+H]^{+ 1}H NMR (400 MHz, DMSO-d₆) δ 11.05 (s, 1H), 9.20 (d, J=1.6 Hz, 1H), 8.84 (s, 1H), 7.96-7.86 (m, 2H), 7.31-7.23 (m, 1H), 6.00 (d, J=8.4 Hz, 1H), 4.57 (q, J=7.3 Hz, 2H), 3.78 (d, J=8.4 Hz, 1H), 3.68-3.67 (m, 1H), 3.57 (d, J=10.3 Hz, 1H), 3.45 (d, J=10.2 Hz, 1H), 2.35 (s, 3H), 2.28 (s, 3H), 2.06 (dt, J=12.3, 6.4 Hz, 1H), 1.93-1.84 (m, 1H), 1.82-1.81 (m, 1H), 1.61 (t, J=7.2 Hz, 3H), 1.27 (s, 3H).

### Synthesis of Compound 548

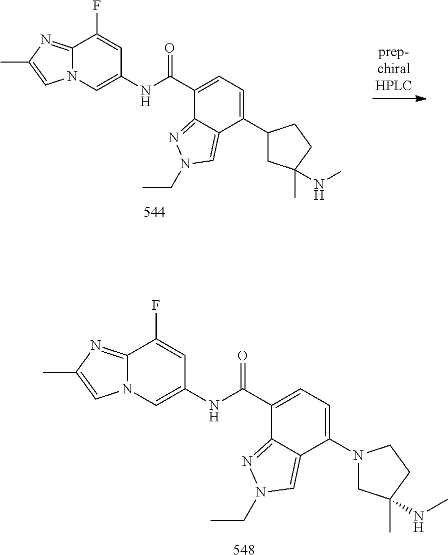

17 mg of 2-ethyl-N-{8-fluoro-2-methylimidazo[1,2-a]pyridin-6-yl}-4-[3-methyl-3-(methylamino) pyrrolidin-1-yl]indazole-7-carboxamide was purified by prep-chiral HPLC (Condition 10, Gradient 1) to afford 2-ethyl-N-{8-fluoro-2-methylimidazo [1,2-a]pyridin-6-yl}-4-[(3S)-3-methyl-3-(methylamino) pyrrolidin-1-yl]indazole-7-carboxamide (6 mg, 33%) as a solid.

LCMS (ES, m/z): 450 [M+H]^{+ 1}H NMR (400 MHz, DMSO-d₆) δ 11.05 (s, 1H), 9.20 (d, J=1.6 Hz, 1H), 8.84 (s, 1H), 7.96-7.86 (m, 2H), 7.31-7.23 (m, 1H), 6.00 (d, J=8.4 Hz, 1H), 4.57 (q, J=7.3 Hz, 2H), 3.78 (d, J=8.4 Hz, 1H), 3.68-3.67 (m, 1H), 3.57 (d, J=10.3 Hz, 1H), 3.45 (d, J=10.2 Hz, 1H), 2.35 (s, 3H), 2.28 (s, 3H), 2.06 (dt, J=12.3, 6.4 Hz, 1H), 1.93-1.84 (m, 1H), 1.82-1.81 (m, 1H), 1.61 (t, J=7.2 Hz, 3H), 1.27 (s, 3H).

### Example 268: Synthesis of Compound 545

### Synthesis of Intermediate C454

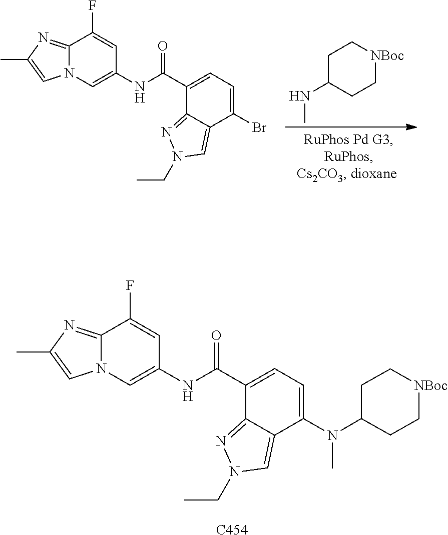

To a stirred solution of 4-bromo-2-ethyl-N-{8-fluoro-2-methylimidazo[1,2-a]pyridin-6-yl}indazole-7-carboxamide (150 mg, 0.36 mmol, 1 equiv), Cs₂CO₃ (235 mg, 0.72 mmol, 2 equiv) and tert-butyl 4-(methylamino)piperidine-1-carboxylate (116 mg, 0.54 mmol, 1.5 equiv) in dioxane (5 mL) were added RuPhos (34 mg, 0.072 mmol, 0.2 equiv) and RuPhos Palladacycle Gen.3 (30 mg, 0.036 mmol, 0.1 equiv) at room temperature under nitrogen atmosphere. The resulting mixture was stirred for 3 h at 100° C. under nitrogen atmosphere. The mixture was allowed to cool down to room temperature. The resulting mixture was concentrated under reduced pressure. The residue was purified by silica gel column chromatography, eluted with PE/EA (1:5) to afford tert-butyl 4-{[2-ethyl-7-({8-fluoro-2-methylimidazo[1,2-a]pyridin-6-yl}carbamoyl) indazol-4-yl](methyl)amino}piperidine-1-carboxylate (60 mg, 30%) as a solid. LCMS (ES, m/z): 550 [M+H]⁺

### Synthesis of Compound 545

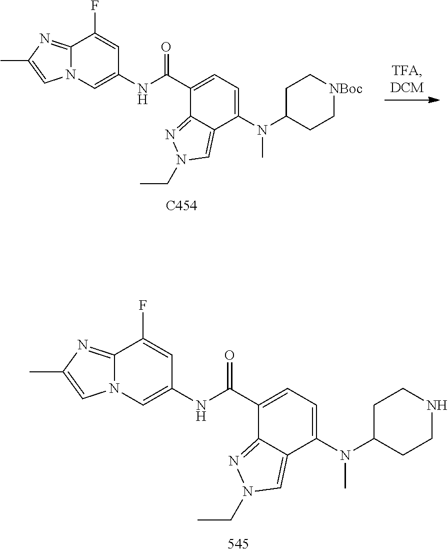

A solution of tert-butyl 4-{[2-ethyl-7-({8-fluoro-2-methylimidazo[1,2-a]pyridin-6-yl}carbamoyl) indazol-4-yl](methyl)amino}piperidine-1-carboxylate (60 mg, 0.109 mmol, 1 equiv) in TFA (2 mL) and DCM (2 mL) was stirred for 2 h at room temperature. The resulting mixture was concentrated under vacuum. The residue was basified to pH 8 with 7M NH₃(g) in MeOH. The resulting mixture was concentrated under reduced pressure. The residue was purified by reverse flash chromatography (Condition 3, Gradient 3) to afford 2-ethyl-N-(8-fluoro-2-methylimidazo[1,2-a]pyridin-6-yl)-4-(methyl(piperidin-4-yl)amino)-2H-indazole-7-carboxamide (16 mg, 33%) as a solid. LCMS (ES, m/z): 450 [M+H]^{+ 1}H NMR (300 MHz, DMSO-d₆) δ 11.11 (s, 1H), 9.21 (d, J=1.6 Hz, 1H), 8.70 (s, 1H), 7.99-7.86 (m, 2H), 7.33-7.23 (m, 1H), 6.34 (d, J=8.5 Hz, 1H), 4.60 (q, J=7.2 Hz, 2H), 3.99 (s, 1H), 3.06 (d, J=15.7 Hz, 5H), 2.67 (s, 2H), 2.35 (s, 3H), 1.72 (s, 4H), 1.62 (t, J=7.2 Hz, 3H).

### Example 269: Synthesis of Compound 550

### Synthesis of Intermediate C455

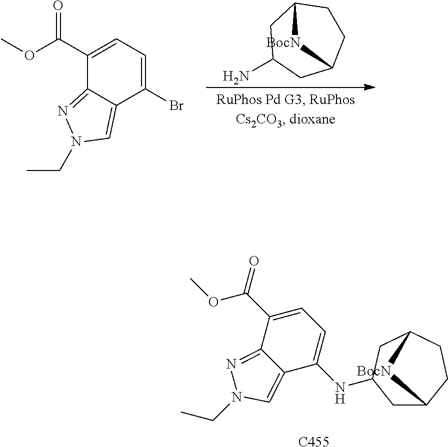 [SMILES: CCn1cc2c(Br)ccc(C(=O)OC)c2n1 ; CC(C)(C)OC(=O)N1[C@@H]2CC[C@H]1CC(N)C2 ; CCn1cc2c(NC3C[C@H]4CC[C@@H](C3)N4C(=O)OC(C)(C)C)ccc(C(=O)OC)c2n1 ; "C455"]

To a solution of methyl 4-bromo-2-ethylindazole-7-carboxylate (1 g, 3.532 mmol, 1 equiv) and tert-butyl (1R,5S)-3-amino-8-azabicyclo[3.2.1]octane-8-carboxylate (0.96 g, 4.238 mmol, 1.2 equiv) in dioxane (18 mL) were added RuPhos (0.33 g, 0.706 mmol, 0.2 equiv), Cs₂CO₃ (3.45 g, 10.596 mmol, 3.0 equiv) and RuPhos Palladacycle Gen.3 (0.3 g, 0.353 mmol, 0.1 equiv). After stirring for 3 h at 80° C. under a nitrogen atmosphere, the resulting mixture was concentrated under reduced pressure. The residue was purified by reverse flash chromatography w (Condition 5, Gradient 2) to afford methyl 4-{[(1R,5S)-8-(tert-butoxycarbonyl)-8-azabicyclo[3.2.1]octan-3-yl]amino}-2-ethylindazole-7-carboxylate (1.4 g, 93%) as a solid. LCMS (ES, m/z): 429 [M+H]⁺

### Synthesis of Intermediate C456

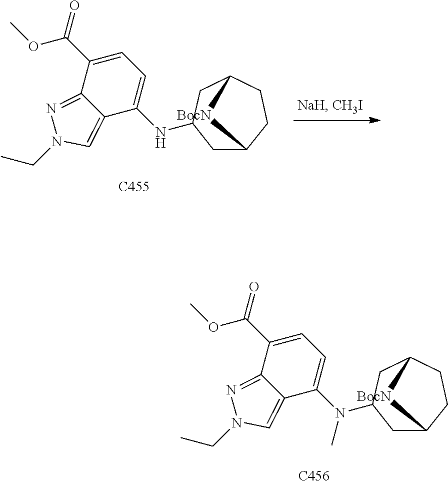

To a stirred solution of methyl 4-{[(1R,5S)-8-(tert-butoxycarbonyl)-8-azabicyclo[3.2.1]octan-3-yl]amino}-2-ethylindazole-7-carboxylate (500 mg, 1.167 mmol, 1 equiv) in DMF (6 mL) was added NaH (93.33 mg, 2.334 mmol, 2.0 equiv, 60%) in portions at 0° C. The resulting mixture was stirred for 0.5 h at 0° C. To the above mixture was added CH₃I (165.6 mg, 1.167 mmol, 1.0 equiv) dropwise at 0° C. The resulting mixture was stirred for additional 2 h at room temperature. The reaction mixture was quenched by the addition of MeOH (5 mL) at room temperature. The resulting mixture was concentrated under reduced pressure. The residue was purified by reverse flash chromatography (Condition 5, Gradient 2) to afford methyl 4-{[(1R,5S)-8-(tert-butoxycarbonyl)-8-azabicyclo[3.2.1]octan-3-yl](methyl)amino}-2-ethylindazole-7-carboxylate (500 mg, 97%) as a solid. LCMS (ES, m/z): 443 [M+H]⁺

### Synthesis of Intermediate C457

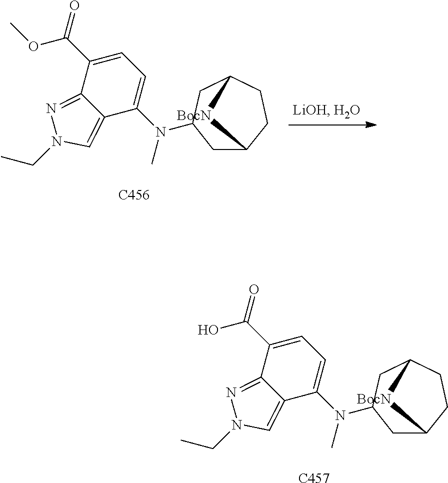

A solution of methyl 4-{[(1R,5S)-8-(tert-butoxycarbonyl)-8-azabicyclo[3.2.1]octan-3-yl](methyl)amino}-2-ethylindazole-7-carboxylate (500 mg, 1.130 mmol, 1 equiv) and LiOH·H₂O (162.3 mg, 6.780 mmol, 6.0 equiv) in MeOH (2 mL), H₂O (2 mL) and THF (2 mL) was stirred for 12 h at room temperature. The mixture was acidified to pH 2 with 2 mol/L aq. HCl. The precipitated solids were collected by filtration and washed with H₂O (3×5 mL) to afford 4-(((1R,5S)-8-(tert-butoxycarbonyl)-8-azabicyclo[3.2.1]octan-3-yl)(methyl)amino)-2-ethyl-2H-indazole-7-carboxylic acid (400 mg, 83%) as a solid. LCMS (ES, m/z): 429 [M+H]⁺

### Synthesis of Intermediate C458

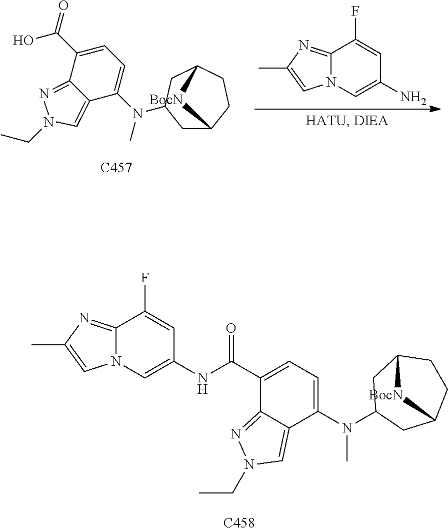

To a solution of 4-{[(1R,5S)-8-(tert-butoxycarbonyl)-8-azabicyclo[3.2.1]octan-3-yl](methyl)amino}-2-ethylindazole-7-carboxylic acid (100 mg, 0.233 mmol, 1 equiv) and TCFH (85.1 mg, 0.303 mmol, 1.3 equiv) in CH₃CN (2 mL) were added NMI (67 mg, 0.816 mmol, 3.5 equiv) and 8-fluoro-2-methylimidazo[1,2-a]pyridin-6-amine (46.2 mg, 0.28 mmol, 1.2 equiv) at room temperature. The resulting mixture was stirred for 1 h at room temperature. The resulting mixture was concentrated under reduced pressure. The residue was purified by reverse flash chromatography (Condition 5, Gradient 1) to afford tert-butyl (1R,5S)-3-{[2-ethyl-7-({8-fluoro-2-methylimidazo[1,2-a]pyridin-6-yl}carbamoyl) indazol-4-yl](methyl)amino}-8-azabicyclo[3.2.1]octane-8-carboxylate (55 mg, 41%) as a solid.

LCMS (ES, m/z): 576 [M+H]⁺

### Synthesis of Compound 550

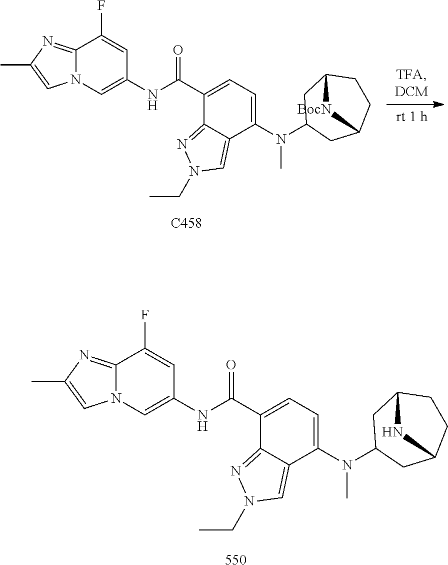

A solution of tert-butyl (1R,5S)-3-{[2-ethyl-7-({8-fluoro-2-methylimidazo[1,2-a]pyridin-6-yl}carbamoyl) indazol-4-yl](methyl)amino}-8-azabicyclo[3.2.1]octane-8-carboxylate (50 mg, 0.087 mmol, 1 equiv) and TFA (0.2 mL) in DCM (2 mL) was stirred for 1 h at room temperature. The mixture was basified to pH 8 with NH₃(g) in MeOH. The resulting mixture was concentrated under reduced pressure. The residue was purified by reverse flash chromatography (Condition 3, Gradient 2) to afford 4-[(1R,5S)-8-azabicyclo[3.2.1]octan-3-yl(methyl)amino]-2-ethyl-N-{8-fluoro-2-methylimidazo[1,2-a]pyridin-6-yl}indazole-7-carboxamide (20 mg, 48%) as a solid. LCMS (ES, m/z): 476 [M+H]^{+ 1}H NMR (300 MHz, DMSO-d₆) δ 11.09 (s, 1H), 9.22 (d, J=1.7 Hz, 1H), 8.69 (s, 1H), 7.99-7.86 (m, 2H), 7.28 (dd, J=12.4, 1.7 Hz, 1H), 6.37 (d, J=8.5 Hz, 1H), 4.60 (q, J=7.3 Hz, 2H), 4.38-4.18 (m, 1H), 3.52 (s, 2H), 3.07 (s, 3H), 2.35 (s, 3H), 1.87 (t, J=11.5 Hz, 2H), 1.74 (s, 3H), 1.63 (q, J=9.2, 7.3 Hz, 6H).

### Example 270: Synthesis of Compound 552

### Synthesis of Intermediate C459

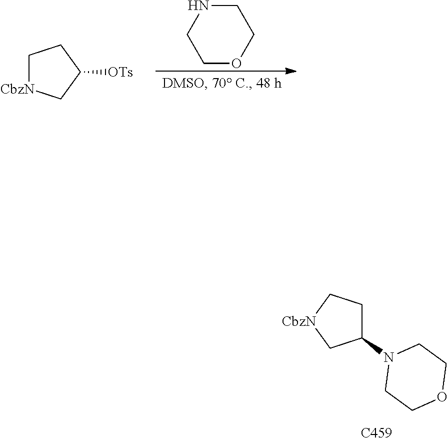

A solution of benzyl (3S)-3-[(4-methylbenzenesulfonyl)oxy]pyrrolidine-1-carboxylate (500 mg, 1.332 mmol, 1.0 equiv) in DMSO (5 mL) was treated with morpholine (580.1 mg, 6.66 mmol, 5.0 equiv) at room temperature. The resulting mixture was stirred for overnight at 70° C. under nitrogen atmosphere. The mixture was allowed to cool down to room temperature. The resulting mixture was extracted with EtOAc (3×30 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (1:1) to afford benzyl (3R)-3-(morpholin-4-yl)pyrrolidine-1-carboxylate (395 mg, 98%) as a solid. LCMS (ES, m/z): 291 [M+H]⁺

### Synthesis of Intermediate C460

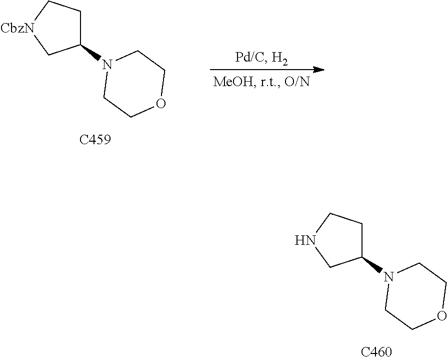

To a solution of benzyl (3R)-3-(morpholin-4-yl)pyrrolidine-1-carboxylate (395 mg, 1.360 mmol, 1.0 equiv) in 10 mL MeOH was added Pd/C (10%, 40 mg) in a pressure tank. The mixture was hydrogenated at room temperature under 5 psi of hydrogen pressure for overnight, filtered through a Celite pad and concentrated under reduced pressure to afford 4-[(3R)-pyrrolidin-3-yl]morpholine (170 mg, 80%) as a oil. ¹H NMR (400 MHz, Chloroform-d) δ 3.73 (t, J=4.8 Hz, 4H), 3.19-3.01 (m, 2H), 2.95 (dt, J=11.0, 7.5 Hz, 1H), 2.83-2.62 (m, 2H), 2.49 (ddt, J=27.6, 10.7, 4.0 Hz, 4H), 1.95 (dtd, J=12.3, 7.4, 4.7 Hz, 1H), 1.64 (dq, J=12.4, 7.7 Hz, 1H).

### Synthesis of Compound 552

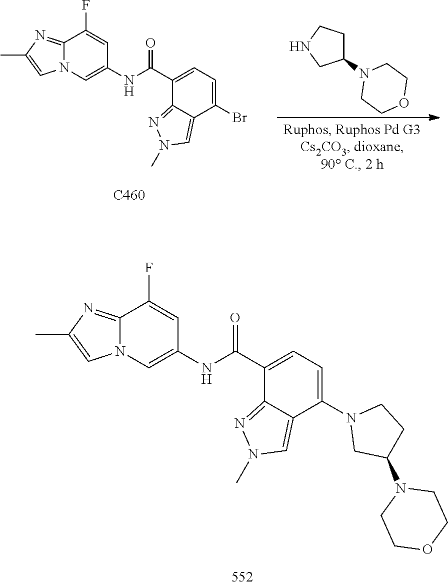 [SMILES: Cc1cn2cc(NC(=O)c3ccc(Br)c4cn(C)nc34)cc(F)c2n1 ; C1C[C@@H](N2CCOCC2)CN1 ; "C460" ; Cc1cn2cc(NC(=O)c3ccc(N4CC[C@@H](N5CCOCC5)C4)c4cn(C)nc34)cc(F)c2n1 ; "552"]

To a stirred mixture of 4-bromo-N-{8-fluoro-2-methylimidazo[1,2-a]pyridin-6-yl}-2-methylindazole-7-carboxamide (70 mg, 0.174 mmol, 1.0 equiv) and 4-[(3R)-pyrrolidin-3-yl]morpholine (32.6 mg, 0.209 mmol, 1.2 equiv) in dioxane (1 mL) were added Cs₂CO₃ (170.1 mg, 0.522 mmol, 3.0 equiv), Ruphos (8.1 mg, 0.017 mmol, 0.1 equiv) and RuPhos Palladacycle Gen.3 (14.5 mg, 0.017 mmol, 0.1 equiv) at room temperature. The resulting mixture was stirred for 3 h at 90° C. under nitrogen atmosphere. The mixture was allowed to cool down to room temperature. The crude product was purified by Prep-HPLC (Condition 10, Gradient 5) to afford N-{8-fluoro-2-methylimidazo[1,2-a]pyridin-6-yl}-2-methyl-4-[(3R)-3-(morpholin-4-yl)pyrrolidin-1-yl]indazole-7-carboxamide (40 mg, 48%) as a solid. LCMS (ES, m/z): 478 [M+H]^{+ 1}H NMR (400 MHz, DMSO-d₆) δ 11.01 (s, 1H), 9.19 (d, J=1.6 Hz, 1H), 8.86 (s, 1H), 7.93 (d, J=8.3 Hz, 1H), 7.90-7.86 (m, 1H), 7.30 (dd, J=12.5, 1.7 Hz, 1H), 6.04 (d, J=8.4 Hz, 1H), 4.27 (s, 3H), 3.96-3.84 (m, 1H), 3.75 (t, J=9.3 Hz, 1H), 3.64-3.61 (m, 5H), 3.49 (t, J=9.0 Hz, 1H), 3.00 (t, J=8.2 Hz, 1H), 2.53-2.52 (m, 4H), 2.34 (s, 3H), 2.33-2.18 (m, 1H), 1.90-1.88 (m, 1H).

### Example 271: Synthesis of Compound 554

### Synthesis of Intermediate C461

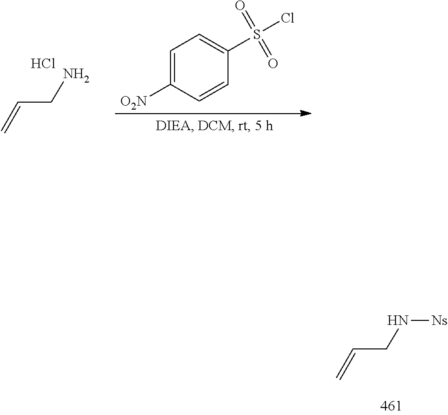

To a stirred solution of allylamine (2.0 g, 35.029 mmol, 1.0 equiv) and DIEA (9.0 g, 70.058 mmol, 2.0 equiv) in DCM (30 mL) was added 4-nitrobenzenesulfonyl chloride (7.7 g, 35.029 mmol, 1.0 equiv) dropwise at room temperature. The resulting mixture was stirred for 5 h at room temperature. The resulting mixture was extracted with CH₂Cl₂ (2×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (5:1) to afford 4-nitro-N-(prop-2-en-1-yl)benzenesulfonamide (5 g, 53%) as a solid. LCMS (ES, m/z): 243 [M+H]⁺

### Synthesis of Intermediate C462

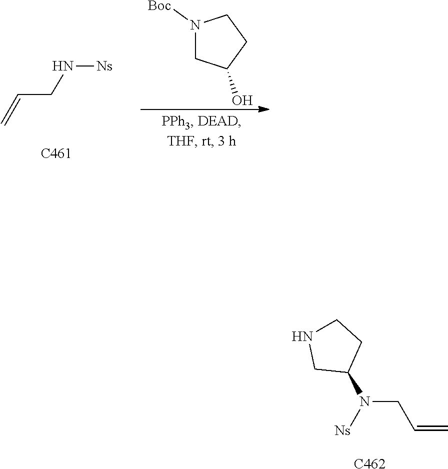

To a stirred solution of 4-nitro-N-(prop-2-en-1-yl)benzenesulfonamide (1.5 g, 6.192 mmol, 1.0 equiv), triphenylphosphine (2.7 g, 10.526 mmol, 1.7 equiv) and tert-butyl (3S)-3-hydroxypyrrolidine-1-carboxylate (1.7 g, 9.288 mmol, 1.5 equiv) in tetrahydrofuran (20 mL) was added DEAD (2.1 g, 12.384 mmol, 2.0 equiv) dropwise at 0° C. under nitrogen atmosphere. The resulting mixture was stirred for 3 h at room temperature under nitrogen atmosphere. The reaction was monitored by LCMS. The resulting mixture was diluted with deionized water (50 mL). The resulting mixture was extracted with EtOAc (2×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (5:1) to afford tert-butyl (3R)-3-[N-(prop-2-en-1-yl)4-nitrobenzenesulfonamido]pyrrolidine-1-carboxylate (1.6 g, 56%) as an oil. LCMS (ES, m/z): 412 [M+H]⁺

### Synthesis of Intermediate C463

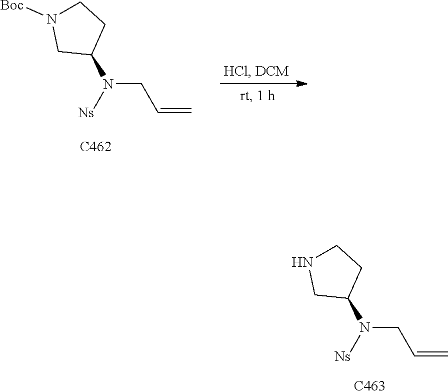

To a stirred solution of tert-butyl (3R)-3-[N-(prop-2-en-1-yl)4-nitrobenzenesulfonamido]pyrrolidine-1-carboxylate (800.0 mg, 1.944 mmol, 1.0 equiv) in DCM (3 mL) was added HCl (gas) in 1,4-dioxane (1 mL, 4 M) dropwise at room temperature. The resulting mixture was stirred for 1 h at room temperature. The resulting mixture was concentrated under reduced pressure. The residue was neutralized to PH=7 with NaHCO₃ aqueous and extracted with DCM (3×5 mL). The combined organic layers were dried by anhydrous Na₂SO₄ and filtered. The filtrate was concentrated in vacuo. This resulted in 4-nitro-N-(prop-2-en-1-yl)-N-[(3R)-pyrrolidin-3-yl] benzenesulfonamide (450 mg, 68%) as a solid. LCMS (ES, m/z): 312 [M+H]⁺

### Synthesis of Intermediate C464

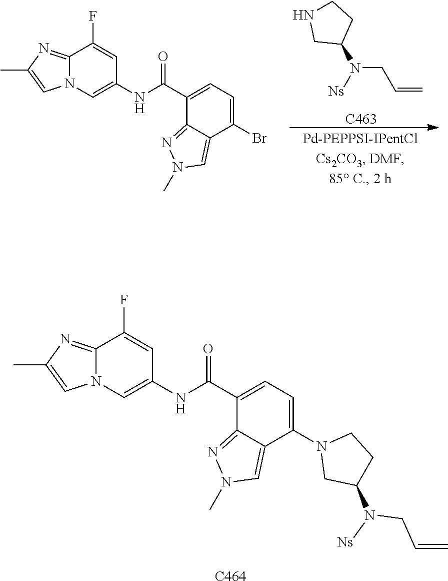

Into a 20 mL vial were added 4-bromo-N-{8-fluoro-2-methylimidazo[1,2-a]pyridin-6-yl}-2-methylindazole-7-carboxamide (200.0 mg, 0.497 mmol, 1.0 equiv), Cs₂CO₃ (325.0 mg, 0.994 mmol, 2.0 equiv), 4-nitro-N-(prop-2-en-1-yl)-N-[(3R)-pyrrolidin-3-yl]benzenesulfonamide (309.6 mg, 0.994 mmol, 2.0 equiv), Pd-PEPPSI-IPentCl (20.9 mg, 0.025 mmol, 0.05 equiv) and dimethylformamide (5 mL) at room temperature under nitrogen atmosphere. The resulting mixture was stirred for overnight at 70° C. under nitrogen atmosphere. The resulting mixture was concentrated under reduced pressure. The residue was purified by silica gel column chromatography, eluted with PE/EA (1:2) to afford N-{8-fluoro-2-methylimidazo[1,2-a]pyridin-6-yl}-2-methyl-4-[(3R)-3-[N-(prop-2-en-1-yl)4-nitrobenzenesulfonamido]pyrrolidin-1-yl]indazole-7-carboxamide (220 mg, 35%) as a solid. LCMS (ES, m/z): 633 [M+H]⁺

### Synthesis of Compound 554

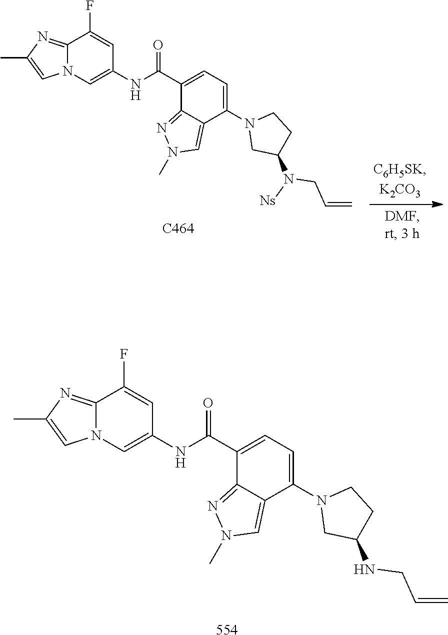

Into a 8 mL vial were added N-{8-fluoro-2-methylimidazo[1,2-a]pyridin-6-yl}-2-methyl-4-[(3R)-3-[N-(prop-2-en-1-yl)4-nitrobenzenesulfonamido]pyrrolidin-1-yl]indazole-7-carboxamide (100.0 mg, 0.158 mmol, 1.0 equiv), K₂CO₃ (43.6 mg, 0.316 mmol, 2.0 equiv) and C₆H₅SK (35.0 mg, 0.237 mmol, 1.5 equiv) at room temperature. The resulting mixture was stirred for 3 h at room temperature. The residue was purified by silica gel column chromatography, eluted with CH₂Cl₂/MeOH (10:1) to afford N-{8-fluoro-2-methylimidazo[1,2-a]pyridin-6-yl}-2-methyl-4-[(3R)-3-(prop-2-en-1-ylamino)pyrrolidin-1-yl]indazole-7-carboxamide (70 mg) as a solid. The crude product (70 mg) was purified by Prep-HPLC (Condition 13, Gradient 1) to afford N-{8-fluoro-2-methylimidazo [1,2-a]pyridin-6-yl}-2-methyl-4-[(3R)-3-(prop-2-en-1-ylamino)pyrrolidin-1-yl]indazole-7-carboxamide (15 mg, 21%) as a solid. LCMS (ES, m/z): 448 [M+H]^{+ 1}H NMR (400 MHz, DMSO-d₆) δ 11.01 (s, 1H), 9.20 (d, J=1.6 Hz, 1H), 8.83 (s, 1H), 7.96-7.86 (m, 2H), 7.31 (dd, J=12.4, 1.6 Hz, 1H), 6.02 (d, J=8.4 Hz, 1H), 5.89 (ddt, J=16.3, 11.3, 5.8 Hz, 1H), 5.21 (dq, J=17.3, 1.7 Hz, 1H), 5.07 (dt, J=10.3, 1.7 Hz, 1H), 4.27 (s, 3H), 3.78 (dd, J=19.9, 6.9 Hz, 2H), 3.64 (d, J=8.1 Hz, 1H), 3.51-3.40 (m, 2H), 3.26 (dt, J=5.8, 1.6 Hz, 2H), 2.37-2.33 (m, 3H), 2.17 (dd, J=11.9, 5.7 Hz, 1H), 1.93 (dd, J=12.2, 6.2 Hz, 1H).

### Example 272: Synthesis of Compound 556

### Synthesis of Intermediate C465

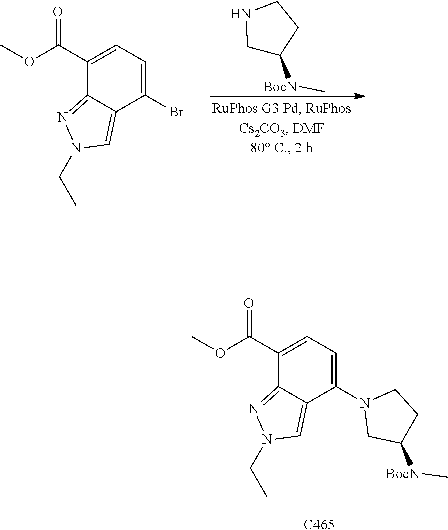

To a stirred mixture of methyl 4-bromo-2-ethylindazole-7-carboxylate (1.5 g, 5.298 mmol, 1 equiv) and tert-butyl N-methyl-N-[(3R)-pyrrolidin-3-yl]carbamate (1.27 g, 6.358 mmol, 1.2 equiv) in Dioxane (30 mL) were added Cs₂CO₃ (5.18 g, 15.894 mmol, 3 equiv) and RuPhos (0.49 g, 1.060 mmol, 0.2 equiv) and RuPhos Palladacycle Gen.3 (443.1 mg, 0.530 mmol, 0.1 equiv) at room temperature under nitrogen atmosphere. The resulting mixture was stirred for 2 h at 80° C. under nitrogen atmosphere. The resulting mixture was diluted with water (30 mL). The resulting mixture was extracted with EtOAc (3×30 mL). The combined organic layers were washed with water (3×30 mL), dried over anhydrous Na₂SO₄. After filtration, the filtrate was concentrated under reduced pressure. The residue was purified by silica gel column chromatography, eluted with EA to afford methyl 4-[(3R)-3-[(tert-butoxycarbonyl)(methyl)amino]pyrrolidin-1-yl]-2-ethylindazole-7-carboxylate (1.9 g, 89%) as a solid. LCMS (ES, m/z): 403 [M+H]⁺

### Synthesis of Intermediate C466

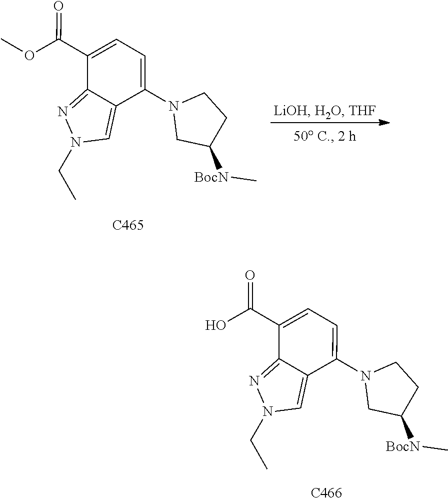

To a stirred mixture of methyl 4-[(3R)-3-[(tert-butoxycarbonyl)(methyl)amino]pyrrolidin-1-yl]-2-ethylindazole-7-carboxylate (1.9 g, 4.721 mmol, 1 equiv) in THF (20 mL) and H₂O (20 mL) was added LiOH·H₂O (0.99 g, 23.605 mmol, 5 equiv) at room temperature. The resulting mixture was stirred for 2 h at 50° C. The mixture was acidified to pH 4 with HCl (2M). The resulting mixture was extracted with EtOAc (3×40 mL). The combined organic layers were washed with water (3×40 mL), dried over anhydrous Na₂SO₄. After filtration, the filtrate was concentrated under reduced pressure to afford 4-[(3R)-3-[(tert-butoxycarbonyl)(methyl)amino]pyrrolidin-1-yl]-2-ethylindazole-7-carboxylic acid (1.4 g, 76%) as a solid. LCMS (ES, m/z): 389 [M+H]⁺

### Synthesis of Intermediate C467

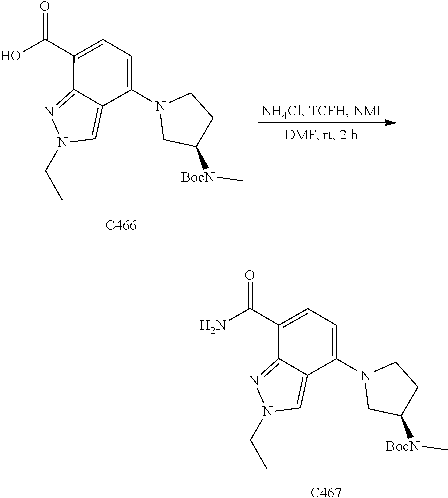

To a stirred mixture of 4-[(3R)-3-[(tert-butoxycarbonyl)(methyl)amino]pyrrolidin-1-yl]-2-ethylindazole-7-carboxylic acid (1.2 g, 3.089 mmol, 1 equiv) and NH₄Cl (0.83 g, 15.445 mmol, 5 equiv) in DMF (24 mL) were added NMI (1.01 g, 12.356 mmol, 4 equiv) and TCFH (1.73 g, 6.178 mmol, 2 equiv) at room temperature. The resulting mixture was stirred for 2 h at room temperature. The resulting mixture was filtered and the filter cake was washed with EtOAc (3×10 mL). The filtrate was concentrated under reduced pressure to afford tert-butyl N-[(3R)-1-(7-carbamoyl-2-ethylindazol-4-yl)pyrrolidin-3-yl]-N-methylcarbamate (900 mg, 75%) as a solid. LCMS (ES, m/z): 388 [M+H]⁺

### Synthesis of Intermediate C468

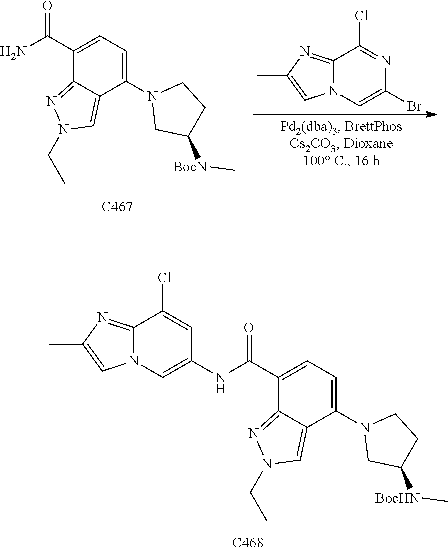

To a stirred solution of tert-butyl N-[(3R)-1-(7-carbamoyl-2-ethylindazol-4-yl)pyrrolidin-3-yl]-N-methylcarbamate (400 mg, 1.032 mmol, 1 equiv) and 6-bromo-8-chloro-2-methylimidazo[1,2-a]pyridine (380.1 mg, 1.548 mmol, 1.5 equiv) in Dioxane (8 mL) were added Cs₂CO₃ (672.7 mg, 2.064 mmol, 2 equiv) and BrettPhos (110.8 mg, 0.206 mmol, 0.2 equiv) and Pd₂(dba)₃ (94.5 mg, 0.103 mmol, 0.1 equiv) at room temperature under nitrogen atmosphere. The resulting mixture was stirred for 16 h at 100° C. under nitrogen atmosphere. The resulting mixture was diluted with water (10 mL). The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-[(3R)-1-[7-({8-chloro-2-methylimidazo[1,2-a]pyridin-6-yl}carbamoyl)-2-ethylindazol-4-yl]pyrrolidin-3-yl]-N-methylcarbamate (80 mg, 14%) as a solid. LCMS (ES, m/z): 552 [M+H]⁺

### Synthesis of Compound 556

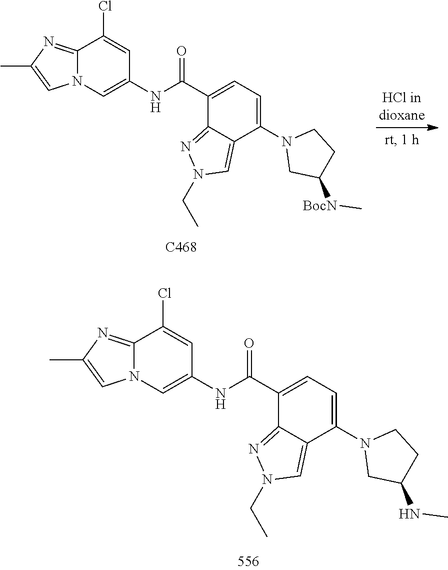

To a stirred mixture of tert-butyl N-[(3R)-1-[7-({8-chloro-2-methylimidazo[1,2-a]pyridin-6-yl}carbamoyl)-2-ethylindazol-4-yl]pyrrolidin-3-yl]-N-methylcarbamate (80 mg, 0.145 mmol, 1 equiv) in DCM (1.5 mL) was added HCl(gas) in 1,4-dioxane (0.5 mL, 4M) dropwise at room temperature. The resulting mixture was stirred for 1 h at room temperature. The resulting mixture was concentrated under vacuum. The crude product was purified by Prep-HPLC (Condition 10, Gradient 2) to afford N-{8-chloro-2-methylimidazo[1,2-a]pyridin-6-yl}-2-ethyl-4-[(3R)-3-(methylamino)pyrrolidin-1-yl]indazole-7-carboxamide (12.8 mg, 20%) as a solid. LCMS (ES, m/z): 452 [M+H]^{+ 1}H NMR (400 MHz, DMSO-d₆) δ 11.03 (s, 1H), 9.30 (d, J=1.8 Hz, 1H), 8.84 (s, 1H), 7.93 (d, J=8.2 Hz, 1H), 7.88 (s, 1H), 7.50 (d, J=1.7 Hz, 1H), 6.02 (d, J=8.3 Hz, 1H), 4.58 (q, J=7.2 Hz, 2H), 3.77 (dd, J=13.5, 7.1 Hz, 2H), 3.66 (s, 1H), 3.43 (dd, J=11.1, 3.9 Hz, 1H), 2.35 (d, J=3.5 Hz, 6H), 2.15 (dd, J=12.7, 6.1 Hz, 1H), 1.92 (dd, J=12.5, 6.7 Hz, 2H), 1.61 (t, J=7.2 Hz, 3H).

### Example 273: Synthesis of Compound 557

### Synthesis of Intermediate C469

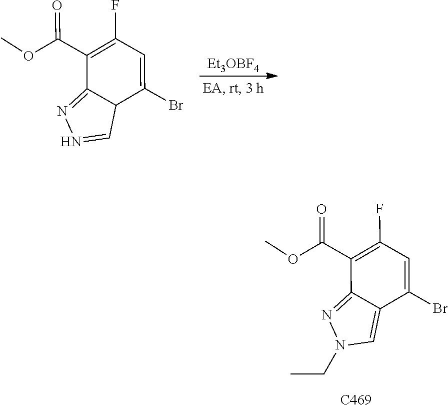

To a stirred solution of methyl 4-bromo-6-fluoro-2H-indazole-7-carboxylate (2 g, 7.324 mmol, 1 equiv) in EA (20 mL) was added tetrafluoroboranuide; triethyloxidanium (2.8 g, 14.648 mmol, 2 equiv) in portions at room temperature. The resulting mixture was stirred for 3 h at room temperature. The reaction was quenched by the addition of saturated aqueous NaHCO₃ (100 mL) at room temperature. The resulting mixture was extracted with EtOAc (3×20 mL). The combined organic layers were washed with brine (1×50 mL), dried over anhydrous Na₂SO₄. After filtration, the filtrate was concentrated under reduced pressure to afford methyl 4-bromo-2-ethyl-6-fluoroindazole-7-carboxylate (2.1 g, 95%) as a solid. LCMS (ES, m/z): 301 [M+H]⁺

### Synthesis of Intermediate C470

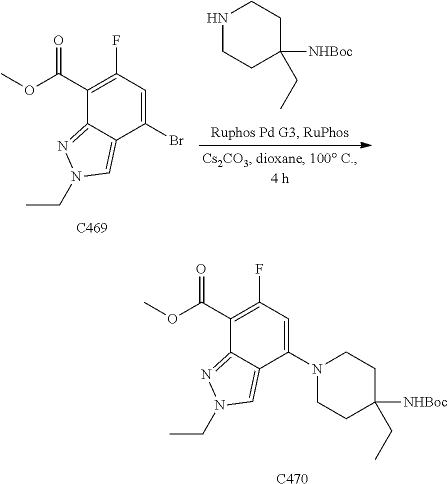

To a stirred solution of methyl 4-bromo-2-ethyl-6-fluoroindazole-7-carboxylate (300 mg, 0.996 mmol, 1 equiv) and tert-butyl N-(4-ethylpiperidin-4-yl)carbamate (455 mg, 1.992 mmol, 2 equiv) in dioxane (10 mL) were added Cs₂CO₃ (649 mg, 1.992 mmol, 2 equiv), RuPhos (93 mg, 0.199 mmol, 0.2 equiv) and RuPhos Palladacycle Gen.3 (83 mg, 0.1 mmol, 0.1 equiv) at room temperature under nitrogen atmosphere. The resulting mixture was stirred for 5 h at 100° C. under nitrogen atmosphere. The mixture was allowed to cool down to room temperature. The resulting mixture was concentrated under vacuum. The residue was purified by silica gel column chromatography, eluted with PE/EA (1:1) to afford methyl 4-{4-[(tert-butoxycarbonyl)amino]-4-ethylpiperidin-1-yl}-2-ethyl-6-fluoroindazole-7-carboxylate (150 mg, 34%) as a solid. LCMS (ES, m/z): 449 [M+H]⁺

### Synthesis of Intermediate C471

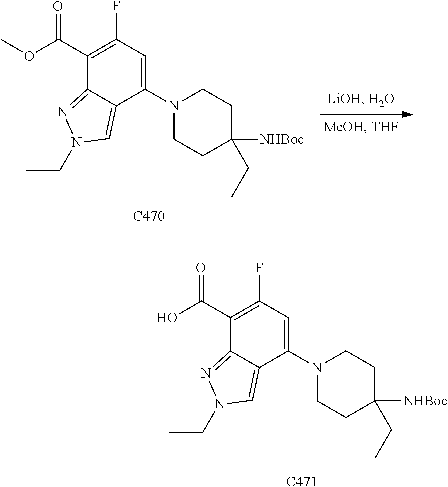 [SMILES: CCn1cc2c(N3CCC(CC)(NC(=O)OC(C)(C)C)CC3)cc(F)c(C(=O)OC)c2n1 ; "C470" ; CCn1cc2c(N3CCC(CC)(NC(=O)OC(C)(C)C)CC3)cc(F)c(C(=O)O)c2n1 ; "C471"]

A mixture of methyl 4-{4-[(tert-butoxycarbonyl)amino]-4-ethylpiperidin-1-yl}-2-ethyl-6-fluoroindazole-7-carboxylate (376 mg, 0.838 mmol, 1 equiv) and LiOH (201 mg, 8.38 mmol, 10 equiv) in H₂O (4 mL), THF (4 mL) and MeOH (4 mL) was stirred for overnight at room temperature. The resulting mixture was concentrated under vacuum. The resulting mixture was diluted with water (20 mL). The mixture was acidified to pH 2 with 1 M HCl (aq.). The resulting mixture was extracted with CH₂Cl₂ (3×5 mL). The combined organic layers were washed with brine (1×10 mL), dried over anhydrous Na₂SO₄. After filtration, the filtrate was concentrated under reduced pressure to afford 4-{4-[(tert-butoxycarbonyl)amino]-4-ethylpiperidin-1-yl}-2-ethyl-6-fluoroindazole-7-carboxylic acid (136 mg, 37%) as a solid. LCMS (ES, m/z): 435 [M+H]⁺

### Synthesis of Intermediate C472

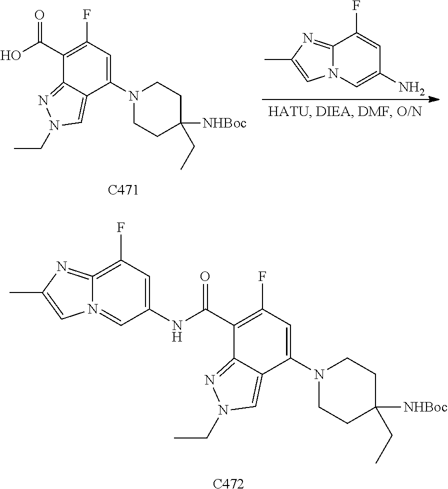

To a stirred solution of 4-{4-[(tert-butoxycarbonyl)amino]-4-ethylpiperidin-1-yl}-2-ethyl-6-fluoroindazole-7-carboxylic acid (136 mg, 0.313 mmol, 1 equiv) and 8-fluoro-2-methylimidazo[1,2-a]pyridin-6-amine (57 mg, 0.344 mmol, 1.1 equiv) in DMF (5 mL) were added HATU (143 mg, 0.376 mmol, 1.2 equiv) and DIEA (202 mg, 1.565 mmol, 5 equiv) at room temperature under nitrogen atmosphere. The resulting mixture was stirred for 24 h at room temperature. The reaction was quenched by the addition of water (20 mL) at room temperature. The resulting solids were collected by filtration and washed with water (3×5 mL) to afford tert-butyl N-{4-ethyl-1-[2-ethyl-6-fluoro-7-({8-fluoro-2-methylimidazo[1,2-a]pyridin-6-yl}carbamoyl) indazol-4-yl]piperidin-4-yl}carbamate (130 mg, 71%) as a solid. LCMS (ES, m/z): 582 [M+H]⁺

### Synthesis of Compound 557

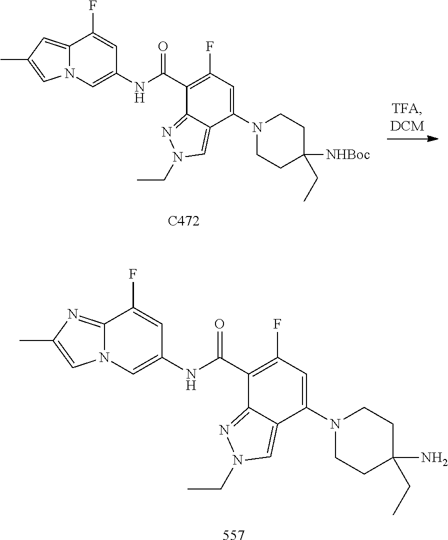

A mixture of tert-butyl N-{4-ethyl-1-[2-ethyl-6-fluoro-7-({8-fluoro-2-methylimidazo[1,2-a]pyridin-6-yl}carbamoyl) indazol-4-yl]piperidin-4-yl}carbamate (150 mg, 0.258 mmol, 1 equiv) in TFA (1 mL) and DCM (3 mL) was stirred for 2 h at room temperature. The resulting mixture was concentrated under reduced pressure. The residue was basified to pH 8 with 7M NH₃(g) in MeOH. The resulting mixture was concentrated under vacuum. The residue was purified by reverse flash chromatography (Condition 3, Gradient 2) to afford 4-(4-amino-4-ethylpiperidin-1-yl)-2-ethyl-6-fluoro-N-{8-fluoro-2-methylimidazo[1,2-a]pyridin-6-yl}indazole-7-carboxamide (40 mg, 32%) as a solid. LCMS (ES, m/z): 482 [M+H]^{+ 1}H NMR (400 MHz, DMSO-d₆) δ 10.97 (s, 1H), 9.19 (d, J=1.6 Hz, 1H), 8.78 (s, 1H), 7.89 (d, J=3.1 Hz, 1H), 7.21 (dd, J=12.4, 1.7 Hz, 1H), 6.22 (d, J=15.5 Hz, 1H), 4.50 (q, J=7.3 Hz, 2H), 3.63 (dt, J=13.1, 4.3 Hz, 2H), 3.51-3.40 (m, 2H), 2.35 (s, 3H), 1.66-1.58 (m, 1H), 1.57 (q, J=7.3, 6.2 Hz, 4H), 1.52-1.45 (m, 2H), 1.40 (q, J=7.4 Hz, 2H), 0.88 (t, J=7.4 Hz, 3H).

### Example 274: Synthesis of Compounds 559 and 560

### Synthesis of Intermediate C473

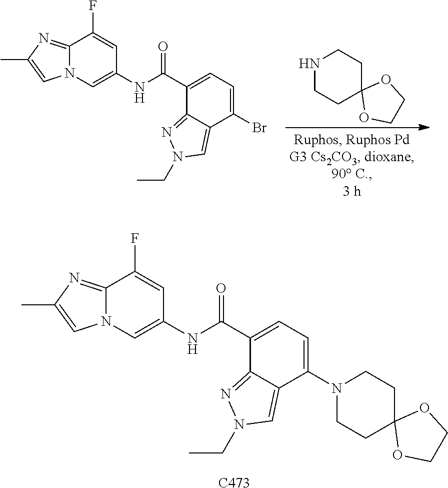

To a stirred solution of 4-bromo-2-ethyl-N-{8-fluoro-2-methylimidazo[1,2-a]pyridin-6-yl}indazole-7-carboxamide (500 mg, 1.201 mmol, 1.0 equiv) and 1,4-dioxa-8-azaspiro[4.5]decane (206.4 mg, 1.441 mmol, 1.2 equiv) in dioxane (5 mL) were added Cs₂CO₃ (1.27 g, 3.90 mmol, 3.0 equiv), Ruphos (112.1 mg, 0.240 mmol, 0.2 equiv) and RuPhos Palladacycle Gen.3 (100.4 mg, 0.120 mmol, 0.1 equiv) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with CH₂Cl₂/MeOH (10:1) to afford 4-{1,4-dioxa-8-azaspiro[4.5]decan-8-yl}-2-ethyl-N-{8-fluoro-2-methylimidazo[1,2-a]pyridin-6-yl}indazole-7-carboxamide (510 mg, 89%) as a solid. LCMS (ES, m/z): 479 [M+H]⁺

### Synthesis of Intermediate C474

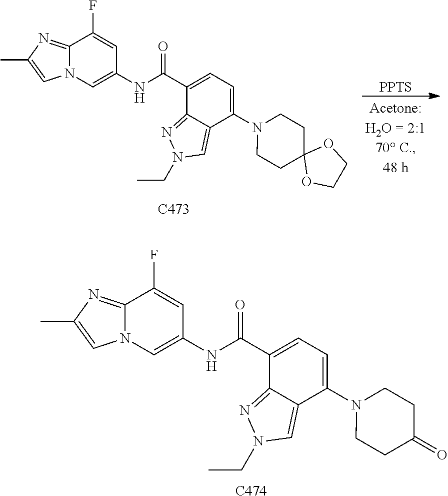

A solution of 4-{1,4-dioxa-8-azaspiro[4.5]decan-8-yl}-2-ethyl-N-{8-fluoro-2-methylimidazo[1,2-a]pyridin-6-yl}indazole-7-carboxamide (370 mg, 0.773 mmol, 1 equiv) in Acetone (4 mL) and H₂O (2 mL) was treated with PPTS (3.89 g, 15.460 mmol, 20 equiv) at room temperature. The resulting mixture was stirred for 48 h at 70° C. The mixture was allowed to cool down to room temperature. The mixture was neutralized to pH 7 with saturated NaHCO₃ (aq.). The resulting mixture was extracted with EtOAc (3×20 mL). The combined organic layers were washed with brine (1×10 mL), dried over anhydrous Na₂SO₄. After filtration, the filtrate was concentrated under reduced pressure to afford 2-ethyl-N-{8-fluoro-2-methylimidazo[1,2-a]pyridin-6-yl}-4-(4-oxopiperidin-1-yl) indazole-7-carboxamide (230 mg, 68.47%) as a yellow solid. LCMS (ES, m/z): 435 [M+H]⁺

### Synthesis of Compound 559

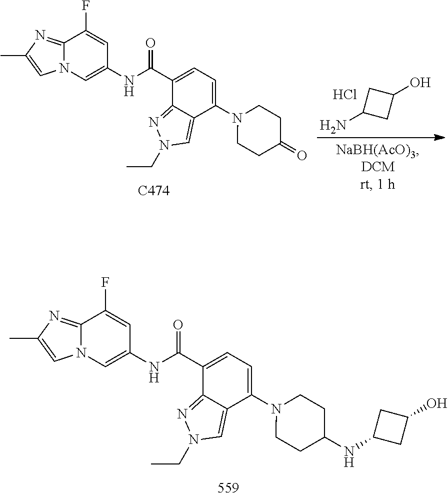

To a stirred mixture of 2-ethyl-N-{8-fluoro-2-methylimidazo[1,2-a]pyridin-6-yl}-4-(4-oxopiperidin-1-yl) indazole-7-carboxamide (100 mg, 0.230 mmol, 1 equiv) and 3-aminocyclobutan-1-ol (24.06 mg, 0.276 mmol, 1.2 equiv) in DCM (1 mL) was added NaBH(AcO)₃ (97.56 mg, 0.460 mmol, 2 equiv) in portions at room temperature under nitrogen atmosphere. The resulting mixture was stirred for 1 h at room temperature. The reaction was quenched with Water at room temperature. The resulting mixture was concentrated under reduced pressure. The crude product was purified by Prep-HPLC (Condition 10, Gradient 14) to afford 2-ethyl-N-{8-fluoro-2-methylimidazo[1,2-a]pyridin-6-yl}-4-(4-{[(1s,3s)-3-hydroxycyclobutyl]amino}piperidin-1-yl) indazole-7-carboxamide (4.1 mg, 4%) as a solid. LCMS (ES, m/z): 506 [M+H]^{+ 1}H NMR (300 MHz, Methanol-d₆) δ 9.04 (d, J=1.6 Hz, 1H), 8.47 (s, 1H), 8.03 (d, J=8.1 Hz, 1H), 7.68 (d, J=3.0 Hz, 1H), 7.16 (dd, J=11.7, 1.7 Hz, 1H), 6.48 (d, J=8.2 Hz, 1H), 4.59 (q, J=7.3 Hz, 2H), 4.40 (s, 1H), 3.97 (d, J=12.8 Hz, 2H), 3.68 (p, J=7.2 Hz, 1H), 3.00 (t, J=12.3 Hz, 2H), 2.73 (t, J=11.2 Hz, 1H), 2.41 (s, 3H), 2.18 (q, J=6.8 Hz, 4H), 2.01 (d, J=12.5 Hz, 2H), 1.69 (t, J=7.3 Hz, 3H), 1.57 (q, J=10.1 Hz, 2H).

### Synthesis of Compound 560

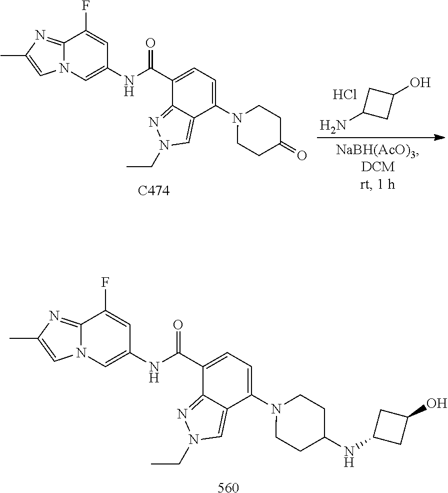

To a stirred mixture of 2-ethyl-N-{8-fluoro-2-methylimidazo[1,2-a]pyridin-6-yl}-4-(4-oxopiperidin-1-yl) indazole-7-carboxamide (100 mg, 0.230 mmol, 1 equiv) and 3-aminocyclobutan-1-ol (24.06 mg, 0.276 mmol, 1.2 equiv) in DCM (1 mL) was added NaBH(AcO)₃ (97.56 mg, 0.460 mmol, 2 equiv) in portions at room temperature under nitrogen atmosphere. The resulting mixture was stirred for 1 h at room temperature. The reaction was quenched with water at room temperature. The resulting mixture was concentrated under reduced pressure. The crude product was purified by Prep-HPLC (Condition 10, Gradient 14) to afford 2-ethyl-N-{8-fluoro-2-methylimidazo[1,2-a]pyridin-6-yl}-4-(4-{[(1r,3r)-3-hydroxycyclobutyl]amino}piperidin-1-yl) indazole-7-carboxamide (3.6 mg, 3%) as a solid. LCMS (ES, m/z): 506 [M+H]^{+ 1}H NMR (300 MHz, Methanol-d₆) δ 9.07 (d, J=1.7 Hz, 1H), 8.50 (s, 1H), 8.06 (d, J=8.1 Hz, 1H), 7.71 (dd, J=3.0, 1.0 Hz, 1H), 7.19 (dd, J=11.8, 1.6 Hz, 1H), 6.50 (d, J=8.2 Hz, 1H), 4.61 (q, J=7.3 Hz, 2H), 4.06-3.90 (m, 3H), 3.12-2.88 (m, 3H), 2.86-2.57 (m, 3H), 2.43 (d, J=0.8 Hz, 3H), 2.04 (d, J=12.6 Hz, 2H), 1.87-1.67 (m, 5H), 1.59 (q, J=12.1, 11.1 Hz, 2H).

### Example 275: Synthesis of Compound 562

### Synthesis of Intermediate C475

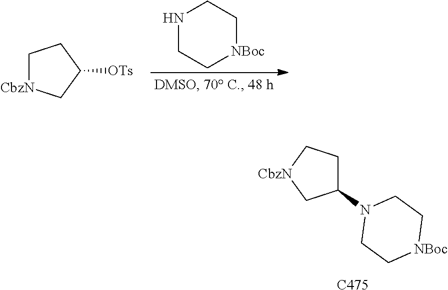

A solution of benzyl (3S)-3-[(4-methylbenzenesulfonyl)oxy]pyrrolidine-1-carboxylate (500 mg, 1.332 mmol, 1.0 equiv) in DMSO (5 mL) was treated with tert-butyl piperazine-1-carboxylate (1.24 g, 6.660 mmol, 5.0 equiv) at room temperature. The resulting mixture was stirred for overnight at 70° C. under nitrogen atmosphere. The mixture was allowed to cool down to room temperature. The resulting mixture was extracted with EtOAc (3×30 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (2:1) to afford tert-butyl 4-[(3R)-1-[(benzyloxy)carbonyl]pyrrolidin-3-yl]piperazine-1-carboxylate (510 mg, 98%) as an oil. LCMS (ES, m/z): 390 [M+H]⁺

### Synthesis of Intermediate C476

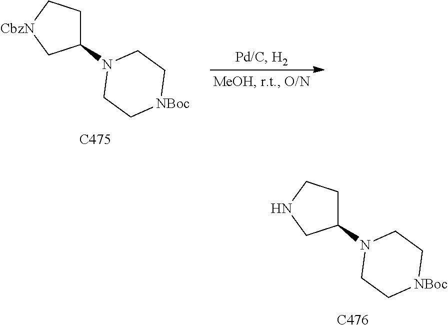

To a solution of tert-butyl 4-[(3R)-1-[(benzyloxy)carbonyl]pyrrolidin-3-yl]piperazine-1-carboxylate (510 mg, 1.309 mmol, 1.0 equiv) in 10 mL MeOH was added Pd/C (10%, 50 mg) in a pressure tank. The mixture was hydrogenated at room temperature under 5 psi of hydrogen pressure for overnight, filtered through a Celite pad and concentrated under reduced pressure to afford tert-butyl 4-[(3R)-pyrrolidin-3-yl]piperazine-1-carboxylate (350 mg, 105%) as an oil.

### Synthesis of Intermediate C477

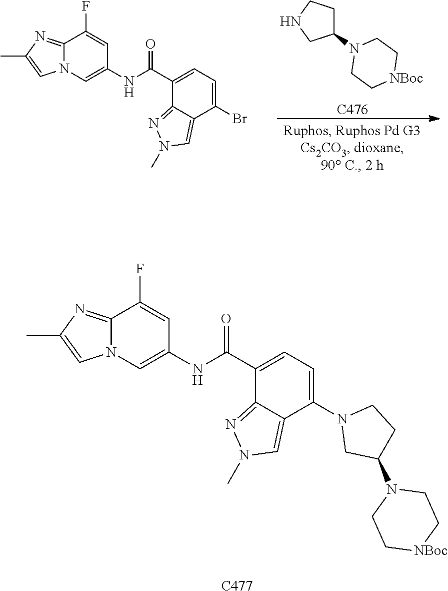

To a stirred mixture of 4-bromo-N-{8-fluoro-2-methylimidazo[1,2-a]pyridin-6-yl}-2-methylindazole-7-carboxamide (70 mg, 0.174 mmol, 1.0 equiv) and tert-butyl 4-[(3R)-pyrrolidin-3-yl]piperazine-1-carboxylate (53.3 mg, 0.209 mmol, 1.2 equiv) in dioxane (1 mL) were added Cs₂CO₃ (170.1 mg, 0.522 mmol, 3.0 equiv), Ruphos (8.1 mg, 0.017 mmol, 0.1 equiv) and RuPhos Palladacycle Gen.3 (14.6 mg, 0.017 mmol, 0.1 equiv) at room temperature. The resulting mixture was stirred for 3 h at 90° C. under nitrogen atmosphere. The mixture was allowed to cool down to room temperature. The residue was purified by silica gel column chromatography, eluted with CH₂Cl₂/MeOH (10:1) to afford tert-butyl 4-[(3R)-1-[7-({8-fluoro-2-methylimidazo[1,2-a]pyridin-6-yl}carbamoyl)-2-methylindazol-4-yl]pyrrolidin-3-yl]piperazine-1-carboxylate (94 mg, 94%) as a solid. LCMS (ES, m/z): 577 [M+H]⁺

### Synthesis of Compound 562

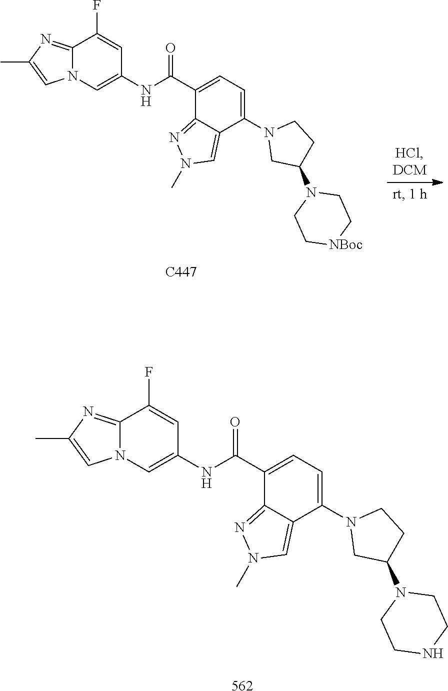 [SMILES: Cc1cn2cc(NC(=O)c3ccc(N4CC[C@@H](N5CCN(C(=O)OC(C)(C)C)CC5)C4)c4cn(C)nc34)cc(F)c2n1 ; "C447" ; Cc1cn2cc(NC(=O)c3ccc(N4CC[C@@H](N5CCNCC5)C4)c4cn(C)nc34)cc(F)c2n1 ; "562"]

A solution of tert-butyl 4-[(3R)-1-[7-({8-fluoro-2-methylimidazo[1,2-a]pyridin-6-yl}carbamoyl)-2-methylindazol-4-yl]pyrrolidin-3-yl]piperazine-1-carboxylate (93 mg, 0.161 mmol, 1.0 equiv) in DCM (0.9 mL) was treated with HCl (gas) in 1,4-dioxane (0.3 mL) at room temperature. The resulting mixture was stirred for 1 h at room temperature. The resulting mixture was concentrated under reduced pressure. The crude product was purified by Prep-HPLC (Condition 18, Gradient 1) to afford N-{8-fluoro-2-methylimidazo[1,2-a]pyridin-6-yl}-2-methyl-4-[(3R)-3-(piperazin-1-yl)pyrrolidin-1-yl]indazole-7-carboxamide hydrochloride (39 mg, 47%) as a solid. LCMS (ES, m/z): 477 [M+H]^{+ 1}H NMR (400 MHz, DMSO-d₆) δ 11.33 (s, 1H), 10.01 (s, 2H), 9.60 (s, 1H), 9.04 (s, 1H), 8.30 (s, 1H), 8.13 (d, J=11.7 Hz, 1H), 7.98 (d, J=8.2 Hz, 1H), 6.12 (d, J=8.4 Hz, 1H), 4.32 (s, 3H), 4.17 (d, J=5.7 Hz, 1H), 4.05-4.04 (m, 4H), 3.92-3.91 (m, 2H), 3.64-3.63 (m, 2H), 3.61-3.49 (m, 4H), 2.64-2.63 (m, 1H), 2.51 (s, 3H), 2.44-2.43 (m, 1H).

### Example 276: Synthesis of Compound 563

### Synthesis of Intermediate C448

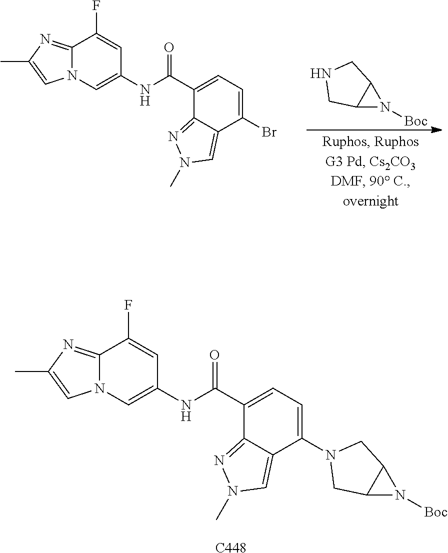

To a solution of 4-bromo-N-{8-fluoro-2-methylimidazo[1,2-a]pyridin-6-yl}-2-methylindazole-7-carboxamide (100.0 mg, 0.249 mmol, 1.0 equiv) and tert-butyl 3,6-diazabicyclo[3.1.0]hexane-6-carboxylate (68.7 mg, 0.373 mmol, 1.5 equiv) in DMF (4 mL) were added Cs₂CO₃ (162.0 mg, 0.498 mmol, 2.0 equiv) and Ruphos (11.6 mg, 0.025 mmol, 0.1 equiv), RuPhos Palladacycle Gen.3 (20.7 mg, 0.025 mmol, 0.1 equiv). After stirring for overnight at 90° C. under a nitrogen atmosphere, the resulting mixture was concentrated under reduced pressure. The residue was purified by silica gel column chromatography, eluted with PE/EA (1:3) to afford tert-butyl 3-[7-({8-fluoro-2-methylimidazo[1,2-a]pyridin-6-yl}carbamoyl)-2-methylindazol-4-yl]-3,6-diazabicyclo[3.1.0]hexane-6-carboxylate (100 mg, 72%) as a solid. LCMS (ES, m/z): 506 [M+H]⁺

### Synthesis of Compound 563

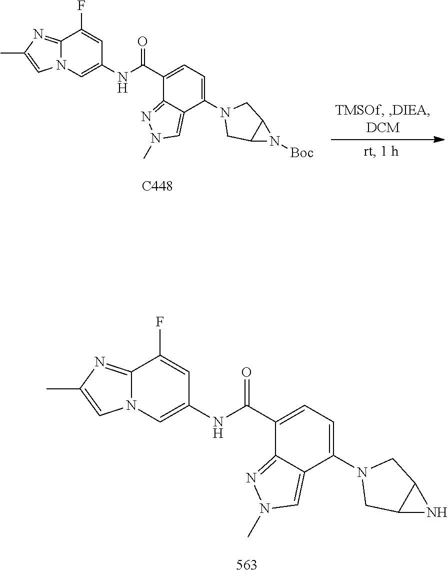

A solution of tert-butyl 3-[7-({8-fluoro-2-methylimidazo[1,2-a]pyridin-6-yl}carbamoyl)-2-methylindazol-4-yl]-3,6-diazabicyclo[3.1.0]hexane-6-carboxylate (100.0 mg, 0.198 mmol, 1.0 equiv) in DCM (2 mL) was treated with DIEA (76.7 mg, 0.594 mmol, 3.0 equiv), TMSOTf (131.8 mg, 0.594 mmol, 3.0 equiv) for 1 h at room temperature. The reaction was monitored by LCMS. The resulting mixture was concentrated under reduced pressure. The crude product was purified by Prep-HPLC (Condition 13, Gradient 1) to afford 4-{3,6-diazabicyclo[3.1.0]hexan-3-yl}-N-{8-fluoro-2-methylimidazo[1,2-a]pyridin-6-yl}-2-methylindazole-7-carboxamide (10 mg, 11%) as a solid. LCMS (ES, m/z): 406 [M+H]^{+ 1}H NMR (400 MHz, DMSO-d₆) δ 10.97 (s, 1H), 9.21 (d, J=1.7 Hz, 1H), 8.77 (s, 1H), 7.97 (d, J=7.7 Hz, 1H), 7.92 (d, J=3.1 Hz, 1H), 7.35 (dd, J=12.2, 1.7 Hz, 1H), 6.68 (d, J=7.8 Hz, 1H), 4.33 (s, 3H), 3.28-3.19 (m, 4H), 2.65 (d, J=12.8 Hz, 2H), 2.36 (s, 3H).

### Example 277: Synthesis of Compound 564

### Synthesis of Intermediate C449

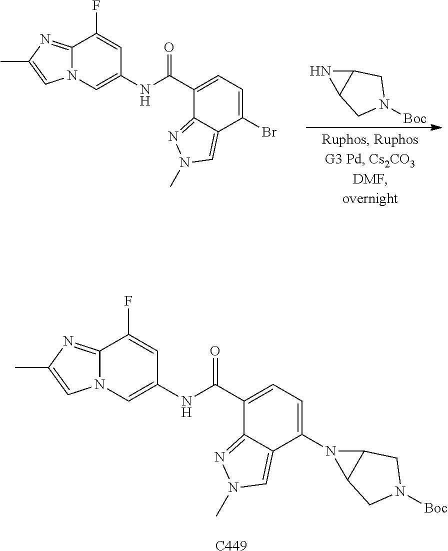

To a solution of 4-bromo-N-{8-fluoro-2-methylimidazo[1,2-a]pyridin-6-yl}-2-methylindazole-7-carboxamide (100.0 mg, 0.249 mmol, 1.0 equiv) and tert-butyl 3,6-diazabicyclo[3.1.0]hexane-3-carboxylate (68.7 mg, 0.373 mmol, 1.5 equiv) in DMF (4 mL) were added Cs₂CO₃ (162.0 mg, 0.498 mmol, 2.0 equiv) and Ruphos (11.60 mg, 0.025 mmol, 0.1 equiv), RuPhos Palladacycle Gen.3 (20.7 mg, 0.025 mmol, 0.1 equiv). After stirring for overnight at 90° C. under a nitrogen atmosphere, the resulting mixture was concentrated under reduced pressure. The residue was purified by silica gel column chromatography, eluted with PE/EA (1:3) to afford tert-butyl6-[7-({8-fluoro-2-methylimidazo[1,2-a]pyridin-6-yl}carbamoyl)-2-methylindazol-4-yl]-3,6-diazabicyclo[3.1.0]hexane-3-carboxylate (80 mg, 57%) as a solid. LCMS (ES, m/z): 506 [M+H]

### Synthesis of Compound 564

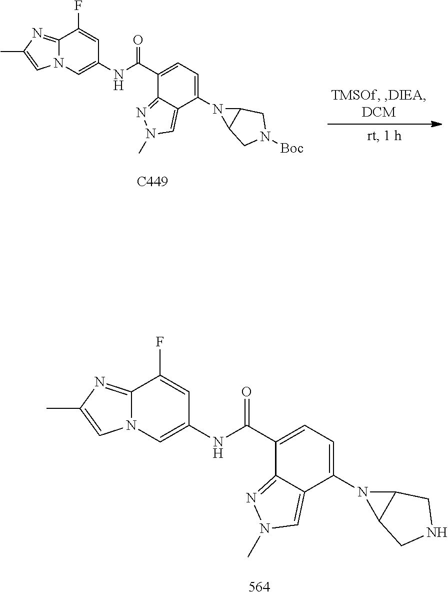

Into a 40 mL vial were added tert-butyl 6-[7-({8-fluoro-2-methylimidazo[1,2-a]pyridin-6-yl}carbamoyl)-2-methylindazol-4-yl]-3,6-diazabicyclo[3.1.0]hexane-3-carboxylate (80.0 mg, 0.158 mmol, 1.0 equiv), DCM (2 mL), DIEA (61.3 mg, 0.474 mmol, 3.0 equiv) and TMSOTf (105.5 mg, 0.474 mmol, 3.0 equiv) at room temperature. The resulting mixture was stirred for 1 h at room temperature. The resulting mixture was concentrated under reduced pressure. The crude product was purified by Prep-HPLC (Condition 13, Gradient 1) to afford 4-{3,6-diazabicyclo[3.1.0]hexan-6-yl}-N-{8-fluoro-2-methylimidazo[1,2-a]pyridin-6-yl}-2-methylindazole-7-carboxamide (10 mg, 15%) as a solid. LCMS (ES, m/z): 406 [M+H]^{+ 1}H NMR (400 MHz, DMSO-d₆) δ 10.97 (s, 1H), 9.20 (s, 1H), 8.77 (s, 1H), 8.01-7.85 (m, 2H), 7.34 (d, J=12.3 Hz, 1H), 6.68 (d, J=7.8 Hz, 1H), 4.33 (s, 3H), 3.28-3.19 (m, 4H), 2.65 (d, J=12.7 Hz, 2H), 2.35 (s, 3H).

### Example 278: Synthesis of Compound 565

### Synthesis of Intermediate C450

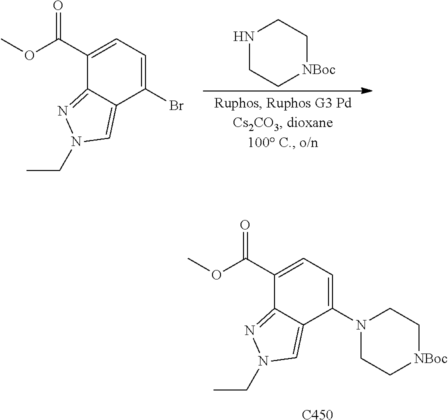 [SMILES: CCn1cc2c(Br)ccc(C(=O)OC)c2n1 ; CC(C)(C)OC(=O)N1CCNCC1 ; CCn1cc2c(N3CCN(C(=O)OC(C)(C)C)CC3)ccc(C(=O)OC)c2n1 ; "C450"]

To a stirred mixture of methyl 4-bromo-2-ethylindazole-7-carboxylate (1 g, 3.532 mmol, 1 equiv), tert-butyl piperazine-1-carboxylate (0.79 g, 4.238 mmol, 1.2 equiv) and Cs₂CO₃ (3.45 g, 10.596 mmol, 3 equiv) in 1,4-dioxane (20 mL) were added RuPhos (0.33 g, 0.706 mmol, 0.2 equiv) and RuPhos Palladacycle Gen.3 (0.30 g, 0.353 mmol, 0.1 equiv) at room temperature under nitrogen atmosphere. The resulting mixture was stirred overnight at 100° C. under nitrogen atmosphere. The resulting mixture was concentrated under vacuum. The residue was purified by silica gel column chromatography, eluted with PE/EA (1:1) to afford methyl 4-[4-(tert-butoxycarbonyl)piperazin-1-yl]-2-ethylindazole-7-carboxylate (1.1 g, 80%) as a solid. LCMS (ES, m/z): 389[M+H]⁺

### Synthesis of Intermediate C451

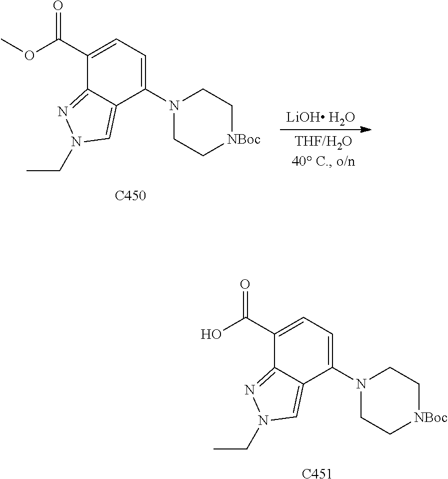

To a stirred solution of methyl 4-[4-(tert-butoxycarbonyl)piperazin-1-yl]-2-ethylindazole-7-carboxylate (1.1 g, 2.832 mmol, 1 equiv) in THF/H₂O (9 mL/3 mL) was added lithiumol hydrate (0.36 g, 8.496 mmol, 3 equiv) at room temperature. The resulting mixture was stirred overnight at 40° C. The resulting mixture was concentrated under vacuum. The resulting mixture was diluted with H₂O (10 mL). The mixture was acidified to pH 4 with citric acid. The precipitated solids were collected by filtration and dried under infrared light to afford 4-[4-(tert-butoxycarbonyl)piperazin-1-yl]-2-ethylindazole-7-carboxylic acid (850 mg, 80%) as a solid. LCMS (ES, m/z): 375 [M+H]

### Synthesis of Intermediate C452

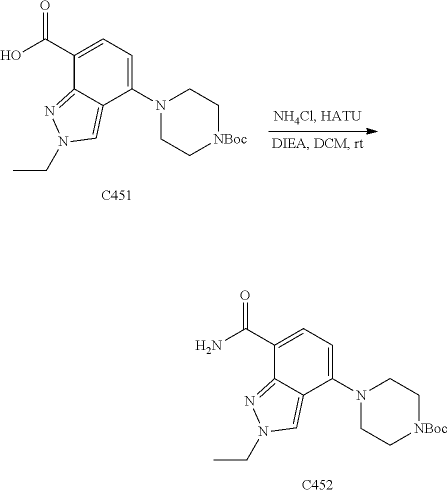 [SMILES: CCn1cc2c(N3CCN(C(=O)OC(C)(C)C)CC3)ccc(C(=O)O)c2n1 ; "C451" ; CCn1cc2c(N3CCN(C(=O)OC(C)(C)C)CC3)ccc(C(N)=O)c2n1 ; "C452"]

To a stirred solution of 4-[4-(tert-butoxycarbonyl)piperazin-1-yl]-2-ethylindazole-7-carboxylic acid (850 mg, 2.270 mmol, 1 equiv) and HATU (1035.78 mg, 2.724 mmol, 1.2 equiv) in DCM (20 mL) was added DIEA (1466.99 mg, 11.350 mmol, 5 equiv) and NH₄Cl (485.70 mg, 9.080 mmol, 4 equiv) at room temperature. The resulting mixture was stirred overnight at room temperature. The resulting mixture was diluted with DCM (100 mL), washed with 2×50 mL of H₂O and 1×50 mL of brine. The organic layer was dried over anhydrous Na₂SO₄. After filtration, the filtrate was concentrated under reduced pressure. The residue was purified by silica gel column chromatography, eluted with CH₂Cl₂/MeOH (10:1) to afford tert-butyl 4-(7-carbamoyl-2-ethylindazol-4-yl)piperazine-1-carboxylate (700 mg, 83%) as a solid. LCMS (ES, m/z): 374 [M+H]⁺

### Synthesis of Intermediate C453

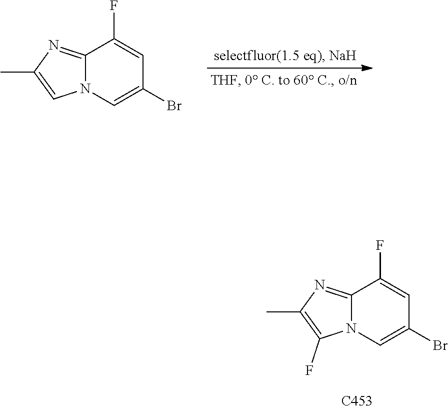

To a stirred solution of 6-bromo-8-fluoro-2-methylimidazo[1,2-a]pyridine (1.0 g, 4.366 mmol, 1 equiv) in THF (20 mL) was added NaH (0.16 g, 6.549 mmol, 1.5 equiv) at 0° C. The resulting mixture was stirred for 0.5 h at 0° C. under nitrogen atmosphere. To the above mixture was added F-TEDA-BF₄ (2.32 g, 6.549 mmol, 1.5 equiv) in portions at 0° C. The resulting mixture was stirred for additional 16 h at 60° C. The reaction was quenched with MeOH at room temperature. The resulting mixture was concentrated under vacuum. The residue was purified by silica gel column chromatography, eluted with CH₂Cl₂/MeOH (10:1) to afford 6-bromo-3,8-difluoro-2-methylimidazo[1,2-a]pyridine (400 mg, 37.09%) as a solid. LCMS (ES, m/z): 247 [M+H]⁺

### Synthesis of Intermediate C454

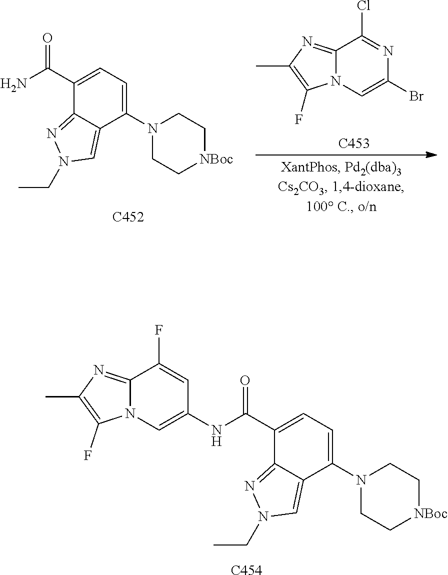

To a stirred mixture of tert-butyl 4-(7-carbamoyl-2-ethylindazol-4-yl)piperazine-1-carboxylate (160 mg, 0.428 mmol, 1 equiv), Cs₂CO₃ (418.77 mg, 1.284 mmol, 3 equiv) and 6-bromo-3,8-difluoro-2-methylimidazo[1,2-a]pyridine (127.01 mg, 0.514 mmol, 1.2 equiv) in 1,4-dioxane (3 mL) were added XantPhos (49.58 mg, 0.086 mmol, 0.2 equiv) and Pd₂(dba)₃ (39.23 mg, 0.043 mmol, 0.1 equiv) at room temperature under nitrogen atmosphere. The resulting mixture was stirred overnight at 100° C. under nitrogen atmosphere. The resulting mixture was concentrated under vacuum. The residue was purified by silica gel column chromatography, eluted with CH₂Cl₂/MeOH (10:1) to afford tert-butyl 4-[7-({3,8-difluoro-2-methylimidazo[1,2-a]pyridin-6-yl}carbamoyl)-2-ethylindazol-4-yl]piperazine-1-carboxylate (100 mg, 43%) as a solid. LCMS (ES, m/z): 540 [M+H]⁺

### Synthesis of Compound 565

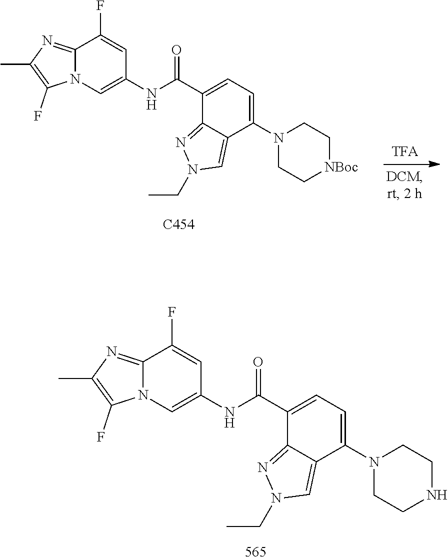

To a stirred solution of tert-butyl 4-[7-({3,8-difluoro-2-methylimidazo[1,2-a]pyridin-6-yl}carbamoyl)-2-ethylindazol-4-yl]piperazine-1-carboxylate (100 mg, 0.185 mmol, 1 equiv) in DCM (1 mL) was added TFA (1 mL) at room temperature. The resulting mixture was stirred for 2 h at room temperature. The resulting mixture was concentrated under vacuum. The crude product was purified by Prep-HPLC (Condition 15, Gradient 1) to afford N-{3,8-difluoro-2-methylimidazo[1,2-a]pyridin-6-yl}-2-ethyl-4-(piperazin-1-yl) indazole-7-carboxamide; trifluoroacetic acid (19 mg, 19%) as solid. LCMS (ES, m/z): 440 [M+H]^{+ 1}H NMR (400 MHz, DMSO-d₆) δ 11.21 (s, 1H), 8.99-8.90 (m, 2H), 8.82 (s, 2H), 8.03 (d, J=8.0 Hz, 1H), 7.37 (d, J=12.6 Hz, 1H), 6.62 (d, J=8.1 Hz, 1H), 4.63 (q, J=7.2 Hz, 2H), 3.60 (t, J=5.1 Hz, 4H), 3.36 (s, 4H), 2.35 (d, J=1.3 Hz, 3H), 1.64 (t, J=7.3 Hz, 3H).

### Example 279: Synthesis of Compound 566

### Synthesis of Intermediate C455

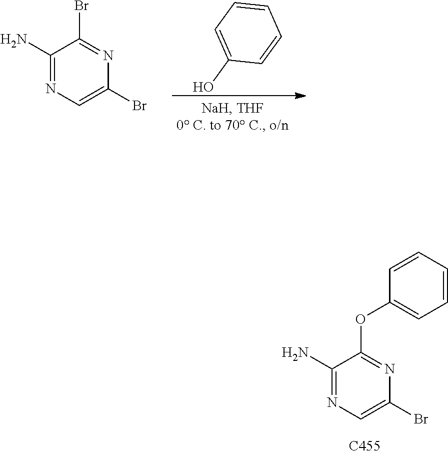

To a solution of phenol (4.30 g, 45.671 mmol, 1.1 equiv) in THF (55 mL) was added sodium hydride (60% in oil, 1.83 g, 1.1 equiv) at 0° C. The mixture was stirred for 30 min. The solution of 3,5-dibromopyrazin-2-amine (10.5 g, 41.519 mmol, 1 equiv) in THF (50 mL) was added and the mixture was allowed to warm to 70° C. and stirred overnight. The mixture was allowed to cool down to room temperature. The reaction was quenched with water/ice. The resulting mixture was extracted with EtOAc (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (1:1) to afford 5-bromo-3-phenoxypyrazin-2-amine (2.3 g, 21%) as a solid. LCMS (ES, m/z): 266 [M+H]⁺

### Synthesis of Intermediate C456

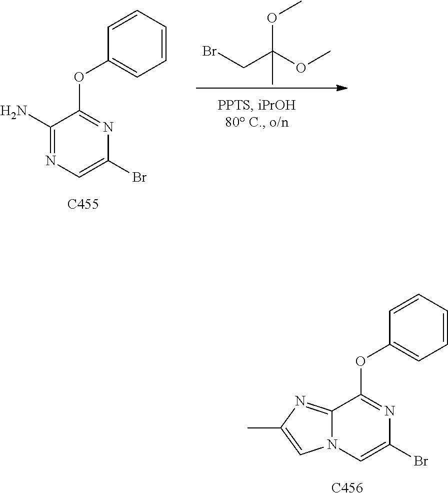

To a stirred solution of 5-bromo-3-phenoxypyrazin-2-amine (2.3 g, 8.643 mmol, 1 equiv) in isopropanol (23 mL) were added 1-bromo-2,2-dimethoxypropane (1.90 g, 10.372 mmol, 1.2 equiv) and PPTS (0.22 g, 0.864 mmol, 0.1 equiv) at room temperature. The resulting mixture was stirred overnight at 80° C. The mixture was allowed to cool down to room temperature. The precipitated solids were collected by filtration and the solids was dried under vacuum to afford 6-bromo-2-methyl-8-phenoxyimidazo[1,2-a]pyrazine (800 mg, 30%) as a solid. LCMS (ES, m/z): 304 [M+H]⁺

### Synthesis of Intermediate C457

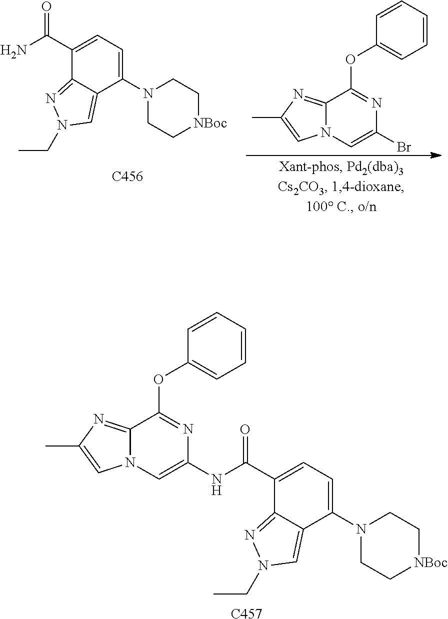

To a stirred mixture of tert-butyl 4-(7-carbamoyl-2-ethylindazol-4-yl)piperazine-1-carboxylate (160 mg, 0.428 mmol, 1 equiv), 6-bromo-2-methyl-8-phenoxyimidazo[1,2-a]pyrazine (156.37 mg, 0.514 mmol, 1.2 equiv) and Cs₂CO₃ (418.77 mg, 1.284 mmol, 3 equiv) in 1,4-dioxane (3.2 mL) was added XantPhos (49.58 mg, 0.086 mmol, 0.2 equiv) and Pd₂(dba)₃ (39.23 mg, 0.043 mmol, 0.1 equiv) at room temperature under nitrogen atmosphere. The resulting mixture was stirred overnight at 100° C. under nitrogen atmosphere. The resulting mixture was concentrated under vacuum. The residue was purified by silica gel column chromatography, eluted with CH₂Cl₂/MeOH (10:1) to afford tert-butyl 4-[2-ethyl-7-({2-methyl-8-phenoxyimidazo[1,2-a]pyrazin-6-yl}carbamoyl) indazol-4-yl]piperazine-1-carboxylate (110 mg, 43%) as a solid. LCMS (ES, m/z): 597 [M+H]⁺

### Synthesis of Compound 566

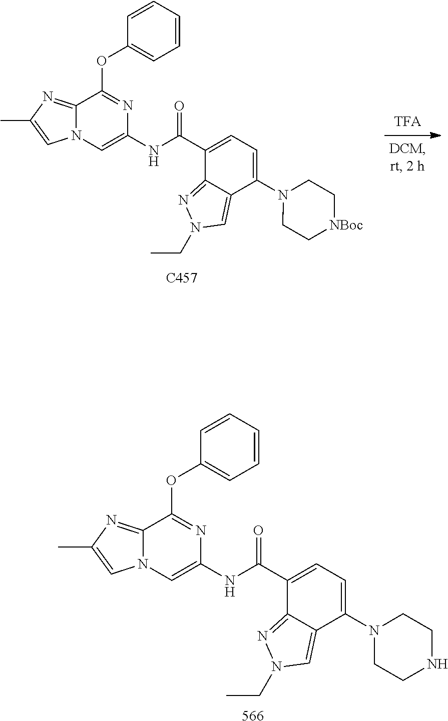

To a stirred solution of tert-butyl 4-[2-ethyl-7-({2-methyl-8-phenoxyimidazo[1,2-a]pyrazin-6-yl}carbamoyl) indazol-4-yl]piperazine-1-carboxylate (110 mg, 0.184 mmol, 1 equiv) in DCM (1 mL) was added TFA (1 mL) at room temperature. The resulting mixture was stirred for 2 h at room temperature. The resulting mixture was concentrated under vacuum. The crude product was purified by Prep-HPLC (Condition 10, Gradient 15) to afford 2-ethyl-N-{2-methyl-8-phenoxyimidazo[1,2-a]pyrazin-6-yl}-4-(piperazin-1-yl) indazole-7-carboxamide (17.1 mg, 19%) as a solid. LCMS (ES, m/z): 497 [M+H]^{+ 1}H NMR (400 MHz, DMSO-d₆) δ 11.23 (s, 1H), 9.09 (s, 1H), 8.75 (s, 1H), 8.04 (s, 1H), 7.96 (d, J=8.1 Hz, 1H), 7.53 (t, J=7.8 Hz, 2H), 7.37 (dd, J=13.8, 7.4 Hz, 3H), 6.46 (d, J=8.1 Hz, 1H), 4.30 (q, J=7.3 Hz, 2H), 3.34 (d, J=4.9 Hz, 4H) 2.90 (dd, J=6.1, 3.6 Hz, 4H), 2.41 (s, 4H), 1.34 (t, J=7.3 Hz, 3H), 1.24 (s, 1H).

### Example 280: Synthesis of Compound 568

### Synthesis of Intermediate C458

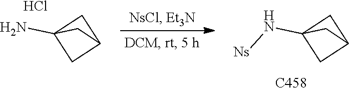

To a stirred mixture of bicyclo[1.1.1]pentan-1-amine hydrochloride (550 mg, 4.599 mmol, 1 equiv) in DCM (10 mL) were added Et₃N (1.40 g, 13.797 mmol, 3 equiv) and 4-nitrobenzene-1-sulfonyl chloride (1.12 g, 5.059 mmol, 1.1 equiv) in portions at room temperature under nitrogen atmosphere. The resulting mixture was stirred for 5 h at room temperature under nitrogen atmosphere. The resulting mixture was washed with 3×10 mL of water. dried over anhydrous Na₂SO₄. After filtration, the filtrate was concentrated under reduced pressure to afford N-{bicyclo[1.1.1]pentan-1-yl}-4-nitrobenzenesulfonamide (780 mg, 63%) as a solid. LCMS (ES, m/z): 267 [M−H]⁻

### Synthesis of Intermediate C459

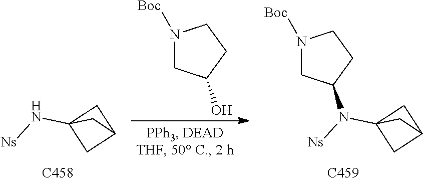

To a stirred mixture of N-{bicyclo[1.1.1]pentan-1-yl}-4-nitrobenzenesulfonamide (750 mg, 2.795 mmol, 1.0 equiv) and tert-butyl (3S)-3-hydroxypyrrolidine-1-carboxylate (785.1 mg, 4.192 mmol, 1.5 equiv) in THF (15 mL) were added PPh₃ (1.4 g, 5.590 mmol, 2.0 equiv) and DEAD (973.7 mg, 5.590 mmol, 2.0 equiv) dropwise at 0° C. under nitrogen atmosphere. The resulting mixture was stirred for 2 h at 50° C. under nitrogen atmosphere. The resulting mixture was diluted with water (10 mL). The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (1:1) to afford tert-butyl (3R)-3-(N-{bicyclo[1.1.1]pentan-1-yl}4-nitrobenzenesulfonamido)pyrrolidine-1-carboxylate (760 mg, 62%) as a solid. LCMS (ES, m/z): 438 [M+H]⁺

### Synthesis of Intermediate C460

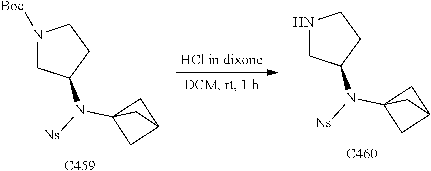

To a stirred mixture of tert-butyl (3R)-3-(N-{bicyclo[1.1.1]pentan-1-yl}4-nitrobenzenesulfonamido)pyrrolidine-1-carboxylate (320 mg, 0.731 mmol, 1 equiv) in DCM (2 mL) was added HCl(gas) in 1,4-dioxane (0.5 mL, 4M) dropwise at room temperature. The resulting mixture was stirred for 1 h at room temperature. The resulting mixture was concentrated under vacuum. The mixture basified to pH 8 with saturated NaHCO₃ (aq.). The resulting mixture was extracted with EtOAc (3×2 mL). The combined organic layers were washed with water (3×2 mL), dried over anhydrous Na₂SO₄. After filtration, the filtrate was concentrated under reduced pressure to afford N-{bicyclo[1.1.1]pentan-1-yl}-4-nitro-N-[(3R)-pyrrolidin-3-yl]benzenesulfonamide (230 mg, 93%) as a solid. LCMS (ES, m/z): 338 [M+H]⁺

### Synthesis of Intermediate C461

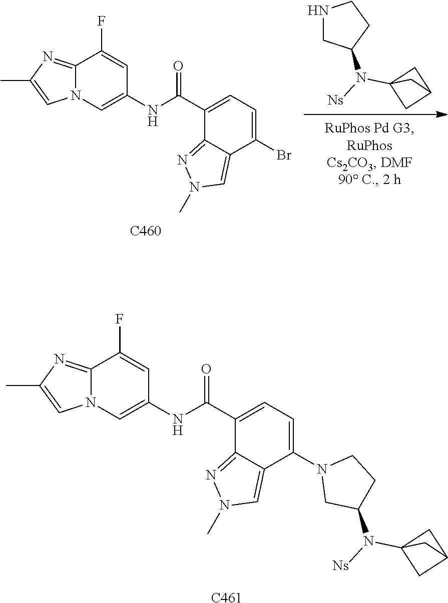

To a stirred mixture of 4-bromo-N-{8-fluoro-2-methylimidazo[1,2-a]pyridin-6-yl}-2-methylindazole-7-carboxamide (110 mg, 0.273 mmol, 1.0 equiv) and N-{bicyclo[1.1.1]pentan-1-yl}-4-nitro-N-[(3R)-pyrrolidin-3-yl]benzenesulfonamide (119.95 mg, 0.355 mmol, 1.3 equiv) in DMF (2 mL) were added Cs₂CO₃ (222.7 mg, 0.683 mmol, 2.5 equiv) and RuPhos (25.5 mg, 0.055 mmol, 0.2 equiv) and RuPhos Palladacycle Gen.3 (22.9 mg, 0.027 mmol, 0.1 equiv) at room temperature under nitrogen atmosphere. The resulting mixture was stirred for 2 h at 90° C. under nitrogen atmosphere. The mixture was allowed to cool down to room temperature. The resulting mixture was diluted with water (5 mL). The resulting mixture was extracted with EtOAc (3×5 mL). The combined organic layers were washed with water (3×5 mL), dried over anhydrous Na₂SO₄. After filtration, the filtrate was concentrated under reduced pressure. The residue was purified by silica gel column chromatography, eluted with EA to afford 4-[(3R)-3-(N-{bicyclo[1.1.1]pentan-1-yl}4-nitrobenzenesulfonamido)pyrrolidin-1-yl]-N-{8-fluoro-2-methylimidazo[1,2-a]pyridin-6-yl}-2-methylindazole-7-carboxamide (60 mg, 33%) as a solid. LCMS (ES, m/z): 658 [M+H]⁺

### Synthesis of Compound 568

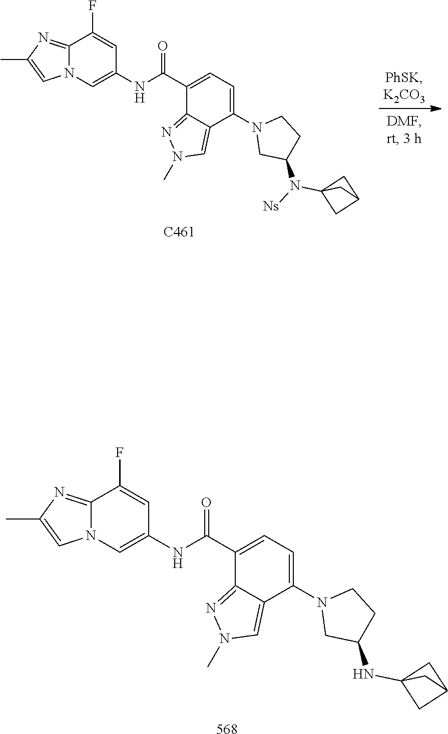

To a stirred mixture of 4-[(3R)-3-(N-{bicyclo[1.1.1]pentan-1-yl}4-nitrobenzenesulfonamido)pyrrolidin-1-yl]-N-{8-fluoro-2-methylimidazo[1,2-a]pyridin-6-yl}-2-methylindazole-7-carboxamide (40 mg, 0.061 mmol, 1 equiv) in DMF (1 mL) were added K₂CO₃ (16.8 mg, 0.122 mmol, 2 equiv) and PhSK (13.4 mg, 0.092 mmol, 1.5 equiv) at room temperature. The resulting mixture was stirred for 3 h at room temperature. The resulting mixture was diluted with water (5 mL). The resulting mixture was extracted with EtOAc (3×5 mL). The combined organic layers were washed with water (3×5 mL), dried over anhydrous Na₂SO₄. After filtration, the filtrate was concentrated under reduced pressure. The crude product was purified by Prep-HPLC (Condition 10, Gradient 2) to afford 4-[(3R)-3-{bicyclo[1.1.1]pentan-1-ylamino}pyrrolidin-1-yl]-N-{8-fluoro-2-methylimidazo[1,2-a]pyridin-6-yl}-2-methylindazole-7-carboxamide (11 mg, 38%) as a solid. LCMS (ES, m/z): 558 [M+H]^{+ 1}H NMR (300 MHz, DMSO-d₆) δ 11.01 (s, 1H), 9.19 (s, 1H), 8.79 (s, 1H), 7.91 (dd, J=12.8, 5.7 Hz, 2H), 7.30 (d, J=12.2 Hz, 1H), 6.00 (d, J=8.4 Hz, 1H), 4.27 (s, 3H), 3.93-3.44 (m, 4H), 2.36 (d, J=7.6 Hz, 5H), 2.28-2.27 (m, 1H), 1.79-1.78 (m, 7H).

### Example 281: Synthesis of Compound 569

### Synthesis of Intermediate C462

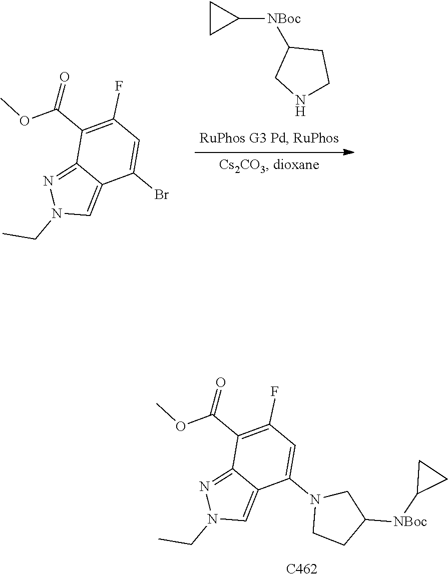

To a stirred solution of methyl 4-bromo-2-ethyl-6-fluoroindazole-7-carboxylate (500 mg, 1.66 mmol, 1 equiv), Cs₂CO₃ (1082 mg, 3.32 mmol, 2 equiv) and tert-butyl N-cyclopropyl-N-(pyrrolidin-3-yl)carbamate (752 mg, 3.32 mmol, 2 equiv) in dioxane (10 mL) were added RuPhos (775 mg, 1.66 mmol, 1 equiv) and RuPhos Palladacycle Gen.3 (278 mg, 0.332 mmol, 0.2 equiv) at room temperature under nitrogen atmosphere. The resulting mixture was stirred for overnight at 100° C. under nitrogen atmosphere. The mixture was allowed to cool down to room temperature. The reaction was quenched by the addition of water (30 mL) at room temperature. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by reverse flash chromatography (Condition 3, Gradient 1) to afford methyl 4-{3-[(tert-butoxycarbonyl)(cyclopropyl)amino]pyrrolidin-1-yl}-2-ethyl-6-fluoroindazole-7-carboxylate (150 mg, 20%) as a solid. LCMS (ES, m/z): 447 [M+H]⁺

### Synthesis of Intermediate C463

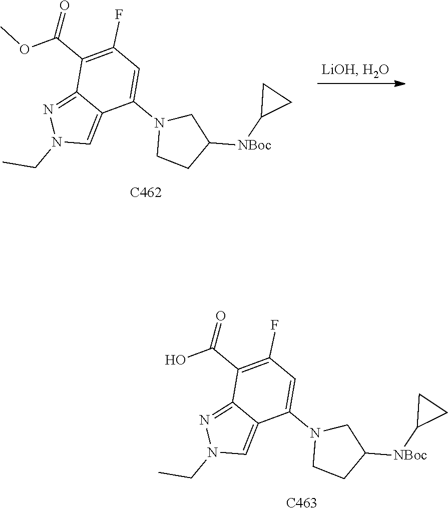

A mixture of methyl 4-{3-[(tert-butoxycarbonyl)(cyclopropyl)amino]pyrrolidin-1-yl}-2-ethyl-6-fluoroindazole-7-carboxylate (150 mg, 0.336 mmol, 1 equiv) and LiOH·H₂O (282 mg, 6.720 mmol, 20 equiv) in H₂O (2 mL) and THF (4 mL) was stirred for 24 h at room temperature. The resulting mixture was concentrated under vacuum. The residue was diluted with water (5.0 mL), then adjusted to pH 5 with 1 mol/L aq. HCl. The resulting mixture was extracted with CH₂Cl₂ (3×5 mL). The combined organic layers were washed with brine (1×5 mL), dried over anhydrous Na₂SO₄. After filtration, the filtrate was concentrated under reduced pressure to afford 4-{3-[(tert-butoxycarbonyl)(cyclopropyl)amino]pyrrolidin-1-yl}-2-ethyl-6-fluoroindazole-7-carboxylic acid (130 mg, 89%) as an oil. LCMS (ES, m/z): 433 [M+H]⁺

### Synthesis of Intermediate C464

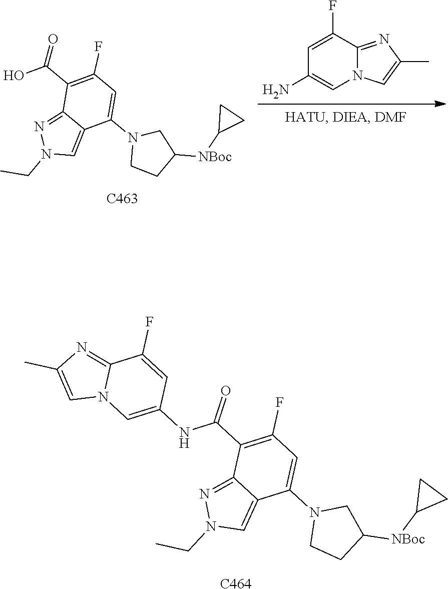

To a stirred solution of 4-{3-[(tert-butoxycarbonyl)(cyclopropyl)amino]pyrrolidin-1-yl}-2-ethyl-6-fluoroindazole-7-carboxylic acid (130 mg, 0.301 mmol, 1 equiv) and 8-fluoro-2-methylimidazo[1,2-a]pyridin-6-amine (55 mg, 0.331 mmol, 1.1 equiv) in DMF (5 mL) were added HATU (137 mg, 0.361 mmol, 1.2 equiv) and DIEA (117 mg, 0.903 mmol, 3 equiv) at room temperature. The reaction was quenched by the addition of water (20 mL) at room temperature. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:3) to afford tert-butyl N-cyclopropyl-N-{1-[2-ethyl-6-fluoro-7-({8-fluoro-2-methylimidazo[1,2-a]pyridin-6-yl}carbamoyl) indazol-4-yl]pyrrolidin-3-yl}carbamate (130 mg, 75%) as a solid. LCMS (ES, m/z): 580 [M+H]⁺

### Synthesis of Compound 569

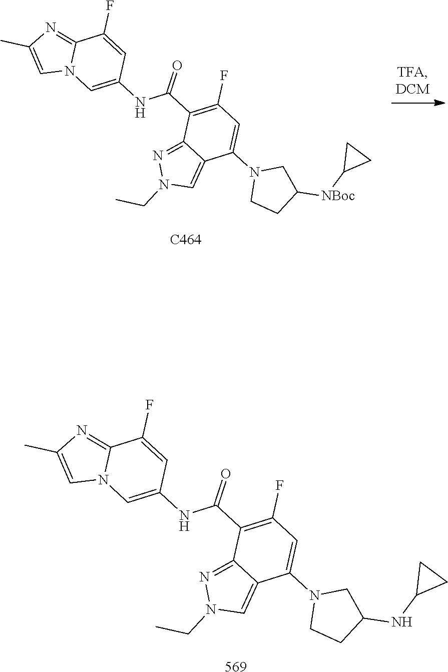 [SMILES: CCn1cc2c(N3CCC(N(C(=O)OC(C)(C)C)C4CC4)C3)cc(F)c(C(=O)Nc3cc(F)c4nc(C)cn4c3)c2n1 ; "C464" ; CCn1cc2c(N3CCC(NC4CC4)C3)cc(F)c(C(=O)Nc3cc(F)c4nc(C)cn4c3)c2n1 ; "569"]

A solution of tert-butyl N-cyclopropyl-N-{1-[2-ethyl-6-fluoro-7-({8-fluoro-2-methylimidazo[1,2-a]pyridin-6-yl}carbamoyl) indazol-4-yl]pyrrolidin-3-yl}carbamate (130 mg, 0.224 mmol, 1 equiv) in TFA (1 mL) and DCM (3 mL) was stirred for 2 h at room temperature. The resulting mixture was concentrated under reduced pressure. The residue was basified to pH 8 with 7M NH(g) in MeOH. The resulting mixture was concentrated under vacuum. The residue was purified by reverse flash chromatography (Condition 3, Gradient 3) to afford 4-[3-(cyclopropylamino)pyrrolidin-1-yl]-2-ethyl-6-fluoro-N-{8-fluoro-2-methylimidazo[1,2-a]pyridin-6-yl}indazole-7-carboxamide (27 mg, 25%) as a solid. LCMS (ES, m/z): 480 [M+H]^{+ 1}H NMR (300 MHz, DMSO-d₆) δ 11.07 (s, 1H), 9.17 (d, J=1.7 Hz, 1H), 8.80 (s, 1H), 7.87 (d, J=3.1 Hz, 1H), 7.18 (dd, J=12.4, 1.7 Hz, 1H), 5.74 (d, J=16.1 Hz, 1H), 4.52 (q, J=7.3 Hz, 2H), 3.85-3.48 (m, 4H), 3.44 (d, J=10.1 Hz, 1H), 2.35 (s, 3H), 2.15 (tt, J=7.0, 3.5 Hz, 2H), 1.98 (p, J=6.1 Hz, 1H), 1.58 (t, J=7.2 Hz, 3H), 0.42 (d, J=6.6 Hz, 2H), 0.26 (dq, J=7.2, 4.8, 3.3 Hz, 2H).

### Example 282: Synthesis of Compound 571

### Synthesis of Intermediate C465

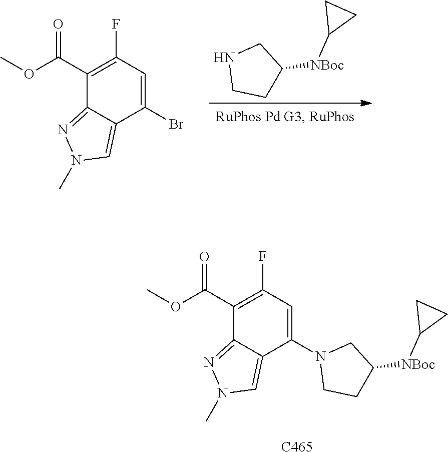

To a solution of methyl 4-bromo-6-fluoro-2-methylindazole-7-carboxylate (200 mg, 0.697 mmol, 1 equiv) and tert-butyl N-cyclopropyl-N-[(3R)-pyrrolidin-3-yl]carbamate (173 mg, 0.767 mmol, 1.1 equiv) in dioxane (6 mL) were added Cs₂CO₃ (680 mg, 2.091 mmol, 3.0 equiv), RuPhos (32.5 mg, 0.07 mmol, 0.1 equiv) and RuPhos Palladacycle Gen.3 (58.2 mg, 0.070 mmol, 0.1 equiv). After stirring for 2 h at 80° C. under a nitrogen atmosphere, the resulting mixture was concentrated under reduced pressure. The residue was purified by reverse flash chromatography (Condition 5, Gradient 2) to afford methyl 4-[(3R)-3-[(tert-butoxycarbonyl)(cyclopropyl)amino]pyrrolidin-1-yl]-6-fluoro-2-methylindazole-7-carboxylate (210 mg, 70%) as a solid. LCMS (ES, m/z): 433 [M+H]⁺

### Synthesis of Intermediate C466

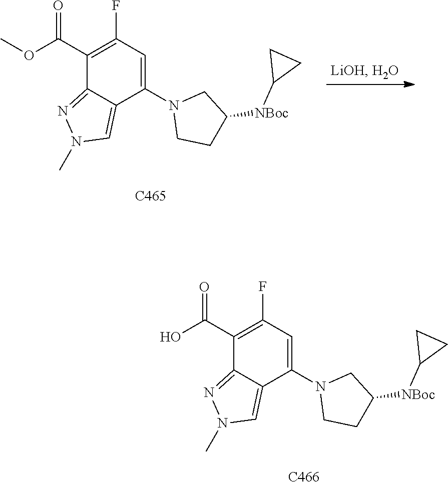 [SMILES: COC(=O)c1c(F)cc(N2CC[C@@H](N(C(=O)OC(C)(C)C)C3CC3)C2)c2cn(C)nc12 ; "C465" ; Cn1cc2c(N3CC[C@@H](N(C(=O)OC(C)(C)C)C4CC4)C3)cc(F)c(C(=O)O)c2n1 ; "C466"]

A mixture of methyl 4-[(3R)-3-[(tert-butoxycarbonyl)(cyclopropyl)amino]pyrrolidin-1-yl]-6-fluoro-2-methylindazole-7-carboxylate (200 mg, 0.462 mmol, 1 equiv) and LiOH·H₂O (66 mg, 2.772 mmol, 6.0 equiv) in H₂O (2 mL), THF (2 mL) and MeOH (2 mL) was stirred for 12 h at 60° C. The mixture was acidified to pH 3 with aq. HCl (2 M). The resulting mixture was concentrated under reduced pressure. The residue was purified by reverse flash chromatography (Condition 5, Gradient 2) to afford 4-[(3R)-3-[(tert-butoxycarbonyl)(cyclopropyl)amino]pyrrolidin-1-yl]-6-fluoro-2-methylindazole-7-carboxylic acid (100 mg, 52%) as a solid. LCMS (ES, m/z): 419 [M+H]⁺

### Synthesis of Intermediate C467

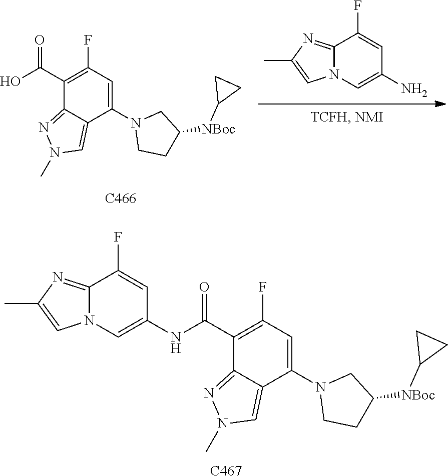

To a stirred solution of 4-[(3R)-3-[(tert-butoxycarbonyl)(cyclopropyl)amino]pyrrolidin-1-yl]-6-fluoro-2-methylindazole-7-carboxylic acid (100 mg, 0.239 mmol, 1 equiv) and TCFH (109 mg, 0.287 mmol, 1.2 equiv) in CH₃CN (2 mL) were added NMI (123.5 mg, 0.956 mmol, 4.0 equiv) and 8-fluoro-2-methylimidazo[1,2-a]pyridin-6-amine (47.3 mg, 0.287 mmol, 1.2 equiv) in portions at room temperature. The resulting mixture was stirred for 12 h at room temperature. The resulting mixture was concentrated under reduced pressure. The residue was purified by reverse flash chromatography (Condition 5, Gradient 3) tert-butyl N-cyclopropyl-N-[(3R)-1-[6-fluoro-7-({8-fluoro-2-methylimidazo[1,2-a]pyridin-6-yl}carbamoyl)-2-methylindazol-4-yl]pyrrolidin-3-yl]carbamate (20 mg, 15%) as a solid. LCMS (ES, m/z): 566 [M+H]⁺

### Synthesis of Compound 571

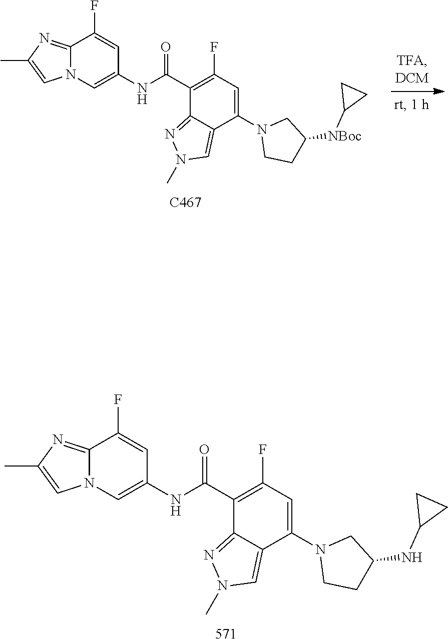

To a stirred solution of tert-butyl N-cyclopropyl-N-[(3R)-1-[6-fluoro-7-({8-fluoro-2-methylimidazo[1,2-a]pyridin-6-yl}carbamoyl)-2-methylindazol-4-yl]pyrrolidin-3-yl]carbamate (15 mg, 0.027 mmol, 1 equiv) in DCM (2 mL) was added TFA (0.05 mL) at room temperature. The resulting mixture was stirred for 1 h at room temperature. The mixture was basified to pH 8 with NH₃(g) in MeOH. The resulting mixture was concentrated under reduced pressure. The residue was purified by reverse flash chromatography (Condition 3, Gradient 5) to afford 4-[(3R)-3-(cyclopropylamino)pyrrolidin-1-yl]-6-fluoro-N-{8-fluoro-2-methylimidazo[1,2-a]pyridin-6-yl}-2-methylindazole-7-carboxamide (4 mg, 32%) as a solid. LCMS (ES, m/z): 466 [M+H]^{+ 1}H NMR (300 MHz, DMSO-d₆) δ 11.03 (s, 1H), 9.18 (d, J=1.6 Hz, 1H), 8.80 (s, 1H), 7.91-7.83 (m, 1H), 7.25 (dd, J=12.5, 1.7 Hz, 1H), 5.76 (d, J=16.1 Hz, 1H), 4.22 (s, 3H), 3.72 (d, J=14.9 Hz, 3H), 3.65-3.51 (m, 2H), 2.38-2.31 (m, 3H), 2.16 (dd, J=8.3, 4.5 Hz, 2H), 1.98 (dd, J=12.2, 6.1 Hz, 1H), 0.42 (dd, J=6.6, 1.5 Hz, 2H), 0.25 (s, 2H).

### Example 283: Synthesis of Compound 573

### Synthesis of Intermediate C468

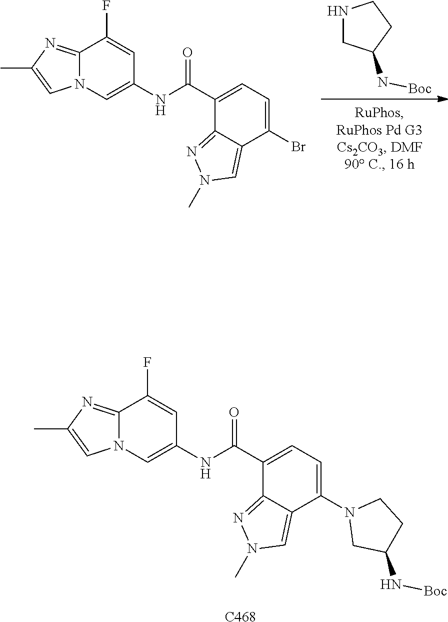

To a stirred mixture of 4-bromo-N-{8-fluoro-2-methylimidazo[1,2-a]pyridin-6-yl}-2-methylindazole-7-carboxamide (220 mg, 0.547 mmol, 1 equiv) and tert-butyl N-[(3R)-pyrrolidin-3-yl]carbamate (122.3 mg, 0.656 mmol, 1.2 equiv) in DMF (5.5 mL) were added Cs₂CO₃ (534.6 mg, 1.641 mmol, 3 equiv) and Ruphos (51.1 mg, 0.109 mmol, 0.2 equiv) and RuPhos Palladacycle Gen.3 (45.8 mg, 0.055 mmol, 0.1 equiv) at room temperature under nitrogen atmosphere. The resulting mixture was stirred for 16 h at 90° C. under nitrogen atmosphere. The mixture was allowed to cool down to room temperature. The resulting mixture was diluted with water (15 mL). The resulting mixture was extracted with EtOAc (3×15 mL). The combined organic layers were washed with water (3×10 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-[(3R)-1-[7-({8-fluoro-2-methylimidazo[1,2-a]pyridin-6-yl}carbamoyl)-2-methylindazol-4-yl]pyrrolidin-3-yl]carbamate (200 mg, 72%) as a solid. LCMS (ES, m/z): 508 [M+H]⁺

### Synthesis of Intermediate C469

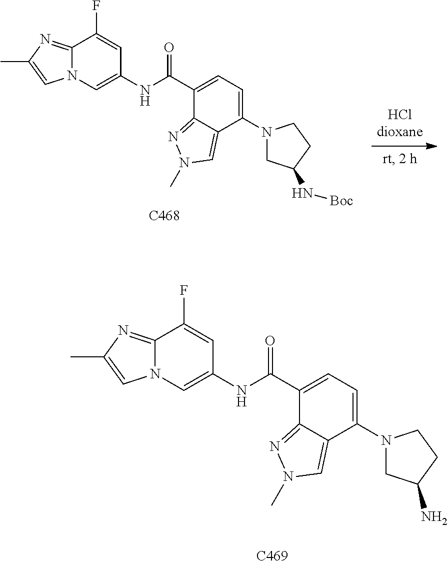

To a stirred mixture of tert-butyl N-[(3R)-1-[7-({8-fluoro-2-methylimidazo[1,2-a]pyridin-6-yl}carbamoyl)-2-methylindazol-4-yl]pyrrolidin-3-yl]carbamate (170 mg, 0.335 mmol, 1 equiv) in DCM (2.5 mL) was added HCl(gas) in 1,4-dioxane (1 mL) dropwise at room temperature. The resulting mixture was stirred for 2 h at room temperature. The resulting mixture was concentrated under reduced pressure to afford 4-[(3R)-3-aminopyrrolidin-1-yl]-N-{8-fluoro-2-methylimidazo[1,2-a]pyridin-6-yl}-2-methylindazole-7-carboxamide hydrochloride (140 mg, 103%) as a solid. LCMS (ES, m/z): 408 [M+H]⁺

### Synthesis of Compound 573

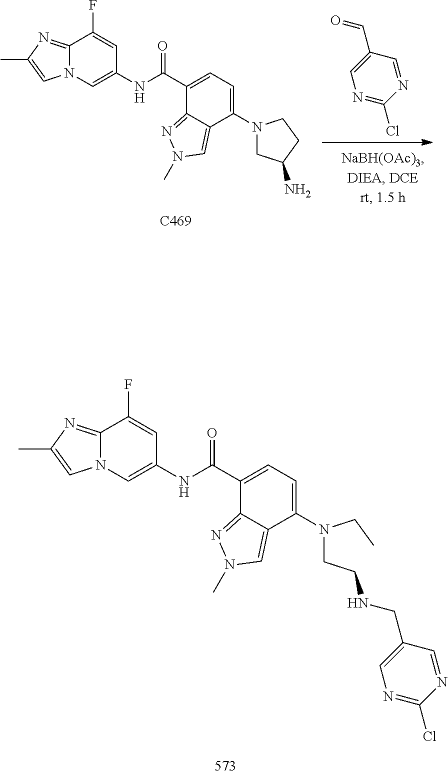

To a stirred mixture of 4-[(3R)-3-aminopyrrolidin-1-yl]-N-{8-fluoro-2-methylimidazo[1,2-a]pyridin-6-yl}-2-methylindazole-7-carboxamide (76.2 mg, 0.172 mmol, 1.0 equiv) and DIEA (24.4 mg, 0.189 mmol, 1.1 equiv) in DCE (1.75 mL) were added 2-chloropyrimidine-5-carbaldehyde (24.49 mg, 0.172 mmol, 1.0 equiv) and NaBH(OAc)₃ (54.6 mg, 0.258 mmol, 1.5 equiv) at room temperature. The resulting mixture was stirred for 1.5 h at room temperature. The reaction was quenched with water at 0° C. The resulting mixture was concentrated under vacuum. The crude product was purified by Prep-HPLC (Condition 10, Gradient 2) to afford 4-[(3R)-3-{[(2-chloropyrimidin-5-yl)methyl]amino}pyrrolidin-1-yl]-N-{8-fluoro-2-methylimidazo[1,2-a]pyridin-6-yl}-2-methylindazole-7-carboxamide (13.5 mg, 15%) as a solid. LCMS (ES, m/z): 534 [M+H]^{+ 1}H NMR (300 MHz, DMSO-d₆) δ 11.01 (s, 1H), 9.20 (s, 1H), 8.80 (d, J=16.3 Hz, 3H), 7.97-7.80 (m, 2H), 7.31 (d, J=12.3 Hz, 1H), 6.02 (d, J=8.4 Hz, 1H), 4.28 (s, 3H), 3.92-3.59 (m, 5H), 3.47 (s, 2H), 2.75-2.72 (m, 1H), 2.35 (s, 3H), 2.23 (d, J=31.4 Hz, 1H), 1.98-1.97 (m, 1H).

### Example 284: Synthesis of Compound 575

### Synthesis of Intermediate C470

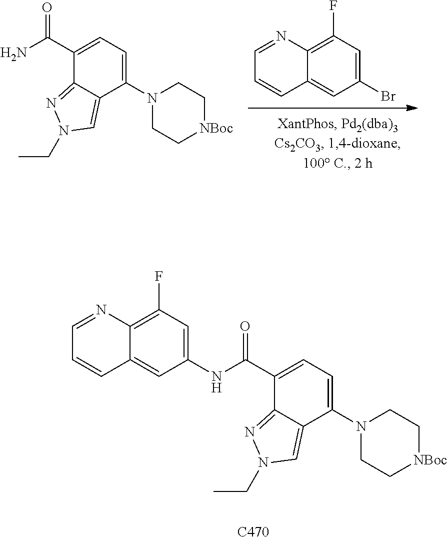

To a stirred mixture of tert-butyl 4-(7-carbamoyl-2-ethylindazol-4-yl)piperazine-1-carboxylate (100 mg, 0.268 mmol, 1 equiv) and 6-bromo-8-fluoroquinoline (78.7 mg, 0.348 mmol, 1.3 equiv) in dioxane (2 mL) were added Cs₂CO₃ (261.7 mg, 0.804 mmol, 3 equiv) and XantPhos (31 mg, 0.054 mmol, 0.2 equiv) and Pd₂(dba)₃ (24.5 mg, 0.027 mmol, 0.1 equiv) at room temperature under nitrogen atmosphere. The resulting mixture was stirred for 2 h at 100° C. under nitrogen atmosphere. The mixture was allowed to cool down to room temperature. The resulting mixture was diluted with water (5 mL). The resulting mixture was extracted with EtOAc (3×5 mL). The combined organic layers were washed with water (3×5 mL), dried over anhydrous Na₂SO₄. After filtration, the filtrate was concentrated under reduced pressure. The residue was purified by silica gel column chromatography, eluted with PE/EA (1:1) to afford tert-butyl 4-{2-ethyl-7-[(8-fluoroquinolin-6-yl)carbamoyl]indazol-4-yl}piperazine-1-carboxylate (100 mg, 72%) as a solid. LCMS (ES, m/z): 519 [M+H]⁺

### Synthesis of Compound 575

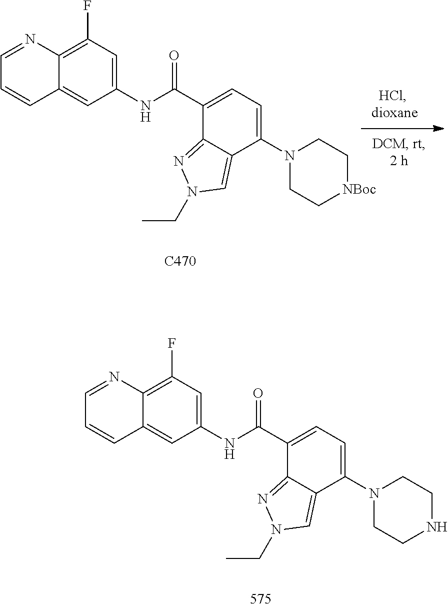

To a stirred mixture of tert-butyl 4-[7-({8-chloro-2-methylimidazo[1,2-a]pyrazin-6-yl}carbamoyl)-2-ethylindazol-4-yl]piperazine-1-carboxylate (60 mg, 0.111 mmol, 1 equiv) in DCM (1 mL) was added HCl(gas) in 1,4-dioxane (0.25 mL) dropwise at room temperature. The resulting mixture was stirred for 2 h at room temperature. The resulting mixture was concentrated under vacuum. The crude product was purified by Prep-HPLC (Condition 10, Gradient 2) to afford N-{8-chloro-2-methylimidazo[1,2-a]pyrazin-6-yl}-2-ethyl-4-(piperazin-1-yl) indazole-7-carboxamide (15.8 mg, 32%) as a solid. LCMS (ES, m/z): 419 [M+H]^{+ 1}H NMR (400 MHz, DMSO-d₆) δ 11.56 (s, 1H), 8.85 (s, 2H), 8.43 (d, J=8.4 Hz, 1H), 8.28 (d, J=2.2 Hz, 1H), 8.10-8.01 (m, 2H), 7.61 (dd, J=8.4, 4.2 Hz, 1H), 6.51 (d, J=8.1 Hz, 1H), 4.63 (q, J=7.3 Hz, 2H), 3.37 (t, J=4.9 Hz, 4H), 2.92 (t, J=4.9 Hz, 4H), 1.66 (t, J=7.3 Hz, 3H).

### Example 285: Synthesis of Compound 576

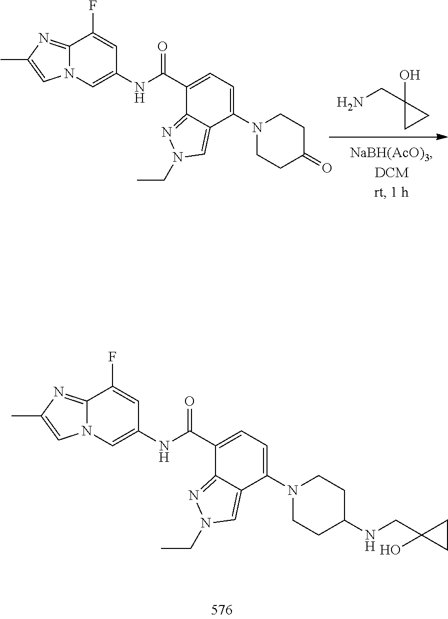 [SMILES: CCn1cc2c(N3CCC(=O)CC3)ccc(C(=O)Nc3cc(F)c4nc(C)cn4c3)c2n1 ; NCC1(O)CC1 ; CCn1cc2c(N3CCC(NCC4(O)CC4)CC3)ccc(C(=O)Nc3cc(F)c4nc(C)cn4c3)c2n1]

To a stirred mixture of 2-ethyl-N-{8-fluoro-2-methylimidazo[1,2-a]pyridin-6-yl}-4-(4-oxopiperidin-1-yl) indazole-7-carboxamide (100.0 mg, 0.230 mmol, 1.0 equiv) and 1-(aminomethyl)cyclopropan-1-ol (30.0 mg, 0.345 mmol, 1.5 equiv) in DCM (1 mL) was added NaBH(AcO)₃ (97.6 mg, 0.460 mmol, 2.0 equiv) in portions at room temperature under nitrogen atmosphere. The resulting mixture was stirred for 1 h at room temperature. The reaction was quenched with water at room temperature. The resulting mixture was concentrated under reduced pressure. The crude product was purified by Prep-HPLC (Condition 10, Gradient 2) to afford 2-ethyl-N-{8-fluoro-2-methylimidazo[1,2-a]pyridin-6-yl}-4-(4-{[(1-hydroxycyclopropyl)methyl]amino}piperidin-1-yl) indazole-7-carboxamide (41 mg, 35%) as a solid. LCMS (ES, m/z): 506 [M+H]^{+ 1}H NMR (400 MHz, DMSO-d₆) δ 11.10 (s, 1H), 9.21 (d, J=1.7 Hz, 1H), 8.80 (s, 1H), 7.97 (d, J=8.1 Hz, 1H), 7.90 (d, J=3.1 Hz, 1H), 7.29 (dd, J=12.3, 1.7 Hz, 1H), 6.49 (d, J=8.1 Hz, 1H), 5.12 (s, 1H), 4.60 (q, J=7.3 Hz, 2H), 3.94-3.83 (m, 2H), 3.13-2.97 (m, 2H), 2.75 (dq, J=9.1, 4.7, 3.8 Hz, 1H), 2.67 (s, 2H), 2.35 (s, 3H), 1.97 (dd, J=13.3, 4.0 Hz, 2H), 1.62 (t, J=7.2 Hz, 4H), 1.54-1.37 (m, 2H), 0.54 (q, J=4.5, 4.0 Hz, 2H), 0.44 (q, J=4.9, 4.5 Hz, 2H).

### Example 286: Synthesis of Compound 577

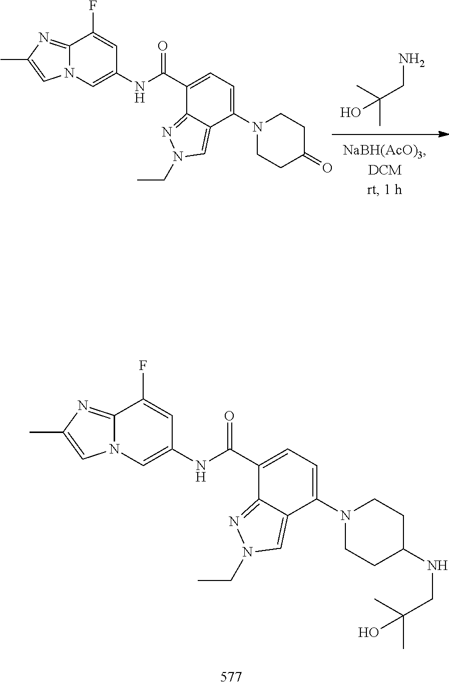

To a stirred mixture of 2-ethyl-N-{8-fluoro-2-methylimidazo[1,2-a]pyridin-6-yl}-4-(4-oxopiperidin-1-yl) indazole-7-carboxamide (100 mg, 0.230 mmol, 1 equiv) and 1-amino-2-methylpropan-2-ol (30.7 mg, 0.345 mmol, 1.5 equiv) in DCM (1 mL) was added NaBH(AcO)₃ (97.5 mg, 0.460 mmol, 2 equiv) in portions at room temperature under nitrogen atmosphere. The resulting mixture was stirred for 1 h at room temperature. The reaction was quenched with water at room temperature. The resulting mixture was concentrated under reduced pressure. The crude product was purified by Prep-HPLC (Condition 10, Gradient 3) to afford 2-ethyl-N-{8-fluoro-2-methylimidazo[1,2-a]pyridin-6-yl}-4-{4-[(2-hydroxy-2-methylpropyl)amino]piperidin-1-yl}indazole-7-carboxamide (50.1 mg, 47%) as a solid. LCMS (ES, m/z): 506 [M+H]^{+ 1}H NMR (400 MHz, DMSO-d₆) δ 11.11 (s, 1H), 9.21 (d, J=1.7 Hz, 1H), 8.80 (s, 1H), 7.97 (d, J=8.1 Hz, 1H), 7.91 (d, J=3.1 Hz, 1H), 7.30 (dd, J=12.3, 1.6 Hz, 1H), 6.49 (d, J=8.2 Hz, 1H), 4.60 (q, J=7.3 Hz, 2H), 4.17 (s, 1H), 3.88 (dd, J=10.7, 6.6 Hz, 2H), 3.07 (t, J=11.8 Hz, 2H), 2.66 (d, J=11.6 Hz, 1H), 2.47 (s, 2H), 2.35 (s, 3H), 1.97 (d, J=12.3 Hz, 2H), 1.63 (d, J=7.3 Hz, 3H), 1.57-1.40 (m, 3H), 1.11 (s, 6H).

### Example 287: Synthesis of Compound 579

### Synthesis of Intermediate C471

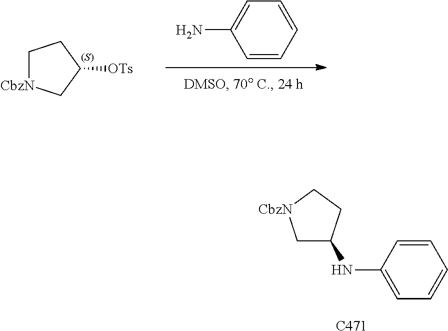

A solution of benzyl (3S)-3-[(4-methyl benzenesulfonyl)oxy] pyrrolidine-1-carboxylate (1 g, 2.664 mmol, 1 equiv) in DMSO (5 mL) was added DIEA (1.72 g, 13.320 mmol, 5 equiv) and aniline (1.24 g, 13.320 mmol, 5 equiv). The mixture was stirred for 24 h at 70° C. The resulting mixture was washed with 3×10 mL of water and extracted with EtOAc (2×20 mL), dried over anhydrous Na₂SO₄. After filtration, the filtrate was concentrated under reduced pressure. The residue was purified by silica gel column chromatography, eluted with PE/EA (15%) to afford benzyl (3R)-3-(phenylamino) pyrrolidine-1-carboxylate (530 mg, 67%) as an oil. LCMS (ES, m/z): 297 [M+H]⁺

### Synthesis of Intermediate C472

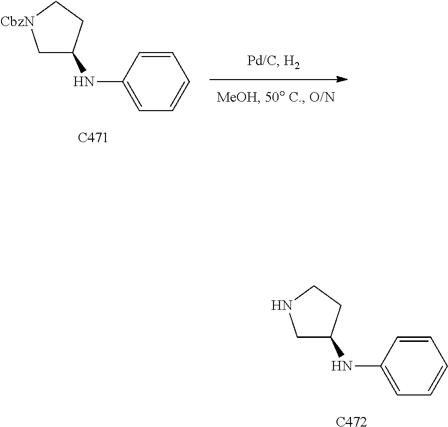 [SMILES: O=C(OCc1ccccc1)N1CC[C@@H](Nc2ccccc2)C1 ; "C471" ; c1ccc(N[C@@H]2CCNC2)cc1 ; "C472"]

A solution of benzyl (3R)-3-(phenylamino) pyrrolidine-1-carboxylate (120 mg, 0.405 mmol, 1 equiv) in MeOH (10 mL) was added Pd/C (20 mg, 10%). The mixture was stirred for overnight at 50° C. under hydrogen atmosphere. The resulting mixture was filtered and the filtrate was concentrated under reduced pressure to afford (3R)—N-phenylpyrrolidin-3-amine (60 mg, 91.34%) as an oil. LCMS (ES, m/z): 163 [M+H]⁺

### Synthesis of Compound 579

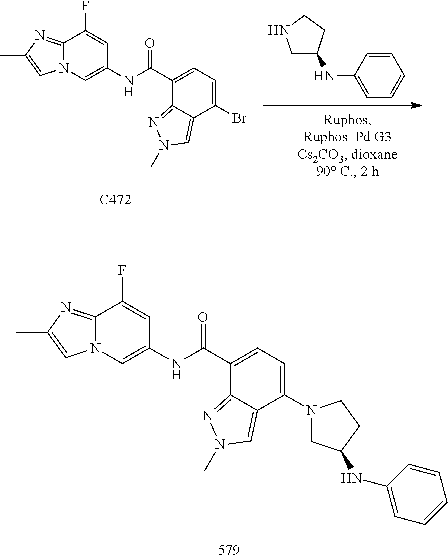 [SMILES: Cc1cn2cc(NC(=O)c3ccc(Br)c4cn(C)nc34)cc(F)c2n1 ; c1ccc(N[C@@H]2CCNC2)cc1 ; "C472" ; Cc1cn2cc(NC(=O)c3ccc(N4CC[C@@H](Nc5ccccc5)C4)c4cn(C)nc34)cc(F)c2n1 ; "579"]

A solution of 4-bromo-N-{8-fluoro-2-methylimidazo[1,2-a] pyridin-6-yl}-2-methylindazole-7-carboxamide (60 mg, 0.149 mmol, 1.0 equiv) in 1,4-dioxane (0.5 mL) was added (3R)—N-phenylpyrrolidin-3-amine (36.30 mg, 0.223 mmol, 1.5 equiv), RuPhos (13.92 mg, 0.030 mmol, 0.2 equiv) and RuPhos Palladacycle Gen.3 (12.48 mg, 0.015 mmol, 0.1 equiv) under nitrogen atmosphere. The reaction was stirred for 2 h at 90° C. The resulting mixture was concentrated under vacuum. The residue was purified by silica gel column chromatography, eluted with PE/EA (60%) to afford the crude product. The crude product was purified by Prep-HPLC (Condition 10, Gradient 2) to afford N-{8-fluoro-2-methylimidazo[1,2-a]pyridin-6-yl}-2-methyl-4-[(3R)-3-(phenylamino) pyrrolidin-1-yl] indazole-7-carboxamide (5 mg, 7%) as a solid. LCMS (ES, m/z): 460 [M+H]^{+ 1}H NMR (400 MHz, DMSO-d₆) δ 11.02 (s, 1H), 9.20 (d, J=1.6 Hz, 1H), 8.85 (s, 1H), 7.94 (d, J=8.2 Hz, 1H), 7.91-7.86 (m, 1H), 7.32 (dd, J=12.3, 1.6 Hz, 1H), 7.11 (t, J=7.7 Hz, 2H), 6.67 (d, J=7.9 Hz, 2H), 6.57 (t, J=7.3 Hz, 1H), 6.07 (d, J=8.4 Hz, 1H), 5.96 (d, J=6.6 Hz, 1H), 4.27 (s, 3H), 4.22 (s, 1H), 4.01 (d, J=7.1 Hz, 1H), 3.84 (d, J=9.0 Hz, 1H), 3.75 (s, 1H), 3.53 (d, J=11.6 Hz, 1H), 2.35 (s, 3H), 2.14-1.92 (m, 2H), 1.30-1.24 (m, 5H), 0.85 (d, J=7.1 Hz, 1H).

### Example 288: Synthesis of Compound 580

### Synthesis of Intermediate C473

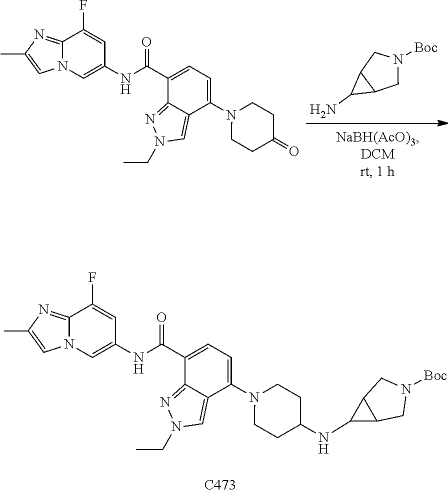 [SMILES: CCn1cc2c(N3CCC(=O)CC3)ccc(C(=O)Nc3cc(F)c4nc(C)cn4c3)c2n1 ; CC(C)(C)OC(=O)N1CC2C(N)C2C1 ; CCn1cc2c(N3CCC(NC4C5CN(C(=O)OC(C)(C)C)CC54)CC3)ccc(C(=O)Nc3cc(F)c4nc(C)cn4c3)c2n1 ; "C473"]

To a stirred mixture of 2-ethyl-N-{8-fluoro-2-methylimidazo[1,2-a]pyridin-6-yl}-4-(4-oxopiperidin-1-yl) indazole-7-carboxamide (100.0 mg, 0.230 mmol, 1.0 equiv) and tert-butyl 6-amino-3-azabicyclo[3.1.0]hexane-3-carboxylate (68.4 mg, 0.345 mmol, 1.5 equiv) in DCM (1 mL) was added NaBH(AcO)₃ (97.6 mg, 0.460 mmol, 2.0 equiv) in portions at room temperature under nitrogen atmosphere. The resulting mixture was stirred for 1 h at room temperature. The reaction was quenched with water at room temperature. The resulting mixture was extracted with CH₂Cl₂ (3×10 mL). The combined organic layers were washed with brine (1×10 mL), dried over anhydrous Na₂SO₄. After filtration, the filtrate was concentrated under reduced pressure to afford tert-butyl 6-({1-[2-ethyl-7-({8-fluoro-2-methylimidazo[1,2-a]pyridin-6-yl}carbamoyl) indazol-4-yl]piperidin-4-yl}amino)-3-azabicyclo[3.1.0]hexane-3-carboxylate (113.8 mg, 80%) as a solid. LCMS (ES, m/z): 617 [M+H]⁺

### Synthesis of Compound 580

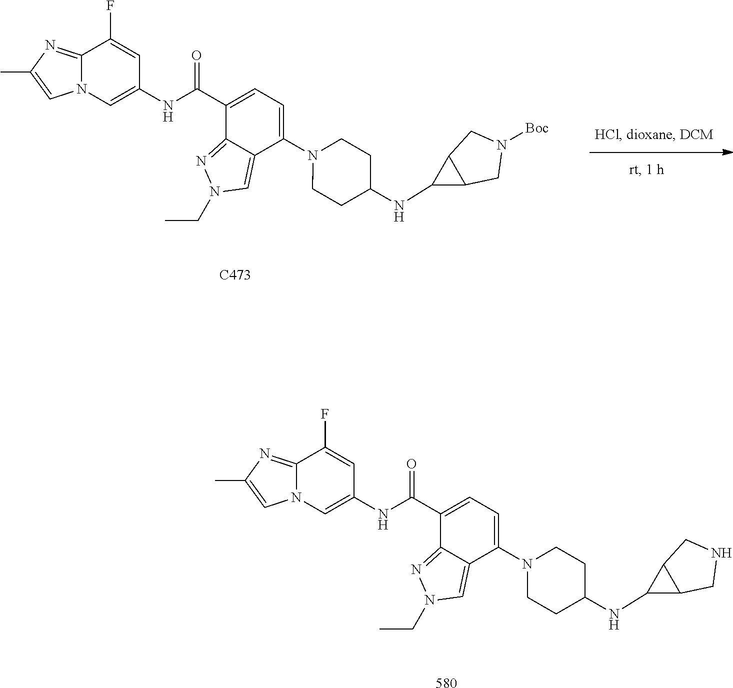 [SMILES: CCn1cc2c(N3CCC(NC4C5CN(C(=O)OC(C)(C)C)CC54)CC3)ccc(C(=O)Nc3cc(F)c4nc(C)cn4c3)c2n1 ; "C473" ; CCn1cc2c(N3CCC(NC4C5CNCC54)CC3)ccc(C(=O)Nc3cc(F)c4nc(C)cn4c3)c2n1 ; "580"]

A solution of tert-butyl 6-({1-[2-ethyl-7-({8-fluoro-2-methylimidazo[1,2-a]pyridin-6-yl}carbamoyl) indazol-4-yl]piperidin-4-yl}amino)-3-azabicyclo[3.1.0]hexane-3-carboxylate (113 mg, 0.183 mmol, 1.0 equiv) in DCM (0.9 mL) was treated with HCl(gas) in 1,4-dioxane (0.3 mL) at room temperature. The resulting mixture was stirred for 1 h at room temperature. The resulting mixture was concentrated under reduced pressure. The crude product was purified by Prep-HPLC (Condition 10, Gradient 2) to afford 4-(4-{3-azabicyclo[3.1.0]hexan-6-ylamino}piperidin-1-yl)-2-ethyl-N-{8-fluoro-2-methylimidazo[1,2-a]pyridin-6-yl}indazole-7-carboxamide; bis(trifluoroacetic acid) (60 mg, 44%) as a solid. LCMS (ES, m/z): 517 [M+H]^{+ 1}H NMR (400 MHz, DMSO-d₆) δ 11.23 (s, 1H), 9.45-9.30 (m, 4H), 8.88 (s, 1H), 8.72 (s, 1H), 8.08 (s, 1H), 8.02 (d, J=8.0 Hz, 1H), 7.65 (d, J=11.9 Hz, 1H), 6.58 (d, J=8.2 Hz, 1H), 4.62 (q, J=7.3 Hz, 2H), 4.08 (d, J=12.8 Hz, 2H), 3.46 (d, J=5.9 Hz, 5H), 3.06 (t, J=12.4 Hz, 2H), 2.81 (s, 1H), 2.42 (s, 3H), 2.33 (s, 2H), 2.22-2.09 (m, 2H), 1.76 (td, J=13.7, 9.9 Hz, 2H), 1.64 (t, J=7.2 Hz, 3H).

### Example 289: Synthesis of Compound 581

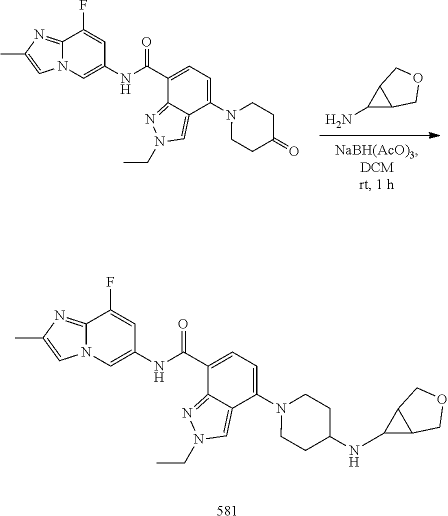 [SMILES: CCn1cc2c(N3CCC(=O)CC3)ccc(C(=O)Nc3cc(F)c4nc(C)cn4c3)c2n1 ; NC1C2COCC12 ; CCn1cc2c(N3CCC(NC4C5COCC54)CC3)ccc(C(=O)Nc3cc(F)c4nc(C)cn4c3)c2n1 ; "581"]

To a stirred mixture of 2-ethyl-N-{8-fluoro-2-methylimidazo[1,2-a]pyridin-6-yl}-4-(4-oxopiperidin-1-yl) indazole-7-carboxamide (100.0 mg, 0.230 mmol, 1.0 equiv) and 1-(aminomethyl)cyclopropan-1-ol (30.0 mg, 0.345 mmol, 1.5 equiv) in DCM (1 mL) was added NaBH(AcO)₃ (97.6 mg, 0.460 mmol, 2.0 equiv) in portions at room temperature under nitrogen atmosphere. The resulting mixture was stirred for 1 h at room temperature. The reaction was quenched with water at room temperature. The resulting mixture was concentrated under reduced pressure. The crude product was purified by Prep-HPLC (Condition 10, Gradient 2) to afford 2-ethyl-N-{8-fluoro-2-methylimidazo[1,2-a]pyridin-6-yl}-4-(4-{3-oxabicyclo[3.1.0]hexan-6-ylamino}piperidin-1-yl) indazole-7-carboxamide (16.6 mg, 14%) as a solid. LCMS (ES, m/z): 518 [M+H]^{+ 1}H NMR (400 MHz, DMSO-d₆) δ 11.10 (s, 1H), 9.21 (d, J=1.7 Hz, 1H), 8.79 (s, 1H), 7.97 (d, J=8.1 Hz, 1H), 7.90 (d, J=3.0 Hz, 1H), 7.29 (dd, J=12.3, 1.7 Hz, 1H), 6.48 (d, J=8.2 Hz, 1H), 4.59 (q, J=7.3 Hz, 2H), 3.96-3.82 (m, 2H), 3.75 (d, J=8.2 Hz, 2H), 3.59 (d, J=8.2 Hz, 2H), 3.12-2.98 (m, 2H), 2.75 (tt, J=9.5, 4.0 Hz, 1H), 2.35 (s, 3H), 2.26 (s, 1H), 2.04-1.93 (m, 2H), 1.91 (d, J=2.3 Hz, 1H), 1.66-1.54 (m, 5H), 1.55-1.36 (m, 2H).

### Example 290: Synthesis of Compound 582

### Synthesis of Intermediate C474

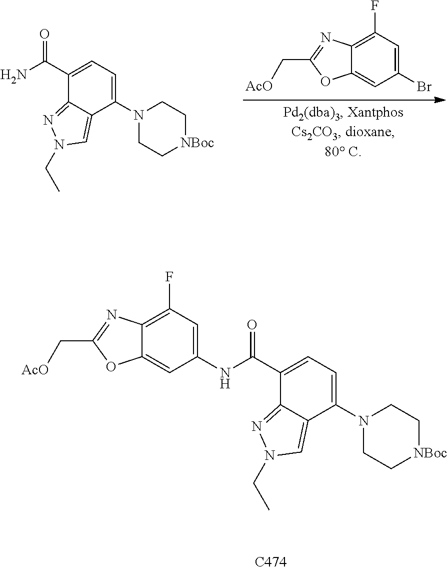 [SMILES: CCn1cc2c(N3CCN(C(=O)OC(C)(C)C)CC3)ccc(C(N)=O)c2n1 ; CC(=O)OCc1nc2c(F)cc(Br)cc2o1 ; CCn1cc2c(N3CCN(C(=O)OC(C)(C)C)CC3)ccc(C(=O)Nc3cc(F)c4nc(COC(C)=O)oc4c3)c2n1 ; "C474"]

To a solution of tert-butyl 4-(7-carbamoyl-2-ethylindazol-4-yl)piperazine-1-carboxylate (100 mg, 0.268 mmol, 1 equiv) and (6-bromo-4-fluoro-1,3-benzoxazol-2-yl)methyl acetate (92.5 mg, 0.322 mmol, 1.2 equiv) in dioxane (2 mL) were added Cs₂CO₃ (261.7 mg, 0.804 mmol, 3.0 equiv), XantPhos (30.9 mg, 0.054 mmol, 0.2 equiv) and Pd₂(dba)₃·CHCl₃ (27.7 mg, 0.027 mmol, 0.1 equiv). After stirring for 1 h at 80° C. under a nitrogen atmosphere, the resulting mixture was concentrated under reduced pressure. The residue was purified by silica gel column chromatography, eluted with PE:EA (2:1) to afford tert-butyl 4-[7-({2-[(acetyloxy)methyl]-4-fluoro-1,3-benzoxazol-6-yl}carbamoyl)-2-ethylindazol-4-yl]piperazine-1-carboxylate (120 mg, 77%) as a solid. LCMS (ES, m/z): 581 [M+H]⁺

### Synthesis of Intermediate C475

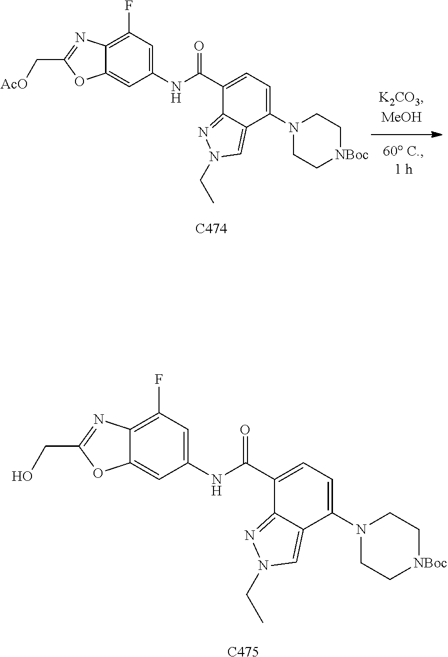

To a stirred solution of tert-butyl 4-[7-({2-[(acetyloxy)methyl]-4-fluoro-1,3-benzoxazol-6-yl}carbamoyl)-2-ethylindazol-4-yl]piperazine-1-carboxylate (120 mg, 0.207 mmol, 1 equiv) in MeOH (2 mL) was added K₂CO₃ (171 mg, 1.242 mmol, 6.0 equiv) at room temperature. The resulting mixture was stirred for 1 h at 60° C. The resulting mixture was concentrated under reduced pressure. The residue was purified by reverse flash chromatography (Condition 5, Gradient 3) to afford tert-butyl 4-(2-ethyl-7-{[4-fluoro-2-(hydroxymethyl)-1,3-benzoxazol-6-yl]carbamoyl}indazol-4-yl)piperazine-1-carboxylate (100 mg, 90%) as a solid. LCMS (ES, m/z): 539 [M+H]⁺

### Synthesis of Compound 582

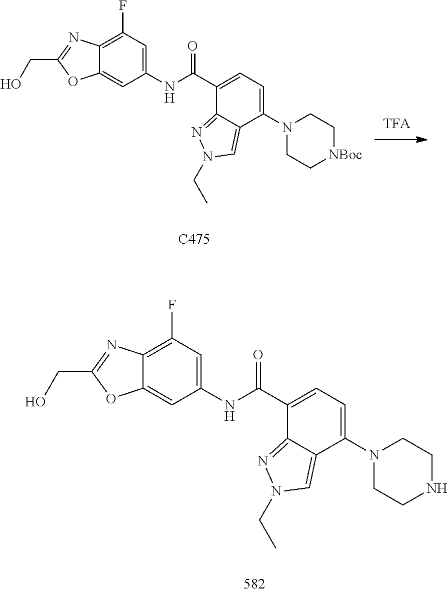 [SMILES: CCn1cc2c(N3CCN(C(=O)OC(C)(C)C)CC3)ccc(C(=O)Nc3cc(F)c4nc(CO)oc4c3)c2n1 ; "C475" ; CCn1cc2c(N3CCNCC3)ccc(C(=O)Nc3cc(F)c4nc(CO)oc4c3)c2n1 ; "582"]

A solution of tert-butyl 4-(2-ethyl-7-{[4-fluoro-2-(hydroxymethyl)-1,3-benzoxazol-6-yl]carbamoyl}indazol-4-yl)piperazine-1-carboxylate (50 mg, 0.093 mmol, 1 equiv) and TFA (0.2 mL) in DCM (2 mL) was stirred for 1 h at room temperature. The mixture was basified to pH 8 with NH₃ (g) in MeOH. The resulting mixture was concentrated under reduced pressure. The residue was purified by reverse flash chromatography (Condition 3, Gradient 5) to afford 2-ethyl-N-[4-fluoro-2-(hydroxymethyl)-1,3-benzoxazol-6-yl]-4-(piperazin-1-yl) indazole-7-carboxamide (20 mg, 49%) as a solid. LCMS (ES, m/z): 439 [M+H]^{+ 1}H NMR (300 MHz, DMSO-d₆) δ 11.53 (s, 1H), 8.84 (s, 1H), 8.18 (d, J=1.7 Hz, 1H), 8.01 (d, J=8.1 Hz, 1H), 7.65 (dd, J=12.0, 1.7 Hz, 1H), 6.50 (d, J=8.2 Hz, 1H), 5.94 (s, 1H), 4.71 (d, J=4.4 Hz, 2H), 4.61 (q, J=7.3 Hz, 2H), 3.37-3.32 (m, 4H), 2.92 (t, J=5.0 Hz, 4H), 1.63 (t, J=7.3 Hz,

### Example 291: Synthesis of Compound 584

### Synthesis of Intermediate C476

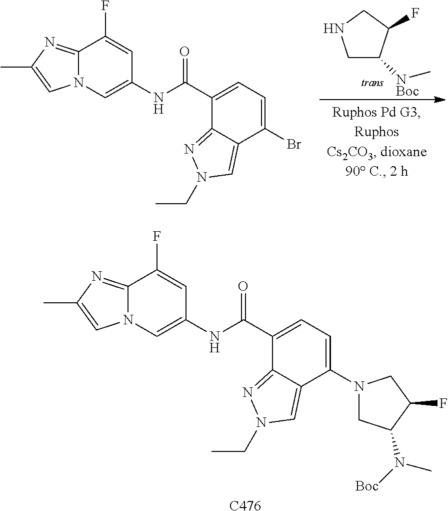

To a stirred mixture of 4-bromo-2-ethyl-N-{8-fluoro-2-methylimidazo[1,2-a]pyridin-6-yl}indazole-7-carboxamide (200 mg, 0.480 mmol, 1 equiv), trans-tert-butyl N-[(3R,4R)-4-fluoropyrrolidin-3-yl]-N-methylcarbamate (126 mg, 0.576 mmol, 1.2 equiv) and Cs₂CO₃ (313 mg, 0.960 mmol, 2 equiv) in 1,4-dioxane (2 mL) were added RuPhos Palladacycle Gen.3 (40 mg, 0.048 mmol, 0.1 equiv) and Ruphos (22 mg, 0.048 mmol, 0.1 equiv) at room temperature under nitrogen atmosphere. The resulting mixture was stirred for 2 h at 90° C. under nitrogen atmosphere. The reaction mixture was concentrated under reduced pressure. The residue was purified by silica gel column chromatography, eluted with PE:EA (1:1) to afford trans-tert-butyl N-[(3R,4R)-1-[2-ethyl-7-({8-fluoro-2-methylimidazo[1,2-a]pyridin-6-yl}carbamoyl) indazol-4-yl]-4-fluoropyrrolidin-3-yl]-N-methylcarbamate (108 mg, 41%) as a solid. LCMS (ES, m/z): 554 [M+H]⁺

### Synthesis of Compound 584

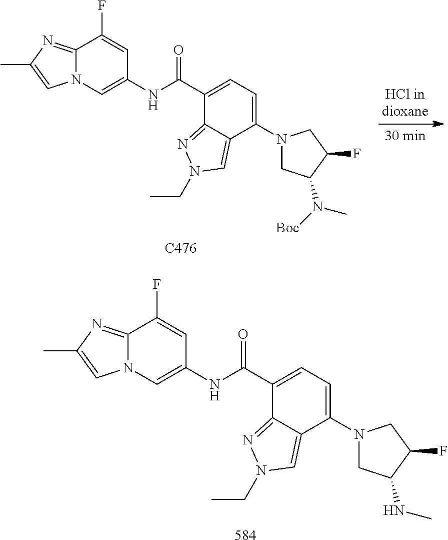

To a stirred solution of trans-tert-butyl N-[(3R,4R)-1-[2-ethyl-7-({8-fluoro-2-methylimidazo[1,2-a]pyridin-6-yl}carbamoyl) indazol-4-yl]-4-fluoropyrrolidin-3-yl]-N-methylcarbamate (100 mg, 0.181 mmol, 1 equiv) in DCM (2 mL) was added 4M HCl(gas) in 1,4-dioxane (0.2 mL) at room temperature under nitrogen atmosphere. The resulting mixture was stirred for 30 min at room temperature. The resulting mixture was concentrated under reduced pressure. The residue was purified by reverse flash chromatography (Condition 3, Gradient 1) to afford 2-ethyl-N-{8-fluoro-2-methylimidazo[1,2-a]pyridin-6-yl}-4-[(3R,4R)-3-fluoro-4-(methylamino)pyrrolidin-1-yl]indazole-7-carboxamide (6.7 mg, 8%) as a solid. LCMS (ES, m/z): 454 [M+H]^{+ 1}H NMR (300 MHz, DMSO-d₆) δ 11.06 (s, 1H), 9.20 (d, J=1.6 Hz, 1H), 8.89 (s, 1H), 7.95 (d, J=8.2 Hz, 1H), 7.91-7.87 (m, 1H), 7.29 (dd, J=12.4, 1.7 Hz, 1H), 6.08 (d, J=8.4 Hz, 1H), 5.27 (d, J=50.8 Hz, 1H), 4.58 (q, J=7.3 Hz, 2H), 4.20-3.78 (m, 3H), 3.62 (d, J=10.9 Hz, 1H), 2.40-2.32 (m, 6H), 1.62 (t, J=7.3 Hz, 3H).

### Example 292: Synthesis of Compound 585

### Synthesis of Intermediate C477

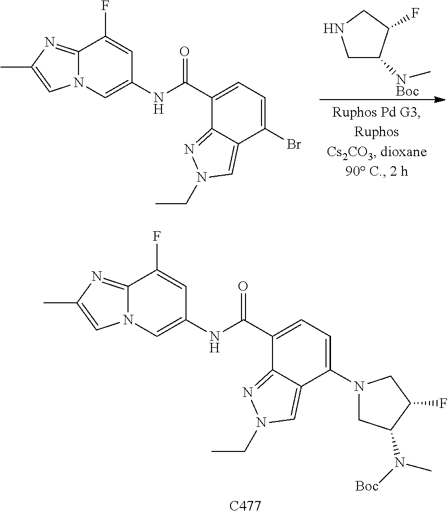

To a stirred mixture of 4-bromo-2-ethyl-N-{8-fluoro-2-methylimidazo[1,2-a]pyridin-6-yl}indazole-7-carboxamide (200 mg, 0.480 mmol, 1 equiv), cis-tert-butyl N-[(3R,4S)-4-fluoropyrrolidin-3-yl]-N-methylcarbamate (125.85 mg, 0.576 mmol, 1.2 equiv) and Cs₂CO₃ (313.10 mg, 0.960 mmol, 2 equiv) in 1,4-dioxane (5 mL) were added RuPhos Palladacycle Gen.3 (40.19 mg, 0.048 mmol, 0.1 equiv) and Ruphos (22.42 mg, 0.048 mmol, 0.1 equiv) at room temperature under nitrogen atmosphere. The resulting mixture was stirred for 2 h at 90° C. under nitrogen atmosphere. The reaction mixture was concentrated under reduced pressure. The residue was purified by silica gel column chromatography, eluted with PE:EA (1:1) to afford cis-tert-butyl N-[(3R,4S)-1-[2-ethyl-7-({8-fluoro-2-methylimidazo[1,2-a]pyridin-6-yl}carbamoyl) indazol-4-yl]-4-fluoropyrrolidin-3-yl]-N-methylcarbamate (110 mg, 41%) as a solid. LCMS (ES, m/z): 554 [M+H]⁺

### Synthesis of Compound 585

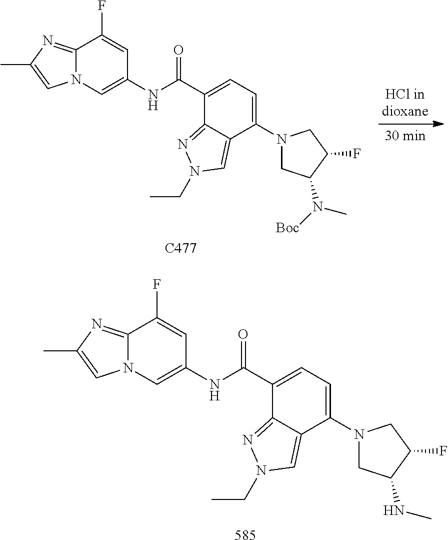

To a stirred solution of cis-tert-butyl N-[(3R,4S)-1-[2-ethyl-7-({8-fluoro-2-methylimidazo[1,2-a]pyridin-6-yl}carbamoyl) indazol-4-yl]-4-fluoropyrrolidin-3-yl]-N-methylcarbamate (100 mg, 0.181 mmol, 1 equiv) in DCM (2 mL) was added 4M HCl(gas) in 1,4-dioxane (0.2 mL) at room temperature. The resulting mixture was stirred for 30 min at room temperature. The resulting mixture was concentrated under reduced pressure. The residue was purified by reverse flash chromatography (Condition 6, Gradient 3) to afford cis-2-ethyl-N-(8-fluoro-2-methylimidazo[1,2-a]pyridin-6-yl)-4-((3S,4R)-3-fluoro-4-(methylamino)pyrrolidin-1-yl)-2H-indazole-7-carboxamide bis(2,2,2-trifluoroacetate) (55 mg, 39%) as a solid. LCMS (ES, m/z): 454 [M+H]^{+ 1}H NMR (400 MHz, DMSO-d₆) δ 11.27 (d, J=2.8 Hz, 1H), 10.02 (s, 1H), 9.44 (d, J=6.8 Hz, 1H), 8.86-8.73 (m, 1H), 8.06-7.95 (m, 2H), 7.57 (dd, J=23.2, 13.1 Hz, 1H), 6.04 (q, J=8.7, 6.9 Hz, 1H), 5.66 (d, J=53.9 Hz, 1H), 4.58 (q, J=7.8 Hz, 2H), 4.28-3.97 (m, 4H), 3.93-3.80 (m, 1H), 2.82 (s, 3H), 1.66 (td, J=7.3, 2.3 Hz, 3H).

### Example 293: Synthesis of Compound 587

### Synthesis of Intermediate C478

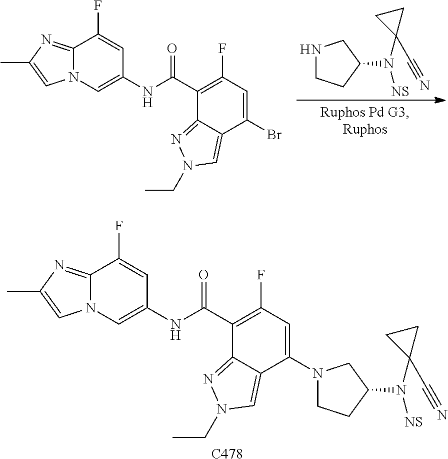

To a solution of 4-bromo-6-fluoro-N-{8-fluoro-2-methylimidazo[1,2-a]pyridin-6-yl}-2-methylindazole-7-carboxamide (150 mg, 0.357 mmol, 1 equiv) and N-(1-cyanocyclopropyl)-4-nitro-N-[(3R)-pyrrolidin-3-yl]benzenesulfonamide (144.0 mg, 0.428 mmol, 1.2 equiv) in dioxane (3 mL) were added RuPhos (33.3 mg, 0.071 mmol, 0.2 equiv), Cs₂CO₃ (348.9 mg, 1.071 mmol, 3.0 equiv) and RuPhos Palladacycle Gen.3 (29.8 mg, 0.036 mmol, 0.1 equiv). After stirring for 3 h at 80° C. under a nitrogen atmosphere, the resulting mixture was concentrated under reduced pressure. The residue was purified by reverse flash chromatography (Condition 5, Gradient 2) to afford 4-[(3R)-3-[N-(1-cyanocyclopropyl)4-nitrobenzenesulfonamido]pyrrolidin-1-yl]-6-fluoro-N-{8-fluoro-2-methylimidazo[1,2-a]pyridin-6-yl}-2-methylindazole-7-carboxamide (120 mg, 50%) as a solid. LCMS (ES, m/z): 676 [M+H]⁺

### Synthesis of Compound 587

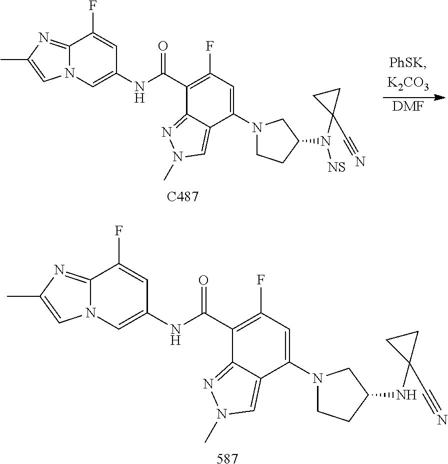

To a stirred solution of 4-[(3R)-3-[N-(1-cyanocyclopropyl)4-nitrobenzenesulfonamido]pyrrolidin-1-yl]-6-fluoro-N-{8-fluoro-2-methylimidazo[1,2-a]pyridin-6-yl}-2-methylindazole-7-carboxamide (70 mg, 0.104 mmol, 1 equiv) and (phenylsulfanyl)potassium (23.0 mg, 0.156 mmol, 1.5 equiv) in DMF (2 mL) was added K₂CO₃ (28.6 mg, 0.208 mmol, 2.0 equiv) at room temperature. The resulting mixture was stirred for 3 h at room temperature. The resulting mixture was diluted with H₂O (5 mL). The resulting mixture was extracted with EA (3×5 mL). The combined organic layers were washed with brine (1×10 mL), dried over anhydrous Na₂SO₄. After filtration, the filtrate was concentrated under reduced pressure. The residue was purified by reverse flash chromatography (Condition 3, Gradient 5) to afford 4-[(3R)-3-[(1-cyanocyclopropyl)amino]pyrrolidin-1-yl]-6-fluoro-N-{8-fluoro-2-methylimidazo[1,2-a]pyridin-6-yl}-2-methylindazole-7-carboxamide (20 mg, 39%) as a solid. LCMS (ES, m/z): 491 [M+H]^{+ 1}H NMR (300 MHz, DMSO-d₆) δ 11.02 (s, 1H), 9.19 (d, J=1.6 Hz, 1H), 8.78 (s, 1H), 7.88 (d, J=3.2 Hz, 1H), 7.25 (dd, J=12.6, 1.7 Hz, 1H), 5.76 (d, J=15.9 Hz, 1H), 4.22 (s, 3H), 3.77 (s, 2H), 3.70 (s, 3H), 3.44 (d, J=9.8 Hz, 1H), 2.37-2.31 (m, 3H), 2.28-2.18 (m, 1H), 2.04 (s, 1H), 1.26 (s, 2H), 1.08-0.95 (m, 2H).

### Example 294: Synthesis of Compound 589

### Synthesis of Intermediate C488

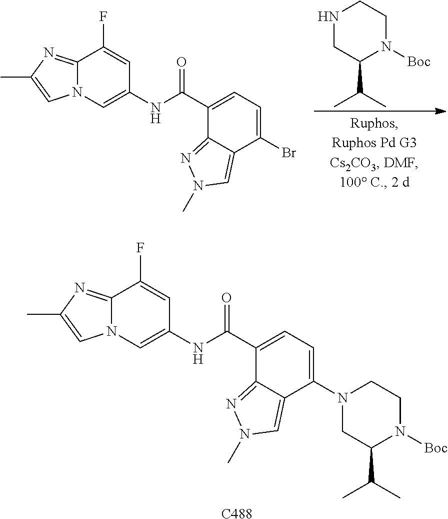 [SMILES: Cc1cn2cc(NC(=O)c3ccc(Br)c4cn(C)nc34)cc(F)c2n1 ; CC(C)[C@H]1CNCCN1C(=O)OC(C)(C)C ; Cc1cn2cc(NC(=O)c3ccc(N4CCN(C(=O)OC(C)(C)C)[C@@H](C(C)C)C4)c4cn(C)nc34)cc(F)c2n1 ; "C488"]

To a solution of 4-bromo-N-{8-fluoro-2-methylimidazo[1,2-a]pyridin-6-yl}-2-methylindazole-7-carboxamide (80.0 mg, 0.199 mmol, 1.0 equiv) and tert-butyl (2S)-2-isopropylpiperazine-1-carboxylate (68.1 mg, 0.298 mmol, 1.5 equiv) in DMF (2 mL) were added Cs₂CO₃ (129.6 mg, 0.398 mmol, 2.0 equiv) and Ruphos (18.5 mg, 0.040 mmol, 0.2 equiv), RuPhos Palladacycle Gen.3 (16.6 mg, 0.020 mmol, 0.1 equiv). After stirring for 2 days at 100° C. under a nitrogen atmosphere, the resulting mixture was concentrated under reduced pressure. The residue was purified by silica gel column chromatography, eluted with PE/EA (1:2) to afford tert-butyl (2S)-4-[7-({8-fluoro-2-methylimidazo[1,2-a]pyridin-6-yl}carbamoyl)-2-methylindazol-4-yl]-2-isopropylpiperazine-1-carboxylate (100 mg, 82%) as a solid. LCMS (ES, m/z): 550 [M+H]⁺

### Synthesis of Compound 589

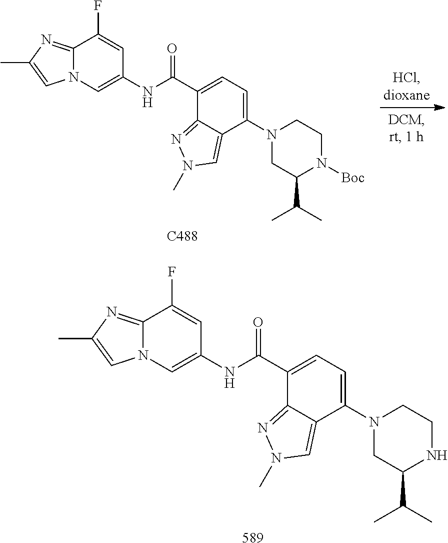

Into a 8 mL vial were added tert-butyl (2S)-4-[7-({8-fluoro-2-methylimidazo[1,2-a]pyridin-6-yl} carbamoyl)-2-methylindazol-4-yl]-2-isopropylpiperazine-1-carboxylate (80.0 mg, 0.146 mmol, 1 equiv), DCM (1 mL) and HCl (gas) in 1,4-dioxane (0.5 mL, 4M) at room temperature. The resulting mixture was stirred for 1 h at room temperature. The resulting mixture was concentrated under reduced pressure. The crude product was purified by Prep-HPLC (Condition 13, Gradient 1) to afford N-{8-fluoro-2-methylimidazo[1,2-a]pyridin-6-yl}-4-[(3S)-3-isopropylpiperazin-1-yl]-2-methylindazole-7-carboxamide (8 mg, 12%) as a solid. LCMS (ES, m/z): 450 [M+H]^{+ 1}H NMR (400 MHz, DMSO-d₆) δ 11.07 (s, 1H), 9.22 (s, 1H), 8.74 (s, 1H), 7.98 (d, J=8.1 Hz, 1H), 7.91 (d, J=3.1 Hz, 1H), 7.35 (d, J=12.2 Hz, 1H), 6.51 (d, J=8.1 Hz, 1H), 4.31 (s, 3H), 3.80 (dd, J=27.2, 10.4 Hz, 2H), 3.05 (d, J=8.8 Hz, 1H), 2.94-2.87 (m, 2H), 2.67 (d, J=10.3 Hz, 1H), 2.36 (s, 3H), 1.67 (dt, J=13.1, 6.7 Hz, 1H), 0.98 (dd, J=6.8, 2.0 Hz, 6H).

### Example 295: Synthesis of Compound 594

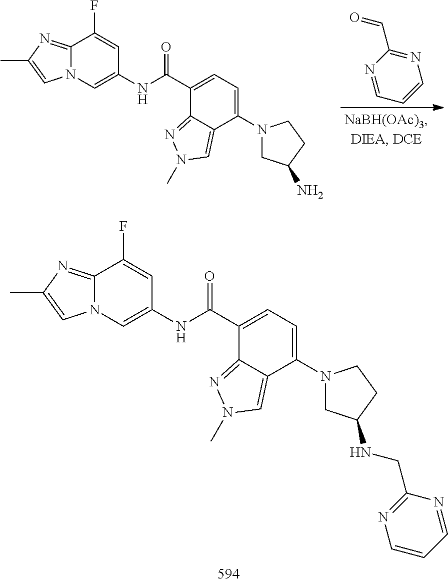 [SMILES: Cc1cn2cc(NC(=O)c3ccc(N4CC[C@@H](N)C4)c4cn(C)nc34)cc(F)c2n1 ; O=Cc1ncccn1 ; Cc1cn2cc(NC(=O)c3ccc(N4CC[C@@H](NCc5ncccn5)C4)c4cn(C)nc34)cc(F)c2n1 ; "594"]

To a stirred mixture of (R)-4-(3-aminopyrrolidin-1-yl)-N-(8-fluoro-2-methylimidazo[1,2-a]pyridin-6-yl)-2-methyl-2H-indazole-7-carboxamide hydrochloride (40 mg, 0.090 mmol, 1 equiv) and DIEA (12 mg, 0.099 mmol, 1.1 equiv) in DCE (2 mL) were added pyrimidine-2-carbaldehyde (10 mg, 0.090 mmol, 1 equiv) and NaBH(OAc)₃ (66 mg, 0.315 mmol, 3.5 equiv) at room temperature. The resulting mixture was stirred for 1.5 h at room temperature. The reaction was quenched with water (2 mL) at 0° C. The resulting mixture was concentrated under vacuum. The residue was purified by reverse flash chromatography (Condition 10, Gradient 1) to afford N-{8-fluoro-2-methylimidazo[1,2-a]pyridin-6-yl}-2-methyl-4-[(3R)-3-[(pyrimidin-2-ylmethyl)amino]pyrrolidin-1-yl]indazole-7-carboxamide (6 mg, 13.33%) as a solid. LCMS (ES, m/z): 500 [M+H]^{+ 1}H NMR (300 MHz, DMSO-d₆) δ 11.02 (s, 1H), 9.20 (d, J=1.6 Hz, 1H), 8.87-8.76 (m, 3H), 7.97-7.85 (m, 2H), 7.41 (t, J=4.9 Hz, 1H), 7.36-7.25 (m, 1H), 6.02 (d, J=8.4 Hz, 1H), 4.27 (s, 3H), 4.03 (s, 2H), 3.76 (d, J=8.8 Hz, 1H), 3.65 (s, 2H), 3.56 (s, 2H), 2.55 (s, 2H), 2.35 (s, 3H), 2.22-2.12 (m, 1H), 2.03 (s, 1H).

### Example 296: Synthesis of Compound 596

### Synthesis of Intermediate C489

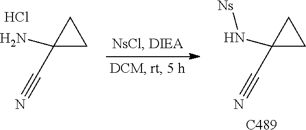

To a stirred solution of 1-aminocyclopropane-1-carbonitrile hydrochloride (2.0 g, 16.869 mmol, 1.0 equiv) and DIEA (4.3 g, 33.738 mmol, 2.0 equiv) in DCM (30 mL) was added 4-nitrobenzene-1-sulfonyl chloride (3.4 g, 15.182 mmol, 0.9 equiv) at room temperature. The resulting mixture was stirred for 5 h at room temperature. The resulting mixture was diluted with deionized water (50 mL). The resulting mixture was extracted with CH₂Cl₂ (2×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (5:1) to afford N-(1-cyanocyclopropyl)-4-nitrobenzenesulfonamide (2.5 g, 50%) as a solid. LCMS (ES, m/z): 268 [M+H]⁺

### Synthesis of Intermediate C490

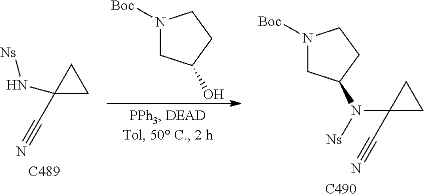

To a stirred solution of N-(1-cyanocyclopropyl)-4-nitrobenzenesulfonamide (1.5 g, 5.613 mmol, 1.0 equiv) and tert-butyl (3S)-3-hydroxypyrrolidine-1-carboxylate (1.4 g, 7.297 mmol, 1.3 equiv) in THF (30 mL) were added DEAD (1.7 g, 10.103 mmol, 1.8 equiv) dropwise at room temperature under nitrogen atmosphere. The resulting mixture was stirred for 2 h at room temperature under nitrogen atmosphere. The resulting mixture was diluted with water (50 mL). The resulting mixture was extracted with EtOAc (2×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (1:1) to afford tert-butyl (3R)-3-[N-(1-cyanocyclopropyl)4-nitrobenzenesulfonamido]pyrrolidine-1-carboxylate (1 g, 38%) as a solid. LCMS (ES, m/z): 437 [M+H]⁺

### Synthesis of Intermediate C491

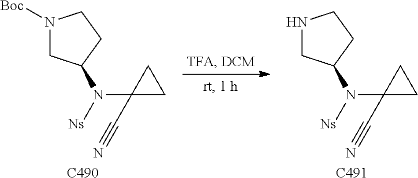

To a stirred solution of tert-butyl (3R)-3-[N-(1-cyanocyclopropyl)4-nitrobenzenesulfonamido] pyrrolidine-1-carboxylate (1.0 g, 2.291 mmol, 1.0 equiv) in DCM (10 mL) was added TFA (5 mL) dropwise at room temperature. The resulting mixture was stirred for 1 h at room temperature. The resulting mixture was concentrated under reduced pressure. The mixture was basified to pH 8 with saturated NaHCO₃ (aq.). The resulting mixture was extracted with EtOAc (2×30 mL). The combined organic layers were washed with brine (1×50 mL), dried over anhydrous Na₂SO₄. After filtration, the filtrate was concentrated under reduced pressure. This resulted in N-(1-cyanocyclopropyl)-4-nitro-N-[(3R)-pyrrolidin-3-yl]benzenesulfonamide (0.7 g, 84%) as a solid. LCMS (ES, m/z): 337 [M+H]⁺

### Synthesis of Intermediate C492

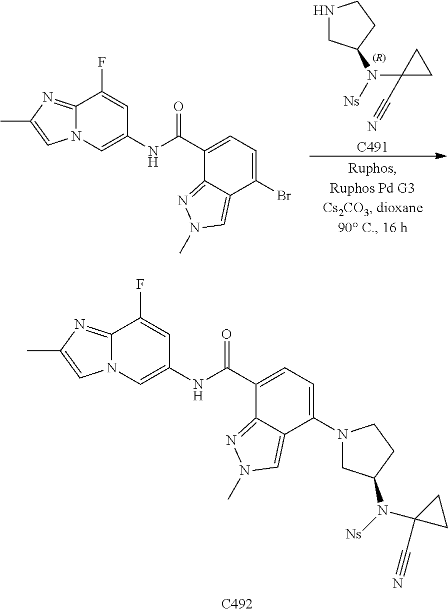

To a solution of 4-bromo-N-{8-fluoro-2-methylimidazo[1,2-a]pyridin-6-yl}-2-methylindazole-7-carboxamide (100.0 mg, 0.249 mmol, 1.0 equiv) and N-(1-cyanocyclopropyl)-4-nitro-N-[(3R)-pyrrolidin-3-yl]benzenesulfonamide (125.4 mg, 0.373 mmol, 1.5 equiv) in DMF (4 mL) were added Cs₂CO₃ (162.01 mg, 0.498 mmol, 2.0 equiv) and Ruphos (23.2 mg, 0.050 mmol, 0.2 equiv), RuPhos Palladacycle Gen.3 (20.8 mg, 0.025 mmol, 0.1 equiv). After stirring for 16 h at 90° C. under a nitrogen atmosphere, the resulting mixture was concentrated under reduced pressure. The residue was purified by silica gel column chromatography, eluted with PE/EA (1:3) to afford 4-[(3R)-3-[N-(1-cyanocyclopropyl)4-nitrobenzenesulfonamido]pyrrolidin-1-yl]-N-{8-fluoro-2-methylimidazo[1,2-a]pyridin-6-yl}-2-methylindazole-7-carboxamide (75 mg, 41%) as a solid. LCMS (ES, m/z): 658 [M+H]⁺

### Synthesis of Compound 596

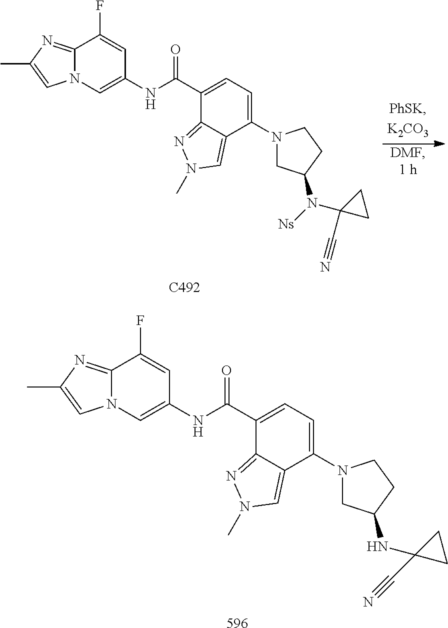

To a stirred solution of 4-[(3R)-3-[N-(1-cyanocyclopropyl)4-nitrobenzenesulfonamido]pyrrolidin-1-yl]-N-{8-fluoro-2-methylimidazo[1,2-a]pyridin-6-yl}-2-methylindazole-7-carboxamide (60.0 mg, 0.091 mmol, 1.0 equiv) and K₂CO₃ (25.2 mg, 0.182 mmol, 2.0 equiv) in DMF (2 mL) was added PhSK (20.3 mg, 0.137 mmol, 1.5 equiv) at room temperature. The resulting mixture was stirred for 1 h at room temperature. The resulting mixture was extracted with EtOAc (2×5 mL). The combined organic layers were washed with brine (1×3 mL), dried over anhydrous Na₂SO₄. After filtration, the filtrate was concentrated under reduced pressure. The crude product was purified by Prep-HPLC (Condition 13, Gradient 1) to afford 4-[(3R)-3-[(1-cyanocyclopropyl)amino]pyrrolidin-1-yl]-N-{8-fluoro-2-methylimidazo[1,2-a]pyridin-6-yl}-2-methylindazole-7-carboxamide (35 mg, 81%) as a solid. LCMS (ES, m/z): 473 [M+H]^{+ 1}H NMR (400 MHz, DMSO-d₆) δ 11.01 (s, 1H), 9.20 (d, J=1.6 Hz, 1H), 8.81 (s, 1H), 7.93 (d, J=8.2 Hz, 1H), 7.89 (d, J=3.1 Hz, 1H), 7.31 (d, J=12.4 Hz, 1H), 6.02 (d, J=8.3 Hz, 1H), 4.28 (s, 3H), 3.84-3.82 (m, 1H), 3.76-3.70 (m, 4H), 3.48 (d, J=10.0 Hz, 1H), 2.35 (s, 3H), 2.24-2.23 (m, 1H), 2.11-2.03 (m, 1H), 1.33-1.16 (m, 2H), 1.04 (d, J=12.4 Hz, 1H), 0.98 (d, J=4.3 Hz, 1H).

### Example 297: Synthesis of Compound 600

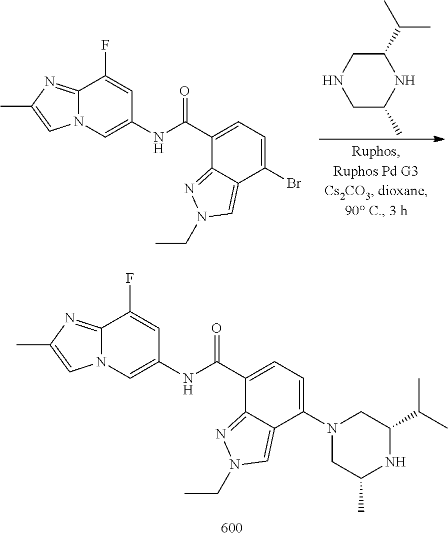

A solution of 4-bromo-2-ethyl-N-{8-fluoro-2-methylimidazo[1,2-a] pyridin-6-yl} indazole-7-carboxamide (120 mg, 0.288 mmol, 1 equiv) in 1,4-dioxane (1.5 mL) was added (2S,6R)-2-isopropyl-6-methylpiperazine (61.51 mg, 0.432 mmol, 1.5 equiv), Cs₂CO₃ (234.82 mg, 0.720 mmol, 2.5 equiv), RuPhos (26.91 mg, 0.058 mmol, 0.2 equiv) and RuPhos Palladacycle Gen.3 (24.11 mg, 0.029 mmol, 0.1 equiv) under nitrogen atmosphere. The reaction was stirred for 2 h at 90° C. The resulting mixture was concentrated under vacuum. The residue was purified by silica gel column chromatography, eluted with PE/EA (90%) to afford the crude product. The crude product was purified by Prep-HPLC (Condition 10, Gradient 16) to afford 2-ethyl-N-{8-fluoro-2-methylimidazo[1,2-a] pyridin-6-yl}-4-[(3S,5R)-3-isopropyl-5-methylpiperazin-1-yl] indazole-7-carboxamide (40 mg, 29%) as a solid. LCMS (ES, m/z): 478 [M+H]^{+ 1}H NMR (400 MHz, DMSO-d₆) δ 11.11 (s, 1H), 9.21 (d, J=1.6 Hz, 1H), 8.76 (s, 1H), 7.98 (d, J=8.1 Hz, 1H), 7.91 (d, J=3.0 Hz, 1H), 7.30 (dd, J=12.3, 1.7 Hz, 1H), 6.50 (d, J=8.2 Hz, 1H), 4.61 (q, J=7.2 Hz, 2H), 3.80 (dd, J=20.3, 10.2 Hz, 2H), 2.95-2.94 (m, 1H), 2.67-2.55 (m, 2H), 2.50-2.49 (m, 1H), 2.35 (s, 3H), 2.05 (s, 1H), 1.62 (t, J=7.3 Hz, 4H), 1.10 (d, J=6.2 Hz, 3H), 0.99 (dd, J=6.8, 1.5 Hz, 6H).

### Example 298: Synthesis of Compound 604

### Synthesis of Intermediate C493

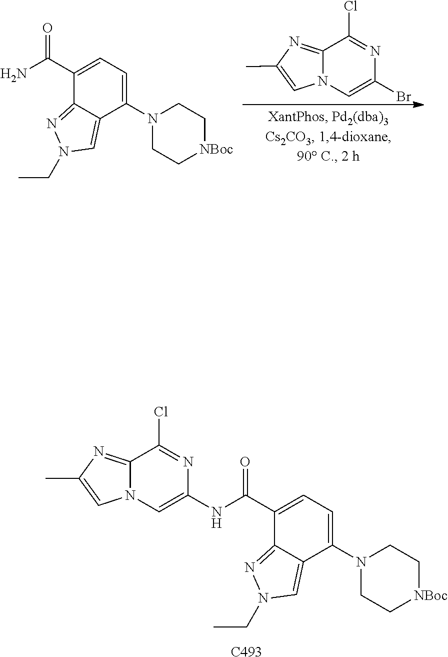

To a stirred solution of tert-butyl 4-(7-carbamoyl-2-ethylindazol-4-yl)piperazine-1-carboxylate (120 mg, 0.321 mmol, 1 equiv) and 6-bromo-8-chloro-2-methylimidazo[1,2-a]pyrazine (95 mg, 0.385 mmol, 1.2 equiv) in dioxane (2.4 mL) were added Cs₂CO₃ (314.1 mg, 0.963 mmol, 3 equiv) and XantPhos (37.2 mg, 0.064 mmol, 0.2 equiv) and Pd₂(dba)₃ (29.4 mg, 0.032 mmol, 0.1 equiv) at room temperature. The resulting mixture was stirred for 2 h at 90° C. under nitrogen atmosphere. The mixture was allowed to cool down to room temperature. The resulting mixture was diluted with water (3 mL). The resulting mixture was extracted with EtOAc (3×5 mL). The combined organic layers were washed with water (3×5 mL), dried over anhydrous Na₂SO₄. After filtration, the filtrate was concentrated under reduced pressure. The residue was purified by silica gel column chromatography, eluted with EA to afford tert-butyl 4-[7-({8-chloro-2-methylimidazo[1,2-a]pyrazin-6-yl}carbamoyl)-2-ethylindazol-4-yl]piperazine-1-carboxylate (150 mg, 87%) as a solid. LCMS (ES, m/z): 539 [M+H]⁺

### Synthesis of Compound 604

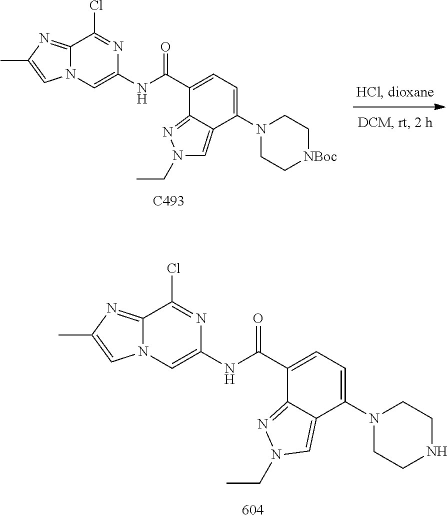

To a stirred mixture of tert-butyl 4-[7-({8-chloro-2-methylimidazo[1,2-a]pyrazin-6-yl}carbamoyl)-2-ethylindazol-4-yl]piperazine-1-carboxylate (100 mg, 0.186 mmol, 1 equiv) in DCM (1 mL) was added HCl(gas) in 1,4-dioxane (0.3 mL) dropwise at room temperature. The resulting mixture was stirred for 2 h at room temperature. The resulting mixture was concentrated under vacuum. The crude product was purified by Prep-HPLC (Condition 10, Gradient 2) to afford N-{8-chloro-2-methylimidazo[1,2-a]pyrazin-6-yl}-2-ethyl-4-(piperazin-1-yl) indazole-7-carboxamide (24 mg, 29%) as a solid. LCMS (ES, m/z): 439 [M+H]^{+ 1}H NMR (400 MHz, DMSO-d₆) δ 11.45 (s, 1H), 9.45 (s, 1H), 8.84 (s, 1H), 8.17 (s, 1H), 8.03 (d, J=8.1 Hz, 1H), 6.51 (d, J=8.2 Hz, 1H), 4.57 (q, J=7.3 Hz, 2H), 3.38 (t, J=4.9 Hz, 4H), 2.92 (dd, J=6.0, 3.5 Hz, 4H), 2.42 (s, 3H), 1.64 (t, J=7.3 Hz, 3H).

### Example 299: Synthesis of Compounds 605, 606, and 607

### Synthesis of Intermediate C494

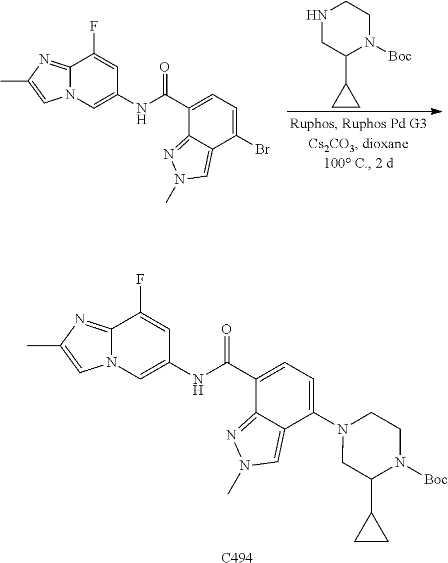 [SMILES: Cc1cn2cc(NC(=O)c3ccc(Br)c4cn(C)nc34)cc(F)c2n1 ; CC(C)(C)OC(=O)N1CCNCC1C1CC1 ; Cc1cn2cc(NC(=O)c3ccc(N4CCN(C(=O)OC(C)(C)C)C(C5CC5)C4)c4cn(C)nc34)cc(F)c2n1 ; "C494"]

To a solution of 4-bromo-N-{8-fluoro-2-methylimidazo[1,2-a]pyridin-6-yl}-2-methylindazole-7-carboxamide (110.0 mg, 0.273 mmol, 1.0 equiv) and tert-butyl 2-cyclopropylpiperazine-1-carboxylate (92.8 mg, 0.410 mmol, 1.5 equiv) in DMF (3 mL) were added Cs₂CO₃ (178.2 mg, 0.546 mmol, 2.0 equiv) and Ruphos (25.5 mg, 0.055 mmol, 0.2 equiv), RuPhos Palladacycle Gen.3 (22.8 mg, 0.027 mmol, 0.1 equiv). After stirring for 2 days at 100° C. under a nitrogen atmosphere, the resulting mixture was concentrated under reduced pressure. The residue was purified by silica gel column chromatography, eluted with PE/EA (1:2) to afford tert-butyl 2-cyclopropyl-4-[7-({8-fluoro-2-methylimidazo[1,2-a]pyridin-6-yl}carbamoyl)-2-methylindazol-4-yl]piperazine-1-carboxylate (150 mg, 85%) as a solid. LCMS (ES, m/z): 548 [M+H]⁺

### Synthesis of Compound 605

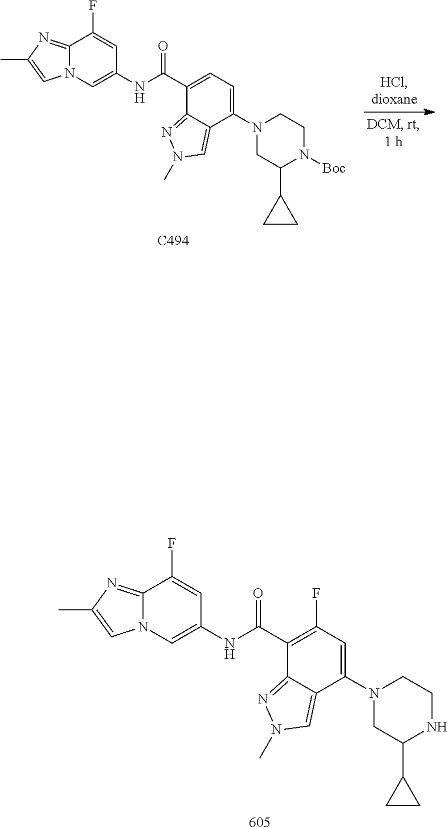 [SMILES: Cc1cn2cc(NC(=O)c3ccc(N4CCN(C(=O)OC(C)(C)C)C(C5CC5)C4)c4cn(C)nc34)cc(F)c2n1 ; "C494" ; Cc1cn2cc(NC(=O)c3c(F)cc(N4CCNC(C5CC5)C4)c4cn(C)nc34)cc(F)c2n1 ; "605"]

Into a 8 mL round-bottom flask were added tert-butyl 2-cyclopropyl-4-[7-({8-fluoro-2-methylimidazo[1,2-a]pyridin-6-yl}carbamoyl)-2-methylindazol-4-yl]piperazine-1-carboxylate (140.0 mg, 0.256 mmol, 1.0 equiv), DCM (2 mL) and HCl (gas) in 1,4-dioxane (1 mL, 4M) at room temperature. The resulting mixture was stirred for 1 h at room temperature. The resulting mixture was concentrated under reduced pressure. The crude product was purified by Prep-HPLC (Condition 13, Gradient 1) to afford 4-(3-cyclopropylpiperazin-1-yl)-N-{8-fluoro-2-methylimidazo[1,2-a]pyridin-6-yl}-2-methylindazole-7-carboxamide (18 mg, 16%) as a solid. LCMS (ES, m/z): 448 [M+H]^{+ 1}H NMR (400 MHz, DMSO-d₆) δ 11.07 (s, 1H), 9.21 (s, 1H), 8.74 (s, 1H), 7.98 (d, J=8.0 Hz, 1H), 7.91 (d, J=3.0 Hz, 1H), 7.35 (d, J=12.3 Hz, 1H), 6.50 (d, J=8.1 Hz, 1H), 4.31 (s, 3H), 3.78 (t, J=10.1 Hz, 2H), 3.17 (d, J=4.9 Hz, 1H), 3.02 (d, J=11.2 Hz, 1H), 2.94 (t, J=11.6 Hz, 1H), 2.81 (dt, J=22.0, 11.0 Hz, 2H), 2.35 (s, 3H), 2.08 (t, J=9.3 Hz, 1H), 0.84-0.76 (m, 1H), 0.43 (d, J=8.2 Hz, 2H), 0.36 (s, 1H), 0.33-0.26 (m, 1H).

### Synthesis of Compound 606

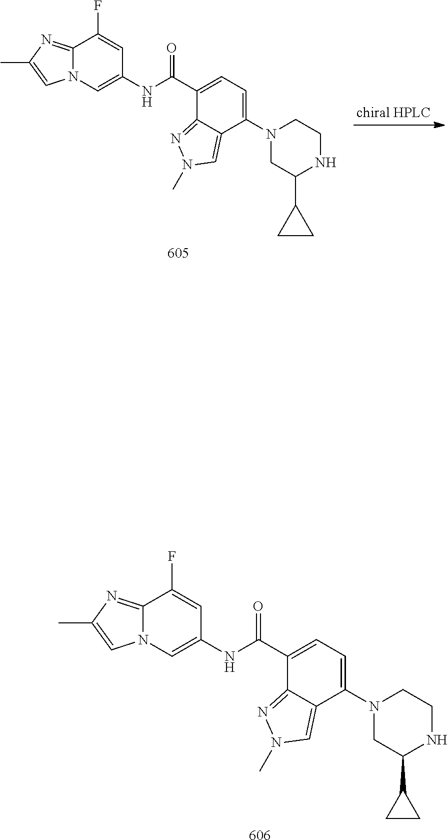 [SMILES: Cc1cn2cc(NC(=O)c3ccc(N4CCNC(C5CC5)C4)c4cn(C)nc34)cc(F)c2n1 ; "605" ; Cc1cn2cc(NC(=O)c3ccc(N4CCN[C@@H](C5CC5)C4)c4cn(C)nc34)cc(F)c2n1 ; "606"]

18 mg of 4-(3-cyclopropylpiperazin-1-yl)-N-{8-fluoro-2-methylimidazo[1,2-a]pyridin-6-yl}-2-methylindazole-7-carboxamide was purified by chiral-prep-HPLC (Condition 11, Gradient 1) to afford 4-[(3S)-3-cyclopropylpiperazin-1-yl]-N-{8-fluoro-2-methylimidazo[1,2-a]pyridin-6-yl}-2-methylindazole-7-carboxamide (4.6 mg, 26%) as a solid. LCMS (ES, m/z): 448 [M+H]^{+ 1}H NMR (400 MHz, DMSO-d₆) δ 11.07 (s, 1H), 9.21 (s, 1H), 8.74 (s, 1H), 7.98 (d, J=8.0 Hz, 1H), 7.91 (d, J=3.0 Hz, 1H), 7.35 (d, J=12.3 Hz, 1H), 6.50 (d, J=8.1 Hz, 1H), 4.31 (s, 3H), 3.78 (t, J=10.1 Hz, 2H), 3.17 (d, J=4.9 Hz, 1H), 3.02 (d, J=11.2 Hz, 1H), 2.94 (t, J=11.6 Hz, 1H), 2.81 (dt, J=22.0, 11.0 Hz, 2H), 2.35 (s, 3H), 2.08 (t, J=9.3 Hz, 1H), 0.84-0.76 (m, 1H), 0.43 (d, J=8.2 Hz, 2H), 0.36-0.35 (m, 1H), 0.33-0.26 (m, 1H).

### Synthesis of Compound 607

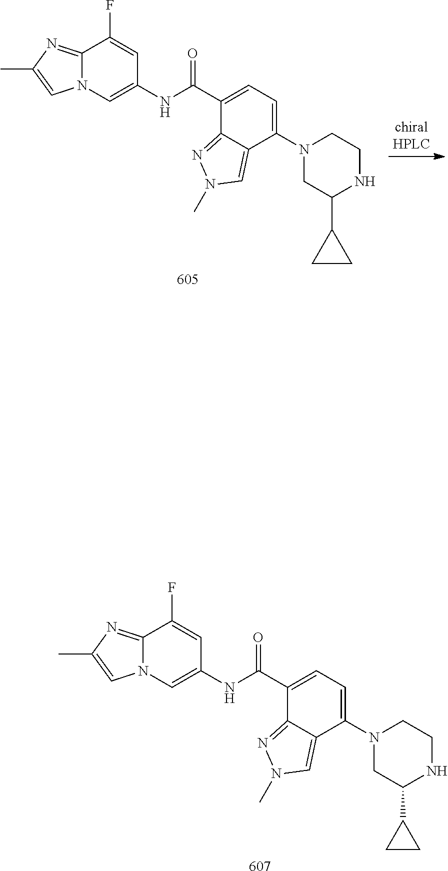 [SMILES: Cc1cn2cc(NC(=O)c3ccc(N4CCNC(C5CC5)C4)c4cn(C)nc34)cc(F)c2n1 ; "605" ; Cc1cn2cc(NC(=O)c3ccc(N4CCN[C@H](C5CC5)C4)c4cn(C)nc34)cc(F)c2n1 ; "607"]

18 mg of 4-(3-cyclopropylpiperazin-1-yl)-N-{8-fluoro-2-methylimidazo[1,2-a]pyridin-6-yl}-2-methylindazole-7-carboxamide was purified by chiral-prep-HPLC (Condition 11, Gradient 1) to afford 4-[(3R)-3-cyclopropylpiperazin-1-yl]-N-{8-fluoro-2-methylimidazo[1,2-a]pyridin-6-yl}-2-methylindazole-7-carboxamide (6 mg, 33%) as a solid. LCMS (ES, m/z): 448 [M+H]^{+ 1}H NMR (400 MHz, DMSO-d₆) δ 11.07 (s, 1H), 9.21 (s, 1H), 8.74 (s, 1H), 7.98 (d, J=8.0 Hz, 1H), 7.91 (d, J=3.0 Hz, 1H), 7.35 (d, J=12.3 Hz, 1H), 6.50 (d, J=8.1 Hz, 1H), 4.31 (s, 3H), 3.78 (t, J=10.1 Hz, 2H), 3.17 (d, J=4.9 Hz, 1H), 3.02 (d, J=11.2 Hz, 1H), 2.94 (t, J=11.6 Hz, 1H), 2.81 (dt, J=22.0, 11.0 Hz, 2H), 2.35 (s, 3H), 2.08 (t, J=9.3 Hz, 1H), 0.84-0.76 (m, 1H), 0.43 (d, J=8.2 Hz, 2H), 0.36-0.35 (m, 1H), 0.33-0.26 (m, 1H).

### Example 300: Synthesis of Compound 610

### Synthesis of Intermediate C495

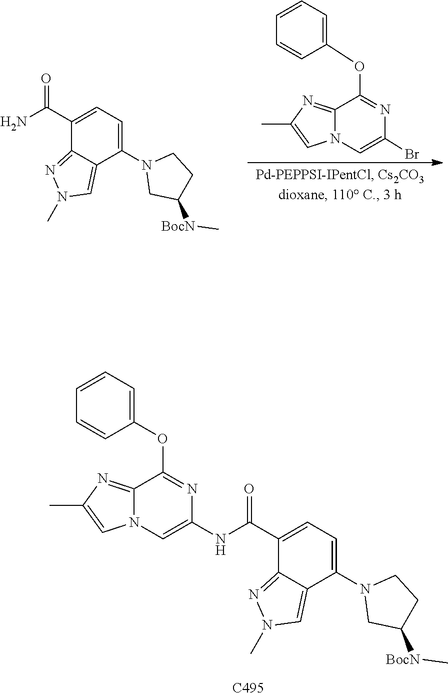

A solution of tert-butyl N-[(3R)-1-(7-carbamoyl-2-methylindazol-4-yl)pyrrolidin-3-yl]-N-methylcarbamate (100 mg, 0.268 mmol, 1 equiv), 6-bromo-2-methyl-8-phenoxyimidazo[1,2-a]pyrazine (122 mg, 0.402 mmol, 1.5 equiv), Cs₂CO₃ (261 mg, 0.804 mmol, 3 equiv) and Pd PEPPSI IPentCl (23 mg, 0.027 mmol, 0.1 equiv) in 1,4-dioxane was stirred for 3 h at 110° C. under nitrogen atmosphere. The mixture was allowed to cool down to room temperature. The resulting mixture was concentrated under reduced pressure. The residue was purified by silica gel column chromatography, eluted with PE/EA (0:1) to afford tert-butyl N-methyl-N-[(3R)-1-[2-methyl-7-({2-methyl-8-phenoxyimidazo[1,2-a]pyrazin-6-yl}carbamoyl) indazol-4-yl]pyrrolidin-3-yl]carbamate (96 mg, 60%) as a solid. LCMS (ES, m/z): 597 [M+H]⁺

### Synthesis of Compound 610

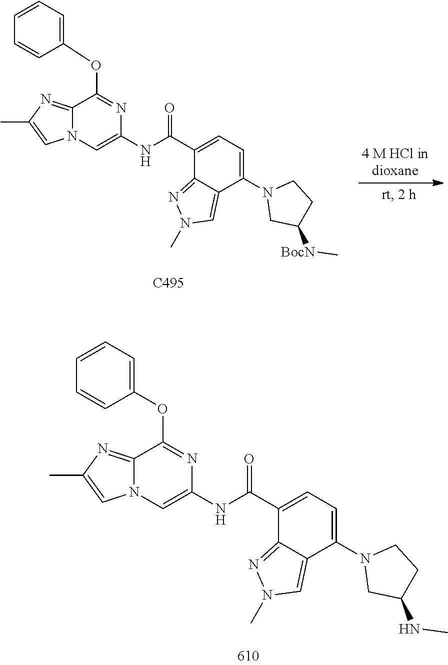

A solution of ert-butylN-methyl-N-[(3R)-1-[2-methyl-7-({2-methyl-8-phenoxyimidazo[1,2-a]pyrazin-6-yl}carbamoyl) indazol-4-yl]pyrrolidin-3-yl]carbamate (96 mg, 0.161 mmol, 1 equiv) in HCl(gas) in 1,4-dioxane (2 mL) was stirred for 2 h at room temperature. The resulting mixture was concentrated under vacuum. The residue was purified by reverse flash chromatography (Condition 3, Gradient 9) to afford 2-methyl-N-{2-methyl-8-phenoxyimidazo[1,2-a]pyrazin-6-yl}-4-[(3R)-3-(methylamino)pyrrolidin-1-yl]indazole-7-carboxamide (10 mg, 13%) as a solid. LCMS (ES, m/z): 497 [M+H]^{+ 1}H NMR (400 MHz, DMSO-d₆) δ 11.12 (s, 1H), 9.05 (s, 1H), 8.78 (s, 1H), 8.03 (s, 1H), 7.90 (d, J=8.3 Hz, 1H), 7.56 (t, J=7.8 Hz, 2H), 7.41 (d, J=7.9 Hz, 2H), 7.36 (t, J=7.3 Hz, 1H), 5.99 (d, J=8.4 Hz, 1H), 4.01 (s, 3H), 3.75 (s, 2H), 3.70 (s, 1H), 3.62 (s, 1H), 3.26 (s, 1H), 2.40 (s, 3H), 2.33 (s, 3H), 2.14 (dd, J=12.4, 6.2 Hz, 1H), 1.95-1.87 (m, 1H).

### Example 301: Synthesis of Compound 612

### Synthesis of Intermediate C496

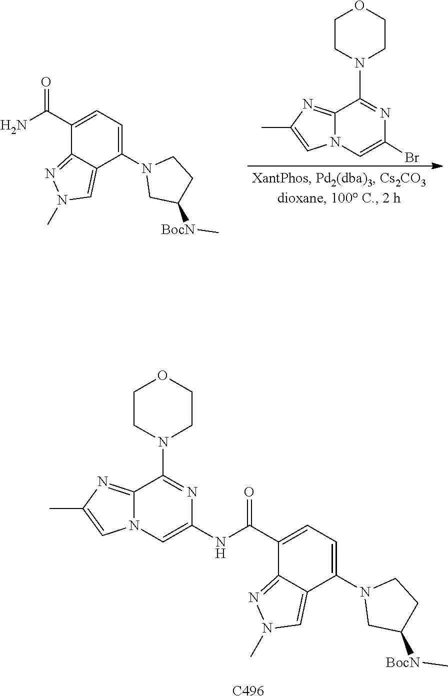

To a stirred solution of tert-butyl N-[(3R)-1-(7-carbamoyl-2-methylindazol-4-yl)pyrrolidin-3-yl]-N-methylcarbamate (100 mg, 0.268 mmol, 1 equiv), 4-{6-bromo-2-methylimidazo[1,2-a]pyrazin-8-yl}morpholine (90 mg, 0.322 mmol, 1.2 equiv) and Cs₂CO₃ (175 mg, 0.536 mmol, 2.0 equiv) in 1,4-dioxane (5 mL) were added XantPhos (31 mg, 0.0536 mmol, 0.2 equiv) and Pd₂(dba)₃ (25 mg, 0.027 mmol, 0.1 equiv) at room temperature under nitrogen atmosphere. The resulting mixture was stirred for 3 h at 90° C. The mixture was allowed to cool down to room temperature. The resulting mixture was concentrated under reduced pressure. The residue was purified by silica gel column chromatography, eluted with PE/EA (1:1) to afford tert-butyl N-methyl-N-[(3R)-1-(2-methyl-7-{[2-methyl-8-(morpholin-4-yl)imidazo[1,2-a]pyrazin-6-yl]carbamoyl}indazol-4-yl)pyrrolidin-3-yl]carbamate (96 mg, 61%) as a solid. LCMS (ES, m/z): 590 [M+H]⁺

### Synthesis of Compound 612

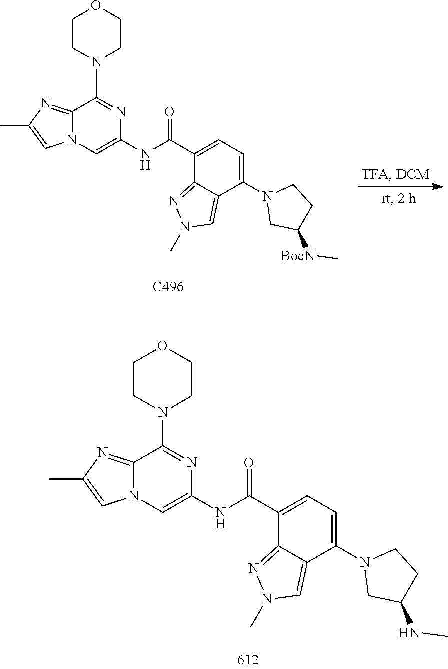

A solution of tert-butyl N-methyl-N-[(3R)-1-(2-methyl-7-{[2-methyl-8-(morpholin-4-yl)imidazo[1,2-a]pyrazin-6-yl]carbamoyl}indazol-4-yl)pyrrolidin-3-yl]carbamate (100 mg, 0.170 mmol, 1 equiv) and TFA (0.25 mL) in DCM (0.75 mL) was stirred for 2 h at room temperature. The resulting mixture was concentrated under reduced pressure. The residue was purified by reverse flash chromatography (Condition 3, Gradient 9) to afford 2-methyl-N-[2-methyl-8-(morpholin-4-yl)imidazo[1,2-a]pyrazin-6-yl]-4-[(3R)-3-(methylamino)pyrrolidin-1-yl]indazole-7-carboxamide (30 mg, 36%) as a solid. LCMS (ES, m/z): 490 [M+H]^{+ 1}H NMR (400 MHz, DMSO-d6) δ 11.08 (s, 1H), 8.81 (s, 1H), 8.72 (s, 1H), 7.92 (d, J=8.3 Hz, 1H), 7.78 (s, 1H), 6.00 (d, J=8.4 Hz, 1H), 4.23 (d, J=6.0 Hz, 7H), 3.78 (t, J=4.8 Hz, 6H), 3.74 (s, 1H), 3.64 (s, 1H), 3.42 (dd, J=10.3, 4.1 Hz, 1H), 2.33 (d, J=9.0 Hz, 6H), 2.15 (dq, J=13.2, 7.2 Hz, 1H), 2.08 (s, 1H), 1.92 (dt, J=11.5, 6.0 Hz, 1H)

### Example 302: Synthesis of Compound 614

### Synthesis of Intermediate C497

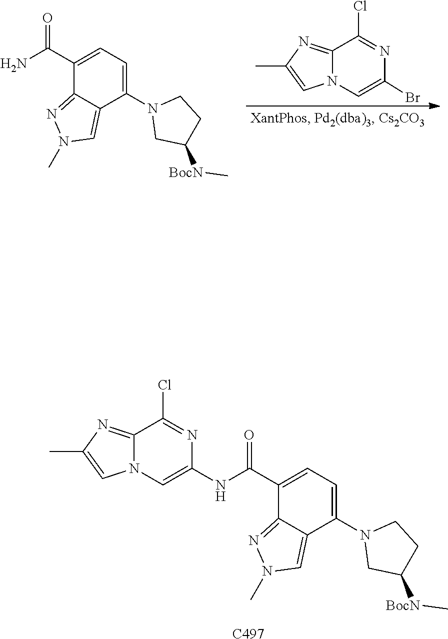

To a stirred solution of tert-butyl N-[(3R)-1-(7-carbamoyl-2-methylindazol-4-yl)pyrrolidin-3-yl]-N-methylcarbamate (65 mg, 0.174 mmol, 1 equiv), Cs₂CO₃ (170 mg, 0.522 mmol, 3 equiv) and 6-bromo-8-chloro-2-methylimidazo[1,2-a]pyrazine (64 mg, 0.261 mmol, 1.5 equiv) in 1,4-dioxane (3.0 mL) were added XantPhos (20 mg, 0.035 mmol, 0.2 equiv) and Pd₂(dba)₃ (15 mg, 0.017 mmol, 0.1 equiv) at room temperature under nitrogen atmosphere. The resulting mixture was stirred for 2 h at 90° C. under nitrogen atmosphere. The mixture was allowed to cool down to room temperature. The resulting mixture was concentrated under reduced pressure. The residue was purified by silica gel column chromatography, eluted with PE/EA (0:1) to afford tert-butyl N-[(3R)-1-[7-({8-chloro-2-methylimidazo[1,2-a]pyrazin-6-yl}carbamoyl)-2-methylindazol-4-yl]pyrrolidin-3-yl]-N-methylcarbamate (66 mg, 70%) as a solid. LCMS (ES, m/z): 539 [M+H]

### Synthesis of Compound 614

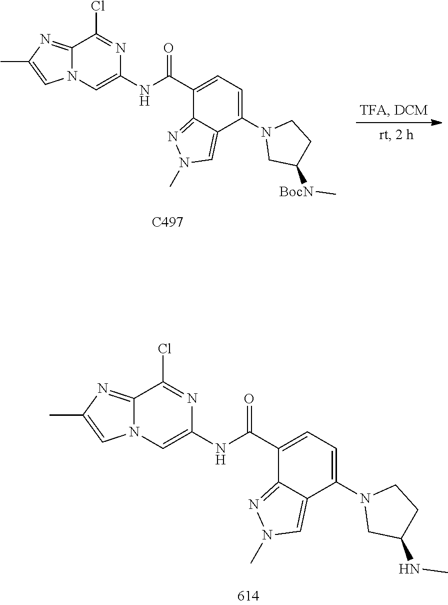

A solution of tert-butyl N-[(3R)-1-[7-({8-chloro-2-methylimidazo[1,2-a]pyrazin-6-yl}carbamoyl)-2-methylindazol-4-yl]pyrrolidin-3-yl]-N-methylcarbamate (66 mg, 0.122 mmol, 1 equiv) and TFA (0.25 mL) in DCM (0.75 mL) was stirred for 2 h at room temperature. The resulting mixture was concentrated under reduced pressure. The residue was purified by reverse flash chromatography (Condition 3, Gradient 3) to afford N-{8-chloro-2-methylimidazo[1,2-a]pyrazin-6-yl}-2-methyl-4-[(3R)-3-(methylamino)pyrrolidin-1-yl]indazole-7-carboxamide (10 mg, 19%) as a solid. LCMS (ES, m/z): 439 [M+H]^{+ 1}H NMR (400 MHz, DMSO-d6) δ 11.25 (s, 1H), 9.44 (s, 1H), 8.85 (s, 1H), 8.16 (s, 1H), 7.97 (d, J=8.4 Hz, 1H), 6.04 (d, J=8.4 Hz, 1H), 4.26 (s, 3H), 3.66 (s, 1H), 3.36-0.32 (m, 4H), 2.42 (s, 3H), 2.35 (s, 3H), 2.17 (dt, J=12.9, 6.5 Hz, −1H), 1.97-1.89 (m, 1H).

### Example 303: Synthesis of Compound 615, 616, and 617

### Synthesis of Compound 615

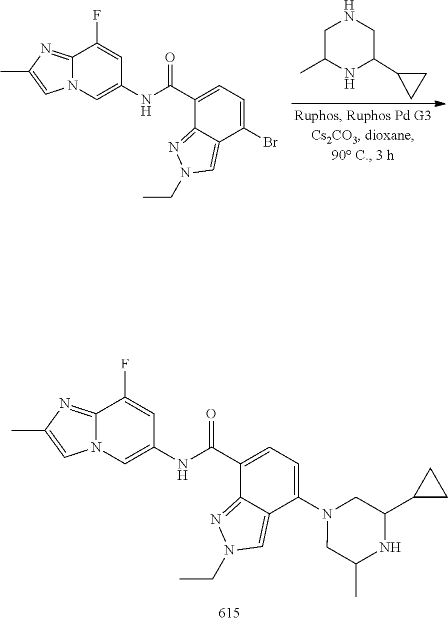

To a stirred mixture of 4-bromo-2-ethyl-N-{8-fluoro-2-methylimidazo[1,2-a]pyridin-6-yl}indazole-7-carboxamide (150 mg, 0.360 mmol, 1 equiv) and 2-cyclopropyl-6-methylpiperazine (65.7 mg, 0.468 mmol, 1.3 equiv) in dioxane (3 mL) were added Cs₂CO₃ (352.2 mg, 1.080 mmol, 3 equiv) and Ruphos (33.6 mg, 0.072 mmol, 0.2 equiv) and RuPhos Palladacycle Gen.3 (30.1 mg, 0.036 mmol, 0.1 equiv) at room temperature under nitrogen atmosphere. The resulting mixture was stirred for 3 h at 90° C. under nitrogen atmosphere. The resulting mixture was diluted with water (10 mL). The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by silica gel column chromatography, eluted with CH₂Cl₂/MeOH (10:1) to afford 4-(3-cyclopropyl-5-methylpiperazin-1-yl)-2-ethyl-N-{8-fluoro-2-methylimidazo[1,2-a]pyridin-6-yl}indazole-7-carboxamide (80 mg, 47%) as a solid. LCMS (ES, m/z): 476 [M+H]⁺

### Synthesis of Compound 616

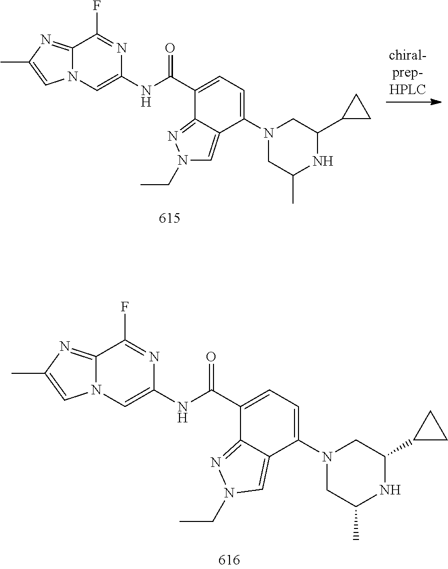

80 mg of 4-(3-cyclopropyl-5-methylpiperazin-1-yl)-2-ethyl-N-(8-fluoro-2-methylimidazo[1,2-a]pyridin-6-yl)-2H-indazole-7-carboxamide was purified by chiral-prep-HPLC (Condition 12, Gradient 1) to afford 4-((3S,5R)-3-cyclopropyl-5-methylpiperazin-1-yl)-2-ethyl-N-(8-fluoro-2-methylimidazo[1,2-a]pyridin-6-yl)-2H-indazole-7-carboxamide (8.9 mg, 11.12%) as a yellow solid. LCMS (ES, m/z): 476 [M+H]^{+ 1}H NMR (400 MHz, DMSO-d₆) δ 11.11 (s, 1H), 9.21 (d, J=1.7 Hz, 1H), 8.78 (s, 1H), 7.98 (d, J=8.1 Hz, 1H), 7.91 (d, J=3.1 Hz, 1H), 7.30 (dd, J=12.3, 1.7 Hz, 1H), 6.50 (d, J=8.2 Hz, 1H), 4.61 (q, J=7.3 Hz, 2H), 3.84-3.76 (m, 2H), 2.92 (d, J=8.7 Hz, 1H), 2.72 (d, J=10.9 Hz, 1H), 2.50-2.49 (m, 1H), 2.35 (s, 3H), 2.18-2.09 (m, 1H), 1.62 (t, J=7.2 Hz, 3H), 1.09 (d, J=6.2 Hz, 3H), 0.77 (qt, J=8.1, 4.8 Hz, 1H), 0.48-0.27 (m, 4H).

### Synthesis of Compound 617

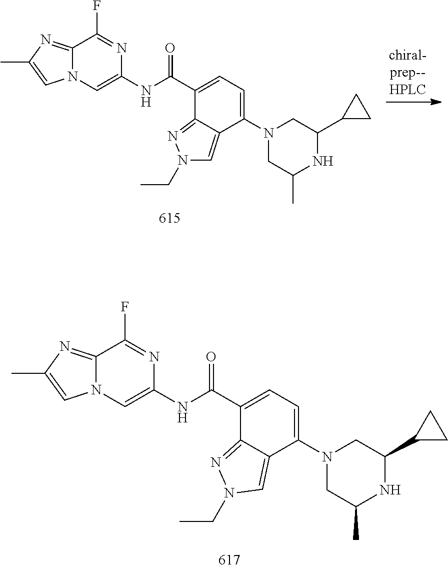

80 mg of 4-(3-cyclopropyl-5-methylpiperazin-1-yl)-2-ethyl-N-(8-fluoro-2-methylimidazo[1,2-a]pyridin-6-yl)-2H-indazole-7-carboxamide was purified by chiral-prep-HPLC (Condition 12, Gradient 1) to afford 4-((3R,5S)-3-cyclopropyl-5-methylpiperazin-1-yl)-2-ethyl-N-(8-fluoro-2-methylimidazo[1,2-a]pyridin-6-yl)-2H-indazole-7-carboxamide (16.5 mg, 21%) as a solid. LCMS (ES, m/z): 476 [M+H]^{+ 1}H NMR (400 MHz, DMSO-d₆) δ 11.11 (s, 1H), 9.21 (d, J=1.7 Hz, 1H), 8.78 (s, 1H), 7.98 (d, J=8.1 Hz, 1H), 7.91 (d, J=3.1 Hz, 1H), 7.30 (dd, J=12.3, 1.7 Hz, 1H), 6.50 (d, J=8.2 Hz, 1H), 4.61 (q, J=7.3 Hz, 2H), 3.84-3.76 (m, 2H), 2.92 (d, J=8.7 Hz, 1H), 2.72 (d, J=10.9 Hz, 1H), 2.50-2.49 (m, 1H), 2.35 (s, 3H), 2.18-2.09 (m, 1H), 1.62 (t, J=7.2 Hz, 3H), 1.09 (d, J=6.2 Hz, 3H), 0.77 (qt, J=8.1, 4.8 Hz, 1H), 0.48-0.27 (m, 4H).

### Example 304: Synthesis of Compound 619

### Synthesis of Intermediate C498

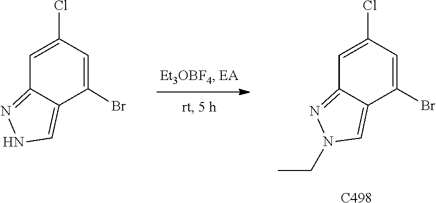

To a solution of 4-bromo-6-chloro-2H-indazole (2.0 g, 8.651 mmol, 1 equiv) in EA (30 mL) was added triethyloxonium tetrafluoroborate (1.98 g, 10.389 mmol, 1.2 equiv) in portions at 0 degrees C. The resulting mixture was stirred for 5 h at room temperature. The reaction mixture was quenched with saturated aqueous NaHCO₃ (50 mL). The resulting mixture was extracted with EA (3×20 mL). The combined organics were washed with brine (2×10 mL), dried over anhydrous Na₂SO₄. After filtration, the filtrate was concentrated under reduced pressure to afford 4-bromo-6-chloro-2-ethyl-2H-indazole (1.8 g, 80%) as a solid. LCMS (ES, m/z): 259 [M+H]⁺

### Synthesis of Intermediate C499

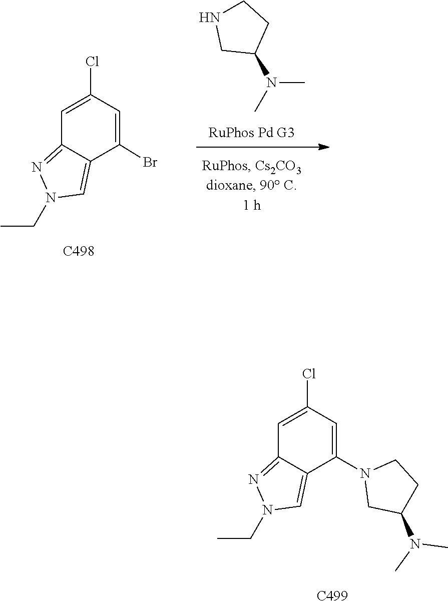

To a solution of 4-bromo-6-chloro-2-ethylindazole (600 mg, 2.312 mmol, 1.0 equiv) and (3R)—N,N-dimethylpyrrolidin-3-amine (316 mg, 2.774 mmol, 1.2 equiv) in dioxane (6.0 mL) were added Cs₂CO₃ (1.5 g, 4.624 mmol, 2.0 equiv), RuPhos Palladacycle Gen.3 (193 mg, 0.231 mmol, 0.1 equiv) and RuPhos (107 mg, 0.231 mmol, 0.1 equiv) at room temperature. After stirring for 1 h at 90° C. under a nitrogen atmosphere, the resulting mixture was concentrated under reduced pressure. The residue was purified by silica gel column chromatography, eluted with CH₂Cl₂/MeOH (20:1) to afford (3R)-1-(6-chloro-2-ethylindazol-4-yl)-N,N-dimethylpyrrolidin-3-amine (600 mg, 89%) as a solid. LCMS (ES, m/z): 293 [M+H]⁺

### Synthesis of Intermediate C500

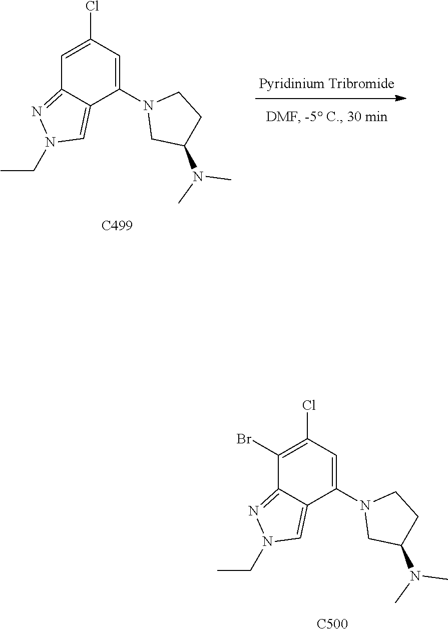 [SMILES: CCn1cc2c(N3CC[C@@H](N(C)C)C3)cc(Cl)cc2n1 ; "C499" ; CCn1cc2c(N3CC[C@@H](N(C)C)C3)cc(Cl)c(Br)c2n1 ; "C500"]

To a stirred solution of (3R)-1-(6-chloro-2-ethylindazol-4-yl)-N,N-dimethylpyrrolidin-3-amine (600 mg, 2.049 mmol, 1.0 equiv) in DMF (12 mL) was added pyridinium tribromide (590 mg, 1.844 mmol, 0.9 equiv) in portions at −10° C. The resulting mixture was stirred for 1 h at −10° C. The reaction was quenched with water (2 mL) at 0° C. The mixture was purified by reverse flash chromatography (Condition 3, Gradient 1) to afford (3R)-1-(7-bromo-6-chloro-2-ethylindazol-4-yl)-N,N-dimethylpyrrolidin-3-amine (350 mg, 46%) as a solid. LCMS (ES, m/z): 371 [M+H]⁺

### Synthesis of Intermediate C501

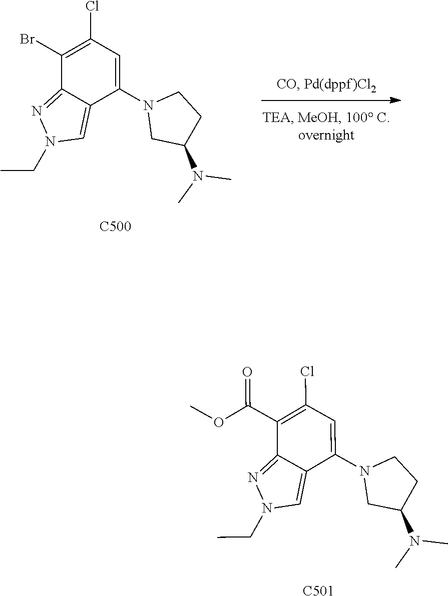

To a solution of (3R)-1-(7-bromo-6-chloro-2-ethylindazol-4-yl)-N,N-dimethylpyrrolidin-3-amine (350 mg, 0.942 mmol, 1.0 equiv) in MeOH (4 mL) were added Pd(dppf)Cl₂ (68 mg, 0.094 mmol, 0.1 equiv) and TEA (476 mg, 4.710 mmol, 5.0 equiv) in a pressure tank. The mixture was purged with nitrogen for 5 min and then was pressurized to 20 atm with carbon monoxide at 100° C. for overnight. The reaction mixture was cooled to room temperature and filtered to remove insoluble solids. The filtrate was concentrated under reduced pressure. The residue was purified by reverse flash chromatography (Condition 3, Gradient 1) to afford methyl 6-chloro-4-[(3R)-3-(dimethylamino)pyrrolidin-1-yl]-2-ethylindazole-7-carboxylate (310 mg, 94%) as a solid. LCMS (ES, m/z): 351 [M+H]⁺

### Synthesis of Intermediate C502

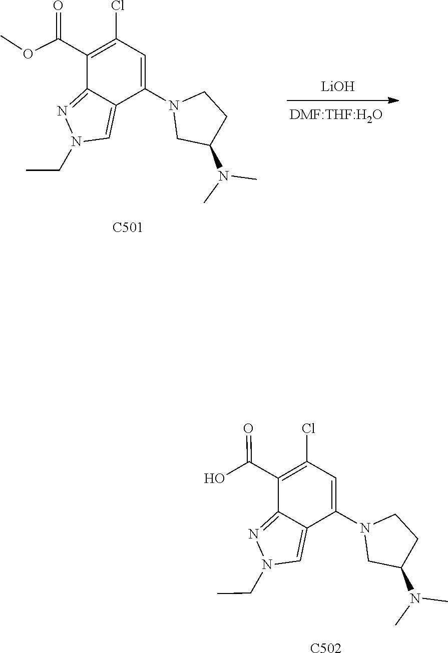

A mixture of methyl 6-chloro-4-[(3R)-3-(dimethylamino)pyrrolidin-1-yl]-2-ethylindazole-7-carboxylate (310 mg, 0.884 mmol, 1.0 equiv) and LiOH (211 mg, 8.840 mmol, 10.0 equiv) in THF (1 mL), MeOH (1 mL) and H₂O (1 mL) was stirred for 2 h at 50° C. The resulting mixture was concentrated under reduced pressure. The residue was purified by reverse flash chromatography (Condition 5, Gradient 2) to afford 6-chloro-4-[(3R)-3-(dimethylamino)pyrrolidin-1-yl]-2-ethylindazole-7-carboxylic acid (150 mg, 50%) as a solid. LCMS (ES, m/z): 337 [M+H]⁺

### Synthesis of Compound 619

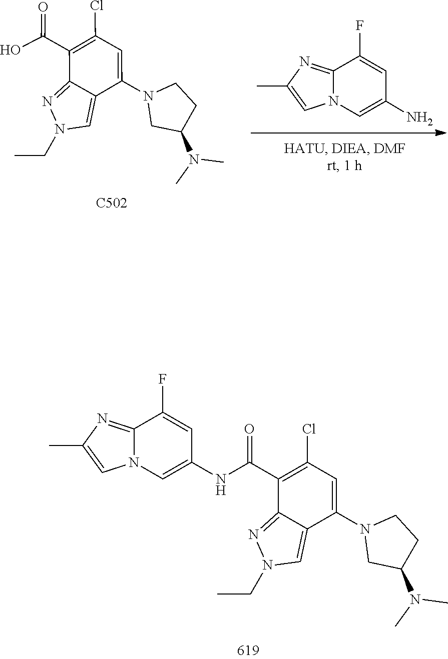

To a solution of 6-chloro-4-[(3R)-3-(dimethylamino)pyrrolidin-1-yl]-2-ethylindazole-7-carboxylic acid (150 mg, 0.445 mmol, 1.0 equiv) and DIEA (201 mg, 1.558 mmol, 3.5 equiv) in DMF (4 mL) were added HATU (338 mg, 0.890 mmol, 2.0 equiv) and 8-fluoro-2-methylimidazo[1,2-a]pyridin-6-amine (110 mg, 0.667 mmol, 1.5 equiv) at room temperature. The resulting mixture was stirred for 1 h at room temperature. The resulting mixture was quenched with water (10 mL). The resulting solids were collected by filtration and purified by reverse flash chromatography (Condition 3, Gradient 1) to afford 6-chloro-4-[(3R)-3-(dimethylamino)pyrrolidin-1-yl]-2-ethyl-N-{8-fluoro-2-methylimidazo[1,2-a]pyridin-6-yl}indazole-7-carboxamide (62 mg, 29%) as a solid. LCMS (ES, m/z): 484 [M+H]^{+ 1}H NMR (400 MHz, DMSO-d₆) δ 10.75 (s, 1H), 9.23 (s, 1H), 8.78 (s, 1H), 7.92 (d, J=2.9 Hz, 1H), 7.16 (d, J=12.5 Hz, 1H), 5.89 (s, 1H), 4.45 (q, J=7.3 Hz, 2H), 3.82-3.68 (m, 2H), 3.61 (d, J=9.5 Hz, 1H), 3.41 (d, J=9.1 Hz, 2H), 2.87 (d, J=9.3 Hz, 1H), 2.35 (s, 3H), 2.26 (s, 6H), 1.93-1.84 (m, 1H), 1.52 (t, J=7.3 Hz, 3H).

### Example 305: Synthesis of Compound 621

### Synthesis of Intermediate C503

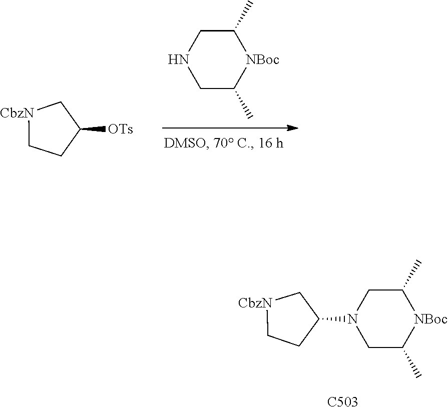 [SMILES: Cc1ccc(S(=O)(=O)O[C@H]2CCN(C(=O)OCc3ccccc3)C2)cc1 ; C[C@@H]1CNC[C@H](C)N1C(=O)OC(C)(C)C ; C[C@@H]1CN([C@@H]2CCN(C(=O)OCc3ccccc3)C2)C[C@H](C)N1C(=O)OC(C)(C)C ; "C503"]

A solution of benzyl (3S)-3-[(4-methylbenzenesulfonyl)oxy]pyrrolidine-1-carboxylate (2 g, 5.328 mmol, 1.0 equiv) in DMSO (5 mL) was treated with tert-butyl (2R,6S)-2,6-dimethylpiperazine-1-carboxylate (3.42 g, 15.67 mmol, 3.0 equiv) at room temperature. The resulting mixture was stirred for 16 h at 70° C. under nitrogen atmosphere. The mixture was allowed to cool down to room temperature. The resulting mixture was diluted with water. The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (1:2) to afford tert-butyl (2R,6S)-4-[(3R)-1-[(benzyloxy)carbonyl]pyrrolidin-3-yl]-2,6-dimethylpiperazine-1-carboxylate (710 mg, 52%) as an oil. LCMS (ES, m/z): 418 [M+H]⁺

### Synthesis of Intermediate C504

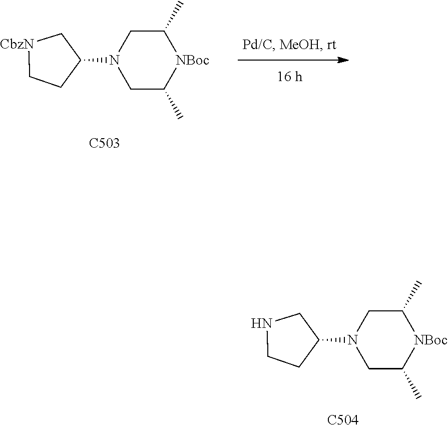 [SMILES: C[C@@H]1CN([C@@H]2CCN(C(=O)OCc3ccccc3)C2)C[C@H](C)N1C(=O)OC(C)(C)C ; "C503" ; C[C@@H]1CN([C@@H]2CCNC2)C[C@H](C)N1C(=O)OC(C)(C)C ; "C504"]

A solution of tert-butyl (2R,6S)-4-[(3R)-1-[(benzyloxy)carbonyl]pyrrolidin-3-yl]-2,6-dimethylpiperazine-1-carboxylate (710 mg, 0.431 mmol, 1 equiv) in MeOH (10 mL) was treated with Pd/C (71 mg) at room temperature. The resulting mixture was stirred for 16 h at room temperature under H₂ atmosphere. The resulting mixture was filtered and the filter cake was washed with MeOH (20 mL). The filtrate was concentrated under reduced pressure to afford tert-butyl (2R,6S)-2,6-dimethyl-4-[(3R)-pyrrolidin-3-yl]piperazine-1-carboxylate (322 mg, 83%) as an oil. LCMS (ES, m/z): 284 [M+H]⁺

### Synthesis of Intermediate C505

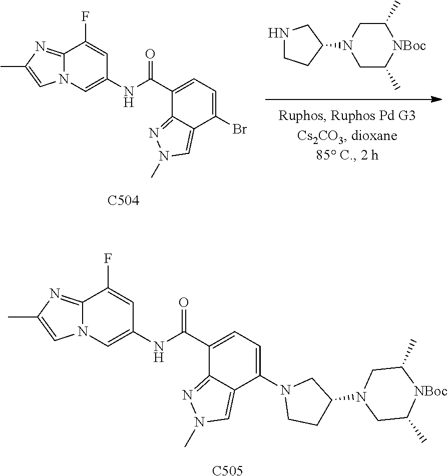

To a stirred solution of 4-bromo-N-{8-fluoro-2-methylimidazo[1,2-a]pyridin-6-yl}-2-methylindazole-7-carboxamide (300 mg, 0.746 mmol, 1.0 equiv) and tert-butyl (2R,6S)-2,6-dimethyl-4-[(3R)-pyrrolidin-3-yl]piperazine-1-carboxylate (253.6 mg, 0.895 mmol, 1.2 equiv) in dioxane (5 mL) were added Cs₂CO₃ (729.0 mg, 2.238 mmol, 3.0 equiv), Ruphos (69.6 mg, 0.149 mmol, 0.2 equiv) and RuPhos Palladacycle Gen.3 (62.3 mg, 0.075 mmol, 0.1 equiv) at room temperature under nitrogen atmosphere. The resulting mixture was stirred for 2 h at 85° C. under nitrogen atmosphere. The resulting mixture was concentrated under reduced pressure. The residue was purified by silica gel column chromatography, eluted with CH₂Cl₂/MeOH (10:1) to afford tert-butyl (2R,6S)-4-[(3R)-1-[7-({8-fluoro-2-methylimidazo[1,2-a]pyridin-6-yl}carbamoyl)-2-methylindazol-4-yl]pyrrolidin-3-yl]-2,6-dimethylpiperazine-1-carboxylate (238 mg, 53%) as a solid. LCMS (ES, m/z): 605 [M+H]⁺

### Synthesis of Compound 621

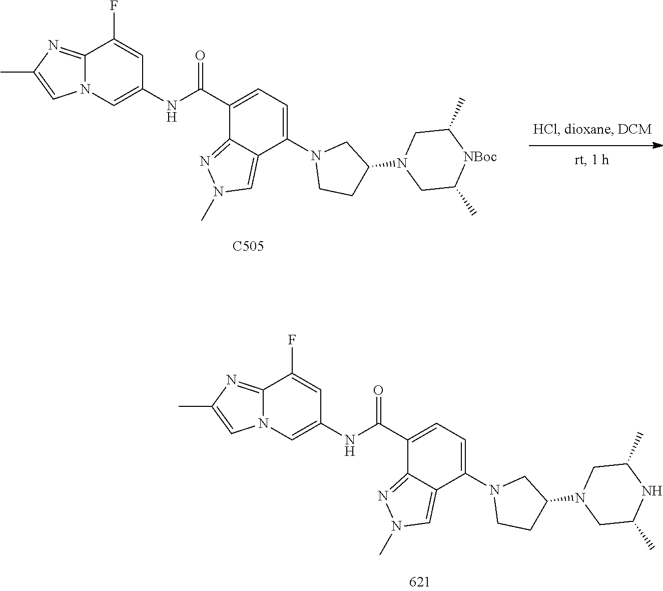

A solution of tert-butyl (2R,6S)-4-[(3R)-1-[7-({8-fluoro-2-methylimidazo[1,2-a]pyridin-6-yl}carbamoyl)-2-methylindazol-4-yl]pyrrolidin-3-yl]-2,6-dimethylpiperazine-1-carboxylate (110 mg, 0.182 mmol, 1 equiv) in DCM (1 mL) was treated with HCl (gas) in 1,4-dioxane (0.2 mL) at room temperature. The resulting mixture was stirred for 1 h at room temperature. The resulting mixture was concentrated under reduced pressure. The crude product was purified by Prep-HPLC (Condition 10, Gradient 11) to afford 4-[(3R)-3-[(3R,5S)-3,5-dimethylpiperazin-1-yl]pyrrolidin-1-yl]-N-{8-fluoro-2-methylimidazo[1,2-a]pyridin-6-yl}-2-methylindazole-7-carboxamide (34 mg, 37%) as a solid. LCMS (ES, m/z): 505 [M+H]^{+ 1}H NMR (400 MHz, DMSO-d₆) δ 11.02 (s, 1H), 9.19 (d, J=1.7 Hz, 1H), 8.86 (s, 1H), 7.92 (d, J=8.3 Hz, 1H), 7.89 (d, J=3.1 Hz, 1H), 7.30 (dd, J=12.5, 1.7 Hz, 1H), 6.05 (d, J=8.4 Hz, 1H), 4.27 (s, 3H), 3.86 (t, J=8.5 Hz, 1H), 3.74 (t, J=9.4 Hz, 1H), 3.61 (d, J=8.4 Hz, 1H), 3.46 (t, J=9.0 Hz, 1H), 2.94 (p, J=7.6 Hz, 1H), 2.89-2.70 (m, 4H), 2.35 (s, 3H), 2.27 (dd, J=12.2, 6.2 Hz, 1H), 1.88 (d, J=12.1 Hz, 2H), 1.60 (q, J=10.2 Hz, 2H), 0.96 (t, J=6.4 Hz, 6H).

### Example 306: Synthesis of Compound 622

### Synthesis of Intermediate C506

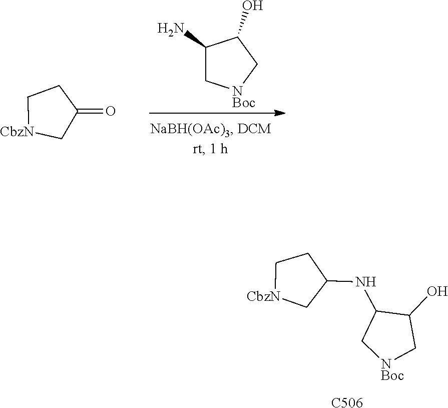 [SMILES: O=C1CCN(C(=O)OCc2ccccc2)C1 ; CC(C)(C)OC(=O)N1C[C@@H](N)[C@H](O)C1 ; CC(C)(C)OC(=O)N1CC(O)C(NC2CCN(C(=O)OCc3ccccc3)C2)C1 ; "C506"]

To a stirred mixture of benzyl 3-oxopyrrolidine-1-carboxylate (550 mg, 2.509 mmol, 1.0 equiv) and tert-butyl (3R,4R)-3-amino-4-hydroxypyrrolidine-1-carboxylate (608.8 mg, 3.011 mmol, 1.2 equiv) in DCE (5.5 mL) was added NaBH(AcO)₃ (797.5 mg, 3.763 mmol, 1.5 equiv) at room temperature under nitrogen atmosphere. The resulting mixture was stirred for 1 h at room temperature. The reaction was quenched with water at room temperature. The resulting mixture was concentrated under reduced pressure. The residue was purified by silica gel column chromatography, eluted with PE/EA (80%) to afford tert-butyl 3-({1-[(benzyloxy)carbonyl]pyrrolidin-3-yl}amino)-4-hydroxypyrrolidine-1-carboxylate (802 mg, 79%) as a solid. LCMS (ES, m/z): 406 [M+H]⁺

### Synthesis of Intermediate C507

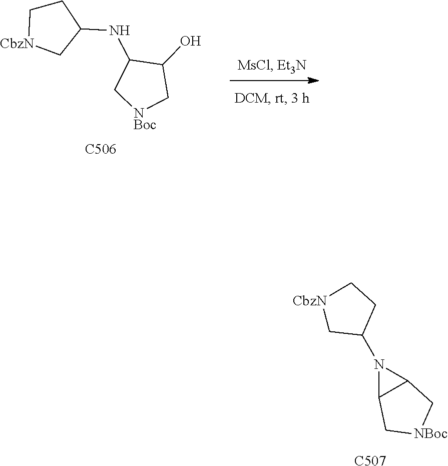

To a stirred mixture of tert-butyl 3-({1-[(benzyloxy)carbonyl]pyrrolidin-3-yl}amino)-4-hydroxypyrrolidine-1-carboxylate (800 mg, 1.973 mmol, 1.0 equiv) and TEA (399.2 mg, 3.946 mmol, 2.0 equiv) in DCM (8 mL) was added MsCl (271.1 mg, 2.368 mmol, 1.2 equiv) dropwise at room temperature under nitrogen atmosphere. The resulting mixture was stirred for 3 h at room temperature under nitrogen atmosphere. The reaction was quenched with Water at room temperature. The resulting mixture was extracted with CH₂Cl₂ (3×10 mL). The combined organic layers were washed with brine (1×10 mL), dried over anhydrous Na₂SO₄. After filtration, the filtrate was concentrated under reduced pressure to afford tert-butyl 6-{1-[(benzyloxy)carbonyl]pyrrolidin-3-yl}-3,6-diazabicyclo[3.1.0]hexane-3-carboxylate (623 mg, 82%) as a solid. LCMS (ES, m/z): 388 [M+H]⁺

### Synthesis of Intermediate C508

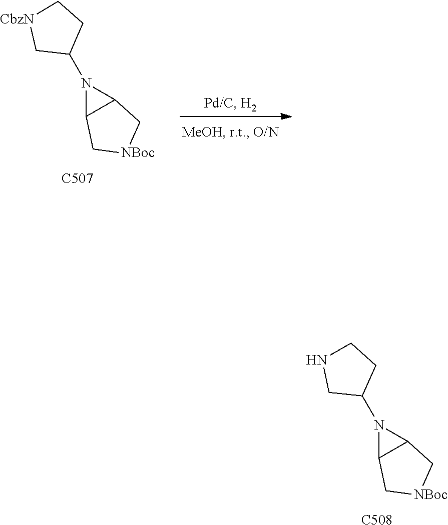

A solution of tert-butyl 6-{1-[(benzyloxy)carbonyl]pyrrolidin-3-yl}-3,6-diazabicyclo[3.1.0]hexane-3-carboxylate (623 mg, 1.608 mmol, 1 equiv) in MeOH (20 mL) was treated with Pd/C (62 mg) at room temperature. The resulting mixture was stirred for 16 h at room temperature under H₂ atmosphere. The resulting mixture was filtered and the filter cake was washed with MeOH (10 mL). The filtrate was concentrated under reduced pressure to afford tert-butyl 6-(pyrrolidin-3-yl)-3,6-diazabicyclo[3.1.0]hexane-3-carboxylate (280 mg, 69%) as an oil. LCMS (ES, m/z): 254 [M+H]⁺

### Synthesis of Intermediate C509

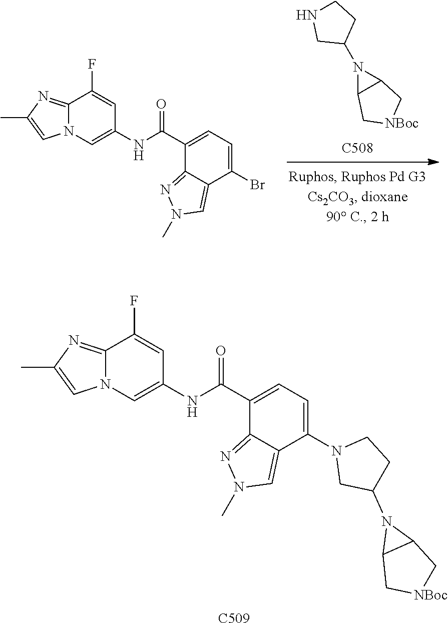

To a stirred mixture of 4-bromo-N-{8-fluoro-2-methylimidazo[1,2-a]pyridin-6-yl}-2-methylindazole-7-carboxamide (220.0 mg, 0.547 mmol, 1.0 equiv) and tert-butyl 6-(pyrrolidin-3-yl)-3,6-diazabicyclo[3.1.0]hexane-3-carboxylate (207.8 mg, 0.821 mmol, 1.5 equiv) in 1,4-dioxane (4 mL) were added Ruphos (51.0 mg, 0.109 mmol, 0.2 equiv), Cs₂CO₃ (534.6 mg, 1.641 mmol, 3.0 equiv) and RuPhos Palladacycle Gen.3 (56.2 mg, 0.11 mmol, 0.1 equiv) at room temperature under nitrogen atmosphere. The resulting mixture was stirred for 2 h at 90° C. under nitrogen atmosphere. The mixture was allowed to cool down to room temperature. The resulting mixture was diluted with water. The resulting mixture was extracted with EtOAc (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl 6-{1-[7-({8-fluoro-2-methylimidazo[1,2-a]pyridin-6-yl}carbamoyl)-2-methylindazol-4-yl]pyrrolidin-3-yl}-3,6-diazabicyclo[3.1.0]hexane-3-carboxylate (260 mg, 83%) as a solid. LCMS (ES, m/z): 575 [M+H]⁺

### Synthesis of Compound 622

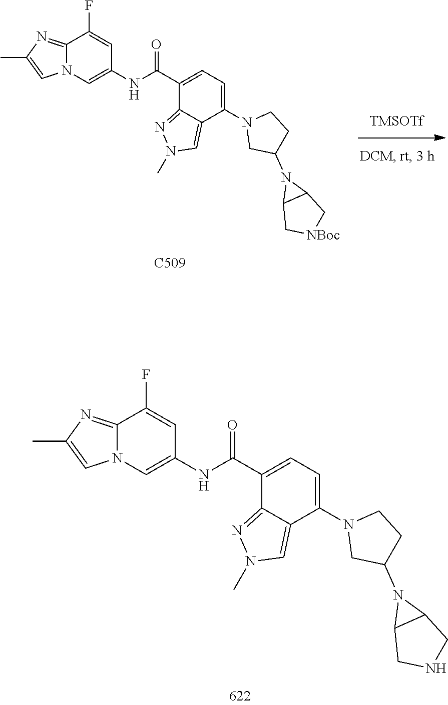 [SMILES: "TMSOTf" ; Cc1cn2cc(NC(=O)c3ccc(N4CCC(N5C6CN(C(=O)OC(C)(C)C)CC65)C4)c4cn(C)nc34)cc(F)c2n1 ; "C509" ; Cc1cn2cc(NC(=O)c3ccc(N4CCC(N5C6CNCC65)C4)c4cn(C)nc34)cc(F)c2n1]

To a stirred mixture of tert-butyl 6-{1-[7-({8-fluoro-2-methylimidazo[1,2-a]pyridin-6-yl}carbamoyl)-2-methylindazol-4-yl]pyrrolidin-3-yl}-3,6-diazabicyclo[3.1.0]hexane-3-carboxylate (60 mg, 0.104 mmol, 1 equiv) and DIEA (67.4 mg, 0.520 mmol, 5 equiv) in DCM (1 mL) was added TMSOTf (116.0 mg, 0.520 mmol, 5 equiv) at room temperature. The resulting mixture was stirred for 3 h at room temperature. The resulting mixture was concentrated under reduced pressure. The crude product was purified by Prep-HPLC (Condition 10, Gradient 3) to afford 4-(3-{3,6-diazabicyclo[3.1.0]hexan-6-yl}pyrrolidin-1-yl)-N-{8-fluoro-2-methylimidazo[1,2-a]pyridin-6-yl}-2-methylindazole-7-carboxamide (15 mg, 30%) as a solid. LCMS (ES, m/z): 475 [M+H]^{+ 1}H NMR (400 MHz, DMSO-d₆) δ 11.01 (s, 1H), 9.20 (d, J=1.7 Hz, 1H), 8.84 (s, 1H), 7.93 (d, J=8.3 Hz, 1H), 7.89 (d, J=3.1 Hz, 1H), 7.31 (dd, J=12.4, 1.7 Hz, 1H), 6.02 (d, J=8.3 Hz, 1H), 4.27 (s, 3H), 3.81 (d, J=9.5 Hz, 2H), 3.68 (s, 1H), 3.52 (d, J=10.4 Hz, 1H), 2.82 (t, J=11.8 Hz, 2H), 2.56-2.54 (m, 2H), 2.41 (dd, J=14.8, 12.3 Hz, 1H), 2.34-2.32 (m, 5H), 2.14 (tt, J=13.8, 7.0 Hz, 1H), 2.08-1.96 (m, 1H).

### Example 307: Synthesis of Compound 624

### Synthesis of Intermediate C510

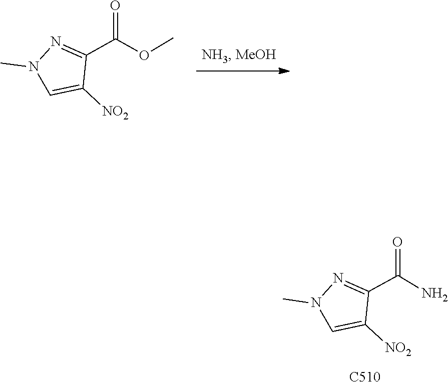

A solution of methyl 1-methyl-4-nitropyrazole-3-carboxylate (2 g, 10.803 mmol, 1 equiv) in 7M NH₃(g) in MeOH (32 mL) was stirred for overnight at 50° C. The mixture was allowed to cool down to room temperature. The resulting mixture was concentrated under reduced pressure to afford 1-methyl-4-nitropyrazole-3-carboxamide (1.8 g, 98%) as a solid. LCMS (ES, m/z): 171 [M+H]⁺

### Synthesis of Intermediate C511

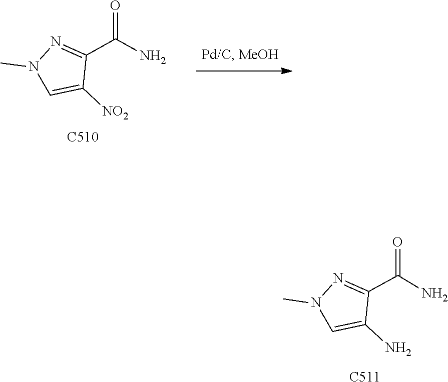

To a solution of 1-methyl-4-nitropyrazole-3-carboxamide (1.9 g, 11.168 mmol, 1 equiv) in MeOH (20 mL) was added Pd/C (10%, 1.19 g) under nitrogen atmosphere in a 50 mL round-bottom flask. The mixture was hydrogenated at room temperature for overnight under hydrogen atmosphere using a hydrogen balloon, filtered through a Celite pad and concentrated under reduced pressure to afford 4-amino-1-methylpyrazole-3-carboxamide (1.5 g, 96%) as a solid. LCMS (ES, m/z): 141 [M+H]⁺

### Synthesis of Intermediate C512

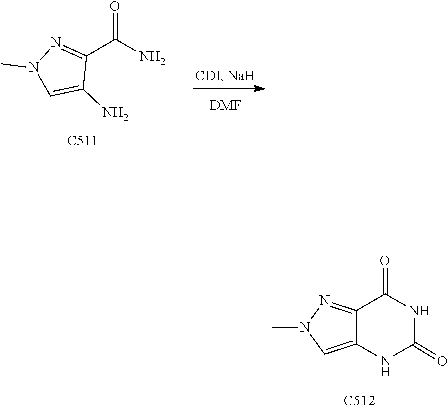

To a stirred solution of 4-amino-1-methylpyrazole-3-carboxamide (1.5 g, 10.703 mmol, 1 equiv) in dimethylformamide (20 mL) was added NaH (1.54 g, 64.218 mmol, 6 equiv) in portions at 0° C. under nitrogen atmosphere. The resulting mixture was stirred for 0.5 h at 0° C. under nitrogen atmosphere. To the above mixture was added CDI (5.21 g, 32.109 mmol, 3 equiv) at 0° C. The resulting mixture was stirred for additional 3 h at 75° C. The mixture was allowed to cool down to room temperature. The reaction was quenched by the addition of water (50 mL) at room temperature. The residue was purified by reverse flash chromatography (Condition 5, Gradient 7) to afford 2-methyl-4H,6H-pyrazolo[4,3-d]pyrimidine-5,7-dione (300 mg, 17%) as a solid. LCMS (ES, m/z): 167 [M+H]⁺

### Synthesis of Intermediate C513

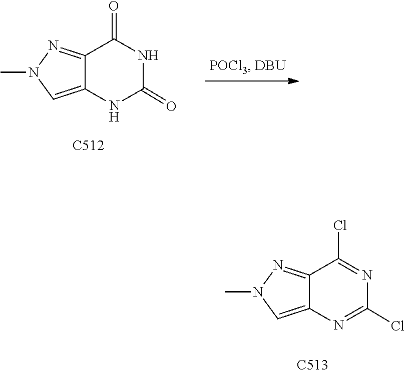

A solution of 2-methyl-4H,6H-pyrazolo[4,3-d]pyrimidine-5,7-dione (300 mg, 1.806 mmol, 1 equiv) in POCl₃ (2.8 g, 18.060 mmol, 10 equiv) was stirred for 2 h at 50° C. To the above mixture was added DBU (1.65 g, 10.836 mmol, 6 equiv) at 50° C. The resulting mixture was stirred for additional 8 h at 80° C. The mixture was allowed to cool down to room temperature. The reaction was quenched by the addition of water (20 mL) at room temperature. The mixture was basified to pH 8 with saturated aq NaHCO₃. The resulting mixture was extracted with EtOAc (3×5 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:10) to afford 5,7-dichloro-2-methylpyrazolo[4,3-d]pyrimidine (250 mg, 68%) as a solid. LCMS (ES, m/z): 203 [M+H]⁺

### Synthesis of Intermediate C514

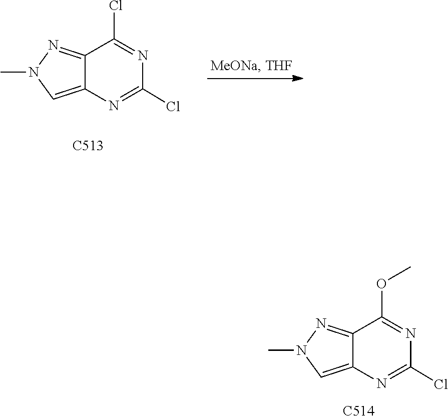

To a stirred solution of 5,7-dichloro-2-methylpyrazolo[4,3-d]pyrimidine (250 mg, 1.231 mmol, 1 equiv) in tetrahydrofuran (5 mL) was added sodium methoxide (63 mg, 1.169 mmol, 0.95 equiv) in portions at room temperature under nitrogen atmosphere. The resulting mixture was stirred for 1 h at room temperature under nitrogen atmosphere. The reaction was quenched by the addition of water (20 mL) at room temperature. The resulting mixture was extracted with EtOAc (3×5 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:1) to afford 5-chloro-7-methoxy-2-methylpyrazolo[4,3-d]pyrimidine (180 mg, 74%) as a solid. LCMS (ES, m/z): 199 [M+H]⁺

### Synthesis of Intermediate C515

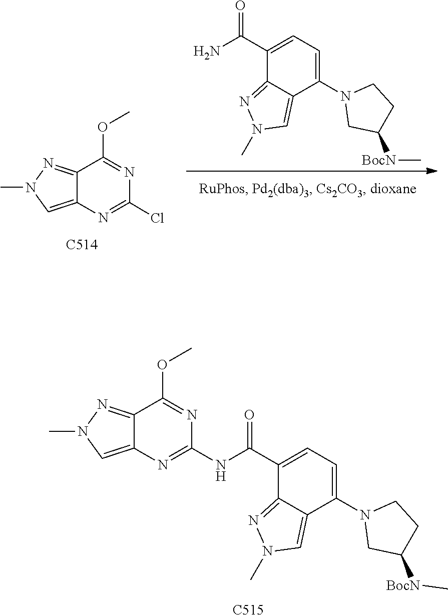

To a stirred solution of tert-butyl N-[(3R)-1-(7-carbamoyl-2-methylindazol-4-yl)pyrrolidin-3-yl]-N-methylcarbamate (100 mg, 0.268 mmol, 1 equiv) and 5-chloro-7-methoxy-2-methylpyrazolo[4,3-d]pyrimidine (53 mg, 0.268 mmol, 1 equiv) in dioxane (3 mL) were added Cs₂CO₃ (175 mg, 0.536 mmol, 2 equiv), RuPhos (25 mg, 0.054 mmol, 0.2 equiv) and Pd₂(dba)₃ (25 mg, 0.027 mmol, 0.1 equiv) at room temperature under nitrogen atmosphere. The resulting mixture was stirred for 3 h at 100° C. under nitrogen atmosphere. The mixture was allowed to cool down to room temperature. The reaction was quenched by the addition of water (20 mL) at room temperature. The resulting mixture was extracted with EtOAc (3×5 mL). The combined organic layers were washed with brine (1×5 mL), dried over anhydrous Na₂SO₄. After filtration, the filtrate was concentrated under reduced pressure. The residue was purified by reverse flash chromatography (Condition 10, Gradient 3) to afford tert-butyl N-[(3R)-1-[7-({7-methoxy-2-methylpyrazolo[4,3-d]pyrimidin-5-yl}carbamoyl)-2-methylindazol-4-yl]pyrrolidin-3-yl]-N-methylcarbamate (90 mg, 63%) as a solid. LCMS (ES, m/z): 536 [M+H]⁺

### Synthesis of Compound 624

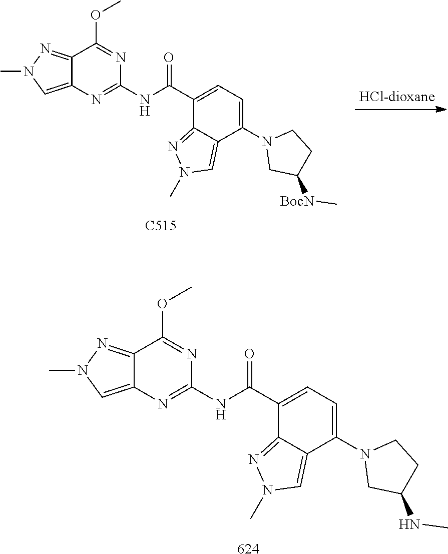

A solution of tert-butyl N-[(3R)-1-[7-({7-methoxy-2-methylpyrazolo[4,3-d]pyrimidin-5-yl}carbamoyl)-2-methylindazol-4-yl]pyrrolidin-3-yl]-N-methylcarbamate (90 mg, 0.168 mmol, 1 equiv) in 4 M HCl(gas) in 1,4-dioxane (3 mL) was stirred for 1 h at room temperature. The resulting mixture was concentrated under reduced pressure. The residue was purified by reverse flash chromatography (Condition 3, Gradient 3) to afford N-{7-methoxy-2-methylpyrazolo[4,3-d]pyrimidin-5-yl}-2-methyl-4-[(3R)-3-(methylamino)pyrrolidin-1-yl]indazole-7-carboxamide (16 mg, 19%) as a solid. LCMS (ES, m/z): 436 [M+H]^{+ 1}H NMR (300 MHz, DMSO-d₆) δ 11.50 (s, 1H), 8.83 (s, 1H), 8.40 (s, 1H), 7.95 (d, J=8.3 Hz, 1H), 6.02 (d, J=8.4 Hz, 1H), 4.24 (s, 3H), 4.17 (d, J=3.6 Hz, 6H), 3.77 (q, J=9.6 Hz, 2H), 3.66 (s, 2H), 2.35 (s, 3H), 2.21-2.09 (m, 1H), 1.95 (s, 2H).

### Example 308: Synthesis of Compound 626

### Synthesis of Intermediate C516

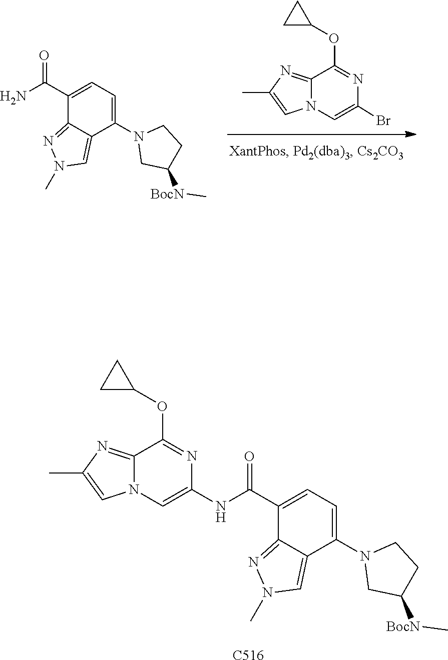

To a stirred solution of tert-butyl N-[(3R)-1-(7-carbamoyl-2-methylindazol-4-yl)pyrrolidin-3-yl]-N-methylcarbamate (100 mg, 0.268 mmol, 1 equiv) and 6-bromo-8-cyclopropoxy-2-methylimidazo[1,2-a]pyrazine (107. mg, 0.402 mmol, 1.5 equiv) in 1,4-dioxane (4.0 mL) were added Cs₂CO₃ (261 mg, 0.804 mmol, 3 equiv), RuPhos (24 mg, 0.054 mmol, 0.2 equiv) and Pd₂(dba)₃ (24 mg, 0.027 mmol, 0.1 equiv) at room temperature under nitrogen atmosphere. The resulting mixture was stirred for 3 h at 90° C. The mixture was allowed to cool down to room temperature. The residue was purified by silica gel column chromatography, eluted with EA to afford tert-butyl N-[(3R)-1-[7-({8-cyclopropoxy-2-methylimidazo[1,2-a]pyrazin-6-yl}carbamoyl)-2-methylindazol-4-yl]pyrrolidin-3-yl]-N-methylcarbamate (90 mg, 60%) as a solid. LCMS (ES, m/z): 561 [M+H]⁺

### Synthesis of Compound 626

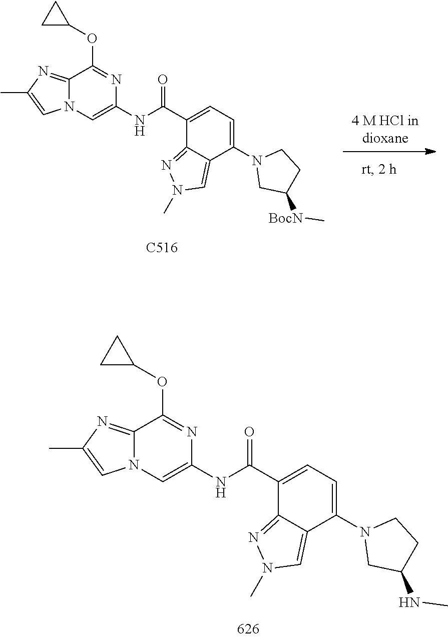

A solution of tert-butyl N-[(3R)-1-[7-({8-cyclopropoxy-2-methylimidazo[1,2-a]pyrazin-6-yl}carbamoyl)-2-methylindazol-4-yl]pyrrolidin-3-yl]-N-methylcarbamate (90 mg, 0.161 mmol, 1 equiv) in 4 M HCl (gas) in 1,4-dioxane (4 mL) was stirred for 2 h at room temperature. The resulting mixture was concentrated under reduced pressure. The residue was purified by reverse flash chromatography (Condition 3, Gradient 3) to afford N-{8-cyclopropoxy-2-methylimidazo[1,2-a]pyrazin-6-yl}-2-methyl-4-[(3R)-3-(methylamino)pyrrolidin-1-yl]indazole-7-carboxamide (22 mg, 30%) as a solid. LCMS (ES, m/z): 461 [M+H]⁺1H NMR (400 MHz, DMSO-d₆) δ 11.28 (s, 1H), 9.00 (s, 1H), 8.83 (s, 1H), 7.98-7.89 (m, 2H), 6.02 (d, J=8.4 Hz, 1H), 4.53 (tt, J=6.5, 3.3 Hz, 1H), 4.23 (s, 3H), 3.82-3.70 (m, 1H), 3.65 (d, J=7.2 Hz, 1H), 3.42 (d, J=9.5 Hz, 1H), 2.33 (d, J=7.5 Hz, 6H), 2.14 (dt, J=13.1, 6.5 Hz, 1H), 2.00 (s, 1H), 1.92 (dd, J=12.0, 6.5 Hz, 1H), 0.98-0.82 (m, 4H).

### Example 309: Synthesis of Compound 631

### Synthesis of Intermediate C517

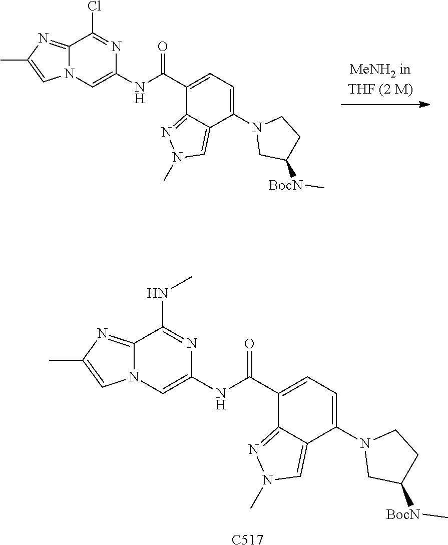

A solution of tert-butyl N-[(3R)-1-[7-({8-chloro-2-methylimidazo[1,2-a]pyrazin-6-yl}carbamoyl)-2-methylindazol-4-yl]pyrrolidin-3-yl]-N-methylcarbamate (200 mg, 0.371 mmol, 1 equiv) in 2 M methylamine in THF (2 mL) was stirred for overnight at 80° C. The mixture was allowed to cool down to room temperature. The resulting mixture was concentrated under reduced pressure. The residue was purified by silica gel column chromatography, eluted with EA to afford tert-butyl N-methyl-N-[(3R)-1-(2-methyl-7-{[2-methyl-8-(methylamino)imidazo[1,2-a]pyrazin-6-yl]carbamoyl}indazol-4-yl)pyrrolidin-3-yl]carbamate (150 mg, 76%) as a solid. LCMS (ES, m/z): 534 [M+H]⁺

### Synthesis of Compound 631

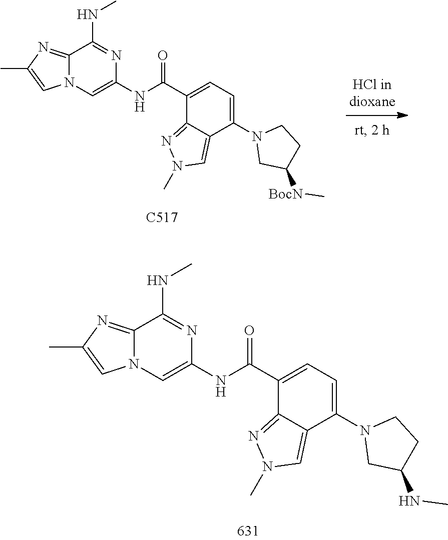

A solution of tert-butyl N-methyl-N-[(3R)-1-(2-methyl-7-{[2-methyl-8-(methylamino)imidazo[1,2-a]pyrazin-6-yl]carbamoyl}indazol-4-yl)pyrrolidin-3-yl]carbamate (90 mg, 0.169 mmol, 1 equiv) in 4 M HCl(gas) in 1,4-dioxane (2 mL) was stirred for 2 h at room temperature. The resulting mixture was concentrated under reduced pressure. The residue was purified by reverse flash chromatography (Condition 3, Gradient 3) to afford 2-methyl-N-[2-methyl-8-(methylamino)imidazo[1,2-a]pyrazin-6-yl]-4-[(3R)-3-(methylamino)pyrrolidin-1-yl]indazole-7-carboxamide (35 mg, 48%) as a solid. LCMS (ES, m/z): 434 [M+H]^{+ 1}H NMR (400 MHz, DMSO-d₆) δ 11.03 (s, 1H), 8.82 (s, 1H), 8.60 (s, 1H), 7.93 (d, J=8.3 Hz, 1H), 7.67 (d, J=1.0 Hz, 1H), 7.50 (d, J=4.9 Hz, 1H), 6.02 (d, J=8.4 Hz, 1H), 4.23 (s, 3H), 3.84-3.69 (m, 2H), 3.65 (d, J=7.4 Hz, 1H), 3.45 (d, J=9.9 Hz, 1H), 3.39 (s, 1H), 3.00 (d, J=4.7 Hz, 3H), 2.38 (s, 3H), 2.32 (s, 3H), 2.18 (dd, J=12.4, 5.9 Hz, 1H), 1.95 (dd, J=12.0, 6.4 Hz, 1H).

### Example 310: Synthesis of Compound 656

### Synthesis of Intermediate C518

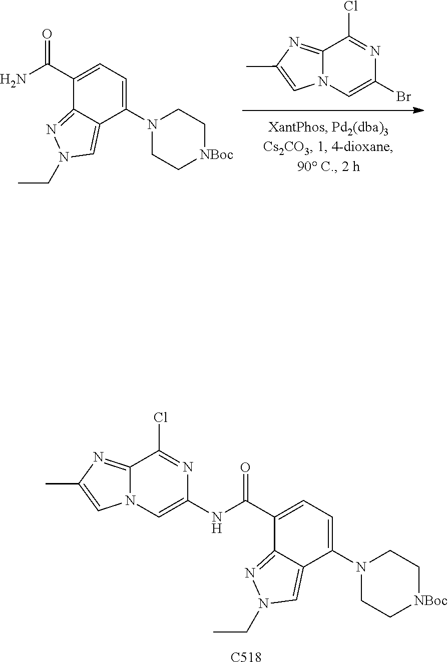

To a stirred solution of tert-butyl 4-(7-carbamoyl-2-ethylindazol-4-yl)piperazine-1-carboxylate (120 mg, 0.321 mmol, 1 equiv) and 6-bromo-8-chloro-2-methylimidazo[1,2-a]pyrazine (95 mg, 0.385 mmol, 1.2 equiv) in dioxane (2.4 mL) were added Cs₂CO₃ (314.1 mg, 0.963 mmol, 3 equiv) and XantPhos (37.2 mg, 0.064 mmol, 0.2 equiv) and Pd₂(dba)₃ (29.4 mg, 0.032 mmol, 0.1 equiv) at room temperature. The resulting mixture was stirred for 2 h at 90° C. under nitrogen atmosphere. The mixture was allowed to cool down to room temperature. The resulting mixture was diluted with water (3 mL). The resulting mixture was extracted with EtOAc (3×5 mL). The combined organic layers were washed with water (3×5 mL), dried over anhydrous Na₂SO₄. After filtration, the filtrate was concentrated under reduced pressure. The residue was purified by silica gel column chromatography, eluted with EA to afford tert-butyl 4-[7-({8-chloro-2-methylimidazo[1,2-a]pyrazin-6-yl}carbamoyl)-2-ethylindazol-4-yl]piperazine-1-carboxylate (150 mg, 87%) as a solid. LCMS (ES, m/z): 539 [M+H]⁺

### Synthesis of Compound 656

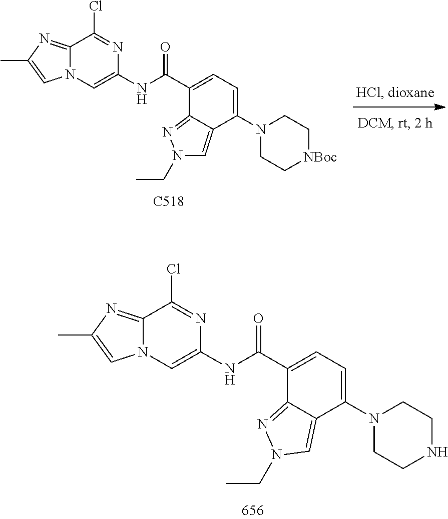

To a stirred mixture of tert-butyl 4-[7-({8-chloro-2-methylimidazo[1,2-a]pyrazin-6-yl}carbamoyl)-2-ethylindazol-4-yl]piperazine-1-carboxylate (100 mg, 0.186 mmol, 1 equiv) in DCM (1 mL) was added HCl(gas) in 1,4-dioxane (0.3 mL) dropwise at room temperature. The resulting mixture was stirred for 2 h at room temperature. The resulting mixture was concentrated under vacuum. The crude product was purified by Prep-HPLC (Condition 10, Gradient 2) to afford N-{8-chloro-2-methylimidazo[1,2-a]pyrazin-6-yl}-2-ethyl-4-(piperazin-1-yl) indazole-7-carboxamide (24 mg, 29%) as a solid. LCMS (ES, m/z): 439 [M+H]^{+ 1}H NMR (400 MHz, DMSO-d₆) δ 11.45 (s, 1H), 9.45 (s, 1H), 8.84 (s, 1H), 8.17 (s, 1H), 8.03 (d, J=8.1 Hz, 1H), 6.51 (d, J=8.2 Hz, 1H), 4.57 (q, J=7.3 Hz, 2H), 3.38 (t, J=4.9 Hz, 4H), 2.92 (dd, J=6.0, 3.5 Hz, 4H), 2.42 (s, 3H), 1.64 (t, J=7.3 Hz, 3H).

### Example 311: Synthesis of Compound 658

### Synthesis of Intermediate C519

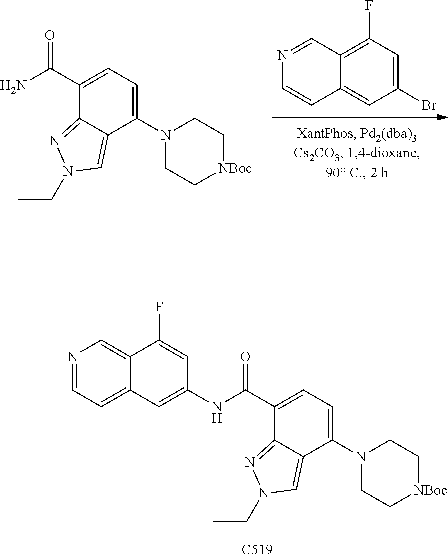

To a stirred mixture of tert-butyl 4-(7-carbamoyl-2-ethylindazol-4-yl)piperazine-1-carboxylate (100 mg, 0.268 mmol, 1 equiv) and 6-bromo-8-fluoroisoquinoline (78.7 mg, 0.348 mmol, 1.3 equiv) in dioxane (2.5 mL) were added Cs₂CO₃ (174.49 mg, 0.536 mmol, 2 equiv) and XantPhos (31 mg, 0.054 mmol, 0.2 equiv) and Pd₂(dba)₃ (24.5 mg, 0.027 mmol, 0.1 equiv) at room temperature under nitrogen atmosphere. The resulting mixture was stirred for 2 h at 90° C. under nitrogen atmosphere. The resulting mixture was diluted with water (5 mL). The resulting mixture was extracted with EtOAc (3×5 mL). The combined organic layers were washed with water (3×5 mL), dried over anhydrous Na₂SO₄. After filtration, the filtrate was concentrated under reduced pressure. The residue was purified by silica gel column chromatography, eluted with EA to afford tert-butyl 4-{2-ethyl-7-[(8-fluoroisoquinolin-6-yl)carbamoyl]indazol-4-yl}piperazine-1-carboxylate (150 mg, 97%) as a solid. LCMS (ES, m/z): 519 [M+H]⁺

### Synthesis of Compound 658

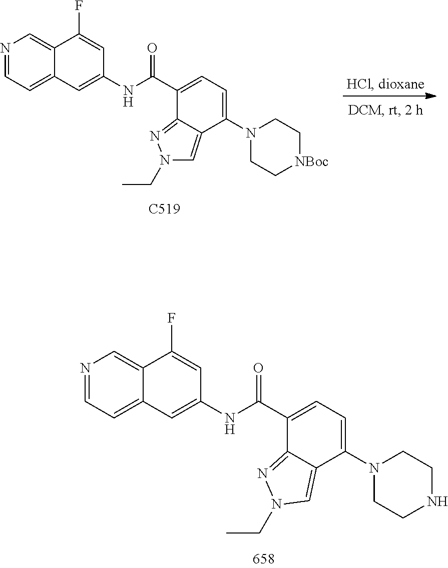

To a stirred mixture of tert-butyl 4-{2-ethyl-7-[(8-fluoroisoquinolin-6-yl)carbamoyl]indazol-4-yl}piperazine-1-carboxylate (100 mg, 0.193 mmol, 1 equiv) in DCM (1 mL) was added HCl (gas) in 1,4-dioxane (0.3 mL) dropwise room temperature. The resulting mixture was stirred for 2 h at room temperature. The resulting mixture was concentrated under reduced pressure. The crude product was purified by Prep-HPLC (Condition 10, Gradient 2) to afford 2-ethyl-N-(8-fluoroisoquinolin-6-yl)-4-(piperazin-1-yl) indazole-7-carboxamide (30 mg, 37%) as a solid. LCMS (ES, m/z): 419 [M+H]^{+ 1}H NMR (400 MHz, DMSO-d₆) δ 11.66 (s, 1H), 9.35 (s, 1H), 8.86 (s, 1H), 8.54 (d, J=5.8 Hz, 1H), 8.28 (d, J=1.8 Hz, 1H), 8.04 (d, J=8.1 Hz, 1H), 7.93 (dd, J=12.5, 1.8 Hz, 1H), 7.87 (dd, J=5.8, 1.8 Hz, 1H), 6.51 (d, J=8.2 Hz, 1H), 4.63 (q, J=7.3 Hz, 2H), 3.42-3.35 (m, 4H), 2.95-2.88 (m, 4H), 1.65 (t, J=7.3 Hz, 3H).

### Example 312: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (I)). After treatment, the levels of the HTT, MYB, or SMN2 mRNA targets were determined from each sample of cell lysate by cDNA synthesis followed by qPCR.

### Materials:

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_m1)—used for K562/suspension cell lines
GUSB: VIC-PL, ThermoFisher 4326320E (Assay: Hs99999908_m1)—used for K562/suspension cell lines
PPIA: VIC-PL, ThermoFisher 4326316E (Assay: Hs99999904_m1)—used for A673/adherent cell lines

Probe/primer sequences
Canonical junction (CJ)
HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/
Alternative junction (AJ)
HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABKFQ/
MYB Primer 1:
CAACACCATTTCATAGAGACCAGAC
MYB Primer 2:
GTTCTAAAATCATCCCTTGGCTTCTAAT
MYB FAM-Probe:
/56-FAM/AAATACTGT/ZEN/ATAGGACCTCTTCTGACATCC/
3IABKFQ/

### Description

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO₂ for 24 hours.

The cells were then gently washed with 50 uL-100 uL cold PBS before proceeding to addition of lysis buffer. 30 uL-50 uL of room temperature lysis buffer with DNAse I (and optionally RNAsin) was added to each well. Cells were shaken/mixed thoroughly at room temperature for 5-10 minutes for lysis to take place and then 3 uL-5 uL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3× lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 μL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time | |
|---|---|---|---|---|
| RT step | 1 | 50° C. | 5 | min |
| RT inactivation/initial | 1 | 95° C. | 20 | sec |
| denaturation | | | | |
| Amplification | 40 | 95° C. | 3 | sec |
| | | 60° C. | 30 | sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 A673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 500 response in AJ increase) while the decrease in CJ mRNA levels is reported as IC₅₀ (compound concentration having 50% response in CJ decrease).

A summary of these results is illustrated in Tables 3A and 3B, wherein “A” represents an AC₅₀/IC₅₀ of less than 100 Nm; “B” represents an AC₅₀/IC₅₀ of between 100 Nm and 1 μM; and “C” represents an AC₅₀/IC₅₀ of between 1 μM and 10 μM; and “D” represents an AC₅₀/IC₅₀ of greater than 10 μM.

TABLE 3A
Modulation of RNA Splicing by Exemplary Compounds

| Cmpnd | HTT | HTT | MYB | MYB |
|---|---|---|---|---|
| No. | CJ | AJ | CJ | AJ |
| 119 | C | D | C | C |
| 141 | A | A | A | A |
| 142 | C | C | B | B |
| 145 | C | C | B | B |
| 148 | C | C | C | C |
| 149 | C | C | B | B |
| 188 | D | D | C | C |
| 189 | C | C | B | B |
| 190 | B | B | A | A |
| 191 | B | C | B | B |
| 192 | C | C | B | B |
| 193 | C | C | B | B |
| 195 | D | D | B | C |
| 197 | D | D | C | C |
| 199 | B | B | B | B |
| 200 | C | C | C | C |
| 201 | D | D | D | D |
| 202 | D | C | B | A |
| 204 | C | C | B | B |
| 205 | B | C | B | B |
| 206 | C | C | B | B |
| 209 | B | B | A | A |
| 210 | B | B | A | A |
| 212 | C | D | B | C |
| 217 | D | D | D | D |
| 219 | D | D | B | C |
| 229 | B | B | A | A |
| 230 | D | D | D | D |
| 231 | D | D | D | D |
| 235 | C | C | B | B |
| 237 | C | C | B | A |
| 238 | C | C | B | B |
| 239 | B | B | A | B |
| 240 | B | B | A | A |
| 242 | B | C | A | B |
| 243 | B | B | A | A |
| 244 | D | D | D | D |
| 245 | B | B | B | B |
| 246 | C | C | A | A |
| 247 | D | D | D | D |
| 249 | D | D | B | B |
| 253 | D | D | D | D |
| 255 | C | C | B | A |
| 256 | C | C | B | B |
| 257 | C | C | B | B |
| 258 | C | B | A | A |
| 259 | C | C | B | C |
| 260 | B | B | A | A |
| 261 | D | D | D | D |
| 262 | C | C | B | B |
| 263 | C | C | B | B |
| 264 | D | D | D | D |
| 265 | D | D | D | D |
| 266 | C | C | B | A |
| 267 | D | C | C | B |
| 268 | D | D | D | D |
| 269 | B | B | A | A |
| 270 | D | D | D | D |
| 271 | D | D | C | C |
| 272 | B | B | B | A |
| 273 | D | D | B | B |
| 274 | C | D | B | B |
| 275 | C | C | B | A |
| 278 | C | C | B | A |
| 280 | C | C | D | C |
| 281 | C | C | C | C |
| 282 | C | C | D | D |
| 283 | C | D | C | D |
| 286 | D | D | B | C |
| 289 | C | D | B | B |
| 290 | D | D | B | B |
| 291 | C | C | B | B |
| 292 | C | C | A | A |
| 293 | C | B | A | A |
| 294 | B | C | A | A |
| 295 | D | C | B | B |
| 296 | D | D | A | A |
| 297 | C | C | C | C |
| 298 | C | C | A | B |
| 299 | D | D | B | B |
| 300 | D | D | C | B |
| 301 | B | B | A | A |
| 302 | C | C | A | A |
| 303 | C | C | B | B |
| 304 | C | D | B | B |
| 305 | C | C | B | B |
| 306 | C | C | B | A |
| 307 | C | C | B | B |
| 308 | D | D | D | D |
| 309 | D | D | C | D |
| 310 | C | C | A | A |
| 311 | D | D | B | C |
| 312 | C | C | B | A |
| 313 | D | D | C | C |
| 314 | C | C | A | A |
| 315 | B | B | A | A |
| 316 | D | D | C | B |
| 318 | D | D | B | B |
| 319 | C | C | A | A |
| 320 | C | C | B | B |
| 321 | D | D | D | D |
| 322 | D | D | C | D |
| 323 | A | B | A | A |
| 324 | D | C | B | B |
| 325 | D | D | C | C |
| 326 | B | B | A | A |
| 327 | D | D | B | B |
| 328 | D | D | C | D |
| 329 | D | D | D | D |
| 330 | D | D | B | B |
| 332 | D | D | D | D |
| 333 | C | C | B | B |
| 334 | D | D | B | B |
| 335 | D | D | C | C |
| 336 | B | B | A | A |
| 337 | C | C | B | B |
| 338 | C | C | B | B |
| 339 | D | D | C | C |
| 340 | D | D | C | C |
| 341 | D | D | C | C |
| 342 | D | D | C | D |
| 343 | A | A | A | A |
| 344 | D | D | C | C |
| 347 | C | C | A | A |
| 348 | C | C | A | A |
| 349 | B | B | A | A |
| 350 | C | C | A | A |
| 351 | C | C | A | A |
| 352 | B | B | A | A |
| 353 | B | B | A | A |
| 354 | D | D | C | C |
| 355 | D | C | A | A |
| 356 | D | D | B | B |
| 358 | C | D | B | B |
| 362 | D | D | C | C |
| 364 | C | D | C | B |
| 366 | C | C | A | A |
| 367 | C | C | B | B |
| 368 | D | D | B | B |
| 369 | B | B | A | A |
| 370 | D | D | B | C |
| 371 | B | B | A | A |
| 372 | C | C | A | A |
| 373 | C | C | A | A |
| 374 | D | D | A | A |
| 375 | B | B | B | B |
| 376 | B | C | B | B |
| 377 | C | C | B | B |
| 378 | C | C | B | B |
| 379 | C | D | B | C |
| 380 | C | C | A | A |
| 381 | D | D | A | A |
| 382 | C | C | A | A |
| 383 | D | D | A | B |
| 384 | D | D | D | C |
| 385 | D | D | D | D |
| 387 | C | C | A | A |
| 388 | C | C | | |
| 389 | D | D | B | C |
| 390 | D | D | D | D |
| 391 | D | C | A | B |
| 392 | C | C | A | A |
| 393 | D | D | B | B |
| 394 | C | C | B | B |
| 395 | C | C | A | A |
| 396 | D | D | B | A |
| 397 | C | C | B | B |
| 398 | D | D | B | B |
| 399 | D | D | B | B |
| 400 | D | D | A | A |
| 401 | B | B | A | A |
| 402 | B | B | A | A |
| 403 | C | C | B | B |
| 404 | | | | |
| 405 | C | C | A | B |
| 406 | B | B | A | A |
| 407 | D | D | B | B |
| 408 | D | D | B | B |
| 409 | D | D | B | A |
| 410 | D | D | B | C |
| 411 | C | C | A | A |
| 412 | C | D | A | A |
| 413 | C | C | A | A |
| 414 | D | D | C | C |
| 415 | B | B | B | B |
| 416 | D | D | D | D |
| 417 | C | D | A | A |
| 418 | D | D | B | C |
| 419 | D | D | D | D |
| 420 | D | D | B | B |
| 421 | D | D | D | D |
| 423 | B | B | A | A |
| 424 | D | D | D | D |
| 425 | D | D | D | D |
| 426 | D | D | D | D |
| 427 | B | B | B | B |
| 428 | A | A | A | A |
| 429 | C | C | B | B |
| 430 | C | C | A | A |
| 431 | D | D | B | B |
| 432 | D | D | A | A |
| 434 | C | C | B | C |
| 435 | D | D | D | D |
| 436 | D | D | B | B |
| 437 | C | C | A | A |
| 441 | C | C | A | A |
| 442 | C | D | B | B |
| 443 | C | C | A | A |
| 444 | C | D | B | B |
| 446 | C | C | B | B |
| 447 | B | B | A | A |
| 448 | B | C | A | A |
| 450 | D | D | D | D |
| 451 | D | D | C | C |
| 452 | D | D | C | C |
| 453 | D | D | C | C |
| 454 | C | C | B | C |
| 455 | D | D | D | D |
| 456 | D | D | D | C |
| 457 | D | D | D | D |
| 458 | A | B | A | A |
| 459 | B | B | A | A |
| 460 | A | B | A | A |
| 461 | C | D | A | B |
| 462 | B | B | A | A |
| 463 | B | B | A | A |
| 464 | B | B | A | A |
| 469 | D | D | D | D |
| 470 | D | D | D | D |
| 472 | D | C | A | A |
| 473 | C | C | A | A |
| 474 | A | B | A | A |
| 475 | B | B | A | A |
| 476 | B | C | C | C |
| 477 | C | D | B | B |
| 478 | C | D | A | B |
| 479 | C | C | A | A |
| 481 | B | C | A | B |
| 482 | D | D | C | C |
| 484 | D | D | D | D |
| 486 | B | B | B | B |
| 487 | C | C | C | C |
| 488 | D | D | D | D |
| 489 | D | D | D | D |
| 490 | D | D | C | C |
| 491 | C | D | B | C |
| 492 | C | D | B | C |
| 494 | D | D | D | D |
| 496 | D | D | D | D |
| 497 | B | B | A | A |
| 498 | B | C | B | B |
| 499 | D | D | D | D |
| 500 | D | D | D | D |
| 501 | A | A | A | A |
| 502 | A | B | A | A |
| 503 | B | A | A | A |
| 504 | C | C | C | C |
| 505 | C | C | B | B |
| 506 | B | C | B | B |
| 507 | C | C | B | B |
| 508 | A | B | A | A |
| 509 | D | D | C | C |
| 510 | C | C | B | B |
| 511 | B | B | B | B |
| 513 | C | D | B | B |
| 514 | B | B | A | A |
| 515 | B | B | A | A |
| 517 | C | D | B | B |
| 518 | C | C | B | A |
| 520 | C | C | A | A |
| 522 | C | C | A | B |
| 524 | D | D | A | A |
| 525 | B | B | A | A |
| 526 | D | D | C | C |
| 527 | D | D | B | B |
| 528 | D | D | B | B |
| 529 | A | B | A | A |
| 531 | C | C | B | C |
| 533 | C | C | A | A |
| 535 | D | D | B | B |
| 537 | D | D | C | C |
| 538 | C | C | C | C |
| 539 | D | D | C | C |
| 541 | D | D | B | B |
| 542 | C | C | B | B |
| 543 | C | C | A | A |
| 544 | D | D | C | C |
| 545 | C | D | B | B |
| 547 | D | D | D | D |
| 548 | D | D | D | D |
| 550 | D | D | C | C |
| 552 | B | B | B | B |
| 554 | D | D | B | A |
| 556 | D | D | C | D |
| 557 | B | B | A | A |
| 559 | C | C | A | A |
| 560 | C | C | B | B |
| 562 | D | D | C | C |
| 563 | B | B | A | A |
| 564 | B | B | A | A |
| 565 | D | D | D | D |
| 566 | C | C | B | B |
| 568 | C | C | C | C |
| 569 | D | C | A | A |
| 571 | B | B | A | A |
| 573 | D | D | B | B |
| 574 | D | D | B | B |
| 575 | D | D | D | D |
| 576 | C | D | B | B |
| 577 | B | B | B | B |
| 579 | D | D | D | D |
| 580 | D | D | B | C |
| 581 | C | C | B | B |
| 582 | B | B | B | B |
| 584 | C | C | B | B |
| 585 | D | D | B | B |
| 587 | B | B | B | B |
| 589 | D | D | B | C |
| 590 | C | C | B | B |
| 592 | C | D | B | B |
| 594 | D | D | B | C |
| 596 | D | D | C | C |
| 598 | C | C | A | B |
| 600 | D | D | C | C |
| 602 | C | C | A | B |
| 603 | D | D | C | D |
| 604 | D | D | C | C |
| 606 | B | C | B | B |
| 607 | C | C | B | B |
| 610 | D | C | B | B |
| 612 | D | D | D | D |
| 614 | D | D | B | B |
| 616 | B | C | B | B |
| 617 | C | D | B | C |
| 619 | D | D | B | B |
| 621 | D | D | C | B |
| 622 | D | D | C | D |
| 624 | C | C | C | C |
| 626 | C | C | B | B |
| 627 | C | C | B | B |
| 629 | C | C | C | C |
| 631 | D | D | C | C |
| 633 | C | D | B | B |
| 635 | D | D | B | C |
| 637 | C | C | B | C |
| 639 | D | D | B | B |
| 641 | C | C | A | A |
| 643 | D | D | B | B |
| 645 | C | D | B | B |
| 649 | D | D | B | B |
| 651 | D | D | D | D |
| 653 | D | D | D | D |

TABLE 3B

| Cmpnd | HTT | HTT | MYB | MYB |
|---|---|---|---|---|
| No. | CJ | AJ | CJ | AJ |
| 908 | B | B | A | A |
| 916 | B | B | A | A |
| 932 | D | D | C | C |
| 940 | A | B | A | A |
| 941 | B | B | A | A |
| 942 | B | C | B | B |
| 943 | B | C | A | A |
| 944 | C | C | B | B |
| 945 | C | C | A | A |
| 946 | A | B | A | A |
| 949 | A | A | A | A |
| 950 | B | B | A | B |
| 952 | B | B | B | B |
| 953 | C | C | B | B |
| 954 | C | C | B | B |
| 955 | B | C | A | A |
| 956 | A | B | A | A |
| 958 | B | C | A | A |
| 959 | B | B | A | A |
| 960 | B | B | B | B |
| 961 | B | B | A | A |
| 963 | C | C | B | B |
| 965 | A | A | A | A |
| 966 | A | A | A | A |
| 968 | B | C | A | B |
| 969 | C | D | A | A |
| 971 | B | B | A | A |
| 973 | C | C | A | A |
| 974 | B | B | A | A |
| 976 | B | C | A | A |
| 978 | B | B | A | A |
| 979 | B | B | A | A |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for three exemplary genes (HTT, SMN2, and Target C) analyzed in this panel are reported as IC₅₀ (compound concentration having 5000 response in CJ decrease).

A summary of the results from the panel is illustrated in Tables 4A and 4B3, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater than 10 μM.

TABLE 4A
Modulation of RNA Splicing by Exemplary Compounds

| Compound | | | Target | |
|---|---|---|---|---|
| No. | HTT | SMN2 | C | MYB |
| 118 | C | B | D | B |
| 119 | C | B | D | C |
| 140 | C | B | C | C |
| 141 | A | A | B | A |
| 142 | C | A | B | B |
| 143 | A | A | B | A |
| 145 | C | A | B | B |
| 148 | C | B | C | C |
| 149 | C | A | D | B |
| 187 | B | A | C | B |
| 188 | D | B | C | C |
| 189 | C | A | C | B |
| 190 | B | B | C | A |
| 191 | B | A | C | B |
| 192 | C | A | C | B |
| 193 | C | A | D | B |
| 194 | C | A | D | B |
| 195 | D | A | D | B |
| 196 | C | A | C | B |
| 197 | D | B | D | C |
| 198 | C | A | C | A |
| 199 | B | A | C | B |
| 200 | C | A | C | C |
| 201 | D | C | D | D |
| 202 | D | A | D | B |
| 203 | D | A | D | B |
| 204 | C | A | C | B |
| 205 | B | A | C | B |
| 206 | C | A | D | B |
| 207 | B | A | C | A |
| 208 | B | A | B | A |
| 209 | B | C | B | A |
| 210 | B | A | C | A |
| 211 | C | A | C | B |
| 212 | C | B | D | B |
| 217 | D | C | D | D |
| 218 | C | A | C | B |
| 219 | D | B | D | B |
| 228 | B | A | B | A |
| 229 | B | A | C | A |
| 230 | D | D | D | D |
| 231 | D | D | D | D |
| 234 | C | A | C | A |
| 235 | C | A | D | B |
| 237 | C | A | D | B |
| 238 | C | A | D | B |
| 239 | B | A | C | A |
| 240 | B | A | C | A |
| 241 | B | A | C | B |
| 242 | B | A | C | A |
| 243 | B | A | C | A |
| 244 | D | B | D | D |
| 245 | B | A | B | B |
| 246 | C | A | D | A |
| 247 | D | D | D | D |
| 249 | D | A | D | B |
| 250 | A | A | B | A |
| 251 | B | A | C | A |
| 252 | B | A | B | A |
| 253 | D | C | D | D |
| 255 | C | A | D | B |
| 256 | C | A | D | B |
| 257 | C | A | D | B |
| 258 | C | A | C | A |
| 259 | C | A | C | B |
| 260 | B | C | C | A |
| 261 | D | C | D | D |
| 262 | C | A | D | B |
| 263 | C | A | C | B |
| 264 | D | D | D | D |
| 265 | D | C | D | D |
| 266 | C | A | D | B |
| 267 | D | A | D | C |
| 268 | D | D | D | D |
| 269 | B | A | C | A |
| 270 | D | D | D | D |
| 271 | D | B | D | C |
| 272 | B | A | B | B |
| 273 | D | A | D | B |
| 274 | C | A | D | B |
| 275 | C | A | D | B |
| 277 | B | A | C | A |
| 278 | C | A | C | B |
| 279 | C | A | C | A |
| 280 | C | C | D | D |
| 281 | C | B | C | C |
| 282 | C | B | D | D |
| 283 | C | B | D | C |
| 284 | C | A | C | A |
| 285 | B | A | B | A |
| 286 | D | B | D | B |
| 288 | C | A | C | A |
| 289 | C | A | D | B |
| 290 | D | A | D | B |
| 291 | C | A | C | B |
| 292 | C | A | C | A |
| 293 | C | A | D | A |
| 294 | B | A | D | A |
| 295 | D | A | D | B |
| 296 | D | A | D | A |
| 297 | C | B | C | C |
| 298 | C | A | C | A |
| 299 | D | A | D | B |
| 300 | D | B | D | C |
| 301 | B | A | C | A |
| 302 | C | A | D | A |
| 303 | C | A | D | B |
| 304 | C | A | D | B |
| 305 | C | A | D | B |
| 306 | C | A | D | B |
| 307 | C | A | D | B |
| 308 | D | D | D | D |
| 309 | D | C | D | C |
| 310 | C | A | D | A |
| 311 | D | A | D | B |
| 312 | C | A | C | B |
| 313 | D | B | C | C |
| 314 | C | A | D | A |
| 315 | B | A | B | A |
| 316 | D | B | D | C |
| 318 | D | A | D | B |
| 319 | C | A | C | A |
| 320 | C | A | D | B |
| 321 | D | D | D | D |
| 322 | D | C | D | C |
| 323 | A | A | C | A |
| 324 | D | A | D | B |
| 325 | D | A | D | C |
| 326 | B | A | C | A |
| 327 | D | B | D | B |
| 328 | D | B | D | C |
| 329 | D | C | D | D |
| 330 | D | A | D | B |
| 332 | D | D | D | D |
| 333 | C | A | D | B |
| 334 | D | A | D | B |
| 335 | D | B | D | C |
| 336 | B | A | B | A |
| 337 | C | A | D | B |
| 338 | C | A | D | B |
| 339 | D | B | D | C |
| 340 | D | B | D | C |
| 341 | D | B | D | C |
| 342 | D | D | D | C |
| 343 | A | A | B | A |
| 344 | D | B | D | C |
| 347 | C | A | C | A |
| 348 | C | A | C | A |
| 349 | B | A | B | A |
| 350 | C | A | D | A |
| 351 | C | A | D | A |
| 352 | B | A | C | A |
| 353 | B | A | C | A |
| 354 | D | B | D | C |
| 355 | D | A | D | A |
| 356 | D | A | D | B |
| 358 | C | A | D | B |
| 361 | A | A | A | A |
| 362 | D | B | D | C |
| 363 | A | A | A | A |
| 364 | C | A | D | C |
| 366 | C | A | D | A |
| 367 | C | A | D | B |
| 368 | D | A | D | B |
| 369 | B | A | C | A |
| 370 | D | B | D | B |
| 371 | D | A | D | A |
| 372 | D | A | D | A |
| 373 | C | A | D | A |
| 374 | D | A | D | A |
| 375 | B | A | B | B |
| 376 | C | A | D | A |
| 377 | C | A | C | A |
| 378 | C | A | D | A |
| 379 | C | A | C | B |
| 380 | C | A | C | A |
| 381 | D | A | D | A |
| 382 | C | A | D | A |
| 383 | D | A | D | A |
| 384 | D | B | D | D |
| 385 | D | D | D | D |
| 386 | D | D | D | D |
| 387 | C | A | D | A |
| 388 | D | A | D | A |
| 389 | D | A | D | B |
| 407 | D | A | D | B |
| 408 | D | A | D | B |
| 409 | D | A | D | B |
| 410 | D | A | D | B |
| 411 | D | A | D | A |
| 412 | C | A | D | A |
| 413 | C | A | D | A |
| 414 | D | C | D | C |
| 415 | B | B | B | B |
| 416 | D | C | D | D |
| 417 | D | A | D | A |
| 418 | D | A | D | B |
| 419 | D | D | D | D |
| 420 | D | A | D | B |
| 421 | D | D | D | D |
| 422 | A | A | B | A |
| 423 | B | A | C | A |
| 424 | D | C | D | D |
| 425 | D | D | D | D |
| 426 | D | D | D | D |
| 427 | B | A | C | B |
| 428 | A | A | B | A |
| 429 | C | A | D | B |
| 430 | C | A | D | A |
| 431 | C | A | C | A |
| 432 | D | A | D | B |
| 433 | C | A | C | B |
| 434 | D | A | D | A |
| 435 | C | A | C | B |
| 436 | D | D | D | D |
| 437 | D | A | D | B |
| 440 | A | A | C | A |
| 441 | C | A | D | A |
| 442 | C | A | D | A |
| 443 | C | A | D | B |
| 444 | C | A | C | A |
| 445 | A | A | B | A |
| 446 | C | A | C | B |
| 447 | C | A | C | B |
| 448 | B | A | C | A |
| 449 | C | A | D | A |
| 450 | B | A | C | A |
| 451 | D | D | D | D |
| 452 | D | B | D | C |
| 453 | D | B | D | C |
| 454 | D | A | D | C |
| 455 | C | B | C | B |
| 456 | D | D | D | D |
| 457 | D | D | D | D |
| 458 | D | D | D | D |
| 459 | A | A | C | A |
| 460 | B | A | B | A |
| 461 | C | A | D | A |
| 462 | B | A | D | A |
| 463 | B | A | D | A |
| 464 | B | A | C | A |
| 469 | D | D | D | D |
| 470 | D | D | D | D |
| 471 | A | A | A | A |
| 472 | D | A | D | A |
| 473 | C | A | D | A |
| 474 | B | A | C | A |
| 475 | B | A | C | A |
| 476 | B | B | C | C |
| 477 | C | B | D | B |

TABLE 4B

| Compound | | | Target | |
|---|---|---|---|---|
| No. | HTT | SMN2 | C | MYB |
| 908 | B | A | C | A |
| 916 | B | A | B | A |
| 932 | D | B | D | C |
| 940 | A | A | B | A |
| 941 | B | A | C | A |
| 942 | B | A | C | B |
| 943 | B | A | C | A |
| 944 | C | A | C | B |
| 945 | C | A | C | A |
| 946 | A | A | B | A |
| 949 | A | A | A | A |
| 950 | B | A | D | A |
| 952 | B | A | B | B |
| 953 | C | A | C | B |
| 954 | C | A | C | B |
| 955 | B | A | D | A |
| 956 | A | A | B | A |
| 958 | B | A | D | A |
| 959 | B | A | B | A |
| 960 | B | A | B | B |
| 961 | B | A | B | A |
| 963 | C | A | D | B |
| 965 | A | A | B | A |
| 966 | A | A | A | A |
| 968 | B | A | C | A |
| 969 | C | A | C | A |
| 971 | B | A | B | A |
| 973 | C | A | C | A |
| 974 | B | A | B | A |
| 976 | B | A | C | A |
| 978 | B | A | C | A |
| 979 | B | A | C | A |

### Example 313: Evaluating Effect of Exemplary Compounds on Protein Abundance

Compounds described herein were used to screen for effects on quantitative protein abundance using a HiBit assay system (Promega). Quantitative protein abundance was determined by measuring the protein levels of HiBit-tagged protein targets expressed in cell culture via luminescence using the Nano-Glo HiBiT Lytic Detection System, which uses a split complementation assay format to reconstitute NanoBiT enzyme to generate a luminescent signal. A protein abundance assay was developed such that endogenous protein targets could be modified with the HiBiT peptide tag and their abundance could be assessed after compound treatment. Briefly, K562 cell lines containing a HiBiT-modification were treated with various compounds described herein (e.g., compounds of Formulas (I) or (II)). After treatment for 24 hours, the protein abundance of a specific target was determined by measuring luminescence.

### Materials:

Promega Nano-Glo HiBiT Lytic Detection System (cat #N3030)
Corning 384-well TC-treated microplates (cat #3570)
Synthego Engineered Cells Knock-In Clones

TABLE 5
Design of genetically modified HiBiT cell lines

| | | | Guide | Guide | |
|---|---|---|---|---|---|
| Cell | | | RNA | RNA cut | |
| Line | Gene | Modification | Sequence | location | Donor Sequence |
| K562 | MYB | HiBiT | GCGCCA | chr6: | CGGTGCGGTCCCCGCGGCTC |
| | | | TGGCCC | 135,181,526 | TCGGCGGAGCCCCGCGCCCG |
| | | | GAAGAC | | CCGCGCCATGgtgagcggctggcgg |
| | | | CC | | ctgttcaagaagattagcGGCAGCTCC |
| | | | | | GGAGGATCTAGCGGCGCCCG |
| | | | | | AAGACCCCGGCACAGgtaacgg |
| | | | | | ggagccggggggcggccgaggg |
| K562 | HTT | HiBiT | CAGCTTT | chr4: | CGAGTCGGCCCGAGGCCTCC |
| | | | TCCAGG | 3,074,830 | GGGGACTGCCGTGCCGGGCG |
| | | | GTCGCC | | GGAGACCGCCATGgtgagcggctg |
| | | | A | | gcggctgttcaagaagattagcGGCAGC |
| | | | | | TCCGGAGGATCTAGCGGCGC |
| | | | | | GACCCTGGAAAAGCTGATGA |
| | | | | | AGGCCTTCGAGTCCCTCAAG |
| | | | | | TCCTTCCA |

### Description:

Cells were maintained in EIDM with 10% FBS. Before the assay, cells were diluted with phenolphthalein-free growth media (EIDM+1% FBS media) and were seeded in a 384-well plate at a density of 10000 cells/well (for each cell line listed in Table 5). Each compound was prepared as a 10-point 3-fold serial dilution in DMSO with the top dose at a final concentration of 10 μM in the well. Unmodified K562 cells were added at the previously specified density with DMSO to serve as an assay baseline and positive control (PC) and DMSO only with the respective modified cell lines was added to the negative control (NC) columns. Final DMSO concentration was kept at or below 0.25%. Treated cell plates were placed in an incubator at 37° C. with 5% CO₂ for 24 hours. After 24 hours, 25 μL of Complete HiBit Lytic reagent was added to each well at room temperature (e.g. one plate requiring 10 mL Lytic Buffer, 100 μL LgBiT Protein, 200 μL Lytic Substrate), shaken for 5 minutes at 600 RPM, then left to sit for 10 minutes for signal to stabilize before reading on a Spark Cyto plate reader (Tecan) with a 500 ms measurement time.

To determine compound effects on protein abundance of each target in Table 6, the percent response for each respective cell line was calculated at each compound concentration as follows:

$$\% response = 100 * ( S - PC ) / ( NC - PC )$$

For the normalized response at each concentration, a four-parameter logistical regression was fit to the data and the response was interpolated at the 50% value to determine a concentration for protein abundance at 5000 (IC₅₀) the untreated control.

A summary of the results for protein abundance is illustrated in Table 6, wherein A represents <100 nM; B represents 100-1000 nM; C represents 1000-9999 nM; and D represents greater than 10 μM.

TABLE 6

| Compound No. | HTT | MYB | Target C |
|---|---|---|---|
| 118 | B | A | C |
| 119 | C | C | D |
| 140 | C | C | C |
| 141 | B | B | C |
| 142 | B | A | C |
| 143 | A | A | A |
| 145 | C | A | C |
| 148 | C | C | C |
| 149 | C | A | C |
| 187 | A | B | B |
| 189 | D | B | C |
| 190 | B | A | C |
| 191 | B | A | C |
| 192 | C | B | C |
| 193 | C | B | D |
| 194 | C | B | D |
| 195 | D | C | C |
| 196 | B | A | C |
| 197 | D | C | D |
| 198 | C | A | D |
| 199 | C | A | D |
| 200 | C | C | C |
| 187 | C | A | C |
| 190 | B | A | C |
| 191 | C | B | C |
| 192 | C | B | C |
| 193 | C | B | D |
| 194 | C | B | D |
| 197 | D | C | D |
| 198 | C | A | D |
| 199 | C | A | D |
| 200 | A | A | B |
| 208 | B | A | B |
| 217 | D | D | D |
| 556 | C | C | C |
| 557 | B | A | B |
| 559 | B | A | B |
| 560 | C | A | C |
| 562 | D | D | D |
| 563 | B | A | B |
| 564 | B | B | B |
| 565 | C | C | D |
| 566 | C | B | C |
| 568 | C | C | C |
| 569 | C | A | C |
| 571 | B | A | C |
| 573 | D | C | D |
| 574 | D | B | D |
| 575 | C | C | D |

### Example 314: Investigating Effect of Exemplary Compounds on Cell Viability

Compounds described herein were screened for toxicity in K562 (human chronic myelogenous leukemia) and SH-SY5Y (human neuroblastoma) cells using a Cell Titer Glo 2.0 assay.

### Materials:

Promega CellTiter-Glo® 2.0 Cell Viability Assay (cat #G9241)
Corning 384-well TC-treated microplates (cat #3570)

### Description:

Cells were plated at 500 cells/well (K562 cells) in 45 μL of IMDM supplemented with 10% FBS in a 384-well opaque plate. Wells containing only medium were used as a blank control. Test compounds (e.g., compounds of Formula (I) or (II)) were first serially diluted in DMSO then diluted 1:100 with EIDM+10% FBS. The final concentration of DMSO was 0.1% in each well. The cells were incubated for 72 hours at 37° C. and 5% CO₂ before assaying with Cell Titer Glo 2.0 reagent.

The compounds tested exhibited the following range as shown in Table 7, wherein A represents <100 nM; B represents 100-1000 nM; C represents 1000-9999 nM; and D represents greater than 10 μM in K562 cells.

TABLE 7

| Compound No. | GI₅₀ nM |
|---|---|
| 118 | B |
| 119 | C |
| 140 | C |
| 141 | B |
| 142 | B |
| 143 | B |
| 148 | C |
| 149 | B |
| 187 | B |
| 189 | B |
| 190 | B |
| 191 | B |
| 192 | B |
| 193 | B |
| 194 | B |
| 195 | B |
| 196 | A |
| 197 | B |
| 198 | A |
| 199 | B |
| 200 | C |
| 208 | B |
| 217 | B |
| 556 | B |
| 557 | A |
| 559 | A |
| 560 | A |
| 562 | C |
| 563 | B |
| 564 | B |
| 565 | C |
| 566 | B |
| 568 | B |
| 569 | B |
| 571 | B |
| 573 | C |
| 574 | B |
| 575 | B |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (I): 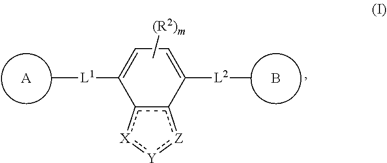
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
X, Y, and Z are each independently C(R^{3a}), C(R^{3a})(R^{3b}), N, N(R^{3c}), or O, wherein at least one of X, Y, and Z is N, N(R^{3c}), or O, and the bonds in the ring comprising X, Y, and Z may be single or double bonds as valency permits;
each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶;
each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}
R^{3a} and R^{3b} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; or
each of R^{3a} and R^{3b}, together with the carbon atom to which they are attached, form an oxo group;
R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R′;
each R⁴ is independently hydrogen, C₁-C₆-alkyl, C₁-C₆-heteroalkyl, or C₁-C₆-haloalkyl;
each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹;
each R⁷ is C₁-C₆-alkyl, halo, cyano, oxo, or —OR^{A1};
each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R²;
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R¹² is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₂-C₆ alkenyl, C₂-C₆ alkynyl, C₁-C₆ haloalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or
R^{B} and R^{C} together with the atom to which they are attached form a 3-7-membered heterocyclyl ring optionally substituted with one or more R⁷;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R^{A1} is hydrogen or C₁-C₆-alkyl;
m is 0, 1, or 2; and
x is 0, 1, or 2.

2. The compound of claim 1, wherein each of A and B is independently heteroaryl or heterocyclyl, each of which is optionally substituted with one or more R¹.

3. The compound of any one of the preceding claims, wherein one of A and B is independently a monocyclic heteroaryl or bicyclic heteroaryl, each of which is optionally substituted with one or more R¹.

4. The compound of any one of the preceding claims, wherein one of A and B is independently a bicyclic heteroaryl optionally substituted with one or more R¹.

5. The compound of any one of the preceding claims, wherein one of A and B is independently a nitrogen-containing heteroaryl optionally substituted with one or more R¹.

6. The compound of any one of the preceding claims, wherein one of A and B is a 5-10 membered heteroaryl optionally substituted with one or more R¹.

7. The compound of any one of the preceding claims, wherein one of A and B is independently selected from 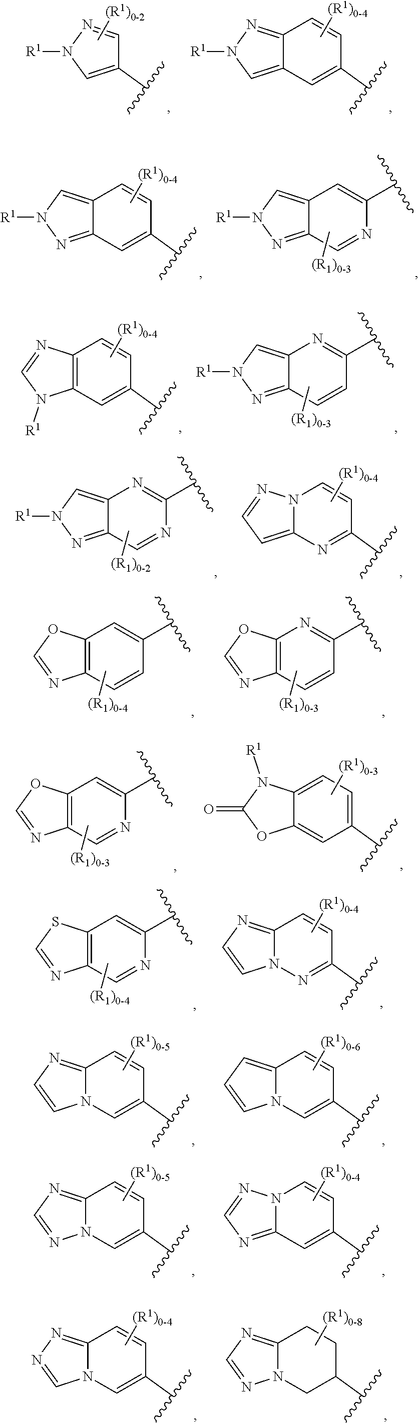 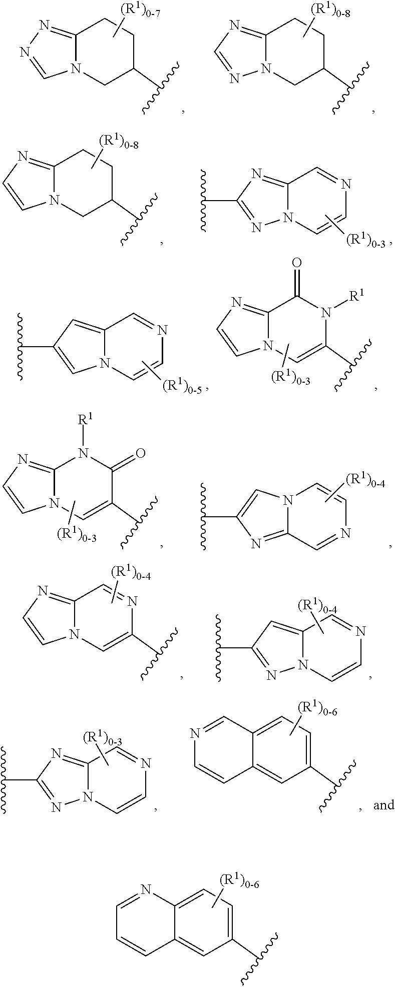
wherein R¹ is as described in claim 1.

8. The compound of any one of the preceeding claims, wherein one of A and B is independently selected from 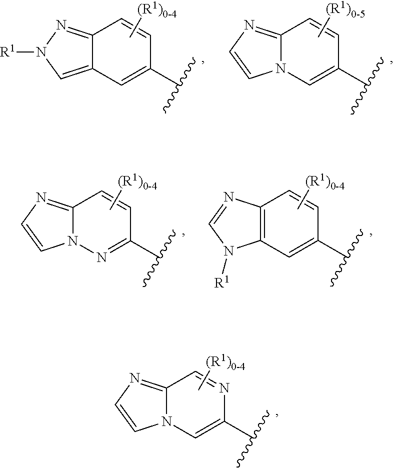
wherein R¹ is as described in claim 1.

9. The compound of any one of the preceding claims, wherein one of A and B is independently selected from 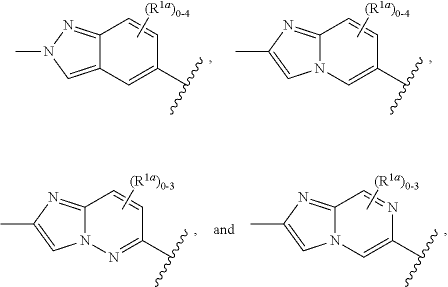
wherein each Ria is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

10. The compound of any one of the preceding claims, wherein one of A and B is independently 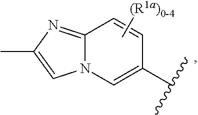
wherein each Ria is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

11. The compound of any one of the preceding claims, wherein A is selected from 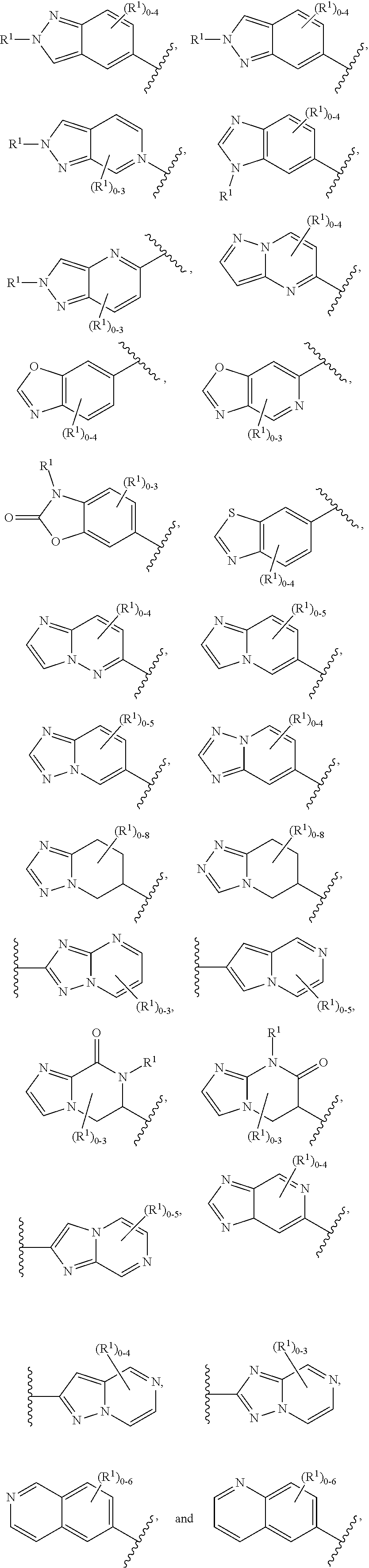
wherein R¹ is as described in claim 1, wherein R¹ is as described in claim 1.

12. The compound of any one of the preceding claims, wherein B is selected from 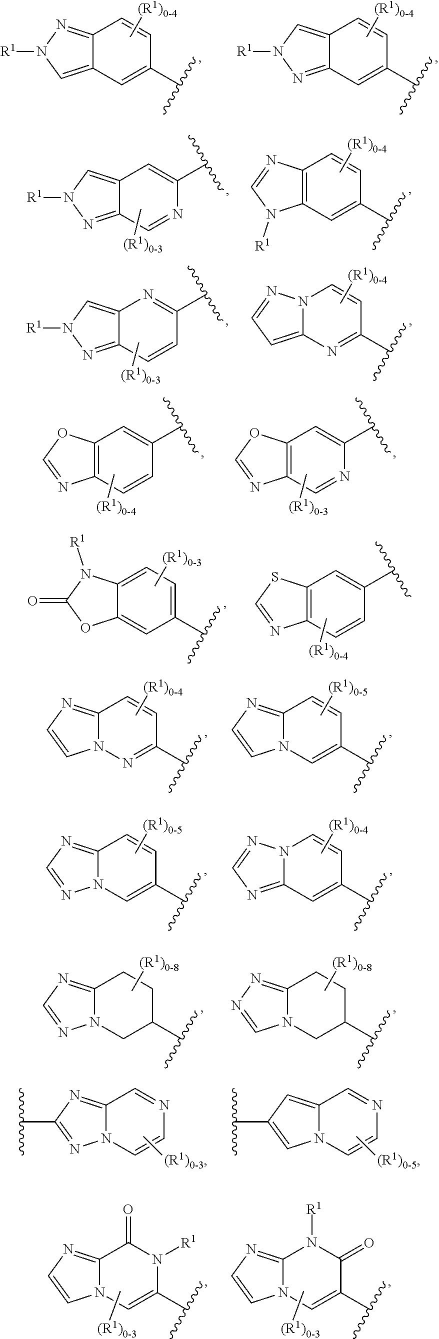 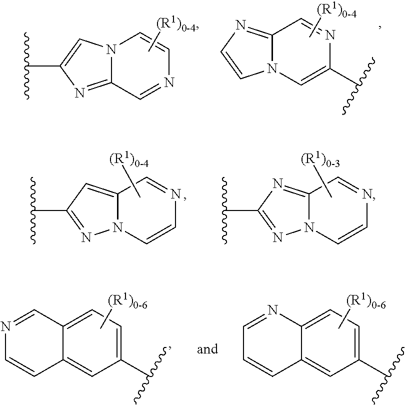
wherein R¹ is as described in claim 1, wherein R¹ is as described in claim 1.

13. The compound of any one of the preceding claims, wherein A is selected from 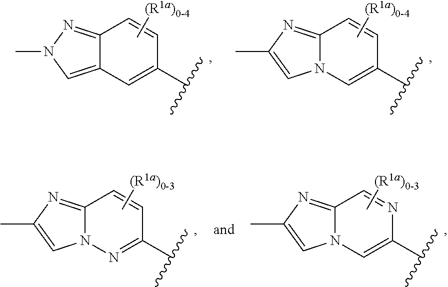
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

14. The compound of any one of the preceding claims, wherein A is 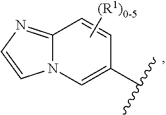
wherein R¹ is as described in claim 1.

15. The compound of any one of the preceding claims, wherein one of A and B is independently selected from

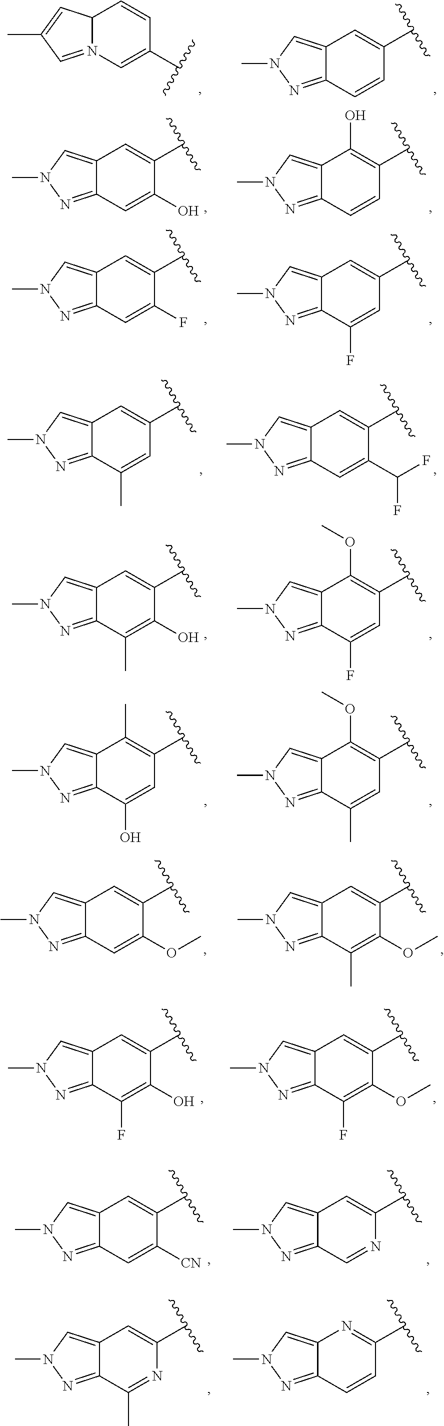

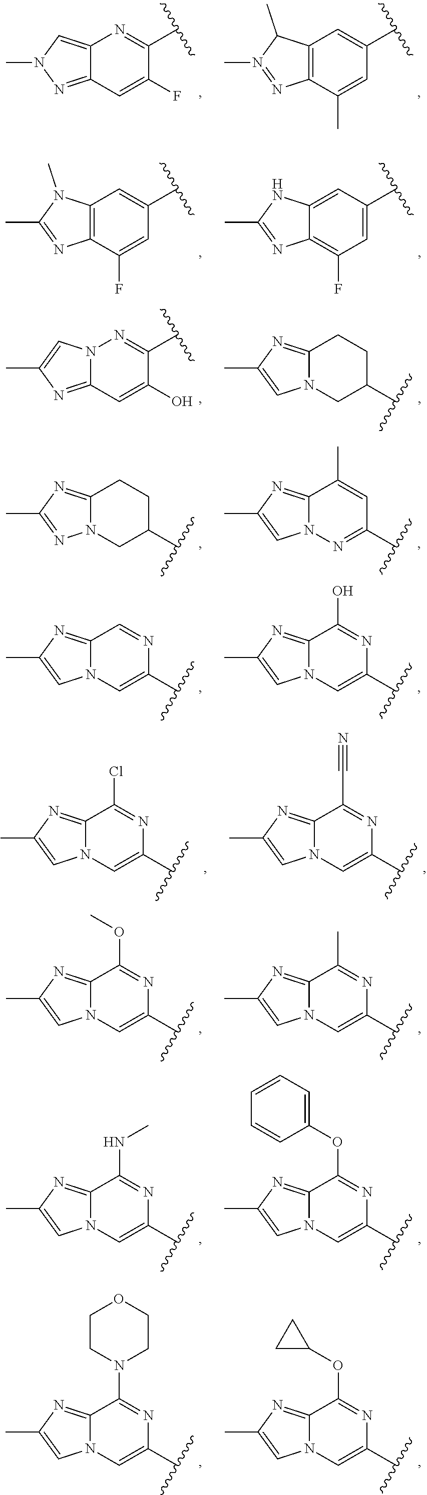

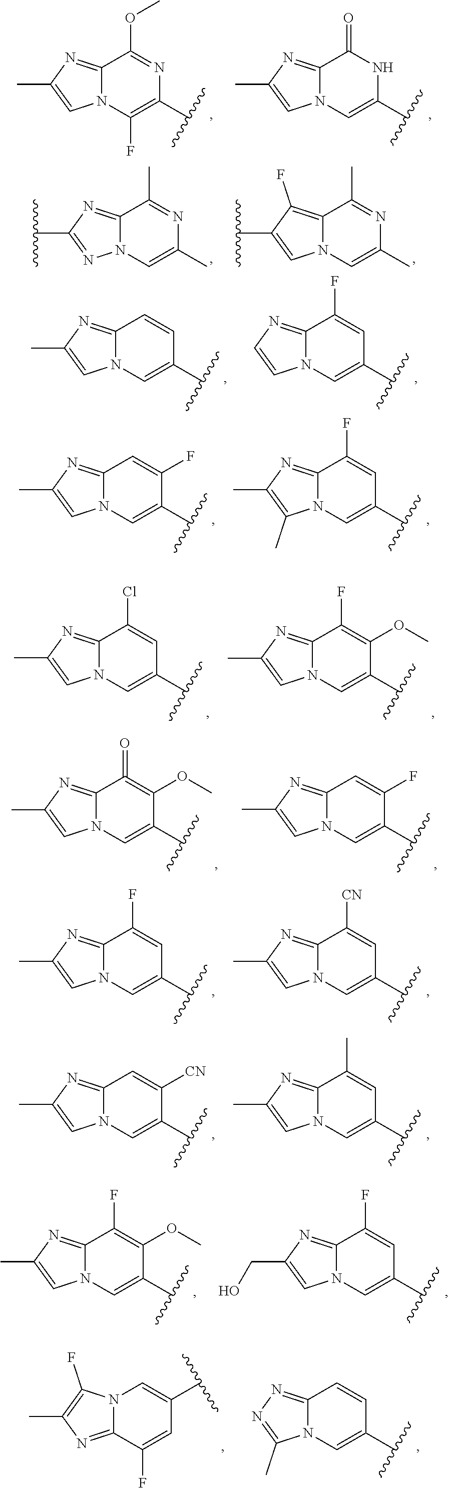

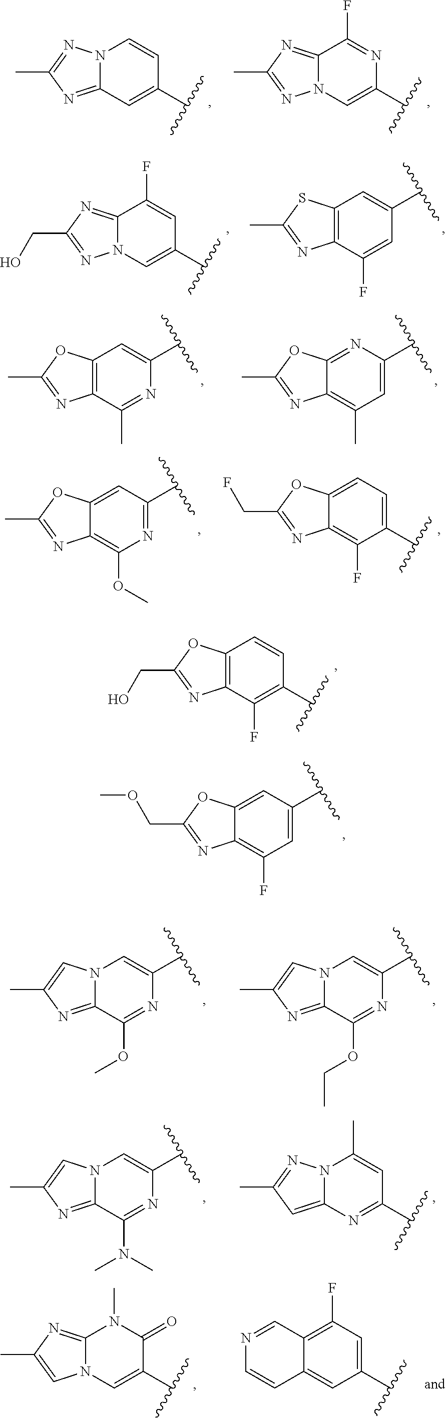

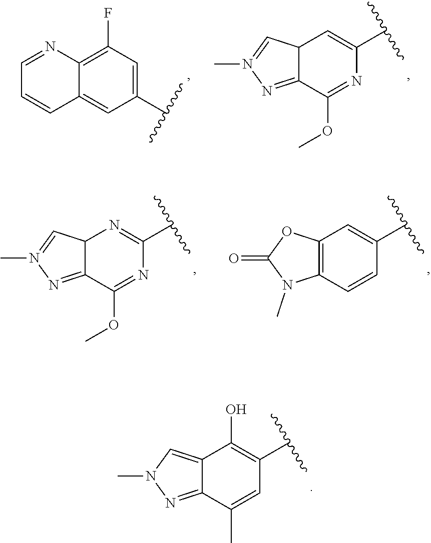

16. The compound of any one of the preceding claims, wherein one of A and B is independently selected from

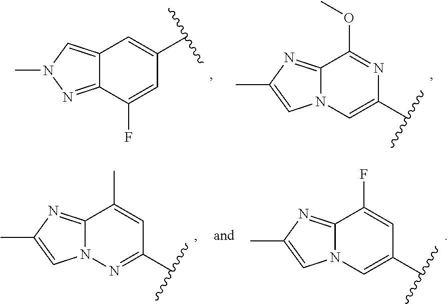

17. The compound of any one of the preceding claims, wherein one of A and B is independently

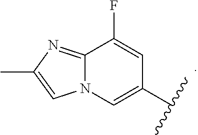 [SMILES: *c1cc(F)c2nc(C)cn2c1]

18. The compound of any one of the preceding claims, wherein A is selected from

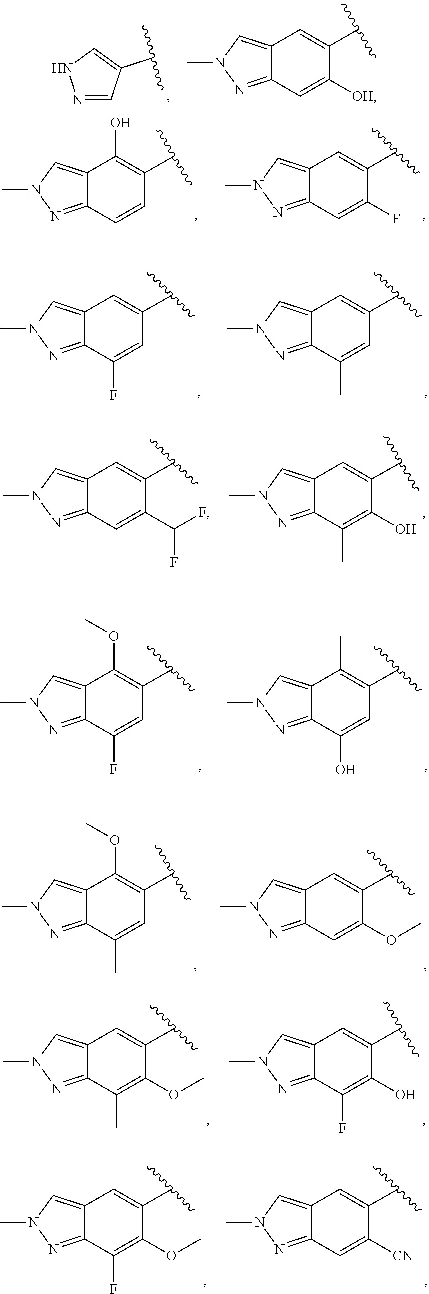

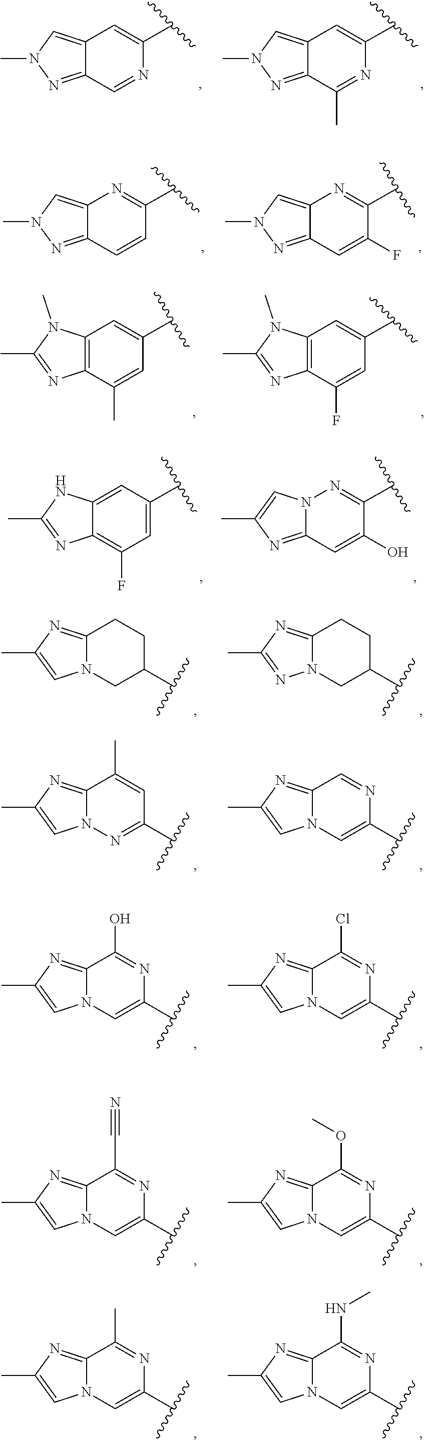

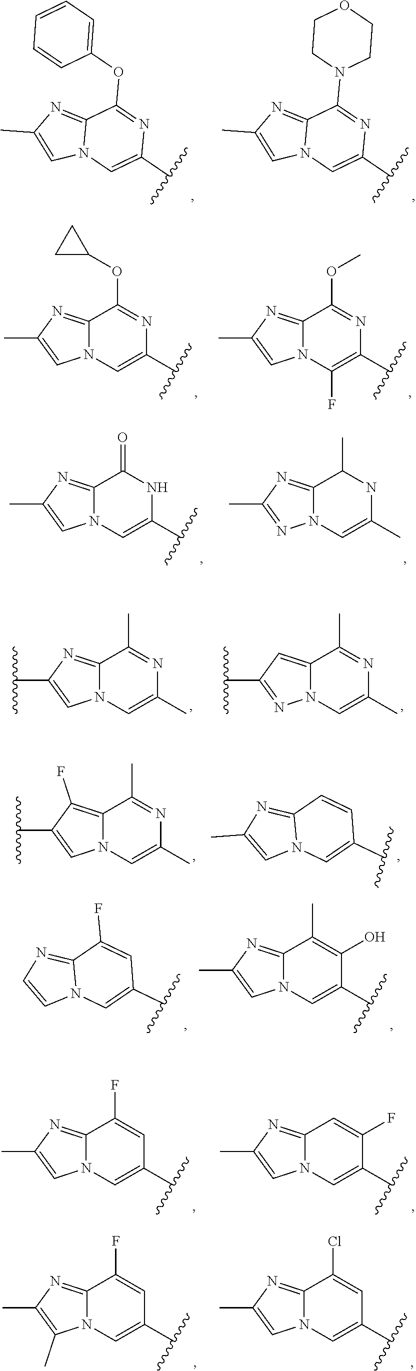

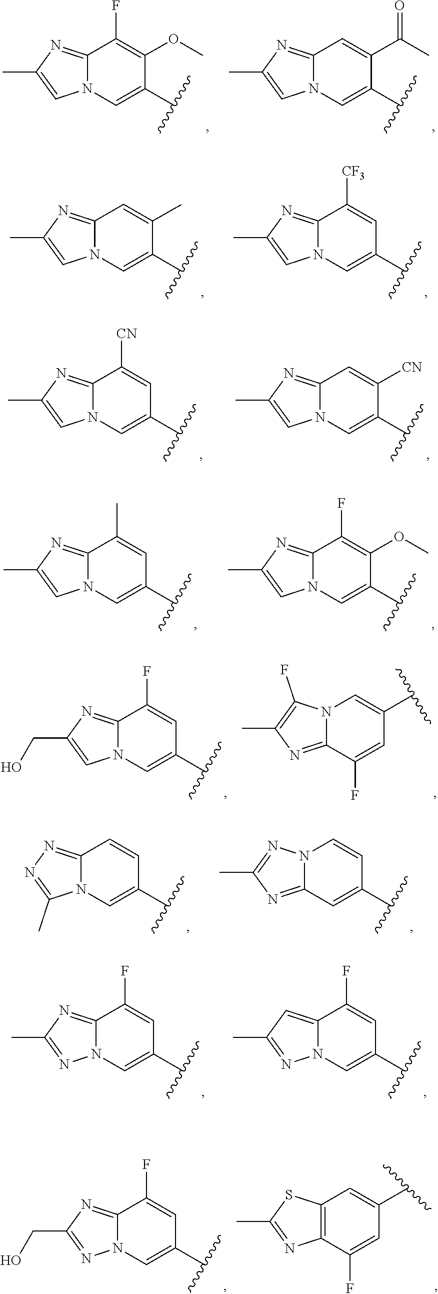

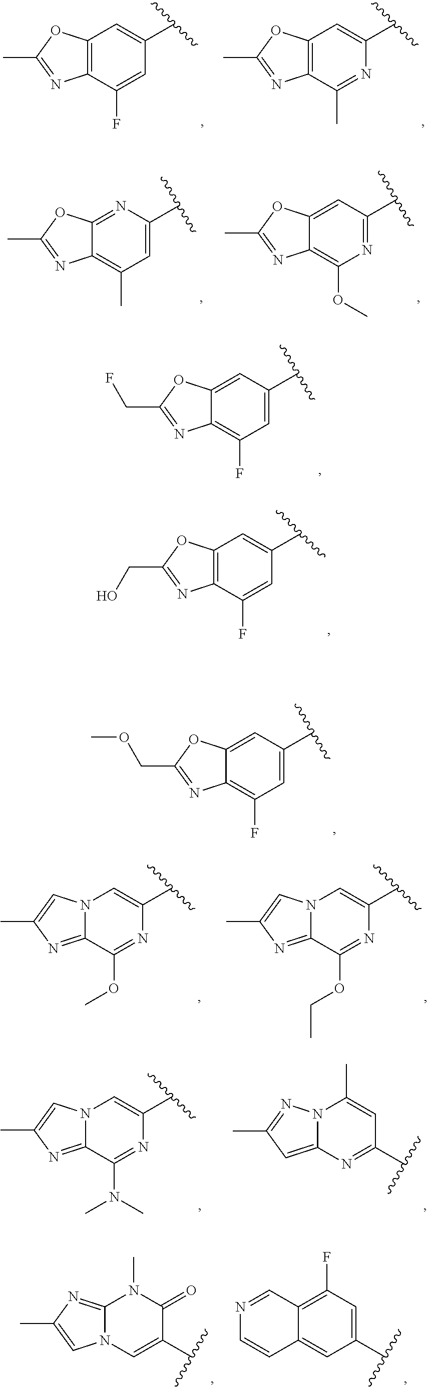

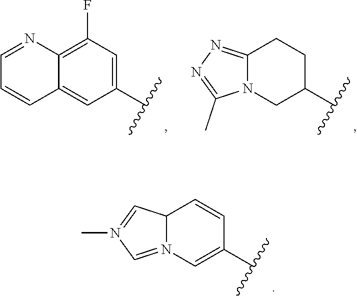

19. The compound of any one of the preceding claims, wherein B is selected from

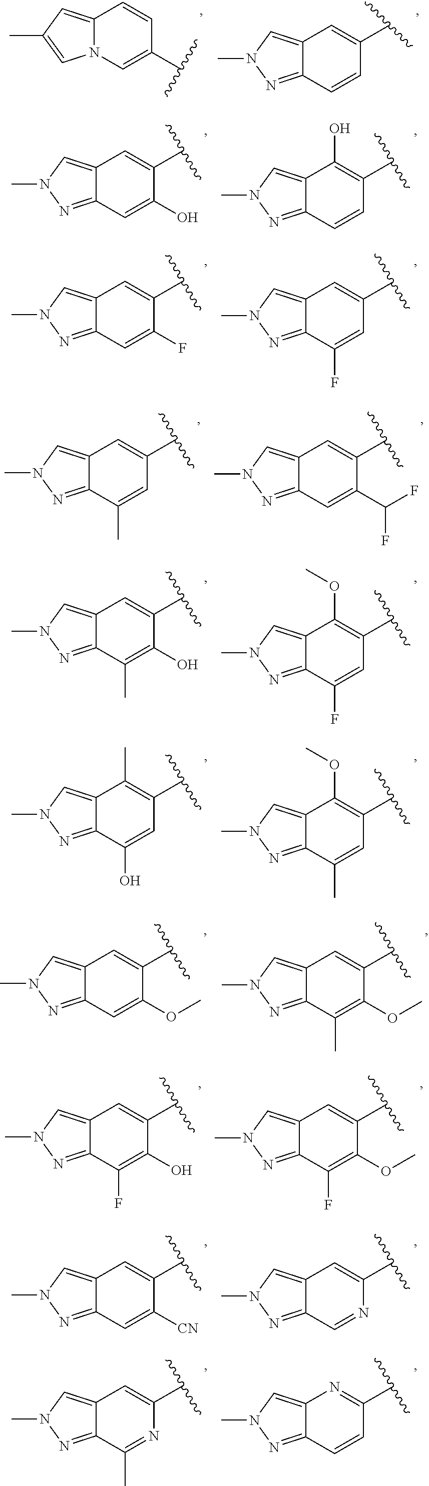

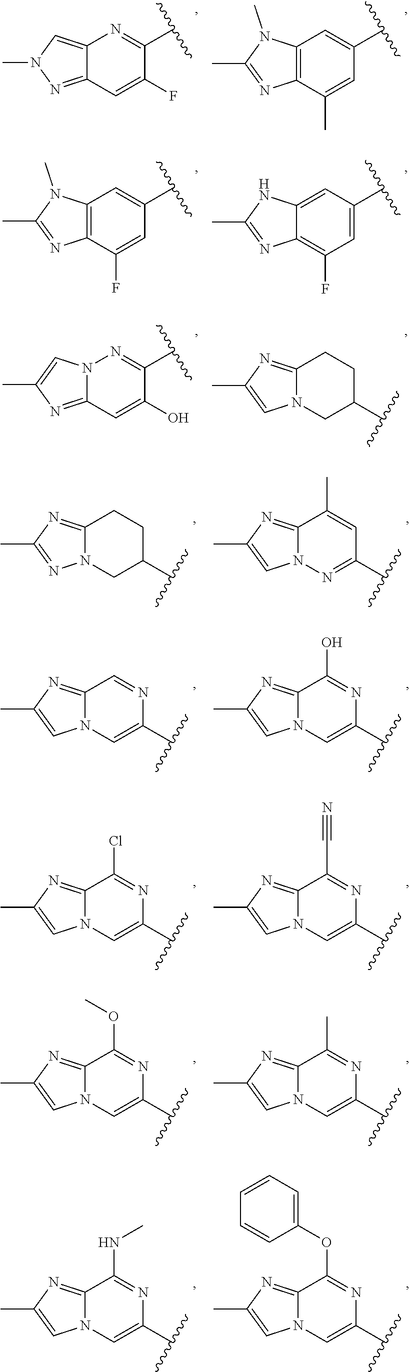

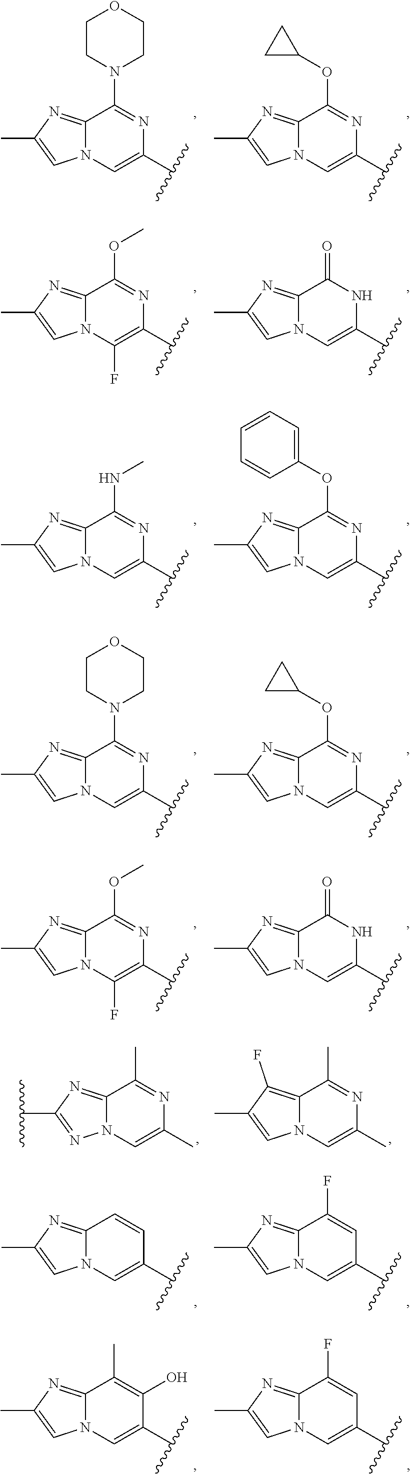

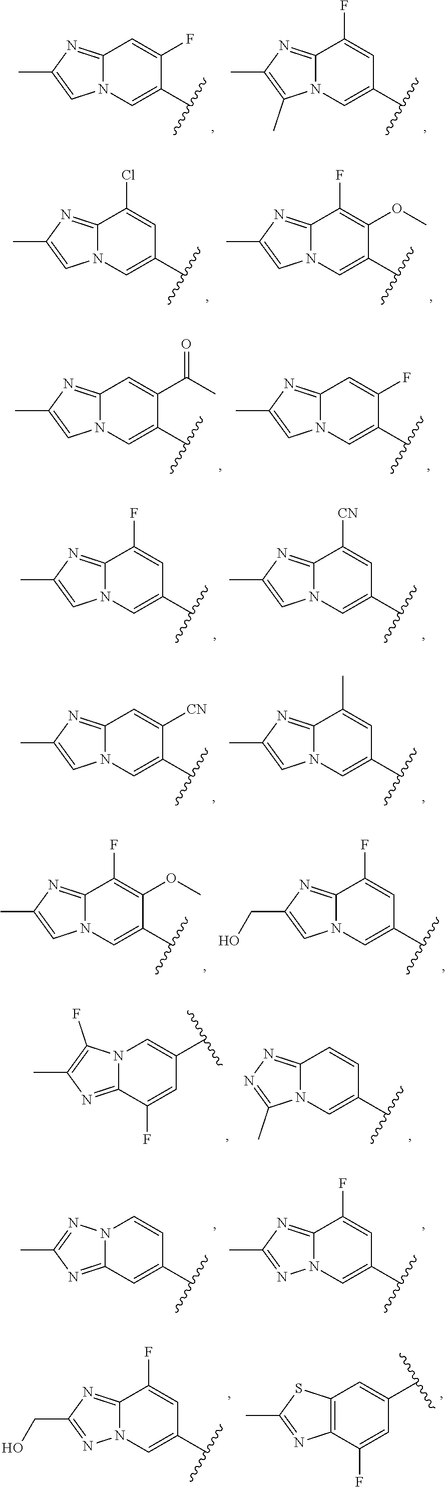

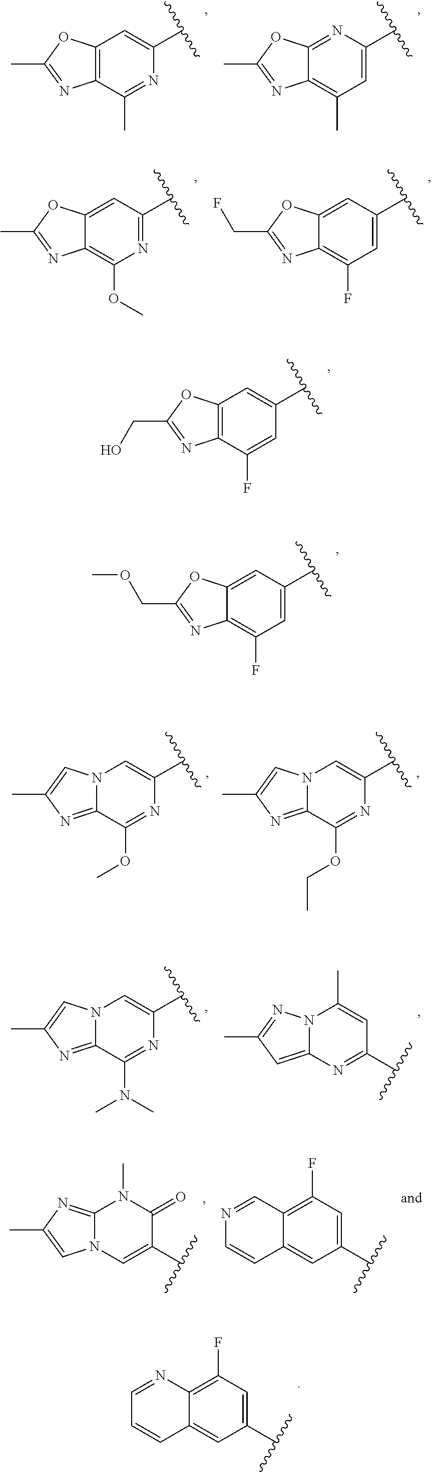

20. The compound of any one of the preceding claims, wherein A selected from

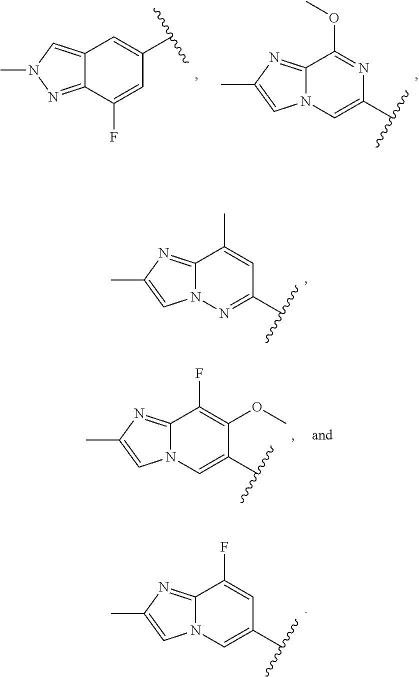

21. The compound of any one of the preceding claims, wherein A is

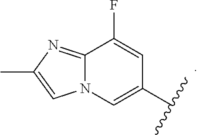 [SMILES: *c1cc(F)c2nc(C)cn2c1]

22. The compound of any one of the preceding claims, wherein one of A and B is independently a monocyclic heterocyclyl or bicyclic heterocyclyl, each of which is optionally substituted with one or more R¹.

23. The compound of any one of the preceding claims, wherein one of A and B is independently a nitrogen-containing heterocyclyl optionally substituted with one or more R¹.

24. The compound of any one of the preceding claims, wherein one of A and B is independently a 4-8 membered heterocyclyl optionally substituted with one or more R¹.

25. The compound of any one of the preceding claims, wherein one of A and B is independently selected from 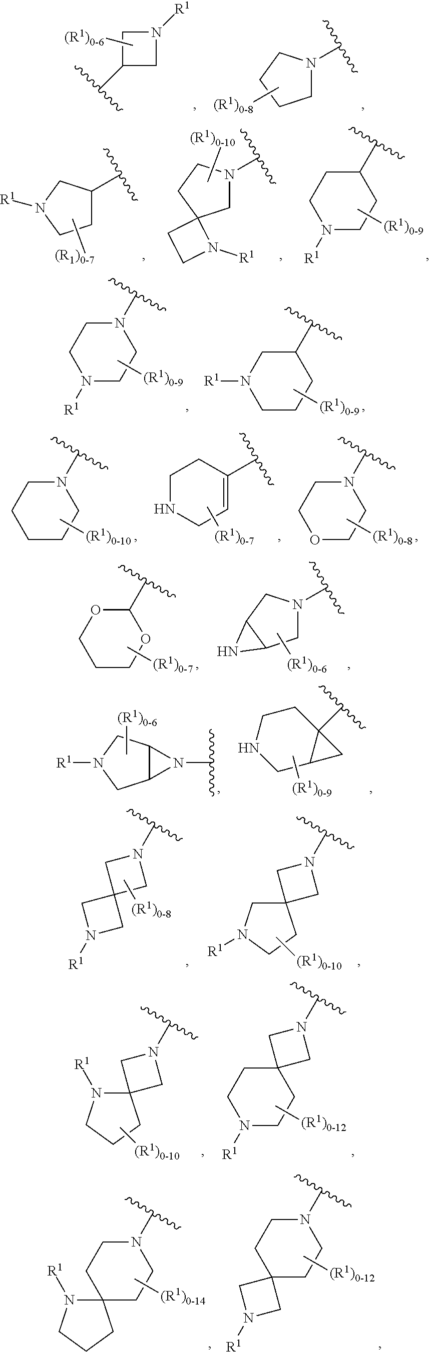 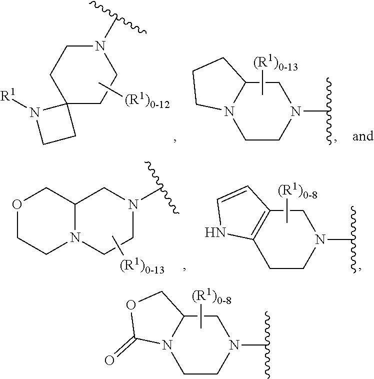
wherein R¹ is as described in claim 1.

26. The compound of any one of the preceding claims, wherein one of A and B is independently selected from 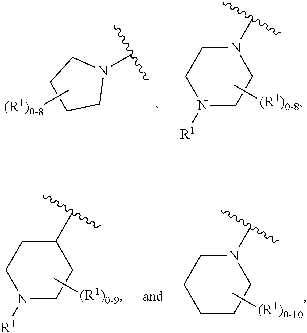
wherein R¹ is as described in claim 1.

27. The compound of any one of the preceding claims, wherein one of A and B is 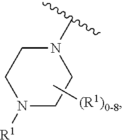
wherein R¹ is as described in claim 1.

28. The compound of any one of the preceding claims, wherein A is selected from 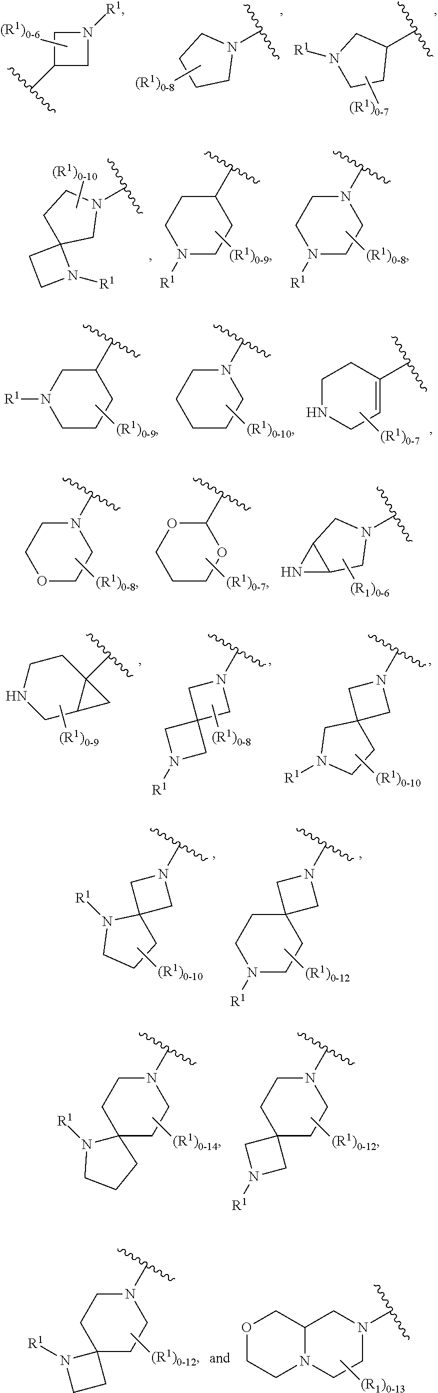
wherein R¹ is as described in claim 1.

29. The compound of any one of the preceding claims, wherein B is selected from 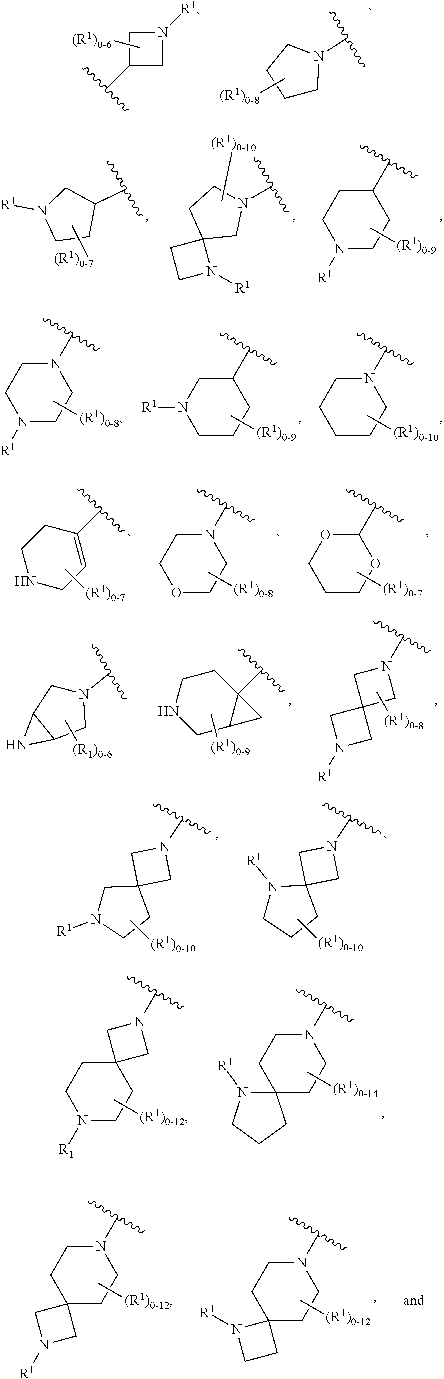 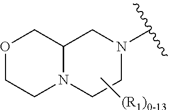
wherein R¹ is as described in claim 1.

30. The compound of any one of the preceding claims, wherein B is selected from 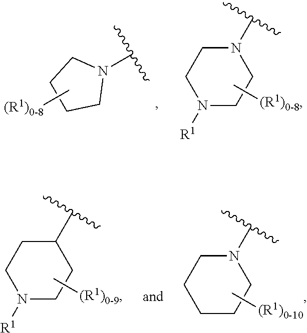
wherein R¹ is as described in claim 1.

31. The compound of any one of the preceding claims, wherein B is 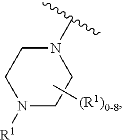
wherein R¹ is as described in claim 1.

32. The compound of any one of the preceding claims, wherein one of A and B is independently selected from

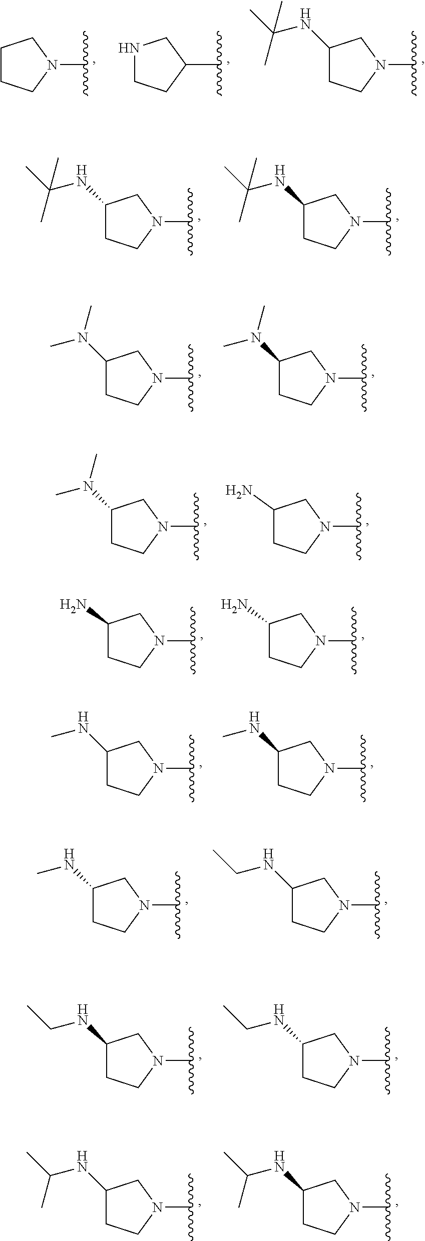

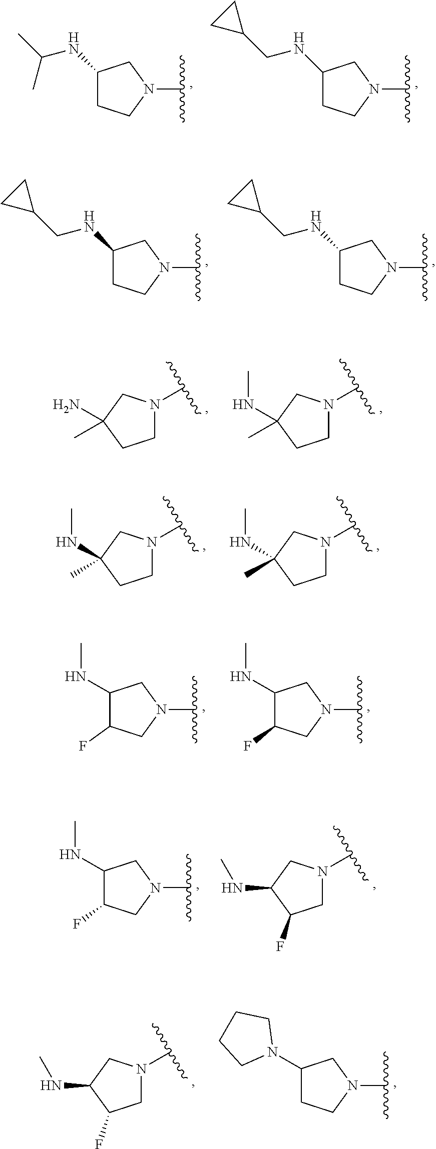

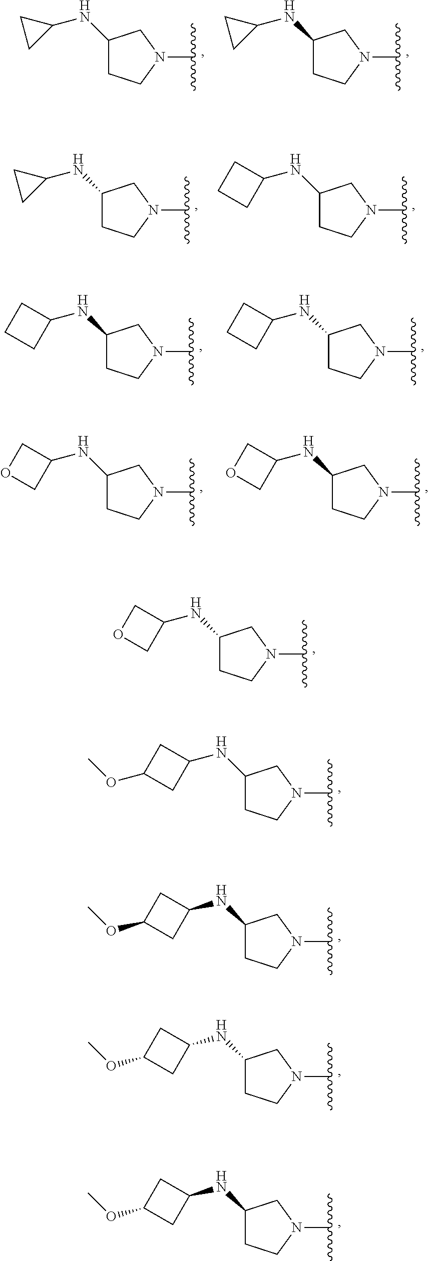

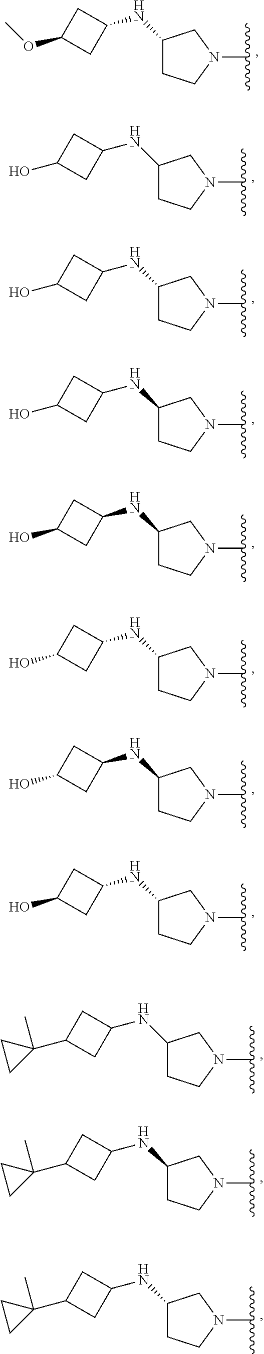

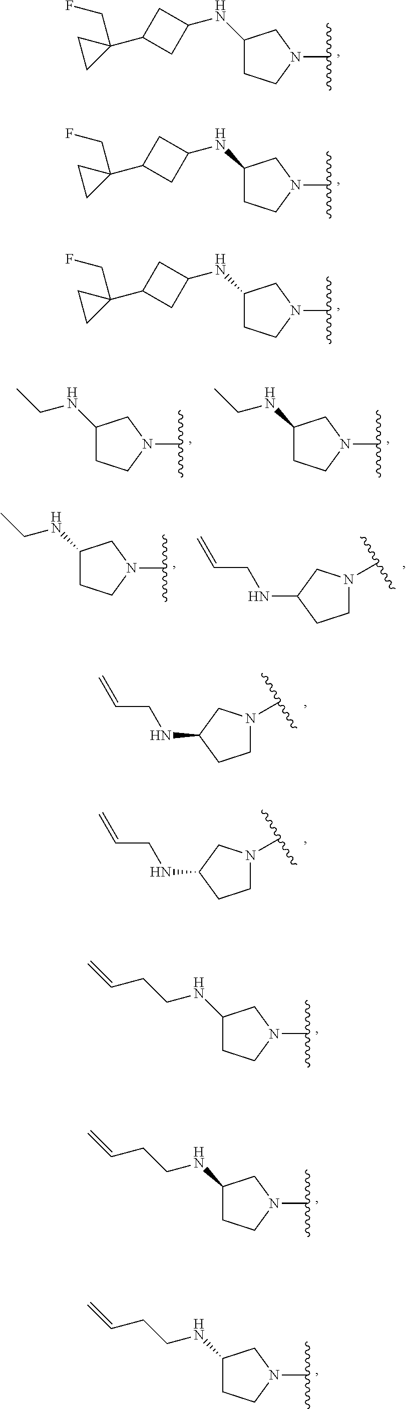

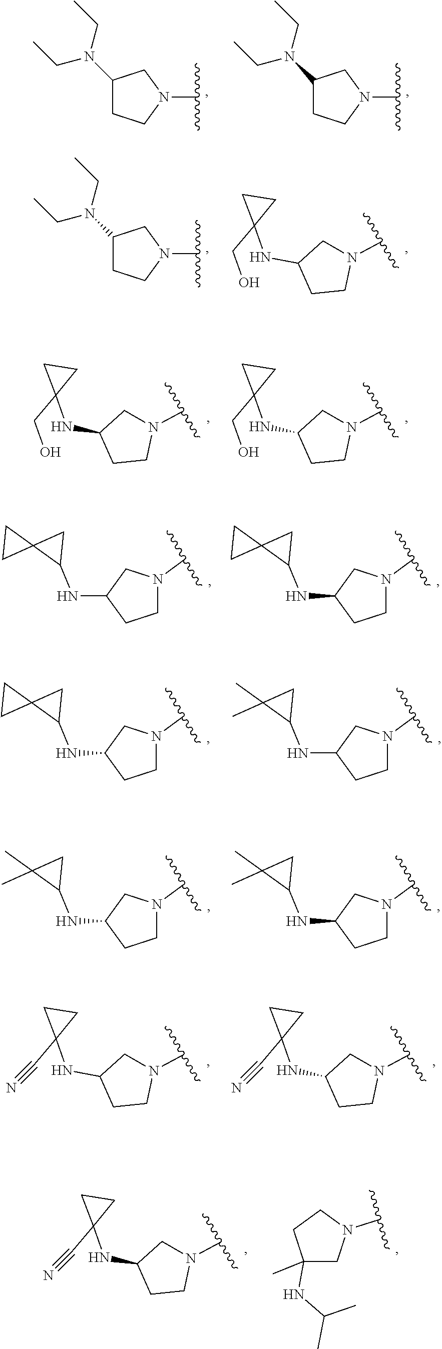

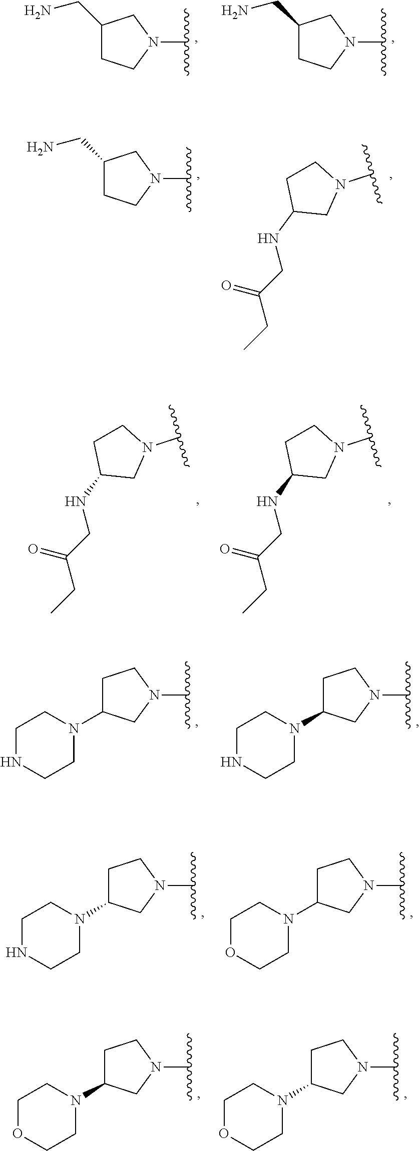

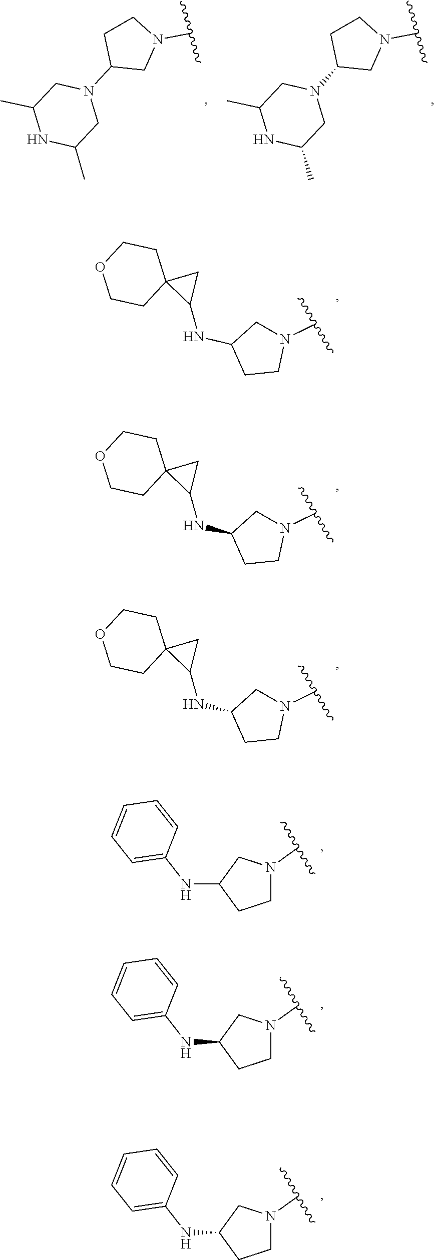

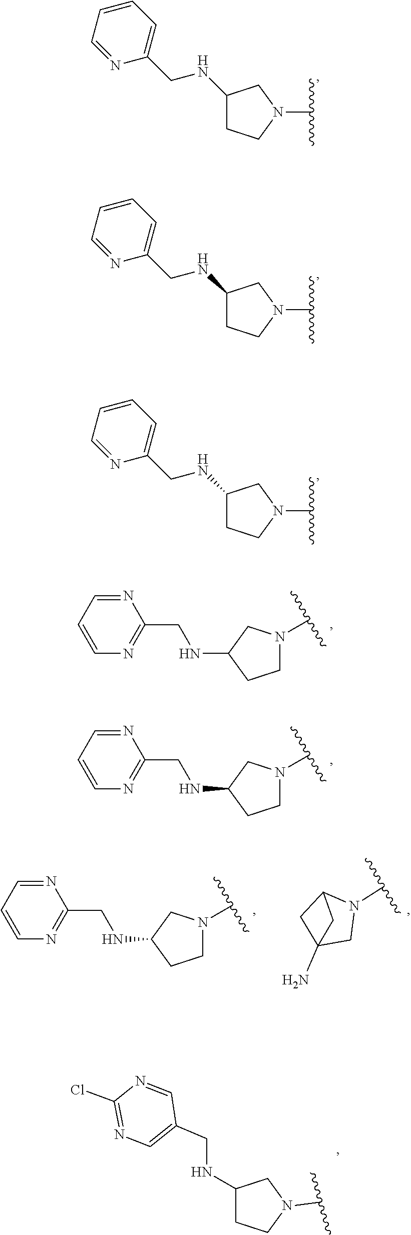

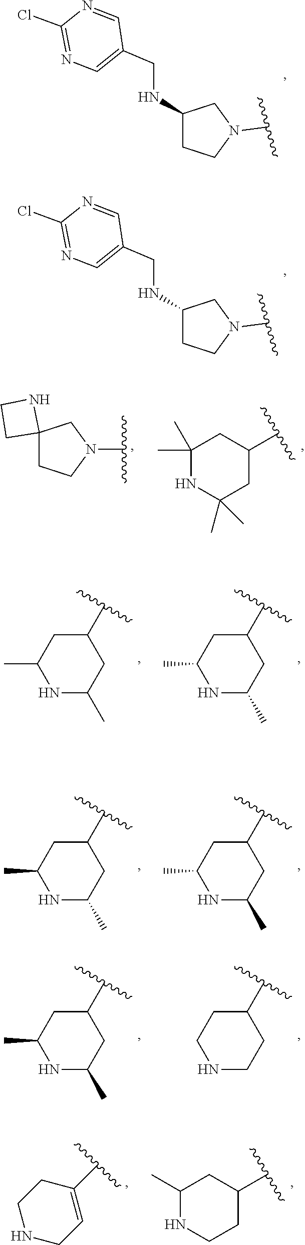

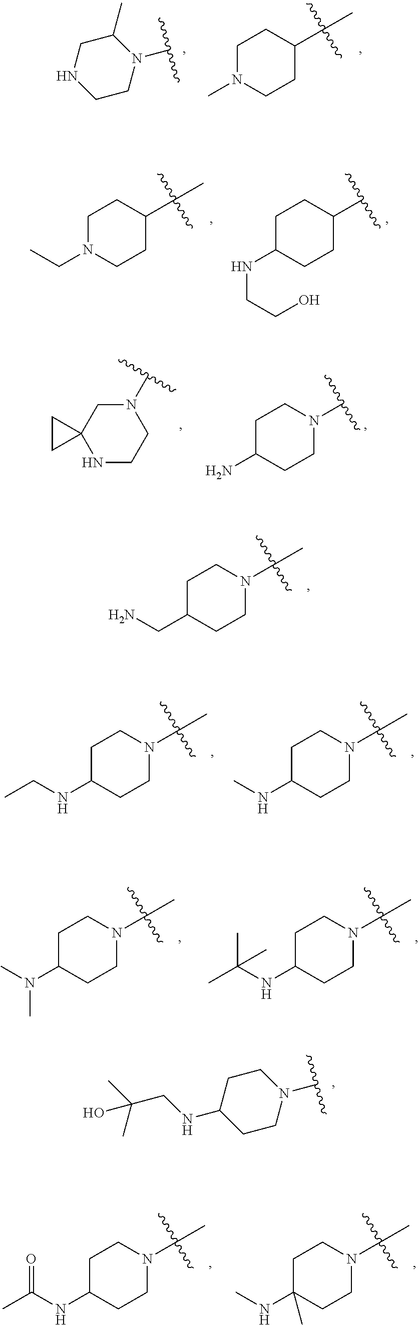

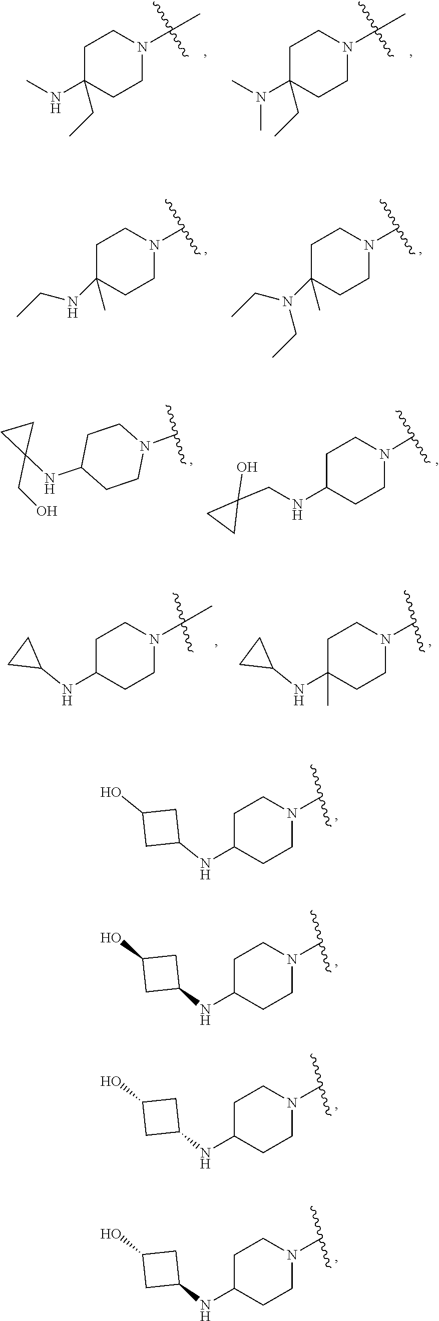

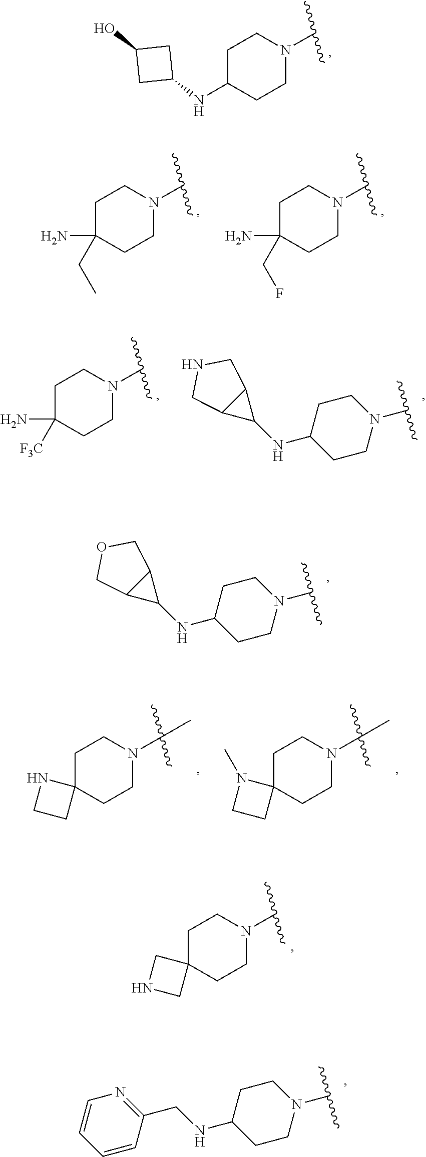

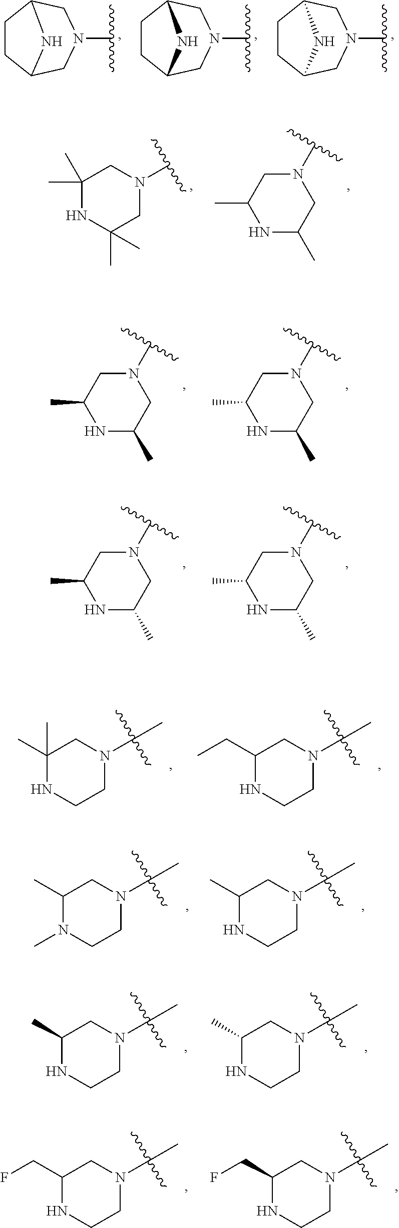

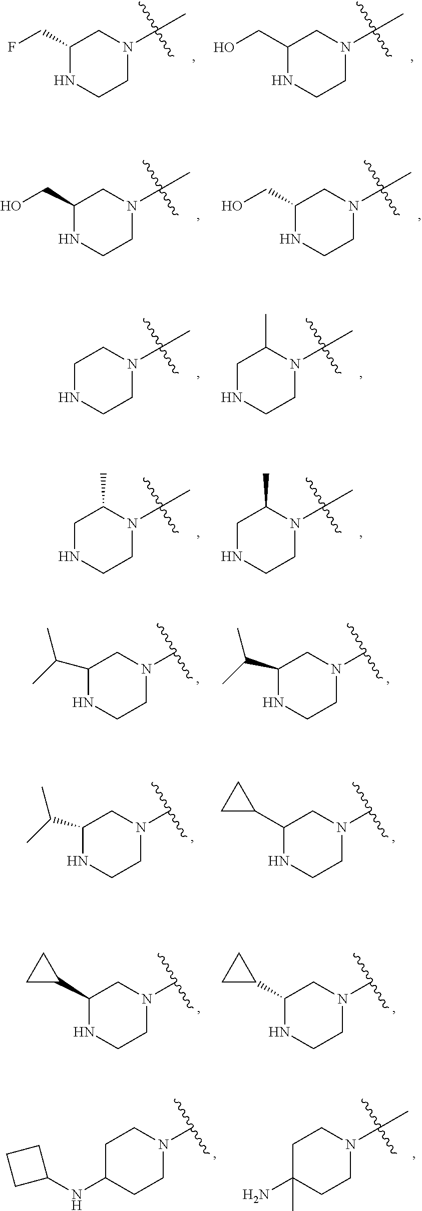

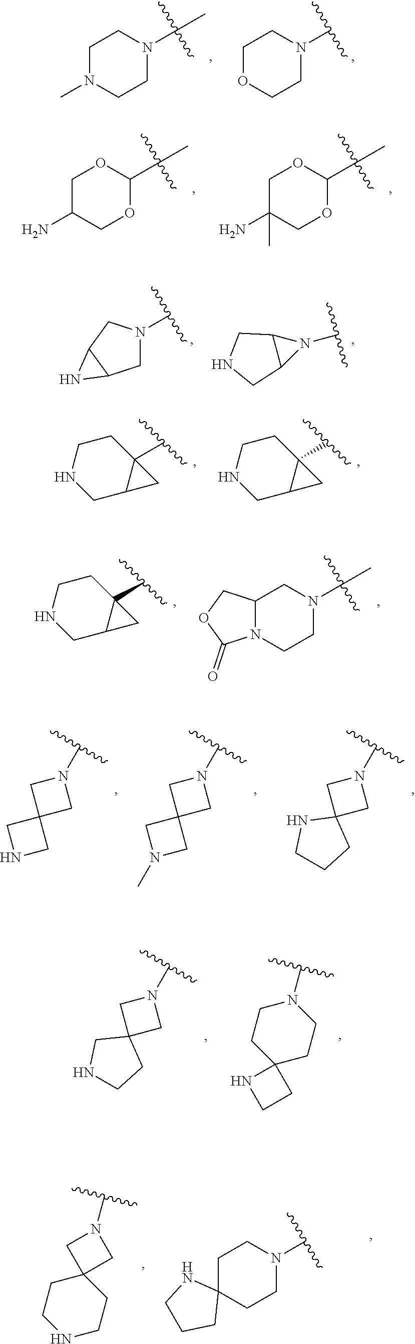

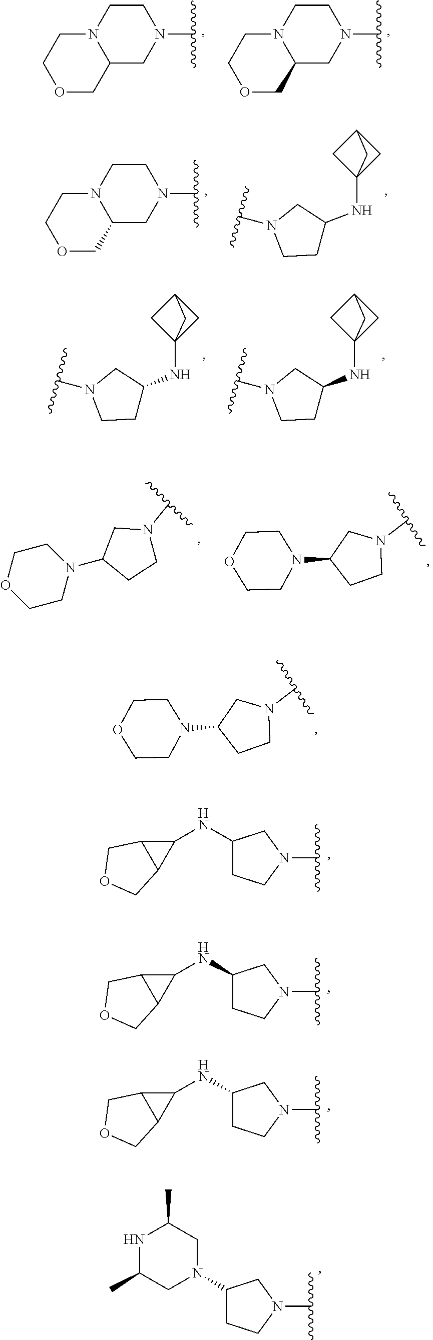

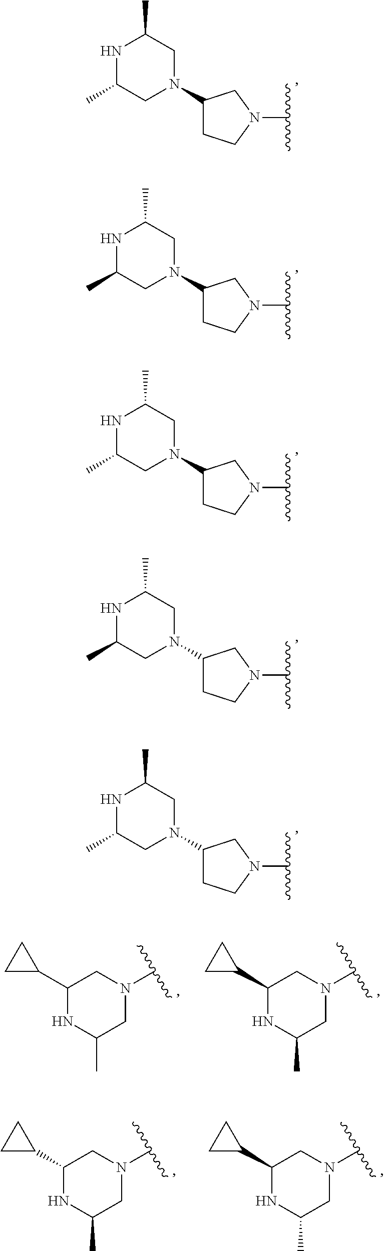

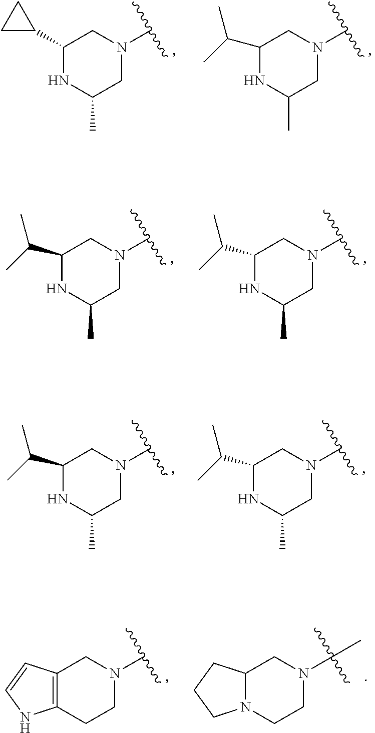

33. The compound of any one of the preceding claims, wherein one of A and B is independently selected from,

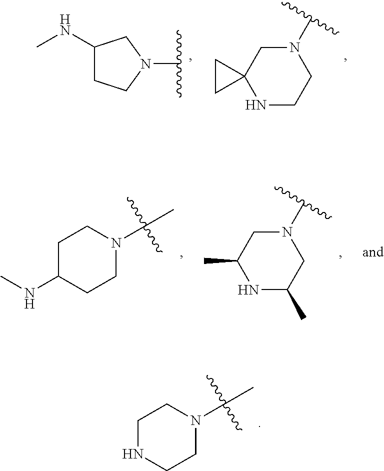

34. The compound of any one of the preceding claims, wherein one of A and B is

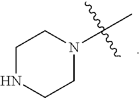 [SMILES: *N1CCNCC1]

35. The compound of any one of the preceding claims, wherein A is selected from

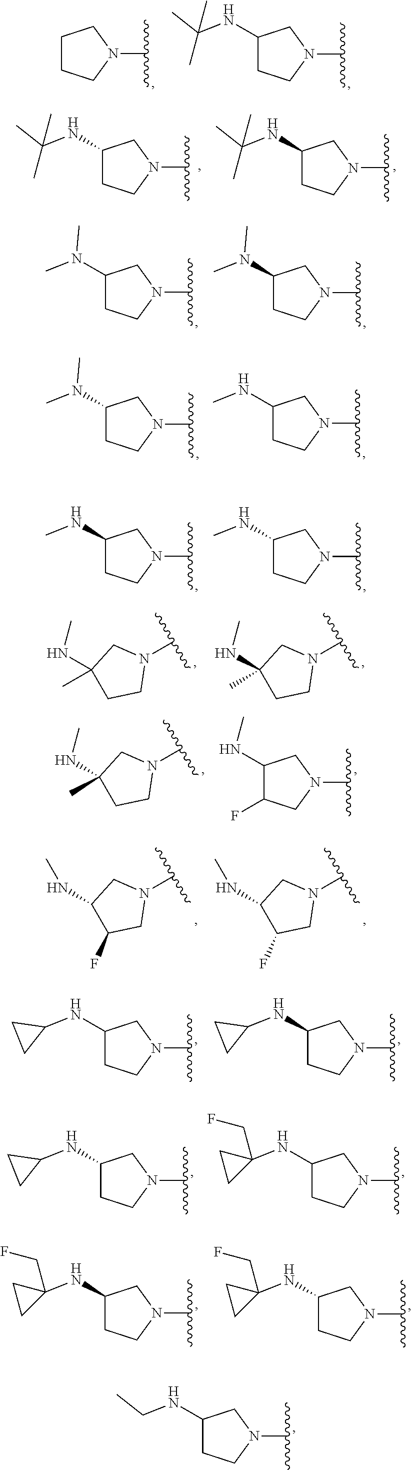

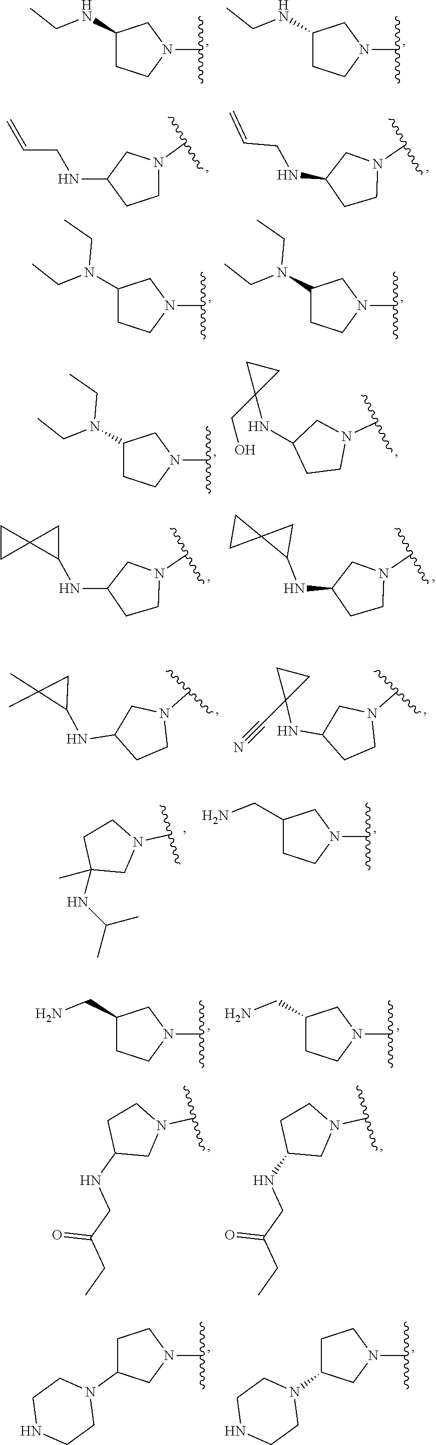

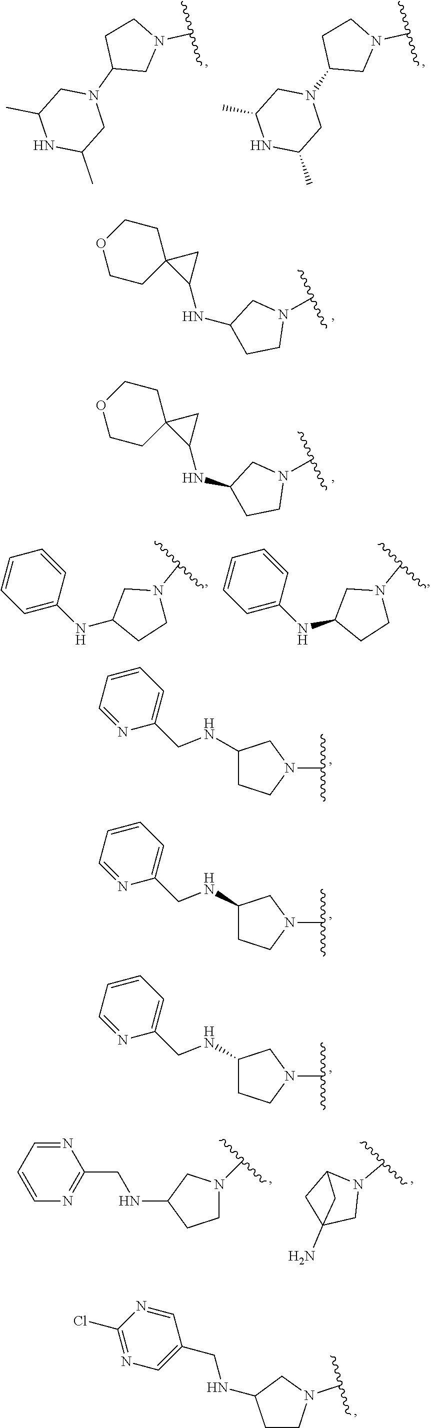

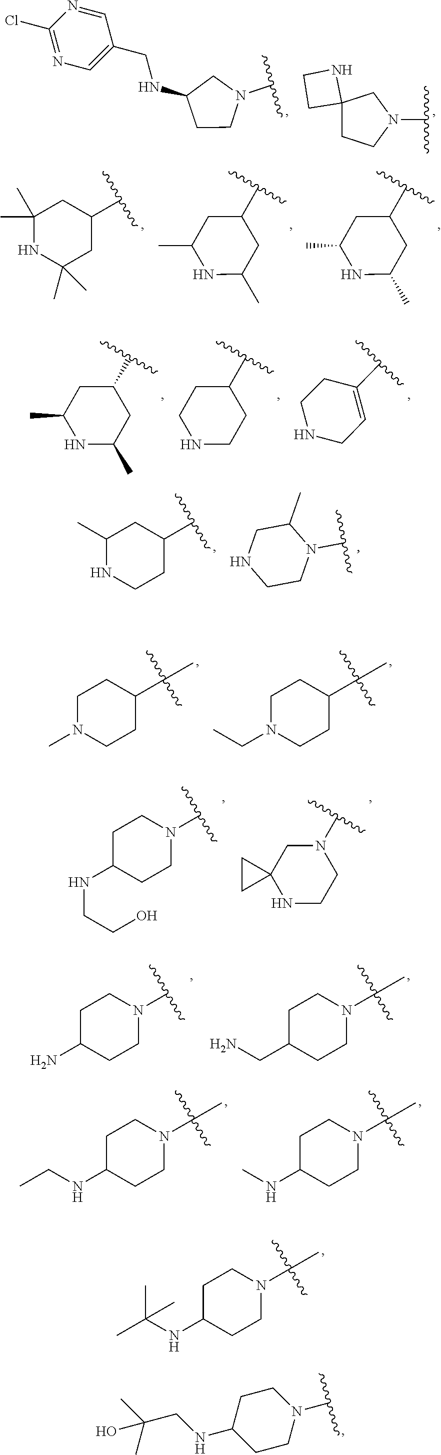

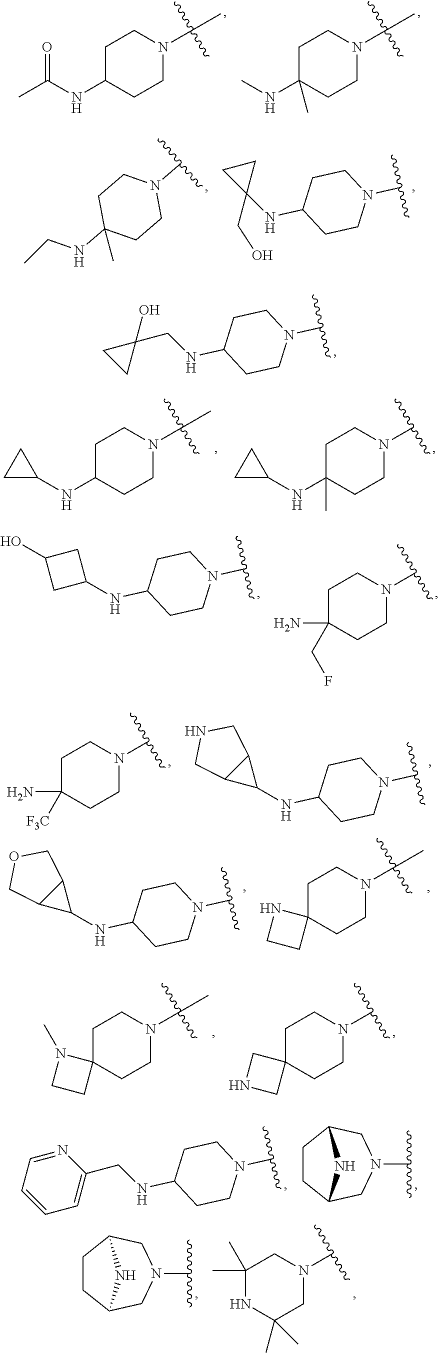

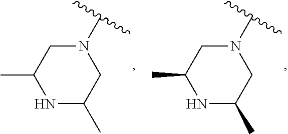

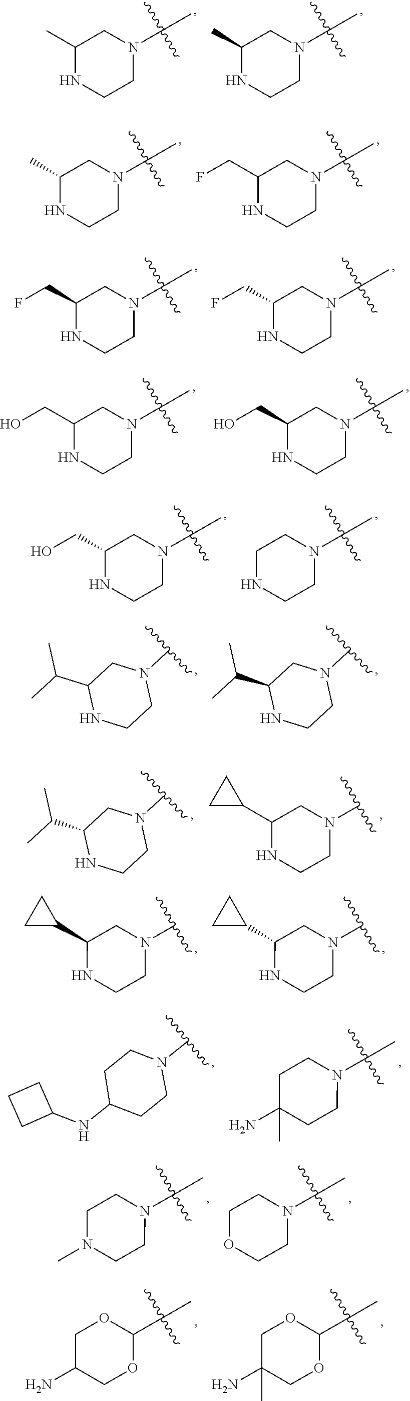

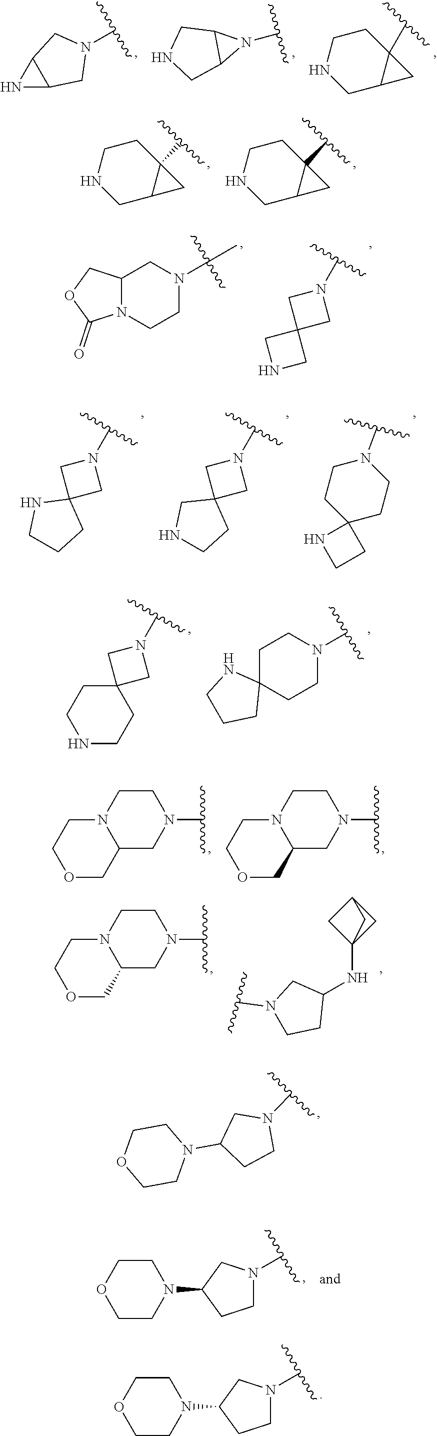

36. The compound of any one of the preceding claims wherein B is selected from

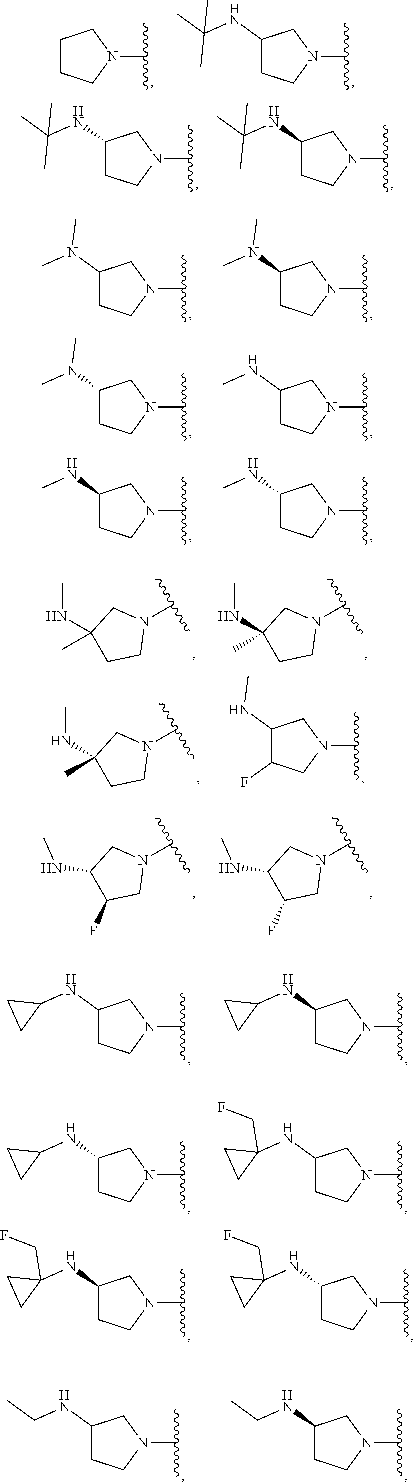

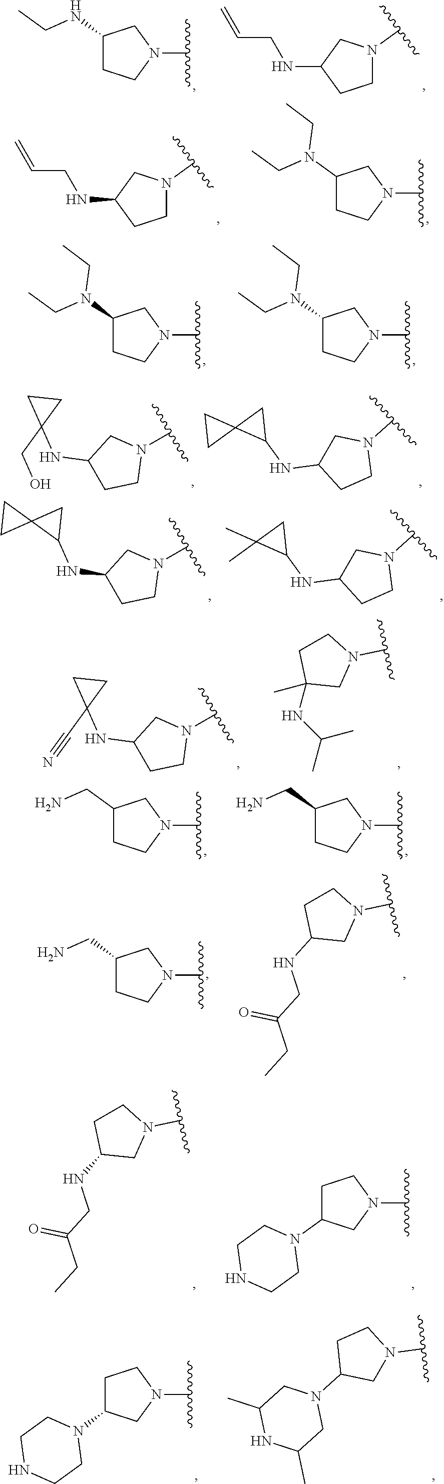

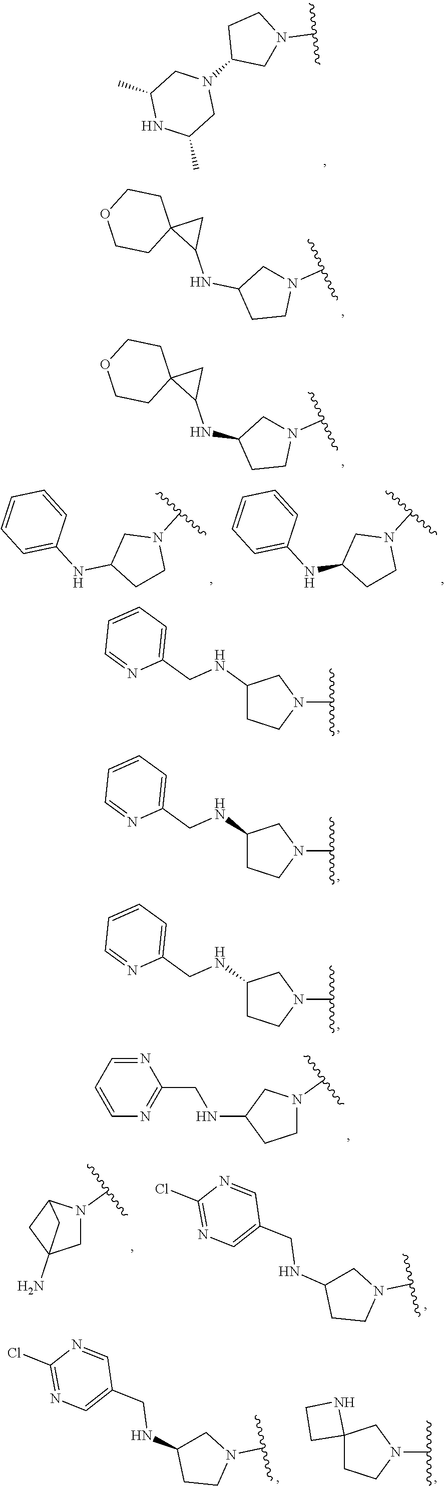

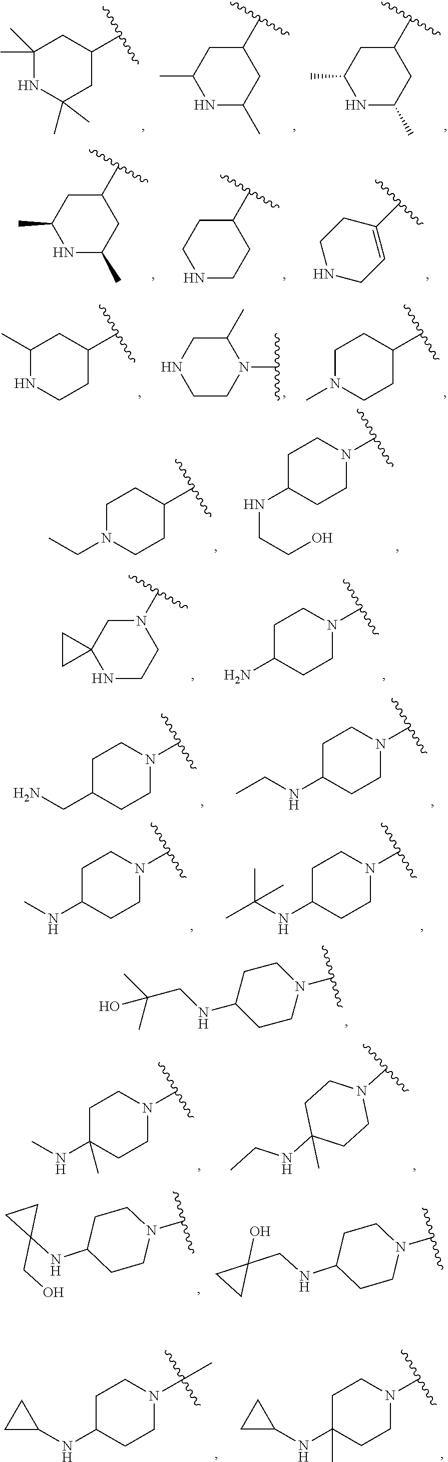

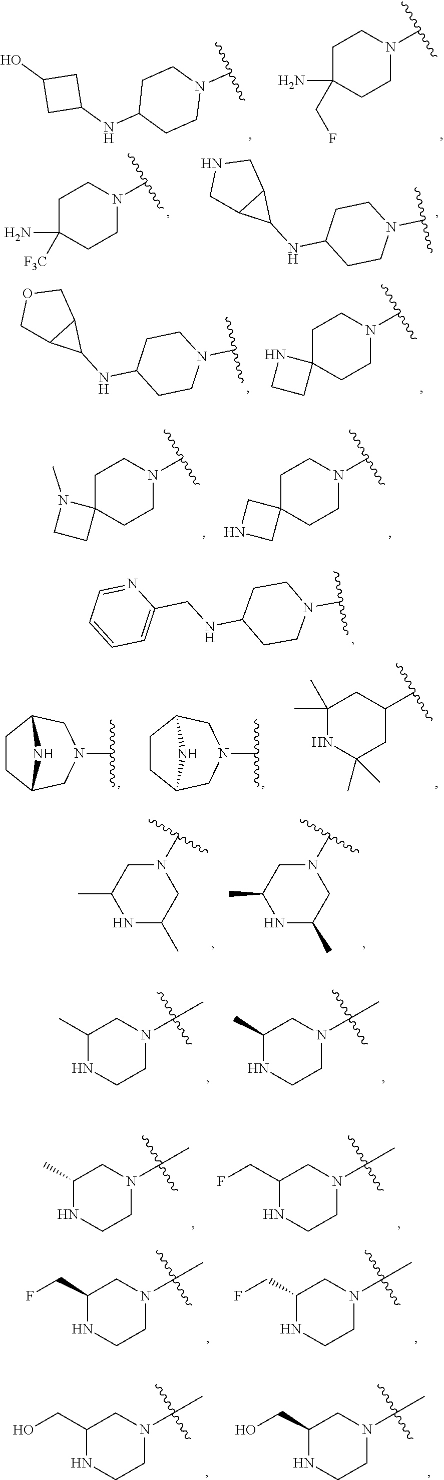

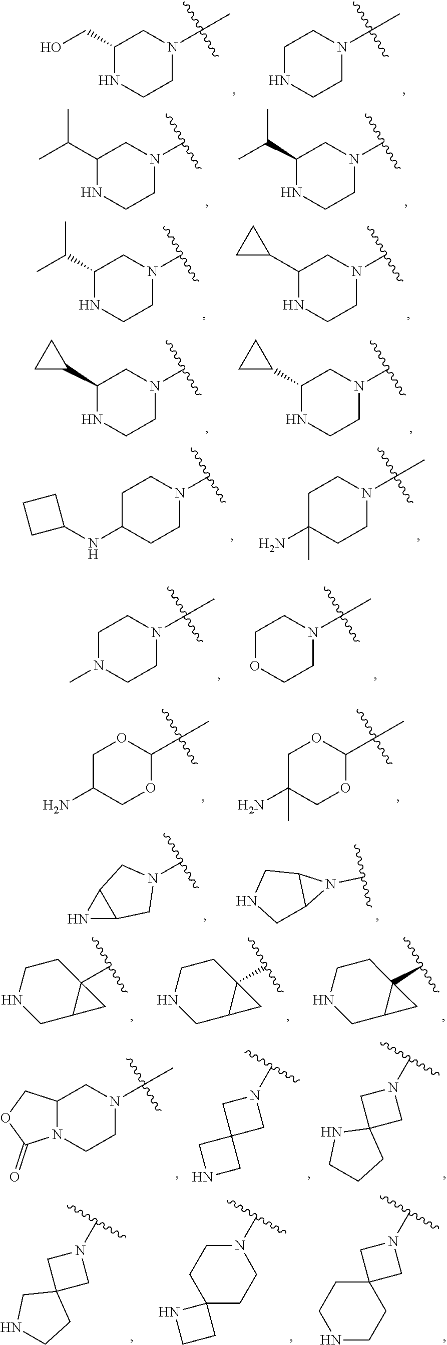

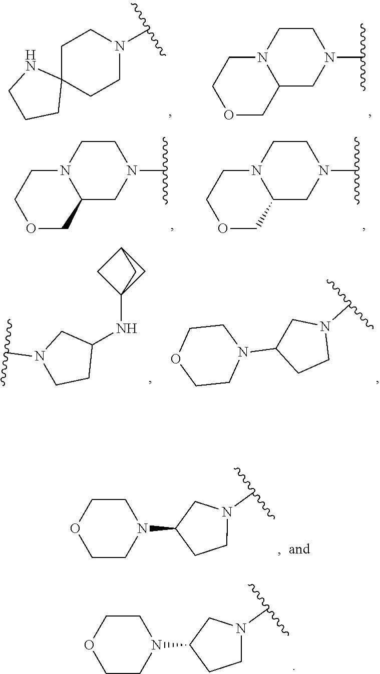

37. The compound of any one of the preceding claims, wherein B is selected from,

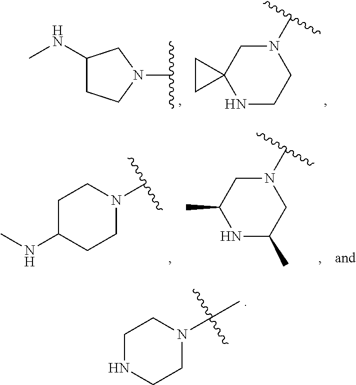

38. The compound of any one of the preceding claims, wherein B is

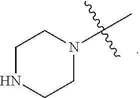 [SMILES: *N1CCNCC1]

39. The compound of any one of the preceding claims, wherein one of L¹ and L² is independently absent, —N(R⁴)C(O)—, or —C(O)N(R⁴)—.

40. The compound of any one of the preceding claims, wherein each of L¹ and L² is independently absent.

41. The compound of any one of the preceding claims, wherein L¹ is —C(O)N(R⁴)—.

42. The compound of any one of the preceding claims, wherein L² is absent.

43. The compound of any one of the preceding claims, wherein L¹ is —C(O)NH— and L² is absent.

44. The compound of any one of the preceding claims, wherein at least one of X, Y, and Z is N or N(R^{3c}).

45. The compound of any one of the preceding claims, wherein one of X, Y, and Z is C(R^{3a}) (e.g., CH), and the others of X, Y, and Z are each independently 4N or N(R^{3c}).

46. The compound of any one of the preceding claims, wherein Z and Y are each independently N or N(R^{3c}), and X is C(R^{3a}) (e.g., CH).

47. The compound of any one of the preceding claims, wherein X is C(R^{3a}) (e.g., CH), and Y and Z are each independently N or N(R^{3c}).

48. The compound of any one of the preceding claims, wherein 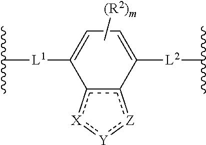
is selected from

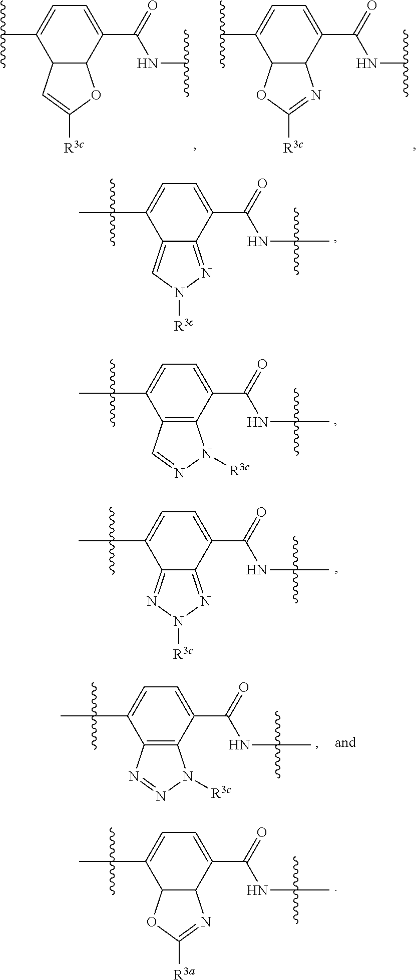

49. The compound of any one of the preceding claims, wherein 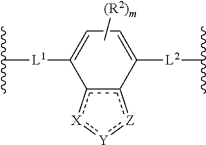
is

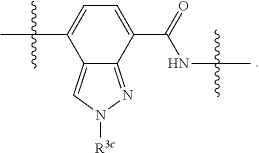

50. The compound of any one of the preceding claims, wherein R³, is C₁-C₆-alkyl, C₂-C₆-alkenyl, C₁-C₆-heteroalkyl, C₁-C₆-haloalkyl, cycloalkyl, heterocyclyl, C₁-C₆ alkylene-heterocyclyl, aryl, C₁-C₆ alkylene-aryl, heteroaryl, C₁-C₆ alkylene-heteroaryl, heteroaryl, wherein each alkyl, alkylene, alkenyl, heteroalkyl, haloalkyl, cycloalkyl, heterocyclyl, aryl, and heteroaryl is optionally substituted with one or more R′.

51. The compound of any one of the preceding claims, wherein the compound of Formula (I) is Formula (I-a): 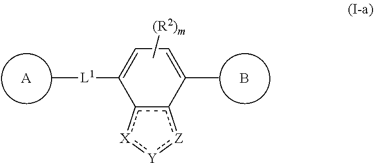
or a pharmaceutically acceptable salt, solvate, hydrate, tautomer, or stereoisomer thereof, wherein each of A, B, L¹, X, Y, Z, R², m, and subvariables thereof is as defined in claim 1.

52. The compound of any one of the preceding claims, wherein the compound of Formula (I) is Formula (I-b): 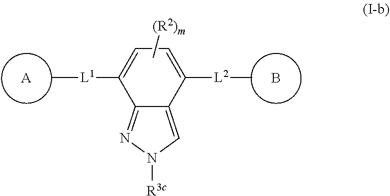
or a pharmaceutically acceptable salt, solvate, hydrate, tautomer, or stereoisomer thereof, wherein A, B, L¹, R², R^{3c}, m, and subvariables thereof are as defined in claim 1.

53. The compound of any one of the preceding claims, wherein the compound of Formula (I) is a compound of Formula (I-c): 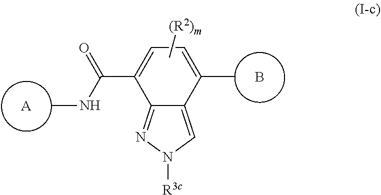
or a pharmaceutically acceptable salt, solvate, hydrate, tautomer, or stereoisomer thereof, wherein each of A, B, R², R^{3c}, m, and subvariables thereof are defined as in claim 1.

54. The compound of claim 52, wherein:
A is heteroaryl (e.g., bicyclic heteroaryl) optionally substituted with R¹;
B is heterocyclyl (e.g., monocyclic heterocyclyl), optionally substituted with one or more R¹; and
R^{3c} is C₁-C₆-alkyl, C₂-C₆-alkenyl, C₁-C₆-heteroalkyl, C₁-C₆-haloalkyl, cycloalkyl, heterocyclyl, C₁-C₆ alkylene-heterocyclyl, aryl, C₁-C₆ alkylene-aryl, heteroaryl, C₁-C₆ alkylene-heteroaryl, heteroaryl, wherein each alkyl, alkylene, alkenyl, heteroalkyl, haloalkyl, cycloalkyl, heterocyclyl, aryl, and heteroaryl is optionally substituted with one or more R⁸;
wherein each of R¹ and R⁸ are defined as in claim 1.

55. The compound of any one of the preceding claims, wherein the compound of Formula (I) is a compound of Formula (I-d): 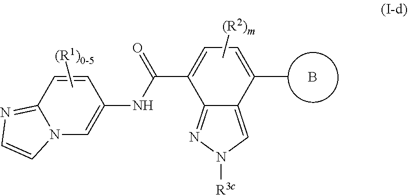
or a pharmaceutically acceptable salt, solvate, hydrate, tautomer, or stereoisomer thereof, wherein each of B, R¹, R², R^{3c}, m, and subvariables thereof are defined as in claim 1.

56. The compound of any one of the preceding claims, wherein the compound of Formula (I) is a compound of Formula (I-e): 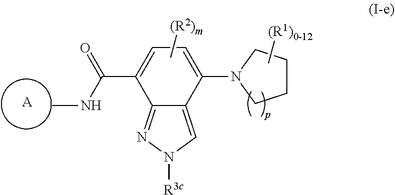
or a pharmaceutically acceptable salt, solvate, hydrate, tautomer, or stereoisomer thereof, wherein p is 0, 1, 2, or 3, and each of A, R¹, R², R^{3c}, m, and subvariables thereof are defined as in claim 1.

57. The compound of any one of the preceding claims, wherein the compound is selected from a compound listed in Table 1, or a pharmaceutically acceptable salt, solvate, hydrate, tautomer, or stereoisomer thereof.

58. A compound of Formula (II): 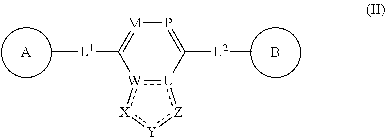
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
M and P are each independently C(R²) or N;
U and W are each independently C or N;
X, Y, and Z are each independently C(R^{3a}), N, N(R^{3c}), O, or S, wherein at least one of X, Y, and Z is N or N(R^{3c}), and the bonds in the ring comprising U, W, X, Y, and Z may be single or double bonds as valency permits;
each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶;
each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A};
R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D};
R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R′;
each R⁴ is independently hydrogen, C₁-C₆-alkyl, C₁-C₆-heteroalkyl, or C₁-C₆-haloalkyl;
each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or
R^{B} and R^{C} together with the atom to which they are attached form a 3-7-membered heterocyclyl ring optionally substituted with one or more R⁷;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁷ is C₁-C₆-alkyl, halo, cyano, oxo, or —OR^{A1};
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A1} is hydrogen or C₁-C₆-alkyl; and
x is 0, 1, or 2.

59. The compound of claim 58, wherein each of A and B is independently heteroaryl or heterocyclyl, each of which is optionally substituted with one or more R¹.

60. The compound of any one of the preceding claims, wherein one of A and B is independently a monocyclic heteroaryl or bicyclic heteroaryl, each of which is optionally substituted with one or more R¹.

61. The compound of any one of the preceding claims, wherein one of A and B is independently a bicyclic heteroaryl optionally substituted with one or more R¹.

62. The compound of any one of the preceding claims, wherein one of A and B is independently a nitrogen-containing heteroaryl optionally substituted with one or more R¹.

63. The compound of any one of the preceding claims, wherein one of A and B is a 5-10 membered heteroaryl optionally substituted with one or more R¹.

64. The compound of any one of the preceding claims, wherein one of A and B is independently selected from 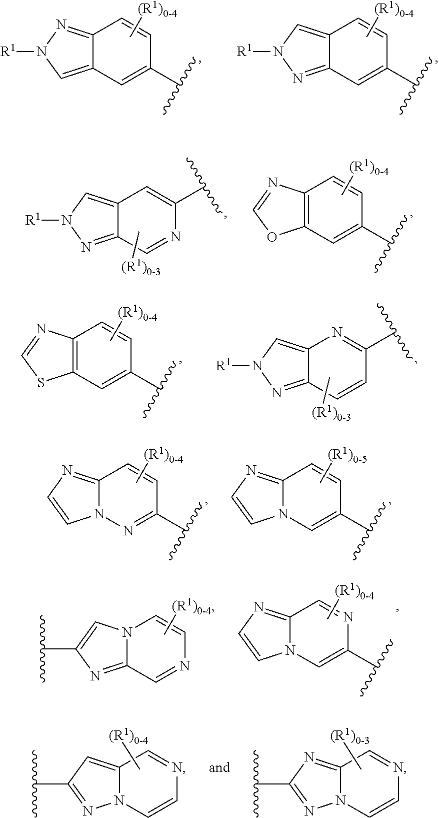
wherein R¹ is as described in claim 58.

65. The compound of any one of the preceding claims, wherein one of A and B is independently selected from 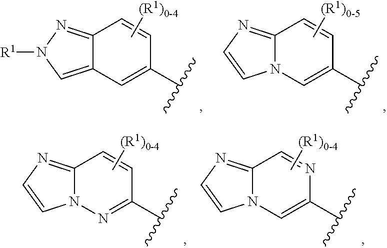
wherein R¹ is as described in claim 58.

66. The compound of any one of the preceding claims, wherein one of A and B is independently selected from 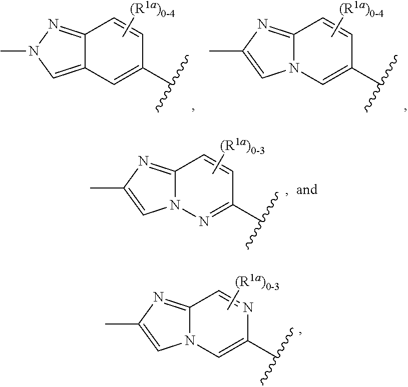
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

67. The compound of any one of the preceding claims, wherein one of A and B is independently 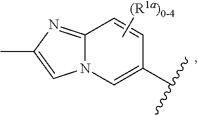
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

68. The compound of any one of the preceding claims, wherein A is selected from 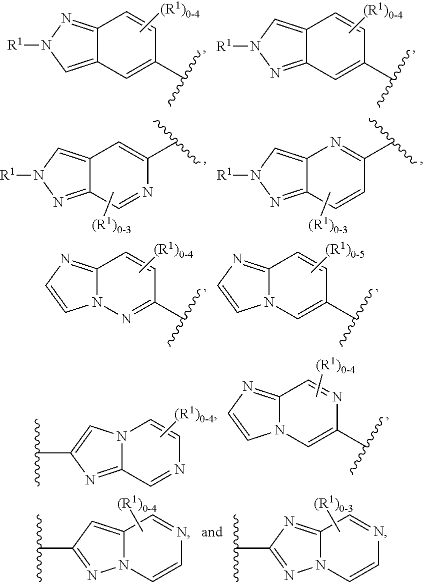
wherein R¹ is as described in claim 1.

69. The compound of any one of the preceding claims, wherein B is selected from 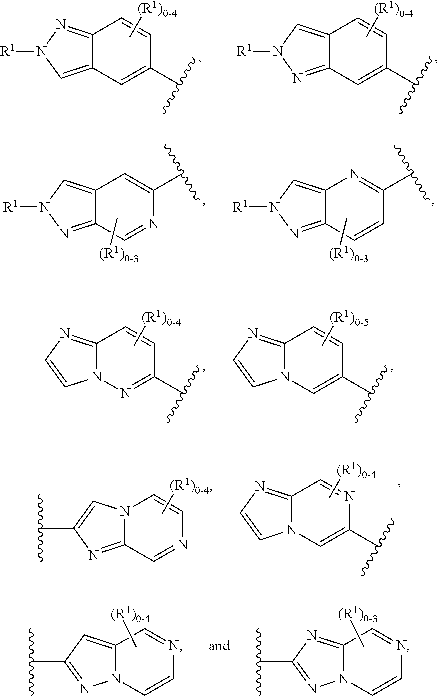
wherein R¹ is as described in claim 58.

70. The compound of any one of the preceding claims, wherein A is selected from 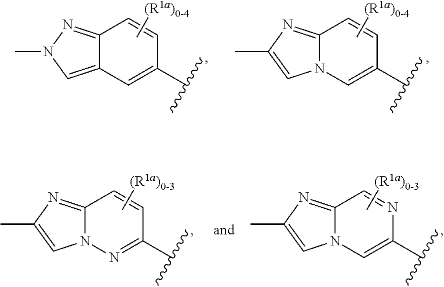
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

71. The compound of any one of the preceding claims, wherein A is 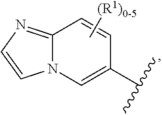
wherein R¹ is as described in claim 58.

72. The compound of any one of the preceding claims, wherein one of A and B is independently selected from

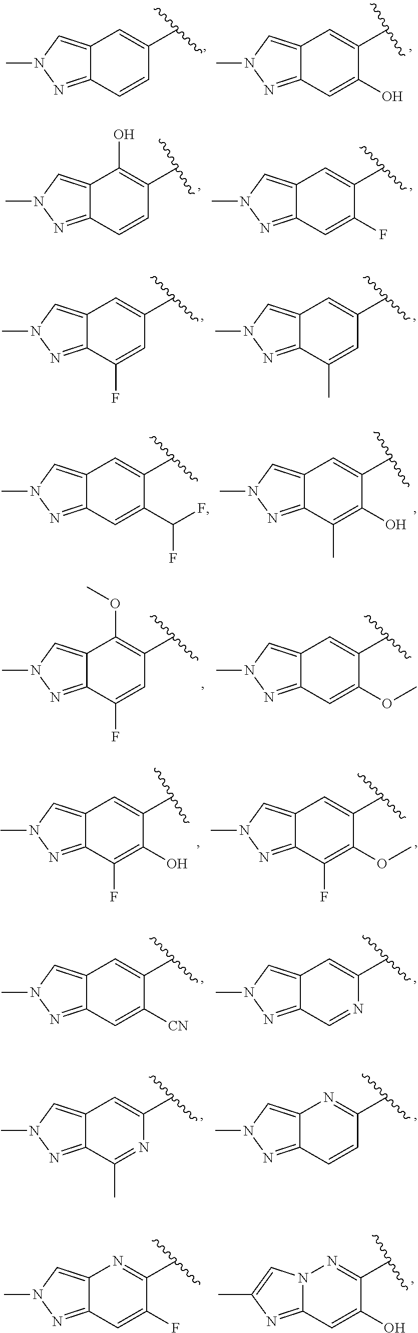

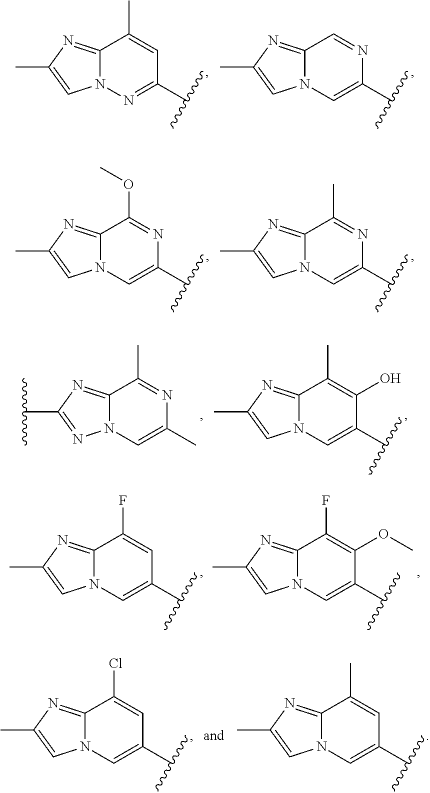

73. The compound of any one of the preceding claims, wherein one of A and B is independently selected from

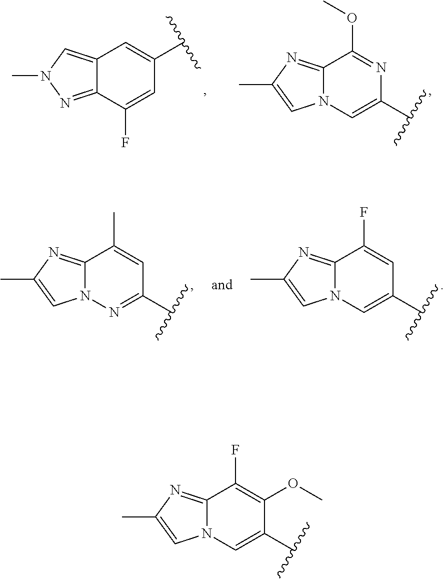

74. The compound of any one of the preceding claims, wherein one of A and B is independently

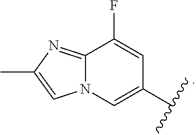 [SMILES: *c1cc(F)c2nc(C)cn2c1]

75. The compound of any one of the preceding claims, wherein A is selected from

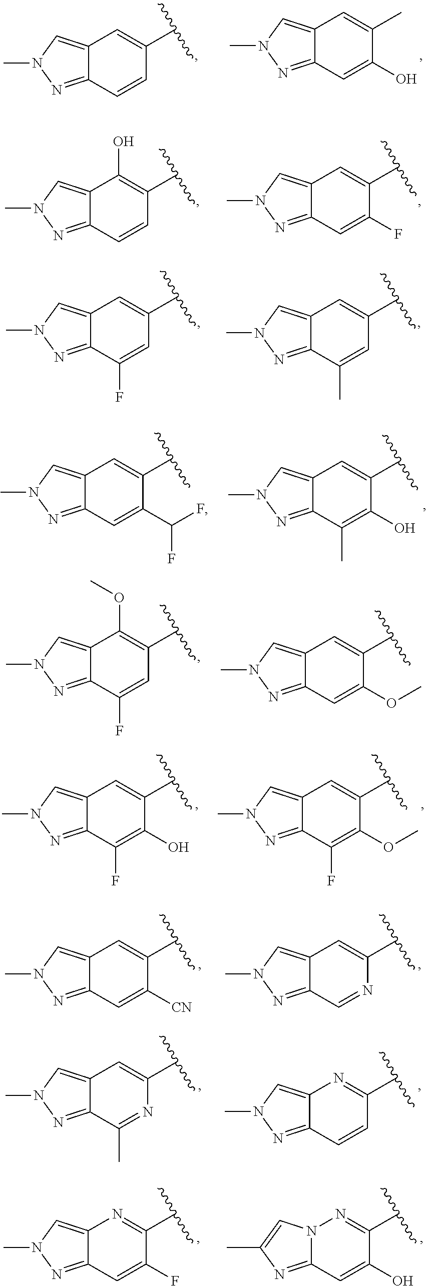

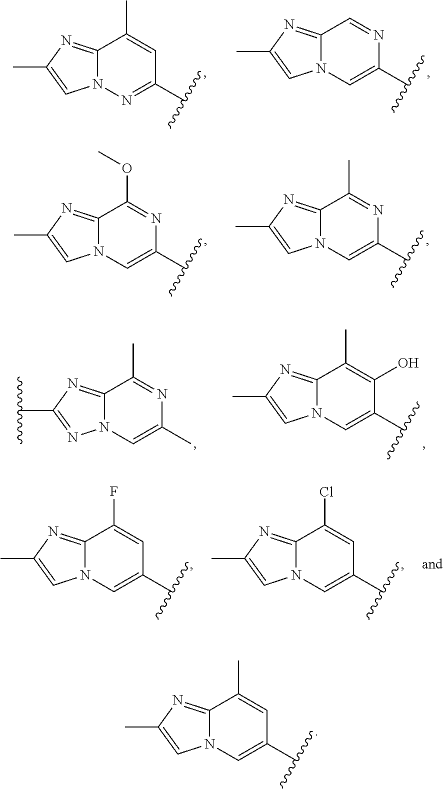

76. The compound of any one of the preceding claims, wherein B is selected from

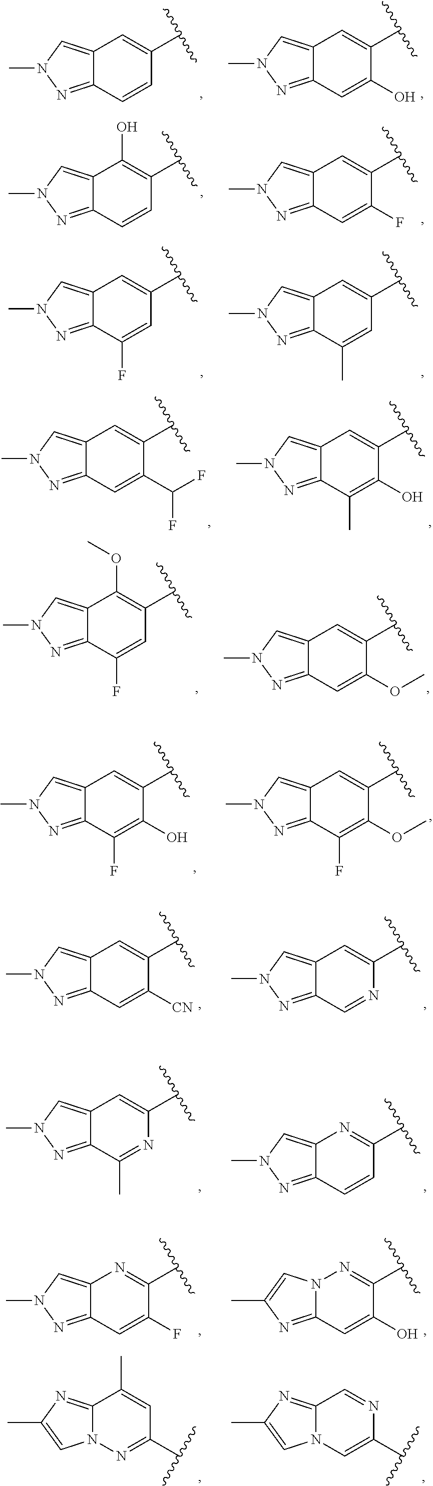

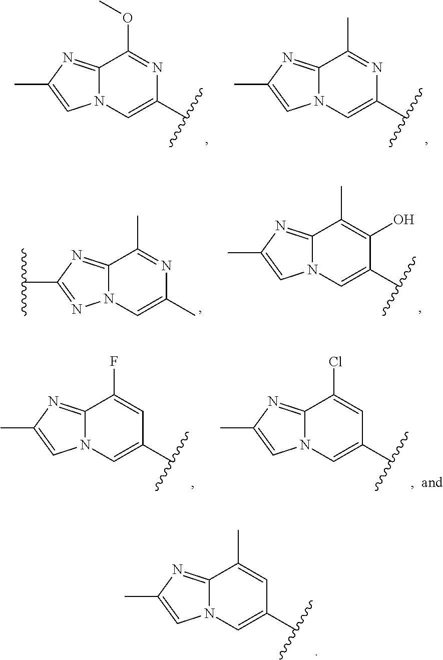

77. The compound of any one of the preceding claims, wherein A selected from

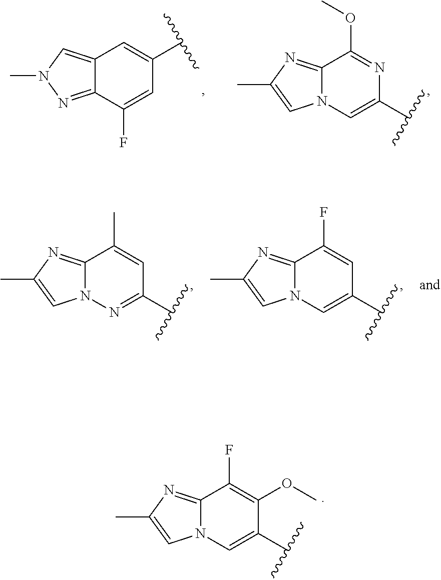

78. The compound of any one of the preceding claims, wherein A is

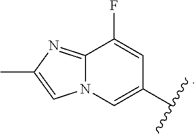 [SMILES: *c1cc(F)c2nc(C)cn2c1]

79. The compound of any one of the preceding claims, wherein one of A and B is independently a monocyclic heterocyclyl or bicyclic heterocyclyl, each of which is optionally substituted with one or more R¹.

80. The compound of any one of the preceding claims, wherein one of A and B is independently a nitrogen-containing heterocyclyl optionally substituted with one or more R¹.

81. The compound of any one of the preceding claims, wherein one of A and B is independently a 4-8 membered heterocyclyl optionally substituted with one or more R¹.

82. The compound of any one of the preceding claims, wherein one of A and B is independently selected from 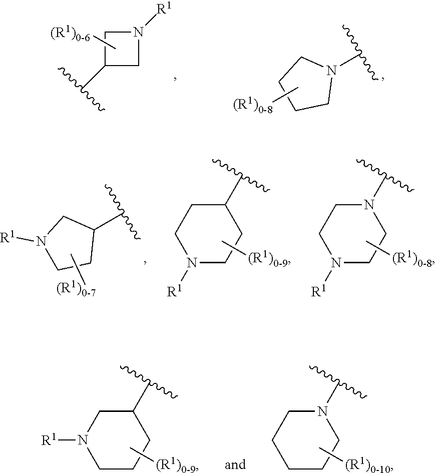
wherein R¹ is as described in claim 58.

83. The compound of any one of the preceding claims, wherein one of A and B is independently selected from 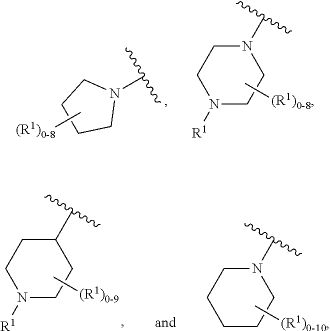
wherein R¹ is as described in claim 58.

84. The compound of any one of the preceding claims, wherein one of A and B is selected from 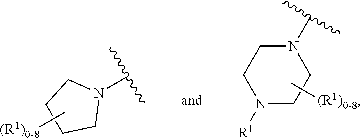
wherein R¹ is as described in claim 58.

85. The compound of any one of the preceding claims, wherein A is selected from 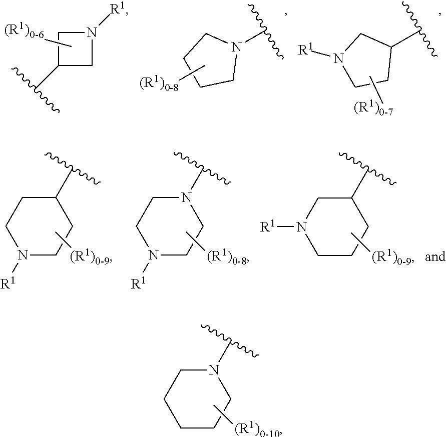
wherein R¹ is as described in claim 1.

86. The compound of any one of the preceding claims, wherein B is selected from 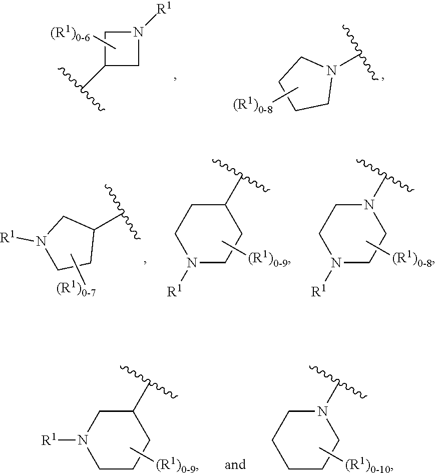
wherein R¹ is as described in claim 58.

87. The compound of any one of the preceding claims, wherein B is selected from 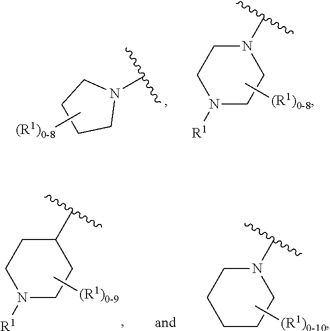
wherein R¹ is as described in claim 58.

88. The compound of any one of the preceding claims, wherein B is 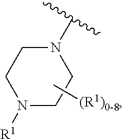
wherein R¹ is as described in claim 58.

89. The compound of any one of the preceding claims, wherein one of A and B is independently selected from

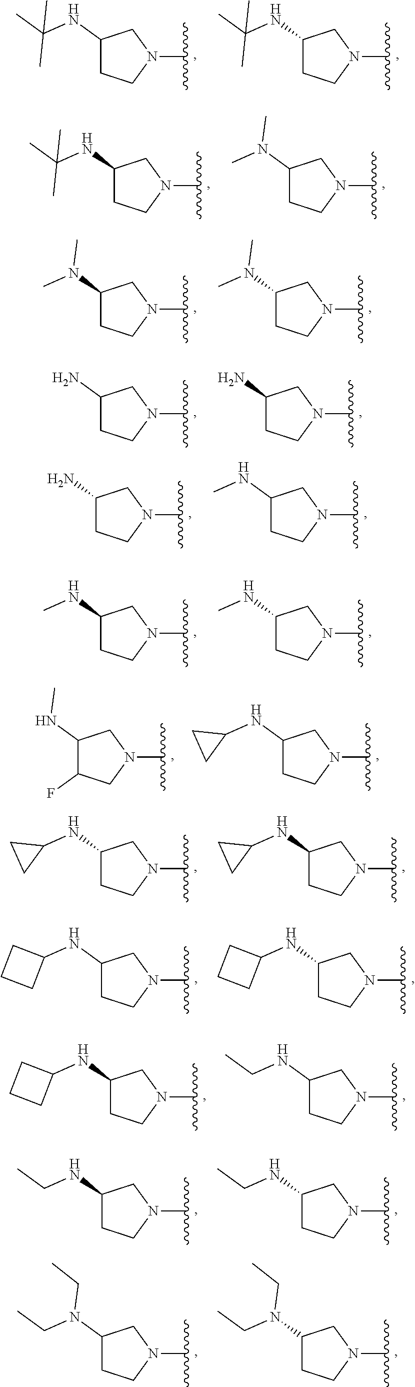

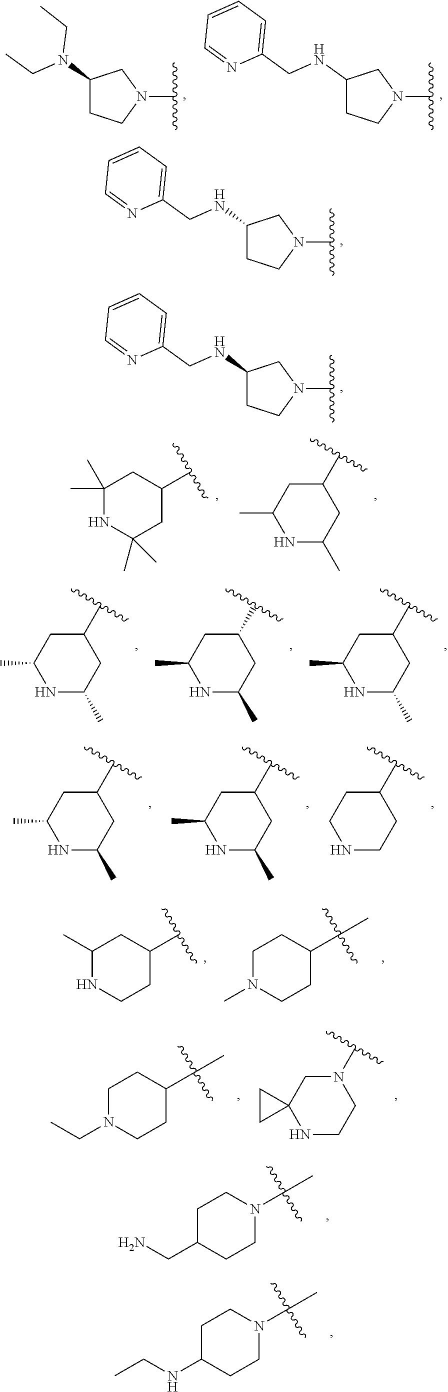

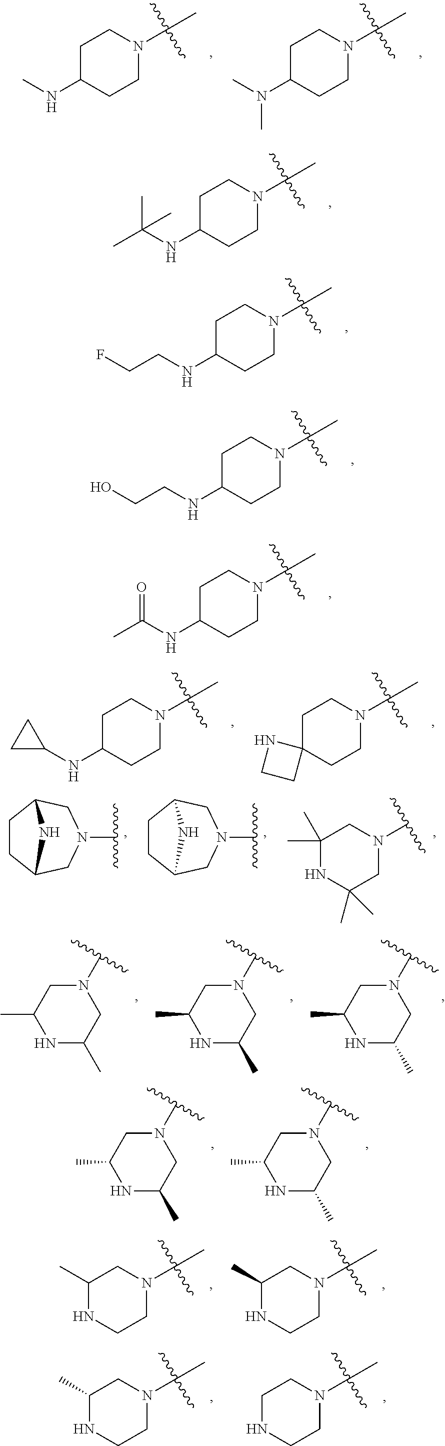

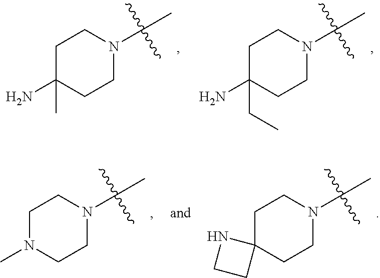

90. The compound of any one of the preceding claims, wherein one of A and B is independently selected from,

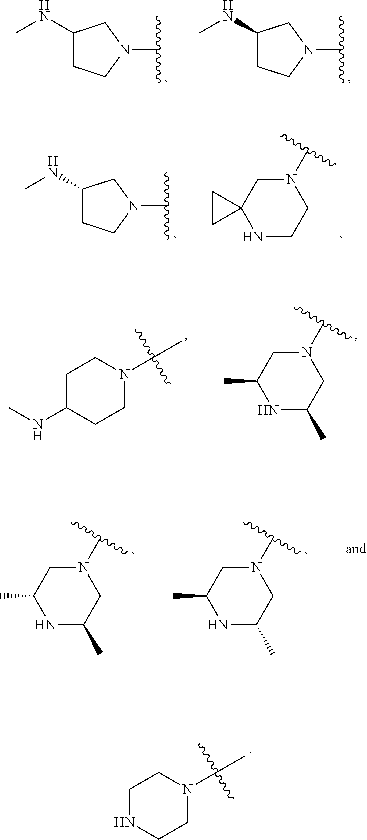

91. The compound of any one of the preceding claims, wherein one of A and B is selected from

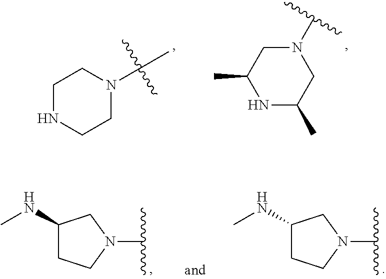

92. The compound of any one of the preceding claims, wherein A is selected from

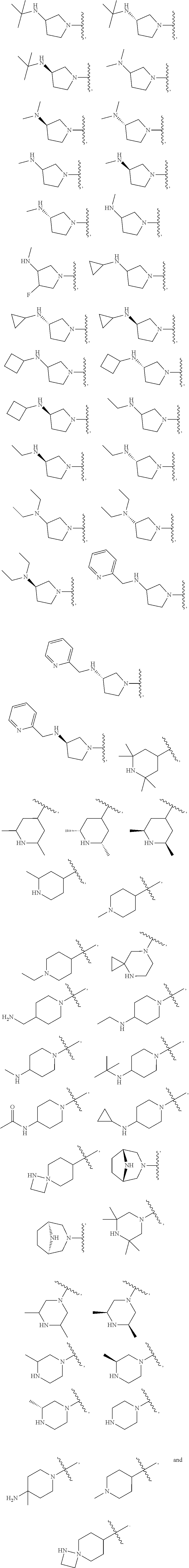

93. The compound of any one of the preceding claims, wherein B is selected from

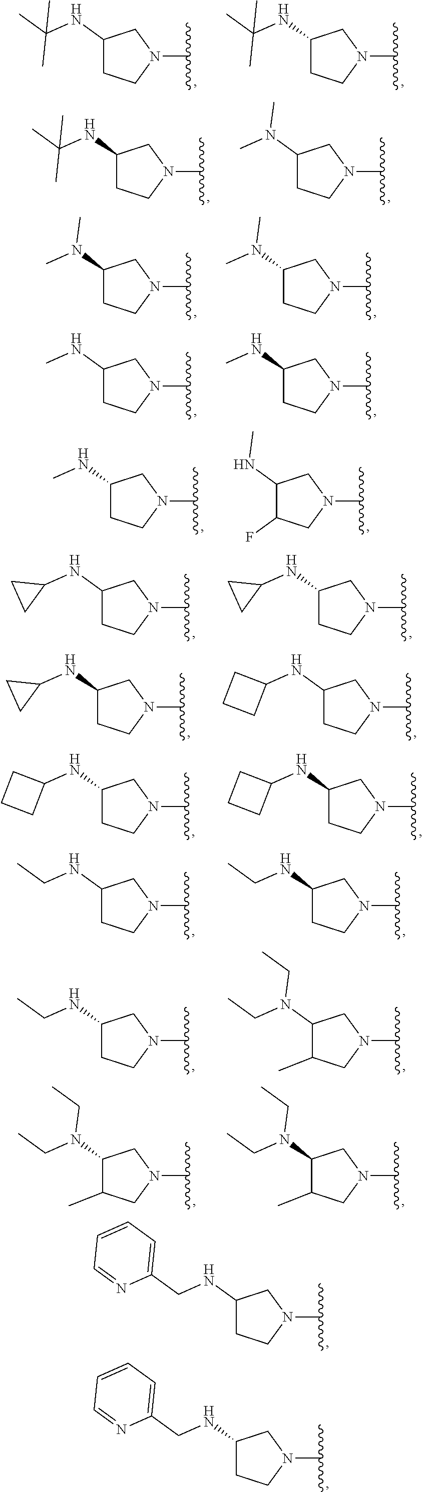

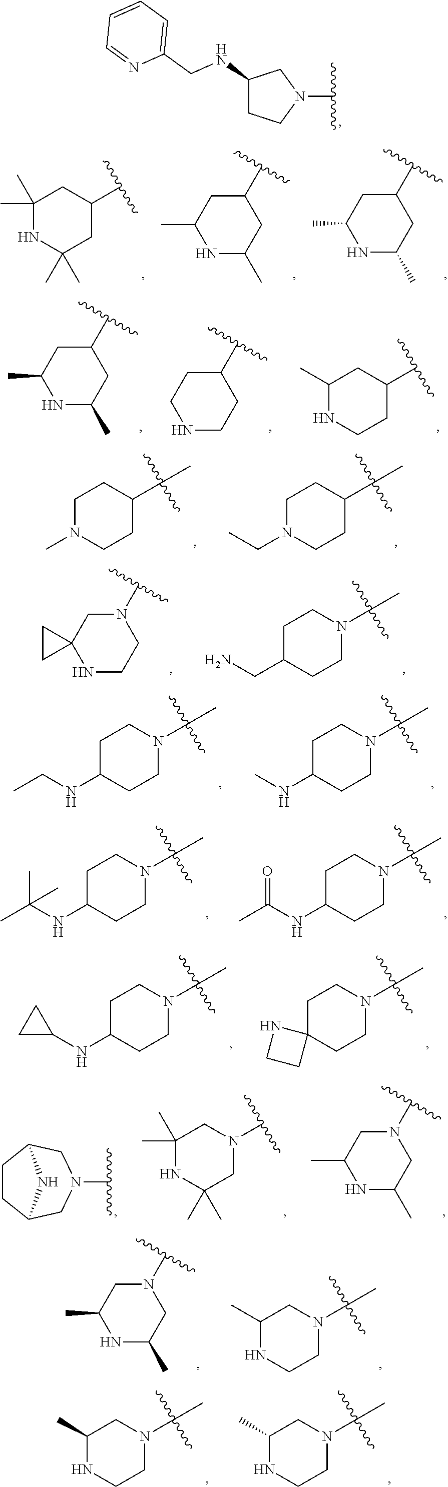

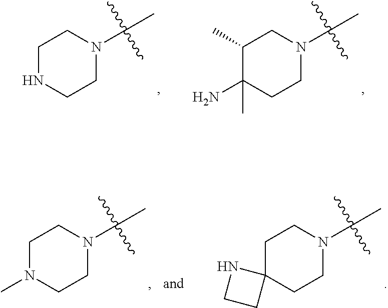

94. The compound of any one of the preceding claims, wherein B is selected from,

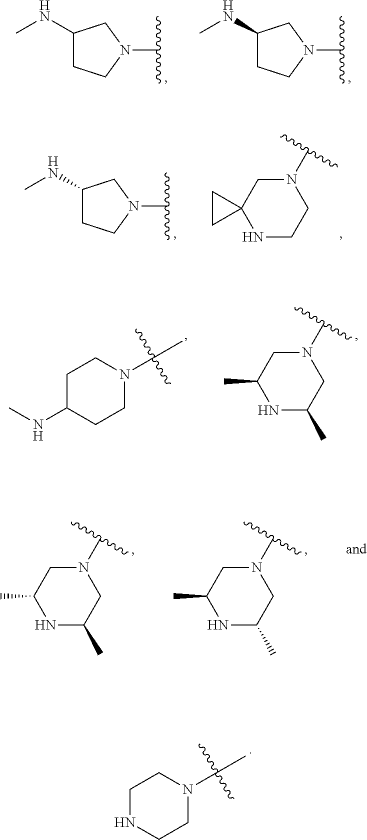

95. The compound of any one of the preceding claims, wherein B is selected from

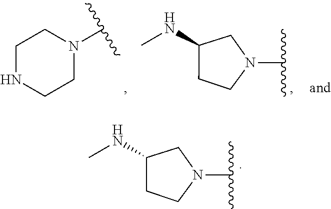

96. The compound of any one of the preceding claims, wherein each of L¹ and L² is independently absent, —N(R³)— (e.g., —N(CH₃)—), C₆-C₁₂-arylene, —N(R⁴)C(O)—, or —C(O)N(R⁴)—, wherein arylene is optionally substituted with one or more R¹.

97. The compound of any one of the preceding claims, wherein one of L¹ and L² is independently absent, —N(R⁴)C(O)—, or —C(O)N(R⁴)—.

98. The compound of any one of the preceding claims, wherein each of L¹ and L² is independently absent.

99. The compound of any one of the preceding claims, wherein L¹ is —C(O)N(R⁴)—.

100. The compound of any one of the preceding claims, wherein L² is absent.

101. The compound of any one of the preceding claims, wherein L¹ is —C(O)NH— and L² is absent.

102. The compound of any one of the preceding claims, wherein each of M and P is independently C(R²) (e.g., CH).

103. The compound of any one of the preceding claims, wherein M is C(R²) (e.g., CH) and P is N.

104. The compound of any one of the preceding claims, wherein M is N and P is C(R²) (e.g., CH).

105. The compound of any one of the preceding claims, wherein each of U and W is independently C.

106. The compound of any one of the preceding claims, wherein U is C and W is N.

107. The compound of any one of the preceding claims, wherein U is N and W is C.

108. The compound of any one of the preceding claims, wherein one of X, Y, and Z is C(R^{3a}) (e.g., CH), and the others of X, Y, and Z are each independently N, N(R^{3c}), or S.

109. The compound of any one of the preceding claims, wherein two of X, Y, and Z are independently N or N(R^{3c}).

110. The compound of any one of the preceding claims, wherein X and Y are each independently N or N(R^{3c}), and Z is C(R^{3a}) (e.g., CH).

111. The compound of any one of the preceding claims, wherein X is C(R^{3a}) (e.g., CH), and Y and Z are each independently N or N(R^{3c}).

112. The compound of any one of the preceding claims, wherein two of X, Y, and Z independently is C(R^{3a}) (e.g., CH).

113. The compound of any one of the preceding claims, wherein X is N and Z is S.

114. The compound of any one of the preceding claims, wherein X is N and Y is S.

115. The compound of any one of the preceding claims, wherein 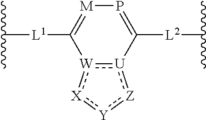
is selected from

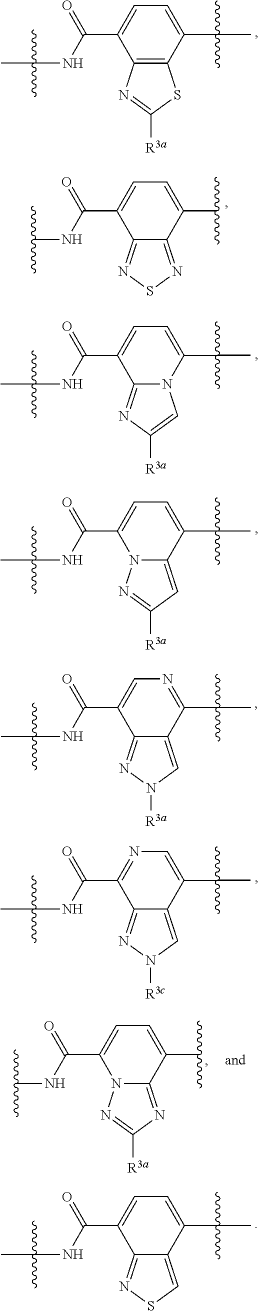

116. The compound of any one of the preceding claims, wherein the compound of Formula (II) is Formula (II-a): 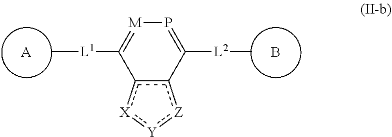
or a pharmaceutically acceptable salt, solvate, hydrate, tautomer, or stereoisomer thereof, wherein each of A, B, X, Y, Z, M, P, L¹, L², and subvariables thereof are defined as in claim 58.

117. The compound of any one of the preceding claims, wherein the compound of Formula (II) is Formula (II-c): 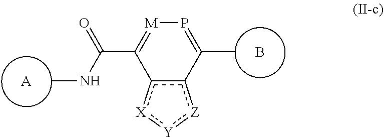
or a pharmaceutically acceptable salt, solvate, hydrate, tautomer, or stereoisomer thereof, wherein each of A, B, M, P, X, Y, Z, and subvariables thereof are defined as in claim 58.

118. The compound of any one of the preceding claims, wherein the compound of Formula (II) is Formula (II-d): 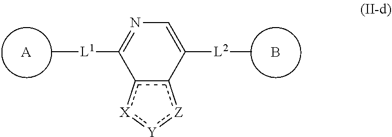
or a pharmaceutically acceptable salt, solvate, hydrate, tautomer, or stereoisomer thereof, wherein each of A, B, X, Y, Z, M, P, L¹, L², and subvariables thereof are defined as in claim 58.

119. The compound of any one of the preceding claims, wherein the compound of Formula (II) is Formula (II-e): 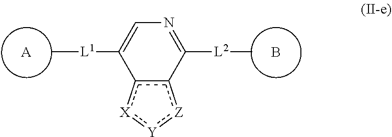
or a pharmaceutically acceptable salt, solvate, hydrate, tautomer, or stereoisomer thereof, wherein each of A, B, X, Y, Z, M, P, L¹, L², and subvariables thereof are defined as in claim 58.

120. The compound of any one of the preceding claims, wherein the compound of Formula (II) is Formula (II-f): 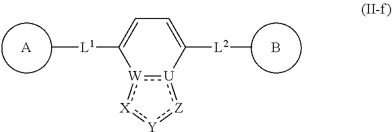
or a pharmaceutically acceptable salt, solvate, hydrate, tautomer, or stereoisomer thereof, wherein each of A, B, X, Y, Z, U, W, L¹, L², and subvariables thereof are defined as in claim 58.

121. The compound of any one of the preceding claims, wherein the compound of Formula (II) is Formula (II-g): 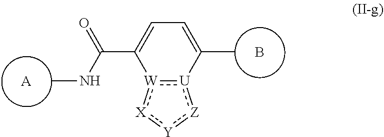
or a pharmaceutically acceptable salt, solvate, hydrate, tautomer, or stereoisomer thereof, wherein each of A, B, W, U, X, Y, Z, and subvariables thereof are defined as in claim 58.

122. The compound of any one of the preceding claims, wherein the compound of Formula (II) is Formula (II-h): 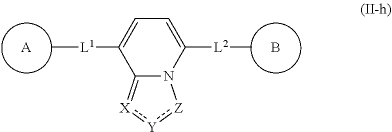
or a pharmaceutically acceptable salt, solvate, hydrate, tautomer, or stereoisomer thereof, wherein each of A, B, X, Y, Z, L¹, L², and subvariables thereof are defined as in claim 58.

123. The compound of any one of the preceding claims, wherein the compound of Formula (II) is Formula (II-i): 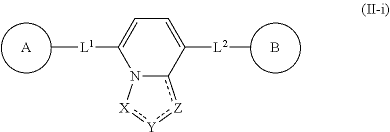
or a pharmaceutically acceptable salt, solvate, hydrate, tautomer, or stereoisomer thereof, wherein each of A, B, X, Y, Z, L¹, L², and subvariables thereof are defined as in claim 58.

124. The compound of any one of the preceding claims, wherein the compound of Formula (II) is Formula (II-j): 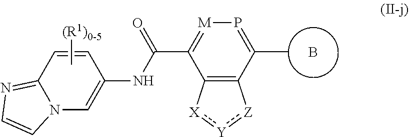
or a pharmaceutically acceptable salt, solvate, hydrate, tautomer, or stereoisomer thereof, wherein each of B, M, P, X, Y, Z, R¹, and subvariables thereof are defined as in claim 58.

125. The compound of any one of the preceding claims, wherein the compound of Formula (II) is Formula (II-k): 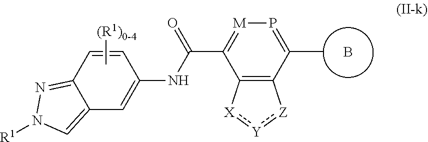
or a pharmaceutically acceptable salt, solvate, hydrate, tautomer, or stereoisomer thereof, wherein each of B, M, P, X, Y, Z, R¹, and subvariables thereof are defined as in claim 58.

126. The compound of any one of the preceding claims, wherein the compound of Formula (II) is Formula (II-l): 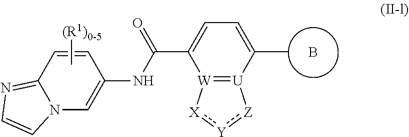
or a pharmaceutically acceptable salt, solvate, hydrate, tautomer, or stereoisomer thereof, wherein each of B, W, U, X, Y, Z, R¹, and subvariables thereof are defined as in claim 58.

127. The compound of any one of the preceding claims, wherein the compound of Formula (II) is Formula (II-m): 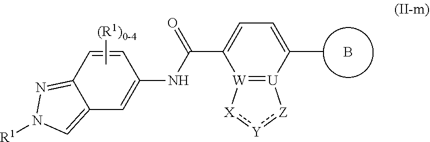
or a pharmaceutically acceptable salt, solvate, hydrate, tautomer, or stereoisomer thereof, wherein each of B, W, U, X, Y, Z, R¹, and subvariables thereof are defined as in claim 58.

128. The compound of any one of the preceding claims, wherein the compound is selected from any one of the compounds shown in Table 1 or a pharmaceutically acceptable salt, solvate, hydrate, tautomer, or stereoisomer thereof.

129. A pharmaceutical composition comprising a compound of any one of the preceding claims and a pharmaceutically acceptable excipient.

130. The compound of any one of claims 1-128, or the pharmaceutical composition of claim 129, wherein the compound alters a target nucleic acid (e.g., an RNA, e.g., a pre-mRNA).

131. The compound of any one of claims 1-128, or the pharmaceutical composition of claim 129, wherein the compound binds to a target nucleic acid (e.g., an RNA, e.g., a pre-mRNA).

132. The compound of any one of claims 1-128, or the pharmaceutical composition of claim 129, wherein the compound stabilizes a target nucleic acid (e.g., an RNA, e.g., a pre-mRNA).

133. The compound of any one of claims 1-128, or the pharmaceutical composition of claim 129, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

134. The compound of any one of claims 1-128, or the pharmaceutical composition of claim 129, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR %.

135. A method of forming a complex comprising a component of a spliceosome (e.g., a major spliceosome component or a minor spliceosome component), a nucleic acid (e.g., a DNA, RNA, e.g., a pre-mRNA), and a compound of Formula (I) or (II) or a composition thereof according to any one of claims 1-129:
comprising contacting the nucleic acid (e.g., a DNA, RNA, e.g., a pre-mRNA) with a compound of Formula (I) or (II).

136. The method of claim 135, wherein the component of a spliceosome is recruited to the nucleic acid in the presence of the compound of Formula (I) or (II).

137. A method of altering the conformation of a nucleic acid (e.g., a DNA, RNA, e.g., a pre-mRNA) comprising contacting the nucleic acid with a compound of Formula (I) or (II) according to any one of claims 1-128, or the pharmaceutical composition of claim 129.

138. The method of claim 137, wherein the altering comprises forming a bulge in the nucleic acid.

139. The method of claim 137, wherein the altering comprises stabilizing a bulge in the nucleic acid.

140. The method of claim 137, wherein the altering comprises reducing a bulge in the nucleic acid.

141. The method of any one of any one of claims 137-140, wherein the nucleic acid comprises a splice site.

142. A method for treating a disease or disorder in a subject comprising administering to the subject a compound of Formula (I) or (II) according to any one of claims 1-128 or the pharmaceutical composition of claim 129.

143. The method of claim 142, wherein the disease or disorder comprises a proliferative disease (e.g., cancer, a benign neoplasm, or angiogenesis).

144. The method of any one of claims 142-143, wherein the proliferative disease is cancer.

145. The method of any one of claims 142-143, wherein the proliferative disease comprises adenoid cystic carcinoma, colorectal cancer, leukemia, lung cancer, prostate cancer, breast cancer, or ovarian cancer

146. The method of claim 142, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

147. The method of claim 146, wherein the disease or disorder comprises a neurological disease or disorder.

148. The method of claim 146, wherein the disease or disorder comprises Huntington's disease.
